(19) United States
(12) Patent Application Publication
(10) Pub. No.: US 2026/0021181 A1
(43) Pub. Date: Jan. 22, 2026

(54) CHIMERIC COSTIMULATORY RECEPTORS, CHEMOKINE RECEPTORS, AND THE USE OF SAME IN CELLULAR IMMUNOTHERAPIES

(71) Applicant: Iovance Biotherapeutics, Inc., San Carlos, CA (US)

(72) Inventors: Rafael Cubas, San Carlos, CA (US); Frederick G. Vogt, Philadelphia, PA (US); Parameswaran Hari, San Carlos, CA (US)

(21) Appl. No.: 18/996,872

(22) PCT Filed: Jul. 20, 2023
(86) PCT No.: PCT/US2023/070616

§ 371 Date: Jan. 17, 2025

Related U.S. Application Data
(60) Provisional application No. 63/394,241, filed on Aug. 1, 2022.
(60) Provisional application No. 63/380,228, filed on Oct. 19, 2022.

(51) Int. Cl. A61K 40/11; A61K 31/675; A61K 31/7076; A61K 35/17; A61K 38/20; A61K 40/42; A61P 35/04; C07K 14/715; C07K 16/00; C12N 5/00; C12N 5/0783; C12N 5/10

(52) U.S. Cl. CPC A61K 40/11; A61K 31/675; A61K 31/7076; A61K 35/17; A61K 38/2013; A61K 40/42; A61P 35/04; C07K 14/7158; C07K 16/00; C12N 5/0638; C12N 5/10; C12N 5/562; C07K 2319/03; C12N 2501/2302; C12N 2501/2304; C12N 2501/2307; C12N 2501/2315; C12N 2501/2321; C12N 2502/11; C12N 2510/00

(57) ABSTRACT

The present invention provides compositions comprising chimeric receptors, including chimeric costimulatory receptors (CCRs), and/or chemokine receptors, methods for preparing CCRs and/or chemokine receptors, and therapeutic populations of tumor infiltrating lymphocytes, marrow infiltrating lymphocytes, and peripheral blood lymphocytes expressing CCRs and/or chemokine receptors with increased therapeutic performance and other advantages for the treatment of cancers, including solid tumor cancers.

### BACKGROUND OF THE INVENTION

This application claims priority to and the benefit of U.S. Patent Application Nos. 63/394,241, filed Aug. 1, 2022, and 63/380,228, filed Oct. 19, 2022, which are incorporated herein by reference in their entirety.

### BACKGROUND OF THE INVENTION

Treatment of solid tumor cancers remains challenging, particularly for patients that do not respond to commonly-used initial lines of therapy, including chemotherapy, targeted therapies, and checkpoint inhibitors such as nivolumab, pembrolizumab, ipilimumab, atezolizumab, avelumab, durvalumab, and therapy using combinations of these immunotherapies (such as nivolumab and ipilimumab) or combinations of these immunotherapies with chemotherapy. Treatment of bulky, refractory cancers using adoptive transfer of tumor infiltrating lymphocytes (TILs) represents a powerful approach to therapy for solid tumor cancer patients with poor prognoses, including those that fail initial lines of therapy. Gattinoni, et al., Nat. Rev. Immunol. 2006, 6, 383-393. A large number of active TILs are required for successful immunotherapy, and a robust and reliable process is needed for commercialization. This has been a challenge to achieve because of technical, logistical, and regulatory issues with cell expansion. IL-2-based TIL expansion followed by a rapid expansion process (REP) has become a preferred method for TIL expansion because of its speed and efficiency. Dudley, et al., Science 2002, 298, 850-54; Dudley, et al., J. Clin. Oncol. 2005, 23, 2346-57; Dudley, et al., J. Clin. Oncol. 2008, 26, 5233-39; Riddell, et al., Science 1992, 257, 238-41; Dudley, et al., J. Immunother. 2003, 26, 332-42. REP can result in a 1,000-fold expansion of TILs over a 14-day period, although it requires a large excess (e.g., 200-fold) of irradiated allogeneic peripheral blood mononuclear cells (PBMCs, also known as mononuclear cells (MNCs)), often from multiple donors, as feeder cells, as well as anti-CD3 antibody (OKT3) and high doses of IL-2. Dudley, et al., J. Immunother. 2003, 26, 332-42. TILs that have undergone an REP procedure have produced successful adoptive cell therapy following host immunosuppression in patients with melanoma, head and neck cancer, non-small cell lung cancer, and cervical cancer in both a front-line and later line of therapy setting. Jimeno, et al., Poster 353, SITC Annual Meeting, Nov. 9-14, 2020; Sarnaik, et al., Oral Presentation at ASCO Annual Meeting, May 29-30, 2020; Jazaeri, et al., Poster 182, ASCO Annual Meeting, May 31-Jun. 4, 2019.

Chimeric costimulatory receptors (CCRs) are genetically engineered chimeric receptors designed to provide costimulatory signals to effector cells, such as T-cells, to boost activation. Sadelain, et al., Cancer Discovery, 2013, 3, 388-398; Liao, et al., Riomarker Res. 2020, 8, 57. CCRs are most commonly associated with T-cell therapies based on chimeric antigen receptor (CAR) modified T-cell (CAR-T) products, where they can be used in combination with CARs to enhance activation. However, CCRs have not yet been extensively explored with other emerging cell therapies, including polyclonal TIL therapies in solid tumor cancers, for example using tumor-associated antigens or other antigens for activation. Although current TIL therapies have demonstrated safety and efficacy, a significant unmet need exists for TIL therapies with improved efficacy, duration of response, and safety, among other factors, particularly for patients with locally advanced or metastatic solid tumor cancers.

### BRIEF SUMMARY OF THE INVENTION

The present invention provides methods for making and using TILs with enhanced properties using CCRs, and compositions thereof, for use in treatment of solid tumor patients.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the extracellular domain comprises an scFv binding domain.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the extracellular domain comprises an scFv binding domain, and wherein the scFv binding domain binds to a protein selected from the group consisting of CD19, CD20, CD22, CD24, CD33, CD38, CD39, CD73, CD123, CD138, CD228, LRRC15, CEA, FRα, EPCAM, DLL3, PD-L1, PSMA, gp100, MUC1, MCSP, EGFR, GD2, TROP-2, GPC3, MICA, MICB, VISTA, ULBP, HER2, MCM5, FAP, 5T4, LFA-1, B7-H3, IL-13Rα2, FAS, TGFβRII, and MUC16.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the extracellular domain is a PD-1 domain.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the intracellular domain is selected from the group consisting of CD28, CD134 (OX40), CD278 (ICOS), CD137 (4-1BB), CD27, IL-2Rβ, IL-2Rγ, IL-18R1, IL-7Rα, IL-12R1, IL-12R2, IL-15Rα, IL-21R, and combinations thereof.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the intracellular domain is selected from the group consisting of CD28, CD134 (OX40), CD278 (ICOS), CD137 (4-1BB), CD27, IL-2Rβ, IL-2Rγ, IL-18R1, IL-7Rα, IL-12R1, IL-12R2, IL-15Rα, IL-21R, and combinations thereof, and wherein the transmembrane domain is selected from the group consisting of the transmembrane region of CD3α, CD3β, CDζ, CD3ε, CD4, CD5, CD8a, CD9, CD16, CD22, CD27, CD28, CD33, CD37, CD45, CD64, CD80, CD86, CD134, CD137, CD154, IgG1, IgG4, IgD, IL-2Rα, IL-2Rβ, and IL-2Rγ.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the intracellular domain is selected from the group consisting of CD28, CD134 (OX40), CD278 (ICOS), CD137 (4-1BB), CD27, IL-2Rβ, IL-2Rγ, IL-18R1, IL-7Rα, IL-12R1, IL-12R2, IL-15Rα, IL-21R, and combinations thereof, wherein the transmembrane domain is selected from the group consisting of the transmembrane region of CD3α, CD3β, CDζ, CD3ε, CD4, CD5, CD8α, CD9, CD16, CD22, CD27, CD28, CD33, CD37, CD45, CD64, CD80, CD86, CD134, CD137, CD154, IgG1, IgG4, IgD, IL-2Rα, IL-2Rβ, and IL-2Rγ, and wherein step (d) further comprises genetically modifying TILs using a lentivirus to express the CCR.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the intracellular domain is selected from the group consisting of CD28, CD134 (OX40), CD278 (ICOS), CD137 (4-1BB), CD27, IL-2Rβ, IL-2Rγ, IL-18R1, IL-7Rα, IL-12R1, IL-12R2, IL-15Rα, IL-21R, and combinations thereof, wherein the transmembrane domain is selected from the group consisting of the transmembrane region of CD3α, CD3β, CDζ, CD3ε, CD4, CD5, CD8α, CD9, CD16, CD22, CD27, CD28, CD33, CD37, CD45, CD64, CD80, CD86, CD134, CD137, CD154, IgG1, IgG4, IgD, IL-2Rα, IL-2Rβ, and IL-2Rγ, wherein step (d) further comprises genetically modifying TILs using a lentivirus to express the CCR, and wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), SOCS1, ANKRD11, BCOR, and combinations thereof.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient,
   wherein the intracellular domain is selected from the group consisting of CD28, CD134 (OX40), CD278 (ICOS), CD137 (4-1BB), CD27, IL-2Rβ, IL-2Rγ, IL-18R1, IL-7Rα, IL-12R1, IL-12R2, IL-15Rα, IL-21R, and combinations thereof, wherein the transmembrane domain is selected from the group consisting of the transmembrane region of CD3α, CD3β, CDζ, CD3ε, CD4, CD5, CD8α, CD9, CD16, CD22, CD27, CD28, CD33, CD37, CD45, CD64, CD80, CD86, CD134, CD137, CD154, IgG1, IgG4, IgD, IL-2Rα, IL-2Rβ, and IL-2Rγ, wherein step (d) further comprises genetically modifying TILs using a lentivirus to express the CCR, wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), SOCS1, ANKRD11, BCOR, and combinations thereof, and wherein the cancer is a solid tumor cancer treated by administration of TILs.

In any of the foregoing embodiments, the cancer may be selected from the group consisting of sarcoma, pancreatic cancer, liver cancer, glioblastoma, gastrointestinal cancer, melanoma, ovarian cancer, endometrial cancer, thyroid cancer, colorectal cancer, cervical cancer, lung cancer, non-small-cell lung cancer, small-cell lung cancer, mesothelioma, bladder cancer, breast cancer, cancer caused by human papilloma virus, head and neck cancer, renal cancer, and renal cell carcinoma, and wherein the patient is a human.

In any of the foregoing embodiments, the cancer is further treated in combination with TILs using a PD-1 inhibitor or PD-L1 inhibitor, wherein the PD-1 or PD-L1 inhibitor is selected from the group consisting of nivolumab, pembrolizumab, cemiplimab, tislelizumab, sintilimab, toripalimab, dostarlimab, durvalumab, avelumab, atezolizumab, retifanlimab, and fragments, variants, and biosimilars thereof.

In any of the foregoing embodiments, the cancer is non-small-cell lung cancer, wherein the patient has at least one of
1) a predetermined tumor proportion score (TPS) of PD-L1 of <1%,
2) a TPS score of PD-L1 of 1%-49%, or
3) a predetermined absence of one or more driver mutations.

In any of the foregoing embodiments, the cancer is non-small-cell lung cancer, wherein the patient has a TPS of PD-L1 of <1%.

In any of the foregoing embodiments, the patient has a cancer that is not indicated for treatment by an EGFR inhibitor, a BRAF inhibitor, an ALK inhibitor, a c-Ros inhibitor, a RET inhibitor, an ERBB2 inhibitor, BRCA inhibitor, a MAP2K1 inhibitor, PIK3CA inhibitor, CDKN2A inhibitor, a PTEN inhibitor, an UMD inhibitor, an NRAS inhibitor, a KRAS inhibitor, an NF1 inhibitor, MET inhibitor a TP53 inhibitor, a CREBBP inhibitor, a KMT2C inhibitor, a KMT2D mutation, an ARID1A mutation, a RB1 inhibitor, an ATM inhibitor, a SETD2 inhibitor, a FLT3 inhibitor, a PTPN11 inhibitor, a FGFR1 inhibitor, an EP300 inhibitor, a MYC inhibitor, an EZH2 inhibitor, a JAK2 inhibitor, a FBXW7 inhibitor, a CCND3 inhibitor, and a GNA11 inhibitor.

In any of the foregoing embodiments, the patient has an absence of one or more driver mutations, wherein the one or more driver mutations is selected from the group consisting of an EGFR mutation, an EGFR insertion, EGFR exon20, a KRAS mutation, a BRAF-mutation, a BRAF V600 mutation, an ALK-mutation, a c-ROS-mutation (ROS1-mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation.

In any of the foregoing embodiments, the cancer is refractory or resistant to treatment with a chemotherapeutic agent or chemotherapeutic regimen.

In any of the foregoing embodiments, the cancer is refractory or resistant to treatment with a VEGF-A inhibitor, wherein the VEGF-A inhibitor is selected from the group consisting of bevacizumab, ranibizumab, icrucumab, and fragments, variants, and biosimilars thereof.

In any of the foregoing embodiments, the cancer is refractory or resistant to treatment with a PD-1 inhibitor or PD-L1 inhibitor, wherein the PD-1 or PD-L1 inhibitor is selected from the group consisting of nivolumab, pembrolizumab, cemiplimab, tislelizumab, sintilimab, toripalimab, dostarlimab, durvalumab, avelumab, atezolizumab, retifanlimab, and fragments, variants, and biosimilars thereof.

In any of the foregoing embodiments, the cancer is refractory or resistant to treatment with a CTLA-4 inhibitor, wherein the CTLA-4 inhibitor is selected from the group consisting of ipilimumab, tremelimumab, zalifrelimab, and fragments, variants, and biosimilars thereof.

In any of the foregoing embodiments, the IL-2 is initially present at an initial concentration of between 1000 IU/mL and 6000 IU/mL in the first cell culture medium and in the second cell culture medium.

In any of the foregoing embodiments, the OKT-3 antibody is initially present at an initial concentration of about 30 ng/mL in the second cell culture medium.

In any of the foregoing embodiments, the first cell culture medium further comprises a cytokine selected from the group consisting of IL-4, IL-7, IL-15, IL-21, and combinations thereof.

In any of the foregoing embodiments, the second cell culture medium further comprises a cytokine selected from the group consisting of IL-4, IL-7, IL-15, IL-21, and combinations thereof.

In any of the foregoing embodiments, the method may further comprise the step of treating the patient with a non-myeloablative lymphodepletion regimen prior to administering the third population of TILs to the patient.

In any of the foregoing embodiments, the method may further comprise the step of treating the patient with a non-myeloablative lymphodepletion regimen prior to administering the third population of TILs to the patient, wherein the non-myeloablative lymphodepletion regimen comprises the steps of administration of cyclophosphamide at a dose of 60 mg/m²/day for two days followed by administration of fludarabine at a dose of 25 mg/m²/day for five days.

In any of the foregoing embodiments, the method may further comprise the step of treating the patient with a non-myeloablative lymphodepletion regimen prior to administering the third population of TILs to the patient, wherein the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In any of the foregoing embodiments, the method may further comprise the step of treating the patient with an IL-2 regimen starting on the day after administration of the third population of TILs to the patient.

In any of the foregoing embodiments, the method may further comprise the step of treating the patient with an IL-2 regimen starting on the same day as administration of the third population of TILs to the patient.

In any of the foregoing embodiments, the method may further comprise the step of treating the patient with an IL-2 regimen, wherein the IL-2 regimen is a high-dose IL-2 regimen comprising 600,000 or 720,000 IU/kg of aldesleukin, or a fragment, variant, or biosimilar thereof, administered as a 15-minute bolus intravenous infusion every eight hours until tolerance.

In any of the foregoing embodiments, the method may further comprise the step of treating the patient with an IL-2 regimen, wherein the IL-2 regimen comprises administration of bempegaldesleukin, or a fragment, variant, or biosimilar thereof.

In any of the foregoing embodiments, the method may further comprise the step of treating the patient with an IL-2 regimen, wherein the IL-2 regimen comprises administration of THOR-707, or a fragment, variant, or biosimilar thereof.

In any of the foregoing embodiments, the method may further comprise the step of treating the patient with an IL-2 regimen, wherein the IL-2 regimen comprises administration of nemvaleukin alfa, or a fragment, variant, or biosimilar thereof.

In any of the foregoing embodiments, the method may further comprise the step of treating the patient with an IL-2 regimen, wherein the IL-2 regimen comprises administration of an antibody comprising a heavy chain selected from the group consisting of SEQ ID NO: 29 and SEQ ID NO:38 and a light chain selected from the group consisting of SEQ ID NO:37 and SEQ ID NO:39, or a fragment, variant, or biosimilar thereof.

In any of the foregoing embodiments, a therapeutically effective population of TILs is administered and comprises from about 2×10⁹ to about 15×10¹⁰ TILs.

In any of the foregoing embodiments, the first expansion is performed over a period of 11 days or less.

In any of the foregoing embodiments, the second expansion is performed over a period of 11 days or less.

In some embodiments, the present invention includes a composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain.

In some embodiments, the present invention includes a composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the extracellular domain comprises an scFv binding domain.

In some embodiments, the present invention includes a composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the extracellular domain comprises an scFv binding domain, wherein the scFv binding domain is selected from the group consisting of an anti-DLL3 domain, an anti-CD19 domain, an anti-CD20 domain, an anti-CD22 domain, an anti-CD24 domain, an anti-CD33 domain, an anti-CD38 domain, an anti-CD39 domain, an anti-CD73 domain, an anti-CD123 domain, an anti-CD138 domain, an anti-CD228 domain, an anti-LRRC15 domain, an anti-CEA domain, an anti-FRα domain, an anti-EPCAM domain, an anti-PD-L1 domain, an anti-PSMA domain, an anti-gp100 domain, an anti-MUC1 domain, an anti-MCSP domain, an anti-EGFR domain, an anti-GD2 domain, an anti-TROP-2 domain, an anti-GPC3 domain, an anti-MICA domain, an anti-MICB domain, an anti-VISTA domain, an anti-ULBP domain, an anti-HER2 domain, an anti-MCM5 domain, an anti-FAP domain, an anti-5T4 domain, an anti-LFA-1 domain, an anti-B7-H3 domain, and an anti-MUC16 domain.

In some embodiments, the present invention includes a composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the extracellular domain is a PD-1 domain.

In some embodiments, the present invention includes a composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the intracellular domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof.

In some embodiments, the present invention includes a composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the intracellular domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof, and wherein the transmembrane domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, a IgG1 domain, a IgG4 domain, a IgD domain, a IL-2Rα domain, a IL-2Rβ domain, and a IL-2Rγ domain.

In some embodiments, the present invention includes a composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. Optionally, a hinge domain,
iii. Optionally, a transmembrane domain, and
iv. At least one intracellular domain,
   wherein the intracellular domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof, wherein the transmembrane domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, a IgG1 domain, a IgG4 domain, a IgD domain, a IL-2Rα domain, a IL-2Rβ domain, and a IL-2Rγ domain, and wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), and combinations thereof.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the extracellular protein domain comprises an scFv binding domain.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the extracellular protein domain comprises an scFv binding domain, wherein the scFv binding domain is selected from the group consisting of an anti-DLL3 domain, an anti-CD19 domain, an anti-CD20 domain, an anti-CD22 domain, an anti-CD24 domain, an anti-CD33 domain, an anti-CD38 domain, an anti-CD39 domain, an anti-CD73 domain, an anti-CD123 domain, an anti-CD138 domain, an anti-CD228 domain, an anti-LRRC15 domain, an anti-CEA domain, an anti-FRα domain, an anti-EPCAM domain, an anti-PD-L1 domain, an anti-PSMA domain, an anti-gp100 domain, an anti-MUC1 domain, an anti-MCSP domain, an anti-EGFR domain, an anti-GD2 domain, an anti-TROP-2 domain, an anti-GPC3 domain, an anti-MICA domain, an anti-MICB domain, an anti-VISTA domain, an anti-ULBP domain, an anti-HER2 domain, an anti-MCM5 domain, an anti-FAP domain, an anti-5T4 domain, an anti-LFA-1 domain, an anti-B7-H3 domain, and an anti-MUC16 domain.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the extracellular protein domain is a PD-1 domain.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the intracellular protein domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the intracellular protein domain is selected from the group consisting of a CD28 domain, a CD134 (OX40) domain, a CD278 (ICOS) domain, a CD137 (4-11B) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof, and wherein the transmembrane protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the intracellular protein domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof, wherein the transmembrane protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2R0 domain, and an IL-2Rγ domain, and wherein the hinge protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the intracellular protein domain is selected from the group consisting of a CD28 domain, a CD134 (OX40) domain, a CD278 (ICOS) domain, a CD137 (4-11B) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof, wherein the transmembrane protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, wherein the hinge protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, wherein the composition further comprises a tumor infiltrating lymphocyte.

In some embodiments, the present invention includes a composition comprising a chimeric costimulatory receptor (CCR), wherein the CCR comprises.
i. An extracellular protein domain,
ii. A hinge protein domain,
iii. A transmembrane protein domain, and
iv. At least one intracellular protein domain,
   wherein the intracellular protein domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof, wherein the transmembrane protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, wherein the hinge protein domain is selected from the group consisting of a CD3α domain, a CD3β domain, a CDC domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, wherein the composition further comprises a marrow infiltrating lymphocyte.

In some embodiments, the present invention includes a composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain,
   wherein the intracellular protein domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof, wherein the transmembrane protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, wherein the hinge protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, wherein the composition further comprises a peripheral blood lymphocyte.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chemokine receptor.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chemokine receptor, wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the chemokine receptor;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chemokine receptor, wherein the cancer is treated by administering a population of TILs, and wherein the method comprises, wherein the chemokine receptor is a protein selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chemokine receptor, wherein the cancer is treated by administering a population of TILs, wherein the method comprises, wherein the chemokine receptor is a protein selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof, and wherein step (d) further comprises genetically modifying TILs using a lentivirus or retrovirus to express the chemokine receptor.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chemokine receptor, wherein the cancer is treated by administering a population of TILs, wherein the method comprises, wherein the chemokine receptor is a protein selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof, wherein step (d) further comprises genetically modifying TILs using a lentivirus or retrovirus to express the chemokine receptor, and wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), SOCS1, ANKRD11, BCOR, and combinations thereof.

In some embodiments, the present invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chemokine receptor, wherein the cancer is a solid tumor cancer treated by administration of TILs.

In any of the foregoing embodiments, the cancer may be selected from the group consisting of sarcoma, pancreatic cancer, liver cancer, glioblastoma, gastrointestinal cancer, melanoma, ovarian cancer, endometrial cancer, thyroid cancer, colorectal cancer, cervical cancer, lung cancer, non-small-cell lung cancer, small-cell lung cancer, mesothelioma, bladder cancer, breast cancer, cancer caused by human papilloma virus, head and neck cancer, renal cancer, and renal cell carcinoma, and wherein the patient is a human.

In any of the foregoing embodiments, the cancer is further treated in combination with TILs using a PD-1 inhibitor or PD-L1 inhibitor, wherein the PD-1 or PD-L1 inhibitor is selected from the group consisting of nivolumab, pembrolizumab, cemiplimab, tislelizumab, sintilimab, toripalimab, dostarlimab, durvalumab, avelumab, atezolizumab, retifanlimab, and fragments, variants, and biosimilars thereof.

In any of the foregoing embodiments, the cancer is non-small-cell lung cancer, wherein the patient has at least one of
1) a predetermined tumor proportion score (TPS) of PD-L1 of <1%,
2) a TPS score of PD-L1 of 1%-49%, or
3) a predetermined absence of one or more driver mutations.

In any of the foregoing embodiments, the cancer is non-small-cell lung cancer, wherein the patient has a TPS of PD-L1 of <1%.

In any of the foregoing embodiments, the patient has a cancer that is not indicated for treatment by an EGFR inhibitor, a BRAF inhibitor, an ALK inhibitor, a c-Ros inhibitor, a RET inhibitor, an ERBB2 inhibitor, BRCA inhibitor, a MAP2K1 inhibitor, PIK3CA inhibitor, CDKN2A inhibitor, a PTEN inhibitor, an UMD inhibitor, an NRAS inhibitor, a KRAS inhibitor, an NF1 inhibitor, MET inhibitor a TP53 inhibitor, a CREBBP inhibitor, a KMT2C inhibitor, a KMT2D mutation, an ARID1A mutation, a RB1 inhibitor, an ATM inhibitor, a SETD2 inhibitor, a FLT3 inhibitor, a PTPN11 inhibitor, a FGFR1 inhibitor, an EP300 inhibitor, a MYC inhibitor, an EZH2 inhibitor, a JAK2 inhibitor, a FBXW7 inhibitor, a CCND3 inhibitor, and a GNA11 inhibitor.

In any of the foregoing embodiments, the patient has an absence of one or more driver mutations, wherein the one or more driver mutations is selected from the group consisting of an EGFR mutation, an EGFR insertion, EGFR exon20, a KRAS mutation, a BRAF-mutation, a BRAF V600 mutation, an ALK-mutation, a c-ROS-mutation (ROS I-mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation.

In any of the foregoing embodiments, the cancer is refractory or resistant to treatment with a chemotherapeutic agent or chemotherapeutic regimen.

In any of the foregoing embodiments, the cancer is refractory or resistant to treatment with a VEGF-A inhibitor, wherein the VEGF-A inhibitor is selected from the group consisting of bevacizumab, ranibizumab, icrucumab, and fragments, variants, and biosimilars thereof.

In any of the foregoing embodiments, the cancer is refractory or resistant to treatment with a PD-1 inhibitor or PD-L1 inhibitor, wherein the PD-1 or PD-L1 inhibitor is selected from the group consisting of nivolumab, pembrolizumab, cemiplimab, tislelizumab, sintilimab, toripalimab, dostarlimab, durvalumab, avelumab, atezolizumab, retifanlimab, and fragments, variants, and biosimilars thereof.

In any of the foregoing embodiments, the cancer is refractory or resistant to treatment with a CTLA-4 inhibitor, wherein the CTLA-4 inhibitor is selected from the group consisting of ipilimumab, tremelimumab, zalifrelimab, and fragments, variants, and biosimilars thereof.

In any of the foregoing embodiments, the IL-2 is initially present at an initial concentration of between 1000 IU/mL and 6000 IU/mL in the first cell culture medium and in the second cell culture medium.

In any of the foregoing embodiments, the OKT-3 antibody is initially present at an initial concentration of about 30 ng/mL in the second cell culture medium.

In any of the foregoing embodiments, the first cell culture medium further comprises a cytokine selected from the group consisting of IL-4, IL-7, IL-15, IL-21, and combinations thereof.

In any of the foregoing embodiments, the second cell culture medium further comprises a cytokine selected from the group consisting of IL-4, IL-7, IL-15, IL-21, and combinations thereof.

In any of the foregoing embodiments, the method may further comprise the step of treating the patient with a non-myeloablative lymphodepletion regimen prior to administering the third population of TILs to the patient.

In any of the foregoing embodiments, the method may further comprise the step of treating the patient with a non-myeloablative lymphodepletion regimen prior to administering the third population of TILs to the patient, wherein the non-myeloablative lymphodepletion regimen comprises the steps of administration of cyclophosphamide at a dose of 60 mg/m²/day for two days followed by administration of fludarabine at a dose of 25 mg/m²/day for five days.

In any of the foregoing embodiments, the method may further comprise the step of treating the patient with a non-myeloablative lymphodepletion regimen prior to administering the third population of TILs to the patient, wherein the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In any of the foregoing embodiments, the method may further comprise the step of treating the patient with an IL-2 regimen starting on the day after administration of the third population of TILs to the patient.

In any of the foregoing embodiments, the method may further comprise the step of treating the patient with an IL-2 regimen starting on the same day as administration of the third population of TILs to the patient.

In any of the foregoing embodiments, the method may further comprise the step of treating the patient with an IL-2 regimen, wherein the IL-2 regimen is a high-dose IL-2 regimen comprising 600,000 or 720,000 IU/kg of aldesleukin, or a fragment, variant, or biosimilar thereof, administered as a 15-minute bolus intravenous infusion every eight hours until tolerance.

In any of the foregoing embodiments, the method may further comprise the step of treating the patient with an IL-2 regimen, wherein the IL-2 regimen comprises administration of bempegaldesleukin, or a fragment, variant, or biosimilar thereof.

In any of the foregoing embodiments, the method may further comprise the step of treating the patient with an IL-2 regimen, wherein the IL-2 regimen comprises administration of THOR-707, or a fragment, variant, or biosimilar thereof.

In any of the foregoing embodiments, the method may further comprise the step of treating the patient with an IL-2 regimen, wherein the IL-2 regimen comprises administration of nemvaleukin alfa, or a fragment, variant, or biosimilar thereof.

In any of the foregoing embodiments, the method may further comprise the step of treating the patient with an IL-2 regimen, wherein the IL-2 regimen comprises administration of an antibody comprising a heavy chain selected from the group consisting of SEQ ID NO:29 and SEQ ID NO:38 and a light chain selected from the group consisting of SEQ ID NO:37 and SEQ ID NO:39, or a fragment, variant, or biosimilar thereof.

In any of the foregoing embodiments, a therapeutically effective population of TILs is administered and comprises from about 2×10⁹ to about 15×10¹⁰ TILs.

In any of the foregoing embodiments, the first expansion is performed over a period of 11 days or less.

In any of the foregoing embodiments, the second expansion is performed over a period of 11 days or less.

In some embodiments, the present invention provides a composition comprising a tumor infiltrating lymphocyte (TIL), marrow infiltrating lymphocyte (MIL), or peripheral blood lymphocyte (PBL) genetically modified to express a chemokine receptor.

In some embodiments, the present invention composition of Claim 94, wherein the chemokine receptor is a protein selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof.

In some embodiments, the composition of any one of Claims 94 to 95, wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), and combinations thereof.

In some embodiments, the present invention provides a composition comprising a chemokine receptor, wherein the composition further comprises a tumor infiltrating lymphocyte, a marrow infiltrating lymphocyte, or a peripheral blood lymphocyte.

In some embodiments, the present invention provides a method or composition according to any of the disclosures provided herein for use in combination with any of the disclosures provided herein.

### BRIEF DESCRIPTION OF THE DRAWINGS

FIG. 1: Exemplary Gen 2 (process 2A) chart providing an overview of Steps A through F.

FIG. 2A-2C: Process flow chart of an embodiment of Gen 2 (process 2A) for TIL manufacturing.

FIG. 3: Shows a diagram of an embodiment of a cryopreserved TIL exemplary manufacturing process (˜22 days).

FIG. 4: Shows a diagram of an embodiment of process 2A, a 22-day process for TIL manufacturing.

FIG. 5: Comparison table of Steps A through F from exemplary embodiments of process 1C and Gen 2 (process 2A) for TIL manufacturing.

FIG. 6: Detailed comparison of an embodiment of process 1C and an embodiment of Gen 2 (process 2A) for TIL manufacturing.

FIG. 7: Exemplary Gen 3 type TIL manufacturing process.

FIG. 8A-8D: A) Shows a comparison between the 2A process (approximately 22-day process) and an embodiment of the Gen 3 process for TIL manufacturing (approximately 14-days to 16-days process). B) Exemplary Process Gen 3 chart providing an overview of Steps A through F (approximately 14-days to 16-days process). C) Chart providing three exemplary Gen 3 processes with an overview of Steps A through F (approximately 14-days to 16-days process) for each of the three process variations. D) Exemplary modified Gen 2-like process providing an overview of Steps A through F (approximately 22-days process).

FIG. 9: Provides an experimental flow chart for comparability between Gen 2 (process 2A) versus Gen 3 processes.

FIG. 10: Shows a comparison between various Gen 2 (process 2A) and the Gen 3.1 process embodiment.

FIG. 11: Table describing various features of embodiments of the Gen 2, Gen 2.1 and Gen 3.0 process.

FIG. 12: Overview of the media conditions for an embodiment of the Gen 3 process, referred to as Gen 3.1.

FIG. 13: Table describing various features of embodiments of the Gen 2, Gen 2.1 and Gen 3.0 process.

FIG. 14: Table comparing various features of embodiments of the Gen 2 and Gen 3.0 processes.

FIG. 15: Table providing media uses in the various embodiments of the described expansion processes.

FIG. 16: Schematic of an exemplary embodiment of the Gen 3 process (a 16-day process).

FIG. 17: Schematic of an exemplary embodiment of a method for expanding T cells from hematopoietic malignancies using Gen 3 expansion platform.

FIG. 18: Provides the structures I-A and I-B. The cylinders refer to individual polypeptide binding domains. Structures I-A and I-B comprise three linearly-linked TNFRSF binding domains derived from e.g., 4-1BBL or an antibody that binds 4-1BB, which fold to form a trivalent protein, which is then linked to a second trivalent protein through IgG1-Fc (including CH3 and CH2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a V_{H} and a V_{L} chain connected by a linker that may comprise hydrophilic residues and Gly and Ser sequences for flexibility, as well as Glu and Lys for solubility.

FIG. 19: Schematic of an exemplary embodiment of the Gen 3 process (a 16-day process).

FIG. 20: Provides a process overview for an exemplary embodiment of the Gen 3.1 process (a 16 day process).

FIG. 21: Schematic of an exemplary embodiment of the Gen 3.1 process (a 16-17 day process).

FIG. 22: Schematic of an exemplary embodiment of the Gen 3 process (a 16-day process).

FIG. 23: Comparison table for exemplary Gen 2 and exemplary Gen 3 processes.

FIG. 24: Schematic of an exemplary embodiment of the Gen 3 process (a 16-17 day process) showing a preparation timeline.

FIG. 25: Schematic of an exemplary embodiment of the Gen 3 process (a 14-16 day process).

FIG. 26A-26B: Schematic of an exemplary embodiment of the Gen 3 process (a 16 day process).

FIG. 27: Schematic of an exemplary embodiment of the Gen 3 process (a 16 day process).

FIG. 28: Comparison of Gen 2, Gen 2.1 and an embodiment of the Gen 3 process (a 16 day process).

FIG. 29: Comparison of Gen 2, Gen 2.1 and an embodiment of the Gen 3 process (a 16 day process).

FIG. 30: Gen 3 embodiment components.

FIG. 31: Gen 3 embodiment flow chart comparison (Gen 3.0, Gen 3.1 control, Gen 3.1 test).

FIG. 32: Shown are the components of an exemplary embodiment of the Gen 3 process (a 16-17 day process).

FIG. 33: Acceptance criteria table.

FIG. 34: Diagram of an exemplary scFv CCR construct.

FIG. 35: Exemplary PD-1 switch CCR designs.

FIG. 36: Exemplary PD-1 switch CCR designs with alternative CD28 signaling domains.

FIG. 37: Exemplary CCR construct designs.

FIG. 38: Exemplary vector design for lentiviral expression of CCRs in TILs for an anti-TROP-2 (V_{L}-linker-V_{H}) CCR including a IgG4 hinge and transmembrane domain and an IL-2Rβ intracellular domain and an embodiment of the present invention.

FIG. 39: Exemplary vector design for lentiviral expression of CCRs in TILs for an anti-FAP (V_{L}-linker-V_{H}) CCR including a CD8α hinge and transmembrane domain and an IL-2R3 intracellular domain and an embodiment of the present invention.

FIG. 40: Exemplary vector design for lentiviral expression of CCRs in TILs for an anti-PD-L1 (V_{L}-linker-V_{H}) CCR using the 38A1 antibody including a CD8α hinge and transmembrane domain and an IL-2Rβ intracellular domain and an embodiment of the present invention.

FIG. 41: Exemplary vector design for retroviral expression of CXCR1 in TILs and an embodiment of the present invention.

FIG. 42: Exemplary vector design for retroviral expression of CCR8 in TILs and an embodiment of the present invention.

FIG. 43: Flow cytometry analysis of a cervical cancer tumor digest. EPCAM phycoerythrin (PE)/TROP-2 PE.

FIG. 44: Flow cytometry analysis of a cervical cancer tumor digest for EPCAM allophycocyanin (APC)/TROP-2 PE.

FIG. 45: EPCAM/TROP-2 expression on a head and neck squamous cell cancer digest.

FIG. 46: EPCAM/TROP-2 expression on a non-small-cell lung cancer tumor digest.

FIG. 47: Cell frequency distribution in TIL preparations from Gen 2 REP preparations. Nine different TILs were thawed and stained for characterization on two different days and PBMCs were used as controls.

FIG. 48: Cell frequency distribution in TIL preparations from Gen 2 REP preparations. Nine different TILs were thawed and stained for characterization on two different days and PBMCs were used as controls.

FIG. 49: Flow cytometry results showing chemokine receptors on CD8⁺ TILs.

FIG. 50: Flow cytometry results showing chemokine receptors on CD4⁺ TILs.

FIG. 51: Exemplary embodiments of chimeric costimulatory receptors of the present invention. Six CCR constructs using PD-1 or anti-PD-1 (38A1) scFv extracellular domains (ECDs) are shown. TM refers to the transmembrane domain and ICN refers to the intracellular domain.

FIG. 52: Map of pQCXIX vector backbone, which is an embodiment of different CCR and chemokine receptor vectors of the present invention.

FIG. 53: Expression of CCR constructs “CCR4” and “CCR5” (as given in FIG. 51) in HEK reporter cells.

FIG. 54: Exemplary embodiments of chimeric costimulatory receptors of the present invention.

FIG. 55: Domain map of the amino acid sequence for two CCRs comprising SP-(38A1 scFv)-(CD28 hinge and transmembrane)-(IL-2Rβ intracellular)-T2A-SP-(19H9 scFv)-(CD28 hinge and transmembrane)-(IL-2Rγ intracellular), using both the 38A1 and 19H9 PD-L1 domains described herein (SEQ ID NO:658).

FIG. 56: Domain map of the amino acid sequence for two CCRs comprising SP-(38A1 scFv)-(CD28 hinge and transmembrane)-(IL-18R1 intracellular)-T2A-SP-(19H9 scFv)-(CD28 hinge and transmembrane)-(IL-18RAP intracellular), using both the 38A1 and 19H9 PD-L1 domains described herein (SEQ ID NO:659).

FIG. 57: Domain map of the amino acid sequence for two CCRs comprising SP-(anti-TROP-2 scFv)-(CD8 hinge)-(IL-2Rβ transmembrane and intracellular)-T2A-SP-(anti-TROP-2 scFv)-(CD8 hinge)-(IL-2Rγ transmembrane and intracellular) (SEQ ID NO:660).

FIG. 58: Domain map of the amino acid sequence for two CCRs comprising SP-(anti-TROP-2 scFv)-(CD8 hinge)-(IL-18R1-transmembrane and intracellular)-T2A-SP-(anti-TROP-2 scFv)-(CD8 hinge)-(IL-18RAP-transmembrane and intracellular) (SEQ ID NO:661).

FIG. 59: Domain map of the amino acid sequence is an amino acid sequence for two CCRs comprising SP-(cAR47A6.4 scFv)-(CD28 hinge-transmembrane)-(IL-2Rβ intracellular)-T2A-SP-(KM4097 scFv)-(CD28 hinge and transmembrane)-(IL-2Rγ intracellular) (SEQ ID NO:662).

FIG. 60: Domain map of the amino acid sequence an amino acid sequence for two CCRs comprising SP-(cAR47A6.4 scFv)-(CD28 hinge-transmembrane)-(IL-18R1 intracellular)-T2A-SP-(KM4097scFv)-(CD28 hinge-transmembrane)-(IL-18RAP intracellular) (SEQ ID NO:663).

FIG. 61: Map of pLenti vector, which is an embodiment of different CCR and chemokine receptor vectors of the present invention.

FIG. 62: (A) Results for HEK-IL-18 reporter cells transduced with biepitope CCR8 (38A1scFv-CD28TM-IL-18R1-T2A-19H9scFv-CD28TM-IL-18RAP) and incubated with biotin conjugated PD-L1 protein after streptavidin-fluorescent staining, and (B) results for HEK-IL-18 reporter cells transduced with CCR12 (cAR47A6.4 scFv-CD28TM-IL-18R1-T2A-KM4097scFv-CD28TM-IL-18RAP) and incubated with biotin conjugated TROP2 protein after streptavidin-fluorescent staining. Expression of both CCR8 and CCR12 is demonstrated by these results.

FIG. 63: hPD-L1 Raji cells were incubated with indicated (x-axis) concentrations of 38A1-IgG4-HA (hemagglutinin) antibody targeting PD-L1 in the presence of competitive hPD-L1 binding antibody 19H9. After 2 hours of incubation, cells were washed and stained with anti-HA-APC (allophycocyanin). The x-axis shows the concentration of the titrated 38A1-IgG4-HA antibody and the Y-axis shows the % PD-L1 positive staining cells of total hPD-L1 Raji cells.

FIG. 64: hPD-L1 Raji cells were incubated with indicated (x-axis) concentrations of 19H9-IgG4-Flag antibody targeting PD-L1 in the presence of competitive hPD-L1 binding antibody 38A1. After 2 hours of incubation, cells were washed and stained with anti-Flag-AF488. In FIG. 64, the x-axis shows the concentration of the titrated 19H9-IgG4-Flag antibody and the y-axis shows % PD-L1 positive staining cells of total hPD-L1 Raji cells.

FIG. 65: Flow cytometry results with staining of the indicated antibody on each axis.

FIG. 66: Effects of AKT inhibitor (AKTi) treatment on TIL expansion and viability at two different concentrations of the pan-AKT inhibitor ipatasertib (0.3 μM and 1 μM) added either during the pre-REP and REP (blue bars) or during the REP stage only (purple bars). Fold expansion and viability of TIL at the end of the 22-day expansion process are shown. Frequency of CD8⁺, CD4⁺ and CD4⁺ (Foxp3⁺) cells after the expansion process on cryopreserved cells are also shown.

FIG. 67: Experimental design to assess the blocking efficacy of the two PD-L1 antibodies (38A1 and 19H9).

FIG. 68: Results of experiments to assess the blocking efficacy of the two PD-L1 antibodies (38A1 and 19H9).

FIG. 69: T-cell subsets of control and AKT inhibitor (AKTi) treated TILs. Frequency of T_{CM} (CD45RA⁻CCR7⁺), TEM (CD45RA⁻CCR7⁻) and T_{EMRA} (CD45⁺ CCR7⁻) cells in CD8⁺ and CD4⁺ TIL after treatment are shown, with * indicating a p<0.05.

FIG. 70: Cytokine and chemokine receptor expression on control and AKT inhibitor (AKTi)-treated TILs. Cryopreserved control or AKTi treated TILs were analyzed by flow cytometry. Representative histogram and frequencies of of IL-7R⁺ and CXCR3⁺ CD8⁺ TILs, and * indicates p<0.05 and ** indicates p<0.01.

FIG. 71: Distribution of CD69 and CD39 single and double positive populations in control and AKT inhibitor (AKTi) treated CD8⁺ TILs as assessed by flow cytometry, with * indicating p<0.05, ** indicating p<0.01, and *** indicating p<0.001.

FIG. 72: Expression of inhibitory receptors and transcription factors on CD69⁻CD39⁻ and CD69⁺ CD39⁺ CD8⁺ TILs; frequency of PD1, LAG-3, TIM-3, and TIGIT as well as Tbet, Eomes, BATF and TOX on CD69⁻CD39⁻ and CD69⁺ CD39⁺ cells, with * indicating p<0.05, ** indicating p<0.01, and **** indicating p<0.0001. A representative histogram and frequency of CD62L expression on CD69⁻CD39⁻ and CD69⁺ CD39⁺ CD8⁺ TIL is shown.

FIG. 73: Marker expression in control and AKT inhibitor (AKTi) treated TILs following overnight stimulation. Cryopreserved control and TILs treated at both pre-REP and REP with 1 μM AKTi were stimulated overnight with anti-CD3/CD28 beads at a bead-to-cell ratio of 1:5. Frequency of CD69⁻CD39⁻ and CD69⁺ CD39⁺ cells and transcription factor expression on CD8⁺ TIL, with * indicating p<0.05, ** indicating p<0.01, and *** indicating p<0.001.

FIG. 74: Cytokine expression on control and AKT inhibitor (AKTi)-treated CD8⁺ TILs, with * indicating p<0.05.

FIG. 75: Results of an allogeneic cytotoxicity assay. On the left panel, results are shown for cryopreserved control and TILs treated during both pre-REP and REP with 1 uM of AKT inhibitor (ipatasertib) cocultured for 24 hours with KILR® THP-1 cells (Eurofins DiscoverX, Fremont, CA, USA) at a 10:1 effector-to-target cell ratio to measure cytotoxicity in an allogeneic setting. The right panel shows results from control and AKT inhibitor (AKTi) treated TILs that were stimulated every 5 days with anti-CD3/CD28 beads at a 1:1 bead-to-cell ratio. Three days after the third stimulation, cells were washed, beads removed, and cells cocultured at a 10:1 effector-to-target cell ratio with KILR THP-1 cells for 24 hours.

FIG. 76: Expansion, viability, and T-cell distribution data for control TILs (gray bars) and decitabine-treated TILs with increasing concentrations of decitabine are shown. Treatment was added either during the REP stage only (blue bars) or during both pre-REP and REP stages (green bars). Panel A shows fold-expansion and viability of TILs at the end of the 22-day expansion process. Panel B shows the frequency of CD8⁺, CD4⁺, and CD4⁺ (Foxp3+) cells by flow cytometry after the expansion process on cryopreserved cells. *P<0.05, **P<0.01.

FIG. 77: T-cell subsets in control and decitabine-treated TILs. Frequency of T_{CM} (CD45RA⁻CCR7⁺), T_{EM} (CD45RA⁻CCR7⁻), and T_{EMRA} (CD45⁺ CCR7⁻) cells is shown in panel A (CD8⁺) and panel B (CD4⁺) TILs after expansion. *P<0.05, **P<0.01.

FIG. 78: Expression of surface markers on decitabine-treated TILs. Control cryopreserved TILs or decitabine-treated cryopreserved TILs were thawed and stained for flow cytometry analysis. Panel A shows expression of CD25, ICOS, CD28, and IL-7R on CD8⁺ TILs. Panel B shows expression of inhibitory receptors PD-1 and TIGIT on CD8⁺ TIL. Similar results were observed for CD4⁺ TIL. *P<0.05, **P<0.01, ***P<0.001, ****P<0.0001.

FIG. 79: Expression of transcription factors in decitabine-treated TILs. Control or decitabine treated cryopreserved TILs were thawed and stained for flow cytometry analysis. Expression of Eomes, KLF2, BATF, and T-bet on CD8⁺ TILs are shown. *P<0.05, **P<0.01.

FIG. 80: Cytokine expression in control and decitabine-treated TILs following in vitro stimulation. Cryopreserved control and decitabine-treated TILs were stimulated overnight with anti-CD3/CD28 beads at a bead-to-cell ratio of 1:5. Expression of IFN-γ (IFNγ), TNF-α (TNFα), and granzyme B (GZMB) on CD8⁺ TILs are shown. *P<0.05, **P<0.01.

FIG. 81: Cytotoxicity of control and decitabine-treated TILs. In panel A, cryopreserved control TILs and TILs treated at REP with 100 nM DAC were cocultured for 24 h with KILR® THP-1 cells (Eurofins DiscoverX, Fremont, CA, USA) at a 10:1 effector:target cell ratio to measure cytotoxicity in an allogeneic setting. In panel B, control and decitabine-treated TILs were stimulated every 5 days with TransActtm (Miltenyi Biotec, Germany). One day after the third stimulation, cells were washed and cocultured at a 10:1 effector-to-target cell ratio with KILR THP-1 cells for 24 h to measure cytotoxicity. *P<0.05.

FIG. 82: Control TILs and decitabine-treated TILs were stimulated every 5 days with TransActtm (Miltenyi Biotec, Germany). One day after the third stimulation, cells were washed and stained for flow cytometry analysis. Expression of IL-7R, PD-1, and TIM3 in TILs after repeated stimulation are shown in panel A, and expression levels of transcription factors in TILs after repeated stimulation are shown in panel B. *P<0.05, **P<0.01.

FIG. 83: Vector design using the pLenti backbone for the CCR7.2 biepitope CCR targeting PD-L1, which is also an embodiment of different CCR and chemokine receptor vectors of the present invention.

FIG. 84: Vector design using the pLenti backbone for the CCR8.2 biepitope CCR targeting PD-L1, which is also an embodiment of different CCR and chemokine receptor vectors of the present invention.

FIG. 85: Vector design using the pLenti backbone for the CCR11.2 biepitope CCR targeting TROP-2, which is also an embodiment of different CCR and chemokine receptor vectors of the present invention.

FIG. 86: Vector design using the pLenti backbone for the CCR12.2 biepitope CCR targeting TROP-2, which is also an embodiment of different CCR and chemokine receptor vectors of the present invention.

FIG. 87: Results from HeKIL-18 reporter line experiments using PD-L1 targeted CCRs (CCR8 and CCR8.2), showing enhanced IL-18 signaling by use of alternate transmembrane (TM) domains. CCR8 is a biepitope CCR with the general structure 38A1scFv-CD28TM-IL-18R1-IC-T2A-19H9scFv-CD28TM-IL-18RAP-IC, as described herein. CCR8.2 is a biepitope CCR with the general structure 38A1scFv-IL-18R1TM-IL-18R1-IC-T2A-19H9scFV-IL-18RAP™-IL-18RAP-IC.

FIG. 88: Results from HeKIL-18 reporter line experiments using PD-L1 targeted CCRs (CCR12 and CCR12.2), showing enhanced IL-18 signaling by use of alternate transmembrane (TM) domains. CCR12 is a biepitope CCR with the general structure cAR47A6.4 scFv-CD28TM-IL-18R1-IC-T2A-KM4097scFv-CD28TM-IL-18RAP-IC, as described herein. CCR12.2 is a biepitope CCR with the general structure cAR47A6.4 scFv-IL-18R1TM-IL-18R1-IC-T2A-KM4097scFv-IL-18RAPTM-IL-18RAP-IC.

FIG. 89: Results of the PD-L1 targeted CCR experiments (as in FIG. 87) shown in comparison to an IL-18 control at different concentrations.

FIG. 90: Results of the TROP-2 targeted CCR experiments (as in FIG. 88) shown in comparison to an IL-18 control at different concentrations.

FIG. 91: Exemplary CCR designs, primarily for constructs with 4-1BB (CD137) intracellular domains, which are also embodiments of the present invention. EC refers to extracellular, TM refers to transmembrane, SP refers to signal peptide, and IC refers to intracellular.

FIG. 92: Vector design using the pLenti backbone for the CCR13 CCR targeting FAS, which is also an embodiment of different CCR and chemokine receptor vectors of the present invention.

FIG. 93: Vector design using the pLenti backbone for the CCR14 CCR targeting PD-1, which is also an embodiment of different CCR and chemokine receptor vectors of the present invention.

FIG. 94: Vector design using the pLenti backbone for the CCR15 CCR targeting TGFβRII, which is also an embodiment of different CCR and chemokine receptor vectors of the present invention.

FIG. 95: Vector design using the pLenti backbone for the CCR14 CCR targeting PD-1 (with a CD28 intracellular domain), which is also an embodiment of different CCR and chemokine receptor vectors of the present invention.

FIG. 96: CCR constructs were inserted into pLenti-IRES-GFP lentiviral plasmid. TILs were transduced by lentivirus, rested 2 days, then expanded using an 11 day REP expansion process. Surface expression of CCR constructs shown here was detected by flow cytometry.

FIG. 97: Expansion, viability and killing efficacy of CCR-expressing post-REP TILs.

FIG. 98: Exemplary CCR designs for constructs with LTBR intracellular domains, which are also embodiments of the present invention. EC refers to extracellular, TM refers to transmembrane, SP refers to signal peptide, and IC refers to intracellular.

FIG. 99: Vector design using the pLenti backbone for the CCR17 CCR targeting FAS, which is also an embodiment of different CCR and chemokine receptor vectors of the present invention.

FIG. 100: Vector design using the pLenti backbone for the CCR18 CCR targeting PD-1, which is also an embodiment of different CCR and chemokine receptor vectors of the present invention.

FIG. 101: Vector design using the pLenti backbone for the CCR19 CCR targeting TGFβRII, which is also an embodiment of different CCR and chemokine receptor vectors of the present invention.

FIG. 102: Table listing exemplary amino acid sequences for DLL3 antibody variable light chain region (VL1-VL92) comprising FR1, CDR1, FR2, CDR2, FR3, and CDR3 (SEQ ID NOs: 724-815).

FIG. 103: Table listing exemplary amino acid sequences for humanized DLL3 antibody variable light chain region (VL93-VL97) comprising FR1, CDR1, FR2, CDR2, FR3, and CDR3 (SEQ ID NOs: 816-820).

FIG. 104: Table listing exemplary amino acid sequences for DLL3 antibody variable heavy chain region (VH1-VH92 in respective order to their corresponding VL1-VL92 provided in FIG. 102) comprising FR1, CDR1, FR2, CDR2, FR3, and CDR3 (SEQ ID NOs: 821-912).

FIG. 105: Table listing exemplary amino acid sequence for humanized DLL3 antibody variable heavy chain region (VH93-VH97 in respective order to their corresponding VL93-VL97 provided in FIG. 103) comprising FR1, CDR1, FR2, CDR2, FR3, and CDR3 (SEQ ID NOs: 913-917).

### BRIEF DESCRIPTION OF THE SEQUENCE LISTING

SEQ ID NO:1 is the amino acid sequence of the heavy chain of muromonab.

SEQ ID NO:2 is the amino acid sequence of the light chain of muromonab.

SEQ ID NO:3 is the amino acid sequence of a recombinant human IL-2 protein.

SEQ ID NO:4 is the amino acid sequence of aldesleukin.

SEQ ID NO:5 is an IL-2 form.

SEQ ID NO:6 is the amino acid sequence of nemvaleukin alfa.

SEQ ID NO:7 is an IL-2 form.

SEQ ID NO:8 is a mucin domain polypeptide.

SEQ ID NO:9 is the amino acid sequence of a recombinant human IL-4 protein.

SEQ ID NO:10 is the amino acid sequence of a recombinant human IL-7 protein.

SEQ ID NO: 11 is the amino acid sequence of a recombinant human IL-15 protein.

SEQ ID NO: 12 is the amino acid sequence of a recombinant human IL-21 protein.

SEQ ID NO: 13 is an IL-2 sequence.

SEQ ID NO: 14 is an IL-2 mutein sequence.

SEQ ID NO:15 is an IL-2 mutein sequence.

SEQ ID NO:16 is the HCDR1_IL-2 for IgG.IL2R67A.H1.

SEQ ID NO: 17 is the HCDR2 for IgG.IL2R67A.H1.

SEQ ID NO: 18 is the HCDR3 for IgG.IL2R67A.H1.

SEQ ID NO: 19 is the HCDR1_IL-2 kabat for IgG.IL2R67A.H1.

SEQ ID NO:20 is the HCDR2 kabat for IgG.IL2R67A.H1.

SEQ ID NO:21 is the HCDR3 kabat for IgG.IL2R67A.H1.

SEQ ID NO:22 is the HCDR1_IL-2 clothia for IgG.IL2R67A.H1.

SEQ ID NO:23 is the HCDR2 clothia for IgG.IL2R67A.H1.

SEQ ID NO:24 is the HCDR3 clothia for IgG.IL2R67A.H1.

SEQ ID NO:25 is the HCDR1_IL-2 IMGT for IgG.IL2R67A.H1.

SEQ ID NO:26 is the HCDR2 IMGT for IgG.IL2R67A.H1.

SEQ ID NO:27 is the HCDR3 IMGT for IgG.IL2R67A.H1.

SEQ ID NO:28 is the V_{H} chain for IgG.IL2R67A.H1.

SEQ ID NO:29 is the heavy chain for IgG.IL2R67A.H1.

SEQ ID NO:30 is the LCDR1 kabat for IgG.IL2R67A.H1.

SEQ ID NO:31 is the LCDR2 kabat for IgG.IL2R67A.H1.

SEQ ID NO:32 is the LCDR3 kabat for IgG.IL2R67A.H1.

SEQ ID NO: 33 is the LCDR1 chothia for IgG.IL2R67A.H1.

SEQ ID NO: 34 is the LCDR2 chothia for IgG.IL2R67A.H1.

SEQ ID NO: 35 is the LCDR3 chothia for IgG.IL2R67A.H1.

SEQ ID NO: 36 is a V_{L} chain.

SEQ ID NO: 37 is a light chain.

SEQ ID NO:38 is a light chain.

SEQ ID NO:39 is a light chain.

SEQ ID NO:40 is the amino acid sequence of human 4-1BB.

SEQ ID NO:41 is the amino acid sequence of murine 4-1BB.

SEQ ID NO:42 is the heavy chain for the 4-1BB agonist monoclonal antibody utomilumab (PF-05082566).

SEQ ID NO:43 is the light chain for the 4-1BB agonist monoclonal antibody utomilumab (PF-05082566).

SEQ ID NO:44 is the heavy chain variable region (V_{H}) for the 4-1BB agonist monoclonal antibody utomilumab (PF-05082566).

SEQ ID NO:45 is the light chain variable region (V_{L}) for the 4-1BB agonist monoclonal antibody utomilumab (PF-05082566).

SEQ ID NO:46 is the heavy chain CDR1 for the 4-1BB agonist monoclonal antibody utomilumab (PF-05082566).

SEQ ID NO:47 is the heavy chain CDR2 for the 4-1BB agonist monoclonal antibody utomilumab (PF-05082566).

SEQ ID NO:48 is the heavy chain CDR3 for the 4-1BB agonist monoclonal antibody utomilumab (PF-05082566).

SEQ ID NO:49 is the light chain CDR1 for the 4-1BB agonist monoclonal antibody utomilumab (PF-05082566).

SEQ ID NO:50 is the light chain CDR2 for the 4-1BB agonist monoclonal antibody utomilumab (PF-05082566).

SEQ ID NO:51 is the light chain CDR3 for the 4-1BB agonist monoclonal antibody utomilumab (PF-05082566).

SEQ ID NO:52 is the heavy chain for the 4-1BB agonist monoclonal antibody urelumab (BMS-663513).

SEQ ID NO:53 is the light chain for the 4-1BB agonist monoclonal antibody urelumab (BMS-663513).

SEQ ID NO:54 is the heavy chain variable region (VH) for the 4-1BB agonist monoclonal antibody urelumab (BMS-663513).

SEQ ID NO:55 is the light chain variable region (VL) for the 4-1BB agonist monoclonal antibody urelumab (BMS-663513).

SEQ ID NO:56 is the heavy chain CDR1 for the 4-1BB agonist monoclonal antibody urelumab (BMS-663513).

SEQ ID NO:57 is the heavy chain CDR2 for the 4-1BB agonist monoclonal antibody urelumab (BMS-663513).

SEQ ID NO:58 is the heavy chain CDR3 for the 4-1BB agonist monoclonal antibody urelumab (BMS-663513).

SEQ ID NO:59 is the light chain CDR1 for the 4-1BB agonist monoclonal antibody urelumab (BMS-663513).

SEQ ID NO:60 is the light chain CDR2 for the 4-1BB agonist monoclonal antibody urelumab (BMS-663513).

SEQ ID NO:61 is the light chain CDR3 for the 4-1BB agonist monoclonal antibody urelumab (BMS-663513).

SEQ ID NO:62 is an Fc domain for a TNFRSF agonist fusion protein.

SEQ ID NO: 63 is a linker for a TNFRSF agonist fusion protein or for an scFv.

SEQ ID NO: 64 is a linker for a TNFRSF agonist fusion protein or for an scFv.

SEQ ID NO: 65 is a linker for a TNFRSF agonist fusion protein or for an scFv.

SEQ ID NO: 66 is a linker for a TNFRSF agonist fusion protein or for an scFv.

SEQ ID NO: 67 is a linker for a TNFRSF agonist fusion protein or for an scFv.

SEQ ID NO: 68 is a linker for a TNFRSF agonist fusion protein or for an scFv.

SEQ ID NO: 69 is a linker for a TNFRSF agonist fusion protein or for an scFv.

SEQ ID NO: 70 is a linker for a TNFRSF agonist fusion protein or for an scFv.

SEQ ID NO:71 is a linker for a TNFRSF agonist fusion protein or for an scFv.

SEQ ID NO: 72 is a linker for a TNFRSF agonist fusion protein or for an scFv.

SEQ ID NO: 73 is an Fc domain for a TNFRSF agonist fusion protein.

SEQ ID NO: 74 is a linker for a TNFRSF agonist fusion protein or for an scFv.

SEQ ID NO: 75 is a linker for a TNFRSF agonist fusion protein or for an scFv.

SEQ ID NO:76 is a linker for a TNFRSF agonist fusion protein or for an scFv.

SEQ ID NO:77 is a 4-1BB ligand (4-1BBL) amino acid sequence.

SEQ ID NO:78 is a soluble portion of 4-1BBL polypeptide.

SEQ ID NO:79 is a heavy chain variable region (V_{H}) for the 4-1BB agonist antibody 4B4-1-1 version 1.

SEQ ID NO:80 is a light chain variable region (V_{L}) for the 4-1BB agonist antibody 4B4-1-1 version 1.

SEQ ID NO:81 is a heavy chain variable region (V_{H}) for the 4-1BB agonist antibody 4B4-1-1 version 2.

SEQ ID NO:82 is a light chain variable region (V_{L}) for the 4-1BB agonist antibody 4B4-1-1 version 2.

SEQ ID NO:83 is a heavy chain variable region (V_{H}) for the 4-1BB agonist antibody H39E3-2.

SEQ ID NO:84 is a light chain variable region (V_{L}) for the 4-1BB agonist antibody H39E3-2.

SEQ ID NO: 85 is the amino acid sequence of human OX40.

SEQ ID NO: 86 is the amino acid sequence of murine OX40.

SEQ ID NO: 87 is the heavy chain for the OX40 agonist monoclonal antibody tavolixizumab (MEDI-0562).

SEQ ID NO:88 is the light chain for the OX40 agonist monoclonal antibody tavolixizumab (MEDI-0562).

SEQ ID NO:89 is the heavy chain variable region (V_{H}) for the OX40 agonist monoclonal antibody tavolixizumab (MEDI-0562).

SEQ ID NO:90 is the light chain variable region (V_{L}) for the OX40 agonist monoclonal antibody tavolixizumab (MEDI-0562).

SEQ ID NO:91 is the heavy chain CDR1 for the OX40 agonist monoclonal antibody tavolixizumab (MEDI-0562).

SEQ ID NO:92 is the heavy chain CDR2 for the OX40 agonist monoclonal antibody tavolixizumab (MEDI-0562).

SEQ ID NO:93 is the heavy chain CDR3 for the OX40 agonist monoclonal antibody tavolixizumab (MEDI-0562).

SEQ ID NO:94 is the light chain CDR1 for the OX40 agonist monoclonal antibody tavolixizumab (MEDI-0562).

SEQ ID NO:95 is the light chain CDR2 for the OX40 agonist monoclonal antibody tavolixizumab (MEDI-0562).

SEQ ID NO:96 is the light chain CDR3 for the OX40 agonist monoclonal antibody tavolixizumab (MEDI-0562).

SEQ ID NO:97 is the heavy chain for the OX40 agonist monoclonal antibody 11D4.

SEQ ID NO: 98 is the light chain for the OX40 agonist monoclonal antibody 11D4.

SEQ ID NO:99 is the heavy chain variable region (V_{H}) for the OX40 agonist monoclonal antibody 11D4.

SEQ ID NO:100 is the light chain variable region (V_{L}) for the OX40 agonist monoclonal antibody 11D4.

SEQ ID NO:101 is the heavy chain CDR1 for the OX40 agonist monoclonal antibody 11D4.

SEQ ID NO:102 is the heavy chain CDR2 for the OX40 agonist monoclonal antibody 11D4.

SEQ ID NO:103 is the heavy chain CDR3 for the OX40 agonist monoclonal antibody 11D4.

SEQ ID NO:104 is the light chain CDR1 for the OX40 agonist monoclonal antibody 11D4.

SEQ ID NO:105 is the light chain CDR2 for the OX40 agonist monoclonal antibody 11D4.

SEQ ID NO: 106 is the light chain CDR3 for the OX40 agonist monoclonal antibody 11D4.

SEQ ID NO:107 is the heavy chain for the OX40 agonist monoclonal antibody 18D8.

SEQ ID NO:108 is the light chain for the OX40 agonist monoclonal antibody 18D8.

SEQ ID NO:109 is the heavy chain variable region (V_{H}) for the OX40 agonist monoclonal antibody 18D8.

SEQ ID NO:110 is the light chain variable region (V_{L}) for the OX40 agonist monoclonal antibody 18D8.

SEQ ID NO:111 is the heavy chain CDR1 for the OX40 agonist monoclonal antibody 18D8.

SEQ ID NO:112 is the heavy chain CDR2 for the OX40 agonist monoclonal antibody 18D8.

SEQ ID NO:113 is the heavy chain CDR3 for the OX40 agonist monoclonal antibody 18D8.

SEQ ID NO:114 is the light chain CDR1 for the OX40 agonist monoclonal antibody 18D8.

SEQ ID NO:115 is the light chain CDR2 for the OX40 agonist monoclonal antibody 18D8.

SEQ ID NO:116 is the light chain CDR3 for the OX40 agonist monoclonal antibody 18D8.

SEQ ID NO:117 is the heavy chain variable region (V_{H}) for the OX40 agonist monoclonal antibody Hu119-122.

SEQ ID NO:118 is the light chain variable region (V_{L}) for the OX40 agonist monoclonal antibody Hu119-122.

SEQ ID NO:119 is the heavy chain CDR1 for the OX40 agonist monoclonal antibody Hu119-122.

SEQ ID NO: 120 is the heavy chain CDR2 for the OX40 agonist monoclonal antibody Hu119-122.

SEQ ID NO:121 is the heavy chain CDR3 for the OX40 agonist monoclonal antibody Hu119-122.

SEQ ID NO:122 is the light chain CDR1 for the OX40 agonist monoclonal antibody Hu119-122.

SEQ ID NO:123 is the light chain CDR2 for the OX40 agonist monoclonal antibody Hu119-122.

SEQ ID NO:124 is the light chain CDR3 for the OX40 agonist monoclonal antibody Hu119-122.

SEQ ID NO:125 is the heavy chain variable region (V_{H}) for the OX40 agonist monoclonal antibody Hu106-222.

SEQ ID NO:126 is the light chain variable region (V_{L}) for the OX40 agonist monoclonal antibody Hu106-222.

SEQ ID NO:127 is the heavy chain CDR1 for the OX40 agonist monoclonal antibody Hu106-222.

SEQ ID NO:128 is the heavy chain CDR2 for the OX40 agonist monoclonal antibody Hu106-222.

SEQ ID NO:129 is the heavy chain CDR3 for the OX40 agonist monoclonal antibody Hu106-222.

SEQ ID NO:130 is the light chain CDR1 for the OX40 agonist monoclonal antibody Hu106-222.

SEQ ID NO:131 is the light chain CDR2 for the OX40 agonist monoclonal antibody Hu106-222.

SEQ ID NO:132 is the light chain CDR3 for the OX40 agonist monoclonal antibody Hu106-222.

SEQ ID NO:133 is an OX40 ligand (OX40L) amino acid sequence.

SEQ ID NO:134 is a soluble portion of OX40L polypeptide.

SEQ ID NO:135 is an alternative soluble portion of OX40L polypeptide.

SEQ ID NO:136 is the heavy chain variable region (V_{H}) for the OX40 agonist monoclonal antibody 008.

SEQ ID NO:137 is the light chain variable region (V_{L}) for the OX40 agonist monoclonal antibody 008.

SEQ ID NO:138 is the heavy chain variable region (V_{H}) for the OX40 agonist monoclonal antibody 011.

SEQ ID NO:139 is the light chain variable region (V_{L}) for the OX40 agonist monoclonal antibody 011.

SEQ ID NO:140 is the heavy chain variable region (V_{H}) for the OX40 agonist monoclonal antibody 021.

SEQ ID NO: 141 is the light chain variable region (V_{L}) for the OX40 agonist monoclonal antibody 021.

SEQ ID NO:142 is the heavy chain variable region (V_{H}) for the OX40 agonist monoclonal antibody 023.

SEQ ID NO:143 is the light chain variable region (V_{L}) for the OX40 agonist monoclonal antibody 023.

SEQ ID NO:144 is the heavy chain variable region (V_{H}) for an OX40 agonist monoclonal antibody.

SEQ ID NO: 145 is the light chain variable region (V_{L}) for an OX40 agonist monoclonal antibody.

SEQ ID NO:146 is the heavy chain variable region (V_{H}) for an OX40 agonist monoclonal antibody.

SEQ ID NO: 147 is the light chain variable region (V_{L}) for an OX40 agonist monoclonal antibody.

SEQ ID NO:148 is the heavy chain variable region (V_{H}) for a humanized OX40 agonist monoclonal antibody.

SEQ ID NO:149 is the heavy chain variable region (V_{H}) for a humanized OX40 agonist monoclonal antibody.

SEQ ID NO: 150 is the light chain variable region (V_{L}) for a humanized OX40 agonist monoclonal antibody.

SEQ ID NO:151 is the light chain variable region (V_{L}) for a humanized OX40 agonist monoclonal antibody.

SEQ ID NO:152 is the heavy chain variable region (V_{H}) for a humanized OX40 agonist monoclonal antibody.

SEQ ID NO:153 is the heavy chain variable region (V_{H}) for a humanized OX40 agonist monoclonal antibody.

SEQ ID NO:154 is the light chain variable region (V_{L}) for a humanized OX40 agonist monoclonal antibody.

SEQ ID NO:155 is the light chain variable region (V_{L}) for a humanized OX40 agonist monoclonal antibody.

SEQ ID NO:156 is the heavy chain variable region (V_{H}) for an OX40 agonist monoclonal antibody.

SEQ ID NO: 157 is the light chain variable region (V_{L}) for an OX40 agonist monoclonal antibody.

SEQ ID NO: 158 is the heavy chain amino acid sequence of the PD-1 inhibitor nivolumab.

SEQ ID NO:159 is the light chain amino acid sequence of the PD-1 inhibitor nivolumab.

SEQ ID NO:160 is the heavy chain variable region (V_{H}) amino acid sequence of the PD-1 inhibitor nivolumab.

SEQ ID NO:161 is the light chain variable region (V_{L}) amino acid sequence of the PD-1 inhibitor nivolumab.

SEQ ID NO:162 is the heavy chain CDR1 amino acid sequence of the PD-1 inhibitor nivolumab.

SEQ ID NO:163 is the heavy chain CDR2 amino acid sequence of the PD-1 inhibitor nivolumab.

SEQ ID NO: 164 is the heavy chain CDR3 amino acid sequence of the PD-1 inhibitor nivolumab.

SEQ ID NO:165 is the light chain CDR1 amino acid sequence of the PD-1 inhibitor nivolumab.

SEQ ID NO:166 is the light chain CDR2 amino acid sequence of the PD-1 inhibitor nivolumab.

SEQ ID NO:167 is the light chain CDR3 amino acid sequence of the PD-1 inhibitor nivolumab.

SEQ ID NO:168 is the heavy chain amino acid sequence of the PD-1 inhibitor pembrolizumab.

SEQ ID NO:169 is the light chain amino acid sequence of the PD-1 inhibitor pembrolizumab.

SEQ ID NO:170 is the heavy chain variable region (V_{H}) amino acid sequence of the PD-1 inhibitor pembrolizumab.

SEQ ID NO:171 is the light chain variable region (V_{L}) amino acid sequence of the PD-1 inhibitor pembrolizumab.

SEQ ID NO:172 is the heavy chain CDR1 amino acid sequence of the PD-1 inhibitor pembrolizumab.

SEQ ID NO:173 is the heavy chain CDR2 amino acid sequence of the PD-1 inhibitor pembrolizumab.

SEQ ID NO:174 is the heavy chain CDR3 amino acid sequence of the PD-1 inhibitor pembrolizumab.

SEQ ID NO:175 is the light chain CDR1 amino acid sequence of the PD-1 inhibitor pembrolizumab.

SEQ ID NO:176 is the light chain CDR2 amino acid sequence of the PD-1 inhibitor pembrolizumab.

SEQ ID NO:177 is the light chain CDR3 amino acid sequence of the PD-1 inhibitor pembrolizumab.

SEQ ID NO: 178 is the heavy chain amino acid sequence of the PD-L1 inhibitor durvalumab.

SEQ ID NO:179 is the light chain amino acid sequence of the PD-L1 inhibitor durvalumab.

SEQ ID NO:180 is the heavy chain variable region (V_{H}) amino acid sequence of the PD-L1 inhibitor durvalumab.

SEQ ID NO:181 is the light chain variable region (V_{L}) amino acid sequence of the PD-L1 inhibitor durvalumab.

SEQ ID NO:182 is the heavy chain CDR1 amino acid sequence of the PD-L1 inhibitor durvalumab.

SEQ ID NO:183 is the heavy chain CDR2 amino acid sequence of the PD-L1 inhibitor durvalumab.

SEQ ID NO:184 is the heavy chain CDR3 amino acid sequence of the PD-L1 inhibitor durvalumab.

SEQ ID NO:185 is the light chain CDR1 amino acid sequence of the PD-L1 inhibitor durvalumab.

SEQ ID NO:186 is the light chain CDR2 amino acid sequence of the PD-L1 inhibitor durvalumab.

SEQ ID NO:187 is the light chain CDR3 amino acid sequence of the PD-L1 inhibitor durvalumab.

SEQ ID NO:188 is the heavy chain amino acid sequence of the PD-L1 inhibitor avelumab.

SEQ ID NO:189 is the light chain amino acid sequence of the PD-L1 inhibitor avelumab.

SEQ ID NO:190 is the heavy chain variable region (V_{H}) amino acid sequence of the PD-L1 inhibitor avelumab.

SEQ ID NO:191 is the light chain variable region (V_{L}) amino acid sequence of the PD-L1 inhibitor avelumab.

SEQ ID NO: 192 is the heavy chain CDR1 amino acid sequence of the PD-L1 inhibitor avelumab.

SEQ ID NO:193 is the heavy chain CDR2 amino acid sequence of the PD-L1 inhibitor avelumab.

SEQ ID NO:194 is the heavy chain CDR3 amino acid sequence of the PD-L1 inhibitor avelumab.

SEQ ID NO:195 is the light chain CDR1 amino acid sequence of the PD-L1 inhibitor avelumab.

SEQ ID NO:196 is the light chain CDR2 amino acid sequence of the PD-L1 inhibitor avelumab.

SEQ ID NO:197 is the light chain CDR3 amino acid sequence of the PD-L1 inhibitor avelumab.

SEQ ID NO:198 is the heavy chain amino acid sequence of the PD-L1 inhibitor atezolizumab.

SEQ ID NO:199 is the light chain amino acid sequence of the PD-L1 inhibitor atezolizumab.

SEQ ID NO:200 is the heavy chain variable region (V_{H}) amino acid sequence of the PD-L1 inhibitor atezolizumab.

SEQ ID NO:201 is the light chain variable region (V_{L}) amino acid sequence of the PD-L1 inhibitor atezolizumab.

SEQ ID NO:202 is the heavy chain CDR1 amino acid sequence of the PD-L1 inhibitor atezolizumab.

SEQ ID NO:203 is the heavy chain CDR2 amino acid sequence of the PD-L1 inhibitor atezolizumab.

SEQ ID NO:204 is the heavy chain CDR3 amino acid sequence of the PD-L1 inhibitor atezolizumab.

SEQ ID NO:205 is the light chain CDR1 amino acid sequence of the PD-L1 inhibitor atezolizumab.

SEQ ID NO:206 is the light chain CDR2 amino acid sequence of the PD-L1 inhibitor atezolizumab.

SEQ ID NO:207 is the light chain CDR3 amino acid sequence of the PD-L1 inhibitor atezolizumab.

SEQ ID NO:208 is the heavy chain amino acid sequence of the CTLA-4 inhibitor ipilimumab.

SEQ ID NO:209 is the light chain amino acid sequence of the CTLA-4 inhibitor ipilimumab.

SEQ ID NO:210 is the heavy chain variable region (V_{H}) amino acid sequence of the CTLA-4 inhibitor ipilimumab.

SEQ ID NO:211 is the light chain variable region (V_{L}) amino acid sequence of the CTLA-4 inhibitor ipilimumab.

SEQ ID NO:212 is the heavy chain CDR1 amino acid sequence of the CTLA-4 inhibitor ipilimumab.

SEQ ID NO:213 is the heavy chain CDR2 amino acid sequence of the CTLA-4 inhibitor ipilimumab.

SEQ ID NO:214 is the heavy chain CDR3 amino acid sequence of the CTLA-4 inhibitor ipilimumab.

SEQ ID NO:215 is the light chain CDR1 amino acid sequence of the CTLA-4 inhibitor ipilimumab.

SEQ ID NO:216 is the light chain CDR2 amino acid sequence of the CTLA-4 inhibitor ipilimumab.

SEQ ID NO:217 is the light chain CDR3 amino acid sequence of the CTLA-4 inhibitor ipilimumab.

SEQ ID NO:218 is the heavy chain amino acid sequence of the CTLA-4 inhibitor tremelimumab.

SEQ ID NO:219 is the light chain amino acid sequence of the CTLA-4 inhibitor tremelimumab.

SEQ ID NO:220 is the heavy chain variable region (V_{H}) amino acid sequence of the CTLA-4 inhibitor tremelimumab.

SEQ ID NO:221 is the light chain variable region (V_{L}) amino acid sequence of the CTLA-4 inhibitor tremelimumab.

SEQ ID NO:222 is the heavy chain CDR1 amino acid sequence of the CTLA-4 inhibitor tremelimumab.

SEQ ID NO:223 is the heavy chain CDR2 amino acid sequence of the CTLA-4 inhibitor tremelimumab.

SEQ ID NO:224 is the heavy chain CDR3 amino acid sequence of the CTLA-4 inhibitor tremelimumab.

SEQ ID NO:225 is the light chain CDR1 amino acid sequence of the CTLA-4 inhibitor tremelimumab.

SEQ ID NO:226 is the light chain CDR2 amino acid sequence of the CTLA-4 inhibitor tremelimumab.

SEQ ID NO:227 is the light chain CDR3 amino acid sequence of the CTLA-4 inhibitor tremelimumab.

SEQ ID NO:228 is the heavy chain amino acid sequence of the CTLA-4 inhibitor zalifrelimab.

SEQ ID NO:229 is the light chain amino acid sequence of the CTLA-4 inhibitor zalifrelimab.

SEQ ID NO:230 is the heavy chain variable region (V_{H}) amino acid sequence of the CTLA-4 inhibitor zalifrelimab.

SEQ ID NO:231 is the light chain variable region (V_{L}) amino acid sequence of the CTLA-4 inhibitor zalifrelimab.

SEQ ID NO:232 is the heavy chain CDR1 amino acid sequence of the CTLA-4 inhibitor zalifrelimab.

SEQ ID NO:233 is the heavy chain CDR2 amino acid sequence of the CTLA-4 inhibitor zalifrelimab.

SEQ ID NO:234 is the heavy chain CDR3 amino acid sequence of the CTLA-4 inhibitor zalifrelimab.

SEQ ID NO:235 is the light chain CDR1 amino acid sequence of the CTLA-4 inhibitor zalifrelimab.

SEQ ID NO:236 is the light chain CDR2 amino acid sequence of the CTLA-4 inhibitor zalifrelimab.

SEQ ID NO:237 is the light chain CDR3 amino acid sequence of the CTLA-4 inhibitor zalifrelimab.

SEQ ID NO:238 is the amino acid sequence of an scFv linker.

SEQ ID NO:239 is the amino acid sequence of an scFv linker.

SEQ ID NO:240 is the amino acid sequence of an scFv linker.

SEQ ID NO:241 is the amino acid sequence of an scFv linker.

SEQ ID NO:242 is the amino acid sequence of an scFv linker.

SEQ ID NO:243 is the amino acid sequence of an scFv linker.

SEQ ID NO:244 is the amino acid sequence of a PD-1 extracellular domain.

SEQ ID NO:245 is the amino acid sequence of a PD-1 extracellular and transmembrane domain.

SEQ ID NO:246 is the amino acid sequence of a PD-1 extracellular domain and CD28 transmembrane domain.

SEQ ID NO:247 is the nucleotide sequence of a PD-1 extracellular and transmembrane domain.

SEQ ID NO:248 is the nucleotide sequence of a PD-1 extracellular domain and CD28 transmembrane domain.

SEQ ID NO:249 is the amino acid sequence of scFv-Fc antibody 38A1.

SEQ ID NO:250 is the amino acid sequence of scFv antibody 38A1 variable heavy chain.

SEQ ID NO:251 is the amino acid sequence of scFv antibody 38A1 variable light chain.

SEQ ID NO:252 is the amino acid sequence of scFv antibody 38A1 variable heavy chain CDR1.

SEQ ID NO:253 is the amino acid sequence of scFv antibody 38A1 variable heavy chain CDR2.

SEQ ID NO:254 is the amino acid sequence of scFv antibody 38A1 variable heavy chain CDR3.

SEQ ID NO:255 is the amino acid sequence of scFv antibody 38A1 variable light chain CDR1.

SEQ ID NO:256 is the amino acid sequence of scFv antibody 38A1 variable light chain CDR2.

SEQ ID NO:257 is the amino acid sequence of scFv antibody 38A1 variable light chain CDR3.

SEQ ID NO:258 is the amino acid sequence of scFv-Fc antibody 19H9.

SEQ ID NO:259 is the amino acid sequence of scFv antibody 19H9 variable heavy chain.

SEQ ID NO:260 is the amino acid sequence of scFv antibody 19H9 variable light chain.

SEQ ID NO:261 is the amino acid sequence of scFv antibody 19H9 variable heavy chain CDR1.

SEQ ID NO:262 is the amino acid sequence of scFv antibody 19H9 variable heavy chain CDR2.

SEQ ID NO:263 is the amino acid sequence of scFv antibody 19H9 variable heavy chain CDR3.

SEQ ID NO:264 is the amino acid sequence of scFv antibody 19H9 variable light chain CDR1.

SEQ ID NO:265 is the amino acid sequence of scFv antibody 19H9 variable light chain CDR2.

SEQ ID NO:266 is the amino acid sequence of scFv antibody 19H9 variable light chain CDR3.

SEQ ID NO:267 is an anti-CEA variable heavy chain amino acid sequence.

SEQ ID NO:268 is an anti-CEA variable light chain amino acid sequence.

SEQ ID NO:269 is an anti-CEA heavy chain CDR1 amino acid sequence.

SEQ ID NO:270 is an anti-CEA heavy chain CDR2 amino acid sequence.

SEQ ID NO:271 is an anti-CEA heavy chain CDR3 amino acid sequence.

SEQ ID NO:272 is an anti-CEA light chain CDR1 amino acid sequence.

SEQ ID NO:273 is an anti-CEA light chain CDR2 amino acid sequence.

SEQ ID NO:274 is an anti-CEA light chain CDR3 amino acid sequence.

SEQ ID NO:275 is an anti-CD73 variable heavy chain amino acid sequence.

SEQ ID NO:276 is an anti-CD73 variable light chain amino acid sequence.

SEQ ID NO:277 is an anti-CD73 heavy chain CDR1 amino acid sequence.

SEQ ID NO:278 is an anti-CD73 heavy chain CDR2 amino acid sequence.

SEQ ID NO:279 is an anti-CD73 heavy chain CDR3 amino acid sequence.

SEQ ID NO:280 is an anti-CD73 light chain CDR1 amino acid sequence.

SEQ ID NO:281 is an anti-CD73 light chain CDR2 amino acid sequence.

SEQ ID NO:282 is an anti-CD73 light chain CDR3 amino acid sequence.

SEQ ID NO:283 is an anti-CD73 variable heavy chain amino acid sequence.

SEQ ID NO:284 is an anti-CD73 variable light chain amino acid sequence.

SEQ ID NO:285 is an anti-CD73 heavy chain CDR1 amino acid sequence.

SEQ ID NO:286 is an anti-CD73 heavy chain CDR2 amino acid sequence.

SEQ ID NO:287 is an anti-CD73 heavy chain CDR3 amino acid sequence.

SEQ ID NO:288 is an anti-CD73 light chain CDR1 amino acid sequence.

SEQ ID NO:289 is an anti-CD73 light chain CDR2 amino acid sequence.

SEQ ID NO:290 is an anti-CD73 light chain CDR3 amino acid sequence.

SEQ ID NO:291 is an anti-TROP-2 variable heavy chain amino acid sequence.

SEQ ID NO:292 is an anti-TROP-2 variable heavy chain amino acid sequence.

SEQ ID NO:293 is an anti-TROP-2 variable heavy chain amino acid sequence.

SEQ ID NO:294 is an anti-TROP-2 variable heavy chain amino acid sequence.

SEQ ID NO:295 is an anti-TROP-2 variable heavy chain amino acid sequence.

SEQ ID NO:296 is an anti-TROP-2 variable heavy chain amino acid sequence.

SEQ ID NO:297 is an anti-TROP-2 variable light chain amino acid sequence.

SEQ ID NO:298 is an anti-TROP-2 variable light chain amino acid sequence.

SEQ ID NO:299 is an anti-TROP-2 variable light chain amino acid sequence.

SEQ ID NO:300 is an anti-TROP-2 variable light chain amino acid sequence.

SEQ ID NO:301 is an anti-TROP-2 heavy chain CDR1 amino acid sequence.

SEQ ID NO:302 is an anti-TROP-2 heavy chain CDR2 amino acid sequence.

SEQ ID NO:303 is an anti-TROP-2 heavy chain CDR3 amino acid sequence.

SEQ ID NO:304 is an anti-TROP-2 light chain CDR1 amino acid sequence.

SEQ ID NO:305 is an anti-TROP-2 light chain CDR2 amino acid sequence.

SEQ ID NO:306 is an anti-TROP-2 light chain CDR3 amino acid sequence.

SEQ ID NO:307 is the amino acid sequence of anti-TROP-2 antibody m7E6 variable heavy chain.

SEQ ID NO:308 is the amino acid sequence of anti-TROP-2 antibody m7E6 variable light chain.

SEQ ID NO:309 is the amino acid sequence of anti-TROP-2 antibody h7E6 variable heavy chain.

SEQ ID NO:310 is the amino acid sequence of anti-TROP-2 antibody m7E6 and h7E6_SVG variable light chain.

SEQ ID NO:311 is the amino acid sequence of anti-TROP-2 antibody h7E6_SVGL and h7E6_SVG variable heavy chain.

SEQ ID NO:312 is the amino acid sequence of anti-TROP-2 antibody h7E6_SVGL variable light chain.

SEQ ID NO:313 is the amino acid sequence of anti-TROP-2 antibody m6G11 variable heavy chain.

SEQ ID NO:314 is the amino acid sequence of anti-TROP-2 antibody m6G11 variable light chain.

SEQ ID NO:315 is the amino acid sequence of anti-TROP-2 antibody h6G11 variable heavy chain.

SEQ ID NO:316 is the amino acid sequence of anti-TROP-2 antibody h6G11 variable light chain.

SEQ ID NO:317 is the amino acid sequence of anti-TROP-2 antibody h6G11-FKG_SF variable heavy chain.

SEQ ID NO:318 is the amino acid sequence of anti-TROP-2 antibody h6G11-FKG_SF variable light chain.

SEQ ID NO:319 is the amino acid sequence of an anti-TROP-2 antibody variable heavy chain CDR1.

SEQ ID NO:320 is the amino acid sequence of an anti-TROP-2 antibody variable heavy chain CDR2.

SEQ ID NO:321 is the amino acid sequence of an anti-TROP-2 antibody variable heavy chain CDR3.

SEQ ID NO:322 is the amino acid sequence of an anti-TROP-2 antibody variable light chain CDR1.

SEQ ID NO:323 is the amino acid sequence of an anti-TROP-2 antibody variable light chain CDR2.

SEQ ID NO:324 is the amino acid sequence of an anti-TROP-2 antibody variable light chain CDR3.

SEQ ID NO:325 is the nucleotide sequence encoding anti-TROP-2 antibody m7E6 variable heavy chain.

SEQ ID NO:326 is the nucleotide sequence encoding anti-TROP-2 antibody m7E6 variable light chain.

SEQ ID NO:327 is the nucleotide sequence encoding anti-TROP-2 antibody h7E6 variable heavy chain.

SEQ ID NO:328 is the nucleotide sequence encoding anti-TROP-2 antibody m7E6 variable light chain.

SEQ ID NO:329 is the nucleotide sequence encoding anti-TROP-2 antibody h7E6_SVGL variable heavy chain.

SEQ ID NO:330 is the nucleotide sequence encoding anti-TROP-2 antibody h7E6_SVGL variable light chain.

SEQ ID NO:331 is the nucleotide sequence encoding anti-TROP-2 antibody m6G11 variable heavy chain.

SEQ ID NO:332 is the nucleotide sequence encoding anti-TROP-2 antibody m6G11 variable light chain.

SEQ ID NO:333 is the nucleotide sequence encoding anti-TROP-2 antibody h6G11 variable heavy chain.

SEQ ID NO:334 is the nucleotide sequence encoding anti-TROP-2 antibody h6G11 variable light chain.

SEQ ID NO:335 is the nucleotide sequence encoding anti-TROP-2 antibody h6G11-FKG_SF variable heavy chain.

SEQ ID NO:336 is the nucleotide sequence encoding anti-TROP-2 antibody h6G11-FKG_SF variable light chain.

SEQ ID NO:337 is an anti-TROP-2 sacituzumab variable heavy chain amino acid sequence.

SEQ ID NO:338 is an anti-TROP-2 sacituzumab variable light chain amino acid sequence.

SEQ ID NO:339 is an anti-TROP-2 sacituzumab heavy chain CDR1 amino acid sequence.

SEQ ID NO:340 is an anti-TROP-2 sacituzumab heavy chain CDR2 amino acid sequence.

SEQ ID NO:341 is an anti-TROP-2 sacituzumab heavy chain CDR3 amino acid sequence.

SEQ ID NO:342 is an anti-TROP-2 sacituzumab light chain CDR1 amino acid sequence.

SEQ ID NO:343 is an anti-TROP-2 sacituzumab light chain CDR2 amino acid sequence.

SEQ ID NO:344 is an anti-TROP-2 sacituzumab light chain CDR3 amino acid sequence.

SEQ ID NO:345 is the amino acid sequence of anti-EPCAM scFv antibody 3-171 scFv.

SEQ ID NO:346 is the amino acid sequence of anti-EPCAM scFv antibody 7-F17 scFv.

SEQ ID NO:347 is the amino acid sequence of anti-EPCAM scFv antibody 12-C15 scFv.

SEQ ID NO:348 is the amino acid sequence of anti-EPCAM scFv antibody 16-G5 scFv.

SEQ ID NO:349 is the amino acid sequence of anti-EPCAM scFv antibody 17-C20 scFv.

SEQ ID NO:350 is the amino acid sequence of anti-EPCAM scFv antibody 24-G6 scFv.

SEQ ID NO:351 is an anti-EPCAM antibody variable heavy chain amino acid sequence.

SEQ ID NO:352 is an anti-EPCAM antibody variable light chain amino acid sequence.

SEQ ID NO:353 is an anti-EPCAM antibody variable light chain amino acid sequence.

SEQ ID NO:354 is an anti-EPCAM antibody variable light chain amino acid sequence.

SEQ ID NO:355 is an anti-EPCAM antibody variable light chain amino acid sequence.

SEQ ID NO:356 is an anti-EPCAM antibody variable light chain amino acid sequence.

SEQ ID NO:357 is an anti-EPCAM antibody variable light chain amino acid sequence.

SEQ ID NO:358 is an anti-EPCAM antibody heavy chain CDR1 amino acid sequence.

SEQ ID NO:359 is an anti-EPCAM antibody heavy chain CDR2 amino acid sequence.

SEQ ID NO:360 is an anti-EPCAM antibody heavy chain CDR3 amino acid sequence.

SEQ ID NO:361 is an anti-EPCAM antibody light chain CDR1 amino acid sequence.

SEQ ID NO:362 is an anti-EPCAM antibody light chain CDR2 amino acid sequence.

SEQ ID NO:363 is an anti-EPCAM antibody light chain CDR3 amino acid sequence.

SEQ ID NO:364 is the nucleotide sequence encoding anti-EPCAM scFv antibody 3-171 scFv.

SEQ ID NO:365 is the nucleotide sequence encoding anti-EPCAM scFv antibody 7-F17 scFv.

SEQ ID NO:366 is the nucleotide sequence encoding anti-EPCAM scFv antibody 12-C15 scFv.

SEQ ID NO:366 is the nucleotide sequence encoding anti-EPCAM scFv antibody 16-G5 scFv.

SEQ ID NO:367 is the nucleotide sequence encoding anti-EPCAM scFv antibody 17-C20 scFv.

SEQ ID NO:368 is the nucleotide sequence encoding anti-EPCAM scFv antibody 24-G6 scFv.

SEQ ID NO:369 is the nucleotide sequence encoding an anti-EPCAM scFv variable heavy chain.

SEQ ID NO:370 is the nucleotide sequence encoding an anti-EPCAM scFv variable light chain.

SEQ ID NO:371 is the nucleotide sequence encoding an anti-EPCAM scFv variable light chain.

SEQ ID NO:372 is the nucleotide sequence encoding an anti-EPCAM scFv variable light chain.

SEQ ID NO:373 is the nucleotide sequence encoding an anti-EPCAM scFv variable light chain.

SEQ ID NO:374 is the nucleotide sequence encoding an anti-EPCAM scFv variable light chain.

SEQ ID NO:375 is the nucleotide sequence encoding an anti-EPCAM scFv variable light chain.

SEQ ID NO:376 is the nucleotide sequence encoding an anti-EPCAM scFv variable light chain.

SEQ ID NO:377 is an anti-EPCAM antibody variable heavy chain amino acid sequence.

SEQ ID NO:378 is an anti-EPCAM antibody variable light chain amino acid sequence.

SEQ ID NO:379 is an anti-EPCAM antibody heavy chain CDR1 amino acid sequence.

SEQ ID NO:380 is an anti-EPCAM antibody heavy chain CDR2 amino acid sequence.

SEQ ID NO:381 is an anti-EPCAM antibody heavy chain CDR3 amino acid sequence.

SEQ ID NO:382 is an anti-EPCAM antibody light chain CDR1 amino acid sequence.

SEQ ID NO:383 is an anti-EPCAM antibody light chain CDR2 amino acid sequence.

SEQ ID NO:384 is an anti-EPCAM antibody light chain CDR3 amino acid sequence.

SEQ ID NO:385 is the amino acid sequence of anti-tissue factor antibody TF260 variable heavy chain.

SEQ ID NO:386 is the amino acid sequence of anti-tissue factor antibody TF260 variable light chain.

SEQ ID NO:387 is the amino acid sequence of anti-tissue factor antibody TF196 variable heavy chain.

SEQ ID NO:388 is the amino acid sequence of anti-tissue factor antibody TF196 variable light chain.

SEQ ID NO:389 is the amino acid sequence of anti-tissue factor antibody TF278 variable heavy chain.

SEQ ID NO:390 is the amino acid sequence of anti-tissue factor antibody TF278 variable light chain.

SEQ ID NO:391 is the amino acid sequence of anti-tissue factor antibody TF277 variable heavy chain.

SEQ ID NO:392 is the amino acid sequence of anti-tissue factor antibody TF277 variable light chain.

SEQ ID NO:393 is the amino acid sequence of anti-tissue factor antibody TF392 variable heavy chain.

SEQ ID NO:394 is the amino acid sequence of anti-tissue factor antibody TF392 variable light chain.

SEQ ID NO:395 is the amino acid sequence of anti-tissue factor antibody TF9 variable heavy chain.

SEQ ID NO:396 is the amino acid sequence of anti-tissue factor antibody TF9 variable light chain.

SEQ ID NO:397 is anti-tissue factor antibody TF260 heavy chain CDR1 amino acid sequence.

SEQ ID NO:398 is anti-tissue factor antibody TF260 heavy chain CDR2 amino acid sequence.

SEQ ID NO:399 is anti-tissue factor antibody TF260 heavy chain CDR3 amino acid sequence.

SEQ ID NO:400 is anti-tissue factor antibody TF260 light chain CDR1 amino acid sequence.

SEQ ID NO:401 is anti-tissue factor antibody TF260 light chain CDR2 amino acid sequence.

SEQ ID NO:402 is anti-tissue factor antibody TF260 light chain CDR3 amino acid sequence.

SEQ ID NO:403 is anti-tissue factor antibody TF196 heavy chain CDR1 amino acid sequence.

SEQ ID NO:404 is anti-tissue factor antibody TF196 heavy chain CDR2 amino acid sequence.

SEQ ID NO:405 is anti-tissue factor antibody TF196 heavy chain CDR3 amino acid sequence.

SEQ ID NO:406 is anti-tissue factor antibody TF196 light chain CDR1 amino acid sequence.

SEQ ID NO:407 is anti-tissue factor antibody TF196 light chain CDR2 amino acid sequence.

SEQ ID NO:408 is anti-tissue factor antibody TF196 light chain CDR3 amino acid sequence.

SEQ ID NO:409 is anti-tissue factor antibody TF9 heavy chain CDR1 amino acid sequence.

SEQ ID NO:410 is anti-tissue factor antibody TF9 heavy chain CDR2 amino acid sequence.

SEQ ID NO:411 is anti-tissue factor antibody TF9 heavy chain CDR3 amino acid sequence.

SEQ ID NO:412 is anti-tissue factor antibody TF9 light chain CDR1 amino acid sequence.

SEQ ID NO:413 is anti-tissue factor antibody TF9 light chain CDR2 amino acid sequence.

SEQ ID NO:414 is anti-tissue factor antibody TF9 light chain CDR3 amino acid sequence.

SEQ ID NO:415 is an amino acid sequence of an anti-tissue factor antibody variable heavy chain.

SEQ ID NO:416 is an amino acid sequence of an anti-tissue factor antibody variable light chain.

SEQ ID NO:417 is an amino acid sequence of an anti-tissue factor antibody variable heavy chain.

SEQ ID NO:418 is an amino acid sequence of an anti-tissue factor antibody variable light chain.

SEQ ID NO:419 is an amino acid sequence of an anti-tissue factor antibody variable heavy chain.

SEQ ID NO:420 is an amino acid sequence of an anti-tissue factor antibody variable light chain.

SEQ ID NO:421 is an amino acid sequence of an anti-tissue factor antibody variable heavy chain.

SEQ ID NO:422 is an amino acid sequence of an anti-tissue factor antibody variable light chain.

SEQ ID NO:423 is an amino acid sequence of an anti-tissue factor antibody variable heavy chain.

SEQ ID NO:424 is an amino acid sequence of an anti-tissue factor antibody variable light chain.

SEQ ID NO:425 is a nucleotide sequence encoding anti-tissue factor antibody TF260 variable heavy chain.

SEQ ID NO:426 is a nucleotide sequence encoding anti-tissue factor antibody TF260 variable light chain.

SEQ ID NO:427 is a nucleotide sequence encoding anti-tissue factor antibody TF196 variable heavy chain.

SEQ ID NO:428 is a nucleotide sequence encoding anti-tissue factor antibody TF196 variable light chain.

SEQ ID NO:429 is a nucleotide sequence encoding anti-tissue factor antibody TF278 variable heavy chain.

SEQ ID NO:430 is a nucleotide sequence encoding anti-tissue factor antibody TF278 variable light chain.

SEQ ID NO:431 is a nucleotide sequence encoding anti-tissue factor antibody TF277 variable heavy chain.

SEQ ID NO:432 is a nucleotide sequence encoding anti-tissue factor antibody TF277 variable light chain.

SEQ ID NO:433 is a nucleotide sequence encoding anti-tissue factor antibody TF392 variable heavy chain.

SEQ ID NO:434 is a nucleotide sequence encoding anti-tissue factor antibody TF392 variable light chain.

SEQ ID NO:435 is a nucleotide sequence encoding anti-tissue factor antibody TF9 variable heavy chain.

SEQ ID NO:436 is a nucleotide sequence encoding anti-tissue factor antibody TF9 variable light chain.

SEQ ID NO:437 is an amino acid sequence of an anti-LFA-1 or anti-CD11a antibody variable heavy chain.

SEQ ID NO:438 is an amino acid sequence of an anti-LFA-1 or anti-CD11a antibody variable light chain.

SEQ ID NO:439 is an amino acid sequence of an anti-LFA-1 or anti-CD11a antibody variable heavy chain.

SEQ ID NO:440 is an amino acid sequence of an anti-LFA-1 or anti-CD11a antibody variable light chain.

SEQ ID NO:441 is an anti-LFA-1 or anti-CD11a antibody heavy chain CDR1 amino acid sequence.

SEQ ID NO:442 is an anti-LFA-1 or anti-CD11a antibody heavy chain CDR2 amino acid sequence.

SEQ ID NO:443 is an anti-LFA-1 or anti-CD11a antibody heavy chain CDR3 amino acid sequence.

SEQ ID NO:444 is an anti-LFA-1 or anti-CD11a antibody light chain CDR1 amino acid sequence.

SEQ ID NO:445 is an anti-LFA-1 or anti-CD11a antibody light chain CDR2 amino acid sequence.

SEQ ID NO:446 is an anti-LFA-1 or anti-CD11a antibody light chain CDR3 amino acid sequence.

SEQ ID NO:447 is an amino acid sequence of an anti-FAP scFv based on sibrotuzumab.

SEQ ID NO:448 is the amino acid sequence of anti-FAP antibody sibrotuzumab variable heavy chain.

SEQ ID NO:449 is the amino acid sequence of anti-FAP antibody sibrotuzumab variable light chain.

SEQ ID NO:450 is the amino acid sequence of anti-FAP antibody FAP5 variable heavy chain.

SEQ ID NO:451 is the amino acid sequence of anti-FAP antibody FAP5 variable light chain.

SEQ ID NO:452 is a nucleotide sequence encoding anti-FAP antibody sibrotuzumab variable heavy chain.

SEQ ID NO:453 is a nucleotide sequence encoding anti-FAP antibody sibrotuzumab variable light chain.

SEQ ID NO:454 is an amino acid sequence of anti-VISTA antibody 1B8 variable heavy chain.

SEQ ID NO:455 is an amino acid sequence of anti-VISTA antibody 1B8 variable light chain.

SEQ ID NO:456 is an amino acid sequence of anti-VISTA antibody 1B8 heavy chain CDR1.

SEQ ID NO:457 is an amino acid sequence of anti-VISTA antibody 1B8 heavy chain CDR2.

SEQ ID NO:458 is an amino acid sequence of anti-VISTA antibody 1B8 heavy chain CDR3.

SEQ ID NO:459 is an amino acid sequence of anti-VISTA antibody 1B8 light chain CDR1.

SEQ ID NO:460 is an amino acid sequence of anti-VISTA antibody 1B8 light chain CDR2.

SEQ ID NO:461 is an amino acid sequence of anti-VISTA antibody 1B8 light chain CDR3.

SEQ ID NO:462 is an amino acid sequence of anti-VISTA antibody 2C12 variable heavy chain.

SEQ ID NO:463 is an amino acid sequence of anti-VISTA antibody 2C12 variable light chain.

SEQ ID NO:464 is an amino acid sequence of anti-VISTA antibody 2C12 heavy chain CDR1.

SEQ ID NO:465 is an amino acid sequence of anti-VISTA antibody 2C12 heavy chain CDR2.

SEQ ID NO:466 is an amino acid sequence of anti-VISTA antibody 2C12 heavy chain CDR3.

SEQ ID NO:467 is an amino acid sequence of anti-VISTA antibody 2C12 light chain CDR1.

SEQ ID NO:468 is an amino acid sequence of anti-VISTA antibody 2C12 light chain CDR2.

SEQ ID NO:469 is an amino acid sequence of anti-VISTA antibody 2C12 light chain CDR3.

SEQ ID NO:470 is an amino acid sequence of anti-VISTA antibody 1A12 variable heavy chain.

SEQ ID NO:471 is an amino acid sequence of anti-VISTA antibody 1A12 variable light chain.

SEQ ID NO:472 is an amino acid sequence of anti-VISTA antibody 1A12 heavy chain CDR1.

SEQ ID NO:473 is an amino acid sequence of anti-VISTA antibody 1A12 heavy chain CDR2.

SEQ ID NO:474 is an amino acid sequence of anti-VISTA antibody 1A12 heavy chain CDR3.

SEQ ID NO:475 is an amino acid sequence of anti-VISTA antibody 1A12 light chain CDR1.

SEQ ID NO:476 is an amino acid sequence of anti-VISTA antibody 1A12 light chain CDR2.

SEQ ID NO:477 is an amino acid sequence of anti-VISTA antibody 1A12 light chain CDR3.

SEQ ID NO:478 is an amino acid sequence of anti-VISTA antibody 3C5 variable heavy chain.

SEQ ID NO:479 is an amino acid sequence of anti-VISTA antibody 3C5 variable light chain.

SEQ ID NO:480 is an amino acid sequence of anti-VISTA antibody 3C5 heavy chain CDR1.

SEQ ID NO:481 is an amino acid sequence of anti-VISTA antibody 3C5 heavy chain CDR2.

SEQ ID NO:482 is an amino acid sequence of anti-VISTA antibody 3C5 heavy chain CDR3.

SEQ ID NO:483 is an amino acid sequence of anti-VISTA antibody 3C5 light chain CDR1.

SEQ ID NO:484 is an amino acid sequence of anti-VISTA antibody 3C5 light chain CDR2.

SEQ ID NO:485 is an amino acid sequence of anti-VISTA antibody 3C5 light chain CDR3.

SEQ ID NO:486 is an amino acid sequence of anti-LRRC15 antibody huM25 variable heavy chain.

SEQ ID NO:487 is an amino acid sequence of anti-LRRC15 antibody huM25 variable light chain.

SEQ ID NO:488 is an amino acid sequence of anti-LRRC15 antibody huM25 heavy chain CDR1.

SEQ ID NO:489 is an amino acid sequence of anti-LRRC15 antibody huM25 heavy chain CDR2.

SEQ ID NO:490 is an amino acid sequence of anti-LRRC15 antibody huM25 heavy chain CDR3.

SEQ ID NO:491 is an amino acid sequence of anti-LRRC15 antibody huM25 light chain CDR1.

SEQ ID NO:492 is an amino acid sequence of anti-LRRC15 antibody huM25 light chain CDR2.

SEQ ID NO:493 is an amino acid sequence of anti-LRRC15 antibody huM25 light chain CDR3.

SEQ ID NO:494 is an amino acid sequence of anti-LRRC15 antibody huAD208.4.1 variable heavy chain.

SEQ ID NO:495 is an amino acid sequence of anti-LRRC15 antibody huAD208.4.1 variable light chain.

SEQ ID NO:496 is an amino acid sequence of anti-LRRC15 antibody huAD208.4.1 heavy chain CDR1.

SEQ ID NO:497 is an amino acid sequence of anti-LRRC15 antibody huAD208.4.1 heavy chain CDR2.

SEQ ID NO:498 is an amino acid sequence of anti-LRRC15 antibody huAD208.4.1 heavy chain CDR3.

SEQ ID NO:499 is an amino acid sequence of anti-LRRC15 antibody huAD208.4.1 light chain CDR1.

SEQ ID NO:500 is an amino acid sequence of anti-LRRC15 antibody huAD208.4.1 light chain CDR2.

SEQ ID NO:501 is an amino acid sequence of anti-LRRC15 antibody huAD208.4.1 light chain CDR3.

SEQ ID NO:502 is an amino acid sequence of anti-LRRC15 antibody huAD208.12.1 variable heavy chain.

SEQ ID NO:503 is an amino acid sequence of anti-LRRC15 antibody huAD208.12.1 variable light chain.

SEQ ID NO:504 is an amino acid sequence of anti-LRRC15 antibody huAD208.12.1 heavy chain CDR1.

SEQ ID NO:505 is an amino acid sequence of anti-LRRC15 antibody huAD208.12.1 heavy chain CDR2.

SEQ ID NO:506 is an amino acid sequence of anti-LRRC15 antibody huAD208.12.1 heavy chain CDR3.

SEQ ID NO:507 is an amino acid sequence of anti-LRRC15 antibody huAD208.12.1 light chain CDR1.

SEQ ID NO:508 is an amino acid sequence of anti-LRRC15 antibody huAD208.12.1 light chain CDR2.

SEQ ID NO:509 is an amino acid sequence of anti-LRRC15 antibody huAD208.12.1 light chain CDR3.

SEQ ID NO:510 is an amino acid sequence of anti-LRRC15 antibody huAD208.14.1 variable heavy chain.

SEQ ID NO:511 is an amino acid sequence of anti-LRRC15 antibody huAD208.14.1 variable light chain.

SEQ ID NO:512 is an amino acid sequence of anti-LRRC15 antibody huAD208.14.1 heavy chain CDR1.

SEQ ID NO:513 is an amino acid sequence of anti-LRRC15 antibody huAD208.14.1 heavy chain CDR2.

SEQ ID NO:514 is an amino acid sequence of anti-LRRC15 antibody huAD208.14.1 heavy chain CDR3.

SEQ ID NO:515 is an amino acid sequence of anti-LRRC15 antibody huAD208.14.1 light chain CDR1.

SEQ ID NO:516 is an amino acid sequence of anti-LRRC15 antibody huAD208.14.1 light chain CDR2.

SEQ ID NO:517 is an amino acid sequence of anti-LRRC15 antibody huAD208.14.1 light chain CDR3.

SEQ ID NO:518 is an amino acid sequence of anti-LRRC15 antibody hu139.10 variable heavy chain.

SEQ ID NO:519 is an amino acid sequence of anti-LRRC15 antibody hu139.10 variable light chain.

SEQ ID NO:520 is an amino acid sequence of anti-LRRC15 antibody hu139.10 heavy chain CDR1.

SEQ ID NO:521 is an amino acid sequence of anti-LRRC15 antibody hu139.10 heavy chain CDR2.

SEQ ID NO:522 is an amino acid sequence of anti-LRRC15 antibody hu139.10 heavy chain CDR3.

SEQ ID NO:523 is an amino acid sequence of anti-LRRC15 antibody hu139.10 light chain CDR1.

SEQ ID NO:524 is an amino acid sequence of anti-LRRC15 antibody hu139.10 light chain CDR2.

SEQ ID NO:525 is an amino acid sequence of anti-LRRC15 antibody hu139.10 light chain CDR3.

SEQ ID NO:526 is an amino acid sequence of anti-LRRC15 antibody muAD210.40.9 variable heavy chain.

SEQ ID NO:527 is an amino acid sequence of anti-LRRC15 antibody muAD210.40.9 variable light chain.

SEQ ID NO:528 is an amino acid sequence of anti-LRRC15 antibody muAD210.40.9 heavy chain CDR1.

SEQ ID NO:529 is an amino acid sequence of anti-LRRC15 antibody muAD210.40.9 heavy chain CDR2.

SEQ ID NO:530 is an amino acid sequence of anti-LRRC15 antibody muAD210.40.9 heavy chain CDR3.

SEQ ID NO:531 is an amino acid sequence of anti-LRRC15 antibody muAD210.40.9 light chain CDR1.

SEQ ID NO:532 is an amino acid sequence of anti-LRRC15 antibody muAD210.40.9 light chain CDR2.

SEQ ID NO:533 is an amino acid sequence of anti-LRRC15 antibody muAD210.40.9 light chain CDR3.

SEQ ID NO:534 is an amino acid sequence of anti-LRRC15 antibody muAD209.9.1 variable heavy chain.

SEQ ID NO:535 is an amino acid sequence of anti-LRRC15 antibody muAD209.9.1 variable light chain.

SEQ ID NO:536 is an amino acid sequence of anti-LRRC15 antibody muAD209.9.1 heavy chain CDR1.

SEQ ID NO:537 is an amino acid sequence of anti-LRRC15 antibody muAD209.9.1 heavy chain CDR2.

SEQ ID NO:538 is an amino acid sequence of anti-LRRC15 antibody muAD209.9.1 heavy chain CDR3.

SEQ ID NO:539 is an amino acid sequence of anti-LRRC15 antibody muAD209.9.1 light chain CDR1.

SEQ ID NO:540 is an amino acid sequence of anti-LRRC15 antibody muAD209.9.1 light chain CDR2.

SEQ ID NO:541 is an amino acid sequence of anti-LRRC15 antibody muAD209.9.1 light chain CDR3.

SEQ ID NO:542 is an amino acid sequence of anti-B7-H3 antibody hBRCA84D variable heavy chain.

SEQ ID NO:543 is an amino acid sequence of anti-B7-H3 antibody hBRCA84D variable light chain.

SEQ ID NO:544 is an amino acid sequence of anti-B7-H3 antibody hBRCA84D heavy chain CDR1.

SEQ ID NO:545 is an amino acid sequence of anti-B7-H3 antibody hBRCA84D heavy chain CDR2.

SEQ ID NO:546 is an amino acid sequence of anti-B7-H3 antibody hBRCA84D heavy chain CDR3.

SEQ ID NO:547 is an amino acid sequence of anti-B7-H3 antibody hBRCA84D light chain CDR1.

SEQ ID NO:548 is an amino acid sequence of anti-B7-H3 antibody hBRCA84D light chain CDR2.

SEQ ID NO:549 is an amino acid sequence of anti-B7-H3 antibody hBRCA84D light chain CDR3.

SEQ ID NO:550 is an amino acid sequence of anti-B7-H3 antibody hBRCA84D variable heavy chain.

SEQ ID NO:551 is an amino acid sequence of anti-B7-H3 antibody hBRCA84D variable light chain.

SEQ ID NO:552 is an amino acid sequence of a PD-1 transmembrane domain.

SEQ ID NO:553 is an amino acid sequence of a CD28 transmembrane domain.

SEQ ID NO:554 is an amino acid sequence of a CD27 transmembrane domain.

SEQ ID NO:555 is an amino acid sequence of a CD8α transmembrane domain.

SEQ ID NO:556 is an amino acid sequence of a CD8α hinge domain.

SEQ ID NO:557 is an amino acid sequence of an IL-2Rβ hinge domain.

SEQ ID NO:558 is an amino acid sequence of an IgG1 transmembrane and hinge domain.

SEQ ID NO:559 is an amino acid sequence of an IgG1 hinge domain.

SEQ ID NO:560 is an amino acid sequence of an IgG4 hinge domain.

SEQ ID NO:561 is an amino acid sequence of an IgD hinge domain.

SEQ ID NO:562 is a nucleotide sequence encoding a PD-1 transmembrane domain.

SEQ ID NO:563 is a nucleotide sequence encoding a CD28 transmembrane domain.

SEQ ID NO:564 is a nucleotide sequence encoding a CD27 transmembrane domain.

SEQ ID NO:565 is a nucleotide sequence encoding a CD8α transmembrane domain.

SEQ ID NO:566 is a nucleotide sequence encoding a CD8α hinge domain.

SEQ ID NO:567 is a nucleotide sequence encoding an IL-2Rβ hinge domain.

SEQ ID NO: 568 is a nucleotide sequence encoding an IgG1 transmembrane and hinge domain.

SEQ ID NO:569 is a nucleotide sequence encoding an IgG1 hinge domain.

SEQ ID NO:570 is a nucleotide sequence encoding an IgG4 hinge domain.

SEQ ID NO:571 is a nucleotide sequence encoding an IgD hinge domain.

SEQ ID NO:572 is an amino acid sequence of a CD28 intracellular domain.

SEQ ID NO:573 is an amino acid sequence of a CD134 (OX40) intracellular domain.

SEQ ID NO:574 is an amino acid sequence of a CD278 (ICOS) intracellular domain.

SEQ ID NO:575 is an amino acid sequence of a CD137 (4-1BB) intracellular domain.

SEQ ID NO:576 is an amino acid sequence of a CD27 intracellular domain.

SEQ ID NO:577 is an amino acid sequence of a CD3ζ intracellular domain.

SEQ ID NO:578 is an amino acid sequence of an IL-2Rβ intracellular domain.

SEQ ID NO:579 is an amino acid sequence of an IL-2Rγ intracellular domain.

SEQ ID NO:580 is an amino acid sequence of an IL-18R1 intracellular domain.

SEQ ID NO:581 is an amino acid sequence of an IL-7Rα intracellular domain.

SEQ ID NO:582 is an amino acid sequence of an IL-12R1 intracellular domain.

SEQ ID NO: 583 is an amino acid sequence of an IL-12R2 intracellular domain.

SEQ ID NO:584 is an amino acid sequence of an IL-15Rα intracellular domain.

SEQ ID NO:585 is an amino acid sequence of an IL-21R intracellular domain.

SEQ ID NO: 586 is an amino acid sequence of a LTBR intracellular domain.

SEQ ID NO: 587 is an amino acid sequence of a linker.

SEQ ID NO:588 is a nucleotide sequence encoding a CD28 intracellular domain.

SEQ ID NO:589 is a nucleotide sequence encoding a CD134 (OX40) intracellular domain.

SEQ ID NO:590 is a nucleotide sequence encoding a CD278 (ICOS) intracellular domain.

SEQ ID NO:591 is a nucleotide sequence encoding a CD137 (4-1BB) intracellular domain.

SEQ ID NO:592 is a nucleotide sequence encoding a CD27 intracellular domain.

SEQ ID NO: 593 is a nucleotide sequence encoding a CD3ζ intracellular domain.

SEQ ID NO: 594 is a nucleotide sequence encoding an IL-2Rβ intracellular domain.

SEQ ID NO:595 is a nucleotide sequence encoding an IL-2Rγ intracellular domain.

SEQ ID NO:596 is a nucleotide sequence encoding an IL-18R1 intracellular domain.

SEQ ID NO:597 is a nucleotide sequence encoding an IL-7Rα intracellular domain.

SEQ ID NO:598 is a nucleotide sequence encoding an IL-12R1 intracellular domain.

SEQ ID NO:599 is a nucleotide sequence encoding an IL-12R2 intracellular domain.

SEQ ID NO:600 is a nucleotide sequence encoding an IL-15Rα intracellular domain.

SEQ ID NO:601 is a nucleotide sequence encoding an IL-21R intracellular domain.

SEQ ID NO:602 is a nucleotide sequence encoding a LTBR intracellular domain.

SEQ ID NO:603 is a nucleotide sequence encoding a linker.

SEQ ID NO:604 is a nucleotide sequence for an EF-1 promoter.

SEQ ID NO:605 is a nucleotide sequence for a CMV promoter.

SEQ ID NO:606 is a nucleotide sequence for an MSCV promoter.

SEQ ID NO:607 is a nucleotide sequence for an NFAT promoter.

SEQ ID NO:608 is an amino acid sequence for a T2A self-cleaving peptide (derived from thosea asigna virus 2A).

SEQ ID NO:609 is an amino acid sequence for a P2A self-cleaving peptide (derived from porcine teschovirus-1 2A).

SEQ ID NO:610 is an amino acid sequence for a E2A self-cleaving peptide (derived from equine rhinitis A virus).

SEQ ID NO:611 is an amino acid sequence for a F2A self-cleaving peptide (derived from foot-and-mouth disease virus).

SEQ ID NO:612 is an amino acid sequence for a linker.

SEQ ID NO:613 is a nucleotide sequence encoding a T2A self-cleaving peptide.

SEQ ID NO:614 is a nucleotide sequence encoding a P2A self-cleaving peptide.

SEQ ID NO:615 is a nucleotide sequence encoding an E2A self-cleaving peptide.

SEQ ID NO:616 is a nucleotide sequence encoding a F2A self-cleaving peptide.

SEQ ID NO:617 is a nucleotide sequence encoding an IRES domain.

SEQ ID NO:618 is a nucleotide sequence for a vector encoding a CCR comprising (anti-TROP2-V_{L})-(linker)-(anti-TROP2-V_{H})-(IgG4 hinge and transmembrane)-(IL-2Rβ).

SEQ ID NO:619 is a nucleotide sequence for a vector encoding a CCR comprising (anti-FAP-V_{L})-(linker)-(anti-FAP-V_{H})-(CD8α hinge and transmembrane)-(IL-18R1).

SEQ ID NO:620 is a nucleotide sequence for a vector encoding a CCR comprising (anti-PD-L1-V_{L})-(linker)-(anti-PD-L1-V_{H})-(CD8α hinge and transmembrane)-(CD27), using the 38A1 anti-PD-L1 domains described herein.

SEQ ID NO:621 is a nucleotide sequence for a vector encoding two CCRs comprising SP-(38A1 scFv)-(CD28 hinge and transmembrane)-(IL-2Rβ intracellular)-T2A-SP-(19H9 scFv)-(CD28 hinge and transmembrane)-(IL-2Rγ intracellular), using both the 38A1 and 19H9 PD-L1 domains described herein. SP refers to a signal peptide.

SEQ ID NO:622 is a nucleotide sequence for a vector encoding two CCRs comprising SP-(38A1 scFv)-(CD28 hinge and transmembrane)-(IL-18R1 intracellular)-T2A-SP-(19H9 scFv)-(CD28 hinge and transmembrane)-(IL-18RAP intracellular), using both the 38A1 and 19H9 PD-L1 domains described herein. SP refers to a signal peptide.

SEQ ID NO:623 is a nucleotide sequence for a vector encoding two CCRs comprising SP-(anti-TROP-2 scFv)-(CD8 hinge)-(IL-2Rβ transmembrane and intracellular)-T2A-SP-(anti-TROP-2 scFv)-(CD8 hinge)-(IL-2Rγ transmembrane and intracellular). SP refers to a signal peptide.

SEQ ID NO:624 is a nucleotide sequence for a vector encoding two CCRs comprising SP-(anti-TROP-2 scFv)-(CD8 hinge)-(IL-18R1-transmembrane and intracellular)-T2A-SP-(anti-TROP-2 scFv)-(CD8 hinge)-(IL-18RAP-transmembrane and intracellular). SP refers to a signal peptide.

SEQ ID NO:625 is a nucleotide sequence for a vector encoding two CCRs comprising SP-(cAR47A6.4 scFv)-(CD28 hinge-transmembrane)-(IL-2Rβ intracellular)-T2A-SP-(KM4097 scFv)-(CD28 hinge and transmembrane)-(IL-2Rγ intracellular). SP refers to a signal peptide.

SEQ ID NO:626 is a nucleotide sequence for a vector encoding two CCRs comprising SP-(cAR47A6.4 scFv)-(CD28 hinge-transmembrane)-(IL-18R1 intracellular)-T2A-SP-(KM4097scFv)-(CD28 hinge-transmembrane)-(IL-18RAP intracellular). SP refers to a signal peptide.

SEQ ID NO:627 is an amino acid sequence of a CXCR1 domain.

SEQ ID NO:628 is an amino acid sequence of a CXCR2 variant 1 and 2 domain.

SEQ ID NO:629 is an amino acid sequence of a CXCR3 variant 1 domain.

SEQ ID NO:630 is an amino acid sequence of a CXCR3 variant 2 domain.

SEQ ID NO:631 is an amino acid sequence of a CXCR4 variant 1 domain.

SEQ ID NO:632 is an amino acid sequence of a CXCR4 variant 2 domain.

SEQ ID NO:633 is an amino acid sequence of a CXCR4 variant 3 domain.

SEQ ID NO:634 is an amino acid sequence of a CXCR4 variant 4 domain.

SEQ ID NO:635 is an amino acid sequence of a CXCR4 variant 5 domain.

SEQ ID NO:636 is an amino acid sequence of a CXCR5 variant 1 domain.

SEQ ID NO:637 is an amino acid sequence of a CXCR5 variant 2 domain.

SEQ ID NO:638 is an amino acid sequence of a CCR2 variant A domain.

SEQ ID NO:639 is an amino acid sequence of a CCR2 variant B domain.

SEQ ID NO:640 is an amino acid sequence of a CCR4 domain.

SEQ ID NO:641 is an amino acid sequence of a CCR6 variant 1 and 2 domain.

SEQ ID NO:642 is an amino acid sequence of a CCR7 variant 1 domain.

SEQ ID NO:643 is an amino acid sequence of a CCR7 variant 2 domain.

SEQ ID NO:644 is an amino acid sequence of a CCR7 variant 3, 4, and 5 domain.

SEQ ID NO:645 is an amino acid sequence of a CCR8 domain.

SEQ ID NO:646 is a nucleotide sequence encoding a CXCR1 domain.

SEQ ID NO:647 is a nucleotide sequence encoding a CXCR2 variant 1 domain.

SEQ ID NO:648 is a nucleotide sequence encoding a CXCR2 variant 2 domain.

SEQ ID NO:649 is a nucleotide sequence encoding a CXCR3 variant 1 domain.

SEQ ID NO:650 is a nucleotide sequence encoding a CXCR3 variant 2 domain.

SEQ ID NO:651 is a nucleotide sequence encoding a CXCR4 variant 1 domain.

SEQ ID NO:652 is a nucleotide sequence encoding a CXCR4 variant 2 domain.

SEQ ID NO:653 is a nucleotide sequence encoding a CXCR4 variant 3 domain.

SEQ ID NO:654 is a nucleotide sequence encoding a CXCR4 variant 4 domain.

SEQ ID NO:655 is a nucleotide sequence encoding a CXCR4 variant 5 domain.

SEQ ID NO:656 is a nucleotide sequence encoding a CXCR5 variant 1 domain.

SEQ ID NO:657 is a nucleotide sequence encoding a CXCR5 variant 2 domain.

SEQ ID NO:658 is a nucleotide sequence encoding a CCR2 variant A domain.

SEQ ID NO:659 is a nucleotide sequence encoding a CCR2 variant B domain.

SEQ ID NO:660 is a nucleotide sequence encoding a CCR4 domain.

SEQ ID NO:661 is a nucleotide sequence encoding a CCR6 variant 1 domain.

SEQ ID NO:662 is a nucleotide sequence encoding a CCR6 variant 2 domain.

SEQ ID NO:663 is a nucleotide sequence encoding a CCR7 variant 1 domain.

SEQ ID NO:664 is a nucleotide sequence encoding a CCR7 variant 2 domain.

SEQ ID NO:665 is a nucleotide sequence encoding a CCR7 variant 3 domain.

SEQ ID NO:666 is a nucleotide sequence encoding a CCR7 variant 4 domain.

SEQ ID NO:667 is a nucleotide sequence encoding a CCR7 variant 5 domain.

SEQ ID NO:668 is a nucleotide sequence encoding a CCR8 domain.

SEQ ID NO:669 is a nucleotide sequence for a vector encoding a CXCR1 chemokine receptor.

SEQ ID NO:670 is a nucleotide sequence for a vector encoding a CCR8 chemokine receptor.

SEQ ID NO:671 is an amino acid sequence for two CCRs comprising SP-(38A1 scFv)-(CD28 hinge and transmembrane)-(IL-2Rβ intracellular)-T2A-SP-(19H9 scFv)-(CD28 hinge and transmembrane)-(IL-2Rγ intracellular), using both the 38A1 and 19H9 PD-L1 domains described herein. SP refers to a signal peptide.

SEQ ID NO:672 is an amino acid sequence for two CCRs comprising SP-(38A1 scFv)-(CD28 hinge and transmembrane)-(IL-18R1 intracellular)-T2A-SP-(19H9 scFv)-(CD28 hinge and transmembrane)-(IL-18RAP intracellular), using both the 38A1 and 19H9 PD-L1 domains described herein. SP refers to a signal peptide.

SEQ ID NO:673 is an amino acid sequence for two CCRs comprising SP-(anti-TROP-2 scFv)-(CD8 hinge)-(IL-2Rβ transmembrane and intracellular)-T2A-SP-(anti-TROP-2 scFv)-(CD8 hinge)-(IL-2Rγ transmembrane and intracellular). SP refers to a signal peptide.

SEQ ID NO:674 is an amino acid sequence for two CCRs comprising SP-(anti-TROP-2 scFv)-(CD8 hinge)-(IL-18R1-transmembrane and intracellular)-T2A-SP-(anti-TROP-2 scFv)-(CD8 hinge)-(IL-18RAP-transmembrane and intracellular). SP refers to a signal peptide.

SEQ ID NO:675 is an amino acid sequence for two CCRs comprising SP-(cAR47A6.4 scFv)-(CD28 hinge-transmembrane)-(IL-2Rβ intracellular)-T2A-SP-(KM4097 scFv)-(CD28 hinge and transmembrane)-(IL-2Rγ intracellular). SP refers to a signal peptide.

SEQ ID NO:676 is an amino acid sequence for two CCRs comprising SP-(cAR47A6.4 scFv)-(CD28 hinge-transmembrane)-(IL-18R1 intracellular)-T2A-SP-(KM4097scFv)-(CD28 hinge-transmembrane)-(IL-18RAP intracellular). SP refers to a signal peptide.

SEQ ID NO:677 is an amino acid sequence for two CCRs comprising CCR7.2:chPD-L1-IL-2R (SP-38A1scFv-IL2Rβ12aaEC-TM-IL-2Rβ-IC-T2A-SP-19H9scFv-IL2Rγ12aaEC-TM-IL-2Rγ-IC). SP refers to a signal peptide, EC refers to extracellular, TM refers to transmembrane, and IC refers to intracellular.

SEQ ID NO:678 is an amino acid sequence for two CCRs comprising CCR8.2:chPD-L1-IL-18R (SP-38A1scFv-IL-18R112aaEC-TM-IL-18R1-IC-T2A-SP-19H9scFv-IL-18RRAP12aaEC-TM-IL-18RAP-IC). SP refers to a signal peptide, EC refers to extracellular, TM refers to transmembrane, and IC refers to intracellular.

SEQ ID NO:679 is an amino acid sequence for two CCRs comprising CCR11.2:TROP2-TL-2R (SP-cAR47A6.4 scFv-IL2Rβ12aaEC-TM-IL-2Rβ-TC-T2A-SP-KM4097scFV-IL2Rγ12aaEC-TM-IL-2Rγ-IC). SP refers to a signal peptide, EC refers to extracellular, TM refers to transmembrane, and IC refers to intracellular.

SEQ ID NO:680 is an amino acid sequence for two CCRs comprising CCR12.2:TROP2-IL-18R (SP-cAR47A6.4 scFv-IL-18R112aaEC-TM-IL-18R1-IC-T2A-SP-KM4097scFv-IL-18RRAP12aaEC-TM-IL-18RAP-IC). SP refers to a signal peptide, EC refers to extracellular, TM refers to transmembrane, and IC refers to intracellular.

SEQ ID NO:681 is a nucleotide sequence encoding CCR7.2.

SEQ ID NO:682 is a nucleotide sequence encoding CCR8.2.

SEQ ID NO:683 is a nucleotide sequence encoding CCR11.2.

SEQ ID NO:684 is a nucleotide sequence encoding CCR12.2.

SEQ ID NO:685 is a nucleotide sequence for a vector encoding CCR7.2.

SEQ ID NO:686 is a nucleotide sequence for a vector encoding CCR8.2.

SEQ ID NO:687 is a nucleotide sequence for a vector encoding CCR11.2.

SEQ ID NO:688 is a nucleotide sequence for a vector encoding CCR12.2.

SEQ ID NO:689 is an amino acid sequence for CCR13 (ch Fas-4-1BB).

SEQ ID NO:690 is an amino acid sequence for CCR14 (ch PD-1-4-1BB).

SEQ ID NO:691 is an amino acid sequence for CCR15 (TGFβRII-4-1BB).

SEQ ID NO:692 is an amino acid sequence for CCR16 (ch PD-1-CD28).

SEQ ID NO:693 is an amino acid sequence for a FAS binding domain.

SEQ ID NO:694 is an amino acid sequence for a TGFβRII binding domain.

SEQ ID NO:695 is a nucleotide sequence encoding CCR13 (ch Fas-4-1BB).

SEQ ID NO:696 is a nucleotide sequence encoding CCR14 (ch PD-1-4-1BB).

SEQ ID NO:697 is a nucleotide sequence encoding CCR15 (TGFβRII-4-1BB).

SEQ ID NO:698 is a nucleotide sequence encoding CCR16 (ch PD-1-CD28).

SEQ ID NO:699 is a nucleotide sequence for a vector encoding CCR13 (ch Fas-4-1BB).

SEQ ID NO:700 is a nucleotide sequence for a vector encoding CCR14 (ch PD-1-4-1BB).

SEQ ID NO:701 is a nucleotide sequence for a vector encoding CCR15 (ch TGFβRII-4-1BB).

SEQ ID NO:702 is a nucleotide sequence for a vector encoding CCR16 (ch PD-1-CD28).

SEQ ID NO:703 is an amino acid sequence for CCR17 (ch Fas-LTBR).

SEQ ID NO:704 is an amino acid sequence for CCR18 (ch PD-1-LTBR).

SEQ ID NO:705 is an amino acid sequence for CCR19 (ch TGFβRII-LTBR).

SEQ ID NO:706 is a nucleotide sequence encoding CCR17 (ch Fas-LTBR).

SEQ ID NO:707 is a nucleotide sequence encoding CCR18 (ch PD-1-LTBR).

SEQ ID NO:708 is a nucleotide sequence encoding CCR19 (ch TGFβRII-LTBR).

SEQ ID NO:709 is a nucleotide sequence for a vector encoding CCR17 (ch Fas-LTBR).

SEQ ID NO:710 is a nucleotide sequence for a vector encoding CCR18 (ch PD-1-LTBR).

SEQ ID NO:711 is a nucleotide sequence for a vector encoding CCR19 (ch TGFβRII-LTBR).

SEQ ID NO:712 is an amino acid sequence for CCR20 (ch 19H9-4-1BB).

SEQ ID NO:713 is an amino acid sequence for CCR21 (ch 19H9-LTBR).

SEQ ID NO:714 is an amino acid sequence for CCR22 (ch 19H9-4-1BB version 2).

SEQ ID NO:715 is an amino acid sequence for CCR23 (ch 19H9-LTBR version 2).

SEQ ID NO:716 is an amino acid sequence for CCR24 (ch 19H9-LTBR-4-1BB).

SEQ ID NO:717 is an amino acid sequence for CCR25 (ch 19H9-4-1BB-LTBR).

SEQ ID NO:718 is a nucleotide sequence encoding CCR20 (ch 19H9-4-1BB).

SEQ ID NO:719 is a nucleotide sequence encoding CCR21 (ch 19H9-LTBR).

SEQ ID NO:720 is a nucleotide sequence encoding CCR22 (ch 19H9-4-1BB version 2).

SEQ ID NO:721 is a nucleotide sequence encoding CCR23 (ch 19H9-LTBR version 2).

SEQ ID NO:722 is a nucleotide sequence encoding CCR24 (ch 19H9-LTBR-4-1BB).

SEQ ID NO:723 is a nucleotide sequence encoding CCR25 (ch 19H9-4-1BB-LTBR).

SEQ ID NOs: 724-815 are exemplary amino acid sequence for DLL3 antibody variable light chain region (VL1-VL92) comprising FR1, CDR1, FR2, CDR2, FR3, and CDR3.

SEQ ID NOs: 816-820 are exemplary amino acid sequence for humanized DLL3 antibody variable light chain region (VL93-VL97) comprising FR1, CDR1, FR2, CDR2, FR3, and CDR3.

SEQ ID NOs: 821-912 are exemplary amino acid sequence for DLL3 antibody variable heavy chain region (VH1-VH92) comprising FR1, CDR1, FR2, CDR2, FR3, and CDR3.

SEQ ID NOs: 913-917 are exemplary amino acid sequence for humanized DLL3 antibody variable heavy chain region (VH93-VH97) comprising FR1, CDR1, FR2, CDR2, FR3, and CDR3.

SEQ ID NO: 918 is an exemplary amino acid sequence for DLL3 antibody variable heavy chain region.

SEQ ID NO: 919 is an exemplary amino acid sequence for DLL3 antibody variable light chain region.

### DETAILED DESCRIPTION OF THE INVENTION

### I. Introduction

Adoptive cell therapy utilizing TILs cultured ex vivo by the rapid expansion protocol (REP) has produced successful adoptive cell therapy following host immunosuppression in patients with cancer such as melanoma. Current TIL manufacturing and treatment processes are limited by length, cost, sterility concerns, and other factors described herein. There is an urgent need to provide TIL manufacturing processes and therapies based on such processes that are appropriate for use in treating patients for whom very few or no viable treatment options remain. The present invention meets this need by providing a manufacturing process and product for use in generating TILs that have been modified using CCRs or chemokine receptors, amongst other modifications described herein, to improve their efficacy, potency, safety, sternness, or other measures of performance.

### II. Definitions

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering.” “administered in combination with,” “administering in combination with,” “simultaneous,” and “concurrent,” as used herein, encompass administration of two or more active pharmaceutical ingredients (in a preferred embodiment of the present invention, for example, a plurality of TILs) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs and expanded TILs (“REP TILs” or “post-REP TILs”). TIL cell populations can include genetically modified TILs.

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion.

By “cryopreserved TILs” herein is meant that TILs, either primary, bulk, or expanded (REP TILs), are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” herein is meant a population of TILs that was previously cryopreserved and then treated to return to room temperature or higher, including but not limited to cell culture temperatures or temperatures wherein TILs may be administered to a patient.

TILs can generally be defined either biochemically, using cell surface markers, or functionally, by their ability to infiltrate tumors and effect treatment. TILs can be generally categorized by expressing one or more of the following biomarkers: CD4, CD8, T-cell receptor (TCR) αβ, CD27, CD28, CD56, CCR7, CD45Ra, CD95, PD-1, and CD25. Additionally and alternatively, TILs can be functionally defined by their ability to infiltrate solid tumors upon reintroduction into a patient.

The term “cryopreservation media” or “cryopreservation medium” refers to any medium that can be used for cryopreservation of cells. Such media can include media comprising 7% to 10% DMSO. Exemplary media include CryoStor CS10, Hyperthermasol, as well as combinations thereof. The term “CS10” refers to a cryopreservation medium which is obtained from Stemcell Technologies or from Biolife Solutions. The CS10 medium may be referred to by the trade name “CryoStor® CS10”. The CS10 medium is a serum-free, animal component-free medium which comprises DMSO.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR7^{lo}) and are heterogeneous or low for CD62L expression (CD62L^{lo}). The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perform.

The term “closed system” refers to a system that is closed to the outside environment. Any closed system appropriate for cell culture methods can be employed with the methods of the present invention. Closed systems include, for example, but are not limited to closed G-containers. Once a tumor segment is added to the closed system, the system is no opened to the outside environment until the TILs are ready to be administered to the patient.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. When used as an antigen presenting cell (PBMCs are a type of antigen-presenting cell), the peripheral blood mononuclear cells are preferably irradiated allogeneic peripheral blood mononuclear cells.

The terms “peripheral blood lymphocytes” and “PBLs” refer to T cells expanded from peripheral blood. In some embodiments, PBLs are separated from whole blood or apheresis product from a donor. In some embodiments, PBLs are separated from whole blood or apheresis product from a donor by positive or negative selection of a T cell phenotype, such as the T cell phenotype of CD3+ CD45+.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD3E. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, CA, USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab (exemplary OKT-3 antibody).

| Identifier | Sequence (One-Letter Amino Acid Symbols) | |
|---|---|---|
| SEQ ID NO: 1 | QVQLQQSGAE LARPGASVKM SCKASGYTFT RYTMHWVKQR PGQGLEWIGY INPSRGYTNY | 60 |
| muromonab heavy | NQKFKDKATL TTDKSSSTAY MQLSSITSED SAVYYCARYY DDHYCLDYWG QGTTLTVSSA | 120 |
| chain | KTTAPSVYPL APVCGGTTGS SVTLGCLVKG YFPEPVTLTW NSGSLSSGVH TFPAVLQSDL | 180 |
| | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTIPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| muromonab light | FRGSGSGTSY SLTISGMEAE DAATYYCQQW SSNPFTFGSG TKLEINRADT APTVSIFPPS | 120 |
| chain | SEQLTSGGAS VVCFLNNFYP KDINVKWKID GSERQNGVLN SWTDQDSKDS TYSMSSTLTL | 180 |
| | TKDEYERHNS YTCEATHKTS TSPIVKSFNR NEC | 213 |

The term “IL-2” (also referred to herein as “IL2”) refers to the T cell growth factor known as interleukin-2 and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug bempegaldesleukin (NKTR-214, pegylated human recombinant IL-2 as in SEQ ID NO:4 in which an average of 6 lysine residues are N⁶ substituted with [(2,7-bis{[methylpoly(oxyethylene)]carbamoyl}-9H-fluoren-9-yl)methoxy]carbonyl), which is available from Nektar Therapeutics, South San Francisco, CA, USA, or which may be prepared by methods known in the art, such as the methods described in Example 19 of International Patent Application Publication No. WO 2018/132496 A1 or the method described in Example 1 of U.S. Patent Application Publication No. US 2019/0275133 A1, the disclosures of which are incorporated by reference herein. Bempegaldesleukin (NKTR-214) and other pegylated IL-2 molecules suitable for use in the invention are described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

In some embodiments, an IL-2 form suitable for use in the present invention is THOR-707, available from Synthorx, Inc. The preparation and properties of THOR-707 and additional alternative forms of IL-2 suitable for use in the invention are described in U.S. Patent Application Publication Nos. US 2020/0181220 A1 and US 2020/0330601 A1, the disclosures of which are incorporated by reference herein. In some embodiments, and IL-2 form suitable for use in the invention is an interleukin 2 (IL-2) conjugate comprising: an isolated and purified IL-2 polypeptide; and a conjugating moiety that binds to the isolated and purified IL-2 polypeptide at an amino acid position selected from K35, T37, R38, T41, F42, K43, F44, Y45, E61, E62, E68, K64, P65, V69, L72, and Y107, wherein the numbering of the amino acid residues corresponds to SEQ ID NO:5. In some embodiments, the amino acid position is selected from T37, R38, T41, F42, F44, Y45, E61, E62, E68, K64, P65, V69, L72, and Y107. In some embodiments, the amino acid position is selected from T37, R38, T41, F42, F44, Y45, E61, E62, E68, P65, V69, L72, and Y107. In some embodiments, the amino acid position is selected from T37, T41, F42, F44, Y45, P65, V69, L72, and Y107. In some embodiments, the amino acid position is selected from R38 and K64. In some embodiments, the amino acid position is selected from E61, E62, and E68. In some embodiments, the amino acid position is at E62. In some embodiments, the amino acid residue selected from K35, T37, R38, T41, F42, K43, F44, Y45, E61, E62, E68, K64, P65, V69, L72, and Y107 is further mutated to lysine, cysteine, or histidine. In some embodiments, the amino acid residue is mutated to cysteine. In some embodiments, the amino acid residue is mutated to lysine. In some embodiments, the amino acid residue selected from K35, T37, R38, T41, F42, K43, F44, Y45, E61, E62, E68, K64, P65, V69, L72, and Y107 is further mutated to an unnatural amino acid. In some embodiments, the unnatural amino acid comprises N6-azidoethoxy-L-lysine (AzK), N6-propargylethoxy-L-lysine (PraK), BCN-L-lysine, norbomene lysine, TCO-lysine, methyltetrazine lysine, allyloxycarbonyllysine, 2-amino-8-oxononanoic acid, 2-amino-8-oxooctanoic acid, p-acetyl-L-phenvlalanine, p-azidomethyl-L-phenvlalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, or selenocysteine. In some embodiments, the IL-2 conjugate has a decreased affinity to IL-2 receptor α (IL-2Rα) subunit relative to a wild-type IL-2 polypeptide. In some embodiments, the decreased affinity is about 10%, 20%, 30%, 40%, 50%, 60%, 70%, 80%, 90%, 95%, 99%, or greater than 99% decrease in binding affinity to IL-2Rα relative to a wild-type IL-2 polypeptide. In some embodiments, the decreased affinity is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 500-fold, 1000-fold, or more relative to a wild-type IL-2 polypeptide. In some embodiments, the conjugating moiety impairs or blocks the binding of IL-2 with IL-2Rα. In some embodiments, the conjugating moiety comprises a water-soluble polymer. In some embodiments, the additional conjugating moiety comprises a water-soluble polymer. In some embodiments, each of the water-soluble polymers independently comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), or a combination thereof. In some embodiments, each of the water-soluble polymers independently comprises PEG. In some embodiments, the PEG is a linear PEG or a branched PEG. In some embodiments, each of the water-soluble polymers independently comprises a polysaccharide. In some embodiments, the polysaccharide comprises dextran, polysialic acid (PSA), hyaluronic acid (HA), amylose, heparin, heparan sulfate (HS), dextrin, or hydroxyethyl-starch (HES). In some embodiments, each of the water-soluble polymers independently comprises a glycan. In some embodiments, each of the water-soluble polymers independently comprises polyamine. In some embodiments, the conjugating moiety comprises a protein. In some embodiments, the additional conjugating moiety comprises a protein. In some embodiments, each of the proteins independently comprises an albumin, a transferrin, or a transthyretin. In some embodiments, each of the proteins independently comprises an Fc portion. In some embodiments, each of the proteins independently comprises an Fe portion of IgG. In some embodiments, the conjugating moiety comprises a polypeptide. In some embodiments, the additional conjugating moiety comprises a polypeptide. In some embodiments, each of the polypeptides independently comprises a XTEN peptide, a glycine-rich homoamino acid polymer (HAP), a PAS polypeptide, an elastin-like polypeptide (ELP), a CTP peptide, or a gelatin-like protein (GLK) polymer. In some embodiments, the isolated and purified IL-2 polypeptide is modified by glutamylation. In some embodiments, the conjugating moiety is directly bound to the isolated and purified IL-2 polypeptide. In some embodiments, the conjugating moiety is indirectly bound to the isolated and purified IL-2 polypeptide through a linker. In some embodiments, the linker comprises a homobifunctional linker. In some embodiments, the homobifunctional linker comprises Lomant's reagent dithiobis (succinimidylpropionate) DSP, 3′3′-dithiobis(sulfosuccinimidyl proprionate) (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-(3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide). In some embodiments, the linker comprises a heterobifunctional linker. In some embodiments, the heterobifunctional linker comprises N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy) sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-(((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenyl amino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(p-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), 1-(p-azidosalicylamido)-4-(iodoacetamido)butane (AsIB), N-[4-(p-azidosalicylamido)butyl]-3′-(2′-pyridyldithio) propionamide (APDP), benzophenone-4-iodoacetamide, p-azidobenzoyl hydrazide (ABH), 4-(p-azidosalicylamido)butylamine (AsBA), or p-azidophenyl glyoxal (APG). In some embodiments, the linker comprises a cleavable linker, optionally comprising a dipeptide linker. In some embodiments, the dipeptide linker comprises Val-Cit, Phe-Lys, Val-Ala, or Val-Lys. In some embodiments, the linker comprises a non-cleavable linker. In some embodiments, the linker comprises a maleimide group, optionally comprising maleimidocaproyl (mc), succinimidyl-4-(N-maleimidomethyl)cyclohexane-1-carboxylate (sMCC), or sulfosuccinimidyl-4-(N-maleimidomethyl)cyclohexane-1-carboxylate (sulfo-sMCC). In some embodiments, the linker further comprises a spacer. In some embodiments, the spacer comprises p-aminobenzyl alcohol (PAB), p-aminobenzyoxycarbonyl (PABC), a derivative, or an analog thereof. In some embodiments, the conjugating moiety is capable of extending the serum half-life of the IL-2 conjugate. In some embodiments, the additional conjugating moiety is capable of extending the serum half-life of the IL-2 conjugate. In some embodiments, the IL-2 form suitable for use in the invention is a fragment of any of the IL-2 forms described herein. In some embodiments, the IL-2 form suitable for use in the invention is pegylated as disclosed in U.S. Patent Application Publication No. US 2020/0181220 A1 and U.S. Patent Application Publication No. US 2020/0330601 At. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 80% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 polypeptide comprises an N-terminal deletion of one residue relative to SEQ ID NO:5. In some embodiments, the IL-2 form suitable for use in the invention lacks IL-2R alpha chain engagement but retains normal binding to the intermediate affinity IL-2R beta-gamma signaling complex. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 90% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 95% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 98% sequence identity to SEQ ID NO:5: and the AzK substitutes for an amino acid at position K35, F42, F44, K43, E62, P65, R38, T41, E68, Y45, V69, or L72 in reference to the amino acid positions within SEQ ID NO:5.

In some embodiments, an IL-2 form suitable for use in the invention is nemvaleukin alfa, also known as ALKS-4230 (SEQ ID NO:6), which is available from Alkermes, Inc. Nemvaleukin alfa is also known as human interleukin 2 fragment (1-59), variant (Cys¹²⁵>Ser⁵¹), fused via peptidyl linker (⁶⁰GG⁶¹) to human interleukin 2 fragment (62-132), fused via peptidyl linker (¹³³GSGGGS¹³⁸) to human interleukin 2 receptor α-chain fragment (139-303), produced in Chinese hamster ovary (CHO) cells, glycosylated; human interleukin 2 (IL-2) (75-133)-peptide [Cys¹²⁵(51)>Ser]-mutant (1-59), fused via a G2 peptide linker (60-61) to human interleukin 2 (IL-2) (4-74)-peptide (62-132) and via a GSG₃S peptide linker (133-138) to human interleukin 2 receptor α-chain (IL2R subunit alpha, IL2Rα, IL2RA) (1-165)-peptide (139-303), produced in Chinese hamster ovary (CHO) cells, glycoform alfa. The amino acid sequence of nemvaleukin alfa is given in SEQ ID NO:6. In some embodiments, nemvaleukin alfa exhibits the following post-translational modifications: disulfide bridges at positions: 31-116, 141-285, 184-242, 269-301, 166-197 or 166-199, 168-199 or 168-197 (using the numbering in SEQ ID NO:6), and glycosylation sites at positions: N187, N206, T212 using the numbering in SEQ ID NO:6. The preparation and properties of nemvaleukin alfa, as well as additional alternative forms of IL-2 suitable for use in the invention, is described in U.S. Patent Application Publication No. US 2021/0038684 A1 and U.S. Pat. No. 10,183,979, the disclosures of which are incorporated by reference herein. In some embodiments, an IL-2 form suitable for use in the invention is a protein having at least 80%, at least 90%, at least 95%, or at least 90% sequence identity to SEQ ID NO:6. In some embodiments, an IL-2 form suitable for use in the invention has the amino acid sequence given in SEQ ID NO:6 or conservative amino acid substitutions thereof. In some embodiments, an IL-2 form suitable for use in the invention is a fusion protein comprising amino acids 24-452 of SEQ ID NO:7, or variants, fragments, or derivatives thereof. In some embodiments, an IL-2 form suitable for use in the invention is a fusion protein comprising an amino acid sequence having at least 80%, at least 90%, at least 95%, or at least 90% sequence identity to amino acids 24-452 of SEQ ID NO:7, or variants, fragments, or derivatives thereof. Other IL-2 forms suitable for use in the present invention are described in U.S. Pat. No. 10,183,979, the disclosures of which are incorporated by reference herein. Optionally, in some embodiments, an IL-2 form suitable for use in the invention is a fusion protein comprising a first fusion partner that is linked to a second fusion partner by a mucin domain polypeptide linker, wherein the first fusion partner is IL-1Rα or a protein having at least 98% amino acid sequence identity to IL-1Rα and having the receptor antagonist activity of IL-Ra, and wherein the second fusion partner comprises all or a portion of an immunoglobulin comprising an Fc region, wherein the mucin domain polypeptide linker comprises SEQ ID NO:8 or an amino acid sequence having at least 90% sequence identity to SEQ ID NO:8 and wherein the half-life of the fusion protein is improved as compared to a fusion of the first fusion partner to the second fusion partner in the absence of the mucin domain polypeptide linker.

TABLE 2
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH LRPRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRDLISNINV IVLELKGSET TFMCEYADET ATIVEFLNRW | 120 |
| | ITFSQSIIST LT | 132 |
| SEQ ID NO: 5 | APTSSSTKKT QLQLEHLLLD LQMILNGINN YKNPKLTRML TFKFYMPKKA TELKHLQCLE | 60 |
| IL-2 form | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 6 | SKNFHLRPRD LISNINVIVL ELKGSETTFM CEYADETATI VEFLNRWITF SQSIISTLTG | 60 |
| Nemvaleukin alfa | GSSSTKKTQL QLEHLLLDLQ MILNGINNYK NPKLTRMLTF KFYMPKKATE LKHLQCLEEE | 120 |
| | LKPLEEVLNL AQGSGGGSEL CDDDPPEIPE ATFKAMAYKE GTMLNCECKR GFRRIKSGSL | 180 |
| | YMLCTGNSSH SSWDNQCQCT SSATRNTTKQ VTPQPEEQKE RKTTEMQSPM QPVDQASLPG | 240 |
| | ECREPPPWEN EATERIYHFV VGQMVYYQCV QGYRALHRGP AESVCKMTHG KTRWTQPQLI | 300 |
| | CTG | 303 |
| SEQ ID NO: 7 | MDAMKRGLCC VLLLCGAVFV SARRPSGRKS SKMQAFRIWD VNQKTFYLRN NQLVAGYLQG | 60 |
| IL-2 form | PNVNLEEKID VVPIEPHALF LGIHGGKMCL SCVKSGDETR LQLEAVNITD LSENRKQDKR | 120 |
| | FAFIRSDSGP TTSFESAACP GWFLCTAMEA DQPVSLENMP DEGVMVTKFY FQEDESGSGG | 180 |
| | ASSESSASSD GPHPVITESR ASSESSASSD GPHPVITESR EPKSSDKTHT CPPCPAPELL | 240 |
| | GGPSVFLFPP KPKDTLMISR TPEVTCVVVD VSHEDPEVKF NWYVDGVEVH NAKTKPREEQ | 300 |
| | YNSTYRVVSV LTVLHQDWLN GKEYKCKVSN KALPAPIEKT ISKAKGQPRE PQVYTLPPSR | 360 |
| | EEMTKNQVSL TCLVKGFYPS DIAVEWESNG QPENNYKTTP PVLDSDGSFF LYSKLTVDKS | 420 |
| | RWQQGNVFSC SVMHEALHNH YTQKSLSLSP GK | 452 |
| SEQ ID NO: 8 | SESSASSDGP HPVITP | 16 |
| mucin domain | | |
| polypeptide | | |
| SEQ ID NO: 9 | MHKCDITLQE IIKTLNSLTE QKTLCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTL ENFLERLKTI | 120 |
| human IL-4 | MREKYSKCSS | 130 |
| (rhIL-4) | | |
| SEQ ID NO: 10 | MDCDIEGKDG KQYESVLMVS IDQLLDSMKE IGSNCLNNEF NFFKRHICDA NKEGMFLFRA | 60 |
| recombinant | ARKLRQFLKM NSTGDFDLHL LKVSEGTTIL LNCTGQVKGR KPAALGEAQP TKSLEENKSL | 120 |
| human IL-7 | KEQKKLNDLC FLKRLLQEIK TCWNKILMGT KEH | 153 |
| (rhIL-7) | | |
| SEQ ID NO: 11 | MNWVNVISDL KKIEDLIQSM HIDATLYTES DVHPSCKVTA MKCFLLELQV ISLESGDASI | 60 |
| recombinant | EDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 12 | MQDRHMIRMR QLIDIVDQLK NYVNDIVPEF LPAPEDVETN CEWSAFSCFQ KAQLKSANTG | 60 |
| recombinant | NNERIINVSI KKLKRKPPST NAGRRQKHRL TCPSCDSYEK KPPKEFLERF KSLLQKMIHQ | 120 |
| human IL-21 | ELSSRTHGSE DS | 132 |
| (rhIL-21) | | |

In some embodiments, an IL-2 form suitable for use in the invention includes a antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the antibody cytokine engrafted protein preferentially expands T effector cells over regulatory T cells. In an embodiment, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the IL-2 molecule is a mutein, and wherein the antibody cytokine engrafted protein preferentially expands T effector cells over regulatory T cells. In an embodiment, the IL-2 regimen comprises administration of an antibody described in U.S. Patent Application Publication No. US 2020/0270334 A1, the disclosures of which are incorporated by reference herein. In an embodiment, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the IL-2 molecule is a mutein, wherein the antibody cytokine engrafted protein preferentially expands T effector cells over regulatory T cells, and wherein the antibody further comprises an IgG class heavy chain and an IgG class light chain selected from the group consisting of: a IgG class light chain comprising SEQ ID NO:39 and a IgG class heavy chain comprising SEQ ID NO:38; a IgG class light chain comprising SEQ ID NO:37 and a IgG class heavy chain comprising SEQ ID NO:29; a IgG class light chain comprising SEQ ID NO:39 and a IgG class heavy chain comprising SEQ ID NO:29; and a IgG class light chain comprising SEQ ID NO:37 and a IgG class heavy chain comprising SEQ ID NO:38.

In an embodiment, an IL-2 molecule or a fragment thereof is engrafted into HCDR1 of the V_{H}, wherein the IL-2 molecule is a mutein. In an embodiment, an IL-2 molecule or a fragment thereof is engrafted into HCDR2 of the V_{H}, wherein the IL-2 molecule is a mutein. In an embodiment, an IL-2 molecule or a fragment thereof is engrafted into HCDR3 of the V_{H}, wherein the IL-2 molecule is a mutein. In an embodiment, an IL-2 molecule or a fragment thereof is engrafted into LCDR1 of the V_{L}, wherein the IL-2 molecule is a mutein. In an embodiment, an IL-2 molecule or a fragment thereof is engrafted into LCDR2 of the V_{L}, wherein the IL-2 molecule is a mutein. In an embodiment, an IL-2 molecule or a fragment thereof is engrafted into LCDR3 of the V_{L}, wherein the IL-2 molecule is a mutein.

The insertion of the IL-2 molecule can be at or near the N-terminal region of the CDR, in the middle region of the CDR or at or near the C-terminal region of the CDR. In some embodiments, the antibody cytokine engrafted protein comprises an IL-2 molecule incorporated into a CDR, wherein the IL2 sequence does not frameshift the CDR sequence. In some embodiments, the antibody cytokine engrafted protein comprises an IL-2 molecule incorporated into a CDR, wherein the IL-2 sequence replaces all or part of a CDR sequence. The replacement by the IL-2 molecule can be the N-terminal region of the CDR, in the middle region of the CDR or at or near the C-terminal region the CDR. A replacement by the IL-2 molecule can be as few as one or two amino acids of a CDR sequence, or the entire CDR sequences.

In some embodiments, an IL-2 molecule is engrafted directly into a CDR without a peptide linker, with no additional amino acids between the CDR sequence and the IL-2 sequence. In some embodiments, an IL-2 molecule is engrafted indirectly into a CDR with a peptide linker, with one or more additional amino acids between the CDR sequence and the IL-2 sequence.

In some embodiments, the IL-2 molecule described herein is an IL-2 mutein. In some instances, the IL-2 mutein comprising an R67A substitution. In some embodiments, the IL-2 mutein comprises the amino acid sequence SEQ ID NO:14 or SEQ ID NO:15. In some embodiments, the IL-2 mutein comprises an amino acid sequence in Table 1 in U.S. Patent Application Publication No. US 2020/0270334 At, the disclosure of which is incorporated by reference herein.

In an embodiment, the antibody cytokine engrafted protein comprises an HCDR1 selected from the group consisting of SEQ ID NO:16, SEQ ID NO:19, SEQ ID NO:22 and SEQ ID NO:25. In an embodiment, the antibody cytokine engrafted protein comprises an HCDR1 selected from the group consisting of SEQ ID NO:7, SEQ ID NO:10, SEQ ID NO:13 and SEQ ID NO:16. In an embodiment, the antibody cytokine engrafted protein comprises an HCDR1 selected from the group consisting of HCDR2 selected from the group consisting of SEQ ID NO:17, SEQ ID NO:20, SEQ ID NO:23, and SEQ ID NO:26. In an embodiment, the antibody cytokine engrafted protein comprises an HCDR3 selected from the group consisting of SEQ ID NO:18, SEQ ID NO:21, SEQ ID NO:24, and SEQ ID NO:27. In an embodiment, the antibody cytokine engrafted protein comprises a V_{H} region comprising the amino acid sequence of SEQ ID NO:28. In an embodiment, the antibody cytokine engrafted protein comprises a heavy chain comprising the amino acid sequence of SEQ ID NO:29. In an embodiment, the antibody cytokine engrafted protein comprises a V_{L} region comprising the amino acid sequence of SEQ ID NO:36. In an embodiment, the antibody cytokine engrafted protein comprises a light chain comprising the amino acid sequence of SEQ ID NO:37. In an embodiment, the antibody cytokine engrafted protein comprises a V_{H} region comprising the amino acid sequence of SEQ ID NO:28 and a V_{L} region comprising the amino acid sequence of SEQ ID NO:36. In an embodiment, the antibody cytokine engrafted protein comprises a heavy chain region comprising the amino acid sequence of SEQ ID NO:29 and a light chain region comprising the amino acid sequence of SEQ ID NO:37. In an embodiment, the antibody cytokine engrafted protein comprises a heavy chain region comprising the amino acid sequence of SEQ ID NO:29 and a light chain region comprising the amino acid sequence of SEQ ID NO:39. In an embodiment, the antibody cytokine engrafted protein comprises a heavy chain region comprising the amino acid sequence of SEQ ID NO:38 and a light chain region comprising the amino acid sequence of SEQ ID NO:37. In an embodiment, the antibody cytokine engrafted protein comprises a heavy chain region comprising the amino acid sequence of SEQ ID NO:38 and a light chain region comprising the amino acid sequence of SEQ ID NO:39. In an embodiment, the antibody cytokine engrafted protein comprises IgG.IL2F71A.H1 or IgG.IL2R67A.H1 of U.S. Patent Application Publication No. 2020/0270334 A1, or variants, derivatives, or fragments thereof, or conservative amino acid substitutions thereof, or proteins with at least 80%, at least 90%, at least 95%, or at least 98% sequence identity thereto. In an embodiment, the antibody components of the antibody cytokine engrafted protein described herein comprise immunoglobulin sequences, framework sequences, or CDR sequences of palivizumab. In some embodiments, the antibody cytokine engrafted protein described herein has a longer serum half-life that a wild-type IL-2 molecule such as, but not limited to, aldesleukin or a comparable molecule. In an embodiment, the antibody cytokine engrafted protein described herein has a sequence as set forth in Table 3.

TABLE 3
Sequences of exemplary palivizumab antibody-IL-2 engrafted proteins.

| Identifier | Sequence (One-Letter Amino Acid Symbols) | |
|---|---|---|
| SEQ ID NO: 13 | MYRMQLLSCI ALSLALVTNS APTSSSTKKT QLQLEHLLLD LQMILNGINN YKNPKLTRML | 60 |
| IL-2 | TFKFYMPKKA TELKHLQCLE EELKPLEEVL NLAQSKNFHL RPRDLISNIN VIVLELKGSE | 120 |
| | TTFMCEYADE TATIVEFLNR WITFCQSIIS TLT | 153 |
| SEQ ID NO: 14 | APTSSSTKKT QLQLEHLLLD LQMILNGINN YKNPKLTAML TFKFYMPKKA TELKHLQCLE | 60 |
| IL-2 mutein | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 15 | APTSSSTKKT QLQLEHLLLD LQMILNGINN YKNPKLTRML TAKFYMPKKA TELKHLQCLE | 60 |
| IL-2 mutein | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 16 | GFSLAPTSSS TKKTQLQLEH LLLDLQMILN GINNYKNPKL TAMLTFKFYM PKKATELKHL | 60 |
| HCDR1_IL-2 | QCLEEELKPL EEVLNLAQSK NFHLRPRDLI SNINVIVLEL KGSETTFMCE YADETATIVE | 120 |
| | FLNRWITFCQ SIISTLTSTS GMSVG | 145 |
| SEQ ID NO: 17 | DIWWDDKKDY NPSLKS | 16 |
| HCDR2 | | |
| SEQ ID NO: 18 | SMITNWYFDV | 10 |
| HCDR3 | | |
| SEQ ID NO: 19 | APTSSSTKKT QLQLEHLLLD LQMILNGINN YKNPKLTAML TFKFYMPKKA TELKHLQCLE | 60 |
| HCDR1_IL-2 kabat | EELKPLEEVL NLAQSKNFHL RPRDLISNIN VIVLELKGSE TTFMCEYADE TATIVEFLNR | 120 |
| | WITFCQSIIS TLTSTSGMSV G | 141 |
| SEQ ID NO: 20 | DIWWDDKKDY NPSLKS | 16 |
| HCDR2 kabat | | |
| SEQ ID NO: 21 | SMITNWYFDV | 10 |
| HCDR3 kabat | | |
| SEQ ID NO: 22 | GFSLAPTSSS TKKTQLQLEH LLLDLQMILN GINNYKNPKL TAMLTFKFYM PKKATELKHL | 60 |
| HCDR1_IL-2 | QCLEEELKPL EEVLNLAQSK NFHLRPRDLI SNINVIVLEL KGSETTFMCE YADETATIVE | 120 |
| clothia | FLNRWITFCQ SIISTLTSTS GM | 142 |
| SEQ ID NO: 23 | WWDDK | 5 |
| HCDR2 clothia | | |
| SEQ ID NO: 24 | SMITNWYFDV | 10 |
| HCDR3 clothia | | |
| SEQ ID NO: 25 | GFSLAPTSSS TKKTQLQLEH LLLDLQMILN GINNYKNPKL TAMLTFKFYM PKKATELKHL | 60 |
| HCDR1_IL-2 IMGT | QCLEEELKPL EEVLNLAQSK NFHLRPRDLI SNINVIVLEL KGSETTFMCE YADETATIVE | 120 |
| | FLNRWITFCQ SIISTLTSTS GMS | 143 |
| SEQ ID NO: 26 | IWWDDKK | 7 |
| HCDR2 IMGT | | |
| SEQ ID NO: 27 | ARSMITNWYF DV | 12 |
| HCDR3 IMGT | | |
| SEQ ID NO: 28 | QVTLRESGPA LVKPTQTLTL TCTFSGFSLA PTSSSTKKTQ LQLEHLLLDL QMILNGINNY | 60 |
| V_{H} | KNPKLTAMLT FKFYMPKKAT ELKHLQCLEE ELKPLEEVLN LAQSKNFHLR PRDLISNINV | 120 |
| | IVLELKGSET TFMCEYADET ATIVEFLNRW ITFCQSIIST LTSTSGMSVG WIRQPPGKAL | 180 |
| | EWLADIWWDD KKDYNPSLKS RLTISKDTSK NQVVLKVTNM DPADTATYYC ARSMITNWYF | 240 |
| | DVWGAGTTVT VSS | 253 |
| SEQ ID NO: 29 | QMILNGINNY KNPKLTAMLT FKFYMPKKAT ELKHLQCLEE ELKPLEEVLN LAQSKNFHLR | 60 |
| Heavy chain | PRDLISNINV IVLELKGSET TFMCEYADET ATIVEFLNRW ITFCQSIIST LTSTSGMSVG | 120 |
| | WIRQPPGKAL EWLADIWWDD KKDYNPSLKS RLTISKDTSK NQVVLKVTNM DPADTATYYC | 180 |
| | ARSMITNWYF DVWGAGTTVT VSSASTKGPS VFPLAPSSKS TSGGTAALGC LVKDYFPEPV | 240 |
| | TVSWNSGALT SGVHTFPAVL QSSGLYSLSS VVTVPSSSLG TQTYICNVNH KPSNTKVDKR | 300 |
| | VEPKSCDKTH TCPPCPAPEL LGGPSVFLFP PKPKDTLMIS RTPEVTCVVV AVSHEDPEVK | 360 |
| | FNWYVDGVEV HNAKTKPREE QYNSTYRVVS VLTVLHQDWL NGKEYKCKVS NKALAAPIEK | 420 |
| | TISKAKGQPR EPQVYTLPPS REEMTKNQVS LTCLVKGFYP SDIAVEWESN GQPENNYKTT | 480 |
| | PPVLDSDGSF FLYSKLTVDK SRWQQGNVFS CSVMHEALHN HYTQKSLSLS PGK | 533 |
| SEQ ID NO: 30 | KAQLSVGYMH | 10 |
| LCDR1 kabat | | |
| SEQ ID NO: 31 | DTSKLAS | 7 |
| LCDR2 kabat | | |
| SEQ ID NO: 32 | FQGSGYPFT | 9 |
| LCDR3 kabat | | |
| SEQ ID NO: 33 | QLSVGY | 6 |
| LCDR1 chothia | | |
| SEQ ID NO: 34 | DTS | 3 |
| LCDR2 chothia | | |
| SEQ ID NO: 35 | GSGYPF | 6 |
| LCDR3 chothia | | |
| SEQ ID NO: 36 | DIQMTQSPST LSASVGDRVT ITCKAQLSVG YMHWYQQKPG KAPKLLIYDT SKLASGVPSR | 60 |
| V_{L} | FSGSGSGTEF TLTISSLQPD DFATYYCFQG SGYPFTFGGG TKLEIK | 106 |
| SEQ ID NO: 37 | DIQMTQSPST LSASVGDRVT ITCKAQLSVG YMHWYQQKPG KAPKLLIYDT SKLASGVPSR | 60 |
| Light chain | FSGSGSGTEF TLTISSLQPD DFATYYCFQG SGYPFTFGGG TKLEIKRTVA APSVFIFPPS | 120 |
| | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 38 | QVTLRESGPA LVKPTQTLTL TCTFSGFSLA PTSSSTKKTQ LQLEHLLLDL QMILNGINNY | 60 |
| Light chain | KNPKLTRMLT AKFYMPKKAT ELKHLQCLEE ELKPLEEVLN LAQSKNFHLR PRDLISNINV | 120 |
| | IVLELKGSET TFMCEYADET ATIVEFLNRW ITFCQSIIST LTSTSGMSVG WIRQPPGKAL | 180 |
| | EWLADIWWDD KKDYNPSLKS RLTISKDTSK NQVVLKVTNM DPADTATYYC ARSMITNWYF | 240 |
| | DVWGAGTTVT VSSASTKGPS VFPLAPSSKS TSGGTAALGC LVKDYFPEPV TVSWNSGALT | 300 |
| | SGVHTFPAVL QSSGLYSLSS VVTVPSSSLG TQTYICNVNH KPSNTKVDKR VEPKSCDKTH | 360 |
| | TCPPCPAPEL LGGPSVFLFP PKPKDTLMIS RTPEVTCVVV AVSHEDPEVK FNWYVDGVEV | 420 |
| | HNAKTKPREE QYNSTYRVVS VLTVLHQDWL NGKEYKCKVS NKALAAPIEK TISKAKGQPR | 480 |
| | EPQVYTLPPS REEMTKNQVS LTCLVKGFYP SDIAVEWESN GQPENNYKTT PFVLDSDGSF | 540 |
| | FLYSKLTVDK SRWQQGNVFS CSVMHEALHN HYTQKSLSLS PGK | 583 |
| SEQ ID NO: 39 | DIQMTQSPST LSASVGDRVT ITCKAQLSVG YMHWYQQKPG KAPKLLIYDT SKLASGVPSR | 60 |
| Light chain | FSGSGSGTEF TLTISSLQPD DFATYYCFQG SGYPFTFGGG TKLEIKRTVA APSVFIFPPS | 120 |
| | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, NJ, USA (Cat. No. CYT-211) and ThermoFisher Scientific, Inc., Waltham, MA, USA (human IL-15 recombinant protein, Cat. No. Gibco CTP0043). The amino acid sequence of recombinant human IL-4 suitable for use in the invention is given in Table 2 (SEQ ID NO:9).

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, NJ, USA (Cat. No. CYT-211) and ThermoFisher Scientific, Inc., Waltham, MA, USA (human IL-15 recombinant protein, Cat. No. Gibco CTP0043). The amino acid sequence of recombinant human IL-4 suitable for use in the invention is given in Table 2 (SEQ ID NO:5).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, NJ, USA (Cat. No. CYT-254) and ThermoFisher Scientific, Inc., Waltham, MA, USA (human IL-15 recombinant protein, Cat. No. Gibco PHC0071). The amino acid sequence of recombinant human IL-7 suitable for use in the invention is given in Table 2 (SEQ ID NO:6).

The term “IL-15” (also referred to herein as “IL15”) refers to the T cell growth factor known as interleukin-15 and includes all forms of IL-2 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). The amino acid sequence of recombinant human IL-15 suitable for use in the invention is given in Table 2 (SEQ ID NO:7).

The term “IL-21” (also referred to herein as “IL21”) refers to the pleiotropic cytokine protein known as interleukin-21 and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4⁺ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, NJ, USA (Cat. No. CYT-408-b) and ThermoFisher Scientific, Inc., Waltham, MA, USA (human IL-21 recombinant protein, Cat. No. 14-8219-80). The amino acid sequence of recombinant human IL-21 suitable for use in the invention is given in Table 2 (SEQ ID NO:8).

When “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the tumor infiltrating lymphocytes (e.g. secondary TILs or genetically modified cytotoxic lymphocytes) described herein may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹¹, or 10⁹ to 10¹⁰ cells/kg body weight), including all integer values within those ranges. TILs (including in some cases, genetically modified cytotoxic lymphocytes) compositions may also be administered multiple times at these dosages. The TILs (including, in some cases, genetically engineered TILs) can be administered by using infusion techniques that are commonly known in immunotherapy (see, e.g., Rosenberg, et al., New Eng. J Med. 1988, 319, 1676). The optimal dosage and treatment regime for a particular patient can readily be determined by one skilled in the art of medicine by monitoring the patient for signs of disease and adjusting the treatment accordingly.

The term “hematological malignancy”, “hematologic malignancy” or terms of correlative meaning refer to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), multiple myeloma, acute monocytic leukemia (AMoL), Hodgkin's lymphoma, and non-Hodgkin's lymphomas. The term “B cell hematological malignancy” refers to hematological malignancies that affect B cells.

The term “liquid tumor” refers to an abnormal mass of cells that is fluid in nature. Liquid tumor cancers include, but are not limited to, leukemias, myelomas, and lymphomas, as well as other hematological malignancies. TILs obtained from liquid tumors may also be referred to herein as marrow infiltrating lymphocytes (MILs). TILs obtained from liquid tumors, including liquid tumors circulating in peripheral blood, may also be referred to herein as PBLs. The terms MIL, TIL, and PBL are used interchangeably herein and differ only based on the tissue type from which the cells are derived.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et at, Cancer Res., 2012, 72, 2473. Although tumors express antigens that should be recognized by T cells, tumor clearance by the immune system is rare because of immune suppression by the microenvironment.

In an embodiment, the invention includes a method of treating a cancer with a population of TILs, wherein a patient is pre-treated with non-myeloablative chemotherapy prior to an infusion of TILs according to the invention. In some embodiments, the population of TILs may be provided wherein a patient is pre-treated with nonmyeloablative chemotherapy prior to an infusion of TILs according to the present invention. In an embodiment, the non-myeloablative chemotherapy is cyclophosphamide 60 mg/kg/d for 2 days (days 27 and 26 prior to TIL infusion) and fludarabine 25 mg/m2/d for 5 days (days 27 to 23 prior to TIL infusion). In an embodiment, after non-myeloablative chemotherapy and TIL infusion (at day 0) according to the invention, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the TILs of the invention.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

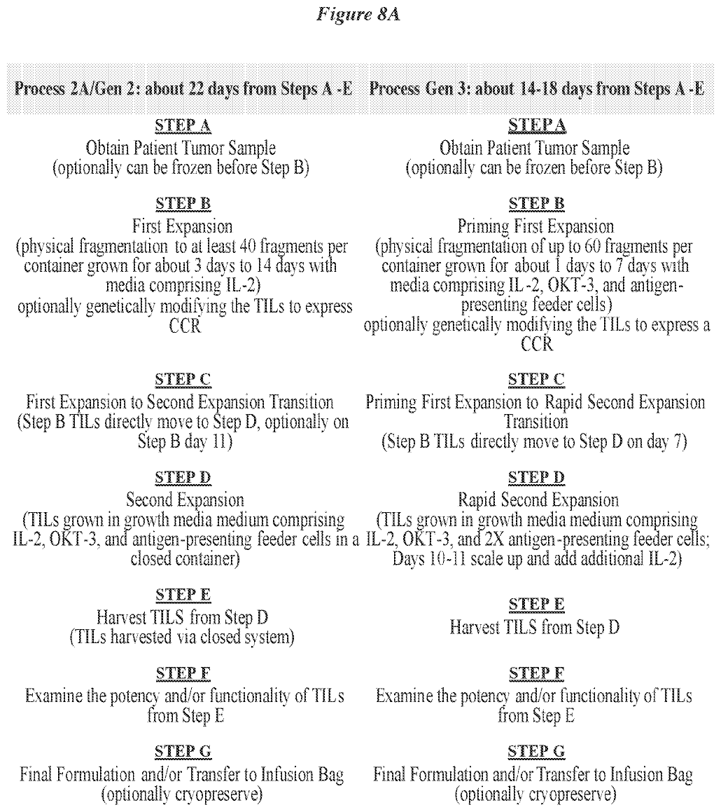

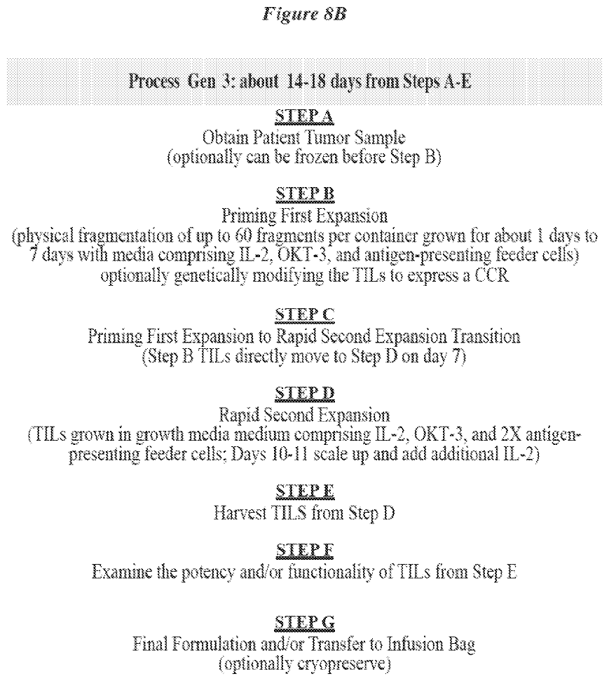

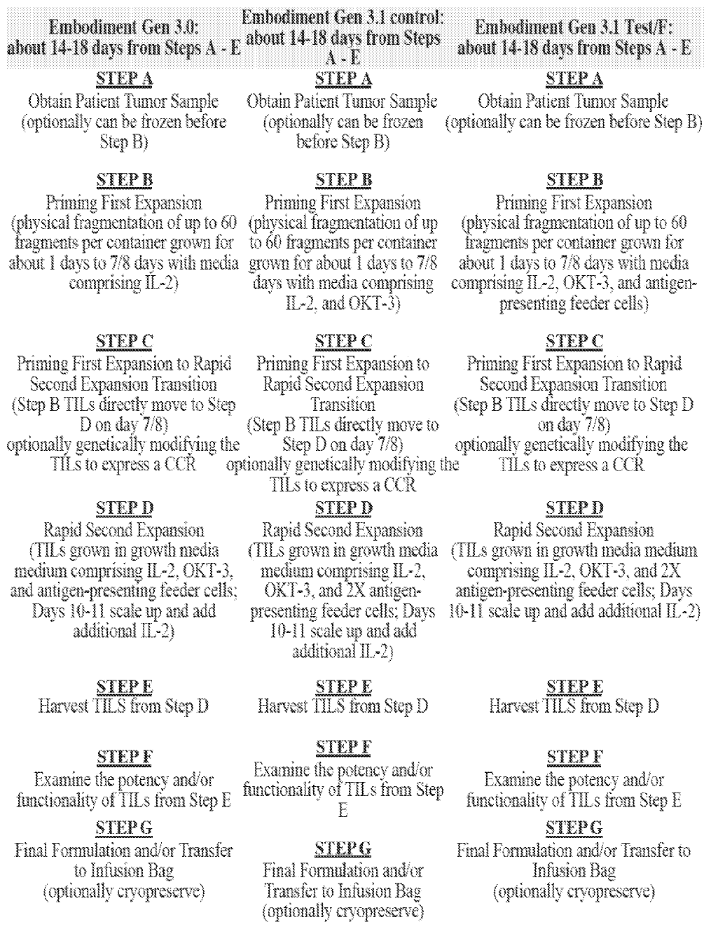

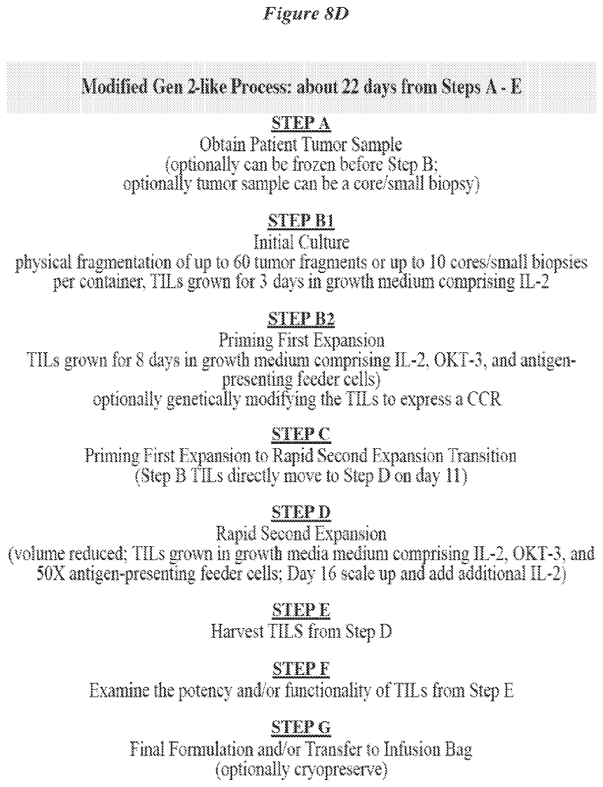

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs, expanded TILs (“REP TILs”) as well as “reREP TILs” as discussed herein. reREP TILs can include for example second expansion TILs or second additional expansion TILs (such as, for example, those described in Step D of FIG. 8, including TILs referred to as reREP TILs).

TILs can generally be defined either biochemically, using cell surface markers, or functionally, by their ability to infiltrate tumors and effect treatment. TILs can be generally categorized by expressing one or more of the following biomarkers: CD4, CD8, TCR ap, CD27, CD28, CD56, CCR7, CD45Ra, CD95, PD-1, and CD25. Additionally, and alternatively, TILs can be functionally defined by their ability to infiltrate solid tumors upon reintroduction into a patient. TILs may further be characterized by potency—for example, TILs may be considered potent if, for example, interferon (IFN) release is greater than about 50 pg/mL, greater than about 100 pg/mL, greater than about 150 pg/mL, or greater than about 200 pg/mL.

The term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide.

The term “RNA” defines a molecule comprising at least one ribonucleotide residue. The term “ribonucleotide” defines a nucleotide with a hydroxyl group at the 2′ position of a b-D-ribofuranose moiety. The term RNA includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Nucleotides of the RNA molecules described herein may also comprise non-standard nucleotides, such as non-naturally occurring nucleotides or chemically synthesized nucleotides or deoxynucleotides. These altered RNAs can be referred to as analogs or analogs of naturally-occurring RNA.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in therapeutic compositions of the invention is contemplated. Additional active pharmaceutical ingredients, such as other drugs, can also be incorporated into the described compositions and methods.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The term “antigen” refers to a substance that induces an immune response. In some embodiments, an antigen is a molecule capable of being bound by an antibody or a TCR if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to certain receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion” or “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies. In an embodiment, a scFv protein domain comprises a V_{H} portion and a V_{L} portion. A scFv molecule is denoted as either V_{L}-L-V_{H} if the V_{L} domain is the N-terminal part of the scFv molecule, or as V_{H}-L-V_{L} if the V_{H} domain is the N-terminal part of the scFv molecule. Methods for making scFv molecules and designing suitable peptide linkers are described in U.S. Pat. Nos. 4,704,692, 4,946,778, R. Raag and M. Whitlow, “Single Chain Fvs.” FASEB Vol 9:73-80 (1995) and R. E. Bird and B. W. Walker, Single Chain Antibody Variable Regions, TIBTECH, Vol 9: 132-137 (1991), the disclosures of which are incorporated by reference herein.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In an embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, including a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to another therapeutic moiety, which can be conjugated to antibodies described herein using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596. The antibodies described herein may also be modified to employ any Fc variant which is known to impart an improvement (e.g., reduction) in effector function and/or FcR binding. The Fc variants may include, for example, any one of the amino acid substitutions disclosed in International Patent Application Publication Nos. WO 1988/07089 A1, WO 1996/14339 A1, WO 1998/05787 A1, WO 1998/23289 A1, WO 1999/51642 A1, WO 99/58572 A1, WO 2000/09560 A2, WO 2000/32767 A1, WO 2000/42072 A2, WO 2002/44215 A2, WO 2002/060919 A2, WO 2003/074569 A2, WO 2004/016750 A2, WO 2004/029207 A2, WO 2004/035752 A2, WO 2004/063351 A2, WO 2004/074455 A2, WO 2004/099249 A2, WO 2005/040217 A2, WO 2005/070963 A1, WO 2005/077981 A2, WO 2005/092925 A2, WO 2005/123780 A2, WO 2006/019447 A1, WO 2006/047350 A2, and WO 2006/085967 A2; and U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784; the disclosures of which are incorporated by reference herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., Proc. Nat. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀)alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384 and U.S. Pat. No. 5,824,778, the disclosures of each of which are incorporated by reference herein.

The term “biosimilar” means a biological product, including a monoclonal antibody or protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference IL-2 protein is aldesleukin (PROLEUKIN), a protein approved by drug regulatory authorities with reference to aldesleukin is a “biosimilar to” aldesleukin or is a “biosimilar thereof” of aldesleukin. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

### III. Gen 2 TIL Manufacturing Processes

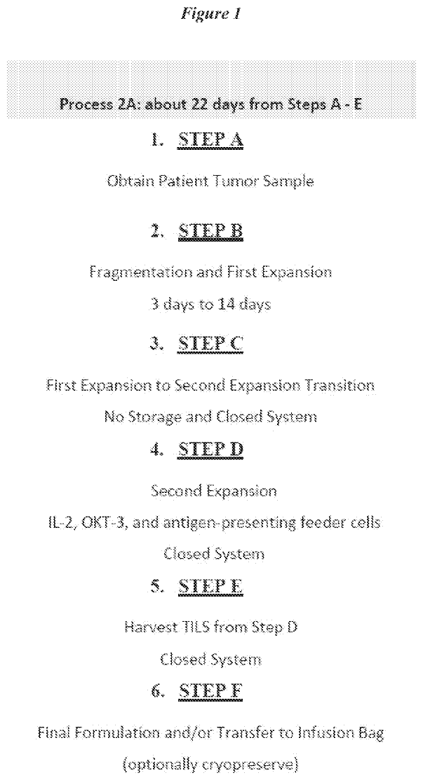

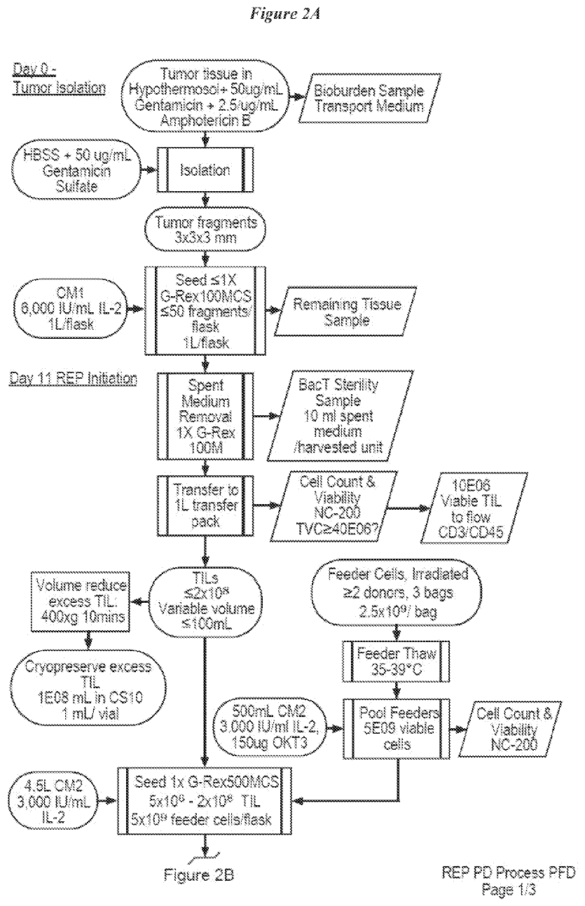

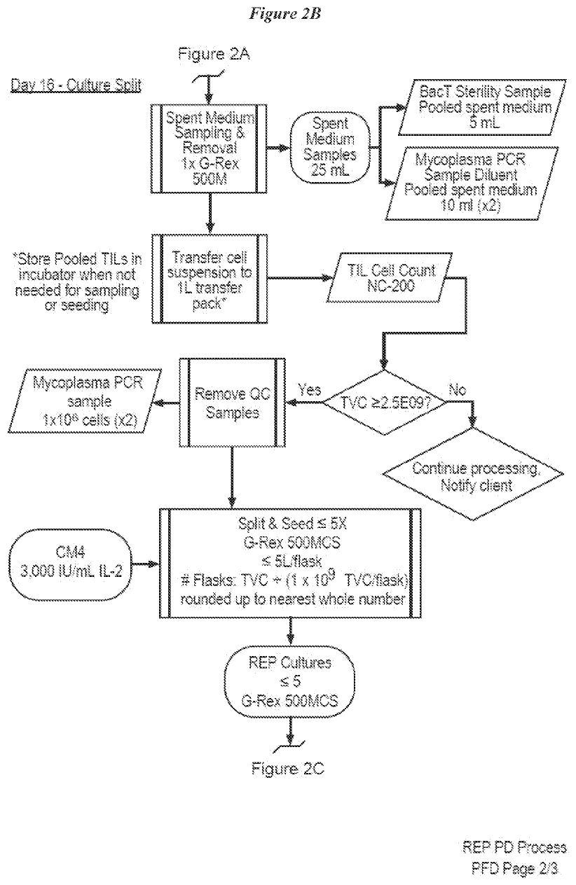

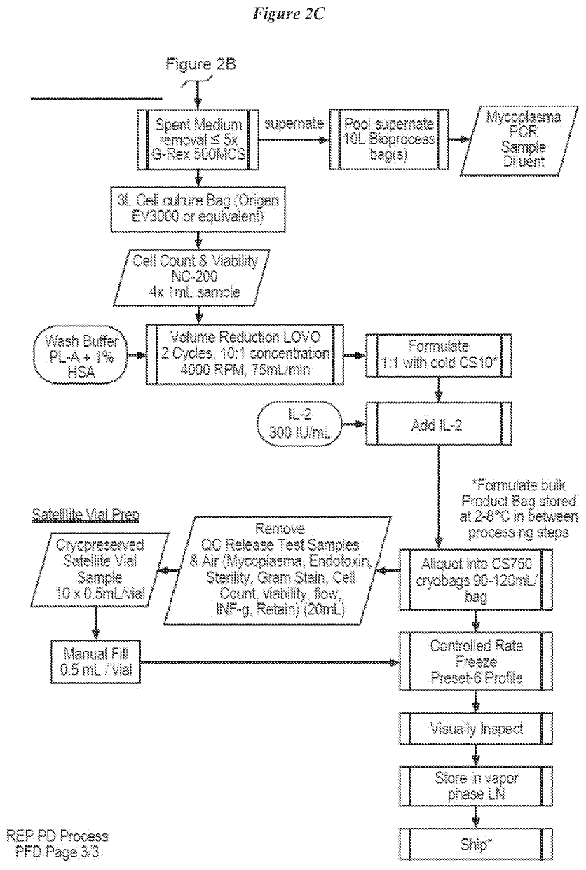

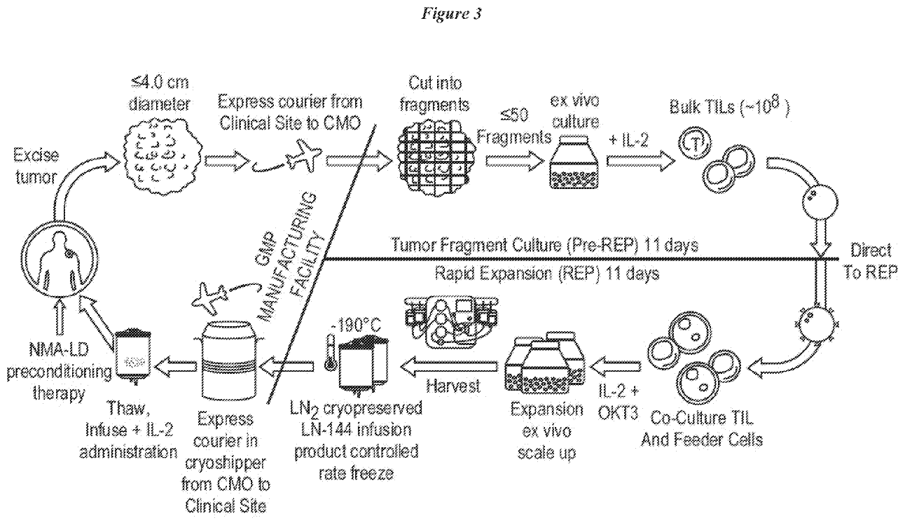

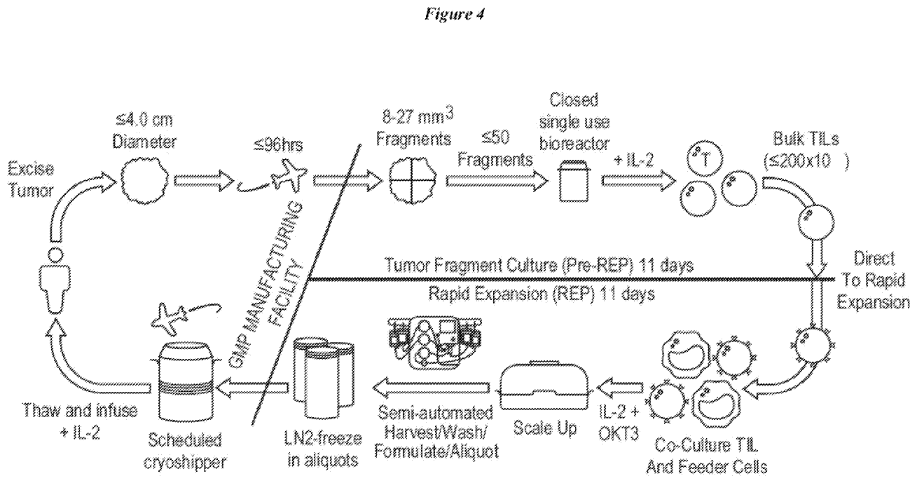

An exemplary family of TIL processes known as Gen 2 (also known as process 2A) containing some of these features is depicted in FIGS. 1 and 2. An embodiment of Gen 2 is shown in FIG. 2.

As discussed herein, the present invention can include a step relating to the restimulation of cryopreserved TILs to increase their metabolic activity and thus relative health prior to transplant into a patient, and methods of testing said metabolic health. As generally outlined herein, TILs are generally taken from a patient sample and manipulated to expand their number prior to transplant into a patient. In some embodiments, the TILs may be optionally genetically manipulated as discussed below.

In some embodiments, the TILs may be cryopreserved. Once thawed, they may also be restimulated to increase their metabolism prior to infusion into a patient.

In some embodiments, the first expansion (including processes referred to as the pre-REP as well as processes shown in FIG. 1 as Step A) is shortened to 3 to 14 days and the second expansion (including processes referred to as the REP as well as processes shown in FIG. 1 as Step B) is shorted to 7 to 14 days, as discussed in detail below as well as in the examples and figures. In some embodiments, the first expansion (for example, an expansion described as Step B in FIG. 1) is shortened to 11 days and the second expansion (for example, an expansion as described in Step D in FIG. 1) is shortened to 11 days. In some embodiments, the combination of the first expansion and second expansion (for example, expansions described as Step B and Step D in FIG. 1) is shortened to 22 days, as discussed in detail below and in the examples and figures.

The “Step” Designations A, B, C, etc., below are in reference to FIG. 1 and in reference to certain embodiments described herein. The ordering of the Steps below and in FIG. 1 is exemplary and any combination or order of steps, as well as additional steps, repetition of steps, and/or omission of steps is contemplated by the present application and the methods disclosed herein.

### A. STEP A: Obtain Patient Tumor Sample

In general, TILs are initially obtained from a patient tumor sample and then expanded into a larger population for further manipulation as described herein, optionally cryopreserved, restimulated as outlined herein and optionally evaluated for phenotype and metabolic parameters as an indication of TIL health.

A patient tumor sample may be obtained using methods known in the art, generally via surgical resection, needle biopsy, core biopsy, small biopsy, or other means for obtaining a sample that contains a mixture of tumor and TIL cells. In some embodiments, multilesional sampling is used. In some embodiments, surgical resection, needle biopsy, core biopsy, small biopsy, or other means for obtaining a sample that contains a mixture of tumor and TIL cells includes multilesional sampling (i.e., obtaining samples from one or more tumor cites and/or locations in the patient, as well as one or more tumors in the same location or in close proximity). In general, the tumor sample may be from any solid tumor, including primary tumors, invasive tumors or metastatic tumors. The tumor sample may also be a liquid tumor, such as a tumor obtained from a hematological malignancy. The solid tumor may be of lung tissue. In some embodiments, useful TILs are obtained from non-small cell lung carcinoma (NSCLC).

Once obtained, the tumor sample is generally fragmented using sharp dissection into small pieces of between 1 to about 8 mm³, with from about 2-3 mm³ being particularly useful. In some embodiments, the TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

Tumor dissociating enzyme mixtures can include one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof.

In some embodiments, the dissociating enzymes are reconstituted from lyophilized enzymes. In some embodiments, lyophilized enzymes are reconstituted in an amount of sterile buffer such as HBSS.

In some instances, collagenase (such as animal free-type 1 collagenase) is reconstituted in 10 mL of sterile HBSS or another buffer. The lyophilized stock enzyme may be at a concentration of 2892 PZ U/vial. In some embodiments, collagenase is reconstituted in 5 mL to 15 mL buffer. In some embodiment, after reconstitution the collagenase stock ranges from about 100 PZ U/mL-about 400 PZ U/mL, e.g., about 100 PZ U/mL-about 400 PZ U/mL, about 100 PZ U/mL-about 350 PZ U/mL, about 100 PZ U/mL-about 300 PZ U/mL, about 150 PZ U/mL-about 400 PZ U/mL, about 100 PZ U/mL, about 150 PZ U/mL, about 200 PZ U/mL, about 210 PZ U/mL, about 220 PZ U/mL, about 230 PZ U/mL, about 240 PZ U/mL, about 250 PZ U/mL, about 260 PZ U/mL, about 270 PZ U/mL, about 280 PZ U/mL, about 289.2 PZ U/mL, about 300 PZ U/mL, about 350 PZ U/mL, or about 400 PZ U/mL.

In some embodiments, neutral protease is reconstituted in 1 mL of sterile HBSS or another buffer. The lyophilized stock enzyme may be at a concentration of 175 DMC U/vial. In some embodiments, after reconstitution the neutral protease stock ranges from about 100 DMC/mL-about 400 DMC/mL, e.g., about 100 DMC/mL-about 400 DMC/mL, about 100 DMC/mL-about 350 DMC/mL, about 100 DMC/mL-about 300 DMC/mL, about 150 DMC/mL-about 400 DMC/mL, about 100 DMC/mL, about 110 DMC/mL, about 120 DMC/mL, about 130 DMC/mL, about 140 DMC/mL, about 150 DMC/mL, about 160 DMC/mL, about 170 DMC/mL, about 175 DMC/mL, about 180 DMC/mL, about 190 DMC/mL, about 200 DMC/mL, about 250 DMC/mL, about 300 DMC/mL, about 350 DMC/mL, or about 400 DMC/mL.

In some embodiments, DNAse I is reconstituted in 1 mL of sterile HBSS or another buffer. The lyophilized stock enzyme was at a concentration of 4 KU/vial. In some embodiments, after reconstitution the DNase I stock ranges from about 1 KU/mL-10 KU/mL, e.g., about 1 KU/mL, about 2 KU/mL, about 3 KU/mL, about 4 KU/mL, about 5 KU/mL, about 6 KU/mL, about 7 KU/mL, about 8 KU/mL, about 9 KU/mL, or about 10 KU/mL.

In some embodiments, the stock of enzymes is variable and the concentrations may need to be determined. In some embodiments, the concentration of the lyophilized stock can be verified. In some embodiments, the final amount of enzyme added to the digest cocktail is adjusted based on the determined stock concentration.

In some embodiment, the enzyme mixture includes about 10.2-ul of neutral protease (0.36 DMC U/mL), 21.3 μL of collagenase (1.2 PZ/mL) and 250-ul of DNAse I (200 U/mL) in about 4.7 mL of sterile HBSS.

As indicated above, in some embodiments, the TILs are derived from solid tumors. In some embodiments, the solid tumors are not fragmented. In some embodiments, the solid tumors are not fragmented and are subjected to enzymatic digestion as whole tumors. In some embodiments, the tumors are digested in in an enzyme mixture comprising collagenase, DNase, and hyaluronidase. In some embodiments, the tumors are digested in in an enzyme mixture comprising collagenase, DNase, and hyaluronidase for 1-2 hours. In some embodiments, the tumors are digested in in an enzyme mixture comprising collagenase, DNase, and hyaluronidase for 1-2 hours at 37° C., 5% CO₂. In some embodiments, the tumors are digested in in an enzyme mixture comprising collagenase, DNase, and hyaluronidase for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture.

In some embodiments, the tumor is reconstituted with the lyophilized enzymes in a sterile buffer. In some embodiments, the buffer is sterile HBSS.

In some embodiments, the enzyme mixture comprises collagenase. In some embodiments, the collagenase is collagenase IV. In some embodiments, the working stock for the collagenase is a 100 mg/mL 10× working stock.

In some embodiments, the enzyme mixture comprises DNAse. In some embodiments, the working stock for the DNAse is a 10,000 IU/mL 10× working stock.

In some embodiments, the enzyme mixture comprises hyaluronidase. In some embodiments, the working stock for the hyaluronidase is a 10-mg/mL 10× working stock.

In some embodiments, the enzyme mixture comprises 10 mg/mL collagenase, 1000 IU/mL DNAse, and 1 mg/mL hyaluronidase.

In some embodiments, the enzyme mixture comprises 10 mg/mL collagenase, 500 IU/mL DNAse, and 1 mg/mL hyaluronidase.

In general, the harvested cell suspension is called a “primary cell population” or a “freshly harvested” cell population.

In some embodiments, fragmentation includes physical fragmentation, including for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from patients. In an embodiment, TILs can be initially cultured from enzymatic tumor digests and tumor fragments obtained from patients.

In some embodiments, where the tumor is a solid tumor, the tumor undergoes physical fragmentation after the tumor sample is obtained in, for example, Step A (as provided in FIG. 1). In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the tumor is fragmented and 10, 20, 30, 40 or more fragments or pieces are placed in each container for the first expansion. In some embodiments, the tumor is fragmented and 30 or 40 fragments or pieces are placed in each container for the first expansion. In some embodiments, the tumor is fragmented and 40 fragments or pieces are placed in each container for the first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumors are 1-4 mm×1-4 mm×1-4 mm. In some embodiments, the tumors are 1 mm×1 mm×1 mm. In some embodiments, the tumors are 2 mm×2 mm×2 mm. In some embodiments, the tumors are 3 mm×3 mm×3 mm. In some embodiments, the tumors are 4 mm×4 mm×4 mm.

In some embodiments, the tumors are resected in order to minimize the amount of hemorrhagic, necrotic, and/or fatty tissues on each piece. In some embodiments, the tumors are resected in order to minimize the amount of hemorrhagic tissue on each piece. In some embodiments, the tumors are resected in order to minimize the amount of necrotic tissue on each piece. In some embodiments, the tumors are resected in order to minimize the amount of fatty tissue on each piece.

In some embodiments, the tumor fragmentation is performed in order to maintain the tumor internal structure. In some embodiments, the tumor fragmentation is performed without preforming a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, the harvested cell suspension prior to the first expansion step is called a “primary cell population” or a “freshly harvested” cell population.

In some embodiments, cells can be optionally frozen after sample harvest and stored frozen prior to entry into the expansion described in Step B, which is described in further detail below, as well as exemplified in FIG. 1.

### 1. Pleural Effusion T-Cells and TILs

In some embodiments, the sample is a pleural fluid sample. In some embodiments, the source of the T-cells TILs for expansion according to the processes described herein is a pleural fluid sample. In some embodiments, the sample is a pleural effusion derived sample. In some embodiments, the source of the T-cells or TILs for expansion according to the processes described herein is a pleural effusion derived sample. See, for example, methods described in U.S. Patent Publication US 2014/0295426, incorporated herein by reference in its entirety for all purposes.

In some embodiments, any pleural fluid or pleural effusion suspected of and/or containing TILs can be employed. Such a sample may be derived from a primary or metastatic lung cancer, such as NSCLC or SCLC. In some embodiments, the sample may be secondary metastatic cancer cells which originated from another organ, e.g., breast, ovary, colon or prostate. In some embodiments, the sample for use in the expansion methods described herein is a pleural exudate. In some embodiments, the sample for use in the expansion methods described herein is a pleural transudate. Other biological samples may include other serous fluids containing TILs, including, e.g., ascites fluid from the abdomen or pancreatic cyst fluid. Ascites fluid and pleural fluids involve very similar chemical systems; both the abdomen and lung have mesothelial lines and fluid forms in the pleural space and abdominal spaces in the same matter in malignancies and such fluids in some embodiments contain TILs. In some embodiments, wherein the disclosure exemplifies pleural fluid, the same methods may be performed with similar results using ascites or other cyst fluids containing TILs.

In some embodiments, the pleural fluid is in unprocessed form, directly as removed from the patient. In some embodiments, the unprocessed pleural fluid is placed in a standard blood collection tube, such as an EDTA or Heparin tube, prior to the contacting step. In some embodiments, the unprocessed pleural fluid is placed in a standard CellSave® tube (Veridex) prior to the contacting step. In some embodiments, the sample is placed in the CellSave tube immediately after collection from the patient to avoid a decrease in the number of viable TILs. The number of viable TILs can decrease to a significant extent within 24 hours, if left in the untreated pleural fluid, even at 4° C. In some embodiments, the sample is placed in the appropriate collection tube within 1 hour, 5 hours, 10 hours, 15 hours, or up to 24 hours after removal from the patient. In some embodiments, the sample is placed in the appropriate collection tube within 1 hour, 5 hours, 10 hours, 15 hours, or up to 24 hours after removal from the patient at 4° C.

In some embodiments, the pleural fluid sample from the chosen subject may be diluted. In one embodiment, the dilution is 1:10 pleural fluid to diluent. In another embodiment, the dilution is 1:9 pleural fluid to diluent. In another embodiment, the dilution is 1:8 pleural fluid to diluent. In another embodiment, the dilution is 1:5 pleural fluid to diluent. In another embodiment, the dilution is 1:2 pleural fluid to diluent. In another embodiment, the dilution is 1:1 pleural fluid to diluent. In some embodiments, diluents include saline, phosphate buffered saline, another buffer or a physiologically acceptable diluent. In some embodiments, the sample is placed in the CellSave tube immediately after collection from the patient and dilution to avoid a decrease in the viable TILs, which may occur to a significant extent within 24-48 hours, if left in the untreated pleural fluid, even at 4° C. In some embodiments, the pleural fluid sample is placed in the appropriate collection tube within 1 hour, 5 hours, 10 hours, 15 hours, 24 hours, 36 hours, up to 48 hours after removal from the patient, and dilution. In some embodiments, the pleural fluid sample is placed in the appropriate collection tube within 1 hour, 5 hours, 10 hours, 15 hours, 24 hours, 36 hours, up to 48 hours after removal from the patient, and dilution at 4° C.

In still another embodiment, pleural fluid samples are concentrated by conventional means prior further processing steps. In some embodiments, this pre-treatment of the pleural fluid is preferable in circumstances in which the pleural fluid must be cryopreserved for shipment to a laboratory performing the method or for later analysis (e.g., later than 24-48 hours post-collection). In some embodiments, the pleural fluid sample is prepared by centrifuging the pleural fluid sample after its withdrawal from the subject and resuspending the centrifugate or pellet in buffer. In some embodiments, the pleural fluid sample is subjected to multiple centrifugations and resuspensions, before it is cryopreserved for transport or later analysis and/or processing.

In some embodiments, pleural fluid samples are concentrated prior to further processing steps by using a filtration method. In some embodiments, the pleural fluid sample used in the contacting step is prepared by filtering the fluid through a filter containing a known and essentially uniform pore size that allows for passage of the pleural fluid through the membrane but retains the tumor cells. In some embodiments, the diameter of the pores in the membrane may be at least 4 μM. In another embodiment the pore diameter may be 5 μM or more, and in other embodiment, any of 6, 7, 8, 9, or 10 μM. After filtration, the cells, including TILs, retained by the membrane may be rinsed off the membrane into a suitable physiologically acceptable buffer. Cells, including TILs, concentrated in this way may then be used in the contacting step of the method.

In some embodiments, pleural fluid sample (including, for example, the untreated pleural fluid), diluted pleural fluid, or the resuspended cell pellet, is contacted with a lytic reagent that differentially lyses non-nucleated red blood cells present in the sample. In some embodiments, this step is performed prior to further processing steps in circumstances in which the pleural fluid contains substantial numbers of RBCs. Suitable lysing reagents include a single lytic reagent or a lytic reagent and a quench reagent, or a lytic agent, a quench reagent and a fixation reagent. Suitable lytic systems are marketed commercially and include the BD Pharm Lyse™ system (Becton Dickenson). Other lytic systems include the Versalyse™ system, the FACSlyse™ system (Becton Dickenson), the Immunoprep™ system or Erythrolyse II system (Beckman Coulter, Inc.), or an ammonium chloride system. In some embodiments, the lytic reagent can vary with the primary requirements being efficient lysis of the red blood cells, and the conservation of the TILs and phenotypic properties of the TILs in the pleural fluid. In addition to employing a single reagent for lysis, the lytic systems useful in methods described herein can include a second reagent, e.g., one that quenches or retards the effect of the lytic reagent during the remaining steps of the method, e.g., Stabilyse™ reagent (Beckman Coulter, Inc.). A conventional fixation reagent may also be employed depending upon the choice of lytic reagents or the preferred implementation of the method.

In some embodiments, the pleural fluid sample, unprocessed, diluted or multiply centrifuged or processed as described herein above is cryopreserved at a temperature of about −140° C. prior to being further processed and/or expanded as provided herein.

### B. STEP B: First Expansion

In some embodiments, the present methods provide for obtaining young TILs, which are capable of increased replication cycles upon administration to a subject/patient and as such may provide additional therapeutic benefits over older TILs (i.e., TILs which have further undergone more rounds of replication prior to administration to a subject/patient). Features of young TILs have been described in the literature, for example in Donia, et al., Scand. J Immunol. 2012, 75, 157-167; Dudley, et al., Clin. Cancer Res. 2010, 16, 6122-6131; Huang, et al., J. Immunother. 2005, 28, 258-267; Besser, et al., Clin. Cancer Res. 2013, 19, OF1-OF9; Besser, et al., J. Immunother. 2009, 32, 415-423; Robbins, et al., J. Immunol. 2004, 173, 7125-7130; Shen, et al., J Immunother., 2007, 30, 123-129; Zhou, et al., J. Immunother. 2005, 28, 53-62; and Tran, et al., J Immunother., 2008, 31, 742-751, each of which is incorporated herein by reference.

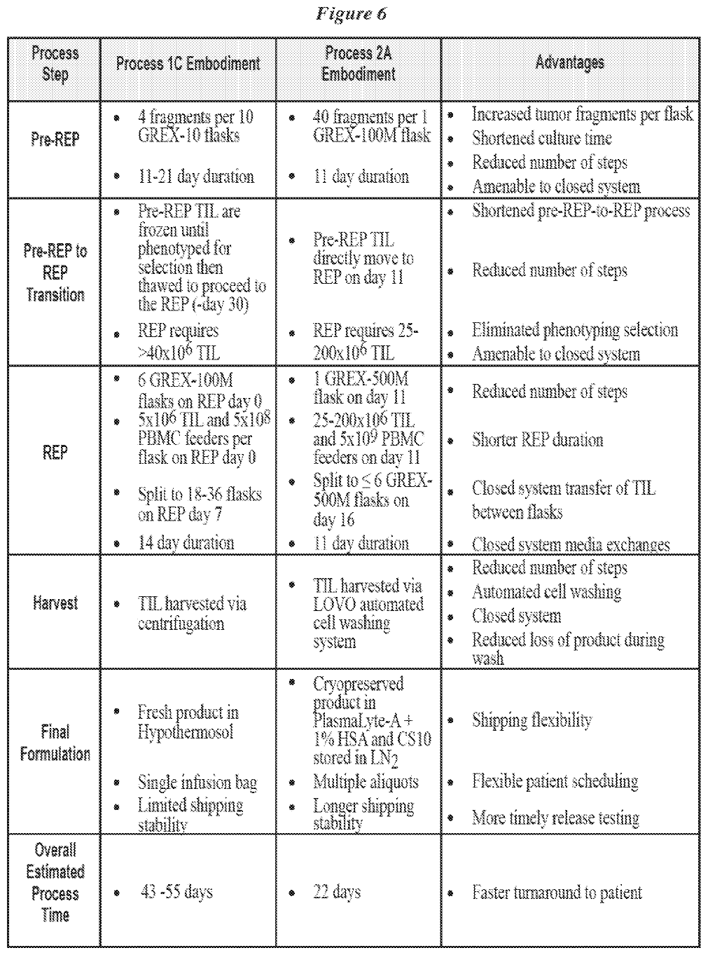

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained by the present method exhibit an increase in the T-cell repertoire diversity as compared to freshly harvested TILs and/or TILs prepared using other methods than those provide herein including for example, methods other than those embodied in FIG. 1. In some embodiments, the TILs obtained by the present method exhibit an increase in the T-cell repertoire diversity as compared to freshly harvested TILs and/or TILs prepared using methods referred to as process 1C, as exemplified in FIG. 5 and/or FIG. 6. In some embodiments, the TILs obtained in the first expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β).

After dissection or digestion of tumor fragments, for example such as described in Step A of FIG. 1, the resulting cells are cultured in serum containing IL-2 under conditions that favor the growth of TILs over tumor and other cells. In some embodiments, the tumor digests are incubated in 2 mL wells in media comprising inactivated human AB serum with 6000 IU/mL of IL-2. This primary cell population is cultured for a period of days, generally from 3 to 14 days, resulting in a bulk TIL population, generally about 1×10⁸ bulk TIL cells. In some embodiments, this primary cell population is cultured for a period of 7 to 14 days, resulting in a bulk TIL population, generally about 1×10⁸ bulk TIL cells. In some embodiments, this primary cell population is cultured for a period of 10 to 14 days, resulting in a bulk TIL population, generally about 1×10⁸ bulk TIL cells. In some embodiments, this primary cell population is cultured for a period of about 11 days, resulting in a bulk TIL population, generally about 1×10⁸ bulk TIL cells.

In a preferred embodiment, expansion of TILs may be performed using an initial bulk TIL expansion step (for example such as those described in Step B of FIG. 1, which can include processes referred to as pre-REP) as described below and herein, followed by a second expansion (Step D, including processes referred to as rapid expansion protocol (REP) steps) as described below under Step D and herein, followed by optional cryopreservation, and followed by a second Step D (including processes referred to as restimulation REP steps) as described below and herein. The TILs obtained from this process may be optionally characterized for phenotypic characteristics and metabolic parameters as described herein.

In embodiments where TIL cultures are initiated in 24-well plates, for example, using Costar 24-well cell culture cluster, flat bottom (Corning Incorporated, Corning, NY, each well can be seeded with 1×10⁶ tumor digest cells or one tumor fragment in 2 mL of complete medium (CM) with IL-2 (6000 IU/mL; Chiron Corp., Emeryville, CA). In some embodiments, the tumor fragment is between about 1 mm³ and 10 mm³.

In some embodiments, the first expansion culture medium is referred to as “CM”, an abbreviation for culture media. In some embodiments, CM for Step B consists of RPMI 1640 with GlutaMAX, supplemented with 10% human AB serum, 25 mM Hepes, and 10 mg/mL gentamicin. In embodiments where cultures are initiated in gas-permeable flasks with a 40 mL capacity and a 10 cm² gas-permeable silicon bottom (for example, G-Rex10; Wilson Wolf Manufacturing, New Brighton, MN) (FIG. 1), each flask was loaded with 10-40×10⁶ viable tumor digest cells or 5-30 tumor fragments in 10-40 mL of CM with IL-2. Both the G-Rex10 and 24-well plates were incubated in a humidified incubator at 37° C. in 5% CO₂ and 5 days after culture initiation, half the media was removed and replaced with fresh CM and IL-2 and after day 5, half the media was changed every 2-3 days.

After preparation of the tumor fragments, the resulting cells (i.e., fragments) are cultured in serum containing IL-2 under conditions that favor the growth of TILs over tumor and other cells. In some embodiments, the tumor digests are incubated in 2 mL wells in media comprising inactivated human AB serum (or, in some cases, as outlined herein, in the presence of aAPC cell population) with 6000 IU/mL of IL-2. This primary cell population is cultured for a period of days, generally from 10 to 14 days, resulting in a bulk TIL population, generally about 1×10⁸ bulk TIL cells. In some embodiments, the growth media during the first expansion comprises IL-2 or a variant thereof. In some embodiments, the IL is recombinant human IL-2 (rhIL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the IL-2 stock solution is prepare as described in Example 5. In some embodiments, the first expansion culture media comprises about 10,000 IU/mL of IL-2, about 9,000 IU/mL of IL-2, about 8,000 IU/mL of IL-2, about 7,000 IU/mL of IL-2, about 6000 IU/mL of IL-2 or about 5,000 IU/mL of IL-2. In some embodiments, the first expansion culture media comprises about 9,000 IU/mL of IL-2 to about 5,000 IU/mL of IL-2. In some embodiments, the first expansion culture media comprises about 8,000 IU/mL of IL-2 to about 6,000 IU/mL of IL-2. In some embodiments, the first expansion culture media comprises about 7,000 IU/mL of IL-2 to about 6,000 IU/mL of IL-2. In some embodiments, the first expansion culture media comprises about 6,000 IU/mL of IL-2. In an embodiment, the cell culture medium further comprises IL-2. In some embodiments, the cell culture medium comprises about 3000 IU/mL of IL-2. In an embodiment, the cell culture medium further comprises IL-2. In a preferred embodiment, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or about 8000 IU/mL of IL-2.

In some embodiments, first expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the first expansion culture media comprises about 500 IU/mL of IL-15 to about 100 IU/mL of IL-15. In some embodiments, the first expansion culture media comprises about 400 IU/mL of IL-15 to about 100 IU/mL of IL-15. In some embodiments, the first expansion culture media comprises about 300 IU/mL of IL-15 to about 100 IU/mL of IL-15. In some embodiments, the first expansion culture media comprises about 200 IU/mL of IL-15. In some embodiments, the cell culture medium comprises about 180 IU/mL of IL-15. In an embodiment, the cell culture medium further comprises IL-15. In a preferred embodiment, the cell culture medium comprises about 180 IU/mL of IL-15.

In some embodiments, first expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the first expansion culture media comprises about 20 IU/mL of IL-21 to about 0.5 IU/mL of IL-21. In some embodiments, the first expansion culture media comprises about 15 IU/mL of IL-21 to about 0.5 IU/mL of IL-21. In some embodiments, the first expansion culture media comprises about 12 IU/mL of IL-21 to about 0.5 IU/mL of IL-21. In some embodiments, the first expansion culture media comprises about 10 IU/mL of IL-21 to about 0.5 IU/mL of IL-21. In some embodiments, the first expansion culture media comprises about 5 IU/mL of IL-21 to about 1 IU/mL of IL-21. In some embodiments, the first expansion culture media comprises about 2 IU/mL of IL-21. In some embodiments, the cell culture medium comprises about 1 IU/mL of IL-21. In some embodiments, the cell culture medium comprises about 0.5 IU/mL of IL-21. In an embodiment, the cell culture medium further comprises IL-21. In a preferred embodiment, the cell culture medium comprises about 1 IU/mL of IL-21.

In an embodiment, the cell culture medium comprises OKT-3 antibody. In some embodiments, the cell culture medium comprises about 30 ng/mL of OKT-3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium does not comprise OKT-3 antibody. In some embodiments, the OKT-3 antibody is muromonab.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 pg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments, the first expansion culture medium is referred to as “CM”, an abbreviation for culture media. In some embodiments, it is referred to as CMT (culture medium 1). In some embodiments, CM consists of RPMI 1640 with GlutaMAX, supplemented with 10% human AB serum, 25 mM Hepes, and 10 mg/mL gentamicin. In embodiments where cultures are initiated in gas-permeable flasks with a 40 mL capacity and a 10 cm² gas-permeable silicon bottom (for example, G-Rex10; Wilson Wolf Manufacturing, New Brighton, MN) (FIG. 1), each flask was loaded with 10-40×10⁶ viable tumor digest cells or 5-30 tumor fragments in 10-40 mL of CM with IL-2. Both the G-Rex10 and 24-well plates were incubated in a humidified incubator at 37° C. in 5% CO₂ and 5 days after culture initiation, half the media was removed and replaced with fresh CM and IL-2 and after day 5, half the media was changed every 2-3 days. In some embodiments, the CM is the CM1 described in the Examples, see, Example 1. In some embodiments, the first expansion occurs in an initial cell culture medium or a first cell culture medium. In some embodiments, the initial cell culture medium or the first cell culture medium comprises IL-2.

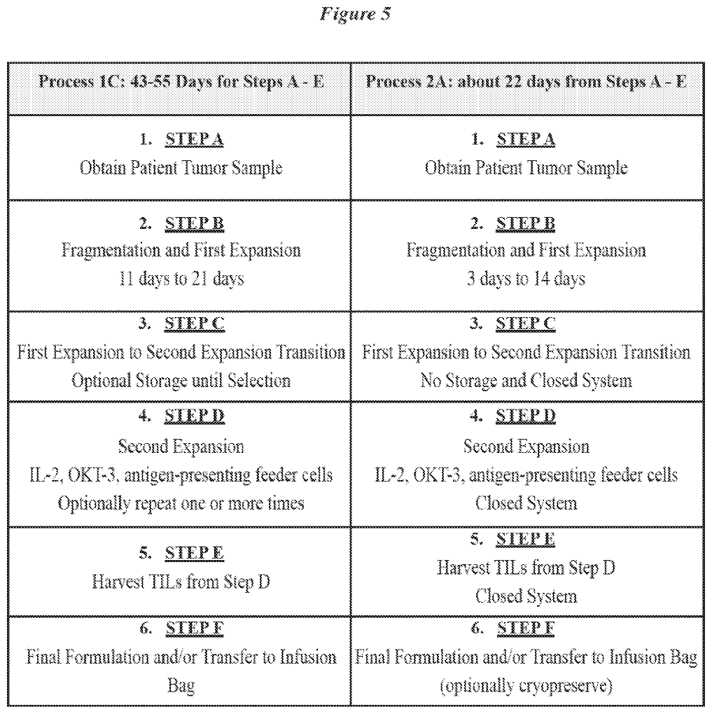

In some embodiments, the first expansion (including processes such as for example those described in Step B of FIG. 1, which can include those sometimes referred to as the pre-REP) process is shortened to 3-14 days, as discussed in the examples and figures. In some embodiments, the first expansion (including processes such as for example those described in Step B of FIG. 1, which can include those sometimes referred to as the pre-REP) is shortened to 7 to 14 days, as discussed in the Examples and shown in FIGS. 4 and 5, as well as including for example, an expansion as described in Step B of FIG. 1. In some embodiments, the first expansion of Step B is shortened to 10-14 days. In some embodiments, the first expansion is shortened to 11 days, as discussed in, for example, an expansion as described in Step B of FIG. 1.

In some embodiments, the first TIL expansion can proceed for 1 day, 2 days, 3 days, 4 days, 5 days, 6 days, 7 days, 8 days, 9 days, 10 days, 11 days, 12 days, 13 days, or 14 days. In some embodiments, the first TIL expansion can proceed for 1 day to 14 days. In some embodiments, the first TIL expansion can proceed for 2 days to 14 days. In some embodiments, the first TIL expansion can proceed for 3 days to 14 days. In some embodiments, the first TIL expansion can proceed for 4 days to 14 days. In some embodiments, the first TIL expansion can proceed for 5 days to 14 days. In some embodiments, the first TIL expansion can proceed for 6 days to 14 days. In some embodiments, the first TIL expansion can proceed for 7 days to 14 days. In some embodiments, the first TIL expansion can proceed for 8 days to 14 days. In some embodiments, the first TIL expansion can proceed for 9 days to 14 days. In some embodiments, the first TIL expansion can proceed for 10 days to 14 days. In some embodiments, the first TIL expansion can proceed for 11 days to 14 days. In some embodiments, the first TIL expansion can proceed for 12 days to 14 days. In some embodiments, the first TIL expansion can proceed for 13 days to 14 days. In some embodiments, the first TIL expansion can proceed for 14 days. In some embodiments, the first TIL expansion can proceed for 1 day to 11 days. In some embodiments, the first TIL expansion can proceed for 2 days to 11 days. In some embodiments, the first TIL expansion can proceed for 3 days to 11 days. In some embodiments, the first TIL expansion can proceed for 4 days to 11 days. In some embodiments, the first TIL expansion can proceed for 5 days to 11 days. In some embodiments, the first TIL expansion can proceed for 6 days to 11 days. In some embodiments, the first TIL expansion can proceed for 7 days to 11 days. In some embodiments, the first TIL expansion can proceed for 8 days to 11 days. In some embodiments, the first TIL expansion can proceed for 9 days to 11 days. In some embodiments, the first TIL expansion can proceed for 10 days to 11 days. In some embodiments, the first TIL expansion can proceed for 11 days.

In some embodiments, a combination of IL-2, IL-7, IL-15, and/or IL-21 are employed as a combination during the first expansion. In some embodiments, IL-2, IL-7, IL-15, and/or IL-21 as well as any combinations thereof can be included during the first expansion, including for example during a Step B processes according to FIG. 1, as well as described herein. In some embodiments, a combination of IL-2, IL-15, and IL-21 are employed as a combination during the first expansion. In some embodiments, IL-2, IL-15, and IL-21 as well as any combinations thereof can be included during Step B processes according to FIG. 1 and as described herein.

In some embodiments, the first expansion (including processes referred to as the pre-REP; for example, Step B according to FIG. 1) process is shortened to 3 to 14 days, as discussed in the examples and figures. In some embodiments, the first expansion of Step B is shortened to 7 to 14 days. In some embodiments, the first expansion of Step B is shortened to 10 to 14 days. In some embodiments, the first expansion is shortened to 11 days.

In some embodiments, the first expansion, for example, Step B according to FIG. 1, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

### 1. Cytokines and Other Additives

The expansion methods described herein generally use culture media with high doses of a cytokine, in particular IL-2, as is known in the art.

Alternatively, using combinations of cytokines for the rapid expansion and or second expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21 and IL-2, or IL-15 and IL-21, with the latter finding particular use in many embodiments. The use of combinations of cytokines specifically favors the generation of lymphocytes, and in particular T-cells as described therein.

In an embodiment, Step B may also include the addition of OKT-3 antibody or muromonab to the culture media, as described elsewhere herein. In an embodiment, Step B may also include the addition of a 4-1BB agonist to the culture media, as described elsewhere herein. In an embodiment, Step B may also include the addition of an OX-40 agonist to the culture media, as described elsewhere herein. In other embodiments, additives such as peroxisome proliferator-activated receptor gamma coactivator I-alpha agonists, including proliferator-activated receptor (PPAR)-gamma agonists such as a thiazolidinedione compound, may be used in the culture media during Step B, as described in U.S. Patent Application Publication No. US 2019/0307796 A1, the disclosure of which is incorporated by reference herein.

### C. STEP C: First Expansion to Second Expansion Transition

In some cases, the bulk TIL population obtained from the first expansion, including for example the TIL population obtained from for example, Step B as indicated in FIG. 1, can be cryopreserved immediately, using the protocols discussed herein below. Alternatively, the TIL population obtained from the first expansion, referred to as the second TIL population, can be subjected to a second expansion (which can include expansions sometimes referred to as REP) and then cryopreserved as discussed below. Similarly, in the case where genetically modified TILs will be used in therapy, the first TIL population (sometimes referred to as the bulk TIL population) or the second TIL population (which can in some embodiments include populations referred to as the REP TIL populations) can be subjected to genetic modifications for suitable treatments prior to expansion or after the first expansion and prior to the second expansion.

In some embodiments, the TILs obtained from the first expansion (for example, from Step B as indicated in FIG. 1) are stored until phenotyped for selection. In some embodiments, the TILs obtained from the first expansion (for example, from Step B as indicated in FIG. 1) are not stored and proceed directly to the second expansion. In some embodiments, the TILs obtained from the first expansion are not cryopreserved after the first expansion and prior to the second expansion. In some embodiments, the transition from the first expansion to the second expansion occurs at about 3 days, 4, days, 5 days, 6 days, 7 days, 8 days, 9 days, 10 days, 11 days, 12 days, 13 days, or 14 days from when fragmentation occurs. In some embodiments, the transition from the first expansion to the second expansion occurs at about 3 days to 14 days from when fragmentation occurs. In some embodiments, the transition from the first expansion to the second expansion occurs at about 4 days to 14 days from when fragmentation occurs. In some embodiments, the transition from the first expansion to the second expansion occurs at about 4 days to 10 days from when fragmentation occurs. In some embodiments, the transition from the first expansion to the second expansion occurs at about 7 days to 14 days from when fragmentation occurs. In some embodiments, the transition from the first expansion to the second expansion occurs at about 14 days from when fragmentation occurs.

In some embodiments, the transition from the first expansion to the second expansion occurs at 1 day, 2 days, 3 days, 4 days, 5 days, 6 days, 7 days, 8 days, 9 days, 10 days, 11 days, 12 days, 13 days, or 14 days from when fragmentation occurs. In some embodiments, the transition from the first expansion to the second expansion occurs 1 day to 14 days from when fragmentation occurs. In some embodiments, the first TIL expansion can proceed for 2 days to 14 days. In some embodiments, the transition from the first expansion to the second expansion occurs 3 days to 14 days from when fragmentation occurs. In some embodiments, the transition from the first expansion to the second expansion occurs 4 days to 14 days from when fragmentation occurs. In some embodiments, the transition from the first expansion to the second expansion occurs 5 days to 14 days from when fragmentation occurs. In some embodiments, the transition from the first expansion to the second expansion occurs 6 days to 14 days from when fragmentation occurs. In some embodiments, the transition from the first expansion to the second expansion occurs 7 days to 14 days from when fragmentation occurs. In some embodiments, the transition from the first expansion to the second expansion occurs 8 days to 14 days from when fragmentation occurs. In some embodiments, the transition from the first expansion to the second expansion occurs 9 days to 14 days from when fragmentation occurs. In some embodiments, the transition from the first expansion to the second expansion occurs 10 days to 14 days from when fragmentation occurs. In some embodiments, the transition from the first expansion to the second expansion occurs 11 days to 14 days from when fragmentation occurs. In some embodiments, the transition from the first expansion to the second expansion occurs 12 days to 14 days from when fragmentation occurs. In some embodiments, the transition from the first expansion to the second expansion occurs 13 days to 14 days from when fragmentation occurs. In some embodiments, the transition from the first expansion to the second expansion occurs 14 days from when fragmentation occurs. In some embodiments, the transition from the first expansion to the second expansion occurs 1 day to 11 days from when fragmentation occurs. In some embodiments, the transition from the first expansion to the second expansion occurs 2 days to 11 days from when fragmentation occurs. In some embodiments, the transition from the first expansion to the second expansion occurs 3 days to 11 days from when fragmentation occurs. In some embodiments, the transition from the first expansion to the second expansion occurs 4 days to 11 days from when fragmentation occurs. In some embodiments, the transition from the first expansion to the second expansion occurs 5 days to 11 days from when fragmentation occurs. In some embodiments, the transition from the first expansion to the second expansion occurs 6 days to 11 days from when fragmentation occurs. In some embodiments, the transition from the first expansion to the second expansion occurs 7 days to 11 days from when fragmentation occurs. In some embodiments, the transition from the first expansion to the second expansion occurs 8 days to 11 days from when fragmentation occurs. In some embodiments, the transition from the first expansion to the second expansion occurs 9 days to 11 days from when fragmentation occurs. In some embodiments, the transition from the first expansion to the second expansion occurs 10 days to 11 days from when fragmentation occurs. In some embodiments, the transition from the first expansion to the second expansion occurs 11 days from when fragmentation occurs.

In some embodiments, the TILs are not stored after the first expansion and prior to the second expansion, and the TILs proceed directly to the second expansion (for example, in some embodiments, there is no storage during the transition from Step B to Step D as shown in FIG. 1). In some embodiments, the transition occurs in closed system, as described herein. In some embodiments, the TILs from the first expansion, the second population of TILs, proceeds directly into the second expansion with no transition period.

In some embodiments, the transition from the first expansion to the second expansion, for example, Step C according to FIG. 1, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100 bioreactor. In some embodiments, the closed system bioreactor is a single bioreactor.

### D. STEP D: Second Expansion

In some embodiments, the TIL cell population is expanded in number after harvest and initial bulk processing for example, after Step A and Step B, and the transition referred to as Step C, as indicated in FIG. 1). This further expansion is referred to herein as the second expansion, which can include expansion processes generally referred to in the art as a rapid expansion process (REP); as well as processes as indicated in Step D of FIG. 1. The second expansion is generally accomplished using a culture media comprising a number of components, including feeder cells, a cytokine source, and an anti-CD3 antibody, in a gas-permeable container.

In some embodiments, the second expansion or second TIL expansion (which can include expansions sometimes referred to as REP; as well as processes as indicated in Step D of FIG. 1) of TIL can be performed using any TIL flasks or containers known by those of skill in the art. In some embodiments, the second TIL expansion can proceed for 7 days, 8 days, 9 days, 10 days, 11 days, 12 days, 13 days, or 14 days. In some embodiments, the second TIL expansion can proceed for about 7 days to about 14 days. In some embodiments, the second TIL expansion can proceed for about 8 days to about 14 days. In some embodiments, the second TIL expansion can proceed for about 9 days to about 14 days. In some embodiments, the second TIL expansion can proceed for about 10 days to about 14 days. In some embodiments, the second TIL expansion can proceed for about 11 days to about 14 days. In some embodiments, the second TIL expansion can proceed for about 12 days to about 14 days. In some embodiments, the second TIL expansion can proceed for about 13 days to about 14 days. In some embodiments, the second TIL expansion can proceed for about 14 days.

In an embodiment, the second expansion can be performed in a gas permeable container using the methods of the present disclosure (including for example, expansions referred to as REP; as well as processes as indicated in Step D of FIG. 1). For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/mL of OKT3, a mouse monoclonal anti-CD3 antibody (commercially available from Ortho-McNeil, Raritan, NJ or Miltenyi Biotech, Auburn, CA) or UHCT-1 (commercially available from BioLegend, San Diego, CA, USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In an embodiment, the cell culture medium further comprises IL-2. In some embodiments, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In an embodiment, the cell culture medium comprises OKT-3 antibody. In some embodiments, the cell culture medium comprises about 30 ng/mL of OKT-3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 pg/mL of OKT-3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium does not comprise OKT-3 antibody. In some embodiments, the OKT-3 antibody is muromonab.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 pg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments, a combination of IL-2, IL-7, IL-15, and/or IL-21 are employed as a combination during the second expansion. In some embodiments, IL-2, IL-7, IL-15, and/or IL-21 as well as any combinations thereof can be included during the second expansion, including for example during a Step D processes according to FIG. 1, as well as described herein. In some embodiments, a combination of IL-2, IL-15, and IL-21 are employed as a combination during the second expansion. In some embodiments, IL-2, IL-15, and IL-21 as well as any combinations thereof can be included during Step D processes according to FIG. 1 and as described herein.

In some embodiments, the second expansion can be conducted in a supplemented cell culture medium comprising IL-2, OKT-3, antigen-presenting feeder cells, and optionally a TNFRSF agonist. In some embodiments, the second expansion occurs in a supplemented cell culture medium. In some embodiments, the supplemented cell culture medium comprises IL-2, OKT-3, and antigen-presenting feeder cells. In some embodiments, the second cell culture medium comprises IL-2, OKT-3, and antigen-presenting cells (APCs; also referred to as antigen-presenting feeder cells). In some embodiments, the second expansion occurs in a cell culture medium comprising IL-2, OKT-3, and antigen-presenting feeder cells (i.e., antigen presenting cells).

In some embodiments, the second expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the second expansion culture media comprises about 500 IU/mL of IL-15 to about 100 IU/mL of IL-15. In some embodiments, the second expansion culture media comprises about 400 IU/mL of IL-15 to about 100 IU/mL of IL-15. In some embodiments, the second expansion culture media comprises about 300 IU/mL of LL-15 to about 100 IU/mL of IL-15. In some embodiments, the second expansion culture media comprises about 200 IU/mL of IL-15. In some embodiments, the cell culture medium comprises about 180 IU/mL of IL-15. In an embodiment, the cell culture medium further comprises IL-15. In a preferred embodiment, the cell culture medium comprises about 180 IU/mL of IL-15.

In some embodiments, the second expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the second expansion culture media comprises about 20 IU/mL of IL-21 to about 0.5 IU/mL of IL-21. In some embodiments, the second expansion culture media comprises about 15 IU/mL of IL-21 to about 0.5 IU/mL of IL-21. In some embodiments, the second expansion culture media comprises about 12 IU/mL of IL-21 to about 0.5 IU/mL of IL-21. In some embodiments, the second expansion culture media comprises about 10 IU/mL of IL-21 to about 0.5 IU/mL of IL-21. In some embodiments, the second expansion culture media comprises about 5 IU/mL of IL-21 to about 1 IU/mL of IL-21. In some embodiments, the second expansion culture media comprises about 2 IU/mL of IL-21. In some embodiments, the cell culture medium comprises about 1 IU/mL of IL-21. In some embodiments, the cell culture medium comprises about 0.5 IU/mL of IL-21. In an embodiment, the cell culture medium further comprises IL-21. In a preferred embodiment, the cell culture medium comprises about 1 IU/mL of IL-21.

In some embodiments the antigen-presenting feeder cells (APCs) are PBMCs. In an embodiment, the ratio of TILs to PBMCs and/or antigen-presenting cells in the rapid expansion and/or the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to PBMCs in the rapid expansion and/or the second expansion is between 1 to 50 and 1 to 300. In an embodiment, the ratio of TILs to PBMCs in the rapid expansion and/or the second expansion is between 1 to 100 and 1 to 200.

In an embodiment, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 mL media. Media replacement is done (generally ⅔ media replacement via respiration with fresh media) until the cells are transferred to an alternative growth chamber. Alternative growth chambers include G-REX flasks and gas permeable containers as more fully discussed below.

In some embodiments, the second expansion (which can include processes referred to as the REP process) is shortened to 7-14 days, as discussed in the examples and figures. In some embodiments, the second expansion is shortened to 11 days.

In an embodiment, REP and/or the second expansion may be performed using T-175 flasks and gas permeable bags as previously described (Tran, et al., J. Immunother. 2008, 31, 742-51; Dudley, et al., J. Immunother. 2003, 26, 332-42) or gas permeable cultureware (G-Rex flasks). In some embodiments, the second expansion (including expansions referred to as rapid expansions) is performed in T-175 flasks, and about 1×10⁶ TILs suspended in 150 mL of media may be added to each T-175 flask. The TILs may be cultured in a 1 to 1 mixture of CM and AIM-V medium, supplemented with 3000 IU per mL of IL-2 and 30 ng per mL of anti-CD3. The T-175 flasks may be incubated at 37° C. in 5% CO₂. Half the media may be exchanged on day 5 using 50/50 medium with 3000 IU per mL of IL-2. In some embodiments, on day 7 cells from two T-175 flasks may be combined in a 3 L bag and 300 mL of AIM V with 5% human AB serum and 3000 IU per mL of IL-2 was added to the 300 mL of TIL suspension. The number of cells in each bag was counted every day or two and fresh media was added to keep the cell count between 0.5 and 2.0×10⁶ cells/mL.

In an embodiment, the second expansion (which can include expansions referred to as REP, as well as those referred to in Step D of FIG. 1) may be performed in 500 mL capacity gas permeable flasks with 100 cm gas-permeable silicon bottoms (G-Rex 100, commercially available from Wilson Wolf Manufacturing Corporation, New Brighton, MN, USA), 5×10⁶ or 10×10⁶ TIL may be cultured with PBMCs in 400 mL of 50/50 medium, supplemented with 5% human AB serum, 3000 IU per mL of IL-2 and 30 ng per mL of anti-CD3 (OKT3). The G-Rex 100 flasks may be incubated at 37° C. in 5% CO₂. On day 5, 250 mL of supernatant may be removed and placed into centrifuge bottles and centrifuged at 1500 rpm (491×g) for 10 minutes. The TIL pellets may be re-suspended with 150 mL of fresh medium with 5% human AB serum, 3000 IU per mL of IL-2, and added back to the original G-Rex 100 flasks. When TILs are expanded serially in G-Rex 100 flasks, on day 7 the TIL in each G-Rex 100 may be suspended in the 300 mL of media present in each flask and the cell suspension may be divided into 3 100 mL aliquots that may be used to seed 3 G-Rex 100 flasks. Then 150 mL of AIM-V with 5% human AB serum and 3000 IU per mL of IL-2 may be added to each flask. The G-Rex 100 flasks may be incubated at 37° C. in 5% CO₂ and after 4 days 150 mL of AIM-V with 3000 IU per mL of IL-2 may be added to each G-REX 100 flask. The cells may be harvested on day 14 of culture.

In an embodiment, the second expansion (including expansions referred to as REP) is performed in flasks with the bulk TILs being mixed with a 100- or 200-fold excess of inactivated feeder cells, 30 mg/mL OKT3 anti-CD3 antibody and 3000 IU/mL IL-2 in 150 mL media. In some embodiments, media replacement is done until the cells are transferred to an alternative growth chamber. In some embodiments, ⅔ of the media is replaced by respiration with fresh media. In some embodiments, alternative growth chambers include G-REX flasks and gas permeable containers as more fully discussed below.

In an embodiment, the second expansion (including expansions referred to as REP) is performed and further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

Optionally, a cell viability assay can be performed after the second expansion (including expansions referred to as the REP expansion), using standard assays known in the art. For example, a try pan blue exclusion assay can be done on a sample of the bulk TILs, which selectively labels dead cells and allows a viability assessment. In some embodiments, TIL samples can be counted and viability determined using a Cellometer K2 automated cell counter (Nexcelom Bioscience, Lawrence, MA). In some embodiments, viability is determined according to the standard Cellometer K2 Image Cytometer Automatic Cell Counter protocol.

In some embodiments, the second expansion (including expansions referred to as REP) of TIL can be performed using T-175 flasks and gas-permeable bags as previously described (Tran, et al., 2008, J Immunother., 31, 742-751, and Dudley, et al. 2003, J Immunother., 26, 332-342) or gas-permeable G-Rex flasks. In some embodiments, the second expansion is performed using flasks. In some embodiments, the second expansion is performed using gas-permeable G-Rex flasks. In some embodiments, the second expansion is performed in T-175 flasks, and about 1×10⁶ TILs are suspended in about 150 mL of media and this is added to each T-175 flask. The TILs are cultured with irradiated (50 Gy) allogeneic PBMC as “feeder” cells at a ratio of 1 to 100 and the cells were cultured in a 1 to 1 mixture of CM and AIM-V medium (50/50 medium), supplemented with 3000 IU/mL of IL-2 and 30 ng/mL of anti-CD3. The T-175 flasks are incubated at 37° C. in 5% CO₂. In some embodiments, half the media is changed on day 5 using 50/50 medium with 3000 IU/mL of IL-2. In some embodiments, on day 7, cells from 2 T-175 flasks are combined in a 3 L bag and 300 mL of AIM-V with 5% human AB serum and 3000 IU/mL of IL-2 is added to the 300 mL of TIL suspension. The number of cells in each bag can be counted every day or two and fresh media can be added to keep the cell count between about 0.5 and about 2.0×10⁶ cells/mL.

In some embodiments, the second expansion (including expansions referred to as REP) are performed in 500 mL capacity flasks with 100 cm² gas-permeable silicon bottoms (G-Rex 100, Wilson Wolf) (FIG. 1), about 5×10⁶ or 10×10⁶ TILs are cultured with irradiated allogeneic PBMC at a ratio of 1 to 100 in 400 mL of 50/50 medium, supplemented with 3000 IU/mL of IL-2 and 30 ng/mL of anti-CD3. The G-Rex 100 flasks are incubated at 37° C. in 5% CO₂. In some embodiments, on day 5, 250 mL of supernatant is removed and placed into centrifuge bottles and centrifuged at 1500 rpm (491 g) for 10 minutes. The TIL pellets can then be resuspended with 150 mL of fresh 50/50 medium with 3000 IU/mL of IL-2 and added back to the original G-Rex 100 flasks. In embodiments where TILs are expanded serially in G-Rex 100 flasks, on day 7 the TIL in each G-Rex 100 are suspended in the 300 mL of media present in each flask and the cell suspension was divided into three 100 mL aliquots that are used to seed 3 G-Rex 100 flasks. Then 150 mL of AIM-V with 5% human AB serum and 3000 IU/mL of IL-2 is added to each flask. The G-Rex 100 flasks are incubated at 37° C. in 5% CO₂ and after 4 days 150 mL of AIM-V with 3000 IU/mL of IL-2 is added to each G-Rex 100 flask. The cells are harvested on day 14 of culture.

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained in the second expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRCα/β).

In some embodiments, the second expansion culture medium (e.g., sometimes referred to as CM2 or the second cell culture medium), comprises IL-2, OKT-3, as well as the antigen-presenting feeder cells (APCs), as discussed in more detail below.

In some embodiments, the second expansion, for example, Step D according to FIG. 1, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

### 1. Feeder Cells and Antigen Presenting Cells

In an embodiment, the second expansion procedures described herein (for example including expansion such as those described in Step D from FIG. 1, as well as those referred to as REP) require an excess of feeder cells during REP TIL expansion and/or during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogeneic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and accepted for use in the TIL expansion procedures described herein if the total number of viable cells on day 14 is less than the initial viable cell number put into culture on day 0 of the REP and/or day 0 of the second expansion (i.e., the start day of the second expansion).

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/mL OKT3 antibody and 3000 IU/mL IL-2.

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 5-60 ng/mL OKT3 antibody and 1000-6000 IU/mL IL-2. In some embodiments, the PBMCs are cultured in the presence of 10-50 ng/mL OKT3 antibody and 2000-5000 IU/mL IL-2. In some embodiments, the PBMCs are cultured in the presence of 20-40 ng/mL OKT3 antibody and 2000-4000 IU/mL IL-2. In some embodiments, the PBMCs are cultured in the presence of 25-35 ng/mL OKT3 antibody and 2500-3500 IU/mL IL-2.

In some embodiments, the antigen-presenting feeder cells are PBMCs. In some embodiments, the antigen-presenting feeder cells are artificial antigen-presenting feeder cells. In an embodiment, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to antigen-presenting feeder cells in the second expansion is between 1 to 50 and 1 to 300. In an embodiment, the ratio of TILs to antigen-presenting feeder cells in the second expansion is between 1 to 100 and 1 to 200.

In an embodiment, the second expansion procedures described herein require a ratio of about 2.5×10⁹ feeder cells to about 100×10⁶ TILs. In another embodiment, the second expansion procedures described herein require a ratio of about 2.5×10⁹ feeder cells to about 50×10⁶ TILs. In yet another embodiment, the second expansion procedures described herein require about 2.5×10⁹ feeder cells to about 25×10⁶ TILs.

In an embodiment, the second expansion procedures described herein require an excess of feeder cells during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation. In an embodiment, artificial antigen-presenting (aAPC) cells are used in place of PBMCs.

In general, the allogeneic PBMCs are inactivated, either via irradiation or heat treatment, and used in the TIL expansion procedures described herein, including the exemplary procedures described in the figures and examples.

In an embodiment, artificial antigen presenting cells are used in the second expansion as a replacement for, or in combination with, PBMCs.

### 2. Cytokines and Other Additives

The expansion methods described herein generally use culture media with high doses of a cytokine, in particular IL-2, as is known in the art.

Alternatively, using combinations of cytokines for the rapid expansion and or second expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21 and IL-2, IL-15 and IL-21, with the latter finding particular use in many embodiments. The use of combinations of cytokines specifically favors the generation of lymphocytes, and in particular T-cells as described therein.

In an embodiment, Step D may also include the addition of OKT-3 antibody or muromonab to the culture media, as described elsewhere herein. In an embodiment, Step D may also include the addition of a 4-1BB agonist to the culture media, as described elsewhere herein. In an embodiment, Step D may also include the addition of an OX-40 agonist to the culture media, as described elsewhere herein. In addition, additives such as peroxisome proliferator-activated receptor gamma coactivator I-alpha agonists, including proliferator-activated receptor (PPAR)-gamma agonists such as a thiazolidinedione compound, may be used in the culture media during Step D, as described in U.S. Patent Application Publication No. US 2019/0307796 A1, the disclosure of which is incorporated by reference herein.

### E. STEP E: Harvest TILs

After the second expansion step, cells can be harvested. In some embodiments the TILs are harvested after one, two, three, four or more expansion steps, for example as provided in FIG. 1. In some embodiments the TILs are harvested after two expansion steps, for example as provided in FIG. 1.

TILs can be harvested in any appropriate and sterile manner, including for example by centrifugation. Methods for TIL harvesting are well known in the art and any such know methods can be employed with the present process. In some embodiments, TILs are harvest using an automated system.

Cell harvesters and/or cell processing systems are commercially available from a variety of sources, including, for example, Fresenius Kabi, Tomtec Life Science, Perkin Elmer, and Inotech Biosystems International, Inc. Any cell based harvester can be employed with the present methods. In some embodiments, the cell harvester and/or cell processing systems is a membrane-based cell harvester. In some embodiments, cell harvesting is via a cell processing system, such as the LOVO system (manufactured by Fresenius Kabi). The term “LOVO cell processing system” also refers to any instrument or device manufactured by any vendor that can pump a solution comprising cells through a membrane or filter such as a spinning membrane or spinning filter in a sterile and/or closed system environment, allowing for continuous flow and cell processing to remove supernatant or cell culture media without pelletization. In some embodiments, the cell harvester and/or cell processing system can perform cell separation, washing, fluid-exchange, concentration, and/or other cell processing steps in a closed, sterile system.

In some embodiments, the harvest, for example, Step E according to FIG. 1, is performed from a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

In some embodiments, Step E according to FIG. 1, is performed according to the processes described herein. In some embodiments, the closed system is accessed via syringes under sterile conditions in order to maintain the sterility and closed nature of the system. In some embodiments, a closed system as described in the Examples is employed.

In some embodiments, TILs are harvested according to the methods described in the Examples. In some embodiments, TILs between days 1 and 11 are harvested using the methods as described in the steps referred herein, such as in the day 11 TIL harvest in the Examples. In some embodiments, TILs between days 12 and 22 are harvested using the methods as described in the steps referred herein, such as in the Day 22 TIL harvest in the Examples.

### F. STEP F: Final Formulation and Transfer to Infusion Container

After Steps A through E as provided in an exemplary order in FIG. 1 and as outlined in detailed above and herein are complete, cells are transferred to a container for use in administration to a patient, such as an infusion bag or sterile vial. In some embodiments, once a therapeutically sufficient number of TILs are obtained using the expansion methods described above, they are transferred to a container for use in administration to a patient.

In an embodiment, TILs expanded using APCs of the present disclosure are administered to a patient as a pharmaceutical composition. In an embodiment, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

### IV. Gen 3 TIL Manufacturing Processes

Without being limited to any particular theory, it is believed that the priming first expansion that primes an activation of T cells followed by the rapid second expansion that boosts the activation of T cells as described in the methods of the invention allows the preparation of expanded T cells that retain a “younger” phenotype, and as such the expanded T cells of the invention are expected to exhibit greater cytotoxicity against cancer cells than T cells expanded by other methods. In particular, it is believed that an activation of T cells that is primed by exposure to an anti-CD3 antibody (e.g. OKT-3), IL-2 and optionally antigen-presenting cells (APCs) and then boosted by subsequent exposure to additional anti-CD-3 antibody (e.g. OKT-3), IL-2 and APCs as taught by the methods of the invention limits or avoids the maturation of T cells in culture, yielding a population of T cells with a less mature phenotype, which T cells are less exhausted by expansion in culture and exhibit greater cytotoxicity against cancer cells. In some embodiments, the step of rapid second expansion is split into a plurality of steps to achieve a scaling up of the culture by: (a) performing the rapid second expansion by culturing T cells in a small scale culture in a first container, e.g., a G-REX 100 MCS container, for a period of about 3 to 4 days, and then (b) effecting the transfer of the T cells in the small scale culture to a second container larger than the first container, e.g., a G-REX 500 MCS container, and culturing the T cells from the small scale culture in a larger scale culture in the second container for a period of about 4 to 7 days. In some embodiments, the step of rapid expansion is split into a plurality of steps to achieve a scaling out of the culture by: (a) performing the rapid second expansion by culturing T cells in a first small scale culture in a first container, e.g., a G-REX 100 MCS container, for a period of about 3 to 4 days, and then (b) effecting the transfer and apportioning of the T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the T cells from first small scale culture transferred to such second container is cultured in a second small scale culture for a period of about 4 to 7 days. In some embodiments, the step of rapid expansion is split into a plurality of steps to achieve a scaling out and scaling up of the culture by: (a) performing the rapid second expansion by culturing T cells in a small scale culture in a first container, e.g., a G-REX 100 MCS container, for a period of about 3 to 4 days, and then (b) effecting the transfer and apportioning of the T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the T cells from the small scale culture transferred to such second container is cultured in a larger scale culture for a period of about 4 to 7 days. In some embodiments, the step of rapid expansion is split into a plurality of steps to achieve a scaling out and scaling up of the culture by: (a) performing the rapid second expansion by culturing T cells in a small scale culture in a first container, e.g., a G-REX 100 MCS container, for a period of about 4 days, and then (b) effecting the transfer and apportioning of the T cells from the small scale culture into and amongst 2, 3 or 4 second containers that are larger in size than the first container, e.g., G-REX 500 MCS containers, wherein in each second container the portion of the T cells from the small scale culture transferred to such second container is cultured in a larger scale culture for a period of about 5 days.

In some embodiments, the rapid second expansion is performed after the activation of T cells effected by the priming first expansion begins to decrease, abate, decay or subside.

In some embodiments, the rapid second expansion is performed after the activation of T cells effected by the priming first expansion has decreased by at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

In some embodiments, the rapid second expansion is performed after the activation of T cells effected by the priming first expansion has decreased by a percentage in the range of at or about 1% to 100%.

In some embodiments, the rapid second expansion is performed after the activation of T cells effected by the priming first expansion has decreased by a percentage in the range of at or about 1% to 10%, 10% to 20%, 20% to 30%, 30% to 40%, 40% to 50%, 50% to 60%, 60% to 70%, 70% to 80%, 80% to 90%, or 90% to 100%.

In some embodiments, the rapid second expansion is performed after the activation of T cells effected by the priming first expansion has decreased by at least at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

In some embodiments, the rapid second expansion is performed after the activation of T cells effected by the priming first expansion has decreased by up to at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In some embodiments, the decrease in the activation of T cells effected by the priming first expansion is determined by a reduction in the amount of interferon gamma released by the T cells in response to stimulation with antigen.

In some embodiments, the priming first expansion of T cells is performed during a period of up to at or about 7 days or about 8 days.

In some embodiments, the priming first expansion of T cells is performed during a period of up to at or about 1 day, 2 days, 3 days, 4 days, 5 days. 6 days, 7 days, or 8 days.

In some embodiments, the priming first expansion of T cells is performed during a period of 1 day, 2 days, 3 days, 4 days, 5 days, 6 days, 7 days, or 8 days.

In some embodiments, the rapid second expansion of T cells is performed during a period of up to at or about 11 days.

In some embodiments, the rapid second expansion of T cells is performed during a period of up to at or about 1 day, 2 days, 3 days, 4 days, 5 days. 6 days, 7 days, 8 days, 9 days, 10 days or 11 days.

In some embodiments, the rapid second expansion of T cells is performed during a period of 1 day, 2 days, 3 days, 4 days, 5 days, 6 days, 7 days, 8 days, 9 days, 10 days or 11 days.

In some embodiments, the priming first expansion of T cells is performed during a period of from at or about 1 day to at or about 7 days and the rapid second expansion of T cells is performed during a period of from at or about 1 day to at or about 11 days.

In some embodiments, the priming first expansion of T cells is performed during a period of up to at or about 1 day, 2 days, 3 days, 4 days, 5 days, 6 days, 7 days, or 8 days and the rapid second expansion of T cells is performed during a period of up to at or about 1 day, 2 days, 3 days, 4 days, 5 days, 6 days, 7 days, 8 days, 9 days, 10 days or 11 days.

In some embodiments, the priming first expansion of T cells is performed during a period of from at or about 1 day to at or about 8 days and the rapid second expansion of T cells is performed during a period of from at or about 1 day to at or about 9 days.

In some embodiments, the priming first expansion of T cells is performed during a period of 8 days and the rapid second expansion of T cells is performed during a period of 9 days.

In some embodiments, the priming first expansion of T cells is performed during a period of from at or about 1 day to at or about 7 days and the rapid second expansion of T cells is performed during a period of from at or about 1 day to at or about 9 days.

In some embodiments, the priming first expansion of T cells is performed during a period of 7 days and the rapid second expansion of T cells is performed during a period of 9 days.

In some embodiments, the T cells are tumor infiltrating lymphocytes (TILs).

In some embodiments, the T cells are marrow infiltrating lymphocytes (MILs).

In some embodiments, the T cells are peripheral blood lymphocytes (PBLs).

In some embodiments, the T cells are obtained from a donor suffering from a cancer.

In some embodiments, the T cells are TILs obtained from a tumor excised from a patient suffering from a cancer.

In some embodiments, the T cells are MILs obtained from bone marrow of a patient suffering from a hematologic malignancy.

In some embodiments, the T cells are PBLs obtained from peripheral blood mononuclear cells (PBMCs) from a donor. In some embodiments, the donor is suffering from a cancer. In some embodiments, the cancer is the cancer is selected from the group consisting of melanoma, ovarian cancer, endometrial cancer, thyroid cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the donor is suffering from a tumor. In some embodiments, the tumor is a liquid tumor. In some embodiments, the tumor is a solid tumor. In some embodiments, the donor is suffering from a hematologic malignancy.

In certain aspects of the present disclosure, immune effector cells, e.g., T cells, can be obtained from a unit of blood collected from a subject using any number of techniques known to the skilled artisan, such as FICOLL separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. In one aspect, T cells are isolated from peripheral blood lymphocytes by lysing the red blood cells and depleting the monocytes, for example, by centrifugation through a PERCOLL gradient or by counterflow centrifugal elutriation.

In some embodiments, the T cells are PBLs separated from whole blood or apheresis product enriched for lymphocytes from a donor. In some embodiments, the donor is suffering from a cancer. In some embodiments, the cancer is the cancer is selected from the group consisting of melanoma, ovarian cancer, endometrial cancer, thyroid cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the donor is suffering from a tumor. In some embodiments, the tumor is a liquid tumor. In some embodiments, the tumor is a solid tumor. In some embodiments, the donor is suffering from a hematologic malignancy. In some embodiments, the PBLs are isolated from whole blood or apheresis product enriched for lymphocytes by using positive or negative selection methods, i.e., removing the PBLs using a marker(s), e.g., CD3+ CD45+, for T cell phenotype, or removing non-T cell phenotype cells, leaving PBLs. In other embodiments, the PBLs are isolated by gradient centrifugation. Upon isolation of PBLs from donor tissue, the priming first expansion of PBLs can be initiated by seeding a suitable number of isolated PBLs (in some embodiments, approximately 1×10⁷ PBLs) in the priming first expansion culture according to the priming first expansion step of any of the methods described herein.

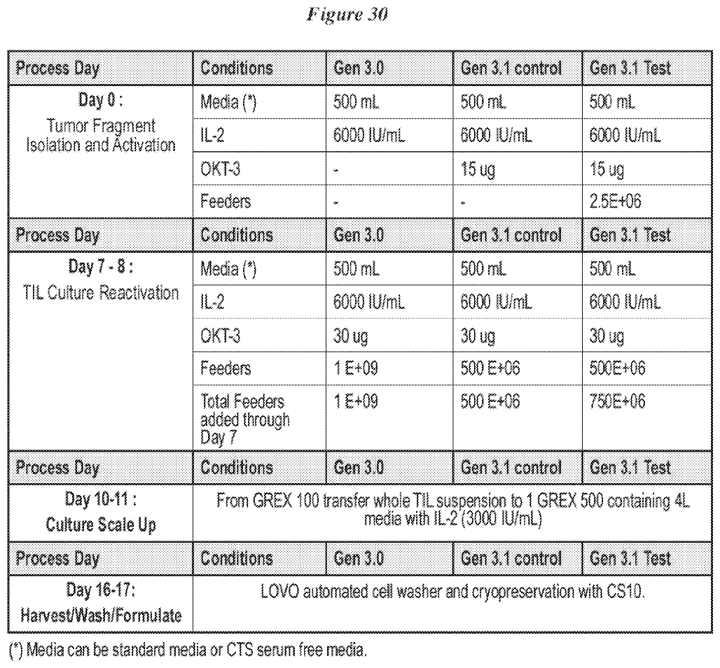

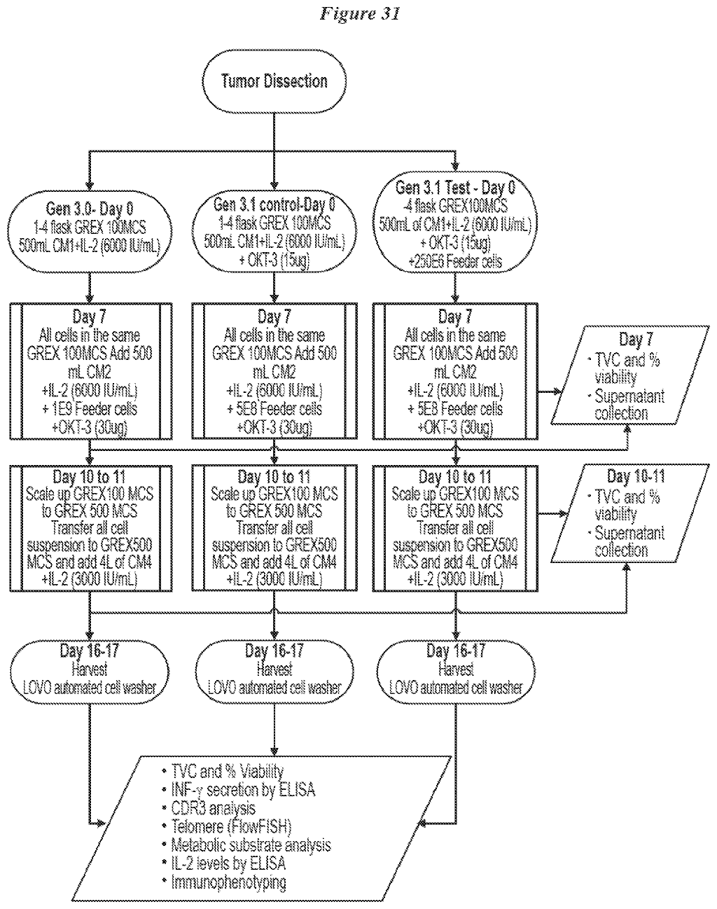

An exemplary TIL process known as process 3 (also referred to herein as GEN 3) containing some of these features is depicted in FIG. 8 (in particular, e.g., FIG. 8B and/or FIG. 8C), and some of the advantages of this embodiment of the present invention over Gen 2 are described in FIGS. 1, 2, 30, and 31 (in particular, e.g., FIG. 8B and/or FIG. 8C). Two embodiments of process 3 are shown in FIGS. 1 and 30 (in particular, e.g., FIG. 8B and/or FIG. 8C). Gen 2 or Gen 2A is also described in U.S. Patent Publication No. 2018/0280436, incorporated by reference herein in its entirety. The Gen 3 process is also described in U.S. Ser. No. 62/755,954 filed on Nov. 5, 2018 (116983-5045-PR).

As discussed and generally outlined herein, TILs are taken from a patient sample and manipulated to expand their number prior to transplant into a patient using the TIL expansion process described herein and referred to as Gen 3. In some embodiments, the TILs may be optionally genetically manipulated as discussed below. In some embodiments, the TILs may be cryopreserved prior to or after expansion. Once thawed, they may also be restimulated to increase their metabolism prior to infusion into a patient.

In some embodiments, the priming first expansion (including processes referred herein as the pre-Rapid Expansion (Pre-REP), as well as processes shown in FIG. 8 (in particular, e.g., FIG. 8B and/or FIG. 8C) as Step B) is shortened to 1 to 8 days and the rapid second expansion (including processes referred to herein as Rapid Expansion Protocol (REP) as well as processes shown in FIG. 1 (in particular, e.g., FIG. 8B and/or FIG. 8C) as Step D) is shortened to 1 to 9 days, as discussed in detail below as well as in the examples and figures. In some embodiments, the priming first expansion (including processes referred herein as the pre-Rapid Expansion (Pre-REP), as well as processes shown in FIG. 1 (in particular, e.g., FIG. 8B and/or FIG. 8C) as Step B) is shortened to 1 to 8 days and the rapid second expansion (including processes referred to herein as Rapid Expansion Protocol (REP) as well as processes shown in FIG. 1 (in particular, e.g., FIG. 8B and/or FIG. 8C) as Step D) is shortened to 1 to 8 days, as discussed in detail below as well as in the examples and figures. In some embodiments, the priming first expansion (including processes referred herein as the pre-Rapid Expansion (Pre-REP), as well as processes shown in FIG. 1 (in particular, e.g., FIG. 8B and/or FIG. 8C) as Step B) is shortened to 1 to 7 days and the rapid second expansion (including processes referred to herein as Rapid Expansion Protocol (REP) as well as processes shown in FIG. 1 (in particular, e.g., FIG. 8B and/or FIG. 8C) as Step D) is shortened to 1 to 9 days, as discussed in detail below as well as in the examples and figures. In some embodiments, the priming first expansion (including processes referred herein as the pre-Rapid Expansion (Pre-REP), as well as processes shown in FIG. 1 (in particular, e.g., FIG. 1B and/or FIG. 8C) as Step B) is 1 to 7 days and the rapid second expansion (including processes referred to herein as Rapid Expansion Protocol (REP) as well as processes shown in FIG. 1 (in particular, e.g., FIG. 8B and/or FIG. 8C) as Step D) is 1 to 10 days, as discussed in detail below as well as in the examples and figures. In some embodiments, the priming first expansion (for example, an expansion described as Step B in FIG. 1 (in particular, e.g., FIG. 8B and/or FIG. 8C)) is shortened to 8 days and the rapid second expansion (for example, an expansion as described in Step D in FIG. 1 (in particular, e.g., FIG. 8B and/or FIG. 8C)) is 7 to 9 days. In some embodiments, the priming first expansion (for example, an expansion described as Step B in FIG. 1 (in particular, e.g., FIG. 8B and/or FIG. 8C)) is 8 days and the rapid second expansion (for example, an expansion as described in Step D in FIG. 1 (in particular, e.g., FIG. 8B and/or FIG. 8C)) is 8 to 9 days. In some embodiments, the priming first expansion (for example, an expansion described as Step B in FIG. 1 (in particular, e.g., FIG. 8B and/or FIG. 8C)) is shortened to 7 days and the rapid second expansion (for example, an expansion as described in Step D in FIG. 1 (in particular, e.g., FIG. 8B and/or FIG. 8C)) is 7 to 8 days. In some embodiments, the priming first expansion (for example, an expansion described as Step B in FIG. 8 (in particular, e.g., FIG. 8B and/or FIG. 8C)) is shortened to 8 days and the rapid second expansion (for example, an expansion as described in Step D in FIG. 8 (in particular, e.g., FIG. 8B and/or FIG. 8C)) is 8 days. In some embodiments, the priming first expansion (for example, an expansion described as Step B in FIG. 8 (in particular, e.g., FIG. 8B and/or FIG. 8C)) is 8 days and the rapid second expansion (for example, an expansion as described in Step D in FIG. 8 (in particular, e.g., FIG. 8B and/or FIG. 8C)) is 9 days. In some embodiments, the priming first expansion (for example, an expansion described as Step B in FIG. 8 (in particular, e.g., FIG. 8B and/or FIG. 8C)) is 8 days and the rapid second expansion (for example, an expansion as described in Step D in FIG. 8 (in particular, e.g., FIG. 8B and/or FIG. 8C)) is 10 days. In some embodiments, the priming first expansion (for example, an expansion described as Step B in FIG. 8 (in particular, e.g., FIG. 8B and/or FIG. 8C)) is 7 days and the rapid second expansion (for example, an expansion as described in Step D in FIG. 8 (in particular, e.g., FIG. 8B and/or FIG. 8C)) is 7 to 10 days. In some embodiments, the priming first expansion (for example, an expansion described as Step B in FIG. 8 (in particular, e.g., FIG. 8B and/or FIG. 8C)) is 7 days and the rapid second expansion (for example, an expansion as described in Step D in FIG. 8 (in particular, e.g., FIG. 8B and/or FIG. 8C)) is 8 to 10 days. In some embodiments, the priming first expansion (for example, an expansion described as Step B in FIG. 8 (in particular, e.g., FIG. 8B and/or FIG. 8C)) is 7 days and the rapid second expansion (for example, an expansion as described in Step D in FIG. 8 (in particular, e.g., FIG. 8B and/or FIG. 8C)) is 9 to 10 days. In some embodiments, the priming first expansion (for example, an expansion described as Step B in FIG. 8 (in particular, e.g., FIG. 8B and/or FIG. 8C)) is shortened to 7 days and the rapid second expansion (for example, an expansion as described in Step D in FIG. 8 (in particular, e.g., FIG. 8B and/or FIG. 8C)) is 7 to 9 days. In some embodiments, the combination of the priming first expansion and rapid second expansion (for example, expansions described as Step B and Step D in FIG. 1 (in particular, e.g., FIG. 1B and/or FIG. 8C)) is 14-16 days, as discussed in detail below and in the examples and figures. Particularly, it is considered that certain embodiments of the present invention comprise a priming first expansion step in which TILs are activated by exposure to an anti-CD3 antibody, e.g., OKT-3 in the presence of IL-2 or exposure to an antigen in the presence of at least IL-2 and an anti-CD3 antibody e.g. OKT-3. In certain embodiments, the TILs which are activated in the priming first expansion step as described above are a first population of TILs i.e., which are a primary cell population.

The “Step” Designations A, B, C, etc., below are in reference to the non-limiting example in FIG. 8 (in particular, e.g., FIG. 8B and/or FIG. 8C) and in reference to certain non-limiting embodiments described herein. The ordering of the Steps below and in FIG. 8 (in particular, e.g., FIG. 8B and/or FIG. 8C) is exemplary and any combination or order of steps, as well as additional steps, repetition of steps, and/or omission of steps is contemplated by the present application and the methods disclosed herein.

### A. STEP A: Obtain Patient Tumor Sample

In general, TILs are initially obtained from a patient tumor sample (“primary TILs”) or from circulating lymphocytes, such as peripheral blood lymphocytes, including peripheral blood lymphocytes having TIL-like characteristics, and are then expanded into a larger population for further manipulation as described herein, optionally cryopreserved, and optionally evaluated for phenotype and metabolic parameters as an indication of TIL health.

A patient tumor sample may be obtained using methods known in the art, generally via surgical resection, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma). In some embodiments, the cancer is selected from cervical cancer, head and neck cancer (including, for example, head and neck squamous cell carcinoma (HNSCC)), glioblastoma (GBM), gastrointestinal cancer, ovarian cancer, sarcoma, pancreatic cancer, bladder cancer, breast cancer, triple negative breast cancer, and non-small cell lung carcinoma. In some embodiments, useful TILs are obtained from malignant melanoma tumors, as these have been reported to have particularly high levels of TILs.

Once obtained, the tumor sample is generally fragmented using sharp dissection into small pieces of between 1 to about 8 mm³, with from about 2-3 mm³ being particularly useful. The TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

As indicated above, in some embodiments, the TILs are derived from solid tumors. In some embodiments, the solid tumors are not fragmented. In some embodiments, the solid tumors are not fragmented and are subjected to enzymatic digestion as whole tumors. In some embodiments, the tumors are digested in in an enzyme mixture comprising collagenase, DNase, and hyaluronidase. In some embodiments, the tumors are digested in in an enzyme mixture comprising collagenase, DNase, and hyaluronidase for 1-2 hours. In some embodiments, the tumors are digested in in an enzyme mixture comprising collagenase, DNase, and hyaluronidase for 1-2 hours at 37° C., 5% CO₂. In some embodiments, the tumors are digested in in an enzyme mixture comprising collagenase, DNase, and hyaluronidase for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture.

In some embodiments, the tumor is reconstituted with the lyophilized enzymes in a sterile buffer. In some embodiments, the buffer is sterile HBSS.

In some embodiments, the enzyme mixture comprises collagenase. In some embodiments, the collagenase is collagenase IV. In some embodiments, the working stock for the collagenase is a 100 mg/mL 10× working stock.

In some embodiments, the enzyme mixture comprises DNAse. In some embodiments, the working stock for the DNAse is a 10,000 IU/mL 10× working stock.

In some embodiments, the enzyme mixture comprises hyaluronidase. In some embodiments, the working stock for the hyaluronidase is a 10-mg/mL 10× working stock.

In some embodiments, the enzyme mixture comprises 10 mg/mL collagenase, 1000 IU/mL DNAse, and 1 mg/mL hyaluronidase.

In some embodiments, the enzyme mixture comprises 10 mg/mL collagenase, 500 IU/mL DNAse, and 1 mg/mL hyaluronidase.

In general, the cell suspension obtained from the tumor is called a “primary cell population” or a “freshly obtained” or a “freshly isolated” cell population. In certain embodiments, the freshly obtained cell population of TILs is exposed to a cell culture medium comprising antigen presenting cells, IL-12 and OKT-3.

In some embodiments, fragmentation includes physical fragmentation, including, for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from patients. In an embodiment, TILs can be initially cultured from enzymatic tumor digests and tumor fragments obtained from patients.

In some embodiments, where the tumor is a solid tumor, the tumor undergoes physical fragmentation after the tumor sample is obtained in, for example, Step A (as provided in FIG. 8 (in particular, e.g., FIG. 8B and/or FIG. 8C)). In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the step of fragmentation is an in vitro or ex-vivo process. In some embodiments, the tumor is fragmented and 10, 20, 30, 40 or more fragments or pieces are placed in each container for the priming first expansion. In some embodiments, the tumor is fragmented and 30 or 40 fragments or pieces are placed in each container for the priming first expansion. In some embodiments, the tumor is fragmented and 40 fragments or pieces are placed in each container for the priming first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumor fragments are 1-4 mm×1-4 mm×1-4 mm. In some embodiments, the tumor fragments are 1 mm×1 mm×1 mm. In some embodiments, the tumor fragments are 2 mm×2 mm×2 mm. In some embodiments, the tumor fragments are 3 mm×3 mm×3 mm. In some embodiments, the tumor fragments are 4 mm×4 mm×4 mm.

In some embodiments, the tumors are fragmented in order to minimize the amount of hemorrhagic, necrotic, and/or fatty tissues on each piece. In some embodiments, the tumors are fragmented in order to minimize the amount of hemorrhagic tissue on each piece. In some embodiments, the tumors are fragmented in order to minimize the amount of necrotic tissue on each piece. In some embodiments, the tumors are fragmented in order to minimize the amount of fatty tissue on each piece. In certain embodiments, the step of fragmentation of the tumor is an in vitro or ex-vivo method.

In some embodiments, the tumor fragmentation is performed in order to maintain the tumor internal structure. In some embodiments, the tumor fragmentation is performed without preforming a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, the cell suspension prior to the priming first expansion step is called a “primary cell population” or a “freshly obtained” or “freshly isolated” cell population.

In some embodiments, cells can be optionally frozen after sample isolation (e.g., after obtaining the tumor sample and/or after obtaining the cell suspension from the tumor sample) and stored frozen prior to entry into the expansion described in Step B, which is described in further detail below, as well as exemplified in FIG. 8 (in particular, e.g., FIG. 8B).

### 1. Core/Small Biopsy Derived TILs

In some embodiments, TILs are initially obtained from a patient tumor sample (“primary TILs”) obtained by a core biopsy or similar procedure and then expanded into a larger population for further manipulation as described herein, optionally cryopreserved, and optionally evaluated for phenotype and metabolic parameters.

In some embodiments, a patient tumor sample may be obtained using methods known in the art, generally via small biopsy, core biopsy, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. In some embodiments, the sample can be from multiple small tumor samples or biopsies. In some embodiments, the sample can comprise multiple tumor samples from a single tumor from the same patient. In some embodiments, the sample can comprise multiple tumor samples from one, two, three, or four tumors from the same patient. In some embodiments, the sample can comprise multiple tumor samples from multiple tumors from the same patient. The solid tumor may of lung and/or non-small cell lung carcinoma (NSCLC).

In general, the cell suspension obtained from the tumor core or fragment is called a “primary cell population” or a “freshly obtained” or a “freshly isolated” cell population. In certain embodiments, the freshly obtained cell population of TILs is exposed to a cell culture medium comprising antigen presenting cells, IL-2 and OKT-3.

In some embodiments, if the tumor is metastatic and the primary lesion has been efficiently treated/removed in the past, removal of one of the metastatic lesions may be needed. In some embodiments, the least invasive approach is to remove a skin lesion, or a lymph node on the neck or axillary area when available. In some embodiments, a skin lesion is removed or small biopsy thereof is removed. In some embodiments, a lymph node or small biopsy thereof is removed. In some embodiments, a lung or liver metastatic lesion, or an intra-abdominal or thoracic lymph node or small biopsy can thereof can be employed.

In some embodiments, the tumor is a melanoma. In some embodiments, the small biopsy for a melanoma comprises a mole or portion thereof.

In some embodiments, the small biopsy is a punch biopsy. In some embodiments, the punch biopsy is obtained with a circular blade pressed into the skin. In some embodiments, the punch biopsy is obtained with a circular blade pressed into the skin, around a suspicious mole. In some embodiments, the punch biopsy is obtained with a circular blade pressed into the skin, and a round piece of skin is removed. In some embodiments, the small biopsy is a punch biopsy and round portion of the tumor is removed.

In some embodiments, the small biopsy is an excisional biopsy. In some embodiments, the small biopsy is an excisional biopsy and the entire mole or growth is removed. In some embodiments, the small biopsy is an excisional biopsy and the entire mole or growth is removed along with a small border of normal-appearing skin.

In some embodiments, the small biopsy is an incisional biopsy. In some embodiments, the small biopsy is an incisional biopsy and only the most irregular part of a mole or growth is taken. In some embodiments, the small biopsy is an incisional biopsy and the incisional biopsy is used when other techniques can't be completed, such as if a suspicious mole is very large.

In some embodiments, the small biopsy is a lung biopsy. In some embodiments, the small biopsy is obtained by bronchoscopy. Generally, bronchoscopy, the patient is put under anesthesia, and a small tool goes through the nose or mouth, down the throat, and into the bronchial passages, where small tools are used to remove some tissue. In some embodiments, where the tumor or growth cannot be reached via bronchoscopy, a transthoracic needle biopsy can be employed. Generally, for a transthoracic needle biopsy, the patient is also under anesthesia and a needle is inserted through the skin directly into the suspicious spot to remove a small sample of tissue. In some embodiments, a transthoracic needle biopsy may require interventional radiology (for example, the use of x-rays or CT scan to guide the needle). In some embodiments, the small biopsy is obtained by needle biopsy. In some embodiments, the small biopsy is obtained endoscopic ultrasound (for example, an endoscope with a light and is placed through the mouth into the esophagus). In some embodiments, the small biopsy is obtained surgically.

In some embodiments, the small biopsy is a head and neck biopsy. In some embodiments, the small biopsy is an incisional biopsy. In some embodiments, the small biopsy is an incisional biopsy, wherein a small piece of tissue is cut from an abnormal-looking area. In some embodiments, if the abnormal region is easily accessed, the sample may be taken without hospitalization. In some embodiments, if the tumor is deeper inside the mouth or throat, the biopsy may need to be done in an operating room, with general anesthesia. In some embodiments, the small biopsy is an excisional biopsy. In some embodiments, the small biopsy is an excisional biopsy, wherein the whole area is removed. In some embodiments, the small biopsy is a fine needle aspiration (FNA). In some embodiments, the small biopsy is a fine needle aspiration (FNA), wherein a very thin needle attached to a syringe is used to extract (aspirate) cells from a tumor or lump. In some embodiments, the small biopsy is a punch biopsy. In some embodiments, the small biopsy is a punch biopsy, wherein punch forceps are used to remove a piece of the suspicious area.

In some embodiments, the small biopsy is a cervical biopsy. In some embodiments, the small biopsy is obtained via colposcopy. Generally, colposcopy methods employ the use of a lighted magnifying instrument attached to magnifying binoculars (a colposcope) which is then used to biopsy a small section of the surface of the cervix. In some embodiments, the small biopsy is a conization/cone biopsy. In some embodiments, the small biopsy is a conization/cone biopsy, wherein an outpatient surgery may be needed to remove a larger piece of tissue from the cervix. In some embodiments, the cone biopsy, in addition to helping to confirm a diagnosis, a cone biopsy can serve as an initial treatment.

The term “solid tumor” refers to an abnormal mass of tissue that usually does not contain cysts or liquid areas. Solid tumors may be benign or malignant. The term “solid tumor cancer refers to malignant, neoplastic, or cancerous solid tumors. Solid tumor cancers include cancers of the lung. In some embodiments, the cancer is non-small cell lung carcinoma (NSCLC). The tissue structure of solid tumors includes interdependent tissue compartments including the parenchyma (cancer cells) and the supporting stromal cells in which the cancer cells are dispersed and which may provide a supporting microenvironment.

In some embodiments, the sample from the tumor is obtained as a fine needle aspirate (FNA), a core biopsy, a small biopsy (including, for example, a punch biopsy). In some embodiments, sample is placed first into a G-Rex 10. In some embodiments, sample is placed first into a G-Rex 10 when there are 1 or 2 core biopsy and/or small biopsy samples. In some embodiments, sample is placed first into a G-Rex 100 when there are 3, 4, 5, 6, 8, 9, or 10 or more core biopsy and/or small biopsy samples. In some embodiments, sample is placed first into a G-Rex 500 when there are 3, 4, 5, 6, 8, 9, or 10 or more core biopsy and/or small biopsy samples.

The FNA can be obtained from a lung tumor, including, for example, an NSCLC. In some embodiments, the FNA is obtained from a lung tumor, such as a lung tumor from a patient with non-small cell lung cancer (NSCLC). In some cases, the patient with NSCLC has previously undergone a surgical treatment.

TILs described herein can be obtained from an FNA sample. In some cases, the FNA sample is obtained or isolated from the patient using a fine gauge needle ranging from an 18 gauge needle to a 25 gauge needle. The fine gauge needle can be 18 gauge, 19 gauge, 20 gauge, 21 gauge, 22 gauge, 23 gauge, 24 gauge, or 25 gauge. In some embodiments, the FNA sample from the patient can contain at least 400,000 TILs, e.g., 400,000 TILs, 450,000 TILs, 500,000 TILs, 550,000 TILs, 600,000 TILs, 650,000 TILs, 700,000 TILs, 750,000 TILs, 800,000 TILs, 850,000 TILs, 900,000 TILs, 950,000 TILs, or more.

In some cases, the TILs described herein are obtained from a core biopsy sample. In some cases, the core biopsy sample is obtained or isolated from the patient using a surgical or medical needle ranging from an 11 gauge needle to a 16 gauge needle. The needle can be 11 gauge, 12 gauge, 13 gauge, 14 gauge, 15 gauge, or 16 gauge. In some embodiments, the core biopsy sample from the patient can contain at least 400,000 TILs, e.g., 400,000 TILs, 450,000 TILs, 500,000 TILs, 550,000 TILs, 600,000 TILs, 650,000 TILs, 700,000 TILs, 750,000 TILs, 800,000 TILs, 850,000 TILs, 900,000 TILs, 950,000 TILs, or more.

In general, the harvested cell suspension is called a “primary cell population” or a “freshly harvested” cell population.

In some embodiments, the TILs are not obtained from tumor digests. In some embodiments, the solid tumor cores are not fragmented.

In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, obtaining the first population of TILs comprises a multilesional sampling method.

Tumor dissociating enzyme mixtures can include one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof.

In some embodiments, the dissociating enzymes are reconstituted from lyophilized enzymes. In some embodiments, lyophilized enzymes are reconstituted in an amount of sterile buffer such as Hank's balance salt solution (HBSS).

In some instances, collagenase (such as animal free-type 1 collagenase) is reconstituted in 10 mL of sterile HBSS or another buffer. The lyophilized stock enzyme may be at a concentration of 2892 PZ U/vial. In some embodiments, collagenase is reconstituted in 5 mL to 15 mL buffer. In some embodiment, after reconstitution the collagenase stock ranges from about 100 PZ U/mL-about 400 PZ U/mL, e.g., about 100 PZ U/mL-about 400 PZ U/mL, about 100 PZ U/mL-about 350 PZ U/mL, about 100 PZ U/mL-about 300 PZ U/mL, about 150 PZ U/mL-about 400 PZ U/mL, about 100 PZ U/mL, about 150 PZ U/mL, about 200 PZ U/mL, about 210 PZ U/mL, about 220 PZ U/mL, about 230 PZ U/mL, about 240 PZ U/mL, about 250 PZ U/mL, about 260 PZ U/mL, about 270 PZ U/mL, about 280 PZ U/mL, about 289.2 PZ U/mL, about 300 PZ U/mL, about 350 PZ U/mL, or about 400 PZ U/mL.

In some embodiments neutral protease is reconstituted in 1 mL of sterile HBSS or another buffer. The lyophilized stock enzyme may be at a concentration of 175 DMC U/vial. In some embodiments, after reconstitution the neutral protease stock ranges from about 100 DMC/mL-about 400 DMC/mL, e.g., about 100 DMC/mL-about 400 DMC/mL, about 100 DMC/mL-about 350 DMC/mL, about 100 DMC/mL-about 300 DMC/mL, about 150 DMC/mL-about 400 DMC/mL, about 100 DMC/mL, about 110 DMC/mL, about 120 DMC/mL, about 130 DMC/mL, about 140 DMC/mL, about 150 DMC/mL, about 160 DMC/mL, about 170 DMC/mL, about 175 DMC/mL, about 180 DMC/mL, about 190 DMC/mL, about 200 DMC/mL, about 250 DMC/mL, about 300 DMC/mL, about 350 DMC/mL, or about 400 DMC/mL.

In some embodiments, DNAse I is reconstituted in 1 mL of sterile HBSS or another buffer. The lyophilized stock enzyme was at a concentration of 4 KU/vial. In some embodiments, after reconstitution the DNase I stock ranges from about 1 KU/mL to 10 KU/mL, e.g., about 1 KU/mL, about 2 KU/mL, about 3 KU/mL, about 4 KU/mL, about 5 KU/mL, about 6 KU/mL, about 7 KU/mL, about 8 KU/mL, about 9 KU/mL, or about 10 KU/mL.

In some embodiments, the stock of enzymes could change so verify the concentration of the lyophilized stock and amend the final amount of enzyme added to the digest cocktail accordingly

In some embodiments, the enzyme mixture includes about 10.2-ul of neutral protease (0.36 DMC U/mL), 21.3-ul of collagenase (1.2 PZ/mL) and 250-ul of DNAse 1 (200 U/mL) in about 4.7 mL of sterile HBSS.

### 2. Pleural Effusion T-Cells and TILs

In some embodiments, the sample is a pleural fluid sample. In some embodiments, the source of the T-cells or TILs for expansion according to the processes described herein is a pleural fluid sample. In some embodiments, the sample is a pleural effusion derived sample. In some embodiments, the source of the T-cells or TILs for expansion according to the processes described herein is a pleural effusion derived sample. See, for example, methods described in U.S. Patent Publication US 2014/0295426, incorporated herein by reference in its entirety for all purposes.

In some embodiments, any pleural fluid or pleural effusion suspected of and/or containing TILs can be employed. Such a sample may be derived from a primary or metastatic lung cancer, such as NSCLC or SCLC. In some embodiments, the sample may be secondary metastatic cancer cells which originated from another organ, e.g., breast, ovary, colon or prostate. In some embodiments, the sample for use in the expansion methods described herein is a pleural exudate. In some embodiments, the sample for use in the expansion methods described herein is a pleural transudate. Other biological samples may include other serous fluids containing TILs, including, e.g., ascites fluid from the abdomen or pancreatic cyst fluid. Ascites fluid and pleural fluids involve very similar chemical systems; both the abdomen and lung have mesothelial lines and fluid forms in the pleural space and abdominal spaces in the same matter in malignancies and such fluids in some embodiments contain TILs. In some embodiments, wherein the disclosure exemplifies pleural fluid, the same methods may be performed with similar results using ascites or other cyst fluids containing TILs.

In some embodiments, the pleural fluid is in unprocessed form, directly as removed from the patient. In some embodiments, the unprocessed pleural fluid is placed in a standard blood collection tube, such as an EDTA or Heparin tube, prior to the contacting step. In some embodiments, the unprocessed pleural fluid is placed in a standard CellSave® tube (Veridex) prior to the contacting step. In some embodiments, the sample is placed in the CellSave tube immediately after collection from the patient to avoid a decrease in the number of viable TILs. The number of viable TILs can decrease to a significant extent within 24 hours, if left in the untreated pleural fluid, even at 4° C. In some embodiments, the sample is placed in the appropriate collection tube within 1 hour, 5 hours, 10 hours, 15 hours, or up to 24 hours after removal from the patient. In some embodiments, the sample is placed in the appropriate collection tube within 1 hour, 5 hours, 10 hours, 15 hours, or up to 24 hours after removal from the patient at 4° C.

In some embodiments, the pleural fluid sample from the chosen subject may be diluted. In one embodiment, the dilution is 1:10 pleural fluid to diluent. In another embodiment, the dilution is 1:9 pleural fluid to diluent. In another embodiment, the dilution is 1:8 pleural fluid to diluent. In another embodiment, the dilution is 1:5 pleural fluid to diluent. In another embodiment, the dilution is 1:2 pleural fluid to diluent. In another embodiment, the dilution is 1:1 pleural fluid to diluent. In some embodiments, diluents include saline, phosphate buffered saline, another buffer or a physiologically acceptable diluent. In some embodiments, the sample is placed in the CellSave tube immediately after collection from the patient and dilution to avoid a decrease in the viable TILs, which may occur to a significant extent within 24-48 hours, if left in the untreated pleural fluid, even at 4° C. In some embodiments, the pleural fluid sample is placed in the appropriate collection tube within 1 hour, 5 hours, 10 hours, 15 hours, 24 hours, 36 hours, up to 48 hours after removal from the patient, and dilution. In some embodiments, the pleural fluid sample is placed in the appropriate collection tube within 1 hour, 5 hours, 10 hours, 15 hours, 24 hours, 36 hours, up to 48 hours after removal from the patient, and dilution at 4° C.

In still another embodiment, pleural fluid samples are concentrated by conventional means prior further processing steps. In some embodiments, this pre-treatment of the pleural fluid is preferable in circumstances in which the pleural fluid must be cryopreserved for shipment to a laboratory performing the method or for later analysis (e.g., later than 24-48 hours post-collection). In some embodiments, the pleural fluid sample is prepared by centrifuging the pleural fluid sample after its withdrawal from the subject and resuspending the centrifugate or pellet in buffer. In some embodiments, the pleural fluid sample is subjected to multiple centrifugations and resuspensions, before it is cryopreserved for transport or later analysis and/or processing.

In some embodiments, pleural fluid samples are concentrated prior to further processing steps by using a filtration method. In some embodiments, the pleural fluid sample used in the contacting step is prepared by filtering the fluid through a filter containing a known and essentially uniform pore size that allows for passage of the pleural fluid through the membrane but retains the tumor cells. In some embodiments, the diameter of the pores in the membrane may be at least 4 μM. In another embodiment the pore diameter may be 5 μM or more, and in other embodiment, any of 6, 7, 8, 9, or 10 μM. After filtration, the cells, including TILs, retained by the membrane may be rinsed off the membrane into a suitable physiologically acceptable buffer. Cells, including TILs, concentrated in this way may then be used in the contacting step of the method.

In some embodiments, pleural fluid sample (including, for example, the untreated pleural fluid), diluted pleural fluid, or the resuspended cell pellet, is contacted with a lytic reagent that differentially lyses non-nucleated red blood cells present in the sample. In some embodiments, this step is performed prior to further processing steps in circumstances in which the pleural fluid contains substantial numbers of RBCs. Suitable lysing reagents include a single lytic reagent or a lytic reagent and a quench reagent, or a lytic agent, a quench reagent and a fixation reagent. Suitable lytic systems are marketed commercially and include the BD Pharm Lyse™ system (Becton Dickenson). Other lytic systems include the Versalyse™ system, the FACSlyse™ system (Becton Dickenson), the Immunoprep™ system or Erythrolyse II system (Beckman Coulter, Inc.), or an ammonium chloride system. In some embodiments, the lytic reagent can vary with the primary requirements being efficient lysis of the red blood cells, and the conservation of the TILs and phenotypic properties of the TILs in the pleural fluid. In addition to employing a single reagent for lysis, the lytic systems useful in methods described herein can include a second reagent, e.g., one that quenches or retards the effect of the lytic reagent during the remaining steps of the method, e.g., Stabilyse™ reagent (Beckman Coulter, Inc.). A conventional fixation reagent may also be employed depending upon the choice of lytic reagents or the preferred implementation of the method.

In some embodiments, the pleural fluid sample, unprocessed, diluted or multiply centrifuged or processed as described herein above is cryopreserved at a temperature of about −140° C. prior to being further processed and/or expanded as provided herein.

3. Methods of Expanding Peripheral Blood Lymphocytes (PBLs) from Peripheral Blood

PBL Method 1. In an embodiment of the invention, PBLs are expanded using the processes described herein. In an embodiment of the invention, the method comprises obtaining a PBMC sample from whole blood. In an embodiment, the method comprises enriching T-cells by isolating pure T-cells from PBMCs using negative selection of a non-CD19+ fraction. In an embodiment, the method comprises enriching T-cells by isolating pure T-cells from PBMCs using magnetic bead-based negative selection of a non-CD19+ fraction.

In an embodiment of the invention, PBL Method 1 is performed as follows: On Day 0, a cryopreserved PBMC sample is thawed and PBMCs are counted. T-cells are isolated using a Human Pan T-Cell Isolation Kit and LS columns (Miltenyi Biotec).

PBL Method 2. In an embodiment of the invention, PBLs are expanded using PBL Method 2, which comprises obtaining a PBMC sample from whole blood. The T-cells from the PBMCs are enriched by incubating the PBMCs for at least three hours at 37° C. and then isolating the non-adherent cells.

In an embodiment of the invention, PBL Method 2 is performed as follows: On Day 0, the cryopreserved PMBC sample is thawed and the PBMC cells are seeded at 6 million cells per well in a 6 well plate in CM-2 media and incubated for 3 hours at 37 degrees Celsius. After 3 hours, the non-adherent cells, which are the PBLs, are removed and counted.

PBL Method 3. In an embodiment of the invention, PBLs are expanded using PBL Method 3, which comprises obtaining a PBMC sample from peripheral blood. B-cells are isolated using a CD19+ selection and T-cells are selected using negative selection of the non-CD19+ fraction of the PBMC sample.

In an embodiment of the invention, PBL Method 3 is performed as follows: On Day 0, cryopreserved PBMCs derived from peripheral blood are thawed and counted. CD19+ B-cells are sorted using a CD19 Multisort Kit, Human (Miltenyi Biotec). Of the non-CD19+ cell fraction, T-cells are purified using the Human Pan T-cell Isolation Kit and LS Columns (Miltenyi Biotec).

In an embodiment, PBMCs are isolated from a whole blood sample. In an embodiment, the PBMC sample is used as the starting material to expand the PBLs. In an embodiment, the sample is cryopreserved prior to the expansion process. In another embodiment, a fresh sample is used as the starting material to expand the PBLs. In an embodiment of the invention, T-cells are isolated from PBMCs using methods known in the art. In an embodiment, the T-cells are isolated using a Human Pan T-cell isolation kit and LS columns. In an embodiment of the invention, T-cells are isolated from PBMCs using antibody selection methods known in the art, for example, CD19 negative selection.

In an embodiment of the invention, the PBMC sample is incubated for a period of time at a desired temperature effective to identify the non-adherent cells. In an embodiment of the invention, the incubation time is about 3 hours. In an embodiment of the invention, the temperature is about 37° Celsius. The non-adherent cells are then expanded using the process described above.

In some embodiments, the PBMC sample is from a subject or patient who has been optionally pre-treated with a regimen comprising a kinase inhibitor or an ITK inhibitor. In some embodiments, the tumor sample is from a subject or patient who has been pre-treated with a regimen comprising a kinase inhibitor or an ITK inhibitor. In some embodiments, the PBMC sample is from a subject or patient who has been pre-treated with a regimen comprising a kinase inhibitor or an ITK inhibitor, has undergone treatment for at least 1 month, at least 2 months, at least 3 months, at least 4 months, at least 5 months, at least 6 months, or 1 year or more. In another embodiment, the PBMCs are derived from a patient who is currently on an ITK inhibitor regimen, such as ibrutinib.

In some embodiments, the PBMC sample is from a subject or patient who has been pre-treated with a regimen comprising a kinase inhibitor or an ITK inhibitor and is refractory to treatment with a kinase inhibitor or an ITK inhibitor, such as ibrutinib.

In some embodiments, the PBMC sample is from a subject or patient who has been pre-treated with a regimen comprising a kinase inhibitor or an ITK inhibitor but is no longer undergoing treatment with a kinase inhibitor or an ITK inhibitor. In some embodiments, the PBMC sample is from a subject or patient who has been pre-treated with a regimen comprising a kinase inhibitor or an ITK inhibitor but is no longer undergoing treatment with a kinase inhibitor or an ITK inhibitor and has not undergone treatment for at least 1 month, at least 2 months, at least 3 months, at least 4 months, at least 5 months, at least 6 months, or at least 1 year or more. In another embodiment, the PBMCs are derived from a patient who has prior exposure to an ITK inhibitor, but has not been treated in at least 3 months, at least 6 months, at least 9 months, or at least 1 year.

In an embodiment of the invention, at Day 0, cells are selected for CD19+ and sorted accordingly. In an embodiment of the invention, the selection is made using antibody binding beads. In an embodiment of the invention, pure T-cells are isolated on Day 0 from the PBMCs.

In an embodiment of the invention, for patients that are not pre-treated with ibrutinib or other ITK inhibitor, 10-15 mL of Buffy Coat will yield about 5×10⁹ PBMC, which, in turn, will yield about 5.5×10⁷ PBLs.

In an embodiment of the invention, for patients that are pre-treated with ibrutinib or other ITK inhibitor, the expansion process will yield about 20×10⁹ PBLs. In an embodiment of the invention, 40.3×10⁶ PBMCs will yield about 4.7×10⁵ PBLs.

In any of the foregoing embodiments, PBMCs may be derived from a whole blood sample, by apheresis, from the buffy coat, or from any other method known in the art for obtaining PBMCs.

In any of the foregoing embodiments, the PBLs may be genetically modified to express the CCRs described herein. In some embodiments, PBLs are prepared using the methods described in U.S. Patent Application Publication No. US 2020/0347350 A1, the disclosures of which are incorporated by reference herein.

4. Methods of Expanding Marrow Infiltrating Lymphocytes (MILs) from PBMCs Derived from Bone Marrow

MIL Method 3. In an embodiment of the invention, the method comprises obtaining PBMCs from the bone marrow. On Day 0, the PBMCs are selected for CD3+/CD33+/CD20+/CD14+ and sorted, and the non-CD3+/CD33+/CD20+/CD14+ cell fraction is sonicated and a portion of the sonicated cell fraction is added back to the selected cell fraction.

In an embodiment of the invention, MIL Method 3 is performed as follows: On Day 0, a cryopreserved sample of PBMCs is thawed and PBMCs are counted. The cells are stained with CD3, CD33, CD20, and CD14 antibodies and sorted using a S3e cell sorted (Bio-Rad). The cells are sorted into two fractions—an immune cell fraction (or the MIL fraction) (CD3+ CD33+ CD20+ CD14+) and an AML blast cell fraction (non-CD3+ CD33+ CD20+ CD14+).

In an embodiment of the invention, PBMCs are obtained from bone marrow. In an embodiment, the PBMCs are obtained from the bone marrow through apheresis, aspiration, needle biopsy, or other similar means known in the art. In an embodiment, the PBMCs are fresh. In another embodiment, the PBMCs are cryopreserved.

In an embodiment of the invention, MILs are expanded from 10-50 mL of bone marrow aspirate. In an embodiment of the invention, 10 mL of bone marrow aspirate is obtained from the patient. In another embodiment, 20 mL of bone marrow aspirate is obtained from the patient. In another embodiment, 30 mL of bone marrow aspirate is obtained from the patient. In another embodiment, 40 mL of bone marrow aspirate is obtained from the patient. In another embodiment, 50 mL of bone marrow aspirate is obtained from the patient.

In an embodiment of the invention, the number of PBMCs yielded from about 10-50 mL of bone marrow aspirate is about 5×10⁷ to about 10×10⁷ PBMCs. In another embodiment, the number of PMBCs yielded is about 7×10⁷ PBMCs.

In an embodiment of the invention, about 5×10⁷ to about 10×10⁷ PBMCs, yields about 0.5×10⁶ to about 1.5×10⁶ MILs. In an embodiment of the invention, about 1×10⁶ MILs is yielded.

In an embodiment of the invention, 12×10⁶ PBMC derived from bone marrow aspirate yields approximately 1.4×10⁵ MILs.

In any of the foregoing embodiments, PBMCs may be derived from a whole blood sample, from bone marrow, by apheresis, from the buffy coat, or from any other method known in the art for obtaining PBMCs.

In any of the foregoing embodiments, the MILs may be genetically modified to express the CCRs described herein. In some embodiments, MILs are prepared using the methods described in U.S. Patent Application Publication No. US 2020/0347350 A1, the disclosures of which are incorporated by reference herein.

### B. STEP B: Priming First Expansion

In some embodiments, the present methods provide for younger TILs, which may provide additional therapeutic benefits over older TILs (i.e., TILs which have further undergone more rounds of replication prior to administration to a subject/patient). Features of young TILs have been described in the literature, for example in Donia, et al., Scand. J Immunol. 2012, 75, 157-167; Dudley, et al., Clin. Cancer Res. 2010, 16, 6122-6131; Huang, et al., J. Immunother. 2005, 28, 258-267; Besser, et al., Clin. Cancer Res. 2013, 19, OF1-OF9; Besser, et al., J. Immunother. 2009, 32, 415-423; Robbins, et al., J. Immunol. 2004, 173, 7125-7130; Shen, et al., J. Immunother., 2007, 30, 123-129; Zhou, et al., J. Immunother. 2005, 28, 53-62; and Tran, et al., J. Immunother., 2008, 31, 742-751, each of which is incorporated herein by reference.

After dissection or digestion of tumor fragments and/or tumor fragments, for example such as described in Step A of FIG. 1 (in particular, e.g., FIG. 1B and/or FIG. 8C), the resulting cells are cultured in serum containing IL-2, OKT-3, and feeder cells (e.g., antigen-presenting feeder cells), under conditions that favor the growth of TILs over tumor and other cells. In some embodiments, the IL-2, OKT-3, and feeder cells are added at culture initiation along with the tumor digest and/or tumor fragments (e.g., at Day 0). In some embodiments, the tumor digests and/or tumor fragments are incubated in a container with up to 60 fragments per container and with 6000 IU/mL of IL-2. In some embodiments, this primary cell population is cultured for a period of days, generally from 1 to 8 days, resulting in a bulk TIL population, generally about 1×10⁸ bulk TIL cells. In some embodiments, this primary cell population is cultured for a period of days, generally from 1 to 7 days, resulting in a bulk TIL population, generally about 1×10⁸ bulk TIL cells. In some embodiments, priming first expansion occurs for a period of 1 to 8 days, resulting in a bulk TIL population, generally about 1×10⁸ bulk TIL cells. In some embodiments, priming first expansion occurs for a period of 1 to 7 days, resulting in a bulk TIL population, generally about 1×10⁸ bulk TIL cells. In some embodiments, this priming first expansion occurs for a period of 5 to 8 days, resulting in a bulk TIL population, generally about 1×10⁸ bulk TIL cells. In some embodiments, this priming first expansion occurs for a period of 5 to 7 days, resulting in a bulk TIL population, generally about 1×10⁸ bulk TIL cells. In some embodiments, this priming first expansion occurs for a period of about 6 to 8 days, resulting in a bulk TIL population, generally about 1×10⁸ bulk TIL cells. In some embodiments, this priming first expansion occurs for a period of about 6 to 7 days, resulting in a bulk TIL population, generally about 1×10⁸ bulk TIL cells. In some embodiments, this priming first expansion occurs for a period of about 7 to 8 days, resulting in a bulk TIL population, generally about 1×10⁸ bulk TIL cells. In some embodiments, this priming first expansion occurs for a period of about 7 days, resulting in a bulk TIL population, generally about 1×10⁸ bulk TIL cells. In some embodiments, this priming first expansion occurs for a period of about 8 days, resulting in a bulk TIL population, generally about 1×10⁸ bulk TIL cells.

In a preferred embodiment, expansion of TILs may be performed using a priming first expansion step (for example such as those described in Step B of FIG. 8 (in particular, e.g., FIG. 8B and/or FIG. 8C), which can include processes referred to as pre-REP or priming REP and which contains feeder cells from Day 0 and/or from culture initiation) as described below and herein, followed by a rapid second expansion (Step D, including processes referred to as rapid expansion protocol (REP) steps) as described below under Step D and herein, followed by optional cryopreservation, and followed by a second Step D (including processes referred to as restimulation REP steps) as described below and herein. The TILs obtained from this process may be optionally characterized for phenotypic characteristics and metabolic parameters as described herein. In some embodiments, the tumor fragment is between about 1 mm³ and 10 mm³.

In some embodiments, the first expansion culture medium is referred to as “CM”, an abbreviation for culture media. In some embodiments, CM for Step B consists of RPMI 1640 with GlutaMAX, supplemented with 10% human AB serum, 25 mM Hepes, and 10 mg/mL gentamicin.

In some embodiments, there are less than or equal to 240 tumor fragments. In some embodiments, there are less than or equal to 240 tumor fragments placed in less than or equal to 4 containers. In some embodiments, the containers are GREX100 MCS flasks. In some embodiments, less than or equal to 60 tumor fragments are placed in 1 container. In some embodiments, each container comprises less than or equal to 500 mL of media per container. In some embodiments, the media comprises IL-2. In some embodiments, the media comprises 6000 IU/mL of IL-2. In some embodiments, the media comprises antigen-presenting feeder cells (also referred to herein as “antigen-presenting cells”). In some embodiments, the media comprises 2.5×10⁸ antigen-presenting feeder cells per container. In some embodiments, the media comprises OKT-3. In some embodiments, the media comprises 30 ng/mL of OKT-3 per container. In some embodiments, the container is a GREX100 MCS flask. In some embodiments, the media comprises 6000 IU/mL of IL-2, 30 ng of OKT-3, and 2.5×10⁸ antigen-presenting feeder cells. In some embodiments, the media comprises 6000 IU/mL of IL-2, 30 ng/mL of OKT-3, and 2.5×10⁸ antigen-presenting feeder cells per container.

After preparation of the tumor fragments, the resulting cells (i.e., fragments which is a primary cell population) are cultured in media containing IL-2, antigen-presenting feeder cells and OKT-3 under conditions that favor the growth of TILs over tumor and other cells and which allow for TIL priming and accelerated growth from initiation of the culture on Day 0. In some embodiments, the tumor digests and/or tumor fragments are incubated in with 6000 IU/mL of IL-2, as well as antigen-presenting feeder cells and OKT-3. This primary cell population is cultured for a period of days, generally from 1 to 8 days, resulting in a bulk TIL population, generally about 1×10⁸ bulk TIL cells. In some embodiments, the growth media during the priming first expansion comprises IL-2 or a variant thereof, as well as antigen-presenting feeder cells and OKT-3. In some embodiments, this primary cell population is cultured for a period of days, generally from 1 to 7 days, resulting in a bulk TIL population, generally about 1×10⁸ bulk TIL cells. In some embodiments, the growth media during the priming first expansion comprises IL-2 or a variant thereof, as well as antigen-presenting feeder cells and OKT-3. In some embodiments, the IL-2 is recombinant human IL-2 (rhIL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the IL-2 stock solution is prepare as described in Example C. In some embodiments, the priming first expansion culture media comprises about 10,000 IU/mL of IL-2, about 9,000 IU/mL of IL-2, about 8,000 IU/mL of IL-2, about 7,000 IU/mL of IL-2, about 6000 IU/mL of IL-2 or about 5,000 IU/mL of IL-2. In some embodiments, the priming first expansion culture media comprises about 9,000 IU/mL of IL-2 to about 5,000 IU/mL of IL-2. In some embodiments, the priming first expansion culture media comprises about 8,000 IU/mL of IL-2 to about 6,000 IU/mL of IL-2. In some embodiments, the priming first expansion culture media comprises about 7,000 IU/mL of IL-2 to about 6,000 IU/mL of IL-2. In some embodiments, the priming first expansion culture media comprises about 6,000 IU/mL of IL-2. In an embodiment, the cell culture medium further comprises IL-2. In some embodiments, the priming first expansion cell culture medium comprises about 3000 IU/mL of IL-2. In an embodiment, the priming first expansion cell culture medium further comprises IL-2. In a preferred embodiment, the priming first expansion cell culture medium comprises about 3000 IU/mL of IL-2. In an embodiment, the priming first expansion cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the priming first expansion cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or about 8000 IU/mL of IL-2.

In some embodiments, priming first expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the priming first expansion culture media comprises about 500 IU/mL of IL-15 to about 100 IU/mL of IL-15. In some embodiments, the priming first expansion culture media comprises about 400 IU/mL of IL-15 to about 100 IU/mL of IL-15. In some embodiments, the priming first expansion culture media comprises about 300 IU/mL of IL-15 to about 100 IU/mL of IL-15. In some embodiments, the priming first expansion culture media comprises about 200 IU/mL of IL-15. In some embodiments, the priming first expansion cell culture medium comprises about 180 IU/mL of IL-15. In an embodiment, the priming first expansion cell culture medium further comprises IL-15. In a preferred embodiment, the priming first expansion cell culture medium comprises about 180 IU/mL of IL-15.

In some embodiments, priming first expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the priming first expansion culture media comprises about 20 IU/mL of IL-21 to about 0.5 IU/mL of IL-21. In some embodiments, the priming first expansion culture media comprises about 15 IU/mL of IL-21 to about 0.5 IU/mL of IL-21. In some embodiments, the priming first expansion culture media comprises about 12 IU/mL of IL-21 to about 0.5 IU/mL of IL-21. In some embodiments, the priming first expansion culture media comprises about 10 IU/mL of IL-21 to about 0.5 IU/mL of IL-21. In some embodiments, the priming first expansion culture media comprises about 5 IU/mL of IL-21 to about 1 IU/mL of IL-21. In some embodiments, the priming first expansion culture media comprises about 2 IU/mL of IL-21. In some embodiments, the priming first expansion cell culture medium comprises about 1 IU/mL of IL-21. In some embodiments, the priming first expansion cell culture medium comprises about 0.5 IU/mL of IL-21. In an embodiment, the cell culture medium further comprises IL-21. In a preferred embodiment, the priming first expansion cell culture medium comprises about 1 IU/mL of IL-21.

In an embodiment, the priming first expansion cell culture medium comprises OKT-3 antibody. In some embodiments, the priming first expansion cell culture medium comprises about 30 ng/mL of OKT-3 antibody. In an embodiment, the priming first expansion cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In an embodiment, the cell culture medium comprises between 15 ng/mL and 30 ng/mL of OKT-3 antibody. In an embodiment, the cell culture medium comprises 30 ng/mL of OKT-3 antibody. In some embodiments, the OKT-3 antibody is muromonab.

In some embodiments, the priming first expansion cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 pg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the priming first expansion cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist. In some embodiments, in addition to one or more TNFRSF agonists, the priming first expansion cell culture medium further comprises IL-2 at an initial concentration of about 6000 IU/mL and OKT-3 antibody at an initial concentration of about 30 ng/mL, and wherein the one or more TNFRSF agonists comprises a 4-1BB agonist.

In some embodiments, the priming first expansion culture medium is referred to as “CM”, an abbreviation for culture media. In some embodiments, it is referred to as CM1 (culture medium 1). In some embodiments, CM consists of RPMI 1640 with GlutaMAX, supplemented with 10% human AB serum, 25 mM Hepes, and 10 mg/mL gentamicin. In some embodiments, the CM is the CM1 described in the Examples. In some embodiments, the priming first expansion occurs in an initial cell culture medium or a first cell culture medium. In some embodiments, the priming first expansion culture medium or the initial cell culture medium or the first cell culture medium comprises IL-2, OKT-3 and antigen-presenting feeder cells (also referred to herein as feeder cells).

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium, CTS™ OpTmizer™ T-Cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™ OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CST™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific). In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM. In some embodiments, the final concentration of 2-mercaptoethanol in the media is 55 μM.

In some embodiments, the defined media described in International PCT Publication No. WO/1998/030679, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the basal cell media is selected from the group consisting of Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1× Medium” in Table 4 below. In other embodiments, the non-trace element moiety ingredients in the defined medium are present in the final concentrations listed in the column under the heading “A Preferred Embodiment of the 1× Medium” in Table 4 below. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 4 below.

TABLE 4
Concentrations of Non-Trace Element Moiety Ingredients

| | | | A preferred |
|---|---|---|---|
| | A preferred | Concentration | embodiment |
| | embodiment | range in 1X | in 1X |
| | in supplement | medium | medium |
| | (mg/L) | (mg/L) | (mg/L) |
| Ingredient | (About) | (About) | (About) |
| Glycine | 150 | 5-200 | 53 |
| L-Histidine | 940 | 5-250 | 183 |
| L-Isoleucine | 3400 | 5-300 | 615 |
| L-Methionine | 90 | 5-200 | 44 |
| L-Phenylalanine | 1800 | 5-400 | 336 |
| L-Proline | 4000 | 1-1000 | 600 |
| L-Hydroxyproline | 100 | 1-45 | 15 |
| L-Serine | 800 | 1-250 | 162 |
| L-Threonine | 2200 | 10-500 | 425 |
| L-Tryptophan | 440 | 2-110 | 82 |
| L-Tyrosine | 77 | 3-175 | 84 |
| L-Valine | 2400 | 5-500 | 454 |
| Thiamine | 33 | 1-20 | 9 |
| Reduced Glutathione | 10 | 1-20 | 1.5 |
| Ascorbic Acid-2-PO₄ | 330 | 1-200 | 50 |
| (Mg Salt) | | | |
| Transferrin (iron | 55 | 1-50 | 8 |
| saturated) | | | |
| Insulin | 100 | 1-100 | 10 |
| Sodium Selenite | 0.07 | 0.000001-0.0001 | 0.00001 |
| AlbuMAX ®I | 83,000 | 5000-50,000 | 12,500 |

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 μM).

In some embodiments, the defined media described in Smith, et al., Clin. Transl. Immunology, 2015, 4(1), e31, the disclosures of which are incorporated by reference herein, are useful in the present invention. Briefly, RPMI or CTS™ OpTmizer™ was used as the basal cell medium, and supplemented with either 0, 2%, 5%, or 10% CTS™ Immune Cell Serum Replacement.

In an embodiment, the cell medium in the first and/or second gas permeable container is unfiltered. The use of unfiltered cell medium may simplify the procedures necessary to expand the number of cells. In an embodiment, the cell medium in the first and/or second gas permeable container lacks beta-mercaptoethanol (BME or BME; also known as 2-mercaptoethanol, CAS 60-24-2).

In some embodiments, the priming first expansion (including processes such as for example those described in Step B of FIG. 1 (in particular, e.g., FIG. 1B and/or FIG. 8C), which can include those sometimes referred to as the pre-REP or priming REP) process is 1 to 8 days, as discussed in the examples and figures. In some embodiments, the priming first expansion (including processes such as for example those described in Step B of FIG. 1 (in particular, e.g., FIG. 1B and/or FIG. 8C), which can include those sometimes referred to as the pre-REP or priming REP) process is 2 to 8 days. In some embodiments, the priming first expansion (including processes such as for example those described in Step B of FIG. 1 (in particular, e.g., FIG. 1B and/or FIG. 8C), which can include those sometimes referred to as the pre-REP or priming REP) process is 3 to 8 days. In some embodiments, the priming first expansion (including processes such as for example those described in Step B of FIG. 1 (in particular, e.g., FIG. 8B and/or FIG. 8C), which can include those sometimes referred to as the pre-REP or priming REP) process is 4 to 8 days, as discussed in the examples and figures. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 1 to 7 days, as discussed in the examples and figures. In some embodiments, the priming first expansion (including processes such as for example those described in Step B of FIG. 1 (in particular, e.g., FIG. 1B and/or FIG. 8C), which can include those sometimes referred to as the pre-REP or priming REP) process is 2 to 8 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 2 to 7 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 3 to 8 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 3 to 7 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 4 to 8 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 4 to 7 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 5 to 8 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 5 to 7 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 6 to 8 days. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 6 to 7 days. In some embodiments, the priming first expansion (including processes such as for example those provided in Step B of FIG. 1 (in particular, e.g., FIG. 8B and/or FIG. 8C), which can include those sometimes referred to as the pre-REP or priming REP) process is 7 to 8 days. In some embodiments, the priming first expansion (including processes such as for example those provided in Step B of FIG. 8 (in particular, e.g., FIG. 8B and/or FIG. 8C), which can include those sometimes referred to as the pre-REP or priming REP) process is 8 days. In some embodiments, the priming first expansion (including processes such as for example those provided in Step B of FIG. 8 (in particular, e.g., FIG. 8B and/or FIG. 8C), which can include those sometimes referred to as the pre-REP or priming REP) process is 7 days.

In some embodiments, the priming first TIL expansion can proceed for 1 days to 8 days from when fragmentation occurs and/or when the first priming expansion step is initiated. In some embodiments, the priming first TIL expansion can proceed for 1 days to 7 days from when fragmentation occurs and/or when the first priming expansion step is initiated. In some embodiments, the priming first TIL expansion can proceed for 2 days to 8 days from when fragmentation occurs and/or when the first priming expansion step is initiated. In some embodiments, the priming first TIL expansion can proceed for 2 days to 7 days from when fragmentation occurs and/or when the first priming expansion step is initiated. In some embodiments, the priming first TIL expansion can proceed for 3 days to 8 days from when fragmentation occurs and/or when the first priming expansion step is initiated. In some embodiments, the priming first TIL expansion can proceed for 3 days to 7 days from when fragmentation occurs and/or when the first priming expansion step is initiated. In some embodiments, the priming first TIL expansion can proceed for 4 days to 8 days from when fragmentation occurs and/or when the first priming expansion step is initiated. In some embodiments, the priming first TIL expansion can proceed for 4 days to 7 days from when fragmentation occurs and/or when the first priming expansion step is initiated. In some embodiments, the priming first TIL expansion can proceed for 5 days to 8 days from when fragmentation occurs and/or when the first priming expansion step is initiated. In some embodiments, the priming first TIL expansion can proceed for 5 days to 7 days from when fragmentation occurs and/or when the first priming expansion step is initiated. In some embodiments, the priming first TIL expansion can proceed for 6 days to 8 days from when fragmentation occurs and/or when the first priming expansion step is initiated. In some embodiments, the priming first TIL expansion can proceed for 6 days to 7 days from when fragmentation occurs and/or when the first priming expansion step is initiated. In some embodiments, the priming first TIL expansion can proceed for 7 to 8 days from when fragmentation occurs and/or when the first priming expansion step is initiated. In some embodiments, the priming first TIL expansion can proceed for 8 days from when fragmentation occurs and/or when the first priming expansion step is initiated. In some embodiments, the priming first TIL expansion can proceed for 7 days from when fragmentation occurs and/or when the first priming expansion step is initiated.

In some embodiments, the priming first expansion of the TILs can proceed for 1 day, 2 days, 3 days, 4 days, 5 days, 6 days, 7 days, or 8 days. In some embodiments, the first TIL expansion can proceed for 1 day to 8 days. In some embodiments, the first TIL expansion can proceed for 1 day to 7 days. In some embodiments, the first TIL expansion can proceed for 2 days to 8 days. In some embodiments, the first TIL expansion can proceed for 2 days to 7 days. In some embodiments, the first TIL expansion can proceed for 3 days to 8 days. In some embodiments, the first TIL expansion can proceed for 3 days to 7 days. In some embodiments, the first TIL expansion can proceed for 4 days to 8 days. In some embodiments, the first TIL expansion can proceed for 4 days to 7 days. In some embodiments, the first TIL expansion can proceed for 5 days to 8 days. In some embodiments, the first TIL expansion can proceed for 5 days to 7 days. In some embodiments, the first TIL expansion can proceed for 6 days to 8 days. In some embodiments, the first TIL expansion can proceed for 6 days to 7 days. In some embodiments, the first TIL expansion can proceed for 7 to 8 days. In some embodiments, the first TIL expansion can proceed for 8 days. In some embodiments, the first TIL expansion can proceed for 7 days.

In some embodiments, a combination of IL-2, IL-7, IL-15, and/or IL-21 are employed as a combination during the priming first expansion. In some embodiments, IL-2, IL-7, IL-15, and/or IL-21 as well as any combinations thereof can be included during the priming first expansion, including, for example during Step B processes according to FIG. 8 (in particular, e.g., FIG. 8B and/or FIG. 8C), as well as described herein. In some embodiments, a combination of IL-2, IL-15, and IL-21 are employed as a combination during the priming first expansion. In some embodiments, IL-2, IL-15, and IL-21 as well as any combinations thereof can be included during Step B processes according to FIG. 8 (in particular, e.g., FIG. 8B and/or FIG. 8C) and as described herein.

In some embodiments, the priming first expansion, for example, Step B according to FIG. 8 (in particular, e.g., FIG. 8B and/or FIG. 8C), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-10 or a G-REX-100. In some embodiments, the bioreactor employed is a G-REX-100. In some embodiments, the bioreactor employed is a G-REX-10.

### 1. Feeder Cells and Antigen Presenting Cells

In an embodiment, the priming first expansion procedures described herein (for example including expansion such as those described in Step B from FIG. 1 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during days 4-8. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during days 4-7. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during days 5-8. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during days 5-7. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during days 6-8. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during days 6-7. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during day 7 or 8. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during day 7. In an embodiment, the priming first expansion procedures described herein (for example including expansion such as those described in Step B from FIG. 8 (in particular, e.g., FIG. 8B and/or FIG. 8C), as well as those referred to as pre-REP or priming REP) does not require feeder cells (also referred to herein as “antigen-presenting cells”) at the initiation of the TIL expansion, but rather are added during the priming first expansion at any time during day 8

In an embodiment, the priming first expansion procedures described herein (for example including expansion such as those described in Step B from FIG. 8 (in particular, e.g., FIG. 8B), as well as those referred to as pre-REP or priming REP) require feeder cells (also referred to herein as “antigen-presenting cells”) at the initiation of the TIL expansion and during the priming first expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In some embodiments, 2.5×10⁸ feeder cells are used during the priming first expansion. In some embodiments, 2.5×10⁸ feeder cells per container are used during the priming first expansion. In some embodiments, 2.5×10⁸ feeder cells per GREX-10 are used during the priming first expansion. In some embodiments, 2.5×10⁸ feeder cells per GREX-100 are used during the priming first expansion.

In general, the allogeneic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and acceptable for use in the TIL expansion procedures described herein if the total number of viable cells on day 14 is less than the initial viable cell number put into culture on day 0 of the priming first expansion.

In some embodiments, PBMCs are considered replication incompetent and acceptable for use in the TIL expansion procedures described herein if the total number of viable cells, cultured in the presence of OKT3 and IL-2, on day 7 have not increased from the initial viable cell number put into culture on day 0 of the priming first expansion. In some embodiments, the PBMCs are cultured in the presence of 30 ng/mL OKT3 antibody and 3000 IU/mL IL-2. In some embodiments, the PBMCs are cultured in the presence of 30 ng/mL OKT3 antibody and 6000 IU/mL IL-2.

In some embodiments, PBMCs are considered replication incompetent and acceptable for use in the TIL expansion procedures described herein if the total number of viable cells, cultured in the presence of OKT3 and IL-2, on day 7 have not increased from the initial viable cell number put into culture on day 0 of the priming first expansion. In some embodiments, the PBMCs are cultured in the presence of 5-60 ng/mL OKT3 antibody and 1000-6000 IU/mL IL-2. In some embodiments, the PBMCs are cultured in the presence of 10-50 ng/mL OKT3 antibody and 2000-5000 IU/mL IL-2. In some embodiments, the PBMCs are cultured in the presence of 20-40 ng/mL OKT3 antibody and 2000-4000 IU/mL IL-2. In some embodiments, the PBMCs are cultured in the presence of 25-35 ng/mL OKT3 antibody and 2500-3500 IU/mL IL-2. In some embodiments, the PBMCs are cultured in the presence of 30 ng/mL OKT3 antibody and 6000 IU/mL IL-2. In some embodiments, the PBMCs are cultured in the presence of 15 ng/mL OKT3 antibody and 3000 IU/mL IL-2. In some embodiments, the PBMCs are cultured in the presence of 15 ng/mL OKT3 antibody and 6000 IU/mL IL-2.

In some embodiments, the antigen-presenting feeder cells are PBMCs. In some embodiments, the antigen-presenting feeder cells are artificial antigen-presenting feeder cells. In an embodiment, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to antigen-presenting feeder cells in the second expansion is between 1 to 50 and 1 to 300. In an embodiment, the ratio of TILs to antigen-presenting feeder cells in the second expansion is between 1 to 100 and 1 to 200.

In an embodiment, the priming first expansion procedures described herein require a ratio of about 2.5×10⁸ feeder cells to about 100×10⁶ TILs. In another embodiment, the priming first expansion procedures described herein require a ratio of about 2.5×10⁸ feeder cells to about 50×10⁶ TILs. In yet another embodiment, the priming first expansion described herein require about 2.5×10⁸ feeder cells to about 25×10⁶ TILs. In yet another embodiment, the priming first expansion described herein require about 2.5×10⁸ feeder cells. In yet another embodiment, the priming first expansion requires one-fourth, one-third, five-twelfths, or one-half of the number of feeder cells used in the rapid second expansion.

In some embodiments, the media in the priming first expansion comprises IL-2. In some embodiments, the media in the priming first expansion comprises 6000 IU/mL of IL-2. In some embodiments, the media in the priming first expansion comprises antigen-presenting feeder cells. In some embodiments, the media in the priming first expansion comprises 2.5×10⁸ antigen-presenting feeder cells per container. In some embodiments, the media in the priming first expansion comprises OKT-3. In some embodiments, the media comprises 30 ng of OKT-3 per container. In some embodiments, the container is a GREX100 MCS flask. In some embodiments, the media comprises 6000 IU/mL of LL-2, 30 ng/mL of OKT-3, and 2.5×10⁸ antigen-presenting feeder cells. In some embodiments, the media comprises 6000 IU/mL of IL-2, 30 ng/mL of OKT-3, and 2.5×10⁸ antigen-presenting feeder cells per container. In some embodiments, the media comprises 500 mL of culture medium and 15 μg of OKT-3 per 2.5×10⁸ antigen-presenting feeder cells per container. In some embodiments, the media comprises 500 mL of culture medium and 15 μg of OKT-3 per container. In some embodiments, the container is a GREX100 MCS flask. In some embodiments, the media comprises 500 mL of culture medium, 6000 IU/mL of IL-2, 30 ng/mL of OKT-3, and 2.5×10⁸ antigen-presenting feeder cells. In some embodiments, the media comprises 500 mL of culture medium, 6000 IU/mL of IL-2, 15 μg of OKT-3, and 2.5×10⁸ antigen-presenting feeder cells per container. In some embodiments, the media comprises 500 mL of culture medium and 15 μg of OKT-3 per 2.5×10⁸ antigen-presenting feeder cells per container.

In an embodiment, the priming first expansion procedures described herein require an excess of feeder cells over TILs during the second expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In an embodiment, artificial antigen-presenting (aAPC) cells are used in place of PBMCs.

In general, the allogeneic PBMCs are inactivated, either via irradiation or heat treatment, and used in the TIL expansion procedures described herein, including the exemplary procedures described in the figures and examples.

In an embodiment, artificial antigen presenting cells are used in the priming first expansion as a replacement for, or in combination with, PBMCs.

### 2. Cytokines and Other Additives

The expansion methods described herein generally use culture media with high doses of a cytokine, in particular IL-2, as is known in the art.

Alternatively, using combinations of cytokines for the priming first expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21, and IL-2, IL-15 and IL-21, with the latter finding particular use in many embodiments. The use of combinations of cytokines specifically favors the generation of lymphocytes, and in particular T-cells as described therein.

In an embodiment, Step B may also include the addition of OKT-3 antibody or muromonab to the culture media, as described elsewhere herein. In an embodiment, Step B may also include the addition of a 4-1BB agonist to the culture media, as described elsewhere herein. In an embodiment, Step B may also include the addition of an OX-40 agonist to the culture media, as described elsewhere herein. In addition, additives such as peroxisome proliferator-activated receptor gamma coactivator I-alpha agonists, including proliferator-activated receptor (PPAR)-gamma agonists such as a thiazolidinedione compound, may be used in the culture media during Step B, as described in U.S. Patent Application Publication No. US 2019/0307796 A1, the disclosure of which is incorporated by reference herein.

### C. STEP C: Priming First Expansion to Rapid Second Expansion Transition

In some cases, the bulk TIL population obtained from the priming first expansion (which can include expansions sometimes referred to as pre-REP), including, for example the TIL population obtained from for example, Step B as indicated in FIG. 8 (in particular, e.g., FIG. 8B and/or FIG. 8C), can be subjected to a rapid second expansion (which can include expansions sometimes referred to as Rapid Expansion Protocol (REP)) and then cryopreserved as discussed below. Similarly, in the case where genetically modified TILs will be used in therapy, the expanded TIL population from the priming first expansion or the expanded TIL population from the rapid second expansion can be subjected to genetic modifications for suitable treatments prior to the expansion step or after the priming first expansion and prior to the rapid second expansion.

In some embodiments, the TILs obtained from the priming first expansion (for example, from Step B as indicated in FIG. 1 (in particular, e.g., FIG. 1B and/or FIG. 8C)) are stored until phenotyped for selection. In some embodiments, the TILs obtained from the priming first expansion (for example, from Step B as indicated in FIG. 1 (in particular, e.g., FIG. 1B and/or FIG. 8C)) are not stored and proceed directly to the rapid second expansion. In some embodiments, the TILs obtained from the priming first expansion are not cryopreserved after the priming first expansion and prior to the rapid second expansion. In some embodiments, the transition from the priming first expansion to the second expansion occurs at about 2 days, 3 days, 4, days, 5 days, 6 days, 7 days, or 8 days from when tumor fragmentation occurs and/or when the first priming expansion step is initiated. In some embodiments, the transition from the priming first expansion to the rapid second expansion occurs at about 3 days to 7 days from when fragmentation occurs and/or when the first priming expansion step is initiated. In some embodiments, the transition from the priming first expansion to the rapid second expansion occurs at about 3 days to 8 days from when fragmentation occurs and/or when the first priming expansion step is initiated. In some embodiments, the transition from the priming first expansion to the second expansion occurs at about 4 days to 7 days from when fragmentation occurs and/or when the first priming expansion step is initiated. In some embodiments, the transition from the priming first expansion to the second expansion occurs at about 4 days to 8 days from when fragmentation occurs and/or when the first priming expansion step is initiated. In some embodiments, the transition from the priming first expansion to the second expansion occurs at about 5 days to 7 days from when fragmentation occurs and/or when the first priming expansion step is initiated. In some embodiments, the transition from the priming first expansion to the second expansion occurs at about 5 days to 8 days from when fragmentation occurs and/or when the first priming expansion step is initiated. In some embodiments, the transition from the priming first expansion to the second expansion occurs at about 6 days to 7 days from when fragmentation occurs and/or when the first priming expansion step is initiated. In some embodiments, the transition from the priming first expansion to the second expansion occurs at about 6 days to 8 days from when fragmentation occurs and/or when the first priming expansion step is initiated. In some embodiments, the transition from the priming first expansion to the second expansion occurs at about 7 days to 8 days from when fragmentation occurs and/or when the first priming expansion step is initiated. In some embodiments, the transition from the priming first expansion to the second expansion occurs at about 7 days from when fragmentation occurs and/or when the first priming expansion step is initiated. In some embodiments, the transition from the priming first expansion to the second expansion occurs at about 8 days from when fragmentation occurs and/or when the first priming expansion step is initiated.

In some embodiments, the transition from the priming first expansion to the rapid second expansion occurs at 1 day, 2 days, 3 days, 4 days, 5 days, 6 days, 7 days, or 8 days from when fragmentation occurs and/or when the first priming expansion step is initiated. In some embodiments, the transition from the priming first expansion to the rapid second expansion occurs 1 day to 7 days from when fragmentation occurs and/or when the first priming expansion step is initiated. In some embodiments, the transition from the priming first expansion to the rapid second expansion occurs 1 day to 8 days from when fragmentation occurs and/or when the first priming expansion step is initiated. In some embodiments, the transition from the priming first expansion to the second expansion occurs 2 days to 7 days from when fragmentation occurs and/or when the first priming expansion step is initiated. In some embodiments, the transition from the priming first expansion to the second expansion occurs 2 days to 8 days from when fragmentation occurs and/or when the first priming expansion step is initiated. In some embodiments, the transition from the priming first expansion to the second expansion occurs 3 days to 7 days from when fragmentation occurs and/or when the first priming expansion step is initiated. In some embodiments, the transition from the priming first expansion to the second expansion occurs 3 days to 8 days from when fragmentation occurs and/or when the first priming expansion step is initiated. In some embodiments, the transition from the priming first expansion to the rapid second expansion occurs 4 days to 7 days from when fragmentation occurs and/or when the first priming expansion step is initiated. In some embodiments, the transition from the priming first expansion to the rapid second expansion occurs 4 days to 8 days from when fragmentation occurs and/or when the first priming expansion step is initiated. In some embodiments, the transition from the priming first expansion to the rapid second expansion occurs 5 days to 7 days from when fragmentation occurs and/or when the first priming expansion step is initiated. In some embodiments, the transition from the priming first expansion to the rapid second expansion occurs 5 days to 8 days from when fragmentation occurs and/or when the first priming expansion step is initiated. In some embodiments, the transition from the priming first expansion to the rapid second expansion occurs 6 days to 7 days from when fragmentation occurs and/or when the first priming expansion step is initiated. In some embodiments, the transition from the priming first expansion to the rapid second expansion occurs 6 days to 8 days from when fragmentation occurs and/or when the first priming expansion step is initiated. In some embodiments, the transition from the priming first expansion to the rapid second expansion occurs 7 days to 8 days from when fragmentation occurs and/or when the first priming expansion step is initiated. In some embodiments, the transition from the priming first expansion to the rapid second expansion occurs 7 days from when fragmentation occurs and/or when the first priming expansion step is initiated. In some embodiments, the transition from the priming first expansion to the rapid second expansion occurs 8 days from when fragmentation occurs and/or when the first priming expansion step is initiated.

In some embodiments, the TILs are not stored after the primary first expansion and prior to the rapid second expansion, and the TILs proceed directly to the rapid second expansion (for example, in some embodiments, there is no storage during the transition from Step B to Step D as shown in FIG. 8 (in particular, e.g., FIG. 8B and/or FIG. 8C)). In some embodiments, the transition occurs in closed system, as described herein. In some embodiments, the TILs from the priming first expansion, the second population of TILs, proceeds directly into the rapid second expansion with no transition period.

In some embodiments, the transition from the priming first expansion to the rapid second expansion, for example, Step C according to FIG. 8 (in particular, e.g., FIG. 8B and/or FIG. 8C), is performed in a closed system bioreactor. In some embodiments, a closed system is employed for the TIL expansion, as described herein. In some embodiments, a single bioreactor is employed. In some embodiments, the single bioreactor employed is for example a GREX-10 or a GREX-100. In some embodiments, the closed system bioreactor is a single bioreactor. In some embodiments, the transition from the priming first expansion to the rapid second expansion involves a scale-up in container size. In some embodiments, the priming first expansion is performed in a smaller container than the rapid second expansion. In some embodiments, the priming first expansion is performed in a GREX-100 and the rapid second expansion is performed in a GREX-500.

### D. STEP D: Rapid Second Expansion

In some embodiments, the TIL cell population is further expanded in number after harvest and the priming first expansion, after Step A and Step B, and the transition referred to as Step C, as indicated in FIG. 8 (in particular, e.g., FIG. 8B and/or FIG. 8C)). This further expansion is referred to herein as the rapid second expansion or a rapid expansion, which can include expansion processes generally referred to in the art as a rapid expansion process (Rapid Expansion Protocol or REP; as well as processes as indicated in Step D of FIG. 8 (in particular, e.g., FIG. 8B)). The rapid second expansion is generally accomplished using a culture media comprising a number of components, including feeder cells, a cytokine source, and an anti-CD3 antibody, in a gas-permeable container. In some embodiments, 1 day, 2 days, 3 days, or 4 days after initiation of the rapid second expansion (i.e., at days 8, 9, 10, or 11 of the overall Gen 3 process), the TILs are transferred to a larger volume container.

In some embodiments, the rapid second expansion (which can include expansions sometimes referred to as REP; as well as processes as indicated in Step D of FIG. 1 (in particular, e.g., FIG. 1B and/or FIG. 8C)) of TIL can be performed using any TIL flasks or containers known by those of skill in the art. In some embodiments, the second TIL expansion can proceed for 1 day, 2 days, 3 days, 4, days, 5 days, 6 days, 7 days, 8 days, 9 days or 10 days after initiation of the rapid second expansion. In some embodiments, the second TIL expansion can proceed for about 1 days to about 9 days after initiation of the rapid second expansion. In some embodiments, the second TIL expansion can proceed for about 1 days to about 10 days after initiation of the rapid second expansion. In some embodiments, the second TIL expansion can proceed for about 2 days to about 9 days after initiation of the rapid second expansion. In some embodiments, the second TIL expansion can proceed for about 2 days to about 10 days after initiation of the rapid second expansion. In some embodiments, the second TIL expansion can proceed for about 3 days to about 9 days after initiation of the rapid second expansion. In some embodiments, the second TIL expansion can proceed for about 3 days to about 10 days after initiation of the rapid second expansion. In some embodiments, the second TIL expansion can proceed for about 4 days to about 9 days after initiation of the rapid second expansion. In some embodiments, the second TIL expansion can proceed for about 4 days to about 10 days after initiation of the rapid second expansion. In some embodiments, the second TIL expansion can proceed for about 5 days to about 9 days after initiation of the rapid second expansion. In some embodiments, the second TIL expansion can proceed for about 5 days to about 10 days after initiation of the rapid second expansion. In some embodiments, the second TIL expansion can proceed for about 6 days to about 9 days after initiation of the rapid second expansion. In some embodiments, the second TIL expansion can proceed for about 6 days to about 10 days after initiation of the rapid second expansion. In some embodiments, the second TIL expansion can proceed for about 7 days to about 9 days after initiation of the rapid second expansion. In some embodiments, the second TIL expansion can proceed for about 7 days to about 10 days after initiation of the rapid second expansion. In some embodiments, the second TIL expansion can proceed for about 8 days to about 9 days after initiation of the rapid second expansion. In some embodiments, the second TIL expansion can proceed for about 8 days to about 10 days after initiation of the rapid second expansion. In some embodiments, the second TIL expansion can proceed for about 9 days to about 10 days after initiation of the rapid second expansion. In some embodiments, the second TIL expansion can proceed for about 1 day after initiation of the rapid second expansion. In some embodiments, the second TIL expansion can proceed for about 2 days after initiation of the rapid second expansion. In some embodiments, the second TIL expansion can proceed for about 3 days after initiation of the rapid second expansion. In some embodiments, the second TIL expansion can proceed for about 4 days after initiation of the rapid second expansion. In some embodiments, the second TIL expansion can proceed for about 5 days after initiation of the rapid second expansion. In some embodiments, the second TIL expansion can proceed for about 6 days after initiation of the rapid second expansion. In some embodiments, the second TIL expansion can proceed for about 7 days after initiation of the rapid second expansion. In some embodiments, the second TIL expansion can proceed for about 8 days after initiation of the rapid second expansion. In some embodiments, the second TIL expansion can proceed for about 9 days after initiation of the rapid second expansion. In some embodiments, the second TIL expansion can proceed for about 10 days after initiation of the rapid second expansion.

In an embodiment, the rapid second expansion can be performed in a gas permeable container using the methods of the present disclosure (including, for example, expansions referred to as REP; as well as processes as indicated in Step D of FIG. 8 (in particular, e.g., FIG. 8B and/or FIG. 8C). In some embodiments, the TILs are expanded in the rapid second expansion in the presence of IL-2, OKT-3, and feeder cells (also referred herein as “antigen-presenting cells”). In some embodiments, the TILs are expanded in the rapid second expansion in the presence of IL-2, OKT-3, and feeder cells, wherein the feeder cells are added to a final concentration that is twice, 2.4 times, 2.5 times, 3 times, 3.5 times or 4 times the concentration of feeder cells present in the priming first expansion. For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/mL of OKT3, a mouse monoclonal anti-CD3 antibody (commercially available from Ortho-McNeil, Raritan, NJ or Miltenyi Biotech, Auburn, CA) or UHCT-1 (commercially available from BioLegend, San Diego, CA, USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In an embodiment, the cell culture medium further comprises IL-2. In some embodiments, the cell culture medium comprises about 3000 IU/mL of IL-2. In an embodiment, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In an embodiment, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In an embodiment, the cell culture medium comprises OKT-3 antibody. In some embodiments, the cell culture medium comprises about 30 ng/mL of OKT-3 antibody. In an embodiment, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 pg/mL of OKT-3 antibody. In an embodiment, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In an embodiment, the cell culture medium comprises between 15 ng/mL and 30 ng/mL of OKT-3 antibody. In an embodiment, the cell culture medium comprises between 30 ng/mL and 60 ng/mL of OKT-3 antibody. In an embodiment, the cell culture medium comprises about 30 ng/mL OKT-3. In an embodiment, the cell culture medium comprises about 60 ng/mL OKT-3. In some embodiments, the OKT-3 antibody is muromonab.

In some embodiments, the media in the rapid second expansion comprises IL-2. In some embodiments, the media comprises 6000 IU/mL of IL-2. In some embodiments, the media in the rapid second expansion comprises antigen-presenting feeder cells. In some embodiments, the media in the rapid second expansion comprises 7.5×10⁸ antigen-presenting feeder cells per container. In some embodiments, the media in the rapid second expansion comprises OKT-3. In some embodiments, the in the rapid second expansion media comprises 500 mL of culture medium and 30 μg of OKT-3 per container. In some embodiments, the container is a GREX100 MCS flask. In some embodiments, the in the rapid second expansion media comprises 6000 IU/mL of IL-2, 60 ng/mL of OKT-3, and 7.5×10⁸ antigen-presenting feeder cells. In some embodiments, the media comprises 500 mL of culture medium and 6000 IU/mL of IL-2, 30 μg of OKT-3, and 7.5×10⁸ antigen-presenting feeder cells per container.

In some embodiments, the media in the rapid second expansion comprises IL-2. In some embodiments, the media comprises 6000 IU/mL of IL-2. In some embodiments, the media in the rapid second expansion comprises antigen-presenting feeder cells. In some embodiments, the media comprises between 5×10⁸ and 7.5×10⁸ antigen-presenting feeder cells per container. In some embodiments, the media in the rapid second expansion comprises OKT-3. In some embodiments, the media in the rapid second expansion comprises 500 mL of culture medium and 30 μg of OKT-3 per container. In some embodiments, the container is a GREX100 MCS flask. In some embodiments, the media in the rapid second expansion comprises 6000 IU/mL of IL-2, 60 ng/mL of OKT-3, and between 5×10⁸ and 7.5×10⁸ antigen-presenting feeder cells. In some embodiments, the media in the rapid second expansion comprises 500 mL of culture medium and 6000 IU/mL of IL-2, 30 μg of OKT-3, and between 5×10⁸ and 7.5×10⁸ antigen-presenting feeder cells per container.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 pg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments, a combination of IL-2, IL-7, IL-15, and/or IL-21 are employed as a combination during the second expansion. In some embodiments, IL-2, IL-7, IL-15, and/or IL-21 as well as any combinations thereof can be included during the second expansion, including, for example during a Step D processes according to FIG. 8 (in particular, e.g., FIG. 8B and/or FIG. 8C), as well as described herein. In some embodiments, a combination of IL-2, IL-15, and IL-21 are employed as a combination during the second expansion. In some embodiments, IL-2, IL-15, and IL-21 as well as any combinations thereof can be included during Step D processes according to FIG. 8 (in particular, e.g., FIG. 8B and/or FIG. 8C) and as described herein.

In some embodiments, the second expansion can be conducted in a supplemented cell culture medium comprising IL-2, OKT-3, antigen-presenting feeder cells, and optionally a TNFRSF agonist. In some embodiments, the second expansion occurs in a supplemented cell culture medium. In some embodiments, the supplemented cell culture medium comprises IL-2, OKT-3, and antigen-presenting feeder cells. In some embodiments, the second cell culture medium comprises IL-2, OKT-3, and antigen-presenting cells (APCs; also referred to as antigen-presenting feeder cells). In some embodiments, the second expansion occurs in a cell culture medium comprising IL-2, OKT-3, and antigen-presenting feeder cells (i.e., antigen presenting cells).

In some embodiments, the second expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the second expansion culture media comprises about 500 IU/mL of IL-15 to about 100 IU/mL of IL-15. In some embodiments, the second expansion culture media comprises about 400 IU/mL of IL-15 to about 100 IU/mL of IL-15. In some embodiments, the second expansion culture media comprises about 300 IU/mL of IL-15 to about 100 IU/mL of IL-15. In some embodiments, the second expansion culture media comprises about 200 IU/mL of IL-15. In some embodiments, the cell culture medium comprises about 180 IU/mL of IL-15. In an embodiment, the cell culture medium further comprises IL-15. In a preferred embodiment, the cell culture medium comprises about 180 IU/mL of IL-15.

In some embodiments, the second expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the second expansion culture media comprises about 20 IU/mL of IL-21 to about 0.5 IU/mL of IL-21. In some embodiments, the second expansion culture media comprises about 15 IU/mL of IL-21 to about 0.5 IU/mL of IL-21. In some embodiments, the second expansion culture media comprises about 12 IU/mL of IL-21 to about 0.5 IU/mL of IL-21. In some embodiments, the second expansion culture media comprises about 10 IU/mL of IL-21 to about 0.5 IU/mL of IL-21. In some embodiments, the second expansion culture media comprises about 5 IU/mL of IL-21 to about 1 IU/mL of IL-21. In some embodiments, the second expansion culture media comprises about 2 IU/mL of IL-21. In some embodiments, the cell culture medium comprises about 1 IU/mL of IL-21. In some embodiments, the cell culture medium comprises about 0.5 IU/mL of IL-21. In an embodiment, the cell culture medium further comprises IL-21. In a preferred embodiment, the cell culture medium comprises about 1 IU/mL of IL-21.

In some embodiments, the antigen-presenting feeder cells (APCs) are PBMCs. In an embodiment, the ratio of TILs to PBMCs and/or antigen-presenting cells in the rapid expansion and/or the second expansion is about 1 to 10, about 1 to 15, about 1 to 20, about 1 to 25, about 1 to 30, about 1 to 35, about 1 to 40, about 1 to 45, about 1 to 50, about 1 to 75, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to PBMCs in the rapid expansion and/or the second expansion is between 1 to 50 and 1 to 300. In an embodiment, the ratio of TILs to PBMCs in the rapid expansion and/or the second expansion is between 1 to 100 and 1 to 200.

In an embodiment, REP and/or the rapid second expansion is performed in flasks with the bulk TILs being mixed with a 100- or 200-fold excess of inactivated feeder cells, wherein the feeder cell concentration is at least 1.1 times (1.1×), 1.2×, 1.3×, 1.4×, 1.5×, 1.6×, 1.7×, 1.8×, 1.8×, 2×, 2.1×2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9× or 4.0× the feeder cell concentration in the priming first expansion, 30 ng/mL OKT3 anti-CD3 antibody and 6000 IU/mL IL-2 in 150 mL media. Media replacement is done (generally ⅔ media replacement via aspiration of ⅔ of spent media and replacement with an equal volume of fresh media) until the cells are transferred to an alternative growth chamber. Alternative growth chambers include G-REX flasks and gas permeable containers as more fully discussed below.

In some embodiments, the rapid second expansion (which can include processes referred to as the REP process) is 7 to 9 days, as discussed in the examples and figures. In some embodiments, the second expansion is 7 days. In some embodiments, the second expansion is 8 days. In some embodiments, the second expansion is 9 days.

In an embodiment, the second expansion (which can include expansions referred to as REP, as well as those referred to in Step D of FIG. 8 (in particular, e.g., FIG. 8B and/or FIG. 8C) may be performed in 500 mL capacity gas permeable flasks with 100 cm gas-permeable silicon bottoms (G-Rex 100, commercially available from Wilson Wolf Manufacturing Corporation, New Brighton, MN, USA), 5×10⁶ or 10×10⁶ TIL may be cultured with PBMCs in 400 mL of 50/50 medium, supplemented with 5% human AB serum, 3000 IU per mL of IL-2 and 30 ng per mL of anti-CD3 (OKT3). The G-Rex 100 flasks may be incubated at 37° C. in 5% CO₂. On day 5, 250 mL of supernatant may be removed and placed into centrifuge bottles and centrifuged at 1500 rpm (491×g) for 10 minutes. The TIL pellets may be re-suspended with 150 mL of fresh medium with 5% human AB serum, 6000 IU per mL of IL-2, and added back to the original GREX-100 flasks. When TILs are expanded serially in GREX-100 flasks, on day 10 or 11 the TILs can be moved to a larger flask, such as a GREX-500. The cells may be harvested on day 14 of culture. The cells may be harvested on day 15 of culture. The cells may be harvested on day 16 of culture. In some embodiments, media replacement is done until the cells are transferred to an alternative growth chamber. In some embodiments, 2/3 of the media is replaced by aspiration of spent media and replacement with an equal volume of fresh media. In some embodiments, alternative growth chambers include GREX flasks and gas permeable containers as more fully discussed below.

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium, CTS™ OpTmizer™ T-Cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CST™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%. 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM.

In some embodiments, the defined media described in International Patent Application Publication No. WO 1998/030679 and U.S. Patent Application Publication No. US 2002/0076747 A1, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the basal cell media is selected from the group consisting of Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1× Medium” in Table 4. In other embodiments, the non-trace element moiety ingredients in the defined medium are present in the final concentrations listed in the column under the heading “A Preferred Embodiment of the 1× Medium” in Table 4. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 4.

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 μM).

In some embodiments, the defined media described in Smith, et al., Clin Transl Immunology, 2015, 4(1), e31, the disclosures of which is incorporated by reference herein, are useful in the present invention. Briefly, RPMI or CTS™ OpTmizer™ was used as the basal cell medium, and supplemented with either 0, 2%, 5%, or 10% CTS™ Immune Cell Serum Replacement.

In an embodiment, the cell medium in the first and/or second gas permeable container is unfiltered. The use of unfiltered cell medium may simplify the procedures necessary to expand the number of cells. In an embodiment, the cell medium in the first and/or second gas permeable container lacks beta-mercaptoethanol (BME or PME; also known as 2-mercaptoethanol, CAS 60-24-2).

In an embodiment, the rapid second expansion (including expansions referred to as REP) is performed and further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

Optionally, a cell viability assay can be performed after the rapid second expansion (including expansions referred to as the REP expansion), using standard assays known in the art. For example, a trypan blue exclusion assay can be done on a sample of the bulk TILs, which selectively labels dead cells and allows a viability assessment. In some embodiments, TIL samples can be counted and viability determined using a Cellometer K2 automated cell counter (Nexcelom Bioscience, Lawrence, MA). In some embodiments, viability is determined according to the standard Cellometer K2 Image Cytometer Automatic Cell Counter protocol.

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained in the second expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β).

In some embodiments, the rapid second expansion culture medium (e.g., sometimes referred to as CM2 or the second cell culture medium), comprises IL-2, OKT-3, as well as the antigen-presenting feeder cells (APCs), as discussed in more detail below. In some embodiments, the rapid second expansion culture medium (e.g., sometimes referred to as CM2 or the second cell culture medium), comprises 6000 IU/mL IL-2, 30 ug/flask OKT-3, as well as 7.5×10⁸ antigen-presenting feeder cells (APCs), as discussed in more detail below. In some embodiments, the rapid second expansion culture medium (e.g., sometimes referred to as CM2 or the second cell culture medium), comprises IL-2, OKT-3, as well as the antigen-presenting feeder cells (APCs), as discussed in more detail below. In some embodiments, the rapid second expansion culture medium (e.g., sometimes referred to as CM2 or the second cell culture medium), comprises 6000 IU/mL IL-2, 30 ug/flask OKT-3, as well as 5×10⁸ antigen-presenting feeder cells (APCs), as discussed in more detail below.

In some embodiments, the rapid second expansion, for example, Step D according to FIG. 8 (in particular, e.g., FIG. 8B and/or FIG. 8C), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-100 or a G-REX-500. In some embodiments, the bioreactor employed is a G-REX-100. In some embodiments, the bioreactor employed is a G-REX-500.

### 1. Feeder Cells and Antigen Presenting Cells

In an embodiment, the rapid second expansion procedures described herein (for example including expansion such as those described in Step D from FIG. 8 (in particular, e.g., FIG. 8B and/or FIG. 8C), as well as those referred to as REP) require an excess of feeder cells during REP TIL expansion and/or during the rapid second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogeneic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and acceptable for use in the TIL expansion procedures described herein if the total number of viable cells on day 7 or 14 is less than the initial viable cell number put into culture on day 0 of the REP and/or day 0 of the second expansion (i.e., the start day of the second expansion).

In some embodiments, PBMCs are considered replication incompetent and acceptable for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/mL OKT3 antibody and 3000 IU/mL IL-2. In some embodiments, the PBMCs are cultured in the presence of 60 ng/mL OKT3 antibody and 6000 IU/mL IL-2. In some embodiments, the PBMCs are cultured in the presence of 60 ng/mL OKT3 antibody and 3000 IU/mL IL-2. In some embodiments, the PBMCs are cultured in the presence of 30 ng/mL OKT3 antibody and 6000 IU/mL IL-2.

In some embodiments, PBMCs are considered replication incompetent and acceptable for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30-60 ng/mL OKT3 antibody and 1000-6000 IU/mL IL-2. In some embodiments, the PBMCs are cultured in the presence of 30-60 ng/mL OKT3 antibody and 2000-5000 IU/mL IL-2. In some embodiments, the PBMCs are cultured in the presence of 30-60 ng/mL OKT3 antibody and 2000-4000 IU/mL IL-2. In some embodiments, the PBMCs are cultured in the presence of 30-60 ng/mL OKT3 antibody and 2500-3500 IU/mL IL-2. In some embodiments, the PBMCs are cultured in the presence of 30-60 ng/mL OKT3 antibody and 6000 IU/mL IL-2.

In some embodiments, the antigen-presenting feeder cells are PBMCs. In some embodiments, the antigen-presenting feeder cells are artificial antigen-presenting feeder cells. In an embodiment, the ratio of TILs to antigen-presenting feeder cells in the second expansion is about 1 to 10, about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In an embodiment, the ratio of TILs to antigen-presenting feeder cells in the second expansion is between 1 to 50 and 1 to 300. In an embodiment, the ratio of TILs to antigen-presenting feeder cells in the second expansion is between 1 to 100 and 1 to 200.

In an embodiment, the second expansion procedures described herein require a ratio of about 5×10⁸ feeder cells to about 100×10⁶ TILs. In an embodiment, the second expansion procedures described herein require a ratio of about 7.5×10⁸ feeder cells to about 100×10⁶ TILs. In another embodiment, the second expansion procedures described herein require a ratio of about 5×10⁸ feeder cells to about 50×10⁶ TILs. In another embodiment, the second expansion procedures described herein require a ratio of about 7.5×10⁸ feeder cells to about 50×10⁶ TILs. In yet another embodiment, the second expansion procedures described herein require about 5×10⁸ feeder cells to about 25×10⁶ TILs. In yet another embodiment, the second expansion procedures described herein require about 7.5×10⁸ feeder cells to about 25×10⁶ TILs. In yet another embodiment, the rapid second expansion requires twice the number of feeder cells as the rapid second expansion. In yet another embodiment, when the priming first expansion described herein requires about 2.5×10⁸ feeder cells, the rapid second expansion requires about 5×10⁸ feeder cells. In yet another embodiment, when the priming first expansion described herein requires about 2.5×10⁸ feeder cells, the rapid second expansion requires about 7.5×10⁸ feeder cells. In yet another embodiment, the rapid second expansion requires two times (2.0×), 2.5×, 3.0×, 3.5× or 4.0× the number of feeder cells as the priming first expansion.

In an embodiment, the rapid second expansion procedures described herein require an excess of feeder cells during the rapid second expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In an embodiment, artificial antigen-presenting (aAPC) cells are used in place of PBMCs. In some embodiments, the PBMCs are added to the rapid second expansion at twice the concentration of PBMCs that were added to the priming first expansion.

In general, the allogeneic PBMCs are inactivated, either via irradiation or heat treatment, and used in the TIL expansion procedures described herein, including the exemplary procedures described in the figures and examples.

In an embodiment, artificial antigen presenting cells are used in the rapid second expansion as a replacement for, or in combination with, PBMCs.

### 2. Cytokines and Other Additives

The rapid second expansion methods described herein generally use culture media with high doses of a cytokine, in particular IL-2, as is known in the art.

Alternatively, using combinations of cytokines for the rapid second expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21, and IL-2, IL-15 and IL-21, with the latter finding particular use in many embodiments. The use of combinations of cytokines specifically favors the generation of lymphocytes, and in particular T-cells as described therein.

In an embodiment, Step D may also include the addition of OKT-3 antibody or muromonab to the culture media, as described elsewhere herein. In an embodiment, Step D may also include the addition of a 4-1BB agonist to the culture media, as described elsewhere herein. In an embodiment, Step D may also include the addition of an OX-40 agonist to the culture media, as described elsewhere herein. In addition, additives such as peroxisome proliferator-activated receptor gamma coactivator I-alpha agonists, including proliferator-activated receptor (PPAR)-gamma agonists such as a thiazolidinedione compound, may be used in the culture media during Step D, as described in U.S. Patent Application Publication No. US 2019/0307796 A1, the disclosure of which is incorporated by reference herein.

### E. STEP E: Harvest TILs

After the rapid second expansion step, cells can be harvested. In some embodiments the TILs are harvested after one, two, three, four or more expansion steps, for example as provided in FIG. 8 (in particular, e.g., FIG. 8B and/or FIG. 8C). In some embodiments the TILs are harvested after two expansion steps, for example as provided in FIG. 8 (in particular, e.g., FIG. 8B and/or FIG. 8C). In some embodiments the TILs are harvested after two expansion steps, one priming first expansion and one rapid second expansion, for example as provided in FIG. 8 (in particular, e.g., FIG. 8B and/or FIG. 8C).

TILs can be harvested in any appropriate and sterile manner, including, for example by centrifugation. Methods for TIL harvesting are well known in the art and any such known methods can be employed with the present process. In some embodiments, TILs are harvested using an automated system.

Cell harvesters and/or cell processing systems are commercially available from a variety of sources, including, for example, Fresenius Kabi, Tomtec Life Science, Perkin Elmer, and Inotech Biosystems International, Inc. Any cell-based harvester can be employed with the present methods. In some embodiments, the cell harvester and/or cell processing system is a membrane-based cell harvester. In some embodiments, cell harvesting is via a cell processing system, such as the LOVO system (manufactured by Fresenius Kabi). The term “LOVO cell processing system” also refers to any instrument or device manufactured by any vendor that can pump a solution comprising cells through a membrane or filter such as a spinning membrane or spinning filter in a sterile and/or closed system environment, allowing for continuous flow and cell processing to remove supernatant or cell culture media without pelletization. In some embodiments, the cell harvester and/or cell processing system can perform cell separation, washing, fluid-exchange, concentration, and/or other cell processing steps in a closed, sterile system.

In some embodiments, the rapid second expansion, for example, Step D according to FIG. 8 (in particular, e.g., FIG. 8B and/or FIG. 8C), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-100 or a G-REX-500. In some embodiments, the bioreactor employed is a G-REX-100. In some embodiments, the bioreactor employed is a G-REX-500.

In some embodiments, Step E according to FIG. 8 (in particular, e.g., FIG. 8B and/or FIG. 8C), is performed according to the processes described herein. In some embodiments, the closed system is accessed via syringes under sterile conditions in order to maintain the sterility and closed nature of the system. In some embodiments, a closed system as described herein is employed.

In some embodiments, TILs are harvested according to the methods described in herein. In some embodiments, TILs between days 14 and 16 are harvested using the methods as described herein. In some embodiments, TILs are harvested at 14 days using the methods as described herein. In some embodiments, TILs are harvested at 15 days using the methods as described herein. In some embodiments, TILs are harvested at 16 days using the methods as described herein.

### F. STEP F: Final Formulation and Transfer to Infusion Container

After Steps A through E as provided in an exemplary order in FIG. 8 (in particular, e.g., FIG. 8B) and as outlined in detailed above and herein are complete, cells are transferred to a container for use in administration to a patient, such as an infusion bag or sterile vial. In some embodiments, once a therapeutically sufficient number of TILs are obtained using the expansion methods described above, they are transferred to a container for use in administration to a patient.

In an embodiment, TILs expanded using the methods of the present disclosure are administered to a patient as a pharmaceutical composition. In an embodiment, the pharmaceutical composition is a suspension of TILs in a sterile buffer. TILs expanded as disclosed herein may be administered by any suitable route as known in the art. In some embodiments, the TILs are administered as a single intra-arterial or intravenous infusion, which preferably lasts approximately 30 to 60 minutes. Other suitable routes of administration include intraperitoneal, intrathecal, and intralymphatic administration.

### V. Further Gen 2, Gen 3, and Other TIL Manufacturing Process Embodiments

This section describes alternative embodiments of the Gen 2, Gen 3, and other TIL manufacturing processes that may be used with the CCRs, chemokine receptors, and other embodiments of the present invention.

### A. PBMC Feeder Cell Ratios

In some embodiments, the culture media used in expansion methods described herein (see for example, FIG. 8 (in particular, e.g., FIG. 8B and/or FIG. 8C)) include an anti-CD3 antibody e.g. OKT-3. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J. Immunol. 1985, 135, 1719, hereby incorporated by reference in its entirety.

In an embodiment, the number of PBMC feeder layers is calculated as follows:
A. Volume of a T-cell (10 μm diameter): V=(4/3) πr³=523.6 μm³
B. Column of G-Rex 100 (M) with a 40 μm (4 cells) height: V=(4/3) πr³=4×10¹² μm³
C. Number cells required to fill column B: 4×10¹² μm³/523.6 μm³=7.6×10⁸ μm³*0.64=4.86×10⁸
D. Number cells that can be optimally activated in 4D space: 4.86×10⁸/24=20.25×10⁶
E. Number of feeders and TIL extrapolated to G-Rex 500: TIL: 100×10⁶ and Feeder: 2.5×10⁹

In this calculation, an approximation of the number of mononuclear cells required to provide an icosahedral geometry for activation of TIL in a cylinder with a 100 cm² base is used. The calculation derives the experimental result of ˜5×10⁸ for threshold activation of T-cells which closely mirrors NCI experimental data, as described in Jin, et. al., J. Immunother. 2012, 35, 283-292. In (C), the multiplier (0.64) is the random packing density for equivalent spheres as calculated by Jaeger and Nagel, Science, 1992, 255, 1523-3. In (D), the divisor 24 is the number of equivalent spheres that could contact a similar object in 4-dimensional space or “the Newton number” as described in Musin, Russ. Math. Surv., 2003, 58, 794-795.

In an embodiment, the number of antigen-presenting feeder cells exogenously supplied during the priming first expansion is approximately one-half the number of antigen-presenting feeder cells exogenously supplied during the rapid second expansion. In certain embodiments, the method comprises performing the priming first expansion in a cell culture medium which comprises approximately 50% fewer antigen presenting cells as compared to the cell culture medium of the rapid second expansion.

In another embodiment, the number of antigen-presenting feeder cells (APCs) exogenously supplied during the rapid second expansion is greater than the number of APCs exogenously supplied during the priming first expansion.

In another embodiment, the ratio of the number of APCs exogenously supplied during the rapid second expansion to the number of APCs exogenously supplied during the priming first expansion is selected from a range of from at or about 1.1:1 to at or about 20:1.

In another embodiment, the ratio of the number of APCs exogenously supplied during the rapid second expansion to the number of APCs exogenously supplied during the priming first expansion is selected from a range of from at or about 1.1:1 to at or about 10:1.

In another embodiment, the ratio of the number of APCs exogenously supplied during the rapid second expansion to the number of APCs exogenously supplied during the priming first expansion is selected from a range of from at or about 1.1:1 to at or about 9:1.

In another embodiment, the ratio of the number of APCs exogenously supplied during the rapid second expansion to the number of APCs exogenously supplied during the priming first expansion is selected from a range of from at or about 1.1:1 to at or about 8:1.

In another embodiment, the ratio of the number of APCs exogenously supplied during the rapid second expansion to the number of APCs exogenously supplied during the priming first expansion is selected from a range of from at or about 1.1:1 to at or about 7:1.

In another embodiment, the ratio of the number of APCs exogenously supplied during the rapid second expansion to the number of APCs exogenously supplied during the priming first expansion is selected from a range of from at or about 1.1:1 to at or about 6:1.

In another embodiment, the ratio of the number of APCs exogenously supplied during the rapid second expansion to the number of APCs exogenously supplied during the priming first expansion is selected from a range of from at or about 1.1:1 to at or about 5:1.

In another embodiment, the ratio of the number of APCs exogenously supplied during the rapid second expansion to the number of APCs exogenously supplied during the priming first expansion is selected from a range of from at or about 1.1:1 to at or about 4:1.

In another embodiment, the ratio of the number of APCs exogenously supplied during the rapid second expansion to the number of APCs exogenously supplied during the priming first expansion) is selected from a range of from at or about 1.1:1 to at or about 3:1.

In another embodiment, the ratio of the number of APCs exogenously supplied during the rapid second expansion to the number of APCs exogenously supplied during the priming first expansion is selected from a range of from at or about 1.1:1 to at or about 2.9:1.

In another embodiment, the ratio of the number of APCs exogenously supplied during the rapid second expansion to the number of APCs exogenously supplied during the priming first expansion is selected from a range of from at or about 1.1:1 to at or about 2.8:1.

In another embodiment, the ratio of the number of APCs exogenously supplied during the rapid second expansion to the number of APCs exogenously supplied during the priming first expansion is selected from a range of from at or about 1.1:1 to at or about 2.7:1.

In another embodiment, the ratio of the number of APCs exogenously supplied during the rapid second expansion to the number of APCs exogenously supplied during the priming first expansion is selected from a range of from at or about 1.1:1 to at or about 2.6:1.

In another embodiment, the ratio of the number of APCs exogenously supplied during the rapid second expansion to the number of APCs exogenously supplied during the priming first expansion is selected from a range of from at or about 1.1:1 to at or about 2.5:1.

In another embodiment, the ratio of the number of APCs exogenously supplied during the rapid second expansion to the number of APCs exogenously supplied during the priming first expansion is selected from a range of from at or about 1.1:1 to at or about 2.4:1.

In another embodiment, the ratio of the number of APCs exogenously supplied during the rapid second expansion to the number of APCs exogenously supplied during the priming first expansion is selected from a range of from at or about 1.1:1 to at or about 2.3:1.

In another embodiment, the ratio of the number of APCs exogenously supplied during the rapid second expansion to the number of APCs exogenously supplied during the priming first expansion is selected from a range of from at or about 1.1:1 to at or about 2.2:1.

In another embodiment, the ratio of the number of APCs exogenously supplied during the rapid second expansion to the number of APCs exogenously supplied during the priming first expansion is selected from a range of from at or about 1.1:1 to at or about 2.1:1.

In another embodiment, the ratio of the number of APCs exogenously supplied during the rapid second expansion to the number of APCs exogenously supplied during the priming first expansion is selected from a range of from at or about 1.1:1 to at or about 2:1.

In another embodiment, the ratio of the number of APCs exogenously supplied during the rapid second expansion to the number of APCs exogenously supplied during the priming first expansion is selected from a range of from at or about 2:1 to at or about 10:1.

In another embodiment, the ratio of the number of APCs exogenously supplied during the rapid second expansion to the number of APCs exogenously supplied during the priming first expansion is selected from a range of from at or about 2:1 to at or about 5:1.

In another embodiment, the ratio of the number of APCs exogenously supplied during the rapid second expansion to the number of APCs exogenously supplied during the priming first expansion is selected from a range of from at or about 2:1 to at or about 4:1.

In another embodiment, the ratio of the number of APCs exogenously supplied during the rapid second expansion to the number of APCs exogenously supplied during the priming first expansion is selected from a range of from at or about 2:1 to at or about 3:1.

In another embodiment, the ratio of the number of APCs exogenously supplied during the rapid second expansion to the number of APCs exogenously supplied during the priming first expansion is selected from a range of from at or about 2:1 to at or about 2.9:1.

In another embodiment, the ratio of the number of APCs exogenously supplied during the rapid second expansion to the number of APCs exogenously supplied during the priming first expansion is selected from a range of from at or about 2:1 to at or about 2.8:1.

In another embodiment, the ratio of the number of APCs exogenously supplied during the rapid second expansion to the number of APCs exogenously supplied during the priming first expansion is selected from a range of from at or about 2:1 to at or about 2.7:1.

In another embodiment, the ratio of the number of APCs exogenously supplied during the rapid second expansion to the number of APCs exogenously supplied during the priming first expansion is selected from a range of from at or about 2:1 to at or about 2.6:1.

In another embodiment, the ratio of the number of APCs exogenously supplied during the rapid second expansion to the number of APCs exogenously supplied during the priming first expansion is selected from a range of from at or about 2:1 to at or about 2.5:1.

In another embodiment, the ratio of the number of APCs exogenously supplied during the rapid second expansion to the number of APCs exogenously supplied during the priming first expansion is selected from a range of from at or about 2:1 to at or about 2.4:1.

In another embodiment, the ratio of the number of APCs exogenously supplied during the rapid second expansion to the number of APCs exogenously supplied during the priming first expansion is selected from a range of from at or about 2:1 to at or about 2.3:1.

In another embodiment, the ratio of the number of APCs exogenously supplied during the rapid second expansion to the number of APCs exogenously supplied during the priming first expansion is selected from a range of from at or about 2:1 to at or about 2.2:1.

In another embodiment, the ratio of the number of APCs exogenously supplied during the rapid second expansion to the number of APCs exogenously supplied during the priming first expansion is selected from a range of from at or about 2:1 to at or about 2.1:1.

In another embodiment, the ratio of the number of APCs exogenously supplied during the rapid second expansion to the number of APCs exogenously supplied during the priming first expansion is at or about 2:1.

In another embodiment, the ratio of the number of APCs exogenously supplied during the rapid second expansion to the number of APCs exogenously supplied during the priming first expansion is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In another embodiment, the number of APCs exogenously supplied during the priming first expansion is at or about 1×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸ or 3.5×10⁸ APCs, and the number of APCs exogenously supplied during the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs.

In another embodiment, the number of APCs exogenously supplied during the priming first expansion is selected from the range of at or about 1.5×10⁸ APCs to at or about 3×10⁸ APCs, and the number of APCs exogenously supplied during the rapid second expansion is selected from the range of at or about 4×10⁸ APCs to at or about 7.5×10⁸ APCs.

In another embodiment, the number of APCs exogenously supplied during the priming first expansion is selected from the range of at or about 2×10⁸ APCs to at or about 2.5×10⁸ APCs, and the number of APCs exogenously supplied during the rapid second expansion is selected from the range of at or about 4.5×10⁸ APCs to at or about 5.5×10⁸ APCs.

In another embodiment, the number of APCs exogenously supplied during the priming first expansion is at or about 2.5×10⁸ APCs, and the number of APCs exogenously supplied during the rapid second expansion is at or about 5×10⁸ APCs.

In an embodiment, the number of APCs (including, for example, PBMCs) added at day 0 of the priming first expansion is approximately one-half of the number of PBMCs added at day 7 of the priming first expansion (e.g., day 7 of the method). In certain embodiments, the method comprises adding antigen presenting cells at day 0 of the priming first expansion to the first population of TILs and adding antigen presenting cells at day 7 to the second population of TILs, wherein the number of antigen presenting cells added at day 0 is approximately 50% of the number of antigen presenting cells added at day 7 of the priming first expansion (e.g., day 7 of the method).

In another embodiment, the number of APCs (including, for example, PBMCs) exogenously supplied at day 7 of the rapid second expansion is greater than the number of PBMCs exogenously supplied at day 0 of the priming first expansion.

In another embodiment, the APCs exogenously supplied in the priming first expansion are seeded in the culture flask at a density selected from a range of at or about 1.0×10⁶ APCs/cm² to at or about 4.5×10⁶ APCs/cm².

In another embodiment, the APCs exogenously supplied in the priming first expansion are seeded in the culture flask at a density selected from a range of at or about 1.5×10⁶ APCs/cm² to at or about 3.5×10⁶ APCs/cm².

In another embodiment, the APCs exogenously supplied in the priming first expansion are seeded in the culture flask at a density selected from a range of at or about 2×10⁶ APCs/cm² to at or about 3×10⁶ APCs/cm².

In another embodiment, the APCs exogenously supplied in the priming first expansion are seeded in the culture flask at a density of at or about 2×10⁶ APCs/cm².

In another embodiment, the APCs exogenously supplied in the priming first expansion are seeded in the culture flask at a density of at or about 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶, 3.1×10⁶, 3.2/106 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶ 4.3×10⁶, 4.4×10⁶ or 4.5×10⁶ APCs/cm².

In another embodiment, the APCs exogenously supplied in the rapid second expansion are seeded in the culture flask at a density selected from a range of at or about 2.5×10⁶ APCs/cm² to at or about 7.5×10⁶ APCs/cm².

In another embodiment, the APCs exogenously supplied in the rapid second expansion are seeded in the culture flask at a density selected from a range of at or about 3.5×10⁶ APCs/cm² to about 6.0×10⁶ APCs/cm².

In another embodiment, the APCs exogenously supplied in the rapid second expansion are seeded in the culture flask at a density selected from a range of at or about 4.0×10⁶ APCs/cm² to about 5.5×10⁶ APCs/cm².

In another embodiment, the APCs exogenously supplied in the rapid second expansion are seeded in the culture flask at a density selected from a range of at or about 4.0×10⁶ APCs/cm².

In another embodiment, the APCs exogenously supplied in the rapid second expansion are seeded in the culture flask at a density of at or about 2.5×10⁶ APCs/cm², 2.6×10⁶ APCs/cm², 2.7×10⁶ APCs/cm², 2.8×10⁶, 2.9×10⁶, 3×10⁶, 3.1×10⁶, 3.2×10⁶, 3.3×10⁶, 3.4×10⁶, 3.5×10⁶ 3.6×10⁶, 3.7×10⁶ 3.8×10⁶, 3.9×10⁶ 4×10⁶ 4.1×10⁶, 4.2×10⁶, 4.3×10⁶ 4.4×10⁶, 4.5×10⁶, 4.6×10⁶, 4.7×10⁶, 4.8×10⁶, 4.9×10⁶, 5×10⁶, 5.1×10⁶, 5.2×10⁶, 5.3×10⁶, 5.4×10⁶, 5.5×10⁶ 5.6×10⁶ 5.7×10⁶ 5.8×10⁶, 5.9×10⁶ 6×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7×10⁶, 7.1×10⁶, 7.2×10⁶, 7.3×10⁶, 7.4×10⁶ or 7.5×10⁶ APCs/cm².

In another embodiment, the APCs exogenously supplied in the priming first expansion are seeded in the culture flask at a density of at or about 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶ 3.1×10⁶, 3.2×10⁶, 3.3×10⁶, 3.4×10⁶ 3.5×10⁶, 3.6×10⁶, 3.7×10⁶ 3.8×10⁶, 3.9×10⁶, 4×10⁶ 4.1×10⁶, 4.2×10⁶ 4.3×10⁶, 4.4×10⁶ or 4.5×10⁶ APCs/cm² and the APCs exogenously supplied in the rapid second expansion are seeded in the culture flask at a density of at or about 2.5×10⁶ APCs/cm², 2.6×10⁶ APCs/cm², 2.7×10⁶ APCs/cm², 2.8×10⁶, 2.9×10⁶, 3×10⁶, 3.1×10⁶ 3.2×10⁶, 3.3×10⁶ 3.4×10⁶, 3.5×10⁶, 3.6×10⁶, 3.7×10⁶, 3.8×10⁶, 3.9×10⁶ 4×10⁶, 4.1×10⁶ 4.2×10⁶, 4.3×10⁶ 4.4×10⁶, 4.5×10⁶, 4.6×10⁶, 4.7×10⁶, 4.8×10⁶, 4.9×10⁶, 5×10⁶, 5.1×10⁶, 5.2×10⁶, 5.3×10⁶ 5.4×10⁶ 5.5×10⁶ 5.6×10⁶, 5.7×10⁶ 5.8×10⁶ 5.9×10⁶ 6×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7×10⁶, 7.1×10⁶, 7.2×10⁶, 7.3×10⁶, 7.4×10⁶ or 7.5×10⁶ APCs/cm².

In another embodiment, the APCs exogenously supplied in the priming first expansion are seeded in the culture flask at a density selected from a range of at or about 1.0×10⁶ APCs/cm² to at or about 4.5×10⁶ APCs/cm², and the APCs exogenously supplied in the rapid second expansion are seeded in the culture flask at a density selected from a range of at or about 2.5×10⁶ APCs/cm² to at or about 7.5×10⁶ APCs/cm².

In another embodiment, the APCs exogenously supplied in the priming first expansion are seeded in the culture flask at a density selected from a range of at or about 1.5×10⁶ APCs/cm² to at or about 3.5×10⁶ APCs/cm², and the APCs exogenously supplied in the rapid second expansion are seeded in the culture flask at a density selected from a range of at or about 3.5×10⁶ APCs/cm² to at or about 6×10⁶ APCs/cm².

In another embodiment, the APCs exogenously supplied in the priming first expansion are seeded in the culture flask at a density selected from a range of at or about 2×10⁶ APCs/cm² to at or about 3×10⁶ APCs/cm², and the APCs exogenously supplied in the rapid second expansion are seeded in the culture flask at a density selected from a range of at or about 4×10⁶ APCs/cm² to at or about 5.5×10⁶ APCs/cm².

In another embodiment, the APCs exogenously supplied in the priming first expansion are seeded in the culture flask at a density at or about 2×10⁶ APCs/cm² and the APCs exogenously supplied in the rapid second expansion are seeded in the culture flask at a density of at or about 4×10⁶ APCs/cm².

In another embodiment, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 20:1.

In another embodiment, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 10:1.

In another embodiment, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 9:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 8:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 7:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 6:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 5:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 4:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 3:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.9:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.8:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.7:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.6:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.5:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.4:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.3:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.2:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.1:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 10:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 5:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 4:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 3:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.9:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.8:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.7:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.6:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.5:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.4:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.3:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.2:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.1:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is at or about 2:1.

In another embodiment, the ratio of the number of APCs (including, for example, PBMCs) exogenously supplied at day 7 of the rapid second expansion to the number of APCs (including, for example, PBMCs) exogenously supplied at day 0 of the priming first expansion is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In another embodiment, the number of APCs (including, for example, PBMCs) exogenously supplied at day 0 of the priming first expansion is at or about 1×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸ or 3.5×10⁸ APCs (including, for example, PBMCs), and the number of APCs (including, for example, PBMCs) exogenously supplied at day 7 of the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs (including, for example, PBMCs).

In another embodiment, the number of APCs (including, for example, PBMCs) exogenously supplied at day 0 of the priming first expansion is selected from the range of at or about 1×10⁸ APCs (including, for example, PBMCs) to at or about 3.5×10⁸ APCs (including, for example, PBMCs), and the number of APCs (including, for example, PBMCs) exogenously supplied at day 7 of the rapid second expansion is selected from the range of at or about 3.5×10⁸ APCs (including, for example, PBMCs) to at or about 1×10⁹ APCs (including, for example, PBMCs).

In another embodiment, the number of APCs (including, for example, PBMCs) exogenously supplied at day 0 of the priming first expansion is selected from the range of at or about 1.5×10⁸ APCs to at or about 3×10⁸ APCs (including, for example, PBMCs), and the number of APCs (including, for example, PBMCs) exogenously supplied at day 7 of the rapid second expansion is selected from the range of at or about 4×10⁸ APCs (including, for example, PBMCs) to at or about 7.5×10⁸ APCs (including, for example, PBMCs).

In another embodiment, the number of APCs (including, for example, PBMCs) exogenously supplied at day 0 of the priming first expansion is selected from the range of at or about 2×10⁸ APCs (including, for example, PBMCs) to at or about 2.5×10⁸ APCs (including, for example, PBMCs), and the number of APCs (including, for example, PBMCs) exogenously supplied at day 7 of the rapid second expansion is selected from the range of at or about 4.5×10⁸ APCs (including, for example, PBMCs) to at or about 5.5×10⁸ APCs (including, for example, PBMCs).

In another embodiment, the number of APCs (including, for example, PBMCs) exogenously supplied at day 0 of the priming first expansion is at or about 2.5×10⁸ APCs (including, for example, PBMCs) and the number of APCs (including, for example, PBMCs) exogenously supplied at day 7 of the rapid second expansion is at or about 5×10⁸ APCs (including, for example, PBMCs).

In an embodiment, the number of layers of APCs (including, for example, PBMCs) added at day 0 of the priming first expansion is approximately one-half of the number of layers of APCs (including, for example, PBMCs) added at day 7 of the rapid second expansion. In certain embodiments, the method comprises adding antigen presenting cell layers at day 0 of the priming first expansion to the first population of TILs and adding antigen presenting cell layers at day 7 to the second population of TILs, wherein the number of antigen presenting cell layer added at day 0 is approximately 50% of the number of antigen presenting cell layers added at day 7.

In another embodiment, the number of layers of APCs (including, for example, PBMCs) exogenously supplied at day 7 of the rapid second expansion is greater than the number of layers of APCs (including, for example, PBMCs) exogenously supplied at day 0 of the priming first expansion.

In another embodiment, day 0 of the priming first expansion occurs in the presence of layered APCs (including, for example, PBMCs) with an average thickness of at or about 2 cell layers and day 7 of the rapid second expansion occurs in the presence of layered APCs (including, for example, PBMCs) with an average thickness of at or about 4 cell layers.

In another embodiment, day 0 of the priming first expansion occurs in the presence of layered APCs (including, for example, PBMCs) with an average thickness of at or about one cell layer and day 7 of the rapid second expansion occurs in the presence of layered APCs (including, for example, PBMCs) with an average thickness of at or about 3 cell layers.

In another embodiment, day 0 of the priming first expansion occurs in the presence of layered APCs (including, for example, PBMCs) with an average thickness of at or about 1.5 cell layers to at or about 2.5 cell layers and day 7 of the rapid second expansion occurs in the presence of layered APCs (including, for example, PBMCs) with an average thickness of at or about 3 cell layers.

In another embodiment, day 0 of the priming first expansion occurs in the presence of layered APCs (including, for example, PBMCs) with an average thickness of at or about one cell layer and day 7 of the rapid second expansion occurs in the presence of layered APCs (including, for example, PBMCs) with an average thickness of at or about 2 cell layers.

In another embodiment, day 0 of the priming first expansion occurs in the presence of layered APCs (including, for example, PBMCs) with an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers and day 7 of the rapid second expansion occurs in the presence of layered APCs (including, for example, PBMCs) with an average thickness of at or about 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In another embodiment, day 0 of the priming first expansion occurs in the presence of layered APCs (including, for example, PBMCs) with an average thickness of at or about 1 cell layer to at or about 2 cell layers and day 7 of the rapid second expansion occurs in the presence of layered APCs (including, for example, PBMCs) with an average thickness of at or about 3 cell layers to at or about 10 cell layers.

In another embodiment, day 0 of the priming first expansion occurs in the presence of layered APCs (including, for example, PBMCs) with an average thickness of at or about 2 cell layers to at or about 3 cell layers and day 7 of the rapid second expansion occurs in the presence of layered APCs (including, for example, PBMCs) with an average thickness of at or about 4 cell layers to at or about 8 cell layers.

In another embodiment, day 0 of the priming first expansion occurs in the presence of layered APCs (including, for example, PBMCs) with an average thickness of at or about 2 cell layers and day 7 of the rapid second expansion occurs in the presence of layered APCs (including, for example, PBMCs) with an average thickness of at or about 4 cell layers to at or about 8 cell layers.

In another embodiment, day 0 of the priming first expansion occurs in the presence of layered APCs (including, for example, PBMCs) with an average thickness of at or about 1, 2 or 3 cell layers and day 7 of the rapid second expansion occurs in the presence of layered APCs (including, for example, PBMCs) with an average thickness of at or about 3, 4, 5, 6, 7, 8, 9 or 10 cell layers.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:10.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:8.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:7.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:6.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:5.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:4.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:3.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:2.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.2 to at or about 1:8.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.3 to at or about 1:7.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.4 to at or about 1:6.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.5 to at or about 1:5.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.6 to at or about 1:4.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.7 to at or about 1:3.5.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.8 to at or about 1:3.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.9 to at or about 1:2.5.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is at or about 1:2.

In another embodiment, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from at or about 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9 or 1:10.

In some embodiments, the number of APCs in the priming first expansion is selected from the range of about 1.0×10⁶ APCs/cm² to about 4.5×10⁶ APCs/cm², and the number of APCs in the rapid second expansion is selected from the range of about 2.5×10⁶ APCs/cm² to about 7.5×10⁶ APCs/cm².

In some embodiments, the number of APCs in the priming first expansion is selected from the range of about 1.5×10⁶ APCs/cm² to about 3.5×10⁶ APCs/cm², and the number of APCs in the rapid second expansion is selected from the range of about 3.5×10⁶ APCs/cm² to about 6.0×10⁶ APCs/cm².

In some embodiments, the number of APCs in the priming first expansion is selected from the range of about 2.0×10⁶ APCs/cm² to about 3.0×10⁶ APCs/cm², and the number of APCs in the rapid second expansion is selected from the range of about 4.0×10⁶ APCs/cm² to about 5.5×10⁶ APCs/cm².

### B. Optional Cell Medium Components

### 1. Anti-CD3 Antibodies

In some embodiments, the culture media used in expansion methods described herein (including those referred to as REP, see for example, FIGS. 1 and 8 (in particular, e.g., FIG. 8B)) include an anti-CD3 antibody. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J. Immunol. 1985, 135, 1719, hereby incorporated by reference in its entirety.

As will be appreciated by those in the art, there are a number of suitable anti-human CD3 antibodies that find use in the invention, including anti-human CD3 polyclonal and monoclonal antibodies from various mammals, including, but not limited to, murine, human, primate, rat, and canine antibodies. In some embodiments, the OKT3 anti-CD3 antibody muromonab is used (commercially available from Ortho-McNeil, Raritan, NJ or Miltenyi Biotech, Auburn, CA). In some embodiments, the anti-CD3 antibody, such as OKT-3, is added during the pre-REP stage or initial REP stage (of a Gen 3 method) immediately after tumor fragments or digest is added to the media of the first gas permeable flask, bag, or other container.

As will be appreciated by those in the art, there are a number of suitable anti-human CD3 antibodies that find use in the invention, including anti-human CD3 polyclonal and monoclonal antibodies from various mammals, including, but not limited to, murine, human, primate, rat, and canine antibodies. In some embodiments, the OKT3 anti-CD3 antibody muromonab is used (commercially available from Ortho-McNeil, Raritan, NJ or Miltenyi Biotech, Auburn, CA).

### 2. 4-1BB (CD137) Agonists

In an embodiment, the cell culture medium of the first expansion and/or the rapid second expansion comprises a TNFRSF agonist. In an embodiment, the TNFRSF agonist is a 4-1BB (CD137) agonist. The 4-1BB agonist may be any 4-1BB binding molecule known in the art. The 4-1BB binding molecule may be a monoclonal antibody or fusion protein capable of binding to human or mammalian 4-1BB. The 4-1BB agonists or 4-1BB binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The 4-1BB agonist or 4-1BB binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to 4-1BB. In an embodiment, the 4-1BB agonist is an antigen binding protein that is a fully human antibody. In an embodiment, the 4-1BB agonist is an antigen binding protein that is a humanized antibody. In some embodiments, 4-1BB agonists for use in the presently disclosed methods and compositions include anti-4-1BB antibodies, human anti-4-1BB antibodies, mouse anti-4-1BB antibodies, mammalian anti-4-1BB antibodies, monoclonal anti-4-1BB antibodies, polyclonal anti-4-1BB antibodies, chimeric anti-4-1BB antibodies, anti-4-1BB adnectins, anti-4-1BB domain antibodies, single chain anti-4-1BB fragments, heavy chain anti-4-1BB fragments, light chain anti-4-1BB fragments, anti-4-1BB fusion proteins, and fragments, derivatives, conjugates, variants, or biosimilars thereof. Agonistic anti-4-1BB antibodies are known to induce strong immune responses. Lee, et al., PLOS One 2013, 8, e69677. In a preferred embodiment, the 4-1BB agonist is an agonistic, anti-4-1BB humanized or fully human monoclonal antibody (i.e., an antibody derived from a single cell line). In an embodiment, the 4-1BB agonist is EU-101 (Eutilex Co. Ltd.), utomilumab, or urelumab, or a fragment, derivative, conjugate, variant, or biosimilar thereof. In a preferred embodiment, the 4-1BB agonist is utomilumab or urelumab, or a fragment, derivative, conjugate, variant, or biosimilar thereof.

In a preferred embodiment, the 4-1BB agonist or 4-1BB binding molecule may also be a fusion protein. In a preferred embodiment, a multimeric 4-1BB agonist, such as a trimeric or hexameric 4-1BB agonist (with three or six ligand binding domains), may induce superior receptor (4-1BBL) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic 4-1B13 antibodies and fusion proteins are known to induce strong immune responses. In a preferred embodiment, the 4-1BB agonist is a monoclonal antibody or fusion protein that binds specifically to 4-1BB antigen in a manner sufficient to reduce toxicity. In some embodiments, the 4-1BB agonist is an agonistic 4-1BB monoclonal antibody or fusion protein that abrogates antibody-dependent cellular toxicity (ADCC), for example NK cell cytotoxicity. In some embodiments, the 4-1BB agonist is an agonistic 4-1BB monoclonal antibody or fusion protein that abrogates antibody-dependent cell phagocytosis (ADCP). In some embodiments, the 4-1BB agonist is an agonistic 4-1BB monoclonal antibody or fusion protein that abrogates complement-dependent cytotoxicity (CDC). In some embodiments, the 4-1BB agonist is an agonistic 4-1BB monoclonal antibody or fusion protein which abrogates Fc region functionality.

In some embodiments, the 4-1BB agonists are characterized by binding to human 4-18BB (SEQ ID NO:40) with high affinity and agonistic activity. In an embodiment, the 4-1BB agonist is a binding molecule that binds to human 4-1BB (SEQ ID NO:40). In an embodiment, the 4-1BB agonist is a binding molecule that binds to murine 4-1BB (SEQ ID NO:41). The amino acid sequences of 4-1BB antigen to which a 4-1BB agonist or binding molecule binds are summarized in Table 5.

TABLE 5
Amino acid sequences of 4-1BB antigens.

| Identifier | Sequence (One-Letter Amino Acid Symbols) | |
|---|---|---|
| SEQ ID NO: 40 | MGNSCYNIVA TLLLVLNFER TRSLQDPCSN CPAGTFCDNN RNQICSPCPP NSFSSAGGQR | 60 |
| human 4-1BB, | TCDICRQCKG VFRTRKECSS TSNAECDCTP GFHCLGAGCS MCEQDCKQGQ ELTKKGCKDC | 120 |
| Tumor necrosis | CFGTFNDQKR GICRPWTNCS LDGKSVLVNG TKERDVVCGP SPADLSPGAS SVTPPAPARE | 180 |
| factor receptor | PGHSPQIISF FLALTSTALL FLLFFITLRF SVVKRGRKKL LYIFKQPFMR PVQTTQEEDG | 240 |
| superfamily, | CSCRFPEEEE GGCEL | 255 |
| member 9 (Homo | | |
| sapiens) | | |
| SEQ ID NO: 41 | MGNNCYNVVV IVLLLVGCEK VGAVQNSCDN CQPGTFCRKY NPVCKSCPPS TFSSIGGQPN | 60 |
| murine 4-1BB, | CNICRVCAGY FRFKKFCSST HNAECECIEG FHCLGPQCTR CEKDCRPGQE LTKQGCKTCS | 120 |
| Tumor necrosis | LGTFNDQNGT GVCRPWTNCS LDGRSVLKTG TTEKDVVCGP PVVSFSPSTT ISVTPEGGPG | 180 |
| factor receptor | GHSLQVLTLF LALTSALLLA LIFITILFSV LKWIRKKFPH IFKQPFKKTT GAAQEEDACS | 240 |
| superfamily, | CRCPQEEEGG GGGYEL | 256 |
| member 9 (Mus | | |
| musculus) | | |

In some embodiments, the compositions, processes and methods described include a 4-1BB agonist that binds human or murine 4-1BB with a K_{D} of about 100 pM or lower, binds human or murine 4-1BB with a K_{D} of about 90 pM or lower, binds human or murine 4-1BB with a K_{D} of about 80 pM or lower, binds human or murine 4-1BB with a K_{D} of about 70 pM or lower, binds human or murine 4-1BB with a K_{D} of about 60 pM or lower, binds human or murine 4-1BB with a K_{D} of about 50 pM or lower, binds human or murine 4-1BB with a K_{D} of about 40 pM or lower, or binds human or murine 4-1BB with a K_{D} of about 30 pM or lower.

In some embodiments, the compositions, processes and methods described include a 4-1BB agonist that binds to human or murine 4-1BB with a k_{assoc} of about 7.5×10⁵ l/M·s or faster, binds to human or murine 4-1BB with a k_{assoc} of about 7.5×10⁵ l/M·s or faster, binds to human or murine 4-1BB with a k_{assoc} of about 8×10⁵ l/M·s or faster, binds to human or murine 4-1BB with a k_{assoc} of about 8.5×10⁵ l/M·s or faster, binds to human or murine 4-1BB with a k_{assoc} of about 9×10⁵ l/M·s or faster, binds to human or murine 4-1BB with a k_{assoc} of about 9.5×10⁵ l/M·s or faster, or binds to human or murine 4-1BB with a k_{assoc} of about 1×10⁶ l/M·s or faster.

In some embodiments, the compositions, processes and methods described include a 4-1BB agonist that binds to human or murine 4-1BB with a k_{dissoc} of about 2×10⁻⁵ l/s or slower, binds to human or murine 4-1BB with a k_{dissoc} of about 2.1×10⁻⁵ l/s or slower, binds to human or murine 4-1BB with a k_{dissoc} of about 2.2×10⁻⁵ l/s or slower, binds to human or murine 4-1BB with a k_{dissoc} of about 2.3×10⁻⁵ l/s or slower, binds to human or murine 4-1BB with a k_{dissoc} of about 2.4×10⁻⁵ l/s or slower, binds to human or murine 4-1BB with a k_{dissoc} of about 2.5×10⁻⁵ l/s or slower, binds to human or murine 4-1BB with a k_{dissoc} of about 2.6×10⁻⁵ l/s or slower or binds to human or murine 4-11B with a k_{dissoc} of about 2.7×10⁻⁵ l/s or slower, binds to human or murine 4-1BB with a k_{dissoc} of about 2.8×10⁻⁵ l/s or slower, binds to human or murine 4-1BB with a k_{dissoc} of about 2.9×10⁻⁵ l/s or slower, or binds to human or murine 4-1BB with a k_{dissoc} of about 3×10⁻⁵ l/s or slower.

In some embodiments, the compositions, processes and methods described include a 4-1BB agonist that binds to human or murine 4-1BB with an IC₅₀ of about 10 nM or lower, binds to human or murine 4-1BB with an IC₅₀ of about 9 nM or lower, binds to human or murine 4-1BB with an IC₅₀ of about 8 nM or lower, binds to human or murine 4-1BB with an IC₅₀ of about 7 nM or lower, binds to human or murine 4-1BB with an IC₅₀ of about 6 nM or lower, binds to human or murine 4-1BB with an IC₅₀ of about 5 nM or lower, binds to human or murine 4-1BB with an IC₅₀ of about 4 nM or lower, binds to human or murine 4-1BB with an IC₅₀ of about 3 nM or lower, binds to human or murine 4-1BB with an IC₅₀ of about 2 nM or lower, or binds to human or murine 4-1BB with an IC₅₀ of about 1 nM or lower.

In a preferred embodiment, the 4-1BB agonist is utomilumab, also known as PF-05082566 or MOR-7480, or a fragment, derivative, variant, or biosimilar thereof. Utomilumab is available from Pfizer, Inc. Utomilumab is an immunoglobulin G2-lambda, anti-[Homo sapiens TNFRSF9 (tumor necrosis factor receptor (TNFR) superfamily member 9, 4-1BB, T cell antigen ILA, CD137)], Homo sapiens (fully human) monoclonal antibody. The amino acid sequences of utomilumab are set forth in Table 6. Utomilumab comprises glycosylation sites at Asn59 and Asn292; heavy chain intrachain disulfide bridges at positions 22-96 (V_{H}-V_{L}), 143-199 (C_{H}1-C_{L}), 256-316 (C_{H}2) and 362-420 (C_{H}3); light chain intrachain disulfide bridges at positions 22′-87′ (V_{H}-V_{L}) and 136′-195′ (C_{H}1-C_{L}); interchain heavy chain-heavy chain disulfide bridges at IgG2A isoform positions 218-218, 219-219, 222-222, and 225-225, at IgG2A/B isoform positions 218-130, 219-219, 222-222, and 225-225, and at IgG23 isoform positions 219-130 (2), 222-222, and 225-225; and interchain heavy chain-light chain disulfide bridges at IgG2A isoform positions 130-213′ (2), IgG2A/B isoform positions 218-213′ and 130-213′, and at IgG2B isoform positions 218-213′ (2). The preparation and properties of utomilumab and its variants and fragments are described in U.S. Pat. Nos. 8,821,867; 8,337,850; and 9,468,678, and International Patent Application Publication No. WO 2012/032433 A1, the disclosures of each of which are incorporated by reference herein. Preclinical characteristics of utomilumab are described in Fisher, et al., Cancer Immunolog. & Immunother. 2012, 61, 1721-33. Current clinical trials of utomilumab in a variety of hematological and solid tumor indications include U.S. National Institutes of Health clinicaltrials.gov identifiers NCT02444793, NCT01307267, NCT02315066, and NCT02554812.

In an embodiment, a 4-1BB agonist comprises a heavy chain given by SEQ ID NO:42 and a light chain given by SEQ ID NO:43. In an embodiment, a 4-1BB agonist comprises heavy and light chains having the sequences shown in SEQ ID NO:42 and SEQ ID NO:43, respectively, or antigen binding fragments, Fab fragments, single-chain variable fragments (scFv), variants, or conjugates thereof. In an embodiment, a 4-1BB agonist comprises heavy and light chains that are each at least 99% identical to the sequences shown in SEQ ID NO:42 and SEQ ID NO:43, respectively. In an embodiment, a 4-1BB agonist comprises heavy and light chains that are each at least 98% identical to the sequences shown in SEQ ID NO:42 and SEQ ID NO:43, respectively. In an embodiment, a 4-1BB agonist comprises heavy and light chains that are each at least 97% identical to the sequences shown in SEQ ID NO:42 and SEQ ID NO:43, respectively. In an embodiment, a 4-1BB agonist comprises heavy and light chains that are each at least 96% identical to the sequences shown in SEQ ID NO:42 and SEQ ID NO:43, respectively. In an embodiment, a 4-1BB agonist comprises heavy and light chains that are each at least 95% identical to the sequences shown in SEQ ID NO:42 and SEQ ID NO:43, respectively.

In an embodiment, the 4-1BB agonist comprises the heavy and light chain CDRs or variable regions (VRs) of utomilumab. In an embodiment, the 4-1BB agonist heavy chain variable region (V_{H}) comprises the sequence shown in SEQ ID NO:44, and the 4-1BB agonist light chain variable region (V_{L}) comprises the sequence shown in SEQ ID NO:45, and conservative amino acid substitutions thereof. In an embodiment, a 4-1BB agonist comprises V_{H} and V_{L} regions that are each at least 99% identical to the sequences shown in SEQ ID NO:44 and SEQ ID NO:45, respectively. In an embodiment, a 4-1BB agonist comprises V_{H} and V_{L} regions that are each at least 98% identical to the sequences shown in SEQ ID NO:44 and SEQ ID NO:45, respectively. In an embodiment, a 4-1BB agonist comprises V_{H} and V_{L} regions that are each at least 97% identical to the sequences shown in SEQ ID NO:44 and SEQ ID NO:45, respectively. In an embodiment, a 4-1BB agonist comprises V_{H} and V_{L} regions that are each at least 96% identical to the sequences shown in SEQ ID NO:44 and SEQ ID NO:45, respectively. In an embodiment, a 4-1BB agonist comprises V_{H} and V_{L} regions that are each at least 95% identical to the sequences shown in SEQ ID NO:44 and SEQ ID NO:45, respectively. In an embodiment, a 4-1BB agonist comprises an scFv antibody comprising V_{H} and V_{L} regions that are each at least 99% identical to the sequences shown in SEQ ID NO:44 and SEQ ID NO:45.

In an embodiment, a 4-1BB agonist comprises heavy chain CDR1, CDR2 and CDR3 domains having the sequences set forth in SEQ ID NO:46, SEQ ID NO:47, and SEQ ID NO:48, respectively, and conservative amino acid substitutions thereof, and light chain CDR1, CDR2 and CDR3 domains having the sequences set forth in SEQ ID NO:49, SEQ ID NO:50, and SEQ ID NO:51, respectively, and conservative amino acid substitutions thereof.

In an embodiment, the 4-1BB agonist is a 4-1BB agonist biosimilar monoclonal antibody approved by drug regulatory authorities with reference to utomilumab. In an embodiment, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is utomilumab. In some embodiments, the one or more post-translational modifications are selected from one or more of: glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is utomilumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab.

TABLE 6
Amino acid sequences for 4-1BB agonist antibodies related to utomilumab.

| Identifier | Sequence (One-Letter Amino Acid Symbols) | |
|---|---|---|
| SEQ ID NO: 42 | EVQLVQSGAE VKKPGESLRI SCKGSGYSFS TYWISWVRQM PGKGLEWMGK IYPGDSYTNY | 60 |
| heavy chain for | SPSFQGQVTI SADKSISTAY LQWSSLKASD TAMYYCARGY GIFDYWGQGT LVTVSSASTK | 120 |
| utomilumab | GPSVFPLAPC SRSTSESTAA LGCLVKDYFP EPVTVSWNSG ALTSGVHTFP AVLQSSGLYS | 180 |
| | LSSVVTVPSS NFGTQTYTCN VDHKPSNTKV DKTVERKCCV ECPPCPAPPV AGPSVFLFPP | 240 |
| | KPKDTLMISR TPEVTCVVVD VSHEDPEVQF NWYVDGVEVH NAKTKPREEQ FNSTFRVVSV | 300 |
| | LTVVHQDWLN GKEYKCKVSN KGLPAPIEKT ISKTKGQPRE PQVYTLPPSR EEMTKNQVSL | 360 |
| | TCLVKGFYPS DIAVEWESNG QPENNYKTTP PMLDSDGSFF LYSKLTVDKS RWQQGNVFSC | 420 |
| | SVMHEALHNH YTQKSLSLSP G | 441 |
| SEQ ID NO: 43 | SYELTQPPSV SVSPGQTASI TCSGDNIGDQ YAHWYQQKPG QSPVLVIYQD KNRPSGIPER | 60 |
| light chain for | FSGSNSGNTA TLTISGTQAM DEADYYCATY TGFGSLAVFG GGTKLTVLGQ PKAAPSVTLF | 120 |
| utomilumab | PPSSEELQAN KATLVCLISD FYPGAVTVAW KADSSPVKAG VETTTPSKQS NNKYAASSYL | 180 |
| | SLTPEQWKSH RSYSCQVTHE GSTVEKTVAP TECS | 214 |
| SEQ ID NO: 44 | EVQLVQSGAE VKKPGESLRI SCKGSGYSFS TYWISWVRQM PGKGLEWMG KIYPGDSYTN | 60 |
| heavy chain | YSPSFQGQVT ISADKSISTA YLQWSSLKAS DTAMYYCARG YGIFDYWGQ GTLVTVSS | 108 |
| variable region | | |
| for utomilumab | | |
| SEQ ID NO: 45 | SYELTQPPSV SVSPGQTASI TCSGDNIGDQ YAHWYQQKPG QSPVLVIYQD KNRPSGIPER | 60 |
| light chain | FSGSNSGNTA TLTISGTQAM DEADYYCATY TGFGSLAVFG GGTKLTVL | 108 |
| variable region | | |
| for utomilumab | | |
| SEQ ID NO: 46 | STYWIS | 6 |
| heavy chain CDR1 | | |
| for utomilumab | | |
| SEQ ID NO: 47 | KIYPGDSYTN YSPSFQG | 17 |
| heavy chain CDR2 | | |
| for utomilumab | | |
| SEQ ID NO: 48 | RGYGIFDY | 8 |
| heavy chain CDR3 | | |
| for utomilumab | | |
| SEQ ID NO: 49 | SGDNIGDQYA H | 11 |
| light chain CDR1 | | |
| for utomilumab | | |
| SEQ ID NO: 50 | QDKNRPS | 7 |
| light chain CDR2 | | |
| for utomilumab | | |
| SEQ ID NO: 51 | ATYTGFGSLA V | 11 |
| light chain CDR3 | | |
| for utomilumab | | |

In a preferred embodiment, the 4-1BB agonist is the monoclonal antibody urelumab, also known as BMS-663513 and 20H4.9.h4a, or a fragment, derivative, variant, or biosimilar thereof. Urelumab is available from Bristol-Myers Squibb, Inc., and Creative Biolabs, Inc. Urelumab is an immunoglobulin G4-kappa, anti-[Homo sapiens TNFRSF9 (tumor necrosis factor receptor superfamily member 9, 4-1BB, T cell antigen ILA, CD137)], Homo sapiens (fully human) monoclonal antibody. The amino acid sequences of urelumab are set forth in Table 7. Urelumab comprises N-glycosylation sites at positions 298 (and 298″); heavy chain intrachain disulfide bridges at positions 22-95 (V_{H}-V_{L}), 148-204 (C_{H}1-C_{L}), 262-322 (C_{H}2) and 368-426 (C_{H}3) (and at positions 22″-95″, 148″-204″, 262″-322″, and 368″-426″); light chain intrachain disulfide bridges at positions 23′-88′ (V_{H}-V_{L}) and 136′-196′ (C_{H}1-C_{L}) (and at positions 23′″-88′″ and 136′″-196′″); interchain heavy chain-heavy chain disulfide bridges at positions 227-227″ and 230-230″; and interchain heavy chain-light chain disulfide bridges at 135-216′ and 135″-216′″. The preparation and properties of urelumab and its variants and fragments are described in U.S. Pat. Nos. 7,288,638 and 8,962,804, the disclosures of which are incorporated by reference herein. The preclinical and clinical characteristics of urelumab are described in Segal, et al., Clin. Cancer Res. 2016, available at http:/dx.doi.org/10.1158/1078-0432.CCR-16-1272. Current clinical trials of urelumab in a variety of hematological and solid tumor indications include U.S. National Institutes of Health clinicaltrials.gov identifiers NCT01775631, NCT02110082, NCT02253992, and NCT01471210.

In an embodiment, a 4-1BB agonist comprises a heavy chain given by SEQ ID NO:52 and a light chain given by SEQ ID NO:53. In an embodiment, a 4-1BB agonist comprises heavy and light chains having the sequences shown in SEQ ID NO:52 and SEQ ID NO:53, respectively, or antigen binding fragments, Fab fragments, single-chain variable fragments (scFv), variants, or conjugates thereof. In an embodiment, a 4-1BB agonist comprises heavy and light chains that are each at least 99% identical to the sequences shown in SEQ ID NO:52 and SEQ ID NO:53, respectively. In an embodiment, a 4-1BB agonist comprises heavy and light chains that are each at least 98% identical to the sequences shown in SEQ ID NO:52 and SEQ ID NO:53, respectively. In an embodiment, a 4-1BB agonist comprises heavy and light chains that are each at least 97% identical to the sequences shown in SEQ ID NO:52 and SEQ ID NO:53, respectively. In an embodiment, a 4-1BB agonist comprises heavy and light chains that are each at least 96% identical to the sequences shown in SEQ ID NO:52 and SEQ ID NO:53, respectively. In an embodiment, a 4-1BB agonist comprises heavy and light chains that are each at least 95% identical to the sequences shown in SEQ ID NO:52 and SEQ ID NO:53, respectively.

In an embodiment, the 4-1BB agonist comprises the heavy and light chain CDRs or variable regions (VRs) of urelumab. In an embodiment, the 4-1BB agonist heavy chain variable region (V_{H}) comprises the sequence shown in SEQ ID NO:54, and the 4-1BB agonist light chain variable region (V_{L}) comprises the sequence shown in SEQ ID NO:55, and conservative amino acid substitutions thereof. In an embodiment, a 4-1BB agonist comprises V_{H} and V_{L} regions that are each at least 99% identical to the sequences shown in SEQ ID NO:54 and SEQ ID NO:55, respectively. In an embodiment, a 4-1BB agonist comprises V_{H} and V_{L} regions that are each at least 98% identical to the sequences shown in SEQ ID NO:54 and SEQ ID NO:55, respectively. In an embodiment, a 4-1BB agonist comprises V_{H} and V_{L} regions that are each at least 97% identical to the sequences shown in SEQ ID NO:54 and SEQ ID NO:55, respectively. In an embodiment, a 4-1BB agonist comprises V_{H} and V_{L} regions that are each at least 96% identical to the sequences shown in SEQ ID NO:54 and SEQ ID NO:55, respectively. In an embodiment, a 4-1BB agonist comprises V_{H} and V_{L} regions that are each at least 95% identical to the sequences shown in SEQ ID NO:54 and SEQ ID NO:55, respectively. In an embodiment, a 4-1BB agonist comprises an scFv antibody comprising V_{H} and V_{L} regions that are each at least 99% identical to the sequences shown in SEQ ID NO:54 and SEQ ID NO:55.

In an embodiment, a 4-1BB agonist comprises heavy chain CDR1, CDR2 and CDR3 domains having the sequences set forth in SEQ ID NO:56, SEQ ID NO:57, and SEQ ID NO:58, respectively, and conservative amino acid substitutions thereof, and light chain CDR1, CDR2 and CDR3 domains having the sequences set forth in SEQ ID NO:59, SEQ ID NO:60, and SEQ ID NO:61, respectively, and conservative amino acid substitutions thereof.

In an embodiment, the 4-1BB agonist is a 4-1BB agonist biosimilar monoclonal antibody approved by drug regulatory authorities with reference to urelumab. In an embodiment, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is urelumab. In some embodiments, the one or more post-translational modifications are selected from one or more of: glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is urelumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab.

TABLE 7
Amino acid sequences for 4-1BB agonist antibodies related to urelumab.

| Identifier | Sequence (One-Letter Amino Acid Symbols) | |
|---|---|---|
| SEQ ID NO: 52 | QVQLQQWGAG LLKPSETLSL TCAVYGGSFS GYYWSWIRQS PEKGLEWIGE INHGGYVTYN | 60 |
| heavy chain for | PSLESRVTIS VDTSKNQFSL KLSSVTAADT AVYYCARDYG PGNYDWYFDL WGRGTLVTVS | 120 |
| urelumab | SASTKGPSVF PLAPCSRSTS ESTAALGCLV KDYFPEPVTV SWNSGALTSG VHTFPAVLQS | 180 |
| | SGLYSLSSVV TVPSSSLGTK TYTCNVDHKP SNTKVDKRVE SKYGPPCPPC PAPEFLGGPS | 240 |
| | VFLFPPKPKD TLMISRTPEV TCVVVDVSQE DPEVQFNWYV DGVEVHNAKT KPREEQFNST | 300 |
| | YRVVSVLTVL HQDWINGKEY KCKVSNKGLP SSIEKTISKA KGQPREPQVY TLPPSQEEMT | 360 |
| | KNQVSLTCLV KGFYPSDIAV EWESNGQPEN NYKTTPPVLD SDGSFFLYSR LTVDKSRWQE | 420 |
| | GNVFSCSVMH EALHNHYTQK SLSLSLGK | 448 |
| SEQ ID NO: 53 | EIVLTQSPAT LSLSPGERAT LSCRASQSVS SYLAWYQQKP GQAPRLLIYD ASNRATGIPA | 60 |
| light chain for | RFSGSGSGTD FTLTISSLEP EDFAVYYCQQ RSNWPPALTF CGGTKVEIKR TVAAPSVFIF | 120 |
| urelumab | PPSDEQLKSG TASVVCLLNN FYPREAKVQW KVDNALQSGN SQESVTEQDS KDSTYSLSST | 180 |
| | LTLSKADYEK HKVYACEVTH QGLSSPVTKS FNRGEC | 216 |
| SEQ ID NO: 54 | MKHLWFFLLL VAAPRWVLSQ VQLQQWGAGL LKPSETLSLT CAVYGGSFSG YYWSWIRQSP | 60 |
| variable heavy | EKGLEWIGEI NHGGYVTYNP SLESRVTISV DTSKNQFSLK LSSVTAADTA VYYCARDYGP | 120 |
| chain for | | |
| urelumab | | |
| SEQ ID NO: 55 | MEAPAQLLFL LLLWLPDTTG EIVLTQSPAT LSLSPGERAT LSCRASQSVS SYLAWYQQKP | 60 |
| variable light | QAPRLLIYD ASNRATGIPA RFSGSGSGTD FTLTISSLEP EDEAVYYCQQ | 110 |
| chain for | | |
| urelumab | | |
| SEQ ID NO: 56 | GYYWS | 5 |
| heavy chain CDR1 | | |
| for urelumab | | |
| SEQ ID NO: 57 | EINHGGYVTY NPSLES | 16 |
| heavy chain CDR2 | | |
| for urelumab | | |
| SEQ ID NO: 58 | DYGPGNYDWY FDL | 13 |
| heavy chain CDR3 | | |
| for urelumab | | |
| SEQ ID NO: 59 | RASQSVSSYL A | 11 |
| light chain CDR1 | | |
| for urelumab | | |
| SEQ ID NO: 60 | DASNRAT | 7 |
| light chain CDR2 | | |
| for urelumab | | |
| SEQ ID NO: 61 | QQRSDWPPAL T | 11 |
| light chain CDR3 | | |
| for urelumab | | |

In an embodiment, the 4-1BB agonist is selected from the group consisting of 1D8, 3Elor, 4B34 (BioLegend 309809), H4-1BB-M127 (BD Pharmingen 552532), BBK2 (Thermo Fisher MS621PABX), 145501 (Leinco Technologies B591), the antibody produced by cell line deposited as ATCC No. HB-11248 and disclosed in U.S. Pat. No. 6,974,863, 5F4 (BioLegend 31 1503), C65-485 (BD Pharmingen 559446), antibodies disclosed in U.S. Patent Application Publication No. US 2005/0095244, antibodies disclosed in U.S. Pat. No. 7,288,638 (such as 20H4.9-IgG1 (BMS-663031)), antibodies disclosed in U.S. Pat. No. 6,887,673 (such as 4E9 or BMS-554271), antibodies disclosed in U.S. Pat. No. 7,214,493, antibodies disclosed in U.S. Pat. No. 6,303,121, antibodies disclosed in U.S. Pat. No. 6,569,997, antibodies disclosed in U.S. Pat. No. 6,905,685 (such as 4E9 or BMS-554271), antibodies disclosed in U.S. Pat. No. 6,362,325 (such as 1D8 or BMS-469492; 3H3 or BMS-469497; or 3E1), antibodies disclosed in U.S. Pat. No. 6,974,863 (such as 53A2); antibodies disclosed in U.S. Pat. No. 6,210,669 (such as 1D8, 3B8, or 3E1), antibodies described in U.S. Pat. No. 5,928,893, antibodies disclosed in U.S. Pat. No. 6,303,121, antibodies disclosed in U.S. Pat. No. 6,569,997, antibodies disclosed in International Patent Application Publication Nos. WO 2012/177788, WO 2015/119923, and WO 2010/042433, and fragments, derivatives, conjugates, variants, or biosimilars thereof, wherein the disclosure of each of the foregoing patents or patent application publications is incorporated by reference here.

In an embodiment, the 4-1BB agonist is a 4-1BB agonistic fusion protein described in International Patent Application Publication Nos. WO 2008/025516 A1, WO 2009/007120 A1, WO 2010/003766 A1, WO 2010/010051 A1, and WO 2010/078966 A1; U.S. Patent Application Publication Nos. US 2011/0027218 A1, US 2015/0126709 A1, US 2011/0111494 A1, US 2015/0110734 A1, and US 2015/0126710 A1; and U.S. Pat. Nos. 9,359,420, 9,340,599, 8,921,519, and 8,450,460, the disclosures of which are incorporated by reference herein.

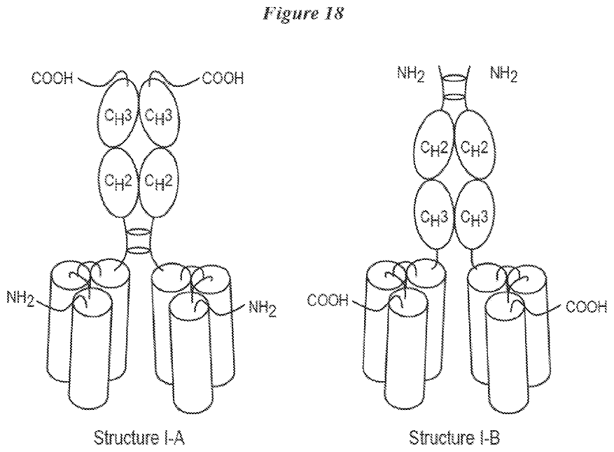

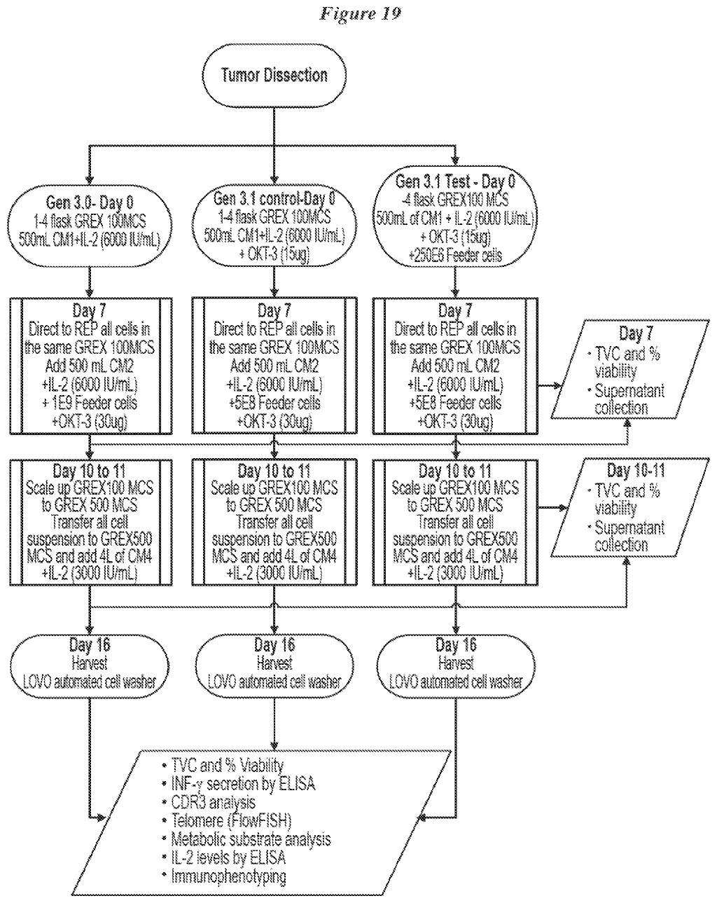

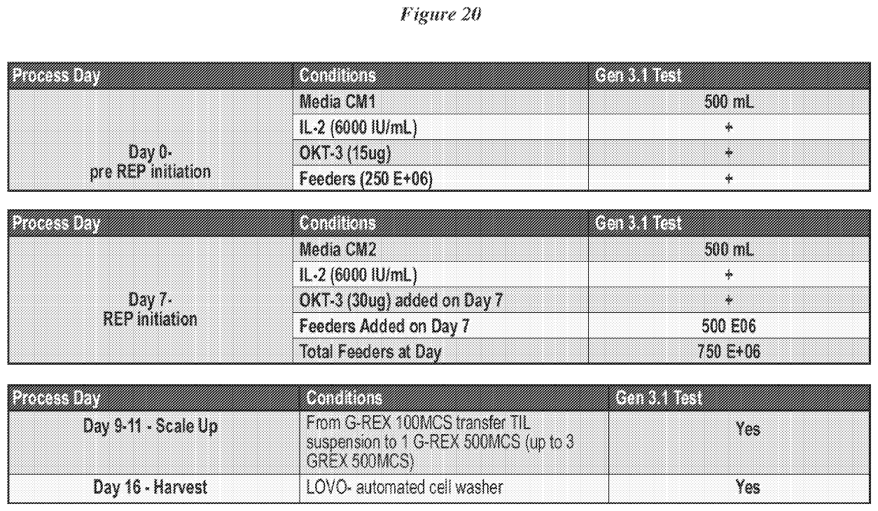

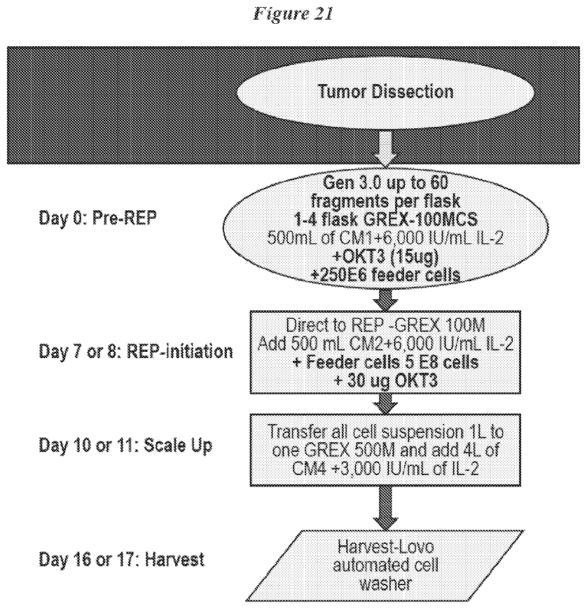

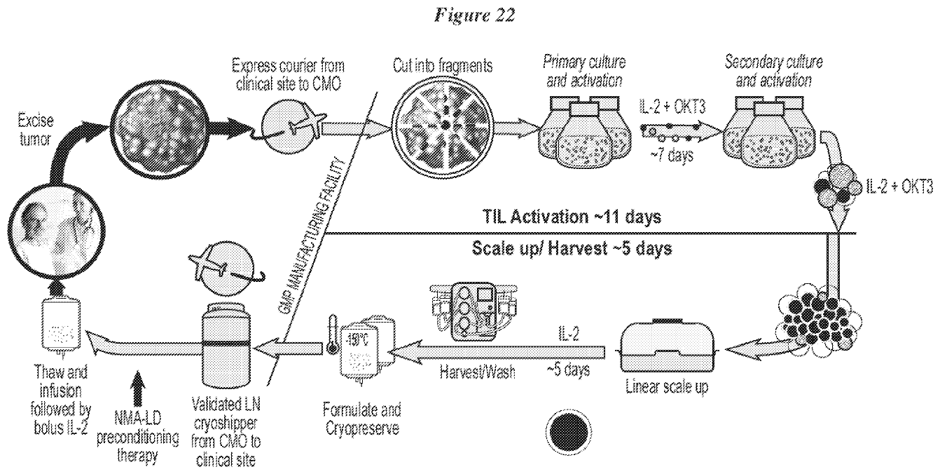

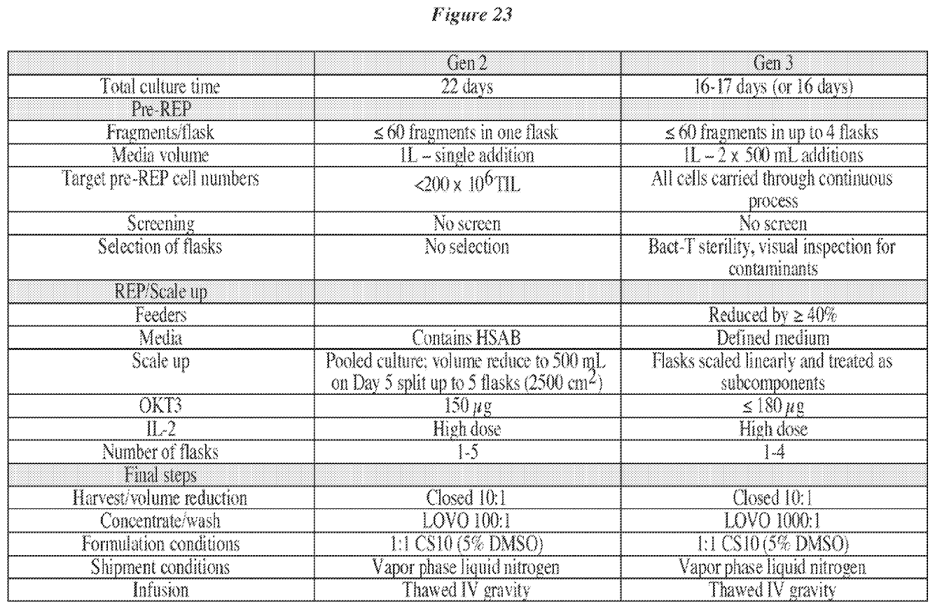

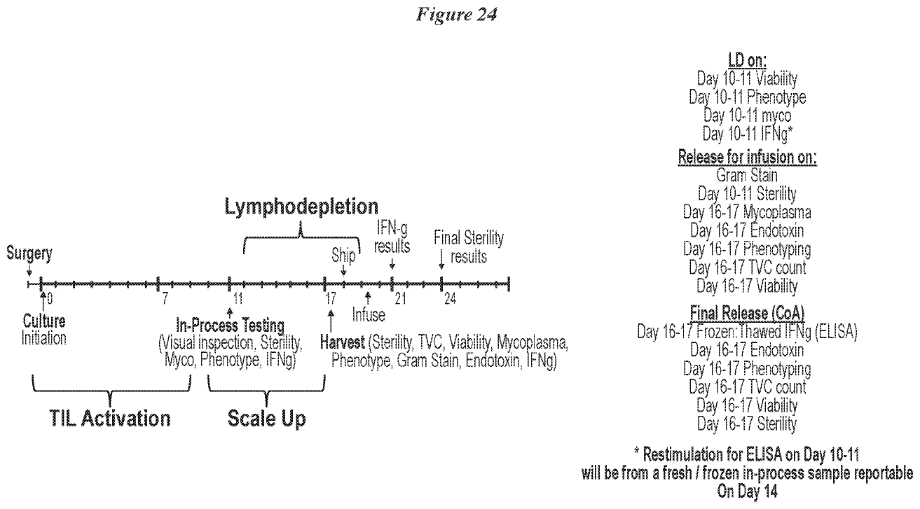

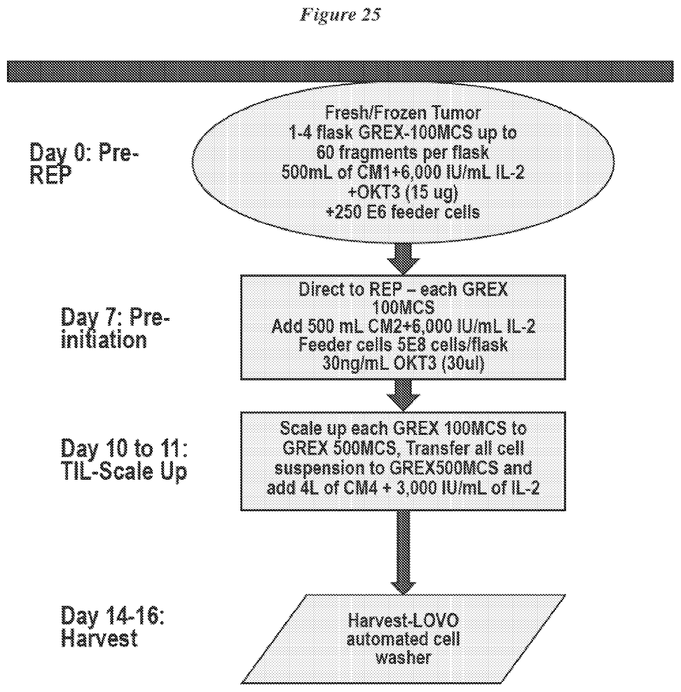

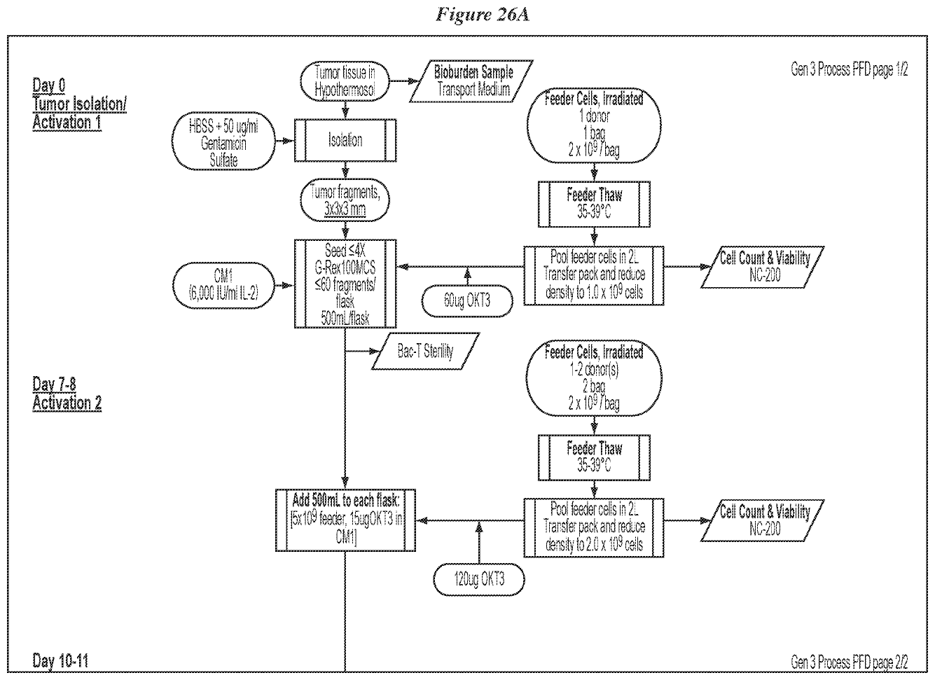

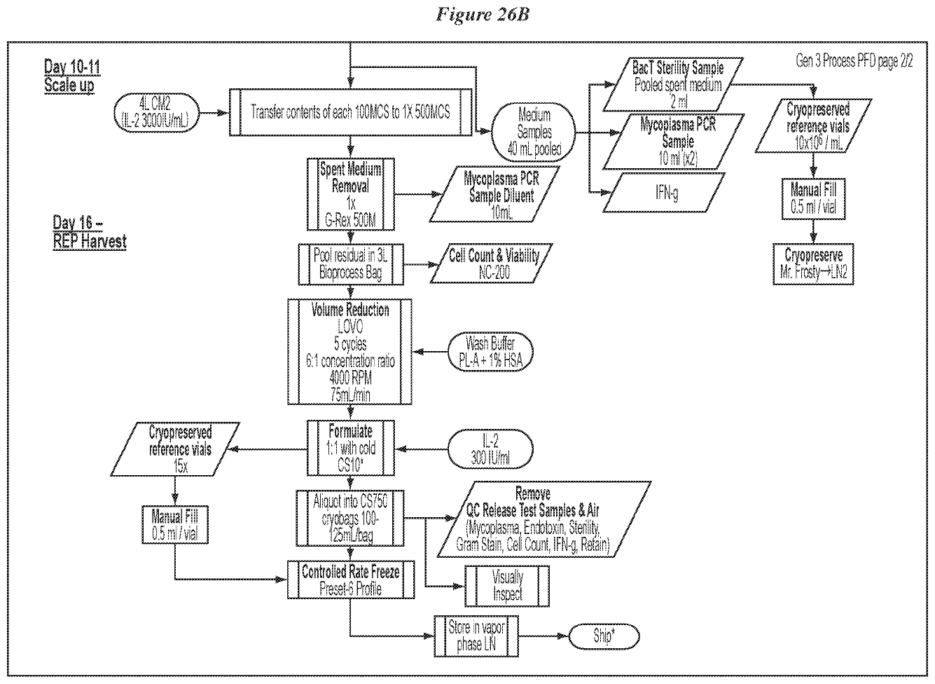

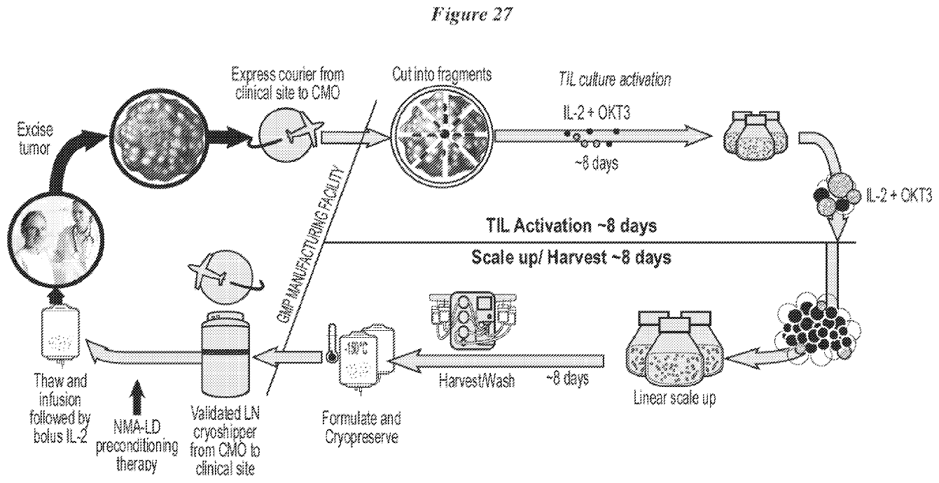

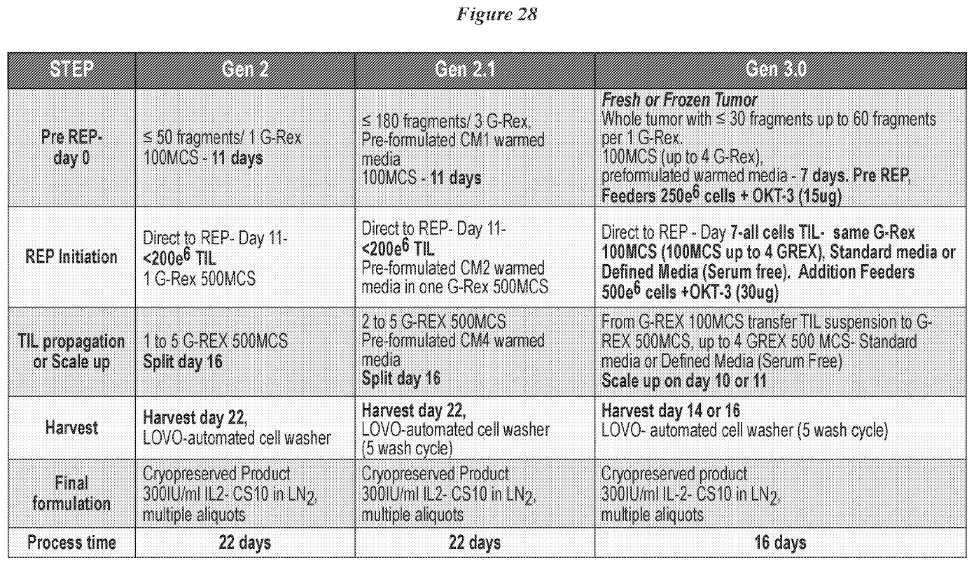

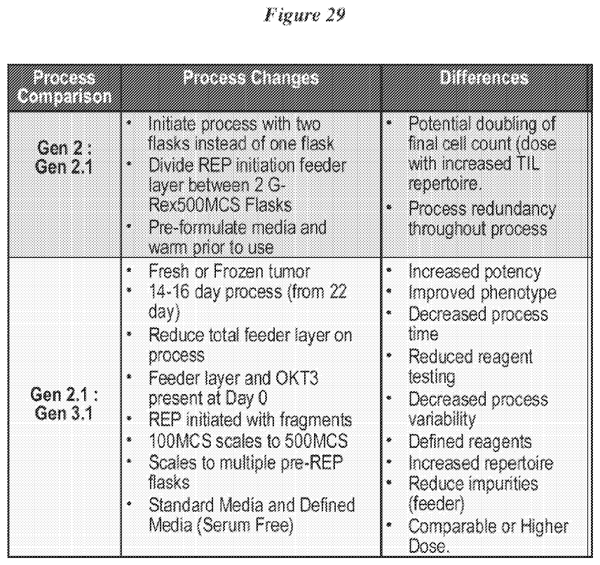

In an embodiment, the 4-1BB agonist is a 4-1BB agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof (see, FIG. 18). In structures I-A and I-B, the cylinders refer to individual polypeptide binding domains. Structures I-A and I-B comprise three linearly-linked TNFRSF binding domains derived from e.g., 4-1BBL (4-1BB ligand, CD137 ligand (CD137L), or tumor necrosis factor superfamily member 9 (TNFSF9)) or an antibody that binds 4-1BB, which fold to form a trivalent protein, which is then linked to a second triavelent protein through IgG1-Fc (including C_{H}3 and C_{H}2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a V_{H} and a V_{L} chain connected by a linker that may comprise hydrophilic residues and Gly and Ser sequences for flexibility, as well as Glu and Lys for solubility. Any scFv domain design may be used, such as those described in de Marco, Microbial Cell Factories, 2011, 10, 44; Ahmad, et al., Clin. & Dev. Immunol. 2012, 980250; Monnier, et al., Antibodies, 2013, 2, 193-208; or in references incorporated elsewhere herein. Fusion protein structures of this form are described in U.S. Pat. Nos. 9,359,420, 9,340,599, 8,921,519, and 8,450,460, the disclosures of which are incorporated by reference herein.

Amino acid sequences for the other polypeptide domains of structure I-A given in FIG. 18 are found in Table 8. The Fc domain preferably comprises a complete constant domain (amino acids 17-230 of SEQ ID NO:62) the complete hinge domain (amino acids 1-16 of SEQ ID NO:62) or a portion of the hinge domain (e.g., amino acids 4-16 of SEQ ID NO:62). Preferred linkers for connecting a C-terminal Fc-antibody may be selected from the embodiments given in SEQ ID NO:63 to SEQ ID NO:72, including linkers suitable for fusion of additional polypeptides.

TABLE 8
Amino acid sequences for TNFRSF agonist fusion proteins, including 4-1BB
agonist fusion proteins, with C-terminal Fc-antibody fragment fusion protein design
(structure I-A).

| Identifier | Sequence (One-Letter Amino Acid Symbols) | |
|---|---|---|
| SEQ ID NO: 62 | KSCDKTHTCP PCPAPELLGG PSVFLFPPKP KDTLMISRTP EVTCVVVDVS HEDPEVKFNW | 60 |
| Fc domain | YVDGVEVHNA KTKPREEQYN STYRVVSVLT VLHQDWLNGK EYKCKVSNKA LPAPIEKTIS | 120 |
| | KAKGQPREPQ VYTLPPSREE MTKNQVSLTC LVKGFYPSDI AVEWESNGQP ENNYKTTPPV | 180 |
| | LDSDGSFFLY SKLTVDKSRW QQGNVFSCSV MHEALHNHYT QKSLSLSPGK | 230 |
| SEQ ID NO: 63 | GGPGSSKSCD KTHTCPPCPA PE | 22 |
| linker | | |
| SEQ ID NO: 64 | GGSGSSKSCD KTHTCPPCPA PE | 22 |
| linker | | |
| SEQ ID NO: 65 | GGPGSSSSSS SKSCDKTHTC PPCPAPE | 27 |
| linker | | |
| SEQ ID NO: 66 | GGSGSSSSSS SKSCDKTHTC PPCPAPE | 27 |
| linker | | |
| SEQ ID NO: 67 | GGPGSSSSSS SSSKSCDKTH TCPPCPAPE | 29 |
| linker | | |
| SEQ ID NO: 68 | GGSGSSSSSS SSSKSCDKTH TCPPCPAPE | 29 |
| linker | | |
| SEQ ID NO: 69 | GGPGSSGSGS SDKTHTCPPC PAPE | 24 |
| linker | | |
| SEQ ID NO: 70 | GGPGSSGSGS DKTHTCPPCP APE | 23 |
| linker | | |
| SEQ ID NO: 71 | GGPSSSGSDK THTCPPCPAP E | 21 |
| linker | | |
| SEQ ID NO: 72 | GGSSSSSSSS GSDKTHTCPP CPAPE | 25 |
| linker | | |

Amino acid sequences for the other polypeptide domains of structure I-B given in FIG. 18 are found in Table 9. If an Fc antibody fragment is fused to the N-terminus of an TNRFSF fusion protein as in structure I-B, the sequence of the Fc module is preferably that shown in SEQ ID NO:73, and the linker sequences are preferably selected from those embodiments set forth in SED ID NO:74 to SEQ ID NO:76.

TABLE 9
Amino acid sequences for TNFRSF agonist fusion proteins, including 4-1BB
agonist fusion proteins, with N-terminal Fc-antibody fragment fusion protein design
(structure I-B).

| Identifier | Sequence (One-Letter Amino Acid Symbols) | |
|---|---|---|
| SEQ ID NO: 73 | METDTLLLWV LLLWVPAGNG DKTHTCPPCP APELLGGPSV FLFPPKPKDT LMISRTPEVT | 60 |
| Fc domain | CVVVDVSHED PEVKFNWYVD GVEVHNAKTK PREEQYNSTY RVVSVLTVLH QDWINGKEYK | 120 |
| | CKVSNKALPA PIEKTISKAK GQPREPQVYT LPPSREEMTK NQVSLTCLVK GFYPSDIAVE | 180 |
| | WESNGQPENN YKTTPPVLDS DGSFFLYSKL TVDKSRWQQG NVFSCSVMHE ALHNHYTQKS | 240 |
| | LSLSPG | 246 |
| SEQ ID NO: 74 | SGSGSGSGSG S | 11 |
| linker | | |
| SEQ ID NO: 75 | SSSSSSGSGS GS | 12 |
| linker | | |
| SEQ ID NO: 76 | SSSSSSGSGS GSGSGS | 16 |
| linker | | |

In an embodiment, a 4-1BB agonist fusion protein according to structures I-A or I-B comprises one or more 4-1BB binding domains selected from the group consisting of a variable heavy chain and variable light chain of utomilumab, a variable heavy chain and variable light chain of urelumab, a variable heavy chain and variable light chain of utomilumab, a variable heavy chain and variable light chain selected from the variable heavy chains and variable light chains described in Table 10, any combination of a variable heavy chain and variable light chain of the foregoing, and fragments, derivatives, conjugates, variants, and biosimilars thereof.

In an embodiment, a 4-1BB agonist fusion protein according to structures I-A or I-B comprises one or more 4-1BB binding domains comprising a 4-1BBL sequence. In an embodiment, a 4-1BB agonist fusion protein according to structures I-A or I-B comprises one or more 4-1BB binding domains comprising a sequence according to SEQ ID NO:77. In an embodiment, a 4-1BB agonist fusion protein according to structures I-A or I-B comprises one or more 4-1BB binding domains comprising a soluble 4-1BBL sequence. In an embodiment, a 4-1BB agonist fusion protein according to structures I-A or I-B comprises one or more 4-1BB binding domains comprising a sequence according to SEQ ID NO:78.

In an embodiment, a 4-1BB agonist fusion protein according to structures I-A or I-B comprises one or more 4-1BB binding domains that is a scFv domain comprising V_{H} and V_{L} regions that are each at least 95% identical to the sequences shown in SEQ ID NO:43 and SEQ ID NO:44, respectively, wherein the V_{H} and V_{L} domains are connected by a linker. In an embodiment, a 4-1BB agonist fusion protein according to structures I-A or I-B comprises one or more 4-1BB binding domains that is a scFv domain comprising V_{H} and V_{L} regions that are each at least 95% identical to the sequences shown in SEQ ID NO:54 and SEQ ID NO:55, respectively, wherein the V_{H} and V_{L} domains are connected by a linker. In an embodiment, a 4-1BB agonist fusion protein according to structures I-A or I-B comprises one or more 4-1BB binding domains that is a scFv domain comprising V_{H} and V_{L} regions that are each at least 95% identical to the V_{H} and V_{L} sequences given in Table 10, wherein the V_{H} and V_{L} domains are connected by a linker.

TABLE 10
Additional polypeptide domains useful as 4-1BB binding domains in fusion
proteins or as scFv 4-1BB agonist antibodies.

| Identifier | Sequence (One-Letter Amino Acid Symbols) | |
|---|---|---|
| SEQ ID NO: 77 | MEYASDASLD PEAPWPPAPR ARACRVLPWA LVAGLLLLLL LAAACAVFLA CPWAVSGARA | 60 |
| 4 - 1BBL | SPGSAASPRL REGPELSPDD PAGLLDLRQG MFAQLVAQNV LLIDGPLSWY SDPGLAGVSL | 120 |
| | TGGLSYKEDT KELVVAKAGV YYVFFQLELR RVVAGEGSGS VSLALHLQPL RSAAGAAALA | 180 |
| | LTVDLPPASS EARNSAFGFQ GRLLHLSAGQ RLGVHLHTEA RARHAWQLTQ GATVLGLFRV | 240 |
| | TPEIPAGLPS PRSE | 254 |
| SEQ ID NO: 78 | LRQGMFAQLV AQNVLLIDGP LSWYSDPGLA GVSLTGGLSY KEDTKELVVA KAGVYYVFFQ | 60 |
| 4 -1BBL soluble | LELRRVVAGE GSGSVSLALH LQPLRSAAGA AALALTVDLP PASSEARNSA FGFQGRLLHL | 120 |
| domain | SAGQRLGVHL HTEARARHAW QLTQGATVLG LFRVTPEIPA GLPSPRSE | 168 |
| SEQ ID NO: 79 | QVQLQQPGAE LVKPGASVKL SCKASGYTFS SYWMHWVKQR PGQVLEWIGE INPGNGHTNY | 60 |
| variable heavy | NEKFKSKATL TVDKSSSTAY MQLSSLTSED SAVYYCARSF TTARGFAYWG QGTLVTVS | 118 |
| chain for 4B4-1- | | |
| 1 version 1 | | |
| SEQ ID NO: 80 | DIVMTQSPAT QSVTPGDRVS LSCRASQTIS DYLHWYQQKS HESPRLLIKY ASQSISGIPS | 60 |
| variable light | RFSGSGSGSD FTLSINSVEP EDVGVYYCQD GHSFPPTFGG GTKLEIK | 107 |
| chain for 4B4-1- | | |
| 1 version 1 | | |
| SEQ ID NO: 81 | QVQLQQPGAE LVKPGASVKL SCKASGYTFS SYWMHWVKQR PGQVLEWIGE INPGNGHTNY | 60 |
| variable heavy | NEKFKSKATL TVDKSSSTAY MQLSSLTSED SAVYYCARSF TTARGFAYWG QGTLVTVSA | 119 |
| chain for 4B4-1- | | |
| 1 version 2 | | |
| SEQ ID NO: 82 | DIVMTQSPAT QSVTPGDRVS LSCRASQTIS DYLHWYQQKS HESPRLLIKY ASQSISGIPS | 60 |
| variable light | RFSGSGSGSD FTLSINSVEP EDVGVYYCQD GHSFPPTFGG GTKLEIKR | 108 |
| chain for 4B4-1- | | |
| 1 version 2 | | |
| SEQ ID NO: 83 | MDWTWRILFL VAAATGAHSE VQLVESGGGL VQPGGSLRLS CAASGFTFSD YWMSWVRQAP | 60 |
| variable heavy | GKGLEWVADI KNDGSYTNYA PSLTNRFTIS RDNAKNSLYL QMNSLRAEDT AVYYCARELT | 120 |
| chain for H39E3 - | | |
| 2 | | |
| SEQ ID NO: 84 | MEAPAQLLFL LLLWLPDTTG DIVMTQSPDS LAVSLGERAT INCKSSQSLL SSGNQKNYL | 60 |
| variable light | WYQQKPGQPP KLLIYYASTR QSGVPDRFSG SGSGTDFTLT ISSLQAEDVA | 110 |
| chain for H39E3- | | |
| 2 | | |

In an embodiment, the 4-1BB agonist is a 4-1BB agonistic single-chain fusion polypeptide comprising (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, and wherein the additional domain is a Fab or Fc fragment domain. In an embodiment, the 4-1BB agonist is a 4-1BB agonistic single-chain fusion polypeptide comprising (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, wherein the additional domain is a Fab or Fc fragment domain, wherein each of the soluble 4-1BB domains lacks a stalk region (which contributes to trimerization and provides a certain distance to the cell membrane, but is not part of the 4-1BB binding domain) and the first and the second peptide linkers independently have a length of 3-8 amino acids.

In an embodiment, the 4-1BB agonist is a 4-1BB agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein each TNF superfamily cytokine domain is a 4-1BB binding domain.

In an embodiment, the 4-1BB agonist is a 4-1BB agonistic scFv antibody comprising any of the foregoing V_{H} domains linked to any of the foregoing V_{L} domains.

In an embodiment, the 4-1BB agonist is BPS Bioscience 4-1BB agonist antibody catalog no. 79097-2, commercially available from BPS Bioscience, San Diego, CA, USA. In an embodiment, the 4-1BB agonist is Creative Biolabs 4-1BB agonist antibody catalog no. MOM-18179, commercially available from Creative Biolabs, Shirley, NY, USA.

### 3. OX40 (CD134) Agonists

In an embodiment, the TNFRSF agonist is an OX40 (CD134) agonist. The OX40 agonist may be any OX40 binding molecule known in the art. The OX40 binding molecule may be a monoclonal antibody or fusion protein capable of binding to human or mammalian OX40. The OX40 agonists or OX40 binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The OX40 agonist or OX40 binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to OX40. In an embodiment, the OX40 agonist is an antigen binding protein that is a fully human antibody. In an embodiment, the OX40 agonist is an antigen binding protein that is a humanized antibody. In some embodiments, OX40 agonists for use in the presently disclosed methods and compositions include anti-OX40 antibodies, human anti-OX40 antibodies, mouse anti-OX40 antibodies, mammalian anti-OX40 antibodies, monoclonal anti-OX40 antibodies, polyclonal anti-OX40 antibodies, chimeric anti-OX40 antibodies, anti-OX40 adnectins, anti-OX40 domain antibodies, single chain anti-OX40 fragments, heavy chain anti-OX40 fragments, light chain anti-OX40 fragments, anti-OX40 fusion proteins, and fragments, derivatives, conjugates, variants, or biosimilars thereof. In a preferred embodiment, the OX40 agonist is an agonistic, anti-OX40 humanized or fully human monoclonal antibody (i.e., an antibody derived from a single cell line).

In a preferred embodiment, the OX40 agonist or OX40 binding molecule may also be a fusion protein. OX40 fusion proteins comprising an Fc domain fused to OX40L are described, for example, in Sadun, et al., J. Immunother. 2009, 182, 1481-89. In a preferred embodiment, a multimeric OX40 agonist, such as a trimeric or hexameric OX40 agonist (with three or six ligand binding domains), may induce superior receptor (OX40L) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic OX40 antibodies and fusion proteins are known to induce strong immune responses. Curti, et al., Cancer Res. 2013, 73, 7189-98. In a preferred embodiment, the OX40 agonist is a monoclonal antibody or fusion protein that binds specifically to OX40 antigen in a manner sufficient to reduce toxicity. In some embodiments, the OX40 agonist is an agonistic OX40 monoclonal antibody or fusion protein that abrogates antibody-dependent cellular toxicity (ADCC), for example NK cell cytotoxicity. In some embodiments, the OX40 agonist is an agonistic OX40 monoclonal antibody or fusion protein that abrogates antibody-dependent cell phagocytosis (ADCP). In some embodiments, the OX40 agonist is an agonistic OX40 monoclonal antibody or fusion protein that abrogates complement-dependent cytotoxicity (CDC). In some embodiments, the OX40 agonist is an agonistic OX40 monoclonal antibody or fusion protein which abrogates Fc region functionality.

In some embodiments, the OX40 agonists are characterized by binding to human OX40 (SEQ ID NO:85) with high affinity and agonistic activity. In an embodiment, the OX40 agonist is a binding molecule that binds to human OX40 (SEQ ID NO:85). In an embodiment, the OX40 agonist is a binding molecule that binds to murine OX40 (SEQ ID NO:86). The amino acid sequences of OX40 antigen to which an OX40 agonist or binding molecule binds are summarized in Table 11.

TABLE 11
Amino acid sequences of OX40 antigens.

| Identifier | Sequence (One-Letter Amino Acid Symbols) | |
|---|---|---|
| SEQ ID NO: 85 | MCVGARRLGR GPCAALLLLG LGLSTVTGLE CVGDTYPSND RCCHECRPGN GMVSRCSRSQ | 60 |
| human OX40 | NTVCRPCGPG FYNDVVSSKP CKPCTWCNLR SGSERKQLCT ATQDTVCRCR AGTQPLDSYK | 120 |
| (Homo sapiens) | PGVDCAPCPP GHFSPGDNQA CKPWTNCTLA GKHTLQPASN SSDAICEDRD PPATQPQETQ | 180 |
| | GPPARPITVQ PTEAWPRTSQ GPSTRPVEVP GGRAVAAILG LGLVLGLLGP LAILLALYLL | 240 |
| | RRDQRLPPDA HKPPGGGSFR TPIQEEQADA HSTLAKI | 277 |
| SEQ ID NO: 86 | MYVWVQQPTA LLLLGLTLGV TARRLNCVKE TYPSGHKCCR ECQPGHGMVS RCDHTRDTLC | 60 |
| murine OX40 | EPCETGFYNE AVNYDTCKQC TQCNHRSGSE LKQNCTPTQD TVCRCRPGTQ PRQDSGYKLG | 120 |
| (Mus musculus) | VDCVPCPPGH FSPGNNQACK PWTNCTLSGK QTRHPASDSL DAVCEDRSLL ATLLWETQRP | 180 |
| | TFRPTTVQST TVWPRTSELP SPPTLVTPEG PAFAVLLGLG LGLLAPLTVL LALYLLRKAW | 240 |
| | RLPNTPKPCW GNSFRTPIQE EHTDAHFTLA KI | 272 |

In some embodiments, the compositions, processes and methods described include a OX40 agonist that binds human or murine OX40 with a K_{D} of about 100 pM or lower, binds human or murine OX40 with a K_{D} of about 90 pM or lower, binds human or murine OX40 with a K_{D} of about 80 pM or lower, binds human or murine OX40 with a K_{D} of about 70 pM or lower, binds human or murine OX40 with a K_{D} of about 60 pM or lower, binds human or murine OX40 with a K_{D} of about 50 pM or lower, binds human or murine OX40 with a K_{D} of about 40 pM or lower, or binds human or murine OX40 with a K_{D} of about 30 pM or lower.

In some embodiments, the compositions, processes and methods described include a OX40 agonist that binds to human or murine OX40 with a k_{assoc} of about 7.5×10⁵ l/M·s or faster, binds to human or murine OX40 with a k_{assoc} of about 7.5×10⁵ l/M·s or faster, binds to human or murine OX40 with a k_{assoc} of about 8×10⁵ l/M·s or faster, binds to human or murine OX40 with a k_{assoc} of about 8.5×10⁵ l/M·s or faster, binds to human or murine OX40 with a k_{assoc} of about 9×10⁵ l/M·s or faster, binds to human or murine OX40 with a k_{assoc} of about 9.5×10⁵ l/M·s or faster, or binds to human or murine OX40 with a k_{assoc} of about 1×10⁶ l/M·s or faster.

In some embodiments, the compositions, processes and methods described include a OX40 agonist that binds to human or murine OX40 with a k_{dissoc} of about 2×10⁻⁵ l/s or slower, binds to human or murine OX40 with a k_{dissoc} of about 2.1×10⁻⁵ l/s or slower, binds to human or murine OX40 with a k_{dissoc} of about 2.2×10⁻⁵ l/s or slower, binds to human or murine OX40 with a k_{dissoc} of about 2.3×10⁻⁵ l/s or slower, binds to human or murine OX40 with a k_{dissoc} of about 2.4×10⁻⁵ l/s or slower, binds to human or murine OX40 with a k_{dissoc} of about 2.5×10⁻⁵ l/s or slower, binds to human or murine OX40 with a k_{dissoc} of about 2.6×10⁻⁵ l/s or slower or binds to human or murine OX40 with a k_{dissoc} of about 2.7×10⁻⁵ l/s or slower, binds to human or murine OX40 with a k_{dissoc} of about 2.8×10⁻⁵ l/s or slower, binds to human or murine OX40 with a k_{dissoc} of about 2.9×10⁻⁵ l/s or slower, or binds to human or murine OX40 with a k_{dissoc} of about 3×10⁻⁵ l/s or slower.

In some embodiments, the compositions, processes and methods described include OX40 agonist that binds to human or murine OX40 with an IC₅₀ of about 10 nM or lower, binds to human or murine OX40 with an IC₅₀ of about 9 nM or lower, binds to human or murine OX40 with an IC₅₀ of about 8 nM or lower, binds to human or murine OX40 with an IC₅₀ of about 7 nM or lower, binds to human or murine OX40 with an IC₅₀ of about 6 nM or lower, binds to human or murine OX40 with an IC₅₀ of about 5 nM or lower, binds to human or murine OX40 with an IC₅₀ of about 4 nM or lower, binds to human or murine OX40 with an IC₅₀ of about 3 nM or lower, binds to human or murine OX40 with an IC₅₀ of about 2 nM or lower, or binds to human or murine OX40 with an IC₅₀ of about 1 nM or lower.

In some embodiments, the OX40 agonist is tavolixizumab, also known as MEDI0562 or MEDI-0562. Tavolixizumab is available from the MedImmune subsidiary of AstraZeneca, Inc. Tavolixizumab is immunoglobulin G1-kappa, anti-[Homo sapiens TNFRSF4 (tumor necrosis factor receptor (TNFR) superfamily member 4, OX40, CD134)], humanized and chimeric monoclonal antibody. The amino acid sequences of tavolixizumab are set forth in Table 12. Tavolixizumab comprises N-glycosylation sites at positions 301 and 301″, with fucosylated complex bi-antennary CHO-type glycans; heavy chain intrachain disulfide bridges at positions 22-95 (V_{H}-V_{L}), 148-204 (C_{H}1-C_{L}), 265-325 (C_{H}2) and 371-429 (C_{H}3) (and at positions 22″-95″, 148″-204″, 265″-325″, and 371″-429″); light chain intrachain disulfide bridges at positions 23′-88′ (V_{H}-V_{L}) and 134′-194′ (C_{H}1-C_{L}) (and at positions 23′″-88′″ and 134′″-194′″); interchain heavy chain-heavy chain disulfide bridges at positions 230-230″ and 233-233″; and interchain heavy chain-light chain disulfide bridges at 224-214′ and 224″-214′″. Current clinical trials of tavolixizumab in a variety of solid tumor indications include U.S. National Institutes of Health clinicaltrials.gov identifiers NCT02318394 and NCT02705482.

In an embodiment, a OX40 agonist comprises a heavy chain given by SEQ ID NO:87 and a light chain given by SEQ ID NO:88. In an embodiment, a OX40 agonist comprises heavy and light chains having the sequences shown in SEQ ID NO:87 and SEQ ID NO:88, respectively, or antigen binding fragments, Fab fragments, single-chain variable fragments (scFv), variants, or conjugates thereof. In an embodiment, a OX40 agonist comprises heavy and light chains that are each at least 99% identical to the sequences shown in SEQ ID NO:87 and SEQ ID NO:88, respectively. In an embodiment, a OX40 agonist comprises heavy and light chains that are each at least 98% identical to the sequences shown in SEQ ID NO:87 and SEQ ID NO:88, respectively. In an embodiment, a OX40 agonist comprises heavy and light chains that are each at least 97% identical to the sequences shown in SEQ ID NO:87 and SEQ ID NO:88, respectively. In an embodiment, a OX40 agonist comprises heavy and light chains that are each at least 96% identical to the sequences shown in SEQ ID NO:87 and SEQ ID NO:88, respectively. In an embodiment, a OX40 agonist comprises heavy and light chains that are each at least 95% identical to the sequences shown in SEQ ID NO:87 and SEQ ID NO:88, respectively.

In an embodiment, the OX40 agonist comprises the heavy and light chain CDRs or variable regions (VRs) of tavolixizumab. In an embodiment, the OX40 agonist heavy chain variable region (V_{H}) comprises the sequence shown in SEQ ID NO:89, and the OX40 agonist light chain variable region (V_{L}) comprises the sequence shown in SEQ ID NO:90, and conservative amino acid substitutions thereof. In an embodiment, a OX40 agonist comprises V_{H} and V_{L} regions that are each at least 99% identical to the sequences shown in SEQ ID NO:89 and SEQ ID NO:90, respectively. In an embodiment, a OX40 agonist comprises V_{H} and V_{L} regions that are each at least 98% identical to the sequences shown in SEQ ID NO:89 and SEQ ID NO:90, respectively. In an embodiment, a OX40 agonist comprises V_{H} and V_{L} regions that are each at least 97% identical to the sequences shown in SEQ ID NO:89 and SEQ ID NO:90, respectively. In an embodiment, a OX40 agonist comprises V_{H} and V_{L} regions that are each at least 96% identical to the sequences shown in SEQ ID NO:89 and SEQ ID NO:90, respectively. In an embodiment, a OX40 agonist comprises V_{H} and V_{L} regions that are each at least 95% identical to the sequences shown in SEQ ID NO:89 and SEQ ID NO:90, respectively. In an embodiment, an OX40 agonist comprises an scFv antibody comprising V_{H} and V_{L} regions that are each at least 99% identical to the sequences shown in SEQ ID NO:89 and SEQ ID NO:90.

In an embodiment, a OX40 agonist comprises heavy chain CDR1, CDR2 and CDR3 domains having the sequences set forth in SEQ ID NO:91, SEQ ID NO:92, and SEQ ID NO:93, respectively, and conservative amino acid substitutions thereof, and light chain CDR1, CDR2 and CDR3 domains having the sequences set forth in SEQ ID NO:94, SEQ ID NO:95, and SEQ ID NO:96, respectively, and conservative amino acid substitutions thereof.

In an embodiment, the OX40 agonist is a OX40 agonist biosimilar monoclonal antibody approved by drug regulatory authorities with reference to tavolixizumab. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tavolixizumab. In some embodiments, the one or more post-translational modifications are selected from one or more of glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is tavolixizumab. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tavolixizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tavolixizumab.

TABLE 12
Amino acid sequences for OX40 agonist antibodies related to tavolixizumab.

| Identifier | Sequence (One-Letter Amino Acid Symbols) | |
|---|---|---|
| SEQ ID NO: 87 | QVQLQESGPG LVKPSQTLSL TCAVYGGSFS SGYWNWIRKH PGKGLEYIGY ISYNGITYHN | 60 |
| heavy chain for | PSLKSRITIN RDTSKNQYSL QLNSVTPEDT AVYYCARYKY DYDGGHAMDY WGQGTLVTVS | 120 |
| tavolixizumab | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPELLG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPAPIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 88 | DIQMTQSPSS LSASVGDRVT ITCRASQDIS NYLNWYQQKP GKAPKLLIYY TSKLHSGVPS | 60 |
| light chain for | RFSGSGSGTD YTLTISSLQP EDFATYYCQQ GSALPWTFGQ GTKVEIKRTV AAPSVFIFPP | 120 |
| tavolixizumab | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 89 | QVQLQESGPG LVKPSQTLSL TCAVYGGSFS SGYWNWIRKH PGKGLEYIGY ISYNGITYHN | 60 |
| heavy chain | PSLKSRITIN RDTSKNQYSL QLNSVTPEDT AVYYCARYKY DYDGGHAMDY WGQGTLVT | 118 |
| variable region | | |
| for | | |
| tavolixizumab | | |
| SEQ ID NO: 90 | DIQMTQSPSS LSASVGDRVT ITCRASQDIS NYLNWYQQKP GKAPKLLIYY TSKIHSGVPS | 60 |
| light chain | RFSGSGSGTD YTLTISSLQP EDFATYYCQQ GSALPWTFGQ GTKVEIKR | 108 |
| variable region | | |
| for | | |
| tavolixizumab | | |
| SEQ ID NO: 91 | GSFSSGYWN | 9 |
| heavy chain CDR1 | | |
| for | | |
| tavolixizumab | | |
| SEQ ID NO: 92 | YIGYISYNGI TYH | 13 |
| heavy chain CDR2 | | |
| for | | |
| tavolixizumab | | |
| SEQ ID NO: 93 | RYKYDYDGGH AMDY | 14 |
| heavy chain CDR3 | | |
| for | | |
| tavolixizumab | | |
| SEQ ID NO: 94 | QDISNYLN | 8 |
| light chain CDR1 | | |
| for | | |
| tavolixizumab | | |
| SEQ ID NO: 95 | LLIYYTSKLH S | 11 |
| light chain CDR2 | | |
| for | | |
| tavolixizumab | | |
| SEQ ID NO: 96 | QQGSALPW | 8 |
| light chain CDR3 | | |
| for | | |
| tavolixizumab | | |

In some embodiments, the OX40 agonist is 11D4, which is a fully human antibody available from Pfizer, Inc. The preparation and properties of 11D4 are described in U.S. Pat. Nos. 7,960,515; 8,236,930; and 9,028,824, the disclosures of which are incorporated by reference herein. The amino acid sequences of 11D4 are set forth in Table 13.

In an embodiment, a OX40 agonist comprises a heavy chain given by SEQ ID NO:97 and a light chain given by SEQ ID NO:98. In an embodiment, a OX40 agonist comprises heavy and light chains having the sequences shown in SEQ ID NO:97 and SEQ ID NO:98, respectively, or antigen binding fragments, Fab fragments, single-chain variable fragments (scFv), variants, or conjugates thereof. In an embodiment, a OX40 agonist comprises heavy and light chains that are each at least 99% identical to the sequences shown in SEQ ID NO:97 and SEQ ID NO:98, respectively. In an embodiment, a OX40 agonist comprises heavy and light chains that are each at least 98% identical to the sequences shown in SEQ ID NO:97 and SEQ ID NO:98, respectively. In an embodiment, a OX40 agonist comprises heavy and light chains that are each at least 97% identical to the sequences shown in SEQ ID NO:97 and SEQ ID NO:98, respectively. In an embodiment, a OX40 agonist comprises heavy and light chains that are each at least 96% identical to the sequences shown in SEQ ID NO:97 and SEQ ID NO:98, respectively. In an embodiment, a OX40 agonist comprises heavy and light chains that are each at least 95% identical to the sequences shown in SEQ ID NO:97 and SEQ ID NO:98, respectively.

In an embodiment, the OX40 agonist comprises the heavy and light chain CDRs or variable regions (VRs) of 11D4. In an embodiment, the OX40 agonist heavy chain variable region (VH) comprises the sequence shown in SEQ ID NO:99, and the OX40 agonist light chain variable region (VL) comprises the sequence shown in SEQ ID NO:100, and conservative amino acid substitutions thereof. In an embodiment, a OX40 agonist comprises VH and VL regions that are each at least 99% identical to the sequences shown in SEQ ID NO:99 and SEQ ID NO:100, respectively. In an embodiment, a OX40 agonist comprises VH and VL regions that are each at least 98% identical to the sequences shown in SEQ ID NO:99 and SEQ ID NO:100, respectively. In an embodiment, a OX40 agonist comprises VH and VL regions that are each at least 97% identical to the sequences shown in SEQ ID NO:99 and SEQ ID NO:100, respectively. In an embodiment, a OX40 agonist comprises VH and VL regions that are each at least 96% identical to the sequences shown in SEQ ID NO:99 and SEQ ID NO:100, respectively. In an embodiment, a OX40 agonist comprises VH and VL regions that are each at least 95% identical to the sequences shown in SEQ ID NO:99 and SEQ ID NO:100, respectively.

In an embodiment, a OX40 agonist comprises heavy chain CDR1, CDR2 and CDR3 domains having the sequences set forth in SEQ ID NO:101, SEQ ID NO:102, and SEQ ID NO:103, respectively, and conservative amino acid substitutions thereof, and light chain CDR1, CDR2 and CDR3 domains having the sequences set forth in SEQ ID NO:104, SEQ ID NO:105, and SEQ ID NO:106, respectively, and conservative amino acid substitutions thereof.

In an embodiment, the OX40 agonist is a OX40 agonist biosimilar monoclonal antibody approved by drug regulatory authorities with reference to 11D4. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 11D4. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 11D4. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 11D4. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 11D4.

TABLE 13
Amino acid sequences for OX40 agonist antibodies related to 11D4.

| Identifier | Sequence (One-Letter Amino Acid Symbols) | |
|---|---|---|
| SEQ ID NO: 97 | EVQLVESGGG LVQPGGSLRL SCAASGFTFS SYSMNWVRQA PGKGLEWVSY ISSSSSTIDY | 60 |
| heavy chain for | ADSVKGRFTI SRDNAKNSLY LQMNSLRDED TAVYYCARES GWYLFDYWGQ GTLVTVSSAS | 120 |
| 11D4 | TKGPSVFPLA PCSRSTSEST AALGCLVKDY FPEPVTVSWN SGALTSGVHT FPAVLQSSGL | 180 |
| | YSLSSVVTVP SSNFGTQTYT CNVDHKPSNT KVDKTVERKC CVECPPCPAP PVAGPSVFLF | 240 |
| | PPKPKDTLMI SRTPEVTCVV VDVSHEDPEV QFNWYVDGVE VHNAKTKPRE EQFNSTFRVV | 300 |
| | SVLTVVHQDW LNGKEYKCKV SNKGLPAPIE KTISKTKGQP REPQVYTLPP SREEMTKNQV | 360 |
| | SLTCLVKGFY PSDIAVEWES NGQPENNYKT TPPMLDSDGS FFLYSKLTVD KSRWQQGNVF | 420 |
| | SCSVMHEALH NHYTQKSLSL SPGK | 444 |
| SEQ ID NO: 98 | DIQMTQSPSS LSASVGDRVT ITCRASQGIS SWLAWYQQKP EKAPKSLIYA ASSLQSGVPS | 60 |
| light chain for | RFSGSGSGTD FTLTISSLQP EDFATYYCQQ YNSYPPTFGG GTKVEIKRTV AAPSVFIFPP | 120 |
| 11D4 | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 99 | EVQLVESGGG LVQPGGSLRL SCAASGFTFS SYSMNWVRQA PGKGLEWVSY ISSSSSTIDY | 60 |
| heavy chain | ADSVKGRFTI SRDNAKNSLY LQMNSLRDED TAVYYCARES GWYLFDYWGQ GTLVTVSS | 118 |
| variable region | | |
| for 11D4 | | |
| SEQ ID NO: 100 | DIQMTQSPSS LSASVGDRVT ITCRASQGIS SWLAWYQQKP EKAPKSLIYA ASSLQSGVPS | 60 |
| light chain | RFSGSGSGTD FTLTISSLQP EDFATYYCQQ YNSYPPTFGG GTKVEIK | 107 |
| variable region | | |
| for 11D4 | | |
| SEQ ID NO: 101 | SYSMN | 5 |
| heavy chain CDR1 | | |
| for 11D4 | | |
| SEQ ID NO: 102 | YISSSSSTID YADSVKG | 17 |
| heavy chain CDR2 | | |
| for 11D4 | | |
| SEQ ID NO: 103 | ESGWYLFDY | 9 |
| heavy chain CDR3 | | |
| for 11D4 | | |
| SEQ ID NO: 104 | RASQGISSWL A | 11 |
| light chain CDR1 | | |
| for 11D4 | | |
| SEQ ID NO: 105 | AASSLQS | 7 |
| light chain CDR2 | | |
| for 11D4 | | |
| SEQ ID NO: 106 | QQYNSYPPT | 9 |
| light chain CDR3 | | |
| for 11D4 | | |

In some embodiments, the OX40 agonist is 18D8, which is a fully human antibody available from Pfizer, Inc. The preparation and properties of 18D8 are described in U.S. Pat. Nos. 7,960,515; 8,236,930; and 9,028,824, the disclosures of which are incorporated by reference herein. The amino acid sequences of 18D8 are set forth in Table 14.

In an embodiment, a OX40 agonist comprises a heavy chain given by SEQ ID NO:107 and a light chain given by SEQ ID NO:108. In an embodiment, a OX40 agonist comprises heavy and light chains having the sequences shown in SEQ ID NO:107 and SEQ ID NO:108, respectively, or antigen binding fragments, Fab fragments, single-chain variable fragments (scFv), variants, or conjugates thereof. In an embodiment, a OX40 agonist comprises heavy and light chains that are each at least 99% identical to the sequences shown in SEQ ID NO:107 and SEQ ID NO:108, respectively. In an embodiment, a OX40 agonist comprises heavy and light chains that are each at least 98% identical to the sequences shown in SEQ ID NO:107 and SEQ ID NO:108, respectively. In an embodiment, a OX40 agonist comprises heavy and light chains that are each at least 97% identical to the sequences shown in SEQ ID NO:107 and SEQ ID NO:108, respectively. In an embodiment, a OX40 agonist comprises heavy and light chains that are each at least 96% identical to the sequences shown in SEQ ID NO:107 and SEQ ID NO:108, respectively. In an embodiment, a OX40 agonist comprises heavy and light chains that are each at least 95% identical to the sequences shown in SEQ ID NO:107 and SEQ ID NO:108, respectively.

In an embodiment, the OX40 agonist comprises the heavy and light chain CDRs or variable regions (VRs) of 18D8. In an embodiment, the OX40 agonist heavy chain variable region (V_{H}) comprises the sequence shown in SEQ ID NO:109, and the OX40 agonist light chain variable region (V_{L}) comprises the sequence shown in SEQ ID NO:110, and conservative amino acid substitutions thereof. In an embodiment, a OX40 agonist comprises V_{H} and V_{L} regions that are each at least 99% identical to the sequences shown in SEQ ID NO:109 and SEQ ID NO:110, respectively. In an embodiment, a OX40 agonist comprises V_{H} and V_{L} regions that are each at least 98% identical to the sequences shown in SEQ ID NO:109 and SEQ ID NO:110, respectively. In an embodiment, a OX40 agonist comprises V_{H} and V_{L} regions that are each at least 97% identical to the sequences shown in SEQ ID NO:109 and SEQ ID NO:110, respectively. In an embodiment, a OX40 agonist comprises V_{H} and V_{L} regions that are each at least 96% identical to the sequences shown in SEQ ID NO:109 and SEQ ID NO:110, respectively. In an embodiment, a OX40 agonist comprises V_{H} and V_{L} regions that are each at least 95% identical to the sequences shown in SEQ ID NO:109 and SEQ ID NO:110, respectively.

In an embodiment, a OX40 agonist comprises heavy chain CDR1, CDR2 and CDR3 domains having the sequences set forth in SEQ ID NO:111, SEQ ID NO:112, and SEQ ID NO:113, respectively, and conservative amino acid substitutions thereof, and light chain CDR1, CDR2 and CDR3 domains having the sequences set forth in SEQ ID NO:114, SEQ ID NO:115, and SEQ ID NO:116, respectively, and conservative amino acid substitutions thereof.

In an embodiment, the OX40 agonist is a OX40 agonist biosimilar monoclonal antibody approved by drug regulatory authorities with reference to 18D8. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 18D8. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 18D8. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 18D8. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 18D8.

TABLE 14
Amino acid sequences for OX40 agonist antibodies related to 18D8.

| Identifier | Sequence (One-Letter Amino Acid Symbols) | |
|---|---|---|
| SEQ ID NO: 107 | EVQLVESGGG LVQPGRSLRL SCAASGFTFD DYAMHWVRQA PGKGLEWVSG ISWNSGSIGY | 60 |
| heavy chain for | ADSVKGRFTI SRDNAKNSLY LQMNSLRAED TALYYCAKDQ STADYYFYYG MDVWGQGTTV | 120 |
| 18D8 | TVSSASTKGP SVFPLAPCSR STSESTAALG CLVKDYFPEP VTVSWNSGAL TSGVHTFPAV | 180 |
| | LQSSGLYSLS SVVTVPSSNF GTQTYTCNVD HKPSNTKVDK TVERKCCVEC PPCPAPPVAG | 240 |
| | PSVFLFPPKP KDTLMISRTP EVTCVVVDVS HEDPEVQFNW YVDGVEVHNA KTKPREEQFN | 300 |
| | STFRVVSVLT VVHQDWLNGK EYKCKVSNKG LPAPIEKTIS KTKGQPREPQ VYTIPPSREE | 360 |
| | MTKNQVSLTC LVKGFYPSDI AVEWESNGQP ENNYKTTPPM LDSDGSFFLY SKLTVDKSRW | 420 |
| | QQGNVFSCSV MHEALHNHYT QKSLSLSPGK | 450 |
| SEQ ID NO: 108 | EIVVTQSPAT LSLSPGERAT LSCRASQSVS SYLAWYQQKP GQAPRLLIYD ASNRATGIPA | 60 |
| light chain for | RFSGSGSGTD FTLTISSLEP EDFAVYYCQQ RSNWPTFGQG TKVEIKRTVA APSVFIFPPS | 120 |
| 18D8 | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 109 | EVQLVESGGG LVQPGRSLRL SCAASGFTFD DYAMHWVRQA PGKGLEWVSG ISWNSGSIGY | 60 |
| heavy chain | ADSVKGRFTI SRDNAKNSLY LQMNSLRAED TALYYCAKDQ STADYYFYYG MDVWGQGTTV | 120 |
| variable region | TVSS | 124 |
| for 18D8 | | |
| SEQ ID NO: 110 | EIVVTQSPAT LSLSPGERAT LSCRASQSVS SYLAWYQQKP GQAPRLLIYD ASNRATGIPA | 60 |
| light chain | RFSGSGSGTD FTLTISSLEP EDFAVYYCQQ RSNWPTFGQG TKVEIK | 106 |
| variable region | | |
| for 18D8 | | |
| SEQ ID NO: 111 | DYAMH | 5 |
| heavy chain CDR1 | | |
| for 18D8 | | |
| SEQ ID NO: 112 | GISWNSGSIG YADSVKG | 17 |
| heavy chain CDR2 | | |
| for 18D8 | | |
| SEQ ID NO: 113 | DQSTADYYFY YGMDV | 15 |
| heavy chain CDR3 | | |
| for 18D8 | | |
| SEQ ID NO: 114 | RASQSVSSYL A | 11 |
| light chain CDR1 | | |
| for 18D8 | | |
| SEQ ID NO: 115 | DASNRAT | 7 |
| light chain CDR2 | | |
| for 18D8 | | |
| SEQ ID NO: 116 | QQRSNWPT | 8 |
| light chain CDR3 | | |
| for 18D8 | | |

In some embodiments, the OX40 agonist is Hu119-122, which is a humanized antibody available from GlaxoSmithKline plc. The preparation and properties of Hu119-122 are described in U.S. Pat. Nos. 9,006,399 and 9,163,085, and in International Patent Publication No. WO 2012/027328, the disclosures of which are incorporated by reference herein. The amino acid sequences of Hu119-122 are set forth in Table 15.

In an embodiment, the OX40 agonist comprises the heavy and light chain CDRs or variable regions (VRs) of Hu119-122. In an embodiment, the OX40 agonist heavy chain variable region (V_{H}) comprises the sequence shown in SEQ ID NO:117, and the OX40 agonist light chain variable region (V_{L}) comprises the sequence shown in SEQ ID NO:118, and conservative amino acid substitutions thereof. In an embodiment, a OX40 agonist comprises V_{H} and V_{L} regions that are each at least 99% identical to the sequences shown in SEQ ID NO:117 and SEQ ID NO:118, respectively. In an embodiment, a OX40 agonist comprises V_{H} and V_{L} regions that are each at least 98% identical to the sequences shown in SEQ ID NO:117 and SEQ ID NO:118, respectively. In an embodiment, a OX40 agonist comprises V_{H} and V_{L} regions that are each at least 97% identical to the sequences shown in SEQ ID NO:117 and SEQ ID NO:118, respectively. In an embodiment, a OX40 agonist comprises V_{H} and V_{L} regions that are each at least 96% identical to the sequences shown in SEQ ID NO:117 and SEQ ID NO:118, respectively. In an embodiment, a OX40 agonist comprises V_{H} and V_{L} regions that are each at least 95% identical to the sequences shown in SEQ ID NO:117 and SEQ ID NO:118, respectively.

In an embodiment, a OX40 agonist comprises heavy chain CDR1, CDR2 and CDR3 domains having the sequences set forth in SEQ ID NO:119, SEQ ID NO:120, and SEQ ID NO:121, respectively, and conservative amino acid substitutions thereof, and light chain CDR1, CDR2 and CDR3 domains having the sequences set forth in SEQ ID NO:122, SEQ ID NO:123, and SEQ ID NO:124, respectively, and conservative amino acid substitutions thereof.

In an embodiment, the OX40 agonist is a OX40 agonist biosimilar monoclonal antibody approved by drug regulatory authorities with reference to Hu119-122. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu119-122. In some embodiments, the one or more post-translational modifications are selected from one or more of glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu119-122. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu119-122. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu119-122.

TABLE 15
Amino acid sequences for OX40 agonist antibodies related to Hu119-122.

| Identifier | Sequence (One-Letter Amino Acid Symbols) | |
|---|---|---|
| SEQ ID NO: 117 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY LQMNSLRAED TAVYYCARHY DDYYAWFAYW GQGTMVTVSS | 120 |
| variable region | | |
| for Hul19-122 | | |
| SEQ ID NO: 118 | EIVLTQSPAT LSLSPGERAT LSCRASKSVS TSGYSYMHWY QQKPGQAPRL LIYLASNLES | 60 |
| light chain | GVPARFSGSG SGTDFTLTIS SLEPEDFAVY YCQHSRELPL TEGGGTKVEI K | 111 |
| variable region | | |
| for Hu119-122 | | |
| SEQ ID NO: 119 | SHDMS | 5 |
| heavy chain CDR1 | | |
| for Hu119-122 | | |
| SEQ ID NO: 120 | AINSDGGSTY YPDTMER | 17 |
| heavy chain CDR2 | | |
| for Hu119-122 | | |
| SEQ ID NO: 121 | EYDDYYAWFA Y | 11 |
| heavy chain CDR3 | | |
| for Hu119-122 | | |
| SEQ ID NO: 122 | RASKSVSTSG YSYMH | 15 |
| light chain CDR1 | | |
| for Hul19-122 | | |
| SEQ ID NO: 123 | LASNLES | 7 |
| light chain CDR2 | | |
| for Hu119-122 | | |
| SEQ ID NO: 124 | QHSRELPLT | 9 |
| light chain CDR3 | | |
| for Hu119-122 | | |

In some embodiments, the OX40 agonist is Hu106-222, which is a humanized antibody available from GlaxoSmithKline plc. The preparation and properties of Hu106-222 are described in U.S. Pat. Nos. 9,006,399 and 9,163,085, and in International Patent Publication No. WO 2012/027328, the disclosures of which are incorporated by reference herein. The amino acid sequences of Hu106-222 are set forth in Table 16.

In an embodiment, the OX40 agonist comprises the heavy and light chain CDRs or variable regions (VRs) of Hu106-222. In an embodiment, the OX40 agonist heavy chain variable region (V_{H}) comprises the sequence shown in SEQ ID NO:125, and the OX40 agonist light chain variable region (V_{L}) comprises the sequence shown in SEQ ID NO:126, and conservative amino acid substitutions thereof. In an embodiment, a OX40 agonist comprises V_{H} and V_{L} regions that are each at least 99% identical to the sequences shown in SEQ ID NO:125 and SEQ ID NO:126, respectively. In an embodiment, a OX40 agonist comprises V_{H} and V_{L} regions that are each at least 98% identical to the sequences shown in SEQ ID NO:125 and SEQ ID NO:126, respectively. In an embodiment, a OX40 agonist comprises V_{H} and V_{L} regions that are each at least 97% identical to the sequences shown in SEQ ID NO:125 and SEQ ID NO:126, respectively. In an embodiment, a OX40 agonist comprises V_{H} and V_{L} regions that are each at least 96% identical to the sequences shown in SEQ ID NO:125 and SEQ ID NO:126, respectively. In an embodiment, a OX40 agonist comprises V_{H} and V_{L} regions that are each at least 95% identical to the sequences shown in SEQ ID NO:125 and SEQ ID NO:126, respectively.

In an embodiment, a OX40 agonist comprises heavy chain CDR1, CDR2 and CDR3 domains having the sequences set forth in SEQ ID NO:127, SEQ ID NO:128, and SEQ ID NO:129, respectively, and conservative amino acid substitutions thereof, and light chain CDR1, CDR2 and CDR3 domains having the sequences set forth in SEQ ID NO:130, SEQ ID NO:131, and SEQ ID NO:132, respectively, and conservative amino acid substitutions thereof.

In an embodiment, the OX40 agonist is a OX40 agonist biosimilar monoclonal antibody approved by drug regulatory authorities with reference to Hu106-222. In an embodiment, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu106-222. In some embodiments, the one or more post-translational modifications are selected from one or more of glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu106-222. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222.

TABLE 16
Amino acid sequences for OX40 agonist antibodies related to Hu106-222.

| Identifier | Sequence (One-Letter Amino Acid Symbols) | |
|---|---|---|
| SEQ ID NO: 125 | QVQLVQSGSE LKKPGASVKV SCKASGYTFT DYSMHWVRQA PGQGLKWMGW INTETGEPTY | 60 |
| heavy chain | ADDFKGRFVF SLDTSVSTAY LQISSLKAED TAVYYCANPY YDYVSYYAMD YWGQGTTVTV | 120 |
| variable region | SS | 122 |
| for Hu106-222 | | |
| SEQ ID NO: 126 | DIQMTQSPSS LSASVGDRVT ITCKASQDVS TAVAWYQQKP GKAPKLLIYS ASYLYTGVPS | 60 |
| light chain | | |
| variable region | RFSGSGSGTD FTFTISSLQP EDIATYYCQQ HYSTPREFGQ GTKLEIK | 107 |
| for Hu106-222 | | |
| SEQ ID NO: 127 | DYSMH | 5 |
| heavy chain CDR1 | | |
| for Hul06-222 | | |
| SEQ ID NO: 128 | WINTETGEPT YADDFKG | 17 |
| heavy chain CDR2 | | |
| for Hu106-222 | | |
| SEQ ID NO: 129 | PYYDYVSYYA MDY | 13 |
| heavy chain CDR3 | | |
| for Hu106-222 | | |
| SEQ ID NO: 130 | KASQDVSTAV A | 11 |
| light chain CDR1 | | |
| for Hu106-222 | | |
| SEQ ID NO: 131 | SASYLYT | 7 |
| light chain CDR2 | | |
| for Hu106-222 | | |
| SEQ ID NO: 132 | QQHYSTPRT | 9 |
| light chain CDR3 | | |
| for Hu106-222 | | |

In some embodiments, the OX40 agonist antibody is MEDI6469 (also referred to as 9B12). MEDI6469 is a murine monoclonal antibody. Weinberg, et al., J. Immunother. 2006, 29, 575-585. In some embodiments the OX40 agonist is an antibody produced by the 9B12 hybridoma, deposited with Biovest Inc. (Malvern, MA, USA), as described in Weinberg, et al., J. Immunother. 2006, 29, 575-585, the disclosure of which is hereby incorporated by reference in its entirety. In some embodiments, the antibody comprises the CDR sequences of MEDI6469. In some embodiments, the antibody comprises a heavy chain variable region sequence and/or a light chain variable region sequence of MEDI6469.

In an embodiment, the OX40 agonist is L106 BD (Pharmingen Product #340420). In some embodiments, the OX40 agonist comprises the CDRs of antibody L106 (BD Pharmingen Product #340420). In some embodiments, the OX40 agonist comprises a heavy chain variable region sequence and/or a light chain variable region sequence of antibody L106 (BD Pharmingen Product #340420). In an embodiment, the OX40 agonist is ACT35 (Santa Cruz Biotechnology, Catalog #20073). In some embodiments, the OX40 agonist comprises the CDRs of antibody ACT35 (Santa Cruz Biotechnology, Catalog #20073). In some embodiments, the OX40 agonist comprises a heavy chain variable region sequence and/or a light chain variable region sequence of antibody ACT35 (Santa Cruz Biotechnology, Catalog #20073). In an embodiment, the OX40 agonist is the murine monoclonal antibody anti-mCD134/mOX40 (clone OX86), commercially available from InVivoMAb, BioXcell Inc, West Lebanon, NH.

In an embodiment, the OX40 agonist is selected from the OX40 agonists described in International Patent Application Publication Nos. WO 95/12673, WO 95/21925, WO 2006/121810, WO 2012/027328, WO 2013/028231, WO 2013/038191, and WO 2014/148895; European Patent Application EP 0672141; U.S. Patent Application Publication Nos. US 2010/136030, US 2014/377284, US 2015/190506, and US 2015/132288 (including clones 20E5 and 12H3); and U.S. Pat. Nos. 7,504,101, 7,550,140, 7,622,444, 7,696,175, 7,960,515, 7,961,515, 8,133,983, 9,006,399, and 9,163,085, the disclosure of each of which is incorporated herein by reference in its entirety.

In an embodiment, the OX40 agonist is an OX40 agonistic fusion protein as depicted in Structure 1-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A given in FIG. 18 are found in Table 9. The Fc domain preferably comprises a complete constant domain (amino acids 17-230 of SEQ ID NO:62) the complete hinge domain (amino acids 1-16 of SEQ ID NO:62) or a portion of the hinge domain (e.g., amino acids 4-16 of SEQ ID NO:62). Preferred linkers for connecting a C-terminal Fc-antibody may be selected from the embodiments given in SEQ ID NO:63 to SEQ ID NO:72, including linkers suitable for fusion of additional polypeptides. Likewise, amino acid sequences for the polypeptide domains of structure I-B given in FIG. 18 are found in Table 10. If an Fc antibody fragment is fused to the N-terminus of an TNRFSF fusion protein as in structure I-B, the sequence of the Fc module is preferably that shown in SEQ ID NO:73, and the linker sequences are preferably selected from those embodiments set forth in SED ID NO:74 to SEQ ID NO:76.

In an embodiment, an OX40 agonist fusion protein according to structures I-A or I-B comprises one or more OX40 binding domains selected from the group consisting of a variable heavy chain and variable light chain of tavolixizumab, a variable heavy chain and variable light chain of 11D4, a variable heavy chain and variable light chain of 18D8, a variable heavy chain and variable light chain of Hu119-122, a variable heavy chain and variable light chain of Hu106-222, a variable heavy chain and variable light chain selected from the variable heavy chains and variable light chains described in Table 17, any combination of a variable heavy chain and variable light chain of the foregoing, and fragments, derivatives, conjugates, variants, and biosimilars thereof.

In an embodiment, an OX40 agonist fusion protein according to structures I-A or I-B comprises one or more OX40 binding domains comprising an OX40L sequence. In an embodiment, an OX40 agonist fusion protein according to structures I-A or I-B comprises one or more OX40 binding domains comprising a sequence according to SEQ ID NO:133. In an embodiment, an OX40 agonist fusion protein according to structures I-A or I-B comprises one or more OX40 binding domains comprising a soluble OX40L sequence. In an embodiment, a OX40 agonist fusion protein according to structures I-A or I-B comprises one or more OX40 binding domains comprising a sequence according to SEQ ID NO:134. In an embodiment, a OX40 agonist fusion protein according to structures I-A or I-B comprises one or more OX40 binding domains comprising a sequence according to SEQ ID NO:135.

In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:89 and SEQ ID NO:90, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:99 and SEQ ID NO:100, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:109 and SEQ ID NO:110, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:127 and SEQ ID NO:128, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:125 and SEQ ID NO:126, respectively, wherein the V_{H} and V_{L} domains are connected by a linker. In an embodiment, an OX40 agonist fusion protein according to structures I-A or I-B comprises one or more OX40 binding domains that is a scFv domain comprising V_{H} and V_{L} regions that are each at least 95% identical to the V_{H} and V_{L} sequences given in Table 17, wherein the V_{H} and V_{L} domains are connected by a linker.

TABLE 17
Additional polypeptide domains useful as OX40 binding domains in fusion
proteins (e.g., structures I-A and I-B) or as scFv OX40 agonist antibodies.

| Identifier | Sequence (One-Letter Amino Acid Symbols) | |
|---|---|---|
| SEQ ID NO: 133 | MERVQPLEEN VGNAARPRFE RNKLLIVASV IQGLGLLLCF TYICLHFSAL QVSHRYPRIQ | 60 |
| OX40L | SIKVQFTEYK KEKGFILTSQ KEDEIMKVQN NSVIINCDGF YLISLKGYFS QEVNISLEYQ | 120 |
| | KDEEPLFQLK KVRSVNSLMV ASLTYKDKVY LNVTTDNTSL DDFHVNGGEL ILIHQNPGEF | 180 |
| | CVL | 183 |
| SEQ ID NO: 134 | SHRYPRIQSI KVQFTEYKKE KGFILTSQKE DEIMKVQNNS VIINCDGFYL ISLKGYFSQE | 60 |
| OX40L soluble | VNISLHYQKD EEPLFQLKKV RSVNSIMVAS LTYKDKVYLN VTTDNTSLDD FHVNGGELIL | 120 |
| domain | IHQNPGEFCV L | 131 |
| SEQ ID NO: 135 | YPRIQSIKVQ FTEYKKEKGF ILTSQKEDEI MKVQNNSVII NCDGFYLISL KGYFSQEVNI | 60 |
| OX40L soluble | SLHYQKDEEP LFQLKKVRSV NSLMVASLTY KDKVYLNVTT DNTSLDDFHV NGGELILIHQ | 120 |
| domain | NPGEFCVL | 128 |
| (alternative) | | |
| SEQ ID NO: 136 | EVQLVESGGG LVQPGGSLRL SCAASGFTFS NYTMNWVRQA PGKGLEWVSA ISGSGGSTYY | 60 |
| variable heavy | ADSVKGRFTI SRDNSKNTLY LQMNSLRAED TAVYYCAKDR YSQVHYALDY WGQGTLVTVS | 120 |
| chain for 008 | | |
| SEQ ID NO: 137 | DIVMTQSPDS LPVTPGEPAS ISCRSSQSLL HSNGYNYLDW YLQKAGQSPQ LLIYLGSNRA | 60 |
| variable light | SGVPDRFSGS GSGTDETLKI SRVEAEDVGV YYCQQYYNHP TTFGQGTK | 108 |
| chain for 008 | | |
| SEQ ID NO: 138 | EVQLVESGGG VVQPGRSLRL SCAASGFTFS DYTMNWVRQA PGKGLEWVSS ISGGSTYYAD | 60 |
| variable heavy | SRKGRFTISR DNSKNTLYLQ MNNLRAEDTA VYYCARDRYF RQQNAFDYWG QGTLVTVSSA | 120 |
| chain for 011 | | |
| SEQ ID NO: 139 | DIVMTQSPDS LPVTPGEPAS ISCRSSQSLL HSNGYNYLDW YLQKAGQSPQ LLIYLGSNRA | 60 |
| variable light | SGVPDRFSGS GSGTDETLKI SRVEAEDVGV YYCQQYYNHP TTFGQGTK | 108 |
| chain for 011 | | |
| SEQ ID NO: 140 | EVQLVESGGG LVQPRGSLRL SCAASGFTFS SYAMNWVRQA PGKGLEWVAV ISYDGSNKYY | 60 |
| variable heavy | ADSVKGRFTI SRDNSKNTLY LQMNSLRAED TAVYYCAKDR YITLPNALDY WGQGTLVTVS | 120 |
| chain for 021 | | |
| SEQ ID NO: 141 | DIQMTQSPVS LPVTPGEPAS ISCRSSQSLL HSNGYNYLDW YLQKPGQSPQ LLIYLGSNRA | 60 |
| variable light | SGVPDRFSGS GSGTDETLKI SRVEAEDVGV YYCQQYKSNP PTFGQGTK | 108 |
| chain for 021 | | |
| SEQ ID NO: 142 | EVQLVESGGG LVHPGGSLRL SCAGSGFTFS SYAMHWVRQA PGKGLEWVSA IGTGGGTYYA | 60 |
| variable heavy | DSVMGRFTIS RDNSKNTLYL QMNSLRAEDT AVYYCARYDN VMGLYWFDYW GQGTLVTVSS | 120 |
| chain for 023 | | |
| SEQ ID NO: 143 | EIVLTQSPAT LSLSPGERAT LSCRASQSVS SYLAWYQQKP GQAPRLLIYD ASNRATGIPA | 60 |
| variable light | RFSGSGSGTD FTLTISSLEP EDFAVYYCQQ RSNWPPAFGG GTKVEIKR | 108 |
| chain for 023 | | |
| SEQ ID NO: 144 | EVQLQQSGPE LVKPGASVKM SCKASGYTFT SYVMHWVKQK PGQGLEWIGY INPYNDGTKY | 60 |
| heavy chain | NEKFKGKATL TSDKSSSTAY MELSSITSED SAVYYCANYY GSSLSMDYWG QGTSVTVSS | 119 |
| variable region | | |
| SEQ ID NO: 145 | DIQMTQTTSS LSASLGDRVT ISCRASQDIS NYLNWYQQKP DGTVKLLIYY TSRLHSGVPS | 60 |
| light chain | RFSGSGSGTD YSLTISNLEQ EDIATYFCQQ GNTLPWTFGG GTKLEIKR | 108 |
| variable region | | |
| SEQ ID NO: 146 | EVQLQQSGPE LVKPGASVKI SCKTSGYTFK DYTMHWVKQS HGKSLEWIGG IYPNNGGSTY | 60 |
| heavy chain | NQNFKDKATL TVDKSSSTAY MEFRSITSED SAVYYCARMG YHGPHLDFDV WGAGTTVTVS | 120 |
| variable region | P | 121 |
| SEQ ID NO: 147 | DIVMTQSHKF MSTSLGDRVS ITCKASQDVG AAVAWYQQKP GQSPKLLIYW ASTRHTGVPD | 60 |
| light chain | RFTGGGSGTD FTLTISNVQS EDLTDYFCQQ YINYPLEFGG GTKLEIKR | 108 |
| variable region | | |
| SEQ ID NO: 148 | QIQLVQSGPE LKKPGETVKI SCKASGYTFT DYSMHWVKQA PGKGLKWMGW INTETGEPTY | 60 |
| heavy chain | ADDFKGRFAF SLETSASTAY LQINNLKNED TATYFCANPY YDYVSYYAMD YWGHGTSVTV | 120 |
| variable region | SS | 122 |
| of humanized | | |
| antibody | | |
| SEQ ID NO: 149 | QVQLVQSGSE LKKPGASVKV SCKASGYTFT DYSMHWVRQA PGQGLKWMGW INTETGEPTY | 60 |
| heavy chain | ADDFKGRFVF SLDTSVSTAY LQISSIKAED TAVYYCANPY YDYVSYYAMD YWGQGTTVTV | 120 |
| variable region | SS | 122 |
| of humanized | | |
| antibody | | |
| SEQ ID NO: 150 | DIVMTQSHKF MSTSVRDRVS ITCKASQDVS TAVAWYQQKP GQSPKLLIYS ASYLYTGVPD | 60 |
| light chain | RFTGSGSGTD FTFTISSVQA EDLAVYYCQQ HYSTPREFGG GTKLEIK | 107 |
| variable region | | |
| of humanized | | |
| antibody | | |
| SEQ ID NO: 151 | DIVMTQSHKF MSTSVRDRVS ITCKASQDVS TAVAWYQQKP GQSPKLLIYS ASYLYTGVPD | 60 |
| light chain | RFTGSGSGTD FTFTISSVQA EDLAVYYCQQ HYSTPREFGG GTKLEIK | 107 |
| variable region | | |
| of humanized | | |
| antibody | | |
| SEQ ID NO: 152 | EVQLVESGGG LVQPGESLKL SCESNEYEFP SHDMSWVRKT PEKRLELVAA INSEGGSTYY | 60 |
| heavy chain | PDTMERRFII SRDNTKKTLY LQMSSLRSED TALYYCARHY DDYYAWFAYW GQGTLVTVSA | 120 |
| variable region | | |
| of humanized | | |
| antibody | | |
| SEQ ID NO: 153 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY LQMNSIRAED TAVYYCARHY DDYYAWFAYW GQGTMVTVSS | 120 |
| variable region | | |
| of humanized | | |
| antibody | | |
| SEQ ID NO: 154 | DIVLTQSPAS LAVSLGQRAT ISCRASKSVS TSGYSYMHWY QQKPGQPPKL LIYLASNLES | 60 |
| light chain | GVPARFSGSG SGTDFTLNIH PVEEEDAATY YCQHSRELPL TEGAGTKLEL K | 111 |
| variable region | | |
| of humanized | | |
| antibody | | |
| SEQ ID NO: 155 | EIVLTQSPAT LSLSPGERAT LSCRASKSVS TSGYSYMHWY QQKPGQAPRL LIYLASNLES | 60 |
| light chain | | |
| variable region | | |
| of humanized | GVPARFSGSG SGTDFTLTIS SLEPEDFAVY YCQHSRELPL TEGGGTKVEI K | 111 |
| antibody | | |
| SEQ ID NO: 156 | MYLGLNYVFI VFLLNGVQSE VKLEESGGGL VQPGGSMKLS CAASGFTFSD AWMDWVRQSP | 60 |
| heavy chain | EKGLEWVAEI RSKANNHATY YAESVNGRFT ISRDDSKSSV YLQMNSLRAE DTGIYYCTWG | 120 |
| variable region | EVFYFDYWGQ GTTLTVSS | 138 |
| SEQ ID NO: 157 | MRPSIQFLGL LLFWLHGAQC DIQMTQSPSS LSASLGGKVT ITCKSSQDIN KYIAWYQEKP | 60 |
| light chain | GKGPRLLIHY TSTLQPGIPS RFSGSGSGRD YSFSISNLEP EDIATYYCLQ YDNLLTFGAG | 120 |
| variable region | TKLELK | 126 |

In an embodiment, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, and wherein the additional domain is a Fab or Fc fragment domain. In an embodiment, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, wherein the additional domain is a Fab or Fc fragment domain wherein each of the soluble OX40 binding domains lacks a stalk region (which contributes to trimerization and provides a certain distance to the cell membrane, but is not part of the OX40 binding domain) and the first and the second peptide linkers independently have a length of 3-8 amino acids.

In an embodiment, the OX40 agonist is an OX40 agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an OX40 binding domain.

In some embodiments, the OX40 agonist is MEDI6383. MEDI6383 is an OX40 agonistic fusion protein and can be prepared as described in U.S. Pat. No. 6,312,700, the disclosure of which is incorporated by reference herein.

In an embodiment, the OX40 agonist is an OX40 agonistic scFv antibody comprising any of the foregoing V_{H} domains linked to any of the foregoing V_{L} domains.

In an embodiment, the OX40 agonist is Creative Biolabs OX40 agonist monoclonal antibody MOM-18455, commercially available from Creative Biolabs, Inc., Shirley, NY, USA.

In an embodiment, the OX40 agonist is OX40 agonistic antibody clone Ber-ACT35 commercially available from BioLegend, Inc., San Diego, CA, USA.

### 4. AKT Inhibitors and DNA Hypomethylation Agents

In an embodiment, the cell culture medium of the first expansion and/or the rapid second expansion of either a Gen 2 or Gen 3 process, or other processes described herein, comprises an AKT inhibitor. The use of an AKT inhibitor in TIL, MIL, and PBL expansion processes is described in International Patent Publication No. WO 2020/096927 A1, the disclosures of which are incorporated by reference herein. The AKT inhibitors disclosed herein may be used with the CCRs and chemokine receptors disclosed herein, in connection with the processes disclosed herein, or may be used alone with the processes disclosed herein (such as the Gen 2 or Gen 3 process) without use of CCR or chemokine receptor modifications.

Suitable AKT inhibitors include AKT1, AKT2, and/or AKT3 inhibitors. In some embodiments, the AKT inhibitor is afuresertib, or pharmaceutically acceptable salts, solvates, hydrates, cocrystals, or prodrugs thereof, and combinations thereof. In some embodiments, the AKT inhibitor is ipatasertib, or pharmaceutically acceptable salts, solvates, hydrates, cocrystals, or prodrugs thereof, and combinations thereof. In some embodiments, the AKT inhibitor is selected from the group consisting of afuresertib, uprosertib, ipatasertib, borussertib, capivasertib, miransertib, oridonin, vevorisertib, AT7867, AT13148, BAY1125976, GSK-690693, MK-2206, LY294002, PF-04691502 or pharmaceutically acceptable salts, solvates, hydrates, cocrystals, or prodrugs thereof, and combinations thereof. In some embodiments, the AKT inhibitor is added to the media during the pre-REP stage of a Gen 2 process, such as immediately after the fragmented or digested tumor addition to culture. In some embodiments, the AKT inhibitor is added to the media during the REP stage of a Gen 2 process. In some embodiments, the AKT inhibitor is added to the media during the priming stage of a Gen 3 process. In some embodiments, the AKT inhibitor is added to the media during the REP stage of a Gen 3 process. In some embodiments, the AKT inhibitor is added to the media during the REP stage of a TIL, MIL, or PBL manufacturing process. In some embodiments, the AKT inhibitor is added to the media during TIL expansion. In some embodiments, the AKT inhibitor is added to the media during a TIL expansion process that includes a genetic modification step described herein. In some embodiments, the AKT inhibitor is added to the media during a TIL expansion process that includes a transduction step for a CCR or chemokine receptor. In some embodiments, the AKT inhibitor is an allosteric AKT inhibitor. In some embodiments, the AKT inhibitor is a covalent AKT inhibitor.

In some embodiments, use of an AKT inhibitor during TIL expansion results in TILs with differentiated CD39⁻CD69⁻ cells. In some embodiments, use of an AKT inhibitor during TIL expansion results in TILs with at least 10%, 20%, 30%, 40%, 50%, 60%, 70%, 80%, 90% or 100% (i.e., doubling) of the amount of CD39⁻CD69⁻ cells relative to TILs prepared without using an AKT inhibitor, for example, using the Gen 2 or Gen 3 process, modified for expression of a CCR or chemokine receptor. In some embodiments, use of an AKT inhibitor during TIL expansion results in TILs with at least 10%, 20%, 30%, 40%, 50%, 60%, 70%, 80% 90% or 100% (i.e., doubling) of IFNγ⁺TNFα⁺ CD8⁺ T cells.

In an embodiment, the invention includes a therapeutic TIL composition comprising TILs comprising a CCR or chemokine receptor, wherein the TILs are further optionally modified to either stably or transiently reduce expression of a protein (such as PD-1) by knockout or knockdown of a gene (such as PDCD1), and/or wherein the TILs are prepared with an AKT inhibitor and exhibit an increase in the amount of CD39⁻CD69⁻ cells relative to TILs prepared without using an AKT inhibitor.

In an embodiment, the cell culture medium of the first expansion and/or the rapid second expansion of either a Gen 2 or Gen 3 process, or other processes described herein, comprises decitabine, or a salt, cocrystal, solvate, or hydrate thereof, alone or in addition to an AKT inhibitor, and alone or in conjunction with the CCRs and chemokine receptors disclosed herein.

### C. Optional Cell Viability Analyses

Optionally, a cell viability assay can be performed after the priming first expansion (sometimes referred to as the initial bulk expansion), using standard assays known in the art. Thus, in certain embodiments, the method comprises performing a cell viability assay subsequent to the priming first expansion. For example, a trypan blue exclusion assay can be done on a sample of the bulk TILs, which selectively labels dead cells and allows a viability assessment. Other assays for use in testing viability can include but are not limited to the Alamar blue assay and the MTT assay.

### 1. Cell Counts, Viability, Flow Cytometry

In some embodiments, cell counts and/or viability are measured. The expression of markers such as but not limited CD3, CD4, CD8, and CD56, as well as any other disclosed or described herein, can be measured by flow cytometry with antibodies, for example but not limited to those commercially available from BD Biosciences (San Jose, CA) using a FACSCanto™ flow cytometer (BD Biosciences). The cells can be counted manually using a disposable c-chip hemocytometer (VWR, Batavia, IL) and viability can be assessed using any method known in the art, including but not limited to trypan blue staining. The cell viability can also be assayed based on U.S. Patent Application Publication No. 2018/0282694, incorporated by reference herein in its entirety. Cell viability can also be assayed based on U.S. Patent Application Publication No. 2018/0280436 or International Patent Application Publication No. WO/2018/081473, both of which are incorporate herein in their entireties for all purposes.

In some cases, the bulk TIL population can be cryopreserved immediately, using the protocols discussed below. Alternatively, the bulk TIL population can be subjected to REP and then cryopreserved as discussed below. Similarly, in the case where genetically modified TILs will be used in therapy, the bulk or REP TIL populations can be subjected to genetic modifications for suitable treatments.

### 2. Cell Cultures

In an embodiment, a method for expanding TILs, including those discussed above as well as exemplified in FIGS. 1 and 8, in particular, e.g., FIG. 8B and/or FIG. 8C, may include using about 5,000 mL to about 25,000 mL of cell medium, about 5,000 mL to about 10,000 mL of cell medium, or about 5,800 mL to about 8,700 mL of cell medium. In some embodiments, the media is a serum free medium. In some embodiments, the media in the priming first expansion is serum free. In some embodiments, the media in the second expansion is serum free. In some embodiments, the media in the priming first expansion and the second expansion (also referred to as rapid second expansion) are both serum free. In an embodiment, expanding the number of TILs uses no more than one type of cell culture medium. Any suitable cell culture medium may be used, e.g., AIM-V cell medium (L-glutamine, 50 μM streptomycin sulfate, and 10 μM gentamicin sulfate) cell culture medium (Invitrogen, Carlsbad CA). In this regard, the inventive methods advantageously reduce the amount of medium and the number of types of medium required to expand the number of TIL. In an embodiment, expanding the number of TIL may comprise feeding the cells no more frequently than every third or fourth day. Expanding the number of cells in a gas permeable container simplifies the procedures necessary to expand the number of cells by reducing the feeding frequency necessary to expand the cells.

In an embodiment, the cell culture medium in the first and/or second gas permeable container is unfiltered. The use of unfiltered cell medium may simplify the procedures necessary to expand the number of cells. In an embodiment, the cell medium in the first and/or second gas permeable container lacks beta-mercaptoethanol (BME).

In an embodiment, the duration of the method comprising obtaining a tumor tissue sample from the mammal; culturing the tumor tissue sample in a first gas permeable container containing cell medium including IL-2, 1× antigen-presenting feeder cells, and OKT-3 for a duration of about 1 to 8 days, e.g., about 7 days as a priming first expansion, or about 8 days as a priming first expansion; transferring the TILs to a second gas permeable container and expanding the number of TILs in the second gas permeable container containing cell medium including IL-2, 2× antigen-presenting feeder cells, and OKT-3 for a duration of about 7 to 9 days, e.g., about 7 days, about 8 days, or about 9 days.

In an embodiment, the duration of the method comprising obtaining a tumor tissue sample from the mammal; culturing the tumor tissue sample in a first gas permeable container containing cell medium including IL-2, 1× antigen-presenting feeder cells, and OKT-3 for a duration of about 1 to 7 days, e.g., about 7 days as a priming first expansion; transferring the TILs to a second gas permeable container and expanding the number of TILs in the second gas permeable container containing cell medium including IL-2, 2× antigen-presenting feeder cells, and OKT-3 for a duration of about 7 to 14 days, or about 7 to 9 days, e.g., about 7 days, about 8 days, or about 9 days, about 10 days, or about 11 days.

In an embodiment, the duration of the method comprising obtaining a tumor tissue sample from the mammal; culturing the tumor tissue sample in a first gas permeable container containing cell medium including IL-2, 1× antigen-presenting feeder cells, and OKT-3 for a duration of about 1 to 7 days, e.g., about 7 days, as a priming first expansion; transferring the TILs to a second gas permeable container and expanding the number of TILs in the second gas permeable container containing cell medium including IL-2, 2× antigen-presenting feeder cells, and OKT-3 for a duration of about 7 to 11 days, e.g., about 7 days, about 8 days, about 9 days, about 10, or about 11 days.

In an embodiment, TILs are expanded in gas-permeable containers. Gas-permeable containers have been used to expand TILs using PBMCs using methods, compositions, and devices known in the art, including those described in U.S. Patent Application Publication No. 2005/0106717 A1, the disclosures of which are incorporated herein by reference. In an embodiment, TILs are expanded in gas-permeable bags. In an embodiment, TILs are expanded using a cell expansion system that expands TILs in gas permeable bags, such as the Xuri Cell Expansion System W25 (GE Healthcare). In an embodiment, TILs are expanded using a cell expansion system that expands TILs in gas permeable bags, such as the WAVE Bioreactor System, also known as the Xuri Cell Expansion System W5 (GE Healthcare). In an embodiment, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L.

In an embodiment, TILs can be expanded in G-Rex flasks (commercially available from Wilson Wolf Manufacturing). Such embodiments allow for cell populations to expand from about 5×10⁵ cells/cm² to between 10×10⁶ and 30×10⁶ cells/cm². In an embodiment this is without feeding. In an embodiment, this is without feeding so long as medium resides at a height of about 10 cm in the G-Rex flask. In an embodiment this is without feeding but with the addition of one or more cytokines. In an embodiment, the cytokine can be added as a bolus without any need to mix the cytokine with the medium. Such containers, devices, and methods are known in the art and have been used to expand TILs, and include those described in U.S. Patent Application Publication No. US 2014/0377739A1, International Publication No. WO 2014/210036 A1, U.S. Patent Application Publication No. us 2013/0115617 A1, International Publication No. WO 2013/188427 A1, U.S. Patent Application Publication No. US 2011/0136228 A1, U.S. Pat. No. 8,809,050 B2, International publication No. WO 2011/072088 A2, U.S. Patent Application Publication No. US 2016/0208216 A1, U.S. Patent Application Publication No. US 2012/0244133 A1, International Publication No. WO 2012/129201 A1, U.S. Patent Application Publication No. US 2013/0102075 A1, U.S. Pat. No. 8,956,860 B2, International Publication No. WO 2013/173835 A1, U.S. Patent Application Publication No. US 2015/0175966 A1, the disclosures of which are incorporated herein by reference. Such processes are also described in Jin et al., J. Immunotherapy, 2012, 35:283-292.

### D. Optional Knockdown or Knockout of Genes in TILs

In some embodiments, the expanded TILs of the present invention are further manipulated before, during, or after an expansion step, including during closed, sterile manufacturing processes, each as provided herein, in order to alter protein expression in a transient manner. In some embodiments, the transiently altered protein expression is due to transient gene editing. In some embodiments, the expanded TILs of the present invention are treated with transcription factors (TFs) and/or other molecules capable of transiently altering protein expression in the TILs. In some embodiments, the TFs and/or other molecules that are capable of transiently altering protein expression provide for altered expression of tumor antigens and/or an alteration in the number of tumor antigen-specific T cells in a population of TILs.

In certain embodiments, the method comprises genetically editing a population of TILs. In certain embodiments, the method comprises genetically editing the first population of TILs, the second population of TILs and/or the third population of TILs.

In some embodiments, the present invention includes genetic editing through nucleotide insertion, such as through ribonucleic acid (RNA) insertion, including insertion of messenger RNA (mRNA) or small (or short) interfering RNA (siRNA), into a population of TILs for promotion of the expression of one or more proteins or inhibition of the expression of one or more proteins, as well as simultaneous combinations of both promotion of one set of proteins with inhibition of another set of proteins.

In some embodiments, the expanded TILs of the present invention undergo transient alteration of protein expression. In some embodiments, the transient alteration of protein expression occurs in the bulk TIL population prior to first expansion. In some embodiments, the transient alteration of protein expression occurs after the first expansion. In some embodiments, the transient alteration of protein expression occurs in the bulk TIL population prior to second expansion. In some embodiments, the transient alteration of protein expression occurs after the second expansion.

In an embodiment, a method of transiently altering protein expression in a population of TILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. In an embodiment, a method of transiently altering protein expression in population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of transiently altering protein expression in a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of transiently altering protein expression in a population of TILs includes the step of transfection using methods described in U.S. Pat. Nos. 5,766,902; 6,025,337; 6,410,517; 6,475,994; and 7,189,705; the disclosures of each of which are incorporated by reference herein.

In some embodiments, the TILs of the present invention, including TILs modified to express CCRs, are further modified to transiently or permanently suppress the expression of one or more genes using the methods described in International Patent Application Nos. WO 2019/136456 A1 or WO 2019/210131 A1, the disclosures of each of which are incorporated by reference herein, including methods described therein to genetically edit TILs to knockout specific target genes such as the genes that code for PD-1 and CTLA-4.

In some embodiments, transient alteration of protein expression results in an increase in stem memory T cells (TSCMs). TSCMs are early progenitors of antigen-experienced central memory T cells. TSCMs generally display the long-term survival, self-renewal, and multipotency abilities that define stem cells, and are generally desirable for the generation of effective TIL products. TSCM have shown enhanced anti-tumor activity compared with other T cell subsets in mouse models of adoptive cell transfer. In some embodiments, transient alteration of protein expression results in a TIL population with a composition comprising a high proportion of TSCM. In some embodiments, transient alteration of protein expression results in an at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% increase in TSCM percentage. In some embodiments, transient alteration of protein expression results in an at least a 1-fold, 2-fold, 3-fold, 4-fold, 5-fold, or 10-fold increase in TSCMs in the TIL population. In some embodiments, transient alteration of protein expression results in a TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs. In some embodiments, transient alteration of protein expression results in a therapeutic TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs.

In some embodiments, transient alteration of protein expression results in rejuvenation of antigen-experienced T-cells. In some embodiments, rejuvenation includes, for example, increased proliferation, increased T-cell activation, and/or increased antigen recognition.

In some embodiments, transient alteration of protein expression alters the expression in a large fraction of the T-cells in order to preserve the tumor-derived TCR repertoire. In some embodiments, transient alteration of protein expression does not alter the tumor-derived TCR repertoire. In some embodiments, transient alteration of protein expression maintains the tumor-derived TCR repertoire.

In some embodiments, transient alteration of protein results in altered expression of a particular gene. In some embodiments, the transient alteration of protein expression targets a gene including but not limited to PD-1 (also referred to as PDCD1 or CC279), TGFBR2, CCR4/5, CBL-B (also known as CBLB and Cbl-b), CISH, CCRs (chimeric co-stimulatory receptors), IL-2, IL-12, IL-15, IL-21, NOTCH 1/2 ICD, TIM-3, LAG-3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, thymocyte selection associated high mobility group (HMG) box (TOX), ankyrin repeat domain 11 (ANKRD11), BCL6 co-repressor (BCOR) and/or cAMP protein kinase A (PKA). In some embodiments, the transient alteration of protein expression targets a gene selected from the group consisting of PD-1, TGFBR2, CCR4/5, CBL-B, CISH, CCRs (chimeric co-stimulatory receptors), IL-2, IL-12, IL-15, IL-21, NOTCH 1/2 ICD, TIM-3, LAG-3, TIGIT, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, thymocyte selection associated high mobility group (HMG) box (TOX), ankyrin repeat domain 11 (ANKRD11), BCL6 co-repressor (BCOR) and/or cAMP protein kinase A (PKA). In some embodiments, the transient alteration of protein expression targets PD-1. In some embodiments, the transient alteration of protein expression targets TGFBR2. In some embodiments, the transient alteration of protein expression targets CCR4/5. In some embodiments, the transient alteration of protein expression targets CBL-B. In some embodiments, the transient alteration of protein expression targets CISH. In some embodiments, the transient alteration of protein expression targets CCRs (chimeric co-stimulatory receptors). In some embodiments, the transient alteration of protein expression targets IL-2. In some embodiments, the transient alteration of protein expression targets IL-12. In some embodiments, the transient alteration of protein expression targets IL-15. In some embodiments, the transient alteration of protein expression targets IL-21. In some embodiments, the transient alteration of protein expression targets NOTCH 1/2 ICD. In some embodiments, the transient alteration of protein expression targets TIM-3. In some embodiments, the transient alteration of protein expression targets LAG-3. In some embodiments, the transient alteration of protein expression targets TIGIT. In some embodiments, the transient alteration of protein expression targets TGFβ. In some embodiments, the transient alteration of protein expression targets CCR1. In some embodiments, the transient alteration of protein expression targets CCR2. In some embodiments, the transient alteration of protein expression targets CCR4. In some embodiments, the transient alteration of protein expression targets CCR5. In some embodiments, the transient alteration of protein expression targets CXCR1. In some embodiments, the transient alteration of protein expression targets CXCR2. In some embodiments, the transient alteration of protein expression targets CSCR3. In some embodiments, the transient alteration of protein expression targets CCL2 (MCP-1). In some embodiments, the transient alteration of protein expression targets CCL3 (MIP-1α). In some embodiments, the transient alteration of protein expression targets CCL4 (MIP1-β). In some embodiments, the transient alteration of protein expression targets CCL5 (RANTES). In some embodiments, the transient alteration of protein expression targets CXCL1. In some embodiments, the transient alteration of protein expression targets CXCL8. In some embodiments, the transient alteration of protein expression targets CCL22. In some embodiments, the transient alteration of protein expression targets CCL17. In some embodiments, the transient alteration of protein expression targets VHL. In some embodiments, the transient alteration of protein expression targets CD44. In some embodiments, the transient alteration of protein expression targets PIK3CD. In some embodiments, the transient alteration of protein expression targets SOCS1. In some embodiments, the transient alteration of protein expression targets thymocyte selection associated high mobility group (HMG) box (TOX). In some embodiments, the transient alteration of protein expression targets ankyrin repeat domain 11 (ANKRD11). In some embodiments, the transient alteration of protein expression targets BCL6 co-repressor (BCOR). In some embodiments, the transient alteration of protein expression targets cAMP protein kinase A (PKA).

In some embodiments, the transient alteration of protein expression results in increased and/or overexpression of a chemokine receptor. In some embodiments, the chemokine receptor that is overexpressed by transient protein expression includes a receptor with a ligand that includes but is not limited to CCL2 (MCP-1), CCL3 (MIP-1α), CCL4 (MIP1-β), CCL5 (RANTES), CXCL1, CXCL8, CCL22, and/or CCL17.

In some embodiments, the transient alteration of protein expression results in a decrease and/or reduced expression of PD-1, CTLA-4, TIM-3, LAG-3, TIGIT, TGFβR2, and/or TGFβ (including resulting in, for example, TGFβ pathway blockade). In some embodiments, the transient alteration of protein expression results in a decrease and/or reduced expression of CBL-B. In some embodiments, the transient alteration of protein expression results in a decrease and/or reduced expression of CISH.

In some embodiments, the transient alteration of protein expression results in increased and/or overexpression of chemokine receptors in order to, for example, improve TIL trafficking or movement to the tumor site. In some embodiments, the transient alteration of protein expression results in increased and/or overexpression of a CCR (chimeric co-stimulatory receptor). In some embodiments, the transient alteration of protein expression results in increased and/or overexpression of a chemokine receptor selected from the group consisting of CCR1, CCR2, CCR4, CCR5, CXCR1, CXCR2, and/or CSCR3.

In some embodiments, the transient alteration of protein expression results in increased and/or overexpression of an interleukin, including a membrane bound interleukin. In some embodiments, the transient alteration of protein expression results in increased and/or overexpression of an interleukin selected from the group consisting of IL-2, IL-12, IL-15, and/or IL-21. For example, in some embodiments, electroporation of a membrane-bound IL-2, IL-12, IL-15, and/or IL-21 (mbIL-2, mbIL-12, mbIL-15, and/or mbIL-21, and single-chain variants such as single-chain mbIL-12) may be included in the TILs of the present invention, alone or in combination with the CCRs and chemokine receptors described herein, and alone or in combination with the knockdown or knockout of the genes described herein. Compositions and methods relating to the foregoing are described herein and in Zhang, et al., J. Immunother. Cancer 2020, 8, e000210, International Patent Publication No. WO 2020/123716 A1, and U.S Patent Application Publication No. US 2017/0291934 A1, the disclosures of which are incorporated by reference herein.

In some embodiments, the transient alteration of protein expression results in increased and/or overexpression of NOTCH 1/2 ICD. In some embodiments, the transient alteration of protein expression results in increased and/or overexpression of VHL. In some embodiments, the transient alteration of protein expression results in increased and/or overexpression of CD44. In some embodiments, the transient alteration of protein expression results in increased and/or overexpression of PIK3CD. In some embodiments, the transient alteration of protein expression results in increased and/or overexpression of SOCS1. In some embodiments, the transient alteration of protein expression results in increased and/or overexpression of CD40 ligand (CD40L). In some embodiments, the transient alteration of protein expression results in increased and/or overexpression of cAMP protein kinase A (PKA). In some embodiments, the transient alteration of protein expression results in decreased and/or reduced expression of cAMP protein kinase A (PKA).

In some embodiments, the transient alteration of protein expression results in decreased and/or reduced expression of a molecule selected from the group consisting of PD-1, LAG-3, TIM-3, CTLA-4, TIGIT, CISH, TGFβR2, PKA, CBL-B, BAFF (BR3), and combinations thereof. In some embodiments, the transient alteration of protein expression results in decreased and/or reduced expression of two molecules selected from the group consisting of PD-1, LAG-3, TIM-3, CTLA-4, TIGIT, CISH, TGFβR2, PKA, CBL-B, BAFF (BR3), and combinations thereof. In some embodiments, the transient alteration of protein expression results in decreased and/or reduced expression of PD-1 and one molecule selected from the group consisting of LAG-3, TIM-3, CTLA-4, TIGIT, CISH, TGFβR2, PKA, CBL-B, BAFF (BR3), and combinations thereof. In some embodiments, the transient alteration of protein expression results in decreased and/or reduced expression of PD-1, LAG-3, CISH, CBL-B, TIM-3, and combinations thereof. In some embodiments, the transient alteration of protein expression results in decreased and/or reduced expression of PD-1 and one of LAG-3, CISH, CBL-B, TIM-3, and combinations thereof. In some embodiments, the transient alteration of protein expression results in decreased and/or reduced expression of PD-1 and LAG-3. In some embodiments, the transient alteration of protein expression results in decreased and/or reduced expression of PD-1 and CISH. In some embodiments, the transient alteration of protein expression results in decreased and/or reduced expression of PD-1 and CBL-B. In some embodiments, the transient alteration of protein expression results in decreased and/or reduced expression of LAG-3 and CISH. In some embodiments, the transient alteration of protein expression results in decreased and/or reduced expression of LAG-3 and CBL-B. In some embodiments, the transient alteration of protein expression results in decreased and/or reduced expression of CISH and CBL-B. In some embodiments, the transient alteration of protein expression results in decreased and/or reduced expression of TIM-3 and PD-1. In some embodiments, the transient alteration of protein expression results in decreased and/or reduced expression of TIM-3 and LAG-3. In some embodiments, the transient alteration of protein expression results in decreased and/or reduced expression of TIM-3 and CISH. In some embodiments, the transient alteration of protein expression results in decreased and/or reduced expression of TIM-3 and CBL-B.

In some embodiments, an adhesion molecule selected from the group consisting of CCR2, CCR4, CCR5, CXCR2, CXCR3, CX3CR1, and combinations thereof, is inserted by a gammaretroviral or lentiviral method into the first population of TILs, second population of TILs, or harvested population of TILs (e.g., the expression of the adhesion molecule is increased).

In some embodiments, the transient alteration of protein expression results in decreased and/or reduced expression of a molecule selected from the group consisting of PD-1, LAG-3, TIM-3, CTLA-4, TIGIT, CISH, TGFβR2, PKA, CBL-B, BAFF (BR3), and combinations thereof, and increased and/or enhanced expression of CCR2, CCR4, CCR5, CXCR2, CXCR3, CX3CR1, and combinations thereof. In some embodiments, the transient alteration of protein expression results in decreased and/or reduced expression of a molecule selected from the group consisting of PD-1, LAG-3, TIM-3, CISH, CBL-B, and combinations thereof, and increased and/or enhanced expression of CCR2, CCR4, CCR5, CXCR2, CXCR3, CX3CR1, and combinations thereof.

In some embodiments, the TILs are further genetically modified for stable or transient alteration of protein expression targets a gene including, but not limited to, CD38, HPK1, YAP1, PTPN22, CBL-B, PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, CD40L, and/or c-Jun. In some embodiments, the TILs are further genetically modified for alteration of protein expression targets a gene selected from the group consisting of CD38, HPK1, YAP1, PTPN22, CBL-B, PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, CD40L, c-Jun, and combinations thereof.

In some embodiments, the transient alteration of protein expression targets CD38. In some embodiments, the transient alteration of protein expression targets HPK1. In some embodiments, the transient alteration of protein expression targets YAP1. In some embodiments, the transient alteration of protein expression targets PTPN22. In some embodiments, the transient alteration of protein expression targets CBL-B. In some embodiments, the transient alteration of protein expression targets PGC1alpha. In some embodiments, the transient alteration of protein expression targets NT-PGC1alpha. In some embodiments, the transient alteration of protein expression targets CXCR1. In some embodiments, the transient alteration of protein expression targets CXCR2. In some embodiments, the transient alteration of protein expression targets CXCR3. In some embodiments, the transient alteration of protein expression targets CXCR4. In some embodiments, the transient alteration of protein expression targets CXCR5. In some embodiments, the transient alteration of protein expression targets CXCR6. In some embodiments, the transient alteration of protein expression targets CX3CR1. In some embodiments, the transient alteration of protein expression targets CCR1. In some embodiments, the transient alteration of protein expression targets CCR2. In some embodiments, the transient alteration of protein expression targets CCR4. In some embodiments, the transient alteration of protein expression targets CCR5. In some embodiments, the transient alteration of protein expression targets CCR6. In some embodiments, the transient alteration of protein expression targets CCR7. In some embodiments, the transient alteration of protein expression targets CCR8. In some embodiments, the transient alteration of protein expression targets CCR9. In some embodiments, the transient alteration of protein expression targets CCR10. In some embodiments, the transient alteration of protein expression targets BATF. In some embodiments, the transient alteration of protein expression targets c-Jun. In some embodiments, the transient alteration of protein expression targets CD40L.

In some embodiments, the transient alteration of protein expression results in a decrease and/or reduced expression of a molecule or more of selected from the group consisting of CD38, HPK1, YAP1, PTPN22, CBL-B, and combinations thereof. In some embodiments, the transient alteration of protein expression results in a decrease and/or reduced expression of two molecules or more of selected from the group consisting of CD38, HPK1, YAP1, PTPN22, CBL-B, and combinations thereof. In some embodiments, the transient alteration of protein expression results in a decrease and/or reduced expression of three molecules or more of selected from the group consisting of CD38, HPK1, YAP1, PTPN22, CBL-B, and combinations thereof. In some embodiments, the transient alteration of protein expression results in a decrease and/or reduced expression of four molecules or more of selected from the group consisting of CD38, HPK1, YAP1, PTPN22, CBL-B, and combinations thereof. In some embodiments, the transient alteration of protein expression results in a decrease and/or reduced expression of CD38, HPK1 and/or YAP1. In some embodiments, the transient alteration of protein expression results in a decrease and/or reduced expression of CD38 and decrease and/or reduced expression of PTPN22, CBL-B, and combinations thereof. In some embodiments, the transient alteration of protein expression results in a decrease and/or reduced expression of HPK1 and decrease and/or reduced expression of PTPN22, CBL-B. and combinations thereof. In some embodiments, the transient alteration of protein expression results in a decrease and/or reduced expression of YAP1 and decrease and/or reduced expression PTPN22, CBL-B, and combinations thereof. In some embodiments, the transient alteration of protein expression results in a decrease and/or reduced expression of PTPN22 and decrease and/or reduced expression of CD38, HPK1, YAP1, and combinations thereof. In some embodiments, the transient alteration of protein expression results in a decrease and/or reduced expression of CBL-B and decrease and/or reduced expression of CD38, HPK1, YAP1, and combinations thereof.

In some embodiments, the transient alteration of protein expression results in increased and/or overexpression of a molecule or more of selected from the group consisting of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in increased and/or overexpression of two molecules or more of selected from the group consisting of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in increased and/or overexpression of three molecules or more of selected from the group consisting of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in increased and/or overexpression of four molecules or more of selected from the group consisting of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in increased and/or overexpression of five molecules or more of selected from the group consisting of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof.

In some embodiments, the transient alteration of protein expression results in decreased and/or reduced expression of a molecule selected from the group consisting of CD38, HPK1, YAP1, PTPN22, CBL-B, and combinations thereof, and increased and/or enhanced expression of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in decreased and/or reduced expression of CD38 and increased and/or enhanced expression of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in decreased and/or reduced expression of HPK1 and increased and/or enhanced expression of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in decreased and/or reduced expression of YAP1 and increased and/or enhanced expression of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in decreased and/or reduced expression of PTPN22 and increased and/or enhanced expression of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof. In some embodiments, the transient alteration of protein expression results in decreased and/or reduced expression of CBL-B and increased and/or enhanced expression of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof.

In some embodiments, the transient alteration of protein expression results in increased and/or enhanced expression of a molecule selected from the group consisting of PGC1alpha, NT-PGC1alpha, CXCR1, CXCR2, CXCR3, CXCR4, CXCR5, CXCR6, CX3CR1, CCR1, CCR2, CCR4, CCR5, CCR6, CCR7, CCR8, CCR9, CCR10, BATF, c-Jun, and combinations thereof and decreased and/or reduced expression of CD38, HPK1, YAP1, PTPN22, CBL-B, and combinations thereof. In some embodiments, the transient alteration of protein expression results in increased and/or enhanced expression of PGC1alpha and decreased and/or reduced expression of CD38, HPK1, YAP1, PTPN22, CBL-B, and combinations thereof. In some embodiments, the transient alteration of protein expression results in increased and/or enhanced expression of NT-PGC1alpha and decreased and/or reduced expression of CD38, HPK1, YAP1, PTPN22, CBL-B, and combinations thereof. In some embodiments, the transient alteration of protein expression results in increased and/or enhanced expression of CXCR1 and decreased and/or reduced expression of CD38, HPK1, YAP1, PTPN22, CBL-B, and combinations thereof. In some embodiments, the transient alteration of protein expression results in increased and/or enhanced expression of CXCR2 and decreased and/or reduced expression of CD38, HPK1, YAP1, PTPN22, CBL-B, and combinations thereof. In some embodiments, the transient alteration of protein expression results in increased and/or enhanced expression of CXCR3 and decreased and/or reduced expression of CD38, HPK1, YAP1, PTPN22, CBL-B, and combinations thereof. In some embodiments, the transient alteration of protein expression results in increased and/or enhanced expression of CXCR4 and decreased and/or reduced expression of CD38, HPK1, YAP1, PTPN22, CBL-B, and combinations thereof. In some embodiments, the transient alteration of protein expression results in increased and/or enhanced expression of CXCR5 and decreased and/or reduced expression of CD38, HPK1, YAP1, PTPN22, CBL-B, and combinations thereof. In some embodiments, the transient alteration of protein expression results in increased and/or enhanced expression of CXCR6 and decreased and/or reduced expression of CD38, HPK1, YAP1, PTPN22, CBL-B, and combinations thereof. In some embodiments, the transient alteration of protein expression results in increased and/or enhanced expression of CX3CR1 and decreased and/or reduced expression of CD38, HPK1, YAP1, PTPN22, CBL-B, and combinations thereof. In some embodiments, the transient alteration of protein expression results in increased and/or enhanced expression of CCR1 and decreased and/or reduced expression of CD38, HPK1, YAP1, PTPN22, CBL-B, and combinations thereof. In some embodiments, the transient alteration of protein expression results in increased and/or enhanced expression of CCR2 and decreased and/or reduced expression of CD38, HPK1, YAP1, PTPN22, CBL-B, and combinations thereof. In some embodiments, the transient alteration of protein expression results in increased and/or enhanced expression of CCR4 and decreased and/or reduced expression of CD38, HPK1, YAP1, PTPN22, CBL-B, and combinations thereof. In some embodiments, the transient alteration of protein expression results in increased and/or enhanced expression of CCR5 and decreased and/or reduced expression of CD38, HPK1, YAP1, PTPN22, CBL-B, and combinations thereof. In some embodiments, the transient alteration of protein expression results in increased and/or enhanced expression of CCR6 and decreased and/or reduced expression of CD38, HPK1, YAP1, PTPN22, CBL-B, and combinations thereof. In some embodiments, the transient alteration of protein expression results in increased and/or enhanced expression of CCR7 and decreased and/or reduced expression of CD38, HPK1, YAP1, PTPN22, CBL-B, and combinations thereof. In some embodiments, the transient alteration of protein expression results in increased and/or enhanced expression of CCR8 and decreased and/or reduced expression of CD38, HPK1, YAP1, PTPN22, CBL-B, and combinations thereof. In some embodiments, the transient alteration of protein expression results in increased and/or enhanced expression of CCR9 and decreased and/or reduced expression of CD38, HPK1, YAP1, PTPN22, CBL-B, and combinations thereof. In some embodiments, the transient alteration of protein expression results in increased and/or enhanced expression of CCR10 and decreased and/or reduced expression of CD38, HPK1, YAP1, PTPN22, CBL-B, and combinations thereof. In some embodiments, the transient alteration of protein expression results in increased and/or enhanced expression of BATF and decreased and/or reduced expression of CD38, HPK1, YAP1, PTPN22, CBL-B, and combinations thereof. In some embodiments, the transient alteration of protein expression results in increased and/or enhanced expression of c-Jun and decreased and/or reduced expression of CD38, HPK1, YAP1, PTPN22, CBL-B, and combinations thereof.

In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

In some embodiments, there is an increase in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is an increase in expression of at least about 65%, about 70%, about 75%, about 80%, about 85%, about 90%, or about 95%. In some embodiments, there is an increase in expression of at least about 75%, about 80%, about 85%, about 90%, or about 95%. In some embodiments, there is an increase in expression of at least about 80%, about 85%, about 90%, or about 95%. In some embodiments, there is an increase in expression of at least about 85%, about 90%, or about 95%. In some embodiments, there is an increase in expression of at least about 80%. In some embodiments, there is an increase in expression of at least about 85%, In some embodiments, there is an increase in expression of at least about 90%. In some embodiments, there is an increase in expression of at least about 95%. In some embodiments, there is an increase in expression of at least about 99%.

In some embodiments, transient alteration of protein expression is induced by treatment of the TILs with transcription factors (TFs) and/or other molecules capable of transiently altering protein expression in the TILs. In some embodiments, the SQZ vector-free microfluidic platform is employed for intracellular delivery of the transcription factors (TFs) and/or other molecules capable of transiently altering protein expression. Such methods demonstrating the ability to deliver proteins, including transcription factors, to a variety of primary human cells, including T cells, have been described in U.S. Patent Application Publication Nos. US 2019/0093073 A1, US 2018/0201889 A1, and US 2019/0017072 A1, the disclosures of each of which are incorporated by reference herein. Such methods can be employed with the present invention in order to expose a population of TILs to transcription factors (TFs) and/or other molecules capable of inducing transient protein expression, wherein said TFs and/or other molecules capable of inducing transient protein expression provide for increased expression of tumor antigens and/or an increase in the number of tumor antigen-specific T cells in the population of TILs, thus resulting in reprogramming of the TIL population and an increase in therapeutic efficacy of the reprogrammed TIL population as compared to a non-reprogrammed TIL population. In some embodiments, the reprogramming results in an increased subpopulation of effector T cells and/or central memory T cells relative to the starting or prior population (i.e., prior to reprogramming) population of TILs, as described herein.

In some embodiments, the transcription factor (TF) includes but is not limited to TCF-1, NOTCH 1/2 TCD, MYB, BATF, CD40L, and/or C-Jun. In some embodiments, the transcription factor (TF) is TCF-1. In some embodiments, the transcription factor (TF) is NOTCH 1/2 ICD. In some embodiments, the transcription factor (TF) is MYB. In some embodiments, the transcription factor (TF) is BATF. In some embodiments, the transcription factor (TF) is c-Jun. In some embodiments, the transcription factor (TF) is CD40L. In some embodiments, the transcription factor (TF) is administered with induced pluripotent stem cell culture (iPSC), such as the commercially available KNOCKOUT Serum Replacement (Gibco/ThermoFisher), to induce additional TIL reprogramming. In some embodiments, the transcription factor (TF) is administered with an iPSC cocktail to induce additional TIL reprogramming. In some embodiments, the transcription factor (TF) is administered without an iPSC cocktail. In some embodiments, reprogramming results in an increase in the percentage of TSCMs. In some embodiments, reprogramming results in an increase in the percentage of TSCMs by about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95% TSCMs.

In some embodiments, a method of transient altering protein expression, as described above, may be combined with a method of genetically modifying a population of TILs, such as the genetic modifications described elsewhere herein to express CCRs, which includes the step of stable incorporation of genes for production of one or more proteins. In certain embodiments, the method comprises a step of genetically modifying a population of TILs. In certain embodiments, the method comprises genetically modifying the first population of TILs, the second population of TILs and/or the third population of TILs. In an embodiment, a method of genetically modifying a population of TILs includes the step of retroviral transduction. In an embodiment, a method of genetically modifying a population of TILs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J Virology 1998, 72, 8463-71, and U.S. Pat. No. 6,627,442, the disclosures of each of which are incorporated by reference herein. In an embodiment, a method of genetically modifying a population of TILs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In an embodiment, a method of genetically modifying a population of TILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, transient alteration of protein expression in TILs is induced by small interfering RNA (siRNA), sometimes known as short interfering RNA or silencing RNA, which is a double stranded RNA molecule, generally 19-25 base pairs in length. siRNA is used in RNA interference (RNAi), where it interferes with expression of specific genes with complementary nucleotide sequences. siRNA may be used to transiently knockdown genes in TILs also modified to CCRs according to the present invention.

In some embodiments, transient alteration of protein expression in TILs is induced by self-delivering RNA interference (sdRNA), which is a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. Small interfering RNA (siRNA), sometimes known as short interfering RNA or silencing RNA, is a double stranded RNA molecule, generally 19-25 base pairs in length. siRNA is used in RNA interference (RNAi), where it interferes with expression of specific genes with complementary nucleotide sequences. sdRNA are covalently and hydrophobically modified RNAi compounds that do not require a delivery vehicle to enter cells. sdRNAs are generally asymmetric chemically modified nucleic acid molecules with minimal double stranded regions. sdRNA molecules typically contain single stranded regions and double stranded regions, and can contain a variety of chemical modifications within both the single stranded and double stranded regions of the molecule. Additionally, the sdRNA molecules can be attached to a hydrophobic conjugate such as a conventional and advanced sterol-type molecule, as described herein. sdRNAs and associated methods for making such sdRNAs have also been described extensively in, for example, U.S. Patent Application Publication Nos. US 2016/0304873 A1, US 2019/0211337 A1, US 2009/0131360 A1, and US 2019/0048341 A1, and U.S. Pat. Nos. 10,633,654 and 10,913,948B2, the disclosures of each of which are incorporated by reference herein. To optimize sdRNA structure, chemistry, targeting position, sequence preferences, and the like, an algorithm has been developed and utilized for sdRNA potency prediction. Based on these analyses, functional sdRNA sequences have been generally defined as having over 70% reduction in expression at 1 pM concentration, with a probability over 40%.

Double stranded DNA (dsRNA) can be generally used to define any molecule comprising a pair of complementary strands of RNA, generally a sense (passenger) and antisense (guide) strands, and may include single-stranded overhang regions. The term dsRNA, contrasted with siRNA, generally refers to a precursor molecule that includes the sequence of an siRNA molecule which is released from the larger dsRNA molecule by the action of cleavage enzyme systems, including Dicer.

In some embodiments, the method comprises transient alteration of protein expression in a population of TILs, including TILs modified to express a CCR, comprising the use of siRNA or sdRNA. Methods of using siRNA and sdRNA have been described in Khvorova and Watts, Nat. Biotechnol. 2017, 35, 238-248; Byrne, et al., J. Ocul. Pharmacol. Ther. 2013, 29, 855-864; and Ligtenberg, et al., Mol. Therapy, 2018, 26, 1482-93, the disclosures of which are incorporated by reference herein. In an embodiment, delivery of siRNA is accomplished using electroporation or cell membrane disruption (such as the squeeze or SQZ method). In an embodiment, delivery of sdRNA to a TIL population is accomplished without use of electroporation, SQZ, or other methods, instead using a 1 to 3 day period in which a TIL population is exposed to sdRNA at a concentration of 1 μM/10,000 TILs in medium. In certain embodiments, the method comprises delivery or siRNA or sdRNA to a TILs population comprising exposing the TILs population to sdRNA a concentration of 1 μM/10,000 TILs in medium for a period of between 1 to 3 days. In an embodiment, delivery of sdRNA to a TIL population is accomplished using a 1 to 3 day period in which a TIL population is exposed to sdRNA at a concentration of 10 μM/10,000 TILs in medium. In an embodiment, delivery of sdRNA to a TIL population is accomplished using a 1 to 3 day period in which a TIL population is exposed to sdRNA at a concentration of 50 μM/10,000 TILs in medium. In an embodiment, delivery of sdRNA to a TIL population is accomplished using a 1 to 3 day period in which a TIL population is exposed to sdRNA at a concentration of between 0.1 μM/10,000 TILs and 50 μM/10,000 TILs in medium. In an embodiment, delivery of sdRNA to a TIL population is accomplished using a 1 to 3 day period in which a TIL population is exposed to sdRNA at a concentration of between 0.1 μM/10.000 TILs and 50 μM/10,000 TILs in medium, wherein the exposure to sdRNA is performed two, three, four, or five times by addition of fresh sdRNA to the media. Other suitable processes are described, for example, in U.S. Patent Application Publication No. US 2011/0039914 A1, US 2013/0131141 A1, and US 2013/0131142 A1, and U.S. Pat. No. 9,080,171, the disclosures of which are incorporated by reference herein.

In some embodiments, siRNA or sdRNA is inserted into a population of TILs during manufacturing. In some embodiments, the sdRNA encodes RNA that interferes with NOTCH 1/2 ICD, PD-1, CTLA-4 TIM-3, LAG-3, TIGIT, TGFβ, TGFBR2, cAMP protein kinase A (PKA), BAFF BR3, CISH, CBL-B, CD38, HPK1, YAP1, and/or PTPN22 and/or. In some embodiments, the reduction in expression is determined based on a percentage of gene silencing, for example, as assessed by flow cytometry and/or qPCR. In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

The self-deliverable RNAi technology based on the chemical modification of siRNAs can be employed with the methods of the present invention to successfully deliver the sdRNAs to the TILs as described herein. The combination of backbone modifications with asymmetric siRNA structure and a hydrophobic ligand (see, for example, Ligtenberg, et al., Mol. Therapy, 2018, 26, 1482-93 and U.S. Patent Application Publication No. 2016/0304873 A1, the disclosures of which are incorporated by reference herein) allow sdRNAs to penetrate cultured mammalian cells without additional formulations and methods by simple addition to the culture media, capitalizing on the nuclease stability of sdRNAs. This stability allows the support of constant levels of RNAi-mediated reduction of target gene activity simply by maintaining the active concentration of sdRNA in the media. While not being bound by theory, the backbone stabilization of sdRNA provides for extended reduction in gene expression effects which can last for months in non-dividing cells.

In some embodiments, over 95% transfection efficiency of TILs and a reduction in expression of the target by various specific siRNAs or sdRNAs occurs. In some embodiments, siRNAs or sdRNAs containing several unmodified ribose residues were replaced with fully modified sequences to increase potency and/or the longevity of RNAi effect. In some embodiments, a reduction in expression effect is maintained for 12 hours, 24 hours, 36 hours, 48 hours, 5 days, 6 days, 7 days, or 8 days or more. In some embodiments, the reduction in expression effect decreases at 10 days or more post siRNA or sdRNA treatment of the TILs. In some embodiments, more than 70% reduction in expression of the target expression is maintained. In some embodiments, more than 70% reduction in expression of the target expression is maintained TILs. In some embodiments, a reduction in expression in the PD-1/PD-L1 pathway allows for the TILs to exhibit a more potent in vivo effect, which is in some embodiments, due to the avoidance of the suppressive effects of the PD-1/PD-L1 pathway. In some embodiments, a reduction in expression of PD-1 by siRNA or sdRNA results in an increase TIL proliferation.

In some embodiments, the sdRNA sequences used in the invention exhibit a 70% reduction in expression of the target gene. In some embodiments, the sdRNA sequences used in the invention exhibit a 75% reduction in expression of the target gene. In some embodiments, the sdRNA sequences used in the invention exhibit an 80% reduction in expression of the target gene. In some embodiments, the sdRNA sequences used in the invention exhibit an 85% reduction in expression of the target gene. In some embodiments, the sdRNA sequences used in the invention exhibit a 90% reduction in expression of the target gene. In some embodiments, the sdRNA sequences used in the invention exhibit a 95% reduction in expression of the target gene. In some embodiments, the sdRNA sequences used in the invention exhibit a 99% reduction in expression of the target gene. In some embodiments, the sdRNA sequences used in the invention exhibit a reduction in expression of the target gene when delivered at a concentration of about 0.25 μM to about 4 μM. In some embodiments, the sdRNA sequences used in the invention exhibit a reduction in expression of the target gene when delivered at a concentration of about 0.25 μM. In some embodiments, the sdRNA sequences used in the invention exhibit a reduction in expression of the target gene when delivered at a concentration of about 0.5 μM. In some embodiments, the sdRNA sequences used in the invention exhibit a reduction in expression of the target gene when delivered at a concentration of about 0.75 μM. In some embodiments, the sdRNA sequences used in the invention exhibit a reduction in expression of the target gene when delivered at a concentration of about 1.0 μM. In some embodiments, the sdRNA sequences used in the invention exhibit a reduction in expression of the target gene when delivered at a concentration of about 1.25 μM. In some embodiments, the sdRNA sequences used in the invention exhibit a reduction in expression of the target gene when delivered at a concentration of about 1.5 μM. In some embodiments, the sdRNA sequences used in the invention exhibit a reduction in expression of the target gene when delivered at a concentration of about 1.75 μM. In some embodiments, the sdRNA sequences used in the invention exhibit a reduction in expression of the target gene when delivered at a concentration of about 2.0 μM. In some embodiments, the sdRNA sequences used in the invention exhibit a reduction in expression of the target gene when delivered at a concentration of about 2.25 μM. In some embodiments, the sdRNA sequences used in the invention exhibit a reduction in expression of the target gene when delivered at a concentration of about 2.5 μM. In some embodiments, the sdRNA sequences used in the invention exhibit a reduction in expression of the target gene when delivered at a concentration of about 2.75 μM. In some embodiments, the sdRNA sequences used in the invention exhibit a reduction in expression of the target gene when delivered at a concentration of about 3.0 μM. In some embodiments, the sdRNA sequences used in the invention exhibit a reduction in expression of the target gene when delivered at a concentration of about 3.25 μM. In some embodiments, the sdRNA sequences used in the invention exhibit a reduction in expression of the target gene when delivered at a concentration of about 3.5 μM. In some embodiments, the sdRNA sequences used in the invention exhibit a reduction in expression of the target gene when delivered at a concentration of about 3.75 μM. In some embodiments, the sdRNA sequences used in the invention exhibit a reduction in expression of the target gene when delivered at a concentration of about 4.0 μM.

In some embodiments, the siRNA or sdRNA oligonucleotide agents comprise one or more modification to increase stability and/or effectiveness of the therapeutic agent, and to effect efficient delivery of the oligonucleotide to the cells or tissue to be treated. Such modifications can include a 2′-O-methyl modification, a 2′-O-fluro modification, a diphosphorothioate modification, 2′ F modified nucleotide, a 2′-O-methyl modified and/or a 2′deoxy nucleotide. In some embodiments, the oligonucleotide is modified to include one or more hydrophobic modifications including, for example, sterol, cholesterol, vitamin D, naphtyl, isobutyl, benzyl, indol, tryptophane, and/or phenyl. In some embodiments, chemically modified nucleotides are combination of phosphorothioates, 2′-O-methyl, 2′deoxy, hydrophobic modifications and phosphorothioates. In some embodiments, the sugars can be modified and modified sugars can include but are not limited to D-ribose, 2′-O-alkyl (including 2′-O-methyl and 2′-O-ethyl), i.e., 2′-alkoxy, 2′-amino, 2′-S-alkyl, 2′-halo (including 2′-fluoro), T-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described in Augustyns, et al., Nucl. Acids. Res. 1992, 18, 4711, the disclosure of which is incorporated by reference herein.

In some embodiments, the double-stranded siRNA or sdRNA oligonucleotide of the invention is double-stranded over its entire length, i.e., with no overhanging single-stranded sequence at either end of the molecule, i.e., is blunt-ended. In some embodiments, the individual nucleic acid molecules can be of different lengths. In other words, a double-stranded siRNA or sdRNA oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). In some embodiments, when a single nucleic acid molecule is used a portion of the molecule at either end can remain single-stranded.

In some embodiments, a double-stranded siRNA or sdRNA oligonucleotide of the invention contains mismatches and/or loops or bulges, but is double-stranded over at least about 70% of the length of the oligonucleotide. In some embodiments, a double-stranded oligonucleotide of the invention is double-stranded over at least about 80% of the length of the oligonucleotide. In another embodiment, a double-stranded siRNA or sdRNA oligonucleotide of the invention is double-stranded over at least about 90%-95% of the length of the oligonucleotide. In some embodiments, a double-stranded siRNA or sdRNA oligonucleotide of the invention is double-stranded over at least about 96%-98% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

In some embodiments, the siRNA or sdRNA oligonucleotide can be substantially protected from nucleases e.g., by modifying the 3′ or 5′ linkages, as described in U.S. Pat. No. 5,849,902, the disclosure of which is incorporated by reference herein. For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃^{2″}), hydrogen phosphonate, or phosphoramidite). “Blocking groups” can also include “end blocking groups” or “exonuclease blocking groups” which protect the 5′ and 3′ termini of the oligonucleotide, including modified nucleotides and non-nucleotide exonuclease resistant structures.

In some embodiments, at least a portion of the contiguous polynucleotides within the siRNA or sdRNA are linked by a substitute linkage, e.g., a phosphorothioate linkage.

In some embodiments, chemical modification can lead to at least a 1.5, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195,200, 225, 250, 275, 300, 325, 350, 375, 400, 425, 450, 475, or 500 enhancement in cellular uptake of an siRNA or sdRNA. In some embodiments, at least one of the C or U residues includes a hydrophobic modification. In some embodiments, a plurality of Cs and Us contain a hydrophobic modification. In some embodiments, at least 10%, 15%, 20%, 30%, 40%, 50%, 55%, 60% 65%, 70%, 75%, 80%, 85%, 90% or at least 95% of the Cs and Us can contain a hydrophobic modification. In some embodiments, all of the Cs and Us contain a hydrophobic modification.

In some embodiments, the siRNA or sdRNA molecules exhibit enhanced endosomal release of through the incorporation of protonatable amines. In some embodiments, protonatable amines are incorporated in the sense strand (in the part of the molecule which is discarded after RISC loading). In some embodiments, the siRNA or sdRNA compounds of the invention comprise an asymmetric compound comprising a duplex region (required for efficient RISC entry of 10-15 bases long) and single stranded region of 4-12 nucleotides long; with a 13 nucleotide duplex. In some embodiments, a 6 nucleotide single stranded region is employed. In some embodiments, the single stranded region of the siRNA or sdRNA comprises 2-12 phosphorothioate internucleotide linkages (referred to as phosphorothioate modifications). In some embodiments, 6-8 phosphorothioate internucleotide linkages are employed. In some embodiments, the siRNA or sdRNA compounds of the invention also include a unique chemical modification pattern, which provides stability and is compatible with RISC entry. The guide strand, for example, may also be modified by any chemical modification which confirms stability without interfering with RISC entry. In some embodiments, the chemical modification pattern in the guide strand includes the majority of C and U nucleotides being 2′ F modified and the 5′ end being phosphorylated.

In some embodiments, at least 30% of the nucleotides in the siRNA or sdRNA are modified. In some embodiments, at least 30%, 31%, 32%, 33%, 34%, 35%, 36%, 37%, 38%, 39%, 40, 41%, 42%, 43%, 44%, 45%, 46%, 47%, 48%, 49%, 50%, 1%5, 2%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the siRNA or sdRNA are modified. In some embodiments, 100% of the nucleotides in the siRNA or sdRNA are modified.

In some embodiments, the siRNA or sdRNA molecules have minimal double stranded regions. In some embodiments the region of the molecule that is double stranded ranges from 8-15 nucleotides long. In some embodiments, the region of the molecule that is double stranded is 8, 9, 10, 11, 12, 13, 14 or 15 nucleotides long. In some embodiments the double stranded region is 13 nucleotides long. There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. In some embodiments, the single stranded region can be 4, 5, 6, 7, 8, 9, 10, 11 or 12 nucleotides long. In some embodiments, the single stranded region can also be less than 4 or greater than 12 nucleotides long. In certain embodiments, the single stranded region is 6 or 7 nucleotides long.

In some embodiments, the siRNA or sdRNA molecules have increased stability. In some instances, a chemically modified siRNA or sdRNA molecule has a half-life in media that is longer than 1, 2, 3, 4, 5, 6, 7, 8, 9, 10, 11, 12, 13, 14, 15, 16, 17, 18, 19, 20, 21, 22, 23, 24 or more than 24 hours, including any intermediate values. In some embodiments, the siRNA or sd-RNA has a half-life in media that is longer than 12 hours.

In some embodiments, the siRNA or sdRNA is optimized for increased potency and/or reduced toxicity. In some embodiments, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. In some embodiments, reduction in 2′F content of a molecule is predicted to reduce toxicity of the molecule. In some embodiments, the number of phosphorothioate modifications in an RNA molecule can influence the uptake of the molecule into a cell, for example the efficiency of passive uptake of the molecule into a cell. In some embodiments, the siRNA or sdRNA has no 2′F modification and yet are characterized by equal efficacy in cellular uptake and tissue penetration.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contain 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

The self-deliverable RNAi technology provides a method of directly transfecting cells with the RNAi agent (whether siRNA, sdRNA, or other RNAi agents), without the need for additional formulations or techniques. The ability to transfect hard-to-transfect cell lines, high in vivo activity, and simplicity of use, are characteristics of the compositions and methods that present significant functional advantages over traditional siRNA-based techniques, and as such, the sdRNA methods are employed in several embodiments related to the methods of reduction in expression of the target gene in the TILs of the present invention. The sdRNA method allows direct delivery of chemically synthesized compounds to a wide range of primary cells and tissues, both ex-vivo and in vivo. The sdRNAs described in some embodiments of the invention herein are commercially available from Advirna LLC, Worcester, MA, USA.

siRNA and sdRNA may be formed as hydrophobically-modified siRNA-antisense oligonucleotide hybrid structures, and are disclosed, for example in Byrne, et al., J. Ocular Pharmacol. Therapeut., 2013, 29, 855-864, the disclosure of which is incorporated by reference herein.

In some embodiments, the siRNA or sdRNA oligonucleotides can be delivered to the TILs described herein using sterile electroporation. In certain embodiments, the method comprises sterile electroporation of a population of TILs to deliver siRNA or sdRNA oligonucleotides.

In some embodiments, the oligonucleotides can be delivered to the cells in combination with a transmembrane delivery system. In some embodiments, this transmembrane delivery system comprises lipids, viral vectors, and the like. In some embodiments, the oligonucleotide agent is a self-delivery RNAi agent, that does not require any delivery agents. In certain embodiments, the method comprises use of a transmembrane delivery system to deliver siRNA or sdRNA oligonucleotides to a population of TILs.

Oligonucleotides and oligonucleotide compositions are contacted with (e.g., brought into contact with, also referred to herein as administered or delivered to) and taken up by TILs described herein, including through passive uptake by TILs. The sdRNA can be added to the TILs as described herein during the first expansion, for example Step B, after the first expansion, for example, during Step C, before or during the second expansion, for example before or during Step D, after Step D and before harvest in Step E, during or after harvest in Step F, before or during final formulation and/or transfer to infusion Bag in Step F, as well as before any optional cryopreservation step in Step F. Moreover, sdRNA can be added after thawing from any cryopreservation step in Step F. In an embodiment, one or more sdRNAs targeting genes as described herein, including PD-1, LAG-3, TIM-3, CISH, CBL-B, CD38, HPK1, YAP1, and/or PTPN22 may be added to cell culture media comprising TILs and other agents at concentrations selected from the group consisting of 100 nM to 20 mM, 200 nM to 10 mM, 500 nm to 1 mM, 1 μM to 100 μM, and 1 μM to 100 μM. In an embodiment, one or more sdRNAs targeting genes as described herein, including PD-1, LAG-3, TIM-3, CISH, and CBL-B, may be added to cell culture media comprising TILs and other agents at amounts selected from the group consisting of 0.1 μM sdRNA/10,000 TILs/100 μL media, 0.5 μM sdRNA/10,000 TILs/100 μL media, 0.75 μM sdRNA/10,000 TILs/100 μL media, 1 μM sdRNA/10,000 TILs/100 μL media, 1.25 μM sdRNA/10,000 TILs/100 μL media, 1.5 μM sdRNA/10,000 TILs/100 μL media, 2 μM sdRNA/10,000 TILs/100 μL media, 5 μM sdRNA/10,000 TILs/100 μL media, or 10 μM sdRNA/10,000 TILs/100 μL media. In an embodiment, one or more sdRNAs targeting genes as described herein, including PD-1, LAG-3, TIM-3, CISH, CBL-B, CD38, HPK1, YAP1, and/or PTPN22 may be added to TIL cultures during the pre-REP or REP stages twice a day, once a day, every two days, every three days, every four days, every five days, every six days, or every seven days.

Oligonucleotide compositions of the invention, including sdRNA, can be contacted with TILs as described herein during the expansion process, for example by dissolving sdRNA at high concentrations in cell culture media and allowing sufficient time for passive uptake to occur. In certain embodiments, the method of the present invention comprises contacting a population of TILs with an oligonucleotide composition as described herein. In certain embodiments, the method comprises dissolving an oligonucleotide e.g., sdRNA in a cell culture media and contacting the cell culture media with a population of TILs. The TILs may be a first population, a second population and/or a third population as described herein.

In some embodiments, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art, such as those methods described in U.S. Pat. Nos. 4,897,355; 5,459,127; 5,631,237; 5,955,365; 5,976,567; 10,087,464; and 10,155,945; and Bergan, et al., Nucl. Acids Res. 1993, 21, 3567, the disclosures of each of which are incorporated by reference herein.

In some embodiments, more than one siRNA or sdRNA is used to reduce expression of a target gene. In some embodiments, one or more of PD-1, TIM-3, CBL-B, LAG-3, CISH, CD38, HPK1, YAP1, and/or PTPN22 targeting siRNA or sdRNAs are used together. In some embodiments, a PD-1 siRNA or sdRNA is used with one or more of TIM-3, CBL-B, LAG-3 and/or CISH in order to reduce expression of more than one gene target. In some embodiments, a LAG-3 siRNA or sdRNA is used in combination with a CISH targeting siRNA or sdRNA to reduce gene expression of both targets. In some embodiments, the siRNAs or sdRNAs targeting one or more of PD-1, TIM-3, CBL-B, LAG-3 and/or CISH herein are commercially available from Advirna LLC, Worcester, MA, USA or multiple other vendors.

In some embodiments, the siRNA or sdRNA targets a gene selected from the group consisting of PD-1, LAG-3, TIM-3, CTLA-4, TIGIT, CISH, TGFβR2, PKA, CBL-B, BAFF (BR3), CD38, HPK1, YAP1, PTPN22 and combinations thereof. In some embodiments, the siRNA or sdRNA targets a gene selected from the group consisting of PD-1, LAG-3, TIM-3, CTLA-4, TIGIT, CISH, TGFβR2, PKA, CBL-B, BAFF (BR3), CD38, HPK1, YAP1, PTPN22, and combinations thereof. In some embodiments, one siRNA or sdRNA targets PD-1 and another siRNA or sdRNA targets a gene selected from the group consisting of LAG-3, TIM-3, CTLA-4, TIGIT, CISH, TGFβR2, PKA, CBL-B, BAFF (BR3), CD38, HPK1, YAP1, PTPN22, and combinations thereof. In some embodiments, the siRNA or sdRNA targets a gene selected from PD-1, LAG-3, CISH, CBL-B, TIM-3, and combinations thereof. In some embodiments, the siRNA or sdRNA targets a gene selected from PD-1 and one of LAG-3, CISH, CBL-B, TIM-3, and combinations thereof. In some embodiments, one siRNA or sdRNA targets PD-1 and one siRNA or sdRNA targets LAG-3. In some embodiments, one siRNA or sdRNA targets PD-1 and one siRNA or sdRNA targets CISH. In some embodiments, one siRNA or sdRNA targets PD-1 and one siRNA or sdRNA targets CBL-B. In some embodiments, one siRNA or sdRNA targets LAG-3 and one siRNA or sdRNA targets CISH. In some embodiments, one siRNA or sdRNA targets LAG-3 and one siRNA or sdRNA targets CBL-B. In some embodiments, one siRNA or sdRNA targets CISH and one siRNA or sdRNA targets CBL-B. In some embodiments, one siRNA or sdRNA targets TIM-3 and one siRNA or sdRNA targets PD-1. In some embodiments, one siRNA or sdRNA targets TIM-3 and one siRNA or sdRNA targets LAG-3. In some embodiments, one siRNA or sdRNA targets TIM-3 and one siRNA or sdRNA targets CISH. In some embodiments, one siRNA or sdRNA targets TIM-3 and one siRNA or sdRNA targets CBL-B.

As discussed herein, embodiments of the present invention provide tumor infiltrating lymphocytes (TILs) that have been genetically modified via gene-editing to enhance their therapeutic effect. Embodiments of the present invention embrace genetic editing through nucleotide insertion (RNA or DNA) into a population of TILs for both promotion of the expression of one or more proteins and inhibition of the expression of one or more proteins, as well as combinations thereof. Embodiments of the present invention also provide methods for expanding TILs into a therapeutic population, wherein the methods comprise gene-editing the TILs. There are several gene-editing technologies that may be used to genetically modify a population of TILs, which are suitable for use in accordance with the present invention. Such methods include the methods described below as well as the viral and transposon methods described elsewhere herein. In an embodiment, a method of genetically modifying a TIL, MIL, or PBL to express a CCR may also include a modification to suppress the expression of a gene either via stable knockout of such a gene or transient knockdown of such a gene.

In an embodiment, the method comprises a method of genetically modifying a population of TILs in a first population, a second population and/or a third population as described herein. In an embodiment, a method of genetically modifying a population of TILs includes the step of stable incorporation of genes for production or inhibition (e.g., silencing) of one more proteins. In an embodiment, a method of genetically modifying a population of TILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In an embodiment, the electroporation method is a sterile electroporation method. In an embodiment, the electroporation method is a pulsed electroporation method. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In an embodiment, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained. In an embodiment, a method of genetically modifying a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of genetically modifying a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of genetically modifying a population of TILs includes the step of transfection using methods described in U.S. Pat. Nos. 5,766,902; 6,025,337; 6,410,517; 6,475,994; and 7,189,705; the disclosures of each of which are incorporated by reference herein. The TILs may be a first population, a second population and/or a third population of TILs as described herein.

According to an embodiment, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR/Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with TIL expansion methods of the present invention include CRISPR methods, TALE methods, and ZFN methods, which are described in more detail below. According to an embodiment, a method for expanding TILs into a therapeutic population may be carried out in accordance with any embodiment of the methods described herein (e.g., Gen 2) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by one or more of a CRISPR method, a TALE method or a ZFN method, in order to generate TILs that can provide an enhanced therapeutic effect. According to an embodiment, gene-edited TILs can be evaluated for an improved therapeutic effect by comparing them to non-modified TILs in vitro, e.g., by evaluating in vitro effector function, cytokine profiles, etc. compared to unmodified TILs. In certain embodiments, the method comprises gene editing a population of TILs using CRISPR, TALE and/or ZFN methods.

In some embodiments of the present invention, electroporation is used for delivery of a gene editing system, such as CRISPR, TALEN, and ZFN systems. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

A method for expanding TILs into a therapeutic population may be carried out in accordance with any embodiment of the methods described herein (e.g., Gen 2) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a CRISPR method (e.g., CRISPR/Cas9 or CRISPR/Cpf1). According to particular embodiments, the use of a CRISPR method during the TIL expansion process causes expression of one or more immune checkpoint genes to be silenced or reduced in at least a portion of the therapeutic population of TILs. Alternatively, the use of a CRISPR method during the TIL expansion process causes expression of one or more immune checkpoint genes to be enhanced in at least a portion of the therapeutic population of TILs.

CRISPR stands for clustered regularly interspaced short palindromic repeats. A method of using a CRISPR system for gene editing is also referred to herein as a CRISPR method. There are three types of CRISPR systems which incorporate RNAs and Cas proteins, and which may be used in accordance with the present invention: Types I, II, and III. The Type II CRISPR (exemplified by Cas9) is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR/Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR/Cas system can thereby be retargeted to cleave virtually any DNA sequence by redesigning the crRNA. The crRNA and tracrRNA in the native system can be simplified into a single guide RNA (sgRNA) of approximately 100 nucleotides for use in genetic engineering. The CRISPR/Cas system is directly portable to human cells by co-delivery of plasmids expressing the Cas9 endo-nuclease and the necessary crRNA components. Different variants of Cas proteins may be used to reduce targeting limitations (e.g., orthologs of Cas9, such as Cpf1).

Non-limiting examples of genes that may be silenced or inhibited by permanently gene-editing TILs via a CRISPR method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, CD38, HPK1, YAP1, and PTPN22.

Non-limiting examples of genes that may be enhanced by permanently gene-editing TILs via a CRISPR method include CCR2, CCR4, CCR5, CXCR2, CXCR3, CX3CR1, IL-2, IL12, IL-15, and IL-21, PGC1alpha, NT-PGC1alpha, CXCR1, CXCR4, CXCR5, CXCR6, CCR1, CCR6, CCR7, CCR8, CCR9, CCR10, BATF and c-Jun.

Examples of systems, methods, and compositions for altering the expression of a target gene sequence by a CRISPR method, and which may be used in accordance with embodiments of the present invention, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, the disclosures of each of which are incorporated by reference herein. Resources for carrying out CRISPR methods, such as plasmids for expressing CRISPR/Cas9 and CRISPR/Cpf1, are commercially available from companies such as GenScript.

In an embodiment, genetic modifications of populations of TILs, as described herein, may be performed using the CRISPR/Cpf1 system as described in U.S. Pat. No. 9,790,490, the disclosure of which is incorporated by reference herein.

A method for expanding TILs into a therapeutic population may be carried out in accordance with any embodiment of the methods described herein (e.g., Gen 2) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a TALE method. According to particular embodiments, the use of a TALE method during the TIL expansion process causes expression of one or more immune checkpoint genes to be silenced or reduced in at least a portion of the therapeutic population of TILs. Alternatively, the use of a TALE method during the TIL expansion process causes expression of one or more immune checkpoint genes to be enhanced in at least a portion of the therapeutic population of TILs.

TALE stands for transcription activator-like effector proteins, which include transcription activator-like effector nucleases (TALENs). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, KY, USA), and Life Technologies (Grand Island, NY, USA). TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of each of which are incorporated by reference herein.

Non-limiting examples of genes that may be silenced or inhibited by permanently gene-editing TILs via a TALE method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, CD38, HPK1, YAP1, and PTPN22.

Non-limiting examples of genes that may be enhanced by permanently gene-editing TILs via a TALE method include CCR2, CCR4, CCR5, CXCR2, CXCR3, CX3CR1, IL-2, IL12, IL-15, IL-21, PGC1alpha, NT-PGC1alpha, CXCR1, CXCR4, CXCR5, CXCR6, CCR1, CCR6, CCR7, CCR8, CCR9, CCR10, BATF and c-Jun.

Examples of systems, methods, and compositions for altering the expression of a target gene sequence by a TALE method, and which may be used in accordance with embodiments of the present invention, are described in U.S. Pat. No. 8,586,526, which is incorporated by reference herein.

A method for expanding TILs into a therapeutic population may be carried out in accordance with any embodiment of the methods described herein or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a zinc finger or zinc finger nuclease method. According to particular embodiments, the use of a zinc finger method during the TIL expansion process causes expression of one or more immune checkpoint genes to be silenced or reduced in at least a portion of the therapeutic population of TILs. Alternatively, the use of a zinc finger method during the TIL expansion process causes expression of one or more immune checkpoint genes to be enhanced in at least a portion of the therapeutic population of TILs.

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially from Sangamo Biosciences (Richmond, CA, USA) and Sigma-Aldrich (St. Louis, MO, USA).

Non-limiting examples of genes that may be silenced or inhibited by permanently gene-editing TILs via a zinc finger method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, CD38, HPK1, YAP1, and PTPN22.

Non-limiting examples of genes that may be enhanced by permanently gene-editing TILs via a zinc finger method include CCR2, CCR4, CCR5, CXCR2, CXCR3, CX3CR1, IL-2, IL12, IL-15, IL-21, PGC1alpha, NT-PGC1alpha, CXCR1, CXCR4, CXCR5, CXCR6, CCR1, CCR6, CCR7, CCR8, CCR9, CCR10, BATF and c-Jun.

Examples of systems, methods, and compositions for altering the expression of a target gene sequence by a zinc finger method, which may be used in accordance with embodiments of the present invention, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, each of which are incorporated by reference herein.

Other examples of systems, methods, and compositions for altering the expression of a target gene sequence by a zinc finger method, which may be used in accordance with embodiments of the present invention, are described in Beane, et al., Mol. Therapy, 2015, 23, 1380-1390, the disclosure of which is incorporated by reference herein.

In some embodiments, the TILs are optionally genetically engineered to include additional functionalities, including, but not limited to, a high-affinity TCR, e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). In certain embodiments, the method comprises genetically engineering a population of TILs to include a high-affinity TCR, e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). Aptly, the population of TILs may be a first population, a second population and/or a third population as described herein.

In some embodiments, the TILs of the present invention, including TILs modified to express CCRs and/or chemokine receptors, are optionally genetically engineered to express membrane-bound IL-2, IL-12, IL-15 and/or IL-21, for example as described in U.S. Patent Application Publication Nos. US 2021/0052647 A1 or US 2020/0172879 A1, the disclosures of which are incorporated by reference herein.

### E. Closed Systems for TIL Manufacturing

The present invention provides for the use of closed systems during the TIL culturing process. Such closed systems allow for preventing and/or reducing microbial contamination, allow for the use of fewer flasks, and allow for cost reductions. In some embodiments, the closed system uses two containers.

Such closed systems are well-known in the art and can be found, for example, at http://www.fda.gov/cber/guidelines.htm and https://www.fda.gov/BiologicsBloodVaccines/GuidanceComplianceRegulatorylnformation/Guidances/Blood/ucm076779.htm.

Sterile connecting devices (STCDs) produce sterile welds between two pieces of compatible tubing. This procedure permits sterile connection of a variety of containers and tube diameters. In some embodiments, the closed systems include luer lock and heat sealed systems as described in for example, Example G. In some embodiments, the closed system is accessed via syringes under sterile conditions in order to maintain the sterility and closed nature of the system. In some embodiments, a closed system as described in Example G is employed. In some embodiments, the TILs are formulated into a final product formulation container according to the methods described herein.

In some embodiments, the closed system uses one container from the time the tumor fragments are obtained until the TILs are ready for administration to the patient or cryopreserving. In some embodiments when two containers are used, the first container is a closed G-container and the population of TILs is centrifuged and transferred to an infusion bag without opening the first closed G-container. In some embodiments, when two containers are used, the infusion bag is a HypoThermosol-containing infusion bag. A closed system or closed TIL cell culture system is characterized in that once the tumor sample and/or tumor fragments have been added, the system is tightly sealed from the outside to form a closed environment free from the invasion of bacteria, fungi, and/or any other microbial contamination.

In some embodiments, the reduction in microbial contamination is between about 5% and about 100%. In some embodiments, the reduction in microbial contamination is between about 5% and about 95%. In some embodiments, the reduction in microbial contamination is between about 5% and about 90%. In some embodiments, the reduction in microbial contamination is between about 10% and about 90%. In some embodiments, the reduction in microbial contamination is between about 15% and about 85%. In some embodiments, the reduction in microbial contamination is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, about 99%, or about 100%.

The closed system allows for TIL growth in the absence and/or with a significant reduction in microbial contamination.

Moreover, pH, carbon dioxide partial pressure and oxygen partial pressure of the TIL cell culture environment each vary as the cells are cultured. Consequently, even though a medium appropriate for cell culture is circulated, the closed environment still needs to be constantly maintained as an optimal environment for TIL proliferation. To this end, it is desirable that the physical factors of pH, carbon dioxide partial pressure and oxygen partial pressure within the culture liquid of the closed environment be monitored by means of a sensor, the signal whereof is used to control a gas exchanger installed at the inlet of the culture environment, and the that gas partial pressure of the closed environment be adjusted in real time according to changes in the culture liquid so as to optimize the cell culture environment. In some embodiments, the present invention provides a closed cell culture system which incorporates at the inlet to the closed environment a gas exchanger equipped with a monitoring device which measures the pH, carbon dioxide partial pressure and oxygen partial pressure of the closed environment, and optimizes the cell culture environment by automatically adjusting gas concentrations based on signals from the monitoring device.

In some embodiments, the pressure within the closed environment is continuously or intermittently controlled. That is, the pressure in the closed environment can be varied by means of a pressure maintenance device for example, thus ensuring that the space is suitable for growth of TILs in a positive pressure state, or promoting exudation of fluid in a negative pressure state and thus promoting cell proliferation. By applying negative pressure intermittently, moreover, it is possible to uniformly and efficiently replace the circulating liquid in the closed environment by means of a temporary shrinkage in the volume of the closed environment.

In some embodiments, optimal culture components for proliferation of the TILs can be substituted or added, and including factors such as IL-2 and/or OKT3, as well as combination, can be added.

### F. Optional Cryopreservation of TILs

Either the bulk TIL population (for example the second population of TILs) or the expanded population of TILs (for example the third population of TILs) can be optionally cryopreserved. In some embodiments, cryopreservation occurs on the therapeutic TIL population. In some embodiments, cryopreservation occurs on the TILs harvested after the second expansion. In some embodiments, cryopreservation occurs on the TILs in exemplary Step F of FIGS. 1 and/or 8 (in particular, e.g., FIG. 8B and/or FIG. 8C). In some embodiments, the TILs are cryopreserved in the infusion bag. In some embodiments, the TILs are cryopreserved prior to placement in an infusion bag. In some embodiments, the TILs are cryopreserved and not placed in an infusion bag. In some embodiments, cryopreservation is performed using a cryopreservation medium. In some embodiments, the cryopreservation media contains dimethylsulfoxide (DMSO). This is generally accomplished by putting the TIL population into a freezing solution, e.g. 85% complement inactivated AB serum and 15% dimethyl sulfoxide (DMSO). The cells in solution are placed into cryogenic vials and stored for 24 hours at −80° C., with optional transfer to gaseous nitrogen freezers for cryopreservation. See, Sadeghi, et al., Acta Oncologica 2013, 52, 978-986.

When appropriate, the cells are removed from the freezer and thawed in a 37° C. water bath until approximately 4/5 of the solution is thawed. The cells are generally resuspended in complete media and optionally washed one or more times. In some embodiments, the thawed TILs can be counted and assessed for viability as is known in the art.

In a preferred embodiment, a population of TILs is cryopreserved using CS10 cryopreservation media (CryoStor 10, BioLife Solutions). In a preferred embodiment, a population of TILs is cryopreserved using a cryopreservation media containing dimethylsulfoxide (DMSO). In a preferred embodiment, a population of TILs is cryopreserved using a 1:1 (vol:vol) ratio of CS10 and cell culture media. In a preferred embodiment, a population of TILs is cryopreserved using about a 1:1 (vol:vol) ratio of CS10 and cell culture media, further comprising additional IL-2.

As discussed above, and exemplified in Steps A through E as provided in FIGS. 1 and/or 8 (in particular, e.g., FIG. 8B and/or FIG. 8C), cryopreservation can occur at numerous points throughout the TIL expansion process. In some embodiments, the expanded population of TILs after the first expansion (as provided for example, according to Step B or the expanded population of TILs after the one or more second expansions according to Step D of FIG. 1 or 8 (in particular, e.g., FIG. 8B and/or FIG. 8C) can be cryopreserved. Cryopreservation can be generally accomplished by placing the TIL population into a freezing solution, e.g., 85% complement inactivated AB serum and 15% dimethyl sulfoxide (DMSO). The cells in solution are placed into cryogenic vials and stored for 24 hours at −80° C., with optional transfer to gaseous nitrogen freezers for cryopreservation. See Sadeghi, et al., Acta Oncologica 2013, 52, 978-986. In some embodiments, the TILs are cryopreserved in 5% DMSO. In some embodiments, the TILs are cryopreserved in cell culture media plus 5% DMSO. In some embodiments, the TILs are cryopreserved according to the methods provided in Example 6.

When appropriate, the cells are removed from the freezer and thawed in a 37° C. water bath until approximately 4/5 of the solution is thawed. The cells are generally resuspended in complete media and optionally washed one or more times. In some embodiments, the thawed TILs can be counted and assessed for viability as is known in the art.

In some cases, the Step B TIL population can be cryopreserved immediately, using the protocols discussed below. Alternatively, the bulk TIL population can be subjected to Step C and Step D and then cryopreserved after Step D. Similarly, in the case where genetically modified TILs will be used in therapy, the Step B or Step D TIL populations can be subjected to genetic modifications for suitable treatments.

### G. Phenotypic Characteristics of Expanded TILs

In some embodiment, the TILs are analyzed for expression of numerous phenotype markers after expansion, including those described herein and in the Examples. In an embodiment, expression of one or more phenotypic markers is examined. In some embodiments, the phenotypic characteristics of the TILs are analyzed after the first expansion in Step B. In some embodiments, the phenotypic characteristics of the TILs are analyzed during the transition in Step C. In some embodiments, the phenotypic characteristics of the TILs are analyzed during the transition according to Step C and after cryopreservation. In some embodiments, the phenotypic characteristics of the TILs are analyzed after the second expansion according to Step D. In some embodiments, the phenotypic characteristics of the TILs are analyzed after two or more expansions according to Step D.

In some embodiments, the marker is selected from the group consisting of CD8 and CD28. In some embodiments, expression of CD8 is examined. In some embodiments, expression of CD28 is examined. In some embodiments, the expression of CD8 and/or CD28 is higher on TILs produced according the current invention process, as compared to other processes (e.g., the Gen 3 process as provided for example in FIG. 8 (in particular, e.g., FIG. 8B and/or FIG. 8C), as compared to the 2A process as provided for example in FIG. 8 (in particular, e.g., FIG. 8B and/or FIG. 8C). In some embodiments, the expression of CD8 is higher on TILs produced according the current invention process, as compared to other processes (e.g., the Gen 3 process as provided for example in FIG. 8 (in particular, e.g., FIG. 8B), as compared to the 2A process as provided for example in FIG. 8 (in particular, e.g., FIG. 8B and/or FIG. 8C). In some embodiments, the expression of CD28 is higher on TILs produced according the current invention process, as compared to other processes (e.g., the Gen 3 process as provided for example in FIG. 8 (in particular, e.g., FIG. 8B and/or FIG. 8C), as compared to the 2A process as provided for example in FIG. 8 (in particular, e.g., FIG. 8A)). In some embodiments, high CD28 expression is indicative of a younger, more persistent TIL phenotype. In an embodiment, expression of one or more regulatory markers is measured.

In an embodiment, no selection of the first population of TILs, second population of TILs, third population of TILs, or harvested TIL population based on CD8 and/or CD28 expression is performed during any of the steps for the method for expanding tumor infiltrating lymphocytes (TILs) described herein.

In some embodiments, the percentage of central memory cells is higher on TILs produced according the current invention process, as compared to other processes (e.g., the Gen 3 process as provided for example in FIG. 8 (in particular, e.g., FIG. 8B and/or FIG. 8C), as compared to the 2A process as provided for example in FIG. 8 (in particular, e.g., FIG. 8A)). In some embodiments the memory marker for central memory cells is selected from the group consisting of CCR7 and CD62L.

In some embodiments, the CD4+ and/or CD8+ TIL Memory subsets can be divided into different memory subsets. In some embodiments, the CD4+ and/or CD8+ TILs comprise the naïve (CD45RA+CD62L+) TILs. In some embodiments, the CD4+ and/or CD8+ TILs comprise the central memory (CM; CD45RA-CD62L+) TILs. In some embodiments, the CD4+ and/or CD8+ TILs comprise the effector memory (EM; CD45RA−CD62L−) TILs. In some embodiments, the CD4+ and/or CD8+ TILs comprise the, RA+ effector memory/effector (TEMRA/TEFF; CD45RA+CD62L+) TILs.

In some embodiments, the TILs express one more markers selected from the group consisting of granzyme B, perform, and granulysin. In some embodiments, the TILs express granzyme B. In some embodiments, the TILs express perforin. In some embodiments, the TILs express granulysin.

In an embodiment, restimulated TILs can also be evaluated for cytokine release, using cytokine release assays. In some embodiments, TILs can be evaluated for interferon-γ (IFN-γ) secretion. In some embodiments, the IFN-γ secretion is measured by an ELISA assay. In some embodiments, the IFN-γ secretion is measured by an ELISA assay after the rapid second expansion step, after Step D as provided in for example, FIG. 8 (in particular, e.g., FIG. 8B and/or FIG. 8C). In some embodiments, TIL health is measured by IFN-gamma (IFN-γ) secretion. In some embodiments, IFN-γ secretion is indicative of active TILs. In some embodiments, a potency assay for IFN-γ production is employed. IFN-γ production is another measure of cytotoxic potential. IFN-γ production can be measured by determining the levels of the cytokine IFN-γ in the media of TIL stimulated with antibodies to CD3, CD28, and CD137/4-1BB. IFN-γ levels in media from these stimulated TIL can be determined using by measuring IFN-γ release. In some embodiments, an increase in IFN-γ production in for example Step D in the Gen 3 process as provided in FIG. 8 (in particular, e.g., FIG. 8B and/or FIG. 8C) TILs as compared to for example Step D in the 2A process as provided in FIG. 8 (in particular, e.g., FIG. 8A) is indicative of an increase in cytotoxic potential of the Step D TILs. In some embodiments. IFN-γ secretion is increased one-fold, two-fold, three-fold, four-fold, or five-fold or more. In some embodiments, IFN-γ secretion is increased one-fold. In some embodiments, IFN-γ secretion is increased two-fold. In some embodiments, IFN-γ secretion is increased three-fold. In some embodiments, IFN-γ secretion is increased four-fold. In some embodiments, IFN-γ secretion is increased five-fold. In some embodiments, IFN-γ is measured using a Quantikine ELISA kit. In some embodiments, IFN-γ is measured in TILs ex vivo. In some embodiments, IFN-γ is measured in TILs ex vivo, including TILs produced by the methods of the present invention, including, for example FIG. 8B methods.

In some embodiments, TILs capable of at least one-fold, two-fold, three-fold, four-fold, or five-fold or more IFN-γ secretion are TILs produced by the expansion methods of the present invention, including, for example FIG. 8B and/or FIG. 8C methods. In some embodiments, TILs capable of at least one-fold more IFN-γ secretion are TILs produced by the expansion methods of the present invention, including, for example FIG. 8B and/or FIG. 8C methods. In some embodiments, TILs capable of at least two-fold more IFN-γ secretion are TILs produced by the expansion methods of the present invention, including, for example FIG. 8B and/or FIG. 8C methods. In some embodiments, TILs capable of at least three-fold more IFN-γ secretion are TILs produced by the expansion methods of the present invention, including, for example FIG. 8B and/or FIG. 8C methods. In some embodiments, TILs capable of at least four-fold more IFN-γ secretion are TILs produced by the expansion methods of the present invention, including, for example FIG. 8B and/or FIG. 8C methods. In some embodiments, TILs capable of at least five-fold more IFN-γ secretion are TILs produced by the expansion methods of the present invention, including, for example FIG. 8B and/or FIG. 8C methods.

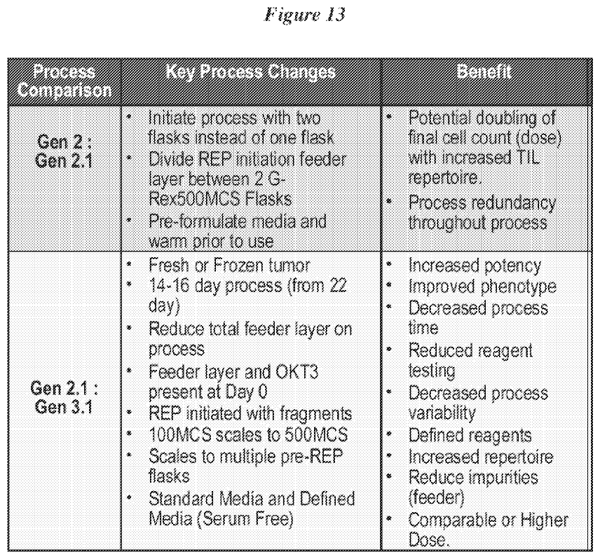

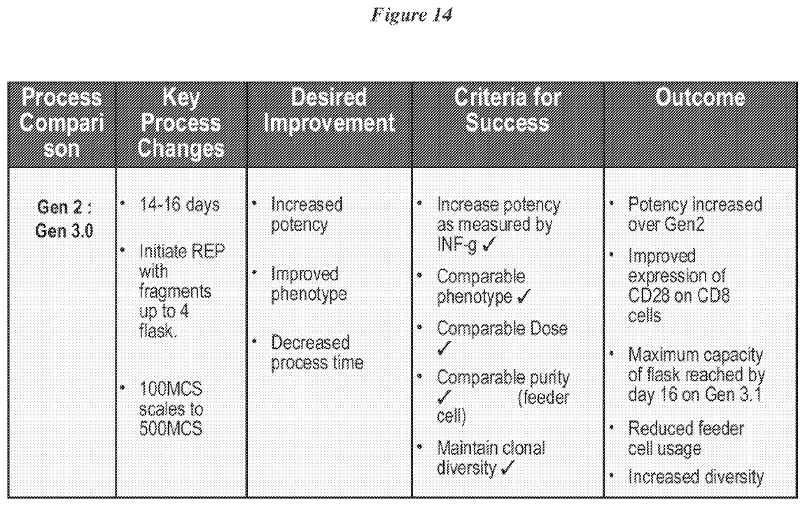

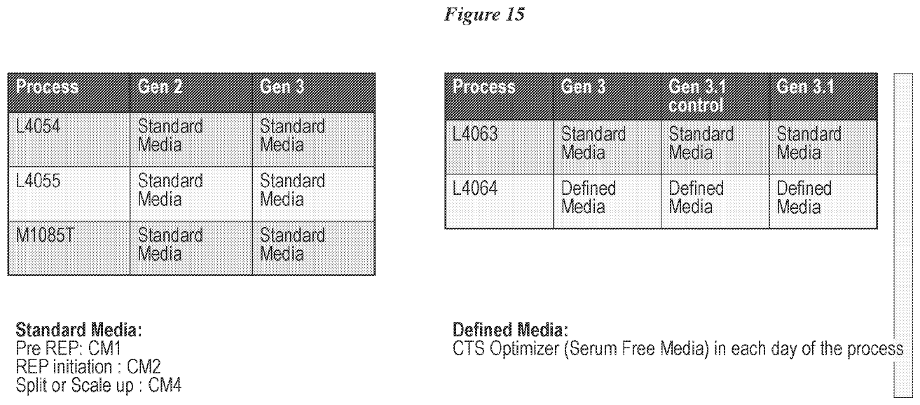

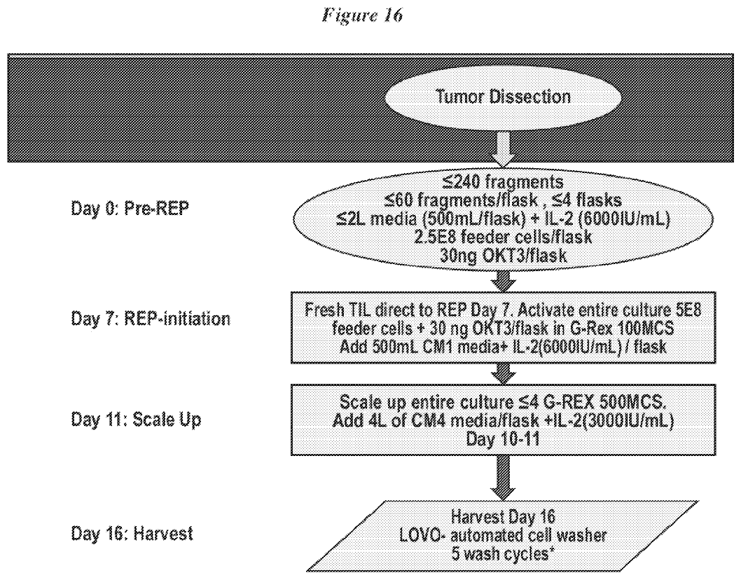

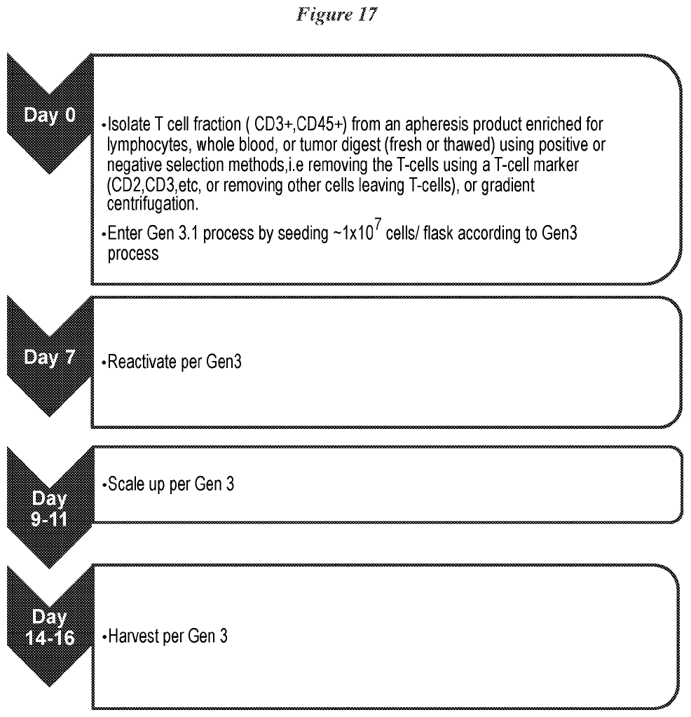

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained by the present method exhibit an increase in the T-cell repertoire diversity as compared to freshly harvested TILs and/or TILs prepared using other methods than those provide herein including, for example, methods other than those embodied in FIG. 8 (in particular, e.g., FIG. 8B and/or FIG. 8C). In some embodiments, the TILs obtained by the present method exhibit an increase in the T-cell repertoire diversity as compared to freshly harvested TILs and/or TILs prepared using methods referred to as Gen 2, as exemplified in FIG. 8 (in particular, e.g., FIG. 8A). In some embodiments, the TILs obtained in the first expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β). In some embodiments, the process as described herein (e.g., the Gen 3 process) shows higher clonal diversity as compared to other processes, for example the process referred to as the Gen 2 based on the number of unique peptide CDRs within the sample (see, for example FIGS. 12-14).

In some embodiments, the activation and exhaustion of TILs can be determined by examining one or more markers. In some embodiments, the activation and exhaustion can be determined using multicolor flow cytometry. In some embodiments, the activation and exhaustion of markers include but not limited to one or more markers selected from the group consisting of CD3, PD-1, 2B4/CD244, CD8, CD25, BTLA, KLRG, TIM-3, CD194/CCR4, CD4, TIGIT, CD183, CD69, CD95, CD127, CD103, and/or LAG-3). In some embodiments, the activation and exhaustion of markers include but not limited to one or more markers selected from the group consisting of BTLA, CTLA-4, ICOS, Ki67, LAG-3, PD-1, TIGIT, and/or TIM-3. In some embodiments, the activation and exhaustion of markers include but not limited to one or more markers selected from the group consisting of BTLA, CTLA-4, ICOS, Ki67, LAG-3. CD103+/CD69+, CD103+/CD69−, PD-1, TIGIT, and/or TIM-3. In some embodiments, the T-cell markers (including activation and exhaustion markers) can be determined and/or analyzed to examine T-cell activation, inhibition, or function. In some embodiments, the T-cell markers can include but are not limited to one or more markers selected from the group consisting of TIGIT, CD3, FoxP3, Tim-3, PD-1, CD103, CTLA-4, LAG-3, BTLA-4, ICOS, Ki67, CD8, CD25, CD45, CD4, and/or CD59.

In some embodiments, the phenotypic characterization is examined after cryopreservation.

### H. Additional Process Embodiments

In some embodiments, the invention provides a method for expanding tumor infiltrating lymphocytes (TILs) into a therapeutic population of TILs comprising: (a) obtaining a first population of TILs from a tumor resected from a subject by processing a tumor sample obtained from the subject into multiple tumor fragments; (b) performing a priming first expansion by culturing the first population of TILs in a cell culture medium comprising IL-2 and OKT-3, wherein the priming first expansion is performed for about 1 to 7 days or about 1 to 8 days to obtain the second population of TILs, wherein the second population of TILs is greater in number than the first population of TILs; (c) performing a rapid second expansion by contacting the second population of TILs with a cell culture medium comprising IL-2, OKT-3 and exogenous antigen presenting cells (APCs) to produce a third population of TILs, wherein the rapid second expansion is performed for about 1 to 11 days or about 1 to 10 days to obtain the third population of TILs, wherein the third population of TILs is a therapeutic population of TILs; and (d) harvesting the therapeutic population of TILs obtained from step (c). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 3 to 4 days, or about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 7 days, or about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days, or about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, or about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 7 days, or about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 to 7 days.

In some embodiments, the invention provides a method for expanding tumor infiltrating lymphocytes (TILs) into a therapeutic population of TILs comprising: (a) obtaining a first population of TILs from a tumor resected from a subject by processing a tumor sample obtained from the subject into multiple tumor fragments; (b) performing a priming first expansion by culturing the first population of TILs in a cell culture medium comprising IL-2 and OKT-3, wherein the priming first expansion is performed for about 1 to 8 days to obtain the second population of TILs, wherein the second population of TILs is greater in number than the first population of TILs; (c) performing a rapid second expansion by contacting the second population of TILs with a cell culture medium comprising IL-2, OKT-3 and exogenous antigen presenting cells (APCs) to produce a third population of TILs, wherein the rapid second expansion is performed for about 1 to 8 days to obtain the third population of TILs, wherein the third population of TILs is a therapeutic population of TILs; and (d) harvesting the therapeutic population of TILs obtained from step (c). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 2 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 4 to 5 days.

In some embodiments, the invention provides a method for expanding tumor infiltrating lymphocytes (TILs) into a therapeutic population of TILs comprising: (a) obtaining a first population of TILs from a tumor resected from a subject by processing a tumor sample obtained from the subject into multiple tumor fragments; (b) performing a priming first expansion by culturing the first population of TILs in a cell culture medium comprising IL-2 and OKT-3, wherein the priming first expansion is performed for about 1 to 7 days to obtain the second population of TILs, wherein the second population of TILs is greater in number than the first population of TILs; (c) performing a rapid second expansion by contacting the second population of TILs with a cell culture medium comprising IL-2, OKT-3 and exogenous antigen presenting cells (APCs) to produce a third population of TILs, wherein the rapid second expansion is performed for about 1 to 11 days to obtain the third population of TILs, wherein the third population of TILs is a therapeutic population of TILs; and (d) harvesting the therapeutic population of TILs obtained from step (c). In some embodiments, the step of rapid second expansion is split into a plurality of steps to achieve a scaling up of the culture by: (1) performing the rapid second expansion by culturing the second population of TILs in a small scale culture in a first container, e.g., a G-REX 100MCS container, for a period of about 3 to 4 days, and then (2) effecting the transfer of the second population of TILs from the small scale culture to a second container larger than the first container, e.g., a G-REX 500MCS container, wherein in the second container the second population of TILs from the small scale culture is cultured in a larger scale culture for a period of about 4 to 7 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the second population of TILs transferred from the small scale culture to such second container is cultured in a larger scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX 100MCS container, for a period of about 4 days, and then (2) effecting the transfer and apportioning of the second population of TILs from the first small scale culture into and amongst 2, 3 or 4 second containers that are larger in size than the first container, e.g., G-REX 500MCS containers, wherein in each second container the portion of the second population of TILs transferred from the small scale culture to such second container is cultured in a larger scale culture for a period of about 5 days.

In another embodiment, the invention provides the method described in any of the preceding paragraphs as applicable above modified such that in step (b) the primary first expansion is performed by contacting the first population of TILs with a culture medium which further comprises exogenous antigen-presenting cells (APCs), wherein the number of APCs in the culture medium in step (c) is greater than the number of APCs in the culture medium in step (b).

In another embodiment, the invention provides the method described in any of the preceding paragraphs as applicable above modified such that in step (c) the culture medium is supplemented with additional exogenous APCs.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 20:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 10:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 9:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 8:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 7:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 6:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 5:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 4:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 3:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.9:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.8:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.7:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.6:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.5:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.4:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.3:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.2:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2.1:1.

In another embodiment, the invention provides the method described in any of the preceding paragraphs as applicable above modified such that the ratio of number of APCs added in the rapid second expansion to the number of APCs added in step (b) is selected from a range of from at or about 1.1:1 to at or about 2:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 10:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 5:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 4:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 3:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.9:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.8:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.7:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.6:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.5:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.4:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.3:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.2:1.

In another embodiment, the invention provides the method described in any of the preceding paragraphs as applicable above modified such that the ratio of number of APCs added in the rapid second expansion to the number of APCs added in step (b) is selected from a range of from at or about 2:1 to at or about 2.1:1.

In another embodiment, the invention provides the method described in any of the preceding paragraphs as applicable above modified such that the ratio of number of APCs added in the rapid second expansion to the number of APCs added in step (b) is at or about 2:1.

In another embodiment, the invention provides the method described in any of the preceding paragraphs as applicable above modified such that the ratio of number of APCs added in the rapid second expansion to the number of APCs added in step (b) is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.61, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In another embodiment, the invention provides the method described in any of the preceding paragraphs as applicable above modified such that the number of APCs added in the primary first expansion is at or about 1×10⁸, 1.1×10, 1.2×10⁸, 1.3×10⁸,1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸,2.7×10⁸, 2.8×10⁸,2.9×10⁸, 3×10⁸, 3.1×10⁸, 3.2×10⁸, 3.3×10⁸, 3.4×10⁸ or 3.5×10⁸ APCs, and such that the number of APCs added in the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs.

In another embodiment, the invention provides the method described in any of the preceding paragraphs as applicable above modified such that the number of APCs added in the primary first expansion is selected from the range of at or about 1×10⁸ APCs to at or about 3.5×10⁸ APCs, and wherein the number of APCs added in the rapid second expansion is selected from the range of at or about 3.5×10⁸ APCs to at or about 1×10⁹ APCs.

In another embodiment, the invention provides the method described in any of the preceding paragraphs as applicable above modified such that the number of APCs added in the primary first expansion is selected from the range of at or about 1.5×10⁸ APCs to at or about 3×10⁸ APCs, and wherein the number of APCs added in the rapid second expansion is selected from the range of at or about 4×10⁸ APCs to at or about 7.5×10⁸ APCs.

In another embodiment, the invention provides the method described in any of the preceding paragraphs as applicable above modified such that the number of APCs added in the primary first expansion is selected from the range of at or about 2×10⁸ APCs to at or about 2.5×10⁸ APCs, and wherein the number of APCs added in the rapid second expansion is selected from the range of at or about 4.5×10⁸ APCs to at or about 5.5×10⁸ APCs.

In another embodiment, the invention provides the method described in any of the preceding paragraphs as applicable above modified such that at or about 2.5×10⁸ APCs are added to the primary first expansion and at or about 5×10⁸ APCs are added to the rapid second expansion.

In another embodiment, the invention provides the method described in any of the preceding paragraphs as applicable above modified such that the antigen-presenting cells are peripheral blood mononuclear cells (PBMCs).

In another embodiment, the invention provides the method described in any of the preceding paragraphs as applicable above modified such that the multiple tumor fragments are distributed into a plurality of separate containers, in each of which separate containers the first population of TILs is obtained in step (a), the second population of TILs is obtained in step (b), and the third population of TILs is obtained in step (c), and the therapeutic populations of TILs from the plurality of containers in step (c) are combined to yield the harvested TIL population from step (d).

In another embodiment, the invention provides the method described in any of the preceding paragraphs as applicable above modified such that the multiple tumors are evenly distributed into the plurality of separate containers.

In another embodiment, the invention provides the method described in any of the preceding paragraphs as applicable above modified such that the plurality of separate containers comprises at least two separate containers.

In another embodiment, the invention provides the method described in any of the preceding paragraphs as applicable above modified such that the plurality of separate containers comprises from two to twenty separate containers.

In another embodiment, the invention provides the method described in any of the preceding paragraphs as applicable above modified such that the plurality of separate containers comprises from two to fifteen separate containers.

In another embodiment, the invention provides the method described in any of the preceding paragraphs as applicable above modified such that the plurality of separate containers comprises from two to ten separate containers.

In another embodiment, the invention provides the method described in any of the preceding paragraphs as applicable above modified such that the plurality of separate containers comprises from two to five separate containers.

In another embodiment, the invention provides the method described in any of the preceding paragraphs as applicable above modified such that the plurality of separate containers comprises 2, 3, 4, 5, 6, 7, 8, 9, 10, 11, 12, 13, 14, 15, 16, 17, 18, 19, or 20 separate containers.

In another embodiment, the invention provides the method described in any of the preceding paragraphs as applicable above modified such that for each container in which the priming first expansion is performed on a first population of TILs in step (b) the rapid second expansion in step (c) is performed in the same container on the second population of TILs produced from such first population of TILs.

In another embodiment, the invention provides the method described in any of the preceding paragraphs as applicable above modified such that each of the separate containers comprises a first gas-permeable surface area.

In another embodiment, the invention provides the method described in any of the preceding paragraphs as applicable above modified such that the multiple tumor fragments are distributed in a single container.

In another embodiment, the invention provides the method described in any of the preceding paragraphs as applicable above modified such that the single container comprises a first gas-permeable surface area.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In another embodiment, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In another embodiment, the invention provides the method described in any of the preceding paragraphs as applicable above modified such that in step (b) the APCs are layered onto the first gas-permeable surface area at an average thickness of at or about 2 cell layers.

In another embodiment, the invention provides the method described in any of the preceding paragraphs as applicable above modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In another embodiment, the invention provides the method described in any of the preceding paragraphs as applicable above modified such that in step (c) the APCs are layered onto the first gas-permeable surface area at an average thickness of at or about 3 cell layers to at or about 10 cell layers.

In another embodiment, the invention provides the method described in any of the preceding paragraphs as applicable above modified such that in step (c) the APCs are layered onto the first gas-permeable surface area at an average thickness of at or about 4 cell layers to at or about 8 cell layers.

In another embodiment, the invention provides the method described in any of the preceding paragraphs as applicable above modified such that in step (c) the APCs are layered onto the first gas-permeable surface area at an average thickness of at or about 3, 4, 5, 6, 7, 8, 9 or 10 cell layers.

In another embodiment, the invention provides the method described in any of the preceding paragraphs as applicable above modified such that in step (c) the APCs are layered onto the first gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In another embodiment, the invention provides the method described in any of the preceding paragraphs as applicable above modified such that in step (b) the priming first expansion is performed in a first container comprising a first gas-permeable surface area and in step (c) the rapid second expansion is performed in a second container comprising a second gas-permeable surface area.

In another embodiment, the invention provides the method described in any of the preceding paragraphs as applicable above modified such that the second container is larger than the first container.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In another embodiment, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In another embodiment, the invention provides the method described in any of the preceding paragraphs as applicable above modified such that in step (b) the APCs are layered onto the first gas-permeable surface area at an average thickness of at or about 2 cell layers.

In another embodiment, the invention provides the method described in any of the preceding paragraphs as applicable modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In another embodiment, the invention provides the method described in any of the preceding paragraphs as applicable above modified such that in step (c) the APCs are layered onto the second gas-permeable surface area at an average thickness of at or about 3 cell layers to at or about 10 cell layers.

In another embodiment, the invention provides the method described in any of the preceding paragraphs as applicable above modified such that in step (c) the APCs are layered onto the second gas-permeable surface area at an average thickness of at or about 4 cell layers to at or about 8 cell layers.

In another embodiment, the invention provides the method described in any of the preceding paragraphs as applicable above modified such that in step (c) the APCs are layered onto the second gas-permeable surface area at an average thickness of at or about 3, 4, 5, 6, 7, 8, 9 or 10 cell layers.

In another embodiment, the invention provides the method described in any of the preceding paragraphs as applicable modified such that in step (c) the APCs are layered onto the second gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In another embodiment, the invention provides the method described in any of the preceding paragraphs as applicable above modified such that in step (b) the priming first expansion is performed in a first container comprising a first gas-permeable surface area and in step (c) the rapid second expansion is performed in the first container.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell laver to at or about three cell layers.

In another embodiment, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In another embodiment, the invention provides the method described in any of the preceding paragraphs as applicable above modified such that in step (b) the APCs are layered onto the first gas-permeable surface area at an average thickness of at or about 2 cell layers.

In another embodiment, the invention provides the method described any of the preceding paragraphs as applicable above modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In another embodiment, the invention provides the method described in any of the preceding paragraphs as applicable above modified such that in step (c) the APCs are layered onto the first gas-permeable surface area at an average thickness of at or about 3 cell layers to at or about 10 cell layers.

In another embodiment, the invention provides the method described in any of the preceding paragraphs as applicable above modified such that in step (c) the APCs are layered onto the first gas-permeable surface area at an average thickness of at or about 4 cell layers to at or about 8 cell layers.

In another embodiment, the invention provides the method described in any of the preceding paragraphs as applicable above modified such that in step (c) the APCs are layered onto the first gas-permeable surface area at an average thickness of at or about 3, 4, 5, 6, 7, 8, 9 or 10 cell layers.

In another embodiment, the invention provides the method described in any of the preceding paragraphs as applicable above modified such that in step (c) the APCs are layered onto the first gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:10.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:9.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:8.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:7.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:6.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:5.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:4.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:3.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:2.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.2 to at or about 1:8.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.3 to at or about 1:7.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.4 to at or about 1:6.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.5 to at or about 1:5.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.6 to at or about 1:4.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.7 to at or about 1:3.5.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.8 to at or about 1:3.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.9 to at or about 1:2.5.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:2.

In another embodiment, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from at or about 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9 or 1:10.

In another embodiment, the invention provides the method described in any of the preceding paragraphs as applicable above modified such that the ratio of the number of TILs in the second population of TILs to the number of TILs in the first population of TILs is at or about 1.5:1 to at or about 100:1.

In another embodiment, the invention provides the method described in any of the preceding paragraphs as applicable above modified such that the ratio of the number of TILs in the second population of TILs to the number of TILs in the first population of TILs is at or about 50:1.

In another embodiment, the invention provides the method described in any of the preceding paragraphs as applicable above modified such that the ratio of the number of TILs in the second population of TILs to the number of TILs in the first population of TILs is at or about 25:1.

In another embodiment, the invention provides the method described in any of the preceding paragraphs as applicable above modified such that the ratio of the number of TILs in the second population of TILs to the number of TILs in the first population of TILs is at or about 20:1.

In another embodiment, the invention provides the method described in any of the preceding paragraphs as applicable above modified such that the ratio of the number of TILs in the second population of TILs to the number of TILs in the first population of TILs is at or about 10:1.

In another embodiment, the invention provides the method described in any of the preceding paragraphs as applicable above modified such that the second population of TILs is at least at or about 50-fold greater in number than the first population of TILs.

In another embodiment, the invention provides the method described in any of the preceding paragraphs as applicable above modified such that the second population of TILs is at least at or about 1-, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49- or 50-fold greater in number than the first population of TILs.

In another embodiment, the invention provides the method described in any of the preceding paragraphs as applicable above modified such that at or about 2 days or at or about 3 days after the commencement of the second period in step (c), the cell culture medium is supplemented with additional IL-2.

In another embodiment, the invention provides the method described in any of the preceding paragraphs as applicable above modified to further comprise the step of cryopreserving the harvested TIL population in step (d) using a cryopreservation process.

In another embodiment, the invention provides the method described in any of the preceding paragraphs as applicable above modified to comprise performing after step (d) the additional step of (e) transferring the harvested TIL population from step (d) to an infusion bag that optionally contains HypoThermosol.

In another embodiment, the invention provides the method described in any of the preceding paragraphs as applicable above modified to comprise the step of cryopreserving the infusion bag comprising the harvested TIL population in step (e) using a cryopreservation process.

In another embodiment, the invention provides the method described in any of the preceding paragraphs as applicable above modified such that the cryopreservation process is performed using a 1:1 ratio of harvested TIL population to cryopreservation media.

In another embodiment, the invention provides the method described in any of the preceding paragraphs as applicable above modified such that the antigen-presenting cells are peripheral blood mononuclear cells (PBMCs).

In another embodiment, the invention provides the method described in any of the preceding paragraphs as applicable above modified such that the PBMCs are irradiated and allogeneic.

In another embodiment, the invention provides the method described in any of the preceding paragraphs as applicable above modified such that the total number of APCs added to the cell culture in step (b) is 2.5×10⁸.

In another embodiment, the invention provides the method described in any of the preceding paragraphs as applicable above modified such that the total number of APCs added to the cell culture in step (c) is 5×10⁸.

In another embodiment, the invention provides the method described in any of the preceding paragraphs as applicable above modified such that the APCs are PBMCs.

In another embodiment, the invention provides the method described in any of the preceding paragraphs as applicable above modified such that the PBMCs are irradiated and allogeneic.

In another embodiment, the invention provides the method described in any of the preceding paragraphs as applicable above modified such that the antigen-presenting cells are artificial antigen-presenting cells.

In another embodiment, the invention provides the method described in any of the preceding paragraphs as applicable above modified such that the harvesting in step (d) is performed using a membrane-based cell processing system.

In another embodiment, the invention provides the method described in any of the preceding paragraphs as applicable above modified such that the harvesting in step (d) is performed using a LOVO cell processing system.

In another embodiment, the invention provides the method described in any of the preceding paragraphs as applicable above modified such that the multiple fragments comprise at or about 5 to at or about 60 fragments per container in step (b).

In another embodiment, the invention provides the method described in any of the preceding paragraphs as applicable above modified such that the multiple fragments comprise at or about 10 to at or about 60 fragments per container in step (b).

In another embodiment, the invention provides the method described in any of the preceding paragraphs as applicable above modified such that the multiple fragments comprise at or about 15 to at or about 60 fragments per container in step (b).

In another embodiment, the invention provides the method described in any of the preceding paragraphs as applicable above modified such that the multiple fragments comprise at or about 20 to at or about 60 fragments per container in step (b).

In another embodiment, the invention provides the method described in any of the preceding paragraphs as applicable above modified such that the multiple fragments comprise at or about 25 to at or about 60 fragments per container in step (b).

In another embodiment, the invention provides the method described in any of the preceding paragraphs as applicable above modified such that the multiple fragments comprise at or about 30 to at or about 60 fragments per container in step (b).

In another embodiment, the invention provides the method described in any of the preceding paragraphs as applicable above modified such that the multiple fragments comprise at or about 35 to at or about 60 fragments per container in step (b).

In another embodiment, the invention provides the method described in any of the preceding paragraphs as applicable above modified such that the multiple fragments comprise at or about 40 to at or about 60 fragments per container in step (b).

In another embodiment, the invention provides the method described in any of the preceding paragraphs as applicable above modified such that the multiple fragments comprise at or about 45 to at or about 60 fragments per container in step (b).

In another embodiment, the invention provides the method described in any of the preceding paragraphs as applicable above modified such that the multiple fragments comprise at or about 50 to at or about 60 fragments per container in step (b).

In another embodiment, the invention provides the method described in any of the preceding paragraphs as applicable above modified such that the multiple fragments comprise at or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 fragment(s) per container in step (b).

In another embodiment, the invention provides the method described in any of the preceding paragraphs as applicable above modified such that each fragment has a volume of at or about 27 mm³.

In another embodiment, the invention provides the method described in any of the preceding paragraphs as applicable above modified such that each fragment has a volume of at or about 20 mm³ to at or about 50 mm³.

In another embodiment, the invention provides the method described in any of the preceding paragraphs as applicable above modified such that each fragment has a volume of at or about 21 mm³ to at or about 30 mm³.

In another embodiment, the invention provides the method described in any of the preceding paragraphs as applicable above modified such that each fragment has a volume of at or about 22 mm³ to at or about 29.5 mm³.

In another embodiment, the invention provides the method described in any of the preceding paragraphs as applicable above modified such that each fragment has a volume of at or about 23 mm³ to at or about 29 mm³.

In another embodiment, the invention provides the method described in any of the preceding paragraphs as applicable above modified such that each fragment has a volume of at or about 24 mm³ to at or about 28.5 mm³.

In another embodiment, the invention provides the method described in any of the preceding paragraphs as applicable above modified such that each fragment has a volume of at or about 25 mm³ to at or about 28 mm³.

In another embodiment, the invention provides the method described in any of the preceding paragraphs as applicable above modified such that each fragment has a volume of at or about 26.5 mm³ to at or about 27.5 mm³.

In another embodiment, the invention provides the method described in any of the preceding paragraphs as applicable above modified such that each fragment has a volume of at or about 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mm³.

In another embodiment, the invention provides the method described in any of the preceding paragraphs as applicable above modified such that the multiple fragments comprise at or about 30 to at or about 60 fragments with a total volume of at or about 1300 mm³ to at or about 1500 mm³.

In another embodiment, the invention provides the method described in any of the preceding paragraphs as applicable above modified such that the multiple fragments comprise at or about 50 fragments with a total volume of at or about 1350 mm³.

In another embodiment, the invention provides the method described in any of the preceding paragraphs as applicable above modified such that the multiple fragments comprise at or about 50 fragments with a total mass of at or about 1 gram to at or about 1.5 grams.

In another embodiment, the invention provides the method described in any of the preceding paragraphs as applicable above modified such that the cell culture medium is provided in a container that is a G-container or a Xuri cellbag.

In another embodiment, the invention provides the method described in any of the preceding paragraphs as applicable above modified such that the IL-2 concentration in the cell culture medium is about 10,000 IU/mL to about 5,000 IU/mL.

In another embodiment, the invention provides the method described in any of the preceding paragraphs as applicable above modified such that the IL-2 concentration in the cell culture medium is about 6,000 IU/mL.

In another embodiment, the invention provides the method described in any of the preceding paragraphs as applicable above modified such that the cryopreservation media comprises dimethlysulfoxide (DMSO).

In another embodiment, the invention provides the method described in any of the preceding paragraphs as applicable above modified such that the cryopreservation media comprises 7% to 10% DMSO.

In another embodiment, the invention provides the method described in any of the preceding paragraphs as applicable above modified such that the first period in step (b) is performed within a period of at or about 1 day, 2 days, 3 days, 4 days, 5 days, 6 days, or 7 days.

In another embodiment, the invention provides the method described in any of the preceding paragraphs as applicable above modified such that the second period in step (c) is performed within a period of at or about 1 day, 2 days, 3 days, 4 days, 5 days, 6 days, 7 days, 8 days, 9 days, 10 days or 11 days.

In another embodiment, the invention provides the method described in any of the preceding paragraphs as applicable above modified such that the first period in step (b) and the second period in step (c) are each individually performed within a period of at or about 1 day, 2 days, 3 days, 4 days, 5 days, 6 days, or 7 days.

In another embodiment, the invention provides the method described in any of the preceding paragraphs as applicable above modified such that the first period in step (b) and the second period in step (c) are each individually performed within a period of at or about 5 days, 6 days, or 7 days.

In another embodiment, the invention provides the method described in any of the preceding paragraphs as applicable above modified such that the first period in step (b) and the second period in step (c) are each individually performed within a period of at or about 7 days.

In another embodiment, the invention provides the method described in any of the preceding paragraphs as applicable above modified such that steps (a) through (d) are performed in a total of at or about 14 days to at or about 18 days.

In another embodiment, the invention provides the method described in any of the preceding paragraphs as applicable above modified such that steps (a) through (d) are performed in a total of at or about 15 days to at or about 18 days.

In another embodiment, the invention provides the method described in any of the preceding paragraphs as applicable above modified such that steps (a) through (d) are performed in a total of at or about 16 days to at or about 18 days.

In another embodiment, the invention provides the method described in any of the preceding paragraphs as applicable above modified such that steps (a) through (d) are performed in a total of at or about 17 days to at or about 18 days.

In another embodiment, the invention provides the method described in any of the preceding paragraphs as applicable above modified such that steps (a) through (d) are performed in a total of at or about 14 days to at or about 17 days.

In another embodiment, the invention provides the method described in any of the preceding paragraphs as applicable above modified such that steps (a) through (d) are performed in a total of at or about 15 days to at or about 17 days.

In another embodiment, the invention provides the method described in any of the preceding paragraphs as applicable above modified such that steps (a) through (d) are performed in a total of at or about 16 days to at or about 17 days.

In another embodiment, the invention provides the method described in any of the preceding paragraphs as applicable above modified such that steps (a) through (d) are performed in a total of at or about 14 days to at or about 16 days.

In another embodiment, the invention provides the method described in any of the preceding paragraphs as applicable above modified such that steps (a) through (d) are performed in a total of at or about 15 days to at or about 16 days.

In another embodiment, the invention provides the method described in any of the preceding paragraphs as applicable above modified such that steps (a) through (d) are performed in a total of at or about 14 days.

In another embodiment, the invention provides the method described in any of the preceding paragraphs as applicable above modified such that steps (a) through (d) are performed in a total of at or about 15 days.

In another embodiment, the invention provides the method described in any of the preceding paragraphs as applicable above modified such that steps (a) through (d) are performed in a total of at or about 16 days.

In another embodiment, the invention provides the method described in any of the preceding paragraphs as applicable above modified such that steps (a) through (d) are performed in a total of at or about 17 days.

In another embodiment, the invention provides the method described in any of the preceding paragraphs as applicable above modified such that steps (a) through (d) are performed in a total of at or about 18 days.

In another embodiment, the invention provides the method described in any of the preceding paragraphs as applicable above modified such that steps (a) through (d) are performed in a total of at or about 14 days or less.

In another embodiment, the invention provides the method described in any of the preceding paragraphs as applicable above modified such that steps (a) through (d) are performed in a total of at or about 15 days or less.

In another embodiment, the invention provides the method described in any of the preceding paragraphs as applicable above modified such that steps (a) through (d) are performed in a total of at or about 16 days or less.

In another embodiment, the invention provides the method described in any of the preceding paragraphs as applicable above modified such that steps (a) through (d) are performed in a total of at or about 18 days or less.

In another embodiment, the invention provides the method described in any of the preceding paragraphs as applicable above modified such that the therapeutic population of TILs harvested in step (d) comprises sufficient TILs for a therapeutically effective dosage of the TILs.

In another embodiment, the invention provides the method described in any of the preceding paragraphs as applicable above modified such that the number of TILs sufficient for a therapeutically effective dosage is from at or about 2.3×10¹⁰ to at or about 13.7×10¹⁰.

In another embodiment, the invention provides the method described in any of the preceding paragraphs as applicable above modified such that the third population of TILs in step (c) provides for increased efficacy, increased interferon-gamma production, and/or increased polyclonality.

In another embodiment, the invention provides the method described in any of the preceding paragraphs as applicable above modified such that the third population of TILs in step (c) provides for at least a one-fold to five-fold or more interferon-gamma production as compared to TILs prepared by a process longer than 16 days.

In another embodiment, the invention provides the method described in any of the preceding paragraphs as applicable above modified such that the third population of TILs in step (c) provides for at least a one-fold to five-fold or more interferon-gamma production as compared to TILs prepared by a process longer than 17 days.

In another embodiment, the invention provides the method described in any of the preceding paragraphs as applicable above modified such that the third population of TILs in step (c) provides for at least a one-fold to five-fold or more interferon-gamma production as compared to TILs prepared by a process longer than 18 days.

In another embodiment, the invention provides the method described in any of the preceding paragraphs as applicable above modified such that the effector T cells and/or central memory T cells obtained from the third population of TILs step (c) exhibit increased CD8 and CD28 expression relative to effector T cells and/or central memory T cells obtained from the second population of cells step (b).

In another embodiment, the invention provides the method described in any of the preceding paragraphs as applicable above modified such that each container recited in the method is a closed container.

In another embodiment, the invention provides the method described in any of the preceding paragraphs as applicable above modified such that each container recited in the method is a G-container.

In another embodiment, the invention provides the method described in any of the preceding paragraphs as applicable above modified such that each container recited in the method is a GREX-10.

In another embodiment, the invention provides the method described in any of the preceding paragraphs as applicable above modified such that each container recited in the method is a GREX-100.

In another embodiment, the invention provides the method described in any of the preceding paragraphs as applicable above modified such that each container recited in the method is a GREX-500.

In another embodiment, the invention provides the therapeutic population of tumor infiltrating lymphocytes (TILs) made by the method described in any of the preceding paragraphs as applicable above.

In another embodiment, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added antigen-presenting cells (APCs) or OKT3.

In another embodiment, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added antigen-presenting cells (APCs).

In another embodiment, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added OKT3.

In another embodiment, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed with no added antigen-presenting cells (APCs) and no added OKT3.

In another embodiment, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 16 days.

In another embodiment, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 17 days.

In another embodiment, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 18 days.

In another embodiment, the invention provides for the therapeutic population of TILs described in any of the preceding paragraphs as applicable above that provides for increased interferon-gamma production.

In another embodiment, the invention provides for the therapeutic population of TILs described in any of the preceding paragraphs as applicable above that provides for increased polyclonality.

In another embodiment, the invention provides for the therapeutic population of TILs described in any of the preceding paragraphs as applicable above that provides for increased efficacy.

In another embodiment, the invention provides for the therapeutic population of TILs described in any of the preceding paragraphs as applicable above modified such that the therapeutic population of TILs is capable of at least one-fold more interferon-gamma production as compared to TILs prepared by a process longer than 16 days. In another embodiment, the invention provides for the therapeutic population of TILs described in any of the preceding paragraphs as applicable above modified such that the therapeutic population of TILs is capable of at least one-fold more interferon-gamma production as compared to TILs prepared by a process longer than 17 days. In another embodiment, the invention provides for the therapeutic population of TILs described in any of the preceding paragraphs as applicable above modified such that the therapeutic population of TILs is capable of at least one-fold more interferon-gamma production as compared to TILs prepared by a process longer than 18 days. In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for the therapeutic population of TILs described in any of the preceding paragraphs as applicable above modified such that the therapeutic population of TILs is capable of at least two-fold more interferon-gamma production as compared to TILs prepared by a process longer than 16 days. In another embodiment, the invention provides for the therapeutic population of TILs described in any of the preceding paragraphs as applicable above modified such that the therapeutic population of TILs is capable of at least two-fold more interferon-gamma production as compared to TILs prepared by a process longer than 17 days. In another embodiment, the invention provides for the therapeutic population of TILs described in any of the preceding paragraphs as applicable above modified such that the therapeutic population of TILs is capable of at least two-fold more interferon-gamma production as compared to TILs prepared by a process longer than 18 days. In some embodiments, the TILs are rendered capable of the at least two-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for the therapeutic population of TILs described in any of the preceding paragraphs as applicable above modified such that the therapeutic population of TILs is capable of at least three-fold more interferon-gamma production as compared to TILs prepared by a process longer than 16 days. In another embodiment, the invention provides for the therapeutic population of TILs described in any of the preceding paragraphs as applicable above modified such that the therapeutic population of TILs is capable of at least three-fold more interferon-gamma production as compared to TILs prepared by a process longer than 17 days. In another embodiment, the invention provides for the therapeutic population of TILs described in any of the preceding paragraphs as applicable above modified such that the therapeutic population of TILs is capable of at least three-fold more interferon-gamma production as compared to TILs prepared by a process longer than 18 days. In some embodiments, the TILs are rendered capable of the at least three-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for a therapeutic population of tumor infiltrating lymphocytes (TILs) that is capable of at least one-fold more interferon-gamma production as compared to TILs prepared by a process in which the first expansion of TILs is performed without any added antigen-presenting cells (APCs). In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for a therapeutic population of tumor infiltrating lymphocytes (TILs) that is capable of at least one-fold more interferon-gamma production as compared to TILs prepared by a process in which the first expansion of TILs is performed without any added OKT3. In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for a therapeutic population of TILs that is capable of at least two-fold more interferon-gamma production as compared to TILs prepared by a process in which the first expansion of TILs is performed without any added APCs. In some embodiments, the TILs are rendered capable of the at least two-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for a therapeutic population of TILs that is capable of at least two-fold more interferon-gamma production as compared to TILs prepared by a process in which the first expansion of TILs is performed without any added OKT3. In some embodiments, the TILs are rendered capable of the at least two-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for a therapeutic population of TILs that is capable of at least three-fold more interferon-gamma production as compared to TILs prepared by a process in which the first expansion of TILs is performed without any added APCs. In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides for a therapeutic population of TILs that is capable of at least three-fold more interferon-gamma production as compared to TILs prepared by a process in which the first expansion of TILs is performed without any added OKT3. In some embodiments, the TILs are rendered capable of the at least three-fold more interferon-gamma production due to the expansion process described herein, for example as described in Steps A through F above or according to Steps A through F above (also as shown, for example, in FIG. 8 (in particular, e.g., FIG. 8B and/or FIG. 8C).

In another embodiment, the invention provides the method described in any of the preceding paragraphs as applicable above modified such that the tumor fragments are small biopsies (including, for example, a punch biopsy), core biopsies, core needle biopsies or fine needle aspirates.

In another embodiment, the invention provides the method described in any of the preceding paragraphs as applicable above modified such that the tumor fragments are core biopsies.

In another embodiment, the invention provides the method described in any of the preceding paragraphs as applicable above modified such that the tumor fragments are fine needle aspirates.

In another embodiment, the invention provides the method described in any of the preceding paragraphs as applicable above modified such that the tumor fragments are small biopsies (including, for example, a punch biopsy).

In another embodiment, the invention provides the method described in any of the preceding paragraphs as applicable above modified such that the tumor fragments are core needle biopsies.

In another embodiment, the invention provides the method described in any of the preceding paragraphs as applicable above modified such that (i) the method comprises obtaining the first population of TILs from one or more small biopsies (including, for example, a punch biopsy), core biopsies, core needle biopsies or fine needle aspirates of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion for a period of about 8 days, and (iv) the method comprises performing the rapid second expansion for a period of about 11 days. In some of the foregoing embodiments, the steps of the method are completed in about 22 days.

In another embodiment, the invention provides the method described in any of the preceding paragraphs as applicable above modified such that (i) the method comprises obtaining the first population of TILs from one or more small biopsies (including, for example, a punch biopsy), core biopsies, core needle biopsies or fine needle aspirates of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion for a period of about 8 days, and (iv) the method comprises performing the rapid second expansion by culturing the culture of the second population of TILs for about 5 days, splitting the culture into up to 5 subcultures and culturing the subcultures for about 6 days. In some of the foregoing embodiments, the up to 5 subcultures are each cultured in a container that is the same size or larger than the container in which the culture of the second population of TILs is commenced in the rapid second expansion. In some of the foregoing embodiments, the culture of the second population of TILs is equally divided amongst the up to 5 subcultures. In some of the foregoing embodiments, the steps of the method are completed in about 22 days.

In another embodiment, the invention provides the method described in any of the preceding paragraphs as applicable above modified such that the first population of TILs is obtained from 1 to about 20 small biopsies (including, for example, a punch biopsy), core biopsies, core needle biopsies or fine needle aspirates of tumor tissue from the subject.

In another embodiment, the invention provides the method described in any of the preceding paragraphs as applicable above modified such that the first population of TILs is obtained from 1 to about 10 small biopsies (including, for example, a punch biopsy), core biopsies, core needle biopsies or fine needle aspirates of tumor tissue from the subject.

In another embodiment, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 small biopsies (including, for example, a punch biopsy), core biopsies, core needle biopsies or fine needle aspirates of tumor tissue from the subject.

In another embodiment, the invention provides the method described in any of the preceding paragraphs as applicable above modified such that the first population of TILs is obtained from 1, 2, 3, 4, 5, 6, 7, 8, 9 or 10 small biopsies (including, for example, a punch biopsy), core biopsies, core needle biopsies or fine needle aspirates of tumor tissue from the subject.

In another embodiment, the invention provides the method described in any of the preceding paragraphs as applicable above modified such that the first population of TILs is obtained from 1 to about 20 core biopsies of tumor tissue from the subject.

In another embodiment, the invention provides the method described in any of the preceding paragraphs as applicable above modified such that the first population of TILs is obtained from 1 to about 10 core biopsies of tumor tissue from the subject.

In another embodiment, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 core biopsies of tumor tissue from the subject.

In another embodiment, the invention provides the method described in any of the preceding paragraphs as applicable above modified such that the first population of TILs is obtained from 1, 2, 3, 4, 5, 6, 7, 8, 9 or 10 core biopsies of tumor tissue from the subject.

In another embodiment, the invention provides the method described in any of the preceding paragraphs as applicable above modified such that the first population of TILs is obtained from 1 to about 20 fine needle aspirates of tumor tissue from the subject.

In another embodiment, the invention provides the method described in any of the preceding paragraphs as applicable above modified such that the first population of TILs is obtained from 1 to about 10 fine needle aspirates of tumor tissue from the subject.

In another embodiment, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 fine needle aspirates of tumor tissue from the subject.

In another embodiment, the invention provides the method described in any of the preceding paragraphs as applicable above modified such that the first population of TILs is obtained from 1, 2, 3, 4, 5, 6, 7, 8, 9 or 10 fine needle aspirates of tumor tissue from the subject.

In another embodiment, the invention provides the method described in any of the preceding paragraphs as applicable above modified such that the first population of TILs is obtained from 1 to about 20 core needle biopsies of tumor tissue from the subject.

In another embodiment, the invention provides the method described in any of the preceding paragraphs as applicable above modified such that the first population of TILs is obtained from 1 to about 10 core needle biopsies of tumor tissue from the subject.

In another embodiment, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 core needle biopsies of tumor tissue from the subject.

In another embodiment, the invention provides the method described in any of the preceding paragraphs as applicable above modified such that the first population of TILs is obtained from 1, 2, 3, 4, 5, 6, 7, 8, 9 or 10 core needle biopsies of tumor tissue from the subject.

In another embodiment, the invention provides the method described in any of the preceding paragraphs as applicable above modified such that the first population of TILs is obtained from 1 to about 20 small biopsies (including, for example, a punch biopsy) of tumor tissue from the subject.

In another embodiment, the invention provides the method described in any of the preceding paragraphs as applicable above modified such that the first population of TILs is obtained from 1 to about 10 small biopsies (including, for example, a punch biopsy) of tumor tissue from the subject.

In another embodiment, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 small biopsies (including, for example, a punch biopsy) of tumor tissue from the subject.

In another embodiment, the invention provides the method described in any of the preceding paragraphs as applicable above modified such that the first population of TILs is obtained from 1, 2, 3, 4, 5, 6, 7, 8, 9 or 10 small biopsies (including, for example, a punch biopsy) of tumor tissue from the subject.

In another embodiment, the invention provides the method described in any of the preceding paragraphs as applicable above modified such that (i) the method comprises obtaining the first population of TILs from 1 to about 10 core biopsies of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion step by culturing the first population of TILs in a culture medium comprising IL-2, OKT-3 and antigen presenting cells (APCs) for a period of about 8 days to obtain the second population of TILs, and (iv) the method comprises performing the rapid second expansion step by culturing the second population of TILs in a culture medium comprising IL-2, OKT-3 and APCs for a period of about 11 days. In some of the foregoing embodiments, the steps of the method are completed in about 22 days.

In another embodiment, the invention provides the method described in any of the preceding paragraphs as applicable above modified such that (i) the method comprises obtaining the first population of TILs from 1 to about 10 core biopsies of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion step by culturing the first population of TILs in a culture medium comprising IL-2, OKT-3 and antigen presenting cells (APCs) for a period of about 8 days to obtain the second population of TILs, and (iv) the method comprises performing the rapid second expansion by culturing the culture of the second population of TILs in a culture medium comprising IL-2, OKT-3 and APCs for about 5 days, splitting the culture into up to 5 subcultures and culturing each of the subcultures in a culture medium comprising IL-2 for about 6 days. In some of the foregoing embodiments, the up to 5 subcultures are each cultured in a container that is the same size or larger than the container in which the culture of the second population of TILs is commenced in the rapid second expansion. In some of the foregoing embodiments, the culture of the second population of TILs is equally divided amongst the up to 5 subcultures. In some of the foregoing embodiments, the steps of the method are completed in about 22 days.

In another embodiment, the invention provides the method described in any of the preceding paragraphs as applicable above modified such that (i) the method comprises obtaining the first population of TILs from 1 to about 10 core biopsies of tumor tissue from the subject, (ii) the method comprises performing the step of culturing the first population of TILs in a cell culture medium comprising 6000 IU IL-2/mL in 0.5 L of CM1 culture medium in a G-Rex 100M flask for a period of about 3 days prior to performing the step of the priming first expansion, (iii) the method comprises performing the priming first expansion by adding 0.5 L of CM1 culture medium containing 6000 IU/mL IL-2, 30 ng/mL OKT-3, and about 10⁸ feeder cells and culturing for a period of about 8 days, and (iv) the method comprises performing the rapid second expansion by (a) transferring the second population of TILs to a G-Rex 500MCS flask containing 5 L of CM2 culture medium with 3000 IU/mL IL-2, 30 ng/mL OKT-3, and 5×10⁹ feeder cells and culturing for about 5 days (b) splitting the culture into up to 5 subcultures by transferring 10⁹ TILs into each of up to 5 G-Rex 500MCS flasks containing 5 L of AIM-V medium with 3000 IU/mL IL-2, and culturing the subcultures for about 6 days. In some of the foregoing embodiments, the steps of the method are completed in about 22 days.

In another embodiment, the invention provides a method of expanding T cells comprising: (a) performing a priming first expansion of a first population of T cells obtained from a donor by culturing the first population of T cells to effect growth and to prime an activation of the first population of T cells; (b) after the activation of the first population of T cells primed in step (a) begins to decay, performing a rapid second expansion of the first population of T cells by culturing the first population of T cells to effect growth and to boost the activation of the first population of T cells to obtain a second population of T cells; and (c) harvesting the second population of T cells. In another embodiment, the step of rapid second expansion is split into a plurality of steps to achieve a scaling up of the culture by: (a) performing the rapid second expansion by culturing the first population of T cells in a small scale culture in a first container, e.g., a G-REX 100MCS container, for a period of about 3 to 4 days, and then (b) effecting the transfer of the first population of T cells from the small scale culture to a second container larger than the first container, e.g., a G-REX 500MCS container, and culturing the first population of T cells from the small scale culture in a larger scale culture in the second container for a period of about 4 to 7 days. In another embodiment, the step of rapid expansion is split into a plurality of steps to achieve a scaling out of the culture by: (a) performing the rapid second expansion by culturing the first population of T cells in a first small scale culture in a first container, e.g., a G-REX 100MCS container, for a period of about 3 to 4 days, and then (b) effecting the transfer and apportioning of the first population of T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the first population of T cells from first small scale culture transferred to such second container is cultured in a second small scale culture for a period of about 4 to 7 days. In another embodiment, the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the first population of T cells from the small scale culture transferred to such second container is cultured in a larger scale culture for a period of about 4 to 7 days. In another embodiment, the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 5 days.

In another embodiment, the invention provides the method described in any of the preceding paragraphs as applicable above modified such that the step of rapid second expansion is split into a plurality of steps to achieve a scaling up of the culture by: (a) performing the rapid second expansion by culturing the first population of T cells in a small scale culture in a first container, e.g., a G-REX 100MCS container, for a period of about 2 to 4 days, and then (b) effecting the transfer of the first population of T cells from the small scale culture to a second container larger than the first container, e.g., a G-REX 500MCS container, and culturing the first population of T cells from the small scale culture in a larger scale culture in the second container for a period of about 5 to 7 days.

In another embodiment, the invention provides the method described in any of the preceding paragraphs as applicable above modified such that the step of rapid expansion is split into a plurality of steps to achieve a scaling out of the culture by: (a) performing the rapid second expansion by culturing the first population of T cells in a first small scale culture in a first container, e.g., a G-REX 100MCS container, for a period of about 2 to 4 days, and then (b) effecting the transfer and apportioning of the first population of T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the first population of T cells from first small scale culture transferred to such second container is cultured in a second small scale culture for a period of about 5 to 7 days.

In another embodiment, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 2 to 4 days, and then (b) effecting the transfer and apportioning of the first population of T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX 500MCS containers, wherein in each second container the portion of the first population of T cells from the small scale culture transferred to such second container is cultured in a larger scale culture for a period of about 5 to 7 days.

In another embodiment, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 5 to 6 days.

In another embodiment, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 5 days.

In another embodiment, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 6 days.

In another embodiment, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX 100MCS container, for a period of about 3 to 4 days, and then (b) effecting the transfer and apportioning of the first population of T cells from the small scale culture into and amongst 2, 3 or 4 second containers that are larger in size than the first container, e.g., G-REX 500MCS containers, wherein in each second container the portion of the first population of T cells from the small scale culture transferred to such second container is cultured in a larger scale culture for a period of about 7 days.

In another embodiment, the invention provides the method described in any of the preceding paragraphs as applicable above modified such that the priming first expansion of step (a) is performed during a period of up to 7 days.

In another embodiment, the invention provides the method described in any of the preceding paragraphs as applicable above modified such that the rapid second expansion of step (b) is performed during a period of up to 8 days.

In another embodiment, the invention provides the method described in any of the preceding paragraphs as applicable above modified such that the rapid second expansion of step (b) is performed during a period of up to 9 days.

In another embodiment, the invention provides the method described in any of the preceding paragraphs as applicable above modified such that the rapid second expansion of step (b) is performed during a period of up to 10 days.

In another embodiment, the invention provides the method described in any of the preceding paragraphs as applicable above modified such that the rapid second expansion of step (b) is performed during a period of up to 11 days.

In another embodiment, the invention provides the method described in any of the preceding paragraphs as applicable above modified such that the priming first expansion in step (a) is performed during a period of 7 days and the rapid second expansion of step (b) is performed during a period of up to 9 days.

In another embodiment, the invention provides the method described in any of the preceding paragraphs as applicable above modified such that the priming first expansion in step (a) is performed during a period of 7 days and the rapid second expansion of step (b) is performed during a period of up to 10 days.

In another embodiment, the invention provides the method described in any of the preceding paragraphs as applicable above modified such that the priming first expansion in step (a) is performed during a period of 7 days or 8 days and the rapid second expansion of step (b) is performed during a period of up to 9 days.

In another embodiment, the invention provides the method described in any of the preceding paragraphs as applicable above modified such that the priming first expansion in step (a) is performed during a period of 7 days or 8 days and the rapid second expansion of step (b) is performed during a period of up to 10 days.

In another embodiment, the invention provides the method described in any of the preceding paragraphs as applicable above modified such that the priming first expansion in step (a) is performed during a period of 8 days and the rapid second expansion of step (b) is performed during a period of up to 9 days.

In another embodiment, the invention provides the method described in any of the preceding paragraphs as applicable above modified such that the priming first expansion in step (a) is performed during a period of 8 days and the rapid second expansion of step (b) is performed during a period of up to 8 days.

In another embodiment, the invention provides the method described in any of the preceding paragraphs as applicable above modified such that in step (a) the first population of T cells is cultured in a first culture medium comprising OKT-3 and IL-2.

In another embodiment, the invention provides the method described in any of the preceding paragraphs as applicable above modified such that the first culture medium comprises 4-1BB agonist, OKT-3 and IL-2.

In another embodiment, the invention provides the method described in any of the preceding paragraphs as applicable above modified such that the first culture medium comprises OKT-3, IL-2 and antigen-presenting cells (APCs).

In another embodiment, the invention provides the method described in any of the preceding paragraphs as applicable above modified such that the first culture medium comprises 4-1BB agonist, OKT-3, IL-2 and antigen-presenting cells (APCs).

In another embodiment, the invention provides the method described in any of the preceding paragraphs as applicable above modified such that in step (b) the first population of T cells is cultured in a second culture medium comprising OKT-3, IL-2 and antigen-presenting cells (APCs).

In another embodiment, the invention provides the method described in any of the preceding paragraphs as applicable above modified such that the second culture medium comprises 4-1BB agonist, OKT-3, IL-2 and antigen-presenting cells (APCs).

In another embodiment, the invention provides the method described in any of the preceding paragraphs as applicable above modified such that in step (a) the first population of T cells is cultured in a first culture medium in a container comprising a first gas-permeable surface, wherein the first culture medium comprises OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In another embodiment, the invention provides the method described in any of the preceding paragraphs as applicable above modified such that in step (a) the first population of T cells is cultured in a first culture medium in a container comprising a first gas-permeable surface, wherein the first culture medium comprises 4-1BB agonist, OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In another embodiment, the invention provides the method described in any of the preceding paragraphs as applicable above modified such that in step (a) the first population of T cells is cultured in a first culture medium in a container comprising a first gas-permeable surface, wherein the first culture medium comprises OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises 4-1BB agonist, OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In another embodiment, the invention provides the method described in any of the preceding paragraphs as applicable above modified such that in step (a) the first population of T cells is cultured in a first culture medium in a container comprising a first gas-permeable surface, wherein the first culture medium comprises 4-1BB agonist, OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises 4-1BB agonist, OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In another embodiment, the invention provides the method described in any of the preceding paragraphs as applicable above modified such that the ratio of the number of APCs in the second population of APCs to the number of APCs in the first population of APCs is about 2:1.

In another embodiment, the invention provides the method described in any of the preceding paragraphs as applicable above modified such that the number of APCs in the first population of APCs is about 2.5×10⁸ and the number of APCs in the second population of APCs is about 5×10⁸.

In another embodiment, the invention provides the method described in any of the preceding paragraphs as applicable above modified such that in step (a) the first population of APCs is layered onto the first gas-permeable surface at an average thickness of 2 layers of APCs.

In another embodiment, the invention provides the method described in any of the preceding paragraphs as applicable above modified such that in step (b) the second population of APCs is layered onto the first gas-permeable surface at an average thickness selected from the range of 4 to 8 layers of APCs.

In another embodiment, the invention provides the method described in any of the preceding paragraphs as applicable above modified such that the ratio of the average number of layers of APCs layered onto the first gas-permeable surface in step (b) to the average number of layers of APCs layered onto the first gas-permeable surface in step (a) is 2:1.

In another embodiment, the invention provides the method described in any of the preceding paragraphs as applicable above modified such that in step (a) the first population of APCs is seeded on the first gas permeable surface at a density selected from the range of at or about 1.0×10⁶ APCs/cm² to at or about 4.5×10⁶ APCs/cm².

In another embodiment, the invention provides the method described in any of the preceding paragraphs as applicable above modified such that in step (a) the first population of APCs is seeded on the first gas permeable surface at a density selected from the range of at or about 1.5×10⁶ APCs/cm² to at or about 3.5×10⁶ APCs/cm².

In another embodiment, the invention provides the method described in any of the preceding paragraphs as applicable above modified such that in step (a) the first population of APCs is seeded on the first gas permeable surface at a density selected from the range of at or about 2.0×10⁶ APCs/cm² to at or about 3.0×10⁶ APCs/cm².

In another embodiment, the invention provides the method described in any of the preceding paragraphs as applicable above modified such that in step (a) the first population of APCs is seeded on the first gas permeable surface at a density of at or about 2.0×10⁶ APCs/cm².

In another embodiment, the invention provides the method described in any of the preceding paragraphs as applicable above modified such that in step (b) the second population of APCs is seeded on the first gas permeable surface at a density selected from the range of at or about 2.5×10⁶ APCs/cm² to at or about 7.5×10⁶ APCs/cm².

In another embodiment, the invention provides the method described in any of the preceding paragraphs as applicable above modified such that in step (b) the second population of APCs is seeded on the first gas permeable surface at a density selected from the range of at or about 3.5×10⁶ APCs/cm² to at or about 6.0×10⁶ APCs/cm².

In another embodiment, the invention provides the method described in any of the preceding paragraphs as applicable above modified such that in step (b) the second population of APCs is seeded on the first gas permeable surface at a density selected from the range of at or about 4.0×10⁶ APCs/cm² to at or about 5.5×10⁶ APCs/cm².

In another embodiment, the invention provides the method described in any of the preceding paragraphs as applicable above modified such that in step (b) the second population of APCs is seeded on the first gas permeable surface at a density of at or about 4.0×10⁶ APCs/cm².

In another embodiment, the invention provides the method described in any of the preceding paragraphs as applicable above modified such that in step (a) the first population of APCs is seeded on the first gas permeable surface at a density selected from the range of at or about 1.0×10⁶ APCs/cm² to at or about 4.5×10⁶ APCs/cm² and in step (b) the second population of APCs is seeded on the first gas permeable surface at a density selected from the range of at or about 2.5×10⁶ APCs/cm² to at or about 7.5×10⁶ APCs/cm².

In another embodiment, the invention provides the method described in any of the preceding paragraphs as applicable modified such that in step (a) the first population of APCs is seeded on the first gas permeable surface at a density selected from the range of at or about 1.5×10⁶ APCs/cm² to at or about 3.5×10⁶ APCs/cm² and in step (b) the second population of APCs is seeded on the first gas permeable surface at a density selected from the range of at or about 3.5×10⁶ APCs/cm² to at or about 6.0×10⁶ APCs/cm².

In another embodiment, the invention provides the method described in any of the preceding paragraphs as applicable above modified such that in step (a) the first population of APCs is seeded on the first gas permeable surface at a density selected from the range of at or about 2.0×10⁶ APCs/cm² to at or about 3.0×10⁶ APCs/cm² and in step (b) the second population of APCs is seeded on the first gas permeable surface at a density selected from the range of at or about 4.0×10⁶ APCs/cm² to at or about 5.5×10⁶ APCs/cm².

In another embodiment, the invention provides the method described in any of the preceding paragraphs as applicable above modified such that in step (a) the first population of APCs is seeded on the first gas permeable surface at a density of at or about 2.0×10⁶ APCs/cm² and in step (b) the second population of APCs is seeded on the first gas permeable surface at a density of at or about 4.0×10⁶ APCs/cm².

In another embodiment, the invention provides the method described in any of the preceding paragraphs as applicable above modified such that the APCs are peripheral blood mononuclear cells (PBMCs).

In another embodiment, the invention provides the method described in any of the preceding paragraphs as applicable above modified such that the PBMCs are irradiated and exogenous to the donor of the first population of T cells.

In another embodiment, the invention provides the method described in any of the preceding paragraphs as applicable above modified such that the T cells are tumor infiltrating lymphocytes (TILs).

In another embodiment, the invention provides the method described in any of the preceding paragraphs as applicable above modified such that the T cells are marrow infiltrating lymphocytes (MILs).

In another embodiment, the invention provides the method described in any of the preceding paragraphs as applicable above modified such that the T cells are peripheral blood lymphocytes (PBLs).

In another embodiment, the invention provides the method described in any of the preceding paragraphs as applicable above modified such that the first population of T cells is obtained by separation from the whole blood of the donor.

In another embodiment, the invention provides the method described in any of the preceding paragraphs as applicable above modified such that the first population of T cells is obtained by separation from the apheresis product of the donor.

In another embodiment, the invention provides the method described in any of the preceding paragraphs as applicable above modified such that the first population of T cells is separated from the whole blood or apheresis product of the donor by positive or negative selection of a T cell phenotype.

In another embodiment, the invention provides the method described in any of the preceding paragraphs as applicable above modified such that the T cell phenotype is CD3+ and CD45+.

In another embodiment, the invention provides the method described in any of the preceding paragraphs as applicable above modified such that before performing the priming first expansion of the first population of T cells the T cells are separated from NK cells. In another embodiment, the T cells are separated from NK cells in the first population of T cells by removal of CD3− CD56+ cells from the first population of T cells. In another embodiment, the CD3− CD56+ cells are removed from the first population of T cells by subjecting the first population of T cells to cell sorting using a gating strategy that removes the CD3− CD56+ cell fraction and recovers the negative fraction. In another embodiment, the foregoing method is utilized for the expansion of T cells in a first population of T cells characterized by a high percentage of NK cells. In another embodiment, the foregoing method is utilized for the expansion of T cells in a first population of T cells characterized by a high percentage of CD3− CD56+ cells. In another embodiment, the foregoing method is utilized for the expansion of T cells in tumor tissue characterized by the present of a high number of NK cells. In another embodiment, the foregoing method is utilized for the expansion of T cells in tumor tissue characterized by a high number of CD3− CD56+ cells. In another embodiment, the foregoing method is utilized for the expansion of T cells in tumor tissue obtained from a patient suffering from a tumor characterized by the presence of a high number of NK cells. In another embodiment, the foregoing method is utilized for the expansion of T cells in tumor tissue obtained from a patient suffering from a tumor characterized by the presence of a high number of CD3− CD56+ cells. In another embodiment, the foregoing method is utilized for the expansion of T cells in tumor tissue obtained from a patient suffering from ovarian cancer.

In another embodiment, the invention provides the method described in any of the preceding paragraphs as applicable above modified such that at or about 1×10⁷ T cells from the first population of T cells are seeded in a container to initiate the primary first expansion culture in such container.

In another embodiment, the invention provides the method described in any of the preceding paragraphs as applicable above modified such that the first population of T cells is distributed into a plurality of containers, and in each container at or about 1×10⁷ T cells from the first population of T cells are seeded to initiate the primary first expansion culture in such container.

In another embodiment, the invention provides the method described in any of the preceding paragraphs as applicable above modified such that the second population of T cells harvested in step (c) is a therapeutic population of TILs.

In another embodiment, the invention provides the method described in any of the preceding paragraphs as applicable above modified such that the first population of T cells is obtained from one or more small biopsies (including, for example, a punch biopsy), core biopsies, core needle biopsies or fine needle aspirates of tumor tissue from the donor.

In another embodiment, the invention provides the method described in any of the preceding paragraphs as applicable above modified such that the first population of T cells is obtained from 1 to 20 small biopsies (including, for example, a punch biopsy), core biopsies, core needle biopsies or fine needle aspirates of tumor tissue from the donor.

In another embodiment, the invention provides the method described in any of the preceding paragraphs as applicable above modified such that the first population of T cells is obtained from 1 to 10 small biopsies (including, for example, a punch biopsy), core biopsies, core needle biopsies or fine needle aspirates of tumor tissue from the donor.

In another embodiment, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 small biopsies (including, for example, a punch biopsy), core biopsies, core needle biopsies or fine needle aspirates of tumor tissue from the donor.

In another embodiment, the invention provides the method described in any of the preceding paragraphs as applicable above modified such that the first population of T cells is obtained from 1, 2, 3, 4, 5, 6, 7, 8, 9, or 10 small biopsies (including, for example, a punch biopsy), core biopsies, core needle biopsies or fine needle aspirates of tumor tissue from the donor.

In another embodiment, the invention provides the method described in any of the preceding paragraphs as applicable above modified such that the first population of T cells is obtained from one or more core biopsies of tumor tissue from the donor.

In another embodiment, the invention provides the method described in any of the preceding paragraphs as applicable above modified such that the first population of T cells is obtained from 1 to 20 core biopsies of tumor tissue from the donor.

In another embodiment, the invention provides the method described in any of the preceding paragraphs as applicable above modified such that the first population of T cells is obtained from 1 to 10 core biopsies of tumor tissue from the donor.

In another embodiment, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 core biopsies of tumor tissue from the donor.

In another embodiment, the invention provides the method described in any of the preceding paragraphs as applicable above modified such that the first population of T cells is obtained from 1, 2, 3, 4, 5, 6, 7, 8, 9, or 10 core biopsies of tumor tissue from the donor.

In another embodiment, the invention provides the method described in any of the preceding paragraphs as applicable above modified such that the first population of T cells is obtained from one or more fine needle aspirates of tumor tissue from the donor.

In another embodiment, the invention provides the method described in any of the preceding paragraphs as applicable above modified such that the first population of T cells is obtained from 1 to 20 fine needle aspirates of tumor tissue from the donor.

In another embodiment, the invention provides the method described in any of the preceding paragraphs as applicable above modified such that the first population of T cells is obtained from 1 to 10 fine needle aspirates of tumor tissue from the donor.

In another embodiment, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 fine needle aspirates of tumor tissue from the donor.

In another embodiment, the invention provides the method described in any of the preceding paragraphs as applicable above modified such that the first population of T cells is obtained from 1, 2, 3, 4, 5, 6, 7, 8, 9, or 10 fine needle aspirates of tumor tissue from the donor.

In another embodiment, the invention provides the method described in any of the preceding paragraphs as applicable above modified such that the first population of T cells is obtained from one or more small biopsies (including, for example, a punch biopsy) of tumor tissue from the donor.

In another embodiment, the invention provides the method described in any of the preceding paragraphs as applicable above modified such that the first population of T cells is obtained from 1 to 20 small biopsies (including, for example, a punch biopsy) of tumor tissue from the donor.

In another embodiment, the invention provides the method described in any of the preceding paragraphs as applicable above modified such that the first population of T cells is obtained from 1 to 10 small biopsies (including, for example, a punch biopsy) of tumor tissue from the donor.

In another embodiment, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 small biopsies (including, for example, a punch biopsy) of tumor tissue from the donor.

In another embodiment, the invention provides the method described in any of the preceding paragraphs as applicable above modified such that the first population of T cells is obtained from 1, 2, 3, 4, 5, 6, 7, 8, 9, or 10 small biopsies (including, for example, a punch biopsy) of tumor tissue from the donor.

In another embodiment, the invention provides the method described in any of the preceding paragraphs as applicable above modified such that the first population of T cells is obtained from one or more core needle biopsies of tumor tissue from the donor.

In another embodiment, the invention provides the method described in any of the preceding paragraphs as applicable above modified such that the first population of T cells is obtained from 1 to 20 core needle biopsies of tumor tissue from the donor.

In another embodiment, the invention provides the method described in any of the preceding paragraphs as applicable above modified such that the first population of T cells is obtained from 1 to 10 core needle biopsies of tumor tissue from the donor.

In another embodiment, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 core needle biopsies of tumor tissue from the donor.

In another embodiment, the invention provides the method described in any of the preceding paragraphs as applicable above modified such that the first population of T cells is obtained from 1, 2, 3, 4, 5, 6, 7, 8, 9, or 10 core needle biopsies of tumor tissue from the donor.

In another embodiment, the invention provides a method for expanding tumor infiltrating lymphocytes (TILs) into a therapeutic population of TILs comprising: i) obtaining and/or receiving a first population of TILs from a tumor sample obtained from one or more small biopsies, core biopsies, or needle biopsies of a tumor in a subject by culturing the tumor sample in a first cell culture medium comprising IL-2 for about 3 days; (ii) performing a priming first expansion by culturing the first population of TILs in a second cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 7 or 8 days to obtain the second population of TILs, wherein the second population of TILs is greater in number than the first population of TILs; (iii) performing a rapid second expansion by supplementing the second cell culture medium of the second population of TILs with additional IL-2, OKT-3, and APCs, to produce a third population of TILs, wherein the number of APCs added in the rapid second expansion is at least twice the number of APCs added in step (ii), wherein the rapid second expansion is performed for a second period of about 11 days to obtain the third population of TILs, wherein the third population of TILs is a therapeutic population of TILs, wherein the rapid second expansion is performed in a container comprising a second gas-permeable surface area; (iv) harvesting the therapeutic population of TILs obtained from step (iii); and (v) transferring the harvested TIL population from step (iv) to an infusion bag.

In another embodiment, the invention provides a method for expanding tumor infiltrating lymphocytes (TILs) into a therapeutic population of TILs comprising: (i) obtaining and/or receiving a first population of TILs from a tumor sample obtained from one or more small biopsies, core biopsies, or needle biopsies of a tumor in a subject by culturing the tumor sample in a first cell culture medium comprising IL-2 for about 3 days; (ii) performing a priming first expansion by culturing the first population of TILs in a second cell culture medium comprising IL-2, OKT-3, and antigen presenting cells (APCs) to produce a second population of TILs, wherein the priming first expansion is performed for first period of about 7 or 8 days to obtain the second population of TILs, wherein the second population of TILs is greater in number than the first population of TILs; (iii) performing a rapid second expansion by contacting the second population of TILs with a third cell culture medium comprising IL-2, OKT-3, and APCs, to produce a third population of TILs, wherein the rapid second expansion is performed for a second period of about 11 days to obtain the third population of TILs, wherein the third population of TILs is a therapeutic population of TILs; and (iv) harvesting the therapeutic population of TILs obtained from step (iii).

In another embodiment, the invention provides the method described in any of the preceding paragraphs as applicable above modified such that after day 5 of the second period the culture is split into 2 or more subcultures, and each subculture is supplemented with an additional quantity of the third culture medium and cultured for about 6 days.

In another embodiment, the invention provides the method described in any of the preceding paragraphs as applicable above modified such that after day 5 of the second period the culture is split into 2 or more subcultures, and each subculture is supplemented with a fourth culture medium comprising IL-2 and cultured for about 6 days.

In another embodiment, the invention provides the method described in any of the preceding paragraphs as applicable above modified such that after day 5 of the second period the culture is split into up to 5 subcultures.

In another embodiment, the invention provides the method described in any of the preceding paragraphs as applicable above modified such that all steps in the method are completed in about 22 days.

In another embodiment, the invention provides a method of expanding T cells comprising: (i) performing a priming first expansion of a first population of T cells from a tumor sample obtained from one or more small biopsies, core biopsies, or needle biopsies of a tumor in a donor by culturing the first population of T cells to effect growth and to prime an activation of the first population of T cells; (ii) after the activation of the first population of T cells primed in step (a) begins to decay, performing a rapid second expansion of the first population of T cells by culturing the first population of T cells to effect growth and to boost the activation of the first population of T cells to obtain a second population of T cells; and (iv) harvesting the second population of T cells. In some embodiments, the tumor sample is obtained from a plurality of core biopsies. In some embodiments, the plurality of core biopsies is selected from the group consisting of 2, 3, 4, 5, 6, 7, 8, 9 and 10 core biopsies.

In some embodiments, the invention the method described in any of the preceding paragraphs as applicable above modified such that T cells or TILs are obtained from tumor digests. In some embodiments, tumor digests are generated by incubating the tumor in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). In some embodiments, the tumor is placed in a tumor dissociating enzyme mixture including one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof. In other embodiments, the tumor is placed in a tumor dissociating enzyme mixture including collagenase (including any blend or type of collagenase), neutral protease (dispase) and deoxyribonuclease I (DNase).

### VI. Pharmaceutical Compositions, Dosages, and Dosing Regimens

In an embodiment, TILs, MILs, or PBLs expanded and/or genetically modified (including TILs, MILs, or PBLs genetically-modified to express a CCR) using the methods of the present disclosure are administered to a patient as a pharmaceutical composition. In an embodiment, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. In some embodiments, from about 2.3×10¹⁰ to about 13.7×10¹⁰ TILs are administered, with an average of around 7.8×10¹⁰ TILs, particularly if the cancer is melanoma. In an embodiment, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, the therapeutically effective dosage is about 2.3×10¹⁰ to about 13.7×10¹⁰. In some embodiments, the therapeutically effective dosage is about 7.8×10¹⁰ TILs, particularly of the cancer is melanoma. In some embodiments, the therapeutically effective dosage is about 1.2×10¹⁰ to about 4.3×10¹⁰ of TILs. In some embodiments, the therapeutically effective dosage is about 3×10¹⁰ to about 12×10¹⁰ TILs. In some embodiments, the therapeutically effective dosage is about 4×10¹⁰ to about 10×10¹⁰⁰ TILs. In some embodiments, the therapeutically effective dosage is about 5×10¹⁰ to about 8×10¹⁰ TILs. In some embodiments, the therapeutically effective dosage is about 6×10¹⁰ to about 8×10¹⁰ TILs. In some embodiments, the therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² 1×10¹³, 2×10¹³, 3×10¹³, 4×10¹³, 5×10¹³, 6×10¹³, 7×10¹³, 8×10¹³, and 9×10¹³. In an embodiment, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁹ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.10% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.0010% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶ 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸,3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰ 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹ 1×10¹², 2×10¹², 3×10¹², 4×10¹², 5×10¹². 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

In another embodiment, the invention provides an infusion bag comprising the therapeutic population of TILs described in any of the preceding paragraphs above.

In another embodiment, the invention provides a tumor infiltrating lymphocyte (TIL) composition comprising the therapeutic population of TILs described in any of the preceding paragraphs above and a pharmaceutically acceptable carrier.

In another embodiment, the invention provides an infusion bag comprising the TIL composition described in any of the preceding paragraphs above.

In another embodiment, the invention provides a cryopreserved preparation of the therapeutic population of TILs described in any of the preceding paragraphs above.

In another embodiment, the invention provides a tumor infiltrating lymphocyte (TIL) composition comprising the therapeutic population of TILs described in any of the preceding paragraphs above and a cryopreservation media.

In another embodiment, the invention provides the TIL composition described in any of the preceding paragraphs above modified such that the cryopreservation media contains DMSO.

In another embodiment, the invention provides the TIL composition described in any of the preceding paragraphs above modified such that the cryopreservation media contains 7-10% DMSO.

In another embodiment, the invention provides a cryopreserved preparation of the TIL composition described in any of the preceding paragraphs above.

In an embodiment, TILs expanded using the methods of the present disclosure are administered to a patient as a pharmaceutical composition. In an embodiment, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. In some embodiments, from about 2.3×10¹⁰ to about 13.7×10¹⁰ TILs are administered, with an average of around 7.8×10¹⁰ TILs, particularly if the cancer is NSCLC. In an embodiment, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, therapeutically effective dosage is about 2.3×10¹⁰ to about 13.7×10¹⁰. In some embodiments, therapeutically effective dosage is about 7.8×10¹⁰ TILs, particularly of the cancer is NSCLC. In some embodiments, therapeutically effective dosage is about 1.2×10¹⁰ to about 4.3×10¹⁰ of TILs. In some embodiments, therapeutically effective dosage is about 3×10¹⁰ to about 12×10¹⁰ TILs. In some embodiments, therapeutically effective dosage is about 4×10¹⁰ to about 10×10¹⁰ TILs. In some embodiments, therapeutically effective dosage is about 5×10¹⁰ to about 8×10¹⁰ TILs. In some embodiments, therapeutically effective dosage is about 6×10¹⁰ to about 8×10¹⁰ TILs. In some embodiments, therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In an embodiment, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25%, 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.10% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.0010% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶ 6×10⁶, 7×10⁶ 8×10⁶, 9×10⁶, 1×10⁷, 2×10⁷ 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

### VII. Methods of Treating Patients

Methods of treatment begin with the initial TIL collection and culture of TILs, optionally modified as described herein to express one or more CCRs and/or one or more chemokine receptors. Such methods of treatment have been both described in the art by, for example, Jin, et al., J. Immunotherapy 2012, 35(3):283-292, incorporated by reference herein in its entirety. Embodiments of methods of treatment are described throughout the sections below, including the Examples.

The expanded TILs produced according the methods described herein, including for example as described in Steps A through F above (or as shown, for example, in FIG. 1) find particular use in the treatment of patients with cancer (for example, as described in Goff, et al., J. Clinical Oncology, 2016, 34(20):2389-239, as well as the supplemental content; incorporated by reference herein in its entirety. In some embodiments, TILs were grown from resected deposits of metastatic melanoma as previously described (see Dudley, et al., J Immunotherapy 2003, 26:332-342; incorporated by reference herein in its entirety). Fresh tumor can be dissected under sterile conditions. A representative sample can be collected for formal pathologic analysis. Single fragments of 2 mm³ to 3 mm³ may be used. In some embodiments, 5, 10, 15, 20, 25 or 30 samples per patient are obtained. In some embodiments, 20, 25, or 30 samples per patient are obtained. In some embodiments, 20, 22, 24, 26, or 28 samples per patient are obtained. In some embodiments, 24 samples per patient are obtained. Samples can be placed in individual wells of a 24-well plate, maintained in growth media with high-dose IL-2 (6,000 IU/mL), and monitored for destruction of tumor and/or proliferation of TIL. Any tumor with viable cells remaining after processing can be enzymatically digested into a single cell suspension and cryopreserved, as described herein.

In some embodiments, successfully grown TIL can be sampled for phenotype analysis (CD3, CD4, CD8, and CD56) and tested against autologous tumor when available. TIL can be considered reactive if overnight coculture yielded interferon-gamma (IFN-γ) levels>200 pg/mL and twice background. (Goff, et al., J Immunother., 2010, 33:840-847; incorporated by reference herein in its entirety). In some embodiments, cultures with evidence of autologous reactivity or sufficient growth patterns can be selected for a second expansion, including second expansions that are sometimes referred to as rapid expansion (REP). In some embodiments, expanded TILs with high autologous reactivity (for example, high proliferation during a second expansion), are selected for an additional second expansion. In some embodiments, TILs with high autologous reactivity are selected for an additional second REP expansion.

Cell phenotypes of cryopreserved samples of infusion bag TIL can be analyzed by flow cytometry (e.g., FlowJo) for surface markers CD3, CD4, CD8, CCR7, and CD45RA (BD BioSciences), as well as by any of the methods described herein. Serum cytokines were measured by using standard enzyme-linked immunosorbent assay techniques. A rise in serum IFN-g was defined as >100 μg/mL and greater than 4 3 baseline levels.

In some embodiments, the TILs produced by the methods provided herein, for example those exemplified herein, provide for a surprising improvement in clinical efficacy of the TILs. In some embodiments, the TILs produced by the methods provided herein, for example those exemplified in FIG. 1, exhibit increased clinical efficacy as compared to TILs produced by methods other than those described herein, including for example, methods other than those exemplified in FIG. 1. In some embodiments, the methods other than those described herein include methods referred to as process 1C and/or Generation 1 (Gen 1). In some embodiments, the increased efficacy is measured by DCR, ORR, and/or other clinical responses. In some embodiments, the TILs produced by the methods provided herein, for example those exemplified in FIG. 1, exhibit a similar time to response and safety profile compared to TILs produced by methods other than those described herein, including for example, methods other than those exemplified in FIG. 1, for example the Gen 1 process.

In some embodiments, IFN-gamma (IFN-γ) is indicative of treatment efficacy and/or increased clinical efficacy. In some embodiments, IFN-γ in the blood of subjects treated with TILs is indicative of active TILs. In some embodiments, a potency assay for IFN-γ production is employed. IFN-γ production is another measure of cytotoxic potential. IFN-γ production can be measured by determining the levels of the cytokine IFN-γ in the blood, serum, or TILs ex vivo of a subject treated with TILs prepared by the methods of the present invention, including those as described for example in FIG. 1. In some embodiments, an increase in IFN-γ is indicative of treatment efficacy in a patient treated with the TILs produced by the methods of the present invention. In some embodiments, IFN-γ is increased one-fold, two-fold, three-fold, four-fold, or five-fold or more as compared to an untreated patient and/or as compared to a patient treated with TILs prepared using other methods than those provide herein including for example, methods other than those embodied in FIG. 1. In some embodiments, IFN-γ secretion is increased one-fold as compared to an untreated patient and/or as compared to a patient treated with TILs prepared using other methods than those provide herein including for example, methods other than those embodied in FIG. 1. In some embodiments, IFN-γ secretion is increased two-fold as compared to an untreated patient and/or as compared to a patient treated with TILs prepared using other methods than those provide herein including for example, methods other than those embodied in FIG. 1. In some embodiments, IFN-γ secretion is increased three-fold as compared to an untreated patient and/or as compared to a patient treated with TILs prepared using other methods than those provide herein including for example, methods other than those embodied in FIG. 1. In some embodiments, IFN-γ secretion is increased four-fold as compared to an untreated patient and/or as compared to a patient treated with TILs prepared using other methods than those provide herein including for example, methods other than those embodied in FIG. 1. In some embodiments, IFN-γ secretion is increased five-fold as compared to an untreated patient and/or as compared to a patient treated with TILs prepared using other methods than those provide herein including for example, methods other than those embodied in FIG. 1. In some embodiments, IFN-γ is measured using a Quantikine ELISA kit. In some embodiments, IFN-γ is measured in TILs ex vivo of a subject treated with TILs prepared by the methods of the present invention, including those as described for example in FIG. 1. In some embodiments, IFN-γ is measured in blood of a subject treated with TILs prepared by the methods of the present invention, including those as described for example in FIG. 1. In some embodiments, IFN-γ is measured in TILs serum of a subject treated with TILs prepared by the methods of the present invention, including those as described for example in FIG. 1.

In some embodiments, the TILs prepared by the methods of the present invention, including those as described for example in FIG. 1, exhibit increased polyclonality as compared to TILs produced by other methods, including those not exemplified in FIG. 1, such as for example, methods referred to as process 1C methods. In some embodiments, significantly improved polyclonality and/or increased polyclonality is indicative of treatment efficacy and/or increased clinical efficacy. In some embodiments, polyclonality refers to the T-cell repertoire diversity. In some embodiments, an increase in polyclonality can be indicative of treatment efficacy with regard to administration of the TILs produced by the methods of the present invention. In some embodiments, polyclonality is increased one-fold, two-fold, ten-fold, 100-fold, 500-fold, or 1000-fold as compared to TILs prepared using methods than those provide herein including for example, methods other than those embodied in FIG. 1. In some embodiments, polyclonality is increased one-fold as compared to an untreated patient and/or as compared to a patient treated with TILs prepared using other methods than those provide herein including for example, methods other than those embodied in FIG. 1. In some embodiments, polyclonality is increased two-fold as compared to an untreated patient and/or as compared to a patient treated with TILs prepared using other methods than those provide herein including for example, methods other than those embodied in FIG. 1. In some embodiments, polyclonality is increased ten-fold as compared to an untreated patient and/or as compared to a patient treated with TILs prepared using other methods than those provide herein including for example, methods other than those embodied in FIG. 1. In some embodiments, polyclonality is increased 100-fold as compared to an untreated patient and/or as compared to a patient treated with TILs prepared using other methods than those provide herein including for example, methods other than those embodied in FIG. 1. In some embodiments, polyclonality is increased 500-fold as compared to an untreated patient and/or as compared to a patient treated with TILs prepared using other methods than those provide herein including for example, methods other than those embodied in FIG. 1. In some embodiments, polyclonality is increased 1000-fold as compared to an untreated patient and/or as compared to a patient treated with TILs prepared using other methods than those provide herein including for example, methods other than those embodied in FIG. 1.

Measures of efficacy can include the disease control rate (DCR) as well as overall response rate (ORR), as known in the art as well as described herein.

### A. Methods of Treating Cancers

The compositions and methods described herein can be used in a method for treating diseases. In an embodiment, they are for use in treating hyperproliferative disorders, such as cancer, in an adult patient or in a pediatric patient. They may also be used in treating other disorders as described herein and in the following paragraphs.

In some embodiments, the hyperproliferative disorder is cancer. In some embodiments, the hyperproliferative disorder is a solid tumor cancer. In some embodiments, the solid tumor cancer is selected from the group consisting of anal cancer, bladder cancer, breast cancer (including triple-negative breast cancer), bone cancer, cancer caused by human papilloma virus (HPV), central nervous system associated cancer (including ependymoma, medulloblastoma, neuroblastoma, pineoblastoma, and primitive neuroectodermal tumor), cervical cancer (including squamous cell cervical cancer, adenosquamous cervical cancer, and cervical adenocarcinoma), colon cancer, colorectal cancer, endometrial cancer, esophageal cancer, esophagogastric junction cancer, gastric cancer, gastrointestinal cancer, gastrointestinal stromal tumor, glioblastoma, glioma, head and neck cancer (including head and neck squamous cell carcinoma (HNSCC), hypopharynx cancer, larynx cancer, nasopharynx cancer, oropharynx cancer, and pharynx cancer), kidney cancer, liver cancer, lung cancer (including non-small-cell lung cancer (NSCLC) and small-cell lung cancer), melanoma (including uveal melanoma, choroidal melanoma, ciliary body melanoma, or iris melanoma), mesothelioma (including malignant pleural mesothelioma), ovarian cancer, pancreatic cancer (including pancreatic ductal adenocarcinoma), penile cancer, rectal cancer, renal cancer, renal cell carcinoma, sarcoma (including Ewing sarcoma, osteosarcoma, rhabdomyosarcoma, and other bone and soft tissue sarcomas), thyroid cancer (including anaplastic thyroid cancer), uterine cancer, and vaginal cancer.

In some embodiments, the hyperproliferative disorder is a hematological malignancy. In some embodiments, the hematological malignancy is selected from the group consisting of chronic lymphocytic leukemia, acute lymphoblastic leukemia, diffuse large B cell lymphoma, non-Hodgkin's lymphoma, Hodgkin's lymphoma, follicular lymphoma, mantle cell lymphoma, and multiple myeloma. In some embodiments, the present invention includes a method of treating a patient with a cancer, wherein the cancer is a hematological malignancy. In some embodiments, the present invention includes a method of treating a patient with a cancer using TILs, MILs, or PBLs modified to express one or more CCRs, wherein the cancer is a hematological malignancy. In some embodiments, the present invention includes a method of treating a patient with a cancer using MILs or PBLs modified to express one or more CCRs, wherein the cancer is a hematological malignancy.

In an embodiment, the cancer is one of the foregoing cancers, including solid tumor cancers and hematological malignancies, that is relapsed or refractory to treatment with at least one prior therapy, including chemotherapy, radiation therapy, or immunotherapy. In an embodiment, the cancer is one of the foregoing cancers that is relapsed or refractory to treatment with at least two prior therapies, including chemotherapy, radiation therapy, and/or immunotherapy. In an embodiment, the cancer is one of the foregoing cancers that is relapsed or refractory to treatment with at least three prior therapies, including chemotherapy, radiation therapy, and/or immunotherapy.

In some embodiments, the cancer is a microsatellite instability-high (MSI-H) or a mismatch repair deficient (dMMR) cancer. MSI-H and dMMR cancers and testing therefore have been described in Kawakami, et al., Curr. Treat. Options Oncol. 2015, 16, 30, the disclosures of which are incorporated by reference herein.

In some embodiments, the present invention includes a method of treating a patient with a cancer using TILs, MILs, or PBLs modified to express one or more CCRs, wherein the patient is a human. In some embodiments, the present invention includes a method of treating a patient with a cancer using TILs, MILs, or PBLs modified to express one or more CCRs, wherein the patient is a non-human. In some embodiments, the present invention includes a method of treating a patient with a cancer using TILs, MILs, or PBLs modified to express one or more CCRs, wherein the patient is a companion animal. In some embodiments, the present invention includes a method of treating a patient with a cancer using TILs, MILs, or PBLs modified to express one or more CCRs, wherein the patient is a primate, equine, canine, or feline animal.

In some embodiments, the present invention includes a method of treating a patient with a cancer, wherein the cancer is refractory to treatment with a BRAF inhibitor and/or a MEK inhibitor. In some embodiments, the present invention includes a method of treating a patient with a cancer, wherein the cancer is refractory to treatment with a BRAF inhibitor selected from the group consisting of vemurafenib, dabrafenib, encorafenib, sorafenib, and pharmaceutically acceptable salts or solvates thereof. In some embodiments, the present invention includes a method of treating a patient with a cancer, wherein the cancer is refractory to treatment with a MEK inhibitor selected from the group consisting of trametinib, cobimetinib, binimetinib, selumetinib, pimasertinib, refametinib, and pharmaceutically acceptable salts or solvates thereof. In some embodiments, the present invention includes a method of treating a patient with a cancer, wherein the cancer is refractory to treatment with a BRAF inhibitor selected from the group consisting of vemurafenib, dabrafenib, encorafenib, sorafenib, and pharmaceutically acceptable salts or solvates thereof, and a MEK inhibitor selected from the group consisting of trametinib, cobimetinib, binimetinib, selumetinib, pimasertinib, refametinib, and pharmaceutically acceptable salts or solvates thereof.

In some embodiments, the present invention includes a method of treating a patient with a cancer, wherein the cancer is a pediatric cancer.

In some embodiments, the present invention includes a method of treating a patient with a cancer wherein the cancer is uveal melanoma.

In some embodiments, the present invention includes a method of treating a patient with a cancer, wherein the uveal melanoma is choroidal melanoma, ciliary body melanoma, or iris melanoma.

In some embodiments, the present invention includes a method of treating a patient with a cancer, wherein the pediatric cancer is a neuroblastoma.

In some embodiments, the present invention includes a method of treating a patient with a cancer, wherein the pediatric cancer is a sarcoma.

In some embodiments, the present invention includes a method of treating a patient with a cancer, wherein the sarcoma is osteosarcoma.

In some embodiments, the present invention includes a method of treating a patient with a cancer, wherein the sarcoma is a soft tissue sarcoma.

In some embodiments, the present invention includes a method of treating a patient with a cancer, wherein the soft tissue sarcoma is rhabdomyosarcoma, Ewing sarcoma, or primitive neuroectodermal tumor (PNET).

In some embodiments, the present invention includes a method of treating a patient with a cancer, wherein the pediatric cancer is a central nervous system (CNS) associated cancer. In some embodiments, the pediatric cancer is refractory to treatment with chemotherapy. In some embodiments, the pediatric cancer is refractory to treatment with radiation therapy. In some embodiments, the pediatric cancer is refractory to treatment with dinutuximab.

In some embodiments, the present invention includes a method of treating a patient with a cancer, wherein the CNS associated cancer is medulloblastoma, pineoblastoma, glioma, ependymoma, or glioblastoma.

The compositions and methods described herein can be used in a method for treating cancer, wherein the cancer is refractory or resistant to prior treatment with an anti-PD-1 or anti-PD-L1 antibody. In some embodiments, the patient is a primary refractory patient to an anti-PD-1 or anti-PD-L1 antibody. In some embodiments, the patient shows no prior response to an anti-PD-1 or anti-PD-L1 antibody. In some embodiments, the patient shows a prior response to an anti-PD-1 or anti-PD-L1 antibody, follow by progression of the patient's cancer. In some embodiments, the cancer is refractory to an anti-CTLA-4 antibody and/or an anti-PD-1 or anti-PD-L1 antibody in combination with at least one chemotherapeutic agent. In some embodiments, the prior chemotherapeutic agent is carboplatin, paclitaxel, pemetrexed, and/or cisplatin. In some prior embodiments, the chemotherapeutic agent(s) is a platinum doublet chemotherapeutic agent. In some embodiments, the platinum doublet therapy comprises a first chemotherapeutic agent selected from the group consisting of cisplatin and carboplatin and a second chemotherapeutic agent selected from the group consisting of vinorelbine, gemcitabine and a taxane (including for example, paclitaxel, docetaxel or nab-paclitaxel). In some embodiments, the platinum doublet chemotherapeutic agent is in combination with pemetrexed.

In some embodiments, the NSCLC is PD-L1 negative and/or is from a patient with a cancer that expresses PD-L1 with a tumor proportion score (TPS) of <1%, as described elsewhere herein.

In some embodiments, the NSCLC is refractory to a combination therapy comprising an anti-PD-1 or the anti-PD-L1 antibody and a platinum doublet therapy, wherein the platinum doublet therapy comprises:
i. a first chemotherapeutic agent selected from the group consisting of cisplatin and carboplatin,
ii. and a second chemotherapeutic agent selected from the group consisting of vinorelbine, gemcitabine and a taxane (including for example, paclitaxel, docetaxel or nab-paclitaxel).

In some embodiments, the NSCLC is refractory to a combination therapy comprising an anti-PD-1 or the anti-PD-L1 antibody, pemetrexed, and a platinum doublet therapy, wherein the platinum doublet therapy comprises:
i. a first chemotherapeutic agent selected from the group consisting of cisplatin and carboplatin,
ii. and a second chemotherapeutic agent selected from the group consisting of vinorelbine, gemcitabine and a taxane (including for example, paclitaxel, docetaxel or nab-paclitaxel).

In some embodiments, the NSCLC has been treated with an anti-PD-1 antibody. In some embodiments, the NSCLC has been treated with an anti-PD-L1 antibody. In some embodiments, the NSCLC patient is treatment naïve. In some embodiments, the NSCLC has not been treated with an anti-PD-1 antibody. In some embodiments, the NSCLC has not been treated with an anti-PD-L1 antibody. In some embodiments, the NSCLC has been previously treated with a chemotherapeutic agent. In some embodiments, the NSCLC has been previously treated with a chemotherapeutic agent but is not longer being treated with the chemotherapeutic agent. In some embodiments, the NSCLC patient is anti-PD-1/PD-L1 naïve. In some embodiments, the NSCLC patient has low expression of PD-L1. In some embodiments, the NSCLC patient has treatment naïve NSCLC or is post-chemotherapeutic treatment but anti-PD-1/PD-L1 naïve. In some embodiments, the NSCLC patient is treatment naïve or post-chemotherapeutic treatment but anti-PD-1/PD-L1 naïve and has low expression of PD-L1. In some embodiments, the NSCLC patient has bulky disease at baseline. In some embodiments, the subject has bulky disease at baseline and has low expression of PD-L1. In some embodiments, the NSCLC patient has no detectable expression of PD-L1. In some embodiments, the NSCLC patient is treatment naïve or post-chemotherapeutic treatment but anti-PD-1/PD-L1 naïve and has no detectable expression of PD-L1. In some embodiments, the patient has bulky disease at baseline and has no detectable expression of PD-L1. In some embodiments, the NSCLC patient has treatment naïve NSCLC or post chemotherapy (e.g., post chemotherapeutic agent) but anti-PD-1/PD-L1 naïve who have low expression of PD-L1 and/or have bulky disease at baseline. In some embodiments, bulky disease is indicated where the maximal tumor diameter is greater than 7 cm measured in either the transverse or coronal plane. In some embodiments, bulky disease is indicated when there are swollen lymph nodes with a short-axis diameter of 20 mm or greater. In some embodiments, the chemotherapeutic includes a standard of care therapeutic for NSCLC.

In some embodiments, PD-L1 expression is determined by the tumor proportion score. In some embodiments, the subject with a refractory NSCLC tumor has a <1% tumor proportion score (TPS). In some embodiments, the subject with a refractory NSCLC tumor has a ≥10% TPS. In some embodiments, subject with the refractory NSCLC has been previously treated with an anti-PD-1 and/or anti-PD-L1 antibody and the tumor proportion score was determined prior to said anti-PD-1 and/or anti-PD-L1 antibody treatment. In some embodiments, subject with the refractory NSCLC has been previously treated with an anti-PD-L1 antibody and the tumor proportion score was determined prior to said anti-PD-L1 antibody treatment.

In some embodiments, the TILs prepared by the methods of the present invention, including those as described for example in FIG. 1, exhibit increased polyclonality as compared to TILs produced by other methods, including those not exemplified in FIG. 1, such as for example, methods referred to as process 1C methods. In some embodiments, significantly improved polyclonality and/or increased polyclonality is indicative of treatment efficacy and/or increased clinical efficacy for cancer treatment. In some embodiments, polyclonality refers to the T-cell repertoire diversity. In some embodiments, an increase in polyclonality can be indicative of treatment efficacy with regard to administration of the TILs produced by the methods of the present invention. In some embodiments, polyclonality is increased one-fold, two-fold, ten-fold, 100-fold, 500-fold, or 1000-fold as compared to TILs prepared using methods than those provide herein including for example, methods other than those embodied in FIG. 1. In some embodiments, polyclonality is increased one-fold as compared to an untreated patient and/or as compared to a patient treated with TILs prepared using other methods than those provide herein including for example, methods other than those embodied in FIG. 1. In some embodiments, polyclonality is increased two-fold as compared to an untreated patient and/or as compared to a patient treated with TILs prepared using other methods than those provide herein including for example, methods other than those embodied in FIG. 1. In some embodiments, polyclonality is increased ten-fold as compared to an untreated patient and/or as compared to a patient treated with TILs prepared using other methods than those provide herein including for example, methods other than those embodied in FIG. 1. In some embodiments, polyclonality is increased 100-fold as compared to an untreated patient and/or as compared to a patient treated with TILs prepared using other methods than those provide herein including for example, methods other than those embodied in FIG. 1. In some embodiments, polyclonality is increased 500-fold as compared to an untreated patient and/or as compared to a patient treated with TILs prepared using other methods than those provide herein including for example, methods other than those embodied in FIG. 1. In some embodiments, polyclonality is increased 1000-fold as compared to an untreated patient and/or as compared to a patient treated with TILs prepared using other methods than those provide herein including for example, methods other than those embodied in FIG. 1.

In some embodiments, PD-L1 expression is determined by the tumor proportion score using one more testing methods as described herein. In some embodiments, the subject or patient with a NSCLC tumor has a <1% tumor proportion score (TPS). In some embodiments, the NSCLC tumor has a ≥1% TPS. In some embodiments, the subject or patient with the NSCLC has been previously treated with an anti-PD-1 and/or anti-PD-L1 antibody and the tumor proportion score was determined prior to the anti-PD-1 and/or anti-PD-L1 antibody treatment. In some embodiments, the subject or patient with the NSCLC has been previously treated with an anti-PD-L1 antibody and the tumor proportion score was determined prior to the anti-PD-L1 antibody treatment. In some embodiments, the subject or patient with a refractory or resistant NSCLC tumor has a <1% tumor proportion score (TPS). In some embodiments, the subject or patient with a refractory or resistant NSCLC tumor has a ≥1% TPS. In some embodiments, the subject or patient with the refractory or resistant NSCLC has been previously treated with an anti-PD-1 and/or anti-PD-L1 antibody and the tumor proportion score was determined prior to the anti-PD-1 and/or anti-PD-L1 antibody treatment. In some embodiments, the subject or patient with the refractory or resistant NSCLC has been previously treated with an anti-PD-L1 antibody and the tumor proportion score was determined prior to the anti-PD-L1 antibody treatment.

In some embodiments, the NSCLC is an NSCLC that exhibits a tumor proportion score (TPS), or the percentage of viable tumor cells from a patient taken prior to anti-PD-1 or anti-PD-L1 therapy, showing partial or complete membrane staining at any intensity, for the PD-L1 protein that is less than 1% (TPS<1%). In an embodiment, the NSCLC is an NSCLC that exhibits a TPS selected from the group consisting of <50%, <45%, <40%, <35%, <30%, <25%, <20%, <15%, <10%, <9%, <8%, <7%, <6%, <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02%, and <0.01%. In an embodiment, the NSCLC is an NSCLC that exhibits a TPS selected from the group consisting of about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, and about 0.01%. In an embodiment, the NSCLC is an NSCLC that exhibits a TPS between 0% and 1%. In an embodiment, the NSCLC is an NSCLC that exhibits a TPS between 0% and 0.9%. In an embodiment, the NSCLC is an NSCLC that exhibits a TPS between 0% and 0.8%. In an embodiment, the NSCLC is an NSCLC that exhibits a TPS between 0% and 0.7%. In an embodiment, the NSCLC is an NSCLC that exhibits a TPS between 0% and 0.6%. In an embodiment, the NSCLC is an NSCLC that exhibits a TPS between 0% and 0.5%. In an embodiment, the NSCLC is an NSCLC that exhibits a TPS between 0% and 0.4%. In an embodiment, the NSCLC is an NSCLC that exhibits a TPS between 0% and 0.3%. In an embodiment, the NSCLC is an NSCLC that exhibits a TPS between 0% and 0.2%. In an embodiment, the NSCLC is an NSCLC that exhibits a TPS between 0% and 0.1%. TPS may be measured by methods known in the art, such as those described in Hirsch, et al. J. Thorac. Oncol. 2017, 12, 208-222 or those used for the determination of TPS prior to treatment with pembrolizumab or other anti-PD-1 or anti-PD-L1 therapies. Methods for measurement of TPS that have been approved by the U.S. Food and Drug Administration may also be used. In some embodiments, the PD-L1 is exosomal PD-L1. In some embodiments, the PD-L1 is found on circulating tumor cells.

In some embodiments, the partial membrane staining includes 1%, 5%, 10%, 15%, 20%, 25%, 30%, 35%, 40%, 45%, 50%, 55%, 60%, 65%, 70%, 75%, 80%, 85%, 90%, 95%, 97%, 99%, or more. In some embodiments, the completed membrane staining includes approximately 100% membrane staining.

In some embodiments, testing for PD-L1 can involve measuring levels of PD-L1 in patient serum. In these embodiments, measurement of PD-L1 in patient serum removes the uncertainty of tumor heterogeneity and the patient discomfort of serial biopsies.

In some embodiments, elevated soluble PD-L1 as compared to a baseline or standard level correlates with worsened prognosis in NSCLC. See, for example, Okuma, et al., Clinical Lung Cancer, 2018, 19, 410-417; Vecchiarelli, et al., Oncotarget, 2018, 9, 17554-17563. In some embodiments, the PD-L1 is exosomal PD-L1. In some embodiments, the PD-L1 is expressed on circulating tumor cells.

In an embodiment, the invention provides a method of treating non-small cell lung carcinoma (NSCLC) by administering a population of tumor infiltrating lymphocytes (TILs) to a subject or patient in need thereof, wherein the subject or patient has at least one of
i. a predetermined tumor proportion score (TPS) of PD-L1<1%,
ii. a TPS score of PD-L1 of 1%-49%, or
iii. a predetermined absence of one or more driver mutations,
   wherein the driver mutation is selected from the group consisting of an EGFR mutation, an EGFR insertion, an EGFR exon 20 mutation, a KRAS mutation, a BRAF mutation, an ALK mutation, a c-ROS mutation (ROS1 mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation, and wherein the method comprises:
(a) obtaining and/or receiving a first population of TILs from a tumor resected from the subject or patient by processing a tumor sample obtained from the subject into multiple tumor fragments;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting therapeutic population of TILs obtained from step (d), wherein the transition from step (d) to step (e) occurs without opening the system; and
(f) transferring the harvested TIL population from step (e) to an infusion bag, wherein the transfer from step (e) to (f) occurs without opening the system;
(g) cryopreserving the infusion bag comprising the harvested TIL population from step (f) using a cryopreservation process; and
(h) administering a therapeutically effective dosage of the third population of TILs from the infusion bag in step (g) to the subject or patient.

In an embodiment, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, an EGFR exon 20 mutation, a KRAS mutation, a BRAF mutation, an ALK mutation, a c-ROS mutation (ROS1 mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation,
(c) determining that the patient has a TPS score for PD-L1 of about 1% to about 49% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In an embodiment, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, an EGFR exon 20 mutation, a KRAS mutation, a BRAF mutation, an ALK mutation, a c-ROS mutation (ROS1 mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation,
(c) determining that the patient has a TPS score for PD-L1 of less than about 1% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein he first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In an embodiment, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, a KRAS mutation, a BRAF mutation, an ALK mutation, a c-ROS mutation (ROS1 mutation), a ROS1 fusion, a RET mutation, or a RET fusion,
(c) determining that the patient has a TPS score for PD-L1 of about 1% to about 49% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In an embodiment, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, a KRAS mutation, a BRAF mutation, an ALK mutation, a c-ROS mutation (ROS1 mutation), a ROS1 fusion, a RET mutation, or a RET fusion,
(c) determining that the patient has a TPS score for PD-L1 of less than about 1% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In another embodiment, the invention provides a method for treating a subject with cancer comprising administering to the subject a therapeutically effective dosage of the therapeutic TIL population described herein.

In another embodiment, the invention provides a method for treating a subject with cancer comprising administering to the subject a therapeutically effective dosage of the TIL composition described herein.

In another embodiment, the invention provides a method for treating a subject with cancer described herein modified such that prior to administering the therapeutically effective dosage of the therapeutic TIL population and the TIL composition described herein, respectively, a non-myeloablative lymphodepletion regimen is administered to the subject. Suitable non-myeloablative lymphodepletion regimens are described herein.

In another embodiment, the invention provides a method for treating a subject with cancer described herein modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In an embodiment, the invention provides a method for treating a subject with a cancer as described herein using TILs, MILs, or PBLs as described herein, optionally genetically modified to express a CCR and/or a chemokine receptor as described herein, the method further including the step of administering a lymphodepletion regimen comprising apamistamab-¹³¹I, or variants, fragments, or biosimilars thereof. Apamistamab-¹³¹I, also known as IOMAB-ACT, is an anti-CD45 antibody available from Actinium Pharmaceuticals, Inc.

In an embodiment, the invention provides a method for treating a subject with a cancer as described herein using TILs, MILs, or PBLs as described herein, optionally genetically modified to express a CCR and/or a chemokine receptor as described herein, the method further including the step of administering a lymphodepletion regimen comprising alemtuzumab, or variants, fragments, or biosimilars thereof. Alemtuzumab, also known as LEMTRADA, is available from Sanofi, Inc.

In an embodiment, the invention provides a method for treating a subject with a cancer as described herein using TILs, MILs, or PBLs as described herein, wherein the use of a CCR as described herein replaces the step the subject with an IL-2 regimen, such that no IL-2 regimen is administered to the subject in conjunction with TIL, MIL, or PBL therapy. In an embodiment, the invention provides a method for treating a subject with a cancer as described herein using TILs, MILs, or PBLs as described herein, wherein no IL-2 regimen is administered to the subject in conjunction with TIL, MIL, or PBL therapy. In an embodiment, the invention provides a method for treating a subject with a cancer as described herein using TILs, MILs, or PBLs as described herein, wherein the TILs, MILs, or PBLs are modified to express a CCR, and wherein no IL-2 regimen is administered to the subject in conjunction with TIL, MIL, or PBL therapy. In an embodiment, the invention provides a method for treating a subject with a cancer as described herein using TILs, MILs, or PBLs as described herein, wherein the use of a CCR and/or a chemokine receptor as described herein with an IL-2R intracellular domain (including IL-2Rβ and IL-2Rγ domains) is used, wherein no IL-2 regimen is administered to the subject in conjunction with TIL, MIL, or PBL therapy.

In another embodiment, the invention provides a method for treating a subject with cancer described herein modified to further comprise the step of treating the subject with a high-dose IL-2 regimen starting on the day after administration of the TIL cells to the subject.

In another embodiment, the invention provides the method for treating a subject with cancer described herein modified such that the high-dose IL-2 regimen comprises 600,000 or 720,000 IU/kg administered as a 15-minute bolus intravenous infusion every eight hours until tolerance.

In another embodiment, the invention provides a method for treating a subject with cancer wherein the cancer is a solid tumor.

In another embodiment, the invention provides a method for treating a subject with cancer wherein the cancer is melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In another embodiment, the invention provides a method for treating a subject with cancer wherein the cancer is melanoma, HNSCC, cervical cancers, NSCLC, glioblastoma (including GBM), and gastrointestinal cancer.

In another embodiment, the invention provides a method for treating a subject with cancer wherein the cancer is melanoma.

In another embodiment, the invention provides a method for treating a subject with cancer wherein the cancer is HNSCC.

In another embodiment, the invention provides a method for treating a subject with cancer wherein the cancer is a cervical cancer.

In another embodiment, the invention provides a method for treating a subject with cancer wherein the cancer is NSCLC.

In another embodiment, the invention provides a method for treating a subject with cancer wherein the cancer is glioblastoma (including GBM).

In another embodiment, the invention provides a method for treating a subject with cancer wherein the cancer is gastrointestinal cancer.

In another embodiment, the invention provides a method for treating a subject with cancer wherein the cancer is a hypermutated cancer.

In another embodiment, the invention provides a method for treating a subject with cancer wherein the cancer is a pediatric hypermutated cancer.

In another embodiment, the invention provides a therapeutic TIL population described herein for use in a method for treating a subject with cancer comprising administering to the subject a therapeutically effective dosage of the therapeutic TIL population.

In another embodiment, the invention provides a TIL composition described herein for use in a method for treating a subject with cancer comprising administering to the subject a therapeutically effective dosage of the TIL composition.

In another embodiment, the invention provides a therapeutic TIL population described herein or the TIL composition described herein modified such that prior to administering to the subject the therapeutically effective dosage of the therapeutic TIL population described herein or the TIL composition described herein, a non-myeloablative lymphodepletion regimen has been administered to the subject.

In another embodiment, the invention provides a therapeutic TIL population or the TIL composition described herein modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In another embodiment, the invention provides a therapeutic TIL population or a TIL composition described herein modified to further comprise the step of treating patient with a high-dose IL-2 regimen starting on the day after administration of the TIL cells to the patient.

In another embodiment, the invention provides a therapeutic TIL population or a TIL composition described herein modified such that the high-dose IL-2 regimen comprises 600,000 or 720,000 IU/kg administered as a 15-minute bolus intravenous infusion every eight hours until tolerance.

In another embodiment, the invention provides a therapeutic TIL population or a TIL composition described herein modified such that the cancer is a solid tumor.

In another embodiment, the invention provides a therapeutic TIL population or a TIL composition for the treatment of a cancer wherein the cancer is melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In another embodiment, the invention provides a therapeutic TIL population or a TIL composition for the treatment of a cancer wherein the cancer is melanoma, HNSCC, cervical cancers, NSCLC, glioblastoma (including GBM), and gastrointestinal cancer.

In another embodiment, the invention provides a therapeutic TIL population or a TIL composition for the treatment of a cancer wherein the cancer is melanoma.

In another embodiment, the invention provides a therapeutic TIL population or a TIL composition for the treatment of a cancer wherein the cancer is HNSCC.

In another embodiment, the invention provides a therapeutic TIL population or a TIL composition for the treatment of a cancer wherein the cancer is cervical cancer.

In another embodiment, the invention provides a therapeutic TIL population or a TIL composition for the treatment of a cancer wherein the cancer is NSCLC.

In another embodiment, the invention provides a therapeutic TIL population or a TIL composition for the treatment of a cancer wherein the cancer is glioblastoma.

In another embodiment, the invention provides a therapeutic TIL population or a TIL composition for the treatment of a cancer wherein the cancer is gastrointestinal cancer.

In another embodiment, the invention provides a therapeutic TIL population or a TIL composition for the treatment of a cancer wherein the cancer is a hypermutated cancer.

In another embodiment, the invention provides a therapeutic TIL population or a TIL composition for the treatment of a cancer wherein the cancer is a pediatric hypermutated cancer.

In some embodiments, the cancer is a hypermutated cancer or hypermutated cancer phenotype. Hypermutated cancers are extensively described in Campbell, et al., Cell 2017, 171, 1042-1056; incorporated by reference herein in its entirety for all purposes). In some embodiments, a hypermutated tumors comprise between 9 and 10 mutations per megabase (Mb). In some embodiments, pediatric hypermutated tumors comprise 9.91 mutations per megabase (Mb). In some embodiments, adult hypermutated tumors comprise 9 mutations per megabase (Mb). In some embodiments, enhanced hypermutated tumors comprise between 10 and 100 mutations per megabase (Mb). In some embodiments, enhanced pediatric hypermutated tumors comprise between 10 and 100 mutations per megabase (Mb). In some embodiments, enhanced adult hypermutated tumors comprise between 10 and 100 mutations per megabase (Mb). In some embodiments, an ultra-hypermutated tumors comprise greater than 100 mutations per megabase (Mb). In some embodiments, pediatric ultra-hypermutated tumors comprise greater than 100 mutations per megabase (Mb). In some embodiments, adult ultra-hypermutated tumors comprise greater than 100 mutations per megabase (Mb).

In some embodiments, the hypermutated tumors have mutations in replication repair pathways. In some embodiments, the hypermutated tumors have mutations in replication repair associated DNA polymerases. In some embodiments, the hypermutated tumors have microsatellite instability. In some embodiments, the ultra-hypermutated tumors have mutations in replication repair associated DNA polymerases and have microsatellite instability. In some embodiments, hypermutation in the tumor is correlated with response to immune checkpoint inhibitors. In some embodiments, hypermutated tumors are resistant to treatment with immune checkpoint inhibitors. In some embodiments, hypermutated tumors can be treated using the TILs of the present invention. In some embodiments, hypermutation in the tumor is caused by environmental factors (extrinsic exposures). For example, UV light can be the primary cause of the high numbers of mutations in malignant melanoma (see, for example, Pfeifer, et al. Mutat. Res. 2005, 571, 19-31; Sage, Photochem. Photobiol. 1993, 57, 163-174). In some embodiments, hypermutation in the tumor can be caused by the greater than 60 carcinogens in tobacco smoke for tumors of the lung and larynx, as well as other tumors, due to direct mutagen exposure (see, for example, Pleasance, et al., Nature 2010, 463, 184-190). In some embodiments, hypermutation in the tumor is caused by dysregulation of apolipoprotein B mRNA editing enzyme, catalytic polypeptide-like (APOBEC) family members, which has been shown to result in increased levels of C to T transitions in a wide range of cancers (see, for example, Roberts, et al., Nat. Genet. 2013, 45, 970-976). In some embodiments, hypermutation in the tumor is caused by defective DNA replication repair by mutations that compromise proofreading, which is performed by the major replicative enzymes Pol3 and Pold1. In some embodiments, hypermutation in the tumor is caused by defects in DNA mismatch repair, which are associated with hypermutation in colorectal, endometrial, and other cancers (see, for example, Kandoth, et al., Nature 2013, 497, 67-73.; Muzny, et al., Nature 2012, 487, 330-337). In some embodiments, DNA replication repair mutations are also found in cancer predisposition syndromes, such as constitutional or biallelic mismatch repair deficiency (CMMRD), Lynch syndrome, and polymerase proofreading-associated polyposis (PPAP).

In an embodiment, the invention includes a method of treating a cancer with a population of TILs, wherein the cancer is a hypermutated cancer. In an embodiment, the invention includes a method of treating a cancer with a population of TILs, wherein the cancer is an enhanced hypermutated cancer. In an embodiment, the invention includes a method of treating a cancer with a population of TILs, wherein the cancer is an ultra-hypermutated cancer.

In another embodiment, the invention provides the use of a therapeutic TIL population described herein in a method of treating cancer in a subject comprising administering to the subject a therapeutically effective dosage of the therapeutic TIL population.

In another embodiment, the invention provides the use of a TIL composition described in any of the preceding paragraphs in a method of treating cancer in a subject comprising administering to the subject a therapeutically effective dosage of the TIL composition.

In another embodiment, the invention provides the use of a therapeutic TIL population described herein or a TIL composition described herein in a method of treating cancer in a patient comprising administering to the patient a non-myeloablative lymphodepletion regimen and then administering to the subject the therapeutically effective dosage of the therapeutic TIL population described in any of the preceding paragraphs or the therapeutically effective dosage of the TIL composition described herein.

### 1. Methods of Treating Cancers Based on Driver Mutations

As used herein, the phrases “driver mutation” and/or “actionable mutation” and/or “oncogenic driver mutation” refer to mutations that are typically considered oncogenic drivers (i.e., cancer drivers or cancer inducers). The presence of one or more of these mutations has traditionally been the utilized as the target for a targeted therapy. Often, driver mutations are examined and/or analyzed for treatment with targeted therapeutic moieties, including for example tyrosine kinase inhibitors (TKIs). Such driver mutations can, in some embodiments, impact or affect response to a first line therapeutic treatment. TIL therapy methods and compositions described herein are effective for treatment whether such driver mutations are present or absent in the patient or subject. Such driver mutations can be tested and determined by any method known in the art, including whole exome sequencing or methods targeted to the detection of a specific driver mutation.

In some embodiments, the cancer is a cancer that exhibits the presence or absence of one or more driver mutations. In some embodiments, the cancer exhibits the presence of one or more driver mutations. In some embodiments, a cancer exhibits the absence of one or more driver mutations. In some embodiments, a cancer has been analyzed for the absence or presence of one or more driver mutations. In some embodiments, the one or more driver mutations are not present. In some embodiments, a cancer treatment is independent of the presence or absence of one or more driver mutations. In some embodiments, the cancer exhibits one or more driver mutations selected from the group consisting of an EGFR mutation, an EGFR insertion, EGFR exon20, a KRAS mutation, a BRAF-mutation, a BRAF V600E mutation, a BRAF V600K mutation, a BRAF V600 mutation, an ALK mutation, a c-ROS mutation (ROS1-mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation. In some embodiments, the cancer exhibits a PD-L1 TPS of <1% and has a predetermined absence of one or more driver mutations.

In some embodiments, a cancer is an cancer that is not indicated for treatment by an EGFR inhibitor, a BRAF inhibitor, an ALK inhibitor, a c-Ros inhibitor, a RET inhibitor, an ERBB2 inhibitor, BRCA inhibitor, a MAP2K1 inhibitor, PIK3CA inhibitor, CDKN2A inhibitor, a PTEN inhibitor, an UMD inhibitor, an NRAS inhibitor, a KRAS inhibitor, an NF1 inhibitor, MET inhibitor a TP53 inhibitor, a CREBBP inhibitor, a KMT2C inhibitor, a KMT2D mutation, an ARID1A mutation, a RB1 inhibitor, an ATM inhibitor, a SETD2 inhibitor, a FLT3 inhibitor, a PTPN11 inhibitor, a FGFR1 inhibitor, an EP300 inhibitor, a MYC inhibitor, an EZH2 inhibitor, a JAK2 inhibitor, a FBXW7 inhibitor, a CCND3 inhibitor, and a GNAT 1 inhibitor.

In some embodiments, a cancer exhibits a PD-L1 TPS of <1% and is not indicated for treatment by an EGFR inhibitor, a BRAF inhibitor, an ALK inhibitor, a c-Ros inhibitor, a RET inhibitor, an ERBB2 inhibitor, BRCA inhibitor, a MAP2K1 inhibitor, PIK3CA inhibitor, CDKN2A inhibitor, a PTEN inhibitor, an UMD inhibitor, an NRAS inhibitor, a KRAS inhibitor, an NF1 inhibitor, MET inhibitor a TP53 inhibitor, a CREBBP inhibitor, a KMT2C inhibitor, a KMT2D mutation, an ARID1A mutation, a RB1 inhibitor, an ATM inhibitor, a SETD2 inhibitor, a FLT3 inhibitor, a PTPN11 inhibitor, a FGFR1 inhibitor, an EP300 inhibitor, a MYC inhibitor, an EZH2 inhibitor, a JAK2 inhibitor, a FBXW7 inhibitor, a CCND3 inhibitor, and a GNA11 inhibitor

In some embodiments, the cancer is NSCLC, and an EGFR mutation results in tumor transformation from NSCLC to small cell lung cancer (SCLC).

In some embodiments, a cancer (or a biopsy thereof) exhibits high-tumor mutational burden (high-TMB; >10 mut/kb) and/or microsatellite instability high (MSI-high). In some embodiments, the cancer (or a biopsy thereof) exhibits high-tumor mutational burden (high-TMB; >10 mut/kb). In some embodiments, a cancer (or a biopsy thereof) exhibits microsatellite instability high (MSI-high). Methods and systems for evaluating tumor mutational burden are known in the art. Exemplary disclosures of such methods and systems can be found in U.S. Pat. No. 9,792,403, U.S. Patent Application Publication No. US 2018/0363066 A1, International Patent Application Publication Nos. WO 2013/070634 A1 and WO 2018/106884 A1, and Metzker, Nature Biotechnol. Rev. 2010, 11, 31-46, each of which is incorporated by reference herein.

In some embodiments, an EGFR mutation includes, for example, but is not limited to T790M, Ex19Del, L858R, Exon 20 insertion, delE709-T710insD, 1744_K745insKIPVAI, K745_E746insTPVAIK, E709X, E709K, E709A, Exon 18 deletion, G719X, G719A, G719S, L861Q, S768I, L747P, A763_764insFQEA, D770_N771insNPG, A763_764insFQEA, P772_H773insDNP exon 20 insertion, H773_V774insNPH exon 20 insertion, S768I, D770_N771insSVD, V769_D770InsASV, p.K745_E746insIPVAIK, p.K745_E746insTPVAIK, p.I744_K745insKIPVAI, D770_N771insNPG, P772_H773insPNP, A763_Y764insFQEA, and/or EGFR kinase domain duplication (EGFR-KDD). In some embodiments, an EGFR mutation is selected from the group consisting of T790M, Ex19Del, L858R, Exon 20 insertion, delE709-T710insD, 1744_K745insKIPVAI, K745_E746insTPVAIK, E709X, E709K, E709A, Exon 18 deletion, G719X, G719A, G719S, L861Q, S768I, L747P, A763_764insFQEA, D770_N771insNPG, A763_764insFQEA, P772_H773insDNP exon 20 insertion, H773_V774insNPH exon 20 insertion, S768I, D770_N771insSVD, V769_D770InsASV, p.K745_E746insIPVAIK, p.K745_E746insTPVAIK, p.I744_K745insKIPVAI, D770_N771insNPG, P772_H773insPNP, A763_Y764insFQEA, and EGFR kinase domain duplication (EGFR-KDD).

In some embodiments, an EGFR mutation is a double mutation, including, but not limited to, L858R/T790M, Ex19Del/T790M, G719X/L861Q, G719X/S768I (or S768I/G719X), S768I/L858R, L858R/E709A, and/or E746_T751delinsA+T790M. In some embodiments, an EGFR mutation is a double mutation selected from group consisting of L858R/T790M, Ex19Del/T790M, G719X/L861Q, G719X/S768I (or S768I/G719X), S768I/L858R, L858R/E709A, and E746_T751delinsA+T790M. Additional properties and methods regarding EGFR mutations are provided in International Patent Application Publication No. WO 2010/020618 A1, which is incorporated by referenced herein.

In some embodiments, the ALK mutation includes, but not limited to, EML4-ALK Variant 1 (AB274722.1; BAF73611.1), EML4-ALK Variant 2 (AB275889.1; BAF73612.1), EML4-ALK Variant 3a (AB374361.1; BAG55003.1), EML4-ALK Variant 3b (AB374362.1; BAG55004.1), EML4-ALK Variant 4 (AB374363.1; BAG75147.1), EML4-ALK Variant 5a (AB374364.1; BAG75148.1), EML4-ALK Variant 5b (AB374365.1; BAG75149.1), EML4-ALK Variant 6 (AB462411.1; BAH57335.1), EML4-ALK Variant 7 (AB462412.1; BAH57336.1), KIF5B-ALK (AB462413.1; BAH57337.1), NPM-ALK, TPM3-ALK, TFGXL-ALK, TEGL-ALK, TFGS-ALK, A11C-ALK, CLTC-ALK, MSN-ALK, TPM4-ALK, MYH9-ALK, RANBP2-ALK, AL017-ALK, and CARS-ALK (see, for example. Pulford et al., (2004) J. Cell. Physiol. 199:330-358). In addition, a skilled artisan will understand that ALK kinase variants can arise depending upon the particular fusion event between an ALK kinase and its fusion partner (e.g., EML4 can fuse at least exon 2, 6a, 6b, 13, 14, and/or 15, as described, for example, in Horn and Pao, J. Clin. Oncol. 2009, 27, 4247-4253, the disclosure of which is incorporated by reference herein.

Additional examples of ALK mutations are described in U.S. Pat. Nos. 9,018,230 and 9,458,508, the disclosures of which are incorporated by reference herein.

In some embodiments, the ROS1 mutation of the present invention is a ROS1 fusion, where a portion of the ROS1 polypeptide that includes the kinase domain of the ROS1 protein (or polynucleotide encoding the same) fused to all or a portion of another polypeptide (or polynucleotide encoding the same) and where the name of that second polypeptide or polynucleotide is named in the fusion. In some embodiments, the ROS1 mutation is determined as ROS1-fusion protein (e.g., by IHC) and/or ROS-fusion gene (e.g. by FISH), and/or ROS1 mRNA (e.g. by qRT-PCR), preferably indicative of a ROS1-fusion protein selected from the group consisting of SLC34A2-ROS1 (SLC34A2 exons 13del2046 and 4 fused to ROS1 exons 32 and 34), CD74-ROS1 (CD74 exon 6 fused to ROS1 exons 32 and 34), EZR-ROS1 (EZR exon 10 fused to ROS1 exon 34), TPM3-ROS1 (TPM3 exon 8 fused to ROS1 exon 35), LRIG3-ROS1 (LRIG3 exon 16 fused to ROS1 exon 35), SDC4-ROS1 (SDC4 exon 2 and 4 fused to ROS1 exon 32 and SDC4 exon 4 fused to ROS1 exon 34), GOPC-ROS1, also known as FIG-ROS1, (GOPC exon 8 fused to ROS1 exon 35 and GOPC exon 4 fused to ROS1 exon 36), and G2032R, also known as ROS1^{G2032R}.

Additional disclosures of ROS1 mutations and a ROS fusion have been provided in U.S. Patent Application Publication Nos. US 2010/0221737 A1, US 2015/0056193 A1, and US 2010/0143918 A1, and in International Patent Application Publication No WO 2010/093928 A1, each of which are incorporated by reference herein. In some embodiments, the RET mutation is a RET fusion or point mutation.

In some embodiments, a RET point mutation includes but is not limited to H6650, K666E, K666M, S686N, G691S, R694Q, M700L, V706M, V706A, E713K, G736R, G748C, A750P, S765P, P766S, E768Q, E768D, L769L, R770Q, D771N, N777S, V7781, Q781R, L790F, Y791F, Y791N, V804L, V804M, V804E, E805K, E806C, Y806E, Y806F, Y806S, Y806G, Y806C, E818K, S8191, G823E, Y826M, R833C, P841L, P841P, E843D, R844W, R844Q, R844L, M848T, 1852M A866W, R873W, A876V, L881V, A883F, A883S, A883T, E884K, R886W, S891A, R8970, D898V, E901K, 5904F, S904C2, K907E, K907M, R908K, G911D, R912P, R912Q M918T, M918V, M918L6, A919V, E921K, S922P, S922Y, T930M, F961L, R972G, R982C, M1009V, D1017N, V10416, and M1064T.

In some embodiments, a RET fusion is a fusion between RET and a fusion partner that is selected from the group consisting of BCR, BCR, CLIP 1, KIFSB, CCDC6, PTClex9, NCOA4, TRIM33, ERC1, FGFRIOP, MBD1, RAB61P2, PRKARIA, TRIM24, KTN1, GOLGA5, HOOK3, KIAA1468, TRIM27, AKAP13, FKBP15, SPECCIL, TBL1XR1, CEP55, CUX1, ACBD5, MYH13, PIBFT, KIAA1217, and MPRIP.

Additional disclosures of a RET mutations has been provided in U.S. patent Ser. No. 10/035,789, which is hereby incorporated by reference in their entirety.

In some embodiments, a BRAF mutation is BRAF V600E/K mutation. In other embodiments, the BRAF mutation is a non-V600E/K mutation.

In some embodiments, a non-V600E/K BRAF mutation is a kinase-activated mutation, a kinase-impaired mutation, or a kinase-unknown mutation, and combinations thereof. In some embodiments, a kinase-activated mutation is selected from the group consisting of R4621, 1463S, G464E, G464R, G464V, G466A, G469A, N58 is, E586K, F595L, L597Q, L597R, L5975, L597V, A598V, T599E, V600R, K601E, 5602D, A728V, and combinations thereof. In some embodiments, a kinase-impaired mutation is selected from the group consisting of G466E, G466R, G466V, Y472C, K483M, D594A, D594E, D594G, D594H, D594N, D594V, G596R, T599A, 5602A, and combinations thereof. In some embodiments, a kinase-unknown mutation is selected from the group consisting of T4401, 5467L, G469E, G469R, G4695, G469V, L584F, L588F, V600 K6OldelinsE, 56051, Q609L, E611Q, and combinations thereof. In some embodiments, the non-V600E/K BRAF mutation is selected from the group consisting of D594, G469, K601E, L597, T599 duplication, L485W, F247L, G466V, BRAF fusion, BRAF-AGAP3 rearrangement, BRAF exon 15 slice variant, and combinations thereof.

In some embodiments, a Met mutation includes point mutation, deletion mutation, insertion mutation, inversion, aberrant splicing, missense mutation, or gene magnification that causes the increase of at least one bioactivity of c-Met protein, the tyrosine kinase activity such as improved, receptor homolog dimerization Ligand binding of formation, enhancing of body and heterodimer etc. The Met mutation can be located at any part of c-Met genes. In one embodiment, the mutation is in the kinase domain of c-Met protein encoded by the c-MET gene. In some embodiments, the c-Met mutations are point mutation at N375, V13, V923, R175, V136, L229, S323, R988, S1058/T1010 and E168.

In some embodiments, an ERBB2 mutation is a point mutation in the amino acid sequence of ERBB2. In some embodiments, the point mutation of ERBB2 is one that causes amino acid substitutions, causes mRNA splicing, or is a point mutation in the upstream region. Wherein the mutation comprises a nucleotide mutation causing at least one amino acid substitution selected from the group consisting of Q568E, P601R, I628M, P885S, R143Q, R434Q, and E874K.

In some embodiments, an ERBB2 mutation is ERBB2 amplification. In some embodiments, the ERBB2 amplification includes point mutation selected from the group consisting of V659E, G309A, G309E, S310F, D769H, D769Y, V777L, P780ins, P780-Y781insGSP, V842I, R896C, K753E, and L755S and can be detected by polymerase chain reaction or other sequencing techniques known in the art, such as those described in Bose, et al., Cancer Discov. 2013, 3(2), 224-237; and Zuo, et al. Clin Cancer Res. 2016, 22(19), 4859-4869, the disclosures of which are incorporated by reference herein.

In some embodiments, a BRCA mutation is a mutation in BRCA1 and/or BRCA2, preferably BRCA1, and/or in one or more other genes of which the protein product associates with BRCA1 and/or BRCA2 at DNA damage sites, including ATM, ATR, Chk2, H2AX, 53BP1, NFBD1, Mre11, Rad50, Nibrin, BRCA1-associated RING domain (BARD1), Abraxas, and MSH2. A mutation in one or more of these genes may result in a gene expression pattern that mimics a mutation in BRCA1 and/or BRCA2.

In certain embodiments, a BRCA mutation comprises a non-synonymous mutation. In some embodiments, a BRCA mutation comprises a nonsense mutation. In some embodiments, the BRCA mutation comprises a frameshift mutation. In some embodiments, the BRCA mutation comprises a splicing mutation. In some embodiments, a BRCA mutation is expressed as a mutant mRNA and ultimately a mutant protein. In some embodiments, a BRCA1/2 protein is functional. In other embodiments, a BRCA1/2 protein has reduced activity. In other embodiments, a BRCA1/2 protein is non-functional.

As used herein with regard to substitutions, the “=” sign with regard to mutations generally refers to synonymous substitutions, silent codons, and/or silent substitutions. In particular, a synonymous substitution (also called a silent substitution or silent codon) refers to the substitution of one nucleotide base for another in an exon of a gene encoding a protein, wherein the produced amino acid sequence is not modified. This is due to the fact that the genetic code is “degenerate”, i.e., that some amino acids are coded for by more than one three-base-pair codon. Because some of the codons for a given amino acid vary by just one base pair from others coding for the same amino acid, a point mutation that replaces the wild-type base by one of the alternatives will result in incorporation of the same amino acid into the elongating polypeptide chain during translation of the gene. In some embodiments, synonymous substitutions and mutations affecting noncoding DNA are often considered silent mutations; however, it is not always the case that the mutation is silent and without any impact. For example, a synonymous mutation can affect transcription, splicing, mRNA transport, and translation, any of which could alter the resulting phenotype, rendering the synonymous mutation non-silent. The substrate specificity of the tRNA to the rare codon can affect the timing of translation, and in turn the co-translational folding of the protein. This is manifested in the codon usage bias that has been observed in many species. A nonsynonymous substitution/mutation results in a change in amino acid that may be arbitrarily classified as conservative (a change to an amino acid with similar physiochemical properties), semi-conservative (e.g. negatively to positively charged amino acid), or radical (vastly different amino acid). In some embodiments, the BRCA mutation is a BRCA1 mutation that includes, but is not limited to P871L, K1183R, D693N, S1634G, E1038G, S1040N, S694= (=: silence codon), M16731, Q356R, S1436=, L771=, K654Sfs*47, S198N, R496H, R841W, R1347G, H619N, S1533I, L30=, A622V, Y655Vfs*18, R496C, E597K, R1443*, E23Vfs*17, L30F, E111Gfs*3, K339Rfs*2, L512F, D693N, P871S, S1140G, Q1240*, P1770S, R7=, L52F, T176M, A224S, L347=, S561F, E597*, K820E, K893Rfs*107, E962K, M1014I, R1028H, E1258D, E1346K, R1347T, L1439F, H1472R, Q1488*, S1572C, E1602K, R1610C, L1621=, Q1625*, Q1625=, D1754N, R1772Q, R1856*, and any combination thereof.

In some embodiments, a BRCA mutation is a BRCA2 mutation that includes, but is not limited to V2466A, N289H, N991D, S455= (=: silence codon), N372H, H743=, V1269=, S2414=, V2171=, L1521=, T3033Nfs*11, K1132=, T3033Lfs*29, R2842C, N1784Tfs*7, K3326*, K3326*, D1420Y, 1605Yfs*9, I3412V, A2951T, T3085Nfs*26, R2645Nfs*3, S1013*, T1915M, F3090=, V3244I, A1393V, R2034C, L1356=, E2981Rfs*37, N1784Kfs*3, K3416Nfs*11, K1691Nfs*15, S1982Rfs*22, and any combination thereof.

In some embodiments, an NRAS mutation includes but is not limited to E63K, Q61R, Q61K, G12D, G13D, Q61R, Q61L, Q61K, G12S, G12C, G13R, Q61H, G12V, G12A, Q61L, G13V, Q61H, Q61H, G12R, G13C, Q61P, G13S, G12D, G13A, G13D, A18T, Q61X, G60E, G12S, Q61= (=: silence codon), Q61E, Q61R, A146T, A59T, A59D, Q61=, R68T, A146T, G12A, E62Q, G75=, A91V, and any combination thereof.

E132KIn some embodiments, a PIK3CA mutation includes substitution mutations, deletion mutations, and insertion mutations. In some embodiments, mutations occur in PIK3CA's helical domain and in its kinase. In other embodiments, in PIK3CA's P85BD domain. In some embodiments, the PIK3CA mutation is in exon 1, 2, 4, 5, 7, 9, 13, 18, and 20. In some embodiments, the PIK3CA mutation is in exons 9 and 20. In yet other embodiments, the PIK3CA mutation is a combination of the any mutations listed above. Any combination of these exons can be tested, optionally in conjunction with testing other exons. Testing for mutations can be done along the whole coding sequence or can be focused in the areas where mutations have been found to cluster. Particular hotspots of mutations occur at nucleotide positions 1624, 1633, 1636, and 3140 of a PIK3CA coding sequence.

In some embodiments, the size of a PIK3CA mutation is small, ranging from 1 to 3 nucleotides. In some embodiments, the PIK3CA mutations include, but are not limited to G1624A, G1633A, C1636A, A3140G, G113A, T1258C, G3129T, C3139T, E542K, E545K, Q546R, H1047L, H1047R and G2702T.

In some embodiments, a MAP2K1 mutation is a somatic MAP2K1 mutation, optionally a MAP2K1 mutation that upregulates MEK1 levels. In some embodiments, the MAP2K1 mutation is a mutation in one or more genes associated with the RAS/MAPK pathway, comprising: HRAS, KRAS, NRAS, ARAF, BRAF, RAF1, MAP2K2, MAPK1, MAPK3, MAP3K3. In certain embodiments, the MAP2K1 mutation is in one or more genes selected from the group consisting of RASA, PTEN, ENG, ACVRL1, SMAD4, GDF2 or combinations thereof.

In some embodiments, a MAP2K1 mutation includes, but is not limited to, P124S, Q56P, K57N, E203K, G237*, P124L, G128D, D67N, K57E, E102_I103del, C121S, K57T, K57N, Q56P, P124L, K57N, G128V, Q58_E62del, F53L, I126=, I103_K104del, and any combination thereof.

In some embodiments, a KRAS mutation comprises anon-synonymous mutation. In some embodiments, a KRAS mutation comprises a nonsense mutation. In some embodiments, a KRAS mutation comprises a frameshift mutation. In some embodiments, a KRAS mutation comprises a splicing mutation. In some embodiments, a KRAS mutation is expressed as a mutant mRNA and ultimately a mutant protein. In some embodiments, a mutated KRAS protein is functional. In other embodiments, a mutated KRAS protein has reduced activity. In other embodiments, a mutated KRAS protein is non-functional.

In some embodiments, a KRAS mutation includes but is not limited to G12D, G12V, G13D, G12C, G12A, G12S, G12R, G13C, Q61H, A146T, Q61R, Q61H, Q61L, G13S, A146V, Q61K, G13R, G12F, K117N, G13A, G13V, A59T, V14I, K117N, Q22K, Q61P, A146P, G13D, L19F, L19F, Q61K, G12V, G60=, G12=, G13=, A18D, T58I, Q61E, E63K, G12L, GT3V, A59G, G60D, G10R, G10dup, D57N, A59E, V14G, D33E, G12I, G13dup, and any combination thereof, wherein = is indicative of silence coding.

In some embodiments, a NF1 mutation includes substitution mutations, deletion mutations, missense mutations, aberrant splicing mutations, and insertion mutations. In some embodiments, the NF1 mutation is a loss of function (LOF) mutation. In some embodiments, the NF1 mutation is selected from the group consisting of R1947X (C5839T), R304X, exon 37 mutation, exon 4b mutation, exon 7 mutation, exon 10b mutation, and exon 10c mutation (e.g., 1570G→T, E524X).

In some embodiments, a CDKN2A mutation includes but is not limited to R24P, D108G, D108N, D108Y, G125R, P114L, R80*, R58*, H83Y, W110*, P114L, E88*, W110*, E120*, D108Y, D84Y, D84N, E69*, P81L, Q50*, L78Hfs*41, D108N, S12*, P48L, E61*, Y44*, E88K, R80*, D84G, L16Pfs*9, Y129*, D108H, A148T, A36G, A102V, W15*, H83R, A57V, E33*, D74Y, A76V, E153K, D74N, H83D, V82M, R58*, Y129*, E119*, Y44*, D74A, T18_A19dup, Y44Lfs*76, L32_L37del, V28_E33del, D14_L16del, A68T, or any combination thereof.

In some embodiments, a PTEN mutation comprises a non-synonymous mutation. In some embodiments, the PTEN mutation comprises a nonsense mutation. In some embodiments, the PTEN mutation comprises a frameshift mutation. In some embodiments, the PTEN mutation comprises a splicing mutation. In some embodiments, the mutated PTEN is expressed as an mRNA and ultimately a protein. In some embodiments, the mutated PTEN protein is functional. In other embodiments, the mutated PTEN protein has reduced activity. In other embodiments, the mutated PTEN protein is non-functional. In some embodiments, the PTEN mutation includes, but is not limited to, R130Q, R130G, T319*, R233*, R130*, K267Rfs*9, N323Mfs*21, N323Kfs*2, R173C, R173H, R335*, Q171*, Q245*, E7*, D268Gfs*30, R130Q, Q214*, R130L, C136R, Q298*, Q17*, H93R, P248Tfs*5, I33del, R233*, E299*, G132D, Y68H, T319Kfs*24, N329Kfs*14, V166Sfs*14, V290*, T319Nfs*6, R142W, P38S, A126T, H61R, F278L, S229*, R130P, G129R, R130Qfs*4, P246L, R130*, G165R, C136Y, R173C, I101T, Y155C, D92E, K164Rfs*3, N184Efs*6, G129E, R130G, G36R, F341V, H123Y, C124S, M35VG127E, G165E and any combination thereof.

In some embodiments, a TP53 mutation includes, but is not limited to, R175H, G245S, R248Q, R248W, R249S, R273C, R273H, R282W, C135Y, C141Y, P151S, V157F, R158L, Y163C, V173L, V173M, C176F, H179R, H179Y, H179Q, Y205C, Y220C, Y234C, M2371, C238Y, S241F, G245D, G245C, R248L, R249M, V272M, R273L, P278L, R280T, E285K, E286K, R158H, C176Y, I195T, G214R, G245V, G266R, G266E, P278S, R280K, or any combination thereof. In some further embodiments, the TP53 mutation is selected from the group consisting of: G245S; R249S; R273C; R273H; C141Y, V157F, R158L, Y163C, V173L, V173M, Y205C, Y220C, G245C, R249M, V272M, R273L, and E286K. In some embodiments, the TP53 mutation includes one or more of the mutations above.

In some embodiments, a CREBBP mutation includes, but is not limited to, R1446C, R1446H, S1680del, I1084Sfs*15, P1948L, I1084Nfs*3, ?R386*, S893L, R1341*, P1423Lfs*36, P1488L, Y1503H, R1664C, A1824T, R1173*, R1360*, Y1450C, H2228D, S71L, P928=, D1435N, W1502C, Y1503D, R483*, R601Q, S945L, R1103*, R1288W, R1392*, C1408Y, D1435G, R1446L, H1485Y, Q1491K, Q96*, L361M, L524Wfs*6, Q540*, Q1073*, A1100V, R1169C, C1237Y, R1347W, G1411E, W1472C, I1483F, P1488T, R1498*, Y1503F, Q1856*, R1985C, R2104C, S2328L, V2349=, S2377L, and any combination thereof.

In some embodiments, a KMT2C mutation includes, but is not limited to, D348N, P350=, R380L, C391*, P309S, C988F, Y987H, S990G, K2797Rfs*26, V346=, R894Q, R284Q, S806=, R1690=, P986=, A1685S, G315S, Q755*, R909K, T316S, S772L, G838S, L291F, P335=, C988F, Q2680=, E765G, K339N, Y816*, R526P, N729D, G845E, I817Nfs*11, G892R, C1103*, S3660L, F4496Lfs*21, G315C, R886C, D348N, S793=, V919L, R2481S, R2884*, R4549C, M305Dfs*28, T316S, P377=, I455M, T820I, S965=, S730Y, P860S, Q873Hfs*40, R904*, R2610Q, R4478*, and any combination thereof.

In some embodiments, a KMT2D mutation includes, but is not limited to, L1419P, E640D, E541D, E455D, T2131P, K1420R. P2354Lfs*30, G2493=, Q3612=, 1942=, T1195Hfs*17, P4170=, P1194H, G1235Vfs*95, P4563=, P647Hfs*283, L449_P457del, P3557=, Q3603=, R1702*, P648Tfs*2, R5501*, R4198*, R4484*, R83Q, R1903*, R2685*, R4282*, L5326=, R5432W, R2734*, Q2800*, R2830*, Q3745dup, S4010P, R4904*, G5182Afs*61, R5214H, R1615*, Q2380*, R2687*, R2771*, V3089Wfs*30, Q3799Gfs*212, R4536*, R5030C, R5048C, R5432Q, A221Lfs*40, A476T, A2119Lfs*25, P2557L, R2801*, Q3913*, R4420W, G4641=, R5097*, and any combination thereof.

In some embodiments, a ARID1A mutation includes, but is not limited to, For example, subject has a mutation of ARID1A selected from the group consisting of a C884* (*: nonsense mutation), E966K, Q1411*, F1720fs (fs: frameshift), G1847fs, C1874fs, D1957E, Q1430, R1721fs, G1255E, G284fs, R1722*, M274fs, G1847fs, P559fs, R1276*, Q2176fs, H203fs, A591fs, Q1322*, S2264*, Q586*, Q548fs, and N756fs.

In some embodiments, a RB1 mutation includes, but is not limited to, R320X, R467X, R579X, R455X, R358X, R251X, R787X, R552X, R255X, R556X, Y790X, Q575X, E323X, R661W, R579*, R455*, R556*, R787*, R661W, R445*, R467*, Q217*, Q471*, W195*, Q395*, I680T, E137*, R255*, Q344*, Q62*, E440K, A488V, P777Lfs*33, E322K, R656W, G617Rfs*36, C221*, E440*, Q93*, Q504*, E125*, S834*, E323*, Q685*, S829*, W516*, G435*, Q257*, E79*, S567L, V654M, V654Sfs*14, G100Efs*11, K715*, and any combination thereof.

In some embodiments, an ATM mutation is a mutation in the ATM gene sequence including, but is not limited to, 10744A>G; 10744A>G; 11482G>A; IVS3-558A>T; 146C>G; 381delA; IVS8-3delGT; 1028delAAAA; 1120C>T; 1930ins16; IVS16+2T>C; 2572T>C; IVS21+1G>A; 3085delA; 3381delTGAC; 3602delTT; 4052delT; 4396C>T; 5188C>T; 5290delC; 5546delT; 5791G>CCT; 6047A>G; IVS44-1G>T; 6672delGC/6677delTACG; 6736dell 1/6749del7; 7159insAGCC; 7671delGTTT; 7705del14; 7865C>T; 7979delTGT; 8177C>T; 8545C>T; 8565T>A; IVS64+1G>T; and 9010del28.

In some embodiments of the present invention, a SETD2 mutation is an alteration in the gene sequence encoding the SETD2 protein, when the transcription initiation codon position of the mRNA sequence of NCBI accession number NM_014159 is set to 1. In some embodiments, the 7558th G (guanine) is substituted with T (thymine), the 4774th C (cytosine) is substituted by T, the 1210th A (adenine) is substituted by T, the 4883th T is substituted by G, the 5290th C is replaced by T, the 7072th C is replaced by T, the 4144th G is substituted by T, the 1297 C is replaced by T, the 755th T is replaced by G, the 7261 T is substituted by G, 6700 is replaced by T, the 2536th C is substituted by T, the 7438th C is replaced by T substitution, or there is an insertion of A at position 3866, insertion of T at position 6712, insertion of T at position 7572, deletion of the 913th A, deletion of the 5619th C, deletion of bases 4603-4604, deletion of the 1st base, deletion of the 1936th C, deletion of the 3094-3118 base, insertion of A in the 5289th position, and deletion of the 6323-6333 base.

In some embodiments, a FLT3 mutation includes, but is not limited to, (Q569_E648)ins, D835X, (Q569_E648)delins, (D835_I836), D835Y, D835V, D835Y, D835H, T227M, I836del, N676K, D835E, Y597_E598insDYVDFREY, D835E, D835del, F594_D600dup, A680V, D839G, D96=, D835H, V491L, D835E, Q989*, D835V, L561=, I836del, P986Afs*27, D7G, D324N, S451F, D835N, L576P, Y597_E598insDVDFREY, V491L, N841T, D324N, Y572C, R595_L601dup, K663R, N676K, F691L, D835A, I836H, N841K, S993L, L832F, I836M, A66V, and any combination thereof.

In some embodiments, a PTPN11 mutation includes, but is not limited to, c E76K, A72V, A72T, D61Y, D61V, G60V, E69K, E76G, G507V, S506L, G507A, T73I, E76A, E76Q, S506P, D61N, F71L, E76V, F71L, A72D, V432M, T472M, P495L, N58Y, F285S, S506A, S189A, A465T, R502W, G507R, T511K, D61H, D61G, G507E, G60R, G60A, Q514L, E139D, Y197*, N308D, Q514H, Q514H, N58S, E123D, L206=, A465G, P495S, G507R, and any combination thereof.

In some embodiments, a FGFR1 mutation includes, but is not limited to, N577K, K687E, N577K, D166del, T371M, R476W, T350=, E498K, N577D, D683G, R87C, A154D, N303=, A374V, D550=, S633=, V695L, G728=, R765W, P803S, W19C, P56=, R113C, V149I, S158L, D166dupR220C, N224Kfs*8, D249N, R281W, R281Q, A299S, S424L, S461F, S467F, R506Q, and any combination thereof.

In some embodiments, a EP300 mutation includes, but is not limited to, D1399N, Y1414C, M1470Cfs*26, Y1111*, H2324Pfs*55, R1627W, N2209Q2213delinsK, Q2268del, L415P, M1470Nfs*3, E1514K, C1201Y, P1452L, S952*, C1164Y, D1399Y, S507G, Q824*, D1507N, H2324Tfs*29, P925T, P1440L, W1466C, P1502L, A1629V, R1645*, N1700Tfs*9, P1869L, Q65*, A171V, R202*, R580Q, A627V, Q1082*, N1236Kfs*2, N1286S, R1312*, R1356*, C1385F, H1451L, R1462*, Y1467N, Y1467H, R1478H, R1627Q, R86*, R370H, R397*, R754C, P842S, I997V, E1014*, and any combination thereof.

In some embodiments, a MYC mutation includes, but is limited to, E61T, E681, R74Q, R75N, W135E, W136E, V394D, L420P, W96E, V325D, L351P, a MYC protein with 41 amino acid deleted at the N-terminus (dN2MYC), N26S, S161L, P74L, V7M, F153S, E54D, P246, L164V, P74S, A59V, T73I, P72T, T73A, H374R, P17S, T73N, S264N, P72S, Q52del, S21T, P74A, S107N, P75S, S77P, P261S, P74Q, S190R, A59T, F153C, P75H, T73I, S77F, N11S, S21N, P78L, P72L, N9K, S190N, S267F, T73P, P78S, G105D, S187C, L71M, Q10H, L191x, Q50x, L191F, R25K, F130L, Y27S, D195N, D2G, V20A, V6G, V20I, D2H, P75A, G152D, P74T, C40Y, E8K, Q48x, and any combination thereof.

In some embodiments, a EZH2 mutation is associated with altered histone methylation patterns. In some embodiments, the EZH2 mutation leads to the conversion of amino acid Y641 (equivalent to Y646, catalytic domain), to either F, N, H, S or C resulting in hypertrimethylation of H3K27 and drives lymphomagenesis. In some embodiments, the EZH2 mutation includes EZH2 SET-domain mutations, overexpression of EZH2, overexpression of other PRC2 subunits, loss of function mutations of histone acetyl transferases (HATs), and loss of function of MLL2. Cells that are heterozygous for EZH2 Y646 mutations result in hypertrimethylation of H3K27 relative to cells that are homozygous wild-type (WT) for the EZH2 protein, or to cells that are homozygous for the Y646 mutation.

In some embodiments, a EZH2 mutation includes, but is not limited to, Y646F, Y646N, D185H, Y646F, Y646S, Y646H, R690H, Y646X, E745K, Y646C, V626M, V679M, R690H, R684H, A682G, E249K, G159R, R288Q, N322S, A692V, R690C. D730* (insertion frameshift), S695L, R684C, M667T, R288*, S644*, D192N, K550T, Q653E, D664G, R347Q, Y646C, G660R, R213C, A255T, S538L, N693K, 155M, R561H, A692V, K515R, Y733*, R63*, Q570*, Q328*, R25Q, T467P A656V, T573I, C571Y, E725K, R16W, P577L, F145S, V680M, G686D, G135R, K634E, S652F, R298C, G648E, R566H, L149R, R502Q, Y731D, R313W, N675K, S652C, T374Hfs*3, N152Ifs*15, E401Kfs*22, K406Mfs*17, E246*, S624C, I146T, V626M, L674S, H694R, A581S, and any combination thereof.

In some embodiments, a JAK2 mutation is a mutation in the JAK2 gene includes, but is not limited to, T1923C mutation in combination with a G1920T mutation, a G1920T/C1922T mutation, or a G1920A mutation. In some embodiments, the JAK2 mutation is a mutant JAK2 protein comprising one or more substitutions include, but are not limited to, V617F, V617I, R683G, N542_E543del, E543_D544del, R683S, R683X, F537_K539delinsL (deletion in frame), K539L, N1108S, R1113H, R1063H, R487C, T540Mfs*3 (deletion-frameshift), R867Q, K539L, G571S, R1113C, R938Q, R228Q, L830*, E1080*, K539L, C618R, R564Q, D1036H, L1088S, H538Nfs*4, D873N, V392M, I682F, L393V, M535I, C618R, T875N, L611V, D319N, L611S, G921S, H538Y, S1035L, and any combination thereof.

In some embodiments, a FBXW7 mutation is a point mutation selected from the group consisting of W244* (*: stop codon), R222*, R278*, E192A, S282*, E113D, R465H/C, 726+1 G>A splice, R505C, R479Q, R465C, R367*, R499Vfs*25 (fs*: frameshift), R658*, D600Y, D520N, D520Y, and any combination thereof. In further embodiments, the FBXW7 mutation is double- or triple-mutation includes, but is not limited to, R479Q and S582L, R465H and S582L, D520N, D520Y and R14Q, and R367* and S582L.

In some embodiments, a CCND3 mutation includes, but is not limited to, S259A, R271Pfs*53 (insertion-caused frameshift), E51*, Q260*, P199S, T283A, T283P, V287D, D286_T288del, R271Gfs*33, Q276*, R241Q, D238G, R33P, I290K, I290T, I290R, P267fs, P284S, P284L, P100S, E253D, S262I, R14W, R114L, D238N, A266E, R167W, and any combination thereof.

In some embodiments, a GNA11 mutation includes, but is not limited to, Q209L, R183C, T257=, R183C, G208Afs*16, Q209H, R183C, Q209P, Q209R, Q209H, ?T96=, R210W, R256Q, T334=, G48D, S53G, Q209P, R213Q, and any combination thereof. In some embodiments, the GNA11 mutation has two mutations in exon 4, e.g., a mutation in V182 and a mutation in T175, or one or more mutations in exon 5.

2. Combinations with PD-1 and PD-L1 Inhibitors

In some embodiments, the TIL therapy provided to patients with cancer may include treatment with therapeutic populations of TILs alone or may include a combination treatment including TILs and one or more PD-1 and/or PD-L1 inhibitors.

Programmed death 1 (PD-1) is a 288-amino acid transmembrane immunocheckpoint receptor protein expressed by T cells, B cells, natural killer (NK) T cells, activated monocytes, and dendritic cells. PD-1, which is also known as CD279, belongs to the CD28 family, and in humans is encoded by the Pdcd1 gene on chromosome 2. PD-1 consists of one immunoglobulin (Ig) superfamily domain, a transmembrane region, and an intracellular domain containing an immunoreceptor tyrosine-based inhibitory motif (ITM) and an immunoreceptor tyrosine-based switch motif (ITSM). PD-1 and its ligands (PD-L1 and PD-L2) are known to play a key role in immune tolerance, as described in Keir, et al., Annu. Rev. Immunol. 2008, 26, 677-704. PD-1 provides inhibitory signals that negatively regulate T cell immune responses. PD-L1 (also known as B7-H1 or CD274) and PD-L2 (also known as B7-DC or CD273) are expressed on tumor cells and stromal cells, which may be encountered by activated T cells expressing PD-1, leading to immunosuppression of the T cells. PD-L1 is a 290 amino acid transmembrane protein encoded by the Cd274 gene on human chromosome 9. Blocking the interaction between PD-1 and its ligands PD-L1 and PD-L2 by use of a PD-1 inhibitor, a PD-L1 inhibitor, and/or a PD-L2 inhibitor can overcome immune resistance, as demonstrated in recent clinical studies, such as that described in Topalian, et al., N. Eng. J. Med. 2012, 366, 2443-54. PD-L1 is expressed on many tumor cell lines, while PD-L2 is expressed is expressed mostly on dendritic cells and a few tumor lines. In addition to T cells (which inducibly express PD-1 after activation), PD-1 is also expressed on B cells, natural killer cells, macrophages, activated monocytes, and dendritic cells.

In an embodiment, the PD-1 inhibitor may be any PD-1 inhibitor or PD-1 blocker known in the art. In particular, it is one of the PD-1 inhibitors or blockers described in more detail in the following paragraphs. The terms “inhibitor,” “antagonist,” and “blocker” are used interchangeably herein in reference to PD-1 inhibitors. For avoidance of doubt, references herein to a PD-1 inhibitor that is an antibody may refer to a compound or antigen-binding fragments, variants, conjugates, or biosimilars thereof. For avoidance of doubt, references herein to a PD-1 inhibitor may also refer to a small molecule compound or a pharmaceutically acceptable salt, ester, solvate, hydrate, cocrystal, or prodrug thereof.

In a preferred embodiment, the PD-1 inhibitor is an antibody (i.e., an anti-PD-1 antibody), a fragment thereof, including Fab fragments, or a single-chain variable fragment (scFv) thereof. In some embodiments the PD-1 inhibitor is a polyclonal antibody. In a preferred embodiment, the PD-1 inhibitor is a monoclonal antibody. In some embodiments, the PD-1 inhibitor competes for binding with PD-1, and/or binds to an epitope on PD-1. In an embodiment, the antibody competes for binding with PD-1, and/or binds to an epitope on PD-1.

In some embodiments, the PD-1 inhibitor is one that binds human PD-1 with a KD of about 100 pM or lower, binds human PD-1 with a KD of about 90 pM or lower, binds human PD-1 with a KD of about 80 pM or lower, binds human PD-1 with a KD of about 70 pM or lower, binds human PD-1 with a KD of about 60 pM or lower, binds human PD-1 with a KD of about 50 pM or lower, binds human PD-1 with a KD of about 40 pM or lower, binds human PD-1 with a KD of about 30 pM or lower, binds human PD-1 with a KD of about 20 pM or lower, binds human PD-1 with a KD of about 10 pM or lower, or binds human PD-1 with a KD of about 1 pM or lower.

In some embodiments, the PD-1 inhibitor is one that binds to human PD-1 with a k_{assoc} of about 7.5×10⁵ l/M·s or faster, binds to human PD-1 with a k_{assoc} of about 7.5×10⁵ l/M·s or faster, binds to human PD-1 with a k_{assoc} of about 8×10⁵ l/M·s or faster, binds to human PD-1 with a k_{assoc} of about 8.5×10⁵ l/M·s or faster, binds to human PD-1 with a k_{assoc} of about 9×10⁵ l/M·s or faster, binds to human PD-1 with a k_{assoc} of about 9.5×10⁵ l/M·s or faster, or binds to human PD-1 with a k_{assoc} of about 1×10⁶ l/M·s or faster.

In some embodiments, the PD-1 inhibitor is one that binds to human PD-1 with a k_{dissoc} of about 2×10-5 l/s or slower, binds to human PD-1 with a k_{dissoc} of about 2.1×10-5 l/s or slower, binds to human PD-1 with a k_{dissoc} of about 2.2×10-5 l/s or slower, binds to human PD-1 with a k_{dissoc} of about 2.3×10-5 l/s or slower, binds to human PD-1 with a k_{dissoc} of about 2.4×10-5 l/s or slower, binds to human PD-1 with a k_{dissoc} of about 2.5×10-5 l/s or slower, binds to human PD-1 with a k_{dissoc} of about 2.6×10-5 l/s or slower or binds to human PD-1 with a k_{dissoc} of about 2.7×10-5 l/s or slower, binds to human PD-1 with a k_{dissoc} of about 2.8×10-5 l/s or slower, binds to human PD-1 with a k_{dissoc} of about 2.9×10-5 l/s or slower, or binds to human PD-1 with a k_{dissoc} of about 3×10-5 l/s or slower.

In some embodiments, the PD-1 inhibitor is one that blocks or inhibits binding of human PD-L1 or human PD-L2 to human PD-1 with an IC50 of about 10 nM or lower, blocks or inhibits binding of human PD-L1 or human PD-L2 to human PD-1 with an IC50 of about 9 nM or lower, blocks or inhibits binding of human PD-L1 or human PD-L2 to human PD-1 with an IC50 of about 8 nM or lower, blocks or inhibits binding of human PD-L1 or human PD-L2 to human PD-1 with an IC50 of about 7 nM or lower, blocks or inhibits binding of human PD-L1 or human PD-L2 to human PD-1 with an IC50 of about 6 nM or lower, blocks or inhibits binding of human PD-L1 or human PD-L2 to human PD-1 with an IC50 of about 5 nM or lower, blocks or inhibits binding of human PD-L1 or human PD-L2 to human PD-1 with an IC50 of about 4 nM or lower, blocks or inhibits binding of human PD-L1 or human PD-L2 to human PD-1 with an IC50 of about 3 nM or lower, blocks or inhibits binding of human PD-L1 or human PD-L2 to human PD-1 with an IC50 of about 2 nM or lower, or blocks or inhibits binding of human PD-L1 or human PD-L2 to human PD-1 with an IC50 of about 1 nM or lower.

In an embodiment, the PD-1 inhibitor is nivolumab (commercially available as OPDIVO from Bristol-Myers Squibb Co.), or biosimilars, antigen-binding fragments, conjugates, or variants thereof. Nivolumab is a fully human IgG4 antibody blocking the PD-1 receptor. In an embodiment, the anti-PD-1 antibody is an immunoglobulin G4 kappa, anti-(human CD274) antibody. Nivolumab is assigned Chemical Abstracts Service (CAS) registry number 946414-94-4 and is also known as 5C4, BMS-936558, MDX-1106, and ONO-4538. The preparation and properties of nivolumab are described in U.S. Pat. No. 8,008,449 and International Patent Publication No. WO 2006/121168, the disclosures of which are incorporated by reference herein. The clinical safety and efficacy of nivolumab in various forms of cancer has been described in Wang, et al., Cancer Immunol. Res. 2014, 2, 846-56; Page, et al., Ann. Rev. Med., 2014, 65, 185-202; and Weber, et al., J Clin. Oncology, 2013, 31, 4311-4318, the disclosures of which are incorporated by reference herein. The amino acid sequences of nivolumab are set forth in Table 18. Nivolumab has intra-heavy chain disulfide linkages at 22-96, 140-196, 254-314, 360-418, 22″-96″, 140″-196″, 254″-314″, and 360″-418″; intra-light chain disulfide linkages at 23′-88′, 134′-194′, 23′″-88′″, and 134′″-194′″; inter-heavy-light chain disulfide linkages at 127-214′, 127″-214′″, inter-heavy-heavy chain disulfide linkages at 219-219″ and 222-222″; and N-glycosylation sites (H CH2 84.4) at 290, 290″.

In an embodiment, a PD-1 inhibitor comprises a heavy chain given by SEQ ID NO:158 and a light chain given by SEQ ID NO:159. In an embodiment, a PD-1 inhibitor comprises heavy and light chains having the sequences shown in SEQ ID NO:158 and SEQ ID NO:159, respectively, or antigen binding fragments, Fab fragments, single-chain variable fragments (scFv), variants, or conjugates thereof. In an embodiment, a PD-1 inhibitor comprises heavy and light chains that are each at least 99% identical to the sequences shown in SEQ ID NO:158 and SEQ ID NO:159, respectively. In an embodiment, a PD-1 inhibitor comprises heavy and light chains that are each at least 98% identical to the sequences shown in SEQ ID NO:158 and SEQ ID NO:159, respectively. In an embodiment, a PD-1 inhibitor comprises heavy and light chains that are each at least 97% identical to the sequences shown in SEQ ID NO:158 and SEQ ID NO:159, respectively. In an embodiment, a PD-1 inhibitor comprises heavy and light chains that are each at least 96% identical to the sequences shown in SEQ ID NO:158 and SEQ ID NO:159, respectively. In an embodiment, a PD-1 inhibitor comprises heavy and light chains that are each at least 95% identical to the sequences shown in SEQ ID NO:463 and SEQ ID NO:159, respectively.

In an embodiment, the PD-1 inhibitor comprises the heavy and light chain CDRs or variable regions (VRs) of nivolumab. In an embodiment, the PD-1 inhibitor heavy chain variable region (V_{H}) comprises the sequence shown in SEQ ID NO:160, and the PD-1 inhibitor light chain variable region (V_{L}) comprises the sequence shown in SEQ ID NO:161, or conservative amino acid substitutions thereof. In an embodiment, a PD-1 inhibitor comprises V_{H} and V_{L} regions that are each at least 99% identical to the sequences shown in SEQ ID NO:160 and SEQ ID NO:161, respectively. In an embodiment, a PD-1 inhibitor comprises V_{H} and V_{L} regions that are each at least 98% identical to the sequences shown in SEQ ID NO:160 and SEQ ID NO:161, respectively. In an embodiment, a PD-1 inhibitor comprises V_{H} and V_{L} regions that are each at least 97% identical to the sequences shown in SEQ ID NO:160 and SEQ ID NO:161, respectively. In an embodiment, a PD-1 inhibitor comprises V_{H} and V_{L} regions that are each at least 96% identical to the sequences shown in SEQ ID NO:160 and SEQ ID NO:161, respectively. In an embodiment, a PD-1 inhibitor comprises V_{H} and V_{L} regions that are each at least 95% identical to the sequences shown in SEQ ID NO:160 and SEQ ID NO:161, respectively.

In an embodiment, a PD-1 inhibitor comprises heavy chain CDR1, CDR2 and CDR3 domains having the sequences set forth in SEQ ID NO:162, SEQ ID NO:163, and SEQ ID NO:164, respectively, or conservative amino acid substitutions thereof, and light chain CDR1, CDR2 and CDR3 domains having the sequences set forth in SEQ ID NO:165, SEQ ID NO:166, and SEQ ID NO:167, respectively, or conservative amino acid substitutions thereof. In an embodiment, the antibody competes for binding with, and/or binds to the same epitope on PD-1 as any of the aforementioned antibodies.

In an embodiment, the PD-1 inhibitor is an anti-PD-1 biosimilar monoclonal antibody approved by drug regulatory authorities with reference to nivolumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is nivolumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is nivolumab. The ant-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab.

TABLE 18
Amino acid sequences for PD-1 inhibitors related to nivolumab.

| Identifier | Sequence (One-Letter Amino Acid Symbols) | |
|---|---|---|
| SEQ ID NO: 463 | QVOLVESGGG VVQPGRSLRL DCKASGITFS NSGMHWVRQA PGKGLEWVAV IWYDGSKRYY | 60 |
| nivolumab heavy | ADSVKGRFTI SRDNSKNTLF LOMNSLRAED TAVYYCATND DYWGQGTLVT VSSASTKGPS | 120 |
| chain | VFPLAPCSRS TSESTAALGC LVKDYFPEPV TVSWNSGALT SGVHTFPAVL QSSGLYSLSS | 180 |
| | VVTVPSSSLG TKTYTCNVDH KPSNTKVDKR VESKYGPPCP PCPAPEFLGG PSVFLFPPKP | 240 |
| | KDTIMISRTP EVTCVVVDVS QEDPEVQFNW YVDGVEVHNA KTKPREEQFN STYRVVSVLT | 300 |
| | VLHQDWLNGK EYKCKVSNKG LPSSIEKTIS KAKGOPREPQ VYTLPPSQEE MTKNQVSLTC | 360 |
| | LVKGFYPSDI AVEWESNGOP ENNYKTTPPV LDSDGSFFLY SRLTVDKSRW QEGNVFSCSV | 420 |
| | MHEALHNHYT QKSLSLSLGK | 440 |
| SEQ ID NO: 159 | EIVITQSPAT LSLSPGERAT LSCRASQSVS SYLAWYQQKP GOAPRLLIYD ASNRATGIPA | 60 |
| nivolumab light | RFSGSGSGTD FTLT_SSLEP EDFAVYYCQQ SSNWPRTFGQ GTKVEIKRTV AAPSVFIFPP | 120 |
| chain | SDEQLKSGTA SVVCLLNNFY PREAKVOWKV DNALQSGNSQ ESVTEQDSKD STYSLSSTLT | 180 |
| | LSKADYEKHK VYACEVTHQG LSSPVTKSEN RGEC | 214 |
| SEQ ID NO: 160 | QVOLVESGGG VVQPGRSLRL DCKASGITFS NSGMHWVRQA PGKGLEWVAV IWYDGSKRYY | 60 |
| nivolumab | | |
| variable heavy | ADSVKGRFTI SRDNSKNTLF LOMNSLRAED TAVYYCATND DYWGQGTLVT VSS | 113 |
| chain | | |
| SEQ ID NO: 161 | EIVITQSPAT LSLSPGERAT LSCRASQSVS SYLAWYQQKP GQAPRLLIYD ASNRATGIPA | 60 |
| nivolumab | | |
| variable light | RFSGSGSGTD FTLT_SSLEP EDFAVYYCQQ SSNWPRTFGQ GTKVEIK | 107 |
| chain | | |
| SEQ ID NO: 162 | NSGMH | 5 |
| nivolumab heavy | | |
| chain CDR1 | | |
| SEQ ID NO: 163 | VIWYDGSKRY YADSVKG | 17 |
| nivolumab heavy | | |
| chain CDR2 | | |
| SEQ ID NO: 164 | NDDY | 4 |
| nivolumab heavy | | |
| chain CDR3 | | |
| SEQ ID NO: 165 | RASQSVSSYL A | 11 |
| nivolumab light | | |
| chain CDR1 | | |
| SEQ ID NO: 166 | DASNRAT | 7 |
| nivolumab light | | |
| chain CDR2 | | |
| SEQ ID NO: 167 | QQSSNWPRT | 9 |
| nivolumab light | | |
| chain CDR3 | | |

In some embodiments, the PD-1 inhibitor is nivolumab or a biosimilar thereof, and the nivolumab is administered at a dose of about 0.5 mg/kg to about 10 mg/kg. In some embodiments, the PD-1 inhibitor is nivolumab or a biosimilar thereof, and the nivolumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the PD-1 inhibitor is nivolumab or a biosimilar thereof, and the nivolumab is administered at a dose of about 200 mg to about 500 mg. In some embodiments, the PD-1 inhibitor is nivolumab or a biosimilar thereof, and the nivolumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the PD-1 inhibitor is nivolumab or a biosimilar thereof, and the nivolumab is administered every 2 weeks, every 3 weeks, every 4 weeks, every 5 weeks, or every 6 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat unresectable or metastatic melanoma. In some embodiments, the nivolumab is administered to treat unresectable or metastatic melanoma and is administered at about 240 mg every 2 weeks. In some embodiments, the nivolumab is administered to treat unresectable or metastatic melanoma and is administered at about 480 mg every 4 weeks. In some embodiments, the nivolumab is administered to treat unresectable or metastatic melanoma and is administered at about 1 mg/kg followed by ipilimumab 3 mg/kg on the same day every 3 weeks for 4 doses, then 240 mg every 2 weeks or 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered for the adjuvant treatment of melanoma. In some embodiments, the nivolumab is administered for the adjuvant treatment of melanoma at about 240 mg every 2 weeks. In some embodiments, the nivolumab is administered for the adjuvant treatment of melanoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat metastatic non-small cell lung cancer. In some embodiments, the nivolumab is administered to treat metastatic non-small cell lung cancer at about 3 mg/kg every 2 weeks along with ipilimumab at about 1 mg/kg every 6 weeks. In some embodiments, the nivolumab is administered to treat metastatic non-small cell lung cancer at about 360 mg every 3 weeks with ipilimumab 1 mg/kg every 6 weeks and 2 cycles of platinum-doublet chemotherapy. In some embodiments, the nivolumab is administered to treat metastatic non-small cell lung cancer at about 240 mg every 2 weeks or 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat small cell lung cancer. In some embodiments, the nivolumab is administered to treat small cell lung cancer at about 240 mg every 2 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat malignant pleural mesothelioma at about 360 mg every 3 weeks with ipilimumab 1 mg/kg every 6 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat advanced renal cell carcinoma. In some embodiments, the nivolumab is administered to treat advanced renal cell carcinoma at about 240 mg every 2 weeks. In some embodiments, the nivolumab is administered to treat advanced renal cell carcinoma at about 480 mg every 4 weeks. In some embodiments, the nivolumab is administered to treat advanced renal cell carcinoma at about 3 mg/kg followed by ipilimumab at about 1 mg/kg on the same day every 3 weeks for 4 doses, then 240 mg every 2 weeks. In some embodiments, the nivolumab is administered to treat advanced renal cell carcinoma at about 3 mg/kg followed by ipilimumab at about 1 mg/kg on the same day every 3 weeks for 4 doses, then 240 mg every 2 weeks 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat classical Hodgkin lymphoma. In some embodiments, the nivolumab is administered to treat classical Hodgkin lymphoma at about 240 mg every 2 weeks. In some embodiments, the nivolumab is administered to treat classical Hodgkin lymphoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat Recurrent or metastatic squamous cell carcinoma of the head and neck. In some embodiments, the nivolumab is administered to treat recurrent or metastatic squamous cell carcinoma of the head and neck at about 240 mg every 2 weeks. In some embodiments, the nivolumab is administered to treat recurrent or metastatic squamous cell carcinoma of the head and neck at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat locally advanced or metastatic urothelial carcinoma at about 240 mg every 2 weeks. In some embodiments, the nivolumab is administered to treat locally advanced or metastatic urothelial carcinoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat microsatellite instability-high (MSI-H) or mismatch repair deficient (dMMR) metastatic colorectal cancer. In some embodiments, the nivolumab is administered to treat microsatellite instability-high (MSI-H) or mismatch repair deficient (dMMR) metastatic colorectal cancer in adult and pediatric patients. In some embodiments, the nivolumab is administered to treat microsatellite instability-high (MSI-H) or mismatch repair deficient (dMMR) metastatic colorectal cancer in adult and pediatric patients 40 kg at about 240 mg every 2 weeks. In some embodiments, the nivolumab is administered to treat microsatellite instability-high (MSI-H) or mismatch repair deficient (dMMR) metastatic colorectal cancer in adult and pediatric patients 240 kg at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat microsatellite instability-high (MSI-H) or mismatch repair deficient (dMMR) metastatic colorectal cancer in pediatric patients <40 kg at about 3 mg/kg every 2 weeks. In some embodiments, the nivolumab is administered to treat microsatellite instability-high (MSI-H) or mismatch repair deficient (dMMR) metastatic colorectal cancer in adult and pediatric patients 40 kg at about 3 mg/kg followed by ipilimumab 1 mg/kg on the same day every 3 weeks for 4 doses, then 240 mg every 2 weeks. In some embodiments, the nivolumab is administered to treat microsatellite instability-high (MSI-H) or mismatch repair deficient (dMMR) metastatic colorectal cancer in adult and pediatric patients ≥40 kg at about 3 mg/kg followed by ipilimumab 1 mg/kg on the same day every 3 weeks for 4 doses, then 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat hepatocellular carcinoma. In some embodiments, the nivolumab is administered to treat hepatocellular carcinoma at about 240 mg every 2 weeks. In some embodiments, the nivolumab is administered to treat hepatocellular carcinoma at about 480 mg every 4 weeks. In some embodiments, the nivolumab is administered to treat hepatocellular carcinoma at about 1 mg/kg followed by ipilimumab 3 mg/kg on the same day every 3 weeks for 4 doses, then 240 mg every 2 weeks. In some embodiments, the nivolumab is administered to treat hepatocellular carcinoma at about 1 mg/kg followed by ipilimumab 3 mg/kg on the same day every 3 weeks for 4 doses, then 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat esophageal squamous cell carcinoma. In some embodiments, the nivolumab is administered to treat esophageal squamous cell carcinoma at about 240 mg every 2 weeks. In some embodiments, the nivolumab is administered to treat esophageal squamous cell carcinoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In another embodiment, the PD-1 inhibitor comprises pembrolizumab (commercially available as KEYTRUDA from Merck & Co., Inc., Kenilworth, NJ, USA), or antigen-binding fragments, conjugates, or variants thereof. Pembrolizumab is assigned CAS registry number 1374853-91-4 and is also known as lambrolizumab, MK-3475, and SCH-900475. Pembrolizumab has an immunoglobulin G4, anti-(human protein PDCD1 (programmed cell death 1)) (human-Mus musculus monoclonal heavy chain), disulfide with human-Mus musculus monoclonal light chain, dimer structure. The structure of pembrolizumab may also be described as immunoglobulin G4, anti-(human programmed cell death 1); humanized mouse monoclonal [228-L-proline(H10-S>P)]γ4 heavy chain (134-218′)-disulfide with humanized mouse monoclonal κ light chain dimer (226-226″:229-229″)-bisdisulfide. The properties, uses, and preparation of pembrolizumab are described in International Patent Publication No. WO 2008/156712 A1, U.S. Pat. No. 8,354,509 and U.S. Patent Application Publication Nos. US 2010/0266617 A1, US 2013/0108651 A1, and US 2013/0109843 A2, the disclosures of which are incorporated herein by reference. The clinical safety and efficacy of pembrolizumab in various forms of cancer is described in Fuerst, Oncology Times, 2014, 36, 35-36; Robert, et al., Lancet, 2014, 384, 1109-17; and Thomas, et al., Exp. Opin. Biol. Ther., 2014, 14, 1061-1064. The amino acid sequences of pembrolizumab are set forth in Table 19. Pembrolizumab includes the following disulfide bridges: 22-96, 22″-96″, 23′-92′, 23′″-92′″, 134-218′, 134″-218′″, 138′-198′, 138′″-198′″, 147-203, 147″-203″, 226-226″, 229-229″, 261-321, 261″-321″, 367-425, and 367″-425″, and the following glycosylation sites (N): Asn-297 and Asn-297″. Pembrolizumab is an IgG4/kappa isotype with a stabilizing S228P mutation in the Fc region; insertion of this mutation in the IgG4 hinge region prevents the formation of half molecules typically observed for IgG4 antibodies. Pembrolizumab is heterogeneously glycosylated at Asn297 within the Fc domain of each heavy chain, yielding a molecular weight of approximately 149 kDa for the intact antibody. The dominant glycoform of pembrolizumab is the fucosylated agalacto diantennary glycan form (GOF).

In an embodiment, a PD-1 inhibitor comprises a heavy chain given by SEQ ID NO:168 and a light chain given by SEQ ID NO:169. In an embodiment, a PD-1 inhibitor comprises heavy and light chains having the sequences shown in SEQ ID NO:168 and SEQ ID NO:169, respectively, or antigen binding fragments, Fab fragments, single-chain variable fragments (scFv), variants, or conjugates thereof. In an embodiment, a PD-1 inhibitor comprises heavy and light chains that are each at least 99% identical to the sequences shown in SEQ ID NO:168 and SEQ ID NO:169, respectively. In an embodiment, a PD-1 inhibitor comprises heavy and light chains that are each at least 98% identical to the sequences shown in SEQ ID NO:168 and SEQ ID NO:169, respectively. In an embodiment, a PD-1 inhibitor comprises heavy and light chains that are each at least 97% identical to the sequences shown in SEQ ID NO:168 and SEQ ID NO:169, respectively. In an embodiment, a PD-1 inhibitor comprises heavy and light chains that are each at least 96% identical to the sequences shown in SEQ ID NO:168 and SEQ ID NO:169, respectively. In an embodiment, a PD-1 inhibitor comprises heavy and light chains that are each at least 95% identical to the sequences shown in SEQ ID NO:168 and SEQ ID NO:169, respectively.

In an embodiment, the PD-1 inhibitor comprises the heavy and light chain CDRs or variable regions (VRs) of pembrolizumab. In an embodiment, the PD-1 inhibitor heavy chain variable region (V_{H}) comprises the sequence shown in SEQ ID NO:170, and the PD-1 inhibitor light chain variable region (V_{L}) comprises the sequence shown in SEQ ID NO:171, or conservative amino acid substitutions thereof. In an embodiment, a PD-1 inhibitor comprises V_{H} and V_{L} regions that are each at least 99% identical to the sequences shown in SEQ ID NO:170 and SEQ ID NO:171, respectively. In an embodiment, a PD-1 inhibitor comprises V_{H} and V_{L} regions that are each at least 98% identical to the sequences shown in SEQ ID NO:170 and SEQ ID NO:171, respectively. In an embodiment, a PD-1 inhibitor comprises V_{H} and V_{L} regions that are each at least 97% identical to the sequences shown in SEQ ID NO:170 and SEQ ID NO:171, respectively. In an embodiment, a PD-1 inhibitor comprises V_{H} and V_{L} regions that are each at least 96% identical to the sequences shown in SEQ ID NO:170 and SEQ ID NO:171, respectively. In an embodiment, a PD-1 inhibitor comprises V_{H} and V_{L} regions that are each at least 95% identical to the sequences shown in SEQ ID NO:170 and SEQ ID NO:171, respectively.

In an embodiment, a PD-1 inhibitor comprises the heavy chain CDR1, CDR2 and CDR3 domains having the sequences set forth in SEQ ID NO:172, SEQ ID NO:173, and SEQ ID NO:174, respectively, or conservative amino acid substitutions thereof, and light chain CDR1, CDR2 and CDR3 domains having the sequences set forth in SEQ ID NO:175, SEQ ID NO:176, and SEQ ID NO:177, respectively, or conservative amino acid substitutions thereof. In an embodiment, the antibody competes for binding with, and/or binds to the same epitope on PD-1 as any of the aforementioned antibodies.

In an embodiment, the PD-1 inhibitor is an anti-PD-1 biosimilar monoclonal antibody approved by drug regulatory authorities with reference to pembrolizumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pembrolizumab. In some embodiments, the one or more post-translational modifications are selected from one or more of glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pembrolizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab.

TABLE 19
Amino acid sequences for PD-1 inhibitors related to pembrolizumab.

| Identifier | Sequence (One-Letter Amino Acid Symbols) | |
|---|---|---|
| SEQ ID NO: 168 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| heavy chain | ASTKGPSVFP LAPCSRSTSE STAALGCLVK DYFPEPVTVS WNSGALTSGV HTFPAVLQSS | 180 |
| | GLYSLSSVVT VPSSSLGTKT YTCNVDHKPS NTKVDKRVES KYGPPCPPCP APEFLGGPSV | 240 |
| | FLFPPKPKDT LMISRTPEVT CVVVDVSQED PEVQFNWYVD GVEVHNAKTK PREEQFNSTY | 300 |
| | RVVSVLTVLH QDWINGKEYK CKVSNKGLPS SIEKTISKAK GQPREPQVYT LPPSQEEMTK | 360 |
| | NQVSLTCLVK GFYPSDIAVE WESNGQPENN YKTTPPVLDS DGSFFLYSRL TVDKSRWQEG | 420 |
| | NVFSCSVMHE ALHNHYTQKS LSLSLGK | 447 |
| SEQ ID NO: 169 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI KRTVAAPSVE | 120 |
| light chain | IFPPSDEQLK SGTASVVCLL NNFYPREAKV QWKVDNALQS GNSQESVTEQ DSKDSTYSLS | 180 |
| | STITLSKADY EKHKVYACEV THQGLSSPVT KSFNRGEC | 218 |
| SEQ ID NO: 170 | QVQLVQSGVE VKKPGASVKV SCKASGYTFT NYYMYWVRQA PGQGLEWMGG INPSNGGTNE | 60 |
| pembrolizumab | | |
| variable heavy | NEKFKNRVTL TTDSSTTTAY MELKSLQFDD TAVYYCARRD YRFDMGFDYW GQGTTVTVSS | 120 |
| chain | | |
| SEQ ID NO: 171 | EIVLTQSPAT LSLSPGERAT LSCRASKGVS TSGYSYLHWY QQKPGQAPRL LIYLASYLES | 60 |
| pembrolizumab | | |
| variable light | GVPARFSGSG SGTDFTLTIS SLEPEDFAVY YCQHSRDLPL TFGGGTKVEI K | 111 |
| chain | | |
| SEQ ID NO: 172 | NYYMY | 5 |
| pembrolizumab | | |
| heavy chain | | |
| CDR1 | | |
| SEQ ID NO: 173 | GINPSNGGTN FNEKFK | 16 |
| pembrolizumab | | |
| heavy chain | | |
| CDR2 | | |
| SEQ ID NO: 174 | RDYRFDMGED Y | 11 |
| pembrolizumab | | |
| heavy chain | | |
| CDR3 | | |
| SEQ ID NO: 175 | RASKGVSTSG YSYLH | 15 |
| pembrolizumab | | |
| light chain | | |
| CDR1 | | |
| SEQ ID NO: 176 | LASYLES 7 | |
| pembrolizumab | | |
| light chain | | |
| CDR2 | | |
| SEQ ID NO: 177 | QHSRDLPLT | 9 |
| pembrolizumab | | |
| light chain | | |
| CDR3 | | |

In some embodiments, the PD-1 inhibitor is pembrolizumab or a biosimilar thereof, and the pembrolizumab is administered at a dose of about 0.5 mg/kg to about 10 mg/kg. In some embodiments, the PD-1 inhibitor is pembrolizumab or a biosimilar thereof, and the pembrolizumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is pembrolizumab or a biosimilar thereof, wherein the pembrolizumab is administered at a dose of about 200 mg to about 500 mg. In some embodiments, the PD-1 inhibitor is pembrolizumab or a biosimilar thereof, and the nivolumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is pembrolizumab or a biosimilar thereof, wherein the pembrolizumab is administered every 2 weeks, every 3 weeks, every 4 weeks, every 5 weeks, or every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat melanoma. In some embodiments, the pembrolizumab is administered to treat melanoma at about 200 mg every 3 weeks. In some embodiments, the pembrolizumab is administered to treat melanoma at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat NSCLC. In some embodiments, the pembrolizumab is administered to treat NSCLC at about 200 mg every 3 weeks. In some embodiments, the pembrolizumab is administered to treat NSCLC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat small cell lung cancer (SCLC). In some embodiments, the pembrolizumab is administered to treat SCLC at about 200 mg every 3 weeks. In some embodiments, the pembrolizumab is administered to treat SCLC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat head and neck squamous cell cancer (HNSCC). In some embodiments, the pembrolizumab is administered to treat HNSCC at about 200 mg every 3 weeks. In some embodiments, the pembrolizumab is administered to treat HNSCC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat classical Hodgkin lymphoma (cHL) or primary mediastinal large B-cell lymphoma (PMBCL) at about 200 mg every 3 weeks. In some embodiments, the pembrolizumab is administered to treat classical Hodgkin lymphoma (cHL) or primary mediastinal large B-cell lymphoma (PMBCL) at about 400 mg every 6 weeks for adults. In some embodiments, the pembrolizumab is administered to treat classical Hodgkin lymphoma (cHL) or primary mediastinal large B-cell lymphoma (PMBCL) at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat urothelial carcinoma at about 200 mg every 3 weeks. In some embodiments, the pembrolizumab is administered to treat urothelial carcinoma at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat microsatellite instability-high (MSI-H) or mismatch repair deficient (dMMR) cancer at about 200 mg every 3 weeks. In some embodiments, the pembrolizumab is administered to treat MSI-H or dMMR cancer at about 400 mg every 6 weeks for adults. In some embodiments, the pembrolizumab is administered to treat MSI-H or dMMR cancer at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient). In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat microsatellite instability-high (MSI-H) or mismatch repair deficient colorectal cancer (dMMR CRC at about 200 mg every 3 weeks. In some embodiments, the pembrolizumab is administered to treat MSI-H or dMMR CRC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat gastric cancer at about 200 mg every 3 weeks. In some embodiments, the pembrolizumab is administered to treat gastric cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat Esophageal Cancer at about 200 mg every 3 weeks. In some embodiments, the pembrolizumab is administered to treat Esophageal Cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat cervical cancer at about 200 mg every 3 weeks. In some embodiments, the pembrolizumab is administered to treat cervical cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat hepatocellular carcinoma (HCC) at about 200 mg every 3 weeks. In some embodiments, the pembrolizumab is administered to treat HCC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat Merkel cell carcinoma (MCC) at about 200 mg every 3 weeks for adults. In some embodiments, the pembrolizumab is administered to treat MCC at about 400 mg every 6 weeks for adults. In some embodiments, the pembrolizumab is administered to treat MCC at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient). In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat renal cell carcinoma (RCC) at about 200 mg every 3 weeks. In some embodiments, the pembrolizumab is administered to treat RCC at about 400 mg every 6 weeks with axitinib 5 mg orally twice daily. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat endometrial carcinoma at about 200 mg every 3 weeks. In some embodiments, the pembrolizumab is administered to treat endometrial carcinoma at about 400 mg every 6 weeks with lenvatinib 20 mg orally once daily for tumors that are not MSI-H or dMMR. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat tumor mutational burden-high (TMB-H) Cancer at about 200 mg every 3 weeks for adults. In some embodiments, the pembrolizumab is administered to treat TMB-H Cancer at about 400 mg every 6 weeks for adults. In some embodiments, the pembrolizumab is administered to treat TMB-H Cancer at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat cutaneous squamous cell carcinoma (cSCC) at about 200 mg every 3 weeks. In some embodiments, the pembrolizumab is administered to treat cSCC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat triple-negative breast cancer (TNBC) at about 200 mg every 3 weeks. In some embodiments, the pembrolizumab is administered to treat TNBC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In an embodiment, if the patient or subject is an adult, i.e., treatment of adult indications, and additional dosing regimen of 400 mg every 6 weeks can be employed. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In an embodiment, the PD-1 inhibitor or anti-PD-1 antibody is cemiplimab, or a fragment, variant, conjugate, or biosimilar thereof, which is commercially available from Regeneron, Inc. In an embodiment, the PD-1 inhibitor or anti-PD-1 antibody is tislelizumab, or a fragment, variant, conjugate, or biosimilar thereof, which is available from Novartis AG and Beigene Co., Ltd. In an embodiment, the PD-1 inhibitor or anti-PD-1 antibody is sintilimab, or a fragment, variant, conjugate, or biosimilar thereof, which is available from Eli Lilly and Co. In an embodiment, the PD-1 inhibitor or anti-PD-1 antibody is toripalimab, or a fragment, variant, conjugate, or biosimilar thereof, which is available from Junshi Biosciences Co., Ltd. and Coherus BioSciences, Inc. In an embodiment, the PD-1 inhibitor or anti-PD-1 antibody is dostarlimab, or a fragment, variant, conjugate, or biosimilar thereof, which is available from GlaxoSmithKline plc.

In an embodiment, the PD-1 inhibitor is a commercially-available anti-PD-1 monoclonal antibody, such as anti-m-PD-1 clones J43 (Cat #BE0033-2) and RMP1-14 (Cat #BE0146) (Bio X Cell, Inc., West Lebanon, NH, USA). A number of commercially-available anti-PD-1 antibodies are known to one of ordinary skill in the art.

In an embodiment, the PD-1 inhibitor is an antibody disclosed in U.S. Pat. No. 8,354,509 or U.S. Patent Application Publication Nos. 2010/0266617 A1, 2013/0108651 A1, 2013/0109843 A2, the disclosures of which are incorporated by reference herein. In an embodiment, the PD-1 inhibitor is an anti-PD-1 antibody described in U.S. Pat. Nos. 8,287,856, 8,580,247, and 8,168,757 and U.S. Patent Application Publication Nos. 2009/0028857 A1, 2010/0285013 A1, 2013/0022600 A1, and 2011/0008369 A1, the teachings of which are hereby incorporated by reference. In another embodiment, the PD-1 inhibitor is an anti-PD-1 antibody disclosed in U.S. Pat. No. 8,735,553 B1, the disclosure of which is incorporated herein by reference. In an embodiment, the PD-1 inhibitor is pidilizumab, also known as CT-011, which is described in U.S. Pat. No. 8,686,119, the disclosure of which is incorporated by reference herein.

In an embodiment, the PD-1 inhibitor may be a small molecule or a peptide, or a peptide derivative, such as those described in U.S. Pat. Nos. 8,907,053; 9,096,642; and 9,044,442 and U.S. Patent Application Publication No. US 2015/0087581; 1,2,4-oxadiazole compounds and derivatives such as those described in U.S. Patent Application Publication No. 2015/0073024; cyclic peptidomimetic compounds and derivatives such as those described in U.S. Patent Application Publication No. US 2015/0073042; cyclic compounds and derivatives such as those described in U.S. Patent Application Publication No. US 2015/0125491; 1,3,4-oxadiazole and 1,3,4-thiadiazole compounds and derivatives such as those described in International Patent Application Publication No. WO 2015/033301; peptide-based compounds and derivatives such as those described in International Patent Application Publication Nos. WO 2015/036927 and WO 2015/04490, or a macrocyclic peptide-based compounds and derivatives such as those described in U.S. Patent Application Publication No. US 2014/0294898; the disclosures of each of which are hereby incorporated by reference in their entireties.

In an embodiment, the PD-L1 or PD-L2 inhibitor may be any PD-L1 or PD-L2 inhibitor, antagonist, or blocker known in the art. In particular, it is one of the PD-L1 or PD-L2 inhibitors, antagonist, or blockers described in more detail in the following paragraphs. The terms “inhibitor.” “antagonist,” and “blocker” are used interchangeably herein in reference to PD-L1 and PD-L2 inhibitors. For avoidance of doubt, references herein to a PD-L1 or PD-L2 inhibitor that is an antibody may refer to a compound or antigen-binding fragments, variants, conjugates, or biosimilars thereof. For avoidance of doubt, references herein to a PD-L1 or PD-L2 inhibitor may refer to a compound or a pharmaceutically acceptable salt, ester, solvate, hydrate, cocrystal, or prodrug thereof.

In some embodiments, the compositions, processes and methods described herein include a PD-L1 or PD-L2 inhibitor. In some embodiments, the PD-L1 or PD-L2 inhibitor is a small molecule. In a preferred embodiment, the PD-L1 or PD-L2 inhibitor is an antibody (i.e., an anti-PD-1 antibody), a fragment thereof, including Fab fragments, or a single-chain variable fragment (scFv) thereof. In some embodiments the PD-L1 or PD-L2 inhibitor is a polyclonal antibody. In a preferred embodiment, the PD-L1 or PD-L2 inhibitor is a monoclonal antibody. In some embodiments, the PD-L1 or PD-L2 inhibitor competes for binding with PD-L1 or PD-L2, and/or binds to an epitope on PD-L1 or PD-L2. In an embodiment, the antibody competes for binding with PD-L1 or PD-L2, and/or binds to an epitope on PD-L1 or PD-L2.

In some embodiments, the PD-L1 inhibitors provided herein are selective for PD-L1, in that the compounds bind or interact with PD-L1 at substantially lower concentrations than they bind or interact with other receptors, including the PD-L2 receptor. In certain embodiments, the compounds bind to the PD-L1 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L2 receptor.

In some embodiments, the PD-L2 inhibitors provided herein are selective for PD-L2, in that the compounds bind or interact with PD-L2 at substantially lower concentrations than they bind or interact with other receptors, including the PD-L1 receptor. In certain embodiments, the compounds bind to the PD-L2 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L1 receptor.

Without being bound by any theory, it is believed that tumor cells express PD-L1, and that T cells express PD-1. However, PD-L1 expression by tumor cells is not required for efficacy of PD-1 or PD-L1 inhibitors or blockers. In an embodiment, the tumor cells express PD-L1. In another embodiment, the tumor cells do not express PD-L1. In some embodiments, the methods can include a combination of a PD-1 and a PD-L1 antibody, such as those described herein, in combination with a TIL. The administration of a combination of a PD-1 and a PD-L1 antibody and a TIL may be simultaneous or sequential.

In some embodiments, the PD-L1 and/or PD-L2 inhibitor is one that binds human PD-L1 and/or PD-L2 with a KD of about 100 pM or lower, binds human PD-L1 and/or PD-L2 with a KD of about 90 pM or lower, binds human PD-L1 and/or PD-L2 with a KD of about 80 pM or lower, binds human PD-L1 and/or PD-L2 with a KD of about 70 pM or lower, binds human PD-L1 and/or PD-L2 with a KD of about 60 pM or lower, a KD of about 50 pM or lower, binds human PD-L1 and/or PD-L2 with a KD of about 40 pM or lower, or binds human PD-L1 and/or PD-L2 with a KD of about 30 pM or lower,

In some embodiments, the PD-L1 and/or PD-L2 inhibitor is one that binds to human PD-L1 and/or PD-L2 with a k_{assoc} of about 7.5×10⁵ l/M·s or faster, binds to human PD-L1 and/or PD-L2 with a k_{assoc} of about 8×10⁵ l/M·s or faster, binds to human PD-L1 and/or PD-L2 with a k_{assoc} of about 8.5×10⁵ l/M·s or faster, binds to human PD-L1 and/or PD-L2 with a k_{assoc} of about 9×10⁵ l/M·s or faster, binds to human PD-L1 and/or PD-L2 with a k_{assoc} of about 9.5×10⁵ l/M·s and/or faster, or binds to human PD-L1 and/or PD-L2 with a k_{assoc} of about 1×10⁶ l/M·s or faster.

In some embodiments, the PD-L1 and/or PD-L2 inhibitor is one that binds to human PD-L1 or PD-L2 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10⁻⁵ l/s or slower, binds to human PD-1 with a k_{dissoc} of about 2.5×10⁻⁵ l/s or slower, binds to human PD-1 with a k_{dissoc} of about 2.6×10⁻⁵ l/s or slower, binds to human PD-L1 or PD-L2 with a k_{dissoc} of about 2.7×10⁻⁵ l/s or slower, or binds to human PD-L1 or PD-L2 with a k_{dissoc} of about 3×10⁻⁵ l/s or slower.

In some embodiments, the PD-L1 and/or PD-L2 inhibitor is one that blocks or inhibits binding of human PD-L1 or human PD-L2 to human PD-1 with an IC50 of about 10 nM or lower; blocks or inhibits binding of human PD-L1 or human PD-L2 to human PD-1 with an IC50 of about 9 nM or lower; blocks or inhibits binding of human PD-L1 or human PD-L2 to human PD-1 with an IC50 of about 8 nM or lower; blocks or inhibits binding of human PD-L1 or human PD-L2 to human PD-1 with an IC50 of about 7 nM or lower; blocks or inhibits binding of human PD-L1 or human PD-L2 to human PD-1 with an IC50 of about 6 nM or lower; blocks or inhibits binding of human PD-L1 or human PD-L2 to human PD-1 with an IC50 of about 5 nM or lower; blocks or inhibits binding of human PD-L1 or human PD-L2 to human PD-1 with an IC50 of about 4 nM or lower; blocks or inhibits binding of human PD-L1 or human PD-L2 to human PD-1 with an IC50 of about 3 nM or lower; blocks or inhibits binding of human PD-L1 or human PD-L2 to human PD-1 with an IC50 of about 2 nM or lower; or blocks human PD-1, or blocks binding of human PD-L1 or human PD-L2 to human PD-1 with an IC50 of about 1 nM or lower.

In an embodiment, the PD-L1 inhibitor is durvalumab, also known as MEDI4736 (which is commercially available from Medimmune, LLC, Gaithersburg, Maryland, a subsidiary of AstraZeneca plc.), or antigen-binding fragments, conjugates, or variants thereof. In an embodiment, the PD-L1 inhibitor is an antibody disclosed in U.S. Pat. No. 8,779,108 or U.S. Patent Application Publication No. 2013/0034559, the disclosures of which are incorporated by reference herein. The clinical efficacy of durvalumab has been described in Page, et al., Ann. Rev. Med., 2014, 65, 185-202; Brahmer, et al., J. Clin. Oncol. 2014, 32, 5s (supplement, abstract 8021); and McDermott, et al., Cancer Treatment Rev., 2014, 40, 1056-64. The preparation and properties of durvalumab are described in U.S. Pat. No. 8,779,108, the disclosure of which is incorporated by reference herein. The amino acid sequences of durvalumab are set forth in Table 20. The durvalumab monoclonal antibody includes disulfide linkages at 22-96, 22″-96″, 23′-89′, 23′″-89′″, 135′-195′, 135′″-195′″, 148-204, 148″-204″, 215′-224, 215′″-224″, 230-230″, 233-233″, 265-325, 265″-325″, 371-429, and 371″-429′; and N-glycosylation sites at Asn-301 and Asn-301″.

In an embodiment, a PD-L1 inhibitor comprises a heavy chain given by SEQ ID NO:178 and a light chain given by SEQ ID NO:179. In an embodiment, a PD-L1 inhibitor comprises heavy and light chains having the sequences shown in SEQ ID NO:178 and SEQ ID NO:179, respectively, or antigen binding fragments, Fab fragments, single-chain variable fragments (scFv), variants, or conjugates thereof. In an embodiment, a PD-L1 inhibitor comprises heavy and light chains that are each at least 99% identical to the sequences shown in SEQ ID NO:178 and SEQ ID NO:179, respectively. In an embodiment, a PD-L1 inhibitor comprises heavy and light chains that are each at least 98% identical to the sequences shown in SEQ ID NO:178 and SEQ ID NO:179, respectively. In an embodiment, a PD-L1 inhibitor comprises heavy and light chains that are each at least 97% identical to the sequences shown in SEQ ID NO:178 and SEQ ID NO:179, respectively. In an embodiment, a PD-L1 inhibitor comprises heavy and light chains that are each at least 96% identical to the sequences shown in SEQ ID NO:178 and SEQ ID NO:179, respectively. In an embodiment, a PD-L1 inhibitor comprises heavy and light chains that are each at least 95% identical to the sequences shown in SEQ ID NO:178 and SEQ ID NO:179, respectively.

In an embodiment, the PD-L1 inhibitor comprises the heavy and light chain CDRs or variable regions (VRs) of durvalumab. In an embodiment, the PD-L1 inhibitor heavy chain variable region (V_{H}) comprises the sequence shown in SEQ ID NO:180, and the PD-L1 inhibitor light chain variable region (V_{L}) comprises the sequence shown in SEQ ID NO:181, or conservative amino acid substitutions thereof. In an embodiment, a PD-L1 inhibitor comprises V_{H} and V_{L} regions that are each at least 99% identical to the sequences shown in SEQ ID NO:180 and SEQ ID NO:181, respectively. In an embodiment, a PD-L1 inhibitor comprises V_{H} and V_{L} regions that are each at least 98% identical to the sequences shown in SEQ ID NO:180 and SEQ ID NO:181, respectively. In an embodiment, a PD-L1 inhibitor comprises V_{H} and V_{L} regions that are each at least 97% identical to the sequences shown in SEQ ID NO:180 and SEQ ID NO:181, respectively. In an embodiment, a PD-L1 inhibitor comprises V_{H} and V_{L} regions that are each at least 96% identical to the sequences shown in SEQ ID NO:180 and SEQ ID NO:181, respectively. In an embodiment, a PD-L1 inhibitor comprises V_{H} and V_{L} regions that are each at least 95% identical to the sequences shown in SEQ ID NO:180 and SEQ ID NO:181, respectively.

In an embodiment, a PD-L1 inhibitor comprises heavy chain CDR1, CDR2 and CDR3 domains having the sequences set forth in SEQ ID NO:182, SEQ ID NO:183, and SEQ ID NO:184, respectively, or conservative amino acid substitutions thereof, and light chain CDR1, CDR2 and CDR3 domains having the sequences set forth in SEQ ID NO:185, SEQ ID NO:186, and SEQ ID NO:187, respectively, or conservative amino acid substitutions thereof. In an embodiment, the antibody competes for binding with, and/or binds to the same epitope on PD-L1 as any of the aforementioned antibodies.

In an embodiment, the PD-L1 inhibitor is an anti-PD-L1 biosimilar monoclonal antibody approved by drug regulatory authorities with reference to durvalumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is durvalumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is durvalumab. The ant-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab.

TABLE 20
Amino acid sequences for PD-L1 inhibitors related to durvalumab.

| Identifier | Sequence (One-Letter Amino Acid Symbols) | |
|---|---|---|
| SEQ ID NO: 178 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| heavy chain | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPEFEG | 240 |
| | GPSVFLFPPK PKDTLMISRT PEVTCVVVDV SHEDPEVKEN WYVDGVEVHN AKTKPREEQY | 300 |
| | NSTYRVVSVL TVLHQDWING KEYKCKVSNK ALPASIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 179 | EVQLVESGGG LVQPGGSLRL SCAASGFTFS RYWMSWVRQA PGKGLEWVAN EIVLTQSPGT | 60 |
| durvalumab | LSLSPGERAT LSCRASQRVS SSYLAWYQQK PGQAPRLLIY DASSRATGIP DRFSGSGSGT | 120 |
| light chain | DETLTISRLE PEDFAVYYCQ QYGSLPWTFG QGTKVEIKRT VAAPSVFIFP PSDEQLKSGT | 180 |
| | ASVVCLLNNF YPREAKVQWK VDNALQSGNS QESVTEQDSK DSTYSLSSTL TLSKADYEKH | 240 |
| | KVYACEVTHQ GLSSPVTKSF NRGEC | 265 |
| SEQ ID NO: 180 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| variable | S | 121 |
| heavy chain | | |
| SEQ ID NO: 181 | EIVLTQSPGT LSLSPGERAT LSCRASQRVS SSYLAWYQQK PGQAPRLLIY DASSRATGIP | 60 |
| durvalumab | DRESGSGSGT DFTLTISRLE PEDFAVYYCQ QYGSLPWTFG QGTKVEIK | 108 |
| variable | | |
| light chain | | |
| SEQ ID NO: 182 | RYWMS | 5 |
| durvalumab | | |
| heavy chain | | |
| CDR1 | | |
| SEQ ID NO: 183 | NIKQDGSEKY YVDSVKG | 17 |
| durvalumab | | |
| heavy chain | | |
| CDR2 | | |
| SEQ ID NO: 184 | EGGWFGELAF DY | 12 |
| durvalumab | | |
| heavy chain | | |
| CDR3 | | |
| SEQ ID NO: 185 | RASQRVSSSY LA | 12 |
| durvalumab | | |
| light chain | | |
| CDR1 | | |
| SEQ ID NO: 186 | DASSRAT | 7 |
| durvalumab | | |
| light chain | | |
| CDR2 | | |
| SEQ ID NO: 187 | QQYGSLPWT | 9 |
| durvalumab | | |
| light chain | | |
| CDR3 | | |

In an embodiment, the PD-L1 inhibitor is avelumab, also known as MSB0010718C (commercially available from Merck KGaA/EMD Serono), or antigen-binding fragments, conjugates, or variants thereof. The preparation and properties of avelumab are described in U.S. Patent Application Publication No. US 2014/0341917 A1, the disclosure of which is specifically incorporated by reference herein. The amino acid sequences of avelumab are set forth in Table 21. Avelumab has intra-heavy chain disulfide linkages (C23-C104) at 22-96, 147-203, 264-324, 370-428, 22″-96″, 147″-203″, 264″-324″, and 370″-428″; intra-light chain disulfide linkages (C23-C104) at 22′-90′, 138′-197′, 22′″-90′″, and 138′″-197′″; intra-heavy-light chain disulfide linkages (h 5-CL 126) at 223-215′ and 223″-215′″; intra-heavy-heavy chain disulfide linkages (h 11, h 14) at 229-229″ and 232-232″; N-glycosylation sites (H CH2 N84.4) at 300, 300″; fucosylated complex bi-antennary CHO-type glycans; and H CHS K2 C-terminal lysine clipping at 450 and 450′.

In an embodiment, a PD-L1 inhibitor comprises a heavy chain given by SEQ ID NO:188 and a light chain given by SEQ ID NO:189. In an embodiment, a PD-L1 inhibitor comprises heavy and light chains having the sequences shown in SEQ ID NO:188 and SEQ ID NO:189, respectively, or antigen binding fragments, Fab fragments, single-chain variable fragments (scFv), variants, or conjugates thereof. In an embodiment, a PD-L1 inhibitor comprises heavy and light chains that are each at least 99% identical to the sequences shown in SEQ ID NO:188 and SEQ ID NO:189, respectively. In an embodiment, a PD-L1 inhibitor comprises heavy and light chains that are each at least 98% identical to the sequences shown in SEQ ID NO:188 and SEQ ID NO:189, respectively. In an embodiment, a PD-L1 inhibitor comprises heavy and light chains that are each at least 97% identical to the sequences shown in SEQ ID NO:188 and SEQ ID NO:189, respectively. In an embodiment, a PD-L1 inhibitor comprises heavy and light chains that are each at least 96% identical to the sequences shown in SEQ ID NO:188 and SEQ ID NO:189, respectively. In an embodiment, a PD-L1 inhibitor comprises heavy and light chains that are each at least 95% identical to the sequences shown in SEQ ID NO:188 and SEQ ID NO:189, respectively.

In an embodiment, the PD-L1 inhibitor comprises the heavy and light chain CDRs or variable regions (VRs) of avelumab. In an embodiment, the PD-L1 inhibitor heavy chain variable region (VH) comprises the sequence shown in SEQ ID NO:190, and the PD-L1 inhibitor light chain variable region (VL) comprises the sequence shown in SEQ ID NO:191, or conservative amino acid substitutions thereof. In an embodiment, a PD-L1 inhibitor comprises V_{H} and V_{L} regions that are each at least 99% identical to the sequences shown in SEQ ID NO:190 and SEQ ID NO:191, respectively. In an embodiment, a PD-L1 inhibitor comprises V_{H} and V_{L} regions that are each at least 98% identical to the sequences shown in SEQ ID NO:190 and SEQ ID NO:191, respectively. In an embodiment, a PD-L1 inhibitor comprises V_{H} and V_{L} regions that are each at least 97% identical to the sequences shown in SEQ ID NO:190 and SEQ ID NO:191, respectively. In an embodiment, a PD-L1 inhibitor comprises V_{H} and V_{L} regions that are each at least 96% identical to the sequences shown in SEQ ID NO:190 and SEQ ID NO:191, respectively. In an embodiment, a PD-L1 inhibitor comprises V_{H} and V_{L} regions that are each at least 95% identical to the sequences shown in SEQ ID NO:190 and SEQ ID NO:191, respectively.

In an embodiment, a PD-L1 inhibitor comprises heavy chain CDR1, CDR2 and CDR3 domains having the sequences set forth in SEQ ID NO:192, SEQ ID NO:193, and SEQ ID NO:194, respectively, or conservative amino acid substitutions thereof, and light chain CDR1, CDR2 and CDR3 domains having the sequences set forth in SEQ ID NO:195, SEQ ID NO:196, and SEQ ID NO:197, respectively, or conservative amino acid substitutions thereof. In an embodiment, the antibody competes for binding with, and/or binds to the same epitope on PD-L1 as any of the aforementioned antibodies.

In an embodiment, the PD-L1 inhibitor is an anti-PD-L1 biosimilar monoclonal antibody approved by drug regulatory authorities with reference to avelumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is avelumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is avelumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab.

TABLE 21
Amino acid sequences for PD-L1 inhibitors related to avelumab.

| Identifier | Sequence (One-Letter Amino Acid Symbols) | |
|---|---|---|
| SEQ ID NO: 188 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| heavy chain | ASTKGPSVFP LAPSSKSTSG GTAALGCLVK DYFPEPVTVS WNSGALTSGV HTFPAVLQSS | 180 |
| | GLYSLSSVVT VPSSSLGTQT YICNVNEKPS NTKVDKKVEP KSCDKTHTCP PCPAPELLGG | 240 |
| | PSVFLFPPKP KDTLMISRTP EVTCVVVDVS HEDPEVKENW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 189 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYESSSTRV FGTGTKVTVL GQPKANPTVT | 120 |
| light chain | LFPPSSEELQ ANKATLVCLI SDFYPGAVTV AWKADGSPVK AGVETTKPSK QSNNKYAASS | 180 |
| | YLSLTPEQWK SHRSYSCQVT HEGSTVEKTV APTECS | 216 |
| SEQ ID NO: 190 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| variable | | |
| heavy chain | | |
| SEQ ID NO: 191 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYESSSTRV FGTGTKVTVL | 110 |
| variable | | |
| light chain | | |
| SEQ ID NO: 192 | SYIMM | 5 |
| avelumab | | |
| heavy chain | | |
| CDR1 | | |
| SEQ ID NO: 193 | SIYPSGGITF YADTVKG | 17 |
| avelumab | | |
| heavy chain | | |
| CDR2 | | |
| SEQ ID NO: 194 | IKLGTVTTVD Y | 11 |
| avelumab | | |
| heavy chain | | |
| CDR3 | | |
| SEQ ID NO: 195 | TGTSSDVGGY NYVS | 14 |
| avelumab | | |
| light chain | | |
| CDR1 | | |
| SEQ ID NO: 196 | DVSNRPS | 7 |
| avelumab | | |
| light chain | | |
| CDR2 | | |
| SEQ ID NO: 197 | SSYTSSSTRV | 10 |
| avelumab | | |
| light chain | | |
| CDR3 | | |

In an embodiment, the PD-L1 inhibitor is atezolizumab, also known as MPDL3280A or RG7446 (commercially available as TECENTRIQ from Genentech, Inc., a subsidiary of Roche Holding AG, Basel, Switzerland), or antigen-binding fragments, conjugates, or variants thereof. In an embodiment, the PD-L1 inhibitor is an antibody disclosed in U.S. Pat. No. 8,217,149, the disclosure of which is specifically incorporated by reference herein. In an embodiment, the PD-L1 inhibitor is an antibody disclosed in U.S. Patent Application Publication Nos. 2010/0203056 A1, 2013/0045200 A1, 2013/0045201 A1, 2013/0045202 A1, or 2014/0065135 A1, the disclosures of which are specifically incorporated by reference herein. The preparation and properties of atezolizumab are described in U.S. Pat. No. 8,217,149, the disclosure of which is incorporated by reference herein. The amino acid sequences of atezolizumab are set forth in Table 22. Atezolizumab has intra-heavy chain disulfide linkages (C23-C104) at 22-96, 145-201, 262-322, 368-426, 22″-96″, 145″-201″, 262″-322″, and 368″-426″; intra-light chain disulfide linkages (C23-C104) at 23′-88′, 134′-194′, 23′″-88′″, and 134′″-194′″; intra-heavy-light chain disulfide linkages (h 5-CL 126) at 221-214′ and 221″-214′″; intra-heavy-heavy chain disulfide linkages (h 11, h 14) at 227-227″ and 230-230″; and N-glycosylation sites (H CH2 N84.4>A) at 298 and 298′.

In an embodiment, a PD-L1 inhibitor comprises a heavy chain given by SEQ ID NO:198 and a light chain given by SEQ ID NO:199. In an embodiment, a PD-L1 inhibitor comprises heavy and light chains having the sequences shown in SEQ ID NO:198 and SEQ ID NO:199, respectively, or antigen binding fragments, Fab fragments, single-chain variable fragments (scFv), variants, or conjugates thereof. In an embodiment, a PD-L1 inhibitor comprises heavy and light chains that are each at least 99% identical to the sequences shown in SEQ ID NO:198 and SEQ ID NO:199, respectively. In an embodiment, a PD-L1 inhibitor comprises heavy and light chains that are each at least 98% identical to the sequences shown in SEQ ID NO:198 and SEQ ID NO:199, respectively. In an embodiment, a PD-L1 inhibitor comprises heavy and light chains that are each at least 97% identical to the sequences shown in SEQ ID NO:198 and SEQ ID NO:199, respectively. In an embodiment, a PD-L1 inhibitor comprises heavy and light chains that are each at least 96% identical to the sequences shown in SEQ ID NO 198 and SEQ ID NO 199 respectively. In an embodiment a PD-L1 inhibitor comprises heavy and light chains that are each at least 95% identical to the sequences shown in SEQ ID NO:198 and SEQ ID NO:199, respectively.

In an embodiment, the PD-L1 inhibitor comprises the heavy and light chain CDRs or variable regions (VRs) of atezolizumab. In an embodiment, the PD-L1 inhibitor heavy chain variable region (V_{H}) comprises the sequence shown in SEQ ID NO:200, and the PD-L1 inhibitor light chain variable region (V_{L}) comprises the sequence shown in SEQ ID NO:201, or conservative amino acid substitutions thereof. In an embodiment, a PD-L1 inhibitor comprises V_{H} and V_{L} regions that are each at least 99% identical to the sequences shown in SEQ ID NO:200 and SEQ ID NO:201, respectively. In an embodiment, a PD-L1 inhibitor comprises V_{H} and V_{L} regions that are each at least 98% identical to the sequences shown in SEQ ID NO:200 and SEQ ID NO:201, respectively. In an embodiment, a PD-L1 inhibitor comprises V_{H} and V_{L} regions that are each at least 97% identical to the sequences shown in SEQ ID NO:200 and SEQ ID NO:201, respectively. In an embodiment, a PD-L1 inhibitor comprises V_{H} and V_{L} regions that are each at least 96% identical to the sequences shown in SEQ ID NO:200 and SEQ ID NO:201, respectively. In an embodiment, a PD-L1 inhibitor comprises V_{H} and V_{L} regions that are each at least 95% identical to the sequences shown in SEQ ID NO:200 and SEQ ID NO:201, respectively.

In an embodiment, a PD-L1 inhibitor comprises heavy chain CDR1, CDR2 and CDR3 domains having the sequences set forth in SEQ ID NO:202, SEQ ID NO:203, and SEQ ID NO:204, respectively, or conservative amino acid substitutions thereof, and light chain CDR1, CDR2 and CDR3 domains having the sequences set forth in SEQ ID NO:205, SEQ ID NO:206, and SEQ ID NO:207, respectively, or conservative amino acid substitutions thereof. In an embodiment, the antibody competes for binding with, and/or binds to the same epitope on PD-L1 as any of the aforementioned antibodies.

In an embodiment, the anti-PD-L1 antibody is an anti-PD-L1 biosimilar monoclonal antibody approved by drug regulatory authorities with reference to atezolizumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is atezolizumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is atezolizumab. The anti-PD-L1 antibody may be authorized by a drug regulatory authority such as the U.S. FDA ad/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab.

TABLE 22
Amino acid sequences for PD-L1 inhibitors related to atezolizumab.

| Identifier | Sequence (One-Letter Amino Acid Symbols) | |
|---|---|---|
| SEQ ID NO: 198 | EVQLVESGGG LVQPGGSLRL SCAASGFTFS DSWEHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKKVEPKS CDKTHTCPPC PAPELLGGPS | 240 |
| | VFLFPPKPKD TLMISRTPEV TCVVVDVSHE DPEVKFNWYV DGVEVHNAKT KPREEQYAST | 300 |
| | YRVVSVLTVL HQDWLNGKEY KCKVSNKALP APIEKTISKA KGQPREPQVY TLPPSREEMT | 360 |
| | KNQVSLTCLV KGFYPSDIAV EWESNGQPEN NYKSTPPVLD SDGSFFLYSK ITVDKSRWQQ | 420 |
| | GNVFSCSVMH EALHNHYTQK SLSLSPGK | 448 |
| SEQ ID NO: 199 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSEN RGEC | 214 |
| SEQ ID NO: 200 | EVQLVESGGG LVQPGGSLRL SCAASGFTFS DSWEHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSA | 118 |
| variable | | |
| heavy chain | | |
| SEQ ID NO: 201 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKR | 108 |
| variable | | |
| light chain | | |
| SEQ ID NO: 202 | GFTFSDSWIH | 10 |
| atezolizumab | | |
| heavy chain | | |
| CDR1 | | |
| SEQ ID NO: 203 | AWISPYGGST YYADSVKG | 18 |
| atezolizumab | | |
| heavy chain | | |
| CDR2 | | |
| SEQ ID NO: 204 | RHWPGGFDY | 9 |
| atezolizumab | | |
| heavy chain | | |
| CDR3 | | |
| SEQ ID NO: 205 | RASQDVSTAV A | 11 |
| atezolizumab | | |
| light chain | | |
| CDR1 | | |
| SEQ ID NO: 206 | SASFLYS | 7 |
| atezolizumab | | |
| light chain | | |
| CDR2 | | |
| SEQ ID NO: 207 | QQYLYHPAT | 9 |
| atezolizumab | | |
| light chain | | |
| CDR3 | | |

In an embodiment, the PD-L1 inhibitor or anti-PD-L1 antibody is retifanlimab, or a fragment, variant, conjugate, or biosimilar thereof, which is available from Incyte, Inc.

In an embodiment, PD-L1 inhibitors include those antibodies described in U.S. Patent Application Publication No. US 2014/0341917 A1, the disclosure of which is incorporated by reference herein. In another embodiment, antibodies that compete with any of these antibodies for binding to PD-L1 are also included. In an embodiment, the anti-PD-L1 antibody is MDX-1105, also known as BMS-935559, which is disclosed in U.S. Pat. No. 7,943,743, the disclosures of which are incorporated by reference herein. In an embodiment, the anti-PD-L1 antibody is selected from the anti-PD-L1 antibodies disclosed in U.S. Pat. No. 7,943,743, which are incorporated by reference herein.

In an embodiment, the PD-L1 inhibitor is a commercially-available monoclonal antibody, such as INVIVOMAB anti-m-PD-L1 clone 10F.9G2 (Catalog #BE0101, Bio X Cell, Inc., West Lebanon, NH, USA). In an embodiment, the anti-PD-L1 antibody is a commercially-available monoclonal antibody, such as AFFYMETRIX EBIOSCIENCE (MIH1). A number of commercially-available anti-PD-L1 antibodies are known to one of ordinary skill in the art.

In an embodiment, the PD-L2 inhibitor is a commercially-available monoclonal antibody, such as BIOLEGEND 24F.10C12 Mouse IgG2a, K isotype (catalog #329602 Biolegend, Inc., San Diego, CA), SIGMA anti-PD-L2 antibody (catalog #SAB3500395, Sigma-Aldrich Co., St. Louis, MO), or other commercially-available anti-PD-L2 antibodies known to one of ordinary skill in the art.

In some embodiments, the present invention includes a method of treating a patient with a cancer comprising the steps of administering a TIL regimen, wherein the TIL regimen includes a TIL product genetically modified to express a CCR, further comprising the step of administering either a PD-1 inhibitor or a PD-L1 inhibitor. In some embodiments, the present invention includes a composition comprising (i) a TIL product genetically modified to express a CCR and (ii) either a PD-1 inhibitor or a PD-L1 inhibitor. In some embodiments, the present invention includes a kit comprising (i) a TIL product genetically modified to express a CCR and (ii) either a PD-1 inhibitor or a PD-L1 inhibitor.

3. Combinations with CTLA-4 Inhibitors

In some embodiments, the TIL therapy provided to patients with cancer may include treatment with therapeutic populations of TILs alone or may include a combination treatment including TILs and one or more CTLA-4 inhibitors.

Cytotoxic T lymphocyte antigen 4 (CTLA-4) is a member of the immunoglobulin superfamily and is expressed on the surface of helper T cells. CTLA-4 is a negative regulator of CD28-dependent T cell activation and acts as a checkpoint for adaptive immune responses. Similar to the T cell costimulatory protein CD28, the CTLA-4 binding antigen presents CD80 and CD86 on the cells. CTLA-4 delivers a suppressor signal to T cells, while CD28 delivers a stimulus signal. Human antibodies against human CTLA-4 have been described as immunostimulatory modulators in many disease conditions, such as treating or preventing viral and bacterial infections and for treating cancer (WO 01/14424 and WO 00/37504). A number of fully human anti-human CTLA-4 monoclonal antibodies (mAbs) have been studied in clinical trials for the treatment of various types of solid tumors, including, but not limited to, ipilimumab (MDX-010) and tremelimumab (CP-675,206).

In some embodiments, a CTLA-4 inhibitor may be any CTLA-4 inhibitor or CTLA-4 blocker known in the art. In particular, it is one of the CTLA-4 inhibitors or blockers described in more detail in the following paragraphs. The terms “inhibitor,” “antagonist,” and “blocker” are used interchangeably herein in reference to CTLA-4 inhibitors. For avoidance of doubt, references herein to a CTLA-4 inhibitor that is an antibody may refer to a compound or antigen-binding fragments, variants, conjugates, or biosimilars thereof. For avoidance of doubt, references herein to a CTLA-4 inhibitor may also refer to a small molecule compound or a pharmaceutically acceptable salt, ester, solvate, hydrate, cocrystal, or prodrug thereof.

Suitable CTLA-4 inhibitors for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1, the disclosures of each of which are incorporated herein by reference. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014, the disclosures of each of which are incorporated herein by reference. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281, the disclosures of each of which are incorporated herein by reference.

Additional CTLA-4 inhibitors include, but are not limited to, the following: any inhibitor that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD80, CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; adnectins directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other CTLA-4 inhibitors.

In some embodiments a CTLA-4 inhibitor binds to CTLA-4 with a K_{d} of about 10⁻⁶ M or less, 10⁻⁷M or less, 10⁻⁸ M or less, 10⁻⁹ M or less, 10⁻¹⁰ M or less, 10⁻¹¹ M or less, 10⁻¹² M or less, e.g., between 10⁻¹³ M and 10⁻¹⁶ M, or within any range having any two of the afore-mentioned values as endpoints. In some embodiments a CTLA-4 inhibitor binds to CTLA-4 with a Kd of no more than 10-fold that of ipilimumab, when compared using the same assay. In some embodiments a CTLA-4 inhibitor binds to CTLA-4 with a Kd of about the same as, or less (e.g., up to 10-fold lower, or up to 100-fold lower) than that of ipilimumab, when compared using the same assay. In some embodiments, the IC50 values for inhibition by a CTLA-4 inhibitor of CTLA-4 binding to CD80 or CD86 is no more than 10-fold greater than that of ipilimumab-mediated inhibition of CTLA-4 binding to CD80 or CD86, respectively, when compared using the same assay. In some embodiments, the IC50 values for inhibition by a CTLA-4 inhibitor of CTLA-4 binding to CD80 or CD86 is about the same or less (e.g., up to 10-fold lower, or up to 100-fold lower) than that of ipilimumab-mediated inhibition of CTLA-4 binding to CD80 or CD86, respectively, when compared using the same assay.

In some embodiments a CTLA-4 inhibitor is used in an amount sufficient to inhibit expression and/or decrease biological activity of CTLA-4 by at least 20%, 30%, 40%, 50%, 60%, 70%, 80%, 90%, 95%, or 100% relative to a suitable control, e.g., between 50% and 75%, 75% and 90%, or 90% and 100%. In some embodiments a CTLA-4 pathway inhibitor is used in an amount sufficient to decrease the biological activity of CTLA-4 by reducing binding of CTLA-4 to CD80, CD86, or both by at least 20%, 30%, 40%, 50%, 60%, 70%, 80%, 90%, 95%, or 100% relative to a suitable control, e.g., between 50% and 75%, 75% and 90%, or 90% and 100% relative to a suitable control. A suitable control in the context of assessing or quantifying the effect of an agent of interest is typically a comparable biological system (e.g., cells or a subject) that has not been exposed to or treated with the agent of interest, e.g., CTLA-4 pathway inhibitor (or has been exposed to or treated with a negligible amount). In some embodiments a biological system may serve as its own control (e.g., the biological system may be assessed before exposure to or treatment with the agent and compared with the state after exposure or treatment has started or finished. In some embodiments a historical control may be used.

In an embodiment, the CTLA-4 inhibitor is ipilimumab (commercially available as Yervoy from Bristol-Myers Squibb Co.), or biosimilars, antigen-binding fragments, conjugates, or variants thereof. As is known in the art, ipilimumab refers to an anti-CTLA-4 antibody, a fully human IgG 1κ antibody derived from a transgenic mouse with human genes encoding heavy and light chains to generate a functional human repertoire. is there. Ipilimumab can also be referred to by its CAS Registry Number 477202-00-9, and in PCT Publication Number WO 01/14424, which is incorporated herein by reference in its entirety for all purposes. It is disclosed as antibody 10DI. Specifically, ipilimumab contains a light chain variable region and a heavy chain variable region (having a light chain variable region comprising SEQ ID NO:211 and having a heavy chain variable region comprising SEQ ID NO:210). A pharmaceutical composition of ipilimumab includes all pharmaceutically acceptable compositions containing ipilimumab and one or more diluents, vehicles, or excipients. An example of a pharmaceutical composition containing ipilimumab is described in International Patent Application Publication No. WO 2007/67959. Ipilimumab can be administered intravenously (IV).

In an embodiment, a CTLA-4 inhibitor comprises a heavy chain given by SEQ ID NO:208 and a light chain given by SEQ ID NO:209. In an embodiment, a CTLA-4 inhibitor comprises heavy and light chains having the sequences shown in SEQ ID NO:208 and SEQ ID NO:209, respectively, or antigen binding fragments, Fab fragments, single-chain variable fragments (scFv), variants, or conjugates thereof. In an embodiment, a CTLA-4 inhibitor comprises heavy and light chains that are each at least 99% identical to the sequences shown in SEQ ID NO:208 and SEQ ID NO:209, respectively. In an embodiment, a CTLA-4 inhibitor comprises heavy and light chains that are each at least 98% identical to the sequences shown in SEQ ID NO:208 and SEQ ID NO:209, respectively. In an embodiment, a CTLA-4 inhibitor comprises heavy and light chains that are each at least 97% identical to the sequences shown in SEQ ID NO:208 and SEQ ID NO:209, respectively. In an embodiment, a CTLA-4 inhibitor comprises heavy and light chains that are each at least 96% identical to the sequences shown in SEQ ID NO:208 and SEQ ID NO:209, respectively. In an embodiment, a CTLA-4 inhibitor comprises heavy and light chains that are each at least 95% identical to the sequences shown in SEQ ID NO:208 and SEQ ID NO:209, respectively.

In an embodiment, the CTLA-4 inhibitor comprises the heavy and light chain CDRs or variable regions (VRs) of ipilimumab. In an embodiment, the CTLA-4 inhibitor heavy chain variable region (V_{H}) comprises the sequence shown in SEQ ID NO:210, and the CTLA-4 inhibitor light chain variable region (V_{L}) comprises the sequence shown in SEQ ID NO:211, or conservative amino acid substitutions thereof. In an embodiment, a CTLA-4 inhibitor comprises V_{H} and V_{L} regions that are each at least 99% identical to the sequences shown in SEQ ID NO:210 and SEQ ID NO:211, respectively. In an embodiment, a CTLA-4 inhibitor comprises V_{H} and V_{L} regions that are each at least 98% identical to the sequences shown in SEQ ID NO:210 and SEQ ID NO:211, respectively. In an embodiment, a CTLA-4 inhibitor comprises V_{H} and V_{L} regions that are each at least 97% identical to the sequences shown in SEQ ID NO:210 and SEQ ID NO:211, respectively. In an embodiment, a CTLA-4 inhibitor comprises V_{H} and V_{L} regions that are each at least 96% identical to the sequences shown in SEQ ID NO:210 and SEQ ID NO:211, respectively. In an embodiment, a CTLA-4 inhibitor comprises V_{H} and V_{L} regions that are each at least 95% identical to the sequences shown in SEQ ID NO:210 and SEQ ID NO:211, respectively.

In an embodiment, a CTLA-4 inhibitor comprises the heavy chain CDR1. CDR2 and CDR3 domains having the sequences set forth in SEQ ID NO:212, SEQ ID NO:213, and SEQ ID NO:214, respectively, or conservative amino acid substitutions thereof, and light chain CDR1, CDR2 and CDR3 domains having the sequences set forth in SEQ ID NO:215, SEQ ID NO:216, and SEQ ID NO:217, respectively, or conservative amino acid substitutions thereof. In an embodiment, the antibody competes for binding with, and/or binds to the same epitope on CTLA-4 as any of the aforementioned antibodies.

In an embodiment, the CTLA-4 inhibitor is a CTLA-4 biosimilar monoclonal antibody approved by drug regulatory authorities with reference to ipilimumab. In an embodiment, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is ipilimumab. In some embodiments, the one or more post-translational modifications are selected from one or more of: glycosylation, oxidation, deamidation, and truncation. The amino acid sequences of ipilimumab are set forth in Table 23. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is ipilimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is ipilimumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is ipilimumab.

TABLE 23
Amino acid sequences for ipilimumab.

| Identifier | Sequence (One-Letter Amino Acid Symbols) | |
|---|---|---|
| SEQ ID NO: 208 | QVQLVESGGG VVQPGRSIRL SCAASGFTFS SYTMHWVRQA PGKGLEWVTF ISYDGNNKYY | 60 |
| ipilimumab | ADSVKGRFTI SRDNSKNTLY LQMNSLRAED TAIYYCARTG WLGPFDYWGQ GTLVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKRVEPKS CDKTH | 225 |
| SEQ ID NO: 209 | EIVLTQSPGT LSLSPGERAT LSCRASQSVG SSYLAWYQQK PGQAPRLLIY GAFSRATGIP | 60 |
| ipilimumab | DRFSGSGSGT DFTLTISRLE PEDFAVYYCQ QYGSSPWTFG QGTKVEIKRT VAAPSVFIFP | 120 |
| light chain | PSDEQLKSGT ASVVCLLNNF YPREAKVQWK VDNALQSGNS QESVTEQDSK DSTYSLSSTL | 180 |
| | TLSKADYEKH KVYACEVTHQ GLSSPVTKSF NRGEC | 215 |
| SEQ ID NO: 210 | QVQLVESGGG VVQPGRSIRL SCAASGFTFS SYTMHWVRQA PGKGLEWVTF ISYDGNNKYY | 60 |
| ipilimumab | ADSVKGRFTI SRDNSKNTLY LQMNSLRAED TAIYYCARTG WLGPFDYWGQ GTLVTVSS | 118 |
| variable heavy | | |
| chain | | |
| SEQ ID NO: 211 | DRFSGSGSGT DFTLTISRLE PEDFAVYYCQ QYGSSPWTFG QGTKVEIK | 60 |
| ipilimumab | EIVLTQSPGT LSLSPGERAT LSCRASQSVG SSYLAWYQQK PGQAPRLLIY GAFSRATGIP | 108 |
| variable light | | |
| chain | | |
| SEQ ID NO: 212 | GFTFSSYT | 8 |
| ipilimumab | | |
| heavy chain | | |
| CDR1 | | |
| SEQ ID NO: 213 | TFISYDGNNK | 10 |
| ipilimumab | | |
| heavy chain | | |
| CDR2 | | |
| SEQ ID NO: 214 | ARTGWLGPFD Y | 11 |
| ipilimumab | | |
| heavy chain | | |
| CDR3 | | |
| SEQ ID NO: 215 | QSVGSSY | 7 |
| ipilimumab | | |
| light chain | | |
| CDR1 | | |
| SEQ ID NO: 216 | GAF | 3 |
| ipilimumab | | |
| light chain | | |
| CDR2 | | |
| SEQ ID NO: 217 | QQYGSSPWT | 9 |
| ipilimumab | | |
| light chain | | |
| CDR3 | | |

In some embodiments, the CTLA-4 inhibitor is ipilimumab or a biosimilar thereof, and the ipilimumab is administered at a dose of about 0.5 mg/kg to about 10 mg/kg. In some embodiments, the CTLA-4 inhibitor is ipilimumab or a biosimilar thereof, and the ipilimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the CTLA-4 inhibitor is ipilimumab or a biosimilar thereof, and the ipilimumab is administered at a dose of about 200 mg to about 500 mg. In some embodiments, the CTLA-4 inhibitor is ipilimumab or a biosimilar thereof, and the ipilimumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the CTLA-4 inhibitor is ipilimumab or a biosimilar thereof, and the ipilimumab is administered every 2 weeks, every 3 weeks, every 4 weeks, every 5 weeks, or every 6 weeks. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat unresectable or metastatic melanoma. In some embodiments, the ipilimumab is administered to treat Unresectable or Metastatic Melanoma at about mg/kg every 3 weeks for a maximum of 4 doses. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered for the adjuvant treatment of melanoma. In some embodiments, the ipilimumab is administered to for the adjuvant treatment of melanoma at about 10 mg/kg every 3 weeks for 4 doses, followed by 10 mg/kg every 12 weeks for up to 3 years. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat advanced renal cell carcinoma. In some embodiments, the ipilimumab is administered to treat advanced renal cell carcinoma at about 1 mg/kg immediately following nivolumab 3 mg/kg on the same day, every 3 weeks for 4 doses. In some embodiments, after completing 4 doses of the combination, nivolumab can be administered as a single agent according to standard dosing regimens for advanced renal cell carcinoma and/or renal cell carcinoma. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat microsatellite instability-high (MSI-H) or mismatch repair deficient (dMMR) metastatic colorectal cancer. In some embodiments, the ipilimumab is administered to treat microsatellite instability-high (MSI-H) or mismatch repair deficient (dMMR) metastatic colorectal cancer at about 1 mg/kg intravenously over 30 minutes immediately following nivolumab 3 mg/kg intravenously over 30 minutes on the same day, every 3 weeks for 4 doses. In some embodiments, after completing 4 doses of the combination, administer nivolumab as a single agent as recommended according to standard dosing regimens for microsatellite instability-high (MSI-H) or mismatch repair deficient (dMMR) metastatic colorectal cancer. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat hepatocellular carcinoma. In some embodiments, the ipilimumab is administered to treat hepatocellular carcinoma at about 3 mg/kg intravenously over 30 minutes immediately following nivolumab 1 mg/kg intravenously over 30 minutes on the same day, every 3 weeks for 4 doses. In some embodiments, after completion 4 doses of the combination, administer nivolumab as a single agent according to standard dosing regimens for hepatocellular carcinoma. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat metastatic non-small cell lung cancer. In some embodiments, the ipilimumab is administered to treat metastatic non-small cell lung cancer at about 1 mg/kg every 6 weeks with nivolumab 3 mg/kg every 2 weeks. In some embodiments, the ipilimumab is administered to treat metastatic non-small cell lung cancer at about 1 mg/kg every 6 weeks with nivolumab 360 mg every 3 weeks and 2 cycles of platinum-doublet chemotherapy. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat malignant pleural mesothelioma. In some embodiments, the ipilimumab is administered to treat malignant pleural mesothelioma at about 1 mg/kg every 6 weeks with nivolumab 360 mg every 3 weeks. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

Tremelimumab (also known as CP-675,206) is a fully human IgG2 monoclonal antibody and has the CAS number 745013-59-6. Tremelimumab is disclosed as antibody 11.2.1 in U.S. Pat. No. 6,682,736 (incorporated herein by reference). The amino acid sequences of the heavy chain and light chain of tremelimumab are set forth in SEQ ID NOs:218 and 219, respectively. Tremelimumab has been investigated in clinical trials for the treatment of various tumors, including melanoma and breast cancer; in which Tremelimumab was administered intravenously either as single dose or multiple doses every 4 or 12 weeks at the dose range of 0.01 and 15 mg/kg. In the regimens provided by the present invention, tremelimumab is administered locally, particularly intradermally or subcutaneously. The effective amount of tremelimumab administered intradermally or subcutaneously is typically in the range of 5-200 mg/dose per person. In some embodiments, the effective amount of tremelimumab is in the range of 10-150 mg/dose per person per dose. In some particular embodiments, the effective amount of tremelimumab is about 10, 25, 37.5, 40, 50, 75, 100, 125, 150, 175, or 200 mg/dose per person.

In an embodiment, a CTLA-4 inhibitor comprises a heavy chain given by SEQ ID NO:218 and a light chain given by SEQ ID NO:219. In an embodiment, a CTLA-4 inhibitor comprises heavy and light chains having the sequences shown in SEQ ID NO:218 and SEQ ID NO:219, respectively, or antigen binding fragments, Fab fragments, single-chain variable fragments (scFv), variants, or conjugates thereof. In an embodiment, a CTLA-4 inhibitor comprises heavy and light chains that are each at least 99% identical to the sequences shown in SEQ ID NO:218 and SEQ ID NO:219, respectively. In an embodiment, a CTLA-4 inhibitor comprises heavy and light chains that are each at least 98% identical to the sequences shown in SEQ ID NO:218 and SEQ ID NO:219, respectively. In an embodiment, a CTLA-4 inhibitor comprises heavy and light chains that are each at least 97% identical to the sequences shown in SEQ ID NO:218 and SEQ ID NO:219, respectively. In an embodiment, a CTLA-4 inhibitor comprises heavy and light chains that are each at least 96% identical to the sequences shown in SEQ ID NO:218 and SEQ ID NO:219, respectively. In an embodiment, a CTLA-4 inhibitor comprises heavy and light chains that are each at least 95% identical to the sequences shown in SEQ ID NO:218 and SEQ ID NO:219, respectively.

In an embodiment, the CTLA-4 inhibitor comprises the heavy and light chain CDRs or variable regions (VRs) of tremelimumab. In an embodiment, the CTLA-4 inhibitor heavy chain variable region (V_{H}) comprises the sequence shown in SEQ ID NO:220, and the CTLA-4 inhibitor light chain variable region (V_{L}) comprises the sequence shown in SEQ ID NO:221, or conservative amino acid substitutions thereof. In an embodiment, a CTLA-4 inhibitor comprises V_{H} and V_{L} regions that are each at least 99% identical to the sequences shown in SEQ ID NO:220 and SEQ ID NO:221, respectively. In an embodiment, a CTLA-4 inhibitor comprises V_{H} and V_{L} regions that are each at least 98% identical to the sequences shown in SEQ ID NO:220 and SEQ ID NO:221, respectively. In an embodiment, a CTLA-4 inhibitor comprises V_{H} and V_{L} regions that are each at least 97% identical to the sequences shown in SEQ ID NO:220 and SEQ ID NO:221, respectively. In an embodiment, a CTLA-4 inhibitor comprises V_{H} and V_{L} regions that are each at least 96% identical to the sequences shown in SEQ ID NO:220 and SEQ ID NO:221, respectively. In an embodiment, a CTLA-4 inhibitor comprises V_{H} and V_{L} regions that are each at least 95% identical to the sequences shown in SEQ ID NO:220 and SEQ ID NO:221, respectively.

In an embodiment, a CTLA-4 inhibitor comprises the heavy chain CDR1, CDR2 and CDR3 domains having the sequences set forth in SEQ ID NO:222, SEQ ID NO:223, and SEQ ID NO:224, respectively, or conservative amino acid substitutions thereof, and light chain CDR1, CDR2 and CDR3 domains having the sequences set forth in SEQ ID NO:225, SEQ ID NO:226, and SEQ ID NO:227, respectively, or conservative amino acid substitutions thereof. In an embodiment, the antibody competes for binding with, and/or binds to the same epitope on CTLA-4 as any of the aforementioned antibodies.

In an embodiment, the CTLA-4 inhibitor is an anti-CTLA-4 biosimilar monoclonal antibody approved by drug regulatory authorities with reference to tremelimumab. In an embodiment, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tremelimumab. In some embodiments, the one or more post-translational modifications are selected from one or more of glycosylation, oxidation, deamidation, and truncation. The amino acid sequences of tremelimumab are set forth in Table 24. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is tremelimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tremelimumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tremelimumab.

TABLE 24
Amino acid sequences for tremelimumab.

| Identifier | Sequence (One-Letter Amino Acid Symbols) | |
|---|---|---|
| SEQ ID NO: 218 | QVQLVESGGG VVQPGRSLRL SCAASGFTFS SYGMHWVRQA PGKGLEWVAV IWYDGSNKYY | 60 |
| tremelimumab | ADSVKGRFTI SRDNSKNTLY LQMNSLRAED TAVYYCARDP RGATLYYYYY GMDVWGQGTT | 120 |
| heavy chain | VTVSSASTKG PSVFPLAPCS RSTSESTAAL GCLVKDYFPE PVTVSWNSGA LTSGVHTFPA | 180 |
| | VLQSSGLYSL SSVVTVPSSN FGTQTYTCNV DHKPSNTKVD KTVERKCCVE CPPCPAPPVA | 240 |
| | GPSVFLFPPK PKDTLMISRT PEVTCVVVDV SHEDPEVQFN WYVDGVEVHN AKTKPREEQF | 300 |
| | NSTFRVVSVL TVVHQDWING KEYKCKVSNK GLPAPIEKTI SKTKGQPREP QVYTLPPSRE | 360 |
| | EMTKNQVSLT CLVKGFYPSD IAVEWESNGQ PENNYKTTPP MLDSDGSFFL YSKLTVDKSR | 420 |
| | WQQGNVFSCS VMHEALHNHY TQKSLSLSPG K | 451 |
| SEQ ID NO: 219 | DIQMTQSPSS LSASVGDRVT ITCRASQSIN SYLDWYQQKP GKAPKLLIYA ASSLQSGVPS | 60 |
| tremelimumab | RFSGSGSGTD FTLTISSLQP EDFATYYCQQ YYSTPFTFGP GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSEN RGEC | 214 |
| SEQ ID NO: 220 | GVVQPGRSLR LSCAASGFTF SSYGMEWVRQ APGKGLEWVA VIWYDGSNKY YADSVKGRFT | 60 |
| tremelimumab | ISRDNSKNTL YLQMNSLRAE DTAVYYCARD PRGASLYYYY YGMDVWGQGT TVTVSSASTK | 120 |
| variable heavy | GPSVFPLAPC SRSTSESTAA LGCLVKDYFP EPVTVSWNSG ALTSGVH | 167 |
| chain | | |
| SEQ ID NO: 221 | PSSLSASVGD RVTITCRASQ SINSYLDWYQ QKPGKAPKLL IYAASSLQSG VPSRFSGSGS | 60 |
| tremelimumab | GTDFTLTISS LQPEDFATYY CQQYYSTPFT FGPGTKVEIK RTVAAPSVFI FPPSDEQLKS | 120 |
| variable light | GTASVVCLLN NFYPREAKV | 139 |
| chain | | |
| SEQ ID NO: 222 | GFTFSSYGMH | 10 |
| tremelimumab | | |
| heavy chain | | |
| CDR1 | | |
| SEQ ID NO: 223 | VIWYDGSNKY YADSV | 15 |
| tremelimumab | | |
| heavy chain | | |
| CDR2 | | |
| SEQ ID NO: 224 | DPRGATLYYY YYGMDV | 16 |
| tremelimumab | | |
| heavy chain | | |
| CDR3 | | |
| SEQ ID NO: 225 | RASQSINSYL D | 11 |
| tremelimumab | | |
| light chain | | |
| CDR1 | | |
| SEQ ID NO: 226 | AASSLQS | 7 |
| tremelimumab | | |
| light chain | | |
| CDR2 | | |
| SEQ ID NO: 227 | QQYYSTPFT | 9 |
| tremelimumab | | |
| light chain | | |
| CDR3 | | |

In some embodiments, the CTLA-4 inhibitor is tremelimumab or a biosimilar thereof, and the tremelimumab is administered at a dose of about 0.5 mg/kg to about 10 mg/kg. In some embodiments, the CTLA-4 inhibitor is tremelimumab or a biosimilar thereof, and the tremelimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is tremelimumab or a biosimilar thereof, and the tremelimumab is administered at a dose of about 200 mg to about 500 mg. In some embodiments, the CTLA-4 inhibitor is tremelimumab or a biosimilar thereof, and the tremelimumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is tremelimumab or a biosimilar thereof, and the tremelimumab is administered every 2 weeks, every 3 weeks, every 4 weeks, every 5 weeks, or every 6 weeks. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In an embodiment, the CTLA-4 inhibitor is zalifrelimab from Agenus, or biosimilars, antigen-binding fragments, conjugates, or variants thereof. Zalifrelimab is a fully human monoclonal antibody. Zalifrelimab is assigned Chemical Abstracts Service (CAS) registry number 2148321-69-9 and is also known as also known as AGEN1884. The preparation and properties of zalifrelimab are described in U.S. Pat. No. 10,144,779 and US Patent Application Publication No. US2020/0024350 A1, the disclosures of which are incorporated by reference herein.

In an embodiment, a CTLA-4 inhibitor comprises a heavy chain given by SEQ ID NO:228 and a light chain given by SEQ ID NO:229. In an embodiment, a CTLA-4 inhibitor comprises heavy and light chains having the sequences shown in SEQ ID NO:228 and SEQ ID NO:229, respectively, or antigen binding fragments, Fab fragments, single-chain variable fragments (scFv), variants, or conjugates thereof. In an embodiment, a CTLA-4 inhibitor comprises heavy and light chains that are each at least 99% identical to the sequences shown in SEQ ID NO:228 and SEQ ID NO:229, respectively. In an embodiment, a CTLA-4 inhibitor comprises heavy and light chains that are each at least 98% identical to the sequences shown in SEQ ID NO:228 and SEQ ID NO:229, respectively. In an embodiment, a CTLA-4 inhibitor comprises heavy and light chains that are each at least 97% identical to the sequences shown in SEQ ID NO:228 and SEQ ID NO:229, respectively. In an embodiment, a CTLA-4 inhibitor comprises heavy and light chains that are each at least 96% identical to the sequences shown in SEQ ID NO:228 and SEQ ID NO:229, respectively. In an embodiment, a CTLA-4 inhibitor comprises heavy and light chains that are each at least 95% identical to the sequences shown in SEQ ID NO:228 and SEQ ID NO:229, respectively.

In an embodiment, the CTLA-4 inhibitor comprises the heavy and light chain CDRs or variable regions (VRs) of zalifrelimab. In an embodiment, the CTLA-4 inhibitor heavy chain variable region (V_{H}) comprises the sequence shown in SEQ ID NO:230, and the CTLA-4 inhibitor light chain variable region (V_{L}) comprises the sequence shown in SEQ ID NO:231, or conservative amino acid substitutions thereof. In an embodiment, a CTLA-4 inhibitor comprises V_{H} and V_{L} regions that are each at least 99% identical to the sequences shown in SEQ ID NO:230 and SEQ ID NO:231, respectively. In an embodiment, a CTLA-4 inhibitor comprises V_{H} and V_{L} regions that are each at least 98% identical to the sequences shown in SEQ ID NO:230 and SEQ ID NO:231, respectively. In an embodiment, a CTLA-4 inhibitor comprises V_{H} and V_{L} regions that are each at least 97% identical to the sequences shown in SEQ ID NO:230 and SEQ ID NO:231, respectively. In an embodiment, a CTLA-4 inhibitor comprises V_{H} and V_{L} regions that are each at least 96% identical to the sequences shown in SEQ ID NO:230 and SEQ ID NO:231, respectively. In an embodiment, a CTLA-4 inhibitor comprises V_{H} and V_{L} regions that are each at least 95% identical to the sequences shown in SEQ ID NO:230 and SEQ ID NO:231, respectively.

In an embodiment, a CTLA-4 inhibitor comprises the heavy chain CDR1, CDR2 and CDR3 domains having the sequences set forth in SEQ ID NO:231, SEQ ID NO:233, and SEQ ID NO:234, respectively, or conservative amino acid substitutions thereof, and light chain CDR1, CDR2 and CDR3 domains having the sequences set forth in SEQ ID NO:235, SEQ ID NO:236, and SEQ ID NO:237, respectively, or conservative amino acid substitutions thereof. In an embodiment, the antibody competes for binding with, and/or binds to the same epitope on CTLA-4 as any of the aforementioned antibodies.

In an embodiment, the CTLA-4 inhibitor is a CTLA-4 biosimilar monoclonal antibody approved by drug regulatory authorities with reference to zalifrelimab. In an embodiment, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is zalifrelimab. In some embodiments, the one or more post-translational modifications are selected from one or more of: glycosylation, oxidation, deamidation, and truncation. The amino acid sequences of zalifrelimab are set forth in Table 25. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is zalifrelimab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is zalifrelimab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is zalifrelimab.

TABLE 25
Amino acid sequences for zalifrelimab.

| Identifier | Sequence (One-Letter Amino Acid Symbols) | |
|---|---|---|
| SEQ ID NO: 228 | EVQLVESGGG LVKPGGSIRL SCAASGFTFS SYSMNWVRQA PGKGLEWVSS ISSSSSYIYY | 60 |
| zalifrelimab | ADSVKGRFTI SRDNAKNSLY LQMNSLRAED TAVYYCARVG LMGPFDIWGQ GTMVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKRVEPKS CDKTHTCPPC PAPELLGGPS | 240 |
| | VFLFPPKPKD TLMISRTPEV TCVVVDVSHE DPEVKENWYV DGVEVHNAKT KPREEQYNST | 300 |
| | YRVVSVLTVL HQDWLNGKEY KCKVSNKALP APIEKTISKA KGQPREPQVY TLPPSREEMT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPGK | 448 |
| SEQ ID NO: 229 | EIVLTQSPGT LSLSPGERAT LSCRASQSVS RYLGWYQQKP GQAPRLLIYG ASTRATGIPD | 60 |
| zalifrelimab | RFSGSGSGTD FTLTITRLEP EDFAVYYCQQ YGSSPWTFGQ GTKVEIKRTV AAPSVFIFPP | 120 |
| light chain | SDEQLKSGTA SVVCLINNFY PREAKVQWKV DNALQSGNSQ ESVTEQDSKD STYSLSSTLT | 180 |
| | LSKADYEKHK VYACEVTHQG LSSPVTKSEN RGEC | 214 |
| SEQ ID NO: 230 | EVQLVESGGG LVKPGGSIRL SCAASGFTFS SYSMNWVRQA PGKGLEWVSS ISSSSSYIYY | 60 |
| zalifrelimab | ADSVKGRFTI SRDNAKNSLY LQMNSLRAED TAVYYCARVG LMGPFDIWGQ GTMVTVSS | 118 |
| variable heavy | | |
| chain | | |
| SEQ ID NO: 231 | EIVLTQSPGT LSLSPGERAT LSCRASQSVS RYLGWYQQKP GQAPRLLIYG ASTRATGIPD | 60 |
| zalifrelimab | RFSGSGSGTD FTLTITRLEP EDFAVYYCQQ YGSSPWTFGQ GTKVEIK | 107 |
| variable light | | |
| chain | | |
| SEQ ID NO: 232 | GFTFSSYS | 8 |
| zalifrelimab | | |
| heavy chain | | |
| CDR1 | | |
| SEQ ID NO: 233 | ISSSSSYI | 8 |
| zalifrelimab | | |
| heavy chain | | |
| CDR2 | | |
| SEQ ID NO: 234 | ARVGLMGPFD I | 11 |
| zalifrelimab | | |
| heavy chain | | |
| CDR3 | | |
| SEQ ID NO: 235 | QSVSRY | 6 |
| zalifrelimab | | |
| light chain | | |
| CDR1 | | |
| SEQ ID NO: 236 | GAS | 3 |
| zalifrelimab | | |
| light chain | | |
| CDR2 | | |
| SEQ ID NO: 237 | QQYGSSPWT | 9 |
| zalifrelimab | | |
| light chain | | |
| CDR3 | | |

Examples of additional anti-CTLA-4 antibodies includes, but are not limited to: AGEN1181, BMS-986218, BCD-145, ONC-392, CS1002, REGN4659, and ADG116, which are known to one of ordinary skill in the art.

In some embodiments, the anti-CTLA-4 antibody is an anti-CTLA-4 antibody disclosed in any of the following patent publications: US 2019/0048096 A1; US 2020/0223907; US 2019/0201334; US 2019/0201334; US 2005/0201994; EP 1212422 B1; WO 2018/204760; WO 2018/204760; WO 2001/014424; WO 2004/035607; WO 2003/086459; WO 2012/120125; WO 2000/037504; WO 2009/100140; WO 2006/09649; WO2005092380; WO 2007/123737; WO 2006/029219; WO 2010/0979597; WO 2006/12168; and WO1997020574, each of which is incorporated herein by reference. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014; and/or U.S. Pat. Nos. 5,977,318, 6,682,736, 7,109,003, and 7,132,281, each of which is incorporated herein by reference. In some embodiments, the anti-CTLA-4 antibody is, for example, those disclosed in: WO 98/42752; U.S. Pat. Nos. 6,682,736 and 6,207,156; Hurwitz, et al., Proc. Natl. Acad. Sci. USA, 1998, 95, 10067-10071 (1998); Camacho, et al., J. Clin. Oncol., 2004, 22, 145 (Abstract No. 2505 (2004) (antibody CP-675206); or Mokyr, et al., Cancer Res., 1998, 58, 5301-5304 (1998), each of which is incorporated herein by reference.

In some embodiments, the CTLA-4 inhibitor is a CTLA-4 ligand as disclosed in WO 1996/040915 (incorporated herein by reference).

In some embodiments, the CTLA-4 inhibitor is a nucleic acid inhibitor of CTLA-4 expression. For example, anti-CTLA-4 RNAi molecules may take the form of the molecules described in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438 (incorporated herein by reference). In some instances, the anti-CTLA-4 RNAi molecules take the form of double stranded RNAi molecules described in European Patent No. EP 1309726 (incorporated herein by reference). In some instances, the anti-CTLA-4 RNAi molecules take the form of double stranded RNAi molecules described in U.S. Pat. Nos. 7,056,704 and 7,078,196 (incorporated herein by reference). In some embodiments, the CTLA-4 inhibitor is an aptamer described in International Patent Application Publication No. WO 2004/081021 (incorporated herein by reference).

In other embodiments, the anti-CTLA-4 RNAi molecules of the present invention are RNA molecules described in U.S. Pat. Nos. 5,898,031, 6,107,094, 7,432,249, and 7,432,250, and European Application No. EP 0928290 (incorporated herein by reference).

In some embodiments, the present invention includes a method of treating a patient with a cancer comprising the steps of administering a TIL regimen, wherein the TIL regimen includes a TIL product genetically modified to express a CCR, and further comprising the step of administering a CTLA-4 inhibitor. In some embodiments, the present invention includes a composition comprising (i) a TIL product genetically modified to express a CCR and (ii) a CTLA-4 inhibitor. In some embodiments, the present invention includes a kit comprising (i) a TIL product genetically modified to express a CCR and (ii) a CTLA-4 inhibitor.

In some embodiments, the present invention includes a method of treating a patient with a cancer comprising the steps of administering a TIL regimen, wherein the TIL regimen includes a TIL product genetically modified to express a CCR, and further comprising the steps of administering a CTLA-4 inhibitor and either a PD-1 inhibitor or a PD-L1 inhibitor. In some embodiments, the present invention includes a composition comprising (i) a TIL product genetically modified to express a CCR, (ii) a CTLA-4 inhibitor, and (iii) either a PD-1 inhibitor or a PD-L1 inhibitor. In some embodiments, the present invention includes a kit comprising (i) a TIL product genetically modified to express a CCR, (ii) a CTLA-4 inhibitor, and (iii) either a PD-1 inhibitor or a PD-L1 inhibitor.

### 4. Lymphodepletion Preconditioning of Patients

In an embodiment, the invention includes a method of treating a cancer with a population of TILs, wherein a patient is pre-treated with non-myeloablative chemotherapy prior to an infusion of TILs according to the present disclosure. In an embodiment, the invention includes a population of TILs for use in the treatment of cancer in a patient which has been pre-treated with non-myeloablative chemotherapy. In an embodiment, the population of TILs is for administration by infusion. In an embodiment, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In an embodiment, after non-myeloablative chemotherapy and TIL infusion (at day 0) according to the present disclosure, the patient receives an intravenous infusion of IL-2 (aldesleukin, commercially available as PROLEUKIN) intravenously at 720,000 IU/kg every 8 hours to physiologic tolerance. In certain embodiments, the population of TILs is for use in treating cancer in combination with IL-2, wherein the IL-2 is administered after the population of TILs.

Experimental findings indicate that lymphodepletion prior to adoptive transfer of tumor-specific T lymphocytes plays a key role in enhancing treatment efficacy by eliminating regulatory T cells and competing elements of the immune system (‘cytokine sinks’). Accordingly, some embodiments of the invention utilize a lymphodepletion step (sometimes also referred to as “immunosuppressive conditioning”) on the patient prior to the introduction of the TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J. Clin. Oncol. 2008, 26, 5233-5239, and Dudley, et al., J. Clin. Oncol. 2005, 23, 2346-2357, all of which are incorporated by reference herein in their entireties.

In some embodiments, the fludarabine is administered at a concentration of 0.5 μg/mL to 10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 g/mL-10 g/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide together to a patient. In some embodiments, fludarabine is administered at 25 mg/m²/day i.v. and cyclophosphamide is administered at 250 mg/m²/day i.v. over 4 days.

In an embodiment, the lymphodepletion is performed by administration of cyclophosphamide at a dose of 60 mg/m²/day for two days followed by administration of fludarabine at a dose of 25 mg/m²/day for five days.

In an embodiment, the lymphodepletion is performed by administration of cyclophosphamide at a dose of 60 mg/m²/day for two days and administration of fludarabine at a dose of 25 mg/m²/day for five days, wherein cyclophosphamide and fludarabine are both administered on the first two days, and wherein the lymphodepletion is performed in five days in total.

In an embodiment, the lymphodepletion is performed by administration of cyclophosphamide at a dose of about 50 mg/m²/day for two days and administration of fludarabine at a dose of about 25 mg/m²/day for five days, wherein cyclophosphamide and fludarabine are both administered on the first two days, and wherein the lymphodepletion is performed in five days in total.

In an embodiment, the lymphodepletion is performed by administration of cyclophosphamide at a dose of about 50 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In an embodiment, the lymphodepletion is performed by administration of cyclophosphamide at a dose of about 40 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In an embodiment, the lymphodepletion is performed by administration of cyclophosphamide at a dose of about 40 mg/m²/day for two days and administration of fludarabine at a dose of about 15 mg/m²/day for five days, wherein cyclophosphamide and fludarabine are both administered on the first two days, and wherein the lymphodepletion is performed in five days in total.

In an embodiment, the lymphodepletion is performed by administration of cyclophosphamide at a dose of 60 mg/m²/day and fludarabine at a dose of 25 mg/m²/day for two days followed by administration of fludarabine at a dose of 25 mg/m²/day for three days.

In an embodiment, the cyclophosphamide is administered with mesna. In an embodiment, mesna is administered at 15 mg/kg. In an embodiment where mesna is infused, and if infused continuously, mesna can be infused over approximately 2 hours with cyclophosphamide (on Days −5 and/or −4), then at a rate of 3 mg/kg/hour for the remaining 22 hours over the 24 hours starting concomitantly with each cyclophosphamide dose.

In an embodiment, the lymphodepletion further comprises the step of treating the patient with an IL-2 regimen starting on the day after administration of the third population of TILs to the patient.

In an embodiment, the lymphodepletion further comprises the step of treating the patient with an IL-2 regimen starting on the same day as administration of the third population of TILs to the patient.

In some embodiments, the lymphodeplete comprises 5 days of preconditioning treatment. In some embodiments, the days are indicated as days −5 through −1, or Day 0 through Day 4. In some embodiments, the regimen comprises cyclophosphamide on days −5 and −4 (i.e., days 0 and 1). In some embodiments, the regimen comprises intravenous cyclophosphamide on days −5 and −4 (i.e., days 0 and 1). In some embodiments, the regimen comprises 60 mg/kg intravenous cyclophosphamide on days −5 and −4 (i.e., days 0 and 1). In some embodiments, the cyclophosphamide is administered with mesna. In some embodiments, the regimen further comprises fludarabine. In some embodiments, the regimen further comprises intravenous fludarabine. In some embodiments, the regimen further comprises 25 mg/m² intravenous fludarabine. In some embodiments, the regimen further comprises 25 mg/m² intravenous fludarabine on days −5 and −1 (i.e., days 0 through 4). In some embodiments, the regimen further comprises 25 mg/m² intravenous fludarabine on days −5 and −1 (i.e., days 0 through 4).

In some embodiments, the non-myeloablative lymphodepletion regimen comprises the steps of administration of cyclophosphamide at a dose of 60 mg/m2/day and fludarabine at a dose of 25 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m2/day and fludarabine at a dose of 25 mg/m²/day for two days followed by administration of fludarabine at a dose of 25 mg/m²/day for three days.

In some embodiments, the non-myeloablative lymphodepletion regimen is administered according to Table 26.

TABLE 26
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 60 mg/kg | | | | | | |
| Mesna (as needed) | X | X | | | | |
| Fludarabine | X | X | X | X | X | |
| 25 mg/m²/day | | | | | | |
| TIL infusion | | | | | | X |

In some embodiments, the non-myeloablative lymphodepletion regimen is administered according to Table 27.

TABLE 27
Exemplary lymphodepletion and treatment regimen.

| | | | | | | | | | |
|---|---|---|---|---|---|---|---|---|---|
| Day | −4 | −3 | −2 | −1 | 0 | 1 | 2 | 3 | 4 |

| | | | | | |
|---|---|---|---|---|---|
| Cyclophosphamide 60 mg/kg | X | X | | | |
| Mesna (as needed) | X | X | | | |
| Fludarabine 25 mg/m²/day | X | X | X | X | |
| TIL infusion | | | | | X |

In some embodiments, the non-myeloablative lymphodepletion regimen is administered according to Table 28.

TABLE 28
Exemplary lymphodepletion and treatment regimen.

| | | | | | | | | |
|---|---|---|---|---|---|---|---|---|
| Day | −3 | −2 | −1 | 0 | 1 | 2 | 3 | 4 |

| | | | | |
|---|---|---|---|---|
| Cyclophosphamide 60 mg/kg | X | X | | |
| Mesna (as needed) | X | X | | |
| Fludarabine 25 mg/m²/day | X | X | X | |
| TIL infusion | | | | X |

In some embodiments, the non-myeloablative lymphodepletion regimen is administered according to Table 29.

TABLE 29
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 60 mg/kg | | | | | | |
| Mesna (as needed) | X | X | | | | |
| Fludarabine | | | X | X | X | |
| 25 mg/m²/day | | | | | | |
| TIL infusion | | | | | | X |

In some embodiments, the non-myeloablative lymphodepletion regimen is administered according to Table 30.

TABLE 30
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 300 mg/kg | | | | | | |
| Mesna (as needed) | X | X | | | | |
| Fludarabine | X | X | X | X | X | |
| 30 mg/m²/day | | | | | | |
| TIL infusion | | | | | | X |

In some embodiments, the non-myeloablative lymphodepletion regimen is administered according to Table 31.

TABLE 31
Exemplary lymphodepletion and treatment regimen.

| | | | | | | | | | |
|---|---|---|---|---|---|---|---|---|---|
| Day | −4 | −3 | −2 | −1 | 0 | 1 | 2 | 3 | 4 |

| | | | | | |
|---|---|---|---|---|---|
| Cyclophosphamide 300 mg/kg | X | X | | | |
| Mesna (as needed) | X | X | | | |
| Fludarabine 30 mg/m²/day | X | X | X | X | |
| TIL infusion | | | | | X |

In some embodiments, the non-myeloablative lymphodepletion regimen is administered according to Table 32.

TABLE 32
Exemplary lymphodepletion and treatment regimen.

| | | | | | | | | |
|---|---|---|---|---|---|---|---|---|
| Day | −3 | −2 | −1 | 0 | 1 | 2 | 3 | 4 |

| | | | | |
|---|---|---|---|---|
| Cyclophosphamide 300 mg/kg | X | X | | |
| Mesna (as needed) | X | X | | |
| Fludarabine 30 mg/m²/day | X | X | X | |
| TIL infusion | | | | X |

In some embodiments, the non-myeloablative lymphodepletion regimen is administered according to Table 33.

TABLE 33
Exemplary lymphodepletion and treatment regimen.

| | | | | | | | | | | |
|---|---|---|---|---|---|---|---|---|---|---|
| Day | −5 | −4 | −3 | −2 | −1 | 0 | 1 | 2 | 3 | 4 |

| | | | | | | |
|---|---|---|---|---|---|---|
| Cyclophosphamide | X | X | | | | |
| 300 mg/kg | | | | | | |
| Mesna (as needed) | X | X | | | | |
| Fludarabine | | | X | X | X | |
| 30 mg/m²/day | | | | | | |
| TIL infusion | | | | | | X |

In some embodiments, the TIL infusion used with the foregoing embodiments of myeloablative lymphodepletion regimens may be any TIL composition described herein, including TIL products genetically modified to express a CCR as described herein, and may also include infusions of MILs and PBLs in place of the TIL infusion, as well as the addition of IL-2 regimens and administration of co-therapies (such as PD-1 and PD-L1 inhibitors) as described herein.

In some embodiments, the non-myeloablative lymphodepletion regimen comprises melphalan administered to a total dose of 100 mg/m² over the course of 1, 2, or 3 days prior to the day of TIL infusion. In some embodiments, the non-myeloablative lymphodepletion regimen comprises melphalan administered to a total dose of 200 mg/m² over the course of 1, 2, or 3 days prior to the day of TIL infusion. In some embodiments, the non-myeloablative lymphodepletion regimen comprises melphalan administered to a total dose of 100 mg/m² and fludarabine administered at a dose of 30 mg/m²/day over the course of 1, 2, or 3 days prior to the day of TIL infusion. In some embodiments, the non-myeloablative lymphodepletion regimen comprises melphalan administered to a total dose of 200 mg/m² and fludarabine administered at a dose of 30 mg/m²/day over the course of 1, 2, or 3 days prior to the day of TIL infusion.

In some embodiments, the non-myeloablative lymphodepletion regimen comprises administration of an anti-CD45 antibody. In some embodiments, the non-myeloablative lymphodepletion regimen comprises administration of an anti-CD45 antibody-drug conjugate. In some embodiments, the non-myeloablative lymphodepletion regimen comprises administration of an anti-CD45 antibody-radioisotope conjugate. In some embodiments, the non-myeloablative lymphodepletion regimen comprises administration of apamistamab-¹³¹I. In some embodiments, the non-myeloablative lymphodepletion regimen comprises apamistamab-¹³¹I administered at a dose of 25 mCi, 50 mCi, 75 mCi, 100 mCi, 150 mCi, or 200 mCi between 2 and 9 days prior to TIL infusion. In some embodiments, the non-myeloablative lymphodepletion regimen comprises apamistamab-¹³¹I administered at a dose of 25 mCi to 200 mCi between 2 and 9 days prior to TIL infusion. In some embodiments, the non-myeloablative lymphodepletion regimen comprises apamistamab-¹³¹I administered at a dose of 50 mCi to 150 mCi between 4 and 8 days prior to TIL infusion. In some embodiments, the non-myeloablative lymphodepletion regimen comprises apamistamab-¹³¹I administered at a dose of about 75 mCi about 6 days prior to TIL infusion. In some embodiments, the non-myeloablative lymphodepletion regimen comprises apamistamab-¹³¹I administered at a dose of about 100 mCi about 7 days prior to TIL infusion.

In some embodiments, the TIL infusion used with the foregoing embodiments of myeloablative lymphodepletion regimens may be any TIL composition described herein, including TIL products genetically modified to express a CCR as described herein, and may also include infusions of MILs and PBLs in place of the TIL infusion, as well as the addition of alternative lymphodepletion regimens including the anti-CD52 antibody alemtuzumab, or variants, fragments, antibody-drug conjugates, or biosimilars thereof.

### 5. IL-2 Regimens

In an embodiment, the IL-2 regimen comprises a high-dose IL-2 regimen, wherein the high-dose IL-2 regimen comprises aldesleukin, or a biosimilar or variant thereof, administered intravenously starting on the day after administering a therapeutically effective portion of therapeutic population of TILs, wherein the aldesleukin or a biosimilar or variant thereof is administered at a dose of 0.037 mg/kg or 0.044 mg/kg IU/kg (patient body mass) using 15-minute bolus intravenous infusions every eight hours until tolerance, for a maximum of 14 doses. Following 9 days of rest, this schedule may be repeated for another 14 doses, for a maximum of 28 doses in total. In some embodiments, IL-2 is administered in 1, 2, 3, 4, 5, or 6 doses. In some embodiments, IL-2 is administered at a maximum dosage of up to 6 doses.

In an embodiment, the IL-2 regimen comprises a decrescendo IL-2 regimen. Decrescendo IL-2 regimens have been described in O'Day, et al., J. Clin. Oncol. 1999, 17, 2752-61 and Eton, et al., Cancer 2000, 88, 1703-9, the disclosures of which are incorporated herein by reference. In an embodiment, a decrescendo IL-2 regimen comprises 18×10⁶ IU/m² aldesleukin, or a biosimilar or variant thereof, administered intravenously over 6 hours, followed by 18×10⁶ IU/m² administered intravenously over 12 hours, followed by 18×10⁶ IU/m² administered intravenously over 24 hours, followed by 4.5×10⁶ IU/m² administered intravenously over 72 hours. This treatment cycle may be repeated every 28 days for a maximum of four cycles. In an embodiment, a decrescendo IL-2 regimen comprises 18,000,000 IU/m² on day 1, 9,000,000 IU/m² on day 2, and 4,500,000 IU/m² on days 3 and 4.

In an embodiment, the IL-2 regimen comprises a low-dose IL-2 regimen. Any low-dose IL-2 regimen known in the art may be used, including the low-dose IL-2 regimens described in Dominguez-Villar and Hafler, Nat. Immunology 2000, 19, 665-673; Hartemann, et al., Lancet Diabetes Endocrinol. 2013, 1, 295-305; and Rosenzwaig, et al., Ann. Rheum. Dis. 2019, 78, 209-217, the disclosures of which are incorporated herein by reference. In an embodiment, a low-dose IL-2 regimen comprises 18×10⁶ IU per m² of aldesleukin, or a biosimilar or variant thereof, per 24 hours, administered as a continuous infusion for 5 days, followed by 2-6 days without IL-2 therapy, optionally followed by an additional 5 days of intravenous aldesleukin or a biosimilar or variant thereof, as a continuous infusion of 18×10⁶ IU per m² per 24 hours, optionally followed by 3 weeks without IL-2 therapy, after which additional cycles may be administered.

In an embodiment, the IL-2 regimen comprises administration of pegylated IL-2 every 1, 2, 4, 6, 7, 14 or 21 days at a dose of 0.10 mg/day to 50 mg/day. In an embodiment, the IL-2 regimen comprises administration of bempegaldesleukin, or a fragment, variant, or biosimilar thereof, every 1, 2, 4, 6, 7, 14 or 21 days at a dose of 0.10 mg/day to 50 mg/day.

In an embodiment, the IL-2 regimen comprises administration of THOR-707, or a fragment, variant, or biosimilar thereof, every 1, 2, 4, 6, 7, 14 or 21 days at a dose of 0.10 mg/day to 50 mg/day.

In an embodiment, the IL-2 regimen comprises administration of nemvaleukin alfa, or a fragment, variant, or biosimilar thereof, every 1, 2, 4, 6, 7, 14 or 21 days at a dose of 0.10 mg/day to 50 mg/day.

In an embodiment, the IL-2 regimen comprises administration of an IL-2 fragment engrafted onto an antibody backbone. In an embodiment, the IL-2 regimen comprises administration of an antibody-cytokine engrafted protein that binds the IL-2 low affinity receptor. In an embodiment, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the antibody cytokine engrafted protein preferentially expands T effector cells over regulatory T cells. In an embodiment, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the IL-2 molecule is a mutein, and wherein the antibody cytokine engrafted protein preferentially expands T effector cells over regulatory T cells. In an embodiment, the IL-2 regimen comprises administration of an antibody comprising a heavy chain selected from the group consisting of SEQ ID NO:29 and SEQ ID NO:38 and a light chain selected from the group consisting of SEQ ID NO:37 and SEQ ID NO:39, or a fragment, variant, or biosimilar thereof, every 1, 2, 4, 6, 7, 14 or 21 days at a dose of 0.10 mg/day to 50 mg/day.

In some embodiments, the antibody cytokine engrafted protein described herein has a longer serum half-life that a wild-type IL-2 molecule such as, but not limited to, aldesleukin (Proleukin®) or a comparable molecule.

In an embodiment, the IL-2 regimen comprises administration of an IL-2 fragment engrafted onto an antibody backbone. In an embodiment, the IL-2 regimen comprises administration of an antibody-cytokine engrafted protein that binds the IL-2 low affinity receptor. In an embodiment, the IL-2 regimen comprises administration of an antibody-cytokine engrafted protein that exhibits enhanced binding to the IL-2Rβ and/or IL-2Rγ receptors, in comparison to aldesleukin, without an effect on the binding to the IL-2Rα receptor.

In some embodiments, the TIL infusion used with the foregoing embodiments of myeloablative lymphodepletion regimens may be any TIL composition described herein and may also include infusions of MILs and PBLs in place of the TIL infusion, as well as the addition of IL-2 regimens and administration of co-therapies (such as PD-1 and PD-L1 inhibitors) as described herein.

In some embodiments, the present invention includes a method of treating a patient with a cancer comprising the step of administering a TIL regimen, wherein the TIL regimen includes a TIL product genetically modified to express a CCR, and further comprising the step of administering an IL-2 regimen. In some embodiments, the present invention includes a composition comprising (i) a TIL product genetically modified to express a CCR and (ii) an IL-2 regimen. In some embodiments, the present invention includes a kit comprising (i) a TIL product genetically modified to express a CCR and (ii) an IL-2 regimen.

In some embodiments, the present invention includes a method of treating a patient with a cancer comprising the steps of administering a TIL regimen, wherein the TIL regimen includes a TIL product genetically modified to express a CCR, and further comprising the steps of administering an IL-2 regimen and either a PD-1 inhibitor or a PD-L1 inhibitor. In some embodiments, the present invention includes a composition comprising (i) a TIL product genetically modified to express a CCR, (ii) an IL-2 regimen, and (iii) either a PD-1 inhibitor or a PD-L1 inhibitor. In some embodiments, the present invention includes a kit comprising (i) a TIL product genetically modified to express a CCR, (ii) an IL-2 regimen, and (iii) either a PD-1 inhibitor or a PD-L1 inhibitor.

In some embodiments, the present invention includes a method of treating a patient with a cancer comprising the steps of administering a TIL regimen, wherein the TIL regimen includes a TIL product genetically modified to express a CCR, and further comprising the steps of administering a CTLA-4 inhibitor and an IL-2 regimen. In some embodiments, the present invention includes a composition comprising (i) a TIL product genetically modified to express a CCR, (ii) a CTLA-4 inhibitor, and (iii) an IL-2 regimen. In some embodiments, the present invention includes a kit comprising (i) a TIL product genetically modified to express a CCR, (ii) a CTLA-4 inhibitor, and (iii) an IL-2 regimen.

In some embodiments, the present invention includes a method of treating a patient with a cancer comprising the steps of administering a TIL regimen, wherein the TIL regimen includes a TIL product genetically modified to express a CCR, and further comprising the steps of administering an IL-2 regimen, a CTLA-4 inhibitor, and either a PD-1 inhibitor or a PD-L1 inhibitor. In some embodiments, the present invention includes a composition comprising (i) a TIL product genetically modified to express a CCR, (ii) an IL-2 regimen, (iii) either a PD-1 inhibitor or a PD-L1 inhibitor, and (iv) a CTLA-4 inhibitor. In some embodiments, the present invention includes a kit comprising (i) a TIL product genetically modified to express a CCR, (ii) an IL-2 regimen, (iii) either a PD-1 inhibitor or a PD-L1 inhibitor, and (iv) a CTLA-4 inhibitor.

### VIII. Chimeric Costimulatory Receptors

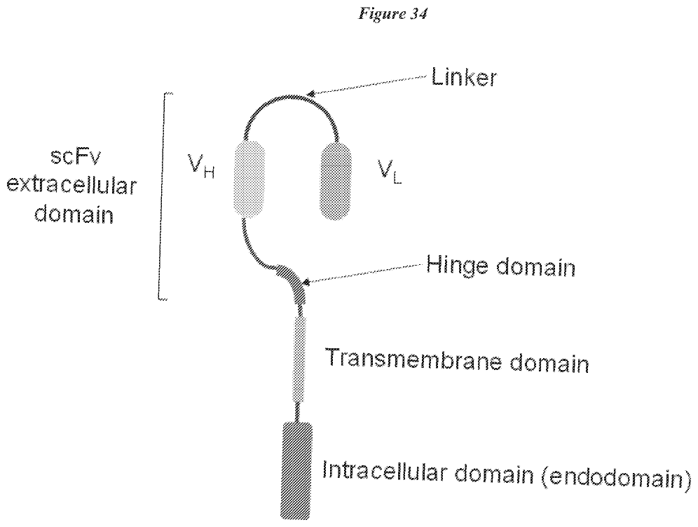

In some embodiments, the foregoing manufacturing processes, including Gen 2 and Gen 3 and other processes of making TILs, MILs, and PBLs, may be modified to include a step comprising the viral or non-viral transduction of TILs, MILs, or PBLs to express one or more CCRs described herein. In an embodiment, a CCR comprises an extracellular binding domain and an intracellular signaling domain. In an embodiment, a CCR comprises an extracellular binding domain and one or more intracellular signaling domains. In an embodiment, a CCR comprises an extracellular binding domain, a transmembrane domain, and an intracellular signaling domain. In an embodiment, a CCR comprises an extracellular binding domain, a hinge domain, a transmembrane domain, and an intracellular signaling domain. In an embodiment, the CCR is a single polypeptide containing multiple linked domains. In some embodiments, the CCR is a switch receptor. In some embodiments, the CCR includes one or more polypeptide domains as described in U.S. Patent Application Publication No. US 2019/0388468 A1, the disclosure of which is incorporated by reference herein. In other embodiments, the CCR includes one or more polypeptide domains as described in International Patent Application Publication No. WO 2020/152451 A1, the disclosure of which is incorporated by reference herein. In some embodiments, a CCR of the present invention, used in combination with the TIL, MIL, or PBL manufacturing processes described herein, is of the format shown in FIG. 34. In some embodiments, a CCR a CCR of the present invention, used in combination with the TIL, MIL, or PBL manufacturing processes described herein, includes the domains shown in FIG. 34, operatively linked to each other as shown in FIG. 34.

### A. Extracellular Domains

In an embodiment, a CCR comprises an extracellular domain. In an embodiment, a CCR comprises an extracellular domain that binds to a tumor-associated protein. In an embodiment, the extracellular domain binds to a tumor-associated cell surface molecule. In an embodiment, the extracellular domain binds to a tumor-associated extracellular molecule. In an embodiment, the extracellular domain binds to a tumor-associated antigen. In an embodiment, the extracellular domain binds to PD-L1, also known as CD274 and coded by PDCD1. In an embodiment, the extracellular domain is a PD-1 domain that binds to PD-L1, also known as CD274. In an embodiment, the extracellular domain binds to a tumor-associated antigen, wherein the tumor-associated antigen is a neoantigen. In an embodiment, the extracellular domain binds to a tumor-associated antigen, wherein the tumor-associated antigen is a peptide-major histocompatibility complex. In an embodiment, the extracellular domain binds to a tumor-associated antigen, wherein the tumor-associated antigen is a heat-shock protein peptide complex. In an embodiment, the extracellular domain binds to a protein selected from the group consisting of DLL3, CD19, CD20, CD22, CD24, CD33, CD38, CD39, CD73, CD123, CD138, CD228, LRRC15, CEA, FRα, EPCAM (CD326), PD-1, PD-L1 (CD274), PSMA, gp100, MUC1, MCSP, EGFR, GD2, TROP-2, GPC3, MICA, MICB, VISTA, ULBP, HER2, MCM5, FAP, 5T4, LFA-1, B7-H3, and MUC16.

In some embodiments, the extracellular binding includes a scFv capable of binding to a tumor-associated antigen. In some embodiments, the scFv includes V_{H} and V_{L} chains capable of binding to a protein selected from the group consisting of DLL3, CD19, CD20, CD22, CD24, CD33, CD38, CD39, CD73, CD123, CD138, CD228, LRRC15, CEA, FRα, EPCAM (CD326), PD-1, PD-L1 (CD274), PSMA, gp100, MUC1, MCSP, EGFR, GD2, TROP-2, GPC3, MICA, MICB, VISTA, ULBP, HER2, MCM5, FAP, 5T4, B7-H3, and MUC16. In some embodiments, the present invention includes modifications of a scFv amino acid sequence disclosed herein to generate functionally equivalent molecules, such as through conservative amino acid substitution. For example, the V_{H} or V_{L} of an scFv binding domain comprised within the CCR can be modified to retain at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity of the starting V_{H} or V_{L} framework region of scFv. The present invention also includes modifications of the entire CCR construct, such modifications in one or more amino acid sequences of the various domains of the CCR construct in order to generate functionally equivalent molecules.

In an embodiment, the extracellular domain binds to a human tumor-associated antigen. In an embodiment, the extracellular domain binds to a murine tumor-associated cell surface molecule.

In some embodiments, the extracellular domain binds a human or murine tumor-associated antigen with a K_{D} of about 100 pM or lower, binds a human or murine tumor-associated antigen with a K_{D} of about 90 pM or lower, binds a human or murine tumor-associated antigen with a K_{D} of about 80 pM or lower, binds a human or murine tumor-associated antigen with a K_{D} of about 70 pM or lower, binds a human or murine tumor-associated antigen with a K_{D} of about 60 pM or lower, binds a human or murine tumor-associated antigen with a K_{D} of about 50 pM or lower, binds a human or murine tumor-associated antigen with a K_{D} of about 40 pM or lower, binds a human or murine tumor-associated antigen with a K_{D} of about 30 pM or lower, binds a human or murine tumor-associated antigen with a K_{D} of about 20 pM or lower, or binds a human or murine tumor-associated antigen with a K_{D} of about 10 pM or lower.

In some embodiments, the extracellular domain binds a human or murine tumor-associated antigen with a k_{assoc} of about 7.5×10⁵ l/M·s or faster, binds a human or murine tumor-associated antigen with a k_{assoc} of about 7.5×10⁵ l/M·s or faster, binds a human or murine tumor-associated antigen with a k_{assoc} of about 8×10⁵ l/M·s or faster, binds a human or murine tumor-associated antigen with a k_{assoc} of about 8.5×10⁵ l/M·s or faster, binds a human or murine tumor-associated antigen with a k_{assoc} of about 9×10⁵ l/M·s or faster, binds a human or murine tumor-associated antigen with a k_{assoc} of about 9.5×10⁵ l/M·s or faster, or binds a human or murine tumor-associated antigen with a k_{assoc} of about 1×10⁶ l/M·s or faster.

In some embodiments, the extracellular domain binds a human or murine tumor-associated antigen with a k_{dissoc} of about 2×10⁻⁵ l/s or slower, binds a human or murine tumor-associated antigen with a k_{dissoc} of about 2.1×10⁻⁵ l/s or slower, binds a human or murine tumor-associated antigen with a k_{dissoc} of about 2.2×10⁻⁵ l/s or slower, binds a human or murine tumor-associated antigen with a k_{dissoc} of about 2.3×10⁻⁵ l/s or slower, binds a human or murine tumor-associated antigen with a k_{dissoc} of about 2.4×10⁻⁵ l/s or slower, binds a human or murine tumor-associated antigen with a k_{dissoc} of about 2.5×10⁻⁵ l/s or slower, binds a human or murine tumor-associated antigen with a k_{dissoc} of about 2.6×10⁻⁵ l/s or slower, binds a human or murine tumor-associated antigen with a k_{dissoc} of about 2.7×10⁻⁵ l/s or slower, binds a human or murine tumor-associated antigen with a k_{dissoc} of about 2.8×10⁻⁵ l/s or slower, binds a human or murine tumor-associated antigen with a k_{dissoc} of about 2.9×10⁻⁵ l/s or slower, or binds a human or murine tumor-associated antigen with a k_{dissoc} of about 3×10⁻⁵ l/s or slower.

Linker sequences suitable for use in conjunction with V_{H} and V_{L} domains described herein as an extracellular scFv domain for a CCR are given in Table 34. In an embodiment, a CCR of the present invention comprises an extracellular domain that comprises an scFv that comprises a V_{H} binding domain linked to a V_{L} binding domain by a linker sequence. In some embodiments, an scFv of the present invention is of the format: (V_{H})-(scFv linker)-(V_{L})-(remainder of construct). In some embodiments, an scFv of the present invention is of the format: (V_{L})-(linker)-(V_{H})-(remainder of construct). In some embodiments, a linker is chosen from the linkers given in Table 34. In some embodiments, the linker is SEQ ID NO:238, or conservative amino acid substitutions thereof, or a sequence with greater than 80%, greater than 85%, greater than 90%, or greater than 95% homology thereto. In some embodiments, the linker is SEQ ID NO:239, or conservative amino acid substitutions thereof, or a sequence with greater than 80%, greater than 85%, greater than 90%, or greater than 95% homology thereto. In some embodiments, the linker is SEQ ID NO:240, or conservative amino acid substitutions thereof, or a sequence with greater than 80%, greater than 85%, greater than 90%, or greater than 95% homology thereto. In some embodiments, the linker is SEQ ID NO:241, or conservative amino acid substitutions thereof, or a sequence with greater than 80%, greater than 85%, greater than 90%, or greater than 95% homology thereto. In some embodiments, the linker is SEQ ID NO:242, or conservative amino acid substitutions thereof, or a sequence with greater than 80%, greater than 85%, greater than 90%, or greater than 95% homology thereto. In some embodiments, the linker is SEQ ID NO:243, or conservative amino acid substitutions thereof, or a sequence with greater than 80%, greater than 85%, greater than 90%, or greater than 95% homology thereto.

TABLE 34
Amino acid sequences of scFv linker sequences.

| Identifier | Sequence (One-Letter Amino Acid Symbols) | |
|---|---|---|
| SEQ ID NO: 238 | GGGGS | 5 |
| scFv linker | | |
| SEQ ID NO: 239 | GGGGSGGGGS GGGGSGGGGS GGGGSGGGGS | 30 |
| scEv linker | | |
| SEQ ID NO: 240 | GGGGSGGGGS GGGGSGGGGS | 20 |
| scEv linker | | |
| SEQ ID NO: 241 | GGGGSGGGGS GGGGS | 15 |
| scFv linker | | |
| SEQ ID NO: 242 | GGGS | 4 |
| scFv linker | | |
| SEQ ID NO: 243 | GGGSGGGSGG GSGGGSGGGS GGGSGGGSGG GSGGGSGGGS | 40 |
| scFv linker | | |

Additional linker sequences suitable for use in conjunction with V_{H} and V_{L} domains described herein as an extracellular scFv domain for a CCR are given in Table 8. In some embodiments, the linker is SEQ ID NO:63, or conservative amino acid substitutions thereof, or a sequence with greater than 80%, greater than 85%, greater than 90%, or greater than 95% homology thereto. In some embodiments, the linker is SEQ ID NO:64, or conservative amino acid substitutions thereof, or a sequence with greater than 80%, greater than 85%, greater than 90%, or greater than 95% homology thereto. In some embodiments, the linker is SEQ ID NO:65, or conservative amino acid substitutions thereof, or a sequence with greater than 80%, greater than 85%, greater than 90%, or greater than 95% homology thereto. In some embodiments, the linker is SEQ ID NO:66, or conservative amino acid substitutions thereof, or a sequence with greater than 80%, greater than 85%, greater than 90%, or greater than 95% homology thereto. In some embodiments, the linker is SEQ ID NO:67, or conservative amino acid substitutions thereof, or a sequence with greater than 80%, greater than 85%, greater than 90%, or greater than 95% homology thereto. In some embodiments, the linker is SEQ ID NO:68, or conservative amino acid substitutions thereof, or a sequence with greater than 80%, greater than 85%, greater than 90%, or greater than 95% homology thereto. In some embodiments, the linker is SEQ ID NO:69, or conservative amino acid substitutions thereof, or a sequence with greater than 80%, greater than 85%, greater than 90%, or greater than 95% homology thereto. In some embodiments, the linker is SEQ ID NO:70, or conservative amino acid substitutions thereof, or a sequence with greater than 80%, greater than 85%, greater than 90%, or greater than 95% homology thereto. In some embodiments, the linker is SEQ ID NO:71, or conservative amino acid substitutions thereof, or a sequence with greater than 80%, greater than 85%, greater than 90%, or greater than 95% homology thereto. In some embodiments, the linker is SEQ ID NO:72, or conservative amino acid substitutions thereof, or a sequence with greater than 80%, greater than 85%, greater than 90%, or greater than 95% homology thereto.

Additional linker sequences suitable for use in conjunction with V_{H} and V_{L} domains in an scFv described herein as an extracellular domain for a CCR are given in Table 9. In some embodiments, the linker is SEQ ID NO:74, or conservative amino acid substitutions thereof, or a sequence with greater than 80%, greater than 85%, greater than 90%, or greater than 95% homology thereto. In some embodiments, the linker is SEQ ID NO:75, or conservative amino acid substitutions thereof, or a sequence with greater than 80%, greater than 85%, greater than 90%, or greater than 95% homology thereto. In some embodiments, the linker is SEQ ID NO:76, or conservative amino acid substitutions thereof, or a sequence with greater than 80%, greater than 85%, greater than 90%, or greater than 95% homology thereto.

Alternative linker sequences suitable for use in construction of scFv domains for the extracellular domain of CCRs are described in Bird, et al., Science 1988, 242, 423-426, the disclosures of which are incorporated by reference herein.

In an embodiment, a CCR of the present invention comprises a signal peptide. Without being bound by theory, a CCR comprising a signal peptide may, upon expression inside a cell, be directed to the endoplasmic reticulum and subsequently to the cell surface, where it is expressed. In an embodiment, the signal peptide may be at the amino terminus of the CCR. Any suitable signal peptide known in the art may be used with the CCRs of the present invention, such as those described in U.S. Pat. No. 9,856,322 and U.S. Patent Application Publication Nos. US 2019/0321404 A1, US 2019/0002573 A1, US 2020/0024342 A9, and US 2020/0078399 A1, the disclosures of which are incorporated by reference herein. Other suitable signal peptides are described elsewhere herein, including in the examples.

### 1. Extracellular PD-1 Domains

In an embodiment, a CCR of the present invention comprises an extracellular domain, wherein the extracellular domain comprises a PD-1 domain. In an embodiment, a CCR of the present invention comprises a fusion protein comprising an extracellular domain, a transmembrane domain, and an intracellular domain, wherein the extracellular domain is at least a portion of the extracellular domain of an inhibitory polypeptide, such as PD-1, that is associated with a negative signal that prevents activation of an immune response or induces apoptosis in a population of TILs, and wherein the intracellular domain is at least a portion of the intracellular domain of a stimulatory polypeptide that is associated with a positive signal that activates immune cells, such as CD28, and further wherein the fusion protein when displayed on said cell is able to switch said negative signal to said positive signal in the immune cell for conversion of a negative immune response to a positive immune response.

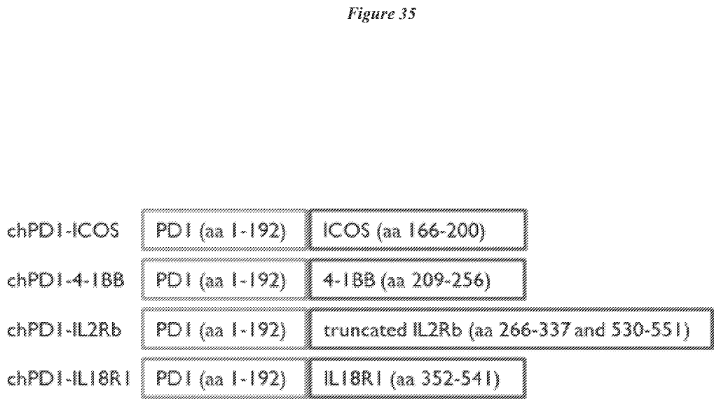

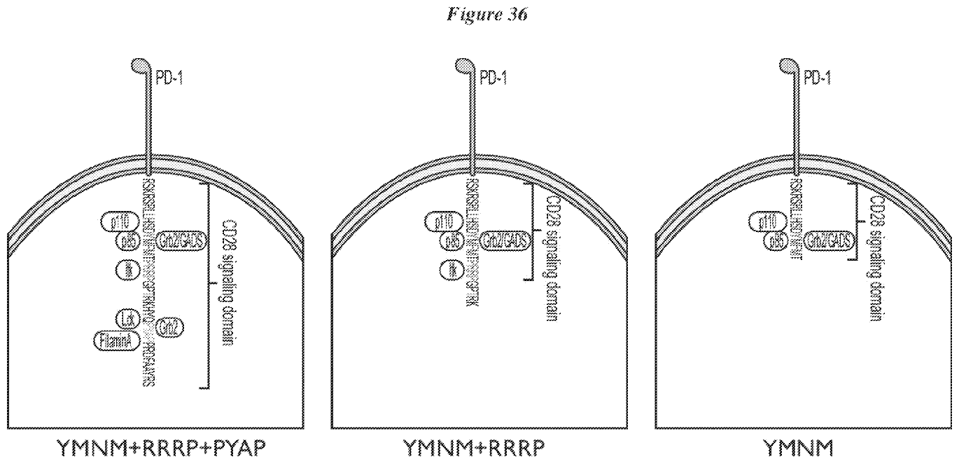

Amino acid sequences of exemplary PD-1 domains are provided in Table 35. Exemplary PD-1 CCR constructs using these domains are shown in FIG. 35. In an embodiment, a CCR of the present invention includes an extracellular PD-1 domain as shown in FIG. 35 or FIG. 36. PD-1 domains and CCR constructs using such domains, also referred to as PD-1 switch CCR constructs, including methods for their preparation, characterization, and use, are also described in U.S Patent Application Publication No. US 2019/0345219 A1, the disclosures of which are incorporated by reference herein. In an embodiment of the present invention, PD-1 switch CCR constructs are transduced into TILs during a Gen 2, Gen 3, or other TIL manufacturing process, including during a period between the pre-REP and REP stage of the Gen 2 process.

In an embodiment, a CCR of the present invention comprises an extracellular domain that includes SEQ ID NO:244, or conservative amino acid substitutions thereof. In an embodiment, a CCR of the present invention comprises an extracellular domain that includes SEQ ID NO:244, or a sequence with greater than 80%, greater than 85%, greater than 90%, greater than 95%, or greater than 98% homology thereto.

In an embodiment, a CCR of the present invention comprises an extracellular and transmembrane domain that includes SEQ ID NO:245, or conservative amino acid substitutions thereof. In an embodiment, a CCR of the present invention comprises an extracellular and transmembrane domain that includes SEQ ID NO:245, or a sequence with greater than 80%, greater than 85%, greater than 90%, greater than 95%, or greater than 98% homology thereto.

In an embodiment, a CCR of the present invention comprises an extracellular and transmembrane domain that includes SEQ ID NO:246, or conservative amino acid substitutions thereof. In an embodiment, a CCR of the present invention comprises an extracellular and transmembrane domain that includes SEQ ID NO:246, or a sequence with greater than 80%, greater than 85%, greater than 90%, greater than 95%, or greater than 98% homology thereto.

TABLE 35
Amino acid sequences of exemplary PD-1 extracellular domains.

| Identifier | Sequence (One-Letter Amino Acid Symbols) | |
|---|---|---|
| SEQ ID NO: 244 | MQIPQAPWPV VWAVLQLGWR PGWFLDSPDR PWNPPTFSPA LLVVTEGDNA TFTCSFSNTS | 60 |
| PD-1 | ESFVLNWYRM SPSNQTDKLA AFPEDRSQPG QDCRFRVTQL PNGRDFHMSV VRARRNDSGT | 120 |
| extracellular | YLCGAISLAP KAQIKESLRA ELRVTERRAE VPTAHPSPSP RPAGQFQTLV | 170 |
| domain | | |
| SEQ ID NO: 245 | MQIPQAPWPV VWAVLQLGWR PGWFLDSPDR PWNPPTFSPA LLVVTEGDNA TFTCSFSNTS | 60 |
| PD-1 | ESFVLNWYRM SPSNQTDKLA AFPEDRSQPG QDCRFRVTQL PNGRDFHMSV VRARRNDSGT | 120 |
| extracellular | YLCGAISLAP KAQIKESLRA ELRVTERRAE VPTAHPSPSP RPAGQFQTLV VGVVGGLLGS | 180 |
| and | LVLLVWVLAV I | 191 |
| transmembrane | | |
| domain | | |
| SEQ ID NO: 246 | MQIPQAPWPV VWAVLQLGWR PGWFLDSPDR PWNPPTFSPA LLVVTEGDNA TFTCSFSNTS | 60 |
| PD-1 | ESFVLNWYRM SPSNQTDKLA AFPEDRSQPG QDCRFRVTQL PNGRDFHMSV VRARRNDSGT | 120 |
| extracellular | YLCGAISLAP KAQIKESLRA ELRVTERRAE VPTAHPSPSP RPAGQFQTLV WVLVVVGGVL | 180 |
| domain and CD28 | ACYSLLVTVA FIIFWVF | 197 |
| transmembrane | | |
| domain | | |

Nucleotide sequences that encode exemplary PD-1 domains are provided in Table 36. In an embodiment, a CCR of the present invention comprises an extracellular and transmembrane domain, wherein the extracellular and transmembrane domain comprises a PD-1 domain encoded by the nucleotide sequence of SEQ ID NO:247. In an embodiment, a CCR of the present invention comprises an extracellular and transmembrane domain, wherein the extracellular and transmembrane domain is encoded by a nucleotide sequence that comprises a domain that retains at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% of the identity of SEQ ID NO:247. In an embodiment, including the foregoing embodiments, a nucleotide sequence in Table 36 is codon-optimized to improve protein expression.

In an embodiment, a CCR of the present invention comprises an extracellular and transmembrane domain, wherein the extracellular and transmembrane domain comprises a PD-1 and CD28 domain encoded by the nucleotide sequence of SEQ ID NO:248. In an embodiment, a CCR of the present invention comprises an extracellular and transmembrane domain, wherein the extracellular and transmembrane domain is encoded by a nucleotide sequence that comprises a domain that retains at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% of the identity of SEQ ID NO:248. In an embodiment, including the foregoing embodiments, a nucleotide sequence in Table 36 is codon-optimized to improve protein expression.

TABLE 36
Nucleotide sequences of selected exemplary extracellular PD-1 domains.

| Identifier | Sequence (One-Letter Nucleotide Symbols) | |
|---|---|---|
| SEQ ID NO: 247 | GCTCACCTCC GCCTGAGCAG TGGAGAAGGC GGCACTCTGG TGGGGCTGCT CCAGGCATGC | 60 |
| PDCD1 | AGATCCCACA GGCGCCCTGG CCAGTCGTCT GGGCGGTGCT ACAACTGGGC TGGCGGCCAG | 120 |
| extracellular | GATGGTTCTT AGACTCCCCA GACAGGCCCT GGAACCCCCC CACCTTCTCC CCAGCCCTGC | 180 |
| and PDCD1 | TCGTGGTGAC CGAAGGGGAC AACGCCACCT TCACCTGCAG CTTCTCCAAC ACATCGGAGA | 240 |
| transmembrane | GCTTCGTGCT AAACTGGTAC CGCATGAGCC CCAGCAACCA GACGGACAAG CTGGCCGCCT | 300 |
| domain | TCCCCGAGGA CCGCAGCCAG CCCGGCCAGG ACTGCCGCTT CCGTGTCACA CAACTGCCCA | 360 |
| | ACGGGCGTGA CTTCCACATG AGCGTGGTCA GGGCCCGGCG CAATGACAGC GGCACCTACC | 420 |
| | TCTGTGGGGC CATCTCCCTG GCCCCCAAGG CGCAGATCAA AGAGAGCCTG CGGGCAGAGC | 480 |
| | TCAGGGTGAC AGAGAGAAGG GCAGAAGTGC CCACAGCCCA CCCCAGCCCC TCACCCAGGC | 540 |
| | CAGCCGGCCA GTTCCAAACC CTGGTGGTTG GTGTCGTGGG CGGCCTGCTG GGCAGCCTGG | 600 |
| | TGCTGCTAGT CTGGGTCCTG GCCGTCATC | 629 |
| SEQ ID NO: 248 | GCTCACCTCC GCCTGAGCAG TGGAGAAGGC GGCACTCTGG TGGGGCTGCT CCAGGCATGC | 60 |
| PD-1 | AGATCCCACA GGCGCCCTGG CCAGTCGTCT GGGCGGTGCT ACAACTGGGC TGGCGGCCAG | 120 |
| extracellular | GATGGTTCTT AGACTCCCCA GACAGGCCCT GGAACCCCCC CACCTTCTCC CCAGCCCTGC | 180 |
| and CD28 | TCGTGGTGAC CGAAGGGGAC AACGCCACCT TCACCTGCAG CTTCTCCAAC ACATCGGAGA | 240 |
| transmembrane | GCTTCGTGCT AAACTGGTAC CGCATGAGCC CCAGCAACCA GACGGACAAG CTGGCCGCCT | 300 |
| domain | TCCCCGAGGA CCGCAGCCAG CCCGGCCAGG ACTGCCGCTT CCGTGTCACA CAACTGCCCA | 360 |
| | ACGGGCGTGA CTTCCACATG AGCGTGGTCA GGGCCCGGCG CAATGACAGC GGCACCTACC | 420 |
| | TCTGTGGGGC CATCTCCCTG GCCCCCAAGG CGCAGATCAA AGAGAGCCTG CGGGCAGAGC | 480 |
| | TCAGGGTGAC AGAGAGAAGG GCAGAAGTGC CCACAGCCCA CCCCAGCCCC TCACCCAGGC | 540 |
| | CAGCCGGCCA GTTCCAAACC CTGGTGTTTT GGGTGCTGGT GGTGGTTGGT GGAGTCCTGG | 600 |
| | CTTGCTATAG CTTGCTAGTA ACAGTGGCCT TTATTATTTT CTGGGTG | 647 |

In an embodiment, a CCR of the present invention comprises a PD-1 switch construct as described in Liu, et al., Cancer Res. 2016, 76, 1578-90, the disclosures of which are incorporated by reference herein. In an embodiment, a CCR of the present invention comprises a PD-1 switch construct as described in International Patent Application Publication No. WO 2018/119298 A1, the disclosures of which are incorporated by reference herein.

### 2. Extracellular PD-L1 Binding Domains

In an embodiment, a CCR of the present invention comprises an extracellular domain, wherein the extracellular domain comprises a PD-L1 binding scFv domain. In an embodiment, a CCR of the present invention comprises an extracellular anti-PD-L1 domain comprising a V_{H} domain and a V_{L} domain. In an embodiment, a CCR of the present invention comprises a construct as shown in FIG. 34, wherein the V_{H} and V_{L} domains are anti-PD-L1 V_{H} and V_{L} domains, and the linker is as described herein. In an embodiment, an anti-PD-L1 scFv domain includes the scFv antibodies 38A1 and 19H9, the properties and preparation of which, including the nucleotide sequences encoding such antibodies, are described in U.S. Patent Application Publication No. US 2019/0298770 A1, the disclosures of which are incorporated by reference herein. The amino acid sequences of exemplary anti-PD-L1 binding scFv domains are provided in Table 37. In an embodiment, a CCR of the present invention comprises a construct as shown in FIG. 34, wherein the V_{H} and V_{L} domains are anti-PD-L1 V_{H} and V_{L} domains, wherein the V domain is selected from the group consisting of SEQ ID NO:250, SEQ ID NO:259, and conservative amino acid substitutions thereof, and wherein the V_{L} domain is selected from the group consisting of SEQ ID NO:251, SEQ ID NO:260, and conservative amino acid substitutions thereof.

TABLE 37
Amino acid sequences of exemplary extracellular PD-L1 binding domains.

| Identifier | Sequence (One-Letter Amino Acid Symbols) | |
|---|---|---|
| SEQ ID NO: 249 | EVQLVESGGG LVQPGGSLRL SCAASGFTFS NYAMSWVRQA PGKGLEWVST ISGSGGTTYY | 60 |
| SCFV-Fc | ADSVKGRFTI SRDNSKNTLY LQMNSLRVED TAVYYCAKDW FRSSSPDAFD IWGQGTTVTV | 120 |
| antibody 38A1 | SAGGGGSGGG GSGGGGSGAP SYVLTQPPSV SVAPGQTARI TCGGNNIGRK IVHWYQQRPG | 180 |
| | QAPVLVIYYD TDRPAGIPER FSGSNSGNMA TITISTVGAG DEADYYCQVW DTGSDHVVFG | 240 |
| | GGTKLTVL | 248 |
| SEQ ID NO: 250 | EVQLVESGGG LVQPGGSLRL SCAASGFTFS NYAMSWVRQA PGKGLEWVST ISGSGGTTYY | 60 |
| scFv antibody | ADSVKGRFTI SRDNSKNTLY LQMNSLRVED TAVYYCAKDW FRSSSPDAFD IWGQGTTVTV | 120 |
| 38A1 variable | SA | 122 |
| heavy chain | | |
| SEQ ID NO: 251 | SYVLTQPPSV SVAPGQTARI TCGGNNIGRK IVHWYQQRPG QAPVLVIYYD TDRPAGIPER | 60 |
| scFv antibody | FSGSNSGNMA TLTISTVGAG DEADYYCQVW DTGSDHVVFG GGTKLTVL | 108 |
| 38A1 variable | | |
| light chain | | |
| SEQ ID NO: 252 | GFTFSNYA | 8 |
| 38A1 anti-PD-L1 | | |
| heavy chain | | |
| CDR1 | | |
| SEQ ID NO: 253 | ISGSGGTT | 8 |
| 38A1 anti-PD-L1 | | |
| heavy chain | | |
| CDR2 | | |
| SEQ ID NO: 254 | AKDWFRSSSP DAFDE | 15 |
| 38A1 anti-PD-L1 | | |
| heavy chain | | |
| CDR3 | | |
| SEQ ID NO: 255 | NIGRKI | 6 |
| 38Al anti-PD-L1 | | |
| light chain | | |
| CDR1 | | |
| SEQ ID NO: 256 | YDT | 3 |
| 38A1 anti-PD-L1 | | |
| light chain | | |
| CDR2 | | |
| SEQ ID NO: 257 | QVWDTGSDHV V | 11 |
| 38A1 anti-PD-L1 | | |
| light chain | | |
| CDR3 | | |
| SEQ ID NO: 258 | QVQLQESGGG LVKPGGSLRL SCAASGFTFS SYSMNWVRQA PGKGLEWVSG INTAGDTHYP | 60 |
| SCFv-Fc | ESVKGRFTIS RDNARNSLNL QMNSLRAEDT AVYYCVRERV EREYSGYDAF DIWGQGTTVT | 120 |
| antibody 19H9 | VSAGGGGSGG GGSGGGGSGA PNFMLTQPHS VSESLGKTVT ISCTGSSGSI ARKFVQWYQQ | 180 |
| | RPGSSPTTVI YENNQRPSGV SDRFSGSIGS SSNSASLTIS GLKTEDEADY YCQSYDSSNV | 240 |
| | VFGGGTKVTV L | 251 |
| SEQ ID NO: 259 | QVQLQESGGG LVKPGGSLRL SCAASGFTFS SYSMNWVRQA PGKGLEWVSG INTAGDTHYP | 60 |
| scFv antibody | ESVKGRFTIS RDNARNSINL QMNSLRAEDT AVYYCVRERV EREYSGYDAF DIWGQGTTVT | 120 |
| 19H9 variable | VSA | 123 |
| heavy chain | | |
| SEQ ID NO: 260 | NFMITQPHSV SESLGKTVTI SCTGSSGSIA RKFVQWYQQR PGSSPTTVIY ENNQRPSGVS | 60 |
| scFv antibody | DRFSGSIGSS SNSASLTISG LKTEDEADYY CQSYDSSNVV FGGGTKVTVL | 110 |
| 19H9 variable | | |
| light chain | | |
| SEQ ID NO: 261 | GFTFSSYS | 8 |
| 19H9 anti-PD-L1 | | |
| heavy chain | | |
| CDR1 | | |
| SEQ ID NO: 262 | INTAGDT | 7 |
| 19H9 anti-PD-L1 | | |
| heavy chain | | |
| CDR2 | | |
| SEQ ID NO: 263 | VRERVEREYS GYDAFDI | 17 |
| 19H9 anti-PD-L1 | | |
| heavy chain | | |
| CDR3 | | |
| SEQ ID NO: 264 | SGSIARKF | 8 |
| 19H9 anti-PD-L1 | | |
| light chain | | |
| CDR1 | | |
| SEQ ID NO: 265 | ENN | 3 |
| 19H9 anti-PD-L1 | | |
| light chain | | |
| CDR2 | | |
| SEQ ID NO: 266 | QSYDSSNVV | 9 |
| 19H9 anti-PD-L1 | | |
| light chain | | |
| CDR3 | | |

In an embodiment, an anti-PD-L1 scFv domain comprises the sequence shown in SEQ ID NO:249, or conservative amino acid substitutions thereof. In an embodiment, an anti-PD-L1 scFv domain comprises the scFv antibody 38A1, or conservative amino acid substitutions thereof. In an embodiment, an anti-PD-L1 scFv domain comprises a scFv domain that is at least 99% identical to the sequence shown in SEQ ID NO:249. In an embodiment, an anti-PD-L1 scFv domain comprises a scFv domain that is at least 98% identical to the sequence shown in SEQ ID NO:249. In an embodiment, an anti-PD-L1 scFv domain comprises a scFv domain that is at least 97% identical to the sequence shown in SEQ ID NO:249. In an embodiment, an anti-PD-L1 scFv domain comprises a scFv domain that is at least 96% identical to the sequence shown in SEQ ID NO:249. In an embodiment, an anti-PD-L1 scFv domain comprises a scFv domain that is at least 95% identical to the sequence shown in SEQ ID NO:249. In an embodiment, an anti-PD-L1 scFv domain comprises a scFv domain that is at least 90% identical to the sequence shown in SEQ ID NO:249. In an embodiment, an anti-PD-L1 scFv domain comprises a scFv domain that is at least 85% identical to the sequence shown in SEQ ID NO:249. In an embodiment, an anti-PD-L1 scFv domain comprises a scFv domain that is at least 80% identical to the sequence shown in SEQ ID NO:249.

In an embodiment, an anti-PD-L1 scFv domain comprises a heavy chain variable region (V_{H}) domain and a light chain variable region (V_{L}) domain, wherein the V_{H} domain comprises the sequence shown in SEQ ID NO:250, or conservative amino acid substitutions thereof, and the light chain variable region (V_{L}) comprises the sequence shown in SEQ ID NO:251, or conservative amino acid substitutions thereof. In an embodiment, an anti-PD-L1 scFv domain comprises V_{H} and V_{L} regions that are each at least 99% identical to the sequences shown in SEQ ID NO:250 and SEQ ID NO:251, respectively. In an embodiment, an anti-PD-L1 scFv domain comprises V_{H} and V_{L} regions that are each at least 98% identical to the sequences shown in SEQ ID NO:250 and SEQ ID NO:251, respectively. In an embodiment, an anti-PD-L1 scFv domain comprises V_{H} and V_{L} regions that are each at least 97% identical to the sequences shown in SEQ ID NO:250 and SEQ ID NO:251, respectively. In an embodiment, an anti-PD-L1 scFv domain comprises V_{H} and V_{L} regions that are each at least 96% identical to the sequences shown in SEQ ID NO:250 and SEQ ID NO:251, respectively. In an embodiment, an anti-PD-L1 scFv domain comprises V_{H} and V_{L} regions that are each at least 95% identical to the sequences shown in SEQ ID NO:250 and SEQ ID NO:251, respectively. In an embodiment, an anti-PD-L1 scFv domain comprises V_{H} and V_{L} regions that are each at least 90% identical to the sequences shown in SEQ ID NO:250 and SEQ ID NO:251, respectively. In an embodiment, an anti-PD-L1 scFv domain comprises V_{H} and V_{L} regions that are each at least 85% identical to the sequences shown in SEQ ID NO:250 and SEQ ID NO:251, respectively. In an embodiment, an anti-PD-L1 scFv domain comprises V_{H} and V_{L} regions that are each at least 80% identical to the sequences shown in SEQ ID NO:250 and SEQ ID NO:251, respectively.

In an embodiment, an anti-PD-L1 scFv domain comprises the heavy chain CDR1, CDR2 and CDR3 domains having the sequences set forth in SEQ ID NO:252, SEQ ID NO:253, and/or SEQ ID NO:254, respectively, or conservative amino acid substitutions thereof, and/or light chain CDR1, CDR2 and CDR3 domains having the sequences set forth in SEQ ID NO:255, SEQ ID NO:256, and/or SEQ ID NO:257, respectively, or conservative amino acid substitutions thereof.

In an embodiment, an anti-PD-L1 scFv domain comprises the sequence shown in SEQ ID NO:258, or conservative amino acid substitutions thereof. In an embodiment, an anti-PD-L1 scFv domain comprises the scFv antibody 19H9, or conservative amino acid substitutions thereof. In an embodiment, an anti-PD-L1 scFv domain comprises a scFv domain that is at least 99% identical to the sequence shown in SEQ ID NO:258. In an embodiment, an anti-PD-L1 scFv domain comprises a scFv domain that is at least 98% identical to the sequence shown in SEQ ID NO:258. In an embodiment, an anti-PD-L1 scFv domain comprises a scFv domain that is at least 97% identical to the sequence shown in SEQ ID NO:258. In an embodiment, an anti-PD-L1 scFv domain comprises a scFv domain that is at least 96% identical to the sequence shown in SEQ ID NO:258. In an embodiment, an anti-PD-L1 scFv domain comprises a scFv domain that is at least 95% identical to the sequence shown in SEQ ID NO:258. In an embodiment, an anti-PD-L1 scFv domain comprises a scFv domain that is at least 90% identical to the sequence shown in SEQ ID NO:258. In an embodiment, an anti-PD-L1 scFv domain comprises a scFv domain that is at least 85% identical to the sequence shown in SEQ ID NO:258. In an embodiment, an anti-PD-L1 scFv domain comprises a scFv domain that is at least 80% identical to the sequence shown in SEQ ID NO:258.

In an embodiment, an anti-PD-L1 scFv domain comprises a heavy chain variable region (V_{H}) domain and a light chain variable region (V_{L}) domain, wherein the V_{H} domain comprises the sequence shown in SEQ ID NO:259, or conservative amino acid substitutions thereof, and the light chain variable region (V_{L}) comprises the sequence shown in SEQ ID NO:260, or conservative amino acid substitutions thereof. In an embodiment, an anti-PD-L1 scFv domain comprises V_{H} and V_{L} regions that are each at least 99% identical to the sequences shown in SEQ ID NO:259 and SEQ ID NO:260, respectively. In an embodiment, an anti-PD-L1 scFv domain comprises V_{H} and V_{L} regions that are each at least 98% identical to the sequences shown in SEQ ID NO:259 and SEQ ID NO:260, respectively. In an embodiment, an anti-PD-L1 scFv domain comprises V_{H} and V_{L} regions that are each at least 97% identical to the sequences shown in SEQ ID NO:259 and SEQ ID NO:260, respectively. In an embodiment, an anti-PD-L1 scFv domain comprises V_{H} and V_{L} regions that are each at least 96% identical to the sequences shown in SEQ ID NO:259 and SEQ ID NO:260, respectively. In an embodiment, an anti-PD-L1 scFv domain comprises V_{H} and V_{L} regions that are each at least 95% identical to the sequences shown in SEQ ID NO:259 and SEQ ID NO:260, respectively. In an embodiment, an anti-PD-L1 scFv domain comprises V_{H} and V_{L} regions that are each at least 90% identical to the sequences shown in SEQ ID NO:259 and SEQ ID NO:260, respectively. In an embodiment, an anti-PD-L1 scFv domain comprises V_{H} and V_{L} regions that are each at least 85% identical to the sequences shown in SEQ ID NO:259 and SEQ ID NO:260, respectively. In an embodiment, an anti-PD-L1 scFv domain comprises V_{H} and V_{L} regions that are each at least 80% identical to the sequences shown in SEQ ID NO:259 and SEQ ID NO:260, respectively.

In an embodiment, an anti-PD-L1 scFv domain comprises the heavy chain CDR1, CDR2 and CDR3 domains having the sequences set forth in SEQ ID NO:261, SEQ ID NO:262, and/or SEQ ID NO:263, respectively, or conservative amino acid substitutions thereof, and/or light chain CDR1, CDR2 and CDR3 domains having the sequences set forth in SEQ ID NO:264, SEQ ID NO:265, and/or SEQ ID NO:266, respectively, or conservative amino acid substitutions thereof.

In an embodiment, an anti-PD-L1 scFv domain comprises a heavy chain variable region (V_{H}) domain and a light chain variable region (V_{L}) domain of durvalumab. In an embodiment, an anti-PD-L1 scFv domain comprises a V_{H} domain and a V_{L} domain, wherein the V_{H} domain comprises the sequence shown in SEQ ID NO:180, or conservative amino acid substitutions thereof, and the light chain variable region (V_{L}) comprises the sequence shown in SEQ ID NO:181, or conservative amino acid substitutions thereof. In an embodiment, an anti-PD-L1 scFv domain comprises V_{H} and V_{L} regions that are each at least 99% identical to the sequences shown in SEQ ID NO:180 and SEQ ID NO:181, respectively. In an embodiment, an anti-PD-L1 scFv domain comprises V_{H} and V_{L} regions that are each at least 98% identical to the sequences shown in SEQ ID NO:180 and SEQ ID NO:181, respectively. In an embodiment, an anti-PD-L1 scFv domain comprises V_{H} and V_{L} regions that are each at least 97% identical to the sequences shown in SEQ ID NO:180 and SEQ ID NO:181, respectively. In an embodiment, an anti-PD-L1 scFv domain comprises V_{H} and V_{L} regions that are each at least 96% identical to the sequences shown in SEQ ID NO:180 and SEQ ID NO:181, respectively. In an embodiment, an anti-PD-L1 scFv domain comprises V_{H} and V_{L} regions that are each at least 95% identical to the sequences shown in SEQ ID NO:180 and SEQ ID NO:181, respectively. In an embodiment, an anti-PD-L1 scFv domain comprises V_{H} and V_{L} regions that are each at least 90% identical to the sequences shown in SEQ ID NO:180 and SEQ ID NO:181, respectively. In an embodiment, an anti-PD-L1 scFv domain comprises V_{H} and V_{L} regions that are each at least 85% identical to the sequences shown in SEQ ID NO:180 and SEQ ID NO:181, respectively. In an embodiment, an anti-PD-L1 scFv domain comprises V_{H} and V_{L} regions that are each at least 80% identical to the sequences shown in SEQ ID NO:180 and SEQ ID NO:181, respectively.

In an embodiment, an anti-PD-L1 scFv domain comprises the heavy chain CDR1, CDR2 and CDR3 domains having the sequences set forth in SEQ ID NO:182, SEQ ID NO:183, and/or SEQ ID NO:184, respectively, or conservative amino acid substitutions thereof, and/or light chain CDR1, CDR2 and CDR3 domains having the sequences set forth in SEQ ID NO:185, SEQ ID NO:186, and/or SEQ ID NO:187, respectively, or conservative amino acid substitutions thereof.

In an embodiment, an anti-PD-L1 scFv domain comprises a heavy chain variable region (V_{H}) domain and a light chain variable region (V_{L}) domain of avelumab. In an embodiment, an anti-PD-L1 scFv domain comprises a V_{H} domain and a V_{L} domain, wherein the V_{H} domain comprises the sequence shown in SEQ ID NO:190, or conservative amino acid substitutions thereof, and the light chain variable region (V_{L}) comprises the sequence shown in SEQ ID NO:191, or conservative amino acid substitutions thereof. In an embodiment, an anti-PD-L1 scFv domain comprises V_{H} and V_{L} regions that are each at least 99% identical to the sequences shown in SEQ ID NO:190 and SEQ ID NO:191, respectively. In an embodiment, an anti-PD-L1 scFv domain comprises V_{H} and V_{L} regions that are each at least 98% identical to the sequences shown in SEQ ID NO:190 and SEQ ID NO:191, respectively. In an embodiment, an anti-PD-L1 scFv domain comprises V_{H} and V_{L} regions that are each at least 97% identical to the sequences shown in SEQ ID NO:190 and SEQ ID NO:191, respectively. In an embodiment, an anti-PD-L1 scFv domain comprises V_{H} and V_{L} regions that are each at least 96% identical to the sequences shown in SEQ ID NO:190 and SEQ ID NO:191, respectively. In an embodiment, an anti-PD-L1 scFv domain comprises V_{H} and V_{L} regions that are each at least 95% identical to the sequences shown in SEQ ID NO:190 and SEQ ID NO:191, respectively. In an embodiment, an anti-PD-L1 scFv domain comprises V_{H} and V_{L} regions that are each at least 90% identical to the sequences shown in SEQ ID NO:190 and SEQ ID NO:191, respectively. In an embodiment, an anti-PD-L1 scFv domain comprises V_{H} and V_{L} regions that are each at least 85% identical to the sequences shown in SEQ ID NO:190 and SEQ ID NO:191, respectively. In an embodiment, an anti-PD-L1 scFv domain comprises V_{H} and V_{L} regions that are each at least 80% identical to the sequences shown in SEQ ID NO:190 and SEQ ID NO:191, respectively.

In an embodiment, an anti-PD-L1 scFv domain comprises the heavy chain CDR1, CDR2 and CDR3 domains having the sequences set forth in SEQ ID NO:192, SEQ ID NO:193, and/or SEQ ID NO:194, respectively, or conservative amino acid substitutions thereof, and/or light chain CDR1, CDR2 and CDR3 domains having the sequences set forth in SEQ ID NO:195, SEQ ID NO:196, and/or SEQ ID NO:197, respectively, or conservative amino acid substitutions thereof.

In an embodiment, an anti-PD-L1 scFv domain comprises a heavy chain variable region (V_{H}) domain and a light chain variable region (V_{L}) domain of atezolizumab. In an embodiment, an anti-PD-L1 scFv domain comprises a V_{H} domain and a V_{L} domain, wherein the V_{H} domain comprises the sequence shown in SEQ ID NO:200, or conservative amino acid substitutions thereof, and the light chain variable region (V_{L}) comprises the sequence shown in SEQ ID NO:201, or conservative amino acid substitutions thereof. In an embodiment, an anti-PD-L1 scFv domain comprises V_{H} and V_{L} regions that are each at least 99% identical to the sequences shown in SEQ ID NO:200 and SEQ ID NO:201, respectively. In an embodiment, an anti-PD-L1 scFv domain comprises V_{H} and V_{L} regions that are each at least 98% identical to the sequences shown in SEQ ID NO:200 and SEQ ID NO:201, respectively. In an embodiment, an anti-PD-L1 scFv domain comprises V_{H} and V_{L} regions that are each at least 97% identical to the sequences shown in SEQ ID NO:200 and SEQ ID NO:201, respectively. In an embodiment, an anti-PD-L1 scFv domain comprises V_{H} and V_{L} regions that are each at least 96% identical to the sequences shown in SEQ ID NO:200 and SEQ ID NO:201, respectively. In an embodiment, an anti-PD-L1 scFv domain comprises V_{H} and V_{L} regions that are each at least 95% identical to the sequences shown in SEQ ID NO:200 and SEQ ID NO:201, respectively. In an embodiment, an anti-PD-L1 scFv domain comprises V_{H} and V_{L} regions that are each at least 90% identical to the sequences shown in SEQ ID NO:200 and SEQ ID NO:201, respectively. In an embodiment, an anti-PD-L1 scFv domain comprises V_{H} and V_{L} regions that are each at least 85% identical to the sequences shown in SEQ ID NO:200 and SEQ ID NO:201, respectively. In an embodiment, an anti-PD-L1 scFv domain comprises V_{H} and V_{L} regions that are each at least 80% identical to the sequences shown in SEQ ID NO:200 and SEQ ID NO:201, respectively.

In an embodiment, an anti-PD-L1 scFv domain comprises the heavy chain CDR1, CDR2 and CDR3 domains having the sequences set forth in SEQ ID NO:202, SEQ ID NO:203, and/or SEQ ID NO:204, respectively, or conservative amino acid substitutions thereof, and/or light chain CDR1, CDR2 and CDR3 domains having the sequences set forth in SEQ ID NO:205, SEQ ID NO:206, and/or SEQ ID NO:207, respectively, or conservative amino acid substitutions thereof.

In an embodiment, the anti-PD-L1 binding domain includes an scFv, a V_{H} and/or V_{L} sequence, or a heavy chain and/or a light chain CDR1, CDR2, and/or CDR3 sequence, or conservative amino acid substitutions thereof, or nucleotides that encode such sequences, as disclosed in U.S. Patent Application Publication No. US 2019/0048085 A1, the disclosures of which are incorporated by reference herein.

In an embodiment, the anti-PD-L1 binding domain includes an scFv, a V_{H} and/or V_{L} sequence, or a heavy chain and/or a light chain CDR1, CDR2, and/or CDR3 sequence, or conservative amino acid substitutions thereof, or nucleotides that encode such sequences, as disclosed in U.S. Pat. No. 10,604,581, the disclosures of which are incorporated by reference herein.

### 3. Extracellular CEA Binding Domains

In an embodiment, a CCR of the present invention comprises an extracellular domain, wherein the extracellular domain comprises a carcinomebryonic antigen (CEA) binding domain, also referred to herein as an anti-CEA domain. In an embodiment, a CCR of the present invention comprises an extracellular domain, wherein the extracellular domain comprises a CD66 binding domain. In an embodiment, a CCR of the present invention comprises an extracellular domain, wherein the extracellular domain comprises a CD66 binding domain selected from the group consisting of a CD66a binding domain, a CD66b binding domain, a CD66c binding domain, a CD66d binding domain, a CD66e binding domain, and a CD66f binding domain. In an embodiment, the CEA or CD66 binding domain is an scFv domain. In an embodiment, the CEA binding domain binds to murine CEA. In an embodiment, the CEA binding domain binds to human CEA. In an embodiment, a CCR of the present invention comprises a construct as shown in FIG. 34, wherein the V_{H} and V_{L} domains are anti-CEA V_{H} and V_{L} domains, and the linker is as described herein. In an embodiment, the CEA binding domain includes scFv antibodies prepared from the CDR, V_{H}, and V_{L} domains described in U.S. Pat. No. 8,470,994, the disclosures of which are incorporated by reference herein. The amino acid sequences of exemplary CEA binding scFv domains are provided in Table 38.

TABLE 38
Amino acid sequences of exemplary extracellular CEA binding domains.

| Identifier | Sequence (One-Letter Amino Acid Symbols) | |
|---|---|---|
| SEQ ID NO: 267 | QVQLVESGGG VVQPGRSLRL SCSSSGFALT DYYMSWVRQA PGKGLEWLGE IANKANGHTT | 60 |
| Anti-CEA | DYSPSVKGRF TISRDNSKNT LFLQMDSLRP EDTGVYFCAR DMGERWNFDV WGQGTPVTVS | 120 |
| variable heavy | S | 121 |
| chain | | |
| SEQ ID NO: 268 | DIQLTQSPSS LSASVGDRVT MTCSASSRVS YIHWYQQKPG KAPKRWIYGT STLASGVPAR | 60 |
| Anti-CEA | FSGSGSGTDF TFTISSLQPE DIATYYCQQW SYNPPTFGQG TKVEIKR | 107 |
| variable light | | |
| chain | | |
| SEQ ID NO: 269 | DYYMS | 5 |
| Anti-CEA heavy | | |
| chain CDR1 | | |
| SEQ ID NO: 270 | FIANKANGET TDYSPSVKG | 19 |
| Anti-CEA heavy | | |
| chain CDR2 | | |
| SEQ ID NO: 271 | DMGIRWNFDV | 10 |
| Anti-CEA heavy | | |
| chain CDR3 | | |
| SEQ ID NO: 272 | SASSRVSYIH | 10 |
| Anti-CEA light | | |
| chain CDR1 | | |
| SEQ ID NO: 273 | GTSTLAS | 7 |
| Anti-CEA light | | |
| chain CDR2 | | |
| SEQ ID NO: 274 | QQWSYNPPT | 9 |
| Anti-CEA light | | |
| chain CDR3 | | |

In an embodiment, an anti-CEA scFv domain comprises a V_{H} domain and a V_{L} domain, wherein the V_{H} domain comprises the sequence shown in SEQ ID NO:267, or conservative amino acid substitutions thereof, and the light chain variable region (V_{L}) comprises the sequence shown in SEQ ID NO:268, or conservative amino acid substitutions thereof. In an embodiment, an anti-CEA scFv domain comprises V_{H} and V_{L} regions that are each at least 99% identical to the sequences shown in SEQ ID NO:267 and SEQ ID NO:268, respectively. In an embodiment, an anti-CEA scFv domain comprises V_{H} and V_{L} regions that are each at least 98% identical to the sequences shown in SEQ ID NO:267 and SEQ ID NO:268, respectively. In an embodiment, an anti-CEA scFv domain comprises V_{H} and V_{L} regions that are each at least 97% identical to the sequences shown in SEQ ID NO:267 and SEQ ID NO:268, respectively. In an embodiment, an anti-CEA scFv domain comprises V_{H} and V_{L} regions that are each at least 96% identical to the sequences shown in SEQ ID NO:267 and SEQ ID NO:268, respectively. In an embodiment, an anti-CEA scFv domain comprises V_{H} and V_{L} regions that are each at least 95% identical to the sequences shown in SEQ ID NO:267 and SEQ ID NO:268, respectively. In an embodiment, an anti-CEA scFv domain comprises V_{H} and V_{L} regions that are each at least 90% identical to the sequences shown in SEQ ID NO:267 and SEQ ID NO:268, respectively. In an embodiment, an anti-CEA scFv domain comprises V_{H} and V_{L} regions that are each at least 85% identical to the sequences shown in SEQ ID NO:267 and SEQ ID NO:268, respectively. In an embodiment, an anti-CEA scFv domain comprises V_{H} and V_{L} regions that are each at least 80% identical to the sequences shown in SEQ ID NO:267 and SEQ ID NO:268, respectively.

In an embodiment, an anti-CEA scFv domain comprises the heavy chain CDR1, CDR2 and CDR3 domains having the sequences set forth in SEQ ID NO:269, SEQ ID NO:270, and/or SEQ ID NO:271, respectively, or conservative amino acid substitutions thereof, and/or light chain CDR1, CDR2 and CDR3 domains having the sequences set forth in SEQ ID NO:272, SEQ ID NO:273, and/or SEQ ID NO:274, respectively, or conservative amino acid substitutions thereof.

In an embodiment, the anti-CEA binding domain includes an scFv, a V_{H} and/or V_{L} sequence, or a heavy chain and/or a light chain CDR1, CDR2, and/or CDR3 sequence, or variants, fragments, or derivatives thereof, or conservative amino acid substitutions thereof, or nucleotides that encode such sequences, as disclosed in International Patent Application Publication No. WO 2020/152451 A1, the disclosures of which are incorporated by reference herein.

In an embodiment, the anti-CEA binding domain includes an scFv, a V_{H} and/or V_{L} sequence, or a heavy chain and/or a light chain CDR1, CDR2, and/or CDR3 sequence, or variants, fragments, or derivatives thereof, or conservative amino acid substitutions thereof, or nucleotides that encode such sequences, as disclosed in U.S. Patent Application Publication No. US 2009/0117108 A1, the disclosures of which are incorporated by reference herein.

In an embodiment, the anti-CEA binding domain includes an scFv, a V_{H} and/or V_{L} sequence, or a heavy chain and/or a light chain CDR1, CDR2, and/or CDR3 sequence, or variants, fragments, or derivatives thereof, or conservative amino acid substitutions thereof, or nucleotides that encode such sequences, as disclosed in U.S. Pat. No. 5,081,235, the disclosures of which are incorporated by reference herein.

In an embodiment, the anti-CEA binding domain includes an scFv, a V_{H} and/or V_{L} sequence, or a heavy chain and/or a light chain CDR1, CDR2, and/or CDR3 sequence, or variants, fragments, or derivatives thereof, or conservative amino acid substitutions thereof, as disclosed in U.S. Pat. No. 10,865,243, the disclosures of which are incorporated by reference herein.

### 4. Extracellular CD73 Binding Domains

In an embodiment, a CCR of the present invention comprises an extracellular domain, wherein the extracellular domain comprises a CD73 binding domain, also referred to herein as an anti-CD73 domain. CD73, also known as ecto-5′-nucleotidase or ecto-5′NT is a glycosyl-phosphatidylinositol (GPI)-linked cell surface enzyme expressed in endothelial cells and subsets of hematopoietic cells. Resta, et al., Immunol. Rev. 1998, 161, 95-109. CD73 is known to catalyze the dephosphorylation of extracellular nucleoside monophosphates into nucleosides, such as adenosine, which has been shown to regulate proliferation and migration of many cancers and to have an immunosuppressive effect through the regulation of anti-tumor T cells. Zhang, et al., Cancer Res. 2010, 70, 6407-11). CD73 is expressed on be expressed on many different cancers, including colon, lung, pancreas, ovary, bladder, leukemia, glioma, glioblastoma, melanoma, thyroid, esophageal, prostate and breast cancers. Jin, et al., Cancer Res. 2010, 70, 2245-55; Stagg, et al., Proc. Nat'l. Acad. Sci. 2010, 107, 1547-52. Moreover, CD73 expression in cancer has been linked to increased proliferation, migration, neovascularization, invasiveness, metastasis, and shorter patient survival. In an embodiment, the CD73 binding domain is an scFv domain. In an embodiment, the CD73 binding domain binds to murine CD73. In an embodiment, the CD73 binding domain binds to human CD73. In an embodiment, a CCR of the present invention comprises a construct as shown in FIG. 34, wherein the V_{H} and V_{L} domains are anti-CD73 V_{H} and V_{L} domains, and the linker is as described herein.

In an embodiment, the anti-CD73 binding domain includes a V_{H} and/or V_{L} sequence or a heavy chain and/or a light chain CDR1, CDR2, and/or CDR3 sequence, or nucleotides that encode such sequences, as disclosed in U.S. Pat. Nos. 10,287,362; 10,556,968; and 10,864,269; the disclosures of which are incorporated by reference herein. The amino acid sequences of exemplary CD73 binding scFv domains are provided in Table 39.

TABLE 39
Amino acid sequences of exemplary extracellular CD73 binding domains.

| Identifier | Sequence (One-Letter Amino Acid Symbols) | |
|---|---|---|
| SEQ ID NO: 275 | EVQLLESGGG LVQPGGSLRL SCAASGFTFS SYAYSWVRQA PGKGLEWVSA ISGSGGRTYY | 60 |
| Anti-CD73 | ADSVKGRFTI SRDNSKNTLY LQMNSLRAED TAVYYCARLG YGRVDEWGRG TLVTVSS | 117 |
| variable heavy | | |
| chain | | |
| SEQ ID NO: 276 | QSVLTQPPSA SGTPGQRVTI SCSGSLSNIG RNPVNWYQQL PGTAPKLLIY LDNLRLSGVP | 60 |
| Anti-CD73 | DRFSGSKSGT SASLASSGLQ SEDEADYYCA TWDDSHPGWT FGGGTKLTVL | 110 |
| variable light | | |
| chain | | |
| SEQ ID NO: 277 | SYAYS | 5 |
| Anti-CD73 CDR1 | | |
| heavy chain | | |
| SEQ ID NO: 278 | AISGSGGRTY YADSVKG | 17 |
| Anti-CD73 CDR2 | | |
| heavy chain | | |
| SEQ ID NO: 279 | LGYGRVDE | 8 |
| Anti-CD73 CDR3 | | |
| heavy chain | | |
| SEQ ID NO: 280 | SGSLSNIGRN PVN | 13 |
| Anti-CD73 CDR1 | | |
| light chain | | |
| SEQ ID NO: 281 | LDNLRLS | 7 |
| Anti-CD73 CDR2 | | |
| light chain | | |
| SEQ ID NO: 282 | ATWDDSEPGW T | 11 |
| Anti-CD73 CDR3 | | |
| light chain | | |

In an embodiment, an anti-CD73 scFv domain comprises a V_{H} domain and a V_{L} domain, wherein the V_{H} domain comprises the sequence shown in SEQ ID NO:275, or conservative amino acid substitutions thereof, and the light chain variable region (V_{L}) comprises the sequence shown in SEQ ID NO:276, or conservative amino acid substitutions thereof. In an embodiment, an anti-CD73 scFv domain comprises V_{H} and V_{L} regions that are each at least 99% identical to the sequences shown in SEQ ID NO:275 and SEQ ID NO:276, respectively. In an embodiment, an anti-CD73 scFv domain comprises V_{H} and V_{L} regions that are each at least 98% identical to the sequences shown in SEQ ID NO:275 and SEQ ID NO:276, respectively. In an embodiment, an anti-CD73 scFv domain comprises V_{H} and V_{L} regions that are each at least 97% identical to the sequences shown in SEQ ID NO:275 and SEQ ID NO:276, respectively. In an embodiment, an anti-CD73 scFv domain comprises V_{H} and V_{L} regions that are each at least 96% identical to the sequences shown in SEQ ID NO:275 and SEQ ID NO:276, respectively. In an embodiment, an anti-CD73 scFv domain comprises V_{H} and V_{L} regions that are each at least 95% identical to the sequences shown in SEQ ID NO:275 and SEQ ID NO:276, respectively. In an embodiment, an anti-CD73 scFv domain comprises V_{H} and V_{L} regions that are each at least 90% identical to the sequences shown in SEQ ID NO:275 and SEQ ID NO:276, respectively. In an embodiment, an anti-CD73 scFv domain comprises V_{H} and V_{L} regions that are each at least 85% identical to the sequences shown in SEQ ID NO:275 and SEQ ID NO:276, respectively. In an embodiment, an anti-CD73 scFv domain comprises V_{H} and V_{L} regions that are each at least 80% identical to the sequences shown in SEQ ID NO:275 and SEQ ID NO:276, respectively.

In an embodiment, an anti-CD73 scFv domain comprises the heavy chain CDR1, CDR2 and CDR3 domains having the sequences set forth in SEQ ID NO:277, SEQ ID NO:278, and/or SEQ ID NO:279, respectively, or conservative amino acid substitutions thereof, and/or light chain CDR1, CDR2 and CDR3 domains having the sequences set forth in SEQ ID NO:280, SEQ ID NO:281, and/or SEQ ID NO:282, respectively, or conservative amino acid substitutions thereof.

In an embodiment, the anti-CD73 binding domain includes a V_{H} and/or V_{L} sequence or a heavy chain and/or a light chain CDR1, CDR2, and/or CDR3 sequence as disclosed in U.S. Pat. No. 9,388,249; the disclosures of which are incorporated by reference herein. The amino acid sequences of additional exemplary CD73 binding scFv domains are provided in Table 40.

TABLE 40
Amino acid sequences of exemplary extracellular CD73 binding domains.

| Identifier | Sequence (One-Letter Amino Acid Symbols) | |
|---|---|---|
| SEQ ID NO: 283 | EVQLVESGGG LVQPGRSLRL SCAASGFTFD DYAMEWVRQA PGKGLEWVSG ISWNSGSIGY | 60 |
| Anti-CD73 | ADSVKGRFTI SRDNAKNSLY LQMNSLRAED TALYYCVRSG SYNYYYYGMD VWGQGTTVTV | 120 |
| variable heavy | SR | 122 |
| chain | | |
| SEQ ID NO: 284 | QSVLTQPPSA SGTPGQRVTI SCSGSSSNIG SNTVNWYQQL PGTAPKLLIY SNNQRPSGVP | 60 |
| Anti-CD73 | DRFSGSKSGT SASLAISGLQ SEDEADYYCA AWDDSLNGWV FGGGTKLTVL G | 111 |
| variable light | | |
| chain | | |
| SEQ ID NO: 285 | DYAMH | 5 |
| Anti-CD73 CDR1 | | |
| heavy chain | | |
| SEQ ID NO: 286 | GISWNSGSIG YADSVKG | 17 |
| Anti-CD73 CDR2 | | |
| heavy chain | | |
| SEQ ID NO: 287 | SGSYNYYYYG MDV | 13 |
| Anti-CD73 CDR3 | | |
| heavy chain | | |
| SEQ ID NO: 288 | SGSSSNIGSN TVN | 13 |
| Anti-CD73 CDR1 | | |
| light chain | | |
| SEQ ID NO: 289 | SNNQRPS | 7 |
| Anti-CD73 CDR2 | | |
| light chain | | |
| SEQ ID NO: 290 | AAWDDSLNG | 9 |
| Anti-CD73 CDR3 | | |
| light chain | | |

In an embodiment, an anti-CD73 scFv domain comprises a V_{H} domain and a V_{L} domain, wherein the V_{H} domain comprises the sequence shown in SEQ ID NO:283, or conservative amino acid substitutions thereof, and the light chain variable region (V_{L}) comprises the sequence shown in SEQ ID NO:284, or conservative amino acid substitutions thereof. In an embodiment, an anti-CD73 scFv domain comprises V_{H} and V_{L} regions that are each at least 99% identical to the sequences shown in SEQ ID NO:283 and SEQ ID NO:284, respectively. In an embodiment, an anti-CD73 scFv domain comprises V_{H} and V_{L} regions that are each at least 98% identical to the sequences shown in SEQ ID NO:283 and SEQ ID NO:284, respectively. In an embodiment, an anti-CD73 scFv domain comprises V_{H} and V_{L} regions that are each at least 97% identical to the sequences shown in SEQ ID NO:283 and SEQ ID NO:284, respectively. In an embodiment, an anti-CD73 scFv domain comprises V_{H} and V_{L} regions that are each at least 96% identical to the sequences shown in SEQ ID NO:283 and SEQ ID NO:284, respectively. In an embodiment, an anti-CD73 scFv domain comprises V_{H} and V_{L} regions that are each at least 95% identical to the sequences shown in SEQ ID NO:283 and SEQ ID NO:284, respectively. In an embodiment, an anti-CD73 scFv domain comprises V_{H} and V_{L} regions that are each at least 90% identical to the sequences shown in SEQ ID NO:283 and SEQ ID NO:284, respectively. In an embodiment, an anti-CD73 scFv domain comprises V_{H} and V_{L} regions that are each at least 85% identical to the sequences shown in SEQ ID NO:283 and SEQ ID NO:284, respectively. In an embodiment, an anti-CD73 scFv domain comprises V_{H} and V_{L} regions that are each at least 80% identical to the sequences shown in SEQ ID NO:283 and SEQ ID NO:284, respectively.

In an embodiment, an anti-CD73 scFv domain comprises the heavy chain CDR1, CDR2 and CDR3 domains having the sequences set forth in SEQ ID NO:277, SEQ ID NO:278, and/or SEQ ID NO:279, respectively, or conservative amino acid substitutions thereof, and/or light chain CDR1, CDR2 and CDR3 domains having the sequences set forth in SEQ ID NO:280, SEQ ID NO:281, and/or SEQ ID NO:282, respectively, or conservative amino acid substitutions thereof.

In an embodiment, the anti-CD73 binding domain includes a V_{H} and/or V_{L} sequence or a heavy chain and/or a light chain CDR1, CDR2, and/or CDR3 sequence, or conservative amino acid substitutions thereof, or nucleotides that encode such sequences, as disclosed in U.S. Pat. Nos. 10,822,426, the disclosures of which are incorporated by reference herein.

In an embodiment, the anti-CD73 binding domain includes a V_{H} and/or V_{L} sequence or a heavy chain and/or a light chain CDR1, CDR2, and/or CDR3 sequence, or conservative amino acid substitutions thereof, or nucleotides that encode such sequences, as disclosed in U.S. Patent Application Publication No. US 2019/0284293 A1, the disclosures of which are incorporated by reference herein.

In an embodiment, the anti-CD73 binding domain includes a V_{H} and/or V_{L} sequence or a heavy chain and/or a light chain CDR1, CDR2, and/or CDR3 sequence, or conservative amino acid substitutions thereof, or nucleotides that encode such sequences, as disclosed in U.S. Patent Application Publication No. US 2020/0392243 A1, the disclosures of which are incorporated by reference herein.

### 5. Extracellular TROP-2 Binding Domains

In an embodiment, a CCR comprises an extracellular domain, wherein the extracellular domain is a domain capable of binding to human TROP-2. In an embodiment, the extracellular domain binds to human TROP-2, also known as trophoblast cell-surface antigen-2, tumor-associated calcium signal transducer 2 or epithelial glycoprotein-1 antigen (EGP-1), which is encoded by TACSTD2. The function of TROP-2 and its role in tumor pathogenesis, including its activation of the ERK-MAPK pathway and PI3K-AKT pathway, are described in Cubas, et al., Mol. Cancer 2010, 9, 253; Gu, et al., Mol. Med Rep. 2018, 18, 1782-88; and McDougall, et al., Dev. Dyn. 2015, 244, 99-109, the disclosures of each of which are incorporated by reference herein. In an embodiment, the extracellular domain binds to murine or human TROP-2. In an embodiment, the extracellular TROP-2 binding domain is a scFv domain. In an embodiment, the TROP-2 scFv binding domain binds to murine TROP-2. In an embodiment, the TROP-2 scFv binding domain binds to human TROP-2. In an embodiment, a CCR of the present invention comprises a construct as shown in FIG. 34, wherein the V_{H} and V_{L} domains are anti-TROP-2 V_{H} and V_{L} domains, and the linker is as described herein.

In an embodiment, a CCR comprises an extracellular scFv domain that binds to TROP-2 and comprises V_{H}, V_{L}, or CDR domains, or nucleotides that encode such domains, described in U.S. Patent Application Publication No. US 2012/0237518 A1, the disclosures of which are incorporated by reference herein. The amino acid sequences of exemplary TROP-2 binding scFv domains are provided in Table 41.

TABLE 41
Amino acid sequences of exemplary TROP-2 binding scFv domains.

| Identifier | Sequence (One-Letter Amino Acid Symbols) | |
|---|---|---|
| SEQ ID NO: 291 | MEWSGVFIFL LSVTADVHSQ VQLQQSGPEL VRPGTSVRIS CKASGYTFTI YWLGWVKQRP | 60 |
| Anti-TROP-2 | GHGLEWIGNI FPGSAYINYN EKFKGKATLT ADTSSSTAYM QLSSLTSEDS AVYFCAREGS | 120 |
| variable heavy | NSGYWGQGTT LTVSS | 135 |
| chain | | |
| SEQ ID NO: 292 | QVQLVQSGAE VKKPGASVKV SCKASGYTFT IYWLGWVRQA PGQGLEWMGN IFPGSAYINY | 60 |
| Anti-TROP-2 | NEKFKGRVTI TADTSTSTAY MELSSLRSED TAVYYCAREG SNSGYWGQGT LVTVSS | 116 |
| variable heavy | | |
| chain | | |
| SEQ ID NO: 293 | QVQLVQSGPE VKKPGASVKV SCKASGYTFT IYWLGWVRQA PGQGLEWMGN IFPGSAYINY | 60 |
| Anti-TROP-2 | NEKFKGKVTI TADTSTSTAY MELSSLRSED TAVYFCAREG SNSGYWGQGT LVTVSS | 116 |
| variable heavy | | |
| chain | | |
| SEQ ID NO: 294 | QVQLVQSGPE VKKPGASVKV SCKASGYTFT IYWLGWVKQA PGQGLEWIGN IFPGSAYINY | 60 |
| Anti-TROP-2 | NEKFKGRVTI TADTSTSTAY MELSSLRSED TAVYFCAREG SNSGYWGQGT LVTVSS | 116 |
| variable heavy | | |
| chain | | |
| SEQ ID NO: 295 | QVQLVQSGAE VKKPGASVKV SCKASGYTFT IYWLGWVKQA PGQGLEWIGN IFPGSAYINY | 60 |
| Anti-TROP-2 | NEKFKGKATI TADTSTSTAY MELSSLRSED TAVYFCAREG SNSGYWGQGT LVTVSS | 116 |
| variable heavy | | |
| chain | | |
| SEQ ID NO: 296 | QVQLVQSGPE VVKPGASVKI SCKASGYTFT IYWLGWVKQA PGQGLEWIGN IFPGSAYINY | 60 |
| Anti-TROP-2 | NEKFKGKATL TADTSTSTAY MELSSLRSED TAVYFCAREG SNSGYWGQGT LLTVSS | 116 |
| variable heavy | | |
| chain | | |
| SEQ ID NO: 297 | MESQTQVLIS LLFWVSGTCG DIVMTQSPSS LSVSAGEKVT MTCKSSQSLL NSGNQQNYLA | 60 |
| Anti-TROP-2 | WYQQKPGQPP KLLIYGASTR ESGVPDRFTG SGSGTDFTLT INSVQAEDLA VYYCQSDHIY | 120 |
| variable light | PYTFGGGTKL EIK | 133 |
| chain | | |
| SEQ ID NO: 298 | DIVMTQSPDS LAVSLGERAT INCKSSQSLL NSGNQQNYLA WYQQKPGQPP KLLIYGASTR | 60 |
| Anti-TROP-2 | ESGVPDRFSG SGSGTDFTLT ISSLQAEDVA VYYCQSDHIY PYTFGQGTKL EIK | 113 |
| variable light | | |
| chain | | |
| SEQ ID NO: 299 | DIVMTQSPDS LAVSLGERVT INCKSSQSLL NSGNQQNYLA WYQQKPGQPP KLLIYGASTR | 60 |
| Anti-TROP-2 | ESGVPDRFSG SGSGTDFTLT ISSVQAEDVA VYYCQSDHIY PYTFGQGTKL EIK | 113 |
| variable light | | |
| chain | | |
| SEQ ID NO: 300 | DIVMTQSPDS LAVSAGERVT MNCKSSQSLL NSGNQQNYLA WYQQKPGQPP KLLIYGASTR | 60 |
| Anti-TROP-2 | ESGVPDRFSG SGSGTDFTLT ISSVQAEDVA VYYCQSDHIY PYTFGQGTKL EIK | 113 |
| variable light | | |
| chain | | |
| SEQ ID NO: 301 | IYWLG | 5 |
| Anti-TROP-2 CDR1 | | |
| heavy chain | | |
| SEQ ID NO: 302 | NIFPGSAYIN YNEKFK | 16 |
| Anti-TROP-2 CDR2 | | |
| heavy chain | | |
| SEQ ID NO: 303 | EGSNSGY | 7 |
| Anti-TROP-2 CDR3 | | |
| heavy chain | | |
| SEQ ID NO: 304 | KSSQSLLNSG NQQNYL | 16 |
| Anti-TROP-2 CDR1 | | |
| light chain | | |
| SEQ ID NO: 305 | GASTRES | 7 |
| Anti-TROP-2 CDR2 | | |
| light chain | | |
| SEQ ID NO: 306 | QSDHIYPYT | 9 |
| Anti-TROP-2 CDR3 | | |
| light chain | | |

In an embodiment, an anti-TROP-2 scFv domain comprises a V_{H} domain and a V_{L} domain, wherein the V_{H} domain comprises a sequence selected from the group consisting of SEQ ID NO:291, SEQ ID NO:292, SEQ ID NO:293, SEQ ID NO:294, SEQ ID NO:295, SEQ ID NO:296, and conservative amino acid substitutions thereof, and the light chain variable region (V_{L}) comprises a sequence selected from the group consisting of SEQ ID NO:297, SEQ ID NO:298, SEQ ID NO:299, SEQ ID NO:300, and conservative amino acid substitutions thereof. In an embodiment, an anti-TROP-2 scFv domain comprises a V_{H} region that is 99% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 99% identical to a sequence selected from the group consisting of SEQ ID NO:297, SEQ ID NO:298, SEQ ID NO:299, and SEQ ID NO:300. In an embodiment, an anti-TROP-2 scFv domain comprises a V_{H} region that is 98% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 98% identical to a sequence selected from the group consisting of SEQ ID NO:297, SEQ ID NO:298, SEQ ID NO:299, and SEQ ID NO:300. In an embodiment, an anti-TROP-2 scFv domain comprises a V_{H} region that is 97% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 97% identical to a sequence selected from the group consisting of SEQ ID NO:297, SEQ ID NO:298, SEQ ID NO:299, and SEQ ID NO:300. In an embodiment, an anti-TROP-2 scFv domain comprises a V_{H} region that is 96% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 96% identical to a sequence selected from the group consisting of SEQ ID NO:297, SEQ ID NO:298, SEQ ID NO:299, and SEQ ID NO:300. In an embodiment, an anti-TROP-2 scFv domain comprises a V_{H} region that is 95% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 95% identical to a sequence selected from the group consisting of SEQ ID NO:297, SEQ ID NO:298, SEQ ID NO:299, and SEQ ID NO:300. In an embodiment, an anti-TROP-2 scFv domain comprises a V_{H} region that is 90% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 90% identical to a sequence selected from the group consisting of SEQ ID NO:297, SEQ ID NO:298, SEQ ID NO:299, and SEQ ID NO:300. In an embodiment, an anti-TROP-2 scFv domain comprises a V_{H} region that is 85% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 85% identical to a sequence selected from the group consisting of SEQ ID NO:297, SEQ ID NO:298, SEQ ID NO:299, and SEQ ID NO:300. In an embodiment, an anti-TROP-2 scFv domain comprises a V_{H} region that is 80% identical to a sequence selected from the group consisting of SEQ ID NO:291, SEQ ID NO:292, SEQ ID NO:293, SEQ ID NO:294, SEQ ID NO:295, and SEQ ID NO:296, and a V_{L} region that is at least 80% identical to a sequence selected from the group consisting of SEQ ID NO:297, SEQ ID NO:298, SEQ ID NO:299, and SEQ ID NO:300.

In an embodiment, an anti-TROP-2 scFv domain comprises the heavy chain CDR1, CDR2 and CDR3 domains having the sequences set forth in SEQ ID NO:301, SEQ ID NO:302, and/or SEQ ID NO:303, respectively, or conservative amino acid substitutions thereof, and/or light chain CDR1, CDR2 and CDR3 domains having the sequences set forth in SEQ ID NO:304, SEQ ID NO:305, and/or SEQ ID NO:306, respectively, or conservative amino acid substitutions thereof.

In an embodiment, a CCR comprises an extracellular scFv domain that binds to TROP-2 and comprises a V_{H} chain according to SEQ ID NO:292. In an embodiment, a CCR comprises an extracellular scFv domain that comprises a V_{H} chain according to SEQ ID NO:298 wherein at least one amino acid modification substituting Ala at position 9 with Pro, Lys at position 12 with Val, Val at position 20 with Ile, Arg at position 38 with Lys, Met at position 48 with Ile, Arg at position 67 with Lys, Val at position 68 with Ala, Ile at position 70 with Leu, Tyr at position 95 with Phe, or Val at position 112 with Leu is introduced in the amino acid sequence of SEQ ID NO:292. In an embodiment, the extracellular domain is a scFv domain. In an embodiment, a CCR comprises an extracellular scFv domain that binds to TROP-2 and comprises a V_{L} chain according to SEQ ID NO:298.

In an embodiment, the anti-TROP-2 binding domain includes a V_{H} and/or V_{L} sequence or a heavy chain and/or a light chain CDR1, CDR2, and/or CDR3 sequence as disclosed in U.S. Pat. No. 9,399,074, the disclosures of which are incorporated by reference herein. In an embodiment, the anti-TROP-2 binding domain includes a V_{H} and/or V_{L} sequence or a heavy chain and/or a light chain CDR1, CDR2, and/or CDR3 sequence, or a nucleotide encoding such a sequence, for the antibodies m7E6, h7E6, h7E6_SVG, h7E6_SVGL, m6G11, h6G11, or h6G11-FKG_SF, as disclosed in U.S. Pat. No. 9,399,074, the disclosures of which are incorporated by reference herein. In an embodiment, the anti-TROP-2 binding domain includes a V_{H} and/or V_{L} sequence or a heavy chain and/or a light chain CDR1, CDR2, and/or CDR3 sequence, or a nucleotide encoding such a sequence, for the antibodies m7E6, h7E6_SVG, h7E6_SVG4, h7E6_SVG19, h7E6_SVG6, h7E6_SVG20, h7E6_SVG22, h7E6_SVG28, h7E6_SVG30, h7E6_SVGL, h7E6_SVGL1, h7E6_SVGL2, h7E6_SVGL3, h7E6_SVGL4, h7E6_SVGL5, h7E6_SVGN, m6G11, h6G11, or h6G11_FKG_SF, as disclosed in U.S. Pat. No. 9,399,074, the disclosures of which are incorporated by reference herein. The amino acid sequences of exemplary TROP-2 binding scFv domains are provided in Table 42.

TABLE 42
Amino acid sequences of exemplary TROP-2 binding scFv domains.

| Identifier | Sequence (One-Letter Amino Acid Symbols) | |
|---|---|---|
| SEQ ID NO: 307 | QVQLKESGPG LVAPSQSLSI TCTVSGFSLT SYGVEWVRQP PGKGLEWLGV IWTGGSTDYN | 60 |
| Anti-TROP-2 m7E6 | SALMSRLSIN KDNSKSQVFL KMNSLQTDDT AMYYCARDGD YDRYTMDYWG QGTSVTVSS | 119 |
| variable heavy | | |
| chain | | |
| SEQ ID NO: 308 | DIVLTQSPAS LAVSLGQRAT ISCRASKSVS TSGYSYMHWY QQKPGQPPKL LIYLASNLES | 60 |
| Anti-TROP-2 m7E6 | GVPARFSGSG SGTDFTLNIH PVEEEDAATY YCQHSRELPY TFGGGTKLEI K | 111 |
| variable light | | |
| chain | | |
| SEQ ID NO: 309 | QVQLCESGPG LVKPSETLSL TCTVSGGSIS SYGVEWIRQP PGKGLEWIGV IWTGGSTDYN | 60 |
| Anti-TROP-2 h7E6 | SALMSRVTIS VDTSKNQFSL KLSSVTAADT AVYYCARDGD YDRYTMDYWG QGTLVTVSS | 119 |
| variable heavy | | |
| chain | | |
| SEQ ID NO: 310 | DIVMTQSPDS LAVSLGERAT INCRASKSVS TSGYSYMHWY QQKPGQPPKL LIYLASNLES | 60 |
| Anti-TROP-2 h7E6 | GVPDRFSGSG SGTDFTLTIS SLQAEDVAVY YCQHSRELPY TFGQGTKLEI K | 111 |
| variable light | | |
| chain | | |
| SEQ ID NO: 311 | QVQLQESGPG LVKPSETLSL TCTVSGGSIS SYGVEWIRQP PGKGLEWIGV IWTSGVTDYN | 60 |
| Anti-TROP-2 | SALMGRVTIS VDTSKNQFSL KLSSVTAADT AVYYCARDGD YDRYTMDYWG QGTLVTVSS | 119 |
| h7E6_SVGL | | |
| variable heavy | | |
| chain | | |
| SEQ ID NO: 312 | DIVMTQSPDS LAVSLGERAT INCRASKSVS TSLYSYMHWY QQKPGQPPKL LIYLASNLES | 60 |
| Anti-TROP-2 | GVPDRFSGSG SGTDFTLTIS SLQAEDVAVY YCQHSRELPY TFGQGTKLEI K | 111 |
| h7E6_SVGL | | |
| variable light | | |
| chain | | |
| SEQ ID NO: 313 | QVQLQQPGAE LVRPGASVKL SCKASGYTFT SYWINWVKQR PGHGLEWIGN IYPSDSYSNY | 60 |
| Anti-TROP-2 | NQKFKDKATL TVDKSSSTAY MQVSSPTSED SAVYYCTYGS SFDYWGQGTT VTVSS | 115 |
| m6G11 variable | | |
| heavy chain | | |
| SEQ ID NO: 314 | DILLTQSPAI LSVSPGERVS ESCRASQTIG TSIHWYQQRT NGSPRLLIKY ASESISGIPS | 60 |
| Anti-TROP-2 | RFSGSGSGTD FTLSINSVES EDIADYYCQQ SNSWPFTFGS GTKLEIK | 107 |
| m6G11 variable | | |
| light chain | | |
| SEQ ID NO: 315 | QVQLVQSGAE VKKPGASVKV SCKASGYTFT SYWINWVRQA PGQGLEWMGN IYPSDSYSNY | 60 |
| Anti-TROP-2 | NQKFKDRVTM TRDTSTSTVY MELSSLRSED TAVYYCARGS SFDYWGQGTL VTVSS | 115 |
| h6G11 variable | | |
| heavy chain | | |
| SEQ ID NO: 316 | EIVLTQSPAT LSLSPGERAT LSCRASQTIG TSIHWYQQKP GQAPRLLIYY ASESISGIPA | 60 |
| Anti-TROP-2 | RFSGSGSGTD FTLTISSLEP EDFAVYYCQQ SNSWPFTFGQ GTKLEIK | 107 |
| h6G11 variable | | |
| light chain | | |
| SEQ ID NO: 317 | QVQLVQSGAE VKKPGASVKV SCKASGYTFT SYWINWVRQA PGQGLEWMGN IFPSDSYSNY | 60 |
| Anti-TROP-2 | NKKFKDRVTM TRDTSTSTVY MELSSLRSED TAVYYCARGS GFDYWGQGTL VTVSS | 115 |
| h6G11-FKG SF | | |
| variable heavy | | |
| chain | | |
| SEQ ID NO: 318 | EIVLTQSPAT LSLSPGERAT LSCRASQTIG TSIHWYQQKP GQAPRLLIYY ASESISGIPA | 60 |
| Anti-TROP-2 | RFSGSGSGTD FTLTISSLEP EDFAVYYCSQ SFSWPFTFGQ GTKLEIK | 107 |
| h6G11-FKG SF | | |
| variable light | | |
| chain | | |
| SEQ ID NO: 319 | SYGVH | 5 |
| Anti-TROP-2 CDR1 | | |
| heavy chain | | |
| SEQ ID NO: 320 | WTSGV | 5 |
| Anti-TROP-2 CDR2 | | |
| heavy chain | | |
| SEQ ID NO: 321 | DGDYDRYTMD Y | 11 |
| Anti-TROP-2 CDR3 | | |
| heavy chain | | |
| SEQ ID NO: 322 | RASKSVSTSG YSYMH | 15 |
| Anti-TROP-2 CDR1 | | |
| light chain | | |
| SEQ ID NO: 323 | LASNLES | 7 |
| Anti-TROP-2 CDR2 | | |
| light chain | | |
| SEQ ID NO: 324 | QHSRELPYT | 9 |
| Anti-TROP-2 CDR3 | | |
| light chain | | |

In an embodiment, an anti-TROP-2 scFv domain comprises a heavy chain variable region (V_{H}) domain and/or a light chain variable region (V_{L}) domain of the scFv antibody m7E6, or conservative amino acid substitutions thereof. In an embodiment, an anti-TROP-2 scFv domain comprises a V_{H} domain and/or a V_{L} domain, wherein the V_{H} domain comprises the sequence shown in SEQ ID NO:307, or conservative amino acid substitutions thereof, and the light chain variable region (V_{L}) comprises the sequence shown in SEQ ID NO:308, or conservative amino acid substitutions thereof. In an embodiment, an anti-TROP-2 scFv domain comprises V_{H} and/or V_{L} regions that are each at least 99% identical to the sequences shown in SEQ ID NO:307 and SEQ ID NO:308, respectively. In an embodiment, an anti-TROP-2 scFv domain comprises V_{H} and/or V_{L} regions that are each at least 98% identical to the sequences shown in SEQ ID NO:307 and SEQ ID NO:308, respectively. In an embodiment, an anti-TROP-2 scFv domain comprises V_{H} and/or V_{L} regions that are each at least 97% identical to the sequences shown in SEQ ID NO:307 and SEQ ID NO:308, respectively. In an embodiment, an anti-TROP-2 scFv domain comprises V_{H} and/or V_{L} regions that are each at least 96% identical to the sequences shown in SEQ ID NO:307 and SEQ ID NO:308, respectively. In an embodiment, an anti-TROP-2 scFv domain comprises V_{H} and/or V_{L} regions that are each at least 95% identical to the sequences shown in SEQ ID NO:307 and SEQ ID NO:308, respectively. In an embodiment, an anti-TROP-2 scFv domain comprises V_{H} and/or V_{L} regions that are each at least 90% identical to the sequences shown in SEQ ID NO:307 and SEQ ID NO:308, respectively. In an embodiment, an anti-TROP-2 scFv domain comprises V_{H} and/or V_{L} regions that are each at least 85% identical to the sequences shown in SEQ ID NO:307 and SEQ ID NO:308, respectively. In an embodiment, an anti-TROP-2 scFv domain comprises V_{H} and/or V_{L} regions that are each at least 80% identical to the sequences shown in SEQ ID NO:307 and SEQ ID NO:308, respectively.

In an embodiment, an anti-TROP-2 scFv domain comprises a heavy chain variable region (V_{H}) domain and/or a light chain variable region (V_{L}) domain of the scFv antibody h7E6, or conservative amino acid substitutions thereof. In an embodiment, an anti-TROP-2 scFv domain comprises a V_{H} domain and/or a V_{L} domain, wherein the V_{H} domain comprises the sequence shown in SEQ ID NO:309, or conservative amino acid substitutions thereof, and the light chain variable region (V_{L}) comprises the sequence shown in SEQ ID NO:310, or conservative amino acid substitutions thereof. In an embodiment, an anti-TROP-2 scFv domain comprises V_{H} and/or V_{L} regions that are each at least 99% identical to the sequences shown in SEQ ID NO:309 and SEQ ID NO:310, respectively. In an embodiment, an anti-TROP-2 scFv domain comprises V_{H} and/or V_{L} regions that are each at least 98% identical to the sequences shown in SEQ ID NO:309 and SEQ ID NO:310, respectively. In an embodiment, an anti-TROP-2 scFv domain comprises V_{H} and/or V_{L} regions that are each at least 97% identical to the sequences shown in SEQ ID NO:309 and SEQ ID NO:310, respectively. In an embodiment, an anti-TROP-2 scFv domain comprises V_{H} and/or V_{L} regions that are each at least 96% identical to the sequences shown in SEQ ID NO:309 and SEQ ID NO:310, respectively. In an embodiment, an anti-TROP-2 scFv domain comprises V_{H} and/or V_{L} regions that are each at least 95% identical to the sequences shown in SEQ ID NO:309 and SEQ ID NO:310, respectively. In an embodiment, an anti-TROP-2 scFv domain comprises V_{H} and/or V_{L} regions that are each at least 90% identical to the sequences shown in SEQ ID NO:309 and SEQ ID NO:310, respectively. In an embodiment, an anti-TROP-2 scFv domain comprises V_{H} and/or V_{L} regions that are each at least 85% identical to the sequences shown in SEQ ID NO:309 and SEQ ID NO:310, respectively. In an embodiment, an anti-TROP-2 scFv domain comprises V_{H} and/or V_{L} regions that are each at least 80% identical to the sequences shown in SEQ ID NO:309 and SEQ ID NO:310, respectively.

In an embodiment, an anti-TROP-2 scFv domain comprises a heavy chain variable region (V_{H}) domain and/or a light chain variable region (V_{L}) domain of the scFv antibody h7E6_SVG, or conservative amino acid substitutions thereof. In an embodiment, an anti-TROP-2 scFv domain comprises a V_{H} domain and/or a V_{L} domain, wherein the V_{H} domain comprises the sequence shown in SEQ ID NO:311, or conservative amino acid substitutions thereof, and the light chain variable region (V_{L}) comprises the sequence shown in SEQ ID NO:310, or conservative amino acid substitutions thereof. In an embodiment, an anti-TROP-2 scFv domain comprises V_{H} and/or V_{L} regions that are each at least 99% identical to the sequences shown in SEQ ID NO:311 and SEQ ID NO:310, respectively. In an embodiment, an anti-TROP-2 scFv domain comprises V_{H} and/or V_{L} regions that are each at least 98% identical to the sequences shown in SEQ ID NO:311 and SEQ ID NO:310, respectively. In an embodiment, an anti-TROP-2 scFv domain comprises V_{H} and/or V_{L} regions that are each at least 97% identical to the sequences shown in SEQ ID NO:311 and SEQ ID NO:310, respectively. In an embodiment, an anti-TROP-2 scFv domain comprises V_{H} and/or V_{L} regions that are each at least 96% identical to the sequences shown in SEQ ID NO:311 and SEQ ID NO:310, respectively. In an embodiment, an anti-TROP-2 scFv domain comprises V_{H} and/or V_{L} regions that are each at least 95% identical to the sequences shown in SEQ ID NO:311 and SEQ ID NO:310, respectively. In an embodiment, an anti-TROP-2 scFv domain comprises V_{H} and/or V_{L} regions that are each at least 90% identical to the sequences shown in SEQ ID NO:311 and SEQ ID NO:310, respectively. In an embodiment, an anti-TROP-2 scFv domain comprises V_{H} and/or V_{L} regions that are each at least 85% identical to the sequences shown in SEQ ID NO:311 and SEQ ID NO:310, respectively. In an embodiment, an anti-TROP-2 scFv domain comprises V_{H} and/or V_{L} regions that are each at least 80% identical to the sequences shown in SEQ ID NO:311 and SEQ ID NO:310, respectively.

In an embodiment, an anti-TROP-2 scFv domain comprises a heavy chain variable region (V_{H}) domain and/or a light chain variable region (V_{L}) domain of the scFv antibody h7E6_SVGL, or conservative amino acid substitutions thereof. In an embodiment, an anti-TROP-2 scFv domain comprises a V_{H} domain and/or a V_{L} domain, wherein the V_{H} domain comprises the sequence shown in SEQ ID NO:311, or conservative amino acid substitutions thereof, and the light chain variable region (V_{L}) comprises the sequence shown in SEQ ID NO:312, or conservative amino acid substitutions thereof. In an embodiment, an anti-TROP-2 scFv domain comprises V_{H} and/or V_{L} regions that are each at least 99% identical to the sequences shown in SEQ ID NO:311 and SEQ ID NO:312, respectively. In an embodiment, an anti-TROP-2 scFv domain comprises V_{H} and/or V_{L} regions that are each at least 98% identical to the sequences shown in SEQ ID NO:311 and SEQ ID NO:312, respectively. In an embodiment, an anti-TROP-2 scFv domain comprises V_{H} and/or V_{L} regions that are each at least 97% identical to the sequences shown in SEQ ID NO:311 and SEQ ID NO:312, respectively. In an embodiment, an anti-TROP-2 scFv domain comprises V_{H} and/or V_{L} regions that are each at least 96% identical to the sequences shown in SEQ ID NO:311 and SEQ ID NO:312, respectively. In an embodiment, an anti-TROP-2 scFv domain comprises V_{H} and/or V_{L} regions that are each at least 95% identical to the sequences shown in SEQ ID NO:311 and SEQ ID NO:312, respectively. In an embodiment, an anti-TROP-2 scFv domain comprises V_{H} and/or V_{L} regions that are each at least 90% identical to the sequences shown in SEQ ID NO:311 and SEQ ID NO:312, respectively. In an embodiment, an anti-TROP-2 scFv domain comprises V_{H} and/or V_{L} regions that are each at least 85% identical to the sequences shown in SEQ ID NO:311 and SEQ ID NO:312, respectively. In an embodiment, an anti-TROP-2 scFv domain comprises V_{H} and/or V_{L} regions that are each at least 80% identical to the sequences shown in SEQ ID NO:311 and SEQ ID NO:312, respectively.

In an embodiment, an anti-TROP-2 scFv domain comprises a heavy chain variable region (V_{H}) domain and/or a light chain variable region (V_{L}) domain of the scFv antibody m6G11, or conservative amino acid substitutions thereof. In an embodiment, an anti-TROP-2 scFv domain comprises a V_{H} domain and/or a V_{L} domain, wherein the V_{H} domain comprises the sequence shown in SEQ ID NO:313, or conservative amino acid substitutions thereof, and the light chain variable region (V_{L}) comprises the sequence shown in SEQ ID NO:314, or conservative amino acid substitutions thereof. In an embodiment, an anti-TROP-2 scFv domain comprises V_{H} and/or V_{L} regions that are each at least 99% identical to the sequences shown in SEQ ID NO:313 and SEQ ID NO:314, respectively. In an embodiment, an anti-TROP-2 scFv domain comprises V_{H} and/or V_{L} regions that are each at least 98% identical to the sequences shown in SEQ ID NO:313 and SEQ ID NO:314, respectively. In an embodiment, an anti-TROP-2 scFv domain comprises V_{H} and/or V_{L} regions that are each at least 97% identical to the sequences shown in SEQ ID NO:313 and SEQ ID NO:314, respectively. In an embodiment, an anti-TROP-2 scFv domain comprises V_{H} and/or V_{L} regions that are each at least 96% identical to the sequences shown in SEQ ID NO:313 and SEQ ID NO:314, respectively. In an embodiment, an anti-TROP-2 scFv domain comprises V_{H} and/or V_{L} regions that are each at least 95% identical to the sequences shown in SEQ ID NO:313 and SEQ ID NO:314, respectively. In an embodiment, an anti-TROP-2 scFv domain comprises V_{H} and/or V_{L} regions that are each at least 90% identical to the sequences shown in SEQ ID NO:313 and SEQ ID NO:314, respectively. In an embodiment, an anti-TROP-2 scFv domain comprises V_{H} and/or V_{L} regions that are each at least 85% identical to the sequences shown in SEQ ID NO:313 and SEQ ID NO:314, respectively. In an embodiment, an anti-TROP-2 scFv domain comprises V_{H} and/or V_{L} regions that are each at least 80% identical to the sequences shown in SEQ ID NO:313 and SEQ ID NO:314, respectively.

In an embodiment, an anti-TROP-2 scFv domain comprises a heavy chain variable region (V_{H}) domain and/or a light chain variable region (V_{L}) domain of the scFv antibody h6G11, or conservative amino acid substitutions thereof. In an embodiment, an anti-TROP-2 scFv domain comprises a V_{H} domain and/or a V_{L} domain, wherein the V_{H} domain comprises the sequence shown in SEQ ID NO:315, or conservative amino acid substitutions thereof, and the light chain variable region (V_{L}) comprises the sequence shown in SEQ ID NO:316, or conservative amino acid substitutions thereof. In an embodiment, an anti-TROP-2 scFv domain comprises V_{H} and/or V_{L} regions that are each at least 99% identical to the sequences shown in SEQ ID NO:315 and SEQ ID NO:316, respectively. In an embodiment, an anti-TROP-2 scFv domain comprises V_{H} and/or V_{L} regions that are each at least 98% identical to the sequences shown in SEQ ID NO:315 and SEQ ID NO:316, respectively. In an embodiment, an anti-TROP-2 scFv domain comprises V_{H} and/or V_{L} regions that are each at least 97% identical to the sequences shown in SEQ ID NO:315 and SEQ ID NO:316, respectively. In an embodiment, an anti-TROP-2 scFv domain comprises V_{H} and/or V_{L} regions that are each at least 96% identical to the sequences shown in SEQ ID NO:315 and SEQ ID NO:316, respectively. In an embodiment, an anti-TROP-2 scFv domain comprises V_{H} and/or V_{L} regions that are each at least 95% identical to the sequences shown in SEQ ID NO:315 and SEQ ID NO:316, respectively. In an embodiment, an anti-TROP-2 scFv domain comprises V_{H} and/or V_{L} regions that are each at least 90% identical to the sequences shown in SEQ ID NO:315 and SEQ ID NO:316, respectively. In an embodiment, an anti-TROP-2 scFv domain comprises V_{H} and/or V_{L} regions that are each at least 85% identical to the sequences shown in SEQ ID NO:315 and SEQ ID NO:316, respectively. In an embodiment, an anti-TROP-2 scFv domain comprises V_{H} and/or V_{L} regions that are each at least 80% identical to the sequences shown in SEQ ID NO:315 and SEQ ID NO:316, respectively.

In an embodiment, an anti-TROP-2 scFv domain comprises a heavy chain variable region (V_{H}) domain and/or a light chain variable region (V_{L}) domain of the scFv antibody h6G11-FKG_SF, or conservative amino acid substitutions thereof. In an embodiment, an anti-TROP-2 scFv domain comprises a V_{H} domain and/or a V_{L} domain, wherein the V_{H} domain comprises the sequence shown in SEQ ID NO:317, or conservative amino acid substitutions thereof, and the light chain variable region (V_{L}) comprises the sequence shown in SEQ ID NO:318, or conservative amino acid substitutions thereof. In an embodiment, an anti-TROP-2 scFv domain comprises V_{H} and/or V_{L} regions that are each at least 99% identical to the sequences shown in SEQ ID NO:317 and SEQ ID NO:318, respectively. In an embodiment, an anti-TROP-2 scFv domain comprises V_{H} and/or V_{L} regions that are each at least 98% identical to the sequences shown in SEQ ID NO:317 and SEQ ID NO:318, respectively. In an embodiment, an anti-TROP-2 scFv domain comprises V_{H} and/or V_{L} regions that are each at least 97% identical to the sequences shown in SEQ ID NO:317 and SEQ ID NO:318, respectively. In an embodiment, an anti-TROP-2 scFv domain comprises V_{H} and/or V_{L} regions that are each at least 96% identical to the sequences shown in SEQ ID NO:317 and SEQ ID NO:318, respectively. In an embodiment, an anti-TROP-2 scFv domain comprises V_{H} and/or V_{L} regions that are each at least 95% identical to the sequences shown in SEQ ID NO:317 and SEQ ID NO:318, respectively. In an embodiment, an anti-TROP-2 scFv domain comprises V_{H} and/or V_{L} regions that are each at least 90% identical to the sequences shown in SEQ ID NO:317 and SEQ ID NO:318, respectively. In an embodiment, an anti-TROP-2 scFv domain comprises V_{H} and/or V_{L} regions that are each at least 85% identical to the sequences shown in SEQ ID NO:317 and SEQ ID NO:318, respectively. In an embodiment, an anti-TROP-2 scFv domain comprises V_{H} and/or V_{L} regions that are each at least 80% identical to the sequences shown in SEQ ID NO:317 and SEQ ID NO:318, respectively.

In an embodiment, an anti-TROP-2 scFv domain comprises the heavy chain CDR1, CDR2 and CDR3 domains having the sequences set forth in SEQ ID NO:319, SEQ ID NO:320, and/or SEQ ID NO:321, respectively, or conservative amino acid substitutions thereof, and/or light chain CDR1, CDR2 and CDR3 domains having the sequences set forth in SEQ ID NO:322, SEQ ID NO:323, and/or SEQ ID NO:324, respectively, or conservative amino acid substitutions thereof.

The nucleotide sequences encoding exemplary TROP-2 binding V_{H} and V_{L} domains for scFv domains for m7E6, h7E6, h7E6_SVG, h7E6_SVGL, m6G11, h6G11, and h6G11-FKG_SF are provided in Table 43 and are further described in U.S. Pat. No. 9,399,074, the disclosures of which are incorporated by reference herein. In an embodiment, a nucleotide sequence in Table 43 is codon-optimized to improve protein expression.

TABLE 43
Nucleotide sequences of exemplary TROP-2 binding scFv domains.

| Identifier | Sequence (One-Letter Nucleotide Symbols) | |
|---|---|---|
| SEQ ID NO: 325 | CAGGTCCAAC TGCAGGAATC AGGTCCAGGC CTGGTGAAAC CGTCTGAAAC CCTGAGCCTG | 60 |
| Anti-TROP-2 m7E6 | ACATGCACCG TGAGCGGTGG TAGTATTAGC TCTTACGGCG TCCATTGGAT CCGTCAACCG | 120 |
| variable heavy | CCTGGTAAAG GTCTGGAATG GATTGGCGTG ATCTGGACCG GTGGTAGCAC CGACTATAAC | 180 |
| chain | AGCGCACTGA TGAGCCGCGT GACCATCTCG GTAGACACGT CGAAAAACCA GTTCAGCCTG | 240 |
| | AAACTGAGCA GCGTGACCGC CGCGGATACC GCTGTTTATT ACTGCGCACG CGACGGGGAT | 300 |
| | TATGATCGCT ACACCATGGA TTATTGGGGC CAGGGTACCC TGGTCACCGT CTCCTCA | 357 |
| SEQ ID NO: 326 | GACATTGTGC TGACACAGTC TCCTGCTTCC TTAGCTGTAT CTCIGGGGCA GAGGGCCACC | 60 |
| Anti-TROP-2 m7E6 | ATCTCATGCA GGGCCAGCAA AAGTGTCAGT ACATCTGGCT ATAGTTATAT GCACTGGTAC | 120 |
| variable light | CAACAGAAAC CAGGACAGCC ACCCAAACTC CTCATCTATC TTGCATCCAA CCTAGAATCT | 180 |
| chain | GGGGTCCCTG CCAGGTTCAG TGGCAGTGGG TCTGGGACAG ACTTCACCCT CAACATCCAT | 240 |
| | CCTGTGGAGG AGGAGGATGC TGCAACCTAT TACTGTCAGC ACAGTAGGGA GCTTCCGTAC | 300 |
| | ACGTTCGGAG GGGGGACCAA GCTGGAGATC AAA | 333 |
| SEQ ID NO: 327 | CAGGTCCAAC TGCAGGAATC AGGTCCAGGC CTGGTGAAAC CGTCTGAAAC CCTGAGCCTG | 60 |
| Anti-TROP-2 h7E6 | ACATGCACCG TGAGCGGTGG TAGTATTAGC TCTTACGGCG TCCATTGGAT CCGTCAACCG | 120 |
| variable heavy | CCTGGTAAAG GTCTGGAATG GATTGGCGTG ATCTGGACCG GTGGTAGCAC CGACTATAAC | 180 |
| chain | AGCGCACTGA TGAGCCGCGT GACCATCTCG GTAGACACGT CGAAAAACCA GTTCAGCCTG | 240 |
| | AAACTGAGCA GCGTGACCGC CGCGGATACC GCTGTTTATT ACTGCGCACG CGACGGGGAT | 300 |
| | TATGATCGCT ACACCATGGA TTATTGGGGC CAGGGTACCC TGGTCACCGT CTCCTCA | 357 |
| SEQ ID NO: 328 | GATATCGTAA TGACCCAATC TCCGGATTCG CTGGCGGTAT CACTGGGCGA ACGTGCCACG | 60 |
| Anti-TROP-2 h7E6 | ATTAACTGCC GTGCAAGCAA ATCAGTGTCG ACCTCCGGCT ACAGCTATAT GCACTGGTAT | 120 |
| variable light | CAACAGAAAC CGGGCCAGCC GCCGAAACTG CTGATCTATC TGGCTAGCAA CCTGGAGAGC | 180 |
| chain | GGTGTGCCTG ATCGCTTTAG TGGCTCCGGT AGCGGTACCG ATTTCACGCT GACCATCAGC | 240 |
| | TCCCTGCAGG CAGAAGACGT GGCCGTGTAT TATTGTCAGC ACAGCCGTGA GCTGCCGTAT | 300 |
| | ACTTTTGGCC AGGGGACAAA ACTGGAAATC AAA | 333 |
| SEQ ID NO: 329 | CAGGTCCAAC TGCAGGAATC AGGTCCAGGC CTGGTGAAAC CGTCTGAAAC CCTGAGCCTG | 60 |
| Anti-TROP-2 | ACATGCACCG TGAGCGGTGG TAGTATTAGC TCTTACGGCG TCCATTGGAT CCGTCAACCG | 120 |
| h7E6_SVGL | CCTGGTAAAG GTCTGGAATG GATTGGCGTG ATCTGGACCA GTGGTGTGAC CGACTATAAC | 180 |
| variable heavy | AGCGCACTGA TGGGCCGCGT GACCATCTCG GTAGACACGT CGAAAAACCA GTTCAGCCTG | 240 |
| chain | AAACTGAGCA GCGTGACCGC CGCGGATACC GCTGTTTATT ACTGCGCACG CGACGGGGAT | 300 |
| | TATGATCGCT ACACCATGGA TTATTGGGGC CAGGGTACCC TGGTCACCGT CTCCTCA | 357 |
| SEQ ID NO: 330 | GATATCGTAA TGACCCAATC TCCGGATTCG CTGGCGGTAT CACTGGGCGA ACGTGCCACG | 60 |
| Anti-TROP-2 | ATTAACTGCC GTGCAAGCAA ATCAGTGTCG ACCTCCTTGT ACAGCTATAT GCACTGGTAT | 120 |
| h7E6_SVGL | CAACAGAAAC CGGGCCAGCC GCCGAAACTG CTGATCTATC TGGCTAGCAA CCTGGAGAGC | 180 |
| variable light | GGTGTGCCTG ATCGCTTTAG TGGCTCCGGT AGCGGTACCG ATTTCACGCT GACCATCAGC | 240 |
| chain | TCCCTGCAGG CAGAAGACGT GGCCGTGTAT TATTGTCAGC ACAGCCGTGA GCTGCCGTAT | 300 |
| | ACTTTTGGCC AGGGGACAAA ACTGGAAATC AAA | 333 |
| SEQ ID NO: 331 | CAGGTCCAAC TGCAGCAGCC TGGGGCTGAG CTGGTGAGGC CTGGGGCTTC AGTGAAGCTG | 60 |
| Anti-TROP-2 | TCCTGCAAGG CTTCTGGCTA CACCTTCACC AGCTACTGGA TAAACTGGGT GAAGCAGAGG | 120 |
| m6G11 variable | CCTGGACATG GCCTTGAGTG GATCGGAAAT ATTTATCCTT CTGATAGTTA TTCTAACTAC | 180 |
| heavy chain | AATCAAAAGT TCAAGGACAA GGCCACATTG ACTGTAGACA AATCCTCCAG CACAGCCTAC | 240 |
| | ATGCAGGTCA GCAGCCCGAC ATCTGAGGAC TCTGCGGTCT ATTACTGTAC GTACGGTAGT | 300 |
| | AGCTTTGACT ACTGGGGCCA AGGCACCACG GTCACCGTCT CCTCA | 345 |
| SEQ ID NO: 332 | GACATCTTGC TGACTCAGTC TCCAGCCATC CTGTCTGTGA GTCCAGGAGA AAGAGTCAGT | 60 |
| Anti-TROP-2 | TTCTCCTGCA GGGCCAGTCA GACCATTGGC ACAAGCATAC ACTGGTATCA GCAAAGAACA | 120 |
| m6G11 variable | AATGGTTCTC CAAGGCTTCT CATAAAGTAT GCTTCTGAGT CTASCTCTGG GATCCCTTCC | 180 |
| light chain | AGGTTTAGTG GCAGTGGATC AGGGACAGAT TTTACTCTTA GCASCAACAG TGTGGAGTCT | 240 |
| | GAAGATATTG CAGATTATTA CTGTCAACAA AGTAATAGCT GGCCATTCAC GTTCGGCTCG | 300 |
| | GGGACCAAGC TGGAAATAAA A | 321 |
| SEQ ID NO:333 | CAGGTGCAGT TGGTTCAGAG CGGCGCGGAA GTCAAGAAAC CCGGCGCCTC CGTGAAAGTG | 60 |
| Anti-TROP-2 | AGCTGCAAAG CGAGCGGCTA CACCTTCACC AGTTATTGGA TTAACTGGGT GCGCCAGGCC | 120 |
| h6G11 variable | CCAGGCCAGG GGCTGGAGTG GATGGGAAAC ATCTACCCAT CTGACTCTTA CAGCAACTAT | 180 |
| heavy chain | AATCAGAAAT TTAAGGATCG CGTAACAATG ACCCGTGACA CCAGCACCAG CACTGTTTAC | 240 |
| | ATGGAGCTGA GTTCTCTGCG TTCTGAAGAT ACCGCCGTGT ACTACTGCGC ACGCGGTTCC | 300 |
| | AGTTTCGATT ACTGGGGCCA GGGGACCCTG GTCACCGTCT CCTCA | 345 |
| SEQ ID NO: 334 | GAGATCGTGC TGACCCAAAG TCCAGCCACC CTTTCCCTGT CTCCAGGCGA ACGCGCAACC | 60 |
| Anti-TROP-2 | CTGAGCTGCC GCGCTTCTCA GACCATTGGT ACCTCCATTC ATTGGTATCA GCAGAAGCCC | 120 |
| h6G11 variable | GGCCAAGCCC CGCGTCTGCT GATCTATTAC GCCTCAGAAA GTATTTCAGG CATCCCCGCT | 180 |
| light chain | CGCTTCTCCG GCTCCGGCAG CGGAACCGAC TTCACACTTA CAATCTCTAG TTTGGAGCCA | 240 |
| | GAAGACTTCG CCGTTTACTA CTGTCAGCAG TCTAACAGCT GGCCATTTAC CTTTGGCCAG | 300 |
| | GGCACGAAGC TGGAAATCAA G | 321 |
| SEQ ID NO: 335 | CAGGTGCAGT TGGTTCAGAG CGGCGCGGAA GTCAAGAAAC CCGGCGCCTC CGTGAAAGTG | 60 |
| Anti-TROP-2 | AGCTGCAAAG CGAGCGGCTA CACCTTCACC AGTTATTGGA TTAACTGGGT GCGCCAGGCC | 120 |
| h6G11-FKG SF | CCAGGCCAGG GGCTGGAGTG GATGGGAAAC ATCTTCCCAT CTGACTCTTA CAGCAACTAT | 180 |
| variable heavy | AATAAGAAAT TTAAGGATCG CGTAACAATG ACCCGTGACA CCAGCACCAG CACTGTTTAC | 240 |
| chain | ATGGAGCTGA GTTCTCTGCG TTCTGAAGAT ACCGCCGTGT ACTACTGCGC ACGCGGTTCC | 300 |
| | GGGTTCGATT ACTGGGGCCA GGGGACCCTG GTCACCGTCT CCTCA | 345 |
| SEQ ID NO: 336 | GAGATCGTGC TGACCCAAAG TCCAGCCACC CTTTCCCTGT CTCCAGGCGA ACGCGCAACC | 60 |
| Anti-TROP-2 | CTGAGCTGCC GCGCTTCTCA GACCATTGGT ACCTCCATTC ATTGGTATCA GCAGAAGCCC | 120 |
| h6G11-FKG SF | GGCCAAGCCC CGCGTCTGCT GATCTATTAC GCCTCAGAAA GTATTTCAGG CATCCCCGCT | 180 |
| variable light | CGCTTCTCCG GCTCCGGCAG CGGAACCGAC TTCACACTTA CAATCTCTAG TTTGGAGCCA | 240 |
| chain | GAAGACTTCG CCGTTTACTA CTGTTCGCAG TCTTTTAGCT GGCCATTTAC CTTTGGCCAG | 300 |
| | GGCACGAAGC TGGAAATCAA G | 321 |

In an embodiment, an anti-TROP-2 scFv domain comprises a heavy chain variable region (V_{H}) domain and/or a light chain variable region (V_{L}) domain of the scFv antibody m7E6 encoded by a nucleotide sequence. In an embodiment, an anti-TROP-2 scFv domain comprises a V_{H} domain and/or a V_{L} domain, wherein the V_{H} domain is encoded by the sequence shown in SEQ ID NO:325 and the light chain variable region (V_{L}) is encoded by the sequence shown in SEQ ID NO:326. In an embodiment, an anti-TROP-2 scFv domain comprises V_{H} and/or V_{L} regions that are encoded by nucleotides that are each at least 99% identical to the sequences shown in SEQ ID NO:325 and SEQ ID NO:326, respectively. In an embodiment, an anti-TROP-2 scFv domain comprises V_{H} and/or V_{L} regions that are encoded by nucleotides that are each at least 98% identical to the sequences shown in SEQ ID NO:325 and SEQ ID NO:326, respectively. In an embodiment, an anti-TROP-2 scFv domain comprises V_{H} and/or V_{L} regions that are encoded by nucleotides that are each at least 97% identical to the sequences shown in SEQ ID NO:325 and SEQ ID NO:326, respectively. In an embodiment, an anti-TROP-2 scFv domain comprises V_{H} and/or V_{L} regions that are encoded by nucleotides that are each at least 96% identical to the sequences shown in SEQ ID NO:325 and SEQ ID NO:326, respectively. In an embodiment, an anti-TROP-2 scFv domain comprises V_{H} and/or V_{L} regions that are encoded by nucleotides that are each at least 95% identical to the sequences shown in SEQ ID NO:325 and SEQ ID NO:326, respectively. In an embodiment, an anti-TROP-2 scFv domain comprises V_{H} and/or V_{L} regions that are encoded by nucleotides that are each at least 90% identical to the sequences shown in SEQ ID NO:325 and SEQ ID NO:326, respectively. In an embodiment, an anti-TROP-2 scFv domain comprises V_{H} and/or V_{L} regions that are encoded by nucleotides that are each at least 85% identical to the sequences shown in SEQ ID NO:325 and SEQ ID NO:326, respectively. In an embodiment, an anti-TROP-2 scFv domain comprises V_{H} and/or V_{L} regions that are encoded by nucleotides that are each at least 80% identical to the sequences shown in SEQ ID NO:325 and SEQ ID NO:326, respectively. In an embodiment, including the foregoing embodiments, SEQ ID NO:325 and/or SEQ ID NO:326 is codon-optimized to improve protein expression.

In an embodiment, an anti-TROP-2 scFv domain comprises a heavy chain variable region (V_{H}) domain and/or a light chain variable region (V_{L}) domain of the scFv antibody h7E6 encoded by a nucleotide sequence. In an embodiment, an anti-TROP-2 scFv domain comprises a V_{H} domain and/or a V_{L} domain, wherein the V_{H} domain is encoded by the sequence shown in SEQ ID NO:327 and the light chain variable region (V_{L}) is encoded by the sequence shown in SEQ ID NO:328. In an embodiment, an anti-TROP-2 scFv domain comprises V_{H} and/or V_{L} regions that are encoded by nucleotides that are each at least 99% identical to the sequences shown in SEQ ID NO:327 and SEQ ID NO:328, respectively. In an embodiment, an anti-TROP-2 scFv domain comprises V_{H} and/or V_{L} regions that are encoded by nucleotides that are each at least 98% identical to the sequences shown in SEQ ID NO:327 and SEQ ID NO:328, respectively. In an embodiment, an anti-TROP-2 scFv domain comprises V_{H} and/or V_{L} regions that are encoded by nucleotides that are each at least 97% identical to the sequences shown in SEQ ID NO:327 and SEQ ID NO:328, respectively. In an embodiment, an anti-TROP-2 scFv domain comprises V_{H} and/or V_{L} regions that are encoded by nucleotides that are each at least 96% identical to the sequences shown in SEQ ID NO:327 and SEQ ID NO:328, respectively. In an embodiment, an anti-TROP-2 scFv domain comprises V_{H} and/or V_{L} regions that are encoded by nucleotides that are each at least 95% identical to the sequences shown in SEQ ID NO:327 and SEQ ID NO:328, respectively. In an embodiment, an anti-TROP-2 scFv domain comprises V_{H} and/or V_{L} regions that are encoded by nucleotides that are each at least 90% identical to the sequences shown in SEQ ID NO:327 and SEQ ID NO:328, respectively. In an embodiment, an anti-TROP-2 scFv domain comprises V_{H} and/or V_{L} regions that are encoded by nucleotides that are each at least 85% identical to the sequences shown in SEQ ID NO:327 and SEQ ID NO:328, respectively. In an embodiment, an anti-TROP-2 scFv domain comprises V_{H} and/or V_{L} regions that are encoded by nucleotides that are each at least 80% identical to the sequences shown in SEQ ID NO:327 and SEQ ID NO:328, respectively. In an embodiment, including the foregoing embodiments, SEQ ID NO:327 and/or SEQ ID NO:328 is codon-optimized to improve protein expression.

In an embodiment, an anti-TROP-2 scFv domain comprises a heavy chain variable region (V_{H}) domain and/or a light chain variable region (V_{L}) domain of the scFv antibody h7E6_SVG encoded by a nucleotide sequence. In an embodiment, an anti-TROP-2 scFv domain comprises a V_{H} domain and/or a V_{L} domain, wherein the V_{H} domain is encoded by the sequence shown in SEQ ID NO:329 and the light chain variable region (V_{L}) is encoded by the sequence shown in SEQ ID NO:328. In an embodiment, an anti-TROP-2 scFv domain comprises V_{H} and/or V_{L} regions that are encoded by nucleotides that are each at least 99% identical to the sequences shown in SEQ ID NO:329 and SEQ ID NO:328, respectively. In an embodiment, an anti-TROP-2 scFv domain comprises V_{H} and/or V_{L} regions that are encoded by nucleotides that are each at least 98% identical to the sequences shown in SEQ ID NO:329 and SEQ ID NO:328, respectively. In an embodiment, an anti-TROP-2 scFv domain comprises V_{H} and/or V_{L} regions that are encoded by nucleotides that are each at least 97% identical to the sequences shown in SEQ ID NO:329 and SEQ ID NO:328, respectively. In an embodiment, an anti-TROP-2 scFv domain comprises V_{H} and/or V_{L} regions that are encoded by nucleotides that are each at least 96% identical to the sequences shown in SEQ ID NO:329 and SEQ ID NO:328, respectively. In an embodiment, an anti-TROP-2 scFv domain comprises V_{H} and/or V_{L} regions that are encoded by nucleotides that are each at least 95% identical to the sequences shown in SEQ ID NO:329 and SEQ ID NO:328, respectively. In an embodiment, an anti-TROP-2 scFv domain comprises V_{H} and/or V_{L} regions that are encoded by nucleotides that are each at least 90% identical to the sequences shown in SEQ ID NO:329 and SEQ ID NO:328, respectively. In an embodiment, an anti-TROP-2 scFv domain comprises V_{H} and/or V_{L} regions that are encoded by nucleotides that are each at least 85% identical to the sequences shown in SEQ ID NO:329 and SEQ ID NO:328, respectively. In an embodiment, an anti-TROP-2 scFv domain comprises V_{H} and/or V_{L} regions that are encoded by nucleotides that are each at least 80% identical to the sequences shown in SEQ ID NO:329 and SEQ ID NO:328, respectively. In an embodiment, including the foregoing embodiments, a nucleotide sequence in Table 43 is codon-optimized to improve protein expression. In an embodiment, including the foregoing embodiments, SEQ ID NO:329 and/or SEQ ID NO:328 is codon-optimized to improve protein expression.

In an embodiment, an anti-TROP-2 scFv domain comprises a heavy chain variable region (V_{H}) domain and/or a light chain variable region (V_{L}) domain of the scFv antibody h7E6_SVGL encoded by a nucleotide sequence. In an embodiment, an anti-TROP-2 scFv domain comprises a V_{H} domain and/or a V_{L} domain, wherein the V_{H} domain is encoded by the sequence shown in SEQ ID NO:329 and the light chain variable region (V_{L}) is encoded by the sequence shown in SEQ ID NO:330. In an embodiment, an anti-TROP-2 scFv domain comprises V_{H} and/or V_{L} regions that are encoded by nucleotides that are each at least 99% identical to the sequences shown in SEQ ID NO:329 and SEQ ID NO:330, respectively. In an embodiment, an anti-TROP-2 scFv domain comprises V_{H} and/or V_{L} regions that are encoded by nucleotides that are each at least 98% identical to the sequences shown in SEQ ID NO:329 and SEQ ID NO:330, respectively. In an embodiment, an anti-TROP-2 scFv domain comprises V_{H} and/or V_{L} regions that are encoded by nucleotides that are each at least 97% identical to the sequences shown in SEQ ID NO:329 and SEQ ID NO:330, respectively. In an embodiment, an anti-TROP-2 scFv domain comprises V_{H} and/or V_{L} regions that are encoded by nucleotides that are each at least 96% identical to the sequences shown in SEQ ID NO:329 and SEQ ID NO:330, respectively. In an embodiment, an anti-TROP-2 scFv domain comprises V_{H} and/or V_{L} regions that are encoded by nucleotides that are each at least 95% identical to the sequences shown in SEQ ID NO:329 and SEQ ID NO:330, respectively. In an embodiment, an anti-TROP-2 scFv domain comprises V_{H} and/or V_{L} regions that are encoded by nucleotides that are each at least 90% identical to the sequences shown in SEQ ID NO:329 and SEQ ID NO:330, respectively. In an embodiment, an anti-TROP-2 scFv domain comprises V_{H} and/or V_{L} regions that are encoded by nucleotides that are each at least 85% identical to the sequences shown in SEQ ID NO:329 and SEQ ID NO:330, respectively. In an embodiment, an anti-TROP-2 scFv domain comprises V_{H} and/or V_{L} regions that are encoded by nucleotides that are each at least 80% identical to the sequences shown in SEQ ID NO:329 and SEQ ID NO:330, respectively. In an embodiment, including the foregoing embodiments, SEQ ID NO:329 and/or SEQ ID NO:330 is codon-optimized to improve protein expression.

In an embodiment, an anti-TROP-2 scFv domain comprises a heavy chain variable region (V_{H}) domain and/or a light chain variable region (V_{L}) domain of the scFv antibody m6G11 encoded by a nucleotide sequence. In an embodiment, an anti-TROP-2 scFv domain comprises a V_{H} domain and/or a V_{L} domain, wherein the V_{H} domain is encoded by the sequence shown in SEQ ID NO:331 and the light chain variable region (V_{L}) is encoded by the sequence shown in SEQ ID NO:332. In an embodiment, an anti-TROP-2 scFv domain comprises V_{H} and/or V_{L} regions that are encoded by nucleotides that are each at least 99% identical to the sequences shown in SEQ ID NO:331 and SEQ ID NO:332, respectively. In an embodiment, an anti-TROP-2 scFv domain comprises V_{H} and/or V_{L} regions that are encoded by nucleotides that are each at least 98% identical to the sequences shown in SEQ ID NO:331 and SEQ ID NO:332, respectively. In an embodiment, an anti-TROP-2 scFv domain comprises V_{H} and/or V_{L} regions that are encoded by nucleotides that are each at least 97% identical to the sequences shown in SEQ ID NO:331 and SEQ ID NO:332, respectively. In an embodiment, an anti-TROP-2 scFv domain comprises V_{H} and/or V_{L} regions that are encoded by nucleotides that are each at least 96% identical to the sequences shown in SEQ ID NO:331 and SEQ ID NO:332, respectively. In an embodiment, an anti-TROP-2 scFv domain comprises V_{H} and/or V_{L} regions that are encoded by nucleotides that are each at least 95% identical to the sequences shown in SEQ ID NO:331 and SEQ ID NO:332, respectively. In an embodiment, an anti-TROP-2 scFv domain comprises V_{H} and/or V_{L} regions that are encoded by nucleotides that are each at least 90% identical to the sequences shown in SEQ ID NO:331 and SEQ ID NO:332, respectively. In an embodiment, an anti-TROP-2 scFv domain comprises V_{H} and/or V_{L} regions that are encoded by nucleotides that are each at least 85% identical to the sequences shown in SEQ ID NO:331 and SEQ ID NO:332, respectively. In an embodiment, an anti-TROP-2 scFv domain comprises V_{H} and/or V_{L} regions that are encoded by nucleotides that are each at least 80% identical to the sequences shown in SEQ ID NO:331 and SEQ ID NO:332, respectively. In an embodiment, including the foregoing embodiments, SEQ ID NO:331 and/or SEQ ID NO:332 is codon-optimized to improve protein expression.

In an embodiment, an anti-TROP-2 scFv domain comprises a heavy chain variable region (V_{H}) domain and/or a light chain variable region (V_{L}) domain of the scFv antibody h6G11 encoded by a nucleotide sequence. In an embodiment, an anti-TROP-2 scFv domain comprises a V_{H} domain and/or a V_{L} domain, wherein the V_{H} domain is encoded by the sequence shown in SEQ ID NO:333 and the light chain variable region (V_{L}) is encoded by the sequence shown in SEQ ID NO:334. In an embodiment, an anti-TROP-2 scFv domain comprises V_{H} and/or V_{L} regions that are encoded by nucleotides that are each at least 99% identical to the sequences shown in SEQ ID NO:333 and SEQ ID NO:334, respectively. In an embodiment, an anti-TROP-2 scFv domain comprises V_{H} and/or V_{L} regions that are encoded by nucleotides that are each at least 98% identical to the sequences shown in SEQ ID NO:333 and SEQ ID NO:334, respectively. In an embodiment, an anti-TROP-2 scFv domain comprises V_{H} and/or V_{L} regions that are encoded by nucleotides that are each at least 97% identical to the sequences shown in SEQ ID NO:333 and SEQ ID NO:334, respectively. In an embodiment, an anti-TROP-2 scFv domain comprises V_{H} and/or V_{L} regions that are encoded by nucleotides that are each at least 96% identical to the sequences shown in SEQ ID NO:333 and SEQ ID NO:334, respectively. In an embodiment, an anti-TROP-2 scFv domain comprises V_{H} and/or V_{L} regions that are encoded by nucleotides that are each at least 95% identical to the sequences shown in SEQ ID NO:333 and SEQ ID NO:334, respectively. In an embodiment, an anti-TROP-2 scFv domain comprises V_{H} and/or V_{L} regions that are encoded by nucleotides that are each at least 90% identical to the sequences shown in SEQ ID NO:333 and SEQ ID NO:334, respectively. In an embodiment, an anti-TROP-2 scFv domain comprises V_{H} and/or V_{L} regions that are encoded by nucleotides that are each at least 85% identical to the sequences shown in SEQ ID NO:333 and SEQ ID NO:334, respectively. In an embodiment, an anti-TROP-2 scFv domain comprises V_{H} and/or V_{L} regions that are encoded by nucleotides that are each at least 80% identical to the sequences shown in SEQ ID NO:333 and SEQ ID NO:334, respectively. In an embodiment, including the foregoing embodiments, SEQ ID NO:333 and/or SEQ ID NO:334 is codon-optimized to improve protein expression.

In an embodiment, an anti-TROP-2 scFv domain comprises a heavy chain variable region (V_{H}) domain and/or a light chain variable region (V_{L}) domain of the scFv antibody h6G11-FKG_SF encoded by a nucleotide sequence. In an embodiment, an anti-TROP-2 scFv domain comprises a V_{H} domain and/or a V_{L} domain, wherein the V_{H} domain is encoded by the sequence shown in SEQ ID NO:335 and the light chain variable region (V_{L}) is encoded by the sequence shown in SEQ ID NO:336. In an embodiment, an anti-TROP-2 scFv domain comprises V_{H} and/or V_{L} regions that are encoded by nucleotides that are each at least 99% identical to the sequences shown in SEQ ID NO:335 and SEQ ID NO:336, respectively. In an embodiment, an anti-TROP-2 scFv domain comprises V_{H} and/or V_{L} regions that are encoded by nucleotides that are each at least 98% identical to the sequences shown in SEQ ID NO:335 and SEQ ID NO:336, respectively. In an embodiment, an anti-TROP-2 scFv domain comprises V_{H} and/or V_{L} regions that are encoded by nucleotides that are each at least 97% identical to the sequences shown in SEQ ID NO:335 and SEQ ID NO:336, respectively. In an embodiment, an anti-TROP-2 scFv domain comprises V_{H} and/or V_{L} regions that are encoded by nucleotides that are each at least 96% identical to the sequences shown in SEQ ID NO:335 and SEQ ID NO:336, respectively. In an embodiment, an anti-TROP-2 scFv domain comprises V_{H} and/or V_{L} regions that are encoded by nucleotides that are each at least 95% identical to the sequences shown in SEQ ID NO:335 and SEQ ID NO:336, respectively. In an embodiment, an anti-TROP-2 scFv domain comprises V_{H} and/or V_{L} regions that are encoded by nucleotides that are each at least 90% identical to the sequences shown in SEQ ID NO:335 and SEQ ID NO:336, respectively. In an embodiment, an anti-TROP-2 scFv domain comprises V_{H} and/or V_{L} regions that are encoded by nucleotides that are each at least 85% identical to the sequences shown in SEQ ID NO:335 and SEQ ID NO:336, respectively. In an embodiment, an anti-TROP-2 scFv domain comprises V_{H} and/or V_{L} regions that are encoded by nucleotides that are each at least 80% identical to the sequences shown in SEQ ID NO:335 and SEQ ID NO:336, respectively. In an embodiment, including the foregoing embodiments, SEQ ID NO:335 and/or SEQ ID NO:336 is codon-optimized to improve protein expression.

In an embodiment, a CCR comprises an extracellular scFv domain that binds to TROP-2 and comprises V_{H}, V_{L}, or CDR domains of sacituzumab, or a fragment, derivative, or variant thereof. The preparation and properties of sacituzumab, an anti-TROP-2 monoclonal antibody, and its V_{H}, V_{L}, CDR and other relevant domains, including amino acid and nucleotide sequences thereof, is described in U.S. Pat. No. 9,770,517, the disclosures of which are incorporated by reference herein. The amino acid sequences of exemplary TROP-2 binding domains for use in the CCRs of the present invention are provided in Table 44.

TABLE 44
Amino acid sequences of sacituzumab TROP-2 binding scFv domains.

| Identifier | Sequence (One-Letter Amino Acid Symbols) | |
|---|---|---|
| SEQ ID NO: 337 | QVQLCQSGSE LKKPGASVKV SCKASGYTFT NYGMNWVKQA PGQGLKWMGW INTYTGEPTY | 60 |
| Anti-TROP-2 | TDDFKGRFAF SLDTSVSTAY LQISSLKADD TAVYFCARGG FGSSYWYFDV WGQGSLVTVS | 120 |
| sacituzumab | | |
| variable heavy | | |
| chain | | |
| SEQ ID NO: 338 | DIQLTQSPSS LSASVGDRVS ITCKASQDVS IAVAWYQQKP GKAPKLLIYS ASYRYTGVPD | 60 |
| Anti-TROP-2 | RFSGSGSGTD FTLTISSLQP EDFAVYYCQQ HYITPLTFGA GTKVEIK | 107 |
| sacituzumab | | |
| variable light | | |
| chain | | |
| SEQ ID NO: 339 | NYGMN | 5 |
| Anti-TROP-2 | | |
| sacituzumab CDR1 | | |
| heavy chain | | |
| SEQ ID NO: 340 | WINTYTGEPT YTDDFKG | 17 |
| Anti-TROP-2 | | |
| sacituzumab CDR2 | | |
| heavy chain | | |
| SEQ ID NO: 341 | GGFGSSYWYF DV | 12 |
| Anti-TROP-2 | | |
| sacituzumab CDR3 | | |
| heavy chain | | |
| SEQ ID NO: 342 | KASQDVSIAV A | 11 |
| Anti-TROP-2 | | |
| sacituzumab CDR1 | | |
| light chain | | |
| SEQ ID NO: 343 | SASYRYT | 7 |
| Anti-TROP-2 | | |
| sacituzumab CDR2 | | |
| light chain | | |
| SEQ ID NO: 344 | QQHYITPLT | 9 |
| Anti-TROP-2 | | |
| sacituzumab CDR3 | | |
| light chain | | |

In an embodiment, an anti-TROP-2 scFv domain comprises a V_{H} domain and/or a V_{L} domain of sacituzumab, or a fragment, variant, derivative, or biosimilar thereof. In an embodiment, an anti-TROP-2 scFv domain comprises a V_{H} domain and/or a V_{L} domain, wherein the V_{H} domain comprises the sequence shown in SEQ ID NO:337, or conservative amino acid substitutions thereof, and the light chain variable region (V_{L}) comprises the sequence shown in SEQ ID NO:338, or conservative amino acid substitutions thereof. In an embodiment, an anti-TROP-2 scFv domain comprises V_{H} and/or V_{L} regions that are each at least 99% identical to the sequences shown in SEQ ID NO:337 and/or SEQ ID NO:338, respectively. In an embodiment, an anti-TROP-2 scFv domain comprises V_{H} and/or V_{L} regions that are each at least 98% identical to the sequences shown in SEQ ID NO:337 and/or SEQ ID NO:338, respectively. In an embodiment, an anti-TROP-2 scFv domain comprises V_{H} and/or V_{L} regions that are each at least 97% identical to the sequences shown in SEQ ID NO:337 and/or SEQ ID NO:338, respectively. In an embodiment, an anti-TROP-2 scFv domain comprises V_{H} and/or V_{L} regions that are each at least 96% identical to the sequences shown in SEQ ID NO:337 and/or SEQ ID NO:338, respectively. In an embodiment, an anti-TROP-2 scFv domain comprises V_{H} and/or V_{L} regions that are each at least 95% identical to the sequences shown in SEQ ID NO:337 and/or SEQ ID NO:338, respectively. In an embodiment, an anti-TROP-2 scFv domain comprises V_{H} and/or V_{L} regions that are each at least 90% identical to the sequences shown in SEQ ID NO:337 and/or SEQ ID NO:338, respectively. In an embodiment, an anti-TROP-2 scFv domain comprises V_{H} and/or V_{L} regions that are each at least 85% identical to the sequences shown in SEQ ID NO:337 and/or SEQ ID NO:338, respectively. In an embodiment, an anti-TROP-2 scFv domain comprises V_{H} and/or V_{L} regions that are each at least 80% identical to the sequences shown in SEQ ID NO:337 and/or SEQ ID NO:338, respectively.

In an embodiment, an anti-TROP-2 scFv domain comprises the heavy chain CDR1, CDR2 and/or CDR3 domains of sacituzumab, or conservative amino acid substitutions thereof, and/or light chain CDR1, CDR2 and CDR3 domains of sacituzumab, or conservative amino acid substitutions thereof. In an embodiment, an anti-TROP-2 scFv domain comprises the heavy chain CDR1, CDR2 and CDR3 domains having the sequences set forth in SEQ ID NO:339, SEQ ID NO:340, and/or SEQ ID NO:341, respectively, or conservative amino acid substitutions thereof, and/or light chain CDR1, CDR2 and CDR3 domains having the sequences set forth in SEQ ID NO:342, SEQ ID NO:343, and/or SEQ ID NO:344, respectively, or conservative amino acid substitutions thereof.

In an embodiment, a CCR comprises an extracellular scFv domain, or V_{H} and/or V_{L} or heavy chain and/or light chain CDR1, CDR2, and/or CDR3 domains, which binds to TROP-2 and is disclosed in U.S. Pat. Nos. 9,062,100; 9,670,287; 9,850,312; and 10,202,461; and U.S. Patent Application Publication No. US 2019/0144559 A1, the disclosures of which are incorporated by reference herein.

In an embodiment, a CCR comprises an extracellular scFv domain, or V_{H} and/or V_{L} or heavy chain and/or light chain CDR1, CDR2, and/or CDR3 domains, which binds to TROP-2 from antibody AR47A6.4.2 disclosed in U.S. Patent Application Publication No. US 2008/0131428 A1, the disclosures of which are incorporated by reference herein

### 6. Extracellular EPCAM Binding Domains

In an embodiment, a CCR comprises an extracellular domain, wherein the extracellular domain is a domain capable of binding to EPCAM, which is also known as epithelial cell adhesion molecule, tumor-associated calcium signal transducer 1 or TACSTD1, CD326, and 17-A1 antigen. A domain capable of binding to EPCAM is also referred to herein as an anti-EPCAM domain. In an embodiment, the extracellular domain binds to human EPCAM. In an embodiment, the extracellular domain binds to murine EPCAM. In an embodiment, the extracellular EPCAM binding domain is a scFv domain that binds to human EPCAM or murine EPCAM. In an embodiment, a CCR of the present invention comprises a construct as shown in FIG. 34, wherein the V_{H} and V_{L} domains are anti-EPCAM V_{H} and V_{L} domains, and the linker is as described herein.

In some embodiments, the EPCAM binding domain includes the scFv domain for antibodies 3-17I scFv, 7-F17 scFv, 12-C15 scFv, 16-G5 scFv, 17-C20 scFv, and 24-G6 scFv, and fragments, variants, and derivatives thereof, each as described in U.S. Pat. No. 8,637,017, the disclosures of which are incorporated by reference herein. The amino acid sequences of exemplary EPCAM binding domains for use in the CCRs of the present invention are provided in Table 45.

TABLE 45
Amino acid sequences of exemplary EPCAM binding scFv domains.

| Identifier | Sequence (One-Letter Amino Acid Symbols) | |
|---|---|---|
| SEQ ID NO: 345 | QVQLVQSGAE VKKPGSSVKV SCKASGGTFS SYAISWVRQA PGQGLEWMGG IIPIFGTANY | 60 |
| Anti-EPCAM 3-17I | AQKFQGRVTI TADESTSTAY MELSSLRSED TAVYYCARGL LWNYWGQGTL VTVSSKLSGS | 120 |
| scFv | ASAPKLEEGE FSEARVEIVM TQSPATLSVS PGERATLSCR ASQSVSSNLA WYQQKPGQAP | 180 |
| | RLIIYGASTT ASGIPARFSA SGSGTDFTLT ISSLQSEDFA VYYCQQYNNW PPAYTFGQGT | 240 |
| | KLEIK | 245 |
| SEQ ID NO: 346 | QVQLVQSGAE VKKPGSSVKV SCKASGGTFS SYAISWVRQA PGQGLEWMGG IIPIFGTANY | 60 |
| Anti-EPCAM 7-F17 | AQKFQGRVTI TADESTSTAY MELSSLRSED TAVYYCARGL LWNYWGQGTL VTVSSKLSGS | 120 |
| scFv | ASAPKLEEGE FSEARVETTL TQSPATLSVS PGERATLSCR ASQSVSSNLA WYQQKPGQAP | 180 |
| | RLLIYGASTR ATGIPARFSG SGSGTEFTLT ISSLQSEDFA VYYCQQYNNW PPGFTFGPGT | 240 |
| | KVDIK | 245 |
| SEQ ID NO: 347 | QVQLVQSGAE VKKPGSSVKV SCKASGGTFS SYAISWVRQA PGQGLEWMGG IIPIFGTANY | 60 |
| Anti-EPCAM 12- | AQKFQGRVTI TADESTSTAY MELSSLRSED TAVYYCARGL LWNYWGQGTL VTVSSKLSGS | 120 |
| C15 scFv | ASAPKLEEGE FSEARVETTL TQSPATLSLS PGERATLSCR ASQSVSSNLA WYQQKPGQAP | 180 |
| | RLLIYGASTR ATGIPARFSG SGSGTEFTLT ISSLQSEDFA VYYCQHYNDW PPTWTFGQGT | 240 |
| | KLEIK | 245 |
| SEQ ID NO: 348 | QVQLVQSGAE VKKPGSSVKV SCKASGGTFS SYAISWVRQA PGQGLEWMGG IIPIFGTANY | 60 |
| Anti-EPCAM 16-G5 | AQKFQGRVTI TADESTSTAY MELSSLRSED TAVYYCARGL LWNYWGQGTL VTVSSKLSGS | 120 |
| scFv | ASAPKLEEGE FSEARVDIVM TQTPATLSVS PGERATLSCR ASQSVSSNLA WYQQKPGQAP | 180 |
| | RLLIYGASTR ATGIPARFSG SGSGTEFTLT ISSLQSEDFA VYYCQQYNNW PPSWTFGQGT | 240 |
| | KVEIK | 245 |
| SEQ ID NO: 349 | QVQLVQSGAE VKKPGSSVKV SCKASGGTFS SYAISWVRQA PGQGLEWMGG IIPIFGTANY | 60 |
| Anti-EPCAM 17- | AQKFQGRVTI TADESTSTAY MELSSLRSED TAVYYCARGL LWNYWGQGTL VTVSSKLSGS | 120 |
| C20 scFv | ASAPKLEEGE FSEARVETTL TQSPATLSVS PGERATLSCR ASQSVSSNLA WYQQKPGQAP | 180 |
| | RLLIYGASTR ATGIPARFSG SGSGTEFTLT ISSLQSEDFA VYYCQQYNNW PPMYTFGQGT | 240 |
| | KVEIK | 245 |
| SEQ ID NO: 350 | QVQLVQSGAE VKKPGSSVKV SCKASGGTFS SYAISWVRQA PGQGLEWMGG IIPIFGTANY | 60 |
| Anti-EPCAM 24-G6 | AQKFQGRVTI TADESTSTAY MELSSLRSED TAVYYCARGL LWNYWGQGTL VTVSSKLSGS | 120 |
| scFv | ASAPKLEEGE FSEARVETTL TQSPATLSVS PGERATLSCR ASQSVSSNLA WYQQKPGQAP | 180 |
| | RLLIYGASTR ATGIPARFSG SGSGTEFTLT ISSLQSEDFA VYYCQKYNNW PPAFTFGPGT | 240 |
| | KVDIK | 245 |
| SEQ ID NO: 351 | QVQLVQSGAE VKKPGSSVKV SCKASGGTFS SYAISWVRQA PGQGLEWMGG IIPIFGTANY | 60 |
| Anti-EPCAM | AQKFQGRVTI TADESTSTAY MELSSLRSED TAVYYCARGL LWNYWGQGTL VTVSS | 115 |
| variable heavy | | |
| chain | | |
| SEQ ID NO: 352 | EIVMTQSPAT LSVSPGERAT LSCRASQSVS SNLAWYQQKP GQAPRLIIYG ASTTASGIPA | 60 |
| Anti-EPCAM | RFSASGSGTD FTLTISSLQS EDFAVYYCQQ YNNWPPAYTF GQGTKLEIK | 109 |
| variable light | | |
| chain | | |
| SEQ ID NO: 353 | ETTLTQSPAT LSVSPGERAT LSCRASQSVS SNLAWYQQKP GQAPRLLIYG ASTRATGIPA | 60 |
| Anti-EPCAM | RFSGSGSGTE FTLTISSLQS EDFAVYYCQQ YNNWPPGFTF GPGTKVDIK | 109 |
| variable light | | |
| chain | | |
| SEQ ID NO: 354 | ETTLTQSPAT LSLSPGERAT LSCRASQSVS SNLAWYQQKP GQAPRLLIYG ASTRATGIPA | 60 |
| Anti-EPCAM | RFSGSGSGTE FTLTISSLQS EDFAVYYCQH YNDWPPTWTF GQGTKLEIK | 109 |
| variable light | | |
| chain | | |
| SEQ ID NO: 355 | DIVMTQTPAT LSVSPGERAT LSCRASQSVS SNLAWYQQKP GQAPRLLIYG ASTRATGIPA | 60 |
| Anti-EPCAM | RFSGSGSGTE FTLTISSLQS EDFAVYYCQQ YNNWPPSWTF GQGTKVEIK | 109 |
| variable light | | |
| chain | | |
| SEQ ID NO: 356 | ETTLTQSPAT LSVSPGERAT LSCRASQSVS SNLAWYQQKP GQAPRLLIYG ASTRATGIPA | 6 0 |
| Anti-EPCAM | RFSGSGSGTE FTLTISSLQS EDFAVYYCQQ YNNWPPMYTF GQGTKVEIK | 109 |
| variable light | | |
| chain | | |
| SEQ ID NO: 357 | ETTLTQSPAT LSVSPGERAT LSCRASQSVS SNLAWYQQKP GQAPRLLIYG ASTRATGIPA | 60 |
| Anti-EPCAM | RFSGSGSGTE FTLTISSLQS EDFAVYYCQK YNNWPPAFTF GPGTKVDIK | 109 |
| variable light | | |
| chain | | |
| SEQ ID NO: 358 | SYAIS | 5 |
| Anti-EPCAM CDR1 | | |
| heavy chain | | |
| SEQ ID NO: 359 | GIIPIFGTAN YAQKFQG | 17 |
| Anti-EPCAM CDR2 | | |
| heavy chain | | |
| SEQ ID NO: 360 | GLLWNY | 6 |
| Anti-EPCMA CDR3 | | |
| heavy chain | | |
| SEQ ID NO: 361 | RASQSVSSNL A | 11 |
| Anti-EPCAM CDR1 | | |
| light chain | | |
| SEQ ID NO: 362 | GASTTAS | 7 |
| Anti-EPCAM CDR2 | | |
| light chain | | |
| SEQ ID NO: 363 | QQYNNWPPAY T | 11 |
| Anti-EPCAM CDR3 | | |
| light chain | | |

In an embodiment, an anti-EPCAM scFv domain comprises the sequence shown in SEQ ID NO:345, or conservative amino acid substitutions thereof. In an embodiment, an anti-EPCAM scFv domain comprises the scFv antibody 3-17I scFv, or conservative amino acid substitutions thereof. In an embodiment, an anti-EPCAM scFv domain comprises a scFv domain that is at least 99% identical to the sequence shown in SEQ ID NO:345. In an embodiment, an anti-EPCAM scFv domain comprises a scFv domain that is at least 98% identical to the sequence shown in SEQ ID NO:345. In an embodiment, an anti-EPCAM scFv domain comprises a scFv domain that is at least 97% identical to the sequence shown in SEQ ID NO:345. In an embodiment, an anti-EPCAM scFv domain comprises a scFv domain that is at least 96% identical to the sequence shown in SEQ ID NO:345. In an embodiment, an anti-EPCAM scFv domain comprises a scFv domain that is at least 95% identical to the sequence shown in SEQ ID NO:345. In an embodiment, an anti-EPCAM scFv domain comprises a scFv domain that is at least 90% identical to the sequence shown in SEQ ID NO:345. In an embodiment, an anti-EPCAM scFv domain comprises a scFv domain that is at least 85% identical to the sequence shown in SEQ ID NO:345. In an embodiment, an anti-EPCAM scFv domain comprises a scFv domain that is at least 80% identical to the sequence shown in SEQ ID NO:345.

In an embodiment, an anti-EPCAM scFv domain comprises the sequence shown in SEQ ID NO:346, or conservative amino acid substitutions thereof. In an embodiment, an anti-EPCAM scFv domain comprises the scFv antibody 7-F17 scFv, or conservative amino acid substitutions thereof. In an embodiment, an anti-EPCAM scFv domain comprises a scFv domain that is at least 99% identical to the sequence shown in SEQ ID NO:346. In an embodiment, an anti-EPCAM scFv domain comprises a scFv domain that is at least 98% identical to the sequence shown in SEQ ID NO:346. In an embodiment, an anti-EPCAM scFv domain comprises a scFv domain that is at least 97% identical to the sequence shown in SEQ ID NO:346. In an embodiment, an anti-EPCAM scFv domain comprises a scFv domain that is at least 96% identical to the sequence shown in SEQ ID NO:346. In an embodiment, an anti-EPCAM scFv domain comprises a scFv domain that is at least 95% identical to the sequence shown in SEQ ID NO:346. In an embodiment, an anti-EPCAM scFv domain comprises a scFv domain that is at least 90% identical to the sequence shown in SEQ ID NO:346. In an embodiment, an anti-EPCAM scFv domain comprises a scFv domain that is at least 85% identical to the sequence shown in SEQ ID NO:346. In an embodiment, an anti-EPCAM scFv domain comprises a scFv domain that is at least 80% identical to the sequence shown in SEQ ID NO:346.

In an embodiment, an anti-EPCAM scFv domain comprises the sequence shown in SEQ ID NO:347, or conservative amino acid substitutions thereof. In an embodiment, an anti-EPCAM scFv domain comprises the scFv antibody 12-C15 scFv, or conservative amino acid substitutions thereof. In an embodiment, an anti-EPCAM scFv domain comprises a scFv domain that is at least 99% identical to the sequence shown in SEQ ID NO:347. In an embodiment, an anti-EPCAM scFv domain comprises a scFv domain that is at least 98% identical to the sequence shown in SEQ ID NO:347. In an embodiment, an anti-EPCAM scFv domain comprises a scFv domain that is at least 97% identical to the sequence shown in SEQ ID NO:347. In an embodiment, an anti-EPCAM scFv domain comprises a scFv domain that is at least 96% identical to the sequence shown in SEQ ID NO:347. In an embodiment, an anti-EPCAM scFv domain comprises a scFv domain that is at least 95% identical to the sequence shown in SEQ ID NO:347. In an embodiment, an anti-EPCAM scFv domain comprises a scFv domain that is at least 90% identical to the sequence shown in SEQ ID NO:347. In an embodiment, an anti-EPCAM scFv domain comprises a scFv domain that is at least 85% identical to the sequence shown in SEQ ID NO:347. In an embodiment, an anti-EPCAM scFv domain comprises a scFv domain that is at least 80% identical to the sequence shown in SEQ ID NO:347.

In an embodiment, an anti-EPCAM scFv domain comprises the sequence shown in SEQ ID NO:348, or conservative amino acid substitutions thereof. In an embodiment, an anti-EPCAM scFv domain comprises the scFv antibody 16-G5 scFv, or conservative amino acid substitutions thereof. In an embodiment, an anti-EPCAM scFv domain comprises a scFv domain that is at least 99% identical to the sequence shown in SEQ ID NO:348. In an embodiment, an anti-EPCAM scFv domain comprises a scFv domain that is at least 98% identical to the sequence shown in SEQ ID NO:348. In an embodiment, an anti-EPCAM scFv domain comprises a scFv domain that is at least 97% identical to the sequence shown in SEQ ID NO:348. In an embodiment, an anti-EPCAM scFv domain comprises a scFv domain that is at least 96% identical to the sequence shown in SEQ ID NO:348. In an embodiment, an anti-EPCAM scFv domain comprises a scFv domain that is at least 95% identical to the sequence shown in SEQ ID NO:348. In an embodiment, an anti-EPCAM scFv domain comprises a scFv domain that is at least 90% identical to the sequence shown in SEQ ID NO:348. In an embodiment, an anti-EPCAM scFv domain comprises a scFv domain that is at least 85% identical to the sequence shown in SEQ ID NO:348. In an embodiment, an anti-EPCAM scFv domain comprises a scFv domain that is at least 80% identical to the sequence shown in SEQ ID NO:348.

In an embodiment, an anti-EPCAM scFv domain comprises the sequence shown in SEQ ID NO:349, or conservative amino acid substitutions thereof. In an embodiment, an anti-EPCAM scFv domain comprises the scFv antibody 17-C20 scFv, or conservative amino acid substitutions thereof. In an embodiment, an anti-EPCAM scFv domain comprises a scFv domain that is at least 99% identical to the sequence shown in SEQ ID NO:349. In an embodiment, an anti-EPCAM scFv domain comprises a scFv domain that is at least 98% identical to the sequence shown in SEQ ID NO:349. In an embodiment, an anti-EPCAM scFv domain comprises a scFv domain that is at least 97% identical to the sequence shown in SEQ ID NO:349. In an embodiment, an anti-EPCAM scFv domain comprises a scFv domain that is at least 96% identical to the sequence shown in SEQ ID NO:349. In an embodiment, an anti-EPCAM scFv domain comprises a scFv domain that is at least 95% identical to the sequence shown in SEQ ID NO:349. In an embodiment, an anti-EPCAM scFv domain comprises a scFv domain that is at least 90% identical to the sequence shown in SEQ ID NO:349. In an embodiment, an anti-EPCAM scFv domain comprises a scFv domain that is at least 85% identical to the sequence shown in SEQ ID NO:349. In an embodiment, an anti-EPCAM scFv domain comprises a scFv domain that is at least 80% identical to the sequence shown in SEQ ID NO:349.

In an embodiment, an anti-EPCAM scFv domain comprises the sequence shown in SEQ ID NO:350, or conservative amino acid substitutions thereof. In an embodiment, an anti-EPCAM scFv domain comprises the scFv antibody 24-G6 scFv, or conservative amino acid substitutions thereof. In an embodiment, an anti-EPCAM scFv domain comprises a scFv domain that is at least 99% identical to the sequence shown in SEQ ID NO:350. In an embodiment, an anti-EPCAM scFv domain comprises a scFv domain that is at least 98% identical to the sequence shown in SEQ ID NO:350. In an embodiment, an anti-EPCAM scFv domain comprises a scFv domain that is at least 97% identical to the sequence shown in SEQ ID NO:350. In an embodiment, an anti-EPCAM scFv domain comprises a scFv domain that is at least 96% identical to the sequence shown in SEQ ID NO:350. In an embodiment, an anti-EPCAM scFv domain comprises a scFv domain that is at least 95% identical to the sequence shown in SEQ ID NO:350. In an embodiment, an anti-EPCAM scFv domain comprises a scFv domain that is at least 90% identical to the sequence shown in SEQ ID NO:350. In an embodiment, an anti-EPCAM scFv domain comprises a scFv domain that is at least 85% identical to the sequence shown in SEQ ID NO:350. In an embodiment, an anti-EPCAM scFv domain comprises a scFv domain that is at least 80% identical to the sequence shown in SEQ ID NO:350.

In an embodiment, an anti-EPCAM scFv domain comprises a V_{H} domain and a V_{L} domain, wherein the V_{H} domain comprises a sequence selected from the group consisting of SEQ ID NO:351 and fragments, derivatives, variants, and conservative amino acid substitutions thereof, and the light chain variable region (V_{L}) comprises a sequence selected from the group consisting of SEQ ID NO:352, SEQ ID NO:353, SEQ ID NO:354, SEQ ID NO:355, SEQ ID NO:356, SEQ ID NO:357, and fragments, derivatives, variants, and conservative amino acid substitutions thereof. In an embodiment, an anti-EPCAM scFv domain comprises a V_{H} region that is 99% identical to a sequence selected from the group consisting of SEQ ID NO:351 and fragments, derivatives, and variants thereof, and a V_{L} region that is at least 99% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof. In an embodiment, an anti-EPCAM scFv domain comprises a V_{H} region that is 98% identical to a sequence selected from the group consisting of SEQ ID NO:351 and fragments, derivatives, and variants thereof, and a V_{L} region that is at least 98% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof. In an embodiment, an anti-EPCAM scFv domain comprises a V_{H} region that is 97% identical to a sequence selected from the group consisting of SEQ ID NO:351 and fragments, derivatives, and variants thereof, and a V_{L} region that is at least 98% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof. In an embodiment, an anti-EPCAM scFv domain comprises a V_{H} region that is 96% identical to a sequence selected from the group consisting of SEQ ID NO:351 and fragments, derivatives, and variants thereof, and a V_{L} region that is at least 96% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof. In an embodiment, an anti-EPCAM scFv domain comprises a V_{H} region that is 95% identical to a sequence selected from the group consisting of SEQ ID NO:351 and fragments, derivatives, and variants thereof, and a V_{L} region that is at least 95% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof. In an embodiment, an anti-EPCAM scFv domain comprises a V_{H} region that is 90% identical to a sequence selected from the group consisting of SEQ ID NO:351 and fragments, derivatives, and variants thereof, and a V_{L} region that is at least 90% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof. In an embodiment, an anti-EPCAM scFv domain comprises a V_{H} region that is 85% identical to a sequence selected from the group consisting of SEQ ID NO:351 and fragments, derivatives, and variants thereof, and a V_{L} region that is at least 85% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof. In an embodiment, an anti-EPCAM scFv domain comprises a V_{H} region that is 80% identical to a sequence selected from the group consisting of SEQ ID NO:351 and fragments, derivatives, and variants thereof, and a V_{L} region that is at least 80% identical to a sequence selected from the group consisting of SEQ ID NO:352, SEQ ID NO:353, SEQ ID NO:354, SEQ ID NO:355, SEQ ID NO:356, SEQ ID NO:357, and fragments, derivatives, and variants thereof.

In an embodiment, an anti-EPCAM scFv domain comprises the heavy chain CDR1, CDR2 and CDR3 domains having the sequences set forth in SEQ ID NO:358, SEQ ID NO:359, and/or SEQ ID NO:360, respectively, or conservative amino acid substitutions thereof, and/or light chain CDR1, CDR2 and CDR3 domains having the sequences set forth in SEQ ID NO:361, SEQ ID NO:362, and/or SEQ ID NO:363, respectively, or conservative amino acid substitutions thereof.

The nucleotide sequences encoding exemplary EPCAM binding scFv domains for antibodies 3-17I scFv, 7-F17 scFv, 12-C15 scFv, 16-G5 scFv, 17-C20 scFv, and 24-G6 scFv, and fragments, variants, and derivatives thereof, as well as nucleotide sequences encoding V_{H} and V_{L} domains for additional scFv domains, are provided in Table 46 and are further described in U.S. Pat. No. 8,637,017, the disclosures of which are incorporated by reference herein. In an embodiment, a nucleotide sequence in Table 46 is codon-optimized to improve protein expression.

TABLE 46
Nucleotide sequences of exemplary EPCAM binding scFv domains.

| Identifier | Sequence (One-Letter Nucleotide Symbols) | |
|---|---|---|
| SEQ ID NO: 364 | CAGGTCCAGC TGGTGCAGTC TGGGGCTGAG GTGAAGAAGC CTGGGTCCTC GGTGAAGGTC | 60 |
| Anti-EPCAM 3-17I | TCCTGCAAGG CTTCTGGAGG CACCTTCAGC AGCTATGCTA TCAGCTGGGT GCGACAGGCC | 120 |
| scFv | CCTGGACAAG GGCTTGAGTG GATGGGAGGG ATCATCCCTA TCTETGGTAC AGCAAACTAC | 180 |
| | GCACAGAAGT TCCAGGGCAG AGTCACGATT ACCGCGGACG AATCCACGAG CACAGCCTAC | 240 |
| | ATGGAGCTGA GCAGCCTGAG ATCTGAGGAC ACGGCCGTGT ATTACTGTGC GAGAGGCCTT | 300 |
| | CTATGGAACT ACTGGGGCCA GGGAACCCTG GTCACCGTCT CCTCAAAGCT TTCAGGGAGT | 360 |
| | GCATCCGCCC CAAAACTTGA AGAAGGTGAA TTTTCAGAAG CACGCGTAGA AATTGTAATG | 420 |
| | ACACAGTCTC CAGCCACCCT GTCTGTGTCT CCAGGGGAAA GAGCCACCCT CTCCTGCAGG | 480 |
| | GCCAGTCAGA GTGTTAGCAG CAACTTAGCC TGGTACCAGC AGAAACCTGG CCAGGCTCCC | 540 |
| | AGGCTCATCA TCTATGGTGC ATCCACCACG GCCTCTGGTA TCCCAGCCAG GTTCAGTGCC | 600 |
| | AGTGGGTCTG GGACAGACTT CACTCTCACC ATCAGCAGCC TGCAGTCTGA AGATTTTGCA | 660 |
| | GTTTATTACT GTCAGCAGTA TAATAACTGG CCTCCGGCGT ACACTTTTGG CCAGGGGACC | 720 |
| | AAGCTGGAGA TCAAA | 735 |
| SEQ ID NO: 365 | CAGGTCCAGC TGGTGCAGTC TGGGGCTGAG GTGAAGAAGC CTGGGTCCTC GGTGAAGGTC | 60 |
| Anti-EPCAM 7-F17 | TCCTGCAAGG CTTCTGGAGG CACCTTCAGC AGCTATGCTA TCAGCTGGGT GCGACAGGCC | 120 |
| scFv | CCTGGACAAG GGCTTGAGTG GATGGGAGGG ATCATCCCTA TCTTTGGTAC AGCAAACTAC | 180 |
| | GCACAGAAGT TCCAGGGCAG AGTCACGATT ACCGCGGACG AATCCACGAG CACAGCCTAC | 240 |
| | ATGGAGCTGA GCAGCCTGAG ATCTGAGGAC ACGGCCGTGT ATTACTGTGC GAGAGGCCTT | 300 |
| | CTATGGAACT ACTGGGGCCA GGGAACCCTG GTCACCGTCT CCTCAAAGCT TTCAGGGAGT | 360 |
| | GCATCCGCCC CAAAACTTGA AGAAGGTGAA TTTTCAGAAG CACGCGTAGA AACGACACTC | 420 |
| | ACGCAGTCTC CAGCCACCCT GTCTGTGTCT CCAGGGGAAA GAGCCACCCT CTCCTGCAGG | 480 |
| | GCCAGTCAGA GTGTTAGCAG CAACTTAGCC TGGTACCAGC AGAAACCTGG CCAGGCTCCC | 540 |
| | AGGCTCCTCA TCTATGGTGC ATCCACCAGG GCCACTGGTA TCCCAGCCAG GTTCAGTGGC | 600 |
| | AGTGGGTCTG GGACAGAGTT CACTCTCACC ATCAGCAGCC TGCAGTCTGA AGATTTTGCA | 660 |
| | GTTTATTACT GTCAGCAGTA TAATAACTGG CCTCCGGGGT TCACTTTCGG CCCTGGGACC | 720 |
| | AAAGTGGATA TCAAA | 735 |
| SEQ ID NO: 366 | CAGGTCCAGC TGGTGCAGTC TGGGGCTGAG GTGAAGAAGC CTGGGTCCTC GGTGAAGGTC | 60 |
| Anti-EPCAM 12- | TCCTGCAAGG CTTCTGGAGG CACCTTCAGC AGCTATGCTA TCAGCTGGGT GCGACAGGCC | 120 |
| C15 scFv | CCTGGACAAG GGCTTGAGTG GATGGGAGGG ATCATCCCTA TCTETGGTAC AGCAAACTAC | 180 |
| | GCACAGAAGT TCCAGGGCAG AGTCACGATT ACCGCGGACG AATCCACGAG CACAGCCTAC | 240 |
| | ATGGAGCTGA GCAGCCTGAG ATCTGAGGAC ACGGCCGTGT ATTACTGTGC GAGAGGCCTT | 300 |
| | CTATGGAACT ACTGGGGCCA GGGAACCCTG GTCACCGTCT CCTCAAAGCT TTCAGGGAGT | 360 |
| | GCATCCGCCC CAAAACTTGA AGAAGGTGAA TTTTCAGAAG CACGCGTAGA AACGACACTC | 420 |
| | ACGCAGTCTC CAGCCACCCT GTCTTTGTCT CCAGGGGAAA GAGCCACCCT CTCCTGCAGG | 480 |
| | GCCAGTCAGA GTGTTAGCAG CAACTTAGCC TGGTACCAGC AGAAACCTGG CCAGGCTCCC | 540 |
| | AGGCTCCTCA TCTATGGTGC ATCCACCAGG GCCACTGGTA TCCCAGCCAG GTTCAGTGGC | 600 |
| | AGTGGGTCTG GGACAGAGTT CACTCTCACC ATCAGCAGCC TGCAGTCTGA AGATTTTGCA | 660 |
| | GTTTATTACT GTCAGCACTA TAATGACTGG CCTCCCACGT GGACGTTCGG CCAAGGGACC | 720 |
| | AAGCTGGAGA TCAAA | 735 |
| SEQ ID NO:367 | CAGGTCCAGC TGGTGCAGTC TGGGGCTGAG GTGAAGAAGC CTGGGTCCTC GGTGAAGGTC | 60 |
| Anti-EPCAM 16-G5 | TCCTGCAAGG CTTCTGGAGG CACCTTCAGC AGCTATGCTA TCAGCTGGGT GCGACAGGCC | 120 |
| scFv | CCTGGACAAG GGCTTGAGTG GATGGGAGGG ATCATCCCTA TCTTTGGTAC AGCAAACTAC | 180 |
| | GCACAGAAGT TCCAGGGCAG AGTCACGATT ACCGCGGACG AATCCACGAG CACAGCCTAC | 240 |
| | ATGGAGCTGA GCAGCCTGAG ATCTGAGGAC ACGGCCGTGT ATTACTGTGC GAGAGGCCTT | 300 |
| | CTATGGAACT ACTGGGGCCA GGGAACCCTG GTCACCGTCT CCTCAAAGCT TTCAGGGAGT | 360 |
| | GCATCCGCCC CAAAACTTGA AGAAGGTGAA TTTTCAGAAG CACGCGTAGA TATTGTGATG | 420 |
| | ACTCAGACTC CAGCCACCCT GTCTGTGTCT CCAGGGGAAA GAGCCACCCT CTCCTGCAGG | 480 |
| | GCCAGTCAGA GTGTTAGCAG CAACTTAGCC TGGTACCAGC AGAAACCTGG CCAGGCTCCC | 540 |
| | AGGCTCCTCA TCTATGGTGC ATCCACCAGG GCCACTGGTA TCCCAGCCAG GTTCAGTGGC | 600 |
| | AGTGGGTCTG GGACAGAGTT CACTCTCACC ATCAGCAGCC TGCAGTCTGA AGATTTTGCA | 660 |
| | GTTTATTACT GTCAGCAGTA TAATAACTGG CCTCCGTCGT GGACGTTCGG CCAAGGGACC | 720 |
| | AAGGTGGAGA TCAAA | 735 |
| SEQ ID NO: 368 | CAGGTCCAGC TGGTGCAGTC TGGGGCTGAG GTGAAGAAGC CTGGGTCCTC GGTGAAGGTC | 60 |
| Anti-EPCAM 17- | TCCTGCAAGG CTTCTGGAGG CACCTTCAGC AGCTATGCTA TCAGCTGGGT GCGACAGGCC | 120 |
| C20 scFv | CCTGGACAAG GGCTTGAGTG GATGGGAGGG ATCATCCCTA TCTETGGTAC AGCAAACTAC | 180 |
| | GCACAGAAGT TCCAGGGCAG AGTCACGATT ACCGCGGACG AATCCACGAG CACAGCCTAC | 240 |
| | ATGGAGCTGA GCAGCCTGAG ATCTGAGGAC ACGGCCGTGT ATTACTGTGC GAGAGGCCTT | 300 |
| | CTATGGAACT ACTGGGGCCA GGGAACCCTG GTCACCGTCT CCTCAAAGCT TTCAGGGAGT | 360 |
| | GCATCCGCCC CAAAACTTGA AGAAGGTGAA TTTTCAGAAG CACGCGTAGA AACGACACTC | 420 |
| | ACGCAGTCTC CAGCCACCCT GTCTGTGTCT CCAGGGGAAA GAGCCACCCT CTCCTGCAGG | 480 |
| | GCCAGTCAGA GTGTTAGCAG CAACTTAGCC TGGTACCAGC AGAAACCTGG CCAGGCTCCC | 540 |
| | AGGCTCCTCA TCTATGGTGC ATCCACCAGG GCCACTGGTA TCCCAGCCAG GTTCAGTGGC | 600 |
| | AGTGGGTCTG GGACAGAGTT CACTCTCACC ATCAGCAGCC TGCAGTCTGA AGATTTTGCA | 660 |
| | GTTTATTACT GTCAGCAGTA TAATAACTGG CCTCCGATGT ACACTTTTGG CCAGGGGACC | 720 |
| | AAGGTGGAGA TCAAA | 735 |
| SEQ ID NO: 369 | CAGGTCCAGC TGGTGCAGTC TGGGGCTGAG GTGAAGAAGC CTGGGTCCTC GGTGAAGGTC | 60 |
| Anti-EPCAM 24-G6 | TCCTGCAAGG CTTCTGGAGG CACCTTCAGC AGCTATGCTA TCAGCTGGGT GCGACAGGCC | 120 |
| scFv | CCTGGACAAG GGCTTGAGTG GATGGGAGGG ATCATCCCTA TCTTTGGTAC AGCAAACTAC | 180 |
| | GCACAGAAGT TCCAGGGCAG AGTCACGATT ACCGCGGACG AATCCACGAG CACAGCCTAC | 240 |
| | ATGGAGCTGA GCAGCCTGAG ATCTGAGGAC ACGGCCGTGT ATTACTGTGC GAGAGGCCTT | 300 |
| | CTATGGAACT ACTGGGGCCA GGGAACCCTG GTCACCGTCT CCTCAAAGCT TTCAGGGAGT | 360 |
| | GCATCCGCCC CAAAACTTGA AGAAGGTGAA TTTTCAGAAG CACGCGTAGA AACGACACTC | 420 |
| | ACGCAGTCTC CAGCCACCCT GTCTGTGTCT CCAGGGGAAA GAGCCACCCT CTCCTGCAGG | 480 |
| | GCCAGTCAGA GTGTTAGCAG CAACTTAGCC TGGTACCAGC AGAAACCTGG CCAGGCTCCC | 540 |
| | AGGCTCCTCA TCTATGGTGC ATCCACCAGG GCCACTGGTA TCCCAGCCAG GTTCAGTGGC | 600 |
| | AGTGGGTCTG GGACAGAGTT CACTCTCACC ATCAGCAGCC TGCAGTCTGA AGATTTTGCA | 660 |
| | GTTTATTACT GTCAGAAGTA TAATAACTGG CCTCCGGCCT TCACTTTCGG CCCTGGGACC | 720 |
| | AAAGTGGATA TCAAA | 735 |
| SEQ ID NO: 370 | CAGGTCCAGC TGGTGCAGTC TGGGGCTGAG GTGAAGAAGC CTGGGTCCTC GGTGAAGGTC | 60 |
| Anti-EPCAM | TCCTGCAAGG CTTCTGGAGG CACCTTCAGC AGCTATGCTA TCAGCTGGGT GCGACAGGCC | 120 |
| variable heavy | CCTGGACAAG GGCTTGAGTG GATGGGAGGG ATCATCCCTA TCTTTGGTAC AGCAAACTAC | 180 |
| chain | GCACAGAAGT TCCAGGGCAG AGTCACGATT ACCGCGGACG AATCCACGAG CACAGCCTAC | 240 |
| | ATGGAGCTGA GCAGCCTGAG ATCTGAGGAC ACGGCCGTGT ATTACTGTGC GAGAGGCCTT | 300 |
| | CTATGGAACT ACTGGGGCCA GGGAACCCTG GTCACCGTCT CCTCA | 345 |
| SEQ ID NO: 371 | GAAATTGTAA TGACACAGTC TCCAGCCACC CTGTCTGTGT CTCCAGGGGA AAGAGCCACC | 60 |
| Anti-EPCAM | CTCTCCTGCA GGGCCAGTCA GAGTGTTAGC AGCAACTTAG CCTGGTACCA GCAGAAACCT | 120 |
| variable light | GGCCAGGCTC CCAGGCTCAT CATCTATGGT GCATCCACCA CGGCCTCTGG TATCCCAGCC | 180 |
| chain | AGGTTCAGTG CCAGTGGGTC TGGGACAGAC TTCACTCTCA CCASCAGCAG CCTGCAGTCT | 240 |
| | GAAGATTTTG CAGTTTATTA CTGTCAGCAG TATAATAACT GGCCTCCGGC GTACACTTTT | 300 |
| | GGCCAGGGGA CCAAGCTGGA GATCAAA | 327 |
| SEQ ID NO: 372 | GAAACGACAC TCACGCAGTC TCCAGCCACC CTGTCTGTGT CTCCAGGGGA AAGAGCCACC | 60 |
| Anti-EPCAM | CTCTCCTGCA GGGCCAGTCA GAGTGTTAGC AGCAACTTAG CCTGGTACCA GCAGAAACCT | 120 |
| variable light | GGCCAGGCTC CCAGGCTCCT CATCTATGGT GCATCCACCA GGGCCACTGG TATCCCAGCC | 180 |
| chain | AGGTTCAGTG GCAGTGGGTC TGGGACAGAG TTCACTCTCA CCASCAGCAG CCTGCAGTCT | 240 |
| | GAAGATTTTG CAGTTTATTA CTGTCAGCAG TATAATAACT GGCCTCCGGG GTTCACTTTC | 300 |
| | GGCCCTGGGA CCAAAGTGGA TATCAAA | 327 |
| SEQ ID NO: 373 | GAAACGACAC TCACGCAGTC TCCAGCCACC CTGTCTTTGT CTCCAGGGGA AAGAGCCACC | 60 |
| Anti-EPCAM | CTCTCCTGCA GGGCCAGTCA GAGTGTTAGC AGCAACTTAG CCTGGTACCA GCAGAAACCT | 120 |
| variable light | GGCCAGGCTC CCAGGCTCCT CATCTATGGT GCATCCACCA GGGCCACTGG TATCCCAGCC | 180 |
| chain | AGGTTCAGTG GCAGTGGGTC TGGGACAGAG TTCACTCTCA CCASCAGCAG CCTGCAGTCT | 240 |
| | GAAGATTTTG CAGTTTATTA CTGTCAGCAC TATAATGACT GGCCTCCCAC GTGGACGTTC | 300 |
| | GGCCAAGGGA CCAAGCTGGA GATCAAA | 327 |
| SEQ ID NO: 374 | GATATTGTGA TGACTCAGAC TCCAGCCACC CTGTCTGTGT CTCCAGGGGA AAGAGCCACC | 60 |
| Anti-EPCAM | CTCTCCTGCA GGGCCAGTCA GAGTGTTAGC AGCAACTTAG CCTGGTACCA GCAGAAACCT | 120 |
| variable light | GGCCAGGCTC CCAGGCTCCT CATCTATGGT GCATCCACCA GGGCCACTGG TATCCCAGCC | 180 |
| chain | AGGTTCAGTG GCAGTGGGTC TGGGACAGAG TTCACTCTCA CCATCAGCAG CCTGCAGTCT | 240 |
| | GAAGATTTTG CAGTTTATTA CTGTCAGCAG TATAATAACT GGCCTCCGTC GTGGACGTTC | 300 |
| | GGCCAAGGGA CCAAGGTGGA GATCAAA | 327 |
| SEQ ID NO: 375 | GAAACGACAC TCACGCAGTC TCCAGCCACC CTGTCTGTGT CTCCAGGGGA AAGAGCCACC | 60 |
| Anti-EPCAM | CTCTCCTGCA GGGCCAGTCA GAGTGTTAGC AGCAACTTAG CCTGGTACCA GCAGAAACCT | 120 |
| variable light | GGCCAGGCTC CCAGGCTCCT CATCTATGGT GCATCCACCA GGGCCACTGG TATCCCAGCC | 180 |
| chain | AGGTTCAGTG GCAGTGGGTC TGGGACAGAG TTCACTCTCA CCASCAGCAG CCTGCAGTCT | 240 |
| | GAAGATTTTG CAGTTTATTA CTGTCAGCAG TATAATAACT GGCCTCCGAT GTACACTTTT | 300 |
| | GGCCAGGGGA CCAAGGTGGA GATCAAA | 327 |
| SEQ ID NO: 376 | GAAACGACAC TCACGCAGTC TCCAGCCACC CTGTCTGTGT CTCCAGGGGA AAGAGCCACC | 60 |
| Anti-EPCAM | CTCTCCTGCA GGGCCAGTCA GAGTGTTAGC AGCAACTTAG CCTGGTACCA GCAGAAACCT | 120 |
| variable light | GGCCAGGCTC CCAGGCTCCT CATCTATGGT GCATCCACCA GGGCCACTGG TATCCCAGCC | 180 |
| chain | AGGTTCAGTG GCAGTGGGTC TGGGACAGAG TTCACTCTCA CCASCAGCAG CCTGCAGTCT | 240 |
| | GAAGATTTTG CAGTTTATTA CTGTCAGAAG TATAATAACT GGCCTCCGGC CTTCACTTTC | 300 |
| | GGCCCTGGGA CCAAAGTGGA TATCAAA | 327 |

In an embodiment, an anti-EPCAM scFv domain comprises the scFv antibody 3-17I scFv encoded by a nucleotide sequence. In an embodiment, an anti-EPCAM scFv domain is encoded by the sequence shown in SEQ ID NO:364. In an embodiment, an anti-EPCAM scFv domain is encoded by a nucleotide sequence that is at least 99% identical to the sequence shown in SEQ ID NO:364. In an embodiment, an anti-EPCAM scFv domain is encoded by a nucleotide sequence that is at least 98% identical to the sequence shown in SEQ ID NO:364. In an embodiment, an anti-EPCAM scFv domain is encoded by a nucleotide sequence that is at least 97% identical to the sequence shown in SEQ ID NO:364. In an embodiment, an anti-EPCAM scFv domain is encoded by a nucleotide sequence that is at least 96% identical to the sequence shown in SEQ ID NO:364. In an embodiment, an anti-EPCAM scFv domain is encoded by a nucleotide sequence that is at least 95% identical to the sequence shown in SEQ ID NO:364. In an embodiment, an anti-EPCAM scFv domain is encoded by a nucleotide sequence that is at least 90% identical to the sequence shown in SEQ ID NO:364. In an embodiment, an anti-EPCAM scFv domain is encoded by a nucleotide sequence that is at least 85% identical to the sequence shown in SEQ ID NO:364. In an embodiment, an anti-EPCAM scFv domain is encoded by a nucleotide sequence that is at least 80% identical to the sequence shown in SEQ ID NO:364. In an embodiment, including the foregoing embodiments, SEQ ID NO:364 is codon-optimized to improve protein expression.

In an embodiment, an anti-EPCAM scFv domain comprises the scFv antibody 7-F17 scFv encoded by a nucleotide sequence. In an embodiment, an anti-EPCAM scFv domain is encoded by the sequence shown in SEQ ID NO:365. In an embodiment, an anti-EPCAM scFv domain is encoded by a nucleotide sequence that is at least 99% identical to the sequence shown in SEQ ID NO:365. In an embodiment, an anti-EPCAM scFv domain is encoded by a nucleotide sequence that is at least 98% identical to the sequence shown in SEQ ID NO:365. In an embodiment, an anti-EPCAM scFv domain is encoded by a nucleotide sequence that is at least 97% identical to the sequence shown in SEQ ID NO:365. In an embodiment, an anti-EPCAM scFv domain is encoded by a nucleotide sequence that is at least 96% identical to the sequence shown in SEQ ID NO:365. In an embodiment, an anti-EPCAM scFv domain is encoded by a nucleotide sequence that is at least 95% identical to the sequence shown in SEQ ID NO:365. In an embodiment, an anti-EPCAM scFv domain is encoded by a nucleotide sequence that is at least 90% identical to the sequence shown in SEQ ID NO:365. In an embodiment, an anti-EPCAM scFv domain is encoded by a nucleotide sequence that is at least 85% identical to the sequence shown in SEQ ID NO:365. In an embodiment, an anti-EPCAM scFv domain is encoded by a nucleotide sequence that is at least 80% identical to the sequence shown in SEQ ID NO:365. In an embodiment, including the foregoing embodiments, SEQ ID NO:365 is codon-optimized to improve protein expression.

In an embodiment, an anti-EPCAM scFv domain comprises the scFv antibody 12-C15 scFv encoded by a nucleotide sequence. In an embodiment, an anti-EPCAM scFv domain is encoded by the sequence shown in SEQ ID NO:366. In an embodiment, an anti-EPCAM scFv domain is encoded by a nucleotide sequence that is at least 99% identical to the sequence shown in SEQ ID NO:366. In an embodiment, an anti-EPCAM scFv domain is encoded by a nucleotide sequence that is at least 98% identical to the sequence shown in SEQ ID NO:366. In an embodiment, an anti-EPCAM scFv domain is encoded by a nucleotide sequence that is at least 97% identical to the sequence shown in SEQ ID NO:366. In an embodiment, an anti-EPCAM scFv domain is encoded by a nucleotide sequence that is at least 96% identical to the sequence shown in SEQ ID NO:366. In an embodiment, an anti-EPCAM scFv domain is encoded by a nucleotide sequence that is at least 95% identical to the sequence shown in SEQ ID NO:366. In an embodiment, an anti-EPCAM scFv domain is encoded by a nucleotide sequence that is at least 90% identical to the sequence shown in SEQ ID NO:366. In an embodiment, an anti-EPCAM scFv domain is encoded by a nucleotide sequence that is at least 85% identical to the sequence shown in SEQ ID NO:366. In an embodiment, an anti-EPCAM scFv domain is encoded by a nucleotide sequence that is at least 80% identical to the sequence shown in SEQ ID NO:366. In an embodiment, including the foregoing embodiments, SEQ ID NO:366 is codon-optimized to improve protein expression.

In an embodiment, an anti-EPCAM scFv domain comprises the scFv antibody 16-G5 scFv encoded by a nucleotide sequence. In an embodiment, an anti-EPCAM scFv domain is encoded by the sequence shown in SEQ ID NO:367. In an embodiment, an anti-EPCAM scFv domain is encoded by a nucleotide sequence that is at least 99% identical to the sequence shown in SEQ ID NO:367. In an embodiment, an anti-EPCAM scFv domain is encoded by a nucleotide sequence that is at least 98% identical to the sequence shown in SEQ ID NO:367. In an embodiment, an anti-EPCAM scFv domain is encoded by a nucleotide sequence that is at least 97% identical to the sequence shown in SEQ ID NO:367. In an embodiment, an anti-EPCAM scFv domain is encoded by a nucleotide sequence that is at least 96% identical to the sequence shown in SEQ ID NO:367. In an embodiment, an anti-EPCAM scFv domain is encoded by a nucleotide sequence that is at least 95% identical to the sequence shown in SEQ ID NO:367. In an embodiment, an anti-EPCAM scFv domain is encoded by a nucleotide sequence that is at least 90% identical to the sequence shown in SEQ ID NO:367. In an embodiment, an anti-EPCAM scFv domain is encoded by a nucleotide sequence that is at least 85% identical to the sequence shown in SEQ ID NO:367. In an embodiment, an anti-EPCAM scFv domain is encoded by a nucleotide sequence that is at least 80% identical to the sequence shown in SEQ ID NO:367. In an embodiment, including the foregoing embodiments, SEQ ID NO:367 is codon-optimized to improve protein expression.

In an embodiment, an anti-EPCAM scFv domain comprises the scFv antibody 17-C20 scFv encoded by a nucleotide sequence. In an embodiment, an anti-EPCAM scFv domain is encoded by the sequence shown in SEQ ID NO:368. In an embodiment, an anti-EPCAM scFv domain is encoded by a nucleotide sequence that is at least 99% identical to the sequence shown in SEQ ID NO:368. In an embodiment, an anti-EPCAM scFv domain is encoded by a nucleotide sequence that is at least 98% identical to the sequence shown in SEQ ID NO:368. In an embodiment, an anti-EPCAM scFv domain is encoded by a nucleotide sequence that is at least 97% identical to the sequence shown in SEQ ID NO:368. In an embodiment, an anti-EPCAM scFv domain is encoded by a nucleotide sequence that is at least 96% identical to the sequence shown in SEQ ID NO:368. In an embodiment, an anti-EPCAM scFv domain is encoded by a nucleotide sequence that is at least 95% identical to the sequence shown in SEQ ID NO:368. In an embodiment, an anti-EPCAM scFv domain is encoded by a nucleotide sequence that is at least 90% identical to the sequence shown in SEQ ID NO:368. In an embodiment, an anti-EPCAM scFv domain is encoded by a nucleotide sequence that is at least 85% identical to the sequence shown in SEQ ID NO:368. In an embodiment, an anti-EPCAM scFv domain is encoded by a nucleotide sequence that is at least 80% identical to the sequence shown in SEQ ID NO:368. In an embodiment, including the foregoing embodiments, SEQ ID NO:368 is codon-optimized to improve protein expression.

In an embodiment, an anti-EPCAM scFv domain comprises the scFv antibody 24-G6 scFv encoded by a nucleotide sequence. In an embodiment, an anti-EPCAM scFv domain is encoded by the sequence shown in SEQ ID NO:369. In an embodiment, an anti-EPCAM scFv domain is encoded by a nucleotide sequence that is at least 99% identical to the sequence shown in SEQ ID NO:369. In an embodiment, an anti-EPCAM scFv domain is encoded by a nucleotide sequence that is at least 98% identical to the sequence shown in SEQ ID NO:369. In an embodiment, an anti-EPCAM scFv domain is encoded by a nucleotide sequence that is at least 97% identical to the sequence shown in SEQ ID NO:369. In an embodiment, an anti-EPCAM scFv domain is encoded by a nucleotide sequence that is at least 96% identical to the sequence shown in SEQ ID NO:369. In an embodiment, an anti-EPCAM scFv domain is encoded by a nucleotide sequence that is at least 95% identical to the sequence shown in SEQ ID NO:369. In an embodiment, an anti-EPCAM scFv domain is encoded by a nucleotide sequence that is at least 90% identical to the sequence shown in SEQ ID NO:369. In an embodiment, an anti-EPCAM scFv domain is encoded by a nucleotide sequence that is at least 85% identical to the sequence shown in SEQ ID NO:369. In an embodiment, an anti-EPCAM scFv domain is encoded by a nucleotide sequence that is at least 80% identical to the sequence shown in SEQ ID NO:369. In an embodiment, including the foregoing embodiments, SEQ ID NO:369 is codon-optimized to improve protein expression.

In an embodiment, an anti-EPCAM scFv domain comprises a V_{H} domain and a V_{L} domain, wherein the V_{H} domain is encoded by a sequence selected from the group consisting of SEQ ID NO:370 and fragments, derivatives, variants, and conservative amino acid substitutions thereof, and the light chain variable region (V_{L}) comprises a sequence selected from the group consisting of SEQ ID NO:371, SEQ ID NO:372, SEQ ID NO:372, SEQ ID NO:374, SEQ ID NO:375, SEQ ID NO:376, and fragments, derivatives, variants, and conservative amino acid substitutions thereof. In an embodiment, an anti-EPCAM scFv domain comprises a V_{H} region that is 99% identical to a sequence selected from the group consisting of SEQ ID NO:370 and fragments, derivatives, and variants thereof, and a V_{L} region that is at least 99% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof. In an embodiment, an anti-EPCAM scFv domain comprises a V_{H} region that is 98% identical to a sequence selected from the group consisting of SEQ ID NO:370 and fragments, derivatives, and variants thereof, and a V_{L} region that is at least 98% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof. In an embodiment, an anti-EPCAM scFv domain comprises a V_{H} region that is 97% identical to a sequence selected from the group consisting of SEQ ID NO:370 and fragments, derivatives, and variants thereof, and a V_{L} region that is at least 97% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof. In an embodiment, an anti-EPCAM scFv domain comprises a V_{H} region that is 96% identical to a sequence selected from the group consisting of SEQ ID NO:370 and fragments, derivatives, and variants thereof, and a V_{L} region that is at least 96% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof. In an embodiment, an anti-EPCAM scFv domain comprises a V_{H} region that is 95% identical to a sequence selected from the group consisting of SEQ ID NO:370 and fragments, derivatives, and variants thereof, and a V_{L} region that is at least 95% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof. In an embodiment, an anti-EPCAM scFv domain comprises a V_{H} region that is 90% identical to a sequence selected from the group consisting of SEQ ID NO:370 and fragments, derivatives, and variants thereof, and a V_{L} region that is at least 90% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof. In an embodiment, an anti-EPCAM scFv domain comprises a V_{H} region that is 85% identical to a sequence selected from the group consisting of SEQ ID NO:370 and fragments, derivatives, and variants thereof and a V_{L} region that is at least 85% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof. In an embodiment, an anti-EPCAM scFv domain comprises a V_{H} region that is 80% identical to a sequence selected from the group consisting of SEQ ID NO:370 and fragments, derivatives, and variants thereof and a V_{L} region that is at least 80% identical to a sequence selected from the group consisting of SEQ ID NO:371, SEQ ID NO:372, SEQ ID NO:373, SEQ ID NO:374, SEQ ID NO:375, SEQ ID NO:376, and fragments, derivatives, and variants thereof.

In some embodiments, the EPCAM binding domain includes scFv, VH, VL, and CDR domains as described in U.S. Pat. No. 9,388,249, the disclosures of which are incorporated by reference herein. The amino acid sequences of exemplary EPCAM binding domains for use in the CCRs of the present invention are provided in Table 47.

TABLE 47
Amino acid sequences of exemplary extracellular EPCAM binding domains.

| Identifier | Sequence (One-Letter Amino Acid Symbols) | |
|---|---|---|
| SEQ ID NO: 377 | QVQLQESGGG LVQPGGSLRL SCTTSGFTFT SYAMSWVRQA PGKGLEWVSS ISGSGGITYY | 60 |
| Anti-EPCAM | ADSVKGRFTI SRDNSKNTLY LQMNSLRAED TAVYYCAKDR VLVPASSSYF DYWGQGTLVT | 120 |
| variable heavy | VSR | 123 |
| chain | | |
| SEQ ID NO: 378 | SSELTQDPAV SVALGQTVRI TCQGDSLRSY YASWYQQKPG QAPVLVIYGK NNRPSGIPDR | 60 |
| Anti-EPCAM | FSGSSSGNTA SLTITGAQAE DEADYYCNSR DSSGNHVVFG GGTKLTVLG | 109 |
| variable light | | |
| chain | | |
| SEQ ID NO: 379 | SYAMS | 5 |
| Anti-EPCAM CDR1 | | |
| heavy chain | | |
| SEQ ID NO: 380 | SISGSGGITY YADSVKG | 17 |
| Anti-EPCAM CDR2 | | |
| heavy chain | | |
| SEQ ID NO: 381 | DRVLVPASSS YFDY | 14 |
| Anti-EPCAM CDR3 | | |
| heavy chain | | |
| SEQ ID NO: 382 | QGDSLRSYYA S | 11 |
| Anti-EPCAM CDR1 | | |
| light chain | | |
| SEQ ID NO: 383 | GKNNRPS | 7 |
| Anti-EPCAM CDR2 | | |
| light chain | | |
| SEQ ID NO: 384 | NSRDSSGNE | 9 |
| Anti-EPCAM CDR3 | | |
| light chain | | |

In an embodiment, an anti-EPCAM scFv domain comprises a V_{H} domain and a V_{L} domain, wherein the V_{H} domain comprises the sequence shown in SEQ ID NO:377, or conservative amino acid substitutions thereof, and the light chain variable region (V_{L}) comprises the sequence shown in SEQ ID NO:378, or conservative amino acid substitutions thereof. In an embodiment, an anti-EPCAM scFv domain comprises V_{H} and V_{L} regions that are each at least 99% identical to the sequences shown in SEQ ID NO:377 and SEQ ID NO:378, respectively. In an embodiment, an anti-EPCAM scFv domain comprises V_{H} and V_{L} regions that are each at least 98% identical to the sequences shown in SEQ ID NO:377 and SEQ ID NO:378, respectively. In an embodiment, an anti-EPCAM scFv domain comprises V_{H} and V_{L} regions that are each at least 97% identical to the sequences shown in SEQ ID NO:377 and SEQ ID NO:378, respectively. In an embodiment, an anti-EPCAM scFv domain comprises V_{H} and V_{L} regions that are each at least 96% identical to the sequences shown in SEQ ID NO:377 and SEQ ID NO:378, respectively. In an embodiment, an anti-EPCAM scFv domain comprises V_{H} and V_{L} regions that are each at least 95% identical to the sequences shown in SEQ ID NO:377 and SEQ ID NO:378, respectively. In an embodiment, an anti-EPCAM scFv domain comprises V_{H} and V_{L} regions that are each at least 90% identical to the sequences shown in SEQ ID NO:377 and SEQ ID NO:378, respectively. In an embodiment, an anti-EPCAM scFv domain comprises V_{H} and V_{L} regions that are each at least 85% identical to the sequences shown in SEQ ID NO:377 and SEQ ID NO:378, respectively. In an embodiment, an anti-EPCAM scFv domain comprises V_{H} and V_{L} regions that are each at least 80% identical to the sequences shown in SEQ ID NO:377 and SEQ ID NO:378, respectively.

In an embodiment, an anti-EPCAM scFv domain comprises the heavy chain CDR1, CDR2 and CDR3 domains having the sequences set forth in SEQ ID NO:379, SEQ ID NO:380, and/or SEQ ID NO:381, respectively, or conservative amino acid substitutions thereof, and/or light chain CDR1, CDR2 and CDR3 domains having the sequences set forth in SEQ ID NO:382, SEQ ID NO:383, and/or SEQ ID NO:384, respectively, or conservative amino acid substitutions thereof.

In an embodiment, the anti-EPCAM binding domain includes additional scFv, V_{H} and/or V_{L} sequences or a heavy chain and/or a light chain CDR1, CDR2, and/or CDR3 sequence as disclosed in U.S. Pat. No. 9,388,249, the disclosures of which are incorporated by reference herein.

In an embodiment, the anti-EPCAM binding domain includes additional scFv, V_{H} and/or V_{L} sequences or a heavy chain and/or a light chain CDR1, CDR2, and/or CDR3 sequence as disclosed in U.S. Patent Application Publication No. US 2019/0233536 A1, the disclosures of which are incorporated by reference herein.

### 7. Extracellular Tissue Factor Binding Domains

In an embodiment, a CCR of the present invention comprises an extracellular domain, wherein the extracellular domain comprises a tissue factor (TF) binding domain, also referred to herein as an anti-TF domain. TF is a transmembrane glycoprotein with a 219 amino acid residue extracellular region, a 23 amino acid residue trans membrane region and a 21 amino acid residue cytoplasmic region, which initiates blood coagulation in conjunction with factor VIIa. TF is expressed in lung, pancreatic, breast, colon, and gastric carcinomas. Hu, et al., Oncol. Res. 1994, 6, 321-327; Callander, et al., Cancer 1992, 70, 1194-201. Abnormally high expression of TF has been shown clinically to be associated with poor differentiation of many tumors, including in colorectal cancer, NSCLC, and breast cancer. Shigernori, et al., Thromb. Haemost. 1998, 80, 894-898; Seto, et al., Cancer 2000, 88, 295-301; Sawada, et al., Br. J Cancer 1999, 79, 472-477; Kirschmann, et al., Breast Cancer Res. Treat. 1999, 55, 127-136; Schwirzke, et al., Anticancer Res. 1999, 19, 1801-1814. In an embodiment, the TF binding domain is an scFv domain. In an embodiment, the CCR comprises an extracellular domain that binds to human TF. In an embodiment, the extracellular domain binds to murine TF. In an embodiment, the extracellular TF binding domain is a scFv domain. In an embodiment, a CCR of the present invention comprises a construct as shown in FIG. 34, wherein the V_{H} and V_{L} domains are anti-TF V_{H} and V_{L} domains, and the linker is as described herein.

In an embodiment, the TF binding domain includes scFv antibodies prepared from the CDR, V_{H}, and V_{L} domains described in U.S. Pat. No. 7,993,644, the disclosures of which are incorporated by reference herein. In an embodiment, the scFv domain includes a scFv, V_{H}, V_{L}, or CDR domain of the antibodies TF260, TF196, TF278, TF277, TF392, or TF9, the preparation and properties of each of which are described in U.S. Pat. No. 7,993,644 and are incorporated by reference herein, including the V_{H}, V_{L}, and CDR domains of each of TF260, TF196, TF278, TF277, TF392, or TF9. In an embodiment, the scFv includes a scFv, V_{H}, V_{L}, or CDR domain of tisotumab, or variants, fragments, or derivatives thereof, the structure of which is described, along with other scFv, V_{H}, V_{L}, or CDR domains that may be used in embodiments of the present invention, in U.S. Patent Application Publication Nos. US 2019/0169311 A1, US 2019/0315880 A1, US 2020/0246477 A1, and US 2021/0030888 A1, the disclosures of which are incorporated by reference herein. In an embodiment, the scFv includes the antibodies described in U.S. Pat. No. 9,168,314, the disclosures of which are incorporated by reference herein. In an embodiment, the scFv includes the antibodies described in U.S. Pat. No. 7,824,677, the disclosures of which are incorporated by reference herein. The amino acid sequences of exemplary TF binding scFv domains are provided in Table 48.

TABLE 48
Amino acid sequences of exemplary tissue factor binding scFv domains.

| Identifier | Sequence (One-Letter Amino Acid Symbols) | |
|---|---|---|
| SEQ ID NO: 385 | QVQLKQSGAE LMKPGASVKI SCKATGYTFS SYWIEWVKQR PGHGLEWIGE ILPGSGSTNY | 60 |
| Anti-TF variable | NEKFKGKATF TADTSSNTAY MQLSSLTSED SAVYYCARED RYDGDYWGQG TTLTVS | 116 |
| heavy chain | | |
| TF260 | | |
| SEQ ID NO: 386 | QAVVTQESAL TTSPGETVTL TCRSSTGAVT TSNYANWVQE KPDHLFTGLI GGTNNRAPGV | 60 |
| Anti-TF variable | PARFSGSLIG DKAALTITGA QTEDEAIYFC ALWYSNEWVF GGGTKLTVLG QP | 112 |
| light chain | | |
| TF260 | | |
| SEQ ID NO: 387 | QVQLKQSGPE LEKPGASVKI SCKASGYSFT GYNMNWVKQS NGKSLEWIGN IDPYYGGTSY | 60 |
| Anti-TF variable | NQKFKGKATL TVDKSSNTAY MHLKSLTSED SAVYYCARDS SSWFAYWGQG TLVTVSA | 117 |
| heavy chain | | |
| TF196 | | |
| SEQ ID NO: 388 | DIQLTQSPAS LSASVGETVT ITCRASGNIH NYLAWYQQKQ GKSPQLLVYN AKTLADGVPS | 60 |
| Anti-TF variable | RFSGSGSGTQ YSLKINSLQP EDFGSYYCQH FWITPWTFGG GTKLEI | 106 |
| light chain | | |
| TF196 | | |
| SEQ ID NO: 389 | EVQLQQSGAE LMKPGASVKI SCKATGYTFS SYWIEWVKQR PGHGLEWIGE ILPGSASTKY | 60 |
| Anti-TF variable | NEKFKGKATF TADTSSNTAY MQLSSLTSED SAVYYCARDY YYGSSYGFAY WGQGTLVTVS | 120 |
| heavy chain | S | 121 |
| TF278 | | |
| SEQ ID NO: 390 | QAVVTQESAL TTSPGETVTL TCRSSTGAVT TSNYANWVQE KPDHLFTGLI GGTNNRGPGV | 60 |
| Anti-TF variable | PARFSGSLIG DKAALTITGA QTEDEAVYFC ALWYSNEWVF GGGTKLTVLG | 110 |
| light chain | | |
| TF278 | | |
| SEQ ID NO: 391 | QVQLQQPGAE LVKPGASVKL SCKTSGYTFT SYWMEWVKQR PGQGLEWIGE IDPSDSYTNY | 60 |
| Anti-TF variable | NQKFKGKATL TVDKSSSTAY MQLSSLTSED SAVYYCTYYV NYYAMDYWGQ GTSVTVSS | 118 |
| heavy chain | | |
| TF277 | | |
| SEQ ID NO: 392 | FSGSGSGTFY SLTISSVEAE DAADYYCHQW SSYPYTFGGG TKLEIK SNLASGVPSR | 60 |
| Anti-TF variable | QIVLTQSPAI MSASLGEEIT LTCSASSSVS YMHWYQQKSG TSPKLLIYST | 106 |
| light chain | | |
| TF277 | | |
| SEQ ID NO: 393 | QVQLKESGAE LMKPGASVKI SCKATGYTFS SYWIEWVKQR PGHGLEWIGE ILPGSGSTNY | 60 |
| Anti-TF variable | NEKFKGKATF TADTSSNTAY MQLSSLTSED SAVYYCARDR NGYVNYFDSW GQGTTLTVSS | 120 |
| heavy chain | | |
| TF392 | | |
| SEQ ID NO: 394 | QAVVTQESAL TTSPGETVTL TCRSSTGAVT TSNYANWVQE KPDHLFTGLI GGTNNRAPGV | 60 |
| Anti-TF variable | PARFSGSLIG DKAALTITGA QTEDEAIYFC ALWYSNEWVF GGGTKLTVLG QP | 112 |
| light chain | | |
| TF392 | | |
| SEQ ID NO: 395 | DVKLQESGPD LVKPSQSLSL TCTVTGYSIT SGYSWHWIRQ FPGNKLEWMG YIHYSGSTKY | 60 |
| Anti-TF variable | NPSLKSRISI TRDTSKNQFF LQLNSVTTED TATYYCARLW SWYFDVWGAG TTVTVSS | 117 |
| heavy chain TF9 | | |
| SEQ ID NO: 396 | NIMMTQSPSS LAVSAGEKVT MSCKSSQSVL YSSNQKNYLA WYQQKPGQSP KLLIYWASTR | 60 |
| Anti-TF variable | ESGVPDRFTG SGSGTDFTLT ISSVQAEDLA VYYCEQYLSS YTFGGGTKLE IK | 112 |
| light chain TF9 | | |
| SEQ ID NO: 397 | SYWIE | 5 |
| Anti-TF variable | | |
| heavy chain CDR1 | | |
| TF260 | | |
| SEQ ID NO: 398 | EILPGSGSTN YNEKFKG | 17 |
| Anti-TF variable | | |
| heavy chain CDR2 | | |
| TF260 | | |
| SEQ ID NO: 399 | EDRYDGDY | 8 |
| Anti-TF variable | | |
| heavy chain CDR3 | | |
| TF260 | | |
| SEQ ID NO: 400 | RSSTGAVTTS NYAN | 14 |
| Anti-TF variable | | |
| heavy chain CDR1 | | |
| TF260 | | |
| SEQ ID NO: 401 | GTNNRAP | 7 |
| Anti-TF variable | | |
| heavy chain CDR2 | | |
| TF260 | | |
| SEQ ID NO: 402 | ALWYSNEWV | 9 |
| Anti-TF variable | | |
| heavy chain CDR3 | | |
| TF260 | | |
| SEQ ID NO: 403 | GYNMN | 5 |
| Anti-TF variable | | |
| heavy chain CDR1 | | |
| TF196 | | |
| SEQ ID NO: 404 | NIDPYYGGTS YNQKFKG | 17 |
| Anti-TF variable | | |
| heavy chain CDR2 | | |
| TF196 | | |
| SEQ ID NO: 405 | DSSSWFAY | 8 |
| Anti-TF variable | | |
| heavy chain CDR3 | | |
| TF196 | | |
| SEQ ID NO: 406 | RASGNIENYL A | 11 |
| Anti-TF variable | | |
| heavy chain CDR1 | | |
| TF196 | | |
| SEQ ID NO: 407 | NAKTLAD | 7 |
| Anti-TF variable | | |
| heavy chain CDR2 | | |
| TF196 | | |
| SEQ ID NO: 408 | QHFWITPWT | 9 |
| Anti-TF variable | | |
| heavy chain CDR3 | | |
| TF196 | | |
| SEQ ID NO: 409 | SGYSWH | 6 |
| Anti-TF variable | | |
| heavy chain CDR1 | | |
| TF9 | | |
| SEQ ID NO: 410 | YIHYSGSTKY NPSLKS | 16 |
| Anti-TF variable | | |
| heavy chain CDR2 | | |
| TF9 | | |
| SEQ ID NO: 411 | LWSWYFDV | 8 |
| Anti-TF variable | | |
| heavy chain CDR3 | | |
| TF9 | | |
| SEQ ID NO: 412 | KSSQSVLYSS NQKNYLA | 17 |
| Anti-TF variable | | |
| heavy chain CDR1 | | |
| TF9 | | |
| SEQ ID NO: 413 | WASTRES | 7 |
| Anti-TF variable | | |
| heavy chain CDR2 | | |
| TF9 | | |
| SEQ ID NO: 414 | HQYLSSYT | 8 |
| Anti-TF variable | | |
| heavy chain CDR3 | | |
| TF9 | | |
| SEQ ID NO: 415 | EVQLLESGGG LVQPGGSLRL SCAASGFTFS NYAMSWVRQA PGKGLEWVSS ISGSGDYTYY | 60 |
| Anti-TF variable | TDSVKGRFTI SRDNSKNTLY LQMNSLRAED TAVYYCARSP WGYYLDSWGQ GTLVTVSS | 118 |
| heavy chain | | |
| SEQ ID NO: 416 | DIQMTQSPPS LSASAGDRVT ITCRASQGIS SRLAWYQQKP EKAPKSLIYA ASSLQSGVPS | 60 |
| Anti-TF variable | RFSGSGSGTD FTLTISSLQP EDFATYYCQQ YNSYPYTFGQ GTKLEIK | 107 |
| light chain | | |
| SEQ ID NO: 417 | QVQLVQSGAE VRKPGSSVKV SCKASGGSFN NYPIFWVRQA PGQGFEWMGR IIPILGITAY | 60 |
| Anti-TF variable | AQKFQGRVTI TADKSTSTAY MELNSLRSED TAVYYCAGGD DLDAFDIWGQ GTMVSVSS | 118 |
| heavy chain | | |
| SEQ ID NO: 418 | DIQMTQSPSS LSASVGDRVT ITCRASQGIS SWLAWYQQKP EKAPKSLIYA ASSLQSGVPS | 60 |
| Anti-TF variable | RFSGSGSGTD FTLTISSLQP EDFATYYCQQ YNSYPYTFGQ GTKLEIK | 107 |
| light chain | | |
| SEQ ID NO: 419 | QVQLVESGGG VVQPGRSLRL SCAGSGFTEN RYAMYWVRQA PGKGLDWVAV ISNDGINKYY | 60 |
| Anti-TF variable | ADSVKGRFTI SRDNSKNTLY LQMNSLRAED TAVYYCARDH TMVRGAFDYW GQGTLVTVSS | 120 |
| heavy chain | | |
| SEQ ID NO: 420 | EIVLTQSPAT LSLSPGERAT LSCRASQSVS SYLAWYQQKP GQAPRLLIYD ASNRATGIPA | 60 |
| Anti-TF variable | RFSGSGSGTD FTLTISSLEP EDFAVYYCQQ RSNWPLTFGG GTKVEIK | 107 |
| light chain | | |
| SEQ ID NO: 421 | QVQLVESGGG VVQPGRSLRL SCVASGFTVS NDGMEWVRQA PGKGLEWVAL IWYDGVNKNY | 60 |
| Anti-TF variable | ADSVKGRFTI SRDKSKNTLY LQMNSLRAED TAVYYCARRP GTFYGLDVWG QGTTVTVSS | 119 |
| heavy chain | | |
| SEQ ID NO: 422 | EIVLTQSPGT LSLSPGERAT LSCRASQSVS SSYLAWYQQK PGQAPRLLIY GASSRATGIP | 60 |
| Anti-TF variable | DRFSGSGSGT DFTLTISRLE PEDFAVYYCQ QYGSSLTFGG GTKVEIK | 107 |
| light chain | | |
| SEQ ID NO: 423 | MEIQLQQSGP ELVKPGASVQ VSCKTSGYSF THENVYWVRQ SHGKSLEWIG YIDPDNGITF | 60 |
| Anti-TF variable | YDENFMGKAT LTVDKSSTTA EMHLNSLTSD DSAVYFCARD VTTAVDFWGQ GTTLTVSS | 118 |
| heavy chain | | |
| SEQ ID NO: 424 | DIQMTQSPAS QSASLGESVT ITCLATQTLD TWLAWYQQKP GKSPQLLIYA ATYLADGVPS | 60 |
| Anti-TF variable | RFSGSGSGTK FSFKISSLQA EDFVNYYCQL VYSSPSTFGA GTKLELK | 107 |
| light chain | | |

In an embodiment, an anti-TF scFv domain comprises a heavy chain variable region (V_{H}) domain and/or a light chain variable region (V_{L}) domain of the scFv antibody TF260, or variants, derivatives, fragments, or conservative amino acid substitutions thereof. In an embodiment, an anti-TF scFv domain comprises a V_{H} domain and a V_{L} domain, wherein the V_{H} domain comprises the sequence shown in SEQ ID NO:385, or conservative amino acid substitutions thereof, and the light chain variable region (V_{L}) comprises the sequence shown in SEQ ID NO:386, or conservative amino acid substitutions thereof. In an embodiment, an anti-TF scFv domain comprises V_{H} and V_{L} regions that are each at least 99% identical to the sequences shown in SEQ ID NO:385 and SEQ ID NO:386, respectively. In an embodiment, an anti-TF scFv domain comprises V_{H} and V_{L} regions that are each at least 98% identical to the sequences shown in SEQ ID NO:385 and SEQ ID NO:386, respectively. In an embodiment, an anti-TF scFv domain comprises V_{H} and V_{L} regions that are each at least 97% identical to the sequences shown in SEQ ID NO:385 and SEQ ID NO:386, respectively. In an embodiment, an anti-TF scFv domain comprises V_{H} and V_{L} regions that are each at least 96% identical to the sequences shown in SEQ ID NO:385 and SEQ ID NO:386, respectively. In an embodiment, an anti-TF scFv domain comprises V_{H} and V_{L} regions that are each at least 95% identical to the sequences shown in SEQ ID NO:385 and SEQ ID NO:386, respectively. In an embodiment, an anti-TF scFv domain comprises V_{H} and V_{L} regions that are each at least 90% identical to the sequences shown in SEQ ID NO:385 and SEQ ID NO:386, respectively. In an embodiment, an anti-TF scFv domain comprises V_{H} and V_{L} regions that are each at least 85% identical to the sequences shown in SEQ ID NO:385 and SEQ ID NO:386, respectively. In an embodiment, an anti-TF scFv domain comprises V_{H} and V_{L} regions that are each at least 80% identical to the sequences shown in SEQ ID NO:385 and SEQ ID NO:386, respectively.

In an embodiment, an anti-TF scFv domain comprises a heavy chain variable region (V_{H}) domain and/or a light chain variable region (V_{L}) domain of the scFv antibody TF196, or variants, derivatives, fragments, or conservative amino acid substitutions thereof. In an embodiment, an anti-TF scFv domain comprises a V_{H} domain and a V_{L} domain, wherein the V_{H} domain comprises the sequence shown in SEQ ID NO:387, or conservative amino acid substitutions thereof, and the light chain variable region (V_{L}) comprises the sequence shown in SEQ ID NO:388, or conservative amino acid substitutions thereof. In an embodiment, an anti-TF scFv domain comprises V_{H} and V_{L} regions that are each at least 99% identical to the sequences shown in SEQ ID NO:387 and SEQ ID NO:388, respectively. In an embodiment, an anti-TF scFv domain comprises V_{H} and V_{L} regions that are each at least 98% identical to the sequences shown in SEQ ID NO:387 and SEQ ID NO:388, respectively. In an embodiment, an anti-TF scFv domain comprises V_{H} and V_{L} regions that are each at least 97% identical to the sequences shown in SEQ ID NO:387 and SEQ ID NO:388, respectively. In an embodiment, an anti-TF scFv domain comprises V_{H} and V_{L} regions that are each at least 96% identical to the sequences shown in SEQ ID NO:387 and SEQ ID NO:388, respectively. In an embodiment, an anti-TF scFv domain comprises V_{H} and V_{L} regions that are each at least 95% identical to the sequences shown in SEQ ID NO:387 and SEQ ID NO:388, respectively. In an embodiment, an anti-TF scFv domain comprises V_{H} and V_{L} regions that are each at least 90% identical to the sequences shown in SEQ ID NO:387 and SEQ ID NO:388, respectively. In an embodiment, an anti-TF scFv domain comprises V_{H} and V_{L} regions that are each at least 85% identical to the sequences shown in SEQ ID NO:387 and SEQ ID NO:388, respectively. In an embodiment, an anti-TF scFv domain comprises V_{H} and V_{L} regions that are each at least 80% identical to the sequences shown in SEQ ID NO:387 and SEQ ID NO:388, respectively.

In an embodiment, an anti-TF scFv domain comprises a heavy chain variable region (V_{H}) domain and/or a light chain variable region (V_{L}) domain of the scFv antibody TF278, or variants, derivatives, fragments, or conservative amino acid substitutions thereof. In an embodiment, an anti-TF scFv domain comprises a V_{H} domain and a V_{L} domain, wherein the V_{H} domain comprises the sequence shown in SEQ ID NO:389, or conservative amino acid substitutions thereof, and the light chain variable region (V_{L}) comprises the sequence shown in SEQ ID NO:390, or conservative amino acid substitutions thereof. In an embodiment, an anti-TF scFv domain comprises V_{H} and V_{L} regions that are each at least 99% identical to the sequences shown in SEQ ID NO:389 and SEQ ID NO:390, respectively. In an embodiment, an anti-TF scFv domain comprises V_{H} and V_{L} regions that are each at least 98% identical to the sequences shown in SEQ ID NO:389 and SEQ ID NO:390, respectively. In an embodiment, an anti-TF scFv domain comprises V_{H} and V_{L} regions that are each at least 97% identical to the sequences shown in SEQ ID NO:389 and SEQ ID NO:390, respectively. In an embodiment, an anti-TF scFv domain comprises V_{H} and V_{L} regions that are each at least 96% identical to the sequences shown in SEQ ID NO:389 and SEQ ID NO:390, respectively. In an embodiment, an anti-TF scFv domain comprises V_{H} and V_{L} regions that are each at least 95% identical to the sequences shown in SEQ ID NO:389 and SEQ ID NO:390, respectively. In an embodiment, an anti-TF scFv domain comprises V_{H} and V_{L} regions that are each at least 90% identical to the sequences shown in SEQ ID NO:389 and SEQ ID NO:390, respectively. In an embodiment, an anti-TF scFv domain comprises V_{H} and V_{L} regions that are each at least 85% identical to the sequences shown in SEQ ID NO:389 and SEQ ID NO:390, respectively. In an embodiment, an anti-TF scFv domain comprises V_{H} and V_{L} regions that are each at least 80% identical to the sequences shown in SEQ ID NO:389 and SEQ ID NO:390, respectively.

In an embodiment, an anti-TF scFv domain comprises a heavy chain variable region (V_{H}) domain and/or a light chain variable region (V_{L}) domain of the scFv antibody TF277, or variants, derivatives, fragments, or conservative amino acid substitutions thereof. In an embodiment, an anti-TF scFv domain comprises a V_{H} domain and a V_{L} domain, wherein the V_{H} domain comprises the sequence shown in SEQ ID NO:391, or conservative amino acid substitutions thereof, and the light chain variable region (V_{L}) comprises the sequence shown in SEQ ID NO:392, or conservative amino acid substitutions thereof. In an embodiment, an anti-TF scFv domain comprises V_{H} and V_{L} regions that are each at least 99% identical to the sequences shown in SEQ ID NO:391 and SEQ ID NO:392, respectively. In an embodiment, an anti-TF scFv domain comprises V_{H} and V_{L} regions that are each at least 98% identical to the sequences shown in SEQ ID NO:391 and SEQ ID NO:392, respectively. In an embodiment, an anti-TF scFv domain comprises V_{H} and V_{L} regions that are each at least 97% identical to the sequences shown in SEQ ID NO:391 and SEQ ID NO:392, respectively. In an embodiment, an anti-TF scFv domain comprises V_{H} and V_{L} regions that are each at least 96% identical to the sequences shown in SEQ ID NO:391 and SEQ ID NO:392, respectively. In an embodiment, an anti-TF scFv domain comprises V_{H} and V_{L} regions that are each at least 95% identical to the sequences shown in SEQ ID NO:391 and SEQ ID NO:392, respectively. In an embodiment, an anti-TF scFv domain comprises V_{H} and V_{L} regions that are each at least 90% identical to the sequences shown in SEQ ID NO:391 and SEQ ID NO:392, respectively. In an embodiment, an anti-TF scFv domain comprises V_{H} and V_{L} regions that are each at least 85% identical to the sequences shown in SEQ ID NO:391 and SEQ ID NO:392, respectively. In an embodiment, an anti-TF scFv domain comprises V_{H} and V_{L} regions that are each at least 80% identical to the sequences shown in SEQ ID NO:391 and SEQ ID NO:392, respectively.

In an embodiment, an anti-TF scFv domain comprises a heavy chain variable region (V_{H}) domain and/or a light chain variable region (V_{L}) domain of the scFv antibody TF394, or variants, derivatives, fragments, or conservative amino acid substitutions thereof. In an embodiment, an anti-TF scFv domain comprises a V_{H} domain and a V_{L} domain, wherein the V_{H} domain comprises the sequence shown in SEQ ID NO:393, or conservative amino acid substitutions thereof, and the light chain variable region (V_{L}) comprises the sequence shown in SEQ ID NO:394, or conservative amino acid substitutions thereof. In an embodiment, an anti-TF scFv domain comprises V_{H} and V_{L} regions that are each at least 99% identical to the sequences shown in SEQ ID NO:393 and SEQ ID NO:394, respectively. In an embodiment, an anti-TF scFv domain comprises V_{H} and V_{L} regions that are each at least 98% identical to the sequences shown in SEQ ID NO:393 and SEQ ID NO:394, respectively. In an embodiment, an anti-TF scFv domain comprises V_{H} and V_{L} regions that are each at least 97% identical to the sequences shown in SEQ ID NO:393 and SEQ ID NO:394, respectively. In an embodiment, an anti-TF scFv domain comprises V_{H} and V_{L} regions that are each at least 96% identical to the sequences shown in SEQ ID NO:393 and SEQ ID NO:394, respectively. In an embodiment, an anti-TF scFv domain comprises V_{H} and V_{L} regions that are each at least 95% identical to the sequences shown in SEQ ID NO:393 and SEQ ID NO:394, respectively. In an embodiment, an anti-TF scFv domain comprises V_{H} and V_{L} regions that are each at least 90% identical to the sequences shown in SEQ ID NO:393 and SEQ ID NO:394, respectively. In an embodiment, an anti-TF scFv domain comprises V_{H} and V_{L} regions that are each at least 85% identical to the sequences shown in SEQ ID NO:393 and SEQ ID NO:394, respectively. In an embodiment, an anti-TF scFv domain comprises V_{H} and V_{L} regions that are each at least 80% identical to the sequences shown in SEQ ID NO:393 and SEQ ID NO:394, respectively.

In an embodiment, an anti-TF scFv domain comprises a heavy chain variable region (V_{H}) domain and/or a light chain variable region (V_{L}) domain of the scFv antibody TF9, or variants, derivatives, fragments, or conservative amino acid substitutions thereof. In an embodiment, an anti-TF scFv domain comprises a V_{H} domain and a V_{L} domain, wherein the V_{H} domain comprises the sequence shown in SEQ ID NO:395, or conservative amino acid substitutions thereof, and the light chain variable region (V_{L}) comprises the sequence shown in SEQ ID NO:396, or conservative amino acid substitutions thereof. In an embodiment, an anti-TF scFv domain comprises V_{H} and V_{L} regions that are each at least 99% identical to the sequences shown in SEQ ID NO:395 and SEQ ID NO:396, respectively. In an embodiment, an anti-TF scFv domain comprises V_{H} and V_{L} regions that are each at least 98% identical to the sequences shown in SEQ ID NO:395 and SEQ ID NO:396, respectively. In an embodiment, an anti-TF scFv domain comprises V_{H} and V_{L} regions that are each at least 97% identical to the sequences shown in SEQ ID NO:395 and SEQ ID NO:396, respectively. In an embodiment, an anti-TF scFv domain comprises V_{H} and V_{L} regions that are each at least 96% identical to the sequences shown in SEQ ID NO:395 and SEQ ID NO:396, respectively. In an embodiment, an anti-TF scFv domain comprises V_{H} and V_{L} regions that are each at least 95% identical to the sequences shown in SEQ ID NO:395 and SEQ ID NO:396, respectively. In an embodiment, an anti-TF scFv domain comprises V_{H} and V_{L} regions that are each at least 90% identical to the sequences shown in SEQ ID NO:395 and SEQ ID NO:396, respectively. In an embodiment, an anti-TF scFv domain comprises V_{H} and V_{L} regions that are each at least 85% identical to the sequences shown in SEQ ID NO:395 and SEQ ID NO:396, respectively. In an embodiment, an anti-TF scFv domain comprises V_{H} and V_{L} regions that are each at least 80% identical to the sequences shown in SEQ ID NO:395 and SEQ ID NO:396, respectively.

In an embodiment, an anti-TF scFv domain comprises a V_{H} domain and a V_{L} domain, wherein the V_{H} domain comprises a sequence selected from the group consisting of SEQ ID NO:385, SEQ ID NO:387, SEQ ID NO:389, SEQ ID NO:391, SEQ ID NO:393, SEQ ID NO:395, and conservative amino acid substitutions thereof, and the light chain variable region (V_{L}) comprises a sequence selected from the group consisting of SEQ ID NO:386, SEQ ID NO:388, SEQ ID NO:390, SEQ ID NO:392, SEQ ID NO:394, SEQ ID NO:396, and conservative amino acid substitutions thereof.

In an embodiment, an anti-TF scFv domain comprises the heavy chain CDR1, CDR2 and CDR3 domains having the sequences set forth in SEQ ID NO:397, SEQ ID NO:398, and/or SEQ ID NO:399, respectively, or conservative amino acid substitutions thereof, and/or light chain CDR1, CDR2 and CDR3 domains having the sequences set forth in SEQ ID NO:400, SEQ ID NO:401, and/or SEQ ID NO:402, respectively, or conservative amino acid substitutions thereof.

In an embodiment, an anti-TF scFv domain comprises the heavy chain CDR1, CDR2 and CDR3 domains having the sequences set forth in SEQ ID NO:403, SEQ ID NO:404, and/or SEQ ID NO:405, respectively, or conservative amino acid substitutions thereof, and/or light chain CDR1, CDR2 and CDR3 domains having the sequences set forth in SEQ ID NO:406, SEQ ID NO:407, and/or SEQ ID NO:408, respectively, or conservative amino acid substitutions thereof.

In an embodiment, an anti-TF scFv domain comprises the heavy chain CDR1, CDR2 and CDR3 domains having the sequences set forth in SEQ ID NO:409, SEQ ID NO:410, and/or SEQ ID NO:411, respectively, or conservative amino acid substitutions thereof, and/or light chain CDR1, CDR2 and CDR3 domains having the sequences set forth in SEQ ID NO:412, SEQ ID NO:413, and/or SEQ ID NO:414, respectively, or conservative amino acid substitutions thereof.

In an embodiment, an anti-TF scFv domain comprises a heavy chain variable region (V_{H}) domain and/or a light chain variable region (V_{L}) domain, wherein the V_{H} domain comprises the sequence shown in SEQ ID NO:415, or conservative amino acid substitutions thereof, and the light chain variable region (V_{L}) comprises the sequence shown in SEQ ID NO:416, or conservative amino acid substitutions thereof. In an embodiment, an anti-TF scFv domain comprises V_{H} and V_{L} regions that are each at least 99% identical to the sequences shown in SEQ ID NO:415 and SEQ ID NO:416, respectively. In an embodiment, an anti-TF scFv domain comprises V_{H} and V_{L} regions that are each at least 98% identical to the sequences shown in SEQ ID NO:415 and SEQ ID NO:416, respectively. In an embodiment, an anti-TF scFv domain comprises V_{H} and V_{L} regions that are each at least 97% identical to the sequences shown in SEQ ID NO:415 and SEQ ID NO:416, respectively. In an embodiment, an anti-TF scFv domain comprises V_{H} and V_{L} regions that are each at least 96% identical to the sequences shown in SEQ ID NO:415 and SEQ ID NO:416, respectively. In an embodiment, an anti-TF scFv domain comprises V_{H} and V_{L} regions that are each at least 95% identical to the sequences shown in SEQ ID NO:415 and SEQ ID NO:416, respectively. In an embodiment, an anti-TF scFv domain comprises V_{H} and V_{L} regions that are each at least 90% identical to the sequences shown in SEQ ID NO:415 and SEQ ID NO:416, respectively. In an embodiment, an anti-TF scFv domain comprises V_{H} and V_{L} regions that are each at least 85% identical to the sequences shown in SEQ ID NO:415 and SEQ ID NO:416, respectively. In an embodiment, an anti-TF scFv domain comprises V_{H} and V_{L} regions that are each at least 80% identical to the sequences shown in SEQ ID NO:415 and SEQ ID NO:416, respectively.

In an embodiment, an anti-TF scFv domain comprises a heavy chain variable region (V_{H}) domain and/or a light chain variable region (V_{L}) domain, wherein the V_{H} domain comprises the sequence shown in SEQ ID NO:417, or conservative amino acid substitutions thereof, and the light chain variable region (V_{L}) comprises the sequence shown in SEQ ID NO:418, or conservative amino acid substitutions thereof. In an embodiment, an anti-TF scFv domain comprises V_{H} and V_{L} regions that are each at least 99% identical to the sequences shown in SEQ ID NO:417 and SEQ ID NO:418, respectively. In an embodiment, an anti-TF scFv domain comprises V_{H} and V_{L} regions that are each at least 98% identical to the sequences shown in SEQ ID NO:417 and SEQ ID NO:418, respectively. In an embodiment, an anti-TF scFv domain comprises V_{H} and V_{L} regions that are each at least 97% identical to the sequences shown in SEQ ID NO:417 and SEQ ID NO:418, respectively. In an embodiment, an anti-TF scFv domain comprises V_{H} and V_{L} regions that are each at least 96% identical to the sequences shown in SEQ ID NO:417 and SEQ ID NO:418, respectively. In an embodiment, an anti-TF scFv domain comprises V_{H} and V_{L} regions that are each at least 95% identical to the sequences shown in SEQ ID NO:417 and SEQ ID NO:418, respectively. In an embodiment, an anti-TF scFv domain comprises V_{H} and V_{L} regions that are each at least 90% identical to the sequences shown in SEQ ID NO:417 and SEQ ID NO:418, respectively. In an embodiment, an anti-TF scFv domain comprises V_{H} and V_{L} regions that are each at least 85% identical to the sequences shown in SEQ ID NO:417 and SEQ ID NO:418, respectively. In an embodiment, an anti-TF scFv domain comprises V_{H} and V_{L} regions that are each at least 80% identical to the sequences shown in SEQ ID NO:417 and SEQ ID NO:418, respectively.

In an embodiment, an anti-TF scFv domain comprises a heavy chain variable region (V_{H}) domain and/or a light chain variable region (V_{L}) domain, wherein the V_{H} domain comprises the sequence shown in SEQ ID NO:419, or conservative amino acid substitutions thereof, and the light chain variable region (V_{L}) comprises the sequence shown in SEQ ID NO:420, or conservative amino acid substitutions thereof. In an embodiment, an anti-TF scFv domain comprises V_{H} and V_{L} regions that are each at least 99% identical to the sequences shown in SEQ ID NO:419 and SEQ ID NO:420, respectively. In an embodiment, an anti-TF scFv domain comprises V_{H} and V_{L} regions that are each at least 98% identical to the sequences shown in SEQ ID NO:419 and SEQ ID NO:420, respectively. In an embodiment, an anti-TF scFv domain comprises V_{H} and V_{L} regions that are each at least 97% identical to the sequences shown in SEQ ID NO:419 and SEQ ID NO:420, respectively. In an embodiment, an anti-TF scFv domain comprises V_{H} and V_{L} regions that are each at least 96% identical to the sequences shown in SEQ ID NO:419 and SEQ ID NO:420, respectively. In an embodiment, an anti-TF scFv domain comprises V_{H} and V_{L} regions that are each at least 95% identical to the sequences shown in SEQ ID NO:419 and SEQ ID NO:420, respectively. In an embodiment, an anti-TF scFv domain comprises V_{H} and V_{L} regions that are each at least 90% identical to the sequences shown in SEQ ID NO:419 and SEQ ID NO:420, respectively. In an embodiment, an anti-TF scFv domain comprises V_{H} and V_{L} regions that are each at least 85% identical to the sequences shown in SEQ ID NO:419 and SEQ ID NO:420, respectively. In an embodiment, an anti-TF scFv domain comprises V_{H} and V_{L} regions that are each at least 80% identical to the sequences shown in SEQ ID NO:419 and SEQ ID NO:420, respectively.

In an embodiment, an anti-TF scFv domain comprises a heavy chain variable region (V_{H}) domain and/or a light chain variable region (V_{L}) domain, wherein the V_{H} domain comprises the sequence shown in SEQ ID NO:421, or conservative amino acid substitutions thereof, and the light chain variable region (V_{L}) comprises the sequence shown in SEQ ID NO:422, or conservative amino acid substitutions thereof. In an embodiment, an anti-TF scFv domain comprises V_{H} and V_{L} regions that are each at least 99% identical to the sequences shown in SEQ ID NO:421 and SEQ ID NO:422, respectively. In an embodiment, an anti-TF scFv domain comprises V_{H} and V_{L} regions that are each at least 98% identical to the sequences shown in SEQ ID NO:421 and SEQ ID NO:422, respectively. In an embodiment, an anti-TF scFv domain comprises V_{H} and V_{L} regions that are each at least 97% identical to the sequences shown in SEQ ID NO:421 and SEQ ID NO:422, respectively. In an embodiment, an anti-TF scFv domain comprises V_{H} and V_{L} regions that are each at least 96% identical to the sequences shown in SEQ ID NO:421 and SEQ ID NO:422, respectively. In an embodiment, an anti-TF scFv domain comprises V_{H} and V_{L} regions that are each at least 95% identical to the sequences shown in SEQ ID NO:421 and SEQ ID NO:422, respectively. In an embodiment, an anti-TF scFv domain comprises V_{H} and V_{L} regions that are each at least 90% identical to the sequences shown in SEQ ID NO:421 and SEQ ID NO:422, respectively. In an embodiment, an anti-TF scFv domain comprises V_{H} and V_{L} regions that are each at least 85% identical to the sequences shown in SEQ ID NO:421 and SEQ ID NO:422, respectively. In an embodiment, an anti-TF scFv domain comprises V_{H} and V_{L} regions that are each at least 80% identical to the sequences shown in SEQ ID NO:421 and SEQ ID NO:422, respectively.

In an embodiment, an anti-TF scFv domain comprises a V_{H} domain and a V_{L} domain, wherein the V_{H} domain comprises a sequence selected from the group consisting of SEQ ID NO:415, SEQ ID NO:417, SEQ ID NO:419, SEQ ID NO:421, and conservative amino acid substitutions thereof, and the light chain variable region (V_{L}) comprises a sequence selected from the group consisting of SEQ ID NO:416, SEQ ID NO:418, SEQ ID NO:420, SEQ ID NO:422, and conservative amino acid substitutions thereof.

In an embodiment, an anti-TF scFv domain comprises a heavy chain variable region (V_{H}) domain and/or a light chain variable region (V_{L}) domain, wherein the V_{H} domain comprises the sequence shown in SEQ ID NO:423, or conservative amino acid substitutions thereof, and the light chain variable region (V_{L}) comprises the sequence shown in SEQ ID NO:424, or conservative amino acid substitutions thereof. In an embodiment, an anti-TF scFv domain comprises V_{H} and V_{L} regions that are each at least 99% identical to the sequences shown in SEQ ID NO:423 and SEQ ID NO:424, respectively. In an embodiment, an anti-TF scFv domain comprises V_{H} and V_{L} regions that are each at least 98% identical to the sequences shown in SEQ ID NO:423 and SEQ ID NO:424, respectively. In an embodiment, an anti-TF scFv domain comprises V_{H} and V_{L} regions that are each at least 97% identical to the sequences shown in SEQ ID NO:423 and SEQ ID NO:424, respectively. In an embodiment, an anti-TF scFv domain comprises V_{H} and V_{L} regions that are each at least 96% identical to the sequences shown in SEQ ID NO:423 and SEQ ID NO:424, respectively. In an embodiment, an anti-TF scFv domain comprises V_{H} and V_{L} regions that are each at least 95% identical to the sequences shown in SEQ ID NO:423 and SEQ ID NO:424, respectively. In an embodiment, an anti-TF scFv domain comprises V_{H} and V_{L} regions that are each at least 90% identical to the sequences shown in SEQ ID NO:423 and SEQ ID NO:424, respectively. In an embodiment, an anti-TF scFv domain comprises V_{H} and V_{L} regions that are each at least 85% identical to the sequences shown in SEQ ID NO:423 and SEQ ID NO:424, respectively. In an embodiment, an anti-TF scFv domain comprises V_{H} and V_{L} regions that are each at least 80% identical to the sequences shown in SEQ ID NO:423 and SEQ ID NO:424, respectively.

The nucleotide sequences encoding exemplary TF binding V_{H} and V_{L} domains for scFv domains for TF260, TF196, TF278, TF277, TF392, and TF9 are provided in Table 49 and are further described in U.S. Pat. No. 7,993,644, the disclosures of which are incorporated by reference herein. In an embodiment, a nucleotide sequence in Table 49 is codon-optimized to improve protein expression.

TABLE 49
Nucleotide sequences of exemplary tissue-factor binding scFv domains.

| Identifier | Sequence (One-Letter Nucleotide Symbols) | |
|---|---|---|
| SEQ ID NO: 425 | CAGGTGCAGC TGAAGCAGTC TGGAGCTGAG CTGATGAAGC CTGGGGCCTC AGTGAAGATA | 60 |
| Anti-TF variable | TCCTGCAAGG CTACTGGCTA CACATTCAGT AGCTACTGGA TAGAGTGGGT AAAGCAGAGG | 120 |
| heavy chain | CCTGGACATG GCCTTGAGTG GATTGGAGAG ATTTTACCTG GAAGTGGTAG TACTAACTAC | 180 |
| TF260 | AATGAGAAGT TCAAGGGCAA GGCCACATTC ACTGCAGATA CATCCTCCAA CACAGCCTAC | 240 |
| | ATGCAACTCA GCAGCCTGAC ATCTGAGGAC TCTGCCGTCT ATTACTGTGC AAGAGAGGAT | 300 |
| | AGGTACGACG GTGACTACTG GGGCCAAGGC ACCACTCTCA CAGTCTCGAG | 350 |
| SEQ ID NO: 426 | CAGGCTGTTG TGACTCAGGA ATCTGCACTC ACCACATCAC CTGGTGAAAC AGTCACACTC | 60 |
| Anti-TF variable | ACTTGTCGCT CAAGTACTGG GGCTGTTACA ACTAGTAACT ATGCCAACTG GGTCCAAGAA | 120 |
| light chain | AAACCAGATC ATTTATTCAC TGGTCTAATA GGTGGTACCA ACAACCGAGC TCCAGGTGTT | 180 |
| TF260 | CCTGCCAGAT TCTCAGGCTC CCTGATTGGA GACAAGGCTG CCCTCACCAT CACAGGGGCA | 240 |
| | CAGACTGAGG ATGAGGCAAT ATATTTCTGT GCTCTATGGT ACAGCAACCA CTGGGTGTTC | 300 |
| | GGTGGAGGAA CCAAACTGAC TGTCCTAGGT CAGCCCC | 337 |
| SEQ ID NO: 427 | CAGGTGCAGC TGAAGCAGTC TGGACCTGAG CTGGAGAAGC CTGGCGCTTC AGTGAAGATA | 60 |
| Anti-TF variable | TCCTGCAAGG CTTCTGGTTA CTCATTCACT GGCTACAACA TGAACTGGGT GAAGCAGAGC | 120 |
| heavy chain | AATGGAAAGA GCCTTGAGTG GATTGGAAAT ATTGATCCTT ACTATGGTGG TACTAGCTAC | 180 |
| TF196 | AACCAGAAGT TCAAGGGCAA GGCCACATTG ACTGTAGACA AATCCTCCAA CACAGCCTAC | 240 |
| | ATGCACCTCA AGAGCCTGAC ATCTGAGGAC TCTGCAGTCT ATTACTGTGC AAGAGATAGT | 300 |
| | AGCTCCTGGT TTGCTTACTG GGGCCAAGGG ACTCTGGTCA CTGTCTCTGC A | 351 |
| SEQ ID NO: 428 | GACATCCAGC TGACTCAGTC TCCAGCCTCC CTATCTGCAT CTGEGGGAGA AACTGTCACC | 60 |
| Anti-TF variable | ATCACATGTC GAGCAAGTGG GAATATTCAC AATTATTTAG CATGGTATCA GCAGAAACAG | 120 |
| light chain | GGAAAATCTC CTCAGCTCCT GGTCTATAAT GCAAAAACCT TAGCAGATGG TGTGCCATCA | 180 |
| TF196 | AGGTTCAGTG GCAGTGGATC AGGAACACAA TATTCTCTCA AGATCAACAG CCTGCAGCCT | 240 |
| | GAAGATTTTG GGAGTTATTA CTGTCAACAT TTTTGGATTA CTCCGTGGAC GTTCGGTGGA | 300 |
| | GGCACCAAGC TGGAGATCTA ACGGA | 325 |
| SEQ ID NO: 429 | GAGGTCCAGC TGCAGCAATC TGGAGCTGAG CTGATGAAGC CTGGGGCCTC AGTGAAGATA | 60 |
| Anti-TF variable | TCCTGCAAGG CTACTGGCTA CACATTCAGT AGCTACTGGA TAGAGTGGGT AAAGCAGAGG | 120 |
| heavy chain | CCTGGACATG GCCTTGAGTG GATTGGAGAG ATTTTACCTG GAAGTGCTAG TACTAAGTAC | 180 |
| TF278 | AATGAGAAGT TCAAGGGCAA GGCCACATTC ACTGCAGATA CATCCTCCAA CACAGCCTAC | 240 |
| | ATGCAACTCA GCAGCCTGAC ATCTGAGGAC TCTGCCGTCT ATTACTGTGC AAGAGATTAT | 300 |
| | TACTACGGTA GTAGCTACGG GTTTGCTTAC TGGGGCCAAG GGACTCTGGT CACTGTCTCG | 360 |
| | AGT | 363 |
| SEQ ID NO: 430 | CAGGCTGTTG TGACTCAGGA ATCTGCACTC ACCACATCAC CTGGTGAAAC AGTCACACTC | 60 |
| Anti-TF variable | ACTTGTCGCT CAAGTACTGG GGCTGTTACA ACTAGTAACT ATGCCAACTG GGTCCAAGAA | 120 |
| light chain | AAACCAGATC ATTTATTCAC TGGCCTAATA GGTGGTACCA ACAACCGAGG TCCAGGTGTT | 180 |
| TF278 | CCTGCCAGAT TCTCAGGCTC CCTGATTGGA GACAAGGCTG CCCCACCAT CACAGGGGCA | 240 |
| | CAGACTGAGG ATGAGGCAGT ATATTTCTGT GCTCTATGGT ACAGCAACCA TTGGGTGTTC | 300 |
| | GGTGGAGGAA CCAAACTGAC TGTCCTAGGT | 330 |
| SEQ ID NO: 431 | CAGGTCCAAC TGCAGCAGCC TGGGGCTGAG CTTGTGAAGC CTGGGGCTTC AGTGAAGCTG | 60 |
| Anti-TF variable | TCCTGCAAGA CTTCTGGCTA CACCTTCACC AGCTACTGGA TGCACTGGGT GAAGCAGAGG | 120 |
| heavy chain | CCTGGACAAG GCCTTGAGTG GATCGGAGAG ATTGATCCTT CTGATAGTTA TACTAACTAC | 180 |
| TF277 | AATCAAAAGT TCAAGGGCAA GGCCACATTG ACTGTAGACA AATCCTCCAG CACAGCCTAC | 240 |
| | ATGCAGCTCA GCAGCCTGAC ATCTGAGGAC TCTGCGGTCT ATTACTGTAC CTACTATGTT | 300 |
| | AACTACTATG CTATGGACTA CTGGGGTCAA GGAACCTCAG TCACCGTCTC CTCA | 354 |
| SEQ ID NO: 432 | CAAATTGTTC TCACCCAGTC TCCAGCAATC ATGTCTGCAT CTCTAGGGGA GGAGATCACC | 60 |
| Anti-TF variable | CTAACCTGCA GTGCCAGCTC GAGTGTAAGT TACATGCACT GGTACCAGCA GAAGTCAGGC | 120 |
| light chain | ACTTCTCCCA AACTCTTGAT TTATAGCACA TCCAACCTGG CTTCTGGAGT CCCTTCTCGC | 180 |
| TF277 | TTCAGTGGCA GTGGGTCTGG GACCTTTTAT TCTCTCACAA TCAGCAGTGT GGAGGCTGAA | 240 |
| | GATGCTGCCG ATTATTACTG CCATCAGTGG AGTAGTTATC CATACACGTT CGGAGGGGGG | 300 |
| | ACCAAGCTGG AAATAAAA | 318 |
| SEQ ID NO: 433 | CAGGTGCAGC TGAAGGAGTC TGGAGCTGAG CTGATGAAGC CTGGGGCCTC AGTGAAGATA | 60 |
| Anti-TF variable | TCCTGCAAGG CTACTGGCTA CACATTCAGT AGCTACTGGA TAGAGTGGGT AAAGCAGAGG | 120 |
| heavy chain | CCTGGACATG GCCTTGAGTG GATTGGAGAG ATTTTACCTG GAAGTGGTAG TACTAACTAC | 180 |
| TF392 | AATGAGAAGT TCAAGGGCAA GGCCACATTC ACTGCAGATA CATCCTCCAA CACAGCCTAC | 240 |
| | ATGCAACTCA GCAGCCTGAC ATCTGAGGAC TCTGCCGTCT ATTACTGTGC AAGAGACAGG | 300 |
| | AACGGCTACG TGAACTACTT TGACTCCTGG GGCCAAGGCA CCACTCTCAC AGTCTCCTCA | 360 |
| SEQ ID NO: 434 | CAGGCTGTTG TGACTCAGGA ATCTGCACTC ACCACATCAC CTGGTGAAAC AGTCACACTC | 60 |
| Anti-TF variable | ACTTGTCGCT CAAGTACTGG GGCTGTTACA ACTAGTAACT ATGCCAACTG GGTCCAAGAA | 120 |
| light chain | AAACCAGATC ATTTATTCAC TGGTCTAATA GGTGGTACCA ACAACCGAGC TCCAGGTGTT | 180 |
| TF392 | CCTGCCAGAT TCTCAGGCTC CCTGATTGGA GACAAGGCTG CCCSCACCAT CACAGGGGCA | 240 |
| | CAGACTGAGG ATGAGGCAAT ATATTTCTGT GCTCTATGGT ACAGCAACCA CTGGGTGTTC | 300 |
| | GGTGGAGGAA CCAAACTGAC TGTCCTAGGT CAGCCCC | 337 |
| SEQ ID NO: 435 | GATGTGAAGC TTCAGGAGTC AGGACCTGAC CTGGTGAAAC CTTCTCAGTC ACTTTCACTC | 60 |
| Anti-TF variable | ACCTGCACTG TCACTGGCTA CTCCATCACC AGTGGTTATA GCTGGCACTG GATCCGGCAG | 120 |
| heavy chain TF9 | TTTCCAGGAA ACAAACTGGA ATGGATGGGC TACATACACT ACAGTGGTAG CACTAAGTAC | 180 |
| | AACCCATCTC TCAAAAGTCG AATCTCTATC ACTCGAGACA CATCCAAGAA CCAGTTCTTC | 240 |
| | CTGCAGTTGA ATTCTGTGAC TACTGAGGAC ACAGCCACAT ATTACTGTGC AAGACTCTGG | 300 |
| | AGTTGGTACT TCGATGTCTG GGGCGCAGGG ACCACGGTCA CCGTCTCCTC A | 351 |
| SEQ ID NO: 436 | AACATTATGA TGACACAGTC GCCATCATCT CTGGCTGTGT CTGCAGGAGA AAAGGTCACT | 60 |
| Anti-TF variable | ATGAGCTGTA AGTCCAGTCA AAGTGTTTTA TACAGTTCAA ATCAGAAGAA CTACTTGGCC | 120 |
| light chain TF9 | TGGTACCAGC AGAAACCAGG GCAGTCTCCT AAACTGCTGA TCTACTGGGC ATCCACTAGG | 180 |
| | GAATCTGGTG TCCCTGATCG CTTCACAGGC AGTGGATCTG GGACAGATTT TACTCTTACC | 240 |
| | ATCAGCAGTG TACAAGCTGA AGACCTGGCA GTTTATTACT GTCATCAATA CCTCTCCTCG | 300 |
| | TACACGTTCG GAGGGGGGAC CAAGCTGGAA ATAAAA | 336 |

In an embodiment, an anti-TF scFv domain comprises a heavy chain variable region (V_{H}) domain and/or a light chain variable region (V_{L}) domain of the antibody TF260 encoded by a nucleotide sequence. In an embodiment, an anti-TF scFv domain comprises a V_{H} domain and/or a V_{L} domain, wherein the V_{H} domain is encoded by the sequence shown in SEQ ID NO:425 and the light chain variable region (V_{L}) is encoded by the sequence shown in SEQ ID NO:426. In an embodiment, an anti-TF scFv domain comprises V_{H} and/or V_{L} regions that are encoded by nucleotides that are each at least 99% identical to the sequences shown in SEQ ID NO:425 and SEQ ID NO:426, respectively. In an embodiment, an anti-TF scFv domain comprises V_{H} and/or V_{L} regions that are encoded by nucleotides that are each at least 98% identical to the sequences shown in SEQ ID NO:425 and SEQ ID NO:426, respectively. In an embodiment, an anti-TF scFv domain comprises V_{H} and/or V_{L} regions that are encoded by nucleotides that are each at least 97% identical to the sequences shown in SEQ ID NO:425 and SEQ ID NO:426, respectively. In an embodiment, an anti-TF scFv domain comprises V_{H} and/or V_{L} regions that are encoded by nucleotides that are each at least 96% identical to the sequences shown in SEQ ID NO:425 and SEQ ID NO:426, respectively. In an embodiment, an anti-TF scFv domain comprises V_{H} and/or V_{L} regions that are encoded by nucleotides that are each at least 95% identical to the sequences shown in SEQ ID NO:425 and SEQ ID NO:426, respectively. In an embodiment, an anti-TF scFv domain comprises V_{H} and/or V_{L} regions that are encoded by nucleotides that are each at least 90% identical to the sequences shown in SEQ ID NO:425 and SEQ ID NO:426, respectively. In an embodiment, an anti-TF scFv domain comprises V_{H} and/or V_{L} regions that are encoded by nucleotides that are each at least 85% identical to the sequences shown in SEQ ID NO:425 and SEQ ID NO:426, respectively. In an embodiment, an anti-TF scFv domain comprises V_{H} and/or V_{L} regions that are encoded by nucleotides that are each at least 80% identical to the sequences shown in SEQ ID NO:425 and SEQ ID NO:426, respectively. In an embodiment, including the foregoing embodiments, SEQ ID NO:425 and/or SEQ ID NO:426 is codon-optimized to improve protein expression.

In an embodiment, an anti-TF scFv domain comprises a heavy chain variable region (V_{H}) domain and/or a light chain variable region (V_{L}) domain of the antibody TF196 encoded by a nucleotide sequence. In an embodiment, an anti-TF scFv domain comprises a V_{H} domain and/or a V_{L} domain, wherein the V_{H} domain is encoded by the sequence shown in SEQ ID NO:427 and the light chain variable region (V_{L}) is encoded by the sequence shown in SEQ ID NO:428. In an embodiment, an anti-TF scFv domain comprises V_{H} and/or V_{L} regions that are encoded by nucleotides that are each at least 99% identical to the sequences shown in SEQ ID NO:427 and SEQ ID NO:428, respectively. In an embodiment, an anti-TF scFv domain comprises V_{H} and/or V_{L} regions that are encoded by nucleotides that are each at least 98% identical to the sequences shown in SEQ ID NO:427 and SEQ ID NO:428, respectively. In an embodiment, an anti-TF scFv domain comprises V_{H} and/or V_{L} regions that are encoded by nucleotides that are each at least 97% identical to the sequences shown in SEQ ID NO:427 and SEQ ID NO:428, respectively. In an embodiment, an anti-TF scFv domain comprises V_{H} and/or V_{L} regions that are encoded by nucleotides that are each at least 96% identical to the sequences shown in SEQ ID NO:427 and SEQ ID NO:428, respectively. In an embodiment, an anti-TF scFv domain comprises V_{H} and/or V_{L} regions that are encoded by nucleotides that are each at least 95% identical to the sequences shown in SEQ ID NO:427 and SEQ ID NO:428, respectively. In an embodiment, an anti-TF scFv domain comprises V_{H} and/or V_{L} regions that are encoded by nucleotides that are each at least 90% identical to the sequences shown in SEQ ID NO:427 and SEQ ID NO:428, respectively. In an embodiment, an anti-TF scFv domain comprises V_{H} and/or V_{L} regions that are encoded by nucleotides that are each at least 85% identical to the sequences shown in SEQ ID NO:427 and SEQ ID NO:428, respectively. In an embodiment, an anti-TF scFv domain comprises V_{H} and/or V_{L} regions that are encoded by nucleotides that are each at least 80% identical to the sequences shown in SEQ ID NO:427 and SEQ ID NO:428, respectively. In an embodiment, including the foregoing embodiments, SEQ ID NO:427 and/or SEQ ID NO:428 is codon-optimized to improve protein expression.

In an embodiment, an anti-TF scFv domain comprises a heavy chain variable region (V_{H}) domain and/or a light chain variable region (V_{L}) domain of the antibody TF278 encoded by a nucleotide sequence. In an embodiment, an anti-TF scFv domain comprises a V_{H} domain and/or a V_{L} domain, wherein the V_{H} domain is encoded by the sequence shown in SEQ ID NO:429 and the light chain variable region (V_{L}) is encoded by the sequence shown in SEQ ID NO:430. In an embodiment, an anti-TF scFv domain comprises V_{H} and/or V_{L} regions that are encoded by nucleotides that are each at least 99% identical to the sequences shown in SEQ ID NO:429 and SEQ ID NO:430, respectively. In an embodiment, an anti-TF scFv domain comprises V_{H} and/or V_{L} regions that are encoded by nucleotides that are each at least 98% identical to the sequences shown in SEQ ID NO:429 and SEQ ID NO:430, respectively. In an embodiment, an anti-TF scFv domain comprises V_{H} and/or V_{L} regions that are encoded by nucleotides that are each at least 97% identical to the sequences shown in SEQ ID NO:429 and SEQ ID NO:430, respectively. In an embodiment, an anti-TF scFv domain comprises V_{H} and/or V_{L} regions that are encoded by nucleotides that are each at least 96% identical to the sequences shown in SEQ ID NO:429 and SEQ ID NO:430, respectively. In an embodiment, an anti-TF scFv domain comprises V_{H} and/or V_{L} regions that are encoded by nucleotides that are each at least 95% identical to the sequences shown in SEQ ID NO:429 and SEQ ID NO:430, respectively. In an embodiment, an anti-TF scFv domain comprises V_{H} and/or V_{L} regions that are encoded by nucleotides that are each at least 90% identical to the sequences shown in SEQ ID NO:429 and SEQ ID NO:430, respectively. In an embodiment, an anti-TF scFv domain comprises V_{H} and/or V_{L} regions that are encoded by nucleotides that are each at least 85% identical to the sequences shown in SEQ ID NO:429 and SEQ ID NO:430, respectively. In an embodiment, an anti-TF scFv domain comprises V_{H} and/or V_{L} regions that are encoded by nucleotides that are each at least 80% identical to the sequences shown in SEQ ID NO:429 and SEQ ID NO:430, respectively. In an embodiment, including the foregoing embodiments, SEQ ID NO:429 and/or SEQ ID NO:430 is codon-optimized to improve protein expression.

In an embodiment, an anti-TF scFv domain comprises a heavy chain variable region (V_{H}) domain and/or a light chain variable region (V_{L}) domain of the antibody TF277 encoded by a nucleotide sequence. In an embodiment, an anti-TF scFv domain comprises a V_{H} domain and/or a V_{L} domain, wherein the V_{H} domain is encoded by the sequence shown in SEQ ID NO:431 and the light chain variable region (V_{L}) is encoded by the sequence shown in SEQ ID NO:432. In an embodiment, an anti-TF scFv domain comprises V_{H} and/or V_{L} regions that are encoded by nucleotides that are each at least 99% identical to the sequences shown in SEQ ID NO:431 and SEQ ID NO:432, respectively. In an embodiment, an anti-TF scFv domain comprises V_{H} and/or V_{L} regions that are encoded by nucleotides that are each at least 98% identical to the sequences shown in SEQ ID NO:431 and SEQ ID NO:432, respectively. In an embodiment, an anti-TF scFv domain comprises V_{H} and/or V_{L} regions that are encoded by nucleotides that are each at least 97% identical to the sequences shown in SEQ ID NO:431 and SEQ ID NO:432, respectively. In an embodiment, an anti-TF scFv domain comprises V_{H} and/or V_{L} regions that are encoded by nucleotides that are each at least 96% identical to the sequences shown in SEQ ID NO:431 and SEQ ID NO:432, respectively. In an embodiment, an anti-TF scFv domain comprises V_{H} and/or V_{L} regions that are encoded by nucleotides that are each at least 95% identical to the sequences shown in SEQ ID NO:431 and SEQ ID NO:432, respectively. In an embodiment, an anti-TF scFv domain comprises V_{H} and/or V_{L} regions that are encoded by nucleotides that are each at least 90% identical to the sequences shown in SEQ ID NO:431 and SEQ ID NO:432, respectively. In an embodiment, an anti-TF scFv domain comprises V_{H} and/or V_{L} regions that are encoded by nucleotides that are each at least 85% identical to the sequences shown in SEQ ID NO:431 and SEQ ID NO:432, respectively. In an embodiment, an anti-TF scFv domain comprises V_{H} and/or V_{L} regions that are encoded by nucleotides that are each at least 80% identical to the sequences shown in SEQ ID NO:431 and SEQ ID NO:432, respectively. In an embodiment, including the foregoing embodiments, SEQ ID NO:431 and/or SEQ ID NO:432 is codon-optimized to improve protein expression.

In an embodiment, an anti-TF scFv domain comprises a heavy chain variable region (V_{H}) domain and/or a light chain variable region (V_{L}) domain of the antibody TF392 encoded by a nucleotide sequence. In an embodiment, an anti-TF scFv domain comprises a V_{H} domain and/or a V_{L} domain, wherein the V_{H} domain is encoded by the sequence shown in SEQ ID NO:433 and the light chain variable region (V_{L}) is encoded by the sequence shown in SEQ ID NO:434. In an embodiment, an anti-TF scFv domain comprises V_{H} and/or V_{L} regions that are encoded by nucleotides that are each at least 99% identical to the sequences shown in SEQ ID NO:433 and SEQ ID NO:434, respectively. In an embodiment, an anti-TF scFv domain comprises V_{H} and/or V_{L} regions that are encoded by nucleotides that are each at least 98% identical to the sequences shown in SEQ ID NO:433 and SEQ ID NO:434, respectively. In an embodiment, an anti-TF scFv domain comprises V_{H} and/or V_{L} regions that are encoded by nucleotides that are each at least 97% identical to the sequences shown in SEQ ID NO:433 and SEQ ID NO:434, respectively. In an embodiment, an anti-TF scFv domain comprises V_{H} and/or V_{L} regions that are encoded by nucleotides that are each at least 96% identical to the sequences shown in SEQ ID NO:433 and SEQ ID NO:434, respectively. In an embodiment, an anti-TF scFv domain comprises V_{H} and/or V_{L} regions that are encoded by nucleotides that are each at least 95% identical to the sequences shown in SEQ ID NO:433 and SEQ ID NO:434, respectively. In an embodiment, an anti-TF scFv domain comprises V_{H} and/or V_{L} regions that are encoded by nucleotides that are each at least 90% identical to the sequences shown in SEQ ID NO:433 and SEQ ID NO:434, respectively. In an embodiment, an anti-TF scFv domain comprises V_{H} and/or V_{L} regions that are encoded by nucleotides that are each at least 85% identical to the sequences shown in SEQ ID NO:433 and SEQ ID NO:434, respectively. In an embodiment, an anti-TF scFv domain comprises V_{H} and/or V_{L} regions that are encoded by nucleotides that are each at least 80% identical to the sequences shown in SEQ ID NO:433 and SEQ ID NO:434, respectively. In an embodiment, including the foregoing embodiments, SEQ ID NO:433 and/or SEQ ID NO:434 is codon-optimized to improve protein expression.

In an embodiment, an anti-TF scFv domain comprises a heavy chain variable region (V_{H}) domain and/or a light chain variable region (V_{L}) domain of the antibody TF9 encoded by a nucleotide sequence. In an embodiment, an anti-TF scFv domain comprises a V_{H} domain and/or a V_{L} domain, wherein the V_{H} domain is encoded by the sequence shown in SEQ ID NO:435 and the light chain variable region (V_{L}) is encoded by the sequence shown in SEQ ID NO:436. In an embodiment, an anti-TF scFv domain comprises V_{H} and/or V_{L} regions that are encoded by nucleotides that are each at least 99% identical to the sequences shown in SEQ ID NO:435 and SEQ ID NO:436, respectively. In an embodiment, an anti-TF scFv domain comprises V_{H} and/or V_{L} regions that are encoded by nucleotides that are each at least 98% identical to the sequences shown in SEQ ID NO:435 and SEQ ID NO:436, respectively. In an embodiment, an anti-TF scFv domain comprises V_{H} and/or V_{L} regions that are encoded by nucleotides that are each at least 97% identical to the sequences shown in SEQ ID NO:435 and SEQ ID NO:436, respectively. In an embodiment, an anti-TF scFv domain comprises V_{H} and/or V_{L} regions that are encoded by nucleotides that are each at least 96% identical to the sequences shown in SEQ ID NO:435 and SEQ ID NO:436, respectively. In an embodiment, an anti-TF scFv domain comprises V_{H} and/or V_{L} regions that are encoded by nucleotides that are each at least 95% identical to the sequences shown in SEQ ID NO:435 and SEQ ID NO:436, respectively. In an embodiment, an anti-TF scFv domain comprises V_{H} and/or V_{L} regions that are encoded by nucleotides that are each at least 90% identical to the sequences shown in SEQ ID NO:435 and SEQ ID NO:436, respectively. In an embodiment, an anti-TF scFv domain comprises VII and/or V_{L} regions that are encoded by nucleotides that are each at least 85% identical to the sequences shown in SEQ ID NO:435 and SEQ ID NO:436, respectively. In an embodiment, an anti-TF scFv domain comprises V_{H} and/or V_{L} regions that are encoded by nucleotides that are each at least 80% identical to the sequences shown in SEQ ID NO:435 and SEQ ID NO:436, respectively. In an embodiment, including the foregoing embodiments, SEQ ID NO:435 and/or SEQ ID NO:436 is codon-optimized to improve protein expression.

### 8. Extracellular LFA-1 Binding Domains

In an embodiment, a CCR of the present invention comprises an extracellular domain, wherein the extracellular domain comprises a domain capable of binding to the T-cell integrin known as lymphocyte function-associated antigen 1 (LFA-1), also referred to herein as an anti-LFA-1 domain. The alpha subunit of LFA-1 is known as CD11a, and its ligands include ICAM-1, ICAM-2, ICAM-3, ICAM-4, ICAM-5, and JAM-A. LFA-1 is described in more detail in Walling and Kim, Front. Immunol., 2018, 9, 1-10, the disclosures of which are incorporated by reference herein. In an embodiment, the extracellular domain binds to human LFA-1. In an embodiment, the extracellular domain binds to murine LFA-1. In an embodiment, the extracellular LFA-1 binding domain is a scFv domain that binds to human LFA-1 or murine LFA-1. In an embodiment, a CCR of the present invention comprises a construct as shown in FIG. 34, wherein the V_{H} and V_{L} domains are anti-LFA-1 V_{H} and V_{L} domains, and the linker is as described herein. In an embodiment, the extracellular domain binds to human CD11a (also referred to herein as anti-CD11a). In an embodiment, the extracellular domain binds to murine CD11a. In an embodiment, the extracellular CD1 Ta binding domain is a scFv domain that binds to human CD1Ta or murine CD11a. In an embodiment, a CCR of the present invention comprises a construct as shown in FIG. 34, wherein the V_{H} and V_{L}, domains are anti-CD11a V_{H} and V_{L}, domains, and the linker is as described herein.

In some embodiments, the LFA-1 binding domain or CD11a binding domain of a CCR of the present invention includes the scFv domain of antibodies described in U.S. Patent Application Publication No. US 2015/0079075 A1. The amino acid sequences of exemplary LFA-1 or CD11a binding domains for use in the CCRs of the present invention are provided in Table 50.

TABLE 50
Amino acid sequences of exemplary LFA-1 or CD11a binding scFv domains.

| Identifier | Sequence (One-Letter Amino Acid Symbols) | |
|---|---|---|
| SEQ ID NO: 437 | EVQLVESGGG LVQPGGSLRL SCAASGFTFS SFGMEWVRQA PGKGLEWVSY ISSGSSTLHY | 60 |
| Anti-LFA-1 | ADTVKGRFTI SRDNSKNSLY LQMNSLRAED TAVYYCARGS RNLSHRLLSY WGQGTLVTVS | 120 |
| variable heavy | S | 121 |
| chain | | |
| SEQ ID NO: 438 | DIVMTQSPSL LSASVGDRVT ITCKASQDVS TAVAWYQQKP GKAPKLLIYW ASTRHTGVPS | 60 |
| Anti-LFA-1 | RFSGSGSGTD FTLTISSLQP EDFALYYCQQ HYTTPWTFGG GTKVEIKR | 108 |
| variable light | | |
| chain | | |
| SEQ ID NO: 439 | EVKLVESGGG LVQPGGSLRL SCAASGFTFS SFGMEWVRQA PEKGLEWVAY ISSGSSTLHY | 60 |
| Anti-LFA-1 | ADTVKGRFTI SRDNSKNSLY LQMNLLRAED TAVYYCARGS RNLSHRLLSY WGQGTLVTVS | 120 |
| variable heavy | S | 121 |
| chain | | |
| SEQ ID NO: 440 | DILMTQSPSL LSASVGDRVT ITCKASQDVS TAVAWYQQKP GKAPKLLIYW ASTRHTGVPS | 60 |
| Anti-LFA-1 | RFTGSGSGTD FTLTISRLQA EDFALYYCQQ HYTTPWTFGG GTKVEIKR | 108 |
| variable light | | |
| chain | | |
| SEQ ID NO: 441 | SFGMH | 5 |
| Anti-LFA-1 | | |
| variable heavy | | |
| chain CDR1 | | |
| SEQ ID NO: 442 | YISSGSSTLH YADTVKG | 17 |
| Anti-LFA-1 | | |
| variable heavy | | |
| chain CDR2 | | |
| SEQ ID NO: 443 | GSRNLSERLL S | 11 |
| Anti- LFA-1 | | |
| variable heavy | | |
| chain CDR3 | | |
| SEQ ID NO: 444 | KASQDVSTAV A | 11 |
| Anti-LFA-1 | | |
| variable heavy | | |
| chain CDR1 | | |
| SEQ ID NO: 445 | WASTRHT | 7 |
| Anti-LFA-1 | | |
| variable heavy | | |
| chain CDR2 | | |
| SEQ ID NO: 446 | QQHYTTPWT | 9 |
| Anti-LFA-1 | | |
| variable heavy | | |
| chain CDR3 | | |

In an embodiment, an anti-LFA-1 or anti-CD11a scFv domain comprises a heavy chain variable region (V_{H}) domain and/or a light chain variable region (V_{L}) domain, wherein the V_{H} domain comprises the sequence shown in SEQ ID NO:437, or conservative amino acid substitutions thereof, and the light chain variable region (V_{L}) comprises the sequence shown in SEQ ID NO:438, or conservative amino acid substitutions thereof. In an embodiment, an anti-LFA-1 or anti-CD1 Ta scFv domain comprises V_{H} and V_{L} regions that are each at least 99% identical to the sequences shown in SEQ ID NO:437 and SEQ ID NO:438, respectively. In an embodiment, an anti-LFA-1 or anti-CD1Ta scFv domain comprises V_{H} and V_{L} regions that are each at least 98% identical to the sequences shown in SEQ ID NO:437 and SEQ ID NO:438, respectively. In an embodiment, an anti-LFA-1 or anti-CD11a scFv domain comprises V_{H} and V_{L} regions that are each at least 97% identical to the sequences shown in SEQ ID NO:437 and SEQ ID NO:438, respectively. In an embodiment, an anti-LFA-1 or anti-CD1 Ta domain comprises V_{H} and V_{L} regions that are each at least 96% identical to the sequences shown in SEQ ID NO:437 and SEQ ID NO:438, respectively. In an embodiment, an anti-LFA-1 or anti-CD11a scFv domain comprises V_{H} and V_{L} regions that are each at least 95% identical to the sequences shown in SEQ ID NO:437 and SEQ ID NO:438, respectively. In an embodiment, an anti-LFA-1 or anti-CD11a scFv domain comprises V_{H} and V_{L} regions that are each at least 90% identical to the sequences shown in SEQ ID NO:437 and SEQ ID NO:438, respectively. In an embodiment, an anti-LFA-1 or anti-CD1Ta scFv domain comprises V_{H} and V_{L} regions that are each at least 85% identical to the sequences shown in SEQ ID NO:437 and SEQ ID NO:438, respectively. In an embodiment, an anti-LFA-1 or anti-CD1Ta scFv domain comprises V_{H} and V_{L} regions that are each at least 80% identical to the sequences shown in SEQ ID NO:437 and SEQ ID NO:438, respectively.

In an embodiment, an anti-LFA-1 or anti-CD11a scFv domain comprises a heavy chain variable region (V_{H}) domain and/or a light chain variable region (V_{L}) domain, wherein the V_{H} domain comprises the sequence shown in SEQ ID NO:439, or conservative amino acid substitutions thereof, and the light chain variable region (V_{L}) comprises the sequence shown in SEQ ID NO:440, or conservative amino acid substitutions thereof. In an embodiment, an anti-LFA-1 or anti-CD1 Ta scFv domain comprises V_{H} and V_{L} regions that are each at least 99% identical to the sequences shown in SEQ ID NO:439 and SEQ ID NO:440, respectively. In an embodiment, an anti-LFA-1 or anti-CD11a scFv domain comprises V_{H} and V_{L} regions that are each at least 98% identical to the sequences shown in SEQ ID NO:439 and SEQ ID NO:440, respectively. In an embodiment, an anti-LFA-1 or anti-CD11a scFv domain comprises V_{H} and V_{L} regions that are each at least 97% identical to the sequences shown in SEQ ID NO:439 and SEQ ID NO:440, respectively. In an embodiment, an anti-LFA-1 or anti-CD1 Ta scFv domain comprises V_{H} and V_{L} regions that are each at least 96% identical to the sequences shown in SEQ ID NO:439 and SEQ ID NO:440, respectively. In an embodiment, an anti-LFA-1 or anti-CD11a scFv domain comprises V_{H} and V_{L} regions that are each at least 95% identical to the sequences shown in SEQ ID NO:439 and SEQ ID NO:440, respectively. In an embodiment, an anti-LFA-1 or anti-CD11a scFv domain comprises V_{H} and V_{L} regions that are each at least 90% identical to the sequences shown in SEQ ID NO:439 and SEQ ID NO:440, respectively. In an embodiment, an anti-LFA-1 or anti-CD11a scFv domain comprises V_{H} and V_{L} regions that are each at least 85% identical to the sequences shown in SEQ ID NO:439 and SEQ ID NO:440, respectively. In an embodiment, an anti-LFA-1 or anti-CD11a scFv domain comprises V_{H} and V_{L} regions that are each at least 80% identical to the sequences shown in SEQ ID NO:439 and SEQ ID NO:440, respectively.

In an embodiment, an anti-LFA-1 or anti-CD11a scFv domain comprises a V_{H} domain and a V_{L} domain, wherein the V_{H} domain comprises a sequence selected from the group consisting of SEQ ID NO:437, SEQ ID NO:439, and conservative amino acid substitutions thereof, and the light chain variable region (V_{L}) comprises a sequence selected from the group consisting of SEQ ID NO:438, SEQ ID NO:440, and conservative amino acid substitutions thereof.

In an embodiment, an anti-LFA-1 or anti-CD11a scFv domain comprises the heavy chain CDR1, CDR2 and CDR3 domains having the sequences set forth in SEQ ID NO:441, SEQ ID NO:442, and/or SEQ ID NO:443, respectively, or conservative amino acid substitutions thereof, and/or light chain CDR1, CDR2 and CDR3 domains having the sequences set forth in SEQ ID NO:444, SEQ ID NO:445, and/or SEQ ID NO:446, respectively, or conservative amino acid substitutions thereof.

In an embodiment, the anti-LFA-1 binding domain or anti-CD11a binding domain includes scFv, V_{H} and/or V_{L} sequences or a heavy chain and/or a light chain CDR1, CDR2, and/or CDR3 sequence of odulimomab, or derivatives, variants, or fragments thereof, or humanized variants thereof. Odulimomab is commercially available from Creative Biolabs, Inc. (Shirley, NY, USA).

In an embodiment, an anti-LFA-1 binding domain or anti-CD11a binding domain, including additional scFv, V_{H} and/or V_{L} sequences or a heavy chain and/or a light chain CDR1, CDR2, and/or CDR3 sequence, is obtained from the antibodies disclosed in U.S. Pat. No. 5,284,931, the disclosures of which are incorporated by reference herein. In an embodiment, an anti-LFA-1 binding domain, including additional scFv, V_{H} and/or V_{L} sequences or a heavy chain and/or a light chain CDR1, CDR2, and/or CDR3 sequence, is obtained from the antibodies produced by cell lines deposited with the ATCC, including M17/4.4 (ATCC TIB-217), TS2/18.1.1 (ATCC HB-195), TS1/22.1.1.13 (ATCC HB-202), TS1/18.1.2.11 (ATCC HB-203), LM2/1.6.11 (ATCC HB-204), TS2/9.1.4.3 (ATCC HB-205), 2E6 (ATCC HB-226), BE29G1 (ATCC HB-233), TS2/16.2.1 (ATCC HB-243), TS2/4.1.1 (ATCC HB-244), TS2/7.1.1 (ATCC HB-245), S6F1 (ATCC HB-9579), M5/114.15.2 (ATCC IIB-120), M1/70.15.11.5 HL (ATCC TIB-128), FD441.8 (ATCC TIB-213), M17/4.4.11.9 (ATCC TIB-217), M18/2.a.12.7 (ATCC TIB-218), M17/5.2 (ATCC TIB-237), and M5/49.4.1 (ATCC TIB-238).

In an embodiment, the anti-LFA-1 binding domain or anti-CD11a binding domain includes additional antibodies, including scFv, V_{H} and/or V_{L} sequences or a heavy chain and/or a light chain CDR1, CDR2, and/or CDR3 sequence as disclosed in U.S. Patent Application Publication No. US 2008/0038259 A1, the disclosures of which are incorporated by reference herein.

### 9. Extracellular FAP Binding Domains

In an embodiment, a CCR comprises an extracellular domain, wherein the extracellular domain is a domain capable of binding to human FAP. In an embodiment, the extracellular domain binds to human FAP, also known as fibroblast activation protein and fibroblast activating protein a. The function of FAP and its role in tumor stroma in solid tumors, including desmoplasia, and its expression on cancer-associated fibroblasts found in breast, lung, colon, pancreatic, and other tumors, are described in Liu, et al., Cancer Biol. & Ther. 2012, 13, 123-129, the disclosure of which is incorporated by reference herein. In an embodiment, the extracellular FAP binding domain is a scFv domain. In an embodiment, the FAP scFv binding domain binds to murine FAP. In an embodiment, the FAP scFv binding domain binds to human FAP. In an embodiment, a CCR of the present invention comprises a construct as shown in FIG. 34, wherein the V_{H} and V_{L} domains are anti-FAP V_{H} and V_{L} domains, and the linker is as described herein.

In an embodiment, a CCR of the present invention comprises an extracellular domain, wherein the extracellular domain comprises a FAP binding domain. In an embodiment, the FAP binding domain is an anti-FAP binding domain described in U.S. Patent Application Publication No. US 2019/0233536 A1, the disclosures of which is incorporated by reference herein. In an embodiment, the anti-FAP binding domain includes scFv, V_{H} and/or V_{L} sequences or heavy chain and/or light chain CDR1, CDR2, and/or CDR3 sequences of sibrotuzumab, also known as BIBH1, which are described in U.S. Patent Application Publication No. US 2003/0103968 A1, the disclosures of which are incorporated by reference herein. In an embodiment, the anti-FAP binding domain includes scFv, V_{H} and/or V_{L} sequences or heavy chain and/or light chain CDR1, CDR2, and/or CDR3 sequences of FAP5, which are described in U.S. Patent Application Publication No. US 2009/0304718 A1, the disclosures of which are incorporated by reference herein. The amino acid sequences of exemplary FAP binding scFv domains are provided in Table 51.

TABLE 51
Amino acid sequences of exemplary FAP binding scFv domains.

| Identifier | Sequence (One-Letter Amino Acid Symbols) | |
|---|---|---|
| SEQ ID NO: 447 | DIVMTQSPDS LAVSLGERAT INCKSSQSLL YSRNQKNYLA WYQQKPGQPP KLLIFWASTR | 60 |
| Anti-FAP scFv | ESGVPDRFSG SGFGTDFTLT ISSLQAEDVA VYYCQQYFSY PLTFGQGTKV EIKGGGGSGG | 120 |
| | GGSGGGGSQV QLVQSGAEVK KPGASVKVSC KTSRYTFTEY TIHWVRQAPG QRLEWIGGIN | 180 |
| | PNNGIPNYNQ KFKGRVTITV DTSASTAYME LSSLRSEDTA VYYCARRRIA YGYDEGHAMD | 240 |
| | YWGQGTLVTV SS | 252 |
| SEQ ID NO: 448 | QVQLVQSGAE VKKPGASVKV SCKTSRYTFT EYTIEWVRQA PGQRLEWIGG INPNNGIPNY | 60 |
| Anti-FAP | NQKFKGRVTI TVDTSASTAY MELSSLRSED TAVYYCARRR IAYGYDEGHA MDYWGQGTLV | 120 |
| sibrotuzumab | TVSS | 124 |
| variable heavy | | |
| chain | | |
| SEQ ID NO: 449 | DIVMTQSPDS LAVSLGERAT INCKSSQSLL YSRNQKNYLA WYQQKPGQPP KLLIFWASTR | 60 |
| Anti-FAP | ESGVPDRFSG SGFGTDFTLT ISSLQAEDVA VYYCQQYFSY PLTFGQGTKV EIK | 113 |
| sibrotuzumab | | |
| variable light | | |
| chain | | |
| SEQ ID NO: 450 | QVQLQQSGAE LARPGASVNL SCKASGYTFT NNGINWLKQR TGQGLEWIGE IYPRSTNTLY | 60 |
| Anti-FAP FAP5 | NEKFKGKATL TADRSSNTAY MELRSLTSED SAVYFCARTL TAPFAFWGQG TLVTVSA | 117 |
| variable heavy | | |
| chain | | |
| SEQ ID NO: 451 | QIVLTQSPAI MSASPGEKVT MTCSASSGVN FMHWYQQKSG TSPKRWIFDT SKLASGVPAR | 60 |
| Anti-FAP FAP5 | FSGSGSGTSY SLTISSMEAE DAATYYCQQW SFNPPTFGGG TKLEIKR | 107 |
| variable light | | |
| chain | | |

In an embodiment, an anti-FAP scFv domain comprises the sequence shown in SEQ ID NO:447, or conservative amino acid substitutions thereof. In an embodiment, an anti-FAP scFv domain comprises a scFv domain that is at least 99% identical to the sequence shown in SEQ ID NO:447. In an embodiment, an anti-FAP scFv domain comprises a scFv domain that is at least 98% identical to the sequence shown in SEQ ID NO:447. In an embodiment, an anti-FAP scFv domain comprises a scFv domain that is at least 97% identical to the sequence shown in SEQ ID NO:447. In an embodiment, an anti-FAP scFv domain comprises a scFv domain that is at least 96% identical to the sequence shown in SEQ ID NO:447. In an embodiment, an anti-FAP scFv domain comprises a scFv domain that is at least 95% identical to the sequence shown in SEQ ID NO:447. In an embodiment, an anti-FAP scFv domain comprises a scFv domain that is at least 90% identical to the sequence shown in SEQ ID NO:447. In an embodiment, an anti-FAP scFv domain comprises a scFv domain that is at least 85% identical to the sequence shown in SEQ ID NO:447. In an embodiment, an anti-FAP scFv domain comprises a scFv domain that is at least 80% identical to the sequence shown in SEQ ID NO:447.

In an embodiment, an anti-FAP scFv domain comprises a heavy chain variable region (V_{H}) domain and/or a light chain variable region (V_{L}) domain, wherein the V_{H} domain comprises the sequence shown in SEQ ID NO:448, or conservative amino acid substitutions thereof, and the light chain variable region (V_{L}) comprises the sequence shown in SEQ ID NO:449, or conservative amino acid substitutions thereof. In an embodiment, an anti-FAP scFv domain comprises V_{H} and V_{L} regions that are each at least 99% identical to the sequences shown in SEQ ID NO:448 and SEQ ID NO:449, respectively. In an embodiment, an anti-FAP scFv domain comprises V_{H} and V_{L} regions that are each at least 98% identical to the sequences shown in SEQ ID NO:448 and SEQ ID NO:449, respectively. In an embodiment, an anti-FAP scFv domain comprises V_{H} and V_{L} regions that are each at least 97% identical to the sequences shown in SEQ ID NO:448 and SEQ ID NO:449, respectively. In an embodiment, an anti-FAP scFv domain comprises V_{H} and V_{L} regions that are each at least 96% identical to the sequences shown in SEQ ID NO:448 and SEQ ID NO:449, respectively. In an embodiment, an anti-FAP scFv domain comprises V_{H} and V_{L} regions that are each at least 95% identical to the sequences shown in SEQ ID NO:448 and SEQ ID NO:449, respectively. In an embodiment, an anti-FAP scFv domain comprises V_{H} and V_{L} regions that are each at least 90% identical to the sequences shown in SEQ ID NO:448 and SEQ ID NO:449, respectively. In an embodiment, an anti-FAP scFv domain comprises V_{H} and V_{L} regions that are each at least 85% identical to the sequences shown in SEQ ID NO:448 and SEQ ID NO:449, respectively. In an embodiment, an anti-FAP scFv domain comprises V_{H} and V_{L} regions that are each at least 80% identical to the sequences shown in SEQ ID NO:448 and SEQ ID NO:449, respectively.

In an embodiment, an anti-FAP scFv domain comprises a heavy chain variable region (V_{H}) domain and/or a light chain variable region (V_{L}) domain, wherein the V_{H} domain comprises the sequence shown in SEQ ID NO:450, or conservative amino acid substitutions thereof, and the light chain variable region (V_{L}) comprises the sequence shown in SEQ ID NO:451, or conservative amino acid substitutions thereof. In an embodiment, an anti-FAP scFv domain comprises V_{H} and V_{L} regions that are each at least 99% identical to the sequences shown in SEQ ID NO:450 and SEQ ID NO:451, respectively. In an embodiment, an anti-FAP scFv domain comprises V_{H} and V_{L} regions that are each at least 98% identical to the sequences shown in SEQ ID NO:450 and SEQ ID NO:451, respectively. In an embodiment, an anti-FAP scFv domain comprises V_{H} and V_{L} regions that are each at least 97% identical to the sequences shown in SEQ ID NO:450 and SEQ ID NO:451, respectively. In an embodiment, an anti-FAP scFv domain comprises V_{H} and V_{L} regions that are each at least 96% identical to the sequences shown in SEQ ID NO:450 and SEQ ID NO:451, respectively. In an embodiment, an anti-FAP scFv domain comprises VII and V_{L} regions that are each at least 95% identical to the sequences shown in SEQ ID NO:450 and SEQ ID NO:451, respectively. In an embodiment, an anti-FAP scFv domain comprises V_{H} and V_{L} regions that are each at least 90% identical to the sequences shown in SEQ ID NO:450 and SEQ ID NO:451, respectively. In an embodiment, an anti-FAP scFv domain comprises V_{H} and V_{L} regions that are each at least 85% identical to the sequences shown in SEQ ID NO:450 and SEQ ID NO:451, respectively. In an embodiment, an anti-FAP scFv domain comprises V_{H} and V_{L} regions that are each at least 80% identical to the sequences shown in SEQ ID NO:450 and SEQ ID NO:451, respectively.

In an embodiment, an anti-FAP scFv domain comprises a V_{H} domain and a V_{L} domain, wherein the V_{H} domain comprises a sequence selected from the group consisting of SEQ ID NO:448, SEQ ID NO:450, and conservative amino acid substitutions thereof, and the light chain variable region (V_{L}) comprises a sequence selected from the group consisting of SEQ ID NO:449, SEQ ID NO:451, and conservative amino acid substitutions thereof.

In an embodiment, the anti-FAP binding domain includes scFv, V_{H} and/or V_{L} sequences or heavy chain and/or light chain CDR1, CDR2, and/or CDR3 sequences of OMTX-705 (available from Oncomatryx SL), which are described in U.S. Pat. No. 10,864,278, the disclosures of which are incorporated by reference herein.

The nucleotide sequences encoding exemplary FAP binding V_{H} and V_{L} domains and CDR domains for scFv domains are provided in Table 52 and are further described in U.S. Patent Application Publication Nos. US 2003/0103968 A1; US 2009/0304718 A1; and US 2019/0233536 A1; the disclosures of which are incorporated by reference herein. In an embodiment, a nucleotide sequence in Table 52 is codon-optimized to improve protein expression.

TABLE 52
Nucleotide sequences of exemplary FAP binding scFv domains.

| Identifier | Sequence (One-Letter Nucleotide Symbols) | |
|---|---|---|
| SEQ ID NO: 452 | CAGGTGCAAC TAGTGCAGTC CGGCGCCGAA GTGAAGAAAC CCGGTGCTTC CGTGAAAGTC | 60 |
| Anti-FAP | AGCTGTAAAA CTAGTAGATA CACCTTCACT GAATACACCA TACACTGGGT TAGACAGGCC | 120 |
| sibrotuzumab | CCTGGCCAAA GGCTGGAGTG GATAGGAGGT ATTAATCCTA ACAATGGTAT TCCTAACTAC | 180 |
| variable heavy | AACCAGAAGT TCAAGGGCCG GGTCACCATC ACCGTAGACA CCTCTGCCAG CACCGCCTAC | 240 |
| chain | ATGGAACTGT CCAGCCTGCG CTCCGAGGAC ACTGCAGTCT ACTACTGCGC CAGAAGAAGA | 300 |
| | ATCGCCTATG GTTACGACGA GGGCCATGCT ATGGACTACT GGGGTCAAGG AACCCTTGTC | 360 |
| | ACCGTCTCCT CA | 372 |
| SEQ ID NO: 453 | GACATTGTGA TGACCCAATC TCCAGACTCT TTGGCTGTGT CTCTAGGGGA GAGGGCCACC | 60 |
| Anti-FAP | ATCAACTGCA AGTCCAGTCA GAGCCTTTTA TATTCTAGAA ATCAAAAGAA CTACTTGGCC | 120 |
| sibrotuzumab | TGGTATCAGC AGAAACCAGG ACAGCCACCC AAACTCCTCA TCTTTTGGGC TAGCACTAGG | 180 |
| variable light | GAATCTGGGG TACCTGATAG GTTCAGTGGC AGTGGGTTTG GGACAGACTT CACCCTCACC | 240 |
| chain | ATTAGCAGCC TGCAGGCTGA AGATGTGGCA GTTTATTACT GTCAGCAATA TTTTAGCTAT | 300 |
| | CCGCTCACGT TCGGACAAGG GACCAAGGTG GAAATAAAA | 339 |

In an embodiment, an anti-FAP scFv domain comprises a heavy chain variable region (V_{H}) domain and/or a light chain variable region (V_{L}) domain of the antibody sibrotuzumab encoded by a nucleotide sequence. In an embodiment, an anti-TF scFv domain comprises a V_{H} domain and/or a V_{L} domain, wherein the V_{H} domain is encoded by the sequence shown in SEQ ID NO:452 and the light chain variable region (V_{L}) is encoded by the sequence shown in SEQ ID NO:453. In an embodiment, an anti-TF scFv domain comprises V_{H} and/or V_{L} regions that are encoded by nucleotides that are each at least 99% identical to the sequences shown in SEQ ID NO:452 and SEQ ID NO:453, respectively. In an embodiment, an anti-TF scFv domain comprises V_{H} and/or V_{L} regions that are encoded by nucleotides that are each at least 98% identical to the sequences shown in SEQ ID NO:452 and SEQ ID NO:453, respectively. In an embodiment, an anti-TF scFv domain comprises V_{H} and/or V_{L} regions that are encoded by nucleotides that are each at least 97% identical to the sequences shown in SEQ ID NO:452 and SEQ ID NO:453, respectively. In an embodiment, an anti-TF scFv domain comprises V_{H} and/or V_{L} regions that are encoded by nucleotides that are each at least 96% identical to the sequences shown in SEQ ID NO:452 and SEQ ID NO:453, respectively. In an embodiment, an anti-TF scFv domain comprises V_{H} and/or V_{L} regions that are encoded by nucleotides that are each at least 95% identical to the sequences shown in SEQ ID NO:452 and SEQ ID NO:453, respectively. In an embodiment, an anti-TF scFv domain comprises V_{H} and/or V_{L} regions that are encoded by nucleotides that are each at least 90% identical to the sequences shown in SEQ ID NO:452 and SEQ ID NO:453, respectively. In an embodiment, an anti-TF scFv domain comprises V_{H} and/or V_{L} regions that are encoded by nucleotides that are each at least 85% identical to the sequences shown in SEQ ID NO:452 and SEQ ID NO:453, respectively. In an embodiment, an anti-TF scFv domain comprises V_{H} and/or V_{L} regions that are encoded by nucleotides that are each at least 80% identical to the sequences shown in SEQ ID NO:452 and SEQ ID NO:453, respectively. In an embodiment, including the foregoing embodiments, SEQ ID NO:452 and/or SEQ ID NO:453 is codon-optimized to improve protein expression.

### 10. Extracellular VISTA Binding Domains

In an embodiment, a CCR comprises an extracellular domain, wherein the extracellular domain is a domain capable of binding to V-domain-containing Ig suppressor of T-cell activation (VISTA), also known as c10orf54, PD-1H and B7-H5, which is an immune checkpoint gene that, without being bound by theory, is believed to inhibit anti-tumor immune responses. VISTA and its properties have been described in Wang, et al., J. Exp. Med. 2011, 208, 577-92; Nowak, et al., Immunol. Rev. 2017, 276, 66-79; and Deng, et al., J. Immunother. Cancer, 2016, 4, 86; the disclosures of each of which are incorporated by reference herein. VISTA is expressed on myeloid cells and T-lymphocytes and its overexpression is associated with suppression of early T-cell activation and proliferation and reduction of cytokine production. VISTA acts as both a ligand on antigen-presenting cells and as a receptor on T-cells. VISTA is also known to be overexpressed on certain lung cancers, such as mesothelioma and pleural mesothelioma. In an embodiment, the CCR comprises an extracellular domain that binds to human VISTA. In an embodiment, the extracellular domain binds to murine VISTA. In an embodiment, the extracellular VISTA binding domain is a scFv domain. In an embodiment, a CCR of the present invention comprises a construct as shown in FIG. 34, wherein the V_{H} and V_{L} domains are anti-VISTA V_{H} and V_{L} domains, and the linker is as described herein.

In an embodiment, the scFv domain includes a scFv, V_{H}, V_{L}, or CDR domain of the antibodies 1B8, 2C12, 1A12, or 3C5, the preparation and properties of each of which are described in U.S. Patent Application Publication No. US 2020/0407449 A1 and are incorporated by reference herein, including the V_{H}, V_{L}, and CDR domains of each of 1B8, 2C12, 1A12, or 3C5, and variants, derivatives, and fragments thereof. In an embodiment, the VISTA scFv domain of a CCR of the present invention includes scFv antibodies comprising the V_{H} and V_{L} domains described in U.S. Patent Application Publication No. US 2020/0407449 A1, the disclosures of which are incorporated by reference herein. The amino acid sequences of exemplary VISTA binding V_{H} and V_{L} domains are provided in Table 53.

TABLE 53
Amino acid sequences of exemplary VISTA binding scFv domains.

| Identifier | Sequence (One-Letter Amino Acid Symbols) | |
|---|---|---|
| SEQ ID NO: 454 | QMQLVQSGAE VKKPGSSVKV SCKASGGSSS NYAISWVRQA PGQGLEWMGG IIPIFGTTSY | 60 |
| Anti-VISTA | AQKFQGRVTI TADGSMSTAY MELSSLRSED TAVYYCAKGA IEPWPFYFDN WGQGTLVTVS | 120 |
| antibody 1B8 | S | 121 |
| variable heavy | | |
| chain | | |
| SEQ ID NO: 455 | QLVLTQPRSV SGSPGQSVTI SCTGTNSDVG AYNYLSWYQQ LPGRAPKVII YDVNKRPSGV | 60 |
| Anti-VISTA | PDRFSGSRSG KTASLTISGL QAEDEADYYC AAWDDSLNGL VFGGGTKLTV LG | 112 |
| antibody 1B8 | | |
| variable heavy | | |
| chain | | |
| SEQ ID NO: 456 | GGSSSNYA | 8 |
| Anti-VISTA | | |
| antibody 1B8 | | |
| variable heavy | | |
| chain CDR1 | | |
| SEQ ID NO: 457 | IIPIFGTT | 8 |
| Anti-VISTA | | |
| antibody 1B8 | | |
| variable heavy | | |
| chain CDR2 | | |
| SEQ ID NO: 458 | AKGAIEPWPF YFDN | 14 |
| Anti-VISTA | | |
| antibody 1B8 | | |
| variable heavy | | |
| chain CDR3 | | |
| SEQ ID NO: 459 | NSDVGAYNY | 9 |
| Anti-VISTA | | |
| antibody 1B8 | | |
| variable light | | |
| chain CDR1 | | |
| SEQ ID NO: 460 | DVN | 3 |
| Anti-VISTA | | |
| antibody 1B8 | | |
| variable light | | |
| chain CDR2 | | |
| SEQ ID NO: 461 | AAWDDSLNGL V | 11 |
| Anti-VISTA | | |
| antibody 1B8 | | |
| variable light | | |
| chain CDR3 | | |
| SEQ ID NO: 462 | QVQLVESGAE VKKPGASVKV SCKASGYIFT DYYMEWVRQA PGQGLEWMGV INPYDGRTSF | 60 |
| Anti-VISTA | AQKFQGRLTV TRDTSTSTAY MDLSGLRSED TAVYYCAKQM GIWDYDAFDI WGQGTMVTVS | 120 |
| antibody 2C12 | S | 121 |
| variable heavy | | |
| chain | | |
| SEQ ID NO: 463 | QFVLTQPSSV SGAPGQRVII SCTGSSSNIG AGYDVHWYQQ LPGTAPKVLI YGNSDRPSGV | 60 |
| Anti-VISTA | PDRFSASKSA TSASLAITGL QAEDEADYYC VAWDDSLKAY VFGTGTKVTV LG | 112 |
| antibody 2C12 | | |
| variable heavy | | |
| chain | | |
| SEQ ID NO: 464 | GYIFTDYY | 8 |
| Anti-VISTA | | |
| antibody 2C12 | | |
| variable heavy | | |
| chain CDR1 | | |
| SEQ ID NO: 465 | INPYDGRT | 8 |
| Anti-VISTA | | |
| antibody 2C12 | | |
| variable heavy | | |
| chain CDR2 | | |
| SEQ ID NO: 466 | AKQMGIWDYD AFDI | 14 |
| Anti-VISTA | | |
| antibody 2C12 | | |
| variable heavy | | |
| chain CDR3 | | |
| SEQ ID NO: 467 | SSNIGAGYD | 9 |
| Anti-VISTA | | |
| antibody 2C12 | | |
| variable light | | |
| chain CDR1 | | |
| SEQ ID NO: 468 | GNS | 3 |
| Anti-VISTA | | |
| antibody 2C12 | | |
| variable light | | |
| chain CDR2 | | |
| SEQ ID NO: 469 | VAWDDSLKAY V | 11 |
| Anti-VISTA | | |
| antibody 2C12 | | |
| variable light | | |
| chain CDR3 | | |
| SEQ ID NO: 470 | QMQLVESGGG LVEPGRSLRL SCTTSGFSFH DYTMYWVRQV PGKGLEWVSL ISWDGTITFY | 60 |
| Anti-VISTA | ADPVRGRFTI SRDNSKNSLY LQMNSLRAED TAVYYCAKED RYDYYSGAFD IWGQGTVVTV | 120 |
| antibody 1A12 | SS | 122 |
| variable heavy | | |
| chain | | |
| SEQ ID NO: 471 | QSALTQPRSV SGSPGQSVTI SCTGTSSDVG GYDYVSWYQQ HPGKAPKLIL NDVNKRPSGV | 60 |
| Anti-VISTA | PDRFSGSKSG NTASLTISGL QPDDEADYYC SSFAGSNTLR VFGGGTKLTV LG | 112 |
| antibody 1A12 | | |
| variable heavy | | |
| chain | | |
| SEQ ID NO: 472 | GFSFHDYT | 8 |
| Anti-VISTA | | |
| antibody 1A12 | | |
| variable heavy | | |
| chain CDR1 | | |
| SEQ ID NO: 473 | ISWDGTIT | 8 |
| Anti-VISTA | | |
| antibody 1A12 | | |
| variable heavy | | |
| chain CDR2 | | |
| SEQ ID NO: 474 | AKEDRYDYYS GAFDI | 15 |
| Anti-VISTA | | |
| antibody 1A12 | | |
| variable heavy | | |
| chain CDR3 | | |
| SEQ ID NO: 475 | SSDVGGYDY | 9 |
| Anti-VISTA | | |
| antibody 1A12 | | |
| variable light | | |
| chain CDR1 | | |
| SEQ ID NO: 476 | DVN | 3 |
| Anti-VISTA | | |
| antibody 1A12 | | |
| variable light | | |
| chain CDR2 | | |
| SEQ ID NO: 477 | SSFAGSNTLR V | 11 |
| Anti-VISTA | | |
| antibody 1A12 | | |
| variable light | | |
| chain CDR3 | | |
| SEQ ID NO: 478 | QVQLVQSGAE VKKPGASVKL SCKASGYTFS SYWMEWVRQA PGQRLEWMGE INPGNGHTNY | 60 |
| Anti-VISTA | NEKFKSRVTI TVDKSASTAY MELSSLRSED TAVYYCAKDI AYYDFWSGDA FDLWGQGTMV | 120 |
| antibody 3C5 | TVSS | 124 |
| variable heavy | | |
| chain | | |
| SEQ ID NO: 479 | SYELTQPLSV SVSPGQTASI TCSGDKLGNK YASWYQQKPG QSPVLVIYQD NKRPSGIPER | 60 |
| Anti-VISTA | FSGSNSGNTA TLTISGTQAT DEADYYCQTW DRSTGVFGTG TKVTVLG | 107 |
| antibody 3C5 | | |
| variable heavy | | |
| chain | | |
| SEQ ID NO: 480 | GYTFSSYW | 8 |
| Anti-VISTA | | |
| antibody 3C5 | | |
| variable heavy | | |
| chain CDR1 | | |
| SEQ ID NO: 481 | INPGNGET | 8 |
| Anti-VISTA | | |
| antibody 3C5 | | |
| variable heavy | | |
| chain CDR2 | | |
| SEQ ID NO: 482 | AKDIAYYDFW SGDAFDL | 17 |
| Anti-VISTA | | |
| antibody 3C5 | | |
| variable heavy | | |
| chain CDR3 | | |
| SEQ ID NO: 483 | KLGNKY | 6 |
| Anti-VISTA | | |
| antibody 3C5 | | |
| variable light | | |
| chain CDR1 | | |
| SEQ ID NO: 484 | QDN | 3 |
| Anti-VISTA | | |
| antibody 3C5 | | |
| variable light | | |
| chain CDR2 | | |
| SEQ ID NO: 485 | QTWDRSTGV | 9 |
| Anti-VISTA | | |
| antibody 3C5 | | |
| variable light | | |
| chain CDR3 | | |

In an embodiment, an anti-VISTA scFv domain comprises a heavy chain variable region (V_{H}) domain and/or a light chain variable region (V_{L}) domain of the antibody 1B8, or variants, derivatives, fragments, or conservative amino acid substitutions thereof. In an embodiment, an anti-VISTA scFv domain comprises a V_{H} domain and a V_{L} domain, wherein the V_{H} domain comprises the sequence shown in SEQ ID NO:454, or conservative amino acid substitutions thereof, and the light chain variable region (V_{L}) comprises the sequence shown in SEQ ID NO:455, or conservative amino acid substitutions thereof. In an embodiment, an anti-VISTA scFv domain comprises V_{H} and V_{L} regions that are each at least 99% identical to the sequences shown in SEQ ID NO:454 and SEQ ID NO:455, respectively. In an embodiment, an anti-VISTA scFv domain comprises V_{H} and V_{L} regions that are each at least 98% identical to the sequences shown in SEQ ID NO:454 and SEQ ID NO:455, respectively. In an embodiment, an anti-VISTA scFv domain comprises V_{H} and V_{L} regions that are each at least 97% identical to the sequences shown in SEQ ID NO:454 and SEQ ID NO:455, respectively. In an embodiment, an anti-VISTA scFv domain comprises V_{H} and V_{L} regions that are each at least 96% identical to the sequences shown in SEQ ID NO:454 and SEQ ID NO:455, respectively. In an embodiment, an anti-VISTA scFv domain comprises V_{H} and V_{L} regions that are each at least 95% identical to the sequences shown in SEQ ID NO:454 and SEQ ID NO:455, respectively. In an embodiment, an anti-VISTA scFv domain comprises V_{H} and V_{L} regions that are each at least 90% identical to the sequences shown in SEQ ID NO:454 and SEQ ID NO:455, respectively. In an embodiment, an anti-VISTA scFv domain comprises V_{H} and V_{L} regions that are each at least 85% identical to the sequences shown in SEQ ID NO:454 and SEQ ID NO:455, respectively. In an embodiment, an anti-VISTA scFv domain comprises V_{H} and V_{L} regions that are each at least 80% identical to the sequences shown in SEQ ID NO:454 and SEQ ID NO:455, respectively.

In an embodiment, an anti-VISTA scFv domain comprises the heavy chain CDR1, CDR2 and CDR3 domains and the light chain CDR1, CDR2, and CDR3 domains, or conservative amino acid substitutions thereof, of the antibody 1B1. In an embodiment, an anti-VISTA scFv domain comprises the heavy chain CDR1, CDR2 and CDR3 domains of having the sequences set forth in SEQ ID NO:456, SEQ ID NO:457, and/or SEQ ID NO:458, respectively, or conservative amino acid substitutions thereof, and/or light chain CDR1, CDR2 and CDR3 domains having the sequences set forth in SEQ ID NO:459, SEQ ID NO:460, and/or SEQ ID NO:461, respectively, or conservative amino acid substitutions thereof.

In an embodiment, an anti-VISTA scFv domain comprises a heavy chain variable region (V_{H}) domain and/or a light chain variable region (V_{L}) domain of the antibody 2C12, or variants, derivatives, fragments, or conservative amino acid substitutions thereof. In an embodiment, an anti-VISTA scFv domain comprises a V_{H} domain and a V_{L} domain, wherein the V_{H} domain comprises the sequence shown in SEQ ID NO:462, or conservative amino acid substitutions thereof, and the light chain variable region (V_{L}) comprises the sequence shown in SEQ ID NO:463, or conservative amino acid substitutions thereof. In an embodiment, an anti-VISTA scFv domain comprises V_{H} and V_{L} regions that are each at least 99% identical to the sequences shown in SEQ ID NO:462 and SEQ ID NO:463, respectively. In an embodiment, an anti-VISTA scFv domain comprises V_{H} and V_{L} regions that are each at least 98% identical to the sequences shown in SEQ ID NO:462 and SEQ ID NO:463, respectively. In an embodiment, an anti-VISTA scFv domain comprises V_{H} and V_{L} regions that are each at least 97% identical to the sequences shown in SEQ ID NO:462 and SEQ ID NO:463, respectively. In an embodiment, an anti-VISTA scFv domain comprises V_{H} and V_{L} regions that are each at least 96% identical to the sequences shown in SEQ ID NO:462 and SEQ ID NO:463, respectively. In an embodiment, an anti-VISTA scFv domain comprises V_{H} and V_{L} regions that are each at least 95% identical to the sequences shown in SEQ ID NO:462 and SEQ ID NO:463, respectively. In an embodiment, an anti-VISTA scFv domain comprises V_{H} and V_{L} regions that are each at least 90% identical to the sequences shown in SEQ ID NO:462 and SEQ ID NO:463, respectively. In an embodiment, an anti-VISTA scFv domain comprises V_{H} and V_{L} regions that are each at least 85% identical to the sequences shown in SEQ ID NO:462 and SEQ ID NO:463, respectively. In an embodiment, an anti-VISTA scFv domain comprises V_{H} and V_{L} regions that are each at least 80% identical to the sequences shown in SEQ ID NO:462 and SEQ ID NO:463, respectively.

In an embodiment, an anti-VISTA scFv domain comprises the heavy chain CDR1, CDR2 and CDR3 domains and the light chain CDR1, CDR2, and CDR3 domains, or conservative amino acid substitutions thereof, of the antibody 2C12. In an embodiment, an anti-VISTA scFv domain comprises the heavy chain CDR1, CDR2 and CDR3 domains of having the sequences set forth in SEQ ID NO:464, SEQ ID NO:465, and/or SEQ ID NO:466, respectively, or conservative amino acid substitutions thereof, and/or light chain CDR1, CDR2 and CDR3 domains having the sequences set forth in SEQ ID NO:467, SEQ ID NO:468, and/or SEQ ID NO:469, respectively, or conservative amino acid substitutions thereof.

In an embodiment, an anti-VISTA scFv domain comprises a heavy chain variable region (V_{H}) domain and/or a light chain variable region (V_{L}) domain of the antibody 1A12, or variants, derivatives, fragments, or conservative amino acid substitutions thereof. In an embodiment, an anti-VISTA scFv domain comprises a V_{H} domain and a V_{L} domain, wherein the V_{H} domain comprises the sequence shown in SEQ ID NO:470, or conservative amino acid substitutions thereof, and the light chain variable region (V_{L}) comprises the sequence shown in SEQ ID NO:471, or conservative amino acid substitutions thereof. In an embodiment, an anti-VISTA scFv domain comprises V_{H} and V_{L} regions that are each at least 99% identical to the sequences shown in SEQ ID NO:470 and SEQ ID NO:471, respectively. In an embodiment, an anti-VISTA scFv domain comprises V_{H} and V_{L} regions that are each at least 98% identical to the sequences shown in SEQ ID NO:470 and SEQ ID NO:471, respectively. In an embodiment, an anti-VISTA scFv domain comprises V_{H} and V_{L} regions that are each at least 97% identical to the sequences shown in SEQ ID NO:470 and SEQ ID NO:471, respectively. In an embodiment, an anti-VISTA scFv domain comprises V_{H} and V_{L} regions that are each at least 96% identical to the sequences shown in SEQ ID NO:470 and SEQ ID NO:471, respectively. In an embodiment, an anti-VISTA scFv domain comprises V_{H} and V_{L} regions that are each at least 95% identical to the sequences shown in SEQ ID NO:470 and SEQ ID NO:471, respectively. In an embodiment, an anti-VISTA scFv domain comprises V_{H} and V_{L} regions that are each at least 90% identical to the sequences shown in SEQ ID NO:470 and SEQ ID NO:471, respectively. In an embodiment, an anti-VISTA scFv domain comprises V_{H} and V_{L} regions that are each at least 85% identical to the sequences shown in SEQ ID NO:470 and SEQ ID NO:471, respectively. In an embodiment, an anti-VISTA scFv domain comprises V_{H} and V_{L} regions that are each at least 80% identical to the sequences shown in SEQ ID NO:470 and SEQ ID NO:471, respectively.

In an embodiment, an anti-VISTA scFv domain comprises the heavy chain CDR1, CDR2 and CDR3 domains and the light chain CDR1, CDR2, and CDR3 domains, or conservative amino acid substitutions thereof, of the antibody 2C12. In an embodiment, an anti-VISTA scFv domain comprises the heavy chain CDR1, CDR2 and CDR3 domains of having the sequences set forth in SEQ ID NO:472, SEQ ID NO:473, and/or SEQ ID NO:474, respectively, or conservative amino acid substitutions thereof, and/or light chain CDR1, CDR2 and CDR3 domains having the sequences set forth in SEQ ID NO:475, SEQ ID NO:476, and/or SEQ ID NO:477, respectively, or conservative amino acid substitutions thereof.

In an embodiment, an anti-VISTA scFv domain comprises a heavy chain variable region (V_{H}) domain and/or a light chain variable region (V_{L}) domain of the antibody 3C5, or variants, derivatives, fragments, or conservative amino acid substitutions thereof. In an embodiment, an anti-VISTA scFv domain comprises a V_{H} domain and a V_{L} domain, wherein the V_{H} domain comprises the sequence shown in SEQ ID NO:478, or conservative amino acid substitutions thereof, and the light chain variable region (V_{L}) comprises the sequence shown in SEQ ID NO:479, or conservative amino acid substitutions thereof. In an embodiment, an anti-VISTA scFv domain comprises V_{H} and V_{L} regions that are each at least 99% identical to the sequences shown in SEQ ID NO:478 and SEQ ID NO:479, respectively. In an embodiment, an anti-VISTA scFv domain comprises V_{H} and V_{L} regions that are each at least 98% identical to the sequences shown in SEQ ID NO:478 and SEQ ID NO:479, respectively. In an embodiment, an anti-VISTA scFv domain comprises V_{H} and V_{L} regions that are each at least 97% identical to the sequences shown in SEQ ID NO:478 and SEQ ID NO:479, respectively. In an embodiment, an anti-VISTA scFv domain comprises V_{H} and V_{L} regions that are each at least 96% identical to the sequences shown in SEQ ID NO:478 and SEQ ID NO:479, respectively. In an embodiment, an anti-VISTA scFv domain comprises V_{H} and V_{L} regions that are each at least 95% identical to the sequences shown in SEQ ID NO:478 and SEQ ID NO:479, respectively. In an embodiment, an anti-VISTA scFv domain comprises V_{H} and V_{L} regions that are each at least 90% identical to the sequences shown in SEQ ID NO:478 and SEQ ID NO:479, respectively. In an embodiment, an anti-VISTA scFv domain comprises V_{H} and V_{L} regions that are each at least 85% identical to the sequences shown in SEQ ID NO:478 and SEQ ID NO:479, respectively. In an embodiment, an anti-VISTA scFv domain comprises V_{H} and V_{L} regions that are each at least 80% identical to the sequences shown in SEQ ID NO:478 and SEQ ID NO:479, respectively.

In an embodiment, an anti-VISTA scFv domain comprises the heavy chain CDR1, CDR2 and CDR3 domains and the light chain CDR1, CDR2, and CDR3 domains, or conservative amino acid substitutions thereof, of the antibody 3C5. In an embodiment, an anti-VISTA scFv domain comprises the heavy chain CDR1, CDR2 and CDR3 domains of having the sequences set forth in SEQ ID NO:480, SEQ ID NO:481, and/or SEQ ID NO:482, respectively, or conservative amino acid substitutions thereof, and/or light chain CDR1, CDR2 and CDR3 domains having the sequences set forth in SEQ ID NO:483, SEQ ID NO:484, and/or SEQ ID NO:485, respectively, or conservative amino acid substitutions thereof.

In an embodiment, an anti-VISTA scFv domain comprises a V_{H} domain and a V_{L} domain, wherein the V_{H} domain is encoded by a sequence selected from the group consisting of SEQ ID NO:454, SEQ ID NO:462, SEQ ID NO:470, SEQ ID NO:478, and fragments, derivatives, variants, and conservative amino acid substitutions thereof, and the light chain variable region (V_{L}) comprises a sequence selected from the group consisting of SEQ ID NO:455, SEQ ID NO:463, SEQ ID NO:471, SEQ ID NO:479, and fragments, derivatives, variants, and conservative amino acid substitutions thereof. In an embodiment, an anti-VISTA scFv domain comprises a V_{H} region that is 99% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 99% identical to a sequence selected from the group consisting of SEQ ID NO:455, SEQ ID NO:463, SEQ ID NO:471, SEQ ID NO:479, and fragments, derivatives, and variants thereof. In an embodiment, an anti-VISTA scFv domain comprises a V_{H} region that is 98% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 98% identical to a sequence selected from the group consisting of SEQ ID NO:455, SEQ ID NO:463, SEQ ID NO:471, SEQ ID NO:479, and fragments, derivatives, and variants thereof. In an embodiment, an anti-VISTA scFv domain comprises a V_{H} region that is 97% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 97% identical to a sequence selected from the group consisting of SEQ ID NO:455, SEQ ID NO:463, SEQ ID NO:471, SEQ ID NO:479, and fragments, derivatives, and variants thereof. In an embodiment, an anti-VISTA scFv domain comprises a V_{H} region that is 96% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 96% identical to a sequence selected from the group consisting of SEQ ID NO:455, SEQ ID NO:463, SEQ ID NO:471, SEQ ID NO:479, and fragments, derivatives, and variants thereof. In an embodiment, an anti-VISTA scFv domain comprises a V_{H} region that is 95% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 95% identical to a sequence selected from the group consisting of SEQ ID NO:455, SEQ ID NO:463, SEQ ID NO:471, SEQ ID NO:479, and fragments, derivatives, and variants thereof. In an embodiment, an anti-VISTA scFv domain comprises a V_{H} region that is 90% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 90% identical to a sequence selected from the group consisting of SEQ ID NO:455, SEQ ID NO:463, SEQ ID NO:471, SEQ ID NO:479, and fragments, derivatives, and variants thereof. In an embodiment, an anti-VISTA scFv domain comprises a V_{H} region that is 85% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 85% identical to a sequence selected from the group consisting of SEQ ID NO:455, SEQ ID NO:463, SEQ ID NO:471, SEQ ID NO:479, and fragments, derivatives, and variants thereof. In an embodiment, an anti-VISTA scFv domain comprises a V_{H} region that is 80% identical to a sequence selected from the group consisting of SEQ ID NO:454, SEQ ID NO:462, SEQ ID NO:470, SEQ ID NO:478, and fragments, derivatives, and variants thereof, and a V_{L} region that is at least 80% identical to a sequence selected from the group consisting of SEQ ID NO:455, SEQ ID NO:463, SEQ ID NO:471, SEQ ID NO:479, and fragments, derivatives, and variants thereof.

In an embodiment, the scFv domain includes a scFv, V_{H}, V_{L}, or CDR domain of the antibodies 1B8, 2C12, 1A12, 3C5, 2B7, 2C12(H), 2C12(L), 1C9, 1D10, and variants, derivatives, and fragments thereof, the preparation and properties of each of which are described in U.S. Patent Application Publication No. US 2020/0407449 A1 and are incorporated by reference herein. Other sequences that may be employed for construction of alternative VISTA binding domains suitable for use with the present invention are described in U.S. Patent Application Publication No. US 2020/0407449 A1, the disclosures of which are incorporated by reference herein.

In an embodiment, the anti-VISTA binding domain includes an scFv, V_{H} and/or V_{L} sequence, or a heavy chain and/or a light chain CDR1, CDR2, and/or CDR3 sequence as disclosed in U.S. Patent Application Publication No. US 2017/0306024 A1, the disclosures of which are incorporated by reference herein.

### 11. Extracellular LRRC15 Binding Domains

In an embodiment, a CCR of the present invention comprises an extracellular domain, wherein the extracellular domain comprises a leucine-rich repeat-containing protein 15 (LRRC15) binding domain. LRRC15 is a cell surface protein with two known isoforms and is known to be expressed on stromal and cancer-associated fibroblasts in many solid tumors, including breast, head and neck, lung, and pancreatic tumors, as well as directly on a subset of cancer cells of mesenchymal origin, including sarcoma, melanoma, and glioblastoma, as described in Purcell, et al., Cancer Res. 2018, 78, 4059-72, the disclosure of which is incorporated by reference herein. In an embodiment, the CCR comprises an extracellular domain that binds to human LRRC15. In an embodiment, the extracellular domain binds to murine LRRC15. In an embodiment, the extracellular LRRC15 binding domain is a scFv domain. In an embodiment, a CCR of the present invention comprises a construct as shown in FIG. 34, wherein the V_{H} and V_{L} domains are anti-LRRC15 V_{H} and V_{L} domains, and the linker is as described herein.

In an embodiment, the anti-LRRC15 binding domain includes a V_{H} and/or V_{L} sequence or a heavy chain and/or a light chain CDR1, CDR2, and/or CDR3 sequence as disclosed in U.S. Pat. No. 10,188,660, the disclosures of which are incorporated by reference herein. In an embodiment, the anti-LRRC15 binding domain includes a V_{H} and/or V_{L} sequence or a heavy chain and/or a light chain CDR1, CDR2, and/or CDR3 sequence, or a nucleotide encoding such a sequence, for the antibodies huM25, huAD208.4.1, huAD208.12.1, huAD208.14.1, hu139.10, muAD210.40.9, or muAD209.9.1, each as disclosed in U.S. Pat. No. 10,188,660, the disclosures of which are incorporated by reference herein. The amino acid sequences of exemplary LRRC15 binding scFv domains are provided in Table 54.

TABLE 54
Amino acid sequences of exemplary LRRC15 binding scFv domains.

| Identifier | Sequence (One-Letter Amino Acid Symbols) | |
|---|---|---|
| SEQ ID NO: 486 | EVQLVQSGAE VKKPGASVKV SCKASGYKFS SYWIEWVKQA PGQGLEWIGE ILPGSDTTNY | 60 |
| Anti-LRRC15 | NEKFKDRATF TSDTSINTAY MELSRLRSDD TAVYYCARDR GNYRAWFGYW GQGTLVTVSS | 120 |
| huM25 variable | | |
| heavy chain | | |
| SEQ ID NO: 487 | DIQMTQSPSS LSASVGDRVT ITCRASQDIS NYLNWYQQKP GGAVKFLIYY TSRLHSGVPS | 60 |
| Anti-LRRC15 | RFSGSGSGTD YTLTISSLQP EDFATYFCQQ GEALPWTFGG GTKVEIK | 107 |
| huM25 variable | | |
| light chain | | |
| SEQ ID NO: 488 | SYWIE | 5 |
| Anti-LRRC15 | | |
| antibody huM25 | | |
| variable heavy | | |
| chain CDR1 | | |
| SEQ ID NO: 489 | EILPGSDTTN YNEKFKD | 17 |
| Anti-LRRC15 | | |
| antibody huM25 | | |
| variable heavy | | |
| chain CDR2 | | |
| SEQ ID NO: 490 | DRGNYRAWFG Y | 11 |
| Anti-LRRC15 | | |
| antibody huM25 | | |
| variable heavy | | |
| chain CDR3 | | |
| SEQ ID NO: 491 | RASQDISNYL N | 11 |
| Anti-LRRC15 | | |
| antibody huM25 | | |
| variable light | | |
| chain CDR1 | | |
| SEQ ID NO: 492 | YTSRLHS | 7 |
| Anti-LRRC15 | | |
| antibody huM25 | | |
| variable light | | |
| chain CDR2 | | |
| SEQ ID NO: 493 | QQGEALPWT | 9 |
| Anti-LRRC15 | | |
| antibody huM25 | | |
| variable light | | |
| chain CDR3 | | |
| SEQ ID NO: 494 | EVQLVQSGAE VKKPGSSVKV SCKASGFTFT DYYIEWVKQA PGQGLEWIGL VYPYIGGTNY | 60 |
| Anti-LRRC15 | NQKFKGKATL TVDTSTTTAY MEMSSLRSED TAVYYCARGD NKYDAMDYWG QGTTVTVSS | 119 |
| huAD208.4.1 | | |
| variable heavy | | |
| chain | | |
| SEQ ID NO: 495 | DIVLTQSPDS LAVSLGERAT INCRASQSVS TSSYSYMHWY QQKPGQPPKL LIKYASSLES | 60 |
| Anti-LRRC15 | GVPDRFSGSG SGTDFTLTIS SLQAEDVAVY YCEQSWEIRT FGGGTKVEIK | 110 |
| huAD208.4.1 | | |
| variable light | | |
| chain | | |
| SEQ ID NO: 496 | DYYIH | 5 |
| Anti-LRRC15 | | |
| antibody | | |
| huAD208.4.1 | | |
| variable heavy | | |
| chain CDR1 | | |
| SEQ ID NO: 497 | LVYPYIGGTN YNQKFKG | 17 |
| Anti-LRRC15 | | |
| antibody | | |
| huAD208.4.1 | | |
| variable heavy | | |
| chain CDR2 | | |
| SEQ ID NO: 498 | GDNKYDAMDY | 10 |
| Anti-LRRC15 | | |
| antibody | | |
| huAD208.4.1 | | |
| variable heavy | | |
| chain CDR3 | | |
| SEQ ID NO: 499 | RASQSVSTSS YSYMH | 15 |
| Anti-LRRC15 | | |
| antibody | | |
| huAD208.4.1 | | |
| variable light | | |
| chain CDR1 | | |
| SEQ ID NO: 500 | YASSLES | 7 |
| Anti-LRRC15 | | |
| antibody | | |
| huAD208.4.1 | | |
| variable light | | |
| chain CDR2 | | |
| SEQ ID NO: 501 | EQSWEIRT | 8 |
| Anti-LRRC15 | | |
| antibody | | |
| huAD208.4.1 | | |
| variable light | | |
| chain CDR3 | | |
| SEQ ID NO: 502 | EVQLVQSGAE VKKPGSSVKV SCKASGYTFT NYWMEWVKQA PGQGLEWIGM IHPNSGSTKH | 60 |
| Anti-LRRC15 | NEKFRGKATL TVDESTTTAY MELSSLRSED TAVYYCARSD FGNYRWYFDV WGQGTTVTVS | 120 |
| huAD208.12.1 | S | 121 |
| variable heavy | | |
| chain | | |
| SEQ ID NO: 503 | EIVLTQSPAT LSLSPGERAT LSCRASQSSS NNLHWYQQKP GQAPRVLIKY VSQSISGIPA | 60 |
| Anti-LRRC15 | RFSGSGSGTD FTLTISSLEP EDFAVYFCQQ SNSWPFTFGQ GTKLEIK | 107 |
| huAD208.12.1 | | |
| variable light | | |
| chain | | |
| SEQ ID NO: 504 | NYWMH | 5 |
| Anti-LRRC15 | | |
| antibody | | |
| huAD208.12.1 | | |
| variable heavy | | |
| chain CDR1 | | |
| SEQ ID NO: 505 | MIHPNSGSTK HNEKFRG | 17 |
| Anti-LRRC15 | | |
| antibody | | |
| huAD208.12.1 | | |
| variable heavy | | |
| chain CDR2 | | |
| SEQ ID NO: 506 | SDFGNYRWYF DV | 12 |
| Anti-LRRC15 | | |
| antibody | | |
| huAD208.12.1 | | |
| variable heavy | | |
| chain CDR3 | | |
| SEQ ID NO: 507 | RASQSSSNNL H | 11 |
| Anti-LRRC15 | | |
| antibody | | |
| huAD208.12.1 | | |
| variable light | | |
| chain CDR1 | | |
| SEQ ID NO: 508 | YVSQSIS | 7 |
| Anti-LRRC15 | | |
| antibody | | |
| huAD208.12.1 | | |
| variable light | | |
| chain CDR2 | | |
| SEQ ID NO: 509 | QQSNSWPFT | 9 |
| Anti-LRRC15 | | |
| antibody | | |
| huAD208.12.1 | | |
| variable light | | |
| chain CDR3 | | |
| SEQ ID NO: 510 | EVQLVQSGAE VKKPGSSVKV SCKASGFTFT DYYIEWVKQA PGQGLEWIGL VYPYIGGSSY | 60 |
| Anti-LRRC15 | NQQFKGKATL TVDTSTSTAY MELSSLRSED TAVYYCARGD NNYDAMDYWG QGTTVTVSS | 119 |
| huAD208.14.1 | | |
| variable heavy | | |
| chain | | |
| SEQ ID NO: 511 | DIVLTQSPDS LAVSLGERAT ISCRASQSVS TSTYNYMHWY QQKPGQPPKL LVKYASNLES | 60 |
| Anti-LRRC15 | GVPDRFSGSG SGTDFTLTIS SLQAEDVAVY YCHHTWEIRT FGGGTKVEIK | 110 |
| huAD208.14.1 | | |
| variable light | | |
| chain | | |
| SEQ ID NO: 512 | DYYIH | 5 |
| Anti-LRRC15 | | |
| antibody | | |
| huAD208.14.1 | | |
| variable heavy | | |
| chain CDR1 | | |
| SEQ ID NO: 513 | LVYPYIGGSS YNQQFKG | 17 |
| Anti-LRRC15 | | |
| antibody | | |
| huAD208.14.1 | | |
| variable heavy | | |
| chain CDR2 | | |
| SEQ ID NO: 514 | GDNNYDAMDY | 10 |
| Anti-LRRC15 | | |
| antibody | | |
| huAD208.14.1 | | |
| variable heavy | | |
| chain CDR3 | | |
| SEQ ID NO: 515 | RASQSVSTST YNYMH | 15 |
| Anti-LRRC15 | | |
| antibody | | |
| huAD208.14.1 | | |
| variable light | | |
| chain CDR1 | | |
| SEQ ID NO: 516 | YASNLES | 7 |
| Anti-LRRC15 | | |
| antibody | | |
| huAD208.14.1 | | |
| variable light | | |
| chain CDR2 | | |
| SEQ ID NO: 517 | HHTWEIRT | 8 |
| Anti-LRRC15 | | |
| antibody | | |
| huAD208.14.1 | | |
| variable light | | |
| chain CDR3 | | |
| SEQ ID NO: 518 | EVQLVESGGG LVQPGGSLRL SCAVSGFSLT SYGVEWVRQA TGKGLEWLGV IWAGGSTNYN | 60 |
| Anti-LRRC15 | SALMSRLTIS KENAKSSVYL QMNSLRAGDT AMYYCATHMI TEDYYGMDYW GQGTTVTVSS | 120 |
| hu139.10 | | |
| variable heavy | | |
| chain | | |
| SEQ ID NO: 519 | DIVMTQSPDS LAVSLGERAT INCKSSQSLL NSRTRKNYLA WYQQKPGQSP KLLIYWASTR | 60 |
| Anti-LRRC15 | ESGVPDRFSG SGSGTDFTLT ISSLQAEDVA VYYCKQSYNL PTFGGGTKVE IK | 112 |
| hu139.10 | | |
| variable light | | |
| chain | | |
| SEQ ID NO: 520 | SYGVH | 5 |
| Anti-LRRC15 | | |
| antibody | | |
| hu139.10 | | |
| variable heavy | | |
| chain CDR1 | | |
| SEQ ID NO: 521 | VIWAGGSTNY NSALMS | 16 |
| Anti-LRRC15 | | |
| antibody | | |
| hu139.10 | | |
| variable heavy | | |
| chain CDR2 | | |
| SEQ ID NO: 522 | HMITEDYYGM DY | 12 |
| Anti-LRRC15 | | |
| antibody | | |
| hu139.10 | | |
| variable heavy | | |
| chain CDR3 | | |
| SEQ ID NO: 523 | KSSQSLLNSR TRKNYLA | 17 |
| Anti-LRRC15 | | |
| antibody | | |
| hu139.10 | | |
| variable light | | |
| chain CDR1 | | |
| SEQ ID NO: 524 | WASTRES | 7 |
| Anti-LRRC15 | | |
| antibody | | |
| hu139.10 | | |
| variable light | | |
| chain CDR2 | | |
| SEQ ID NO: 525 | KQSYNLPT | 8 |
| Anti-LRRC15 | | |
| antibody | | |
| hu139.10 | | |
| variable light | | |
| chain CDR3 | | |
| SEQ ID NO: 526 | QVQLQQSGAE LVRPGTSVKI SCKASGYDFT NYWLGWVKQR PGHGLEWIGD IYPGGGNTYY | 60 |
| Anti-LRRC15 | NEKLKGKATL TADKSSSTAY IHLISLTSED SSVYFCARWG DKKGNYFAYW GQGTLVTVSA | 120 |
| muAD210.40.9 | | |
| variable heavy | | |
| chain | | |
| SEQ ID NO: 527 | QIVLTQSPAI MSASLGERVT MTCTASSSVY SSYLEWYQQK PGSSPKLWIY STSNLASGVP | 60 |
| Anti-LRRC15 | GRFSGSGSGT SYSLTISSME AEDAATYYCH QYHRSPTFGG GTKLEIK | 107 |
| muAD210.40.9 | | |
| variable light | | |
| chain | | |
| SEQ ID NO: 528 | NYWLG | 5 |
| Anti-LRRC15 | | |
| antibody | | |
| muAD210.40.9 | | |
| variable heavy | | |
| chain CDR1 | | |
| SEQ ID NO: 529 | DIYPGGGNTY YNEKLKG | 17 |
| Anti-LRRC15 | | |
| antibody | | |
| muAD210.40.9 | | |
| variable heavy | | |
| chain CDR2 | | |
| SEQ ID NO: 530 | WGDKKGNYFA Y | 11 |
| Anti-LRRC15 | | |
| antibody | | |
| muAD210.40.9 | | |
| variable heavy | | |
| chain CDR3 | | |
| SEQ ID NO: 531 | TASSSVYSSY LH | 12 |
| Anti-LRRC15 | | |
| antibody | | |
| muAD210.40.9 | | |
| variable light | | |
| chain CDR1 | | |
| SEQ ID NO: 532 | STSNLAS | 7 |
| Anti-LRRC15 | | |
| antibody | | |
| muAD210.40.9 | | |
| variable light | | |
| chain CDR2 | | |
| SEQ ID NO: 533 | HQYHRSPT | 8 |
| Anti-LRRC15 | | |
| antibody | | |
| muAD210.40.9 | | |
| variable light | | |
| chain CDR3 | | |
| SEQ ID NO: 534 | QIQLVQSGPE LKKPGETVKI SCKASGFAIT NFGMNWVKQA PGKGLKWMGW INLYTGEPTF | 60 |
| Anti-LRRC15 | ADDFKGRFAF SLETSASTAY LQINNLKNED TVIYFCARKG ETYYRYDGFA YWGQGTLVTV | 120 |
| muAD209.9.1 | SA | 122 |
| variable heavy | | |
| chain | | |
| SEQ ID NO: 535 | DIVMTQAAPS VPVTPGESVS ISCRSSKSLL HSNGNTELYW FLQRPGQSPQ LLIYRMSNLA | 60 |
| Anti-LRRC15 | SGVPDRFSGS GSGTAFTLRI SRVEAEDVGV YYCMQLLEYP YTFGGGTKLE IE | 112 |
| muAD209.9.1 | | |
| variable light | | |
| chain | | |
| SEQ ID NO: 536 | NFGMN | 5 |
| Anti-LRRC15 | | |
| antibody | | |
| muAD209.9.1 | | |
| variable heavy | | |
| chain CDR1 | | |
| SEQ ID NO: 537 | WINLYTGEPT FADDFKG | 17 |
| Anti-LRRC15 | | |
| antibody | | |
| muAD209.9.1 | | |
| variable heavy | | |
| chain CDR2 | | |
| SEQ ID NO: 538 | KGETYYRYDG FAY | 13 |
| Anti-LRRC15 | | |
| antibody | | |
| muAD209.9.1 | | |
| variable heavy | | |
| chain CDR3 | | |
| SEQ ID NO: 539 | RSSKSLLHSN GNTHLY | 16 |
| Anti-LRRC15 | | |
| antibody | | |
| muAD209.9.1 | | |
| variable light | | |
| chain CDR1 | | |
| SEQ ID NO: 540 | RMSNLAS | 7 |
| Anti-LRRC15 | | |
| antibody | | |
| muAD209.9.1 | | |
| variable light | | |
| chain CDR2 | | |
| SEQ ID NO: 541 | MQLLEYPYT | 9 |
| Anti-LRRC15 | | |
| antibody | | |
| muAD209.9.1 | | |
| variable light | | |
| chain CDR3 | | |

In an embodiment, an anti-LRRC15 scFv domain comprises a heavy chain variable region (V_{H}) domain and/or a light chain variable region (V_{L}) domain of the antibody huM25, or variants, derivatives, fragments, or conservative amino acid substitutions thereof. In an embodiment, an anti-LRRC15 scFv domain comprises a V_{H} domain and a V_{L} domain, wherein the V_{H} domain comprises the sequence shown in SEQ ID NO:486, or conservative amino acid substitutions thereof, and the light chain variable region (V_{L}) comprises the sequence shown in SEQ ID NO:487, or conservative amino acid substitutions thereof. In an embodiment, an anti-LRRC15 scFv domain comprises V_{H} and V_{L} regions that are each at least 99% identical to the sequences shown in SEQ ID NO:486 and SEQ ID NO:487, respectively. In an embodiment, an anti-LRRC15 scFv domain comprises V_{H} and V_{L} regions that are each at least 98% identical to the sequences shown in SEQ ID NO:486 and SEQ ID NO:487, respectively. In an embodiment, an anti-LRRC15 scFv domain comprises V_{H} and V_{L} regions that are each at least 97% identical to the sequences shown in SEQ ID NO:486 and SEQ ID NO:487, respectively. In an embodiment, an anti-LRRC15 scFv domain comprises V_{H} and V_{L} regions that are each at least 96% identical to the sequences shown in SEQ ID NO:486 and SEQ ID NO:487, respectively. In an embodiment, an anti-LRRC15 scFv domain comprises V_{H} and V_{L} regions that are each at least 95% identical to the sequences shown in SEQ ID NO:486 and SEQ ID NO:487, respectively. In an embodiment, an anti-LRRC15 scFv domain comprises V_{H} and V_{L} regions that are each at least 90% identical to the sequences shown in SEQ ID NO:486 and SEQ ID NO:487, respectively. In an embodiment, an anti-LRRC15 scFv domain comprises V_{H} and V_{L} regions that are each at least 85% identical to the sequences shown in SEQ ID NO:486 and SEQ ID NO:487, respectively. In an embodiment, an anti-LRRC15 scFv domain comprises V_{H} and V_{L} regions that are each at least 80% identical to the sequences shown in SEQ ID NO:486 and SEQ ID NO:487, respectively.

In an embodiment, an anti-LRRC15 scFv domain comprises the heavy chain CDR1, CDR2 and CDR3 domains and the light chain CDR1, CDR2, and CDR3 domains, or conservative amino acid substitutions thereof, of the antibody huM25. In an embodiment, an anti-LRRC15 scFv domain comprises the heavy chain CDR1, CDR2 and CDR3 domains of having the sequences set forth in SEQ ID NO:488, SEQ ID NO:489, and/or SEQ ID NO:490, respectively, or conservative amino acid substitutions thereof, and/or light chain CDR1, CDR2 and CDR3 domains having the sequences set forth in SEQ ID NO:491, SEQ ID NO:492, and/or SEQ ID NO:493, respectively, or conservative amino acid substitutions thereof.

In an embodiment, an anti-LRRC15 scFv domain comprises a heavy chain variable region (V_{H}) domain and/or a light chain variable region (V_{L}) domain of the antibody huAD208.4.1, or variants, derivatives, fragments, or conservative amino acid substitutions thereof. In an embodiment, an anti-LRRC15 scFv domain comprises a V_{H} domain and a V_{L} domain, wherein the V_{H} domain comprises the sequence shown in SEQ ID NO:494, or conservative amino acid substitutions thereof, and the light chain variable region (V_{L}) comprises the sequence shown in SEQ ID NO:495, or conservative amino acid substitutions thereof. In an embodiment, an anti-LRRC15 scFv domain comprises V_{H} and V_{L} regions that are each at least 99% identical to the sequences shown in SEQ ID NO:494 and SEQ ID NO:495, respectively. In an embodiment, an anti-LRRC15 scFv domain comprises V_{H} and V_{L} regions that are each at least 98% identical to the sequences shown in SEQ ID NO:494 and SEQ ID NO:495, respectively. In an embodiment, an anti-LRRC15 scFv domain comprises V_{H} and V_{L} regions that are each at least 97% identical to the sequences shown in SEQ ID NO:494 and SEQ ID NO:495, respectively. In an embodiment, an anti-LRRC15 scFv domain comprises V_{H} and V_{L} regions that are each at least 96% identical to the sequences shown in SEQ ID NO:494 and SEQ ID NO:495, respectively. In an embodiment, an anti-LRRC15 scFv domain comprises V_{H} and V_{L} regions that are each at least 95% identical to the sequences shown in SEQ ID NO:494 and SEQ ID NO:495, respectively. In an embodiment, an anti-LRRC15 scFv domain comprises V_{H} and V_{L} regions that are each at least 90% identical to the sequences shown in SEQ ID NO:494 and SEQ ID NO:495, respectively. In an embodiment, an anti-LRRC15 scFv domain comprises V_{H} and V_{L} regions that are each at least 85% identical to the sequences shown in SEQ ID NO:494 and SEQ ID NO:495, respectively. In an embodiment, an anti-LRRC15 scFv domain comprises V_{H} and V_{L} regions that are each at least 80% identical to the sequences shown in SEQ ID NO:494 and SEQ ID NO:495, respectively.

In an embodiment, an anti-LRRC15 scFv domain comprises the heavy chain CDR1, CDR2 and CDR3 domains and the light chain CDR1, CDR2, and CDR3 domains, or conservative amino acid substitutions thereof, of the antibody huAD208.4.1. In an embodiment, an anti-LRRC15 scFv domain comprises the heavy chain CDR1, CDR2 and CDR3 domains of having the sequences set forth in SEQ ID NO:496, SEQ ID NO:497, and/or SEQ ID NO:498, respectively, or conservative amino acid substitutions thereof, and/or light chain CDR1, CDR2 and CDR3 domains having the sequences set forth in SEQ ID NO:499, SEQ ID NO:500, and/or SEQ ID NO:501, respectively, or conservative amino acid substitutions thereof.

In an embodiment, an anti-LRRC15 scFv domain comprises a heavy chain variable region (V_{H}) domain and/or a light chain variable region (V_{L}) domain of the antibody huAD208.12.1, or variants, derivatives, fragments, or conservative amino acid substitutions thereof. In an embodiment, an anti-LRRC15 scFv domain comprises a V_{H} domain and a V_{L} domain, wherein the V_{H} domain comprises the sequence shown in SEQ ID NO:502, or conservative amino acid substitutions thereof, and the light chain variable region (V_{L}) comprises the sequence shown in SEQ ID NO:503, or conservative amino acid substitutions thereof. In an embodiment, an anti-LRRC15 scFv domain comprises V_{H} and V_{L} regions that are each at least 99% identical to the sequences shown in SEQ ID NO:502 and SEQ ID NO:503, respectively. In an embodiment, an anti-LRRC15 scFv domain comprises V_{H} and V_{L} regions that are each at least 98% identical to the sequences shown in SEQ ID NO:502 and SEQ ID NO:503, respectively. In an embodiment, an anti-LRRC15 scFv domain comprises V_{H} and V_{L} regions that are each at least 97% identical to the sequences shown in SEQ ID NO:502 and SEQ ID NO:503, respectively. In an embodiment, an anti-LRRC15 scFv domain comprises V_{H} and V_{L} regions that are each at least 96% identical to the sequences shown in SEQ ID NO:502 and SEQ ID NO:503, respectively. In an embodiment, an anti-LRRC15 scFv domain comprises V_{H} and V_{L} regions that are each at least 95% identical to the sequences shown in SEQ ID NO:502 and SEQ ID NO:503, respectively. In an embodiment, an anti-LRRC15 scFv domain comprises V_{H} and V_{L} regions that are each at least 90% identical to the sequences shown in SEQ ID NO:502 and SEQ ID NO:503, respectively. In an embodiment, an anti-LRRC15 scFv domain comprises V_{H} and V_{L} regions that are each at least 85% identical to the sequences shown in SEQ ID NO:502 and SEQ ID NO:503, respectively. In an embodiment, an anti-LRRC15 scFv domain comprises V_{H} and V_{L} regions that are each at least 80% identical to the sequences shown in SEQ ID NO:502 and SEQ ID NO:503, respectively.

In an embodiment, an anti-LRRC15 scFv domain comprises the heavy chain CDR1, CDR2 and CDR3 domains and the light chain CDR1, CDR2, and CDR3 domains, or conservative amino acid substitutions thereof, of the antibody huAD208.12.1. In an embodiment, an anti-LRRC15 scFv domain comprises the heavy chain CDR1, CDR2 and CDR3 domains of having the sequences set forth in SEQ ID NO:504, SEQ ID NO:505, and/or SEQ ID NO:506, respectively, or conservative amino acid substitutions thereof, and/or light chain CDR1, CDR2 and CDR3 domains having the sequences set forth in SEQ ID NO:507, SEQ ID NO:508, and/or SEQ ID NO:509, respectively, or conservative amino acid substitutions thereof.

In an embodiment, an anti-LRRC15 scFv domain comprises a heavy chain variable region (V_{H}) domain and/or a light chain variable region (V_{L}) domain of the antibody huAD208.14.1, or variants, derivatives, fragments, or conservative amino acid substitutions thereof. In an embodiment, an anti-LRRC15 scFv domain comprises a V_{H} domain and a V_{L} domain, wherein the V_{H} domain comprises the sequence shown in SEQ ID NO:510, or conservative amino acid substitutions thereof, and the light chain variable region (V_{L}) comprises the sequence shown in SEQ ID NO:511, or conservative amino acid substitutions thereof. In an embodiment, an anti-LRRC15 scFv domain comprises V_{H} and V_{L} regions that are each at least 99% identical to the sequences shown in SEQ ID NO:510 and SEQ ID NO:511, respectively. In an embodiment, an anti-LRRC15 scFv domain comprises V_{H} and V_{L} regions that are each at least 98% identical to the sequences shown in SEQ ID NO:510 and SEQ ID NO:511, respectively. In an embodiment, an anti-LRRC15 scFv domain comprises V_{H} and V_{L} regions that are each at least 97% identical to the sequences shown in SEQ ID NO:510 and SEQ ID NO:511, respectively. In an embodiment, an anti-LRRC15 scFv domain comprises V_{H} and V_{L} regions that are each at least 96% identical to the sequences shown in SEQ ID NO:510 and SEQ ID NO:511, respectively. In an embodiment, an anti-LRRC15 scFv domain comprises V_{H} and V_{L} regions that are each at least 95% identical to the sequences shown in SEQ ID NO:510 and SEQ ID NO:511, respectively. In an embodiment, an anti-LRRC15 scFv domain comprises V_{H} and V_{L} regions that are each at least 90% identical to the sequences shown in SEQ ID NO:510 and SEQ ID NO:511, respectively. In an embodiment, an anti-LRRC15 scFv domain comprises V_{H} and V_{L} regions that are each at least 85% identical to the sequences shown in SEQ ID NO:510 and SEQ ID NO:511, respectively. In an embodiment, an anti-LRRC15 scFv domain comprises V_{H} and V_{L} regions that are each at least 80% identical to the sequences shown in SEQ ID NO:510 and SEQ ID NO:511, respectively.

In an embodiment, an anti-LRRC15 scFv domain comprises the heavy chain CDR1, CDR2 and CDR3 domains and the light chain CDR1, CDR2, and CDR3 domains, or conservative amino acid substitutions thereof, of the antibody huAD208.14.1. In an embodiment, an anti-LRRC15 scFv domain comprises the heavy chain CDR1, CDR2 and CDR3 domains of having the sequences set forth in SEQ ID NO:512, SEQ ID NO:513, and/or SEQ ID NO:514, respectively, or conservative amino acid substitutions thereof, and/or light chain CDR1, CDR2 and CDR3 domains having the sequences set forth in SEQ ID NO:515, SEQ ID NO:516, and/or SEQ ID NO:517, respectively, or conservative amino acid substitutions thereof.

In an embodiment, an anti-LRRC15 scFv domain comprises a heavy chain variable region (V_{H}) domain and/or a light chain variable region (V_{L}) domain of the antibody hu139.10, or variants, derivatives, fragments, or conservative amino acid substitutions thereof. In an embodiment, an anti-LRRC15 scFv domain comprises a V_{H} domain and a V_{L} domain, wherein the V_{H} domain comprises the sequence shown in SEQ ID NO:518, or conservative amino acid substitutions thereof, and the light chain variable region (V_{L}) comprises the sequence shown in SEQ ID NO:519, or conservative amino acid substitutions thereof. In an embodiment, an anti-LRRC15 scFv domain comprises V_{H} and V_{L} regions that are each at least 99% identical to the sequences shown in SEQ ID NO:518 and SEQ ID NO:519, respectively. In an embodiment, an anti-LRRC15 scFv domain comprises V_{H} and V_{L} regions that are each at least 98% identical to the sequences shown in SEQ ID NO:518 and SEQ ID NO:519, respectively. In an embodiment, an anti-LRRC15 scFv domain comprises V_{H} and V_{L} regions that are each at least 97% identical to the sequences shown in SEQ ID NO:518 and SEQ ID NO:519, respectively. In an embodiment, an anti-LRRC15 scFv domain comprises V_{H} and V_{L} regions that are each at least 96% identical to the sequences shown in SEQ ID NO:518 and SEQ ID NO:519, respectively. In an embodiment, an anti-LRRC15 scFv domain comprises V_{H} and V_{L} regions that are each at least 95% identical to the sequences shown in SEQ ID NO:518 and SEQ ID NO:519, respectively. In an embodiment, an anti-LRRC15 scFv domain comprises V_{H} and V_{L} regions that are each at least 90% identical to the sequences shown in SEQ ID NO:518 and SEQ ID NO:519, respectively. In an embodiment, an anti-LRRC15 scFv domain comprises V_{H} and V_{L} regions that are each at least 85% identical to the sequences shown in SEQ ID NO:518 and SEQ ID NO:519, respectively. In an embodiment, an anti-LRRC15 scFv domain comprises V_{H} and V_{L} regions that are each at least 80% identical to the sequences shown in SEQ ID NO:518 and SEQ ID NO:519, respectively.

In an embodiment, an anti-LRRC15 scFv domain comprises the heavy chain CDR1, CDR2 and CDR3 domains and the light chain CDR1, CDR2, and CDR3 domains, or conservative amino acid substitutions thereof, of the antibody hu139.10. In an embodiment, an anti-LRRC15 scFv domain comprises the heavy chain CDR1, CDR2 and CDR3 domains of having the sequences set forth in SEQ ID NO:520, SEQ ID NO:521, and/or SEQ ID NO:522, respectively, or conservative amino acid substitutions thereof, and/or light chain CDR1, CDR2 and CDR3 domains having the sequences set forth in SEQ ID NO:523, SEQ ID NO:524, and/or SEQ ID NO:525, respectively, or conservative amino acid substitutions thereof.

In an embodiment, an anti-LRRC15 scFv domain comprises a heavy chain variable region (V_{H}) domain and/or a light chain variable region (V_{L}) domain of the antibody muAD210.40.9, or variants, derivatives, fragments, or conservative amino acid substitutions thereof. In an embodiment, an anti-LRRC15 scFv domain comprises a V_{H} domain and a V_{L} domain, wherein the V_{H} domain comprises the sequence shown in SEQ ID NO:526, or conservative amino acid substitutions thereof, and the light chain variable region (V_{L}) comprises the sequence shown in SEQ ID NO:527, or conservative amino acid substitutions thereof. In an embodiment, an anti-LRRC15 scFv domain comprises V_{H} and V_{L} regions that are each at least 99% identical to the sequences shown in SEQ ID NO:526 and SEQ ID NO:527, respectively. In an embodiment, an anti-LRRC15 scFv domain comprises V_{H} and V_{L} regions that are each at least 98% identical to the sequences shown in SEQ ID NO:526 and SEQ ID NO:527, respectively. In an embodiment, an anti-LRRC15 scFv domain comprises V_{H} and V_{L} regions that are each at least 97% identical to the sequences shown in SEQ ID NO:526 and SEQ ID NO:527, respectively. In an embodiment, an anti-LRRC15 scFv domain comprises V_{H} and V_{L} regions that are each at least 96% identical to the sequences shown in SEQ ID NO:526 and SEQ ID NO:527, respectively. In an embodiment, an anti-LRRC15 scFv domain comprises V_{H} and V_{L} regions that are each at least 95% identical to the sequences shown in SEQ ID NO:526 and SEQ ID NO:527, respectively. In an embodiment, an anti-LRRC15 scFv domain comprises V_{H} and V_{L} regions that are each at least 90% identical to the sequences shown in SEQ ID NO:526 and SEQ ID NO:527, respectively. In an embodiment, an anti-LRRC15 scFv domain comprises V_{H} and V_{L} regions that are each at least 85% identical to the sequences shown in SEQ ID NO:526 and SEQ ID NO:527, respectively. In an embodiment, an anti-LRRC15 scFv domain comprises V_{H} and V_{L} regions that are each at least 80% identical to the sequences shown in SEQ ID NO:526 and SEQ ID NO:527, respectively.

In an embodiment, an anti-LRRC15 scFv domain comprises the heavy chain CDR1, CDR2 and CDR3 domains and the light chain CDR1, CDR2, and CDR3 domains, or conservative amino acid substitutions thereof, of the antibody muAD210.40.9. In an embodiment, an anti-LRRC15 scFv domain comprises the heavy chain CDR1, CDR2 and CDR3 domains of having the sequences set forth in SEQ ID NO:528, SEQ ID NO:529, and/or SEQ ID NO:530, respectively, or conservative amino acid substitutions thereof, and/or light chain CDR1, CDR2 and CDR3 domains having the sequences set forth in SEQ ID NO:531, SEQ ID NO:532, and/or SEQ ID NO:533, respectively, or conservative amino acid substitutions thereof.

In an embodiment, an anti-LRRC15 scFv domain comprises a heavy chain variable region (V_{H}) domain and/or a light chain variable region (V_{L}) domain of the antibody muAD209.9.1, or variants, derivatives, fragments, or conservative amino acid substitutions thereof. In an embodiment, an anti-LRRC15 scFv domain comprises a V_{H} domain and a V_{L} domain, wherein the V_{H} domain comprises the sequence shown in SEQ ID NO:534, or conservative amino acid substitutions thereof, and the light chain variable region (V_{L}) comprises the sequence shown in SEQ ID NO:535, or conservative amino acid substitutions thereof. In an embodiment, an anti-LRRC15 scFv domain comprises V_{H} and V_{L} regions that are each at least 99% identical to the sequences shown in SEQ ID NO:534 and SEQ ID NO:535, respectively. In an embodiment, an anti-LRRC15 scFv domain comprises V_{H} and V_{L} regions that are each at least 98% identical to the sequences shown in SEQ ID NO:534 and SEQ ID NO:535, respectively. In an embodiment, an anti-LRRC15 scFv domain comprises V_{H} and V_{L} regions that are each at least 97% identical to the sequences shown in SEQ ID NO:534 and SEQ ID NO:535, respectively. In an embodiment, an anti-LRRC15 scFv domain comprises V_{H} and V_{L} regions that are each at least 96% identical to the sequences shown in SEQ ID NO:534 and SEQ ID NO:535, respectively. In an embodiment, an anti-LRRC15 scFv domain comprises V_{H} and V_{L} regions that are each at least 95% identical to the sequences shown in SEQ ID NO:534 and SEQ ID NO:535, respectively. In an embodiment, an anti-LRRC15 scFv domain comprises V_{H} and V_{L} regions that are each at least 90% identical to the sequences shown in SEQ ID NO:534 and SEQ ID NO:535, respectively. In an embodiment, an anti-LRRC15 scFv domain comprises V_{H} and V_{L} regions that are each at least 85% identical to the sequences shown in SEQ ID NO:534 and SEQ ID NO:535, respectively. In an embodiment, an anti-LRRC15 scFv domain comprises V_{H} and V_{L} regions that are each at least 80% identical to the sequences shown in SEQ ID NO:534 and SEQ ID NO:535, respectively.

In an embodiment, an anti-LRRC15 scFv domain comprises the heavy chain CDR1, CDR2 and CDR3 domains and the light chain CDR1, CDR2, and CDR3 domains, or conservative amino acid substitutions thereof, of the antibody muAD209.9.1. In an embodiment, an anti-LRRC15 scFv domain comprises the heavy chain CDR1, CDR2 and CDR3 domains of having the sequences set forth in SEQ ID NO:536, SEQ ID NO:537, and/or SEQ ID NO:538, respectively, or conservative amino acid substitutions thereof, and/or light chain CDR1, CDR2 and CDR3 domains having the sequences set forth in SEQ ID NO:539, SEQ ID NO:540, and/or SEQ ID NO:541, respectively, or conservative amino acid substitutions thereof.

In an embodiment, an anti-LRRC15 scFv domain comprises a V_{H} domain and a V_{L} domain, wherein the V_{H} domain is encoded by a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, variants, and conservative amino acid substitutions thereof, and the light chain variable region (V_{L}) comprises a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, variants, and conservative amino acid substitutions thereof. In an embodiment, an anti-LRRC15 scFv domain comprises a V_{H} region that is 99% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 99% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof. In an embodiment, an anti-LRRC15 scFv domain comprises a V_{H} region that is 98% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 98% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof. In an embodiment, an anti-LRRC15 scFv domain comprises a V_{H} region that is 97% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 97% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof. In an embodiment, an anti-LRRC15 scFv domain comprises a V_{H} region that is 96% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 96% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof. In an embodiment, an anti-LRRC15 scFv domain comprises a V_{H} region that is 95% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 95% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof. In an embodiment, an anti-LRRC15 scFv domain comprises a V_{H} region that is 90% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 90% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof. In an embodiment, an anti-LRRC15 scFv domain comprises a V_{H} region that is 85% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 85% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof. In an embodiment, an anti-LRRC15 scFv domain comprises a V_{H} region that is 80% identical to a sequence selected from the group consisting of SEQ ID NO:486, SEQ ID NO:494, SEQ ID NO:502, SEQ ID NO:510, SEQ ID NO:518, SEQ ID NO:526, SEQ ID NO:534, and fragments, derivatives, and variants thereof, and a V_{L} region that is at least 80% identical to a sequence selected from the group consisting of SEQ ID NO:487, SEQ ID NO:495, SEQ ID NO:503, SEQ ID NO:511, SEQ ID NO:519, SEQ ID NO:527, SEQ ID NO:535, and fragments, derivatives, and variants thereof.

### 12. Extracellular B7-H3 Binding Domains

In an embodiment, a CCR of the present invention comprises an extracellular domain, wherein the extracellular domain comprises a B7-H3 (also known as CD276) binding domain. B7-H3 (137 homology 3) is a cell surface glycoprotein expressed on antigen-presenting cells and is known to play a role in both immune evasion and cancer progression, as described in Castellanos, et al., Am. J. Clin. Exp. Immunol. 2017, 6, 66-75, the disclosure of which is incorporated by reference herein. Exon duplication in humans results in the expression of two B7-H3 isoforms having either a single IgV-IgC-like domain (2IgB7-H3 isoform) or a IgV-IgC-IgV-IgC-like domain (4IgB7-H3 isoform) containing several conserved cysteine residues. The predominant B7-H3 isoform in human tissues and cell lines is the 4IgB7-H3 isoform, as described in Steinberger, et al., J. Immunol. 2004, 172, 2352-59, the disclosure of which is incorporated by reference herein. In an embodiment, the CCR comprises an extracellular domain that binds to human B7-H3. In an embodiment, the CCR comprises an extracellular domain that binds to human 4IgB7-H3. In an embodiment, the CCR comprises an extracellular domain that binds to human 2IgB7-H3. In an embodiment, the extracellular domain binds to murine B7-H3. In an embodiment, the extracellular B7-H3 binding domain is a scFv domain. In an embodiment, a CCR of the present invention comprises a construct as shown in FIG. 34, wherein the V_{H} and V_{L} domains are anti-B7-H3 V_{H} and V_{L} domains, and the linker is as described herein.

In an embodiment, the anti-B7-H3 binding domain includes a V_{H} and/or V_{L} sequence or a heavy chain and/or a light chain CDR1, CDR2, and/or CDR3 sequence as disclosed in U.S. Pat. No. 10,730,945, the disclosures of which are incorporated by reference herein. In an embodiment, the anti-B7-H3 binding domain includes a V_{H} and/or V_{L} sequence or a heavy chain and/or a light chain CDR1, CDR2, and/or CDR3 sequence, or a nucleotide encoding such a sequence, for the antibodies BRCA84D (including BRCA84D-1 and BRCA84D-2 and their variants), BRCA68D, BRCA69D, PRCA157, TES7, OVCA22, GB8, or SG27, humanized or murine, including their variants, each as disclosed in U.S. Pat. No. 10,730,945, the disclosures of which are incorporated by reference herein. The amino acid sequences of exemplary B7-H3 binding scFv domains are provided in Table 55.

TABLE 55
Amino acid sequences of exemplary B7-H3 binding scFv domains.

| Identifier | Sequence (One-Letter Amino Acid Symbols) | |
|---|---|---|
| SEQ ID NO: 542 | EVQLVESGGG LVQPGGSLRL SCAASGFTFS SFGMEWVRQA PGKGLEWVAY ISSDSSAIYY | 60 |
| Anti-B7-H3 | ADTVKGRFTI SRDNAKNSLY LQMNSLRDED TAVYYCARGR ENIYYGSRLD YWGQGTTVTV | 120 |
| antibody | SS | 122 |
| hBRCA84D | | |
| variable heavy | | |
| chain | | |
| SEQ ID NO: 543 | DIQLTQSPSF LSASVGDRVT ITCKASQNVD TNVAWYQQKP GQAPKALIYS ASYRYSGVPS | 60 |
| Anti-B7-H3 | RFSGSGSGTD FTLTISSLQP EDFATYYCQQ YNNYPFTFGQ GTKLEIK | 107 |
| antibody | | |
| hBRCA84D | | |
| variable light | | |
| chain | | |
| SEQ ID NO: 544 | FGMH | 4 |
| Anti-B7-H3 | | |
| antibody | | |
| hBRCA84D | | |
| variable heavy | | |
| chain CDR1 | | |
| SEQ ID NO: 545 | YISSDSSAIY YADTVK | 16 |
| Anti-B7-H3 | | |
| antibody | | |
| hBRCA84D | | |
| variable heavy | | |
| chain CDR2 | | |
| SEQ ID NO: 546 | GRENIYYGSR LDY | 13 |
| Anti-B7-H3 | | |
| antibody | | |
| hBRCA84D | | |
| variable heavy | | |
| chain CDR3 | | |
| SEQ ID NO: 547 | KASQNVDTNV A | 11 |
| Anti-B7-H3 | | |
| antibody | | |
| hBRCA84D | | |
| variable light | | |
| chain CDR1 | | |
| SEQ ID NO: 548 | SASYRYS | 7 |
| Anti-B7-H3 | | |
| antibody | | |
| hBRCA84D | | |
| variable light | | |
| chain CDR2 | | |
| SEQ ID NO: 549 | QQYNNYPFT | 9 |
| Anti-B7-H3 | | |
| antibody | | |
| hBRCA84D | | |
| variable light | | |
| chain CDR3 | | |
| SEQ ID NO: 550 | DVQLVESGGG LVQPGGSRKL SCAASGFTFS SFGMEWVRQA PEKGLEWVAY ISSDSSAIYY | 60 |
| Anti-B7-H3 | ADTVKGRFTI SRDNPKNTLF LQMTSLRSED TAMYYCGRGR ENIYYGSRLD YWGQGTTLTV | 120 |
| antibody | SS | 122 |
| hBRCA84D | | |
| variable heavy | | |
| chain | | |
| SEQ ID NO: 551 | DIAMTQSQKF MSTSVGDRVS VTCKASQNVD TNVAWYQQKP GQSPKALIYS ASYRYSGVPD | 60 |
| Anti-B7-H3 | RFTGSGSGTD FTLTINNVQS EDLAEYFCQQ YNNYPFTFGS GTKLEIK | 107 |
| antibody | | |
| hBRCA84D | | |
| variable light | | |
| chain | | |

In an embodiment, an anti-187-H-3 scFv domain comprises a heavy chain variable region (V_{H}) domain and/or a light chain variable region (V_{L}) domain of the antibody BRCA84D (including BRCA84D-1 and BRCA84D-2 and their variants), BRCA68D, BRCA69D, PRCA157, TES7, OVCA22, GB8, or SG27, including humanized or murine variants, or variants, derivatives, fragments, or conservative amino acid substitutions thereof. In an embodiment, an anti-B7-H3 scFv domain comprises a V_{H} domain and a V_{L} domain, wherein the V_{H} domain comprises the sequence shown in SEQ ID NO:542, or conservative amino acid substitutions thereof, and the light chain variable region (V_{L}) comprises the sequence shown in SEQ ID NO:543, or conservative amino acid substitutions thereof. In an embodiment, an anti-B7-H3 scFv domain comprises V_{H} and V_{L} regions that are each at least 99% identical to the sequences shown in SEQ ID NO:542 and SEQ ID NO:543, respectively. In an embodiment, an anti-B7-H3 scFv domain comprises V_{H} and V_{L} regions that are each at least 98% identical to the sequences shown in SEQ ID NO:542 and SEQ ID NO:543, respectively. In an embodiment, an anti-B7-H3 scFv domain comprises V_{H} and V_{L} regions that are each at least 97% identical to the sequences shown in SEQ ID NO:542 and SEQ ID NO:543, respectively. In an embodiment, an anti-B7-H3 scFv domain comprises V_{H} and V_{L} regions that are each at least 96% identical to the sequences shown in SEQ ID NO:542 and SEQ ID NO:543, respectively. In an embodiment, an anti-B7-H3 scFv domain comprises V_{H} and V_{L} regions that are each at least 95% identical to the sequences shown in SEQ ID NO:542 and SEQ ID NO:543, respectively. In an embodiment, an anti-B7-H3 scFv domain comprises V_{H} and V_{L} regions that are each at least 90% identical to the sequences shown in SEQ ID NO:542 and SEQ ID NO:543, respectively. In an embodiment, an anti-B7-H3 scFv domain comprises V_{H} and V_{L} regions that are each at least 85% identical to the sequences shown in SEQ ID NO:542 and SEQ ID NO:543, respectively. In an embodiment, an anti-B7-H3 scFv domain comprises V_{H} and V_{L} regions that are each at least 80% identical to the sequences shown in SEQ ID NO:542 and SEQ ID NO:543, respectively.

In an embodiment, an anti-B7-H3 scFv domain comprises the heavy chain CDR1, CDR2 and CDR3 domains and the light chain CDR1, CDR2, and CDR3 domains, or conservative amino acid substitutions thereof, of the antibody BRCA84D (including BRCA84D-1 and BRCA84D-2 and their variants), BRCA68D, BRCA69D, PRCA157, TES7, OVCA22, GB8, or SG27, including both humanized or murine variants. In an embodiment, an anti-B7-H3 scFv domain comprises the heavy chain CDR1, CDR2 and CDR3 domains of having the sequences set forth in SEQ ID NO:544, SEQ ID NO:545, and/or SEQ ID NO:546, respectively, or conservative amino acid substitutions thereof, and/or light chain CDR1, CDR2 and CDR3 domains having the sequences set forth in SEQ ID NO:547, SEQ ID NO:548, and/or SEQ ID NO:549, respectively, or conservative amino acid substitutions thereof.

In an embodiment, an anti-B7-H3 scFv domain comprises a V_{H} domain and a V_{L} domain, wherein the V_{H} domain comprises the sequence shown in SEQ ID NO:550, or conservative amino acid substitutions thereof, and the light chain variable region (V_{L}) comprises the sequence shown in SEQ ID NO:551, or conservative amino acid substitutions thereof. In an embodiment, an anti-B7-H3 scFv domain comprises V_{H} and V_{L} regions that are each at least 99% identical to the sequences shown in SEQ ID NO:550 and SEQ ID NO:551, respectively. In an embodiment, an anti-B7-H3 scFv domain comprises V_{H} and V_{L} regions that are each at least 98% identical to the sequences shown in SEQ ID NO:550 and SEQ ID NO:551, respectively. In an embodiment, an anti-B7-H3 scFv domain comprises V_{H} and V_{L} regions that are each at least 97% identical to the sequences shown in SEQ ID NO:550 and SEQ ID NO:551, respectively. In an embodiment, an anti-B7-H3 scFv domain comprises V_{H} and V_{L} regions that are each at least 96% identical to the sequences shown in SEQ ID NO:550 and SEQ ID NO:551, respectively. In an embodiment, an anti-B7-H3 scFv domain comprises V_{H} and V_{L} regions that are each at least 95% identical to the sequences shown in SEQ ID NO:550 and SEQ ID NO:551, respectively. In an embodiment, an anti-B7-H3 scFv domain comprises V_{H} and V_{L} regions that are each at least 90% identical to the sequences shown in SEQ ID NO:550 and SEQ ID NO:551, respectively. In an embodiment, an anti-B7-H3 scFv domain comprises V_{H} and V_{L} regions that are each at least 85% identical to the sequences shown in SEQ ID NO:550 and SEQ ID NO:551, respectively. In an embodiment, an anti-B7-H3 scFv domain comprises V_{H} and V_{L} regions that are each at least 80% identical to the sequences shown in SEQ ID NO:550 and SEQ ID NO:551, respectively.

In an embodiment, an anti-B7-H3 scFv domain comprises a V_{H} domain and a V_{L} domain, wherein the V_{H} domain comprises a sequence selected from the group consisting of BRCA84D, BRCA69D, PRCA157, BRCA84D-1, hBRCA84D-2V_{H}, hBRCA84D-3V_{H}, hBRCA84D-4V_{H}, chBRCA84D, or conservative amino acid substitutions thereof, and the light chain variable region (V_{L}) comprises a sequence selected from the group consisting of BRCA84D, BRCA69D, PRCA157, BRCA84D-1, hBRCA84D-2V_{L}, hBRCA84D-3V_{L}, hBRCA84D-4V_{L}, hBRCA84D-5V_{L}, hBRCA84D-6V_{L}, chBRCA84D, or conservative amino acid substitutions thereof, each as disclosed in U.S. Pat. No. 10,730,945, the disclosures of which are incorporated by reference herein.

In an embodiment, the anti-B7-H3 binding domain includes an scFv, V_{H} and/or V_{L} sequence, or a heavy chain and/or a light chain CDR1, CDR2, and/or CDR3 sequence as disclosed in U.S. Pat. No. 9,371,395, the disclosures of which are incorporated by reference herein.

In an embodiment, the anti-B7-H3 binding domain includes an scFv, V_{H} and/or V_{L} sequence, or a heavy chain and/or a light chain CDR1, CDR2, and/or CDR3 sequence as disclosed in U.S. Pat. No. 10,501,544, the disclosures of which are incorporated by reference herein.

In an embodiment, the anti-B7-H3 binding domain includes an scFv, V_{H} and/or V_{L} sequence, or a heavy chain and/or a light chain CDR1, CDR2, and/or CDR3 sequence as disclosed in U.S. Patent Application Publication No. US 2020/0338209 A1, the disclosures of which are incorporated by reference herein.

### 13. Extracellular DLL3 Binding Domains

In an embodiment, a CCR comprises an extracellular domain, wherein the extracellular domain is a domain capable of binding to human delta-like ligand 3 (DLL3). In an embodiment, the extracellular domain binds to human DLL3, also known as fibroblast activation protein and fibroblast activating protein a. DLL3 is known as a biomarker in SCLC, which is expressed 80% in SCLC while not seen in normal lung cells. DLL3 is an inhibitory ligand belonging to the notch signaling pathway. DLL3 inhibits the Notch signaling pathway in SCLC and other neuroendocrine tumors. The function of DLL3 and other DLL family members and their role and expression in solid tumors, such as small cell lung cancer (SCLC) and non-small cell lung cancer (NSCLC), or prostate cancer, are described in Owen D H, et al., J Hematol Oncol. 2019 Jun. 18; 12(1):61; and Xiu M X et al., Onco Targets Ther. 2020 May 7; 13:3881-3901, the disclosure of each of which are incorporated by reference herein. In an embodiment, the extracellular DLL3 binding domain is a scFv domain. In an embodiment, the DLL3 scFv binding domain binds to murine DLL3. In an embodiment, the DLL3 scFv binding domain binds to human DLL3. In an embodiment, a CCR of the present invention comprises a construct as shown in FIG. 34, wherein the V_{H} and V_{L} domains are anti DLL3 V_{H} and V_{L} domains, and the linker is as described herein.

In an embodiment, a CCR of the present invention comprises an extracellular domain, wherein the extracellular domain comprises a DLL3 binding domain. In an embodiment, the DLL3 binding domain is an anti-DLL3 binding domain described in U.S. Pat. Nos. 8,986,972; 9,089,615; 9,089,616; 9,089,617; 9,770,518 the disclosure of each of which are incorporated by reference herein. Other suitable anti-DLL3 binding domains known in the art may also be used (see for instance, genome.jp/dbget-bin/www_bget?dr:D11117).

In an embodiment, the anti DLL3 binding domain includes scFv, VH and/or VL sequences or heavy chain and/or light chain CDR1, CDR2, and/or CDR3 sequences of anti DLL3 antibody also known as rovalpituzumab or Rova (with for instance a chemical formula of: C6416H9894N1698O2028S46). In some embodiments, an antibody-drug conjugate such as rovalpituzumab tesirine (Rova-T) can be used to specifically bind to DLL3 in patients with SCLC.

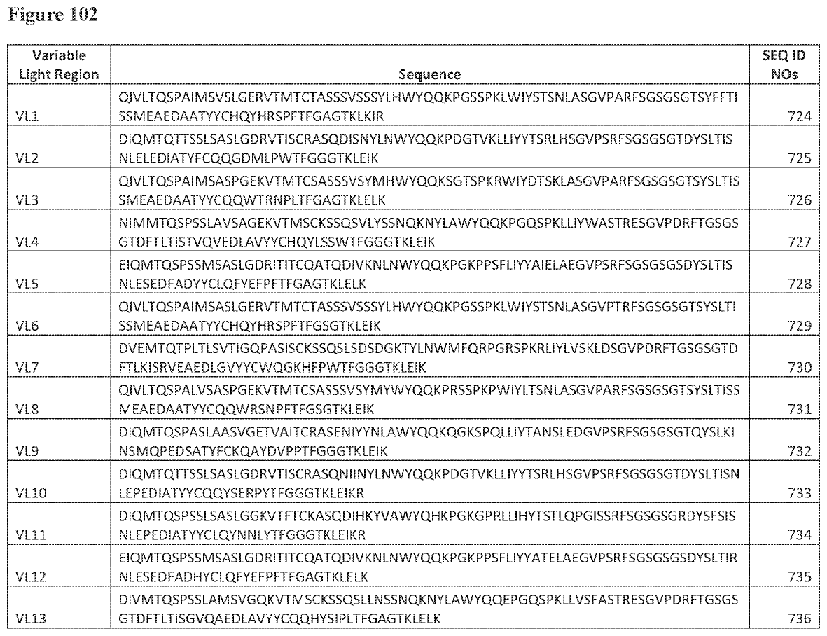

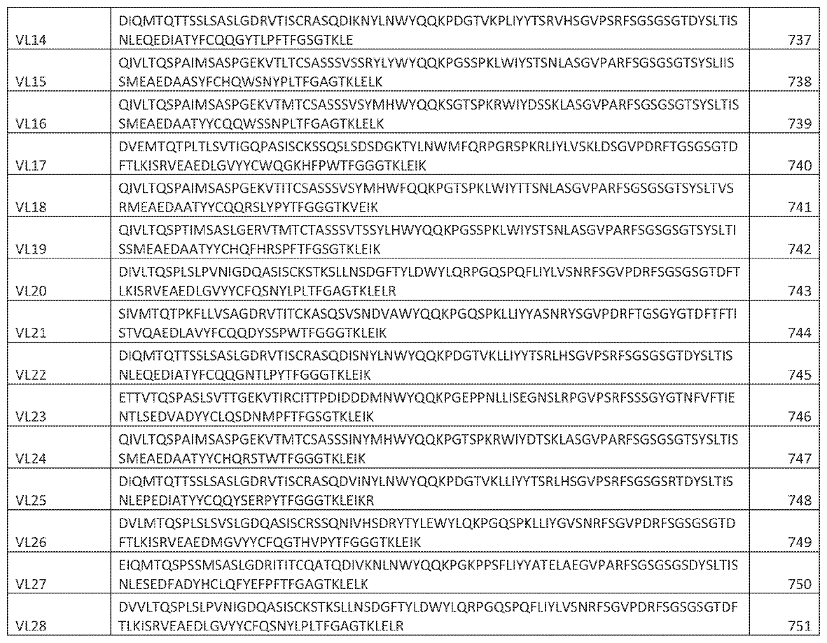

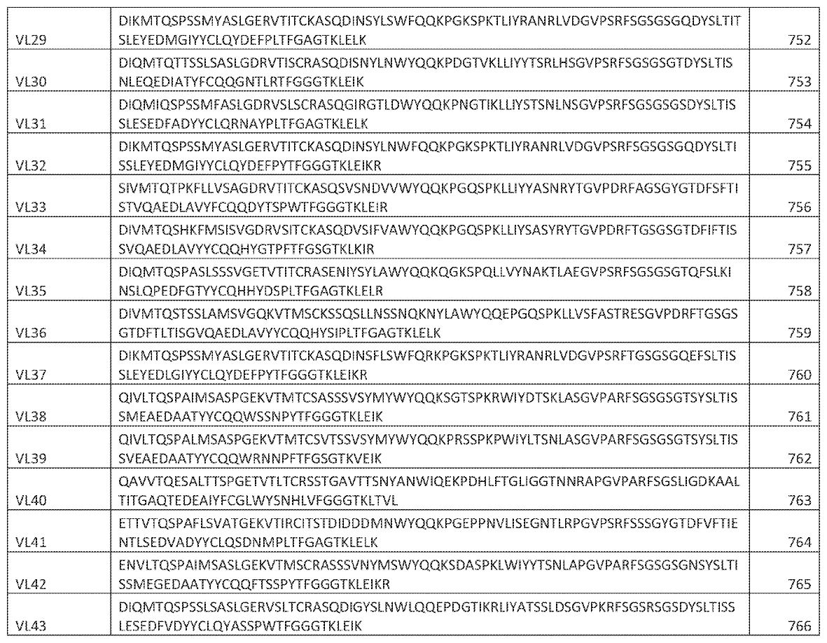

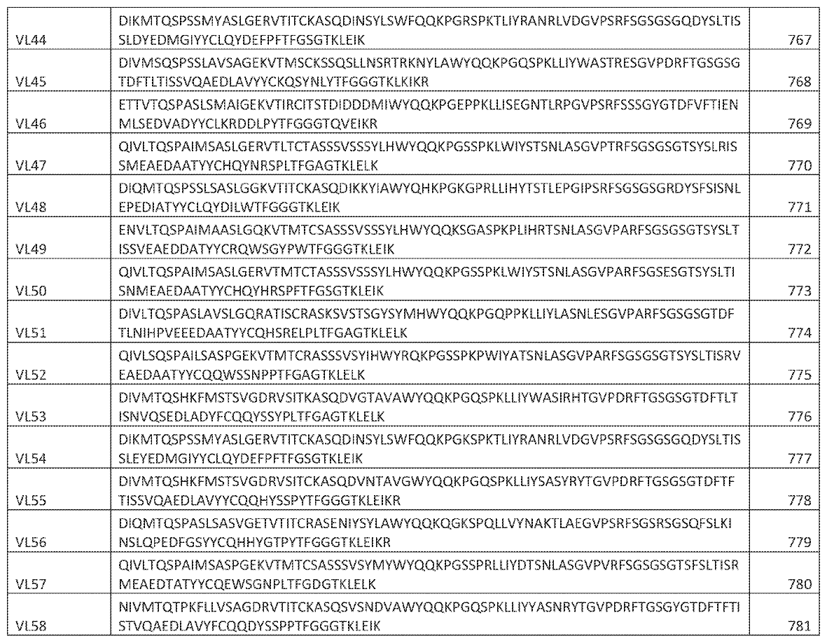

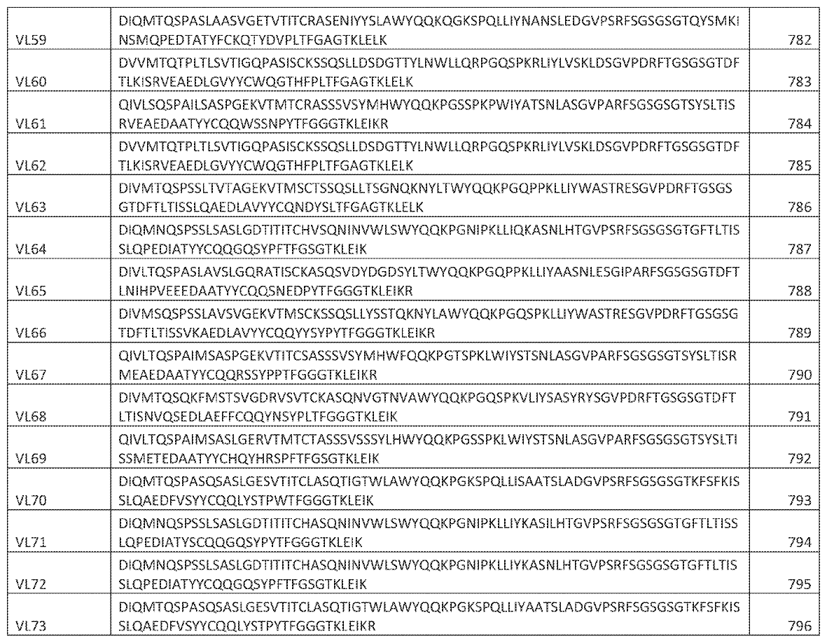

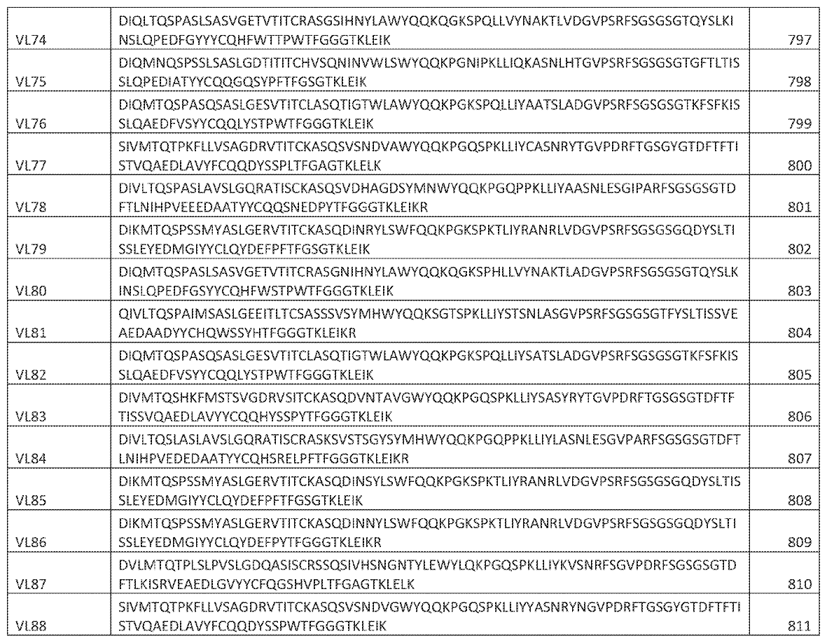

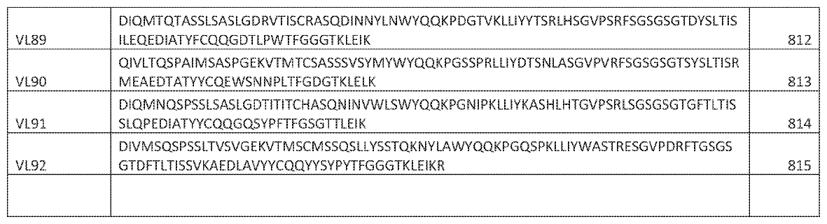

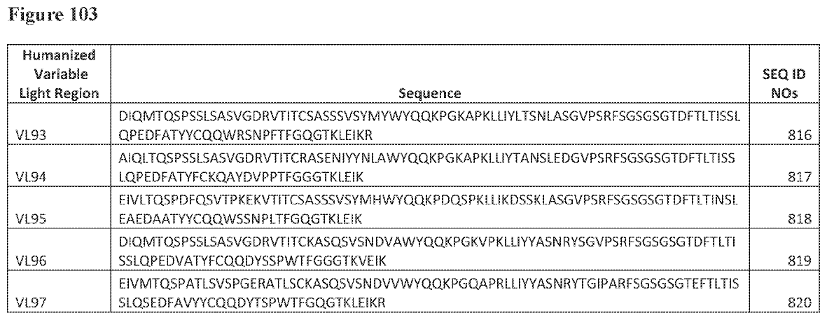

In an embodiment, the anti DLL3 binding domain includes a VH and/or VL sequence or a heavy chain and/or a light chain CDR1, CDR2, and/or CDR3 sequence, or a nucleotide encoding such a sequence. The amino acid sequences of exemplary DLL3 binding scFv domains are provided in FIGS. 102-105, and in Table 98.

TABLE 98
Amino acid sequences of
exemplary DLL3 binding scFv domains.

| | Sequence |
|---|---|
| Identifier | (One-Letter Amino Acid Symbols) |
| SEQ ID NO: 918 | QVQLVQSGAE VKKPGASVKV SCKASGYTFT |
| Anti-DLL3 | NYGMNWVRQA PGQGLEWMGW INTYTGEPTY |
| antibody | ADDFKGRVTM TTDTSTSTAY MELRSLRSDD |
| variable heavy | TAVYYCARIG DSSPSDYWGQ GTLVTVSSAS |
| chain (VH) | TKGPSVFPLA PSSKSTSGGT AALGCLVKDY |
| | FPEPVTVSWN SGALTSGVHT FPAVLQSSGL |
| | YSLSSVVTVP SSSLGTQTYI CNVNHKPSNT |
| | KVDKKVEPKS CDKTHTCPPC PAPELLGGPS |
| | VFLFPPKPKD TLMISRTPEV TCVVVDVSHE |
| | DPEVKFNWYV DGVEVHNAKT KPREEQYNST |
| | YRVVSVLTVL HQDWLNGKEY KCKVSNKALP |
| | APIEKTISKA KGQPREPQVY TLPPSRDELT |
| | KNQVSLTCLV KGFYPSDIAV EWESNGQPEN |
| | NYKTTPPVLD SDGSFFLYSK LTVDKSRWQQ |
| | GNVFSCSVMH EALHNHYTQK SLSLSPG |
| SEQ ID NO: 919 | EIVMTQSPAT LSVSPGERAT LSCKASQSVS |
| Anti-DLL3 | NDVVWYQQKP GQAPRLLIYY ASNRYTGIPA |
| antibody | RFSGSGSGTE FTLTISSLQS EDFAVYYCQQ |
| variable light | DYTSPWTFGQ GTKLEIKRTV AAPSVFIFPP |
| chain (VL) | SDEQLKSGTA SVVCLLNNFY PREAKVQWKV |
| | DNALQSGNSQ ESVTEQDSKD STYSLSSTLT |
| | LSKADYEKHK VYACEVTHQG LSSPVTKSFN |
| | RGEC |

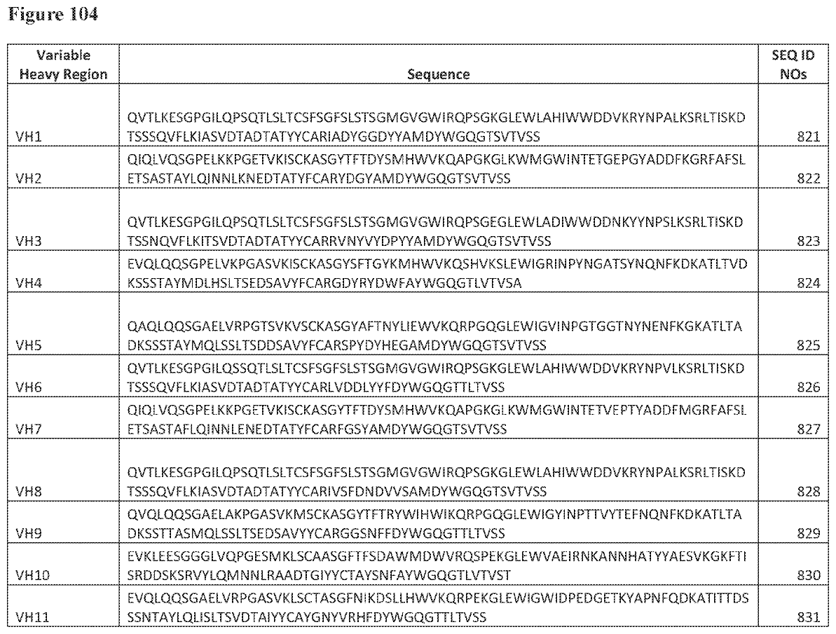

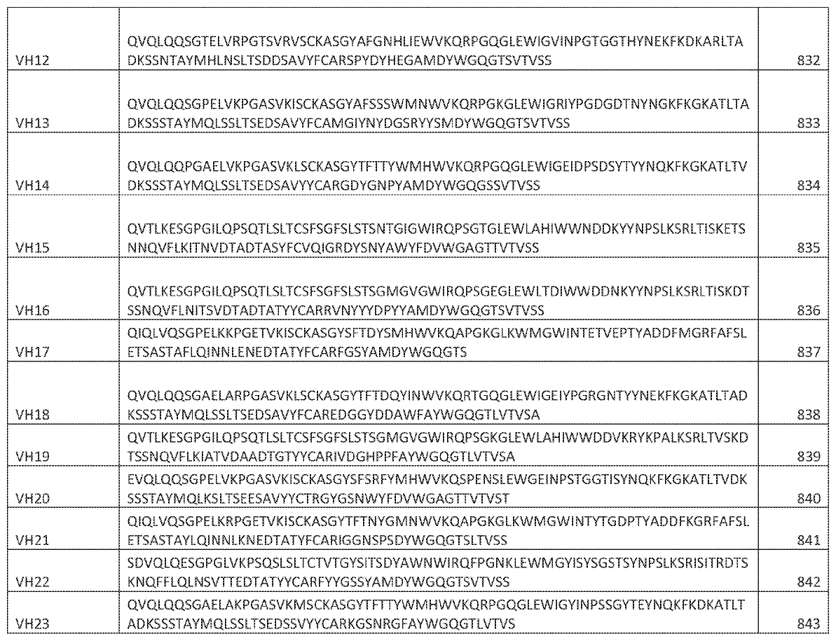

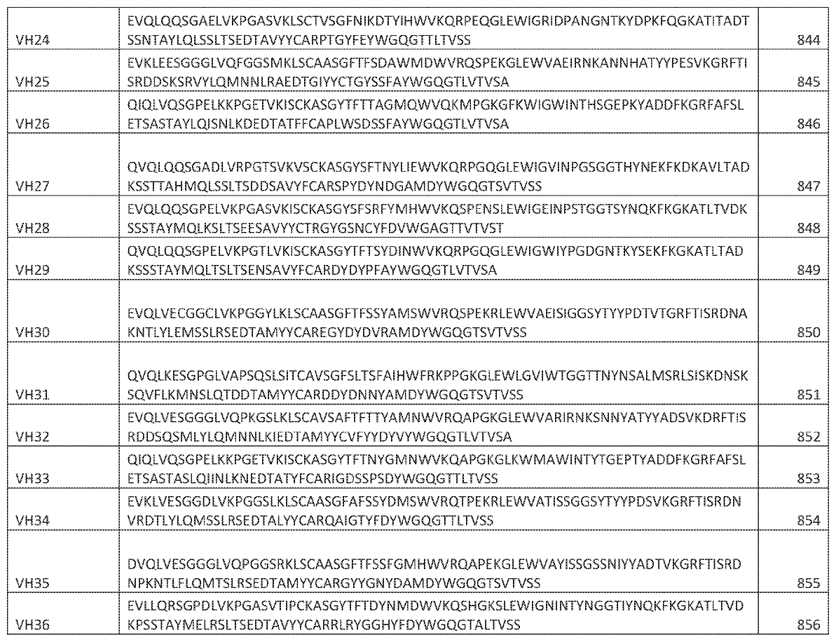

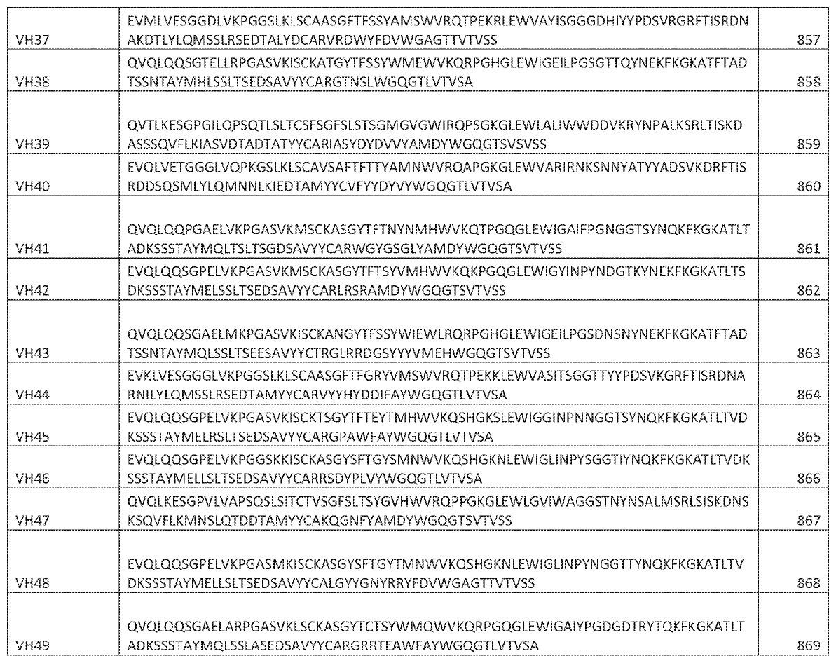

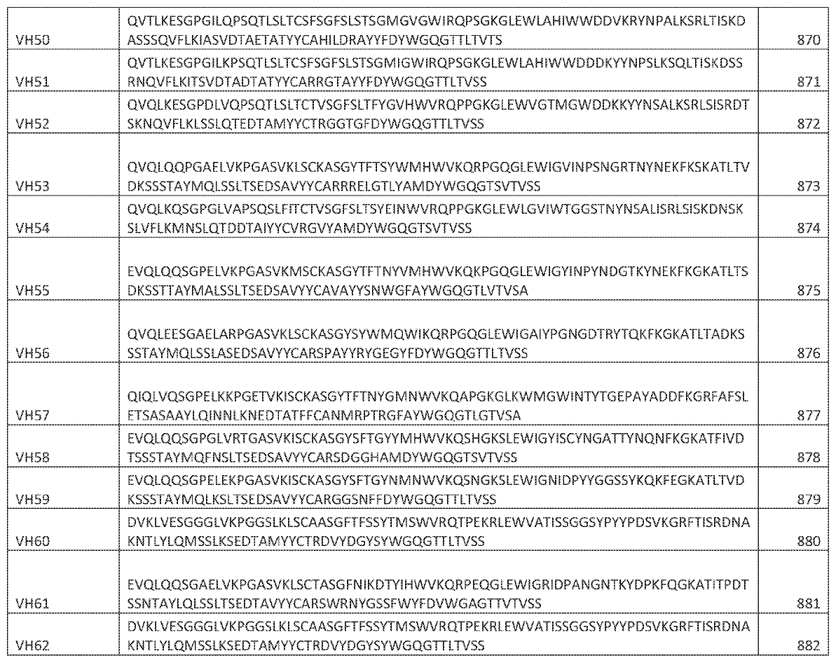

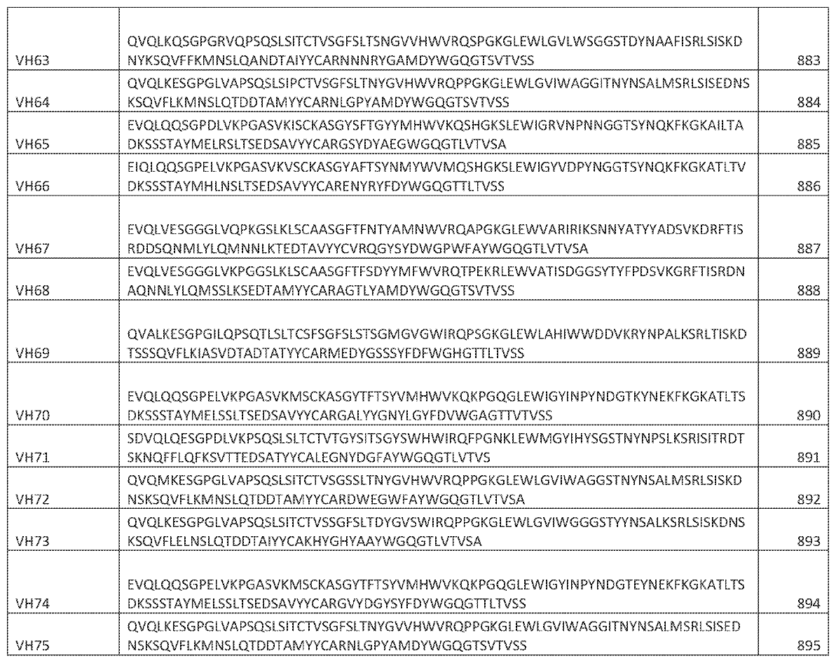

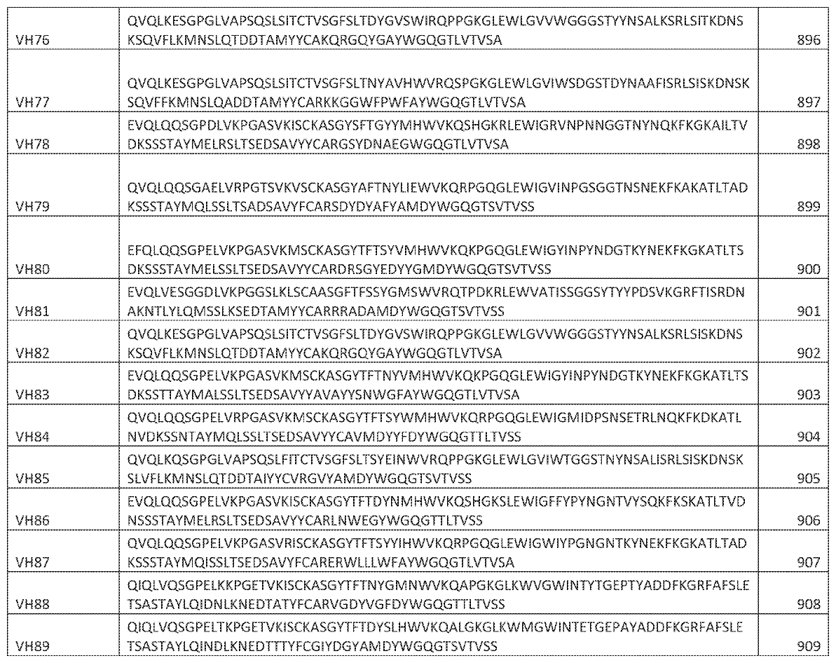

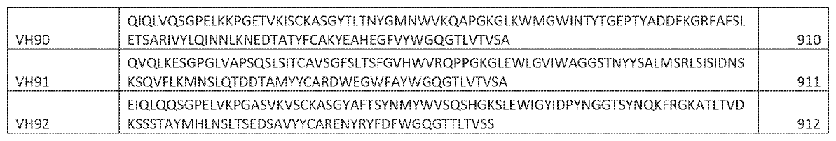

The amino acid sequences of exemplary DLL3 binding scFv domains are provided in FIGS. 102 and 104 which provide the amino acid sequences for DLL3 antibody variable light chain region (VL1-VL92) comprising FR1, CDR1, FR2, CDR2, FR3, and CDR3 (SEQ ID NOs: 816-815) and in respective order, their corresponding amino acid sequences for DLL3 antibody variable heavy chain region (VH1-VH92) comprising FR1, CDR1, FR2, CDR2, FR3, and CDR3 (SEQ ID NOs: 821-912). The amino acid sequences of exemplary DLL3 binding scFv domains are provided in Table 98 which provides the amino acid sequence for DLL3 antibody variable light chain region (SEQ ID NO:919) and for DLL3 antibody variable light chain region (SEQ ID NO:918).

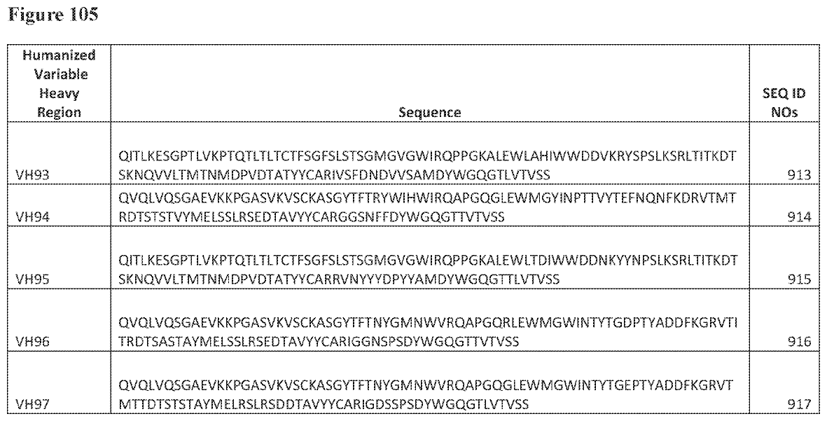

The amino acid sequences of exemplary humanized DLL3 binding scFv domains are provided in FIGS. 103 and 105 which provide the amino acid sequences for humanized DLL3 antibody variable light chain region (VL93-VL97) comprising FR1, CDR1, FR2, CDR2, FR3, and CDR3 (SEQ ID NOs: 816-820) and in respective order, their corresponding amino acid sequences for humanized DLL3 antibody variable heavy chain region (VH93-VH97) comprising FR1, CDR1, FR2, CDR2, FR3, and CDR3 (SEQ ID NOs: 913-917).

In an embodiment, an anti DLL3 scFv domain comprises the sequences shown in SEQ ID NOs: 724-815, 821-912, and 918-919, or conservative amino acid substitutions thereof. In an embodiment, an anti DLL3 scFv domain comprises a scFv domain that is at least 99% identical to the sequence shown in SEQ ID NOs: 724-815, 821-912, and 918-919. In an embodiment, an anti DLL3 scFv domain comprises a scFv domain that is at least 98% identical to the sequence shown in SEQ ID NOs: 724-815, 821-912, and 918-919. In an embodiment, an anti DLL3 scFv domain comprises a scFv domain that is at least 97% identical to the sequence shown in SEQ ID NOs: 724-815, 821-912, and 918-919. In an embodiment, an anti DLL3 scFv domain comprises a scFv domain that is at least 96% identical to the sequence shown in SEQ ID NOs: 724-815, 821-912, and 918-919. In an embodiment, an anti DLL3 scFv domain comprises a scFv domain that is at least 95% identical to the sequence shown in SEQ ID NOs: 724-815, 821-912, and 918-919. In an embodiment, an anti DLL3 scFv domain comprises a scFv domain that is at least 90% identical to the sequence shown in SEQ ID NOs: 724-815, 821-912, and 918-919. In an embodiment, an anti DLL3 scFv domain comprises a scFv domain that is at least 85% identical to the sequence shown in SEQ ID NOs: 724-815, 821-912, and 918-919. In an embodiment, an anti DLL3 scFv domain comprises a scFv domain that is at least 80% identical to the sequence shown in SEQ ID NOs: 724-815, 821-912, and 918-919.

In an embodiment, a humanized anti DLL3 scFv domain comprises the sequences shown in SEQ ID NOs: 816-820 and 913-917, or conservative amino acid substitutions thereof. In an embodiment, a humanized anti DLL3 scFv domain comprises a scFv domain that is at least 99% identical to the sequence shown in SEQ ID NOs: 816-820 and 913-917. In an embodiment, a humanized anti DLL3 scFv domain comprises a scFv domain that is at least 98% identical to the sequence shown in SEQ ID NOs: 816-820 and 913-917. In an embodiment, a humanized anti DLL3 scFv domain comprises a scFv domain that is at least 97% identical to the sequence shown in SEQ ID NOs: 816-820 and 913-917. In an embodiment, a humanized anti DLL3 scFv domain comprises a scFv domain that is at least 96% identical to the sequence shown in SEQ ID NOs: 816-820 and 913-917. In an embodiment, a humanized anti DLL3 scFv domain comprises a scFv domain that is at least 95% identical to the sequence shown in SEQ ID NOs: 816-820 and 913-917. In an embodiment, a humanized anti DLL3 scFv domain comprises a scFv domain that is at least 90% identical to the sequence shown in SEQ ID NOs: 816-820 and 913-917. In an embodiment, a humanized anti DLL3 scFv domain comprises a scFv domain that is at least 85% identical to the sequence shown in SEQ ID NOs: 816-820 and 913-917. In an embodiment, a humanized anti DLL3 scFv domain comprises a scFv domain that is at least 80% identical to the sequence shown in SEQ ID NOs: 816-820 and 913-917.

In an embodiment, an anti DLL3 scFv domain comprises a heavy chain variable region (VH) domain and/or a light chain variable region (VL) domain, wherein the VH domain comprises the sequence shown in SEQ ID NOs: 821-912 and 918, or conservative amino acid substitutions thereof, and the light chain variable region (VL) comprises the sequence shown in SEQ ID NOs: 724-815 and 919, or conservative amino acid substitutions thereof. In an embodiment, an anti DLL3 scFv domain comprises VH and VL regions that are each at least 99% identical to the sequences shown in SEQ ID NOs: 821-912 and 918, and SEQ ID NOs: 724-815 and 919, respectively. In an embodiment, an anti DLL3 scFv domain comprises VH and VL regions that are each at least 98% identical to the sequences shown in SEQ ID NOs: 821-912 and 918, and SEQ ID NOs: 724-815 and 919, respectively. In an embodiment, an anti DLL3 scFv domain comprises VH and VL regions that are each at least 97% identical to the sequences shown in SEQ ID NOs: 821-912 and 918, and SEQ ID NOs: 724-815 and 919, respectively. In an embodiment, an anti DLL3 scFv domain comprises VH and VL regions that are each at least 96% identical to the sequences shown in SEQ ID NOs: 821-912 and 918, and SEQ ID NOs: 724-815 and 919, respectively. In an embodiment, an anti DLL3 scFv domain comprises VH and VL regions that are each at least 95% identical to the sequences shown in SEQ ID NOs: 821-912 and 918, and SEQ ID NOs: 724-815 and 919, respectively. In an embodiment, an anti DLL3 scFv domain comprises VH and VL regions that are each at least 90% identical to the sequences shown in SEQ ID NOs: SEQ ID NOs: 821-912 and 918, and SEQ ID NOs: 724-815 and 919, respectively. In an embodiment, an anti DLL3 scFv domain comprises VH and VL regions that are each at least 85% identical to the sequences shown in SEQ ID NOs: 821-912 and 918, and SEQ ID NOs: 724-815 and 919, respectively. In an embodiment, an anti DLL3 scFv domain comprises VH and VL regions that are each at least 80% identical to the sequences shown in SEQ ID NOs: 821-912 and 918, and SEQ ID NOs: 724-815 and 919, respectively.

In an embodiment, a humanized anti DLL3 scFv domain comprises a heavy chain variable region (VH) domain and/or alight chain variable region (VL) domain, wherein the VH domain comprises the sequence shown in SEQ ID NOs: 913-917, or conservative amino acid substitutions thereof, and the light chain variable region (VL) comprises the sequence shown in SEQ ID NOs: 816-820, or conservative amino acid substitutions thereof. In an embodiment, a humanized anti DLL3 scFv domain comprises VH and VL regions that are each at least 99% identical to the sequences shown in SEQ ID NOs: 913-917 and SEQ ID NOs: 816-820, respectively. In an embodiment, a humanized anti DLL3 scFv domain comprises VH and VL regions that are each at least 98% identical to the sequences shown in SEQ ID NOs: 913-917 and SEQ ID NOs: 816-820, respectively. In an embodiment, a humanized anti DLL3 scFv domain comprises VH and VL regions that are each at least 97% identical to the sequences shown in SEQ ID NOs: 913-917 and SEQ ID NOs: 816-820, respectively. In an embodiment, a humanized anti DLL3 scFv domain comprises VH and VL regions that are each at least 96% identical to the sequences shown in SEQ ID NOs: 913-917 and SEQ ID NOs: 816-820, respectively. In an embodiment, a humanized anti DLL3 scFv domain comprises VH and VL regions that are each at least 95% identical to the sequences shown in SEQ ID NOs: 913-917 and SEQ ID NOs: 816-820, respectively. In an embodiment, a humanized anti DLL3 scFv domain comprises VH and VL regions that are each at least 90% identical to the sequences shown in SEQ ID NOs: 913-917 and SEQ ID NOs: 816-820, respectively. In an embodiment, a humanized anti DLL3 scFv domain comprises VH and VL regions that are each at least 85% identical to the sequences shown in SEQ ID NOs: 913-917 and SEQ ID NOs: 816-820, respectively. In an embodiment, a humanized anti DLL3 scFv domain comprises VH and VL regions that are each at least 80% identical to the sequences shown in SEQ ID NOs: 913-917 and SEQ ID NOs: 816-820, respectively.

In an embodiment, a nucleotide sequence encoding anti DLL3 scFv domain is codon-optimized to improve protein expression. In an embodiment, including the foregoing embodiments, a nucleotide sequence encoding anti DLL3 scFv domain comprising SEQ ID NOs: 821-912 and 918, and SEQ ID NOs: 724-815 and 919, and/or SEQ ID NOs: 816-820 and 913-917 is/are codon-optimized to improve protein expression.

### 14. Other Extracellular Binding Domains

In an embodiment, a CCR of the present invention comprises an extracellular domain, wherein the extracellular domain comprises a CD44 binding domain. In an embodiment, the anti-CD44 binding domain includes a scFv, V_{H} and/or V_{L} sequence or a heavy chain and/or a light chain CDR1, CDR2, and/or CDR3 sequence as disclosed in U.S. Pat. Nos. 7,361,347; 9,388,249 and 11,220,544 and in U.S. Patent Application Publication Nos. US 2004/0048319 A1; US 2005/0214283 A1; US 2007/0237761 A1; US 2010/0092484 A1; US 2012/0201751 A1; and US 2020/0291113 A1, the disclosures of each of which are incorporated by reference herein. Other suitable anti-CD44 binding domains known in the art may also be used.

In an embodiment, a CCR of the present invention comprises an extracellular domain, wherein the extracellular domain comprises a CD40 binding domain. In an embodiment, the anti-CD40 binding domain includes a scFv, V_{H} and/or V_{L} sequence or a heavy chain and/or a light chain CDR1, CDR2, and/or CDR3 sequence as disclosed in International Patent Publication No. WO 2018/027025 A1; U.S. Patent Publication Nos. 2021/0188992 A1 and 2015/0110783 A1; and U.S. Pat. Nos. 11,001,637 and 10,577,425; the disclosures of each of which are incorporated by reference herein. CD40 binding domains also include the binding or extracellular portions of CD40 ligand (CD40L) domains. Other suitable anti-CD40 binding domains known in the art may also be used.

In an embodiment, a CCR of the present invention comprises an extracellular domain, wherein the extracellular domain comprises an ALCAM (CD166) binding domain. In an embodiment, the anti-ALCAM (anti-CD166) binding domain includes a scFv, V_{H} and/or V_{L} sequence or a heavy chain and/or a light chain CDR1, CDR2, and/or CDR3 sequence as disclosed in U.S. Pat. Nos. 9,388,249 and 11,220,544 and in U.S. Patent Application Publication Nos. US 2004/0048319 A1 and US 2020/0291113 A1, the disclosures of each of which are incorporated by reference herein. Other suitable anti-ALCAM binding domains known in the art may also be used.

In an embodiment, a CCR of the present invention comprises an extracellular domain, wherein the extracellular domain comprises an IL-13Rα binding domain. In an embodiment, a CCR of the present invention comprises an extracellular domain, wherein the extracellular domain comprises an IL-13Rα1 binding domain. In an embodiment, a CCR of the present invention comprises an extracellular domain, wherein the extracellular domain comprises an IL-13Rα2 binding domain. In an embodiment, the anti-IL-13Rα binding domain includes a scFv, V_{H} and/or V_{L} sequence or a heavy chain and/or a light chain CDR1, CDR2, and/or CDR3 sequence as disclosed in U.S. Pat. Nos. 6,428,788; 7,994,302; 8,221,755; 9,315,575; 8,318,910; 9,650,438; 9,828,428; and 9,587,026; and in U.S. Patent Application Publication Nos. US 2019/0008966 A1; US 2021/0000875 A1; and US 2019/0359723 A1, the disclosures of each of which are incorporated by reference herein. Other suitable anti-IL-13Rα, IL-13Rα1, or IL-13Rα2 binding domains known in the art may also be used.

In an embodiment, a CCR of the present invention comprises an extracellular domain, wherein the extracellular domain comprises a transforming growth factor f receptor (TGFβR) binding domain. In an embodiment, a CCR of the present invention comprises an extracellular domain, wherein the extracellular domain comprises a TGFβRII (also referred to herein as a TGFβR2) binding domain. In an embodiment, the anti-TGFβR binding domain includes a scFv, V_{H} and/or V_{L} sequence or a heavy chain and/or a light chain CDR1, CDR2, and/or CDR3 sequence as disclosed in U.S. Pat. Nos. 6,201,108 and 7,579,186; in U.S. Patent Application Publication No. US 2012/0177666 A1; and in International Patent Application Publication No. WO 2021/133167 A1, the disclosures of each of which are incorporated by reference herein. TGFβR binding domains also include the binding or extracellular portions of TGFβ domains. Other suitable anti-TGFβR binding domains known in the art may also be used.

In an embodiment, a CCR of the present invention comprises an extracellular domain, wherein the extracellular domain comprises a transforming growth factor β (TGFβ) binding domain. In an embodiment, the anti-TGFβ binding domain includes a scFv, V_{H} and/or V_{L} sequence or a heavy chain and/or a light chain CDR1, CDR2, and/or CDR3 sequence as disclosed in U.S. Pat. Nos. 10,947,303; 9,714,285; and 9,676,863 and in U.S. Patent Application Publication No. US 2021/0061897 A1, the disclosures of each of which are incorporated by reference herein. TGFβ binding domains also include the binding or extracellular portions of TGFβR domains, including the TGFβRII domain. Other suitable anti-TGFβ binding domains known in the art may also be used.

In an embodiment, a CCR of the present invention comprises an extracellular domain, wherein the extracellular domain comprises an FAS (or Fas) binding domain. In an embodiment, the anti-FAS binding domain includes a scFv, V_{H} and/or V_{L} sequence or a heavy chain and/or a light chain CDR1, CDR2, and/or CDR3 sequence as disclosed in U.S. Pat. Nos. 6,086,877; 6,746,673; and 6,972,323; and U.S. Patent Application Publication Nos. US 2006/0083738 A1 and US 2010/0233157 A1, the disclosures of each of which are incorporated by reference herein. Other suitable anti-FAS binding domains known in the art may also be used.

In an embodiment, any of the extracellular domains disclosed herein may be used to create biepitope binding CCR constructs, as described elsewhere herein.

### B. Transmembrane and Hinge Domains

In an embodiment, a CCR comprises a transmembrane domain. In an embodiment, the transmembrane domain is linked at one end to an extracellular domain of the CCR and at the other end to at least one intracellular domain of the CCR. In an embodiment, the transmembrane domain is linked at one end to an extracellular domain of the CCR and at the other end to at least one intracellular domain of the CCR. In another embodiment, a CCR is designed to comprise a transmembrane domain that is fused to a spacer or hinge domain of the CCR, which is itself fused to the extracellular domain of the CCR. In an embodiment, the transmembrane domain that naturally is associated with one of the domains in the CCR is used. In some embodiments, the transmembrane domain can be selected or modified by amino acid substitution to avoid binding of such domains to the transmembrane domains of the same or different surface membrane proteins, for example, without being bound by any theory, to minimize interactions with other members of the receptor complex.

Transmembrane domains of use with the CCRs of the present invention may be derived from or comprise at least the transmembrane region(s) of the alpha (α), beta (β), or zeta (ζ) chain of the T-cell receptor (including CD3ζ), CD3 epsilon (CD3ε), CD4, CD5, CD8 (including CD8α), CD9, CD16, CD22, CD27, CD28, CD33, CD37, CD45, CD64, CD80, CD86, CD134, CD137, CD154, IgG1, IgG4, IgD, IL-2Rα, IL-2Rβ, and IL-2Rγ. In some embodiments, the transmembrane domain may be synthetic, and comprise predominantly hydrophobic residues such as leucine and valine. In some embodiments, a triplet of phenylalanine, tryptophan, and valine will be positioned at each end of a synthetic transmembrane domain. Optionally, in some embodiments, a short oligo- or polypeptide linker, between 2 and 10 amino acids in length, may form the linkage between the transmembrane domain and the intracellular domain of the CCR, and in some embodiments, this linker may comprise a glycine-serine doublet, as described elsewhere herein. Suitable, non-limiting transmembrane domains useful in the CCR constructs of the present invention are set forth in Table 56.

TABLE 56
Amino acid sequences of exemplary transmembrane and hinge domains.

| Identifier | Sequence (One-Letter Amino Acid Symbols) | |
|---|---|---|
| SEQ ID NO: 552 | VGVVGGLLGS LVLLVWVLAV I | 21 |
| PD-1 | | |
| transmembrane | | |
| domain | | |
| SEQ ID NO: 553 | FWVLVVVGGV LACYSLLVTV AFIIFWV | 27 |
| CD28 | | |
| transmembrane | | |
| domain | | |
| SEQ ID NO: 554 | ILVIFSGMFL VFTLAGALFL H | 21 |
| CD27 | | |
| transmembrane | | |
| domain | | |
| SEQ ID NO: 555 | IYIWAPLAGT CGVLLLSLVI TLYC | 24 |
| CD8α | | |
| transmembrane | | |
| domain | | |
| SEQ ID NO: 556 | TTTPAPRPPT PAPTIASQPL SLRPEACRPA AGGAVHTRGL DFACD | 45 |
| CD8α hinge | | |
| domain | | |
| SEQ ID NO: 557 | IPWLGHLLVG LSGAFGFIIL VYLLI | 25 |
| IL-2Rβ | | |
| transmembrane | | |
| domain | | |
| SEQ ID NO: 558 | EPKSCDKTHT CPPCPAPELL GGPSVFLFPP K | 31 |
| IgG1 | | |
| transmembrane | | |
| and hinge | | |
| domain | | |
| SEQ ID NO: 559 | EPKSCDKTHT CPPCPAPELL GGP | 23 |
| IgG1 hinge | | |
| domain | | |
| SEQ ID NO: 560 | ESKYGPPCPP CPAPEFLGGP SVFLFPPKPK DTLMISRTPE VTCVVVDVSQ EDPEVQFNWY | 60 |
| IgG4 hinge | VDGVEVHNAK TKPREEQFNS TYRVVSVLTV LHQDWLNGKE YKCKVSNKGL PSSIEKTISK | 120 |
| domain | AKGQPREPQV YTLPPSQEEM TKNQVSLTCL VKGFYPSDIA VEWESNGQPE NNYKTTPPVL | 180 |
| | DSDGSFFLYS RLTVDKSRWQ EGNVFSCSVM HEALHNHYTQ KSLSLSLGKM | 230 |
| SEQ ID NO: 561 | RWPESPKAQA SSVPTAQPQA EGSLAKATTA PATTRNTGRG GEEKKKEKEK EEQEERETKT | 60 |
| IgD hinge | PECPSHTQPL GVYLLTPAVQ DLWLRDKATF TCFVVGSDLK DAHLTWEVAG KVPTGGVEEG | 120 |
| domain | LLERHSNGSQ SQHSRLTLPR SLWNAGTSVT CTLNHPSLPP QRLMALREPA AQAPVKLSLN | 180 |
| | LLASSDPPEA ASWLLCEVSG FSPPNILLMW LEDQREVNTS GFAPARPPPQ PGSTTFWAWS | 240 |
| | VLRVPAPPSP QPATYTCVVS HEDSRTLLNA SRSLEVSYVT DH | 282 |

In an embodiment, a CCR of the present invention includes a transmembrane domain and optionally a hinge domain. In an embodiment, a CCR of the present invention includes a transmembrane domain and optionally a hinge domain, as shown in FIG. 34. In an embodiment, a CCR of the present invention includes a transmembrane domain selected from the transmembrane regions of the group consisting of the alpha (α), beta (β), or zeta (ζ) chain of the T-cell receptor (including CD3ζ), CD3 epsilon (CD3ε), CD4, CD5, CD8 (including CD8α), CD9, CD16, CD22, CD27, CD28, CD33, CD37, CD45, CD64, CD80, CD86, CD134, CD137, CD154, IgG1, IgG4, IgD, IL-2Rα, IL-2Rβ, IL-2Rγ, and variants, fragments, and derivatives thereof. In some embodiments, the transmembrane domain of CCRs of the present invention may be recombinant, in which case it will comprise predominantly hydrophobic residues such as leucine and valine, and in some embodiments, a triplet of phenylalanine, tryptophan and valine may be located at each end of a recombinant transmembrane domain. In an embodiment, a CCR comprises a hinge domain. In an embodiment, the hinge domain is a spacer domain. In an embodiment, a CCR of the present invention comprises a hinge domain or a spacer domain derived from a human protein. In an embodiment, the hinge domain or spacer domain is located between, and linked to, an extracellular domain and a transmembrane domain. In some embodiments, a CCR of the present invention comprises an extracellular domain, a transmembrane domain, and an intracellular domain, without a hinge domain. In some embodiments, a CCR of the present invention comprises an extracellular domain, a hinge domain, a transmembrane domain, and an intracellular domain. In some embodiments, a CCR of the present invention comprises an extracellular domain and intracellular domain that includes a transmembrane domain, with or without a hinge domain.

In some instances, the transmembrane domain can be attached to the extracellular region of the CCR via a hinge domain, such as a hinge region from a human protein. For example, in some embodiments, CCRs of the present invention include a human Ig (immunoglobulin) hinge, such as, an IgG4 hinge, or a CD8α hinge. In an embodiment, a CCR of the present invention includes a hinge domain selected from the hinge regions of the group consisting of the alpha (α), beta (β), or zeta (ζ) chain of the T-cell receptor, CD3 epsilon (CD3ε), CD4, CD5, CD8, CD9, CD16, CD22, CD27, CD28, CD33, CD37, CD45, CD64, CD80, CD86, CD134, CD137, CD154, IgG1, IgG4, IgD, IL-2Rα, IL-2Rβ, IL-2Rγ, and variants, fragments, and derivatives thereof.

In an embodiment, a CCR of the present invention includes a PD-1 transmembrane domain comprising the amino acid sequence of SEQ ID NO:552, or conservative amino acid substitutions thereof, or fragments, variants, or derivatives thereof, or an amino acid sequence that is at least 99% identical to the sequence given in SEQ ID NO:552, at least 98% identical to the sequence given in SEQ ID NO:552, at least 97% identical to the sequence given in SEQ ID NO:552, at least 96% identical to the sequence given in SEQ ID NO:552, at least 95% identical to the sequence given in SEQ ID NO:552, at least 90% identical to the sequence given in SEQ ID NO:552, at least 85% identical to the sequence given in SEQ ID NO:552, or at least 80% identical to the sequence given in SEQ ID NO:552.

In an embodiment, a CCR of the present invention includes a CD28 transmembrane domain comprising the amino acid sequence of SEQ ID NO:553, or conservative amino acid substitutions thereof, or fragments, variants, or derivatives thereof, or an amino acid sequence that is at least 99% identical to the sequence given in SEQ ID NO:553, at least 98% identical to the sequence given in SEQ ID NO:553, at least 97% identical to the sequence given in SEQ ID NO:553, at least 96% identical to the sequence given in SEQ ID NO:553, at least 95% identical to the sequence given in SEQ ID NO:553, at least 90% identical to the sequence given in SEQ ID NO:553, at least 85% identical to the sequence given in SEQ ID NO:553, or at least 80% identical to the sequence given in SEQ ID NO:553.

In an embodiment, a CCR of the present invention includes a CD27 transmembrane domain comprising the amino acid sequence of SEQ ID NO:554, or conservative amino acid substitutions thereof, or fragments, variants, or derivatives thereof, or an amino acid sequence that is at least 99% identical to the sequence given in SEQ ID NO:554, at least 98% identical to the sequence given in SEQ ID NO:554, at least 97% identical to the sequence given in SEQ ID NO:554, at least 96% identical to the sequence given in SEQ ID NO:554, at least 95% identical to the sequence given in SEQ ID NO:554, at least 90% identical to the sequence given in SEQ ID NO:554, at least 85% identical to the sequence given in SEQ ID NO:554, or at least 80% identical to the sequence given in SEQ ID NO:554.

In an embodiment, a CCR of the present invention includes a CD8α transmembrane domain comprising the amino acid sequence of SEQ ID NO:555, or conservative amino acid substitutions thereof, or fragments, variants, or derivatives thereof, or an amino acid sequence that is at least 99% identical to the sequence given in SEQ ID NO:555, at least 98% identical to the sequence given in SEQ ID NO:555, at least 97% identical to the sequence given in SEQ ID NO:555, at least 96% identical to the sequence given in SEQ ID NO:555, at least 95% identical to the sequence given in SEQ ID NO:555, at least 90% identical to the sequence given in SEQ ID NO:555, at least 85% identical to the sequence given in SEQ ID NO:555, or at least 80% identical to the sequence given in SEQ ID NO:555.

In an embodiment, a CCR of the present invention includes a CD8α hinge domain comprising the amino acid sequence of SEQ ID NO:556, or conservative amino acid substitutions thereof, or fragments, variants, or derivatives thereof, or an amino acid sequence that is at least 99% identical to the sequence given in SEQ ID NO:556, at least 98% identical to the sequence given in SEQ ID NO:556, at least 97% identical to the sequence given in SEQ ID NO:556, at least 96% identical to the sequence given in SEQ ID NO:556, at least 95% identical to the sequence given in SEQ ID NO:556, at least 90% identical to the sequence given in SEQ ID NO:556, at least 85% identical to the sequence given in SEQ ID NO:556, or at least 80% identical to the sequence given in SEQ ID NO:556.

In an embodiment, a CCR of the present invention includes a IL-2Rβ transmembrane domain comprising the amino acid sequence of SEQ ID NO:557, or conservative amino acid substitutions thereof, or fragments, variants, or derivatives thereof, or an amino acid sequence that is at least 99% identical to the sequence given in SEQ ID NO:557, at least 98% identical to the sequence given in SEQ ID NO:557, at least 97% identical to the sequence given in SEQ ID NO:557, at least 96% identical to the sequence given in SEQ ID NO:557, at least 95% identical to the sequence given in SEQ ID NO:557, at least 90% identical to the sequence given in SEQ ID NO:557, at least 85% identical to the sequence given in SEQ ID NO:557, or at least 80% identical to the sequence given in SEQ ID NO:557.

In an embodiment, a CCR of the present invention includes an IgG1 transmembrane and hinge domain comprising the amino acid sequence of SEQ ID NO:558, or conservative amino acid substitutions thereof, or fragments, variants, or derivatives thereof, or an amino acid sequence that is at least 99% identical to the sequence given in SEQ ID NO:558, at least 98% identical to the sequence given in SEQ ID NO:558, at least 97% identical to the sequence given in SEQ ID NO:558, at least 96% identical to the sequence given in SEQ ID NO:558, at least 95% identical to the sequence given in SEQ ID NO:558, at least 90% identical to the sequence given in SEQ ID NO:558, at least 85% identical to the sequence given in SEQ ID NO:558, or at least 80% identical to the sequence given in SEQ ID NO:558.

In an embodiment, a CCR of the present invention includes an IgG1 hinge domain comprising the amino acid sequence of SEQ ID NO:559, or conservative amino acid substitutions thereof, or fragments, variants, or derivatives thereof, or an amino acid sequence that is at least 99% identical to the sequence given in SEQ ID NO:559, at least 98% identical to the sequence given in SEQ ID NO:559, at least 97% identical to the sequence given in SEQ ID NO:559, at least 96% identical to the sequence given in SEQ ID NO:559, at least 95% identical to the sequence given in SEQ ID NO:559, at least 90% identical to the sequence given in SEQ ID NO:559, at least 85% identical to the sequence given in SEQ ID NO:559, or at least 80% identical to the sequence given in SEQ ID NO:559.

In an embodiment, a CCR of the present invention includes an IgG4 hinge domain comprising the amino acid sequence of SEQ ID NO:560, or conservative amino acid substitutions thereof, or fragments, variants, or derivatives thereof, or an amino acid sequence that is at least 99% identical to the sequence given in SEQ ID NO:560, at least 98% identical to the sequence given in SEQ ID NO:560, at least 97% identical to the sequence given in SEQ ID NO:560, at least 96% identical to the sequence given in SEQ ID NO:560, at least 95% identical to the sequence given in SEQ ID NO:560, at least 90% identical to the sequence given in SEQ ID NO:560, at least 85% identical to the sequence given in SEQ ID NO:560, or at least 80% identical to the sequence given in SEQ ID NO:560.

In an embodiment, a CCR of the present invention includes an IgD hinge domain comprising the amino acid sequence of SEQ ID NO:561, or conservative amino acid substitutions thereof, or fragments, variants, or derivatives thereof, or an amino acid sequence that is at least 99% identical to the sequence given in SEQ ID NO:561, at least 98% identical to the sequence given in SEQ ID NO:561, at least 97% identical to the sequence given in SEQ ID NO:561, at least 96% identical to the sequence given in SEQ ID NO:561, at least 95% identical to the sequence given in SEQ ID NO:561, at least 90% identical to the sequence given in SEQ ID NO:561, at least 85% identical to the sequence given in SEQ ID NO:561, or at least 80% identical to the sequence given in SEQ ID NO:561.

The nucleotide sequences encoding exemplary transmembrane and hinge domains for use with CCRs of the present invention are provided in Table 57. In an embodiment, a nucleotide sequence in Table 57 is codon-optimized to improve protein expression.

TABLE 57
Nucleotide sequences of exemplary transmembrane and hinge domains.

| Identifier | Sequence (One-Letter Nucleotide Symbols) | |
|---|---|---|
| SEQ ID NO: 562 | GTTGGTGTCG TGGGCGGCCT GCTGGGCAGC CTGGTGCTGC TAGTCTGGGT CCTGGCCGTC | 60 |
| PD-1 | ATC | 63 |
| transmembrane | | |
| domain | | |
| SEQ ID NO: 563 | TTTTGGGTGC TGGTGGTGGT TGGTGGAGTC CTGGCTTGCT ATAGCTTGCT AGTAACAGTG | 60 |
| CD28 | GCCTTTATTA TTTTCTGGGT G | 81 |
| transmembrane | | |
| domain | | |
| SEQ ID NO: 564 | ATTCTGGTGA TTTTTAGCGG CATGTTTCTG GTGTTTACCC TGGCGGGCGC GCTGTTTCTG | 60 |
| CD27 | CAT | 63 |
| transmembrane | | |
| domain | | |
| SEQ ID NO: 565 | ATCTACATCT GGGCGCCCTT GGCCGGGACT TGTGGGGTCC TTCTCCTGTC ACTGGTTATC | 60 |
| CD8α | ACCCTTTACT GC | 72 |
| transmembrane | | |
| domain | | |
| SEQ ID NO: 566 | ACCACGACGC CAGCGCCGCG ACCACCAACA CCGGCGCCCA CCATCGCGTC GCAGCCCCTG | 60 |
| CD8α hinge | TCCCTGCGCC CAGAGGCGTG CCGGCCAGCG GCGGGGGGCG CAGTGCACAC GAGGGGGCTG | 120 |
| domain | GACTTCGCCT GTGAT | 135 |
| SEQ ID NO: 567 | ATTCCGTGGC TGGGCCATCT GCTGGTGGGC CTGAGCGGCG CGTTTGGCTT TATTATTCTG | 60 |
| IL-2Rβ | GTGTATCTGC TGATT | 75 |
| transmembrane | | |
| domain | | |
| SEQ ID NO: 568 | GAACCGAAAA GCTGCGATAA AACCCATACC TGCCCGCCGT GCCCGGCGCC GGAACTGCTG | 60 |
| IgG1 | GGCGGCCCGA GCGTGTTTCT GTTTCCGCCG AAA | 93 |
| transmembrane | | |
| and hinge | | |
| domain | | |
| SEQ ID NO: 569 | GAACCGAAAA GCTGCGATAA AACCCATACC TGCCCGCCGT GCCCGGCGCC GGAACTGCTG | 60 |
| IgG1 hinge | GGCGGCCCG | 69 |
| domain | | |
| SEQ ID NO: 570 | GAGAGCAAGT ACGGCCCTCC CTGCCCCCCT TGCCCTGCCC CCGAGTTCCT GGGCGGACCC | 60 |
| IgG4 hinge | AGCGTGTTCC TGTTCCCCCC CAAGCCCAAG GACACCCTGA TGATCAGCCG GACCCCCGAG | 120 |
| domain | GTGACCTGTG TGGTGGTGGA CGTGTCCCAG GAGGACCCCG AGGTCCAGTT CAACTGGTAC | 180 |
| | GTGGACGGCG TGGAGGTGCA CAACGCCAAG ACCAAGCCCC GGGAGGAGCA GTTCAATAGC | 240 |
| | ACCTACCGGG TGGTGTCCGT GCTGACCGTG CTGCACCAGG ACTGGCTGAA CGGCAAGGAA | 300 |
| | TACAAGTGTA AGGTGTCCAA CAAGGGCCTG CCCAGCAGCA TCGAGAAAAC CATCAGCAAG | 360 |
| | GCCAAGGGCC AGCCTCGGGA GCCCCAGGTG TACACCCTGC CCCCTAGCCA AGAGGAGATG | 420 |
| | ACCAAGAACC AGGTGTCCCT GACCTGCCTG GTGAAGGGCT TCTACCCCAG CGACATCGCC | 480 |
| | GTGGAGTGGG AGAGCAACGG CCAGCCCGAG AACAACTACA AGACCACCCC CCCTGTGCTG | 540 |
| | GACAGCGACG GCAGCTTCTT CCTGTACAGC CGGCTGACCG TGGACAAGAG CCGGTGGCAG | 600 |
| | GAGGGCAACG TCTTTAGCTG CTCCGTGATG CACGAGGCCC TGCACAACCA CTACACCCAG | 660 |
| | AAGAGCCTGA GCCTGTCCCT GGGCAAGATG | 690 |
| SEQ ID NO: 571 | AGGTGGCCCG AAAGTCCCAA GGCCCAGGCA TCTAGTGTTC CTACTGCACA GCCCCAGGCA | 60 |
| IgD hinge | GAAGGCAGCC TAGCCAAAGC TACTACTGCA CCTGCCACTA CGCGCAATAC TGGCCGTGGC | 120 |
| domain | GGGGAGGAGA AGAAAAAGGA GAAAGAGAAA GAAGAACAGG AAGAGAGGGA GACCAAGACC | 180 |
| | CCTGAATGTC CATCCCATAC CCAGCCGCTG GGCGTCTATC TCTTGACTCC CGCAGTACAG | 240 |
| | GACTTGTGGC TTAGAGATAA GGCCACCTTT ACATGTTTCG TCGTGGGCTC TGACCTGAAG | 300 |
| | GATGCCCATT TGACTTGGGA GGTTGCCGGA AAGGTACCCA CAGGGGGGGT TGAGGAAGGG | 360 |
| | TTGCTGGAGC GCCATTCCAA TGGCTCTCAG AGCCAGCACT CAAGACTCAC CCTTCCGAGA | 420 |
| | TCCCTGTGGA ACGCCGGGAC CTCTGTCACA TGTACTCTAA ATCATCCTAG CCTGCCCCCA | 480 |
| | CAGCGTCTGA TGGCCCTTAG AGAGCCAGCC GCCCAGGCAC CAGTTAAGCT TAGCCTGAAT | 540 |
| | CTGCTCGCCA GTAGTGATCC CCCAGAGGCC GCCAGCTGGC TCTTATGCGA AGTGTCCGGC | 600 |
| | TTTAGCCCGC CCAACATCTT GCTCATGTGG CTGGAGGACC AGCGAGAAGT GAACACCAGC | 660 |
| | GGCTTCGCTC CAGCCCGGCC CCCACCCCAG CCGGGTTCTA CCACATTCTG GGCCTGGAGT | 720 |
| | GTCTTAAGGG TCCCAGCACC ACCTAGCCCC CAGCCAGCCA CATACACCTG TGTTGTGTCC | 780 |
| | CATGAAGATA GCAGGACCCT GCTAAATGCT TCTAGGAGTC TGGAGGTTTC CTACGTGACT | 840 |
| | GACCATT | 847 |

In an embodiment, a transmembrane and/or hinge domain comprises a domain encoded by a nucleotide sequence, such domain selected from the group consisting of the alpha (α), beta (β), or zeta (ζ) chain of the T-cell receptor, CD3 epsilon (ε), CD4, CD5, CD8, CD9, CD16, CD22, CD27, CD28, CD33, CD37, CD45, CD64, CD80, CD86, CD134, CD137, CD154, IgG1, IgG4, IgD, IL-2Rα, IL-2Rβ, IL-2Rγ, and variants, fragments, and derivatives thereof. In an embodiment, a transmembrane and/or hinge domain is encoded by the sequence shown in SEQ ID NO:562. In an embodiment, a transmembrane and/or hinge domain comprises is encoded by a nucleotide that is at least 9900 identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571. In an embodiment, a transmembrane and/or hinge domain comprises is encoded by a nucleotide that is at least 98% identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571. In an embodiment, a transmembrane and/or hinge domain comprises is encoded by a nucleotide that is at least 97% identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571. In an embodiment, a transmembrane and/or hinge domain comprises is encoded by a nucleotide that is at least 96% identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571. In an embodiment, a transmembrane and/or hinge domain comprises is encoded by a nucleotide that is at least 95% identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571. In an embodiment, a transmembrane and/or hinge domain comprises is encoded by a nucleotide that is at least 90% identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571. In an embodiment, a transmembrane and/or hinge domain comprises is encoded by a nucleotide that is at least 85% identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571. In an embodiment, a transmembrane and/or hinge domain comprises is encoded by a nucleotide that is at least 80% identical to a sequence selected from the group consisting of SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, and SEQ ID NO:571.

In some embodiments, a CCR of the present invention includes an extracellular domain, a hinge domain, a transmembrane domain, and an intracellular domain, wherein the hinge domain is linked to the transmembrane domain by a linker comprising two to forty amino acids. In some embodiments, a CCR of the present invention includes an extracellular domain, a hinge domain, a transmembrane domain, and an intracellular domain, wherein the hinge domain is linked to the transmembrane domain by a linker selected from the group consisting of SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:74, SEQ ID NO:75, SEQ ID NO:76, SEQ ID NO:238, SEQ ID NO:239, SEQ ID NO:240, SEQ ID NO:241, SEQ ID NO:242, SEQ ID NO:243, SEQ ID NO:587, and fragments, variants, and derivatives thereof. Alternative linkers may also be used, as disclosed herein or known in the art, such as the linkers described in U.S. Pat. No. 9,394,368, the disclosure of which is incorporated by reference herein.

In some embodiments, a CCR of the present invention includes an extracellular domain, a hinge domain, a transmembrane domain, and an intracellular domain, wherein the extracellular domain comprises an scFv domain, and the hinge domain is linked to the scFv domain by a linker comprising two to forty amino acids. In some embodiments, a CCR of the present invention includes an extracellular domain, a hinge domain, a transmembrane domain, and an intracellular domain, wherein the extracellular domain comprises an scFv domain, and the hinge domain is linked to the scFv domain by a linker selected from the group consisting of SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:74, SEQ ID NO:75, SEQ ID NO:76, SEQ ID NO:238, SEQ ID NO:239, SEQ ID NO:240, SEQ ID NO:241, SEQ ID NO:242, SEQ ID NO:243, SEQ ID NO:587, and fragments, variants, and derivatives thereof. Alternative linkers may also be used, as disclosed herein or known in the art, such as the linkers described in U.S. Pat. No. 9,394,368, the disclosure of which is incorporated by reference herein.

In some embodiments, a CCR of the present invention includes an extracellular domain, a transmembrane domain, and an intracellular domain, wherein the extracellular domain comprises an scFv domain, and the transmembrane domain is linked to the scFv domain by a linker comprising two to forty amino acids. In some embodiments, a CCR of the present invention includes an extracellular domain, a transmembrane domain, and an intracellular domain, wherein the extracellular domain comprises an scFv domain, and the transmembrane domain is linked to the scFv domain by a linker selected from the group consisting of SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:74, SEQ ID NO:75, SEQ ID NO:76, SEQ ID NO:238, SEQ ID NO:239, SEQ ID NO:240, SEQ ID NO:241, SEQ ID NO:242, SEQ ID NO:243, SEQ ID NO:587, and fragments, variants, and derivatives thereof. Alternative linkers may also be used, as disclosed herein or known in the art, such as the linkers described in U.S. Pat. No. 9,394,368, the disclosure of which is incorporated by reference herein.

In an embodiment, a CCR of the present invention comprises a transmembrane domain that includes a CD40L (CD154) transmembrane domain. Suitable CD40L transmembrane domains are described in Aloui, et al., Int. J. Mol. Sci. 2014, 15(12), 22342-22364 and U.S. Pat. No. 10,287,354, the disclosures of each of which are incorporated by reference herein.

### C. Intracellular Domains

In an embodiment, a CCR comprises an intracellular domain, also referred to herein as a signaling domain, a costimulatory domain, or an endodomain. Such an intracellular domain may provide a costimulatory activation signal to a T cell or may activate alternative signaling pathways in a T cell useful in the present invention. In an embodiment, a CCR comprises an intracellular domain that is cytoplasmic. In an embodiment, a CCR comprises an intracellular domain that is cytoplasmic and transmembrane. In some embodiments, the intracellular domain is selected from the group consisting of alpha (α), beta (β), or zeta (ζ) chain of the T-cell receptor (including CD3ζ), CD3 epsilon (ε), CD4, CD5, CD8 (including CD8α), CD9, CD16, CD22, CD27, CD28, CD33, CD37, CD45, CD64, CD80, CD86, CD134 (OX40), CD137 (TNFRSF9, 4-1BB), CD150 (SLAM), CD270 (HVEM), CD278 (ICOS), CD357 (GITR), EphB6, STAT3, IL-2R, IL-2Rα, IL-2Rβ, IL-2Rγ, IL-7Rα, IL-12R1, IL-12R2, IL-15Rα, IL18-R1, IL-18RAP, IL-21R, and LTBR (lymphotoxin β receptor, TNFRSF3). In some embodiments, the full length of one of the foregoing protein sequences (except for the signal peptide) is employed in the intracellular domain. In some embodiments, a truncated portion or portions of one of the foregoing protein sequences is employed in the intracellular domain. In an embodiment, the intracellular domain is a full-length CD28 sequence. Suitable, non-limiting intracellular domains useful in the CCR constructs of the present invention are set forth in Table 58.

TABLE 58
Amino acid sequences of exemplary intracellular domains.

| Identifier | Sequence (One-Letter Amino Acid Symbols) | |
|---|---|---|
| SEQ ID NO: 572 | RSKRSRLLHS DYMNMTPRRP GPTRKHYQPY APPRDFAAYR S | 41 |
| CD28 | | |
| intracellular | | |
| domain | | |
| SEQ ID NO: 573 | ALYLLRRDQR LPPDAHKPPG GGSFRTPIQE EQADAHSTLA KI | 42 |
| CD134 (OX40) | | |
| intracellular | | |
| domain | | |
| SEQ ID NO: 574 | CWLTKKKYSS SVHDPNGEYM FMRAVNTAKK SRLTDVTL | 38 |
| CD278 (ICOS) | | |
| intracellular | | |
| domain | | |
| SEQ ID NO: 575 | KRGRKKLLYI FKQPFMRPVQ TTQEEDGCSC RFPEEEEGGC EL | 42 |
| CD137 (4-1BB) | | |
| intracellular | | |
| domain | | |
| SEQ ID NO: 576 | QRRKYRSNKG ESPVEPAEPC RYSCPREEEG STIPIQEDYR KPEPACSP | 48 |
| CD27 | | |
| intracellular | | |
| domain | | |
| SEQ ID NO: 577 | RVKFSRSADA PAYKQGQNQL YNELNLGRRE EYDVLDKRRG RDPEMGGKPR RKNPQEGLYN | 60 |
| CD3ζ | ELQKDKMAEA YSEIGMKGER RRGKGHDGLY QGLSTATKDT YDALHMQALP PR | 112 |
| intracellular | | |
| domain | | |
| SEQ ID NO: 578 | NCRNTGPWLK KVLKCNTPDP SKFFSQLSSE HGGDVQKWLS SPFPSSSFSP GGLAPEISPL | 60 |
| IL-2Rβ | EVLERDKVTQ LLLQQDKVPE PASLSSNHSL TSCFTNQGYF FFHLPDALEI EACQVYFTYD | 120 |
| intracellular | PYSEEDPDEG VAGAPTGSSP QPLQPLSGED DAYCTFPSRD DLLLFSPSLL GGPSPPSTAP | 180 |
| domain | GGSGAGEERM PPSLQERVPR DWDPQPLGPP TPGVPDLVDF QPPPELVLRE AGEEVPDAGP | 240 |
| | REGVSFPWSR PPGQGEFRAL NARLPLNTDA YLSLQELQGQ DPTHLV | 286 |
| SEQ ID NO: 579 | ERTMPRIPTL KNLEDLVTEY HGNFSAWSGV SKGLAESLQP DYSERLCLVS EIPPKGGALG | 60 |
| IL-2Rγ | EGPGASPCNQ HSPYWAPPCY TLKPET | 86 |
| intracellular | | |
| domain | | |
| SEQ ID NO: 580 | YRVDLVLFYR HLTRRDETLT DGKTYDAFVS YLKECRPENG EEHTFAVEIL PRVLEKHFGY | 60 |
| IL-18R1 | KLCIFERDVV PGGAVVDEIH SLIEKSRRLI IVLSKSYMSN EVRYELESGL HEALVERKIK | 120 |
| intracellular | IILIEFTPVT DFTFLPQSLK LLKSHRVLKW KADKSLSYNS RFWKNLLYLM PAKTVKPGRD | 180 |
| domain | EPEVLPVLSE SL | 192 |
| SEQ ID NO: 581 | KKRIKPIVWP SLPDHKKTLE HLCKKPRKNL NVSFNPESFL DCQIHRVDDI QARDEVEGFL | 60 |
| IL-7Rα | QDTFPQQLEE SEKQRLGGDV QSPNCPSEDV VITPESFGRD SSLTCLAGNV SACDAPILSS | 120 |
| intracellular | SRSLDCRESG KNGPHVYQDL LLSLGTTNST LPPPFSLQSG ILTLNPVAQG QPILTSLGSN | 180 |
| domain | QEEAYVTMSS FYQNQ | 195 |
| SEQ ID NO: 582 | NRAARHLCPP LPTPCASSAI EFPGGKETWQ WINPVDFQEE ASLQEALVVE MSWDKGERTE | 60 |
| IL-12R1 | PLEKTELPEG APELALDTEL SLEDGDRCKA KM | 92 |
| intracellular | | |
| domain | | |
| SEQ ID NO: 583 | HYFQQKVFVL LAALRPQWCS REIPDPANST CAKKYPIAEE KTQLPLDRLL IDWPTPEDPE | 60 |
| IL-12R2 | PLVISEVLHQ VTPVFRHPPC SNWPQREKGI QGHQASEKDM MHSASSPPPP RALQAESRQL | 120 |
| intracellular | VDLYKVLESR GSDPKPENPA CPWTVLPAGD LPTHDGYLPS NIDDLPSHEA PLADSLEELE | 180 |
| domain | PQHISLSVFP SSSLHPLTFS CGDKLTLDQL KMRCDSLML | 219 |
| SEQ ID NO: 584 | KSRQTPPLAS VEMEAMEALP VTWGTSSRDE DLENCSHHL | 39 |
| IL-15Rα | | |
| intracellular | | |
| domain | | |
| SEQ ID NO: 585 | SLKTHPLWRL WKKIWAVPSP ERFFMPLYKG CSGDFKKWVG APFTGSSLEL GPWSPEVPST | 60 |
| IL-21R | LEVYSCHPPR SPAKRLQLTE LQEPAELVES DGVPKPSFWP TAQNSGGSAY SEERDRPYGL | 120 |
| intracellular | VSIDTVTVLD AEGPCTWPCS CEDDGYPALD LDAGLEPSPG LEDPLLDAGT TVLSCGCVSA | 180 |
| domain | GSPGLGGPLG SLLDRLKPPL ADGEDWAGGL PWGGRSPGGV SESEAGSPLA GLDMDTFDSG | 240 |
| | FVGSDCSSPV ECDFTSPGDE GPPRSYLRQW VVIPPPLSSP GPQAS | 285 |
| SEQ ID NO: 586 | PLPPEMSGTM LMLAVLLPLA FFLLLATVFS CIWKSHPSLC RKLGSLLKRR PQGEGPNPVA | 60 |
| LTBR | GSWEPPKAHP YFPDLVQPLL PISGDVSPVS TGLPAAPVLE AGVPQQQSPL DLTREPQLEP | 120 |
| intracellular | GEQSQVAHGT NGIHVTGGSM TITGNIYIYN GPVLGGPPGP GDLPATPEPP YPIPEEGDPG | 180 |
| domain | PPGLSTPHQE DGKAWHLAET EHCGATPSNR GPRNQFITHD | 220 |
| SEQ ID NO: 587 | GGGGSGGGGS | 10 |
| linker | | |

In an embodiment, a CCR of the present invention includes a CD28 intracellular domain comprising the amino acid sequence of SEQ ID NO:572, or conservative amino acid substitutions thereof, or fragments, variants, or derivatives thereof, or an amino acid sequence that is at least 99% identical to the sequence given in SEQ ID NO:572, at least 98% identical to the sequence given in SEQ ID NO:572, at least 97% identical to the sequence given in SEQ ID NO:572, at least 96% identical to the sequence given in SEQ ID NO:572, at least 95% identical to the sequence given in SEQ ID NO:572, at least 90% identical to the sequence given in SEQ ID NO:572, at least 85% identical to the sequence given in SEQ ID NO:572, or at least 80% identical to the sequence given in SEQ ID NO:572.

In an embodiment, a CCR of the present invention includes a CD134 (OX40) intracellular domain comprising the amino acid sequence of SEQ ID NO:573, or conservative amino acid substitutions thereof, or fragments, variants, or derivatives thereof, or an amino acid sequence that is at least 99% identical to the sequence given in SEQ ID NO:573, at least 98% identical to the sequence given in SEQ ID NO:573, at least 97% identical to the sequence given in SEQ ID NO:573, at least 96% identical to the sequence given in SEQ ID NO:573, at least 95% identical to the sequence given in SEQ ID NO:573, at least 90% identical to the sequence given in SEQ ID NO:573, at least 85% identical to the sequence given in SEQ ID NO:573, or at least 80% identical to the sequence given in SEQ ID NO:573.

In an embodiment, a CCR of the present invention includes a CD278 (ICOS) intracellular domain comprising the amino acid sequence of SEQ ID NO:574, or conservative amino acid substitutions thereof, or fragments, variants, or derivatives thereof, or an amino acid sequence that is at least 99% identical to the sequence given in SEQ ID NO:574, at least 98% identical to the sequence given in SEQ ID NO:574, at least 97% identical to the sequence given in SEQ ID NO:574, at least 96% identical to the sequence given in SEQ ID NO:574, at least 95% identical to the sequence given in SEQ ID NO:574, at least 90% identical to the sequence given in SEQ ID NO:574, at least 85% identical to the sequence given in SEQ ID NO:574, or at least 80% identical to the sequence given in SEQ ID NO:574.

In an embodiment, a CCR of the present invention includes a CD137 (4-1BB) intracellular domain comprising the amino acid sequence of SEQ ID NO:575, or conservative amino acid substitutions thereof, or fragments, variants, or derivatives thereof, or an amino acid sequence that is at least 99% identical to the sequence given in SEQ ID NO:575, at least 98% identical to the sequence given in SEQ ID NO:575, at least 97% identical to the sequence given in SEQ ID NO:575, at least 96% identical to the sequence given in SEQ ID NO:575, at least 95% identical to the sequence given in SEQ ID NO:575, at least 90% identical to the sequence given in SEQ ID NO:575, at least 85% identical to the sequence given in SEQ ID NO:575, or at least 80% identical to the sequence given in SEQ ID NO:575.

In an embodiment, a CCR of the present invention includes a CD27 intracellular domain comprising the amino acid sequence of SEQ ID NO:576, or conservative amino acid substitutions thereof, or fragments, variants, or derivatives thereof, or an amino acid sequence that is at least 99% identical to the sequence given in SEQ ID NO:576, at least 98% identical to the sequence given in SEQ ID NO:576, at least 97% identical to the sequence given in SEQ ID NO:576, at least 96% identical to the sequence given in SEQ ID NO:576, at least 95% identical to the sequence given in SEQ ID NO:576, at least 90% identical to the sequence given in SEQ ID NO:576, at least 85% identical to the sequence given in SEQ ID NO:576, or at least 80% identical to the sequence given in SEQ ID NO:576.

In an embodiment, a CCR of the present invention includes a CD3ζ intracellular domain comprising the amino acid sequence of SEQ ID NO:577, or conservative amino acid substitutions thereof, or fragments, variants, or derivatives thereof, or an amino acid sequence that is at least 99% identical to the sequence given in SEQ ID NO:577, at least 98% identical to the sequence given in SEQ ID NO:577, at least 97% identical to the sequence given in SEQ ID NO:577, at least 96% identical to the sequence given in SEQ ID NO:577, at least 95% identical to the sequence given in SEQ ID NO:577, at least 90% identical to the sequence given in SEQ ID NO:577, at least 85% identical to the sequence given in SEQ ID NO:577, or at least 80% identical to the sequence given in SEQ ID NO:577.

In an embodiment, a CCR of the present invention includes a IL-2Rβ intracellular domain comprising the amino acid sequence of SEQ ID NO:578, or conservative amino acid substitutions thereof, or fragments, variants, or derivatives thereof, or an amino acid sequence that is at least 99% identical to the sequence given in SEQ ID NO:578, at least 98% identical to the sequence given in SEQ ID NO:578, at least 97% identical to the sequence given in SEQ ID NO:578, at least 96% identical to the sequence given in SEQ ID NO:578, at least 95% identical to the sequence given in SEQ ID NO:578, at least 90% identical to the sequence given in SEQ ID NO:578, at least 85% identical to the sequence given in SEQ ID NO:578, or at least 80% identical to the sequence given in SEQ ID NO:578.

In an embodiment, a CCR of the present invention includes a IL-2Rγ intracellular domain comprising the amino acid sequence of SEQ ID NO:579, or conservative amino acid substitutions thereof, or fragments, variants, or derivatives thereof, or an amino acid sequence that is at least 99% identical to the sequence given in SEQ ID NO:579, at least 98% identical to the sequence given in SEQ ID NO:579, at least 97% identical to the sequence given in SEQ ID NO:579, at least 96% identical to the sequence given in SEQ ID NO:579, at least 95% identical to the sequence given in SEQ ID NO:579, at least 90% identical to the sequence given in SEQ ID NO:579, at least 85% identical to the sequence given in SEQ ID NO:579, or at least 80% identical to the sequence given in SEQ ID NO:579.

In an embodiment, a CCR of the present invention includes a IL-18R1 intracellular domain comprising the amino acid sequence of SEQ ID NO:580, or conservative amino acid substitutions thereof, or fragments, variants, or derivatives thereof, or an amino acid sequence that is at least 99% identical to the sequence given in SEQ ID NO:580, at least 98% identical to the sequence given in SEQ ID NO:580, at least 97% identical to the sequence given in SEQ ID NO:580, at least 96% identical to the sequence given in SEQ ID NO:580, at least 95% identical to the sequence given in SEQ ID NO:580, at least 90% identical to the sequence given in SEQ ID NO:580, at least 85% identical to the sequence given in SEQ ID NO:580, or at least 80% identical to the sequence given in SEQ ID NO:580.

In an embodiment, a CCR of the present invention includes a IL-7Rα intracellular domain comprising the amino acid sequence of SEQ ID NO:581, or conservative amino acid substitutions thereof, or fragments, variants, or derivatives thereof, or an amino acid sequence that is at least 99% identical to the sequence given in SEQ ID NO:581, at least 98% identical to the sequence given in SEQ ID NO:581, at least 97% identical to the sequence given in SEQ ID NO:581, at least 96% identical to the sequence given in SEQ ID NO:581, at least 95% identical to the sequence given in SEQ ID NO:581, at least 90% identical to the sequence given in SEQ ID NO:581, at least 85% identical to the sequence given in SEQ ID NO:581, or at least 80% identical to the sequence given in SEQ ID NO:581.

In an embodiment, a CCR of the present invention includes a IL-12R1 intracellular domain comprising the amino acid sequence of SEQ ID NO:582, or conservative amino acid substitutions thereof, or fragments, variants, or derivatives thereof, or an amino acid sequence that is at least 99% identical to the sequence given in SEQ ID NO:582, at least 98% identical to the sequence given in SEQ ID NO:582, at least 97% identical to the sequence given in SEQ ID NO:582, at least 96% identical to the sequence given in SEQ ID NO:582, at least 95% identical to the sequence given in SEQ ID NO:582, at least 90% identical to the sequence given in SEQ ID NO:582, at least 85% identical to the sequence given in SEQ ID NO:582, or at least 80% identical to the sequence given in SEQ ID NO:582.

In an embodiment, a CCR of the present invention includes a IL-12R2 intracellular domain comprising the amino acid sequence of SEQ ID NO:583, or conservative amino acid substitutions thereof, or fragments, variants, or derivatives thereof, or an amino acid sequence that is at least 99% identical to the sequence given in SEQ ID NO:583, at least 98% identical to the sequence given in SEQ ID NO:583, at least 97% identical to the sequence given in SEQ ID NO:583, at least 96% identical to the sequence given in SEQ ID NO:583, at least 95% identical to the sequence given in SEQ ID NO:583, at least 90% identical to the sequence given in SEQ ID NO:583, at least 85% identical to the sequence given in SEQ ID NO:583, or at least 80% identical to the sequence given in SEQ ID NO:583.

In an embodiment, a CCR of the present invention includes a IL-15Rα intracellular domain comprising the amino acid sequence of SEQ ID NO:584, or conservative amino acid substitutions thereof, or fragments, variants, or derivatives thereof, or an amino acid sequence that is at least 99% identical to the sequence given in SEQ ID NO:584, at least 98% identical to the sequence given in SEQ ID NO:584, at least 97% identical to the sequence given in SEQ ID NO:584, at least 96% identical to the sequence given in SEQ ID NO:584, at least 95% identical to the sequence given in SEQ ID NO:584, at least 90% identical to the sequence given in SEQ ID NO:584, at least 85% identical to the sequence given in SEQ ID NO:584, or at least 80% identical to the sequence given in SEQ ID NO:584.

In an embodiment, a CCR of the present invention includes a IL-21R intracellular domain comprising the amino acid sequence of SEQ ID NO:585, or conservative amino acid substitutions thereof, or fragments, variants, or derivatives thereof, or an amino acid sequence that is at least 99% identical to the sequence given in SEQ ID NO:585, at least 98% identical to the sequence given in SEQ ID NO:585, at least 97% identical to the sequence given in SEQ ID NO:585, at least 96% identical to the sequence given in SEQ ID NO:585, at least 95% identical to the sequence given in SEQ ID NO:585, at least 90% identical to the sequence given in SEQ ID NO:585, at least 85% identical to the sequence given in SEQ ID NO:585, or at least 80% identical to the sequence given in SEQ ID NO:585.

In an embodiment, a CCR of the present invention includes a LTBR intracellular domain comprising the amino acid sequence of SEQ ID NO:586, or conservative amino acid substitutions thereof, or fragments, variants, or derivatives thereof, or an amino acid sequence that is at least 99% identical to the sequence given in SEQ ID NO:586, at least 98% identical to the sequence given in SEQ ID NO:586, at least 97% identical to the sequence given in SEQ ID NO:586, at least 96% identical to the sequence given in SEQ ID NO:586, at least 95% identical to the sequence given in SEQ ID NO:586, at least 90% identical to the sequence given in SEQ ID NO:586, at least 85% identical to the sequence given in SEQ ID NO:586, or at least 80% identical to the sequence given in SEQ ID NO:586.

In some embodiments, the intracellular domain may be linked directly to the transmembrane domain. In some embodiments, the intracellular domain may be linked to the transmembrane domain through a linker. Optionally, in some embodiments, a short oligo- or polypeptide linker, between 2 and 10 amino acids in length, may form the linkage between the transmembrane domain and the intracellular domain of the CCR, and in some embodiments, this linker may comprise a glycine-serine doublet, or other alternative linkers, as described elsewhere herein. In some embodiments, the linker comprises an amino acid sequence selected from the group consisting of SEQ ID NO:587, SEQ ID NO:238, SEQ ID NO:239, SEQ ID NO:240, SEQ ID NO:241, SEQ ID NO:242, SEQ ID NO:243, SEQ ID NO:63, SEQ ID NO:64, SEQ ID NO:65, SEQ ID NO:66, SEQ ID NO:67, SEQ ID NO:68, SEQ ID NO:69, SEQ ID NO:70. SEQ ID NO:71, SEQ ID NO:72, SEQ ID NO:74, SEQ ID NO:75, and SEQ ID NO:76.

The nucleotide sequences encoding exemplary intracellular domains for use with CCRs of the present invention are provided in Table 59. In an embodiment, a nucleotide sequence in Table 59 is codon-optimized to improve protein expression.

TABLE 59
Nucleotide sequences of exemplary intracellular signaling domains.

| Identifier | Sequence (One-Letter Nucleotide Symbols) | |
|---|---|---|
| SEQ ID NO: 588 | AGGAGTAAGA GGAGCAGGCT CCTGCACAGT GACTACATGA ACATGACTCC CCGCCGCCCC | 60 |
| CD28 | GGGCCCACCC GCAAGCATTA CCAGCCCTAT GCCCCACCAC GCGACTTCGC AGCCTATCGC | 120 |
| intracellular | TCC | 123 |
| domain | | |
| SEQ ID NO: 589 | GCGCTGTATC TGCTGCGCCG CGATCAGCGC CTGCCGCCGG ATGCGCATAA ACCGCCGGGC | 60 |
| CD134 (OX40) | GGCGGCAGCT TTCGCACCCC GATTCAGGAA GAACAGGCGG ATGCGCATAG CACCCTGGCG | 120 |
| intracellular | AAAATT | 126 |
| domain | | |
| SEQ ID NO: 590 | ACAAAAAAGA AGTATTCATC CAGTGTGCAC GACCCTAACG GTGAATACAT GTTCATGAGA | 60 |
| CD278 (ICOS) | GCAGTGAACA CAGCCAAAAA ATCCAGACTC ACAGATGTGA CCCTA | 105 |
| intracellular | | |
| domain | | |
| SEQ ID NO: 591 | AAACGGGGCA GAAAGAAACT CCTGTATATA TTCAAACAAC CATTTATGAG ACCAGTACAA | 60 |
| CD137 (4-1BB) | ACTACTCAAG AGGAAGATGG CTGTAGCTGC CGATTTCCAG AAGAAGAAGA AGGAGGATGT | 120 |
| intracellular | GAACTG | 126 |
| domain | | |
| SEQ ID NO: 592 | CAACGAAGGA AATATAGATC AAACAAAGGA GAAAGTCCTG TGGAGCCTGC AGAGCCTTGT | 60 |
| CD27 | CGTTACAGCT GCCCCAGGGA GGAGGAGGGC AGCACCATCC CCATCCAGGA GGATTACCGA | 120 |
| intracellular | AAACCGGAGC CTGCCTGCTC CCCC | 144 |
| domain | | |
| SEQ ID NO: 593 | AGAGTGAAGT TCAGCAGGAG CGCAGACGCC CCCGCGTACA AGCAGGGCCA GAACCAGCTC | 60 |
| CD3ζ | TATAACGAGC TCAATCTAGG ACGAAGAGAG GAGTACGATG TTTTGGACAA GAGACGTGGC | 120 |
| intracellular | CGGGACCCTG AGATGGGGGG AAAGCCGAGA AGGAAGAACC CTCAGGAAGG CCTGTACAAT | 180 |
| domain | GAACTGCAGA AAGATAAGAT GGCGGAGGCC TACAGTGAGA TTGGGATGAA AGGCGAGCGC | 240 |
| | CGGAGGGGCA AGGGGCACGA TGGCCTTTAC CAGGGTCTCA GTACAGCCAC CAAGGACACC | 300 |
| | TACGACGCCC TTCACATGCA GGCCCTGCCC CCTCGC | 336 |
| SEQ ID NO: 594 | AACTGCAGGA ACACCGGGCC ATGGCTGAAG AAGGTCCTGA AGTGTAACAC CCCAGACCCC | 60 |
| IL-2Rβ | TCGAAGTTCT TTTCCCAGCT GAGCTCAGAG CATGGAGGAG ACGTCCAGAA GTGGCTCTCT | 120 |
| intracellular | TCGCCCTTCC CCTCATCGTC CTTCAGCCCT GGCGGCCTGG CACCTGAGAT CTCGCCACTA | 180 |
| domain | GAAGTGCTGG AGAGGGACAA GGTGACGCAG CTGCTCCTGC AGCAGGACAA GGTGCCTGAG | 240 |
| | CCCGCATCCT TAAGCAGCAA CCACTCGCTG ACCAGCTGCT TCACCAACCA GGGTTACTTC | 300 |
| | TTCTTCCACC TCCCGGATGC CTTGGAGATA GAGGCCTGCC AGGTGTACTT TACTTACGAC | 360 |
| | CCCTACTCAG AGGAAGACCC TGATGAGGGT GTGGCCGGGG CACCCACAGG GTCTTCCCCC | 420 |
| | CAACCCCTGC AGCCTCTGTC AGGGGAGGAC GACGCCTACT GCACCTTCCC CTCCAGGGAT | 480 |
| | GACCTGCTGC TCTTCTCCCC CAGTCTCCTC GGTGGCCCCA GCCCCCCAAG CACTGCCCCT | 540 |
| | GGGGGCAGTG GGGCCGGTGA AGAGAGGATG CCCCCTTCTT TGCAAGAAAG AGTCCCCAGA | 600 |
| | GACTGGGACC CCCAGCCCCT GGGGCCTCCC ACCCCAGGAG TCCCAGACCT GGTGGATTTT | 660 |
| | CAGCCACCCC CTGAGCTGGT GCTGCGAGAG GCTGGGGAGG AGGTCCCTGA CGCTGGCCCC | 720 |
| | AGGGAGGGAG TCAGTTTCCC CTGGTCCAGG CCTCCTGGGC AGGGGGAGTT CAGGGCCCTT | 780 |
| | AATGCTCGCC TGCCCCTGAA CACTGATGCC TACTTGTCCC TCCAAGAACT CCAGGGTCAG | 840 |
| | GACCCAACTC ACTTGGTG | 858 |
| SEQ ID NO: 595 | ATGGGAATGA AGACACCACA GCTGGAACAA TCAGTGGATT ATAGACATAA GTTCTCCTTG | 60 |
| IL-2Rγ | CCTAGTGTGG ATGGGCAGAA ACGCTACACG TTTCGTGTTC GGAGCCGCTT TAACCCACTC | 120 |
| intracellular | TGTGGAAGTG CTCAGCATTG GAGTGAATGG AGCCACCCAA TCCACTGGGG GAGCAATACT | 180 |
| domain | TCAAAAGAGA ATCCTTTCCT GTTTGCATTG GAAGCCGTGG TTATCTCTGT TGGCTCCATG | 240 |
| | GGATTGATTA TCAGCCTTCT CTGTGTGTAT TTCTGGCTGG AACGGACGAT GCCCCGAATT | 300 |
| | CCCACCCTGA AGAACCTAGA GGATCTTGTT ACTGAATACC ACGGGAACTT TTCGGCCTGG | 360 |
| | AGTGGTGTGT CTAAGGGACT GGCTGAGAGT CTGCAGCCAG ACTACAGTGA ACGACTCTGC | 420 |
| | CTCGTCAGTG AGATTCCCCC AAAAGGAGGG GCCCTTGGGG AGGGGCCTGG GGCCTCCCCA | 480 |
| | TGCAACCAGC ATAGCCCCTA CTGGGCCCCC CCATGTTACA CCCTAAAGCC TGAAACCTGA | 540 |
| SEQ ID NO: 596 | TATAGAGTTG ACTTGGTTCT ATTTTATAGA CATTTAACGA GAAGAGATGA AACATTAACA | 60 |
| IL-18R1 | GATGGAAAAA CATATGATGC TTTTGTGTCT TACCTAAAAG AATGCCGACC TGAAAATGGA | 120 |
| intracellular | GAGGAGCACA CCTTTGCTGT GGAGATTTTG CCCAGGGTGT TGGAGAAACA TTTTGGGTAT | 180 |
| domain | AAGTTATGCA TATTTGAAAG GGATGTAGTG CCTGGAGGAG CTGTTGTTGA TGAAATCCAC | 240 |
| | TCACTGATAG AGAAAAGCCG AAGACTAATC ATTGTCCTAA GTAAAAGTTA TATGTCTAAT | 300 |
| | GAGGTCAGGT ATGAACTTGA AAGTGGACTC CATGAAGCAT TGGTGGAAAG AAAAATTAAA | 360 |
| | ATAATCTTAA TTGAATTTAC ACCTGTTACT GACTTCACAT TCTTGCCCCA ATCACTAAAG | 420 |
| | CTTTTGAAAT CTCACAGAGT TCTGAAGTGG AAGGCCGATA AATCTCTTTC TTATAACTCA | 480 |
| | AGGTTCTGGA AGAACCTTCT TTACTTAATG CCTGCAAAAA CAGTCAAGCC AGGTAGAGAC | 540 |
| | GAACCGGAAG TCTTGCCTGT TCTTTCCGAG TCT | 573 |
| SEQ ID NO: 597 | AAAAAACGCA TTAAACCGAT TGTGTGGCCG AGCCTGCCGG ATCATAAAAA AACCCTGGAA | 60 |
| IL-7Rα | CATCTGTGCA AAAAACCGCG CAAAAACCTG AACGTGAGCT TTAACCCGGA AAGCTTTCTG | 120 |
| intracellular | GATTGCCAGA TTCATCGCGT GGATGATATT CAGGCGCGCG ATGAAGTGGA AGGCTTTCTG | 180 |
| domain | CAGGATACCT TTCCGCAGCA GCTGGAAGAA AGCGAAAAAC AGCGCCTGGG CGGCGATGTG | 240 |
| | CAGAGCCCGA ACTGCCCGAG CGAAGATGTG GTGATTACCC CGGAAAGCTT TGGCCGCGAT | 300 |
| | AGCAGCCTGA CCTGCCTGGC GGGCAACGTG AGCGCGTGCG ATGCGCCGAT TCTGAGCAGC | 360 |
| | AGCCGCAGCC TGGATTGCCG CGAAAGCGGC AAAAACGGCC CGCATGTGTA TCAGGATCTG | 420 |
| | CTGCTGAGCC TGGGCACCAC CAACAGCACC CTGCCGCCGC CGTTTAGCCT GCAGAGCGGC | 480 |
| | ATTCTGACCC TGAACCCGGT GGCGCAGGGC CAGCCGATTC TGACCAGCCT GGGCAGCAAC | 540 |
| | CAGGAAGAAG CGTATGTGAC CATGAGCAGC TTTTATCAGA ACCAG | 585 |
| SEQ ID NO: 598 | AACCGCGCGG CGCGCCATCT GTGCCCGCCG CTGCCGACCC CGTGCGCGAG CAGCGCGATT | 60 |
| IL-12R1 | GAATTTCCGG GCGGCAAAGA AACCTGGCAG TGGATTAACC CGGTGGATTT TCAGGAAGAA | 120 |
| intracellular | GCGAGCCTGC AGGAAGCGCT GGTGGTGGAA ATGAGCTGGG ATAAAGGCGA ACGCACCGAA | 180 |
| domain | CCGCTGGAAA AAACCGAACT GCCGGAAGGC GCGCCGGAAC TGGCGCTGGA TACCGAACTG | 240 |
| | AGCCTGGAAG ATGGCGATCG CTGCAAAGCG AAAATG | 276 |
| SEQ ID NO: 599 | CATTATTTTC AGCAGAAAGT GTTTGTGCTG CTGGCGGCGC TGCGCCCGCA GTGGTGCAGC | 60 |
| IL-12R2 | CGCGAAATTC CGGATCCGGC GAACAGCACC TGCGCGAAAA AATATCCGAT TGCGGAAGAA | 120 |
| intracellular | AAAACCCAGC TGCCGCTGGA TCGCCTGCTG ATTGATTGGC CGACCCCGGA AGATCCGGAA | 180 |
| domain | CCGCTGGTGA TTAGCGAAGT GCTGCATCAG GTGACCCCGG TGTTTCGCCA TCCGCCGTGC | 240 |
| | AGCAACTGGC CGCAGCGCGA AAAAGGCATT CAGGGCCATC AGGCGAGCGA AAAAGATATG | 300 |
| | ATGCATAGCG CGAGCAGCCC GCCGCCGCCG CGCGCGCTGC AGGCGGAAAG CCGCCAGCTG | 360 |
| | GTGGATCTGT ATAAAGTGCT GGAAAGCCGC GGCAGCGATC CGAAACCGGA AAACCCGGCG | 420 |
| | TGCCCGTGGA CCGTGCTGCC GGCGGGCGAT CTGCCGACCC ATGATGGCTA TCTGCCGAGC | 480 |
| | AACATTGATG ATCTGCCGAG CCATGAAGCG CCGCTGGCGG ATAGCCTGGA AGAACTGGAA | 540 |
| | CCGCAGCATA TTAGCCTGAG CGTGTTTCCG AGCAGCAGCC TGCATCCGCT GACCTTTAGC | 600 |
| | TGCGGCGATA AACTGACCCT GGATCAGCTG AAAATGCGCT GCGATAGCCT GATGCTG | 657 |
| SEQ ID NO: 600 | AAAAGCCGCC AGACCCCGCC GCTGGCGAGC GTGGAAATGG AAGCGATGGA AGCGCTGCCG | 60 |
| IL-15Rα | GTGACCTGGG GCACCAGCAG CCGCGATGAA GATCTGGAAA ACTGCAGCCA TCATCTG | 117 |
| intracellular | | |
| domain | | |
| SEQ ID NO: 601 | AGCCTGAAAA CCCATCCGCT GTGGCGCCTG TGGAAAAAAA TTTGGGCGGT GCCGAGCCCG | 60 |
| IL-21R | GAACGCTTTT TTATGCCGCT GTATAAAGGC TGCAGCGGCG ATTTTAAAAA ATGGGTGGGC | 120 |
| intracellular | GCGCCGTTTA CCGGCAGCAG CCTGGAACTG GGCCCGTGGA GCCCGGAAGT GCCGAGCACC | 180 |
| domain | CTGGAAGTGT ATAGCTGCCA TCCGCCGCGC AGCCCGGCGA AACGCCTGCA GCTGACCGAA | 240 |
| | CTGCAGGAAC CGGCGGAACT GGTGGAAAGC GATGGCGTGC CGAAACCGAG CTTTTGGCCG | 300 |
| | ACCGCGCAGA ACAGCGGCGG CAGCGCGTAT AGCGAAGAAC GCGATCGCCC GTATGGCCTG | 360 |
| | GTGAGCATTG ATACCGTGAC CGTGCTGGAT GCGGAAGGCC CGTGCACCTG GCCGTGCAGC | 420 |
| | TGCGAAGATG ATGGCTATCC GGCGCTGGAT CTGGATGCGG GCCTGGAACC GAGCCCGGGC | 480 |
| | CTGGAAGATC CGCTGCTGGA TGCGGGCACC ACCGTGCTGA GCTGCGGCTG CGTGAGCGCG | 540 |
| | GGCAGCCCGG GCCTGGGCGG CCCGCTGGGC AGCCTGCTGG ATCGCCTGAA ACCGCCGCTG | 600 |
| | GCGGATGGCG AAGATTGGGC GGGCGGCCTG CCGTGGGGCG GCCGCAGCCC GGGCGGCGTG | 660 |
| | AGCGAAAGCG AAGCGGGCAG CCCGCTGGCG GGCCTGGATA TGGATACCTT TGATAGCGGC | 720 |
| | TTTGTGGGCA GCGATTGCAG CAGCCCGGTG GAATGCGATT TTACCAGCCC GGGCGATGAA | 780 |
| | GGCCCGCCGC GCAGCTATCT GCGCCAGTGG GTGGTGATTC CGCCGCCGCT GAGCAGCCCG | 840 |
| | GGCCCGCAGG CGAGC | 855 |
| SEQ ID NO: 602 | CCGCTGCCGC CGGAAATGAG CGGCACCATG CTGATGCTGG CGGTGCTGCT GCCGCTGGCG | 60 |
| LTBR | TTTTTTCTGC TGCTGGCGAC CGTGTTTAGC TGCATTTGGA AAAGCCATCC GAGCCTGTGC | 120 |
| intracellular | CGCAAACTGG GCAGCCTGCT GAAACGCCGC CCGCAGGGCG AAGGCCCGAA CCCGGTGGCG | 180 |
| domain | GGCAGCTGGG AACCGCCGAA AGCGCATCCG TATTTTCCGG ATCTGGTGCA GCCGCTGCTG | 240 |
| | CCGATTAGCG GCGATGTGAG CCCGGTGAGC ACCGGCCTGC CGGCGGCGCC GGTGCTGGAA | 300 |
| | GCGGGCGTGC CGCAGCAGCA GAGCCCGCTG GATCTGACCC GCGAACCGCA GCTGGAACCG | 360 |
| | GGCGAACAGA GCCAGGTGGC GCATGGCACC AACGGCATTC ATGTGACCGG CGGCAGCATG | 420 |
| | ACCATTACCG GCAACATTTA TATTTATAAC GGCCCGGTGC TGGGCGGCCC GCCGGGCCCG | 480 |
| | GGCGATCTGC CGGCGACCCC GGAACCGCCG TATCCGATTC CGGAAGAAGG CGATCCGGGC | 540 |
| | CCGCCGGGCC TGAGCACCCC GCATCAGGAA GATGGCAAAG CGTGGCATCT GGCGGAAACC | 600 |
| | GAACATTGCG GCGCGACCCC GAGCAACCGC GGCCCGCGCA ACCAGTTTAT TACCCATGAT | 660 |
| SEQ ID NO: 603 | GGTGGCGGAG GTTCTGGAGG TGGAGGTTCC | 30 |
| linker | | |

In an embodiment, a CCR of the present invention includes a CD28 intracellular domain encoded by a nucleotide comprising the sequence of SEQ ID NO:588, or a sequence that is at least 99% identical to the sequence given in SEQ ID NO:588, at least 98% identical to the sequence given in SEQ ID NO:588, at least 97% identical to the sequence given in SEQ ID NO:588, at least 96% identical to the sequence given in SEQ ID NO:588, at least 95% identical to the sequence given in SEQ ID NO:588, at least 90% identical to the sequence given in SEQ ID NO:588, at least 85% identical to the sequence given in SEQ ID NO:588, or at least 80% identical to the sequence given in SEQ ID NO:588.

In an embodiment, a CCR of the present invention includes a CD134 intracellular domain, also referred to as an OX40 intracellular domain, encoded by a nucleotide comprising the sequence of SEQ ID NO:589, or a sequence that is at least 99% identical to the sequence given in SEQ ID NO:589, at least 98% identical to the sequence given in SEQ ID NO:589, at least 97% identical to the sequence given in SEQ ID NO:589, at least 96% identical to the sequence given in SEQ ID NO:589, at least 95% identical to the sequence given in SEQ ID NO:589, at least 90% identical to the sequence given in SEQ ID NO:589, at least 85% identical to the sequence given in SEQ ID NO:589, or at least 80% identical to the sequence given in SEQ ID NO:589.

In an embodiment, a CCR of the present invention includes a CD278 intracellular domain, also referred to as an ICOS intracellular domain, encoded by a nucleotide comprising the sequence of SEQ ID NO:590, or a sequence that is at least 99% identical to the sequence given in SEQ ID NO:590, at least 98% identical to the sequence given in SEQ ID NO:590, at least 97% identical to the sequence given in SEQ ID NO:590, at least 96% identical to the sequence given in SEQ ID NO:590, at least 95% identical to the sequence given in SEQ ID NO:590, at least 90% identical to the sequence given in SEQ ID NO:590, at least 85% identical to the sequence given in SEQ ID NO:590, or at least 80% identical to the sequence given in SEQ ID NO:590.

In an embodiment, a CCR of the present invention includes a CD137 intracellular domain, also referred to as a 4-1BB intracellular domain, encoded by a nucleotide comprising the sequence of SEQ ID NO:591, or a sequence that is at least 99% identical to the sequence given in SEQ ID NO:591, at least 98% identical to the sequence given in SEQ ID NO:591, at least 97% identical to the sequence given in SEQ ID NO:591, at least 96% identical to the sequence given in SEQ ID NO:591, at least 95% identical to the sequence given in SEQ ID NO:591, at least 90% identical to the sequence given in SEQ ID NO:591, at least 85% identical to the sequence given in SEQ ID NO:591, or at least 80% identical to the sequence given in SEQ ID NO:591.

In an embodiment, a CCR of the present invention includes a CD27 intracellular domain, encoded by a nucleotide comprising the sequence of SEQ ID NO:592, or a sequence that is at least 99% identical to the sequence given in SEQ ID NO:592, at least 98% identical to the sequence given in SEQ ID NO:592, at least 97% identical to the sequence given in SEQ ID NO:592, at least 96% identical to the sequence given in SEQ ID NO:592, at least 95% identical to the sequence given in SEQ ID NO:592, at least 90% identical to the sequence given in SEQ ID NO:592, at least 85% identical to the sequence given in SEQ ID NO:592, or at least 80% identical to the sequence given in SEQ ID NO:592.

In an embodiment, a CCR of the present invention includes a CD3ζ intracellular domain, encoded by a nucleotide comprising the sequence of SEQ ID NO:593, or a sequence that is at least 99% identical to the sequence given in SEQ ID NO:593, at least 98% identical to the sequence given in SEQ ID NO:593, at least 97% identical to the sequence given in SEQ ID NO:593, at least 96% identical to the sequence given in SEQ ID NO:593, at least 95% identical to the sequence given in SEQ ID NO:593, at least 90% identical to the sequence given in SEQ ID NO:593, at least 85% identical to the sequence given in SEQ ID NO:593, or at least 80% identical to the sequence given in SEQ ID NO:593.

In an embodiment, a CCR of the present invention includes an IL-2Rβ intracellular domain, encoded by a nucleotide comprising the sequence of SEQ ID NO:594, or a sequence that is at least 99% identical to the sequence given in SEQ ID NO:594, at least 98% identical to the sequence given in SEQ ID NO:594, at least 97% identical to the sequence given in SEQ ID NO:594, at least 96% identical to the sequence given in SEQ ID NO:594, at least 95% identical to the sequence given in SEQ ID NO:594, at least 90% identical to the sequence given in SEQ ID NO:594, at least 85% identical to the sequence given in SEQ ID NO:594, or at least 80% identical to the sequence given in SEQ ID NO:594.

In an embodiment, a CCR of the present invention includes an IL-2Rγ intracellular domain, encoded by a nucleotide comprising the sequence of SEQ ID NO:595, or a sequence that is at least 99% identical to the sequence given in SEQ ID NO:595, at least 98% identical to the sequence given in SEQ ID NO:595, at least 97% identical to the sequence given in SEQ ID NO:595, at least 96% identical to the sequence given in SEQ ID NO:595, at least 95% identical to the sequence given in SEQ ID NO:595, at least 90% identical to the sequence given in SEQ ID NO:595, at least 85% identical to the sequence given in SEQ ID NO:595, or at least 80% identical to the sequence given in SEQ ID NO:595.

In an embodiment, a CCR of the present invention includes an IL-18R1 intracellular domain, encoded by a nucleotide comprising the sequence of SEQ ID NO:596, or a sequence that is at least 99% identical to the sequence given in SEQ ID NO:596, at least 98% identical to the sequence given in SEQ ID NO:596, at least 97% identical to the sequence given in SEQ ID NO:596, at least 96% identical to the sequence given in SEQ ID NO:596, at least 95% identical to the sequence given in SEQ ID NO:596, at least 90% identical to the sequence given in SEQ ID NO:596, at least 85% identical to the sequence given in SEQ ID NO:596, or at least 80% identical to the sequence given in SEQ ID NO:596.

In an embodiment, a CCR of the present invention includes an IL-7Rα intracellular domain, encoded by a nucleotide comprising the sequence of SEQ ID NO:597, or a sequence that is at least 99% identical to the sequence given in SEQ ID NO:597, at least 98% identical to the sequence given in SEQ ID NO:597, at least 97% identical to the sequence given in SEQ ID NO:597, at least 96% identical to the sequence given in SEQ ID NO:597, at least 95% identical to the sequence given in SEQ ID NO:597, at least 90% identical to the sequence given in SEQ ID NO:597, at least 85% identical to the sequence given in SEQ ID NO:597, or at least 80% identical to the sequence given in SEQ ID NO:597.

In an embodiment, a CCR of the present invention includes an IL-12R1 intracellular domain, encoded by a nucleotide comprising the sequence of SEQ ID NO:598, or a sequence that is at least 99% identical to the sequence given in SEQ ID NO:598, at least 98% identical to the sequence given in SEQ ID NO:598, at least 97% identical to the sequence given in SEQ ID NO:598, at least 96% identical to the sequence given in SEQ ID NO:598, at least 95% identical to the sequence given in SEQ ID NO:598, at least 90% identical to the sequence given in SEQ ID NO:598, at least 85% identical to the sequence given in SEQ ID NO:598, or at least 80% identical to the sequence given in SEQ ID NO:598.

In an embodiment, a CCR of the present invention includes an IL-12R2 intracellular domain, encoded by a nucleotide comprising the sequence of SEQ ID NO:599, or a sequence that is at least 99% identical to the sequence given in SEQ ID NO:599, at least 98% identical to the sequence given in SEQ ID NO:599, at least 97% identical to the sequence given in SEQ ID NO:599, at least 96% identical to the sequence given in SEQ ID NO:599, at least 95% identical to the sequence given in SEQ ID NO:599, at least 90% identical to the sequence given in SEQ ID NO:599, at least 85% identical to the sequence given in SEQ ID NO:599, or at least 80% identical to the sequence given in SEQ ID NO:599.

In an embodiment, a CCR of the present invention includes an IL-15Rα intracellular domain, encoded by a nucleotide comprising the sequence of SEQ ID NO:600, or a sequence that is at least 99% identical to the sequence given in SEQ ID NO:600, at least 98% identical to the sequence given in SEQ ID NO:600, at least 97% identical to the sequence given in SEQ ID NO:600, at least 96% identical to the sequence given in SEQ ID NO:600, at least 95% identical to the sequence given in SEQ ID NO:600, at least 90% identical to the sequence given in SEQ ID NO:600, at least 85% identical to the sequence given in SEQ ID NO:600, or at least 80% identical to the sequence given in SEQ ID NO:600.

In an embodiment, a CCR of the present invention includes an IL-21R intracellular domain, encoded by a nucleotide comprising the sequence of SEQ ID NO:601, or a sequence that is at least 99% identical to the sequence given in SEQ ID NO:601, at least 98% identical to the sequence given in SEQ ID NO:601, at least 97% identical to the sequence given in SEQ ID NO:601, at least 96% identical to the sequence given in SEQ ID NO:601, at least 95% identical to the sequence given in SEQ ID NO:601, at least 90% identical to the sequence given in SEQ ID NO:601, at least 85% identical to the sequence given in SEQ ID NO:601, or at least 80% identical to the sequence given in SEQ ID NO:601.

In an embodiment, a CCR of the present invention includes an IL-21R intracellular domain, encoded by a nucleotide comprising the sequence of SEQ ID NO:602, or a sequence that is at least 99% identical to the sequence given in SEQ ID NO:602, at least 98% identical to the sequence given in SEQ ID NO:602, at least 97% identical to the sequence given in SEQ ID NO:602, at least 96% identical to the sequence given in SEQ ID NO:602, at least 95% identical to the sequence given in SEQ ID NO:602, at least 90% identical to the sequence given in SEQ ID NO:602, at least 85% identical to the sequence given in SEQ ID NO:602, or at least 80% identical to the sequence given in SEQ ID NO:602.

In some embodiments, a CCR of the present invention includes an IL-21RAP (interleukin 18 receptor accessory protein) intracellular domain. The IL-18RAP domain is described in the examples below and is also disclosed in U.S. Patent Application Publication No. US 2019/0350974 A1, the disclosure of which is incorporated by reference herein. In an embodiment, a CCR of the present invention includes an IL-18RAP intracellular domain, encoded by a nucleotide comprising the sequence of SEQ ID NO:10 in U.S. Patent Application Publication No. US 2019/0350974 A1, or a sequence that is at least 99% identical to that sequence, at least 98% identical to that sequence, at least 97% identical to that sequence, at least 96% identical to the that sequence, at least 95% identical to that sequence, at least 90% identical to that sequence, at least 85% identical to that sequence, or at least 80% identical to that sequence.

In some embodiments, the intracellular domain may be linked to the transmembrane domain through a linker encoded by a nucleotide comprising the sequence of SED ID NO:603. In some embodiments, the intracellular domain may be extended to include the transmembrane domain and a short portion (e.g., 1 to 15 amino acids in length) of the extracellular domain, and then operatively linked to the remainder of the extracellular binding domain.

In an embodiment, a CCR of the present invention comprises an intracellular or costimulatory domain that includes a STAT3 signaling domain. In an embodiment, a CCR of the present invention comprises an intracellular domain that includes a JAK-STAT pathway signaling domain. Suitable STAT3 and JAK-STAT domains are described in Kagoya, et al., Nature Med. 2018, 24, 352-359 and U.S. Pat. No. 10,822,392, the disclosures of each of which are incorporated by reference herein.

In an embodiment, a CCR of the present invention comprises an intracellular or costimulatory domain that includes a CD40 ligand (CD40L or CD154) signaling domain. Suitable CD40L signaling domains are described in Aloui, et al., Int. J. Mol. Sci. 2014, 15(12), 22342-22364 and U.S. Pat. No. 10,287,354, the disclosures of each of which are incorporated by reference herein.

### D. Gene Expression Methods

In some embodiments, a method of genetically modifying a population of TILs to express CCRs or chemokine receptors includes the step of stable incorporation of genes for production of one or more proteins. In an embodiment, a method of genetically modifying a population of TILs includes the step of viral transduction. In an embodiment, a method of genetically modifying a population of TILs includes the step of retroviral transduction. In an embodiment, a method of genetically modifying a population of TILs includes the step of gamma-retroviral transduction. In an embodiment, a method of genetically modifying a population of TILs includes the step of adenoviral transduction. In an embodiment, a method of genetically modifying a population of TILs includes the step of adeno-associated viral transduction. In an embodiment, a method of genetically modifying a population of TILs includes the step of herpes simplex viral transduction. In an embodiment, a method of genetically modifying a population of TILs includes the step of poxvirus viral transduction. In some embodiments, a method of genetically modifying a population of TILs includes the step of lentiviral transduction, including lentiviral transduction using human immunodeficiency virus (HIV), including HIV-1. Lentiviral transduction systems and other suitable viral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. Nos. 5,350,674; 5,585,362; and 6,627,442, the disclosures of each of which are incorporated by reference herein. In an embodiment, a method of genetically modifying a population of TILs includes the step of gamma-retroviral transduction. Gamma-retroviral transduction systems are known in the art and are described, e.g., Cepko and Pear, Cur. Prot. Mol. Biol. 1996, 9.9.1-9.9.16, Hawley, et al., Gene Ther. 1994, 1, 136-38; the disclosure of which is incorporated by reference herein. In an embodiment, a pQCXIX retroviral vector is used to genetically modifying a population of TILs to express CCRs or chemokine receptors of the present invention. Other viral systems known in the art may similarly be employed to modify a population of TILs to stably or transiently express CCRs.

In some embodiments, a method of genetically modifying a population of TILs to express CCRs includes the step of preparing a lentiviral vector derived from at least a portion of a lentivirus genome, including especially a self-inactivating lentiviral vector as provided in Milone, et al., Mol. Ther. 2009, 17, 1453-1464. Other examples of lentivirus vectors that may be used in the clinic, include but are not limited to, e.g., the LENTrVECTOR™ gene delivery technology from Oxford BioMedica, the LENTIMAX™ vector system from Lentigen, and similar systems. In some embodiments, the lentiviral vector carrying the transgene is combined with a vesicular stomatitis virus glycoprotein (VSV-G) plasmid, which causes expression of a rhabdovirus envelope protein that binds to ubiquitous phospholipid components of the plasma membrane rather than to specific cell surface receptors and plasmids. In some embodiments, the lentiviral vector carrying the transgene is combined with Gag/Pol and Rev packaging plasmids. In some embodiments, lentiviral packaging is performing using a 293T cell line, such as a HEK293T cell line, or a variant, derivative, or progeny thereof.

In an embodiment, the viral or lentiviral vector backbone is PGEM.64A, as described in Zhao, et al., Mol. Ther. 2006, 13, 151-9, the disclosure of which is incorporated by reference herein. In an embodiment, the lentiviral vector backbone is pFUGW, as described in Lois, et al., Science 2002, 295, 868-72, the disclosure of which is incorporated by reference herein. In an embodiment, the lentiviral vector comprises a bovine growth hormone polyA sequence to drive expression of transgenes. In an embodiment, the lentiviral vector comprises a Kozak ribosomal initiation sequence. In an embodiment, the lentiviral vector comprises a woodchuck hepatitis virus posttranscriptional regulatory element (WPRE). In an embodiment the lentiviral vector comprises long terminal repeat (LTR) sequences derived from a pFUGW plasmid. In an embodiment, the viral or lentiviral vector backbone is pRRLSIN. In an embodiment, the viral or lentiviral vector backbone is pLenti. In an embodiment, the viral or retroviral vector backbone is pQCXIX.

In an embodiment, the viral or lentiviral vector backbone further comprises a promoter. In an embodiment, the promoter is a human elongation growth factor-1, or EF-1, promoter. In an embodiment, the promoter is an EF-1α. (also known as EF-1α or EF-1 alpha) promoter. In an embodiment, the promoter is the EF-1 promoter of SEQ ID NO:604 or a or functional portion or functional variant thereof. In an embodiment, the promoter is the immediate early cytomegalovirus (CMV) promoter sequence, which is a strong constitutive promoter sequence capable of driving high levels of expression of any polynucleotide sequence operatively linked thereto. In an embodiment, the promoter is the CMV promoter of SEQ ID NO:605 or a or functional portion or functional variant thereof. In an embodiment, the viral vector backbone further comprises a promoter, wherein the promotor is a murine embryonic stem cell virus (MSCV) promoter. In an embodiment, the promoter is the MSCV promoter of SEQ ID NO:606 or a or functional portion or functional variant thereof. In an embodiment, the viral or lentiviral vector backbone further comprises a promoter, wherein the promotor is a nuclear factor of activated T cells (NFAT) promoter. Suitable NFAT promoters are described in U.S. Pat. No. 8,556,882 and Merlet, et al., Gene Therapy 2013, 20, 248-254, the disclosures of which are incorporated by reference herein, and which may include one or more NFAT binding motifs, including NFAT1, NFAT2, NFAT3, and NFAT4 responsive elements. In an embodiment, the NFAT promoter comprises at least one, at least two, at least three, at least four, at least five, at least six, at least seven, at least eight, at least nine, at least ten, at least eleven, or at least twelve binding motifs. In an embodiment, the NFAT promoter comprises up to twelve binding motifs. In an embodiment, the NFAT promoter comprises four, five, six, or seven binding motifs. In an embodiment, the NFAT promoter comprises six binding motifs. In an embodiment, the promoter is the NFAT promoter of SEQ ID NO:607 or a or functional portion or functional variant thereof.

In an embodiment, the promoter is selected from the group consisting of an EF-1 promoter, a simian virus 40 (SV40) early promoter, a mouse mammary tumor virus (MMTV), a human immunodeficiency virus (HIV) LTR promoter, a MSCV promoter, a NFAT promoter, a Moloney murine leukemia virus (MoMuLV) promoter, an avian leukemia virus promoter, an Epstein-Barr virus immediate early promoter, a Rous sarcoma virus promoter, an actin promoter, a myosin promoter, a hemoglobin promoter, and a creatine kinase promoter. In an embodiment, the promoter is a constitutive promoter. In an embodiment, the promoter is an inducible promoter, which in some embodiments may be capable of turning on expression of the polynucleotide sequence which it is operatively linked when such expression is desired, or turning off the expression when expression is not desired. In an embodiment, the inducible promoter is selected from the group consisting of a metallothionine promoter, a glucocorticoid promoter, a progesterone promoter, and a tetracycline promoter. Exemplary, non-limiting sequences of suitable promoters are provided in Table 60.

TABLE 60
Nucleotide sequences of exemplary promoters.

| Identifier | Sequence (One-Letter Nucleotide Symbols) | |
|---|---|---|
| SEQ ID NO: 604 | GTGAGGCTCC GGTGCCCGTC AGTGGGCAGA GCGCACATCG CCCACAGTCC CCGAGAAGTT | 60 |
| EF1 promoter | GGGGGGAGGG GTCGGCAATT GAACCGGTGC CTAGAGAAGG TGGCGCGGGG TAAACTGGGA | 120 |
| | AAGTGATGTC GTGTACTGGC TCCGCCTTTT TCCCGAGGGT GGGGGAGAAC CGTATATAAG | 180 |
| | TGCAGTAGTC GCCGTGAACG TTCTTTTTCG CAACGGGTTT GCCGCCAGAA CACAGGTAAG | 240 |
| | TGCCGTGTGT GGTTCCCGCG GGCCTGGCCT CTTTACGGGT TATGGCCCTT GCGTGCCTTG | 300 |
| | AATTACTTCC ACCTGGCTGC AGTACGTGAT TCTTGATCCC GAGCTTCGGG TTGGAAGTGG | 360 |
| | GTGGGAGAGT TCGAGGCCTT GCGCTTAAGG AGCCCCTTCG CCTCGTGCTT GAGTTGAGGC | 420 |
| | CTGGCCTGGG CGCTGGGGCC GCCGCGTGCG AATCTGGTGG CACCTTCGCG CCTGTCTCGC | 480 |
| | TGCTTTCGAT AAGTCTCTAG CCATTTAAAA TTTTTGATGA CCTGCTGCGA CGCTTTTTTT | 540 |
| | CTGGCAAGAT AGTCTTGTAA ATGCGGGCCA AGATCTGCAC ACTGGTATTT CGGTTTTTGG | 600 |
| | GGCCGCGGGC GGCGACGGGG CCCGTGCGTC CCAGCGCACA TGTTCGGCGA GGCGGGGCCT | 660 |
| | GCGAGCGCGG CCACCGAGAA TCGGACGGGG GTAGTCTCAA GCTGGCCGGC CTGCTCTGGT | 720 |
| | GCCTGGCCTC GCGCCGCCGT GTATCGCCCC GCCCTGGGCG GCAAGGCTGG CCCGGTCGGC | 780 |
| | ACCAGTTGCG TGAGCGGAAA GATGGCCGCT TCCCGGCCCT GCTGCAGGGA GCTCAAAATG | 840 |
| | GAGGACGCGG CGCTCGGGAG AGCGGGCGGG TGAGTCACCC ACACAAAGGA AAAGGGCCTT | 900 |
| | TCCGTCCTCA GCCGTCGCTT CATGTGACTC CACGGAGTAC CGGGCGCCGT CCAGGCACCT | 960 |
| | CGATTAGTTC TCGAGCTTTT GGAGTACGTC GTCTTTAGGT TGGGGGGAGG GGTTTTATGC | 1020 |
| | GATGGAGTTT CCCCACACTG AGTGGGTGGA GACTGAAGTT AGGCCAGCTT GGCACTTGAT | 1080 |
| | GTAATTCTCC TTGGAATTTG CCCTTTTTGA GTTTGGATCT TGGTTCATTC TCAAGCCTCA | 1140 |
| | GACAGTGGTT CAAAGTTTTT TTCTTCCATT TCAGGTGTCG TGA | 1183 |
| SEQ ID NO: 605 | CGTTACATAA CTTACGGTAA ATGGCCCGCC TGGCTGACCG CCCAACGACC CCCGCCCATT | 60 |
| CMV promoter | GACGTCAATA ATGACGTATG TTCCCATAGT AACGCCAATA GGGACTTTCC ATTGACGTCA | 120 |
| | ATGGGTGGAG TATTTACGGT AAACTGCCCA CTTGGCAGTA CATCAAGTGT ATCATATGCC | 180 |
| | AAGTACGCCC CCTATTGACG TCAATGACGG TAAATGGCCC GCCTGGCATT ATGCCCAGTA | 240 |
| | CATGACCTTA TGGGACTTTC CTACTTGGCA GTACATCTAC GTATTAGTCA TCGCTATTAC | 300 |
| | CATGGTGATG CGGTTTTGGC AGTACATCAA TGGGCGTGGA TAGCGGTTTG ACTCACGGGG | 360 |
| | ATTTCCAAGT CTCCACCCCA TTGACGTCAA TGGGAGTTTG TTTTGGCACC AAAATCAACG | 420 |
| | GGACTTTCCA AAATGTCGTA ACAACTCCGC CCCATTGACG CAAATGGGCG GTAGGCGTGT | 480 |
| | ACGGTGGGAG GTCTATATAA GCAGAGCT | 508 |
| SEQ ID NO: 606 | TGAAAGACCC CACCTGTAGG TTTGGCAAGC TAGCTTAAGT AACGCCATTT TGCAAGGCAT | 60 |
| MSCV promoter | GGAAAATACA TAACTGAGAA TAGAGAAGTT CAGATCAAGG TTAGGAACAG AGAGACAGCA | 120 |
| | GAATATGGGC CAAACAGGAT ATCTGTGGTA AGCAGTTCCT GCCCCGGCTC AGGGCCAAGA | 180 |
| | ACAGATGGTC CCCAGATGCG GTCCCGCCCT CAGCAGTTTC TAGAGAACCA TCAGATGTTT | 240 |
| | CCAGGGTGCC CCAAGGACCT GAAATGACCC TGTGCCTTAT TTGAACTAAC CAATCAGTTC | 300 |
| | GCTTCTCGCT TCTGTTCGCG CGCTTCTGCT CCCCGAGCTC AATAAAAGAG CCCACAACCC | 360 |
| | CTCACTCGGC GCGCCAGTCC TCCGATAGAC TGCGTCGCCC GGGTACCCGT ATTCCCAATA | 420 |
| | AAGCCTCTTG CTGTTTGCAT CCGAATCGTG GACTCGCTGA TCCTTGGGAG GGTCTCCTCA | 480 |
| | GATTGATTGA CTGCCCACCT CGGGGGTCTT TCATT | 515 |
| SEQ ID NO: 607 | TCGAGGTCGA CGGTATCGAT AAGCTTGATA TCGAATTAGG AGGAAAAACT GTTTCATACA | 60 |
| NFAT promoter | GAAGGCGTCA ATTAGGAGGA AAAACTGTTT CATACAGAAG GCGTCAATTA GGAGGAAAAA | 120 |
| | CTGTTTCATA CAGAAGGCGT CAATTGGTCC CATCGAATTA GGAGGAAAAA CTGTTTCATA | 180 |
| | CAGAAGGCGT CAATTAGGAG GAAAAACTGT TTCATACAGA AGGCGTCAAT TAGGAGGAAA | 240 |
| | AACTGTTTCA TACAGAAGGC GTCAATTGGT CCCGGGACAT TTTGACACCC CCATAATATT | 300 |
| | TTTCCAGAAT TAACAGTATA AATTGCATCT CTTGTTCAAG AGTTCCCTAT CACTCTCTTT | 360 |
| | AATCACTACT CACAGTAACC TCAACTCCTG | 390 |

In an embodiment, a promoter domain used with a vector encoding a CCR or chemokine receptor for the modification of TILs, MILs, or PBLs as described herein comprises the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 99% identical to the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 98% identical to the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 97% identical to the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 96% identical to the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 95% identical to the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 90% identical to the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 85% identical to the nucleotide sequence of SEQ ID NO:604. In an embodiment, a promoter domain used with a vector encoding a CCR or chemokine receptor for the modification of TILs, MILs, or PBLs as described herein comprises a nucleotide sequence that is at least 80% identical to the nucleotide sequence of SEQ ID NO:604. In an embodiment, including the foregoing embodiments, SEQ ID NO:604 is optimized to improve protein expression.

In an embodiment, a promoter domain used with a vector encoding a CCR or chemokine receptor for the modification of TILs, MILs, or PBLs as described herein comprises the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 99% identical to the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 98% identical to the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 97% identical to the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 96% identical to the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 95% identical to the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 90% identical to the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 85% identical to the nucleotide sequence of SEQ ID NO:605. In an embodiment, a promoter domain used with a vector encoding a CCR or chemokine receptor for the modification of TILs, MILs, or PBLs as described herein comprises a nucleotide sequence that is at least 80% identical to the nucleotide sequence of SEQ ID NO:605. In an embodiment, including the foregoing embodiments, SEQ ID NO:605 is optimized to improve protein expression.

In an embodiment, a promoter domain used with a vector encoding a CCR or chemokine receptor for the modification of TILs, MILs, or PBLs as described herein comprises the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 99% identical to the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 98% identical to the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 97% identical to the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 96% identical to the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 95% identical to the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 90% identical to the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 85% identical to the nucleotide sequence of SEQ ID NO:606. In an embodiment, a promoter domain used with a vector encoding a CCR or chemokine receptor for the modification of TILs, MILs, or PBLs as described herein comprises a nucleotide sequence that is at least 80% identical to the nucleotide sequence of SEQ ID NO:606. In an embodiment, including the foregoing embodiments, SEQ ID NO:606 is optimized to improve protein expression.

In an embodiment, a promoter domain used with a vector encoding a CCR or chemokine receptor for the modification of TILs, MILs, or PBLs as described herein comprises the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 99% identical to the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 98% identical to the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 97% identical to the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 96% identical to the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 95% identical to the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 90% identical to the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 85% identical to the nucleotide sequence of SEQ ID NO:607. In an embodiment, a promoter domain used with a vector encoding a CCR or chemokine receptor for the modification of TILs, MILs, or PBLs as described herein comprises a nucleotide sequence that is at least 80% identical to the nucleotide sequence of SEQ ID NO:607. In an embodiment, including the foregoing embodiments, SEQ ID NO:607 is optimized to improve protein expression.

In one embodiment, the vector is an in vitro transcribed vector, including a vector that transcribes RNA of a nucleic acid molecule described herein. In one embodiment, the nucleic acid sequence of the vector further comprises a polyadenylated or poly(A) tail comprising about 10, about 20, about 30, about 40, about 50, about 60, about 70, about 80, about 90, about 100, about 110, about 120, about 130, about 140, about 150, about 160, about 170, about 180, about 190, or about 200 adenosine bases. In one embodiment, the nucleic acid sequence in the vector further comprises a 3′ untranslated region (UTR) comprising at least one repeat of a 3′ UTR derived from human beta-globulin.

The present invention also includes an RNA construct that can be directly transfected into a cell. A method for generating mRNA for use in transfection involves in vitro transcription (IVT) of a template with specially designed primers, followed by poly(A) addition, to produce a construct containing 3′ and 5′ UTR, a 5′ cap and/or an internal ribosome entry site (IRES), the nucleic acid to be expressed, and a polyA tail, typically 50-2000 bases in length. RNA so produced can efficiently transfect different kinds of cells. In one embodiment, the template includes sequences for a CCR described herein. In an embodiment, an RNA CCR vector is transduced into a T cell by electroporation.

In an embodiment, the vector expression systems disclosed in U.S. Patent Application Publication No. US 2019/0298770 A1, substituted with appropriate transgenes encoding the CCRs of the present invention, may be used, and the disclosure of these vector expression systems is incorporated by reference herein.

In an embodiment, a method of genetically modifying a population of TILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, a CCR is transiently expressed by a population of TILs. In some embodiments, a CCR is transiently expressed by a population of TILs through electroporation of RNA. In an embodiment, a method of genetically modifying a population of TILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In an embodiment, the electroporation method is a sterile electroporation method. In an embodiment, the electroporation method is a pulsed electroporation method. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In an embodiment, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In an embodiment, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained. In an embodiment, a method of genetically modifying a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Nat. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In an embodiment, a method of genetically modifying a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Nat. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In an embodiment, a method of genetically modifying a population of TILs includes the step of transfection using methods described in U.S. Pat. Nos. 5,766,902; 6,025,337; 6,410,517; 6,475,994; and 7,189,705; the disclosures of each of which are incorporated by reference herein.

According to one embodiment, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2, and optionally OKT-3 (e.g., OKT-3 may be present in the culture medium beginning on the start date of the expansion process), to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing or replacing the cell culture medium of the second population of TILs with additional IL-2, media, optionally OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system; and
(g) at any time during steps (e) to (f), genetically modifying at least a portion of the TILs to stably or transiently express one or more chimeric costimulatory receptors.

As stated in step (g) of the embodiment described above, the gene modification process may be carried out at any time during the TIL expansion method, which means that the gene modification may be carried out on TILs before, during, or after any of the steps in the expansion method; for example, during any of steps (a)-(f) outlined in the method above, or before or after any of steps (a)-(f) outlined in the method above. According to certain embodiments, TILs are collected during the expansion method (e.g., the expansion method is “paused” for at least a portion of the TILs), and the collected TILs are subjected to a gene-modification process, and, in some cases, subsequently reintroduced back into the expansion method (e.g., back into the culture medium) to continue the expansion process, so that at least a portion of the therapeutic population of TILs that are eventually transferred to the infusion bag are permanently gene-edited. In an embodiment, the gene modification process may be carried out before expansion by activating TILs, performing a gene modification step on the activated TILs, and expanding the genetically modified TILs according to the processes described herein. In any of the foregoing embodiments, the gene modification process may cause the expression of a CCR described herein. It should be noted that alternative embodiments of the expansion process may differ from the method shown above. Alternative embodiments may not have the same steps (a)-(g), or may have a different number of steps. Regardless of the specific embodiment, the gene-editing process may be carried out at any time during the TIL expansion method. For example, alternative embodiments may include more than two rapid expansions, and gene-editing may be conducted on the TILs during a third or fourth expansion.

In some embodiments, the gene-editing process is carried out on TILs from one or more of a first population, a second population, and a third population, of the processes for TIL manufacture described herein. For example, gene-editing may be carried out on the first population of TILs, or on a portion of TILs collected from the first population, and following the gene-editing process those TILs may subsequently be placed back into the expansion process (e.g., back into the culture medium). Alternatively, gene-editing may be carried out on TILs from the second or third population, or on a portion of TILs collected from the second or third population, respectively, and following the gene-editing process those TILs may subsequently be placed back into the expansion process (e.g., back into the culture medium). According to another embodiment, gene-editing is performed while the TILs remain in a culture medium and while the expansion is being carried out.

In some embodiments, the gene-editing process is carried out on TILs from the first expansion, or TILs from the second expansion, or both. For example, during the first expansion or second expansion, gene-editing may be carried out on TILs that are collected from the culture medium, and following the gene-editing process those TILs may subsequently be placed back into the expansion method, e.g., by reintroducing them back into the culture medium.

In some embodiments, the gene-editing process is carried out on at least a portion of the TILs after the first expansion and before the second expansion. For example, after the first expansion, gene-editing may be carried out on TILs that are collected from the culture medium, and following the gene-editing process those TILs may subsequently be placed back into the expansion method, e.g., by reintroducing them back into the culture medium for the second expansion.

In some embodiments, the gene-editing process is carried out before step (c) (e.g., before, during, or after any of steps (a)-(b)), before step (d) (e.g., before, during, or after any of steps (a)-(c)), before step (e) (e.g., before, during, or after any of steps (a)-(d)), or before step (f) (e.g., before, during, or after any of steps (a)-(e)).

In some embodiments, the cell culture medium may comprise OKT-3 beginning on the start day (Day 0), or on Day 1 of the first expansion, such that the gene-editing is carried out on TILs after they have been exposed to OKT-3 in the cell culture medium on Day 0 and/or Day 1. In some embodiments, the cell culture medium comprises OKT-3 during the first expansion and/or during the second expansion, and the gene-editing is carried out before the OKT-3 is introduced into the cell culture medium. Alternatively, the cell culture medium may comprise OKT-3 during the first expansion and/or during the second expansion, and the gene-editing is carried out after the OKT-3 is introduced into the cell culture medium.

In some embodiments, the cell culture medium may comprise a 4-1BB agonist or OX40 agonist beginning on the start day (Day 0), or on Day 1 of the first expansion, such that the gene-editing is carried out on TILs after they have been exposed to a 4-1BB agonist or OX40 agonist in the cell culture medium on Day 0 and/or Day 1. In some embodiments, the cell culture medium comprises a 4-1BB agonist or OX40 agonist during the first expansion and/or during the second expansion, and the gene-editing is carried out before the 4-1BB agonist or OX40 agonist is introduced into the cell culture medium. In some embodiments, the cell culture medium may comprise a 4-1BB agonist or OX40 agonist during the first expansion and/or during the second expansion, and the gene-editing is carried out after the 4-1BB agonist or OX40 agonist is introduced into the cell culture medium.

In some embodiments, that the cell culture medium may comprise IL-2 beginning on the start day (Day 0), or on Day 1 of the first expansion, such that the gene-editing is carried out on TILs after they have been exposed to IL-2 in the cell culture medium on Day 0 and/or Day 1. According to another embodiment, the cell culture medium comprises IL-2 during the first expansion and/or during the second expansion, and the gene-editing is carried out before the IL-2 is introduced into the cell culture medium. Alternatively, the cell culture medium may comprise IL-2 during the first expansion and/or during the second expansion, and the gene-editing is carried out after the IL-2 is introduced into the cell culture medium.

As discussed above, one or more of OKT-3, a 4-1BB agonist and IL-2 may be included in the cell culture medium beginning on Day 0 or Day 1 of the first expansion. According to one embodiment, OKT-3 is included in the cell culture medium beginning on Day 0 or Day 1 of the first expansion, and/or a 4-1BB agonist is included in the cell culture medium beginning on Day 0 or Day 1 of the first expansion, and/or IL-2 is included in the cell culture medium beginning on Day 0 or Day 1 of the first expansion. According to an example, the cell culture medium comprises OKT-3 and a 4-1BB agonist beginning on Day 0 or Day 1 of the first expansion. According to another example, the cell culture medium comprises OKT-3, a 4-1BB agonist and IL-2 beginning on Day 0 or Day 1 of the first expansion. Of course, one or more of OKT-3, 4-1BB agonist and IL-2 may be added to the cell culture medium at one or more additional time points during the expansion process, as set forth in various embodiments described herein.

According to one embodiment, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments or a tumor digest, optionally after thawing cryopreserved multiple tumor fragments or cryopreserved tumor digest;
(b) adding the tumor fragments or tumor digest into a closed system;
(c) performing a first expansion by culturing the first population of TILs in a cell culture medium comprising IL-2 and optionally comprising a 4-1BB agonist antibody for about 2 to 5 days;
(d) adding OKT-3, to produce a second population of TILs, wherein the first expansion is performed in a closed container providing a first gas-permeable surface area, wherein the first expansion is performed for about 1 to 3 days to obtain the second population of TILs, wherein the second population of TILs is at least 50-fold greater in number than the first population of TILs, and wherein the transition from step (c) to step (d) occurs without opening the system;
(e) genetically modifying at least a portion of the TILs to stably or transiently express one or more chimeric costimulatory receptors, optionally by transferring such portion of the TILs to a temporary container;
(f) optionally resting the second population of TILs for about 1 day to about 5 days;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) cryopreserving the harvested TIL population using a dimethylsulfoxide-based cryopreservation medium.

According to one embodiment, the foregoing method may be used to provide an autologous harvested TIL population for the treatment of a human subject with cancer.

In some embodiments, a vector encoding a CCR and/or chemokine receptor for the modification of TILs, MILs, or PBLs as described herein comprises a nucleotide sequence that encodes a 2A self-cleaving peptide. Vectors encoding 2A self-cleaving peptides may be employed with the CCRs and chemokines of the present invention to induce ribosomal skipping during translation and generate polyproteins. Without being bound by theory, a vector encoding a CCR or chemokine receptor comprising a 2A self-cleaving peptide may, upon expression inside a cell, be cleaved into two proteins, such as two CCRs or a CCR and another protein. The amino acid sequences of exemplary and non-limiting 2A self-cleaving peptide domains are provided in Table 61. SEQ ID NO:608 is an amino acid sequence for a T2A self-cleaving peptide (derived from thosea asigna virus 2A), SEQ ID NO:609 is an amino acid sequence for a P2A self-cleaving peptide (derived from porcine teschovirus-1 2A), SEQ ID NO:610 is an amino acid sequence for a E2A self-cleaving peptide (derived from equine rhinitis A virus), and SEQ ID NO:611 is an amino acid sequence for a F2A self-cleaving peptide (derived from foot-and-mouth disease virus).

TABLE 61
Amino acid sequences of
exemplary 2A self-cleaving peptides.

| | Sequence (One-Letter | |
|---|---|---|
| Identifier | Amino Acid Symbols) | |
| SEQ ID NO: 608 | EGRGSLLTCG DVEENPGP | 18 |
| T2A self- | | |
| cleaving | | |
| peptide | | |
| SEQ ID NO: 609 | ATNFSLLKQA GDVEENPGP | 19 |
| P2A self- | | |
| cleaving | | |
| peptide | | |
| SEQ ID NO: 610 | QCTNYALLKL AGDVESNPGP | 20 |
| E2A self- | | |
| cleaving | | |
| peptide | | |
| SEQ ID NO: 611 | VKQTLNFDLL KLAGDVESNP GP | 22 |
| F2A self- | | |
| cleaving | | |
| peptide | | |
| SEQ ID NO: 612 | GSG | 3 |
| linker | | |

In an embodiment, a 2A self-cleaving peptide domain used with a vector encoding a CCR and/or a chemokine receptor for the modification of TILs, MILs, or PBLs as described herein comprises the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 99% identical to the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 98% identical to the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 97% identical to the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 96% identical to the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 95% identical to the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 90% identical to the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 85% identical to the amino acid sequence of SEQ ID NO:608. In an embodiment, a 2A self-cleaving peptide domain used with a vector encoding a CCR and/or a chemokine receptor for the modification of TILs, MILs, or PBLs as described herein comprises an amino acid sequence that is at least 80% identical to the amino acid sequence of SEQ ID NO:608.

In an embodiment, a 2A self-cleaving peptide domain used with a vector encoding a CCR and/or a chemokine receptor for the modification of TILs, MILs, or PBLs as described herein comprises the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 99% identical to the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 98% identical to the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 97% identical to the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 96% identical to the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 95% identical to the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 90% identical to the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 85% identical to the amino acid sequence of SEQ ID NO:609. In an embodiment, a 2A self-cleaving peptide domain used with a vector encoding a CCR and/or a chemokine receptor for the modification of TILs, MILs, or PBLs as described herein comprises an amino acid sequence that is at least 80% identical to the amino acid sequence of SEQ ID NO:609.

In an embodiment, a 2A self-cleaving peptide domain used with a vector encoding a CCR and/or a chemokine receptor for the modification of TILs, MILs, or PBLs as described herein comprises the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 99% identical to the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 98% identical to the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 97% identical to the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 96% identical to the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 95% identical to the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 90% identical to the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 85% identical to the amino acid sequence of SEQ ID NO:610. In an embodiment, a 2A self-cleaving peptide domain used with a vector encoding a CCR and/or a chemokine receptor for the modification of TILs, MILs, or PBLs as described herein comprises an amino acid sequence that is at least 80% identical to the amino acid sequence of SEQ ID NO:610.

In an embodiment, a 2A self-cleaving peptide domain used with a vector encoding a CCR and/or a chemokine receptor for the modification of TILs, MILs, or PBLs as described herein comprises the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 99% identical to the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 98% identical to the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 97% identical to the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 96% identical to the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 95% identical to the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 90% identical to the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 85% identical to the amino acid sequence of SEQ ID NO:611. In an embodiment, a 2A self-cleaving peptide domain used with a vector encoding a CCR and/or a chemokine receptor for the modification of TILs, MILs, or PBLs as described herein comprises an amino acid sequence that is at least 80% identical to the amino acid sequence of SEQ ID NO:611.

The foregoing 2A self-cleaving peptide domains may also be combined with a linker, such as a GSG linker (SEQ ID NO:612), at the N-terminus. Alternative linkers include SEQ ID NO:238, SEQ ID NO:239, SEQ ID NO:240, SEQ ID NO:241, SEQ ID NO:242, SEQ ID NO:243, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72 SEQ ID NO:74, conservative amino acid substitutions thereof, variants thereof, or other linkers known in the art, including those described in Bird, et al., Science 1988, 242, 423-426, the disclosures of which are incorporated by reference herein.

Exemplary, non-limiting nucleotide sequences of suitable 2A self-cleaving peptide domains are provided in Table 62.

TABLE 62
Nucleotide sequences of exemplary 2A self-cleaving peptides.

| Identifier | Sequence (One-Letter Nucleotide Symbols) | |
|---|---|---|
| SEQ ID NO: 613 | GGAAGCGGAG AGGGCAGGGG AAGTCTTCTA ACATGCGGGG ACGTGGAGGA AAATCCCGGC | 60 |
| T2A self- | CCC | 63 |
| cleaving | | |
| peptide from | | |
| thosea asigna | | |
| virus 2A | | |
| SEQ ID NO: 614 | GGAAGCGGAG CCACGAACTT CTCTCTGTTA AAGCAAGCAG GAGATGTTGA AGAAAACCCC | 60 |
| P2A self- | GGGCCT | 66 |
| cleaving | | |
| peptide from | | |
| porcine | | |
| teschovirus-1 | | |
| SEQ ID NO: 615 | GGAAGCGGAC AGTGTACTAA TTATGCTCTC TTGAAATTGG CTGGAGATGT TGAGAGCAAC | 60 |
| E2A self- | CCAGGTCCC | 69 |
| cleaving | | |
| peptide from | | |
| equine rhinitis | | |
| A virus | | |
| SEQ ID NO: 616 | GGAAGCGGAG TGAAACAGAC TTTGAATTTT GACCTTCTGA AGTTGGCAGG AGACGTTGAG | 60 |
| F2A self- | TCCAACCCTG GGCCC | 75 |
| cleaving | | |
| peptide from | | |
| foot-and-mouth | | |
| disease virus | | |
| SEQ ID NO: 617 | GCCCCTCTCC CTCCCCCCCC CCTAACGTTA CTGGCCGAAG CCGCTTGGAA TAAGGCCGGT | 60 |
| IRES from | GTGCGTTTGT CTATATGTTA TTTTCCACCA TATTGCCGTC TTTTGGCAAT GTGAGGGCCC | 120 |
| encephalomyocarditis | GGAAACCTGG CCCTGTCTTC TTGACGAGCA TTCCTAGGGG TCTTTCCCCT CTCGCCAAAG | 180 |
| virus | GAATGCAAGG TCTGETGAAT GTCGTGAAGG AAGCAGTTCC TCTGGAAGCT TCTTGAAGAC | 240 |
| internal | AAACAACGTC TGTAGCGACC CTTTGCAGGC AGCGGAACCC CCCACCTGGC GACAGGTGCC | 300 |
| ribosome entry | TCTGCGGCCA AAAGCCACGT GTATAAGATA CACCTGCAAA GGCGGCACAA CCCCAGTGCC | 360 |
| site | ACGTTGTGAG TTGGATAGTT GTGGAAAGAG TCAAATGGCT CTCCTCAAGC GTATTCAACA | 420 |
| | AGGGGCTGAA GGATGCCCAG AAGGTACCCC ATTGTATGGG ATCTGATCTG GGGCCTCGGT | 480 |
| | GCACATGCTT TACATGTGTT TAGTCGAGGT TAAAAAAACG TCTAGGCCCC CCGAACCACG | 540 |
| | GGGACGTGGT TTTCCTTTGA AAAACACGAT GATAATATGG CCACAACC | 588 |

In an embodiment, a 2A self-cleaving peptide domain used with a vector encoding a CCR and/or a chemokine receptor for the modification of TILs, MILs, or PBLs as described herein comprises the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 99% identical to the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 98% identical to the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 97% identical to the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 96% identical to the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 95% identical to the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 90% identical to the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 85% identical to the nucleotide sequence of SEQ ID NO:613. In an embodiment, a 2A self-cleaving peptide domain used with a vector encoding a CCR and/or a chemokine receptor for the modification of TILs, MILs, or PBLs as described herein comprises a nucleotide sequence that is at least 80% identical to the nucleotide sequence of SEQ ID NO:613. In an embodiment, including the foregoing embodiments, SEQ ID NO:613 is optimized to improve protein expression.

In an embodiment, a 2A self-cleaving peptide domain used with a vector encoding a CCR and/or a chemokine receptor for the modification of TILs, MILs, or PBLs as described herein comprises the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 99% identical to the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 98% identical to the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 97% identical to the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 96% identical to the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 95% identical to the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 90% identical to the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 85% identical to the nucleotide sequence of SEQ ID NO:614. In an embodiment, a 2A self-cleaving peptide domain used with a vector encoding a CCR and/or a chemokine receptor for the modification of TILs, MILs, or PBLs as described herein comprises a nucleotide sequence that is at least 80% identical to the nucleotide sequence of SEQ ID NO:614. In an embodiment, including the foregoing embodiments, SEQ ID NO:614 is optimized to improve protein expression.

In an embodiment, a 2A self-cleaving peptide domain used with a vector encoding a CCR and/or a chemokine receptor for the modification of TILs, MILs, or PBLs as described herein comprises the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 99% identical to the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 98% identical to the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 97% identical to the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 96% identical to the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 95% identical to the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 90% identical to the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 85% identical to the nucleotide sequence of SEQ ID NO:615. In an embodiment, a 2A self-cleaving peptide domain used with a vector encoding a CCR and/or a chemokine receptor for the modification of TILs, MILs, or PBLs as described herein comprises a nucleotide sequence that is at least 80% identical to the nucleotide sequence of SEQ ID NO:615. In an embodiment, including the foregoing embodiments, SEQ ID NO:615 is optimized to improve protein expression.

In an embodiment, a 2A self-cleaving peptide domain used with a vector encoding a CCR and/or a chemokine receptor for the modification of TILs, MILs, or PBLs as described herein comprises the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 99% identical to the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 98% identical to the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 97% identical to the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 96% identical to the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 95% identical to the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 90% identical to the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 85% identical to the nucleotide sequence of SEQ ID NO:616. In an embodiment, a 2A self-cleaving peptide domain used with a vector encoding a CCR and/or a chemokine receptor for the modification of TILs, MILs, or PBLs as described herein comprises a nucleotide sequence that is at least 80% identical to the nucleotide sequence of SEQ ID NO:616. In an embodiment, including the foregoing embodiments, SEQ ID NO:616 is optimized to improve protein expression.

In an embodiment, a vector encoding a CCR and/or a chemokine receptor of the present invention comprises an IRES domain. Suitable IRES domains are known in the art. In an embodiment, a vector encoding a CCR and/or a chemokine receptor of the present invention comprises the nucleotide sequence of SEQ ID NO:617. In an embodiment, a vector encoding a CCR and/or a chemokine receptor for the modification of TILs, MILs, or PBLs as described herein comprises a nucleotide sequence that is at least 99% identical to the nucleotide sequence of SEQ ID NO:617. In an embodiment, a vector encoding a CCR and/or a chemokine receptor for the modification of TILs, MILs, or PBLs as described herein comprises a nucleotide sequence that is at least 98% identical to the nucleotide sequence of SEQ ID NO:617. In an embodiment, a vector encoding a CCR and/or a chemokine receptor for the modification of TILs, MILs, or PBLs as described herein comprises a nucleotide sequence that is at least 97% identical to the nucleotide sequence of SEQ ID NO:617. In an embodiment, a vector encoding a CCR and/or a chemokine receptor for the modification of TILs, MILs, or PBLs as described herein comprises a nucleotide sequence that is at least 96% identical to the nucleotide sequence of SEQ ID NO:617. In an embodiment, a vector encoding a CCR and/or a chemokine receptor for the modification of TILs, MILs, or PBLs as described herein comprises a nucleotide sequence that is at least 95% identical to the nucleotide sequence of SEQ ID NO:617. In an embodiment, a vector encoding a CCR and/or a chemokine receptor for the modification of TILs, MILs, or PBLs as described herein comprises a nucleotide sequence that is at least 90% identical to the nucleotide sequence of SEQ ID NO:617. In an embodiment, a vector encoding a CCR and/or a chemokine receptor for the modification of TILs, MILs, or PBLs as described herein comprises a nucleotide sequence that is at least 85% identical to the nucleotide sequence of SEQ ID NO:617. In an embodiment, a vector encoding a CCR and/or a chemokine receptor for the modification of TILs, MILs, or PBLs as described herein comprises a nucleotide sequence that is at least 80% identical to the nucleotide sequence of SEQ ID NO:617. In an embodiment, including the foregoing embodiments, SEQ ID NO:617 is optimized to improve protein expression. In an embodiment, including the foregoing embodiments, SEQ ID NO:617 is optimized to improve protein expression. Other suitable IRES domains are described in Bochkov and Palmenberg, Biotechniques 2006, 41(3), 283, the disclosures of which are incorporated by reference herein.

### E. CCR Constructs

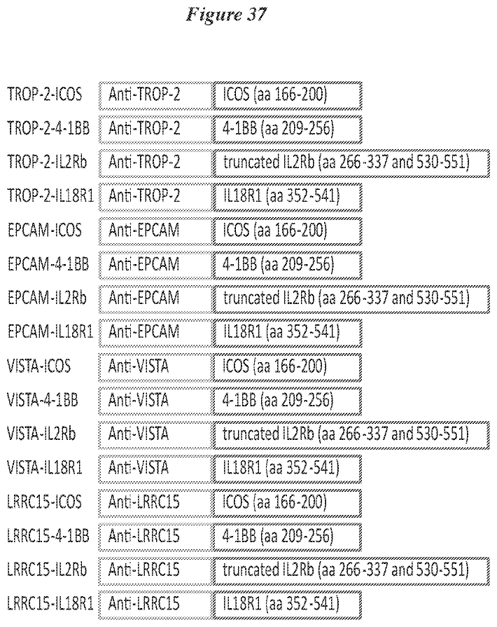

The foregoing extracellular and intracellular domains may be combined, and optionally further combined by a transmembrane domain, to provide CCRs suitable for use with the TILs of the present invention. Several exemplary CCR constructs of the present invention have been previously discussed, and other exemplary CCR constructs are depicted in FIG. 37 or described herein, each of which is an embodiment of the present invention.

In some embodiments, a CCR of the present invention comprises an extracellular domain selected from the group consisting of a DLL3-domain, a PD-1 domain, a FAS binding domain, a TGFβ binding domain, a PD-L1 scFv binding domain, a CEA scFv binding domain, a CD73 scFv binding domain, a TROP-2 scFv binding domain, an EPCAM scFv binding domain, a tissue factor scFv binding domain, a FAP scFv binding domain, an LFA-1 scFv binding domain, a VISTA scFv binding domain, and a LLRC15 scFv binding domain.

In some embodiments, a CCR of the present invention comprises an extracellular domain that binds to a molecule selected from the group consisting of DLL3, CD19, CD20, CD22, CD24, CD33, CD38, CD39, CD73, CD123, CD138, CD228, LRRC15, CEA, FRα, EPCAM, PD-L1, PSMA, gp100, MUC1, MCSP, EGFR, GD2, TROP-2, GPC3, MICA, MICB, VISTA, ULBP, HER2, MCM5, FAP, 5T4, LFA-1, B7-H3, FAS, TGFβ, TGFβRII, and MUC16.

In some embodiments, a CCR of the present invention comprises: (i) an extracellular domain selected from the group consisting of a DLL3 domain, a PD-1 domain, a PD-L1 scFv binding domain, a CEA scFv binding domain, a CD73 scFv binding domain, a TROP-2 scFv binding domain, an EPCAM scFv binding domain, a tissue factor scFv binding domain, an LFA-1 scFv binding domain, a FAP scFv binding domain, a VISTA scFv binding domain, and a LLRC15 scFv binding domain, and (ii) an intracellular domain selected from the group consisting of CD28, CD134 (OX40), CD278 (ICOS), CD137 (4-1BB), CD27, IL-2Rβ, IL-2Rγ, IL-18R1, IL-18RAP, IL-7Rα, IL-12R1, IL-12R2, IL-15Rα, and IL-21R.

In some embodiments, a CCR of the present invention comprises: (i) an extracellular domain that binds to a molecule selected from the group consisting of CD19, CD20, CD22, CD24, CD33, CD38, CD39, CD73, CD123, CD138, CD228, LRRC15, CEA, FRα, EPCAM, DLL3, PD-L1, PSMA, gp100, MUC1, MCSP, EGFR, GD2, TROP-2, GPC3, MICA, MICB, VISTA, ULBP, HER2, MCM5, FAP, 5T4, LFA-1, B7-H3, and MUC16, and (ii) an intracellular domain selected from the group consisting of CD28, CD134 (OX40), CD278 (ICOS), CD137 (4-1BB), CD27, IL-2Rβ, IL-2Rγ, IL-18R1, IL-18RAP, IL-7Rα, IL-12R1, IL-12R2, IL-15Rα, and IL-21R.

In some embodiments, a CCR of the present invention is a protein that comprises: (i) an extracellular domain selected from the group consisting of a DLL3 domain, a PD-1 domain, a PD-L1 scFv binding domain, a CEA scFv binding domain, a CD73 scFv binding domain, a TROP-2 scFv binding domain, an EPCAM scFv binding domain, a tissue factor scFv binding domain, an LFA-1 scFv binding domain, a FAP scFv binding domain, a VISTA scFv binding domain, and a LLRC15 scFv binding domain, and (ii) an intracellular domain selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof. In some embodiments, the extracellular domain and intracellular domain are operatively linked. In some embodiments, the extracellular domain and intracellular domain are linked by a linker domain.

In some embodiments, a CCR of the present invention is a protein that comprises: (i) an extracellular domain selected from the group consisting of a DLL3 domain, a PD-1 domain, a PD-L1 scFv binding domain, a CEA scFv binding domain, a CD73 scFv binding domain, a TROP-2 scFv binding domain, an EPCAM scFv binding domain, a tissue factor scFv binding domain, an LFA-1 scFv binding domain, a FAP scFv binding domain, a VISTA scFv binding domain, and a LLRC15 scFv binding domain, (ii) a transmembrane domain selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-18 domain, an IL-2Rα domain, an IL-2Rβ domain, and an IL-2Rγ domain, and (iii) an intracellular domain selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-18RAP domain, an IL-7Rα domain, an IL-12R1 domain, an IL-12R2 domain, an IL-15Rα domain, an IL-21R domain, and combinations thereof. In some embodiments, the extracellular domain and transmembrane domain are operatively linked, and the transmembrane domain and intracellular domain are operatively linked. In some embodiments, the extracellular domain, transmembrane domain and intracellular domain are each linked to one another by a linker domain.

In some embodiments, a CCR of the present invention is a protein that comprises: (i) an extracellular domain selected from the group consisting of a DLL3 domain, a PD-1 domain, a PD-L1 scFv binding domain, a CEA scFv binding domain, a CD73 scFv binding domain, a TROP-2 scFv binding domain, an EPCAM scFv binding domain, a tissue factor scFv binding domain, an LFA-1 scFv binding domain, a FAP scFv binding domain, a VISTA scFv binding domain, and a LLRC15 scFv binding domain, (ii) a transmembrane domain selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, (iii) a hinge protein domain selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain, and (iv) an intracellular domain selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-18RAP domain, an IL-7Rα domain, an IL-12R1 domain, an IL-12R2 domain, an IL-15Rα domain, an IL-21R domain, and combinations thereof. In some embodiments, the extracellular domain and hinge domain are operatively linked, the hinge domain and transmembrane domain are operatively linked, and the transmembrane domain and intracellular domain are operatively linked. In some embodiments, the extracellular domain, transmembrane domain and intracellular domain are each linked to one another by a linker domain.

In some embodiments, the present invention includes a TIL, MIL, or PBL that expresses a CCR that comprises a protein sequence that comprises a PD-1 domain and a PD-1 transmembrane domain. In an embodiment, the present invention includes a TIL, MIL or PBL that expresses a protein sequence comprising SEQ ID NO:244 operatively linked to SEQ ID NO:245. In an embodiment, the present invention includes a TIL, MIL or PBL that expresses a protein sequence comprising SEQ ID NO:244 linked through a linker domain to SEQ ID NO:245. In an embodiment, the present invention includes a TIL, MIL or PBL that expresses a protein sequence comprising SEQ ID NO:244 operatively linked to SEQ ID NO:245, which is further operatively linked to an intracellular domain. In an embodiment, the present invention includes a TIL, MIL or PBL that expresses a protein sequence comprising SEQ ID NO:244 operatively linked to SEQ ID NO:245, which is further operatively linked to an intracellular domain selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof.

In some embodiments, the present invention includes a TIL, MIL, or PBL that expresses a CCR that comprises a protein sequence that comprises a PD-1 domain and a CD28 transmembrane domain. In an embodiment, the present invention includes a TIL, MIL or PBL that expresses a protein sequence comprising SEQ ID NO:244 operatively linked to SEQ ID NO:246. In an embodiment, the present invention includes a TIL, MIL or PBL that expresses a protein sequence comprising SEQ ID NO:244 linked through a linker domain to SEQ ID NO:246. In an embodiment, the present invention includes a TIL, MIL or PBL that expresses a protein sequence comprising SEQ ID NO:244 operatively linked to SEQ ID NO:246, which is further operatively linked to an intracellular domain. In an embodiment, the present invention includes a TIL, MIL or PBL that expresses a protein sequence comprising SEQ ID NO:244 operatively linked to SEQ ID NO:246, which is further operatively linked to an intracellular domain selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-7Rα domain, an IL-12R1 domain, an IL-12R2 domain, an IL-15Rα domain, an IL-21R domain, and combinations thereof.

In some embodiments, the present invention includes a TIL, MIL, or PBL that expresses a CCR that comprises a protein sequence that comprises a PD-L1 binding (anti-PD-L1) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a PD-L1 binding (anti-PD-L1) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a PD-L1 binding (anti-PD-L1) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a PD-L1 binding (anti-PD-L1) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a PD-L1 binding (anti-PD-L1) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a PD-L1 binding (anti-PD-L1) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a PD-L1 binding (anti-PD-L1) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a CEA binding (anti-CEA) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a CEA binding (anti-CEA) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a CEA binding (anti-CEA) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a CEA binding (anti-CEA) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a CEA binding (anti-CEA) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a CEA binding (anti-CEA) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a CEA binding (anti-CEA) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a CD73 binding (anti-CD73) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a CD73 binding (anti-CD73) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a CD73 binding (anti-CD73) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a CD73 binding (anti-CD73) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a CD73 binding (anti-CD73) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a CD73 binding (anti-CD73) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a CD73 binding (anti-CD73) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a TROP-2 binding (anti-TROP-2) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a TROP-2 binding (anti-TROP-2) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a TROP-2 binding (anti-TROP-2) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a TROP-2 binding (anti-TROP-2) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a TROP-2 binding (anti-TROP-2) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a TROP-2 binding (anti-TROP-2) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a TROP-2 binding (anti-TROP-2) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises an EPCAM binding (anti-EPCAM) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises an EPCAM binding (anti-EPCAM) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises an EPCAM binding (anti-EPCAM) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises an EPCAM binding (anti-EPCAM) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises an EPCAM binding (anti-EPCAM) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises an EPCAM binding (anti-EPCAM) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises an EPCAM binding (anti-EPCAM) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a tissue factor binding (anti-TF) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a tissue factor binding (anti-TF) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a tissue factor binding (anti-TF) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a tissue factor binding (anti-TF) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a tissue factor binding (anti-TF) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a tissue factor binding (anti-TF) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a tissue factor binding (anti-TF) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises an LFA-1 binding (anti-LFA-1) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises an LFA-1 binding (anti-LFA-1) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises an LFA-1 binding (anti-LFA-1) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises an LFA-1 binding (anti-LFA-1) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises an LFA-1 binding (anti-LFA-1) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises an LFA-1 binding (anti-LFA-1) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises an LFA-1 binding (anti-LFA-1) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a FAP binding (anti-FAP) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a FAP binding (anti-FAP) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a FAP binding (anti-FAP) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a FAP binding (anti-FAP) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a FAP binding (anti-FAP) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a FAP binding (anti-FAP) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a FAP binding (anti-FAP) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a VISTA binding (anti-ISTA) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a VISTA binding (anti-VISTA) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a VISTA binding (anti-VISTA) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a VISTA binding (anti-VISTA) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a VISTA binding (anti-VISTA) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a VISTA binding (anti-VISTA) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a VISTA binding (anti-VISTA) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises an LRRC15 binding (anti-LRRC15) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises an LRRC15 binding (anti-LRRC15) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises an LRRC15 binding (anti-LRRC15) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises an LRRC15 binding (anti-VISTA) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises an LRRC15 binding (anti-LRRC15) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises an LRRC15 binding (anti-RRC15) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a LRRC15 binding (anti-LRRC15) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a DLL3 binding (anti-DLL3) domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a DLL3 binding (anti-DLL3) domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a DLL3 binding (anti-DLL3) domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a DLL3 binding (anti-DLL3) domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a DLL3 binding (anti-DLL3) domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a DLL3 binding (anti-DLL3) domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a DLL3 binding (anti-DLL3) domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a PD-L1 binding (anti-PD-L1) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a PD-L1 binding (anti-PD-L1) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a PD-L1 binding (anti-PD-L1) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a PD-L1 binding (anti-PD-L1) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a PD-L1 binding (anti-PD-L1) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a PD-L1 binding (anti-PD-L1) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a PD-L1 binding (anti-PD-L1) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a CEA binding (anti-CEA) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a CEA binding (anti-CEA) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a CEA binding (anti-CEA) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a CEA binding (anti-CEA) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a CEA binding (anti-CEA) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a CEA binding (anti-CEA) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a CEA binding (anti-CEA) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a CD73 binding (anti-CD73) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a CD73 binding (anti-CD73) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a CD73 binding (anti-CD73) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a CD73 binding (anti-CD73) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a CD73 binding (anti-CD73) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a CD73 binding (anti-CD73) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a CD73 binding (anti-CD73) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a TROP-2 binding (anti-TROP-2) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a TROP-2 binding (anti-TROP-2) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a TROP-2 binding (anti-TROP-2) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a TROP-2 binding (anti-TROP-2) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a TROP-2 binding (anti-TROP-2) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a TROP-2 binding (anti-TROP-2) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a TROP-2 binding (anti-TROP-2) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises an EPCAM binding (anti-EPCAM) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises an EPCAM binding (anti-EPCAM) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises an EPCAM binding (anti-EPCAM) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises an EPCAM binding (anti-EPCAM) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises an EPCAM binding (anti-EPCAM) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises an EPCAM binding (anti-EPCAM) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises an EPCAM binding (anti-EPCAM) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a tissue factor binding (anti-TF) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a tissue factor binding (anti-TF) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a tissue factor binding (anti-TF) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a tissue factor binding (anti-TF) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a tissue factor binding (anti-TF) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a tissue factor binding (anti-TF) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a tissue factor binding (anti-TF) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises an LFA-1 binding (anti-LFA-1) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises an LFA-1 binding (anti-LFA-1) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises an LFA-1 binding (anti-LFA-1) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises an LFA-1 binding (anti-LFA-1) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises an LFA-1 binding (anti-LFA-1) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises an LFA-1 binding (anti-LFA-1) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises an LFA-1 binding (anti-LFA-1) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a FAP binding (anti-FAP) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a FAP binding (anti-FAP) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a FAP binding (anti-FAP) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a FAP binding (anti-FAP) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a FAP binding (anti-FAP) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a FAP binding (anti-FAP) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a FAP binding (anti-FAP) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a VISTA binding (anti-VISTA) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a VISTA binding (anti-VISTA) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a VISTA binding (anti-VISTA) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a VISTA binding (anti-VISTA) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a VISTA binding (anti-VISTA) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a VISTA binding (anti-VISTA) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a VISTA binding (anti-VISTA) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises an LRRC15 binding (anti-LRRC15) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises an LRRC15 binding (anti-LRRC15) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises an LRRC15 binding (anti-LRRC15) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises an LRRC15 binding (anti-VISTA) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises an LRRC15 binding (anti-LRRC15) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises an LRRC15 binding (anti-LRRC15) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a LRRC15 binding (anti-LRRC15) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises a protein sequence that comprises a DLL3 binding (anti-DLL3) domain, a hinge domain, a transmembrane domain, and a CD28 intracellular domain (for example, the domain of SEQ ID NO:572).

In some embodiments, the present invention includes a TIL, MIL, or PBL that expresses a CCR that comprises a protein sequence that comprises a DLL3 binding (anti-DLL3) domain, a hinge domain, a transmembrane domain, and a CD134 (OX40) intracellular domain (for example, the domain of SEQ ID NO:573).

In some embodiments, the present invention includes a TIL, MIL, or PBL that expresses a CCR that comprises a protein sequence that comprises a DLL3 binding (anti-DLL3) domain, a hinge domain, a transmembrane domain, and a CD134 (ICOS) intracellular domain (for example, the domain of SEQ ID NO:574).

In some embodiments, the present invention includes a TIL, MIL, or PBL that expresses a CCR that comprises a protein sequence that comprises a DLL3 binding (anti-DLL3) domain, a hinge domain, a transmembrane domain, and a CD137 (4-1BB) intracellular domain (for example, the domain of SEQ ID NO:575).

In some embodiments, the present invention includes a TIL, MIL, or PBL that expresses a CCR that comprises a protein sequence that comprises a DLL3 binding (anti-DLL3) domain, a hinge domain, a transmembrane domain, and a CD27 intracellular domain (for example, the domain of SEQ ID NO:576).

In some embodiments, the present invention includes a TIL, MIL, or PBL that expresses a CCR that comprises a protein sequence that comprises a DLL3 binding (anti-DLL3) domain, a hinge domain, a transmembrane domain, and an IL-2Rβ intracellular domain (for example, the domain of SEQ ID NO:578).

In some embodiments, the present invention includes a TIL, MIL, or PBL that expresses a CCR that comprises a protein sequence that comprises a DLL3 binding (anti-DLL3) domain, a hinge domain, a transmembrane domain, and an IL-18R1 intracellular domain (for example, the domain of SEQ ID NO:580).

In some embodiments, the present invention includes a TIL, MIL, or PBL that expresses a CCR that comprises protein sequence that comprises an anti-PD-L1 scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-CEA scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-CD73 scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-TROP-2 scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-EPCAM scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-tissue factor scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-LFA-1 scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-FAP scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-VISTA scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-LRRC15 scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-DLL3 scFv binding domain, an optional CD8α hinge domain, a CD28 transmembrane domain, and a CD28 intracellular domain.

In some embodiments, the present invention includes a TIL, MIL, or PBL that expresses a CCR that comprises protein sequence that comprises an anti-PD-L1 scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-CEA scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-CD73 scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-TROP-2 scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-EPCAM scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-tissue factor scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-LFA-1 scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-FAP scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-VISTA scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-LRRC15 scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-DLL3 scFv binding domain, an optional CD8α hinge domain, a CD28 transmembrane domain, and an IL-18R1 intracellular domain.

In some embodiments, the present invention includes a TIL, MIL, or PBL that expresses a CCR that comprises protein sequence that comprises an anti-PD-L1 scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-CEA scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-CD73 scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-TROP-2 scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-EPCAM scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-tissue factor scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-LFA-1 scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-FAP scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-VISTA scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-LRRC15 scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises protein sequence that comprises an anti-DLL3 scFv binding domain, an optional CD8α hinge domain, a CD28 transmembrane domain, and an IL-2Rβ intracellular domain.

In some embodiments, the present invention includes a TIL, MIL, or PBL that expresses a CCR that comprises a protein sequence comprises an anti-PD-L1 scFv binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-CEA scFv binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-LRRC15 binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-TROP-2 binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-EPCAM binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-tissue factor binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-LFA-1 binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-FAP binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence that comprises an anti-VISTA scFv binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-LRRC15 scFv binding domain, a CD28 transmembrane domain, a CD28 intracellular domain, and an IL-18R1 intracellular domain.

In some embodiments, the present invention includes a TIL, MIL, or PBL that expresses a CCR that comprises a protein sequence comprises an anti-DLL3 binding domain, a CD28 transmembrane domain, a CD28 intracellular domain, and an IL-18R1 intracellular domain.

In some embodiments, the present invention includes a TIL, MIL, or PBL that expresses a CCR that comprises protein sequence that comprises an anti-PD-L1 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-CEA scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-CD73 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-TROP-2 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-EPCAM scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-tissue factor scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-LFA-1 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-FAP scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-VISTA scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-LRRC15 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-DLL3 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-PD-L1 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-CEA scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-CD73 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-TROP-2 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-EPCAM scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-tissue factor scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-LFA-1 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-FAP scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-VISTA scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-LRRC15 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-DLL3 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-PD-L1 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-CEA scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-CD73 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-TROP-2 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-EPCAM scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-tissue factor scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-LFA-1 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-FAP scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-VISTA scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-LRRC15 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-DLL3 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-PD-L1 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-CEA scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-CD73 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-TROP-2 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-EPCAM scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-tissue factor scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-LFA-1 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-FAP scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-VISTA scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-LRRC15 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-DLL3 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-PD-L1 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-CEA scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-CD73 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-TROP-2 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-EPCAM scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-tissue factor scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-LFA-1 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-FAP scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-VISTA scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-LRRC15 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-DLL3 scFv binding domain, a CD8α hinge domain, a CD8α transmembrane domain, and an IL-2Rβ intracellular domain.

In some embodiments, the present invention includes a TIL, MIL, or PBL that expresses a CCR that comprises protein sequence that comprises an anti-PD-L1 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-CEA scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-CD73 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-TROP-2 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-EPCAM scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-tissue factor scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-LFA-1 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-FAP scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-VISTA scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-LRRC15 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-B7-H3 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-DLL3 scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-FAS scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-TGFβRII scFv binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises a PD1 binding domain, a IgG4 hinge domain, a IgG4 transmembrane domain, and an IL-2Rβ intracellular domain.

In some embodiments, the present invention includes a TIL, MIL, or PBL that expresses a CCR that comprises protein sequence that comprises an anti-PD-L1 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-CEA scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-CD73 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-TROP-2 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-EPCAM scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-tissue factor scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-LFA-1 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-FAP scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-VISTA scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-LRRC15 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-B7-H3 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-DLL3 scFv binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-FAS binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-TGFβRII binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises a PD1 binding domain, a CD8α hinge domain, a CD8α transmembrane domain, and an IL-18R1 intracellular domain.

In some embodiments, the present invention includes a TIL, MIL, or PBL that expresses a CCR that comprises protein sequence that comprises an anti-PD-L1 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-CEA scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-CD73 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-TROP-2 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-EPCAM scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-tissue factor scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-LFA-1 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-FAP scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-VISTA scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-LRRC15 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-B7-H3 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-DLL3 scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-FAS scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-TGFβRII scFv binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises a PD-1 binding domain, a CD8α hinge domain, a CD8α transmembrane domain, and a CD27 intracellular domain.

In some embodiments, the present invention includes a TIL, MIL, or PBL that expresses a CCR that comprises protein sequence that comprises an anti-PD-L1 scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-CEA scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-CD73 scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-TROP-2 scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-EPCAM scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-tissue factor scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-LFA-1 scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-FAP scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-VISTA scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-LRRC15 scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-B7-H3 scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-DLL3 scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-FAS scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises an anti-TGFβRII scFv binding domain, a CD8α hinge domain, a CD8α transmembrane domain, and a 4-1BB intracellular domain.

In some embodiments, the present invention includes a TIL, MIL, or PBL that expresses a CCR that comprises protein sequence that comprises a PD-1 binding domain, a CD8α hinge domain, a CD8α transmembrane domain, and a 4-1BB intracellular domain.

In some embodiments, the present invention includes a TIL, MIL, or PBL that expresses a CCR that comprises a protein comprising (anti-TROP-2-V_{L})-(linker)-(anti-TROP-2-V_{H})-(CD8α hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(CD8α hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(CD8α hinge and transmembrane)-(CD27), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(CD8α hinge and transmembrane)-(CD28), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(CD8α hinge and transmembrane)-(CD137), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(CD8α hinge and transmembrane)-(CD134), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(CD8α hinge and transmembrane)-(CD278), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(IgG4 hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(IgG4 hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(IgG4 hinge and transmembrane)-(CD27), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(IgG4 hinge and transmembrane)-(CD28), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(IgG4 hinge and transmembrane)-(CD137), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(IgG4 hinge and transmembrane)-(CD134), wherein each domain denoted by parenthesis is operatively linked.

In some embodiments, the present invention includes a TIL, MIL, or PBL that expresses a CCR that comprises a protein comprising (anti-TROP-2-V_{L})-(linker)-(anti-TROP-2-V_{H})-(IgG4 hinge and transmembrane)-(CD278), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(CD27), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(CD28), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(CD137), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(CD134), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(CD8α hinge and transmembrane)-(CD278), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(IgG4 hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(IgG4 hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(IgG4 hinge and transmembrane)-(CD27), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(IgG4 hinge and transmembrane)-(CD28), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(IgG4 hinge and transmembrane)-(CD137), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(IgG4 hinge and transmembrane)-(CD134), wherein each domain denoted by parenthesis is operatively linked.

In some embodiments, the present invention includes a TIL, MIL, or PBL that expresses a CCR that comprises a protein comprising (anti-FAP-V_{L})-(linker)-(anti-FAP-V_{H})-(IgG4 hinge and transmembrane)-(CD278), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(CD27), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(CD28), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(CD137), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(CD134), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(CD8α hinge and transmembrane)-(CD278), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(IgG4 hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(IgG4 hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(IgG4 hinge and transmembrane)-(CD27), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(IgG4 hinge and transmembrane)-(CD28), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(IgG4 hinge and transmembrane)-(CD137), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(IgG4 hinge and transmembrane)-(CD134), wherein each domain denoted by parenthesis is operatively linked.

In some embodiments, the present invention includes a TIL, MIL, or PBL that expresses a CCR that comprises a protein comprising (anti-DLL3-V_{L})-(linker)-(anti-DLL3-V_{H})-(IgG4 hinge and transmembrane)-(CD278), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(CD27), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(CD28), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(CD137), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(CD134), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(CD278), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(IgG4 hinge and transmembrane)-(IL-2Rβ), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(IgG4 hinge and transmembrane)-(IL-18R1), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(IgG4 hinge and transmembrane)-(CD27), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(IgG4 hinge and transmembrane)-(CD28), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(IgG4 hinge and transmembrane)-(CD137), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(IgG4 hinge and transmembrane)-(CD134), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(IgG4 hinge and transmembrane)-(CD278), wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(CD27), using the 38A1 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8α hinge and transmembrane)-(CD27), using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(hinge and transmembrane)-(4-1BB intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (CD8 signal peptide)-(anti-PD-L1-V_{L})-(linker)-(anti-PD-L1-V_{H})-(hinge and transmembrane)-(4-1BB intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8 hinge and transmembrane)-(4-1BB intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (CD8 signal peptide)-(anti-PD-L1-V_{L})-(linker)-(anti-PD-L1-V_{H})-(CD8 hinge and transmembrane)-(4-1BB intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(hinge and transmembrane)-(LTBR intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (CD8 signal peptide)-(anti-PD-LT-V_{L})-(linker)-(anti-PD-L1-V_{H})-(hinge and transmembrane)-(LTBR intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(CD8 hinge and transmembrane)-(LTBR intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (CD8 signal peptide)-(anti-PD-L1-V_{L})-(linker)-(anti-PD-L1-V_{H})-(CD8 hinge and transmembrane)-(LTBR intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (anti-PD-L1-V_{L})-(linker)-(anti-PD-L1-V_{H})-(hinge and transmembrane)-(4-1BB intracellular domain)-(LTBR intracellular domain), using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (CD8 signal peptide)-(anti-PD-Lt-V_{L})-(linker)-(anti-PD-L1-V_{H})-(hinge and transmembrane)-(LTBR intracellular domain)-(4-1BB intracellular domain), optionally using the 19H9 anti-PD-L1 V_{H} and V_{L} domains described herein, wherein each domain denoted by parenthesis is operatively linked.

In some embodiments, the present invention includes a TIL, MIL, or PBL that expresses a CCR that comprises a protein comprising (PD-1 extracellular domain)-(12 amino acids of CD28 extracellular domain)-(CD28 transmembrane domain)-(CD28 intracellular domain), wherein each domain denoted by parenthesis is operatively linked.

In some embodiments, the present invention includes a TIL, MIL, or PBL that expresses a CCR that comprises a protein comprising (PD-1 extracellular domain)-(12 amino acids of CD28 extracellular domain)-(CD28 transmembrane domain)-(CD28 intracellular domain), wherein each domain denoted by parenthesis is operatively linked.

In some embodiments, the present invention includes a TIL, MIL, or PBL that expresses a CCR that comprises a protein comprising (PD-1 extracellular domain)-(12 amino acids of 4-1BB extracellular domain)-(4-1BB transmembrane domain)-(4-1BB intracellular domain), wherein each domain denoted by parenthesis is operatively linked.

In some embodiments, the present invention includes a TIL, MIL, or PBL that expresses a CCR that comprises a protein comprising (TGFβRII extracellular domain)-(12 amino acids of CD28 extracellular domain)-(CD28 transmembrane domain)-(CD28 intracellular domain), wherein each domain denoted by parenthesis is operatively linked.

In some embodiments, the present invention includes a TIL, MIL, or PBL that expresses a CCR that comprises a protein comprising (TGFβRII extracellular domain)-(12 amino acids of 4-1BB extracellular domain)-(4-1BB transmembrane domain)-(4-1BB intracellular domain), wherein each domain denoted by parenthesis is operatively linked.

In some embodiments, the present invention includes a TIL, MIL, or PBL that expresses a CCR that comprises a protein comprising (FAS extracellular domain)-(FAS transmembrane domain)-(7 amino acids of FAS intracellular domain)-(4-1BB intracellular domain), wherein each domain denoted by parenthesis is operatively linked.

In some embodiments, the present invention includes a TIL, MIL, or PBL that expresses a CCR that comprises a protein comprising (IgE signal peptide)-(PD-1 extracellular domain)-(12 amino acids of CD28 extracellular domain)-(CD28 transmembrane domain)-(CD28 intracellular domain), wherein each domain denoted by parenthesis is operatively linked.

In some embodiments, the present invention includes a TIL, MIL, or PBL that expresses a CCR that comprises a protein comprising (IgE signal peptide)-(PD-1 extracellular domain)-(12 amino acids of 4-1BB extracellular domain)-(4-1BB transmembrane domain)-(4-1BB intracellular domain), wherein each domain denoted by parenthesis is operatively linked.

In some embodiments, the present invention includes a TIL, MIL, or PBL that expresses a CCR that comprises a protein comprising (IgE signal peptide)-(TGFβRII extracellular domain)-(12 amino acids of CD28 extracellular domain)-(CD28 transmembrane domain)-(CD28 intracellular domain), wherein each domain denoted by parenthesis is operatively linked.

In some embodiments, the present invention includes a TIL, MIL, or PBL that expresses a CCR that comprises a protein comprising (IgE signal peptide)-(TGFβRII extracellular domain)-(12 amino acids of 4-1BB extracellular domain)-(4-1BB transmembrane domain)-(4-1BB intracellular domain), wherein each domain denoted by parenthesis is operatively linked.

In some embodiments, the present invention includes a TIL, MIL, or PBL that expresses a CCR that comprises a protein comprising (IgE signal peptide)-(FAS extracellular domain)-(FAS transmembrane domain)-(7 amino acids of FAS intracellular domain)-(4-1BB intracellular domain), wherein each domain denoted by parenthesis is operatively linked.

In some embodiments, the present invention includes a TIL, MIL, or PBL that expresses a biepitope CCR construct.

Nucleotide sequences of vectors encoding exemplary CCRs of the present invention are provided in Table 63. In an embodiment, a nucleotide sequence in Table 63 is codon-optimized to improve protein expression. In an embodiment, a nucleotide sequence in Table 63 is further modified to include additional linker domains, as described elsewhere herein. In an embodiment, a nucleotide sequence in Table 63 is used in a lentiviral expression system. In an embodiment, a nucleotide sequence in Table 63 is used in a lentiviral expression system using additional plasmids.

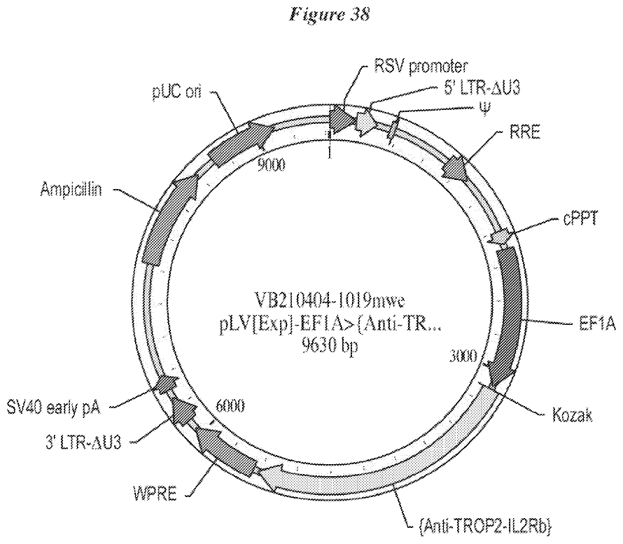

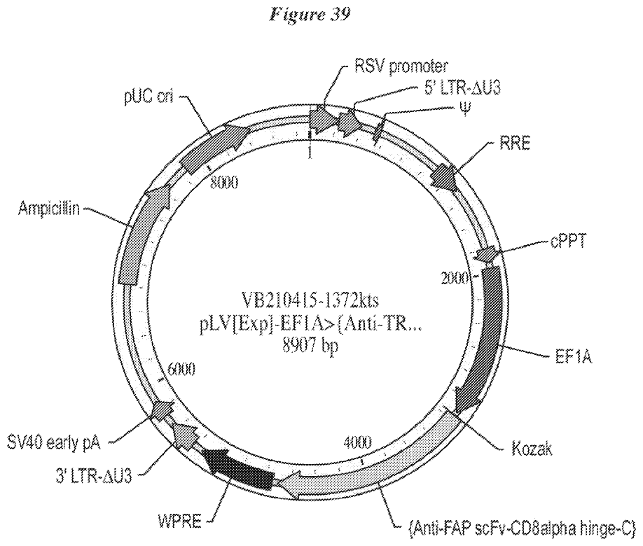

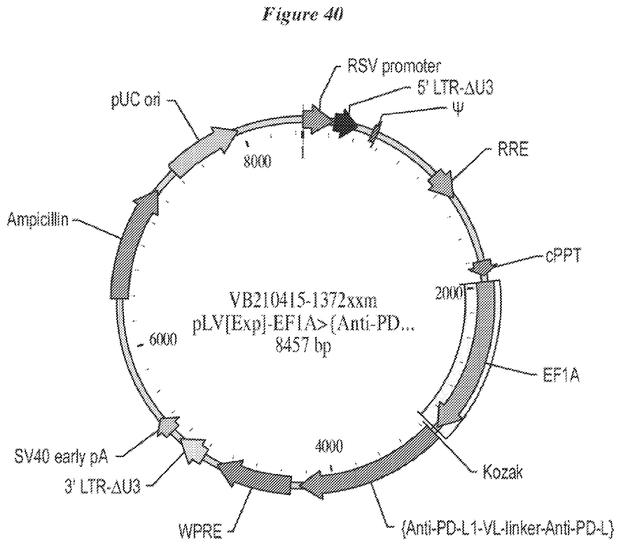

Exemplary vector designs for the vectors provided in Table 63 are provided in FIGS. 38 to 40. In an embodiment, a CCR encoded by the vector shown in FIG. 38 is used to genetically modify a TIL product of the present invention as described herein. In an embodiment, a CCR encoded by the vector shown in FIG. 39 is used to genetically modify a TIL product of the present invention as described herein. In an embodiment, a CCR encoded by the vector shown in FIG. 40 is used to genetically modify a TIL product of the present invention as described herein.

TABLE 63
Nucleotide sequences of exemplary vectors for expression of CCRs.

| Identifier | Sequence (One-Letter Nucleotide Symbols) | |
|---|---|---|
| SEQ ID NO: 618 | AATGTAGTCT TATGCAATAC TCTTGTAGTC TTGCAACATG GTAACGATGA GTTAGCAACA | 60 |
| Anti-TROP2-V_{L}- | TGCCTTACAA GGAGAGAAAA AGCACCGTGC ATGCCGATTG GTGGAAGTAA GGTGGTACGA | 120 |
| linker- | TCGTGCCTTA TTAGGAAGGC AACAGACGGG TCTGACATGG ATTGGACGAA CCACTGAATT | 180 |
| Anti-TROP2-V_{H}- | GCCGCATTGC AGAGATATTG TATTTAAGTG CCTAGCTCGA TACATAAACG GGTCTCTCTG | 240 |
| IgG4 (hinge and | GTTAGACCAG ATCTGAGCCT GGGAGCTCTC TGGCTAACTA GGGAACCCAC TGCTTAAGCC | 300 |
| transmembrane)- | TCAATAAAGC TTGCCTTGAG TGCTTCAAGT AGTGTGTGCC CGTCTGTTGT GTGACTCTGG | 360 |
| IL2Rβ | TAACTAGAGA TCCCTCAGAC CCTTTTAGTC AGTGTGGAAA ATCTCTAGCA GTGGCGCCCG | 420 |
| | AACAGGGACT TGAAAGCGAA AGGGAAACCA GAGGAGCTCT CTCGACGCAG GACTCGGCTT | 480 |
| | GCTGAAGCGC GCACGGCAAG AGGCGAGGGG CGGCGACTGG TGAGTACGCC AAAAATTTTG | 540 |
| | ACTAGCGGAG GCTAGAAGGA GAGAGATGGG TGCGAGAGCG TCAGTATTAA GCGGGGGAGA | 600 |
| | ATTAGATCGC GATGGGAAAA AATTCGGTTA AGGCCAGGGG GAAAGAAAAA ATATAAATTA | 660 |
| | AAACATATAG TATGGGCAAG CAGGGAGCTA GAACGATTCG CAGTTAATCC TGGCCTGTTA | 720 |
| | GAAACATCAG AAGGCTGTAG ACAAATACTG GGACAGCTAC AACCATCCCT TCAGACAGGA | 780 |
| | TCAGAAGAAC TTAGATCATT ATATAATACA GTAGCAACCC TCTATTGTGT GCATCAAAGG | 840 |
| | ATAGAGATAA AAGACACCAA GGAAGCTTTA GACAAGATAG AGGAAGAGCA AAACAAAAGT | 900 |
| | AAGACCACCG CACAGCAAGC GGCCGCTGAT CTTCAGACCT GGAGGAGGAG ATATGAGGGA | 960 |
| | CAATTGGAGA AGTGAATTAT ATAAATATAA AGTAGTAAAA ATTGAACCAT TAGGAGTAGC | 1020 |
| | ACCCACCAAG GCAAAGAGAA GAGTGGTGCA GAGAGAAAAA AGAGCAGTGG GAATAGGAGC | 1080 |
| | TTTGTTCCTT GGGTTCTTGG GAGCAGCAGG AAGCACTATG GGCGCAGCGT CAATGACGCT | 1140 |
| | GACGGTACAG GCCAGACAAT TATTGTCTGG TATAGTGCAG CAGCAGAACA ATTTGCTGAG | 1200 |
| | GGCTATTGAG GCGCAACAGC ATCTGTTGCA ACTCACAGTC TGGGGCATCA AGCAGCTCCA | 1260 |
| | GGCAAGAATC CTGGCTGTGG AAAGATACCT AAAGGATCAA CAGCTCCTGG GGATTTGGGG | 1320 |
| | TTGCTCTGGA AAACTCATTT GCACCACTGC TGTGCCTTGG AATGCTAGTT GGAGTAATAA | 1380 |
| | ATCTCTGGAA CAGASTTGGA ATCACACGAC CTGGATGGAG TGGGACAGAG AAATTAACAA | 1440 |
| | TTACACAAGC TTAATACACT CCTTAATTGA AGAATCGCAA AACCAGCAAG AAAAGAATGA | 1500 |
| | ACAAGAATTA TTGGAATTAG ATAAATGGGC AAGTTTGTGG AATTGGTTTA ACATAACAAA | 1560 |
| | TTGGCTGTGG TATATAAAAT TATTCATAAT GATAGTAGGA GGCTTGGTAG GTTTAAGAAT | 1620 |
| | AGTTTTTGCT GTACTTTCTA TAGTGAATAG AGTTAGGCAG GGATATTCAC CATTATCGTT | 1680 |
| | TCAGACCCAC CTCCCAACCC CGAGGGGACC CGACAGGCCC GAAGGAATAG AAGAAGAAGG | 1740 |
| | TGGAGAGAGA GACAGAGACA GATCCATTCG ATTAGTGAAC GGATCTCGAC GGTATCGCTA | 1800 |
| | GCTTTTAAAA GAAAAGGGGG GATTGGGGGG TACAGTGCAG GGGAAAGAAT AGTAGACATA | 1860 |
| | ATAGCAACAG ACATACAAAC TAAAGAATTA CAAAAACAAA TTACAAAAAT TCAAAATTTT | 1920 |
| | ACTAGTGATT ATCGGATCAA CTTTGTATAG AAAAGTTGGG CTCCGGTGCC CGTCAGTGGG | 1980 |
| | CAGAGCGCAC ATCGCCCACA GTCCCCGAGA AGTTGGGGGG AGGGGTCGGC AATTGAACCG | 2040 |
| | GTGCCTAGAG AAGGTGGCGC GGGGTAAACT GGGAAAGTGA TGTCGTGTAC TGGCTCCGCC | 2100 |
| | TTTTTCCCGA GGGTGGGGGA GAACCGTATA TAAGTGCAGT AGTCGCCGTG AACGTTCTTT | 2160 |
| | TTCGCAACGG GTTTGCCGCC AGAACACAGG TAAGTGCCGT GTGTGGTTCC CGCGGGCCTG | 2220 |
| | GCCTCTTTAC GGGTTATGGC CCTTGCGTGC CTTGAATTAC TTCCACCTGG CTGCAGTACG | 2280 |
| | TGATTCTTGA TCCCGAGCTT CGGGTTGGAA GTGGGTGGGA GAGTTCGAGG CCTTGCGCTT | 2340 |
| | AAGGAGCCCC TTCGCCTCGT GCTTGAGTTG AGGCCTGGCC TGGGCGCTGG GGCCGCCGCG | 2400 |
| | TGCGAATCTG GTGGCACCTT CGCGCCTGTC TCGCTGCTTT CGATAAGTCT CTAGCCATTT | 2460 |
| | AAAATTTTTG ATGACCTGCT GCGACGCTTT TTTTCTGGCA AGATAGTCTT GTAAATGCGG | 2520 |
| | GCCAAGATCT GCACACTGGT ATTTCGGTTT TTGGGGCCGC GGGCGGCGAC GGGGCCCGTG | 2580 |
| | CGTCCCAGCG CACATGTTCG GCGAGGCGGG GCCTGCGAGC GCGGCCACCG AGAATCGGAC | 2640 |
| | GGGGGTAGTC TCAAGCTGGC CGGCCTGCTC TGGTGCCTGG TCTCGCGCCG CCGTGTATCG | 2700 |
| | CCCCGCCCTG GGCGGCAAGG CTGGCCCGGT CGGCACCAGT TGCGTGAGCG GAAAGATGGC | 2760 |
| | CGCTTCCCGG CCCTGCTGCA GGGAGCTCAA AATGGAGGAC GCGGCGCTCG GGAGAGCGGG | 2820 |
| | CGGGTGAGTC ACCCACACAA AGGAAAAGGG CCTTTCCGTC CTCAGCCGTC GCTTCATGTG | 2880 |
| | ACTCCACGGA GTACCGGGCG CCGTCCAGGC ACCTCGATTA GTTCTCGAGC TTTTGGAGTA | 2940 |
| | CGTCGTCTTT AGGTEGGGGG GAGGGGTTTT ATGCGATGGA GTTTCCCCAC ACTGAGTGGG | 3000 |
| | TGGAGACTGA AGTTAGGCCA GCTTGGCACT TGATGTAATT CTCCTTGGAA TTTGCCCTTT | 3060 |
| | TTGAGTTTGG ATCTTGGTTC ATTCTCAAGC CTCAGACAGT GGTTCAAAGT TTTTTTCTTC | 3120 |
| | CATTTCAGGT GTCGTGACAA GTTTGTACAA AAAAGCAGGC TGCCACCATG GAGATCGTGC | 3180 |
| | TGACCCAAAG TCCAGCCACC CTTTCCCTGT CTCCAGGCGA ACGCGCAACC CTGAGCTGCC | 3240 |
| | GCGCTTCTCA GACCATTGGT ACCTCCATTC ATTGGTATCA GCAGAAGCCC GGCCAAGCCC | 3300 |
| | CGCGTCTGCT GATCTATTAC GCCTCAGAAA GTATTTCAGG CATCCCCGCT CGCTTCTCCG | 3360 |
| | GCTCCGGCAG CGGAACCGAC TTCACACTTA CAATCTCTAG TTTGGAGCCA GAAGACTTCG | 3420 |
| | CCGTTTACTA CTGTCAGCAG TCTAACAGCT GGCCATTTAC CTTTGGCCAG GGCACGAAGC | 3480 |
| | TGGAAATCAA GGGCGGCGGC GGCAGCGGCG GCGGCGGCAG CGGCGGCGGC GGCAGCCAGG | 3540 |
| | TGCAGTTGGT TCAGAGCGGC GCGGAAGTCA AGAAACCCGG CGCCTCCGTG AAAGTGAGCT | 3600 |
| | GCAAAGCGAG CGGCTACACC TTCACCAGTT ATTGGATTAA CTGGGTGCGC CAGGCCCCAG | 3660 |
| | GCCAGGGGCT GGAGTGGATG GGAAACATCT ACCCATCTGA CTCTTACAGC AACTATAATC | 3720 |
| | AGAAATTTAA GGATCGCGTA ACAATGACCC GTGACACCAG CACCAGCACT GTTTACATGG | 3780 |
| | AGCTGAGTTC TCTGCGTTCT GAAGATACCG CCGTGTACTA CTGCGCACGC GGTTCCAGTT | 3840 |
| | TCGATTACTG GGGCCAGGGG ACCCTGGTCA CCGTCTCCTC AGAGAGCAAG TACGGCCCTC | 3900 |
| | CCTGCCCCCC TTGCCCTGCC CCCGAGTTCC TGGGCGGACC CAGCGTGTTC CTGTTCCCCC | 3960 |
| | CCAAGCCCAA GGACACCCTG ATGATCAGCC GGACCCCCGA GGTGACCTGT GTGGTGGTGG | 4020 |
| | ACGTGTCCCA GGAGGACCCC GAGGTCCAGT TCAACTGGTA CGTGGACGGC GTGGAGGTGC | 4080 |
| | ACAACGCCAA GACCAAGCCC CGGGAGGAGC AGTTCAATAG CACCTACCGG GTGGTGTCCG | 4140 |
| | TGCTGACCGT GCTGCACCAG GACTGGCTGA ACGGCAAGGA ATACAAGTGT AAGGTGTCCA | 4200 |
| | ACAAGGGCCT GCCCAGCAGC ATCGAGAAAA CCATCAGCAA GGCCAAGGGC CAGCCTCGGG | 4260 |
| | AGCCCCAGGT GTACACCCTG CCCCCTAGCC AAGAGGAGAT GACCAAGAAC CAGGTGTCCC | 4320 |
| | TGACCTGCCT GGTGAAGGGC TTCTACCCCA GCGACATCGC CGTGGAGTGG GAGAGCAACG | 4380 |
| | GCCAGCCCGA GAACAACTAC AAGACCACCC CCCCTGTGCT GGACAGCGAC GGCAGCTTCT | 4440 |
| | TCCTGTACAG CCGGCTGACC GTGGACAAGA GCCGGTGGCA GGAGGGCAAC GTCTTTAGCT | 4500 |
| | GCTCCGTGAT GCACGAGGCC CTGCACAACC ACTACACCCA GAAGAGCCTG AGCCTGTCCC | 4560 |
| | TGGGCAAGAT GAACTGCAGG AACACCGGGC CATGGCTGAA GAAGGTCCTG AAGTGTAACA | 4620 |
| | CCCCAGACCC CTCGAAGTTC TTTTCCCAGC TGAGCTCAGA GCATGGAGGA GACGTCCAGA | 4680 |
| | AGTGGCTCTC TTCGCCCTTC CCCTCATCGT CCTTCAGCCC TGGCGGCCTG GCACCTGAGA | 4740 |
| | TCTCGCCACT AGAAGTGCTG GAGAGGGACA AGGTGACGCA GCTGCTCCTG CAGCAGGACA | 4800 |
| | AGGTGCCTGA GCCCGCATCC TTAAGCAGCA ACCACTCGCT GACCAGCTGC TTCACCAACC | 4860 |
| | AGGGTTACTT CTTCTTCCAC CTCCCGGATG CCTTGGAGAT AGAGGCCTGC CAGGTGTACT | 4920 |
| | TTACTTACGA CCCCTACTCA GAGGAAGACC CTGATGAGGG TGTGGCCGGG GCACCCACAG | 4980 |
| | GGTCTTCCCC CCAACCCCTG CAGCCTCTGT CAGGGGAGGA CGACGCCTAC TGCACCTTCC | 5040 |
| | CCTCCAGGGA TGACCTGCTG CTCTTCTCCC CCAGTCTCCT CGGTGGCCCC AGCCCCCCAA | 5100 |
| | GCACTGCCCC TGGGGGCAGT GGGGCCGGTG AAGAGAGGAT GCCCCCTTCT TTGCAAGAAA | 5160 |
| | GAGTCCCCAG AGACTGGGAC CCCCAGCCCC TGGGGCCTCC CACCCCAGGA GTCCCAGACC | 5220 |
| | TGGTGGATTT TCAGCCACCC CCTGAGCTGG TGCTGCGAGA GGCTGGGGAG GAGGTCCCTG | 5280 |
| | ACGCTGGCCC CAGGGAGGGA GTCAGTTTCC CCTGGTCCAG GCCTCCTGGG CAGGGGGAGT | 5340 |
| | TCAGGGCCCT TAATGCTCGC CTGCCCCTGA ACACTGATGC CTACTTGTCC CTCCAAGAAC | 5400 |
| | TCCAGGGTCA GGACCCAACT CACTTGGTGT AAACCCAGCT TTCTTGTACA AAGTGGTGAT | 5460 |
| | AATCGAATTC CGATAATCAA CCTCTGGATT ACAAAATTTG TGAAAGATTG ACTGGTATTC | 5520 |
| | TTAACTATGT TGCTCCTTTT ACGCTATGTG GATACGCTGC TTTAATGCCT TTGTATCATG | 5580 |
| | CTATTGCTTC CCGTATGGCT TTCATTTTCT CCTCCTTGTA TAAATCCTGG TTGCTGTCTC | 5640 |
| | TTTATGAGGA GTTGTGGCCC GTTGTCAGGC AACGTGGCGT GGTGTGCACT GTGTTTGCTG | 5700 |
| | ACGCAACCCC CACTGGTTGG GGCATTGCCA CCACCTGTCA GCTCCTTTCC GGGACTTTCG | 5760 |
| | CTTTCCCCCT CCCTATTGCC ACGGCGGAAC TCATCGCCGC CTGCCTTGCC CGCTGCTGGA | 5820 |
| | CAGGGGCTCG GCTGTTGGGC ACTGACAATT CCGTGGTGTT GTCGGGGAAG CTGACGTCCT | 5880 |
| | TTCCATGGCT GCTCGCCTGT GTTGCCACCT GGATTCTGCG CGGGACGTCC TTCTGCTACG | 5940 |
| | TCCCTTCGGC CCTCAATCCA GCGGACCTTC CTTCCCGCGG CCTGCTGCCG GCTCTGCGGC | 6000 |
| | CTCTTCCGCG TCTTCGCCTT CGCCCTCAGA CGAGTCGGAT CTCCCTTTGG GCCGCCTCCC | 6060 |
| | CGCATCGGGA ATTCCCGCGG TTCGCTTTAA GACCAATGAC TTACAAGGCA GCTGTAGATC | 6120 |
| | TTAGCCACTT TTTAAAAGAA AAGGGGGGAC TGGAAGGGCT AATTCACTCC CAACGAAGAC | 6180 |
| | AAGATCTGCT TTTTGCTTGT ACTGGGTCTC TCTGGTTAGA CCAGATCTGA GCCTGGGAGC | 6240 |
| | TCTCTGGCTA ACTAGGGAAC CCACTGCTTA AGCCTCAATA AAGCTTGCCT TGAGTGCTTC | 6300 |
| | AAGTAGTGTG TGCCCGTCTG TTGTGTGACT CTGGTAACTA GAGATCCCTC AGACCCTTTT | 6360 |
| | AGTCAGTGTG GAAAATCTCT AGCAGTAGTA GTTCATGTCA TCTTATTATT CAGTATTTAT | 6420 |
| | AACTTGCAAA GAAATGAATA TCAGAGAGTG AGAGGAACTT GTTTATTGCA GCTTATAATG | 6480 |
| | GTTACAAATA AAGCAATAGC ATCACAAATT TCACAAATAA AGCATTTTTT TCACTGCATT | 6540 |
| | CTAGTTGTGG TTTGTCCAAA CTCATCAATG TATCTTATCA TGTCTGGCTC TAGCTATCCC | 6600 |
| | GCCCCTAACT CCGCCCATCC CGCCCCTAAC TCCGCCCAGT TCCGCCCATT CTCCGCCCCA | 6660 |
| | TGGCTGACTA ATTTTTTTTA TTTATGCAGA GGCCGAGGCC GCCTCGGCCT CTGAGCTATT | 6720 |
| | CCAGAAGTAG TGAGGAGGCT TTTTTGGAGG CCTAGGGACG TACCCAATTC GCCCTATAGT | 6780 |
| | GAGTCGTATT ACGCGCGCTC ACTGGCCGTC GTTTTACAAC GTCGTGACTG GGAAAACCCT | 6840 |
| | GGCGTTACCC AACTTAATCG CCTTGCAGCA CATCCCCCTT TCGCCAGCTG GCGTAATAGC | 6900 |
| | GAAGAGGCCC GCACCGATCG CCCTTCCCAA CAGTTGCGCA GCCTGAATGG CGAATGGGAC | 6960 |
| | GCGCCCTGTA GCGGCGCATT AAGCGCGGCG GGTGTGGTGG TTACGCGCAG CGTGACCGCT | 7020 |
| | ACACTTGCCA GCGCCCTAGC GCCCGCTCCT TTCGCTTTCT TCCCTTCCTT TCTCGCCACG | 7080 |
| | TTCGCCGGCT TTCCCCGTCA AGCTCTAAAT CGGGGGCTCC CTTTAGGGTT CCGATTTAGT | 7140 |
| | GCTTTACGGC ACCTCGACCC CAAAAAACTT GATTAGGGTG ATGGTTCACG TAGTGGGCCA | 7200 |
| | TCGCCCTGAT AGACGGTTTT TCGCCCTTTG ACGTTGGAGT CCACGTTCTT TAATAGTGGA | 7260 |
| | CTCTTGTTCC AAACEGGAAC AACACTCAAC CCTATCTCGG TCTATTCTTT TGATTTATAA | 7320 |
| | GGGATTTTGC CGATTTCGGC CTATTGGTTA AAAAATGAGC TGATTTAACA AAAATTTAAC | 7380 |
| | GCGAATTTTA ACAAAATATT AACGCTTACA ATTTAGGTGG CACTTTTCGG GGAAATGTGC | 7440 |
| | GCGGAACCCC TATTTGTTTA TTTTTCTAAA TACATTCAAA TATGTATCCG CTCATGAGAC | 7500 |
| | AATAACCCTG ATAAATGCTT CAATAATATT GAAAAAGGAA GAGTATGAGT ATTCAACATT | 7560 |
| | TCCGTGTCGC CCTTATTCCC TTTTTTGCGG CATTTTGCCT TCCTGTTTTT GCTCACCCAG | 7620 |
| | AAACGCTGGT GAAAGTAAAA GATGCTGAAG ATCAGTTGGG TGCACGAGTG GGTTACATCG | 7680 |
| | AACTGGATCT CAACAGCGGT AAGATCCTTG AGAGTTTTCG CCCCGAAGAA CGTTTTCCAA | 7740 |
| | TGATGAGCAC TTTTAAAGTT CTGCTATGTG GCGCGGTATT ATCCCGTATT GACGCCGGGC | 7800 |
| | AAGAGCAACT CGGTCGCCGC ATACACTATT CTCAGAATGA CTTGGTTGAG TACTCACCAG | 7860 |
| | TCACAGAAAA GCATCTTACG GATGGCATGA CAGTAAGAGA ATTATGCAGT GCTGCCATAA | 7920 |
| | CCATGAGTGA TAACACTGCG GCCAACTTAC TTCTGACAAC GATCGGAGGA CCGAAGGAGC | 7980 |
| | TAACCGCTTT TTTGCACAAC ATGGGGGATC ATGTAACTCG CCTTGATCGT TGGGAACCGG | 8040 |
| | AGCTGAATGA AGCCATACCA AACGACGAGC GTGACACCAC GATGCCTGTA GCAATGGCAA | 8100 |
| | CAACGTTGCG CAAACTATTA ACTGGCGAAC TACTTACTCT AGCTTCCCGG CAACAATTAA | 8160 |
| | TAGACTGGAT GGAGGCGGAT AAAGTTGCAG GACCACTTCT GCGCTCGGCC CTTCCGGCTG | 8220 |
| | GCTGGTTTAT TGCTGATAAA TCTGGAGCCG GTGAGCGTGG GTCTCGCGGT ATCATTGCAG | 8280 |
| | CACTGGGGCC AGATGGTAAG CCCTCCCGTA TCGTAGTTAT CTACACGACG GGGAGTCAGG | 8340 |
| | CAACTATGGA TGAACGAAAT AGACAGATCG CTGAGATAGG TGCCTCACTG ATTAAGCATT | 8400 |
| | GGTAACTGTC AGACCAAGTT TACTCATATA TACTTTAGAT TGATTTAAAA CTTCATTTTT | 8460 |
| | AATTTAAAAG GATCTAGGTG AAGATCCTTT TTGATAATCT CATGACCAAA ATCCCTTAAC | 8520 |
| | GTGAGTTTTC GTTCCACTGA GCGTCAGACC CCGTAGAAAA GATCAAAGGA TCTTCTTGAG | 8580 |
| | ATCCTTTTTT TCTGCGCGTA ATCTGCTGCT TGCAAACAAA AAAACCACCG CTACCAGCGG | 8640 |
| | TGGTTTGTTT GCCGGATCAA GAGCTACCAA CTCTTTTTCC GAAGGTAACT GGCTTCAGCA | 8700 |
| | GAGCGCAGAT ACCAAATACT GTTCTTCTAG TGTAGCCGTA GTTAGGCCAC CACTTCAAGA | 8760 |
| | ACTCTGTAGC ACCGCCTACA TACCTCGCTC TGCTAATCCT GTTACCAGTG GCTGCTGCCA | 8820 |
| | GTGGCGATAA GTCGTGTCTT ACCGGGTTGG ACTCAAGACG ATAGTTACCG GATAAGGCGC | 8880 |
| | AGCGGTCGGG CTGAACGGGG GGTTCGTGCA CACAGCCCAG CTTGGAGCGA ACGACCTACA | 8940 |
| | CCGAACTGAG ATACCTACAG CGTGAGCTAT GAGAAAGCGC CACGCTTCCC GAAGAGAGAA | 9000 |
| | AGGCGGACAG GTATCCGGTA AGCGGCAGGG TCGGAACAGG AGAGCGCACG AGGGAGCTTC | 9060 |
| | CAGGGGGAAA CGCCTGGTAT CTTTATAGTC CTGTCGGGTT TCGCCACCTC TGACTTGAGC | 9120 |
| | GTCGATTTTT GTGATGCTCG TCAGGGGGGC GGAGCCTATG GAAAAACGCC AGCAACGCGG | 9180 |
| | CCTTTTTACG GTTCCTGGCC TTTTGCTGGC CTTTTGCTCA CATGTTCTTT CCTGCGTTAT | 9240 |
| | CCCCTGATTC TGTGGATAAC CGTATTACCG CCTTTGAGTG AGCTGATACC GCTCGCCGCA | 9300 |
| | GCCGAACGAC CGAGCGCAGC GAGTCAGTGA GCGAGGAAGC GGAAGAGCGC CCAATACGCA | 9360 |
| | AACCGCCTCT CCCCGCGCGT TGGCCGATTC ATTAATGCAG CTGGCACGAC AGGTTTCCCG | 9420 |
| | ACTGGAAAGC GGGCAGTGAG CGCAACGCAA TTAATGTGAG TTAGCTCACT CATTAGGCAC | 9480 |
| | CCCAGGCTTT ACACTTTATG CTTCCGGCTC GTATGTTGTG TGGAATTGTG AGCGGATAAC | 9540 |
| | AATTTCACAC AGGAAACAGC TATGACCATG ATTACGCCAA GCGCGCAATT AACCCTCACT | 9600 |
| SEQ ID NO: 619 | AATGTAGTCT TATGCAATAC TCTTGTAGTC TTGCAACATG GTAACGATGA GTTAGCAACA | 60 |
| Anti-FAP-V_{L}- | TGCCTTACAA GGAGAGAAAA AGCACCGTGC ATGCCGATTG GTGGAAGTAA GGTGGTACGA | 120 |
| linker- | TCGTGCCTTA TTAGGAAGGC AACAGACGGG TCTGACATGG ATTGGACGAA CCACTGAATT | 180 |
| Anti-FAP-V_{H}- | GCCGCATTGC AGAGATATTG TATTTAAGTG CCTAGCTCGA TACATAAACG GGTCTCTCTG | 240 |
| CD8alpha (hinge | GTTAGACCAG ATCTGAGCCT GGGAGCTCTC TGGCTAACTA GGGAACCCAC TGCTTAAGCC | 300 |
| and | TCAATAAAGC TTGCCTTGAG TGCTTCAAGT AGTGTGTGCC CGTCTGTTGT GTGACTCTGG | 360 |
| transmembrane)- | TAACTAGAGA TCCCTCAGAC CCTTTTAGTC AGTGTGGAAA ATCTCTAGCA GTGGCGCCCG | 420 |
| IL-18R1 | AACAGGGACT TGAAAGCGAA AGGGAAACCA GAGGAGCTCT CTCGACGCAG GACTCGGCTT | 480 |
| | GCTGAAGCGC GCACGGCAAG AGGCGAGGGG CGGCGACTGG TGAGTACGCC AAAAATTTTG | 540 |
| | ACTAGCGGAG GCTAGAAGGA GAGAGATGGG TGCGAGAGCG TCAGTATTAA GCGGGGGAGA | 600 |
| | ATTAGATCGC GATGGGAAAA AATTCGGTTA AGGCCAGGGG GAAAGAAAAA ATATAAATTA | 660 |
| | AAACATATAG TATGGGCAAG CAGGGAGCTA GAACGATTCG CAGTTAATCC TGGCCTGTTA | 720 |
| | GAAACATCAG AAGGCTGTAG ACAAATACTG GGACAGCTAC AACCATCCCT TCAGACAGGA | 780 |
| | TCAGAAGAAC TTAGATCATT ATATAATACA GTAGCAACCC TCTATTGTGT GCATCAAAGG | 840 |
| | ATAGAGATAA AAGACACCAA GGAAGCTTTA GACAAGATAG AGGAAGAGCA AAACAAAAGT | 900 |
| | AAGACCACCG CACAGCAAGC GGCCGCTGAT CTTCAGACCT GGAGGAGGAG ATATGAGGGA | 960 |
| | CAATTGGAGA AGTGAATTAT ATAAATATAA AGTAGTAAAA ATTGAACCAT TAGGAGTAGC | 1020 |
| | ACCCACCAAG GCAAAGAGAA GAGTGGTGCA GAGAGAAAAA AGAGCAGTGG GAATAGGAGC | 1080 |
| | TTTGTTCCTT GGGTTCTTGG GAGCAGCAGG AAGCACTATG GGCGCAGCGT CAATGACGCT | 1140 |
| | GACGGTACAG GCCAGACAAT TATTGTCTGG TATAGTGCAG CAGCAGAACA ATTTGCTGAG | 1200 |
| | GGCTATTGAG GCGCAACAGC ATCTGTTGCA ACTCACAGTC TGGGGCATCA AGCAGCTCCA | 1260 |
| | GGCAAGAATC CTGGCTGTGG AAAGATACCT AAAGGATCAA CAGCTCCTGG GGATTTGGGG | 1320 |
| | TTGCTCTGGA AAACTCATTT GCACCACTGC TGTGCCTTGG AATGCTAGTT GGAGTAATAA | 1380 |
| | ATCTCTGGAA CAGASTTGGA ATCACACGAC CTGGATGGAG TGGGACAGAG AAATTAACAA | 1440 |
| | TTACACAAGC TTAATACACT CCTTAATTGA AGAATCGCAA AACCAGCAAG AAAAGAATGA | 1500 |
| | ACAAGAATTA TTGGAATTAG ATAAATGGGC AAGTTTGTGG AATTGGTTTA ACATAACAAA | 1560 |
| | TTGGCTGTGG TATATAAAAT TATTCATAAT GATAGTAGGA GGCTTGGTAG GTTTAAGAAT | 1620 |
| | AGTTTTTGCT GTACTTTCTA TAGTGAATAG AGTTAGGCAG GGATATTCAC CATTATCGTT | 1680 |
| | TCAGACCCAC CTCCCAACCC CGAGGGGACC CGACAGGCCC GAAGGAATAG AAGAAGAAGG | 1740 |
| | TGGAGAGAGA GACAGAGACA GATCCATTCG ATTAGTGAAC GGATCTCGAC GGTATCGCTA | 1800 |
| | GCTTTTAAAA GAAAAGGGGG GATTGGGGGG TACAGTGCAG GGGAAAGAAT AGTAGACATA | 1860 |
| | ATAGCAACAG ACATACAAAC TAAAGAATTA CAAAAACAAA TTACAAAAAT TCAAAATTTT | 1920 |
| | ACTAGTGATT ATCGGATCAA CTTTGTATAG AAAAGTTGGG CTCCGGTGCC CGTCAGTGGG | 1980 |
| | CAGAGCGCAC ATCGCCCACA GTCCCCGAGA AGTTGGGGGG AGGGGTCGGC AATTGAACCG | 2040 |
| | GTGCCTAGAG AAGGTGGCGC GGGGTAAACT GGGAAAGTGA TGTCGTGTAC TGGCTCCGCC | 2100 |
| | TTTTTCCCGA GGGTGGGGGA GAACCGTATA TAAGTGCAGT AGTCGCCGTG AACGTTCTTT | 2160 |
| | TTCGCAACGG GTTTGCCGCC AGAACACAGG TAAGTGCCGT GTGTGGTTCC CGCGGGCCTG | 2220 |
| | GCCTCTTTAC GGGTTATGGC CCTTGCGTGC CTTGAATTAC TTCCACCTGG CTGCAGTACG | 2280 |
| | TGATTCTTGA TCCCGAGCTT CGGGTTGGAA GTGGGTGGGA GAGTTCGAGG CCTTGCGCTT | 2340 |
| | AAGGAGCCCC TTCGCCTCGT GCTTGAGTTG AGGCCTGGCC TGGGCGCTGG GGCCGCCGCG | 2400 |
| | TGCGAATCTG GTGGCACCTT CGCGCCTGTC TCGCTGCTTT CGATAAGTCT CTAGCCATTT | 2460 |
| | AAAATTTTTG ATGACCTGCT GCGACGCTTT TTTTCTGGCA AGATAGTCTT GTAAATGCGG | 2520 |
| | GCCAAGATCT GCACACTGGT ATTTCGGTTT TTGGGGCCGC GGGCGGCGAC GGGGCCCGTG | 2580 |
| | CGTCCCAGCG CACATGTTCG GCGAGGCGGG GCCTGCGAGC GCGGCCACCG AGAATCGGAC | 2640 |
| | GGGGGTAGTC TCAAGCTGGC CGGCCTGCTC TGGTGCCTGG TCTCGCGCCG CCGTGTATCG | 2700 |
| | CCCCGCCCTG GGCGGCAAGG CTGGCCCGGT CGGCACCAGT TGCGTGAGCG GAAAGATGGC | 2760 |
| | CGCTTCCCGG CCCTGCTGCA GGGAGCTCAA AATGGAGGAC GCGGCGCTCG GGAGAGCGGG | 2820 |
| | CGGGTGAGTC ACCCACACAA AGGAAAAGGG CCTTTCCGTC CTCAGCCGTC GCTTCATGTG | 2880 |
| | ACTCCACGGA GTACCGGGCG CCGTCCAGGC ACCTCGATTA GTTCTCGAGC TTTTGGAGTA | 2940 |
| | CGTCGTCTTT AGGTTGGGGG GAGGGGTTTT ATGCGATGGA GTTTCCCCAC ACTGAGTGGG | 3000 |
| | TGGAGACTGA AGTTAGGCCA GCTTGGCACT TGATGTAATT CTCCTTGGAA TTTGCCCTTT | 3060 |
| | TTGAGTTTGG ATCTTGGTTC ATTCTCAAGC CTCAGACAGT GGTTCAAAGT TTTTTTCTTC | 3120 |
| | CATTTCAGGT GTCGTGACAA GTTTGTACAA AAAAGCAGGC TGCCACCATG GATATTGTGA | 3180 |
| | TGACCCAGAG CCCGGATAGC CTGGCGGTGA GCCTGGGCGA ACGCGCGACC ATTAACTGCA | 3240 |
| | AAAGCAGCCA GAGCCTGCTG TATAGCCGCA ACCAGAAAAA CTATCTGGCG TGGTATCAGC | 3300 |
| | AGAAACCGGG CCAGCCGCCG AAACTGCTGA TTTTTTGGGC GAGCACCCGC GAAAGCGGCG | 3360 |
| | TGCCGGATCG CTTTAGCGGC AGCGGCTTTG GCACCGATTT TACCCTGACC ATTAGCAGCC | 3420 |
| | TGCAGGCGGA AGATGTGGCG GTGTATTATT GCCAGCAGTA TTTTAGCTAT CCGCTGACCT | 3480 |
| | TTGGCCAGGG CACCAAAGTG GAAATTAAAG GCGGCGGCGG CAGCGGCGGC GGCGGCAGCG | 3540 |
| | GCGGCGGCGG CAGCCAGGTG CAGCTGGTGC AGAGCGGCGC GGAAGTGAAA AAACCGGGCG | 3600 |
| | CGAGCGTGAA AGTGAGCTGC AAAACCAGCC GCTATACCTT TACCGAATAT ACCATTCATT | 3660 |
| | GGGTGCGCCA GGCGCCGGGC CAGCGCCTGG AATGGATTGG CGGCATTAAC CCGAACAACG | 3720 |
| | GCATTCCGAA CTATAACCAG AAATTTAAAG GCCGCGTGAC CATTACCGTG GATACCAGCG | 3780 |
| | CGAGCACCGC GTATATGGAA CTGAGCAGCC TGCGCAGCGA AGATACCGCG GTGTATTATT | 3840 |
| | GCGCGCGCCG CCGCATTGCG TATGGCTATG ATGAAGGCCA TGCGATGGAT TATTGGGGCC | 3900 |
| | AGGGCACCCT GGTGACCGTG AGCAGCACCA CGACGCCAGC GCCGCGACCA CCAACACCGG | 3960 |
| | CGCCCACCAT CGCGTCGCAG CCCCTGTCCC TGCGCCCAGA GGCGTGCCGG CCAGCGGCGG | 4020 |
| | GGGGCGCAGT GCACACGAGG GGGCTGGACT TCGCCTGTGA TATCTACATC TGGGCGCCCT | 4080 |
| | TGGCCGGGAC TTGTGGGGTC CTTCTCCTGT CACTGGTTAT CACCCTTTAC TGCTATAGAG | 4140 |
| | TTGACTTGGT TCTATTTTAT AGACATTTAA CGAGAAGAGA TGAAACATTA ACAGATGGAA | 4200 |
| | AAACATATGA TGCTTTTGTG TCTTACCTAA AAGAATGCCG ACCTGAAAAT GGAGAGGAGC | 4260 |
| | ACACCTTTGC TGTGGAGATT TTGCCCAGGG TGTTGGAGAA ACATTTTGGG TATAAGTTAT | 4320 |
| | GCATATTTGA AAGGGATGTA GTGCCTGGAG GAGCTGTTGT TGATGAAATC CACTCACTGA | 4380 |
| | TAGAGAAAAG CCGAAGACTA ATCATTGTCC TAAGTAAAAG TTATATGTCT AATGAGGTCA | 4440 |
| | GGTATGAACT TGAAAGTGGA CTCCATGAAG CATTGGTGGA AAGAAAAATT AAAATAATCT | 4500 |
| | TAATTGAATT TACACCTGTT ACTGACTTCA CATTCTTGCC CCAATCACTA AAGCTTTTGA | 4560 |
| | AATCTCACAG AGTTCTGAAG TGGAAGGCCG ATAAATCTCT TTCTTATAAC TCAAGGTTCT | 4620 |
| | GGAAGAACCT TCTTTACTTA ATGCCTGCAA AAACAGTCAA GCCAGGTAGA GACGAACCGG | 4680 |
| | AAGTCTTGCC TGTTCTTTCC GAGTCTTGAA CCCAGCTTTC TTGTACAAAG TGGTGATAAT | 4740 |
| | CGAATTCCGA TAATCAACCT CTGGATTACA AAATTTGTGA AAGATTGACT GGTATTCTTA | 4800 |
| | ACTATGTTGC TCCTETTACG CTATGTGGAT ACGCTGCTTT AATGCCTTTG TATCATGCTA | 4860 |
| | TTGCTTCCCG TATGGCTTTC ATTTTCTCCT CCTTGTATAA ATCCTGGTTG CTGTCTCTTT | 4920 |
| | ATGAGGAGTT GTGGCCCGTT GTCAGGCAAC GTGGCGTGGT GTGCACTGTG TTTGCTGACG | 4980 |
| | CAACCCCCAC TGGTTGGGGC ATTGCCACCA CCTGTCAGCT CCTTTCCGGG ACTTTCGCTT | 5040 |
| | TCCCCCTCCC TATTGCCACG GCGGAACTCA TCGCCGCCTG CCTTGCCCGC TGCTGGACAG | 5100 |
| | GGGCTCGGCT GTTGGGCACT GACAATTCCG TGGTGTTGTC GGGGAAGCTG ACGTCCTTTC | 5160 |
| | CATGGCTGCT CGCCTGTGTT GCCACCTGGA TTCTGCGCGG GACGTCCTTC TGCTACGTCC | 5220 |
| | CTTCGGCCCT CAATCCAGCG GACCTTCCTT CCCGCGGCCT GCTGCCGGCT CTGCGGCCTC | 5280 |
| | TTCCGCGTCT TCGCCTTCGC CCTCAGACGA GTCGGATCTC CCTTTGGGCC GCCTCCCCGC | 5340 |
| | ATCGGGAATT CCCGCGGTTC GCTTTAAGAC CAATGACTTA CAAGGCAGCT GTAGATCTTA | 5400 |
| | GCCACTTTTT AAAAGAAAAG GGGGGACTGG AAGGGCTAAT TCACTCCCAA CGAAGACAAG | 5460 |
| | ATCTGCTTTT TGCTTGTACT GGGTCTCTCT GGTTAGACCA GATCTGAGCC TGGGAGCTCT | 5520 |
| | CTGGCTAACT AGGGAACCCA CTGCTTAAGC CTCAATAAAG CTTGCCTTGA GTGCTTCAAG | 5580 |
| | TAGTGTGTGC CCGTCTGTTG TGTGACTCTG GTAACTAGAG ATCCCTCAGA CCCTTTTAGT | 5640 |
| | CAGTGTGGAA AATCTCTAGC AGTAGTAGTT CATGTCATCT TATTATTCAG TATTTATAAC | 5700 |
| | TTGCAAAGAA ATGAATATCA GAGAGTGAGA GGAACTTGTT TATTGCAGCT TATAATGGTT | 5760 |
| | ACAAATAAAG CAATAGCATC ACAAATTTCA CAAATAAAGC ATTTTTTTCA CTGCATTCTA | 5820 |
| | GTTGTGGTTT GTCCAAACTC ATCAATGTAT CTTATCATGT CTGGCTCTAG CTATCCCGCC | 5880 |
| | CCTAACTCCG CCCATCCCGC CCCTAACTCC GCCCAGTTCC GCCCATTCTC CGCCCCATGG | 5940 |
| | CTGACTAATT TTTTTTATTT ATGCAGAGGC CGAGGCCGCC TCGGCCTCTG AGCTATTCCA | 6000 |
| | GAAGTAGTGA GGAGGCTTTT TTGGAGGCCT AGGGACGTAC CCAATTCGCC CTATAGTGAG | 6060 |
| | TCGTATTACG CGCGCTCACT GGCCGTCGTT TTACAACGTC GTGACTGGGA AAACCCTGGC | 6120 |
| | GTTACCCAAC TTAATCGCCT TGCAGCACAT CCCCCTTTCG CCAGCTGGCG TAATAGCGAA | 6180 |
| | GAGGCCCGCA CCGATCGCCC TTCCCAACAG TTGCGCAGCC TGAATGGCGA ATGGGACGCG | 6240 |
| | CCCTGTAGCG GCGCATTAAG CGCGGCGGGT GTGGTGGTTA CGCGCAGCGT GACCGCTACA | 6300 |
| | CTTGCCAGCG CCCTAGCGCC CGCTCCTTTC GCTTTCTTCC CTTCCTTTCT CGCCACGTTC | 6360 |
| | GCCGGCTTTC CCCGTCAAGC TCTAAATCGG GGGCTCCCTT TAGGGTTCCG ATTTAGTGCT | 6420 |
| | TTACGGCACC TCGACCCCAA AAAACTTGAT TAGGGTGATG GTTCACGTAG TGGGCCATCG | 6480 |
| | CCCTGATAGA CGGTTTTTCG CCCTTTGACG TTGGAGTCCA CGTTCTTTAA TAGTGGACTC | 6540 |
| | TTGTTCCAAA CTGGAACAAC ACTCAACCCT ATCTCGGTCT ATTCTTTTGA TTTATAAGGG | 6600 |
| | ATTTTGCCGA TTTCGGCCTA TTGGTTAAAA AATGAGCTGA TTTAACAAAA ATTTAACGCG | 6660 |
| | AATTTTAACA AAATATTAAC GCTTACAATT TAGGTGGCAC TTTTCGGGGA AATGTGCGCG | 6720 |
| | GAACCCCTAT TTGTTTATTT TTCTAAATAC ATTCAAATAT GTATCCGCTC ATGAGACAAT | 6780 |
| | AACCCTGATA AATGCTTCAA TAATATTGAA AAAGGAAGAG TATGAGTATT CAACATTTCC | 6840 |
| | GTGTCGCCCT TATTCCCTTT TTTGCGGCAT TTTGCCTTCC TGTTTTTGCT CACCCAGAAA | 6900 |
| | CGCTGGTGAA AGTAAAAGAT GCTGAAGATC AGTTGGGTGC ACGAGTGGGT TACATCGAAC | 6960 |
| | TGGATCTCAA CAGCGGTAAG ATCCTTGAGA GTTTTCGCCC CGAAGAACGT TTTCCAATGA | 7020 |
| | TGAGCACTTT TAAAGTTCTG CTATGTGGCG CGGTATTATC CCGTATTGAC GCCGGGCAAG | 7080 |
| | AGCAACTCGG TCGCCGCATA CACTATTCTC AGAATGACTT GGTTGAGTAC TCACCAGTCA | 7140 |
| | CAGAAAAGCA TCTTACGGAT GGCATGACAG TAAGAGAATT ATGCAGTGCT GCCATAACCA | 7200 |
| | TGAGTGATAA CACTGCGGCC AACTTACTTC TGACAACGAT CGGAGGACCG AAGGAGCTAA | 7260 |
| | CCGCTTTTTT GCACAACATG GGGGATCATG TAACTCGCCT TGATCGTTGG GAACCGGAGC | 7320 |
| | TGAATGAAGC CATACCAAAC GACGAGCGTG ACACCACGAT GCCTGTAGCA ATGGCAACAA | 7380 |
| | CGTTGCGCAA ACTASTAACT GGCGAACTAC TTACTCTAGC TTCCCGGCAA CAATTAATAG | 7440 |
| | ACTGGATGGA GGCGGATAAA GTTGCAGGAC CACTTCTGCG CTCGGCCCTT CCGGCTGGCT | 7500 |
| | GGTTTATTGC TGATAAATCT GGAGCCGGTG AGCGTGGGTC TCGCGGTATC ATTGCAGCAC | 7560 |
| | TGGGGCCAGA TGGTAAGCCC TCCCGTATCG TAGTTATCTA CACGACGGGG AGTCAGGCAA | 7620 |
| | CTATGGATGA ACGAAATAGA CAGATCGCTG AGATAGGTGC CTCACTGATT AAGCATTGGT | 7680 |
| | AACTGTCAGA CCAAGTTTAC TCATATATAC TTTAGATTGA TTTAAAACTT CATTTTTAAT | 7740 |
| | TTAAAAGGAT CTAGGTGAAG ATCCTTTTTG ATAATCTCAT GACCAAAATC CCTTAACGTG | 7800 |
| | AGTTTTCGTT CCACTGAGCG TCAGACCCCG TAGAAAAGAT CAAAGGATCT TCTTGAGATC | 7860 |
| | CTTTTTTTCT GCGCGTAATC TGCTGCTTGC AAACAAAAAA ACCACCGCTA CCAGCGGTGG | 7920 |
| | TTTGTTTGCC GGATCAAGAG CTACCAACTC TTTTTCCGAA GGTAACTGGC TTCAGCAGAG | 7980 |
| | CGCAGATACC AAATACTGTT CTTCTAGTGT AGCCGTAGTT AGGCCACCAC TTCAAGAACT | 8040 |
| | CTGTAGCACC GCCTACATAC CTCGCTCTGC TAATCCTGTT ACCAGTGGCT GCTGCCAGTG | 8100 |
| | GCGATAAGTC GTGTCTTACC GGGTTGGACT CAAGACGATA GTTACCGGAT AAGGCGCAGC | 8160 |
| | GGTCGGGCTG AACGGGGGGT TCGTGCACAC AGCCCAGCTT GGAGCGAACG ACCTACACCG | 8220 |
| | AACTGAGATA CCTACAGCGT GAGCTATGAG AAAGCGCCAC GCTTCCCGAA GAGAGAAAGG | 8280 |
| | CGGACAGGTA TCCGGTAAGC GGCAGGGTCG GAACAGGAGA GCGCACGAGG GAGCTTCCAG | 8340 |
| | GGGGAAACGC CTGGTATCTT TATAGTCCTG TCGGGTTTCG CCACCTCTGA CTTGAGCGTC | 8400 |
| | GATTTTTGTG ATGCTCGTCA GGGGGGCGGA GCCTATGGAA AAACGCCAGC AACGCGGCCT | 8460 |
| | TTTTACGGTT CCTGGCCTTT TGCTGGCCTT TTGCTCACAT GTTCTTTCCT GCGTTATCCC | 8520 |
| | CTGATTCTGT GGATAACCGT ATTACCGCCT TTGAGTGAGC TGATACCGCT CGCCGCAGCC | 8580 |
| | GAACGACCGA GCGCAGCGAG TCAGTGAGCG AGGAAGCGGA AGAGCGCCCA ATACGCAAAC | 8640 |
| | CGCCTCTCCC CGCGCGTTGG CCGATTCATT AATGCAGCTG GCACGACAGG TTTCCCGACT | 8700 |
| | GGAAAGCGGG CAGTGAGCGC AACGCAATTA ATGTGAGTTA GCTCACTCAT TAGGCACCCC | 8760 |
| | AGGCTTTACA CTTTATGCTT CCGGCTCGTA TGTTGTGTGG AATTGTGAGC GGATAACAAT | 8820 |
| | TTCACACAGG AAACAGCTAT GACCATGATT ACGCCAAGCG CGCAATTAAC CCTCACTAAA | 8880 |
| | GGGAACAAAA GCTGGAGCTG CAAGCTT | 8907 |
| SEQ ID NO: 620 | AATGTAGTCT TATGCAATAC TCTTGTAGTC TTGCAACATG GTAACGATGA GTTAGCAACA | 60 |
| Anti-PD-L1-V_{L}- | TGCCTTACAA GGAGAGAAAA AGCACCGTGC ATGCCGATTG GTGGAAGTAA GGTGGTACGA | 120 |
| linker-Anti-PD- | TCGTGCCTTA TTAGGAAGGC AACAGACGGG TCTGACATGG ATTGGACGAA CCACTGAATT | 180 |
| L1-V_{H} (38A1)- | GCCGCATTGC AGAGATATTG TATTTAAGTG CCTAGCTCGA TACATAAACG GGTCTCTCTG | 240 |
| CD8alpha (hinge | GTTAGACCAG ATCTGAGCCT GGGAGCTCTC TGGCTAACTA GGGAACCCAC TGCTTAAGCC | 300 |
| and | TCAATAAAGC TTGCCTTGAG TGCTTCAAGT AGTGTGTGCC CGTCTGTTGT GTGACTCTGG | 360 |
| transmembrane)- | TAACTAGAGA TCCCTCAGAC CCTTTTAGTC AGTGTGGAAA ATCTCTAGCA GTGGCGCCCG | 420 |
| CD27 | AACAGGGACT TGAAAGCGAA AGGGAAACCA GAGGAGCTCT CTCGACGCAG GACTCGGCTT | 480 |
| | GCTGAAGCGC GCACGGCAAG AGGCGAGGGG CGGCGACTGG TGAGTACGCC AAAAATTTTG | 540 |
| | ACTAGCGGAG GCTAGAAGGA GAGAGATGGG TGCGAGAGCG TCAGTATTAA GCGGGGGAGA | 600 |
| | ATTAGATCGC GATGGGAAAA AATTCGGTTA AGGCCAGGGG GAAAGAAAAA ATATAAATTA | 660 |
| | AAACATATAG TATGGGCAAG CAGGGAGCTA GAACGATTCG CAGTTAATCC TGGCCTGTTA | 720 |
| | GAAACATCAG AAGGCTGTAG ACAAATACTG GGACAGCTAC AACCATCCCT TCAGACAGGA | 780 |
| | TCAGAAGAAC TTAGATCATT ATATAATACA GTAGCAACCC TCTATTGTGT GCATCAAAGG | 840 |
| | ATAGAGATAA AAGACACCAA GGAAGCTTTA GACAAGATAG AGGAAGAGCA AAACAAAAGT | 900 |
| | AAGACCACCG CACAGCAAGC GGCCGCTGAT CTTCAGACCT GGAGGAGGAG ATATGAGGGA | 960 |
| | CAATTGGAGA AGTGAATTAT ATAAATATAA AGTAGTAAAA ATTGAACCAT TAGGAGTAGC | 1020 |
| | ACCCACCAAG GCAAAGAGAA GAGTGGTGCA GAGAGAAAAA AGAGCAGTGG GAATAGGAGC | 1080 |
| | TTTGTTCCTT GGGTTCTTGG GAGCAGCAGG AAGCACTATG GGCGCAGCGT CAATGACGCT | 1140 |
| | GACGGTACAG GCCAGACAAT TATTGTCTGG TATAGTGCAG CAGCAGAACA ATTTGCTGAG | 1200 |
| | GGCTATTGAG GCGCAACAGC ATCTGTTGCA ACTCACAGTC TGGGGCATCA AGCAGCTCCA | 1260 |
| | GGCAAGAATC CTGGCTGTGG AAAGATACCT AAAGGATCAA CAGCTCCTGG GGATTTGGGG | 1320 |
| | TTGCTCTGGA AAACTCATTT GCACCACTGC TGTGCCTTGG AATGCTAGTT GGAGTAATAA | 1380 |
| | ATCTCTGGAA CAGASTTGGA ATCACACGAC CTGGATGGAG TGGGACAGAG AAATTAACAA | 1440 |
| | TTACACAAGC TTAATACACT CCTTAATTGA AGAATCGCAA AACCAGCAAG AAAAGAATGA | 1500 |
| | ACAAGAATTA TTGGAATTAG ATAAATGGGC AAGTTTGTGG AATTGGTTTA ACATAACAAA | 1560 |
| | TTGGCTGTGG TATATAAAAT TATTCATAAT GATAGTAGGA GGCTTGGTAG GTTTAAGAAT | 1620 |
| | AGTTTTTGCT GTACTTTCTA TAGTGAATAG AGTTAGGCAG GGATATTCAC CATTATCGTT | 1680 |
| | TCAGACCCAC CTCCCAACCC CGAGGGGACC CGACAGGCCC GAAGGAATAG AAGAAGAAGG | 1740 |
| | TGGAGAGAGA GACAGAGACA GATCCATTCG ATTAGTGAAC GGATCTCGAC GGTATCGCTA | 1800 |
| | GCTTTTAAAA GAAAAGGGGG GATTGGGGGG TACAGTGCAG GGGAAAGAAT AGTAGACATA | 1860 |
| | ATAGCAACAG ACATACAAAC TAAAGAATTA CAAAAACAAA TTACAAAAAT TCAAAATTTT | 1920 |
| | ACTAGTGATT ATCGGATCAA CTTTGTATAG AAAAGTTGGG CTCCGGTGCC CGTCAGTGGG | 1980 |
| | CAGAGCGCAC ATCGCCCACA GTCCCCGAGA AGTTGGGGGG AGGGGTCGGC AATTGAACCG | 2040 |
| | GTGCCTAGAG AAGGTGGCGC GGGGTAAACT GGGAAAGTGA TGTCGTGTAC TGGCTCCGCC | 2100 |
| | TTTTTCCCGA GGGTGGGGGA GAACCGTATA TAAGTGCAGT AGTCGCCGTG AACGTTCTTT | 2160 |
| | TTCGCAACGG GTTTGCCGCC AGAACACAGG TAAGTGCCGT GTGTGGTTCC CGCGGGCCTG | 2220 |
| | GCCTCTTTAC GGGTTATGGC CCTTGCGTGC CTTGAATTAC TTCCACCTGG CTGCAGTACG | 2280 |
| | TGATTCTTGA TCCCGAGCTT CGGGTTGGAA GTGGGTGGGA GAGTTCGAGG CCTTGCGCTT | 2340 |
| | AAGGAGCCCC TTCGCCTCGT GCTTGAGTTG AGGCCTGGCC TGGGCGCTGG GGCCGCCGCG | 2400 |
| | TGCGAATCTG GTGGCACCTT CGCGCCTGTC TCGCTGCTTT CGATAAGTCT CTAGCCATTT | 2460 |
| | AAAATTTTTG ATGACCTGCT GCGACGCTTT TTTTCTGGCA AGATAGTCTT GTAAATGCGG | 2520 |
| | GCCAAGATCT GCACACTGGT ATTTCGGTTT TTGGGGCCGC GGGCGGCGAC GGGGCCCGTG | 2580 |
| | CGTCCCAGCG CACATGTTCG GCGAGGCGGG GCCTGCGAGC GCGGCCACCG AGAATCGGAC | 2640 |
| | GGGGGTAGTC TCAAGCTGGC CGGCCTGCTC TGGTGCCTGG TCTCGCGCCG CCGTGTATCG | 2700 |
| | CCCCGCCCTG GGCGGCAAGG CTGGCCCGGT CGGCACCAGT TGCGTGAGCG GAAAGATGGC | 2760 |
| | CGCTTCCCGG CCCTGCTGCA GGGAGCTCAA AATGGAGGAC GCGGCGCTCG GGAGAGCGGG | 2820 |
| | CGGGTGAGTC ACCCACACAA AGGAAAAGGG CCTTTCCGTC CTCAGCCGTC GCTTCATGTG | 2880 |
| | ACTCCACGGA GTACCGGGCG CCGTCCAGGC ACCTCGATTA GTTCTCGAGC TTTTGGAGTA | 2940 |
| | CGTCGTCTTT AGGTTGGGGG GAGGGGTTTT ATGCGATGGA GTTTCCCCAC ACTGAGTGGG | 3000 |
| | TGGAGACTGA AGTTAGGCCA GCTTGGCACT TGATGTAATT CTCCTTGGAA TTTGCCCTTT | 3060 |
| | TTGAGTTTGG ATCTTGGTTC ATTCTCAAGC CTCAGACAGT GGTTCAAAGT TTTTTTCTTC | 3120 |
| | CATTTCAGGT GTCGTGACAA GTTTGTACAA AAAAGCAGGC TGCCACCATG AGCTATGTGC | 3180 |
| | TGACCCAGCC GCCGAGCGTG AGCGTGGCGC CGGGCCAGAC CGCGCGCATT ACCTGCGGCG | 3240 |
| | GCAACAACAT TGGCCGCAAA ATTGTGCATT GGTATCAGCA GCGCCCGGGC CAGGCGCCGG | 3300 |
| | TGCTGGTGAT TTATTATGAT ACCGATCGCC CGGCGGGCAT TCCGGAACGC TTTAGCGGCA | 3360 |
| | GCAACAGCGG CAACATGGCG ACCCTGACCA TTAGCACCGT GGGCGCGGGC GATGAAGCGG | 3420 |
| | ATTATTATTG CCAGGTGTGG GATACCGGCA GCGATCATGT GGTGTTTGGC GGCGGCACCA | 3480 |
| | AACTGACCGT GCTGGGCGGC GGCGGCAGCG GCGGCGGCGG CAGCGGCGGC GGCGGCAGCG | 3540 |
| | AAGTGCAGCT GGTGGAAAGC GGCGGCGGCC TGGTGCAGCC GGGCGGCAGC CTGCGCCTGA | 3600 |
| | GCTGCGCGGC GAGCGGCTTT ACCTTTAGCA ACTATGCGAT GAGCTGGGTG CGCCAGGCGC | 3660 |
| | CGGGCAAAGG CCTGGAATGG GTGAGCACCA TTAGCGGCAG CGGCGGCACC ACCTATTATG | 3720 |
| | CGGATAGCGT GAAAGGCCGC TTTACCATTA GCCGCGATAA CAGCAAAAAC ACCCTGTATC | 3780 |
| | TGCAGATGAA CAGCCTGCGC GTGGAAGATA CCGCGGTGTA TTATTGCGCG AAAGATTGGT | 3840 |
| | TTCGCAGCAG CAGCCCGGAT GCGTTTGATA TTTGGGGCCA GGGCACCACC GTGACCGTGA | 3900 |
| | GCGCGACCAC GACGCCAGCG CCGCGACCAC CAACACCGGC GCCCACCATC GCGTCGCAGC | 3960 |
| | CCCTGTCCCT GCGCCCAGAG GCGTGCCGGC CAGCGGCGGG GGGCGCAGTG CACACGAGGG | 4020 |
| | GGCTGGACTT CGCCTGTGAT ATCTACATCT GGGCGCCCTT GGCCGGGACT TGTGGGGTCC | 4080 |
| | TTCTCCTGTC ACTGGTTATC ACCCTTTACT GCCAACGAAG GAAATATAGA TCAAACAAAG | 4140 |
| | GAGAAAGTCC TGTGGAGCCT GCAGAGCCTT GTCGTTACAG CTGCCCCAGG GAGGAGGAGG | 4200 |
| | GCAGCACCAT CCCCATCCAG GAGGATTACC GAAAACCGGA GCCTGCCTGC TCCCCCTGAA | 4260 |
| | CCCAGCTTTC TTGTACAAAG TGGTGATAAT CGAATTCCGA TAATCAACCT CTGGATTACA | 4320 |
| | AAATTTGTGA AAGATTGACT GGTATTCTTA ACTATGTTGC TCCTTTTACG CTATGTGGAT | 4380 |
| | ACGCTGCTTT AATGCCTTTG TATCATGCTA TTGCTTCCCG TATGGCTTTC ATTTTCTCCT | 4440 |
| | CCTTGTATAA ATCCTGGTTG CTGTCTCTTT ATGAGGAGTT GTGGCCCGTT GTCAGGCAAC | 4500 |
| | GTGGCGTGGT GTGCACTGTG TTTGCTGACG CAACCCCCAC TGGTTGGGGC ATTGCCACCA | 4560 |
| | CCTGTCAGCT CCTTTCCGGG ACTTTCGCTT TCCCCCTCCC TATTGCCACG GCGGAACTCA | 4620 |
| | TCGCCGCCTG CCTTGCCCGC TGCTGGACAG GGGCTCGGCT GTTGGGCACT GACAATTCCG | 4680 |
| | TGGTGTTGTC GGGGAAGCTG ACGTCCTTTC CATGGCTGCT CGCCTGTGTT GCCACCTGGA | 4740 |
| | TTCTGCGCGG GACGTCCTTC TGCTACGTCC CTTCGGCCCT CAATCCAGCG GACCTTCCTT | 4800 |
| | CCCGCGGCCT GCTGCCGGCT CTGCGGCCTC TTCCGCGTCT TCGCCTTCGC CCTCAGACGA | 4860 |
| | GTCGGATCTC CCTTTGGGCC GCCTCCCCGC ATCGGGAATT CCCGCGGTTC GCTTTAAGAC | 4920 |
| | CAATGACTTA CAAGGCAGCT GTAGATCTTA GCCACTTTTT AAAAGAAAAG GGGGGACTGG | 4980 |
| | AAGGGCTAAT TCACTCCCAA CGAAGACAAG ATCTGCTTTT TGCTTGTACT GGGTCTCTCT | 5040 |
| | GGTTAGACCA GATCTGAGCC TGGGAGCTCT CTGGCTAACT AGGGAACCCA CTGCTTAAGC | 5100 |
| | CTCAATAAAG CTTGCCTTGA GTGCTTCAAG TAGTGTGTGC CCGTCTGTTG TGTGACTCTG | 5160 |
| | GTAACTAGAG ATCCCTCAGA CCCTTTTAGT CAGTGTGGAA AATCTCTAGC AGTAGTAGTT | 5220 |
| | CATGTCATCT TATTATTCAG TATTTATAAC TTGCAAAGAA ATGAATATCA GAGAGTGAGA | 5280 |
| | GGAACTTGTT TATTGCAGCT TATAATGGTT ACAAATAAAG CAATAGCATC ACAAATTTCA | 5340 |
| | CAAATAAAGC ATTTTTTTCA CTGCATTCTA GTTGTGGTTT GTCCAAACTC ATCAATGTAT | 5400 |
| | CTTATCATGT CTGGCTCTAG CTATCCCGCC CCTAACTCCG CCCATCCCGC CCCTAACTCC | 5460 |
| | GCCCAGTTCC GCCCATTCTC CGCCCCATGG CTGACTAATT TTTTTTATTT ATGCAGAGGC | 5520 |
| | CGAGGCCGCC TCGGCCTCTG AGCTATTCCA GAAGTAGTGA GGAGGCTTTT TTGGAGGCCT | 5580 |
| | AGGGACGTAC CCAATTCGCC CTATAGTGAG TCGTATTACG CGCGCTCACT GGCCGTCGTT | 5640 |
| | TTACAACGTC GTGACTGGGA AAACCCTGGC GTTACCCAAC TTAATCGCCT TGCAGCACAT | 5700 |
| | CCCCCTTTCG CCAGCTGGCG TAATAGCGAA GAGGCCCGCA CCGATCGCCC TTCCCAACAG | 5760 |
| | TTGCGCAGCC TGAATGGCGA ATGGGACGCG CCCTGTAGCG GCGCATTAAG CGCGGCGGGT | 5820 |
| | GTGGTGGTTA CGCGCAGCGT GACCGCTACA CTTGCCAGCG CCCTAGCGCC CGCTCCTTTC | 5880 |
| | GCTTTCTTCC CTTCCTTTCT CGCCACGTTC GCCGGCTTTC CCCGTCAAGC TCTAAATCGG | 5940 |
| | GGGCTCCCTT TAGGGTTCCG ATTTAGTGCT TTACGGCACC TCGACCCCAA AAAACTTGAT | 6000 |
| | TAGGGTGATG GTTCACGTAG TGGGCCATCG CCCTGATAGA CGGTTTTTCG CCCTTTGACG | 6060 |
| | TTGGAGTCCA CGTTCTTTAA TAGTGGACTC TTGTTCCAAA CTGGAACAAC ACTCAACCCT | 6120 |
| | ATCTCGGTCT ATTCTTTTGA TTTATAAGGG ATTTTGCCGA TTTCGGCCTA TTGGTTAAAA | 6180 |
| | AATGAGCTGA TTTAACAAAA ATTTAACGCG AATTTTAACA AAATATTAAC GCTTACAATT | 6240 |
| | TAGGTGGCAC TTTTCGGGGA AATGTGCGCG GAACCCCTAT TTGTTTATTT TTCTAAATAC | 6300 |
| | ATTCAAATAT GTATCCGCTC ATGAGACAAT AACCCTGATA AATGCTTCAA TAATATTGAA | 6360 |
| | AAAGGAAGAG TATGAGTATT CAACATTTCC GTGTCGCCCT TATTCCCTTT TTTGCGGCAT | 6420 |
| | TTTGCCTTCC TGTTTTTGCT CACCCAGAAA CGCTGGTGAA AGTAAAAGAT GCTGAAGATC | 6480 |
| | AGTTGGGTGC ACGAGTGGGT TACATCGAAC TGGATCTCAA CAGCGGTAAG ATCCTTGAGA | 6540 |
| | GTTTTCGCCC CGAAGAACGT TTTCCAATGA TGAGCACTTT TAAAGTTCTG CTATGTGGCG | 6600 |
| | CGGTATTATC CCGTATTGAC GCCGGGCAAG AGCAACTCGG TCGCCGCATA CACTATTCTC | 6660 |
| | AGAATGACTT GGTTGAGTAC TCACCAGTCA CAGAAAAGCA TCTTACGGAT GGCATGACAG | 6720 |
| | TAAGAGAATT ATGCAGTGCT GCCATAACCA TGAGTGATAA CACTGCGGCC AACTTACTTC | 6780 |
| | TGACAACGAT CGGAGGACCG AAGGAGCTAA CCGCTTTTTT GCACAACATG GGGGATCATG | 6840 |
| | TAACTCGCCT TGATCGTTGG GAACCGGAGC TGAATGAAGC CATACCAAAC GACGAGCGTG | 6900 |
| | ACACCACGAT GCCTGTAGCA ATGGCAACAA CGTTGCGCAA ACTATTAACT GGCGAACTAC | 6960 |
| | TTACTCTAGC TTCCCGGCAA CAATTAATAG ACTGGATGGA GGCGGATAAA GTTGCAGGAC | 7020 |
| | CACTTCTGCG CTCGGCCCTT CCGGCTGGCT GGTTTATTGC TGATAAATCT GGAGCCGGTG | 7080 |
| | AGCGTGGGTC TCGCGGTATC ATTGCAGCAC TGGGGCCAGA TGGTAAGCCC TCCCGTATCG | 7140 |
| | TAGTTATCTA CACGACGGGG AGTCAGGCAA CTATGGATGA ACGAAATAGA CAGATCGCTG | 7200 |
| | AGATAGGTGC CTCACTGATT AAGCATTGGT AACTGTCAGA CCAAGTTTAC TCATATATAC | 7260 |
| | TTTAGATTGA TTTAAAACTT CATTTTTAAT TTAAAAGGAT CTAGGTGAAG ATCCTTTTTG | 7320 |
| | ATAATCTCAT GACCAAAATC CCTTAACGTG AGTTTTCGTT CCACTGAGCG TCAGACCCCG | 7380 |
| | TAGAAAAGAT CAAAGGATCT TCTTGAGATC CTTTTTTTCT GCGCGTAATC TGCTGCTTGC | 7440 |
| | AAACAAAAAA ACCACCGCTA CCAGCGGTGG TTTGTTTGCC GGATCAAGAG CTACCAACTC | 7500 |
| | TTTTTCCGAA GGTAACTGGC TTCAGCAGAG CGCAGATACC AAATACTGTT CTTCTAGTGT | 7560 |
| | AGCCGTAGTT AGGCCACCAC TTCAAGAACT CTGTAGCACC GCCTACATAC CTCGCTCTGC | 7620 |
| | TAATCCTGTT ACCAGTGGCT GCTGCCAGTG GCGATAAGTC GTGTCTTACC GGGTTGGACT | 7680 |
| | CAAGACGATA GTTACCGGAT AAGGCGCAGC GGTCGGGCTG AACGGGGGGT TCGTGCACAC | 7740 |
| | AGCCCAGCTT GGAGCGAACG ACCTACACCG AACTGAGATA CCTACAGCGT GAGCTATGAG | 7800 |
| | AAAGCGCCAC GCTTCCCGAA GAGAGAAAGG CGGACAGGTA TCCGGTAAGC GGCAGGGTCG | 7860 |
| | GAACAGGAGA GCGCACGAGG GAGCTTCCAG GGGGAAACGC CTGGTATCTT TATAGTCCTG | 7920 |
| | TCGGGTTTCG CCACCTCTGA CTTGAGCGTC GATTTTTGTG ATGCTCGTCA GGGGGGCGGA | 7980 |
| | GCCTATGGAA AAACGCCAGC AACGCGGCCT TTTTACGGTT CCTGGCCTTT TGCTGGCCTT | 8040 |
| | TTGCTCACAT GTTCTTTCCT GCGTTATCCC CTGATTCTGT GGATAACCGT ATTACCGCCT | 8100 |
| | TTGAGTGAGC TGATACCGCT CGCCGCAGCC GAACGACCGA GCGCAGCGAG TCAGTGAGCG | 8160 |
| | AGGAAGCGGA AGAGCGCCCA ATACGCAAAC CGCCTCTCCC CGCGCGTTGG CCGATTCATT | 8220 |
| | AATGCAGCTG GCACGACAGG TTTCCCGACT GGAAAGCGGG CAGTGAGCGC AACGCAATTA | 8280 |
| | ATGTGAGTTA GCTCACTCAT TAGGCACCCC AGGCTTTACA CTTTATGCTT CCGGCTCGTA | 8340 |
| | TGTTGTGTGG AATTGTGAGC GGATAACAAT TTCACACAGG AAACAGCTAT GACCATGATT | 8400 |
| | ACGCCAAGCG CGCAATTAAC CCTCACTAAA GGGAACAAAA GCTGGAGCTG CAAGCTT | 8457 |
| SEQ ID NO: 621 | GTCGACGGAT CGGGAGATCT CCCGATCCCC TATGGTGCAC TCTCAGTACA ATCTGCTCTG | 60 |
| pLentiCas9-EGFP | ATGCCGCATA GTTAAGCCAG TATCTGCTCC CTGCTTGTGT GTTGGAGGTC GCTGAGTAGT | 120 |
| vector | GCGCGAGCAA AATTTAAGCT ACAACAAGGC AAGGCTTGAC CGACAATTGC ATGAAGAATC | 180 |
| biepitope CCR | TGCTTAGGGT TAGGCGTTTT GCGCTGCTTC GCGATGTACG GGCCAGATAT ACGCGTTGAC | 240 |
| SP-(38A1 scFv)- | ATTGATTATT GACTAGTTAT TAATAGTAAT CAATTACGGG GTCATTAGTT CATAGCCCAT | 300 |
| (CD28 hinge and | ATATGGAGTT CCGCGTTACA TAACTTACGG TAAATGGCCC GCCTGGCTGA CCGCCCAACG | 360 |
| transmembrane)- | ACCCCCGCCC ATTGACGTCA ATAATGACGT ATGTTCCCAT AGTAACGCCA ATAGGGACTT | 420 |
| (IL-2Rβ | TCCATTGACG TCAATGGGTG GAGTATTTAC GGTAAACTGC CCACTTGGCA GTACATCAAG | 480 |
| intracellular)- | TGTATCATAT GCCAAGTACG CCCCCTATTG ACGTCAATGA CGGTAAATGG CCCGCCTGGC | 540 |
| T2A-SP-(19H9 | ATTATGCCCA GTACATGACC TTATGGGACT TTCCTACTTG GCAGTACATC TACGTATTAG | 600 |
| SCEv)-(CD28 | TCATCGCTAT TACCATGGTG ATGCGGTTTT GGCAGTACAT CAATGGGCGT GGATAGCGGT | 660 |
| hinge and | TTGACTCACG GGGASTTCCA AGTCTCCACC CCATTGACGT CAATGGGAGT TTGTTTTGGC | 720 |
| transmembrane)- | ACCAAAATCA ACGGGACTTT CCAAAATGTC GTAACAACTC CGCCCCATTG ACGCAAATGG | 780 |
| (IL-2Rγ | GCGGTAGGCG TGTACGGTGG GAGGTCTATA TAAGCAGCGC GTTTTGCCTG TACTGGGTCT | 840 |
| intracellular) | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 186C |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TACCTGTGAC CGCTCTGCTG CTGCCTCTGG CCCTGCTGCT | 2940 |
| | GCACGCCGCT AGACCAAGCT ACGTGCTGAC GCAGCCTCCT TCTGTGTCTG TGGCACCTGG | 3000 |
| | TCAGACCGCC CGGATAACAT GCGGCGGCAA CAACATCGGC CGGAAGATCG TGCACTGGTA | 3060 |
| | TCAGCAGCGG CCTGGGCAGG CTCCTGTGCT GGTTATCTAC TACGACACCG ACAGACCTGC | 3120 |
| | CGGCATCCCT GAGCGGTTTA GCGGCAGCAA CAGCGGCAAC ATGGCTACAC TGACAATCAG | 3180 |
| | CACCGTTGGC GCTGGCGACG AAGCCGACTA CTACTGCCAA GTGTGGGACA CAGGCTCTGA | 3240 |
| | CCACGTTGTG TTCGGCGGCG GCACCAAGCT GACCGTGCTG GGCGGCGGTG GCTCTGGCGG | 3300 |
| | CGGCGGATCC GGCGGAGGCG GCAGCGGCGG CGGCGGCTCG GAAGTGCAGC TGGTGGAAAG | 3360 |
| | CGGGGGGGGC CTCGETCAGC CAGGAGGCAG CCTGAGACTG TCTTGCGCCG CTTCTGGCTT | 3420 |
| | TACCTTTTCC AACTACGCCA TGAGCTGGGT GAGGCAGGCC CCCGGCAAAG GCCTTGAGTG | 3480 |
| | GGTGAGCACC ATCTCCGGCT CTGGAGGCAC CACCTACTAC GCCGACTCCG TGAAAGGCAG | 3540 |
| | ATTCACCATC AGCAGAGATA ATAGCAAGAA CACCCTGTAC CTGCAGATGA ACTCCCTGAG | 3600 |
| | AGTGGAAGAT ACCGCCGTGT ACTACTGTGC CAAGGACTGG TTCCGGAGCA GCAGCCCTGA | 3660 |
| | TGCCTTCGAC ATCTGGGGCC AGGGCACAAC CGTGACCGTG TCTGCCATCG AGGTGATGTA | 3720 |
| | CCCTCCACCC TACCTGGACA ACGAGAAGAG CAACGGCACA ATCATCCACG TGAAAGGAAA | 3780 |
| | ACATCTGTGC CCCAGTCCTC TGTTCCCCGG CCCTAGCAAG CCCTTCTGGG TGCTGGTCGT | 3840 |
| | GGTGGGAGGC GTGCTGGCTT GTTACAGCCT GCTGGTGACA GTTGCCTTCA TTATCTTCTG | 3900 |
| | GGTGAACTGC AGAAATACAG GCCCTTGGCT GAAAAAGGTG CTGAAGTGCA ACACACCTGA | 3960 |
| | CCCCTCCAAA TTCTTCAGCC AGCTGTCCAG CGAGCACGGC GGCGACGTGC AGAAGTGGCT | 4020 |
| | GAGCTCTCCC TTCCCAAGCT CCAGCTTCAG CCCAGGTGGC CTGGCTCCTG AGATCAGCCC | 4080 |
| | TCTCGAAGTC CTGGAACGTG ATAAGGTGAC CCAGCTGCTG CTGCAGCAGG ATAAGGTGCC | 4140 |
| | TGAACCCGCC AGCCTGAGCA GCAATCACAG CCTGACGAGC TGTTTCACCA ACCAGGGATA | 4200 |
| | CTTCTTCTTT CACCTGCCGG ACGCCCTGGA AATCGAGGCC TGTCAGGTGT ACTTCACCTA | 4260 |
| | TGACCCCTAT TCTGAAGAAG ATCCTGATGA GGGCGTGGCA GGAGCTCCAA CCGGCAGCTC | 4320 |
| | TCCCCAGCCC CTGCAGCCCC TGTCTGGCGA GGACGACGCC TACTGCACCT TCCCTTCCAG | 4380 |
| | AGACGACCTG TTACTCTTCT CCCCCAGCCT GCTGGGCGGC CCTTCGCCTC CAAGCACCGC | 4440 |
| | CCCCGGCGGA AGTGGAGCCG GGGAGGAAAG AATGCCCCCC TCCTTGCAGG AGAGAGTGCC | 4500 |
| | TAGAGATTGG GACCCCCAGC CTCTGGGCCC TCCAACCCCT GGCGTGCCTG ACCTCGTGGA | 4560 |
| | CTTCCAACCT CCTCCTGAGC TGGTGCTGAG AGAAGCCGGC GAAGAGGTGC CAGACGCTGG | 4620 |
| | ACCAAGAGAG GGCGTTAGCT TTCCCTGGAG CAGACCTCCC GGCCAGGGCG AGTTTCGGGC | 4680 |
| | CCTGAACGCC AGACTCCCTC TGAACACAGA TGCTTACCTG AGCCTGCAGG AGCTGCAGGG | 4740 |
| | CCAAGACCCT ACCCACCTTG TCGGATCTGG CGAGGGTAGA GGCTCTCTGC TGACCTGTGG | 4800 |
| | CGACGTGGAA GAGAACCCCG GCCCGATGGC CCTGCCTGTG ACCGCCCTAC TCCTGCCTCT | 4860 |
| | GGCCCTGCTG CTCCATGCCG CCAGACCTAA TTTCATGCTG ACTCAGCCTC ACAGCGTGAG | 4920 |
| | CGAGAGCCTG GGAAAGACCG TGACCATCAG CTGCACAGGC AGCAGCGGCA GCATCGCTAG | 4980 |
| | AAAGTTCGTG CAATGGTACC AGCAGAGACC TGGATCTTCT CCTACAACAG TGATCTACGA | 5040 |
| | GAACAACCAG AGACCTAGCG GCGTATCCGA CCGCTTTTCC GGCAGCATCG GGTCCAGCAG | 5100 |
| | CAACAGCGCC AGCCTGACGA TCTCCGGCCT GAAGACAGAG GATGAGGCCG ATTACTACTG | 5160 |
| | CCAGAGCTAC GATAGCTCTA ACGTGGTGTT CGGTGGCGGA ACAAAGGTAA CAGTGCTCGG | 5220 |
| | CGGCGGAGGC TCTGGAGGCG GAGGCAGCGG AGGTGGCGGC TCTGGAGGCG GTGGCAGCCA | 5280 |
| | GGTGCAGCTA CAAGAGAGCG GCGGTGGCCT GGTGAAGCCT GGCGGCAGCC TGCGCCTGAG | 5340 |
| | CTGCGCTGCC AGCGGCTTCA CCTTTAGCTC TTATAGCATG AACTGGGTCC GGCAGGCCCC | 5400 |
| | AGGCAAGGGC CTGGAATGGG TGAGCGGAAT CAACACCGCC GGAGATACCC ACTATCCCGA | 5460 |
| | GAGCGTGAAG GGCAGATTCA CAATTAGCAG AGATAATGCC CGGAACAGCC TGAATCTGCA | 5520 |
| | GATGAACAGC CTGCGGGCTG AAGACACCGC CGTGTACTAT TGCGTGCGGG AACGGGTGGA | 5580 |
| | GAGGGAGTAC AGTGGCTACG ACGCCTTCGA CATCTGGGGC CAGGGAACCA CCGTGACCGT | 5640 |
| | GTCTGCCATC GAAGTGATGT ATCCTCCTCC CTACTTGGAT AACGAAAAAA GCAACGGCAC | 5700 |
| | CATTATCCAC GTAAAGGGGA AGCACCTGTG TCCCAGTCCC CTGTTCCCAG GCCCTTCTAA | 5760 |
| | GCCTTTCTGG GTGCTGGTCG TGGTGGGCGG GGTGCTGGCC TGCTACTCCC TACTGGTGAC | 5820 |
| | CGTGGCCTTT ATCATCTTTT GGGTGGAGCG AACTATGCCA AGAATCCCCA CACTGAAAAA | 5880 |
| | CCTGGAGGAC CTGGTGACAG AGTACCACGG CAATTTCAGC GCCTGGTCCG GCGTGTCTAA | 5940 |
| | GGGACTGGCC GAGAGCCTTC AACCAGACTA CAGCGAGAGA CTGTGCCTGG TGTCTGAGAT | 6000 |
| | CCCCCCTAAG GGCGGAGCCC TGGGCGAAGG ACCCGGCGCC TCTCCCTGCA ATCAGCACAG | 6060 |
| | TCCATACTGG GCCCCTCCGT GCTACACCCT GAAGCCTGAA ACCTGAGAAT TCGATATCAA | 6120 |
| | GCTTATCGGT AATCAACCTC TGGATTACAA AATTTGTGAA AGATTGACTG GTATTCTTAA | 6180 |
| | CTATGTTGCT CCTTTTACGC TATGTGGATA CGCTGCTTTA ATGCCTTTGT ATCATGCTAT | 6240 |
| | TGCTTCCCGT ATGGCTTTCA TTTTCTCCTC CTTGTATAAA TCCTGGTTGC TGTCTCTTTA | 6300 |
| | TGAGGAGTTG TGGCCCGTTG TCAGGCAACG TGGCGTGGTG TGCACTGTGT TTGCTGACGC | 6360 |
| | AACCCCCACT GGTTGGGGCA TTGCCACCAC CTGTCAGCTC CTTTCCGGGA CTTTCGCTTT | 6420 |
| | CCCCCTCCCT ATTGCCACGG CGGAACTCAT CGCCGCCTGC CTTGCCCGCT GCTGGACAGG | 6480 |
| | GGCTCGGCTG TTGGGCACTG ACAATTCCGT GGTGTTGTCG GGGAAATCAT CGTCCTTTCC | 6540 |
| | TTGGCTGCTC GCCTGTGTTG CCACCTGGAT TCTGCGCGGG ACGTCCTTCT GCTACGTCCC | 6600 |
| | TTCGGCCCTC AATCCAGCGG ACCTTCCTTC CCGCGGCCTG CTGCCGGCTC TGCGGCCTCT | 6660 |
| | TCCGCGTCTT CGCCTTCGCC CTCAGACGAG TCGGATCTCC CTTTGGGCCG CCTCCCCGCA | 6720 |
| | TCGATACCGT CGACCTCGAG ACCTAGAAAA ACATGGAGCA ATCACAAGTA GCAATACAGC | 6780 |
| | AGCTACCAAT GCTGATTGTG CCTGGCTAGA AGCACAAGAG GAGGAGGAGG TGGGTTTTCC | 6840 |
| | AGTCACACCT CAGGTACCTT TAAGACCAAT GACTTACAAG GCAGCTGTAG ATCTTAGCCA | 6900 |
| | CTTTTTAAAA GAAAAGGGGG GACTGGAAGG GCTAATTCAC TCCCAACGAA GACAAGATAT | 6960 |
| | CCTTGATCTG TGGASCTACC ACACACAAGG CTACTTCCCT GATTGGCAGA ACTACACACC | 7020 |
| | AGGGCCAGGG ATCAGATATC CACTGACCTT TGGATGGTGC TACAAGCTAG TACCAGTTGA | 7080 |
| | GCAAGAGAAG GTAGAAGAAG CCAATGAAGG AGAGAACACC CGCTTGTTAC ACCCTGTGAG | 7140 |
| | CCTGCATGGG ATGGATGACC CGGAGAGAGA AGTATTAGAG TGGAGGTTTG ACAGCCGCCT | 7200 |
| | AGCATTTCAT CACATGGCCC GAGAGCTGCA TCCGGACTGT ACTGGGTCTC TCTGGTTAGA | 7260 |
| | CCAGATCTGA GCCTGGGAGC TCTCTGGCTA ACTAGGGAAC CCACTGCTTA AGCCTCAATA | 7320 |
| | AAGCTTGCCT TGAGTGCTTC AAGTAGTGTG TGCCCGTCTG TTGTGTGACT CTGGTAACTA | 7380 |
| | GAGATCCCTC AGACCCTTTT AGTCAGTGTG GAAAATCTCT AGCAGGGCCC GTTTAAACCC | 7440 |
| | GCTGATCAGC CTCGACTGTG CCTTCTAGTT GCCAGCCATC TGTTGTTTGC CCCTCCCCCG | 7500 |
| | TGCCTTCCTT GACCCTGGAA GGTGCCACTC CCACTGTCCT TTCCTAATAA AATGAGGAAA | 7560 |
| | TTGCATCGCA TTGTCTGAGT AGGTGTCATT CTATTCTGGG GGGTGGGGTG GGGCAGGACA | 7620 |
| | GCAAGGGGGA GGATTGGGAA GACAATAGCA GGCATGCTGG GGATGCGGTG GGCTCTATGG | 7680 |
| | CTTCTGAGGC GGAAAGAACC AGCTGGGGCT CTAGGGGGTA TCCCCACGCG CCCTGTAGCG | 7740 |
| | GCGCATTAAG CGCGGCGGGT GTGGTGGTTA CGCGCAGCGT GACCGCTACA CTTGCCAGCG | 7800 |
| | CCCTAGCGCC CGCTCCTTTC GCTTTCTTCC CTTCCTTTCT CGCCACGTTC GCCGGCTTTC | 7860 |
| | CCCGTCAAGC TCTAAATCGG GGGCTCCCTT TAGGGTTCCG ATTTAGTGCT TTACGGCACC | 7920 |
| | TCGACCCCAA AAAACTTGAT TAGGGTGATG GTTCACGTAG TGGGCCATCG CCCTGATAGA | 7980 |
| | CGGTTTTTCG CCCTETGACG TTGGAGTCCA CGTTCTTTAA TAGTGGACTC TTGTTCCAAA | 8040 |
| | CTGGAACAAC ACTCAACCCT ATCTCGGTCT ATTCTTTTGA TTTATAAGGG ATTTTGCCGA | 8100 |
| | TTTCGGCCTA TTGGETAAAA AATGAGCTGA TTTAACAAAA ATTTAACGCG AATTAATTCT | 8160 |
| | GTGGAATGTG TGTCAGTTAG GGTGTGGAAA GTCCCCAGGC TCCCCAGCAG GCAGAAGTAT | 8220 |
| | GCAAAGCATG CATCTCAATT AGTCAGCAAC CAGGTGTGGA AAGTCCCCAG GCTCCCCAGC | 8280 |
| | AGGCAGAAGT ATGCAAAGCA TGCATCTCAA TTAGTCAGCA ACCATAGTCC CGCCCCTAAC | 8340 |
| | TCCGCCCATC CCGCCCCTAA CTCCGCCCAG TTCCGCCCAT TCTCCGCCCC ATGGCTGACT | 8400 |
| | AATTTTTTTT ATTTATGCAG AGGCCGAGGC CGCCTCTGCC TCTGAGCTAT TCCAGAAGTA | 8460 |
| | GTGAGGAGGC TTTTTTGGAG GCCTAGGCTT TTGCAAAAAG CTCCCGGGAG CTTGTATATC | 8520 |
| | CATTTTCGGA TCTGATCAGC ACGTGTTGAC AATTAATCAT CGGCATAGTA TATCGGCATA | 8580 |
| | GTATAATACG ACAAGGTGAG GAACTAAACC ATGGCCAAGT TGACCAGTGC CGTTCCGGTG | 8640 |
| | CTCACCGCGC GCGACGTCGC CGGAGCGGTC GAGTTCTGGA CCGACCGGCT CGGGTTCTCC | 8700 |
| | CGGGACTTCG TGGAGGACGA CTTCGCCGGT GTGGTCCGGG ACGACGTGAC CCTGTTCATC | 8760 |
| | AGCGCGGTCC AGGACCAGGT GGTGCCGGAC AACACCCTGG CCTGGGTGTG GGTGCGCGGC | 8820 |
| | CTGGACGAGC TGTACGCCGA GTGGTCGGAG GTCGTGTCCA CGAACTTCCG GGACGCCTCC | 8880 |
| | GGGCCGGCCA TGACCGAGAT CGGCGAGCAG CCGTGGGGGC GGGAGTTCGC CCTGCGCGAC | 8940 |
| | CCGGCCGGCA ACTGCGTGCA CTTCGTGGCC GAGGAGCAGG ACTGACACGT GCTACGAGAT | 9000 |
| | TTCGATTCCA CCGCCGCCTT CTATGAAAGG TTGGGCTTCG GAATCGTTTT CCGGGACGCC | 9060 |
| | GGCTGGATGA TCCTCCAGCG CGGGGATCTC ATGCTGGAGT TCTTCGCCCA CCCCAACTTG | 9120 |
| | TTTATTGCAG CTTATAATGG TTACAAATAA AGCAATAGCA TCACAAATTT CACAAATAAA | 9180 |
| | GCATTTTTTT CACTGCATTC TAGTTGTGGT TTGTCCAAAC TCATCAATGT ATCTTATCAT | 9240 |
| | GTCTGTATAC CGTCGACCTC TAGCTAGAGC TTGGCGTAAT CATGGTCATA GCTGTTTCCT | 9300 |
| | GTGTGAAATT GTTATCCGCT CACAATTCCA CACAACATAC GAGCCGGAAG CATAAAGTGT | 9360 |
| | AAAGCCTGGG GTGCCTAATG AGTGAGCTAA CTCACATTAA TTGCGTTGCG CTCACTGCCC | 9420 |
| | GCTTTCCAGT CGGGAAACCT GTCGTGCCAG CTGCATTAAT GAATCGGCCA ACGCGCGGGG | 9480 |
| | AGAGGCGGTT TGCGTATTGG GCGCTCTTCC GCTTCCTCGC TCACTGACTC GCTGCGCTCG | 9540 |
| | GTCGTTCGGC TGCGGCGAGC GGTATCAGCT CACTCAAAGG CGGTAATACG GTTATCCACA | 9600 |
| | GAATCAGGGG ATAACGCAGG AAAGAACATG TGAGCAAAAG GCCAGCAAAA GGCCAGGAAC | 9660 |
| | CGTAAAAAGG CCGCGTTGCT GGCGTTTTTC CATAGGCTCC GCCCCCCTGA CGAGCATCAC | 9720 |
| | AAAAATCGAC GCTCAAGTCA GAGGTGGCGA AACCCGACAG GACTATAAAG ATACCAGGCG | 9780 |
| | TTTCCCCCTG GAAGCTCCCT CGTGCGCTCT CCTGTTCCGA CCCTGCCGCT TACCGGATAC | 9840 |
| | CTGTCCGCCT TTCTCCCTTC GGGAAGCGTG GCGCTTTCTC ATAGCTCACG CTGTAGGTAT | 9900 |
| | CTCAGTTCGG TGTAGGTCGT TCGCTCCAAG CTGGGCTGTG TGCACGAACC CCCCGTTCAG | 9960 |
| | CCCGACCGCT GCGCCTTATC CGGTAACTAT CGTCTTGAGT CCAACCCGGT AAGACACGAC | 10020 |
| | TTATCGCCAC TGGCAGCAGC CACTGGTAAC AGGATTAGCA GAGCGAGGTA TGTAGGCGGT | 10080 |
| | GCTACAGAGT TCTTGAAGTG GTGGCCTAAC TACGGCTACA CTAGAAGAAC AGTATTTGGT | 10140 |
| | ATCTGCGCTC TGCTGAAGCC AGTTACCTTC GGAAAAAGAG TTGGTAGCTC TTGATCCGGC | 10200 |
| | AAACAAACCA CCGCTGGTAG CGGTGGTTTT TTTGTTTGCA AGCAGCAGAT TACGCGCAGA | 10260 |
| | AAAAAAGGAT CTCAAGAAGA TCCTTTGATC TTTTCTACGG GGTCTGACGC TCAGTGGAAC | 10320 |
| | GAAAACTCAC GTTAAGGGAT TTTGGTCATG AGATTATCAA AAAGGATCTT CACCTAGATC | 10380 |
| | CTTTTAAATT AAAAATGAAG TTTTAAATCA ATCTAAAGTA TATATGAGTA AACTTGGTCT | 10440 |
| | GACAGTTACC AATGCTTAAT CAGTGAGGCA CCTATCTCAG CGATCTGTCT ATTTCGTTCA | 10500 |
| | TCCATAGTTG CCTGACTCCC CGTCGTGTAG ATAACTACGA TACGGGAGGG CTTACCATCT | 10560 |
| | GGCCCCAGTG CTGCAATGAT ACCGCGAGAC CCACGCTCAC CGGCTCCAGA TTTATCAGCA | 10620 |
| | ATAAACCAGC CAGCCGGAAG GGCCGAGCGC AGAAGTGGTC CTGCAACTTT ATCCGCCTCC | 10680 |
| | ATCCAGTCTA TTAATTGTTG CCGGGAAGCT AGAGTAAGTA GTTCGCCAGT TAATAGTTTG | 10740 |
| | CGCAACGTTG TTGCCATTGC TACAGGCATC GTGGTGTCAC GCTCGTCGTT TGGTATGGCT | 10800 |
| | TCATTCAGCT CCGGETCCCA ACGATCAAGG CGAGTTACAT GATCCCCCAT GTTGTGCAAA | 10860 |
| | AAAGCGGTTA GCTCCTTCGG TCCTCCGATC GTTGTCAGAA GTAAGTTGGC CGCAGTGTTA | 10920 |
| | TCACTCATGG TTATGGCAGC ACTGCATAAT TCTCTTACTG TCATGCCATC CGTAAGATGC | 10980 |
| | TTTTCTGTGA CTGGEGAGTA CTCAACCAAG TCATTCTGAG AATAGTGTAT GCGGCGACCG | 11040 |
| | AGTTGCTCTT GCCCGGCGTC AATACGGGAT AATACCGCGC CACATAGCAG AACTTTAAAA | 11100 |
| | GTGCTCATCA TTGGAAAACG TTCTTCGGGG CGAAAACTCT CAAGGATCTT ACCGCTGTTG | 11160 |
| | AGATCCAGTT CGATGTAACC CACTCGTGCA CCCAACTGAT CTTCAGCATC TTTTACTTTC | 11220 |
| | ACCAGCGTTT CTGGGTGAGC AAAAACAGGA AGGCAAAATG CCGCAAAAAA GGGAATAAGG | 11280 |
| | GCGACACGGA AATGETGAAT ACTCATACTC TTCCTTTTTC AATATTATTG AAGCATTTAT | 11340 |
| | CAGGGTTATT GTCTCATGAG CGGATACATA TTTGAATGTA TTTAGAAAAA TAAACAAATA | 11400 |
| | GGGGTTCCGC GCACATTTCC CCGAAAAGTG CCACCTGAC | 11439 |
| SEQ ID NO: 622 | GTCGACGGAT CGGGAGATCT CCCGATCCCC TATGGTGCAC TCTCAGTACA ATCTGCTCTG | 60 |
| pLentiCas9-EGFP | ATGCCGCATA GTTAAGCCAG TATCTGCTCC CTGCTTGTGT GTTGGAGGTC GCTGAGTAGT | 120 |
| vector | GCGCGAGCAA AATTTAAGCT ACAACAAGGC AAGGCTTGAC CGACAATTGC ATGAAGAATC | 180 |
| biepitope CCR | TGCTTAGGGT TAGGCGTTTT GCGCTGCTTC GCGATGTACG GGCCAGATAT ACGCGTTGAC | 240 |
| SP-(38A1 scFv)- | ATTGATTATT GACTAGTTAT TAATAGTAAT CAATTACGGG GTCATTAGTT CATAGCCCAT | 300 |
| (CD28 hinge and | ATATGGAGTT CCGCGTTACA TAACTTACGG TAAATGGCCC GCCTGGCTGA CCGCCCAACG | 360 |
| transmembrane)- | ACCCCCGCCC ATTGACGTCA ATAATGACGT ATGTTCCCAT AGTAACGCCA ATAGGGACTT | 420 |
| (IL-18R1 | TCCATTGACG TCAATGGGTG GAGTATTTAC GGTAAACTGC CCACTTGGCA GTACATCAAG | 480 |
| intracellular)- | TGTATCATAT GCCAAGTACG CCCCCTATTG ACGTCAATGA CGGTAAATGG CCCGCCTGGC | 540 |
| T2A-SP-(19H9 | ATTATGCCCA GTACATGACC TTATGGGACT TTCCTACTTG GCAGTACATC TACGTATTAG | 600 |
| SCEV)-(CD28 | TCATCGCTAT TACCATGGTG ATGCGGTTTT GGCAGTACAT CAATGGGCGT GGATAGCGGT | 660 |
| hinge and | TTGACTCACG GGGASTTCCA AGTCTCCACC CCATTGACGT CAATGGGAGT TTGTTTTGGC | 720 |
| transmembrane)- | ACCAAAATCA ACGGGACTTT CCAAAATGTC GTAACAACTC CGCCCCATTG ACGCAAATGG | 780 |
| (IL-18RAP | GCGGTAGGCG TGTACGGTGG GAGGTCTATA TAAGCAGCGC GTTTTGCCTG TACTGGGTCT | 840 |
| intracellular) | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TGCCCGTCAC CGCTCTGCTG CTGCCTCTGG CCCTGCTGCT | 2940 |
| | GCATGCCGCC AGACCAAGCT ACGTGCTGAC ACAGCCACCT AGCGTGTCCG TGGCCCCTGG | 3000 |
| | CCAGACAGCC AGAATCACCT GTGGCGGCAA CAATATCGGA AGAAAGATCG TGCACTGGTA | 3060 |
| | CCAGCAAAGA CCTGGGCAGG CTCCCGTGCT GGTTATCTAC TACGACACCG ATCGGCCGGC | 3120 |
| | CGGCATCCCT GAAAGATTCA GTGGGAGCAA TTCCGGAAAC ATGGCCACAC TGACGATCTC | 3180 |
| | CACAGTTGGC GCCGGCGACG AAGCCGACTA CTACTGCCAG GTGTGGGACA CTGGCAGTGA | 3240 |
| | TCACGTGGTG TTTGGCGGTG GAACAAAGCT GACCGTGCTG GGCGGCGGTG GCTCCGGCGG | 3300 |
| | CGGCGGGAGC GGCGGTGGGG GCTCTGGAGG AGGAGGCAGT GAGGTGCAGC TGGTTGAGTC | 3360 |
| | TGGCGGCGGC CTCGTGCAAC CTGGCGGCTC CCTGCGGCTG AGCTGCGCCG CTAGCGGCTT | 3420 |
| | TACCTTCAGC AATTACGCCA TGAGCTGGGT CAGACAGGCC CCTGGCAAGG GTCTGGAATG | 3480 |
| | GGTGAGTACC ATCAGCGGCA GCGGTGGCAC CACATACTAC GCCGACAGCG TGAAAGGCAG | 3540 |
| | ATTCACCATC TCCAGAGACA ACAGTAAGAA CACCCTGTAC CTGCAGATGA ACTCCCTGCG | 3600 |
| | GGTCGAGGAC ACCGCTGTGT ACTACTGCGC CAAGGATTGG TTCAGAAGCA GTTCTCCTGA | 3660 |
| | CGCCTTCGAC ATCTGGGGCC AGGGCACAAC AGTGACCGTC TCTGCTATCG AGGTGATGTA | 3720 |
| | TCCTCCTCCA TACCTGGACA ACGAGAAGAG CAATGGTACA ATCATCCACG TGAAAGGAAA | 3780 |
| | GCACCTCTGC CCCAGCCCCC TGTTCCCCGG ACCCAGCAAG CCTTTCTGGG TGCTGGTGGT | 3840 |
| | GGTCGGTGGC GTGCTGGCTT GTTACAGCCT GTTAGTGACC GTGGCCTTTA TCATCTTTTG | 3900 |
| | GGTTTACAGG GTGGACCTGG TGCTGTTCTA CCGGCACTTG ACAAGAAGAG ACGAGACACT | 3960 |
| | GACAGACGGC AAGACCTACG ATGCCTTCGT GAGCTACCTG AAGGAATGTA GACCCGAGAA | 4020 |
| | CGGCGAGGAA CACACCTTCG CCGTGGAAAT CCTGCCTCGG GTGCTGGAAA AGCATTTCGG | 4080 |
| | CTATAAGCTG TGTATCTTTG AGCGGGATGT GGTGCCGGGC GGAGCCGTGG TAGACGAGAT | 4140 |
| | CCACAGCCTG ATCGAGAAGA GCAGAAGGCT GATCATCGTG CTGTCTAAGA GCTACATGAG | 4200 |
| | CAACGAGGTG AGATACGAGC TGGAGAGCGG CCTGCACGAG GCCCTGGTGG AAAGAAAGAT | 4260 |
| | CAAGATCATC CTTATCGAGT TCACCCCCGT GACCGACTTC ACATTCCTGC CCCAGTCTCT | 4320 |
| | GAAACTGCTC AAGAGCCACA GAGTGCTGAA ATGGAAGGCC GATAAGAGCC TGTCTTACAA | 4380 |
| | CAGCAGATTC TGGAAGAACC TGCTGTATCT GATGCCTGCT AAGACAGTGA AGCCTGGCAG | 4440 |
| | GGATGAGCCC GAGGTGCTGC CCGTGCTGTC TGAGAGCGGC AGCGGAGAGG GCCGGGGCAG | 4500 |
| | CTTACTGACC TGCGGTGACG TGGAAGAAAA CCCTGGACCT ATGGCCCTGC CAGTGACCGC | 4560 |
| | CCTGCTGCTG CCCCEGGCCC TGCTGCTGCA CGCCGCTCGA CCCAACTTCA TGCTGACCCA | 4620 |
| | GCCTCACAGC GTGTCCGAAA GCTTGGGCAA GACCGTGACT ATATCTTGCA CCGGGTCTTC | 4680 |
| | CGGCAGCATC GCCAGGAAGT TTGTGCAGTG GTATCAGCAG AGACCTGGTA GCAGCCCAAC | 4740 |
| | CACCGTGATC TACGAGAACA ACCAGCGGCC CAGCGGAGTG TCCGACCGGT TCAGCGGCTC | 4800 |
| | TATCGGCTCA TCCAGCAATT CTGCCAGCCT GACCATATCT GGCTTGAAAA CCGAGGACGA | 4860 |
| | AGCAGATTAC TACTGTCAGA GCTATGATAG CAGCAACGTG GTTTTCGGCG GCGGCACCAA | 4920 |
| | GGTGACAGTG CTCGGAGGCG GCGGCTCAGG GGGCGGAGGA TCAGGCGGCG GCGGCAGCGG | 4980 |
| | CGGAGGCGGC AGCCAAGTGC AGCTGCAGGA GAGCGGAGGC GGCCTGGTGA AACCTGGCGG | 5040 |
| | GTCTCTGAGG CTGAGCTGCG CCGCCTCTGG ATTTACCTTC AGCAGCTACA GCATGAACTG | 5100 |
| | GGTCAGACAG GCCCCTGGCA AGGGACTGGA ATGGGTGTCT GGCATCAACA CCGCCGGCGA | 5160 |
| | CACCCACTAC CCCGAATCCG TGAAGGGCAG ATTCACTATC TCCCGCGATA ATGCCAGAAA | 5220 |
| | CTCCCTCAAC CTGCAAATGA ACAGCCTGCG GGCCGAGGAC ACCGCTGTGT ACTATTGCGT | 5280 |
| | GAGAGAGAGA GTTGAACGGG AATACTCCGG CTACGACGCC TTTGACATCT GGGGCCAGGG | 5340 |
| | AACCACAGTG ACAGTGTCTG CCATCGAAGT GATGTACCCC CCACCTTATC TGGATAACGA | 5400 |
| | GAAGAGCAAC GGCACCATCA TTCACGTGAA AGGCAAACAT CTGTGCCCTT CTCCTCTGTT | 5460 |
| | TCCTGGCCCT TCTAAGCCCT TCTGGGTCCT GGTGGTGGTG GGCGGCGTGC TAGCCTGCTA | 5520 |
| | CAGCCTGCTG GTCACCGTTG CCTTCATCAT CTTTTGGGTG AGCGCCTTGC TGTATAGACA | 5580 |
| | CTGGATCGAG ATTGTGCTGC TGTACCGGAC ATACCAGAGC AAGGACCAGA CCCTGGGCGA | 5640 |
| | CAAGAAGGAC TTCGACGCCT TCGTGTCCTA CGCCAAGTGG AGCAGCTTCC CAAGCGAGGC | 5700 |
| | CACCAGCTCC CTGAGCGAAG AGCACCTGGC TCTGAGCCTG TTCCCCGATG TGCTGGAAAA | 5760 |
| | CAAGTACGGC TACTCACTCT GCCTGCTGGA AAGAGATGTG GCACCTGGCG GCGTGTACGC | 5820 |
| | AGAGGATATC GTGTCCATCA TTAAGCGAAG CCGGAGAGGC ATCTTCATCC TGTCCCCTAA | 5880 |
| | TTACGTGAAC GGCCCCAGCA TCTTCGAGCT GCAGGCCGCT GTCAACCTGG CCCTGGACGA | 5940 |
| | CCAGACCCTG AAGCTGATCC TGATCAAATT CTGCTACTTC CAGGAGCCTG AGAGCCTGCC | 6000 |
| | TCACCTTGTC AAGAAAGCCC TGAGAGTGCT GCCAACCGTG ACATGGCGGG GGCTGAAGAG | 6060 |
| | CGTTCCTCCT AACAGCCGGT TTTGGGCCAA AATGCGGTAC CACATGCCCG TGAAGAACAG | 6120 |
| | CCAGGGCTTC ACCTGGAACC AGCTGAGAAT CACCAGCAGA ATCTTCCAGT GGAAGGGCCT | 6180 |
| | GAGCCGGACC GAGACAACCG GCCGGAGCAG CCAGCCAAAG GAATGGTGAT AAGAATTCGA | 6240 |
| | TATCAAGCTT ATCGGTAATC AACCTCTGGA TTACAAAATT TGTGAAAGAT TGACTGGTAT | 6300 |
| | TCTTAACTAT GTTGCTCCTT TTACGCTATG TGGATACGCT GCTTTAATGC CTTTGTATCA | 6360 |
| | TGCTATTGCT TCCCGTATGG CTTTCATTTT CTCCTCCTTG TATAAATCCT GGTTGCTGTC | 6420 |
| | TCTTTATGAG GAGTTGTGGC CCGTTGTCAG GCAACGTGGC GTGGTGTGCA CTGTGTTTGC | 6480 |
| | TGACGCAACC CCCACTGGTT GGGGCATTGC CACCACCTGT CAGCTCCTTT CCGGGACTTT | 6540 |
| | CGCTTTCCCC CTCCCTATTG CCACGGCGGA ACTCATCGCC GCCTGCCTTG CCCGCTGCTG | 6600 |
| | GACAGGGGCT CGGCTGTTGG GCACTGACAA TTCCGTGGTG TTGTCGGGGA AATCATCGTC | 6660 |
| | CTTTCCTTGG CTGCTCGCCT GTGTTGCCAC CTGGATTCTG CGCGGGACGT CCTTCTGCTA | 6720 |
| | CGTCCCTTCG GCCCTCAATC CAGCGGACCT TCCTTCCCGC GGCCTGCTGC CGGCTCTGCG | 6780 |
| | GCCTCTTCCG CGTCTTCGCC TTCGCCCTCA GACGAGTCGG ATCTCCCTTT GGGCCGCCTC | 6840 |
| | CCCGCATCGA TACCGTCGAC CTCGAGACCT AGAAAAACAT GGAGCAATCA CAAGTAGCAA | 6900 |
| | TACAGCAGCT ACCAATGCTG ATTGTGCCTG GCTAGAAGCA CAAGAGGAGG AGGAGGTGGG | 6960 |
| | TTTTCCAGTC ACACCTCAGG TACCTTTAAG ACCAATGACT TACAAGGCAG CTGTAGATCT | 7020 |
| | TAGCCACTTT TTAAAAGAAA AGGGGGGACT GGAAGGGCTA ATTCACTCCC AACGAAGACA | 7080 |
| | AGATATCCTT GATCTGTGGA TCTACCACAC ACAAGGCTAC TTCCCTGATT GGCAGAACTA | 7140 |
| | CACACCAGGG CCAGGGATCA GATATCCACT GACCTTTGGA TGGTGCTACA AGCTAGTACC | 7200 |
| | AGTTGAGCAA GAGAAGGTAG AAGAAGCCAA TGAAGGAGAG AACACCCGCT TGTTACACCC | 7260 |
| | TGTGAGCCTG CATGGGATGG ATGACCCGGA GAGAGAAGTA TTAGAGTGGA GGTTTGACAG | 7320 |
| | CCGCCTAGCA TTTCATCACA TGGCCCGAGA GCTGCATCCG GACTGTACTG GGTCTCTCTG | 7380 |
| | GTTAGACCAG ATCTGAGCCT GGGAGCTCTC TGGCTAACTA GGGAACCCAC TGCTTAAGCC | 7440 |
| | TCAATAAAGC TTGCCTTGAG TGCTTCAAGT AGTGTGTGCC CGTCTGTTGT GTGACTCTGG | 7500 |
| | TAACTAGAGA TCCCTCAGAC CCTTTTAGTC AGTGTGGAAA ATCTCTAGCA GGGCCCGTTT | 7560 |
| | AAACCCGCTG ATCAGCCTCG ACTGTGCCTT CTAGTTGCCA GCCATCTGTT GTTTGCCCCT | 7620 |
| | CCCCCGTGCC TTCCTTGACC CTGGAAGGTG CCACTCCCAC TGTCCTTTCC TAATAAAATG | 7680 |
| | AGGAAATTGC ATCGCATTGT CTGAGTAGGT GTCATTCTAT TCTGGGGGGT GGGGTGGGGC | 7740 |
| | AGGACAGCAA GGGGGAGGAT TGGGAAGACA ATAGCAGGCA TGCTGGGGAT GCGGTGGGCT | 7800 |
| | CTATGGCTTC TGAGGCGGAA AGAACCAGCT GGGGCTCTAG GGGGTATCCC CACGCGCCCT | 7860 |
| | GTAGCGGCGC ATTAAGCGCG GCGGGTGTGG TGGTTACGCG CAGCGTGACC GCTACACTTG | 7920 |
| | CCAGCGCCCT AGCGCCCGCT CCTTTCGCTT TCTTCCCTTC CTTTCTCGCC ACGTTCGCCG | 7980 |
| | GCTTTCCCCG TCAAGCTCTA AATCGGGGGC TCCCTTTAGG GTTCCGATTT AGTGCTTTAC | 8040 |
| | GGCACCTCGA CCCCAAAAAA CTTGATTAGG GTGATGGTTC ACGTAGTGGG CCATCGCCCT | 8100 |
| | GATAGACGGT TTTTCGCCCT TTGACGTTGG AGTCCACGTT CTTTAATAGT GGACTCTTGT | 8160 |
| | TCCAAACTGG AACAACACTC AACCCTATCT CGGTCTATTC TTTTGATTTA TAAGGGATTT | 8220 |
| | TGCCGATTTC GGCCTATTGG TTAAAAAATG AGCTGATTTA ACAAAAATTT AACGCGAATT | 8280 |
| | AATTCTGTGG AATGTGTGTC AGTTAGGGTG TGGAAAGTCC CCAGGCTCCC CAGCAGGCAG | 8340 |
| | AAGTATGCAA AGCATGCATC TCAATTAGTC AGCAACCAGG TGTGGAAAGT CCCCAGGCTC | 8400 |
| | CCCAGCAGGC AGAAGTATGC AAAGCATGCA TCTCAATTAG TCAGCAACCA TAGTCCCGCC | 8460 |
| | CCTAACTCCG CCCATCCCGC CCCTAACTCC GCCCAGTTCC GCCCATTCTC CGCCCCATGG | 8520 |
| | CTGACTAATT TTTTTTATTT ATGCAGAGGC CGAGGCCGCC TCTGCCTCTG AGCTATTCCA | 8580 |
| | GAAGTAGTGA GGAGGCTTTT TTGGAGGCCT AGGCTTTTGC AAAAAGCTCC CGGGAGCTTG | 8640 |
| | TATATCCATT TTCGGATCTG ATCAGCACGT GTTGACAATT AATCATCGGC ATAGTATATC | 8700 |
| | GGCATAGTAT AATACGACAA GGTGAGGAAC TAAACCATGG CCAAGTTGAC CAGTGCCGTT | 8760 |
| | CCGGTGCTCA CCGCGCGCGA CGTCGCCGGA GCGGTCGAGT TCTGGACCGA CCGGCTCGGG | 8820 |
| | TTCTCCCGGG ACTTCGTGGA GGACGACTTC GCCGGTGTGG TCCGGGACGA CGTGACCCTG | 8880 |
| | TTCATCAGCG CGGTCCAGGA CCAGGTGGTG CCGGACAACA CCCTGGCCTG GGTGTGGGTG | 8940 |
| | CGCGGCCTGG ACGAGCTGTA CGCCGAGTGG TCGGAGGTCG TGTCCACGAA CTTCCGGGAC | 9000 |
| | GCCTCCGGGC CGGCCATGAC CGAGATCGGC GAGCAGCCGT GGGGGGGGGA GTTCGCCCTG | 9060 |
| | CGCGACCCGG CCGGCAACTG CGTGCACTTC GTGGCCGAGG AGCAGGACTG ACACGTGCTA | 9120 |
| | CGAGATTTCG ATTCCACCGC CGCCTTCTAT GAAAGGTTGG GCTTCGGAAT CGTTTTCCGG | 9180 |
| | GACGCCGGCT GGATGATCCT CCAGCGCGGG GATCTCATGC TGGAGTTCTT CGCCCACCCC | 9240 |
| | AACTTGTTTA TTGCAGCTTA TAATGGTTAC AAATAAAGCA ATAGCATCAC AAATTTCACA | 9300 |
| | AATAAAGCAT TTTTTTCACT GCATTCTAGT TGTGGTTTGT CCAAACTCAT CAATGTATCT | 9360 |
| | TATCATGTCT GTATACCGTC GACCTCTAGC TAGAGCTTGG CGTAATCATG GTCATAGCTG | 9420 |
| | TTTCCTGTGT GAAASTGTTA TCCGCTCACA ATTCCACACA ACATACGAGC CGGAAGCATA | 9480 |
| | AAGTGTAAAG CCTGGGGTGC CTAATGAGTG AGCTAACTCA CATTAATTGC GTTGCGCTCA | 9540 |
| | CTGCCCGCTT TCCAGTCGGG AAACCTGTCG TGCCAGCTGC ATTAATGAAT CGGCCAACGC | 9600 |
| | GCGGGGAGAG GCGGTTTGCG TATTGGGCGC TCTTCCGCTT CCTCGCTCAC TGACTCGCTG | 9660 |
| | CGCTCGGTCG TTCGGCTGCG GCGAGCGGTA TCAGCTCACT CAAAGGCGGT AATACGGTTA | 9720 |
| | TCCACAGAAT CAGGGGATAA CGCAGGAAAG AACATGTGAG CAAAAGGCCA GCAAAAGGCC | 9780 |
| | AGGAACCGTA AAAAGGCCGC GTTGCTGGCG TTTTTCCATA GGCTCCGCCC CCCTGACGAG | 9840 |
| | CATCACAAAA ATCGACGCTC AAGTCAGAGG TGGCGAAACC CGACAGGACT ATAAAGATAC | 9900 |
| | CAGGCGTTTC CCCCTGGAAG CTCCCTCGTG CGCTCTCCTG TTCCGACCCT GCCGCTTACC | 9960 |
| | GGATACCTGT CCGCCTTTCT CCCTTCGGGA AGCGTGGCGC TTTCTCATAG CTCACGCTGT | 10020 |
| | AGGTATCTCA GTTCGGTGTA GGTCGTTCGC TCCAAGCTGG GCTGTGTGCA CGAACCCCCC | 10080 |
| | GTTCAGCCCG ACCGCTGCGC CTTATCCGGT AACTATCGTC TTGAGTCCAA CCCGGTAAGA | 10140 |
| | CACGACTTAT CGCCACTGGC AGCAGCCACT GGTAACAGGA TTAGCAGAGC GAGGTATGTA | 10200 |
| | GGCGGTGCTA CAGAGTTCTT GAAGTGGTGG CCTAACTACG GCTACACTAG AAGAACAGTA | 10260 |
| | TTTGGTATCT GCGCTCTGCT GAAGCCAGTT ACCTTCGGAA AAAGAGTTGG TAGCTCTTGA | 10320 |
| | TCCGGCAAAC AAACCACCGC TGGTAGCGGT GGTTTTTTTG TTTGCAAGCA GCAGATTACG | 10380 |
| | CGCAGAAAAA AAGGATCTCA AGAAGATCCT TTGATCTTTT CTACGGGGTC TGACGCTCAG | 10440 |
| | TGGAACGAAA ACTCACGTTA AGGGATTTTG GTCATGAGAT TATCAAAAAG GATCTTCACC | 10500 |
| | TAGATCCTTT TAAASTAAAA ATGAAGTTTT AAATCAATCT AAAGTATATA TGAGTAAACT | 10560 |
| | TGGTCTGACA GTTACCAATG CTTAATCAGT GAGGCACCTA TCTCAGCGAT CTGTCTATTT | 10620 |
| | CGTTCATCCA TAGTTGCCTG ACTCCCCGTC GTGTAGATAA CTACGATACG GGAGGGCTTA | 10680 |
| | CCATCTGGCC CCAGTGCTGC AATGATACCG CGAGACCCAC GCTCACCGGC TCCAGATTTA | 10740 |
| | TCAGCAATAA ACCAGCCAGC CGGAAGGGCC GAGCGCAGAA GTGGTCCTGC AACTTTATCC | 10800 |
| | GCCTCCATCC AGTCTATTAA TTGTTGCCGG GAAGCTAGAG TAAGTAGTTC GCCAGTTAAT | 10860 |
| | AGTTTGCGCA ACGTTGTTGC CATTGCTACA GGCATCGTGG TGTCACGCTC GTCGTTTGGT | 10920 |
| | ATGGCTTCAT TCAGCTCCGG TTCCCAACGA TCAAGGCGAG TTACATGATC CCCCATGTTG | 10980 |
| | TGCAAAAAAG CGGTTAGCTC CTTCGGTCCT CCGATCGTTG TCAGAAGTAA GTTGGCCGCA | 11040 |
| | GTGTTATCAC TCATGGTTAT GGCAGCACTG CATAATTCTC TTACTGTCAT GCCATCCGTA | 11100 |
| | AGATGCTTTT CTGTGACTGG TGAGTACTCA ACCAAGTCAT TCTGAGAATA GTGTATGCGG | 11160 |
| | CGACCGAGTT GCTCTTGCCC GGCGTCAATA CGGGATAATA CCGCGCCACA TAGCAGAACT | 11220 |
| | TTAAAAGTGC TCATCATTGG AAAACGTTCT TCGGGGCGAA AACTCTCAAG GATCTTACCG | 11280 |
| | CTGTTGAGAT CCAGTTCGAT GTAACCCACT CGTGCACCCA ACTGATCTTC AGCATCTTTT | 11340 |
| | ACTTTCACCA GCGTTTCTGG GTGAGCAAAA ACAGGAAGGC AAAATGCCGC AAAAAAGGGA | 11400 |
| | ATAAGGGCGA CACGGAAATG TTGAATACTC ATACTCTTCC TTTTTCAATA TTATTGAAGC | 11460 |
| | ATTTATCAGG GTTATTGTCT CATGAGCGGA TACATATTTG AATGTATTTA GAAAAATAAA | 11520 |
| | CAAATAGGGG TTCCGCGCAC ATTTCCCCGA AAAGTGCCAC CTGAC | 11565 |
| SEQ ID NO: 623 | GTCGACGGAT CGGGAGATCT CCCGATCCCC TATGGTGCAC TCTCAGTACA ATCTGCTCTG | 60 |
| pLentiCas9-EGFP | ATGCCGCATA GTTAAGCCAG TATCTGCTCC CTGCTTGTGT GTTGGAGGTC GCTGAGTAGT | 120 |
| vector | GCGCGAGCAA AATTTAAGCT ACAACAAGGC AAGGCTTGAC CGACAATTGC ATGAAGAATC | 180 |
| biepitope CCR | TGCTTAGGGT TAGGCGTTTT GCGCTGCTTC GCGATGTACG GGCCAGATAT ACGCGTTGAC | 240 |
| SP-(anti-TROP-2 | ATTGATTATT GACTAGTTAT TAATAGTAAT CAATTACGGG GTCATTAGTT CATAGCCCAT | 300 |
| SCEv)-(CD8 | ATATGGAGTT CCGCGTTACA TAACTTACGG TAAATGGCCC GCCTGGCTGA CCGCCCAACG | 360 |
| hinge)-(IL-2RB | ACCCCCGCCC ATTGACGTCA ATAATGACGT ATGTTCCCAT AGTAACGCCA ATAGGGACTT | 420 |
| transmembrane | TCCATTGACG TCAATGGGTG GAGTATTTAC GGTAAACTGC CCACTTGGCA GTACATCAAG | 480 |
| and | TGTATCATAT GCCAAGTACG CCCCCTATTG ACGTCAATGA CGGTAAATGG CCCGCCTGGC | 540 |
| intracellular)- | ATTATGCCCA GTACATGACC TTATGGGACT TTCCTACTTG GCAGTACATC TACGTATTAG | 600 |
| T2A-SP- | TCATCGCTAT TACCATGGTG ATGCGGTTTT GGCAGTACAT CAATGGGCGT GGATAGCGGT | 660 |
| (anti-TROP-2 | TTGACTCACG GGGATTTCCA AGTCTCCACC CCATTGACGT CAATGGGAGT TTGTTTTGGC | 720 |
| SCEV)-(CD8 | ACCAAAATCA ACGGGACTTT CCAAAATGTC GTAACAACTC CGCCCCATTG ACGCAAATGG | 780 |
| hinge)-(IL-2Rγ | GCGGTAGGCG TGTACGGTGG GAGGTCTATA TAAGCAGCGC GTTTTGCCTG TACTGGGTCT | 840 |
| transmembrane | CTCTGGTTAG ACCAGATCTG AGCCTGGGAG CTCTCTGGCT AACTAGGGAA CCCACTGCTT | 900 |
| and | AAGCCTCAAT AAAGCTTGCC TTGAGTGCTT CAAGTAGTGT GTGCCCGTCT GTTGTGTGAC | 960 |
| intracellular) | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCAEGGCCC TGCCCGTGAC AGCCCTGCTG CTGCCTCTGG CCCTGCTGCT | 2940 |
| | GCACGCCGCC CGGCCTGAAA TCGTGCTGAC CCAGAGCCCC GCCACACTGA GCCTGAGCCC | 3000 |
| | TGGCGAACGG GCCACCCTGT CCTGTAGAGC CTCTCAGACC ATCGGCACCA GCATCCACTG | 3060 |
| | GTATCAGCAG AAGCCTGGCC AAGCCCCACG GCTGCTCATC TACTATGCGA GCGAGAGCAT | 3120 |
| | CAGCGGCATC CCCGCCCGGT TCAGCGGCAG CGGCTCCGGC ACAGATTTCA CACTGACTAT | 3180 |
| | CAGCTCCCTG GAACCCGAGG ATTTTGCCGT GTACTACTGC CAGCAGAGCA ATAGCTGGCC | 3240 |
| | CTTCACCTTC GGCCAGGGCA CAAAGCTGGA AATCAAGGGC GGCGGCGGCA GCGGCGGCGG | 3300 |
| | CGGTAGTGGC GGCGGGGGCA GCGGCGGCGG CGGCTCCCAA GTGCAGCTGG TCCAGAGCGG | 3360 |
| | AGCCGAGGTG AAGAAGCCCG GCGCCTCAGT GAAGGTGTCT TGCAAAGCCT CTGGCTACAC | 3420 |
| | CTTCACCTCT TACTGGATCA ACTGGGTCAG ACAGGCTCCG GGCCAGGGCT TGGAGTGGAT | 3480 |
| | GGGAAATATC TACCCTAGCG ACAGCTATTC TAACTACAAT CAAAAGTTTA AGGACAGAGT | 3540 |
| | GACAATGACC AGAGATACCA GCACATCCAC CGTGTACATG GAACTGAGCT CCCTTAGAAG | 3600 |
| | CGAGGACACG GCCGTGTACT ACTGCGCCAG AGGCTCTTCC TTCGACTACT GGGGCCAGGG | 3660 |
| | CACACTGGTG ACCGTGTCTA AGCCTACAAC CACCCCTGCC CCTAGGCCCC CTACCCCTGC | 3720 |
| | CCCTACAATC GCCAGCCAAC CTCTGTCGCT GCGGCCTGAG GCCTGCAGAC CTGCAGCTGG | 3780 |
| | CGGCGCTGTG CACACACGCG GCCTGGACTT TGCCTGTGAC ATCTACATCC CTTGGCTGGG | 3840 |
| | CCACCTGCTG GTTGGCCTGA GCGGAGCCTT CGGCTTCATC ATCCTGGTGT ACCTGCTGAT | 3900 |
| | CAACTGCAGA AACACCGGCC CCTGGCTGAA AAAGGTGCTG AAGTGCAATA CCCCTGACCC | 3960 |
| | TAGCAAGTTC TTTAGCCAGC TGTCCAGCGA GCACGGCGGC GACGTGCAGA AATGGCTGAG | 4020 |
| | CTCTCCTTTT CCATCCAGTT CTTTCAGCCC TGGCGGACTG GCCCCTGAGA TTTCTCCTCT | 4080 |
| | GGAAGTGCTG GAGAGAGACA AGGTGACACA GCTGCTGCTG CAGCAGGACA AGGTGCCCGA | 4140 |
| | GCCCGCCTCC CTGAGCAGCA ACCACTCCCT GACCAGCTGC TTCACCAACC AGGGTTATTT | 4200 |
| | CTTCTTCCAC CTGCCTGACG CCCTGGAAAT CGAGGCCTGC CAGGTGTACT TTACATACGA | 4260 |
| | CCCCTACAGC GAAGAGGATC CGGACGAGGG AGTGGCCGGA GCTCCTACCG GCAGCAGCCC | 4320 |
| | TCAGCCACTG CAGCCCCTTA GTGGCGAGGA TGACGCTTAC TGCACCTTCC CTTCGAGAGA | 4380 |
| | TGACCTGTTG CTGTTCAGCC CCAGCCTCCT GGGTGGCCCT AGCCCTCCAA GCACCGCCCC | 4440 |
| | AGGCGGCTCA GGAGCCGGCG AAGAGCGGAT GCCTCCTTCC CTGCAGGAGA GAGTGCCACG | 4500 |
| | GGACTGGGAC CCCCAGCCCC TGGGCCCTCC AACCCCTGGC GTGCCTGATC TGGTCGACTT | 4560 |
| | TCAGCCTCCT CCAGAGCTGG TCCTGAGAGA AGCCGGGGAA GAGGTGCCCG ACGCCGGACC | 4620 |
| | TAGAGAGGGC GTGAGCTTTC CCTGGAGCAG ACCCCCCGGA CAAGGCGAGT TCAGAGCCCT | 4680 |
| | GAATGCCAGA CTGCCGCTTA ACACCGATGC TTACCTGAGC CTGCAAGAGC TGCAGGGCCA | 4740 |
| | GGATCCTACC CACCEGGTGG GATCCGGGGA AGGCAGAGGC TCTCTGCTGA CCTGTGGCGA | 4800 |
| | CGTCGAGGAA AACCCCGGCC CCATGGCTCT GCCGGTGACC GCCCTGCTGT TACCTCTCGC | 4860 |
| | CCTGCTGCTG CATGCCGCCA GACCCGAGAT TGTGTTGACC CAGAGTCCTG CCACACTGAG | 4920 |
| | CCTGAGCCCA GGAGAGCGGG CTACACTGTC ATGCAGAGCC AGCCAGACCA TCGGCACCAG | 498C |
| | CATCCACTGG TACCAGCAGA AGCCTGGACA GGCTCCTCGG CTGCTGATCT ACTACGCCAG | 5040 |
| | CGAAAGCATC AGCGGAATCC CCGCTAGATT CTCAGGCAGC GGCAGCGGCA CAGACTTCAC | 5100 |
| | CCTGACAATC AGCTCCCTGG AGCCTGAGGA CTTCGCCGTG TACTACTGCC AGCAAAGCAA | 5160 |
| | CAGCTGGCCT TTCACATTCG GACAGGGCAC CAAGCTGGAG ATTAAGGGCG GAGGCGGCTC | 5220 |
| | TGGGGGCGGC GGCAGCGGAG GCGGCGGGTC CGGCGGAGGT GGCTCTCAGG TCCAGCTGGT | 5280 |
| | GCAGAGCGGG GCTGAAGTGA AAAAACCAGG CGCCTCAGTG AAGGTAAGCT GTAAAGCCAG | 5340 |
| | CGGCTACACC TTCACTTCTT ACTGGATCAA CTGGGTGCGG CAGGCTCCTG GACAAGGACT | 5400 |
| | GGAATGGATG GGCAACATCT ACCCCTCTGA TAGCTACAGC AACTACAACC AGAAGTTCAA | 5460 |
| | GGACCGGGTG ACCATGACAA GAGACACCTC CACCTCCACC GTCTACATGG AACTGAGCTC | 5520 |
| | TCTGCGGAGC GAGGATACCG CCGTGTACTA CTGCGCCAGA GGTTCGAGCT TTGACTATTG | 5580 |
| | GGGACAGGGG ACCCTGGTGA CCGTGTCCAA ACCTACAACG ACCCCCGCGC CAAGACCTCC | 5640 |
| | GACACCCGCC CCAACCATCG CTAGCCAGCC TCTGTCTCTG AGACCTGAGG CCTGTCGGCC | 5700 |
| | CGCCGCTGGC GGCGCCGTGC ATACCAGAGG CCTGGACTTC GCCTGCGACA TCTACGTGGT | 5760 |
| | GATCAGCGTG GGCAGTATGG GCCTGATAAT CTCCCTCCTT TGTGTGTACT TCTGGCTGGA | 5820 |
| | AAGAACTATG CCTCGGATCC CTACCCTGAA GAACCTGGAG GATCTGGTGA CCGAGTACCA | 5880 |
| | CGGCAACTTC AGCGCCTGGT CCGGTGTTAG CAAAGGCCTC GCCGAATCTC TTCAGCCTGA | 5940 |
| | CTACTCTGAA AGACTATGCC TGGTCAGCGA AATCCCTCCT AAGGGCGGCG CCCTGGGAGA | 6000 |
| | GGGCCCTGGC GCTTCTCCTT GCAACCAGCA CAGCCCTTAC TGGGCCCCTC CTTGTTACAC | 6060 |
| | CCTGAAGCCT GAGACATGAT AAGAATTCGA TATCAAGCTT ATCGGTAATC AACCTCTGGA | 6120 |
| | TTACAAAATT TGTGAAAGAT TGACTGGTAT TCTTAACTAT GTTGCTCCTT TTACGCTATG | 6180 |
| | TGGATACGCT GCTTTAATGC CTTTGTATCA TGCTATTGCT TCCCGTATGG CTTTCATTTT | 6240 |
| | CTCCTCCTTG TATAAATCCT GGTTGCTGTC TCTTTATGAG GAGTTGTGGC CCGTTGTCAG | 6300 |
| | GCAACGTGGC GTGGTGTGCA CTGTGTTTGC TGACGCAACC CCCACTGGTT GGGGCATTGC | 6360 |
| | CACCACCTGT CAGCTCCTTT CCGGGACTTT CGCTTTCCCC CTCCCTATTG CCACGGCGGA | 6420 |
| | ACTCATCGCC GCCTGCCTTG CCCGCTGCTG GACAGGGGCT CGGCTGTTGG GCACTGACAA | 6480 |
| | TTCCGTGGTG TTGTCGGGGA AATCATCGTC CTTTCCTTGG CTGCTCGCCT GTGTTGCCAC | 6540 |
| | CTGGATTCTG CGCGGGACGT CCTTCTGCTA CGTCCCTTCG GCCCTCAATC CAGCGGACCT | 6600 |
| | TCCTTCCCGC GGCCTGCTGC CGGCTCTGCG GCCTCTTCCG CGTCTTCGCC TTCGCCCTCA | 6660 |
| | GACGAGTCGG ATCTCCCTTT GGGCCGCCTC CCCGCATCGA TACCGTCGAC CTCGAGACCT | 6720 |
| | AGAAAAACAT GGAGCAATCA CAAGTAGCAA TACAGCAGCT ACCAATGCTG ATTGTGCCTG | 6780 |
| | GCTAGAAGCA CAAGAGGAGG AGGAGGTGGG TTTTCCAGTC ACACCTCAGG TACCTTTAAG | 6840 |
| | ACCAATGACT TACAAGGCAG CTGTAGATCT TAGCCACTTT TTAAAAGAAA AGGGGGGACT | 6900 |
| | GGAAGGGCTA ATTCACTCCC AACGAAGACA AGATATCCTT GATCTGTGGA TCTACCACAC | 6960 |
| | ACAAGGCTAC TTCCCTGATT GGCAGAACTA CACACCAGGG CCAGGGATCA GATATCCACT | 7020 |
| | GACCTTTGGA TGGTGCTACA AGCTAGTACC AGTTGAGCAA GAGAAGGTAG AAGAAGCCAA | 7080 |
| | TGAAGGAGAG AACACCCGCT TGTTACACCC TGTGAGCCTG CATGGGATGG ATGACCCGGA | 7140 |
| | GAGAGAAGTA TTAGAGTGGA GGTTTGACAG CCGCCTAGCA TTTCATCACA TGGCCCGAGA | 7200 |
| | GCTGCATCCG GACTGTACTG GGTCTCTCTG GTTAGACCAG ATCTGAGCCT GGGAGCTCTC | 7260 |
| | TGGCTAACTA GGGAACCCAC TGCTTAAGCC TCAATAAAGC TTGCCTTGAG TGCTTCAAGT | 7320 |
| | AGTGTGTGCC CGTCTGTTGT GTGACTCTGG TAACTAGAGA TCCCTCAGAC CCTTTTAGTC | 7380 |
| | AGTGTGGAAA ATCTCTAGCA GGGCCCGTTT AAACCCGCTG ATCAGCCTCG ACTGTGCCTT | 7440 |
| | CTAGTTGCCA GCCATCTGTT GTTTGCCCCT CCCCCGTGCC TTCCTTGACC CTGGAAGGTG | 7500 |
| | CCACTCCCAC TGTCCTTTCC TAATAAAATG AGGAAATTGC ATCGCATTGT CTGAGTAGGT | 7560 |
| | GTCATTCTAT TCTGGGGGGT GGGGTGGGGC AGGACAGCAA GGGGGAGGAT TGGGAAGACA | 7620 |
| | ATAGCAGGCA TGCTGGGGAT GCGGTGGGCT CTATGGCTTC TGAGGCGGAA AGAACCAGCT | 7680 |
| | GGGGCTCTAG GGGGTATCCC CACGCGCCCT GTAGCGGCGC ATTAAGCGCG GCGGGTGTGG | 7740 |
| | TGGTTACGCG CAGCGTGACC GCTACACTTG CCAGCGCCCT AGCGCCCGCT CCTTTCGCTT | 7800 |
| | TCTTCCCTTC CTTTCTCGCC ACGTTCGCCG GCTTTCCCCG TCAAGCTCTA AATCGGGGGC | 7860 |
| | TCCCTTTAGG GTTCCGATTT AGTGCTTTAC GGCACCTCGA CCCCAAAAAA CTTGATTAGG | 7920 |
| | GTGATGGTTC ACGTAGTGGG CCATCGCCCT GATAGACGGT TTTTCGCCCT TTGACGTTGG | 7980 |
| | AGTCCACGTT CTTTAATAGT GGACTCTTGT TCCAAACTGG AACAACACTC AACCCTATCT | 8040 |
| | CGGTCTATTC TTTTGATTTA TAAGGGATTT TGCCGATTTC GGCCTATTGG TTAAAAAATG | 8100 |
| | AGCTGATTTA ACAAAAATTT AACGCGAATT AATTCTGTGG AATGTGTGTC AGTTAGGGTG | 8160 |
| | TGGAAAGTCC CCAGGCTCCC CAGCAGGCAG AAGTATGCAA AGCATGCATC TCAATTAGTC | 8220 |
| | AGCAACCAGG TGTGGAAAGT CCCCAGGCTC CCCAGCAGGC AGAAGTATGC AAAGCATGCA | 8280 |
| | TCTCAATTAG TCAGCAACCA TAGTCCCGCC CCTAACTCCG CCCATCCCGC CCCTAACTCC | 8340 |
| | GCCCAGTTCC GCCCATTCTC CGCCCCATGG CTGACTAATT TTTTTTATTT ATGCAGAGGC | 8400 |
| | CGAGGCCGCC TCTGCCTCTG AGCTATTCCA GAAGTAGTGA GGAGGCTTTT TTGGAGGCCT | 8460 |
| | AGGCTTTTGC AAAAAGCTCC CGGGAGCTTG TATATCCATT TTCGGATCTG ATCAGCACGT | 8520 |
| | GTTGACAATT AATCATCGGC ATAGTATATC GGCATAGTAT AATACGACAA GGTGAGGAAC | 8580 |
| | TAAACCATGG CCAAGTTGAC CAGTGCCGTT CCGGTGCTCA CCGCGCGCGA CGTCGCCGGA | 8640 |
| | GCGGTCGAGT TCTGGACCGA CCGGCTCGGG TTCTCCCGGG ACTTCGTGGA GGACGACTTC | 8700 |
| | GCCGGTGTGG TCCGGGACGA CGTGACCCTG TTCATCAGCG CGGTCCAGGA CCAGGTGGTG | 8760 |
| | CCGGACAACA CCCTGGCCTG GGTGTGGGTG CGCGGCCTGG ACGAGCTGTA CGCCGAGTGG | 8820 |
| | TCGGAGGTCG TGTCCACGAA CTTCCGGGAC GCCTCCGGGC CGGCCATGAC CGAGATCGGC | 8880 |
| | GAGCAGCCGT GGGGGCGGGA GTTCGCCCTG CGCGACCCGG CCGGCAACTG CGTGCACTTC | 8940 |
| | GTGGCCGAGG AGCAGGACTG ACACGTGCTA CGAGATTTCG ATTCCACCGC CGCCTTCTAT | 9000 |
| | GAAAGGTTGG GCTTCGGAAT CGTTTTCCGG GACGCCGGCT GGATGATCCT CCAGCGCGGG | 9060 |
| | GATCTCATGC TGGAGTTCTT CGCCCACCCC AACTTGTTTA TTGCAGCTTA TAATGGTTAC | 9120 |
| | AAATAAAGCA ATAGCATCAC AAATTTCACA AATAAAGCAT TTTTTTCACT GCATTCTAGT | 9180 |
| | TGTGGTTTGT CCAAACTCAT CAATGTATCT TATCATGTCT GTATACCGTC GACCTCTAGC | 9240 |
| | TAGAGCTTGG CGTAATCATG GTCATAGCTG TTTCCTGTGT GAAATTGTTA TCCGCTCACA | 9300 |
| | ATTCCACACA ACATACGAGC CGGAAGCATA AAGTGTAAAG CCTGGGGTGC CTAATGAGTG | 9360 |
| | AGCTAACTCA CATTAATTGC GTTGCGCTCA CTGCCCGCTT TCCAGTCGGG AAACCTGTCG | 9420 |
| | TGCCAGCTGC ATTAATGAAT CGGCCAACGC GCGGGGAGAG GCGGTTTGCG TATTGGGCGC | 9480 |
| | TCTTCCGCTT CCTCGCTCAC TGACTCGCTG CGCTCGGTCG TTCGGCTGCG GCGAGCGGTA | 9540 |
| | TCAGCTCACT CAAAGGCGGT AATACGGTTA TCCACAGAAT CAGGGGATAA CGCAGGAAAG | 9600 |
| | AACATGTGAG CAAAAGGCCA GCAAAAGGCC AGGAACCGTA AAAAGGCCGC GTTGCTGGCG | 9660 |
| | TTTTTCCATA GGCTCCGCCC CCCTGACGAG CATCACAAAA ATCGACGCTC AAGTCAGAGG | 9720 |
| | TGGCGAAACC CGACAGGACT ATAAAGATAC CAGGCGTTTC CCCCTGGAAG CTCCCTCGTG | 9780 |
| | CGCTCTCCTG TTCCGACCCT GCCGCTTACC GGATACCTGT CCGCCTTTCT CCCTTCGGGA | 9840 |
| | AGCGTGGCGC TTTCTCATAG CTCACGCTGT AGGTATCTCA GTTCGGTGTA GGTCGTTCGC | 9900 |
| | TCCAAGCTGG GCTGTGTGCA CGAACCCCCC GTTCAGCCCG ACCGCTGCGC CTTATCCGGT | 9960 |
| | AACTATCGTC TTGAGTCCAA CCCGGTAAGA CACGACTTAT CGCCACTGGC AGCAGCCACT | 10020 |
| | GGTAACAGGA TTAGCAGAGC GAGGTATGTA GGCGGTGCTA CAGAGTTCTT GAAGTGGTGG | 10080 |
| | CCTAACTACG GCTACACTAG AAGAACAGTA TTTGGTATCT GCGCTCTGCT GAAGCCAGTT | 10140 |
| | ACCTTCGGAA AAAGAGTTGG TAGCTCTTGA TCCGGCAAAC AAACCACCGC TGGTAGCGGT | 10200 |
| | GGTTTTTTTG TTTGCAAGCA GCAGATTACG CGCAGAAAAA AAGGATCTCA AGAAGATCCT | 10260 |
| | TTGATCTTTT CTACGGGGTC TGACGCTCAG TGGAACGAAA ACTCACGTTA AGGGATTTTG | 10320 |
| | GTCATGAGAT TATCAAAAAG GATCTTCACC TAGATCCTTT TAAATTAAAA ATGAAGTTTT | 10380 |
| | AAATCAATCT AAAGTATATA TGAGTAAACT TGGTCTGACA GTTACCAATG CTTAATCAGT | 10440 |
| | GAGGCACCTA TCTCAGCGAT CTGTCTATTT CGTTCATCCA TAGTTGCCTG ACTCCCCGTC | 10500 |
| | GTGTAGATAA CTACGATACG GGAGGGCTTA CCATCTGGCC CCAGTGCTGC AATGATACCG | 10560 |
| | CGAGACCCAC GCTCACCGGC TCCAGATTTA TCAGCAATAA ACCAGCCAGC CGGAAGGGCC | 10620 |
| | GAGCGCAGAA GTGGTCCTGC AACTTTATCC GCCTCCATCC AGTCTATTAA TTGTTGCCGG | 10680 |
| | GAAGCTAGAG TAAGTAGTTC GCCAGTTAAT AGTTTGCGCA ACGTTGTTGC CATTGCTACA | 10740 |
| | GGCATCGTGG TGTCACGCTC GTCGTTTGGT ATGGCTTCAT TCAGCTCCGG TTCCCAACGA | 10800 |
| | TCAAGGCGAG TTACATGATC CCCCATGTTG TGCAAAAAAG CGGTTAGCTC CTTCGGTCCT | 10860 |
| | CCGATCGTTG TCAGAAGTAA GTTGGCCGCA GTGTTATCAC TCATGGTTAT GGCAGCACTG | 10920 |
| | CATAATTCTC TTACTGTCAT GCCATCCGTA AGATGCTTTT CTGTGACTGG TGAGTACTCA | 10980 |
| | ACCAAGTCAT TCTGAGAATA GTGTATGCGG CGACCGAGTT GCTCTTGCCC GGCGTCAATA | 11040 |
| | CGGGATAATA CCGCGCCACA TAGCAGAACT TTAAAAGTGC TCATCATTGG AAAACGTTCT | 11100 |
| | TCGGGGCGAA AACTCTCAAG GATCTTACCG CTGTTGAGAT CCAGTTCGAT GTAACCCACT | 11160 |
| | CGTGCACCCA ACTGATCTTC AGCATCTTTT ACTTTCACCA GCGTTTCTGG GTGAGCAAAA | 11220 |
| | ACAGGAAGGC AAAATGCCGC AAAAAAGGGA ATAAGGGCGA CACGGAAATG TTGAATACTC | 11280 |
| | ATACTCTTCC TTTTTCAATA TTATTGAAGC ATTTATCAGG GTTATTGTCT CATGAGCGGA | 11340 |
| | TACATATTTG AATGTATTTA GAAAAATAAA CAAATAGGGG TTCCGCGCAC ATTTCCCCGA | 11400 |
| | AAAGTGCCAC CTGAC | 11415 |
| SEQ ID NO: 624 | GTCGACGGAT CGGGAGATCT CCCGATCCCC TATGGTGCAC TCTCAGTACA ATCTGCTCTG | 60 |
| pLentiCas9-EGFP | ATGCCGCATA GTTAAGCCAG TATCTGCTCC CTGCTTGTGT GTTGGAGGTC GCTGAGTAGT | 120 |
| vector | GCGCGAGCAA AATTTAAGCT ACAACAAGGC AAGGCTTGAC CGACAATTGC ATGAAGAATC | 180 |
| biepitope CCR | TGCTTAGGGT TAGGCGTTTT GCGCTGCTTC GCGATGTACG GGCCAGATAT ACGCGTTGAC | 240 |
| SP-(anti-TROP-2 | ATTGATTATT GACTAGTTAT TAATAGTAAT CAATTACGGG GTCATTAGTT CATAGCCCAT | 300 |
| SCEV)-(CD8 | ATATGGAGTT CCGCGTTACA TAACTTACGG TAAATGGCCC GCCTGGCTGA CCGCCCAACG | 360 |
| hinge)-(IL- | ACCCCCGCCC ATTGACGTCA ATAATGACGT ATGTTCCCAT AGTAACGCCA ATAGGGACTT | 420 |
| 18R1- | TCCATTGACG TCAATGGGTG GAGTATTTAC GGTAAACTGC CCACTTGGCA GTACATCAAG | 480 |
| transmembrane | TGTATCATAT GCCAAGTACG CCCCCTATTG ACGTCAATGA CGGTAAATGG CCCGCCTGGC | 540 |
| and | ATTATGCCCA GTACATGACC TTATGGGACT TTCCTACTTG GCAGTACATC TACGTATTAG | 600 |
| intracellular)- | TCATCGCTAT TACCATGGTG ATGCGGTTTT GGCAGTACAT CAATGGGCGT GGATAGCGGT | 660 |
| T2A-SP- | TTGACTCACG GGGASTTCCA AGTCTCCACC CCATTGACGT CAATGGGAGT TTGTTTTGGC | 720 |
| (anti-TROP-2 | ACCAAAATCA ACGGGACTTT CCAAAATGTC GTAACAACTC CGCCCCATTG ACGCAAATGG | 780 |
| SCEV)-(CD8 | GCGGTAGGCG TGTACGGTGG GAGGTCTATA TAAGCAGCGC GTTTTGCCTG TACTGGGTCT | 840 |
| hinge)-(IL- | CTCTGGTTAG ACCAGATCTG AGCCTGGGAG CTCTCTGGCT AACTAGGGAA CCCACTGCTT | 900 |
| 18RAP- | AAGCCTCAAT AAAGCTTGCC TTGAGTGCTT CAAGTAGTGT GTGCCCGTCT GTTGTGTGAC | 960 |
| transmembrane | TCTGGTAACT AGAGATCCCT CAGACCCTTT TAGTCAGTGT GGAAAATCTC TAGCAGTGGC | 1020 |
| and | GCCCGAACAG GGACTTGAAA GCGAAAGGGA AACCAGAGGA GCTCTCTCGA CGCAGGACTC | 1080 |
| intracellular) | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TGCCCGTCAC CGCCCTGTTA CTGCCTCTGG CCCTGTTGCT | 2940 |
| | GCACGCCGCC AGACCTGAGA TTGTGCTGAC GCAGAGCCCT GCCACCCTGT CTCTGTCTCC | 3000 |
| | TGGCGAGCGG GCCACACTGA GCTGCCGGGC CAGCCAGACC ATCGGCACAT CTATTCACTG | 3060 |
| | GTATCAACAG AAGCCCGGCC AGGCCCCTAG ACTGCTTATC TACTACGCCA GCGAAAGCAT | 3120 |
| | CAGCGGCATC CCAGCTCGGT TTAGCGGCAG CGGTTCCGGC ACCGACTTCA CACTGACGAT | 3180 |
| | CTCATCTTTG GAGCCTGAGG ACTTCGCCGT GTACTACTGC CAGCAAAGCA ACAGCTGGCC | 3240 |
| | TTTCACCTTC GGCCAGGGCA CCAAACTGGA AATCAAGGGC GGCGGGGGAT CGGGAGGCGG | 3300 |
| | CGGAAGCGGC GGAGGCGGCT CTGGCGGAGG AGGCTCTCAG GTTCAGCTGG TTCAATCTGG | 3360 |
| | AGCCGAAGTG AAGAAGCCTG GCGCCAGTGT GAAGGTGAGC TGCAAGGCCA GCGGCTACAC | 3420 |
| | CTTTACCAGC TACTGGATTA ACTGGGTCCG CCAGGCCCCT GGACAAGGCC TGGAGTGGAT | 3480 |
| | GGGCAACATC TACCCTAGCG ACAGCTACAG CAACTACAAC CAGAAGTTCA AGGACAGAGT | 3540 |
| | GACAATGACC AGGGACACCA GCACCAGCAC AGTCTATATG GAACTGAGCT CTCTGAGATC | 3600 |
| | CGAGGATACC GCCGTGTACT ACTGCGCTCG GGGTTCTAGC TTCGATTACT GGGGACAGGG | 3660 |
| | CACCCTGGTG ACAGETAGCA AGCCCACCAC AACCCCTGCC CCCCGGCCTC CGACCCCCGC | 3720 |
| | CCCCACAATC GCCAGCCAGC CTCTCTCGCT GAGACCTGAG GCTTGCAGAC CAGCCGCCGG | 3780 |
| | AGGAGCAGTG CACACCAGAG GCCTGGACTT TGCTTGTGAC ATCTACTACA GGGTCGACCT | 3840 |
| | GGTCCTGTTC TACCGGCACC TGACCAGAAG AGATGAGACA CTGACAGACG GCAAGACCTA | 3900 |
| | CGACGCCTTT GTGAGCTACC TGAAGGAATG CCGGCCTGAG AATGGCGAGG AACACACATT | 3960 |
| | CGCCGTGGAG ATCCTGCCTA GAGTGCTGGA GAAGCACTTC GGCTACAAGC TGTGCATCTT | 4020 |
| | CGAGAGAGAC GTGGTGCCGG GCGGAGCCGT GGTGGACGAA ATCCATTCTC TGATCGAGAA | 4080 |
| | GTCTCGGCGC CTGATTATCG TGCTGAGCAA GAGCTATATG AGCAACGAGG TGAGATACGA | 4140 |
| | GCTGGAAAGC GGCCTGCACG AGGCCCTGGT GGAAAGAAAG ATCAAAATCA TCCTGATCGA | 4200 |
| | GTTCACCCCT GTGACAGACT TCACCTTCCT GCCACAGAGC CTGAAGCTGC TGAAGAGCCA | 4260 |
| | CAGAGTGCTG AAATGGAAAG CAGATAAGTC CCTCAGCTAC AACTCACGGT TCTGGAAGAA | 4320 |
| | CCTGCTGTAC CTGATGCCCG CCAAAACCGT GAAGCCTGGC AGGGACGAGC CTGAGGTGCT | 4380 |
| | CCCAGTGCTA AGCGAAAGCG GCAGCGGAGA AGGCAGAGGC AGCCTGCTGA CCTGCGGGGA | 4440 |
| | TGTGGAAGAA AATCCTGGAC CTATGGCCCT GCCAGTGACC GCCCTGCTGC TGCCTCTGGC | 4500 |
| | CTTACTGCTG CATGCCGCTA GACCTGAGAT TGTGCTGACC CAGTCCCCCG CTACGTTGTC | 4560 |
| | TCTGAGCCCC GGCGAAAGAG CCACACTGAG CTGCCGGGCC AGTCAGACCA TCGGTACCAG | 4620 |
| | CATCCACTGG TACCAGCAGA AGCCTGGCCA GGCCCCAAGA CTGCTGATCT ATTACGCCTC | 4680 |
| | CGAGTCCATC AGCGGAATCC CTGCCAGATT CAGCGGCAGC GGATCTGGAA CAGATTTCAC | 4740 |
| | ACTGACCATC AGCAGCCTGG AACCCGAGGA CTTCGCCGTG TACTACTGCC AGCAGAGCAA | 4800 |
| | CAGCTGGCCC TTCACCTTCG GACAGGGCAC CAAGCTGGAA ATCAAGGGCG GCGGCGGGAG | 4860 |
| | CGGCGGGGGC GGCAGCGGCG GCGGCGGCAG CGGCGGTGGC GGCAGCCAGG TGCAGCTGGT | 4920 |
| | GCAGTCTGGT GCTGAGGTGA AAAAGCCTGG CGCCAGCGTG AAGGTGTCTT GTAAAGCCTC | 4980 |
| | GGGGTACACC TTCACCAGCT ATTGGATCAA CTGGGTGCGG CAGGCGCCCG GCCAGGGCCT | 5040 |
| | GGAGTGGATG GGCAACATCT ACCCCAGCGA CAGTTACAGC AATTACAATC AGAAATTCAA | 5100 |
| | GGACAGAGTG ACCATGACAC GGGATACATC CACCTCTACT GTGTACATGG AACTCTCCTC | 5160 |
| | TCTGCGGAGC GAAGACACCG CCGTGTACTA CTGTGCCAGA GGCAGTTCTT TCGACTACTG | 5220 |
| | GGGCCAGGGC ACTCTGGTGA CCGTCAGCAA ACCCACCACA ACTCCTGCAC CACGTCCCCC | 5280 |
| | GACACCTGCT CCCACCATTG CCTCTCAGCC CCTGAGCCTG CGGCCTGAGG CCTGCAGACC | 5340 |
| | CGCCGCTGGC GGCGCCGTTC ACACTAGAGG CCTCGACTTT GCCTGTGATA TCTACGGCGT | 5400 |
| | GTTACTGTAC ATCCTGCTGG GCACCATCGG AACCCTGGTG GCCGTGCTGG CCGCCAGCGC | 5460 |
| | CCTCCTGTAC AGACACTGGA TCGAGATCGT GCTGCTGTAT AGAACATACC AGTCCAAGGA | 5520 |
| | TCAGACGTTG GGCGACAAGA AGGATTTCGA CGCTTTTGTG TCTTACGCCA AGTGGTCGAG | 5580 |
| | CTTTCCCAGC GAGGCCACCT CATCTCTGAG CGAAGAGCAC CTGGCACTGT CTCTGTTCCC | 5640 |
| | TGACGTGCTG GAGAACAAGT ACGGCTACAG TCTGTGTCTG CTGGAACGGG ACGTGGCCCC | 5700 |
| | TGGCGGAGTG TATGCTGAAG ATATCGTGAG CATCATCAAA AGAAGCCGGC GCGGCATCTT | 5760 |
| | TATCCTGAGC CCCAACTACG TGAACGGCCC TAGCATCTTC GAGCTGCAGG CTGCCGTCAA | 5820 |
| | CCTGGCCCTG GATGACCAGA CCCTTAAGCT GATCCTGATC AAGTTCTGCT ACTTCCAGGA | 5880 |
| | GCCTGAATCC CTGCCCCACC TGGTGAAGAA GGCCCTGCGC GTGCTGCCTA CCGTGACCTG | 5940 |
| | GCGGGGCCTG AAGAGCGTGC CTCCTAACAG CAGATTCTGG GCCAAGATGA GATACCACAT | 6000 |
| | GCCTGTCAAA AACAGCCAAG GATTTACTTG GAACCAATTG AGAATCACTT CACGGATCTT | 6060 |
| | CCAATGGAAG GGCCTCTCTA GAACCGAGAC AACCGGCCGG AGCTCCCAAC CTAAGGAATG | 6120 |
| | GTGATAAGAA TTCGATATCA AGCTTATCGG TAATCAACCT CTGGATTACA AAATTTGTGA | 6180 |
| | AAGATTGACT GGTATTCTTA ACTATGTTGC TCCTTTTACG CTATGTGGAT ACGCTGCTTT | 6240 |
| | AATGCCTTTG TATCATGCTA TTGCTTCCCG TATGGCTTTC ATTTTCTCCT CCTTGTATAA | 6300 |
| | ATCCTGGTTG CTGTCTCTTT ATGAGGAGTT GTGGCCCGTT GTCAGGCAAC GTGGCGTGGT | 6360 |
| | GTGCACTGTG TTTGCTGACG CAACCCCCAC TGGTTGGGGC ATTGCCACCA CCTGTCAGCT | 6420 |
| | CCTTTCCGGG ACTTTCGCTT TCCCCCTCCC TATTGCCACG GCGGAACTCA TCGCCGCCTG | 6480 |
| | CCTTGCCCGC TGCTGGACAG GGGCTCGGCT GTTGGGCACT GACAATTCCG TGGTGTTGTC | 6540 |
| | GGGGAAATCA TCGTCCTTTC CTTGGCTGCT CGCCTGTGTT GCCACCTGGA TTCTGCGCGG | 6600 |
| | GACGTCCTTC TGCTACGTCC CTTCGGCCCT CAATCCAGCG GACCTTCCTT CCCGCGGCCT | 6660 |
| | GCTGCCGGCT CTGCGGCCTC TTCCGCGTCT TCGCCTTCGC CCTCAGACGA GTCGGATCTC | 6720 |
| | CCTTTGGGCC GCCTCCCCGC ATCGATACCG TCGACCTCGA GACCTAGAAA AACATGGAGC | 6780 |
| | AATCACAAGT AGCAATACAG CAGCTACCAA TGCTGATTGT GCCTGGCTAG AAGCACAAGA | 6840 |
| | GGAGGAGGAG GTGGGTTTTC CAGTCACACC TCAGGTACCT TTAAGACCAA TGACTTACAA | 6900 |
| | GGCAGCTGTA GATCTTAGCC ACTTTTTAAA AGAAAAGGGG GGACTGGAAG GGCTAATTCA | 6960 |
| | CTCCCAACGA AGACAAGATA TCCTTGATCT GTGGATCTAC CACACACAAG GCTACTTCCC | 7020 |
| | TGATTGGCAG AACTACACAC CAGGGCCAGG GATCAGATAT CCACTGACCT TTGGATGGTG | 7080 |
| | CTACAAGCTA GTACCAGTTG AGCAAGAGAA GGTAGAAGAA GCCAATGAAG GAGAGAACAC | 7140 |
| | CCGCTTGTTA CACCCTGTGA GCCTGCATGG GATGGATGAC CCGGAGAGAG AAGTATTAGA | 7200 |
| | GTGGAGGTTT GACAGCCGCC TAGCATTTCA TCACATGGCC CGAGAGCTGC ATCCGGACTG | 7260 |
| | TACTGGGTCT CTCTGGTTAG ACCAGATCTG AGCCTGGGAG CTCTCTGGCT AACTAGGGAA | 7320 |
| | CCCACTGCTT AAGCCTCAAT AAAGCTTGCC TTGAGTGCTT CAAGTAGTGT GTGCCCGTCT | 7380 |
| | GTTGTGTGAC TCTGGTAACT AGAGATCCCT CAGACCCTTT TAGTCAGTGT GGAAAATCTC | 7440 |
| | TAGCAGGGCC CGTTTAAACC CGCTGATCAG CCTCGACTGT GCCTTCTAGT TGCCAGCCAT | 7500 |
| | CTGTTGTTTG CCCCTCCCCC GTGCCTTCCT TGACCCTGGA AGGTGCCACT CCCACTGTCC | 7560 |
| | TTTCCTAATA AAATGAGGAA ATTGCATCGC ATTGTCTGAG TAGGTGTCAT TCTATTCTGG | 7620 |
| | GGGGTGGGGT GGGGCAGGAC AGCAAGGGGG AGGATTGGGA AGACAATAGC AGGCATGCTG | 7680 |
| | GGGATGCGGT GGGCTCTATG GCTTCTGAGG CGGAAAGAAC CAGCTGGGGC TCTAGGGGGT | 7740 |
| | ATCCCCACGC GCCCTGTAGC GGCGCATTAA GCGCGGCGGG TGTGGTGGTT ACGCGCAGCG | 7800 |
| | TGACCGCTAC ACTTGCCAGC GCCCTAGCGC CCGCTCCTTT CGCTTTCTTC CCTTCCTTTC | 7860 |
| | TCGCCACGTT CGCCGGCTTT CCCCGTCAAG CTCTAAATCG GGGGCTCCCT TTAGGGTTCC | 7920 |
| | GATTTAGTGC TTTACGGCAC CTCGACCCCA AAAAACTTGA TTAGGGTGAT GGTTCACGTA | 7980 |
| | GTGGGCCATC GCCCTGATAG ACGGTTTTTC GCCCTTTGAC GTTGGAGTCC ACGTTCTTTA | 8040 |
| | ATAGTGGACT CTTGTTCCAA ACTGGAACAA CACTCAACCC TATCTCGGTC TATTCTTTTG | 8100 |
| | ATTTATAAGG GATTTTGCCG ATTTCGGCCT ATTGGTTAAA AAATGAGCTG ATTTAACAAA | 8160 |
| | AATTTAACGC GAATTAATTC TGTGGAATGT GTGTCAGTTA GGGTGTGGAA AGTCCCCAGG | 8220 |
| | CTCCCCAGCA GGCAGAAGTA TGCAAAGCAT GCATCTCAAT TAGTCAGCAA CCAGGTGTGG | 8280 |
| | AAAGTCCCCA GGCTCCCCAG CAGGCAGAAG TATGCAAAGC ATGCATCTCA ATTAGTCAGC | 8340 |
| | AACCATAGTC CCGCCCCTAA CTCCGCCCAT CCCGCCCCTA ACTCCGCCCA GTTCCGCCCA | 8400 |
| | TTCTCCGCCC CATGGCTGAC TAATTTTTTT TATTTATGCA GAGGCCGAGG CCGCCTCTGC | 8460 |
| | CTCTGAGCTA TTCCAGAAGT AGTGAGGAGG CTTTTTTGGA GGCCTAGGCT TTTGCAAAAA | 8520 |
| | GCTCCCGGGA GCTTGTATAT CCATTTTCGG ATCTGATCAG CACGTGTTGA CAATTAATCA | 8580 |
| | TCGGCATAGT ATATCGGCAT AGTATAATAC GACAAGGTGA GGAACTAAAC CATGGCCAAG | 8640 |
| | TTGACCAGTG CCGTTCCGGT GCTCACCGCG CGCGACGTCG CCGGAGCGGT CGAGTTCTGG | 8700 |
| | ACCGACCGGC TCGGGTTCTC CCGGGACTTC GTGGAGGACG ACTTCGCCGG TGTGGTCCGG | 8760 |
| | GACGACGTGA CCCTGTTCAT CAGCGCGGTC CAGGACCAGG TGGTGCCGGA CAACACCCTG | 8820 |
| | GCCTGGGTGT GGGTGCGCGG CCTGGACGAG CTGTACGCCG AGTGGTCGGA GGTCGTGTCC | 8880 |
| | ACGAACTTCC GGGACGCCTC CGGGCCGGCC ATGACCGAGA TCGGCGAGCA GCCGTGGGGG | 8940 |
| | CGGGAGTTCG CCCTGCGCGA CCCGGCCGGC AACTGCGTGC ACTTCGTGGC CGAGGAGCAG | 9000 |
| | GACTGACACG TGCTACGAGA TTTCGATTCC ACCGCCGCCT TCTATGAAAG GTTGGGCTTC | 9060 |
| | GGAATCGTTT TCCGGGACGC CGGCTGGATG ATCCTCCAGC GCGGGGATCT CATGCTGGAG | 9120 |
| | TTCTTCGCCC ACCCCAACTT GTTTATTGCA GCTTATAATG GTTACAAATA AAGCAATAGC | 9180 |
| | ATCACAAATT TCACAAATAA AGCATTTTTT TCACTGCATT CTAGTTGTGG TTTGTCCAAA | 9240 |
| | CTCATCAATG TATCTTATCA TGTCTGTATA CCGTCGACCT CTAGCTAGAG CTTGGCGTAA | 9300 |
| | TCATGGTCAT AGCTGTTTCC TGTGTGAAAT TGTTATCCGC TCACAATTCC ACACAACATA | 9360 |
| | CGAGCCGGAA GCATAAAGTG TAAAGCCTGG GGTGCCTAAT GAGTGAGCTA ACTCACATTA | 9420 |
| | ATTGCGTTGC GCTCACTGCC CGCTTTCCAG TCGGGAAACC TGTCGTGCCA GCTGCATTAA | 9480 |
| | TGAATCGGCC AACGCGCGGG GAGAGGCGGT TTGCGTATTG GGCGCTCTTC CGCTTCCTCG | 9540 |
| | CTCACTGACT CGCTGCGCTC GGTCGTTCGG CTGCGGCGAG CGGTATCAGC TCACTCAAAG | 9600 |
| | GCGGTAATAC GGTTATCCAC AGAATCAGGG GATAACGCAG GAAAGAACAT GTGAGCAAAA | 9660 |
| | GGCCAGCAAA AGGCCAGGAA CCGTAAAAAG GCCGCGTTGC TGGCGTTTTT CCATAGGCTC | 9720 |
| | CGCCCCCCTG ACGAGCATCA CAAAAATCGA CGCTCAAGTC AGAGGTGGCG AAACCCGACA | 9780 |
| | GGACTATAAA GATACCAGGC GTTTCCCCCT GGAAGCTCCC TCGTGCGCTC TCCTGTTCCG | 9840 |
| | ACCCTGCCGC TTACCGGATA CCTGTCCGCC TTTCTCCCTT CGGGAAGCGT GGCGCTTTCT | 9900 |
| | CATAGCTCAC GCTGTAGGTA TCTCAGTTCG GTGTAGGTCG TTCGCTCCAA GCTGGGCTGT | 9960 |
| | GTGCACGAAC CCCCCGTTCA GCCCGACCGC TGCGCCTTAT CCGGTAACTA TCGTCTTGAG | 10020 |
| | TCCAACCCGG TAAGACACGA CTTATCGCCA CTGGCAGCAG CCACTGGTAA CAGGATTAGC | 10080 |
| | AGAGCGAGGT ATGTAGGCGG TGCTACAGAG TTCTTGAAGT GGTGGCCTAA CTACGGCTAC | 10140 |
| | ACTAGAAGAA CAGTATTTGG TATCTGCGCT CTGCTGAAGC CAGTTACCTT CGGAAAAAGA | 10200 |
| | GTTGGTAGCT CTTGATCCGG CAAACAAACC ACCGCTGGTA GCGGTGGTTT TTTTGTTTGC | 10260 |
| | AAGCAGCAGA TTACGCGCAG AAAAAAAGGA TCTCAAGAAG ATCCTTTGAT CTTTTCTACG | 10320 |
| | GGGTCTGACG CTCAGTGGAA CGAAAACTCA CGTTAAGGGA TTTTGGTCAT GAGATTATCA | 10380 |
| | AAAAGGATCT TCACCTAGAT CCTTTTAAAT TAAAAATGAA GTTTTAAATC AATCTAAAGT | 10440 |
| | ATATATGAGT AAACTTGGTC TGACAGTTAC CAATGCTTAA TCAGTGAGGC ACCTATCTCA | 10500 |
| | GCGATCTGTC TATTTCGTTC ATCCATAGTT GCCTGACTCC CCGTCGTGTA GATAACTACG | 10560 |
| | ATACGGGAGG GCTTACCATC TGGCCCCAGT GCTGCAATGA TACCGCGAGA CCCACGCTCA | 10620 |
| | CCGGCTCCAG ATTTATCAGC AATAAACCAG CCAGCCGGAA GGGCCGAGCG CAGAAGTGGT | 10680 |
| | CCTGCAACTT TATCCGCCTC CATCCAGTCT ATTAATTGTT GCCGGGAAGC TAGAGTAAGT | 10740 |
| | AGTTCGCCAG TTAATAGTTT GCGCAACGTT GTTGCCATTG CTACAGGCAT CGTGGTGTCA | 10800 |
| | CGCTCGTCGT TTGGTATGGC TTCATTCAGC TCCGGTTCCC AACGATCAAG GCGAGTTACA | 10860 |
| | TGATCCCCCA TGTTGTGCAA AAAAGCGGTT AGCTCCTTCG GTCCTCCGAT CGTTGTCAGA | 10920 |
| | AGTAAGTTGG CCGCAGTGTT ATCACTCATG GTTATGGCAG CACTGCATAA TTCTCTTACT | 10980 |
| | GTCATGCCAT CCGTAAGATG CTTTTCTGTG ACTGGTGAGT ACTCAACCAA GTCATTCTGA | 11040 |
| | GAATAGTGTA TGCGGCGACC GAGTTGCTCT TGCCCGGCGT CAATACGGGA TAATACCGCG | 11100 |
| | CCACATAGCA GAACTTTAAA AGTGCTCATC ATTGGAAAAC GTTCTTCGGG GCGAAAACTC | 11160 |
| | TCAAGGATCT TACCGCTGTT GAGATCCAGT TCGATGTAAC CCACTCGTGC ACCCAACTGA | 11220 |
| | TCTTCAGCAT CTTTTACTTT CACCAGCGTT TCTGGGTGAG CAAAAACAGG AAGGCAAAAT | 11280 |
| | GCCGCAAAAA AGGGAATAAG GGCGACACGG AAATGTTGAA TACTCATACT CTTCCTTTTT | 11340 |
| | CAATATTATT GAAGCATTTA TCAGGGTTAT TGTCTCATGA GCGGATACAT ATTTGAATGT | 11400 |
| | ATTTAGAAAA ATAAACAAAT AGGGGTTCCG CGCACATTTC CCCGAAAAGT GCCACCTGAC | 11460 |
| SEQ ID NO: 625 | GTCGACGGAT CGGGAGATCT CCCGATCCCC TATGGTGCAC TCTCAGTACA ATCTGCTCTG | 60 |
| pLentiCas9-EGFP | ATGCCGCATA GTTAAGCCAG TATCTGCTCC CTGCTTGTGT GTTGGAGGTC GCTGAGTAGT | 120 |
| vector | GCGCGAGCAA AATTTAAGCT ACAACAAGGC AAGGCTTGAC CGACAATTGC ATGAAGAATC | 180 |
| biepitope CCR | TGCTTAGGGT TAGGCGTTTT GCGCTGCTTC GCGATGTACG GGCCAGATAT ACGCGTTGAC | 240 |
| SP-(cAR47A6.4 | ATTGATTATT GACTAGTTAT TAATAGTAAT CAATTACGGG GTCATTAGTT CATAGCCCAT | 300 |
| SCEV)-(CD28 | ATATGGAGTT CCGCGTTACA TAACTTACGG TAAATGGCCC GCCTGGCTGA CCGCCCAACG | 360 |
| hinge- | ACCCCCGCCC ATTGACGTCA ATAATGACGT ATGTTCCCAT AGTAACGCCA ATAGGGACTT | 420 |
| transmembrane- | TCCATTGACG TCAATGGGTG GAGTATTTAC GGTAAACTGC CCACTTGGCA GTACATCAAG | 480 |
| (IL-2Rβ | TGTATCATAT GCCAAGTACG CCCCCTATTG ACGTCAATGA CGGTAAATGG CCCGCCTGGC | 540 |
| intracellular)- | ATTATGCCCA GTACATGACC TTATGGGACT TTCCTACTTG GCAGTACATC TACGTATTAG | 600 |
| T2A-SP-(KM4097 | TCATCGCTAT TACCATGGTG ATGCGGTTTT GGCAGTACAT CAATGGGCGT GGATAGCGGT | 660 |
| SCEv)-(CD28 | TTGACTCACG GGGASTTCCA AGTCTCCACC CCATTGACGT CAATGGGAGT TTGTTTTGGC | 720 |
| hinge and | ACCAAAATCA ACGGGACTTT CCAAAATGTC GTAACAACTC CGCCCCATTG ACGCAAATGG | 780 |
| transmembrane)- | GCGGTAGGCG TGTACGGTGG GAGGTCTATA TAAGCAGCGC GTTTTGCCTG TACTGGGTCT | 840 |
| (IL-2Rγ | CTCTGGTTAG ACCAGATCTG AGCCTGGGAG CTCTCTGGCT AACTAGGGAA CCCACTGCTT | 900 |
| intracellular) | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TGCCGGTCAC CGCCCTGCTG CTGCCCCTGG CCCTACTGCT | 2940 |
| | GCATGCCGCT AGGCCTCAGA TCCAGCTCGT GCAGAGCGGT CCTGAGCTGA AGAAACCCGG | 3000 |
| | CGAGACAGTG AAAATCAGCT GCAAGGCCAG CGGCTACACA TTCACCAACT ATGGCATGAA | 3060 |
| | CTGGGTGAAG CAGGCCCCTG GGAAGGGCCT GAAGTGGATG GGCTGGATCA ATACAAAGAC | 3120 |
| | CGGCGAACCC ACCTACGCCG AGGAGTTCAA GGGCAGATTC GCCTTTAGCT TGGAGACAAG | 3180 |
| | TGCCAGCACA GCCTACCTGC AAATTAACAA CCTGAAAAAA GAGGACACCG CCACCTACTT | 3240 |
| | TTGCGGCAGA GGCGGCTATG GCAGCAGCTA CTGGTACTTC GACGTGTGGG GCGCCGGCAC | 3300 |
| | CACCGTCACA GTGAGCAGCG CCTCTACAAA GGGCCCTAGT GGCGGAGGCG GGTCCGGTGG | 3360 |
| | GGGCGGGTCT GGAGGCGGCG GCAGCGGCGG CGGCGGCAGC GACATCGTGA TGACCCAAAG | 3420 |
| | CCACAAGTTC ATGAGCACAA GCGTGGGCGA CCGGGTGAGC ATCACCTGCA AGGCCAGCCA | 3480 |
| | GGATGTGTCT ATCGCCGTGG CCTGGTACCA GCAAAAGCCG GGCCAGAGCC CCAAAGTGCT | 3540 |
| | GATCTATTCC GCTTCTTATA GATACACCGG AGTTCCTGAT AGGTTCACCG GATCCGGCTC | 3600 |
| | CGGAACAGAC TTCACATTCA CCATCAGCAG AGTTCAAGCT GAGGACCTGG CCGTGTACTA | 3660 |
| | CTGCCAGCAG CACTACATTA CCCCTCTCAC CTTCGGCGCC GGGACCAAGC TGGAGCTGAA | 3720 |
| | AAGAACAGTA GCCATCGAGG TGATGTACCC ACCTCCTTAC CTGGACAATG AAAAGAGCAA | 3780 |
| | CGGCACCATC ATCCACGTCA AGGGCAAACA CCTGTGTCCT TCCCCACTGT TCCCTGGACC | 3840 |
| | CTCTAAGCCT TTTTGGGTGC TGGTCGTGGT GGGCGGCGTG CTGGCCTGCT ACAGCCTGCT | 3900 |
| | GGTGACCGTG GCCTTCATCA TCTTCTGGGT GAACTGCAGA AACACCGGCC CTTGGCTGAA | 3960 |
| | GAAGGTGCTG AAGTGTAACA CCCCTGATCC TTCCAAGTTT TTCAGCCAGC TGAGCAGCGA | 4020 |
| | GCACGGCGGA GACGETCAGA AGTGGCTGTC TAGCCCATTC CCTAGCAGCT CTTTCAGTCC | 4080 |
| | TGGCGGGCTG GCCCCTGAGA TCTCTCCTCT TGAAGTGCTG GAAAGAGATA AGGTGACACA | 4140 |
| | GCTGCTCCTG CAGCAGGACA AGGTGCCAGA ACCTGCCAGC CTGAGCAGCA ATCACTCTCT | 4200 |
| | AACATCTTGC TTCACCAATC AGGGCTACTT CTTCTTTCAC CTGCCTGACG CCCTGGAGAT | 4260 |
| | CGAGGCCTGT CAGGTGTACT TCACATACGA CCCCTACAGC GAGGAAGATC CTGACGAGGG | 4320 |
| | AGTTGCCGGA GCTCCAACAG GATCTTCTCC ACAGCCCCTG CAGCCTCTGA GCGGAGAAGA | 4380 |
| | TGACGCTTAT TGTACATTTC CCAGCAGAGA CGACCTACTG CTCTTCAGTC CTAGCCTCCT | 4440 |
| | GGGCGGCCCC TCTCCACCTA GCACCGCCCC TGGGGGCAGC GGCGCCGGCG AAGAACGGAT | 4500 |
| | GCCCCCTAGT CTCCAGGAGC GGGTGCCACG GGACTGGGAT CCTCAGCCTC TGGGCCCCCC | 4560 |
| | AACCCCTGGT GTGCCTGATC TGGTCGACTT CCAGCCTCCT CCGGAACTGG TGCTGAGGGA | 4620 |
| | AGCCGGCGAG GAAGTGCCAG ACGCCGGCCC CAGAGAGGGT GTGAGCTTTC CATGGTCCCG | 4680 |
| | GCCCCCCGGC CAGGGCGAGT TCAGAGCCCT GAATGCCCGG CTGCCTCTGA ACACAGATGC | 4740 |
| | TTACCTCAGC CTTCAAGAGC TGCAGGGCCA GGACCCTACC CACCTGGTCG GCTCAGGCGA | 4800 |
| | GGGCAGAGGC AGCCTGCTGA CATGCGGCGA CGTGGAAGAA AACCCCGGCC CCATGGCCCT | 4860 |
| | GCCTGTGACC GCCCTCCTGC TGCCTCTGGC ACTGCTGCTG CACGCCGCCA GACCTCAGGT | 4920 |
| | GCAGCTGCAG CAGTCTGGCC CCGAACTGGT GCGGCCCGGA ACCTCTGTGC GGATCAGCTG | 4980 |
| | CAAAGCCTCC GGCTACACCT TTACCATCTA CTGGCTGGGC TGGGTGAAGC AGAGACCAGG | 5040 |
| | TCACGGCCTG GAGTGGATAG GAAACATCTT CCCTGGCAGT GCCTACATCA ACTACAACGA | 5100 |
| | GAAGTTCAAG GGCAAGGCTA CCCTGACCGC TGACACCTCT TCCAGCACCG CCTACATGCA | 5160 |
| | GCTGAGCAGC CTGACAAGCG AGGACAGCGC CGTGTACTTC TGCGCCAGAG AGGGAAGCAA | 5220 |
| | CAGCGGCTAC TGGGGCCAAG GCACGACCCT GACCGTGAGC TCTGGCGGCG GTGGATCTGG | 5280 |
| | CGGCGGCGGT TCCGGAGGCG GAGGGTCTGG AGGTGGCGGC AGCGACATCG TGATGACTCA | 5340 |
| | GAGCCCGTCT AGCCTGAGCG TGTCTGCCGG AGAGAAGGTG ACCATGACAT GCAAGTCCAG | 5400 |
| | CCAAAGCCTG CTGAACAGCG GCAACCAGCA GAACTACCTG GCTTGGTATC AGCAGAAACC | 5460 |
| | CGGCCAGCCG CCAAAGCTGC TCATCTACGG CGCCAGCACA AGAGAGAGCG GCGTGCCCGA | 5520 |
| | TAGATTCACC GGCTCGGGCT CTGGAACCGA CTTCACTCTG ACCATCAACA GCGTGCAAGC | 5580 |
| | TGAGGATCTG GCCGTGTACT ACTGTCAGTC TGACCACATC TACCCCTATA CATTCGGGGG | 5640 |
| | AGGCACAAAA CTGGAAATCA AGATCGAAGT GATGTACCCT CCACCTTACC TGGATAACGA | 5700 |
| | GAAGAGCAAC GGGACCATCA TCCACGTGAA GGGAAAGCAT CTGTGCCCCT CGCCCCTGTT | 5760 |
| | CCCCGGCCCG AGCAAGCCTT TTTGGGTGCT CGTGGTGGTG GGCGGCGTGC TGGCCTGCTA | 5820 |
| | CTCTCTGCTG GTGACCGTGG CCTTCATCAT CTTCTGGGTT GAGCGGACCA TGCCTAGAAT | 5880 |
| | CCCCACCCTG AAAAACCTGG AGGATCTGGT GACCGAGTAC CACGGCAACT TCTCGGCTTG | 5940 |
| | GTCCGGCGTG AGCAAGGGCC TGGCCGAAAG CCTGCAGCCT GACTACAGCG AACGGCTGTG | 6000 |
| | CCTGGTCAGC GAGATTCCTC CTAAAGGCGG CGCCCTGGGA GAGGGCCCTG GCGCCTCACC | 6060 |
| | TTGTAACCAG CACAGCCCTT ACTGGGCGCC TCCTTGCTAC ACCCTGAAGC CTGAGACATG | 6120 |
| | ATAAGAATTC GATASCAAGC TTATCGGTAA TCAACCTCTG GATTACAAAA TTTGTGAAAG | 6180 |
| | ATTGACTGGT ATTCTTAACT ATGTTGCTCC TTTTACGCTA TGTGGATACG CTGCTTTAAT | 6240 |
| | GCCTTTGTAT CATGCTATTG CTTCCCGTAT GGCTTTCATT TTCTCCTCCT TGTATAAATC | 6300 |
| | CTGGTTGCTG TCTCETTATG AGGAGTTGTG GCCCGTTGTC AGGCAACGTG GCGTGGTGTG | 6360 |
| | CACTGTGTTT GCTGACGCAA CCCCCACTGG TTGGGGCATT GCCACCACCT GTCAGCTCCT | 6420 |
| | TTCCGGGACT TTCGCTTTCC CCCTCCCTAT TGCCACGGCG GAACTCATCG CCGCCTGCCT | 6480 |
| | TGCCCGCTGC TGGACAGGGG CTCGGCTGTT GGGCACTGAC AATTCCGTGG TGTTGTCGGG | 6540 |
| | GAAATCATCG TCCTTTCCTT GGCTGCTCGC CTGTGTTGCC ACCTGGATTC TGCGCGGGAC | 6600 |
| | GTCCTTCTGC TACGTCCCTT CGGCCCTCAA TCCAGCGGAC CTTCCTTCCC GCGGCCTGCT | 6660 |
| | GCCGGCTCTG CGGCCTCTTC CGCGTCTTCG CCTTCGCCCT CAGACGAGTC GGATCTCCCT | 6720 |
| | TTGGGCCGCC TCCCCGCATC GATACCGTCG ACCTCGAGAC CTAGAAAAAC ATGGAGCAAT | 6780 |
| | CACAAGTAGC AATACAGCAG CTACCAATGC TGATTGTGCC TGGCTAGAAG CACAAGAGGA | 6840 |
| | GGAGGAGGTG GGTTTTCCAG TCACACCTCA GGTACCTTTA AGACCAATGA CTTACAAGGC | 6900 |
| | AGCTGTAGAT CTTAGCCACT TTTTAAAAGA AAAGGGGGGA CTGGAAGGGC TAATTCACTC | 6960 |
| | CCAACGAAGA CAAGATATCC TTGATCTGTG GATCTACCAC ACACAAGGCT ACTTCCCTGA | 7020 |
| | TTGGCAGAAC TACACACCAG GGCCAGGGAT CAGATATCCA CTGACCTTTG GATGGTGCTA | 7080 |
| | CAAGCTAGTA CCAGTTGAGC AAGAGAAGGT AGAAGAAGCC AATGAAGGAG AGAACACCCG | 7140 |
| | CTTGTTACAC CCTGTGAGCC TGCATGGGAT GGATGACCCG GAGAGAGAAG TATTAGAGTG | 7200 |
| | GAGGTTTGAC AGCCGCCTAG CATTTCATCA CATGGCCCGA GAGCTGCATC CGGACTGTAC | 7260 |
| | TGGGTCTCTC TGGTTAGACC AGATCTGAGC CTGGGAGCTC TCTGGCTAAC TAGGGAACCC | 7320 |
| | ACTGCTTAAG CCTCAATAAA GCTTGCCTTG AGTGCTTCAA GTAGTGTGTG CCCGTCTGTT | 7380 |
| | GTGTGACTCT GGTAACTAGA GATCCCTCAG ACCCTTTTAG TCAGTGTGGA AAATCTCTAG | 7440 |
| | CAGGGCCCGT TTAAACCCGC TGATCAGCCT CGACTGTGCC TTCTAGTTGC CAGCCATCTG | 7500 |
| | TTGTTTGCCC CTCCCCCGTG CCTTCCTTGA CCCTGGAAGG TGCCACTCCC ACTGTCCTTT | 7560 |
| | CCTAATAAAA TGAGGAAATT GCATCGCATT GTCTGAGTAG GTGTCATTCT ATTCTGGGGG | 7620 |
| | GTGGGGTGGG GCAGGACAGC AAGGGGGAGG ATTGGGAAGA CAATAGCAGG CATGCTGGGG | 7680 |
| | ATGCGGTGGG CTCTATGGCT TCTGAGGCGG AAAGAACCAG CTGGGGCTCT AGGGGGTATC | 7740 |
| | CCCACGCGCC CTGTAGCGGC GCATTAAGCG CGGCGGGTGT GGTGGTTACG CGCAGCGTGA | 7800 |
| | CCGCTACACT TGCCAGCGCC CTAGCGCCCG CTCCTTTCGC TTTCTTCCCT TCCTTTCTCG | 7860 |
| | CCACGTTCGC CGGCETTCCC CGTCAAGCTC TAAATCGGGG GCTCCCTTTA GGGTTCCGAT | 7920 |
| | TTAGTGCTTT ACGGCACCTC GACCCCAAAA AACTTGATTA GGGTGATGGT TCACGTAGTG | 7980 |
| | GGCCATCGCC CTGATAGACG GTTTTTCGCC CTTTGACGTT GGAGTCCACG TTCTTTAATA | 8040 |
| | GTGGACTCTT GTTCCAAACT GGAACAACAC TCAACCCTAT CTCGGTCTAT TCTTTTGATT | 8100 |
| | TATAAGGGAT TTTGCCGATT TCGGCCTATT GGTTAAAAAA TGAGCTGATT TAACAAAAAT | 8160 |
| | TTAACGCGAA TTAATTCTGT GGAATGTGTG TCAGTTAGGG TGTGGAAAGT CCCCAGGCTC | 8220 |
| | CCCAGCAGGC AGAAGTATGC AAAGCATGCA TCTCAATTAG TCAGCAACCA GGTGTGGAAA | 8280 |
| | GTCCCCAGGC TCCCCAGCAG GCAGAAGTAT GCAAAGCATG CATCTCAATT AGTCAGCAAC | 8340 |
| | CATAGTCCCG CCCCTAACTC CGCCCATCCC GCCCCTAACT CCGCCCAGTT CCGCCCATTC | 8400 |
| | TCCGCCCCAT GGCTGACTAA TTTTTTTTAT TTATGCAGAG GCCGAGGCCG CCTCTGCCTC | 8460 |
| | TGAGCTATTC CAGAAGTAGT GAGGAGGCTT TTTTGGAGGC CTAGGCTTTT GCAAAAAGCT | 8520 |
| | CCCGGGAGCT TGTATATCCA TTTTCGGATC TGATCAGCAC GTGTTGACAA TTAATCATCG | 8580 |
| | GCATAGTATA TCGGCATAGT ATAATACGAC AAGGTGAGGA ACTAAACCAT GGCCAAGTTG | 8640 |
| | ACCAGTGCCG TTCCGGTGCT CACCGCGCGC GACGTCGCCG GAGCGGTCGA GTTCTGGACC | 8700 |
| | GACCGGCTCG GGTTCTCCCG GGACTTCGTG GAGGACGACT TCGCCGGTGT GGTCCGGGAC | 8760 |
| | GACGTGACCC TGTTCATCAG CGCGGTCCAG GACCAGGTGG TGCCGGACAA CACCCTGGCC | 8820 |
| | TGGGTGTGGG TGCGCGGCCT GGACGAGCTG TACGCCGAGT GGTCGGAGGT CGTGTCCACG | 8880 |
| | AACTTCCGGG ACGCCTCCGG GCCGGCCATG ACCGAGATCG GCGAGCAGCC GTGGGGGCGG | 8940 |
| | GAGTTCGCCC TGCGCGACCC GGCCGGCAAC TGCGTGCACT TCGTGGCCGA GGAGCAGGAC | 9000 |
| | TGACACGTGC TACGAGATTT CGATTCCACC GCCGCCTTCT ATGAAAGGTT GGGCTTCGGA | 9060 |
| | ATCGTTTTCC GGGACGCCGG CTGGATGATC CTCCAGCGCG GGGATCTCAT GCTGGAGTTC | 9120 |
| | TTCGCCCACC CCAACTTGTT TATTGCAGCT TATAATGGTT ACAAATAAAG CAATAGCATC | 9180 |
| | ACAAATTTCA CAAATAAAGC ATTTTTTTCA CTGCATTCTA GTTGTGGTTT GTCCAAACTC | 9240 |
| | ATCAATGTAT CTTATCATGT CTGTATACCG TCGACCTCTA GCTAGAGCTT GGCGTAATCA | 9300 |
| | TGGTCATAGC TGTTTCCTGT GTGAAATTGT TATCCGCTCA CAATTCCACA CAACATACGA | 9360 |
| | GCCGGAAGCA TAAAGTGTAA AGCCTGGGGT GCCTAATGAG TGAGCTAACT CACATTAATT | 9420 |
| | GCGTTGCGCT CACTGCCCGC TTTCCAGTCG GGAAACCTGT CGTGCCAGCT GCATTAATGA | 9480 |
| | ATCGGCCAAC GCGCGGGGAG AGGCGGTTTG CGTATTGGGC GCTCTTCCGC TTCCTCGCTC | 9540 |
| | ACTGACTCGC TGCGCTCGGT CGTTCGGCTG CGGCGAGCGG TATCAGCTCA CTCAAAGGCG | 9600 |
| | GTAATACGGT TATCCACAGA ATCAGGGGAT AACGCAGGAA AGAACATGTG AGCAAAAGGC | 9660 |
| | CAGCAAAAGG CCAGGAACCG TAAAAAGGCC GCGTTGCTGG CGTTTTTCCA TAGGCTCCGC | 9720 |
| | CCCCCTGACG AGCATCACAA AAATCGACGC TCAAGTCAGA GGTGGCGAAA CCCGACAGGA | 9780 |
| | CTATAAAGAT ACCAGGCGTT TCCCCCTGGA AGCTCCCTCG TGCGCTCTCC TGTTCCGACC | 9840 |
| | CTGCCGCTTA CCGGATACCT GTCCGCCTTT CTCCCTTCGG GAAGCGTGGC GCTTTCTCAT | 9900 |
| | AGCTCACGCT GTAGGTATCT CAGTTCGGTG TAGGTCGTTC GCTCCAAGCT GGGCTGTGTG | 9960 |
| | CACGAACCCC CCGTTCAGCC CGACCGCTGC GCCTTATCCG GTAACTATCG TCTTGAGTCC | 10020 |
| | AACCCGGTAA GACACGACTT ATCGCCACTG GCAGCAGCCA CTGGTAACAG GATTAGCAGA | 10080 |
| | GCGAGGTATG TAGGCGGTGC TACAGAGTTC TTGAAGTGGT GGCCTAACTA CGGCTACACT | 10140 |
| | AGAAGAACAG TATTTGGTAT CTGCGCTCTG CTGAAGCCAG TTACCTTCGG AAAAAGAGTT | 10200 |
| | GGTAGCTCTT GATCCGGCAA ACAAACCACC GCTGGTAGCG GTGGTTTTTT TGTTTGCAAG | 10260 |
| | CAGCAGATTA CGCGCAGAAA AAAAGGATCT CAAGAAGATC CTTTGATCTT TTCTACGGGG | 10320 |
| | TCTGACGCTC AGTGGAACGA AAACTCACGT TAAGGGATTT TGGTCATGAG ATTATCAAAA | 10380 |
| | AGGATCTTCA CCTAGATCCT TTTAAATTAA AAATGAAGTT TTAAATCAAT CTAAAGTATA | 10440 |
| | TATGAGTAAA CTTGGTCTGA CAGTTACCAA TGCTTAATCA GTGAGGCACC TATCTCAGCG | 10500 |
| | ATCTGTCTAT TTCGTTCATC CATAGTTGCC TGACTCCCCG TCGTGTAGAT AACTACGATA | 10560 |
| | CGGGAGGGCT TACCATCTGG CCCCAGTGCT GCAATGATAC CGCGAGACCC ACGCTCACCG | 10620 |
| | GCTCCAGATT TATCAGCAAT AAACCAGCCA GCCGGAAGGG CCGAGCGCAG AAGTGGTCCT | 10680 |
| | GCAACTTTAT CCGCCTCCAT CCAGTCTATT AATTGTTGCC GGGAAGCTAG AGTAAGTAGT | 10740 |
| | TCGCCAGTTA ATAG-TTGCG CAACGTTGTT GCCATTGCTA CAGGCATCGT GGTGTCACGC | 10800 |
| | TCGTCGTTTG GTATGGCTTC ATTCAGCTCC GGTTCCCAAC GATCAAGGCG AGTTACATGA | 10860 |
| | TCCCCCATGT TGTGCAAAAA AGCGGTTAGC TCCTTCGGTC CTCCGATCGT TGTCAGAAGT | 10920 |
| | AAGTTGGCCG CAGTGTTATC ACTCATGGTT ATGGCAGCAC TGCATAATTC TCTTACTGTC | 10980 |
| | ATGCCATCCG TAAGATGCTT TTCTGTGACT GGTGAGTACT CAACCAAGTC ATTCTGAGAA | 11040 |
| | TAGTGTATGC GGCGACCGAG TTGCTCTTGC CCGGCGTCAA TACGGGATAA TACCGCGCCA | 11100 |
| | CATAGCAGAA CTTTAAAAGT GCTCATCATT GGAAAACGTT CTTCGGGGCG AAAACTCTCA | 11160 |
| | AGGATCTTAC CGCTGTTGAG ATCCAGTTCG ATGTAACCCA CTCGTGCACC CAACTGATCT | 11220 |
| | TCAGCATCTT TTACTTTCAC CAGCGTTTCT GGGTGAGCAA AAACAGGAAG GCAAAATGCC | 11280 |
| | GCAAAAAAGG GAATAAGGGC GACACGGAAA TGTTGAATAC TCATACTCTT CCTTTTTCAA | 11340 |
| | TATTATTGAA GCATTTATCA GGGTTATTGT CTCATGAGCG GATACATATT TGAATGTATT | 11400 |
| | TAGAAAAATA AACAAATAGG GGTTCCGCGC ACATTTCCCC GAAAAGTGCC ACCTGAC | 11457 |
| SEQ ID NO: 626 | GTCGACGGAT CGGGAGATCT CCCGATCCCC TATGGTGCAC TCTCAGTACA ATCTGCTCTG | 60 |
| pLentiCas9-EGFP | ATGCCGCATA GTTAAGCCAG TATCTGCTCC CTGCTTGTGT GTTGGAGGTC GCTGAGTAGT | 120 |
| vector | GCGCGAGCAA AATTTAAGCT ACAACAAGGC AAGGCTTGAC CGACAATTGC ATGAAGAATC | 180 |
| biepitope CCR | TGCTTAGGGT TAGGCGTTTT GCGCTGCTTC GCGATGTACG GGCCAGATAT ACGCGTTGAC | 240 |
| SP-(cAR47A6.4 | ATTGATTATT GACTAGTTAT TAATAGTAAT CAATTACGGG GTCATTAGTT CATAGCCCAT | 300 |
| SCEV)-(CD28 | ATATGGAGTT CCGCGTTACA TAACTTACGG TAAATGGCCC GCCTGGCTGA CCGCCCAACG | 360 |
| hinge- | ACCCCCGCCC ATTGACGTCA ATAATGACGT ATGTTCCCAT AGTAACGCCA ATAGGGACTT | 420 |
| transmembrane)- | TCCATTGACG TCAATGGGTG GAGTATTTAC GGTAAACTGC CCACTTGGCA GTACATCAAG | 480 |
| (IL-18R1 | TGTATCATAT GCCAAGTACG CCCCCTATTG ACGTCAATGA CGGTAAATGG CCCGCCTGGC | 540 |
| intracellular)- | ATTATGCCCA GTACATGACC TTATGGGACT TTCCTACTTG GCAGTACATC TACGTATTAG | 600 |
| T2A-SP- | TCATCGCTAT TACCATGGTG ATGCGGTTTT GGCAGTACAT CAATGGGCGT GGATAGCGGT | 660 |
| ( KM4097scFv)- | TTGACTCACG GGGATTTCCA AGTCTCCACC CCATTGACGT CAATGGGAGT TTGTTTTGGC | 720 |
| (CD28 hinge- | ACCAAAATCA ACGGGACTTT CCAAAATGTC GTAACAACTC CGCCCCATTG ACGCAAATGG | 780 |
| transmembrane)- | GCGGTAGGCG TGTACGGTGG GAGGTCTATA TAAGCAGCGC GTTTTGCCTG TACTGGGTCT | 840 |
| (IL-18RAP | CTCTGGTTAG ACCAGATCTG AGCCTGGGAG CTCTCTGGCT AACTAGGGAA CCCACTGCTT | 900 |
| intracellular) | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TGCCGGTGAC AGCCCTGTTA CTGCCTCTGG CCCTGCTGCT | 2940 |
| | TCATGCCGCC AGGCCTCAAA TCCAGCTGGT GCAGTCCGGC CCCGAACTGA AGAAGCCCGG | 3000 |
| | AGAGACCGTG AAGATCAGCT GCAAGGCCTC AGGGTACACA TTTACAAACT ACGGCATGAA | 3060 |
| | CTGGGTGAAA CAGGCCCCTG GCAAGGGACT GAAGTGGATG GGCTGGATCA ACACCAAGAC | 3120 |
| | AGGCGAGCCT ACATACGCCG AAGAGTTCAA GGGACGGTTT GCCTTTAGCT TGGAGACCTC | 3180 |
| | TGCTAGTACC GCTTACCTGC AGATCAACAA CCTGAAAAAA GAGGACACCG CCACCTACTT | 3240 |
| | CTGCGGCAGA GGCGGGTACG GCAGCAGCTA CTGGTACTTC GACGTGTGGG GAGCCGGCAC | 3300 |
| | TACCGTGACT GTGAGCAGCG CCAGCACCAA GGGCCCTAGC GGCGGCGGCG GCAGCGGAGG | 3360 |
| | CGGCGGATCT GGGGGCGGCG GCTCAGGCGG CGGAGGTAGC GACATCGTGA TGACCCAGAG | 3420 |
| | CCATAAGTTC ATGAGCACAA GCGTGGGCGA CAGAGTGAGC ATCACATGTA AGGCCTCCCA | 3480 |
| | GGACGTCTCT ATCGCCGTGG CCTGGTACCA GCAGAAGCCT GGCCAGAGCC CTAAGGTGCT | 3540 |
| | GATCTATAGC GCCAGCTACA GATACACGGG AGTGCCAGAT AGATTCACAG GCAGCGGGTC | 3600 |
| | TGGTACAGAC TTCACCTTCA CCATAAGCAG AGTGCAGGCC GAGGACCTGG CCGTCTACTA | 3660 |
| | CTGCCAGCAG CACTACATCA CCCCTCTGAC CTTCGGCGCT GGCACCAAGC TGGAACTGAA | 3720 |
| | GCGCACCGTC GCTATCGAGG TGATGTACCC TCCACCTTAC CTGGACAACG AGAAGAGTAA | 3780 |
| | TGGCACCATC ATCCACGTGA AGGGCAAGCA CCTGTGCCCC AGCCCCCTGT TTCCTGGCCC | 3840 |
| | CTCCAAACCC TTCTGGGTGC TGGTGGTGGT GGGCGGCGTG CTGGCCTGCT ATAGCCTGCT | 3900 |
| | GGTCACCGTG GCATTCATTA TTTTCTGGGT GTACAGAGTG GACCTGGTGC TGTTCTACAG | 3960 |
| | ACACCTGACC CGGAGAGACG AGACACTGAC AGATGGCAAA ACCTACGACG CCTTCGTGAG | 4020 |
| | CTACCTGAAG GAATGCAGAC CTGAGAACGG AGAAGAGCAC ACCTTCGCCG TGGAAATCCT | 4080 |
| | GCCCAGAGTC CTGGAAAAGC ATTTCGGCTA CAAGCTTTGC ATCTTCGAGC GGGACGTGGT | 4140 |
| | CCCCGGGGGC GCCGTGGTGG ACGAGATCCA CAGCCTGATC GAGAAGTCAC GAAGACTGAT | 4200 |
| | CATCGTGCTG AGCAAGAGCT ACATGAGCAA CGAAGTGCGG TACGAGCTGG AAAGCGGCCT | 4260 |
| | GCACGAAGCC CTTGTCGAGA GAAAGATCAA GATCATCCTG ATCGAGTTCA CCCCTGTGAC | 4320 |
| | AGATTTCACC TTCCTGCCTC AGTCACTGAA ACTGCTCAAG AGCCACAGAG TGCTGAAGTG | 4380 |
| | GAAAGCCGAT AAGTCCCTCA GCTACAACTC TCGGTTCTGG AAGAACCTGC TCTATCTGAT | 4440 |
| | GCCCGCCAAG ACTGTTAAGC CTGGCAGAGA TGAGCCTGAG GTACTCCCTG TGCTGAGCGA | 4500 |
| | GTCGGGATCT GGCGAGGGCA GAGGCAGCCT GCTGACGTGC GGCGATGTCG AGGAAAACCC | 4560 |
| | CGGTCCCATG GCCCTGCCTG TGACAGCCCT GCTGCTGCCA CTGGCCCTGC TCCTGCACGC | 4620 |
| | CGCCAGACCT CAGGTGCAGC TCCAGCAGAG CGGCCCAGAA CTCGTGCGGC CTGGAACATC | 4680 |
| | CGTGAGAATC TCGTGCAAGG CCAGTGGCTA CACCTTCACC ATCTACTGGC TGGGCTGGGT | 4740 |
| | GAAGCAAAGA CCAGGCCACG GCCTGGAATG GATCGGCAAC ATCTTCCCCG GCTCTGCCTA | 4800 |
| | CATCAACTAT AATGAGAAGT TCAAAGGCAA GGCCACACTG ACCGCCGACA CCTCTAGCTC | 4860 |
| | TACCGCCTAC ATGCAGCTGA GCAGCCTGAC AAGCGAGGAT AGCGCCGTGT ACTTCTGCGC | 4920 |
| | CCGAGAAGGC AGCAACAGCG GCTATTGGGG CCAAGGCACA ACACTCACCG TGTCTAGCGG | 4980 |
| | CGGCGGCGGC AGCGGAGGCG GTGGCTCTGG CGGCGGCGGC TCTGGCGGAG GAGGCAGCGA | 5040 |
| | TATCGTGATG ACTCAGAGCC CTAGCTCGCT AAGCGTGAGC GCCGGCGAGA AAGTGACCAT | 5100 |
| | GACCTGTAAA AGCAGCCAGA GCCTGCTGAA CAGTGGAAAT CAGCAGAACT ATCTGGCTTG | 5160 |
| | GTATCAGCAA AAGCCTGGAC AGCCTCCTAA GCTGCTGATA TACGGCGCCT CCACCAGAGA | 5220 |
| | GAGCGGTGTC CCTGACCGGT TCACAGGTTC TGGCAGCGGC ACCGACTTTA CCCTGACCAT | 5280 |
| | CAACTCCGTG CAAGCTGAAG ATCTGGCCGT GTACTACTGT CAGTCCGACC ACATCTACCC | 5340 |
| | CTACACATTC GGAGGAGGCA CCAAGCTAGA AATCAAGATC GAGGTGATGT ACCCTCCACC | 5400 |
| | ATACCTGGAC AACGAGAAGA GTAATGGCAC GATCATCCAC GTGAAGGGCA AGCACCTGTG | 5460 |
| | TCCTAGCCCC CTGTTCCCAG GCCCAAGCAA GCCTTTCTGG GTGCTGGTCG TGGTCGGAGG | 5520 |
| | CGTGCTGGCT TGTTACAGCC TGCTGGTTAC CGTGGCCTTC ATCATCTTTT GGGTGAGCGC | 5580 |
| | CCTGCTGTAC CGCCACTGGA TCGAAATCGT GCTGTTGTAC AGAACCTACC AGTCCAAGGA | 5640 |
| | CCAGACCCTT GGCGACAAGA AAGATTTTGA TGCCTTCGTG TCTTACGCTA AATGGTCCAG | 5700 |
| | CTTCCCTAGC GAAGCCACAA GTTCTCTATC TGAGGAGCAC CTGGCCCTGT CTCTGTTCCC | 5760 |
| | CGATGTGCTG GAAAACAAGT ACGGCTACAG CCTGTGCCTG CTGGAACGGG ACGTGGCCCC | 5820 |
| | TGGTGGAGTG TATGCCGAGG ACATCGTGAG CATTATCAAA AGAAGCAGAC GGGGCATCTT | 5880 |
| | TATTCTGTCT CCTAATTACG TGAACGGCCC AAGCATTTTT GAGCTGCAGG CTGCTGTGAA | 5940 |
| | CCTGGCCCTG GACGACCAGA CCCTGAAGCT GATCCTGATC AAGTTCTGCT ACTTTCAGGA | 6000 |
| | GCCTGAGAGC CTGCCGCACC TGGTGAAGAA GGCCCTGCGG GTTCTGCCAA CCGTGACCTG | 6060 |
| | GCGGGGCCTG AAGAGCGTGC CTCCTAACAG CAGGTTCTGG GCCAAGATGA GATACCACAT | 6120 |
| | GCCTGTGAAA AACTCCCAAG GCTTCACCTG GAACCAACTG AGAATCACCT CTAGAATCTT | 6180 |
| | CCAGTGGAAG GGCCTGAGCC GGACCGAGAC AACCGGCAGA TCTTCTCAGC CAAAAGAGTG | 6240 |
| | GTGATAAGAA TTCGATATCA AGCTTATCGG TAATCAACCT CTGGATTACA AAATTTGTGA | 6300 |
| | AAGATTGACT GGTATTCTTA ACTATGTTGC TCCTTTTACG CTATGTGGAT ACGCTGCTTT | 6360 |
| | AATGCCTTTG TATCATGCTA TTGCTTCCCG TATGGCTTTC ATTTTCTCCT CCTTGTATAA | 6420 |
| | ATCCTGGTTG CTGTCTCTTT ATGAGGAGTT GTGGCCCGTT GTCAGGCAAC GTGGCGTGGT | 6480 |
| | GTGCACTGTG TTTGCTGACG CAACCCCCAC TGGTTGGGGC ATTGCCACCA CCTGTCAGCT | 6540 |
| | CCTTTCCGGG ACTTTCGCTT TCCCCCTCCC TATTGCCACG GCGGAACTCA TCGCCGCCTG | 6600 |
| | CCTTGCCCGC TGCTGGACAG GGGCTCGGCT GTTGGGCACT GACAATTCCG TGGTGTTGTC | 6660 |
| | GGGGAAATCA TCGTCCTTTC CTTGGCTGCT CGCCTGTGTT GCCACCTGGA TTCTGCGCGG | 6720 |
| | GACGTCCTTC TGCTACGTCC CTTCGGCCCT CAATCCAGCG GACCTTCCTT CCCGCGGCCT | 6780 |
| | GCTGCCGGCT CTGCGGCCTC TTCCGCGTCT TCGCCTTCGC CCTCAGACGA GTCGGATCTC | 6840 |
| | CCTTTGGGCC GCCTCCCCGC ATCGATACCG TCGACCTCGA GACCTAGAAA AACATGGAGC | 6900 |
| | AATCACAAGT AGCAATACAG CAGCTACCAA TGCTGATTGT GCCTGGCTAG AAGCACAAGA | 6960 |
| | GGAGGAGGAG GTGGGTTTTC CAGTCACACC TCAGGTACCT TTAAGACCAA TGACTTACAA | 7020 |
| | GGCAGCTGTA GATCTTAGCC ACTTTTTAAA AGAAAAGGGG GGACTGGAAG GGCTAATTCA | 7080 |
| | CTCCCAACGA AGACAAGATA TCCTTGATCT GTGGATCTAC CACACACAAG GCTACTTCCC | 7140 |
| | TGATTGGCAG AACTACACAC CAGGGCCAGG GATCAGATAT CCACTGACCT TTGGATGGTG | 7200 |
| | CTACAAGCTA GTACCAGTTG AGCAAGAGAA GGTAGAAGAA GCCAATGAAG GAGAGAACAC | 7260 |
| | CCGCTTGTTA CACCCTGTGA GCCTGCATGG GATGGATGAC CCGGAGAGAG AAGTATTAGA | 7320 |
| | GTGGAGGTTT GACAGCCGCC TAGCATTTCA TCACATGGCC CGAGAGCTGC ATCCGGACTG | 7380 |
| | TACTGGGTCT CTCTGGTTAG ACCAGATCTG AGCCTGGGAG CTCTCTGGCT AACTAGGGAA | 7440 |
| | CCCACTGCTT AAGCCTCAAT AAAGCTTGCC TTGAGTGCTT CAAGTAGTGT GTGCCCGTCT | 7500 |
| | GTTGTGTGAC TCTGGTAACT AGAGATCCCT CAGACCCTTT TAGTCAGTGT GGAAAATCTC | 7560 |
| | TAGCAGGGCC CGTTTAAACC CGCTGATCAG CCTCGACTGT GCCTTCTAGT TGCCAGCCAT | 7620 |
| | CTGTTGTTTG CCCC CCCCC GTGCCTTCCT TGACCCTGGA AGGTGCCACT CCCACTGTCC | 7680 |
| | TTTCCTAATA AAATGAGGAA ATTGCATCGC ATTGTCTGAG TAGGTGTCAT TCTATTCTGG | 7740 |
| | GGGGTGGGGT GGGGCAGGAC AGCAAGGGGG AGGATTGGGA AGACAATAGC AGGCATGCTG | 7800 |
| | GGGATGCGGT GGGCTCTATG GCTTCTGAGG CGGAAAGAAC CAGCTGGGGC TCTAGGGGGT | 7860 |
| | ATCCCCACGC GCCCTGTAGC GGCGCATTAA GCGCGGCGGG TGTGGTGGTT ACGCGCAGCG | 7920 |
| | TGACCGCTAC ACTTGCCAGC GCCCTAGCGC CCGCTCCTTT CGCTTTCTTC CCTTCCTTTC | 7980 |
| | TCGCCACGTT CGCCGGCTTT CCCCGTCAAG CTCTAAATCG GGGGCTCCCT TTAGGGTTCC | 8040 |
| | GATTTAGTGC TTTACGGCAC CTCGACCCCA AAAAACTTGA TTAGGGTGAT GGTTCACGTA | 8100 |
| | GTGGGCCATC GCCCTGATAG ACGGTTTTTC GCCCTTTGAC GTTGGAGTCC ACGTTCTTTA | 8160 |
| | ATAGTGGACT CTTGETCCAA ACTGGAACAA CACTCAACCC TATCTCGGTC TATTCTTTTG | 8220 |
| | ATTTATAAGG GATTTTGCCG ATTTCGGCCT ATTGGTTAAA AAATGAGCTG ATTTAACAAA | 8280 |
| | AATTTAACGC GAATTAATTC TGTGGAATGT GTGTCAGTTA GGGTGTGGAA AGTCCCCAGG | 8340 |
| | CTCCCCAGCA GGCAGAAGTA TGCAAAGCAT GCATCTCAAT TAGTCAGCAA CCAGGTGTGG | 8400 |
| | AAAGTCCCCA GGCTCCCCAG CAGGCAGAAG TATGCAAAGC ATGCATCTCA ATTAGTCAGC | 8460 |
| | AACCATAGTC CCGCCCCTAA CTCCGCCCAT CCCGCCCCTA ACTCCGCCCA GTTCCGCCCA | 8520 |
| | TTCTCCGCCC CATGGCTGAC TAATTTTTTT TATTTATGCA GAGGCCGAGG CCGCCTCTGC | 8580 |
| | CTCTGAGCTA TTCCAGAAGT AGTGAGGAGG CTTTTTTGGA GGCCTAGGCT TTTGCAAAAA | 8640 |
| | GCTCCCGGGA GCTTGTATAT CCATTTTCGG ATCTGATCAG CACGTGTTGA CAATTAATCA | 8700 |
| | TCGGCATAGT ATATCGGCAT AGTATAATAC GACAAGGTGA GGAACTAAAC CATGGCCAAG | 8760 |
| | TTGACCAGTG CCGTTCCGGT GCTCACCGCG CGCGACGTCG CCGGAGCGGT CGAGTTCTGG | 8820 |
| | ACCGACCGGC TCGGGTTCTC CCGGGACTTC GTGGAGGACG ACTTCGCCGG TGTGGTCCGG | 8880 |
| | GACGACGTGA CCCTGTTCAT CAGCGCGGTC CAGGACCAGG TGGTGCCGGA CAACACCCTG | 8940 |
| | GCCTGGGTGT GGGTGCGCGG CCTGGACGAG CTGTACGCCG AGTGGTCGGA GGTCGTGTCC | 9000 |
| | ACGAACTTCC GGGACGCCTC CGGGCCGGCC ATGACCGAGA TCGGCGAGCA GCCGTGGGGG | 9060 |
| | CGGGAGTTCG CCCTGCGCGA CCCGGCCGGC AACTGCGTGC ACTTCGTGGC CGAGGAGCAG | 9120 |
| | GACTGACACG TGCTACGAGA TTTCGATTCC ACCGCCGCCT TCTATGAAAG GTTGGGCTTC | 9180 |
| | GGAATCGTTT TCCGGGACGC CGGCTGGATG ATCCTCCAGC GCGGGGATCT CATGCTGGAG | 9240 |
| | TTCTTCGCCC ACCCCAACTT GTTTATTGCA GCTTATAATG GTTACAAATA AAGCAATAGC | 9300 |
| | ATCACAAATT TCACAAATAA AGCATTTTTT TCACTGCATT CTAGTTGTGG TTTGTCCAAA | 9360 |
| | CTCATCAATG TATCTTATCA TGTCTGTATA CCGTCGACCT CTAGCTAGAG CTTGGCGTAA | 9420 |
| | TCATGGTCAT AGCTGTTTCC TGTGTGAAAT TGTTATCCGC TCACAATTCC ACACAACATA | 9480 |
| | CGAGCCGGAA GCATAAAGTG TAAAGCCTGG GGTGCCTAAT GAGTGAGCTA ACTCACATTA | 9540 |
| | ATTGCGTTGC GCTCACTGCC CGCTTTCCAG TCGGGAAACC TGTCGTGCCA GCTGCATTAA | 9600 |
| | TGAATCGGCC AACGCGCGGG GAGAGGCGGT TTGCGTATTG GGCGCTCTTC CGCTTCCTCG | 9660 |
| | CTCACTGACT CGCTGCGCTC GGTCGTTCGG CTGCGGCGAG CGGTATCAGC TCACTCAAAG | 9720 |
| | GCGGTAATAC GGTTATCCAC AGAATCAGGG GATAACGCAG GAAAGAACAT GTGAGCAAAA | 9780 |
| | GGCCAGCAAA AGGCCAGGAA CCGTAAAAAG GCCGCGTTGC TGGCGTTTTT CCATAGGCTC | 9840 |
| | CGCCCCCCTG ACGAGCATCA CAAAAATCGA CGCTCAAGTC AGAGGTGGCG AAACCCGACA | 9900 |
| | GGACTATAAA GATACCAGGC GTTTCCCCCT GGAAGCTCCC TCGTGCGCTC TCCTGTTCCG | 9960 |
| | ACCCTGCCGC TTACCGGATA CCTGTCCGCC TTTCTCCCTT CGGGAAGCGT GGCGCTTTCT | 10020 |
| | CATAGCTCAC GCTGTAGGTA TCTCAGTTCG GTGTAGGTCG TTCGCTCCAA GCTGGGCTGT | 10080 |
| | GTGCACGAAC CCCCCGTTCA GCCCGACCGC TGCGCCTTAT CCGGTAACTA TCGTCTTGAG | 10140 |
| | TCCAACCCGG TAAGACACGA CTTATCGCCA CTGGCAGCAG CCACTGGTAA CAGGATTAGC | 10200 |
| | AGAGCGAGGT ATGTAGGCGG TGCTACAGAG TTCTTGAAGT GGTGGCCTAA CTACGGCTAC | 10260 |
| | ACTAGAAGAA CAGTATTTGG TATCTGCGCT CTGCTGAAGC CAGTTACCTT CGGAAAAAGA | 10320 |
| | GTTGGTAGCT CTTGATCCGG CAAACAAACC ACCGCTGGTA GCGGTGGTTT TTTTGTTTGC | 10380 |
| | AAGCAGCAGA TTACGCGCAG AAAAAAAGGA TCTCAAGAAG ATCCTTTGAT CTTTTCTACG | 10440 |
| | GGGTCTGACG CTCAGTGGAA CGAAAACTCA CGTTAAGGGA TTTTGGTCAT GAGATTATCA | 10500 |
| | AAAAGGATCT TCACCTAGAT CCTTTTAAAT TAAAAATGAA GTTTTAAATC AATCTAAAGT | 10560 |
| | ATATATGAGT AAACTTGGTC TGACAGTTAC CAATGCTTAA TCAGTGAGGC ACCTATCTCA | 10620 |
| | GCGATCTGTC TATTTCGTTC ATCCATAGTT GCCTGACTCC CCGTCGTGTA GATAACTACG | 10680 |
| | ATACGGGAGG GCTTACCATC TGGCCCCAGT GCTGCAATGA TACCGCGAGA CCCACGCTCA | 10740 |
| | CCGGCTCCAG ATTTATCAGC AATAAACCAG CCAGCCGGAA GGGCCGAGCG CAGAAGTGGT | 10800 |
| | CCTGCAACTT TATCCGCCTC CATCCAGTCT ATTAATTGTT GCCGGGAAGC TAGAGTAAGT | 10860 |
| | AGTTCGCCAG TTAATAGTTT GCGCAACGTT GTTGCCATTG CTACAGGCAT CGTGGTGTCA | 10920 |
| | CGCTCGTCGT TTGGTATGGC TTCATTCAGC TCCGGTTCCC AACGATCAAG GCGAGTTACA | 10980 |
| | TGATCCCCCA TGTTGTGCAA AAAAGCGGTT AGCTCCTTCG GTCCTCCGAT CGTTGTCAGA | 11040 |
| | AGTAAGTTGG CCGCAGTGTT ATCACTCATG GTTATGGCAG CACTGCATAA TTCTCTTACT | 11100 |
| | GTCATGCCAT CCGTAAGATG CTTTTCTGTG ACTGGTGAGT ACTCAACCAA GTCATTCTGA | 11160 |
| | GAATAGTGTA TGCGGCGACC GAGTTGCTCT TGCCCGGCGT CAATACGGGA TAATACCGCG | 11220 |
| | CCACATAGCA GAACTTTAAA AGTGCTCATC ATTGGAAAAC GTTCTTCGGG GCGAAAACTC | 11280 |
| | TCAAGGATCT TACCGCTGTT GAGATCCAGT TCGATGTAAC CCACTCGTGC ACCCAACTGA | 11340 |
| | TCTTCAGCAT CTTTTACTTT CACCAGCGTT TCTGGGTGAG CAAAAACAGG AAGGCAAAAT | 11400 |
| | GCCGCAAAAA AGGGAATAAG GGCGACACGG AAATGTTGAA TACTCATACT CTTCCTTTTT | 11460 |
| | CAATATTATT GAAGCATTTA TCAGGGTTAT TGTCTCATGA GCGGATACAT ATTTGAATGT | 11520 |
| | ATTTAGAAAA ATAAACAAAT AGGGGTTCCG CGCACATTTC CCCGAAAAGT GCCACCTGAC | 11580 |

In an embodiment, a CCR comprises an anti-TROP-2-V_{L}-linker-anti-TROP-2-V_{H}-IgG4 (hinge and transmembrane)-IL2Rβ construct. In an embodiment, a CCR is encoded by a nucleotide sequence comprising SEQ ID NO:618. In an embodiment, a CCR is encoded by a nucleotide sequence comprising a region that is at least 99% identical to SEQ ID NO:618. In an embodiment, a CCR is encoded by a nucleotide sequence comprising a region that is at least 98% identical to SEQ ID NO:618. In an embodiment, a CCR is encoded by a nucleotide sequence comprising a region that is at least 97% identical to SEQ ID NO:618. In an embodiment, a CCR is encoded by a nucleotide sequence comprising a region that is at least 96% identical to SEQ ID NO:618. In an embodiment, a CCR is encoded by a nucleotide sequence comprising a region that is at least 95% identical to SEQ ID NO:618. In an embodiment, a CCR is encoded by a nucleotide sequence comprising a region that is at least 94% identical to SEQ ID NO:618. In an embodiment, a CCR is encoded by a nucleotide sequence comprising a region that is at least 93% identical to SEQ ID NO:618. In an embodiment, a CCR is encoded by a nucleotide sequence comprising a region that is at least 92% identical to SEQ ID NO:618. In an embodiment, a CCR is encoded by a nucleotide sequence comprising a region that is at least 91% identical to SEQ ID NO:618. In an embodiment, a CCR is encoded by a nucleotide sequence comprising a region that is at least 90% identical to SEQ ID NO:618. In an embodiment, a CCR is encoded by a nucleotide sequence comprising a region that is at least 85% identical to SEQ ID NO:618. In an embodiment, a CCR is encoded by a nucleotide sequence comprising a region that is at least 80% identical to SEQ ID NO:618.

In an embodiment, a CCR comprises an anti-FAP-V_{L}-linker-anti-FAP-V_{H}-CD8α (hinge and transmembrane)-IL-18R1 construct. In an embodiment, a CCR is encoded by a nucleotide sequence comprising SEQ ID NO:619. In an embodiment, a CCR is encoded by a nucleotide sequence comprising a region that is at least 99% identical to SEQ ID NO:619. In an embodiment, a CCR is encoded by a nucleotide sequence comprising a region that is at least 98% identical to SEQ ID NO:619. In an embodiment, a CCR is encoded by a nucleotide sequence comprising a region that is at least 97% identical to SEQ ID NO:619. In an embodiment, a CCR is encoded by a nucleotide sequence comprising a region that is at least 96% identical to SEQ ID NO:619. In an embodiment, a CCR is encoded by a nucleotide sequence comprising a region that is at least 95% identical to SEQ ID NO:619. In an embodiment, a CCR is encoded by a nucleotide sequence comprising a region that is at least 94% identical to SEQ ID NO:619. In an embodiment, a CCR is encoded by a nucleotide sequence comprising a region that is at least 93% identical to SEQ ID NO:619. In an embodiment, a CCR is encoded by a nucleotide sequence comprising a region that is at least 92% identical to SEQ ID NO:619. In an embodiment, a CCR is encoded by a nucleotide sequence comprising a region that is at least 91% identical to SEQ ID NO:619. In an embodiment, a CCR is encoded by a nucleotide sequence comprising a region that is at least 90% identical to SEQ ID NO:619. In an embodiment, a CCR is encoded by a nucleotide sequence comprising a region that is at least 85% identical to SEQ ID NO:619. In an embodiment, a CCR is encoded by a nucleotide sequence comprising a region that is at least 80% identical to SEQ ID NO:619.

In an embodiment, a CCR comprises an anti-PD-L1-V_{L}-linker-anti-PD-L1-V_{H}-CD8α (hinge and transmembrane)-CD27 construct, based on the 38A1 antibody described herein. In an embodiment, a CCR is encoded by a nucleotide sequence comprising SEQ ID NO:620. In an embodiment, a CCR is encoded by a nucleotide sequence comprising a region that is at least 99% identical to SEQ ID NO:620. In an embodiment, a CCR is encoded by a nucleotide sequence comprising a region that is at least 98% identical to SEQ ID NO:620. In an embodiment, a CCR is encoded by a nucleotide sequence comprising a region that is at least 97% identical to SEQ ID NO:620. In an embodiment, a CCR is encoded by a nucleotide sequence comprising a region that is at least 96% identical to SEQ ID NO:620. In an embodiment, a CCR is encoded by a nucleotide sequence comprising a region that is at least 95% identical to SEQ ID NO:620. In an embodiment, a CCR is encoded by a nucleotide sequence comprising a region that is at least 94% identical to SEQ ID NO:620. In an embodiment, a CCR is encoded by a nucleotide sequence comprising a region that is at least 93% identical to SEQ ID NO:620. In an embodiment, a CCR is encoded by a nucleotide sequence comprising a region that is at least 92% identical to SEQ ID NO:620. In an embodiment, a CCR is encoded by a nucleotide sequence comprising a region that is at least 910% identical to SEQ ID NO:620. In an embodiment, a CCR is encoded by a nucleotide sequence comprising a region that is at least 90% identical to SEQ ID NO:620. In an embodiment, a CCR is encoded by a nucleotide sequence comprising a region that is at least 85% identical to SEQ ID NO:620. In an embodiment, a CCR is encoded by a nucleotide sequence comprising a region that is at least 80% identical to SEQ ID NO:620.

In an embodiment, a biepitope CCR comprises two CCRs comprising SP-(38A1 scFv)-(CD28 hinge and transmembrane)-(IL-2Rβ intracellular)-T2A-SP-(19H9 scFv)-(CD28 hinge and transmembrane)-(IL-2Rγ intracellular), using both the 38A1 and 19H9 PD-L1 domains described herein, wherein SP refers to a signal peptide. In an embodiment, a biepitope CCR is encoded by a nucleotide sequence comprising SEQ ID NO:621. In an embodiment, a biepitope CCR is encoded by a nucleotide sequence comprising a region that is at least 99% identical to SEQ ID NO:621. In an embodiment, a biepitope CCR is encoded by a nucleotide sequence comprising a region that is at least 98% identical to SEQ ID NO:621. In an embodiment, a biepitope CCR is encoded by a nucleotide sequence comprising a region that is at least 97% identical to SEQ ID NO:621. In an embodiment, a biepitope CCR is encoded by a nucleotide sequence comprising a region that is at least 96% identical to SEQ ID NO:621. In an embodiment, a biepitope CCR is encoded by a nucleotide sequence comprising a region that is at least 95% identical to SEQ ID NO:621. In an embodiment, a biepitope CCR is encoded by a nucleotide sequence comprising a region that is at least 94% identical to SEQ ID NO:621. In an embodiment, a biepitope CCR is encoded by a nucleotide sequence comprising a region that is at least 93% identical to SEQ ID NO:621. In an embodiment, a biepitope CCR is encoded by a nucleotide sequence comprising a region that is at least 92% identical to SEQ ID NO:621. In an embodiment, a biepitope CCR is encoded by a nucleotide sequence comprising a region that is at least 91% identical to SEQ ID NO:621. In an embodiment, a biepitope CCR is encoded by a nucleotide sequence comprising a region that is at least 90% identical to SEQ ID NO:621. In an embodiment, a biepitope CCR is encoded by a nucleotide sequence comprising a region that is at least 85% identical to SEQ ID NO:621. In an embodiment, a biepitope CCR is encoded by a nucleotide sequence comprising a region that is at least 80% identical to SEQ ID NO:621. The foregoing embodiments may include or may omit a fluorescent protein, such as eGFP, for analytical use as needed.

In an embodiment, a biepitope CCR comprises two CCRs comprising SP-(38A1 scFv)-(CD28 hinge and transmembrane)-(IL-18R1 intracellular)-T2A-SP-(19H9 scFv)-(CD28 hinge and transmembrane)-(IL-18RAP intracellular), using both the 38A1 and 19H9 PD-L1 domains described herein, wherein SP refers to a signal peptide. In an embodiment, a biepitope CCR is encoded by a nucleotide sequence comprising SEQ ID NO:622. In an embodiment, a biepitope CCR is encoded by a nucleotide sequence comprising a region that is at least 99% identical to SEQ ID NO:622. In an embodiment, a biepitope CCR is encoded by a nucleotide sequence comprising a region that is at least 98% identical to SEQ ID NO:622. In an embodiment, a biepitope CCR is encoded by a nucleotide sequence comprising a region that is at least 97% identical to SEQ ID NO:622. In an embodiment, a biepitope CCR is encoded by a nucleotide sequence comprising a region that is at least 96% identical to SEQ ID NO:622. In an embodiment, a biepitope CCR is encoded by a nucleotide sequence comprising a region that is at least 95% identical to SEQ ID NO:622. In an embodiment, a biepitope CCR is encoded by a nucleotide sequence comprising a region that is at least 94% identical to SEQ ID NO:622. In an embodiment, a biepitope CCR is encoded by a nucleotide sequence comprising a region that is at least 93% identical to SEQ ID NO:622. In an embodiment, a biepitope CCR is encoded by a nucleotide sequence comprising a region that is at least 92% identical to SEQ ID NO:622. In an embodiment, a biepitope CCR is encoded by a nucleotide sequence comprising a region that is at least 91% identical to SEQ ID NO:622. In an embodiment, a biepitope CCR is encoded by a nucleotide sequence comprising a region that is at least 90% identical to SEQ ID NO:622. In an embodiment, a biepitope CCR is encoded by a nucleotide sequence comprising a region that is at least 85% identical to SEQ ID NO:622. In an embodiment, a biepitope CCR is encoded by a nucleotide sequence comprising a region that is at least 80% identical to SEQ ID NO:622. The foregoing embodiments may include or may omit a fluorescent protein, such as eGFP, for analytical use as needed.

In an embodiment, a biepitope CCR comprises two CCRs comprising SP-(anti-TROP-2 scFv)-(CD8 hinge)-(IL-2Rβ transmembrane and intracellular)-T2A-SP-(anti-TROP-2 scFv)-(CD8 hinge)-(IL-2Rγ transmembrane and intracellular), wherein SP refers to a signal peptide. In an embodiment, a biepitope CCR is encoded by a nucleotide sequence comprising SEQ ID NO:622. In an embodiment, a biepitope CCR is encoded by a nucleotide sequence comprising a region that is at least 99% identical to SEQ ID NO:623. In an embodiment, a biepitope CCR is encoded by a nucleotide sequence comprising a region that is at least 98% identical to SEQ ID NO:623. In an embodiment, a biepitope CCR is encoded by a nucleotide sequence comprising a region that is at least 97% identical to SEQ ID NO:623. In an embodiment, a biepitope CCR is encoded by a nucleotide sequence comprising a region that is at least 96% identical to SEQ ID NO:623. In an embodiment, a biepitope CCR is encoded by a nucleotide sequence comprising a region that is at least 95% identical to SEQ ID NO:623. In an embodiment, a biepitope CCR is encoded by a nucleotide sequence comprising a region that is at least 94% identical to SEQ ID NO:623. In an embodiment, a biepitope CCR is encoded by a nucleotide sequence comprising a region that is at least 93% identical to SEQ ID NO:623. In an embodiment, a biepitope CCR is encoded by a nucleotide sequence comprising a region that is at least 92% identical to SEQ ID NO:623. In an embodiment, a biepitope CCR is encoded by a nucleotide sequence comprising a region that is at least 91% identical to SEQ ID NO:623. In an embodiment, a biepitope CCR is encoded by a nucleotide sequence comprising a region that is at least 90% identical to SEQ ID NO:623. In an embodiment, a biepitope CCR is encoded by a nucleotide sequence comprising a region that is at least 85% identical to SEQ ID NO:623. In an embodiment, a biepitope CCR is encoded by a nucleotide sequence comprising a region that is at least 80% identical to SEQ ID NO:623. The foregoing embodiments may include or may omit a fluorescent protein, such as eGFP, for analytical use as needed.

In an embodiment, a biepitope CCR comprises two CCRs comprising SP-(anti-TROP-2 scFv)-(CD8 hinge)-(IL-18R1-transmembrane and intracellular)-T2A-SP-(anti-TROP-2 scFv)-(CD8 hinge)-(IL-18RAP-transmembrane and intracellular), wherein SP refers to a signal peptide. In an embodiment, a biepitope CCR is encoded by a nucleotide sequence comprising SEQ ID NO:624. In an embodiment, a biepitope CCR is encoded by a nucleotide sequence comprising a region that is at least 99% identical to SEQ ID NO:624. In an embodiment, a biepitope CCR is encoded by a nucleotide sequence comprising a region that is at least 98% identical to SEQ ID NO:624. In an embodiment, a biepitope CCR is encoded by a nucleotide sequence comprising a region that is at least 97% identical to SEQ ID NO:624. In an embodiment, a biepitope CCR is encoded by a nucleotide sequence comprising a region that is at least 96% identical to SEQ ID NO:624. In an embodiment, a biepitope CCR is encoded by a nucleotide sequence comprising a region that is at least 95% identical to SEQ ID NO:624. In an embodiment, a biepitope CCR is encoded by a nucleotide sequence comprising a region that is at least 94% identical to SEQ ID NO:624. In an embodiment, a biepitope CCR is encoded by a nucleotide sequence comprising a region that is at least 93% identical to SEQ ID NO:624. In an embodiment, a biepitope CCR is encoded by a nucleotide sequence comprising a region that is at least 92% identical to SEQ ID NO:624. In an embodiment, a biepitope CCR is encoded by a nucleotide sequence comprising a region that is at least 91% identical to SEQ ID NO:624. In an embodiment, a biepitope CCR is encoded by a nucleotide sequence comprising a region that is at least 90% identical to SEQ ID NO:624. In an embodiment, a biepitope CCR is encoded by a nucleotide sequence comprising a region that is at least 85% identical to SEQ ID NO:624. In an embodiment, a biepitope CCR is encoded by a nucleotide sequence comprising a region that is at least 80% identical to SEQ ID NO:624. The foregoing embodiments may include or may omit a fluorescent protein, such as eGFP, for analytical use as needed.

In an embodiment, a biepitope CCR comprises two CCRs comprising SP-(cAR47A6.4 scFv)-(CD28 hinge-transmembrane)-(IL-2Rβ intracellular)-T2A-SP-(KM4097 scFv)-(CD28 hinge and transmembrane)-(IL-2Rγ intracellular), wherein SP refers to a signal peptide. In an embodiment, a biepitope CCR is encoded by a nucleotide sequence comprising SEQ ID NO:625. In an embodiment, a biepitope CCR is encoded by a nucleotide sequence comprising a region that is at least 99% identical to SEQ ID NO:625. In an embodiment, a biepitope CCR is encoded by a nucleotide sequence comprising a region that is at least 98% identical to SEQ ID NO:625. In an embodiment, a biepitope CCR is encoded by a nucleotide sequence comprising a region that is at least 97% identical to SEQ ID NO:625. In an embodiment, a biepitope CCR is encoded by a nucleotide sequence comprising a region that is at least 96% identical to SEQ ID NO:625. In an embodiment, a biepitope CCR is encoded by a nucleotide sequence comprising a region that is at least 95% identical to SEQ ID NO:625. In an embodiment, a biepitope CCR is encoded by a nucleotide sequence comprising a region that is at least 94% identical to SEQ ID NO:625. In an embodiment, a biepitope CCR is encoded by a nucleotide sequence comprising a region that is at least 93% identical to SEQ ID NO:625. In an embodiment, a biepitope CCR is encoded by a nucleotide sequence comprising a region that is at least 92% identical to SEQ ID NO:625. In an embodiment, a biepitope CCR is encoded by a nucleotide sequence comprising a region that is at least 91% identical to SEQ ID NO:625. In an embodiment, a biepitope CCR is encoded by a nucleotide sequence comprising a region that is at least 90% identical to SEQ ID NO:625. In an embodiment, a biepitope CCR is encoded by a nucleotide sequence comprising a region that is at least 85% identical to SEQ ID NO:625. In an embodiment, a biepitope CCR is encoded by a nucleotide sequence comprising a region that is at least 80% identical to SEQ ID NO:625. The foregoing embodiments may include or may omit a fluorescent protein, such as eGFP, for analytical use as needed.

In an embodiment, a biepitope CCR comprises two CCRs comprising SP-(cAR47A6.4 scFv)-(CD28 hinge-transmembrane)-(IL-18R1 intracellular)-T2A-SP-(KM4097scFv)-(CD28 hinge-transmembrane)-(IL-18RAP intracellular), wherein SP refers to a signal peptide. In an embodiment, a biepitope CCR is encoded by a nucleotide sequence comprising SEQ ID NO:626. In an embodiment, a biepitope CCR is encoded by a nucleotide sequence comprising a region that is at least 99% identical to SEQ ID NO:626. In an embodiment, a biepitope CCR is encoded by a nucleotide sequence comprising a region that is at least 98% identical to SEQ ID NO:626. In an embodiment, a biepitope CCR is encoded by a nucleotide sequence comprising a region that is at least 97% identical to SEQ ID NO:626. In an embodiment, a biepitope CCR is encoded by a nucleotide sequence comprising a region that is at least 96% identical to SEQ ID NO:626. In an embodiment, a biepitope CCR is encoded by a nucleotide sequence comprising a region that is at least 95% identical to SEQ ID NO:626. In an embodiment, a biepitope CCR is encoded by a nucleotide sequence comprising a region that is at least 94% identical to SEQ ID NO:626. In an embodiment, a biepitope CCR is encoded by a nucleotide sequence comprising a region that is at least 93% identical to SEQ ID NO:626. In an embodiment, a biepitope CCR is encoded by a nucleotide sequence comprising a region that is at least 92% identical to SEQ ID NO:626. In an embodiment, a biepitope CCR is encoded by a nucleotide sequence comprising a region that is at least 91% identical to SEQ ID NO:626. In an embodiment, a biepitope CCR is encoded by a nucleotide sequence comprising a region that is at least 90% identical to SEQ ID NO:626. In an embodiment, a biepitope CCR is encoded by a nucleotide sequence comprising a region that is at least 85% identical to SEQ ID NO:626. In an embodiment, a biepitope CCR is encoded by a nucleotide sequence comprising a region that is at least 80% identical to SEQ ID NO:626. The foregoing embodiments may include or may omit a fluorescent protein, such as eGFP, for analytical use as needed.

In an embodiment, a CCR of the present invention comprises the amino acid sequence of SEQ ID NO:658, SEQ ID NO:659, SEQ ID NO:660, SEQ ID NO:661, SEQ ID NO:662, or SEQ ID NO:663, or conservative amino acid substitutions thereof, or fragments, variants, or derivatives thereof, or an amino acid sequence that is at least 99% identical to the sequence given in SEQ ID NO:658, SEQ ID NO:659, SEQ ID NO:660, SEQ ID NO:661, SEQ ID NO:662, or SEQ ID NO:663, at least 98% identical to the sequence given in SEQ ID NO:658, SEQ ID NO:659, SEQ ID NO:660, SEQ ID NO:661, SEQ ID NO:662, or SEQ ID NO:663, at least 97% identical to the sequence given in SEQ ID NO:562, at least 96% identical to the sequence given in SEQ ID NO:658, SEQ ID NO:659, SEQ ID NO:660, SEQ ID NO:661, SEQ ID NO:662, or SEQ ID NO:663, at least 95% identical to the sequence given in SEQ ID NO:658, SEQ ID NO:659, SEQ ID NO:660, SEQ ID NO:661, SEQ ID NO:662, or SEQ ID NO:663, at least 90% identical to the sequence given in SEQ ID NO:562, at least 85% identical to the sequence given in SEQ ID NO:658. SEQ ID NO:659, SEQ ID NO:660, SEQ ID NO:661, SEQ ID NO:662, or SEQ ID NO:663, or at least 80% identical to the sequence given in SEQ ID NO:658, SEQ ID NO:659, SEQ ID NO:660, SEQ ID NO:661, SEQ ID NO:662, or SEQ ID NO:663.

In an embodiment, more than one CCR is encoded by multiple transgenes in a polycistronic vector. In an embodiment, at least one chemokine receptor and at least one CCR are encoded by multiple transgenes in a polycistronic vector. In an embodiment, at least two chemokine receptors and at least one CCR are encoded by multiple transgenes in a polycistronic vector. In an embodiment, at least one chemokine receptor and at least two CCRs are encoded by multiple transgenes in a polycistronic vector. In any of the foregoing embodiments, the CCRs and/or chemokine receptors are encoded by a bicistronic vector. Suitable polycistronic vectors are described herein and in Liu, et al., Scientific Reports 2017, 7(1), 2193; Kim, et al., PLoS One 2011, 6(4), e18556. the disclosures of which are incorporated herein by reference. The IRES technique may also be employed in embodiments herein to achieve polycistronic vector designs.

In an embodiment, a CCR of the present invention is a biepitope CCR, comprising two CCRs that bind to different epitopes of the same target. In an embodiment, two CCRs are encoded by a bicistronic vector, wherein each CCR binds to a different epitope of a target. In an embodiment, two CCRs are encoded by a bicistronic vector, wherein the first CCR comprises a first scFv domain that binds to a first epitope of a target selected from the group consisting of CD19, CD20, CD22, CD24, CD33, CD38, CD39, CD73, CD123, CD138, CD228, LRRC15, CEA, FRα, EPCAM (CD326), DLL3, PD-1, PD-L1 (CD274), PSMA, gp100, MUC1, MCSP, EGFR, GD2, TROP-2, GPC3, MICA, MICB, VISTA, ULBP, HER2, MCM5, FAP, 5T4, LFA-1, B7-H3, and MUC16, and the second CCR comprises a second scFv domain that binds to a different epitope of the target. In an embodiment, two CCRs are encoded by a bicistronic vector, wherein the first CCR comprises an scFv domain that binds to a first epitope of PD-L1 and the second CCR comprises an scFv domain that binds to a second epitope of PD-L1. In an embodiment, two CCRs are encoded by a bicistronic vector, wherein the first CCR comprises an scFv domain that binds to a first epitope of PD-1 and the second CCR comprises an scFv domain that binds to a second epitope of PD-1. In an embodiment, two CCRs are encoded by a bicistronic vector, wherein the first CCR comprises an scFv domain that binds to a first epitope of CEA and the second CCR comprises an scFv domain that binds to a second epitope of CEA. In an embodiment, two CCRs are encoded by a bicistronic vector, wherein the first CCR comprises an scFv domain that binds to a first epitope of CD73 and the second CCR comprises an scFv domain that binds to a second epitope of CD73. In an embodiment, two CCRs are encoded by a bicistronic vector, wherein the first CCR comprises an scFv domain that binds to a first epitope of TROP-2 and the second CCR comprises an scFv domain that binds to a second epitope of TROP-2. In an embodiment, two CCRs are encoded by a bicistronic vector, wherein the first CCR comprises an scFv domain that binds to a first epitope of tissue factor and the second CCR comprises an scFv domain that binds to a second epitope of tissue factor. In an embodiment, two CCRs are encoded by a bicistronic vector, wherein the first CCR comprises an scFv domain that binds to a first epitope of LFA-1 and the second CCR comprises an scFv domain that binds to a second epitope of LFA-1. In an embodiment, two CCRs are encoded by a bicistronic vector, wherein the first CCR comprises an scFv domain that binds to a first epitope of FAP and the second CCR comprises an scFv domain that binds to a second epitope of FAP. In an embodiment, two CCRs are encoded by a bicistronic vector, wherein the first CCR comprises an scFv domain that binds to a first epitope of VISTA and the second CCR comprises an scFv domain that binds to a second epitope of VISTA. In an embodiment, two CCRs are encoded by a bicistronic vector, wherein the first CCR comprises an scFv domain that binds to a first epitope of LRRC15 and the second CCR comprises an scFv domain that binds to a second epitope of LRRC15. In an embodiment, two CCRs are encoded by a bicistronic vector, wherein the first CCR comprises an scFv domain that binds to a first epitope of DLL3 and the second CCR comprises an scFv domain that binds to a second epitope of DLL3.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein each extracellular domain comprises an scFv binding domain.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target protein to form a biepitope complex and each intracellular domain is selected to provide two subunits for signaling, wherein each extracellular domain comprises an scFv binding domain, wherein the scFv binding domain binds to an epitope of the target protein selected from the group consisting of CD19, CD20, CD22, CD24, CD33, CD38, CD39, CD73, CD123, CD138, CD228, LRRC15, CEA, FRα, EPCAM, DLL3, PD-L1, PSMA, gp100, MUC1, MCSP, EGFR, GD2, TROP-2, GPC3, MICA, MICB, VISTA, ULBP, HER2, MCM5, FAP, 5T4, LFA-1, B7-H3, IL-13Rα2, FAS, TGFβRII, and MUC16.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target protein to form a biepitope complex and each intracellular domain is selected to provide two subunits for signaling, wherein the extracellular domain is selected from the group consisting of a PD-1 domain, a FAS domain, and a TGFβRII domain.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) c) A transmembrane domain, and
(d) At least one intracellular domain;
   wherein each extracellular domain binds to a different epitope of a target protein to form a biepitope complex and each intracellular domain is selected to provide two subunits for signaling, wherein each extracellular domain comprises an scFv binding domain, wherein the scFv binding domain binds to an epitope of the target protein selected from the group consisting of CD19, CD20, CD22, CD24, CD33, CD38, CD39, CD73, CD123, CD138, CD228, LRRC15, CEA, FRα, EPCAM, DLL3, PD-L1, PSMA, gp100, MUC1, MCSP, EGFR, GD2, TROP-2, GPC3, MICA, MICB, VISTA, ULBP, HER2, MCM5, FAP, 5T4, LFA-1, B7-H3, IL-13Rα2, FAS, TGFβRII, and MUC16, and wherein the intracellular domain is selected from the group consisting of CD28, CD134 (OX40), CD278 (ICOS), CD137 (4-1BB), CD27, CD40L, STAT3, IL-2Rβ, IL-2Rγ, IL-18R1, IL-18RAP, IL-7Rα, IL-12R1, IL-12R2, IL-15Rα, IL-21R, LTBR, and combinations thereof.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein each extracellular domain comprises an scFv binding domain, wherein the scFv binding domains bind to two different epitopes of PD-L1.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein each extracellular domain comprises an scFv binding domain, wherein the scFv binding domains bind to two different epitopes of TROP-2.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein each extracellular domain comprises an scFv binding domain, wherein the scFv binding domains bind to two different epitopes of PD-L1 on each CCR, and wherein the intracellular domains are IL-18R1 and IL-18RAP on each CCR.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein each extracellular domain comprises an scFv binding domain, wherein the scFv binding domains bind to two different epitopes of TROP-2 on each CCR, and wherein the intracellular domains are IL-18R1 and IL-18RAP on each CCR.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein each extracellular domain comprises an scFv binding domain, wherein the scFv binding domains bind to two different epitopes of PD-L1 on each CCR, and wherein the intracellular domains are IL-2Rβ and IL-2γ on each CCR.

In an embodiment, the invention includes a method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express two chimeric costimulatory receptors (CCR), wherein each CCR comprises:
(a) An extracellular domain,
(b) A hinge domain,
(c) A transmembrane domain, and
(d) At least one intracellular domain;
   wherein each extracellular domain binds to a different epitope of a target antigen to form a biepitope complex and each intracellular domain is selected to provide two subunits for signaling, wherein each extracellular domain comprises an scFv binding domain, wherein the scFv binding domains bind to two different epitopes of TROP-2 on each CCR, and wherein the intracellular domains are IL-2Rβ and IL-2γ on each CCR.

### IX. Chemokine Receptors

In some embodiments, the foregoing manufacturing processes, including Gen 2 and Gen 3 and other processes of making TILs, MILs, and PBLs, may be modified to include a step comprising the viral or non-viral transduction of TILs, MILs, or PBLs to express one or more chemokine receptors, also known as chemoattractant cytokine receptors. Chemokine receptors generally have a rhodopsin-like 7-transmembrane (7TM) structure, interact with chemokines, transduce intracellular signals through coupling with G-protein, and mediate chemotaxis, as described in Murdoch and Finn, Blood 2000, 95, 3032-3043, the disclosures of which are incorporated by reference herein. In an embodiment, a TIL, MIL, or PBL is modified to express a chemokine receptor. In an embodiment, a TIL, MIL, or PBL is modified to express a chemokine receptor and a CCR. In an embodiment, a TIL, MIL, or PBL is modified to express a chemokine receptor and a CCR, such modifications occurring separately. In an embodiment, a TIL, MIL, or PBL is modified to express a chemokine receptor and a CCR, such modifications occurring simultaneously. In an embodiment, a TIL, MIL, or PBL is modified to express a chemokine receptor without also being modified to express a CCR. The chemokine receptors described in can be used to genetically modified TILs, MILs, and PBLs in conjunction with the CCRs described herein or separately from the CCRs described herein.

### A. Chemokine Receptor Domains

In an embodiment, a TIL, MIL, or PBL is transiently or stably modified to express a C-X-C (or CXC) motif chemokine receptor, such as CXCR1, CXCR2, CXCR3, CXCR4, or CXCR5. In an embodiment, a TIL, MIL, or PBL is transiently or stably modified to express a C-C motif chemokine receptor. Suitable C-C motif chemokine receptors are CCR2, CCR4, CCR6, CCR7, and CCR8. The designation “CCR” used in combination with a C-C motif chemokine receptor, such as CCR2, CCR4, CCR6, CCR7, and CCR8, is not to be confused with the abbreviation CCR used herein for costimulatory chimeric receptors. For example, the term “CCR2” refers herein to C-C motif chemokine receptor 2, the term “CCR4” refers herein to C-C motif chemokine receptor 4, the term “CCR6” refers herein to C-C motif chemokine receptor 6, the term “CCR7” refers herein to C-C (or CC) motif chemokine receptor 7, and the term “CCR8” refers herein to C-C motif chemokine receptor 8. In some embodiments, a population of TILs, MILs, or PBLs is genetically modified to express a full-length chemokine receptor in order to induce a chemotactic response and Ca²⁺ flux on the population of TILs, MILs, or PBLs when encountering the ligand gradient to improve tumor tissue trafficking. In some embodiments, a population of TILs, MILs, or PBLs is genetically modified to express a C-X-3-C (or CX3C) motif chemokine receptor. In some embodiments, a population of TILs, MILs, or PBLs is genetically modified to express a X-C (or XC) motif chemokine receptor. The role of chemokine receptors in T-cell homing is described in Sackstein, et al., Lab. Invest. 2017, 97, 669-97, the disclosures of which are incorporated by reference herein.

In some embodiments, the chemokine receptor is a C-X-C motif chemokine receptor. In some embodiments, the chemokine receptor is a C-C motif chemokine receptor. In some embodiments, the chemokine receptor is a C-X-3-C motif chemokine receptor. In some embodiments, the chemokine receptor is a X-C motif chemokine receptor. In some embodiments, the chemokine receptor is selected from the group consisting of CXCR1, CXCR2, CXCR3, CXCR4, CXCR5, CCR2, CCR4, CCR6, CCR7, CCR8, and combinations thereof. In some embodiments, the chemokine receptor is selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof.

In some embodiments, the chemokine receptor is selected from the chemokine receptors given in Table 64.

TABLE 64
Amino acid sequences of exemplary chemokine receptor domains.

| Identifier | Sequence (One-Letter Amino Acid Symbols) | |
|---|---|---|
| SEQ ID NO: 640 | MSNITDPQMW DFDDLNFTGM PPADEDYSPC MLETETLNKY VVIIAYALVF LLSLLGNSLV | 60 |
| CXCR1 domain | MLVILYSRVG RSVTDVYLLN LALADLLFAL TLPIWAASKV NGWIFGTFLC KVVSLLKEVN | 120 |
| | FYSGILLLAC ISVDRYLAIV HATRTLTQKR HLVKFVCLGC WGLSMNLSLP FFLFRQAYHP | 180 |
| | NNSSPVCYEV LGNDTAKWRM VLRILPHTFG FIVPLFVMLF CYGFTLRTLF KAHMGQKHRA | 240 |
| | MRVIFAVVLI FLLCWLPYNL VLLADTLMRT QVIQESCERR NNIGRALDAT EILGFLHSCL | 300 |
| | NPIIYAFIGQ NFRHGFLKIL AMHGLVSKEF LARHRVTSYT SSSVNVSSNL | 350 |
| SEQ ID NO: 628 | MEDFNMESDS FEDFWKGEDL SNYSYSSTLP PFLLDAAPCE PESLEINKYF VVIIYALVFL | 60 |
| CXCR2 variant 1 | LSLIGNSLVM LVILYSRVGR SVTDVYLLNL ALADLLFALT LPIWAASKVN GWIFGTFLCK | 120 |
| and 2 domain | VVSLLKEVNF YSGILLLACI SVDRYLAIVH ATRTLTQKRY LVKFICLSIW GLSLLLALPV | 180 |
| | LLFRRTVYSS NVSPACYEDM GNNTANWRML LRILPQSFGF IVPLLIMLFC YGFTLRTLFK | 240 |
| | AHMGQKHRAM RVIFAVVLIF LLCWLPYNLV LLADTLMRTQ VIQETCERRN HIDRALDATE | 300 |
| | ILGILHSCLN PLIYAFIGQK FRHGLLKILA IHGLISKDSL PKDSRPSFVG SSSGHTSTTL | 360 |
| SEQ ID NO: 629 | MVLEVSDHQV LNDAEVAALL ENFSSSYDYG ENESDSCCTS PPCPQDFSLN FDRAFLPALY | 60 |
| CXCR3 variant 1 | SLLFLLGLLG NGAVAAVLLS RRTALSSTDT FLLHLAVADT LLVLTLPLWA VDAAVQWVFG | 120 |
| domain | SGLCKVAGAL FNINFYAGAL LLACISFDRY LNIVHATQLY RRGPPARVTL TCLAVWGLCL | 180 |
| | LFALPDFIFL SAHHDERLNA THCQYNFPQV GRTALRVLQL VAGFLLPLLV MAYCYAHILA | 240 |
| | VLLVSRGQRR LRAMRLVVVV VVAFALCWTP YHLVVLVDIL MDLGALARNC GRESRVDVAK | 300 |
| | SVTSGLGYMH CCLNPLLYAF VGVKFRERMW MLLLRLGCPN QRGLQRQPSS SRRDSSWSET | 360 |
| | SEASYSGL | 368 |
| SEQ ID NO: 630 | MELRKYGPGR LAGTVIGGAA QSKSQTKSDS ITKEFLPGLY TAPSSPFPPS QVSDHQVIND | 60 |
| CXCR3 variant 2 | AEVAALLENF SSSYDYGENE SDSCCTSPPC PQDFSLNFDR AFLPALYSLL FLLGLLGNGA | 120 |
| domain | VAAVLLSRRT ALSSTDTFLL HLAVADTLLV LTLPLWAVDA AVQWVFGSGL CKVAGALFNI | 180 |
| | NFYAGALLLA CISFDRYLNI VHATQLYRRG PPARVTLTCL AVWGLCLLFA LPDFIFLSAH | 240 |
| | HDERLNATHC QYNFPQVGRT ALRVLQLVAG FLLPLLVMAY CYAHILAVLL VSRGQRRIRA | 300 |
| | MRLVVVVVVA FALCWTPYHL VVLVDILMDL GALARNCGRE SRVDVAKSVT SGLGYMHCCL | 360 |
| | NPLLYAFVGV KFRERMWMLL LRLGCPNQRG LQRQPSSSRR DSSWSETSEA SYSGL | 415 |
| SEQ ID NO: 631 | MSIPLPLLQI YTSDNYTEEM GSGDYDSMKE PCFREENANF NKIFLPTIYS IIFLTGIVGN | 60 |
| CXCR4 variant 1 | GLVILVMGYQ KKLRSMTDKY RLHLSVADLL FVITLPFWAV DAVANWYFGN FLCKAVHVIY | 120 |
| domain | TVNLYSSVLI LAFISLDRYL AIVHATNSQR PRKLLAEKVV YVGVWIPALL LTIPDFIFAN | 180 |
| | VSEADDRYIC DRFYPNDLWV VVFQFQHIMV GLILPGIVIL SCYCIIISKL SHSKGHQKRK | 240 |
| | ALKTTVILIL AFFACWLPYY IGISIDSFIL LEIIKQGCEF ENTVHKWISI TEALAFFHCC | 300 |
| | LNPILYAFLG AKFKTSAQHA LTSVSRGSSL KILSKGKRGG HSSVSTESES SSFHSS | 356 |
| SEQ ID NO: 632 | MEGISIYTSD NYTEEMGSGD YDSMKEPCFR EENANFNKIF LPTIYSIIFL TGIVGNGLVI | 60 |
| CXCR4 variant 2 | LVMGYQKKLR SMTDKYRLHL SVADLLFVIT LPFWAVDAVA NWYFGNFLCK AVHVIYTVNL | 120 |
| domain | YSSVLILAFI SLDRYLAIVH ATNSQRPRKL LAEKVVYVGV WIPALLLTIP DFIFANVSEA | 180 |
| | DDRYICDRFY PNDLWVVVFQ FQHIMVGLIL PGIVILSCYC IIISKLSHSK GHQKRKALKT | 240 |
| | TVILILAFFA CWLPYYIGIS IDSFILLEII KQGCEFENTV HKWISITEAL AFFHCCINPI | 300 |
| | LYAFLGAKFK TSAQHALTSV SRGSSLKILS KGKRGGHSSV STESESSSFH SS | 352 |
| SEQ ID NO: 633 | MEGISENAPL PNVPNAPSDK HEDGKRPTHR RSARLGEEVP FVHFLTLPPN IPQAPKGLRF | 60 |
| CXCR4 variant 3 | KTAFSLPTTS CLKPRMIYTS DNYTEEMGSG DYDSMKEPCF REENANFNKI FLPTIYSIIF | 120 |
| domain | LTGIVGNGLV ILVMGYQKKL RSMTDKYRLH LSVADLLFVI TLPFWAVDAV ANWYFGNFLC | 180 |
| | KAVHVIYTVN LYSSVLILAF ISLDRYLAIV HATNSQRPRK LLAEKVVYVG VWIPALLLTI | 240 |
| | PDFIFANVSE ADDRYICDRF YPNDLWVVVF QFQHIMVGLI LPGIVILSCY CIIISKLSHS | 300 |
| | KGHQKRKALK TTVILILAFF ACWLPYYIGI SIDSFILLEI IKQGCEFENT VHKWISITEA | 360 |
| | LAFFHCCLNP ILYAFLGAKF KTSAQHALTS VSRGSSLKIL SKGKRGGHSS VSTESESSSF | 420 |
| | SSH | 423 |
| SEQ ID NO: 634 | MEGISENAPL PNVPNAPSDK HEDGKRPTHR RSARLGEEIY TSDNYTEEMG SGDYDSMKEP | 60 |
| CXCR4 variant 4 | CFREENANFN KIFLPTIYSI IFLTGIVGNG LVILVMGYQK KLRSMTDKYR LHLSVADLLF | 120 |
| domain | VITLPFWAVD AVANWYFGNF LCKAVHVIYT VNLYSSVLIL AFISLDRYLA IVHATNSQRP | 180 |
| | RKLLAEKVVY VGVWIPALLL TIPDFIFANV SEADDRYICD RFYPNDLWVV VFQFQHIMVG | 240 |
| | LILPGIVILS CYCIIISKLS HSKGHQKRKA LKTTVILILA FFACWLPYYI GISIDSFILL | 300 |
| | EIIKQGCEFE NTVHKWISIT EALAFFHCCL NPILYAFLGA KFKTSAQHAL TSVSRGSSLK | 360 |
| | ILSKGKRGGH SSVSTESESS SFHSS | 385 |
| SEQ ID NO: 635 | MGSGDYDSMK EPCFREENAN FNKIFLPTIY SIIFLTGIVG NGLVILVMGY QKKLRSMTDK | 60 |
| CXCR4 variant 5 | YRLHLSVADL LFVITLPFWA VDAVANWYFG NFLCKAVHVI YTVNLYSSVL ILAFISLDRY | 120 |
| domain | LAIVHATNSQ RPRKLLAEKV VYVGVWIPAL LITIPDFIFA NVSEADDRYI CDRFYPNDLW | 180 |
| | VVVFQFQHIM VGLILPGIVI LSCYCIIISK LSHSKGHQKR KALKTTVILI LAFFACWLPY | 240 |
| | YIGISIDSFI LLEIIKQGCE FENTVHKWIS ITEALAFFHC CLNPILYAFL GAKFKTSAQH | 300 |
| | ALTSVSRGSS LKILSKGKRG GHSSVSTESE SSSFHSS | 337 |
| SEQ ID NO: 636 | MNYPLTLEMD LENLEDLFWE LDRLDNYNDT SLVENHLCPA TEGPLMASFK AVFVPVAYSL | 60 |
| CXCR5 variant 1 | IFLIGVIGNV LVLVILERHR QTRSSTETFL FHLAVADLLL VFILPFAVAE GSVGWVLGTF | 120 |
| domain | LCKTVIALHK VNFYCSSLLL ACIAVDRYLA IVHAVHAYRH RRLLSIHITC GTIWLVGFLL | 180 |
| | ALPEILFAKV SQGHHNNSLP RCTFSQENQA ETHAWFTSRF LYHVAGFLLP MLVMGWCYVG | 240 |
| | VVHRLRQAQR RPQRQKAVRV AILVTSIFFL CWSPYHIVIF LDTLARLKAV DNTCKINGSL | 300 |
| | PVAITMCEFL GLAHCCLNPM LYTFAGVKFR SDLSRLLTKL GCTGPASLCQ LFPSWRRSSL | 360 |
| | SESENATSLT TF | 372 |
| SEQ ID NO: 637 | MASFKAVFVP VAYSLIFLLG VIGNVLVLVI LERHRQTRSS TETFLFHLAV ADLLLVFILP | 60 |
| CXCR5 variant 2 | FAVAEGSVGW VLGTFLCKTV IALHKVNFYC SSLLLACIAV DRYLAIVHAV HAYRHRRLLS | 120 |
| domain | IHITCGTIWL VGFLLALPEI LFAKVSQGHH NNSLPRCTFS QENQAETHAW FTSRFLYHVA | 180 |
| | GFLLPMLVMG WCYVGVVHRL RQAQRRPQRQ KAVRVAILVT SIFFLCWSPY HIVIFLDTLA | 240 |
| | RLKAVDNTCK LNGSLPVAIT MCEFLGLAHC CLNPMLYTFA GVKFRSDLSR LLTKLGCTGP | 300 |
| | ASLCQLFPSW RRSSLSESEN ATSLTTF | 327 |
| SEQ ID NO: 638 | MLSTSRSRFI RNTNESGEEV TTFFDYDYGA PCHKFDVKQI GAQLLPPLYS LVFIFGFVGN | 60 |
| CCR2 variant A | MLVVLILINC KKLKCLTDIY LLNLAISDLL FLITLPLWAH SAANEWVFGN AMCKLFTGLY | 120 |
| domain | HIGYFGGIFF IILLTIDRYL AIVHAVFALK ARTVTFGVVT SVITWLVAVF ASVPGIIFTK | 180 |
| | CQKEDSVYVC GPYFPRGWNN FHTIMRNILG LVLPLLIMVI CYSGILKTLL RCRNEKKRHR | 240 |
| | AVRVIFTIMI VYFLFWTPYN IVILLNTFQE FFGLSNCEST SQLDQATQVT ETLGMTHCCI | 300 |
| | NPIIYAFVGE KFRSLFHIAL GCRIAPLQKP VCGGPGVRPG KNVKVTTQGL LDGRGKGKSI | 360 |
| | GRAPEASLQD KEGA | 374 |
| SEQ ID NO: 639 | MLSTSRSRFI RNTNESGEEV TTFFDYDYGA PCHKFDVKQI GAQLLPPLYS LVFIFGFVGN | 60 |
| CCR2 variant B | MLVVLILINC KKLKCLTDIY LLNLAISDLL FLITLPLWAH SAANEWVFGN AMCKLFTGLY | 120 |
| domain | HIGYFGGIFF IILLTIDRYL AIVHAVFALK ARTVTFGVVT SVITWLVAVF ASVPGIIFTK | 180 |
| | CQKEDSVYVC GPYFPRGWNN FHTIMRNILG LVLPLLIMVI CYSGILKTLL RCRNEKKRHR | 240 |
| | AVRVIFTIMI VYFLFWTPYN IVILLNTFQE FFGLSNCEST SQLDQATQVT ETLGMTHCCI | 300 |
| | NPIIYAFVGE KFRRYLSVFF RKHITKRFCK QCPVFYRETV DGVTSTNTPS TGEQEVSAGL | 360 |
| SEQ ID NO: 640 | MNPTDIADTT LDESIYSNYY LYESIPKPCT KEGIKAFGEL FLPPLYSLVF VFGLLGNSVV | 60 |
| CCR4 domain | VLVLFKYKRL RSMTDVYLLN LAISDLLFVF SLPFWGYYAA DQWVFGLGLC KMISWMYLVG | 120 |
| | FYSGIFFVML MSIDRYLAIV HAVFSLRART LTYGVITSLA TWSVAVFASL PGFLFSTCYT | 180 |
| | ERNHTYCKTK YSLNSTTWKV LSSLEINILG LVIPLGIMLF CYSMIIRTLQ HCKNEKKNKA | 240 |
| | VKMIFAVVVL FLGFWTPYNI VLFLETLVEL EVLQDCTFER YLDYAIQATE TLAFVHCCLN | 300 |
| | PIIYFFLGEK FRKYILQLFK TCRGLFVLCQ YCGLLQIYSA DTPSSSYTQS TMDHDLHDAL | 360 |
| SEQ ID NO: 641 | MSGESMNFSD VFDSSEDYFV SVNTSYYSVD SEMLLCSLQE VRQFSRLFVP IAYSLICVFG | 60 |
| CCR6 variant 1 | LLGNILVVIT FAFYKKARSM TDVYLLNMAI ADILFVLTLP FWAVSHATGA WVFSNATCKL | 120 |
| and 2 domain | LKGIYAINFN CGMLLLTCIS MDRYIAIVQA TKSFRLRSRT LPRSKIICLV VWGLSVIISS | 180 |
| | STFVFNQKYN TQGSDVCEPK YQTVSEPIRW KLLMLGLELL FGFFIPLMFM IFCYTFIVKT | 240 |
| | LVQAQNSKRH KAIRVIIAVV LVFLACQIPH NMVLLVTAAN LGKMNRSCQS EKLIGYTKTV | 300 |
| | TEVLAFLHCC LNPVLYAFIG QKFRNYFLKI LKDLWCVRRK YKSSGFSCAG RYSENISRQT | 360 |
| | SETADNDNAS SFTM | 374 |
| SEQ ID NO: 642 | MDLGKPMKSV LVVALLVIFQ VCLCQDEVTD DYIGDNTTVD YTLFESLCSK KDVRNFKAWF | 60 |
| CCR7 variant 1 | LPIMYSIICF VGLLGNGLVV LTYIYFKRLK TMTDTYLLNL AVADILFLLT LPFWAYSAAK | 120 |
| domain | SWVFGVHFCK LIFAIYKMSF FSGMLLLLCI SIDRYVAIVQ AVSAHRHRAR VLLISKLSCV | 180 |
| | GIWILATVLS IPELLYSDLQ RSSSEQAMRC SLITEHVEAF ITIQVAQMVI GFLVPLLAMS | 240 |
| | FCYLVIIRTL LQARNFERNK AIKVIIAVVV VFIVFQLPYN GVVLAQTVAN FNITSSTCEL | 300 |
| | SKQLNIAYDV TYSLACVRCC VNPFLYAFIG VKFRNDLFKL FKDLGCLSQE QLRQWSSCRH | 360 |
| | IRRSSMSVEA ETTTTFSP | 378 |
| SEQ ID NO: 643 | MYSIICFVGL LGNGLVVLTY IYFKRLKTMT DTYLLNLAVA DILFLLTLPF WAYSAAKSWV | 60 |
| CCR7 variant 2 | FGVHFCKLIF AIYKMSFFSG MLLLLCISID RYVAIVQAVS AHRHRARVLL ISKLSCVGIW | 120 |
| domain | ILATVLSIPE LLYSDLQRSS SEQAMRCSLI TEHVEAFITI QVAQMVIGFL VPLLAMSFCY | 180 |
| | LVIIRTLLQA RNFERNKAIK VIIAVVVVFI VFQLPYNGVV LAQTVANFNI TSSTCELSKQ | 240 |
| | LNIAYDVTYS LACVRCCVNP FLYAFIGVKF RNDLFKLFKD LGCLSQEQLR QWSSCRHIRR | 300 |
| | SSMSVEAETT TTFSP | 315 |
| SEQ ID NO: 644 | MKSVLVVALL VIFQVCLCQD EVTDDYIGDN TTVDYTLFES LCSKKDVRNF KAWFLPIMYS | 60 |
| CCR7 variant 3, | IICFVGLLGN GLVVLTYIYF KRLKTMTDTY LINLAVADIL FLLTLPFWAY SAAKSWVFGV | 120 |
| 4, and 5 domain | HFCKLIFAIY KMSFFSGMLL LLCISIDRYV AIVQAVSAHR HRARVLLISK LSCVGIWILA | 180 |
| | TVLSIPELLY SDLQRSSSEQ AMRCSLITEH VEAFITIQVA QMVIGFLVPL LAMSFCYLVI | 240 |
| | IRTILQARNF ERNKAIKVII AVVVVFIVFQ LPYNGVVLAQ TVANFNITSS TCELSKQLNI | 300 |
| | AYDVTYSLAC VRCCVNPFLY AFIGVKFRND LFKLFKDLGC LSQEQLRQWS SCRHIRRSSM | 360 |
| | SVEAETTTTF SP | 372 |
| SEQ ID NO: 645 | MDYTLDLSVT TVTDYYYPDI FSSPCDAELI QTNGKLLLAV FYCLLFVFSL LGNSLVILVL | 60 |
| CCR8 domain | VVCKKLRSIT DVYLLNLALS DLLFVFSFPF QTYYLLDQWV FGTVMCKVVS GFYYIGFYSS | 120 |
| | MFFITLMSVD RYLAVVHAVY ALKVRTIRMG TTLCLAVWLT AIMATIPLLV FYQVASEDGV | 180 |
| | LQCYSFYNQQ TLKWKIFTNF KMNILGLLIP FTIFMFCYIK ILHQLKRCQN HNKTKAIRLV | 240 |
| | LIVVIASLLF WVPFNVVLFL TSLHSMHILD GCSISQQLTY ATHVTEIISF THCCVNPVIY | 300 |
| | AFVGEKFKKH LSEIFQKSCS QIFNYLGRQM PRESCEKSSS CQQHSSRSSS VDYIL | 355 |

In an embodiment, a chemokine receptor of the present invention includes a domain comprising the amino acid sequence of SEQ ID NO:627, or conservative amino acid substitutions thereof, or fragments, variants, or derivatives thereof, or an amino acid sequence that is at least 99% identical to the sequence given in SEQ ID NO:627, at least 98% identical to the sequence given in SEQ ID NO:627, at least 97% identical to the sequence given in SEQ ID NO:627, at least 96% identical to the sequence given in SEQ ID NO:627, at least 95% identical to the sequence given in SEQ ID NO:627, at least 90% identical to the sequence given in SEQ ID NO:627, at least 85% identical to the sequence given in SEQ ID NO:627, or at least 80% identical to the sequence given in SEQ ID NO:627. In an embodiment, a chemokine receptor of the present invention includes a nucleotide domain that encodes the amino acid sequence of SEQ ID NO:627, or conservative amino acid substitutions thereof, or fragments, variants, or derivatives thereof.

In an embodiment, a chemokine receptor of the present invention includes a domain comprising the amino acid sequence of SEQ ID NO:628, or conservative amino acid substitutions thereof, or fragments, variants, or derivatives thereof, or an amino acid sequence that is at least 99% identical to the sequence given in SEQ ID NO:628, at least 98% identical to the sequence given in SEQ ID NO:628, at least 97% identical to the sequence given in SEQ ID NO:628, at least 96% identical to the sequence given in SEQ ID NO:628, at least 95% identical to the sequence given in SEQ ID NO:628, at least 90% identical to the sequence given in SEQ ID NO:628, at least 85% identical to the sequence given in SEQ ID NO:628, or at least 80% identical to the sequence given in SEQ ID NO:628. In an embodiment, a chemokine receptor of the present invention includes a nucleotide domain that encodes the amino acid sequence of SEQ ID NO:628, or conservative amino acid substitutions thereof, or fragments, variants, or derivatives thereof.

In an embodiment, a chemokine receptor of the present invention includes a domain comprising the amino acid sequence of SEQ ID NO:629, or conservative amino acid substitutions thereof, or fragments, variants, or derivatives thereof, or an amino acid sequence that is at least 99% identical to the sequence given in SEQ ID NO:629, at least 98% identical to the sequence given in SEQ ID NO:629, at least 97% identical to the sequence given in SEQ ID NO:629, at least 96% identical to the sequence given in SEQ ID NO:629, at least 95% identical to the sequence given in SEQ ID NO:629, at least 90% identical to the sequence given in SEQ ID NO:629, at least 85% identical to the sequence given in SEQ ID NO:629, or at least 80% identical to the sequence given in SEQ ID NO:629. In an embodiment, a chemokine receptor of the present invention includes a nucleotide domain that encodes the amino acid sequence of SEQ ID NO:629, or conservative amino acid substitutions thereof, or fragments, variants, or derivatives thereof.

In an embodiment, a chemokine receptor of the present invention includes a domain comprising the amino acid sequence of SEQ ID NO:630, or conservative amino acid substitutions thereof, or fragments, variants, or derivatives thereof, or an amino acid sequence that is at least 99% identical to the sequence given in SEQ ID NO:630, at least 98% identical to the sequence given in SEQ ID NO:630, at least 97% identical to the sequence given in SEQ ID NO:630, at least 96% identical to the sequence given in SEQ ID NO:630, at least 95% identical to the sequence given in SEQ ID NO:630, at least 90% identical to the sequence given in SEQ ID NO:630, at least 85% identical to the sequence given in SEQ ID NO:630, or at least 80% identical to the sequence given in SEQ ID NO:630. In an embodiment, a chemokine receptor of the present invention includes a nucleotide domain that encodes the amino acid sequence of SEQ ID NO:630, or conservative amino acid substitutions thereof, or fragments, variants, or derivatives thereof.

In an embodiment, a chemokine receptor of the present invention includes a domain comprising the amino acid sequence of SEQ ID NO:631, or conservative amino acid substitutions thereof, or fragments, variants, or derivatives thereof, or an amino acid sequence that is at least 99% identical to the sequence given in SEQ ID NO:631, at least 98% identical to the sequence given in SEQ ID NO:631, at least 97% identical to the sequence given in SEQ ID NO:631, at least 96% identical to the sequence given in SEQ ID NO:631, at least 95% identical to the sequence given in SEQ ID NO:631, at least 90% identical to the sequence given in SEQ ID NO:631, at least 85% identical to the sequence given in SEQ ID NO:631, or at least 80% identical to the sequence given in SEQ ID NO:631. In an embodiment, a chemokine receptor of the present invention includes a nucleotide domain that encodes the amino acid sequence of SEQ ID NO:631, or conservative amino acid substitutions thereof, or fragments, variants, or derivatives thereof.

In an embodiment, a chemokine receptor of the present invention includes a domain comprising the amino acid sequence of SEQ ID NO:632, or conservative amino acid substitutions thereof, or fragments, variants, or derivatives thereof, or an amino acid sequence that is at least 99% identical to the sequence given in SEQ ID NO:632, at least 98% identical to the sequence given in SEQ ID NO:632, at least 97% identical to the sequence given in SEQ ID NO:632, at least 96% identical to the sequence given in SEQ ID NO:632, at least 95% identical to the sequence given in SEQ ID NO:632, at least 90% identical to the sequence given in SEQ ID NO:632, at least 85% identical to the sequence given in SEQ ID NO:632, or at least 80% identical to the sequence given in SEQ ID NO:632. In an embodiment, a chemokine receptor of the present invention includes a nucleotide domain that encodes the amino acid sequence of SEQ ID NO:632, or conservative amino acid substitutions thereof, or fragments, variants, or derivatives thereof.

In an embodiment, a chemokine receptor of the present invention includes a domain comprising the amino acid sequence of SEQ ID NO:633, or conservative amino acid substitutions thereof, or fragments, variants, or derivatives thereof, or an amino acid sequence that is at least 99% identical to the sequence given in SEQ ID NO:633, at least 98% identical to the sequence given in SEQ ID NO:633, at least 97% identical to the sequence given in SEQ ID NO:633, at least 96% identical to the sequence given in SEQ ID NO:633, at least 95% identical to the sequence given in SEQ ID NO:633, at least 90% identical to the sequence given in SEQ ID NO:633, at least 85% identical to the sequence given in SEQ ID NO:633, or at least 80% identical to the sequence given in SEQ ID NO:633. In an embodiment, a chemokine receptor of the present invention includes a nucleotide domain that encodes the amino acid sequence of SEQ ID NO:633, or conservative amino acid substitutions thereof, or fragments, variants, or derivatives thereof.

In an embodiment, a chemokine receptor of the present invention includes a domain comprising the amino acid sequence of SEQ ID NO:634, or conservative amino acid substitutions thereof, or fragments, variants, or derivatives thereof, or an amino acid sequence that is at least 99% identical to the sequence given in SEQ ID NO:634, at least 98% identical to the sequence given in SEQ ID NO:634, at least 97% identical to the sequence given in SEQ ID NO:634, at least 96% identical to the sequence given in SEQ ID NO:634, at least 95% identical to the sequence given in SEQ ID NO:634, at least 90% identical to the sequence given in SEQ ID NO:634, at least 85% identical to the sequence given in SEQ ID NO:634, or at least 80% identical to the sequence given in SEQ ID NO:634. In an embodiment, a chemokine receptor of the present invention includes a nucleotide domain that encodes the amino acid sequence of SEQ ID NO:634, or conservative amino acid substitutions thereof, or fragments, variants, or derivatives thereof.

In an embodiment, a chemokine receptor of the present invention includes a domain comprising the amino acid sequence of SEQ ID NO:635, or conservative amino acid substitutions thereof, or fragments, variants, or derivatives thereof, or an amino acid sequence that is at least 99% identical to the sequence given in SEQ ID NO:635, at least 98% identical to the sequence given in SEQ ID NO:635, at least 97% identical to the sequence given in SEQ ID NO:635, at least 96% identical to the sequence given in SEQ ID NO:635, at least 95% identical to the sequence given in SEQ ID NO:635, at least 90% identical to the sequence given in SEQ ID NO:635, at least 85% identical to the sequence given in SEQ ID NO:635, or at least 80% identical to the sequence given in SEQ ID NO:635. In an embodiment, a chemokine receptor of the present invention includes a nucleotide domain that encodes the amino acid sequence of SEQ ID NO:635, or conservative amino acid substitutions thereof, or fragments, variants, or derivatives thereof.

In an embodiment, a chemokine receptor of the present invention includes a domain comprising the amino acid sequence of SEQ ID NO:636, or conservative amino acid substitutions thereof, or fragments, variants, or derivatives thereof, or an amino acid sequence that is at least 99% identical to the sequence given in SEQ ID NO:636, at least 98% identical to the sequence given in SEQ ID NO:636, at least 97% identical to the sequence given in SEQ ID NO:636, at least 96% identical to the sequence given in SEQ ID NO:636, at least 95% identical to the sequence given in SEQ ID NO:636, at least 90% identical to the sequence given in SEQ ID NO:636, at least 85% identical to the sequence given in SEQ ID NO:636, or at least 80% identical to the sequence given in SEQ ID NO:636. In an embodiment, a chemokine receptor of the present invention includes a nucleotide domain that encodes the amino acid sequence of SEQ ID NO:636, or conservative amino acid substitutions thereof, or fragments, variants, or derivatives thereof.

In an embodiment, a chemokine receptor of the present invention includes a domain comprising the amino acid sequence of SEQ ID NO:637, or conservative amino acid substitutions thereof, or fragments, variants, or derivatives thereof, or an amino acid sequence that is at least 99% identical to the sequence given in SEQ ID NO:637, at least 98% identical to the sequence given in SEQ ID NO:637, at least 97% identical to the sequence given in SEQ ID NO:637, at least 96% identical to the sequence given in SEQ ID NO:637, at least 95% identical to the sequence given in SEQ ID NO:637, at least 90% identical to the sequence given in SEQ ID NO:637, at least 85% identical to the sequence given in SEQ ID NO:637, or at least 80% identical to the sequence given in SEQ ID NO:637. In an embodiment, a chemokine receptor of the present invention includes a nucleotide domain that encodes the amino acid sequence of SEQ ID NO:637, or conservative amino acid substitutions thereof, or fragments, variants, or derivatives thereof.

In an embodiment, a chemokine receptor of the present invention includes a domain comprising the amino acid sequence of SEQ ID NO:638, or conservative amino acid substitutions thereof, or fragments, variants, or derivatives thereof, or an amino acid sequence that is at least 99% identical to the sequence given in SEQ ID NO:638, at least 98% identical to the sequence given in SEQ ID NO:638, at least 97% identical to the sequence given in SEQ ID NO:638, at least 96% identical to the sequence given in SEQ ID NO:638, at least 95% identical to the sequence given in SEQ ID NO:638, at least 90% identical to the sequence given in SEQ ID NO:638, at least 85% identical to the sequence given in SEQ ID NO:638, or at least 80% identical to the sequence given in SEQ ID NO:638. In an embodiment, a chemokine receptor of the present invention includes a nucleotide domain that encodes the amino acid sequence of SEQ ID NO:638, or conservative amino acid substitutions thereof, or fragments, variants, or derivatives thereof.

In an embodiment, a chemokine receptor of the present invention includes a domain comprising the amino acid sequence of SEQ ID NO:639, or conservative amino acid substitutions thereof, or fragments, variants, or derivatives thereof, or an amino acid sequence that is at least 99% identical to the sequence given in SEQ ID NO:639, at least 98% identical to the sequence given in SEQ ID NO:639, at least 97% identical to the sequence given in SEQ ID NO:639, at least 96% identical to the sequence given in SEQ ID NO:639, at least 95% identical to the sequence given in SEQ ID NO:639, at least 90% identical to the sequence given in SEQ ID NO:639, at least 85% identical to the sequence given in SEQ ID NO:639, or at least 80% identical to the sequence given in SEQ ID NO:639. In an embodiment, a chemokine receptor of the present invention includes a nucleotide domain that encodes the amino acid sequence of SEQ ID NO:639, or conservative amino acid substitutions thereof, or fragments, variants, or derivatives thereof.

In an embodiment, a chemokine receptor of the present invention includes a domain comprising the amino acid sequence of SEQ ID NO:640, or conservative amino acid substitutions thereof, or fragments, variants, or derivatives thereof, or an amino acid sequence that is at least 99% identical to the sequence given in SEQ ID NO:640, at least 98% identical to the sequence given in SEQ ID NO:640, at least 97% identical to the sequence given in SEQ ID NO:640, at least 96% identical to the sequence given in SEQ ID NO:640, at least 95% identical to the sequence given in SEQ ID NO:640, at least 90% identical to the sequence given in SEQ ID NO:640, at least 85% identical to the sequence given in SEQ ID NO:640, or at least 80% identical to the sequence given in SEQ ID NO:640. In an embodiment, a chemokine receptor of the present invention includes a nucleotide domain that encodes the amino acid sequence of SEQ ID NO:640, or conservative amino acid substitutions thereof, or fragments, variants, or derivatives thereof.

In an embodiment, a chemokine receptor of the present invention includes a domain comprising the amino acid sequence of SEQ ID NO:641, or conservative amino acid substitutions thereof, or fragments, variants, or derivatives thereof, or an amino acid sequence that is at least 99% identical to the sequence given in SEQ ID NO:641, at least 98% identical to the sequence given in SEQ ID NO:641, at least 97% identical to the sequence given in SEQ ID NO:641, at least 96% identical to the sequence given in SEQ ID NO:641, at least 95% identical to the sequence given in SEQ ID NO:641, at least 90% identical to the sequence given in SEQ ID NO:641, at least 85% identical to the sequence given in SEQ ID NO:641, or at least 80% identical to the sequence given in SEQ ID NO:641. In an embodiment, a chemokine receptor of the present invention includes a nucleotide domain that encodes the amino acid sequence of SEQ ID NO:641, or conservative amino acid substitutions thereof, or fragments, variants, or derivatives thereof.

In an embodiment, a chemokine receptor of the present invention includes a domain comprising the amino acid sequence of SEQ ID NO:642, or conservative amino acid substitutions thereof, or fragments, variants, or derivatives thereof, or an amino acid sequence that is at least 99% identical to the sequence given in SEQ ID NO:642, at least 98% identical to the sequence given in SEQ ID NO:642, at least 97% identical to the sequence given in SEQ ID NO:642, at least 96% identical to the sequence given in SEQ ID NO:642, at least 95% identical to the sequence given in SEQ ID NO:642, at least 90% identical to the sequence given in SEQ ID NO:642, at least 85% identical to the sequence given in SEQ ID NO:642, or at least 80% identical to the sequence given in SEQ ID NO:642. In an embodiment, a chemokine receptor of the present invention includes a nucleotide domain that encodes the amino acid sequence of SEQ ID NO:642, or conservative amino acid substitutions thereof, or fragments, variants, or derivatives thereof.

In an embodiment, a chemokine receptor of the present invention includes a domain comprising the amino acid sequence of SEQ ID NO:643, or conservative amino acid substitutions thereof, or fragments, variants, or derivatives thereof, or an amino acid sequence that is at least 99% identical to the sequence given in SEQ ID NO:643, at least 98% identical to the sequence given in SEQ ID NO:643, at least 97% identical to the sequence given in SEQ ID NO:643, at least 96% identical to the sequence given in SEQ ID NO:643, at least 95% identical to the sequence given in SEQ ID NO:643, at least 90% identical to the sequence given in SEQ ID NO:643, at least 85% identical to the sequence given in SEQ ID NO:643, or at least 80% identical to the sequence given in SEQ ID NO:643. In an embodiment, a chemokine receptor of the present invention includes a nucleotide domain that encodes the amino acid sequence of SEQ ID NO:643, or conservative amino acid substitutions thereof, or fragments, variants, or derivatives thereof.

In an embodiment, a chemokine receptor of the present invention includes a domain comprising the amino acid sequence of SEQ ID NO:644, or conservative amino acid substitutions thereof, or fragments, variants, or derivatives thereof, or an amino acid sequence that is at least 99% identical to the sequence given in SEQ ID NO:644, at least 98% identical to the sequence given in SEQ ID NO:644, at least 97% identical to the sequence given in SEQ ID NO:644, at least 96% identical to the sequence given in SEQ ID NO:644, at least 95% identical to the sequence given in SEQ ID NO:644, at least 90% identical to the sequence given in SEQ ID NO:644, at least 85% identical to the sequence given in SEQ ID NO:644, or at least 80% identical to the sequence given in SEQ ID NO:644. In an embodiment, a chemokine receptor of the present invention includes a nucleotide domain that encodes the amino acid sequence of SEQ ID NO:644, or conservative amino acid substitutions thereof, or fragments, variants, or derivatives thereof.

In an embodiment, a chemokine receptor of the present invention includes a domain comprising the amino acid sequence of SEQ ID NO:645, or conservative amino acid substitutions thereof, or fragments, variants, or derivatives thereof, or an amino acid sequence that is at least 99% identical to the sequence given in SEQ ID NO:645, at least 98% identical to the sequence given in SEQ ID NO:645, at least 97% identical to the sequence given in SEQ ID NO:645, at least 96% identical to the sequence given in SEQ ID NO:645, at least 95% identical to the sequence given in SEQ ID NO:645, at least 90% identical to the sequence given in SEQ ID NO:645, at least 85% identical to the sequence given in SEQ ID NO:645, or at least 80% identical to the sequence given in SEQ ID NO:645. In an embodiment, a chemokine receptor of the present invention includes a nucleotide domain that encodes the amino acid sequence of SEQ ID NO:645, or conservative amino acid substitutions thereof, or fragments, variants, or derivatives thereof.

In some embodiments, the chemokine receptor is a protein encoded by a nucleotide encoding a C-X-C motif chemokine receptor. In some embodiments, the chemokine receptor is a protein encoded by a nucleotide encoding a C-C motif chemokine receptor. In some embodiments, the chemokine receptor is a protein encoded by a nucleotide encoding a C-X-3-C motif chemokine receptor. In some embodiments, the chemokine receptor is a protein encoded by a nucleotide encoding a X-C motif chemokine receptor. In some embodiments, the chemokine receptor is selected from the group consisting of CXCR1, CXCR2, CXCR3, CXCR4, CXCR5, CCR2, CCR4, CCR6, CCR7, CCR8, and combinations thereof. In some embodiments, the chemokine receptor is selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof.

In some embodiments, the chemokine receptor is a protein encoded by the nucleotides given in Table 65.

TABLE 65
Exemplary nucleotide sequences encoding exemplary chemokine receptor
domains.

| Identifier | Sequence (One-Letter Nucleotide Symbols) | |
|---|---|---|
| SEQ ID NO: 646 | ACTCTGATCT CTGACTGCAG CTCCTACTGT TGGACACACC TGGCCGGTGC TTCAGTTAGA | 60 |
| CXCR1 domain | TCAAACCATT GCTGAAACTG AAGAGGACAT GTCAAATATT ACAGATCCAC AGATGTGGGA | 120 |
| | TTTTGATGAT CTAAATTTCA CTGGCATGCC ACCTGCAGAT GAAGATTACA GCCCCTGTAT | 180 |
| | GCTAGAAACT GAGACACTCA ACAAGTATGT TGTGATCATC GCCTATGCCC TAGTGTTCCT | 240 |
| | GCTGAGCCTG CTGGGAAACT CCCTGGTGAT GCTGGTCATC TTATACAGCA GGGTCGGCCG | 300 |
| | CTCCGTCACT GATGTCTACC TGCTGAACCT GGCCTTGGCC GACCTACTCT TTGCCCTGAC | 360 |
| | CTTGCCCATC TGGGCCGCCT CCAAGGTGAA TGGCTGGATT TTTGGCACAT TCCTGTGCAA | 420 |
| | GGTGGTCTCA CTCCTGAAGG AAGTCAACTT CTACAGTGGC ATCCTGCTGT TGGCCTGCAT | 480 |
| | CAGTGTGGAC CGTTACCTGG CCATTGTCCA TGCCACACGC ACACTGACCC AGAAGCGTCA | 540 |
| | CTTGGTCAAG TTTGTTTGTC TTGGCTGCTG GGGACTGTCT ATGAATCTGT CCCTGCCCTT | 600 |
| | CTTCCTTTTC CGCCAGGCTT ACCATCCAAA CAATTCCAGT CCAGTTTGCT ATGAGGTCCT | 660 |
| | GGGAAATGAC ACAGCAAAAT GGCGGATGGT GTTGCGGATC CTGCCTCACA CCTTTGGCTT | 720 |
| | CATCGTGCCG CTGTTTGTCA TGCTGTTCTG CTATGGATTC ACCCTGCGTA CACTGTTTAA | 780 |
| | GGCCCACATG GGGCAGAAGC ACCGAGCCAT GAGGGTCATC TTTGCTGTCG TCCTCATCTT | 840 |
| | CCTGCTTTGC TGGCTGCCCT ACAACCTGGT CCTGCTGGCA GACACCCTCA TGAGGACCCA | 900 |
| | GGTGATCCAG GAGAGCTGTG AGCGCCGCAA CAACATCGGC CGGGCCCTGG ATGCCACTGA | 960 |
| | GATTCTGGGA TTTCTCCATA GCTGCCTCAA CCCCATCATC TACGCCTTCA TCGGCCAAAA | 1020 |
| | TTTTCGCCAT GGATTCCTCA AGATCCTGGC TATGCATGGC CTGGTCAGCA AGGAGTTCTT | 1080 |
| | GGCACGTCAT CGTGTTACCT CCTACACTTC TTCGTCTGTC AATGTCTCTT CCAACCTCTG | 1140 |
| | AAAACCATCG ATGAAGGAAT ATCTCTTCTC AGAAGGAAAG AATAACCAAC ACCCTGAGGT | 1200 |
| | TGTGTGTGGA AGGTGATCTG GCTCTGGACA GGCACTATCT GGGTTTTGGG GGGACGCTAT | 1260 |
| | AGGATGTGGG GAAGTTAGGA ACTGGTGTCT TCAGGGGCCA CACCAACCTT CTGAGGAGCT | 1320 |
| | GTTGAGGTAC CTCCAAGGAC CGGCCTTTGC ACCTCCATGG AAACGAAGCA CCATCATTCC | 1380 |
| | CGTTGAACGT CACASCTTTA ACCCACTAAC TGGCTAATTA GCATGGCCAC ATCTGAGCCC | 1440 |
| | CGAATCTGAC ATTAGATGAG AGAACAGGGC TGAAGCTGTG TCCTCATGAG GGCTGGATGC | 1500 |
| | TCTCGTTGAC CCTCACAGGA GCATCTCCTC AACTCTGAGT GTTAAGCGTT GAGCCACCAA | 1560 |
| | GCTGGTGGCT CTGTGTGCTC TGATCCGAGC TCAGGGGGGT GGTTTTCCCA TCTCAGGTGT | 1620 |
| | GTTGCAGTGT CTGCTGGAGA CATTGAGGCA GGCACTGCCA AAACATCAAC CTGCCAGCTG | 1680 |
| | GCCTTGTGAG GAGCTGGAAA CACATGTTCC CCTTGGGGGT GGTGGATGAA CAAAGAGAAA | 1740 |
| | GAGGGTTTGG AAGCCAGATC TATGCCACAA GAACCCCCTT TACCCCCATG ACCAACATCG | 1800 |
| | CAGACACATG TGCTGGCCAC CTGCTGAGCC CCAAGTGGAA CGAGACAAGC AGCCCTTAGC | 1860 |
| | CCTTCCCCTC TGCAGCTTCC AGGCTGGCGT GCAGCATCAG CATCCCTAGA AAGCCATGTG | 1920 |
| | CAGCCACCAG TCCASTGGGC AGGCAGATGT TCCTAATAAA GCTTCTGTTC CGTGCTTGTC | 1980 |
| | CCTGTGGAAG TATCTTGGTT GTGACAGAGT CAAGGGTGTG TGCAGCATTG TTGGCTGTTC | 2040 |
| | CTGCAGTAGA ATGGGGGCAG CACCTCCTAA GAAGGCACCT CTCTGGGTTG AAGGGCAGTG | 2100 |
| | TTCCCTGGGG CTTTAACTCC TGCTAGAACA GTCTCTTGAG GCACAGAAAC TCCTGTTCAT | 2160 |
| | GCCCATACCC CTGGCCAAGG AAGATCCCTT TGTCCACAAG TAAAAGGAAA TGCTCCTCCA | 2220 |
| | GGGAGTCTCA GCTTCACCCT GAGGTGAGCA TCATCTTCTG GGTTAGGCCT TGCCTAGGCA | 2280 |
| | TAGCCCTGCC TCAAGCTATG TGAGCTCACC AGTCCCTCCC CAAATGCTTT CCATGAGTTG | 2340 |
| | CAGTTTTTTC CTAGTCTGTT TTCCCTCCTT GGAGACAGGG CCCTGTCGGT TTATTCACTG | 2400 |
| | TATGTCCTTG GTGCCTGGAG CCTACTAAAT GCTCAATAAA TAATGATCAC AGGAATGAA | 2459 |
| SEQ ID NO: 647 | AGAGACAGAA GGTGGATAGA CAAATCTCCA CCTTCAGACT GGTAGGCTCC TCCAGAAGCC | 60 |
| CXCR2 variant 1 | ATCAGACAGG AAGATGTGAA AATCCCCAGC ACTCATCCCA GAATCACTAA GTGGCACCTG | 120 |
| domain | TCCTGGGCCA AAGTCCCAGG ACAGACCTCA TTGTTCCTCT GTGGGAATAC CTCCCCAGGA | 180 |
| | GGGCATCCTG GATTTCCCCC TTGCAACCCA GGTCAGAAGT TTCATCGTCA AGGTTGTTTC | 240 |
| | ATCTTTTTTT TCCTGTCTAA CAGCTCTGAC TACCACCCAA CCTTGAGGCA CAGTGAAGAC | 300 |
| | ATCGGTGGCC ACTCCAATAA CAGCAGGTCA CAGCTGCTCT TCTGGAGGTG TCCTACAGGT | 360 |
| | GAAAAGCCCA GCGACCCAGT CAGGATTTAA GTTTACCTCA AAAATGGAAG ATTTTAACAT | 420 |
| | GGAGAGTGAC AGCTTTGAAG ATTTCTGGAA AGGTGAAGAT CTTAGTAATT ACAGTTACAG | 480 |
| | CTCTACCCTG CCCCCTTTTC TACTAGATGC CGCCCCATGT GAACCAGAAT CCCTGGAAAT | 540 |
| | CAACAAGTAT TTTGTGGTCA TTATCTATGC CCTGGTATTC CTGCTGAGCC TGCTGGGAAA | 600 |
| | CTCCCTCGTG ATGCTGGTCA TCTTATACAG CAGGGTCGGC CGCTCCGTCA CTGATGTCTA | 660 |
| | CCTGCTGAAC CTAGCCTTGG CCGACCTACT CTTTGCCCTG ACCTTGCCCA TCTGGGCCGC | 720 |
| | CTCCAAGGTG AATGGCTGGA TTTTTGGCAC ATTCCTGTGC AAGGTGGTCT CACTCCTGAA | 780 |
| | GGAAGTCAAC TTCTATAGTG GCATCCTGCT ACTGGCCTGC ATCAGTGTGG ACCGTTACCT | 840 |
| | GGCCATTGTC CATGCCACAC GCACACTGAC CCAGAAGCGC TACTTGGTCA AATTCATATG | 900 |
| | TCTCAGCATC TGGGGTCTGT CCTTGCTCCT GGCCCTGCCT GTCTTACTTT TCCGAAGGAC | 960 |
| | CGTCTACTCA TCCAATGTTA GCCCAGCCTG CTATGAGGAC ATGGGCAACA ATACAGCAAA | 1020 |
| | CTGGCGGATG CTGTTACGGA TCCTGCCCCA GTCCTTTGGC TTCATCGTGC CACTGCTGAT | 1080 |
| | CATGCTGTTC TGCTACGGAT TCACCCTGCG TACGCTGTTT AAGGCCCACA TGGGGCAGAA | 1140 |
| | GCACCGGGCC ATGCGGGTCA TCTTTGCTGT CGTCCTCATC TTCCTGCTCT GCTGGCTGCC | 1200 |
| | CTACAACCTG GTCCTGCTGG CAGACACCCT CATGAGGACC CAGGTGATCC AGGAGACCTG | 1260 |
| | TGAGCGCCGC AATCACATCG ACCGGGCTCT GGATGCCACC GAGATTCTGG GCATCCTTCA | 1320 |
| | CAGCTGCCTC AACCCCCTCA TCTACGCCTT CATTGGCCAG AAGTTTCGCC ATGGACTCCT | 1380 |
| | CAAGATTCTA GCTATACATG GCTTGATCAG CAAGGACTCC CTGCCCAAAG ACAGCAGGCC | 1440 |
| | TTCCTTTGTT GGCTCTTCTT CAGGGCACAC TTCCACTACT CTCTAAGACC TCCTGCCTAA | 1500 |
| | GTGCAGCCCC GTGGGGTTCC TCCCTTCTCT TCACAGTCAC ATTCCAAGCC TCATGTCCAC | 1560 |
| | TGGTTCTTCT TGGTCTCAGT GTCAATGCAG CCCCCATTGT GGTCACAGGA AGTAGAGGAG | 1620 |
| | GCCACGTTCT TACTAGTTTC CCTTGCATGG TTTAGAAAGC TTGCCCTGGT GCCTCACCCC | 1680 |
| | TTGCCATAAT TACTATGTCA TTTGCTGGAG CTCTGCCCAT CCTGCCCCTG AGCCCATGGC | 1740 |
| | ACTCTATGTT CTAAGAAGTG AAAATCTACA CTCCAGTGAG ACAGCTCTGC ATACTCATTA | 1800 |
| | GGATGGCTAG TATCAAAAGA AAGAAAATCA GGCTGGCCAA CGGGGTGAAA CCCTGTCTCT | 1860 |
| | ACTAAAAATA CAAAAAAAAA AAAAAATTAG CCGGGCGTGG TGGTGAGTGC CTGTAATCAC | 1920 |
| | AGCTACTTGG GAGGCTGAGA TGGGAGAATC ACTTGAACCC GGGAGGCAGA GGTTGCAGTG | 1980 |
| | AGCCGAGATT GTGCCCCTGC ACTCCAGCCT GAGCGACAGT GAGACTCTGT CTCAGTCCAT | 2040 |
| | GAAGATGTAG AGGAGAAACT GGAACTCTCG AGCGTTGCTG GGGGGGATTG TAAAATGGTG | 2100 |
| | TGACCACTGC AGAAGACAGT ATGGCAGCTT TCCTCAAAAC TTCAGACATA GAATTAACAC | 2160 |
| | ATGATCCTGC AATTCCACTT ATAGGAATTG ACCCACAAGA AATGAAAGCA GGGACTTGAA | 2220 |
| | CCCATATTTG TACACCAATA TTCATAGCAG CTTATTCACA AGACCCAAAA GGCAGAAGCA | 2280 |
| | ACCCAAATGT TCATCAATGA ATGAATGAAT GGCTAAGCAA AATGTGATAT GTACCTAACG | 2340 |
| | AAGTATCCTT CAGCCTGAAA GAGGAATGAA GTACTCATAC ATGTTACAAC ACGGACGAAC | 2400 |
| | CTTGAAAACT TTATGCTAAG TGAAATAAGC CAGACATCAA CAGATAAATA GTTTATGATT | 2460 |
| | CCACCTACAT GAGGTACTGA GAGTGAACAA ATTTACAGAG ACAGAAAGCA GAACAGTGAT | 2520 |
| | TACCAGGGAC TGAGGGGAGG GGAGCATGGG AAGTGACGGT TTAATGGGCA CAGGGTTTAT | 2580 |
| | GTTTAGGATG TTGAAAAAGT TCTGCAGATA AACAGTAGTG ATAGTTGTAC CGCAATGTGA | 2640 |
| | CTTAATGCCA CTAAATTGAC ACTTAAAAAT GGTTTAAATG GTCAATTTTG TTATGTATAT | 2700 |
| | TTTATATCAA TTTAAAAAAA AACCTGAGCC CCAAAAGGTA TTTTAATCAC CAAGGCTGAT | 2760 |
| | TAAACCAAGG CTAGAACCAC CTGCCTATAT TTTTTGTTAA ATGATTTCAT TCAATATCTT | 2820 |
| | TTTTTTAATA AACCATTTTT ACTTGGGTGT TTA | 2853 |
| SEQ ID NO: 648 | AGTGGTGATA GCTGAGAATA TGCAGCCGTT TTCTCCTTCC TGGGTACAGT GCTATTCTGC | 60 |
| CXCR2 variant 2 | CTAGAGCTCT GACTACCACC CAACCTTGAG GCACAGTGAA GACATCGGTG GCCACTCCAA | 120 |
| domain | TAACAGCAGG TCACAGCTGC TCTTCTGGAG GTGTCCTACA GGTGAAAAGC CCAGCGACCC | 180 |
| | AGTCAGGATT TAAGETTACC TCAAAAATGG AAGATTTTAA CATGGAGAGT GACAGCTTTG | 240 |
| | AAGATTTCTG GAAAGGTGAA GATCTTAGTA ATTACAGTTA CAGCTCTACC CTGCCCCCTT | 300 |
| | TTCTACTAGA TGCCGCCCCA TGTGAACCAG AATCCCTGGA AATCAACAAG TATTTTGTGG | 360 |
| | TCATTATCTA TGCCCTGGTA TTCCTGCTGA GCCTGCTGGG AAACTCCCTC GTGATGCTGG | 420 |
| | TCATCTTATA CAGCAGGGTC GGCCGCTCCG TCACTGATGT CTACCTGCTG AACCTAGCCT | 480 |
| | TGGCCGACCT ACTCETTGCC CTGACCTTGC CCATCTGGGC CGCCTCCAAG GTGAATGGCT | 540 |
| | GGATTTTTGG CACATTCCTG TGCAAGGTGG TCTCACTCCT GAAGGAAGTC AACTTCTATA | 600 |
| | GTGGCATCCT GCTACTGGCC TGCATCAGTG TGGACCGTTA CCTGGCCATT GTCCATGCCA | 660 |
| | CACGCACACT GACCCAGAAG CGCTACTTGG TCAAATTCAT ATGTCTCAGC ATCTGGGGTC | 720 |
| | TGTCCTTGCT CCTGGCCCTG CCTGTCTTAC TTTTCCGAAG GACCGTCTAC TCATCCAATG | 780 |
| | TTAGCCCAGC CTGCTATGAG GACATGGGCA ACAATACAGC AAACTGGCGG ATGCTGTTAC | 840 |
| | GGATCCTGCC CCAGTCCTTT GGCTTCATCG TGCCACTGCT GATCATGCTG TTCTGCTACG | 900 |
| | GATTCACCCT GCGTACGCTG TTTAAGGCCC ACATGGGGCA GAAGCACCGG GCCATGCGGG | 960 |
| | TCATCTTTGC TGTCGTCCTC ATCTTCCTGC TCTGCTGGCT GCCCTACAAC CTGGTCCTGC | 1020 |
| | TGGCAGACAC CCTCATGAGG ACCCAGGTGA TCCAGGAGAC CTGTGAGCGC CGCAATCACA | 1080 |
| | TCGACCGGGC TCTGGATGCC ACCGAGATTC TGGGCATCCT TCACAGCTGC CTCAACCCCC | 1140 |
| | TCATCTACGC CTTCATTGGC CAGAAGTTTC GCCATGGACT CCTCAAGATT CTAGCTATAC | 1200 |
| | ATGGCTTGAT CAGCAAGGAC TCCCTGCCCA AAGACAGCAG GCCTTCCTTT GTTGGCTCTT | 1260 |
| | CTTCAGGGCA CACTTCCACT ACTCTCTAAG ACCTCCTGCC TAAGTGCAGC CCCGTGGGGT | 1320 |
| | TCCTCCCTTC TCTTCACAGT CACATTCCAA GCCTCATGTC CACTGGTTCT TCTTGGTCTC | 1380 |
| | AGTGTCAATG CAGCCCCCAT TGTGGTCACA GGAAGTAGAG GAGGCCACGT TCTTACTAGT | 1440 |
| | TTCCCTTGCA TGGTTTAGAA AGCTTGCCCT GGTGCCTCAC CCCTTGCCAT AATTACTATG | 1500 |
| | TCATTTGCTG GAGCTCTGCC CATCCTGCCC CTGAGCCCAT GGCACTCTAT GTTCTAAGAA | 1560 |
| | GTGAAAATCT ACACTCCAGT GAGACAGCTC TGCATACTCA TTAGGATGGC TAGTATCAAA | 1620 |
| | AGAAAGAAAA TCAGGCTGGC CAACGGGGTG AAACCCTGTC TCTACTAAAA ATACAAAAAA | 1680 |
| | AAAAAAAAAT TAGCCGGGCG TGGTGGTGAG TGCCTGTAAT CACAGCTACT TGGGAGGCTG | 1740 |
| | AGATGGGAGA ATCACTTGAA CCCGGGAGGC AGAGGTTGCA GTGAGCCGAG ATTGTGCCCC | 1800 |
| | TGCACTCCAG CCTGAGCGAC AGTGAGACTC TGTCTCAGTC CATGAAGATG TAGAGGAGAA | 1860 |
| | ACTGGAACTC TCGAGCGTTG CTGGGGGGGA TTGTAAAATG GTGTGACCAC TGCAGAAGAC | 1920 |
| | AGTATGGCAG CTTTCCTCAA AACTTCAGAC ATAGAATTAA CACATGATCC TGCAATTCCA | 1980 |
| | CTTATAGGAA TTGACCCACA AGAAATGAAA GCAGGGACTT GAACCCATAT TTGTACACCA | 2040 |
| | ATATTCATAG CAGCTTATTC ACAAGACCCA AAAGGCAGAA GCAACCCAAA TGTTCATCAA | 2100 |
| | TGAATGAATG AATGGCTAAG CAAAATGTGA TATGTACCTA ACGAAGTATC CTTCAGCCTG | 2160 |
| | AAAGAGGAAT GAAGTACTCA TACATGTTAC AACACGGACG AACCTTGAAA ACTTTATGCT | 2220 |
| | AAGTGAAATA AGCCAGACAT CAACAGATAA ATAGTTTATG ATTCCACCTA CATGAGGTAC | 2280 |
| | TGAGAGTGAA CAAATTTACA GAGACAGAAA GCAGAACAGT GATTACCAGG GACTGAGGGG | 2340 |
| | AGGGGAGCAT GGGAAGTGAC GGTTTAATGG GCACAGGGTT TATGTTTAGG ATGTTGAAAA | 2400 |
| | AGTTCTGCAG ATAAACAGTA GTGATAGTTG TACCGCAATG TGACTTAATG CCACTAAATT | 2460 |
| | GACACTTAAA AATGGTTTAA ATGGTCAATT TTGTTATGTA TATTTTATAT CAATTTAAAA | 2520 |
| | AAAAACCTGA GCCCCAAAAG GTATTTTAAT CACCAAGGCT GATTAAACCA AGGCTAGAAC | 2580 |
| | CACCTGCCTA TATTTTTTGT TAAATGATTT CATTCAATAT CTTTTTTTTA ATAAACCATT | 2640 |
| | TTTACTTGGG TGTTTA | 2656 |
| SEQ ID NO: 649 | ACAAGCACCA AAGCAGAGGG GCAGGCAGCA CACCACCCAG CAGCCAGAGC ACCAGCCCAG | 60 |
| CXCR3 variant 1 | CCATGGTCCT TGAGGTGAGT GACCACCAAG TGCTAAATGA CGCCGAGGTT GCCGCCCTCC | 120 |
| domain | TGGAGAACTT CAGCTCTTCC TATGACTATG GAGAAAACGA GAGTGACTCG TGCTGTACCT | 180 |
| | CCCCGCCCTG CCCACAGGAC TTCAGCCTGA ACTTCGACCG GGCCTTCCTG CCAGCCCTCT | 240 |
| | ACAGCCTCCT CTTTCTGCTG GGGCTGCTGG GCAACGGCGC GGTGGCAGCC GTGCTGCTGA | 300 |
| | GCCGGCGGAC AGCCCTGAGC AGCACCGACA CCTTCCTGCT CCACCTAGCT GTAGCAGACA | 360 |
| | CGCTGCTGGT GCTGACACTG CCGCTCTGGG CAGTGGACGC TGCCGTCCAG TGGGTCTTTG | 420 |
| | GCTCTGGCCT CTGCAAAGTG GCAGGTGCCC TCTTCAACAT CAACTTCTAC GCAGGAGCCC | 480 |
| | TCCTGCTGGC CTGCATCAGC TTTGACCGCT ACCTGAACAT AGTTCATGCC ACCCAGCTCT | 540 |
| | ACCGCCGGGG GCCCCCGGCC CGCGTGACCC TCACCTGCCT GGCTGTCTGG GGGCTCTGCC | 600 |
| | TGCTTTTCGC CCTCCCAGAC TTCATCTTCC TGTCGGCCCA CCACGACGAG CGCCTCAACG | 660 |
| | CCACCCACTG CCAATACAAC TTCCCACAGG TGGGCCGCAC GGCTCTGCGG GTGCTGCAGC | 720 |
| | TGGTGGCTGG CTTTCTGCTG CCCCTGCTGG TCATGGCCTA CTGCTATGCC CACATCCTGG | 780 |
| | CCGTGCTGCT GGTTTCCAGG GGCCAGCGGC GCCTGCGGGC CATGCGGCTG GTGGTGGTGG | 840 |
| | TCGTGGTGGC CTTTGCCCTC TGCTGGACCC CCTATCACCT GGTGGTGCTG GTGGACATCC | 900 |
| | TCATGGACCT GGGCGCTTTG GCCCGCAACT GTGGCCGAGA AAGCAGGGTA GACGTGGCCA | 960 |
| | AGTCGGTCAC CTCAGGCCTG GGCTACATGC ACTGCTGCCT CAACCCGCTG CTCTATGCCT | 1020 |
| | TTGTAGGGGT CAAGTTCCGG GAGCGGATGT GGATGCTGCT CTTGCGCCTG GGCTGCCCCA | 1080 |
| | ACCAGAGAGG GCTCCAGAGG CAGCCATCGT CTTCCCGCCG GGATTCATCC TGGTCTGAGA | 1140 |
| | CCTCAGAGGC CTCCTACTCG GGCTTGTGAG GCCGGAATCC GGGCTCCCCT TTCGCCCACA | 1200 |
| | GTCTGACTTC CCCGCATTCC AGGCTCCTCC CTCCCTCTGC CGGCTCTGGC TCTCCCCAAT | 1260 |
| | ATCCTCGCTC CCGGGACTCA CTGGCAGCCC CAGCACCACC AGGTCTCCCG GGAAGCCACC | 1320 |
| | CTCCCAGCTC TGAGGACTGC ACCATTGCTG CTCCTTAGCT GCCAAGCCCC ATCCTGCCGC | 1380 |
| | CCGAGGTGGC TGCCTGGAGC CCCACTGCCC TTCTCATTTG GAAACTAAAA CTTCATCTTC | 1440 |
| | CCCAAGTGCG GGGAGTACAA GGCATGGCGT AGAGGGTGCT GCCCCATGAA GCCACAGCCC | 1500 |
| | AGGCCTCCAG CTCAGCAGTG ACTGTGGCCA TGGTCCCCAA GACCTCTATA TTTGCTCTTT | 1560 |
| | TATTTTTATG TCTAAAATCC TGCTTAAAAC TTTTCAATAA ACAAGATCGT CAGGA | 1615 |
| SEQ ID NO: 650 | ACAAGCACCA AAGCAGAGGG GCAGGCAGCA CACCACCCAG CAGCCAGAGC ACCAGCCCAG | 60 |
| CXCR3 variant 2 | CCATGGTCCT TGAGGGGTCC CTGGGCCGAT GGGATCACGC AGAAGAATGC GAGAGAAGCA | 120 |
| domain | GCCTTTGAGA AGGGAAGTCA CTATCCCAGA GCCCAGGCTG AGCGGATGGA GTTGAGGAAG | 180 |
| | TACGGCCCTG GAAGACTGGC GGGGACAGTT ATAGGAGGAG CTGCTCAGAG TAAATCACAG | 240 |
| | ACTAAATCAG ACTCAATCAC AAAAGAGTTC CTGCCAGGCC TTTACACAGC CCCTTCCTCC | 300 |
| | CCGTTCCCGC CCTCACAGGT GAGTGACCAC CAAGTGCTAA ATGACGCCGA GGTTGCCGCC | 360 |
| | CTCCTGGAGA ACTTCAGCTC TTCCTATGAC TATGGAGAAA ACGAGAGTGA CTCGTGCTGT | 420 |
| | ACCTCCCCGC CCTGCCCACA GGACTTCAGC CTGAACTTCG ACCGGGCCTT CCTGCCAGCC | 480 |
| | CTCTACAGCC TCCTCTTTCT GCTGGGGCTG CTGGGCAACG GCGCGGTGGC AGCCGTGCTG | 540 |
| | CTGAGCCGGC GGACAGCCCT GAGCAGCACC GACACCTTCC TGCTCCACCT AGCTGTAGCA | 600 |
| | GACACGCTGC TGGTGCTGAC ACTGCCGCTC TGGGCAGTGG ACGCTGCCGT CCAGTGGGTC | 660 |
| | TTTGGCTCTG GCCTCTGCAA AGTGGCAGGT GCCCTCTTCA ACATCAACTT CTACGCAGGA | 720 |
| | GCCCTCCTGC TGGCCTGCAT CAGCTTTGAC CGCTACCTGA ACATAGTTCA TGCCACCCAG | 780 |
| | CTCTACCGCC GGGGGCCCCC GGCCCGCGTG ACCCTCACCT GCCTGGCTGT CTGGGGGCTC | 840 |
| | TGCCTGCTTT TCGCCCTCCC AGACTTCATC TTCCTGTCGG CCCACCACGA CGAGCGCCTC | 900 |
| | AACGCCACCC ACTGCCAATA CAACTTCCCA CAGGTGGGCC GCACGGCTCT GCGGGTGCTG | 960 |
| | CAGCTGGTGG CTGGCTTTCT GCTGCCCCTG CTGGTCATGG CCTACTGCTA TGCCCACATC | 1020 |
| | CTGGCCGTGC TGCTGGTTTC CAGGGGCCAG CGGCGCCTGC GGGCCATGCG GCTGGTGGTG | 1080 |
| | GTGGTCGTGG TGGCCTTTGC CCTCTGCTGG ACCCCCTATC ACCTGGTGGT GCTGGTGGAC | 1140 |
| | ATCCTCATGG ACCTGGGCGC TTTGGCCCGC AACTGTGGCC GAGAAAGCAG GGTAGACGTG | 1200 |
| | GCCAAGTCGG TCACCTCAGG CCTGGGCTAC ATGCACTGCT GCCTCAACCC GCTGCTCTAT | 1260 |
| | GCCTTTGTAG GGGTCAAGTT CCGGGAGCGG ATGTGGATGC TGCTCTTGCG CCTGGGCTGC | 1320 |
| | CCCAACCAGA GAGGGCTCCA GAGGCAGCCA TCGTCTTCCC GCCGGGATTC ATCCTGGTCT | 1380 |
| | GAGACCTCAG AGGCCTCCTA CTCGGGCTTG TGAGGCCGGA ATCCGGGCTC CCCTTTCGCC | 1440 |
| | CACAGTCTGA CTTCCCCGCA TTCCAGGCTC CTCCCTCCCT CTGCCGGCTC TGGCTCTCCC | 1500 |
| | CAATATCCTC GCTCCCGGGA CTCACTGGCA GCCCCAGCAC CACCAGGTCT CCCGGGAAGC | 1560 |
| | CACCCTCCCA GCTCTGAGGA CTGCACCATT GCTGCTCCTT AGCTGCCAAG CCCCATCCTG | 1620 |
| | CCGCCCGAGG TGGCTGCCTG GAGCCCCACT GCCCTTCTCA TTTGGAAACT AAAACTTCAT | 1680 |
| | CTTCCCCAAG TGCGGGGAGT ACAAGGCATG GOGTAGAGGG TGCTGCCCCA TGAAGCCACA | 1740 |
| | GCCCAGGCCT CCAGCTCAGC AGTGACTGTG GCCATGGTCC CCAAGACCTC TATATTTGCT | 1800 |
| | CTTTTATTTT TATGTCTAAA ATCCTGCTTA AAACTTTTCA ATAAACAAGA TCGTCAGGA | 1859 |
| SEQ ID NO: 651 | CTTCCCTCTA GTGGGCGGGG CAGAGGAGTT AGCCAAGATG TGACTTTGAA ACCCTCAGCG | 60 |
| CXCR4 variant 1 | TCTCAGTGCC CTTTTGTTCT AAACAAAGAA TTTTGTAATT GGTTCTACCA AAGAAGGATA | 120 |
| domain | TAATGAAGTC ACTASGGGAA AAGATGGGGA GGAGAGTTGT AGGATTCTAC ATTAATTCTC | 180 |
| | TTGTGCCCTT AGCCCACTAC TTCAGAATTT CCTGAAGAAA GCAAGCCTGA ATTGGTTTTT | 240 |
| | TAAATTGCTT TAAAAATTTT TTTTAACTGG GTTAATGCTT GCTGAATTGG AAGTGAATGT | 300 |
| | CCATTCCTTT GCCTCTTTTG CAGATATACA CTTCAGATAA CTACACCGAG GAAATGGGCT | 360 |
| | CAGGGGACTA TGACTCCATG AAGGAACCCT GTTTCCGTGA AGAAAATGCT AATTTCAATA | 420 |
| | AAATCTTCCT GCCCACCATC TACTCCATCA TCTTCTTAAC TGGCATTGTG GGCAATGGAT | 480 |
| | TGGTCATCCT GGTCATGGGT TACCAGAAGA AACTGAGAAG CATGACGGAC AAGTACAGGC | 540 |
| | TGCACCTGTC AGTGGCCGAC CTCCTCTTTG TCATCACGCT TCCCTTCTGG GCAGTTGATG | 600 |
| | CCGTGGCAAA CTGGTACTTT GGGAACTTCC TATGCAAGGC AGTCCATGTC ATCTACACAG | 660 |
| | TCAACCTCTA CAGCAGTGTC CTCATCCTGG CCTTCATCAG TCTGGACCGC TACCTGGCCA | 720 |
| | TCGTCCACGC CACCAACAGT CAGAGGCCAA GGAAGCTGTT GGCTGAAAAG GTGGTCTATG | 780 |
| | TTGGCGTCTG GATCCCTGCC CTCCTGCTGA CTATTCCCGA CTTCATCTTT GCCAACGTCA | 840 |
| | GTGAGGCAGA TGACAGATAT ATCTGTGACC GCTTCTACCC CAATGACTTG TGGGTGGTTG | 900 |
| | TGTTCCAGTT TCAGCACATC ATGGTTGGCC TTATCCTGCC TGGTATTGTC ATCCTGTCCT | 960 |
| | GCTATTGCAT TATCATCTCC AAGCTGTCAC ACTCCAAGGG CCACCAGAAG CGCAAGGCCC | 1020 |
| | TCAAGACCAC AGTCATCCTC ATCCTGGCTT TCTTCGCCTG TTGGCTGCCT TACTACATTG | 1080 |
| | GGATCAGCAT CGACTCCTTC ATCCTCCTGG AAATCATCAA GCAAGGGTGT GAGTTTGAGA | 1140 |
| | ACACTGTGCA CAAGEGGATT TCCATCACCG AGGCCCTAGC TTTCTTCCAC TGTTGTCTGA | 1200 |
| | ACCCCATCCT CTATGCTTTC CTTGGAGCCA AATTTAAAAC CTCTGCCCAG CACGCACTCA | 1260 |
| | CCTCTGTGAG CAGAGGGTCC AGCCTCAAGA TCCTCTCCAA AGGAAAGCGA GGTGGACATT | 1320 |
| | CATCTGTTTC CACTGAGTCT GAGTCTTCAA GTTTTCACTC CAGCTAACAC AGATGTAAAA | 1380 |
| | GACTTTTTTT TATACGATAA ATAACTTTTT TTTAAGTTAC ACATTTTTCA GATATAAAAG | 1440 |
| | ACTGACCAAT ATTGTACAGT TTTTATTGCT TGTTGGATTT TTGTCTTGTG TTTCTTTAGT | 1500 |
| | TTTTGTGAAG TTTAATTGAC TTATTTATAT AAATTTTTTT TGTTTCATAT TGATGTGTGT | 1560 |
| | CTAGGCAGGA CCTGTGGCCA AGTTCTTAGT TGCTGTATGT CTCGTGGTAG GACTGTAGAA | 1620 |
| | AAGGGAACTG AACATTCCAG AGCGTGTAGT GAATCACGTA AAGCTAGAAA TGATCCCCAG | 1680 |
| | CTGTTTATGC ATAGATAATC TCTCCATTCC CGTGGAACGT TTTTCCTGTT CTTAAGACGT | 1740 |
| | GATTTTGCTG TAGAAGATGG CACTTATAAC CAAAGCCCAA AGTGGTATAG AAATGCTGGT | 1800 |
| | TTTTCAGTTT TCAGGAGTGG GTTGATTTCA GCACCTACAG TGTACAGTCT TGTATTAAGT | 1860 |
| | TGTTAATAAA AGTACATGTT AAACTTAAAA AAAAAAAAAA AAAA | 1904 |
| SEQ ID NO: 652 | AGTTTGTTGG CTGCGGCAGC AGGTAGCAAA GTGACGCCGA GGGCCTGAGT GCTCCAGTAG | 60 |
| CXCR4 variant 2 | CCACCGCATC TGGAGAACCA GCGGTTACCA TGGAGGGGAT CAGTATATAC ACTTCAGATA | 120 |
| domain | ACTACACCGA GGAAATGGGC TCAGGGGACT ATGACTCCAT GAAGGAACCC TGTTTCCGTG | 180 |
| | AAGAAAATGC TAATTTCAAT AAAATCTTCC TGCCCACCAT CTACTCCATC ATCTTCTTAA | 240 |
| | CTGGCATTGT GGGCAATGGA TTGGTCATCC TGGTCATGGG TTACCAGAAG AAACTGAGAA | 300 |
| | GCATGACGGA CAAGTACAGG CTGCACCTGT CAGTGGCCGA CCTCCTCTTT GTCATCACGC | 360 |
| | TTCCCTTCTG GGCAGTTGAT GCCGTGGCAA ACTGGTACTT TGGGAACTTC CTATGCAAGG | 420 |
| | CAGTCCATGT CATCTACACA GTCAACCTCT ACAGCAGTGT CCTCATCCTG GCCTTCATCA | 480 |
| | GTCTGGACCG CTACCTGGCC ATCGTCCACG CCACCAACAG TCAGAGGCCA AGGAAGCTGT | 540 |
| | TGGCTGAAAA GGTGGTCTAT GTTGGCGTCT GGATCCCTGC CCTCCTGCTG ACTATTCCCG | 600 |
| | ACTTCATCTT TGCCAACGTC AGTGAGGCAG ATGACAGATA TATCTGTGAC CGCTTCTACC | 660 |
| | CCAATGACTT GTGGGTGGTT GTGTTCCAGT TTCAGCACAT CATGGTTGGC CTTATCCTGC | 720 |
| | CTGGTATTGT CATCCTGTCC TGCTATTGCA TTATCATCTC CAAGCTGTCA CACTCCAAGG | 780 |
| | GCCACCAGAA GCGCAAGGCC CTCAAGACCA CAGTCATCCT CATCCTGGCT TTCTTCGCCT | 840 |
| | GTTGGCTGCC TTACTACATT GGGATCAGCA TCGACTCCTT CATCCTCCTG GAAATCATCA | 900 |
| | AGCAAGGGTG TGAGETTGAG AACACTGTGC ACAAGTGGAT TTCCATCACC GAGGCCCTAG | 960 |
| | CTTTCTTCCA CTGTIGTCTG AACCCCATCC TCTATGCTTT CCTTGGAGCC AAATTTAAAA | 1020 |
| | CCTCTGCCCA GCACGCACTC ACCTCTGTGA GCAGAGGGTC CAGCCTCAAG ATCCTCTCCA | 1080 |
| | AAGGAAAGCG AGGTGGACAT TCATCTGTTT CCACTGAGTC TGAGTCTTCA AGTTTTCACT | 1140 |
| | CCAGCTAACA CAGATGTAAA AGACTTTTTT TTATACGATA AATAACTTTT TTTTAAGTTA | 1200 |
| | CACATTTTTC AGATATAAAA GACTGACCAA TATTGTACAG TTTTTATTGC TTGTTGGATT | 1260 |
| | TTTGTCTTGT GTTTCTTTAG TTTTTGTGAA GTTTAATTGA CTTATTTATA TAAATTTTTT | 1320 |
| | TTGTTTCATA TTGATGTGTG TCTAGGCAGG ACCTGTGGCC AAGTTCTTAG TTGCTGTATG | 1380 |
| | TCTCGTGGTA GGACTGTAGA AAAGGGAACT GAACATTCCA GAGCGTGTAG TGAATCACGT | 1440 |
| | AAAGCTAGAA ATGATCCCCA GCTGTTTATG CATAGATAAT CTCTCCATTC CCGTGGAACG | 1500 |
| | TTTTTCCTGT TCTTAAGACG TGATTTTGCT GTAGAAGATG GCACTTATAA CCAAAGCCCA | 1560 |
| | AAGTGGTATA GAAATGCTGG TTTTTCAGTT TTCAGGAGTG GGTTGATTTC AGCACCTACA | 1620 |
| | GTGTACAGTC TTGTATTAAG TTGTTAATAA AAGTACATGT TAAACTTA | 1668 |
| SEQ ID NO: 653 | AGTTTGTTGG CTGCGGCAGC AGGTAGCAAA GTGACGCCGA GGGCCTGAGT GCTCCAGTAG | 60 |
| CXCR4 variant 3 | CCACCGCATC TGGAGAACCA GCGGTTACCA TGGAGGGGAT CAGTGAAAAT GCCCCGCTCC | 120 |
| domain | CTAACGTCCC AAACGCGCCA AGTGATAAAC ACGAGGATGG CAAGAGACCC ACACACCGGA | 180 |
| | GGAGCGCCCG CTTGGGGGAG GAGGTGCCGT TTGTTCATTT TCTGACACTC CCGCCCAATA | 240 |
| | TACCCCAAGC ACCGAAGGGC CTTCGTTTTA AGACCGCATT CTCTTTACCC ACTACAAGTT | 300 |
| | GCTTGAAGCC CAGAATGATA TACACTTCAG ATAACTACAC CGAGGAAATG GGCTCAGGGG | 360 |
| | ACTATGACTC CATGAAGGAA CCCTGTTTCC GTGAAGAAAA TGCTAATTTC AATAAAATCT | 420 |
| | TCCTGCCCAC CATCTACTCC ATCATCTTCT TAACTGGCAT TGTGGGCAAT GGATTGGTCA | 480 |
| | TCCTGGTCAT GGGTTACCAG AAGAAACTGA GAAGCATGAC GGACAAGTAC AGGCTGCACC | 540 |
| | TGTCAGTGGC CGACCTCCTC TTTGTCATCA CGCTTCCCTT CTGGGCAGTT GATGCCGTGG | 600 |
| | CAAACTGGTA CTTTGGGAAC TTCCTATGCA AGGCAGTCCA TGTCATCTAC ACAGTCAACC | 660 |
| | TCTACAGCAG TGTCCTCATC CTGGCCTTCA TCAGTCTGGA CCGCTACCTG GCCATCGTCC | 720 |
| | ACGCCACCAA CAGTCAGAGG CCAAGGAAGC TGTTGGCTGA AAAGGTGGTC TATGTTGGCG | 780 |
| | TCTGGATCCC TGCCCTCCTG CTGACTATTC CCGACTTCAT CTTTGCCAAC GTCAGTGAGG | 840 |
| | CAGATGACAG ATATATCTGT GACCGCTTCT ACCCCAATGA CTTGTGGGTG GTTGTGTTCC | 900 |
| | AGTTTCAGCA CATCATGGTT GGCCTTATCC TGCCTGGTAT TGTCATCCTG TCCTGCTATT | 960 |
| | GCATTATCAT CTCCAAGCTG TCACACTCCA AGGGCCACCA GAAGCGCAAG GCCCTCAAGA | 1020 |
| | CCACAGTCAT CCTCATCCTG GCTTTCTTCG CCTGTTGGCT GCCTTACTAC ATTGGGATCA | 1080 |
| | GCATCGACTC CTTCATCCTC CTGGAAATCA TCAAGCAAGG GTGTGAGTTT GAGAACACTG | 1140 |
| | TGCACAAGTG GATTTCCATC ACCGAGGCCC TAGCTTTCTT CCACTGTTGT CTGAACCCCA | 1200 |
| | TCCTCTATGC TTTCCTTGGA GCCAAATTTA AAACCTCTGC CCAGCACGCA CTCACCTCTG | 1260 |
| | TGAGCAGAGG GTCCAGCCTC AAGATCCTCT CCAAAGGAAA GCGAGGTGGA CATTCATCTG | 1320 |
| | TTTCCACTGA GTCTGAGTCT TCAAGTTTTC ACTCCAGCTA ACACAGATGT AAAAGACTTT | 1380 |
| | TTTTTATACG ATAAATAACT TTTTTTTAAG TTACACATTT TTCAGATATA AAAGACTGAC | 1440 |
| | CAATATTGTA CAGTTTTTAT TGCTTGTTGG ATTTTTGTCT TGTGTTTCTT TAGTTTTTGT | 1500 |
| | GAAGTTTAAT TGACTTATTT ATATAAATTT TTTTTGTTTC ATATTGATGT GTGTCTAGGC | 1560 |
| | AGGACCTGTG GCCAAGTTCT TAGTTGCTGT ATGTCTCGTG GTAGGACTGT AGAAAAGGGA | 1620 |
| | ACTGAACATT CCAGAGCGTG TAGTGAATCA CGTAAAGCTA GAAATGATCC CCAGCTGTTT | 1680 |
| | ATGCATAGAT AATCTCTCCA TTCCCGTGGA ACGTTTTTCC TGTTCTTAAG ACGTGATTTT | 1740 |
| | GCTGTAGAAG ATGGCACTTA TAACCAAAGC CCAAAGTGGT ATAGAAATGC TGGTTTTTCA | 1800 |
| | GTTTTCAGGA GTGGGTTGAT TTCAGCACCT ACAGTGTACA GTCTTGTATT AAGTTGTTAA | 1860 |
| | TAAAAGTACA TGTTAAACTT A | 1881 |
| SEQ ID NO: 654 | AGTTTGTTGG CTGCGGCAGC AGGTAGCAAA GTGACGCCGA GGGCCTGAGT GCTCCAGTAG | 60 |
| CXCR4 variant 4 | CCACCGCATC TGGAGAACCA GCGGTTACCA TGGAGGGGAT CAGTGAAAAT GCCCCGCTCC | 120 |
| domain | CTAACGTCCC AAACGCGCCA AGTGATAAAC ACGAGGATGG CAAGAGACCC ACACACCGGA | 180 |
| | GGAGCGCCCG CTTGGGGGAG GAGATATACA CTTCAGATAA CTACACCGAG GAAATGGGCT | 240 |
| | CAGGGGACTA TGACTCCATG AAGGAACCCT GTTTCCGTGA AGAAAATGCT AATTTCAATA | 300 |
| | AAATCTTCCT GCCCACCATC TACTCCATCA TCTTCTTAAC TGGCATTGTG GGCAATGGAT | 360 |
| | TGGTCATCCT GGTCATGGGT TACCAGAAGA AACTGAGAAG CATGACGGAC AAGTACAGGC | 420 |
| | TGCACCTGTC AGTGGCCGAC CTCCTCTTTG TCATCACGCT TCCCTTCTGG GCAGTTGATG | 480 |
| | CCGTGGCAAA CTGGTACTTT GGGAACTTCC TATGCAAGGC AGTCCATGTC ATCTACACAG | 540 |
| | TCAACCTCTA CAGCAGTGTC CTCATCCTGG CCTTCATCAG TCTGGACCGC TACCTGGCCA | 600 |
| | TCGTCCACGC CACCAACAGT CAGAGGCCAA GGAAGCTGTT GGCTGAAAAG GTGGTCTATG | 660 |
| | TTGGCGTCTG GATCCCTGCC CTCCTGCTGA CTATTCCCGA CTTCATCTTT GCCAACGTCA | 720 |
| | GTGAGGCAGA TGACAGATAT ATCTGTGACC GCTTCTACCC CAATGACTTG TGGGTGGTTG | 780 |
| | TGTTCCAGTT TCAGCACATC ATGGTTGGCC TTATCCTGCC TGGTATTGTC ATCCTGTCCT | 840 |
| | GCTATTGCAT TATCATCTCC AAGCTGTCAC ACTCCAAGGG CCACCAGAAG CGCAAGGCCC | 900 |
| | TCAAGACCAC AGTCATCCTC ATCCTGGCTT TCTTCGCCTG TTGGCTGCCT TACTACATTG | 960 |
| | GGATCAGCAT CGACTCCTTC ATCCTCCTGG AAATCATCAA GCAAGGGTGT GAGTTTGAGA | 1020 |
| | ACACTGTGCA CAAGTGGATT TCCATCACCG AGGCCCTAGC TTTCTTCCAC TGTTGTCTGA | 1080 |
| | ACCCCATCCT CTATGCTTTC CTTGGAGCCA AATTTAAAAC CTCTGCCCAG CACGCACTCA | 1140 |
| | CCTCTGTGAG CAGAGGGTCC AGCCTCAAGA TCCTCTCCAA AGGAAAGCGA GGTGGACATT | 1200 |
| | CATCTGTTTC CACTGAGTCT GAGTCTTCAA GTTTTCACTC CAGCTAACAC AGATGTAAAA | 1260 |
| | GACTTTTTTT TATACGATAA ATAACTTTTT TTTAAGTTAC ACATTTTTCA GATATAAAAG | 1320 |
| | ACTGACCAAT ATTGTACAGT TTTTATTGCT TGTTGGATTT TTGTCTTGTG TTTCTTTAGT | 1380 |
| | TTTTGTGAAG TTTAATTGAC TTATTTATAT AAATTTTTTT TGTTTCATAT TGATGTGTGT | 1440 |
| | CTAGGCAGGA CCTGTGGCCA AGTTCTTAGT TGCTGTATGT CTCGTGGTAG GACTGTAGAA | 1500 |
| | AAGGGAACTG AACASTCCAG AGCGTGTAGT GAATCACGTA AAGCTAGAAA TGATCCCCAG | 1560 |
| | CTGTTTATGC ATAGATAATC TCTCCATTCC CGTGGAACGT TTTTCCTGTT CTTAAGACGT | 1620 |
| | GATTTTGCTG TAGAAGATGG CACTTATAAC CAAAGCCCAA AGTGGTATAG AAATGCTGGT | 1680 |
| | TTTTCAGTTT TCAGGAGTGG GTTGATTTCA GCACCTACAG TGTACAGTCT TGTATTAAGT | 1740 |
| | TGTTAATAAA AGTACATGTT AAACTTA | 1767 |
| SEQ ID NO: 655 | AGCAGGATTG GAATCTTTTT CTCTGTGAGT CGAGGAGAAA CGACTGGAAA GAGCGTTCCA | 60 |
| CXCR4 variant 5 | GTGGCTGCAT GTGTCTCCCC CTTGAGTCCC GCCGCGCGCG GCGGCTTGCA CGCTGTTTGC | 120 |
| domain | AAACGTAAGA ACATTCTGTG CACAAGTGCA GAGAAGGCGT GCGCGCTGCC TCGGGACTCA | 180 |
| | GACCACCGGT CTCTTCCTTG GGGAAGCGGG GATGTCTTGG AGCGAGTTAC ATTGTCTGAA | 240 |
| | TTTAGAGGCG GAGGGCGGCG TGCCTGGGCT GAGTTCCCAG GAGGAGATTG CGCCCGCTTT | 300 |
| | AACTTCGGGG TTAAGCGCCT GGTGACTGTT CTTGACACTG GATATACACT TCAGATAACT | 360 |
| | ACACCGAGGA AATGGGCTCA GGGGACTATG ACTCCATGAA GGAACCCTGT TTCCGTGAAG | 420 |
| | AAAATGCTAA TTTCAATAAA ATCTTCCTGC CCACCATCTA CTCCATCATC TTCTTAACTG | 480 |
| | GCATTGTGGG CAATGGATTG GTCATCCTGG TCATGGGTTA CCAGAAGAAA CTGAGAAGCA | 540 |
| | TGACGGACAA GTACAGGCTG CACCTGTCAG TGGCCGACCT CCTCTTTGTC ATCACGCTTC | 600 |
| | CCTTCTGGGC AGTTGATGCC GTGGCAAACT GGTACTTTGG GAACTTCCTA TGCAAGGCAG | 660 |
| | TCCATGTCAT CTACACAGTC AACCTCTACA GCAGTGTCCT CATCCTGGCC TTCATCAGTC | 720 |
| | TGGACCGCTA CCTGGCCATC GTCCACGCCA CCAACAGTCA GAGGCCAAGG AAGCTGTTGG | 780 |
| | CTGAAAAGGT GGTCTATGTT GGCGTCTGGA TCCCTGCCCT CCTGCTGACT ATTCCCGACT | 840 |
| | TCATCTTTGC CAACGTCAGT GAGGCAGATG ACAGATATAT CTGTGACCGC TTCTACCCCA | 900 |
| | ATGACTTGTG GGTGGTTGTG TTCCAGTTTC AGCACATCAT GGTTGGCCTT ATCCTGCCTG | 960 |
| | GTATTGTCAT CCTGTCCTGC TATTGCATTA TCATCTCCAA GCTGTCACAC TCCAAGGGCC | 1020 |
| | ACCAGAAGCG CAAGGCCCTC AAGACCACAG TCATCCTCAT CCTGGCTTTC TTCGCCTGTT | 1080 |
| | GGCTGCCTTA CTACATTGGG ATCAGCATCG ACTCCTTCAT CCTCCTGGAA ATCATCAAGC | 1140 |
| | AAGGGTGTGA GTTTGAGAAC ACTGTGCACA AGTGGATTTC CATCACCGAG GCCCTAGCTT | 1200 |
| | TCTTCCACTG TTGTCTGAAC CCCATCCTCT ATGCTTTCCT TGGAGCCAAA TTTAAAACCT | 1260 |
| | CTGCCCAGCA CGCACTCACC TCTGTGAGCA GAGGGTCCAG CCTCAAGATC CTCTCCAAAG | 1320 |
| | GAAAGCGAGG TGGACATTCA TCTGTTTCCA CTGAGTCTGA GTCTTCAAGT TTTCACTCCA | 1380 |
| | GCTAACACAG ATGTAAAAGA CTTTTTTTTA TACGATAAAT AACTTTTTTT TAAGTTACAC | 1440 |
| | ATTTTTCAGA TATAAAAGAC TGACCAATAT TGTACAGTTT TTATTGCTTG TTGGATTTTT | 1500 |
| | GTCTTGTGTT TCTTTAGTTT TTGTGAAGTT TAATTGACTT ATTTATATAA ATTTTTTTTG | 1560 |
| | TTTCATATTG ATGTGTGTCT AGGCAGGACC TGTGGCCAAG TTCTTAGTTG CTGTATGTCT | 1620 |
| | CGTGGTAGGA CTGTAGAAAA GGGAACTGAA CATTCCAGAG CGTGTAGTGA ATCACGTAAA | 1680 |
| | GCTAGAAATG ATCCCCAGCT GTTTATGCAT AGATAATCTC TCCATTCCCG TGGAACGTTT | 1740 |
| | TTCCTGTTCT TAAGACGTGA TTTTGCTGTA GAAGATGGCA CTTATAACCA AAGCCCAAAG | 1800 |
| | TGGTATAGAA ATGCTGGTTT TTCAGTTTTC AGGAGTGGGT TGATTTCAGC ACCTACAGTG | 1860 |
| | TACAGTCTTG TATTAAGTTG TTAATAAAAG TACATGTTAA ACTTA | 1905 |
| SEQ ID NO: 656 | CTCTCAACAT AAGACAGTGA CCAGTCTGGT GACTCACAGC CGGCACAGCC ATGAACTACC | 60 |
| CXCR5 variant 1 | CGCTAACGCT GGAAATGGAC CTCGAGAACC TGGAGGACCT GTTCTGGGAA CTGGACAGAT | 120 |
| domain | TGGACAACTA TAACGACACC TCCCTGGTGG AAAATCATCT CTGCCCTGCC ACAGAGGGGC | 180 |
| | CCCTCATGGC CTCCTTCAAG GCCGTGTTCG TGCCCGTGGC CTACAGCCTC ATCTTCCTCC | 240 |
| | TGGGCGTGAT CGGCAACGTC CTGGTGCTGG TGATCCTGGA GCGGCACCGG CAGACACGCA | 300 |
| | GTTCCACGGA GACCTTCCTG TTCCACCTGG CCGTGGCCGA CCTCCTGCTG GTCTTCATCT | 360 |
| | TGCCCTTTGC CGTGGCCGAG GGCTCTGTGG GCTGGGTCCT GGGGACCTTC CTCTGCAAAA | 420 |
| | CTGTGATTGC CCTGCACAAA GTCAACTTCT ACTGCAGCAG CCTGCTCCTG GCCTGCATCG | 480 |
| | CCGTGGACCG CTACCTGGCC ATTGTCCACG CCGTCCATGC CTACCGCCAC CGCCGCCTCC | 540 |
| | TCTCCATCCA CATCACCTGT GGGACCATCT GGCTGGTGGG CTTCCTCCTT GCCTTGCCAG | 600 |
| | AGATTCTCTT CGCCAAAGTC AGCCAAGGCC ATCACAACAA CTCCCTGCCA CGTTGCACCT | 660 |
| | TCTCCCAAGA GAACCAAGCA GAAACGCATG CCTGGTTCAC CTCCCGATTC CTCTACCATG | 720 |
| | TGGCGGGATT CCTGCTGCCC ATGCTGGTGA TGGGCTGGTG CTACGTGGGG GTAGTGCACA | 780 |
| | GGTTGCGCCA GGCCCAGCGG CGCCCTCAGC GGCAGAAGGC AGTCAGGGTG GCCATCCTGG | 840 |
| | TGACAAGCAT CTTCTTCCTC TGCTGGTCAC CCTACCACAT CGTCATCTTC CTGGACACCC | 900 |
| | TGGCGAGGCT GAAGGCCGTG GACAATACCT GCAAGCTGAA TGGCTCTCTC CCCGTGGCCA | 960 |
| | TCACCATGTG TGAGTTCCTG GGCCTGGCCC ACTGCTGCCT CAACCCCATG CTCTACACTT | 1020 |
| | TCGCCGGCGT GAAGTTCCGC AGTGACCTGT CGCGGCTCCT GACGAAGCTG GGCTGTACCG | 1080 |
| | GCCCTGCCTC CCTGTGCCAG CTCTTCCCTA GCTGGCGCAG GAGCAGTCTC TCTGAGTCAG | 1140 |
| | AGAATGCCAC CTCTCTCACC ACGTTCTAGG TCCCAGTGTC CCCTTTTATT GCTGCTTTTC | 1200 |
| | CTTGGGGCAG GCAGTGATGC TGGATGCTCC TTCCAACAGG AGCTGGGATC CTAAGGGCTC | 1260 |
| | ACCGTGGCTA AGAGTGTCCT AGGAGTATCC TCATTTGGGG TAGCTAGAGG AACCAACCCC | 1320 |
| | CATTTCTAGA ACATCCCTGC CAGCTCTTCT GCCGGCCCTG GGGCTAGGCT GGAGCCCAGG | 1380 |
| | GAGCGGAAAG CAGCTCAAAG GCACAGTGAA GGCTGTCCTT ACCCATCTGC ACCCCCCTGG | 1440 |
| | GCTGAGAGAA CCTCACGCAC CTCCCATCCT AATCATCCAA TGCTCAAGAA ACAACTTCTA | 1500 |
| | CTTCTGCCCT TGCCAACGGA GAGCGCCTGC CCCTCCCAGA ACACACTCCA TCAGCTTAGG | 1560 |
| | GGCTGCTGAC CTCCACAGCT TCCCCTCTCT CCTCCTGCCC ACCTGTCAAA CAAAGCCAGA | 1620 |
| | AGCTGAGCAC CAGGGGATGA GTGGAGGTTA AGGCTGAGGA AAGGCCAGCT GGCAGCAGAG | 1680 |
| | TGTGGCCTTC GGACAACTCA GTCCCTAAAA ACACAGACAT TCTGCCAGGC CCCCAAGCCT | 1740 |
| | GCAGTCATCT TGACCAAGCA GGAAGCTCAG ACTGGTTGAG TTCAGGTAGC TGCCCCTGGC | 1800 |
| | TCTGACCGAA ACAGCGCTGG GTCCACCCCA TGTCACCGGA TCCTGGGTGG TCTGCAGGCA | 1860 |
| | GGGCTGACTC TAGGTGCCCT TGGAGGCCAG CCAGTGACCT GAGGAAGCGT GAAGGCCGAG | 1920 |
| | AAGCAAGAAA GAAACCCGAC AGAGGGAAGA AAAGAGCTTT CTTCCCGAAC CCCAAGGAGG | 1980 |
| | GAGATGGATC AATCAAACCC GGCGGTCCCC TCCGCCAGGC GAGATGGGGT GGGGTGGAGA | 2040 |
| | ACTCCTAGGG TGGCTGGGTC CAGGGGATGG GAGGTTGTGG GCATTGATGG GGAAGGAGGC | 2100 |
| | TGGCTTGTCC CCTCCTCACT CCCTTCCCAT AAGCTATAGA CCCGAGGAAA CTCAGAGTCG | 2160 |
| | GAACGGAGAA AGGTGGACTG GAAGGGGCCC GTGGGAGTCA TCTCAACCAT CCCCTCCGTG | 2220 |
| | GCATCACCTT AGGCAGGGAA GTGTAAGAAA CACACTGAGG CAGGGAAGTC CCCAGGCCCC | 2280 |
| | AGGAAGCCGT GCCCTGCCCC CGTGAGGATG TCACTCAGAT GGAACCGCAG GAAGCTGCTC | 2340 |
| | CGTGCTTGTT TGCTCACCTG GGGTGTGGGA GGCCCGTCCG GCAGTTCTGG GTGCTCCCTA | 2400 |
| | CCACCTCCCC AGCCETTGAT CAGGTGGGGA GTCAGGGACC CCTGCCCTTG TCCCACTCAA | 2460 |
| | GCCAAGCAGC CAAGCTCCTT GGGAGGCCCC ACTGGGGAAA TAACAGCTGT GGCTCACGTG | 2520 |
| | AGAGTGTCTT CACGGCAGGA CAACGAGGAA GCCCTAAGAC GTCCCTTTTT TCTCTGAGTA | 2580 |
| | TCTCCTCGCA AGCTGGGTAA TCGATGGGGG AGTCTGAAGC AGATGCAAAG AGGCAAGAGG | 2640 |
| | CTGGATTTTG AATTTTCTTT TTAATAAAAA GGCACCTATA AAACAGGTCA ATACAGTACA | 2700 |
| | GGCAGCACAG AGACCCCCGG AACAAGCCTA AAAATTGTTT CAAAATAAAA ACCAAGAAGA | 2760 |
| | TGTCTTCACA TATTGTATTT ATATATTTAT ATTTATATAT ATATTTATAT AATGGTACAA | 2820 |
| | AATGGCTGGG GGTGTGGCCA TGGATGGAGG GAAGAGTAGG CTGGCCTGTG GCGTGGGTGG | 2880 |
| | GAGGAGAGGG GACGGAGAGG GCACTCGGCC CGCTGCAATC TGACCCCTCT CTCCTCAGGG | 2940 |
| | CAGGAAACAC AGAGTCAGAC AGTTTGGGGG GGTCTTGGGC CAGGGGTGGA GGGCTCAAGG | 3000 |
| | GCACAGGGCC CAGGCTGAGG CAGGGCGGGC AAAGCGCCTG GCAGGATGAA GGGCAAGTGG | 3060 |
| | CCCCCCAAAC ACAGAGGCCC TGGCCATGGA CCCTGGGAGG TGACCGGGGT GAGTCAGGGG | 3120 |
| | CCTGTTGTCA GCCCCAGAGG AAGCGCTGGA CCTGGCCGAT GGTGGGCCGA GAGGACAGCA | 3180 |
| | CCAGGCTGGG AGAAGTGGGG CGAGTTCCCT TTGTATTACA GCTGCCAGTG CAAGACCAGG | 3240 |
| | CCCTCCAGGC CAGGAAGGCT AGGGACGGGT CCTGGTAGAA GACACCCTGT CTAGAATGGC | 3300 |
| | CCTTGGTCCT GGAGGTGGGG CGCAAAAGGC CTCAGCCAGG GAACTGCCCT GCCACCTCCC | 3360 |
| | GAGGCAGGAA AGGAAGTGAG AAAAGGAGAA GTTTTTTTAC TCCTGGGGCC AAAGTAGGGG | 3420 |
| | GACAAACACC CAGTCGTATA TGGCTTCAGC TCTGACCAAA GGCGGATAGG GAGCTCTCCT | 3480 |
| | GGGTAGGAGC AGGGAGCCAA GGGGGAGGCA GTGGCTGTGC CTGGGTGGGC ACAGACAGAT | 3540 |
| | CTGGTACATG GCCCTGAGCC CTGGGCAGAG GGACAACCTT GCCGGTGAGT GGGCAGGCAG | 3600 |
| | AGAGGAGGCG GCAGGATGCT GTTTCCCCGA TTCCATCCTC AGGGAGTGGA GACTGGAGGG | 3660 |
| | GAGGTGCACT GACTCAGATG AACTGTTCTC CCCCTTCTTT GATAAGAAGT AGGTGGCAGC | 3720 |
| | AGCCTCTGGA AAAGTCAGGG CCCTGGAGGT TACCTGGCCC AGGGCTACTA CAGCCACAGG | 3780 |
| | CCCCAGTGGC ACCATGCCAC CCCTTCCATG GCTCCACTCA AGGGGGCCAC ACAGCCACCG | 3840 |
| | CCTCTTCCTC CTTTCCTTCA TCCCAAACTG GGACAAAAGA CTTCAAGTTC TGGCTAAGAT | 3900 |
| | GTAGCAGCAG CGGATGCCCG GGCATCCAAA GTGGAAAGCC AGGGCCCCGT GTCACCGGTG | 3960 |
| | TGGGCAAACA CACATGCACG TGCACACATG TTCTCCCTGA ATCACTCAGC AGCAGACAGG | 4020 |
| | CTGCCGCCCT GGGGGTCTCA GCCCTGCTAG GGCTCACCAG GTGGAAGCCT AGGTGGTCTG | 4080 |
| | ACCTCAGTTT AGGAGTGGGT CATTTACGTC ATCTTACCAT TTGGGGACGA GACAGGAATG | 4140 |
| | GTATCCCTTA GGGACCCAGA GACACTGCAA ACAGTGGGTG GCCATGTAGG GCTGCATGTC | 4200 |
| | CCTGGGTCCA GGGGAATGGA GGGAGCAATA ACTTGAAGAA GGGGGGAAGG GTTTCTTTTA | 4260 |
| | TCCTTTTTTT TTTGTGTGAC TTCTATCAAA ACA | 4293 |
| SEQ ID NO : 657 | CCACTCTAAG GAATGCGGTC CCTTTGACAG GCGAAAAACT GAAGTTGGAA AAGACAAAGT | 60 |
| CXCR5 variant 2 | GATTTGTTCA AAATTGAAAT TTGAAACTTG ACATTTGGTC AGTGGGCCCT ATGTAGGAAA | 120 |
| domain | AAACCTCCAA GAGAGCTAGG GTTCCTCTCA GAGAGGAAAG ACAGGTCCTT AGGTCCTCAC | 180 |
| | CCTCCCGTCT CCTTGCCCTT GCAGTTCTGG GAACTGGACA GATTGGACAA CTATAACGAC | 240 |
| | ACCTCCCTGG TGGAAAATCA TCTCTGCCCT GCCACAGAGG GGCCCCTCAT GGCCTCCTTC | 300 |
| | AAGGCCGTGT TCGTGCCCGT GGCCTACAGC CTCATCTTCC TCCTGGGCGT GATCGGCAAC | 360 |
| | GTCCTGGTGC TGGTGATCCT GGAGCGGCAC CGGCAGACAC GCAGTTCCAC GGAGACCTTC | 420 |
| | CTGTTCCACC TGGCCGTGGC CGACCTCCTG CTGGTCTTCA TCTTGCCCTT TGCCGTGGCC | 480 |
| | GAGGGCTCTG TGGGCTGGGT CCTGGGGACC TTCCTCTGCA AAACTGTGAT TGCCCTGCAC | 540 |
| | AAAGTCAACT TCTACTGCAG CAGCCTGCTC CTGGCCTGCA TCGCCGTGGA CCGCTACCTG | 600 |
| | GCCATTGTCC ACGCCGTCCA TGCCTACCGC CACCGCCGCC TCCTCTCCAT CCACATCACC | 660 |
| | TGTGGGACCA TCTGGCTGGT GGGCTTCCTC CTTGCCTTGC CAGAGATTCT CTTCGCCAAA | 720 |
| | GTCAGCCAAG GCCATCACAA CAACTCCCTG CCACGTTGCA CCTTCTCCCA AGAGAACCAA | 780 |
| | GCAGAAACGC ATGCCTGGTT CACCTCCCGA TTCCTCTACC ATGTGGCGGG ATTCCTGCTG | 840 |
| | CCCATGCTGG TGATGGGCTG GTGCTACGTG GGGGTAGTGC ACAGGTTGCG CCAGGCCCAG | 900 |
| | CGGCGCCCTC AGCGGCAGAA GGCAGTCAGG GTGGCCATCC TGGTGACAAG CATCTTCTTC | 960 |
| | CTCTGCTGGT CACCCTACCA CATCGTCATC TTCCTGGACA CCCTGGCGAG GCTGAAGGCC | 1020 |
| | GTGGACAATA CCTGCAAGCT GAATGGCTCT CTCCCCGTGG CCATCACCAT GTGTGAGTTC | 1080 |
| | CTGGGCCTGG CCCACTGCTG CCTCAACCCC ATGCTCTACA CTTTCGCCGG CGTGAAGTTC | 1140 |
| | CGCAGTGACC TGTCGCGGCT CCTGACGAAG CTGGGCTGTA CCGGCCCTGC CTCCCTGTGC | 1200 |
| | CAGCTCTTCC CTAGCTGGCG CAGGAGCAGT CTCTCTGAGT CAGAGAATGC CACCTCTCTC | 1260 |
| | ACCACGTTCT AGGTCCCAGT GTCCCCTTTT ATTGCTGCTT TTCCTTGGGG CAGGCAGTGA | 1320 |
| | TGCTGGATGC TCCTTCCAAC AGGAGCTGGG ATCCTAAGGG CTCACCGTGG CTAAGAGTGT | 1380 |
| | CCTAGGAGTA TCCTCATTTG GGGTAGCTAG AGGAACCAAC CCCCATTTCT AGAACATCCC | 1440 |
| | TGCCAGCTCT TCTGCCGGCC CTGGGGCTAG GCTGGAGCCC AGGGAGCGGA AAGCAGCTCA | 1500 |
| | AAGGCACAGT GAAGGCTGTC CTTACCCATC TGCACCCCCC TGGGCTGAGA GAACCTCACG | 1560 |
| | CACCTCCCAT CCTAATCATC CAATGCTCAA GAAACAACTT CTACTTCTGC CCTTGCCAAC | 1620 |
| | GGAGAGCGCC TGCCCCTCCC AGAACACACT CCATCAGCTT AGGGGCTGCT GACCTCCACA | 1680 |
| | GCTTCCCCTC TCTCCTCCTG CCCACCTGTC AAACAAAGCC AGAAGCTGAG CACCAGGGGA | 1740 |
| | TGAGTGGAGG TTAAGGCTGA GGAAAGGCCA GCTGGCAGCA GAGTGTGGCC TTCGGACAAC | 1800 |
| | TCAGTCCCTA AAAACACAGA CATTCTGCCA GGCCCCCAAG CCTGCAGTCA TCTTGACCAA | 1860 |
| | GCAGGAAGCT CAGACTGGTT GAGTTCAGGT AGCTGCCCCT GGCTCTGACC GAAACAGCGC | 1920 |
| | TGGGTCCACC CCATGTCACC GGATCCTGGG TGGTCTGCAG GCAGGGCTGA CTCTAGGTGC | 1980 |
| | CCTTGGAGGC CAGCCAGTGA CCTGAGGAAG CGTGAAGGCC GAGAAGCAAG AAAGAAACCC | 2040 |
| | GACAGAGGGA AGAAAAGAGC TTTCTTCCCG AACCCCAAGG AGGGAGATGG ATCAATCAAA | 2100 |
| | CCCGGCGGTC CCCTCCGCCA GGCGAGATGG GGTGGGGTGG AGAACTCCTA GGGTGGCTGG | 2160 |
| | GTCCAGGGGA TGGGAGGTTG TGGGCATTGA TGGGGAAGGA GGCTGGCTTG TCCCCTCCTC | 2220 |
| | ACTCCCTTCC CATAAGCTAT AGACCCGAGG AAACTCAGAG TCGGAACGGA GAAAGGTGGA | 2280 |
| | CTGGAAGGGG CCCGTGGGAG TCATCTCAAC CATCCCCTCC GTGGCATCAC CTTAGGCAGG | 2340 |
| | GAAGTGTAAG AAACACACTG AGGCAGGGAA GTCCCCAGGC CCCAGGAAGC CGTGCCCTGC | 2400 |
| | CCCCGTGAGG ATGTCACTCA GATGGAACCG CAGGAAGCTG CTCCGTGCTT GTTTGCTCAC | 2460 |
| | CTGGGGTGTG GGAGGCCCGT CCGGCAGTTC TGGGTGCTCC CTACCACCTC CCCAGCCTTT | 2520 |
| | GATCAGGTGG GGAGTCAGGG ACCCCTGCCC TTGTCCCACT CAAGCCAAGC AGCCAAGCTC | 2580 |
| | CTTGGGAGGC CCCACTGGGG AAATAACAGC TGTGGCTCAC GTGAGAGTGT CTTCACGGCA | 2640 |
| | GGACAACGAG GAAGCCCTAA GACGTCCCTT TTTTCTCTGA GTATCTCCTC GCAAGCTGGG | 2700 |
| | TAATCGATGG GGGAGTCTGA AGCAGATGCA AAGAGGCAAG AGGCTGGATT TTGAATTTTC | 2760 |
| | TTTTTAATAA AAAGGCACCT ATAAAACAGG TCAATACAGT ACAGGCAGCA CAGAGACCCC | 2820 |
| | CGGAACAAGC CTAAAAATTG TTTCAAAATA AAAACCAAGA AGATGTCTTC ACATATTGTA | 2880 |
| | AAAAAAAAAA AAAAAA | 2896 |
| SEQ ID NO: 658 | AGATCTGTTT GGTTCAGTTG CTGAGAAGCC TGACATACCA GGACTGCCTG AGACAAGCCA | 60 |
| CCR2 variant A | CAAGCTGAAC AGAGAAAGTG GATTGAACAA GGACGCATTT CCCCAGTACA TCCACAACAT | 120 |
| domain | GCTGTCCACA TCTCGTTCTC GGTTTATCAG AAATACCAAC GAGAGCGGTG AAGAAGTCAC | 180 |
| | CACCTTTTTT GATTATGATT ACGGTGCTCC CTGTCATAAA TTTGACGTGA AGCAAATTGG | 240 |
| | GGCCCAACTC CTGCCTCCGC TCTACTCGCT GGTGTTCATC TTTGGTTTTG TGGGCAACAT | 300 |
| | GCTGGTCGTC CTCATCTTAA TAAACTGCAA AAAGCTGAAG TGCTTGACTG ACATTTACCT | 360 |
| | GCTCAACCTG GCCATCTCTG ATCTGCTTTT TCTTATTACT CTCCCATTGT GGGCTCACTC | 420 |
| | TGCTGCAAAT GAGTGGGTCT TTGGGAATGC AATGTGCAAA TTATTCACAG GGCTGTATCA | 480 |
| | CATCGGTTAT TTTGGCGGAA TCTTCTTCAT CATCCTCCTG ACAATCGATA GATACCTGGC | 540 |
| | TATTGTCCAT GCTGTGTTTG CTTTAAAAGC CAGGACGGTC ACCTTTGGGG TGGTGACAAG | 600 |
| | TGTGATCACC TGGTTGGTGG CTGTGTTTGC TTCTGTCCCA GGAATCATCT TTACTAAATG | 660 |
| | CCAGAAAGAA GATTCTGTTT ATGTCTGTGG CCCTTATTTT CCACGAGGAT GGAATAATTT | 720 |
| | CCACACAATA ATGAGGAACA TTTTGGGGCT GGTCCTGCCG CTGCTCATCA TGGTCATCTG | 780 |
| | CTACTCGGGA ATCCTGAAAA CCCTGCTTCG GTGTCGAAAC GAGAAGAAGA GGCATAGGGC | 840 |
| | AGTGAGAGTC ATCTTCACCA TCATGATTGT TTACTTTCTC TTCTGGACTC CCTATAATAT | 900 |
| | TGTCATTCTC CTGAACACCT TCCAGGAATT CTTCGGCCTG AGTAACTGTG AAAGCACCAG | 960 |
| | TCAACTGGAC CAAGCCACGC AGGTGACAGA GACTCTTGGG ATGACTCACT GCTGCATCAA | 1020 |
| | TCCCATCATC TATGCCTTCG TTGGGGAGAA GTTCAGAAGC CTTTTTCACA TAGCTCTTGG | 1080 |
| | CTGTAGGATT GCCCCACTCC AAAAACCAGT GTGTGGAGGT CCAGGAGTGA GACCAGGAAA | 1140 |
| | GAATGTGAAA GTGACTACAC AAGGACTCCT CGATGGTCGT GGAAAAGGAA AGTCAATTGG | 1200 |
| | CAGAGCCCCT GAAGCCAGTC TTCAGGACAA AGAAGGAGCC TAGAGACAGA AATGACAGAT | 1260 |
| | CTCTGCTTTG GAAATCACAC GTCTGGCTTC ACAGATGTGT GATTCACAGT GTGAATCTTG | 1320 |
| | GTGTCTACGT TACCAGGCAG GAAGGCTGAG AGGAGAGAGA CTCCAGCTGG GTTGGAAAAC | 1380 |
| | AGTATTTTCC AAACTACCTT CCAGTTCCTC ATTTTTGAAT ACAGGCATAG AGTTCAGACT | 1440 |
| | TTTTTTAAAT AGTAAAAATA AAATTAAAGC TGAAAACTGC AACTTGTAAA TGTGGTAAAG | 1500 |
| | AGTTAGTTTG AGTTACTATC ATGTCAAACG TGAAAATGCT GTATTAGTCA CAGAGATAAT | 1560 |
| | TCTAGCTTTG AGCTTAAGAA TTTTGAGCAG GTGGTATGTT TGGGAGACTG CTGAGTCAAC | 1620 |
| | CCAATAGTTG TTGATTGGCA GGAGTTGGAA GTGTGTGATC TGTGGGCACA TTAGCCTATG | 1680 |
| | TGCATGCAGC ATCTAAGTAA TGATGTCGTT TGAATCACAG TATACGCTCC ATCGCTGTCA | 1740 |
| | TCTCAGCTGG ATCTCCATTC TCTCAGGCTT GCTGCCAAAA GCCTTTTGTG TTTTGTTTTG | 1800 |
| | TATCATTATG AAGTCATGCG TTTAATCACA TTCGAGTGTT TCAGTGCTTC GCAGATGTCC | 1860 |
| | TTGATGCTCA TATTGTTCCC TATTTTGCCA GTGGGAACTC CTAAATCAAG TTGGCTTCTA | 1920 |
| | ATCAAAGCTT TTAAACCCTA TTGGTAAAGA ATGGAAGGTG GAGAAGCTCC CTGAAGTAAG | 1980 |
| | CAAAGACTTT CCTCTTAGTC GAGCCAAGTT AAGAATGTTC TTATGTTGCC CAGTGTGTTT | 2040 |
| | CTGATCTGAT GCAAGCAAGA AACACTGGGC TTCTAGAACC AGGCAACTTG GGAACTAGAC | 2100 |
| | TCCCAAGCTG GACTATGGCT CTACTTTCAG GCCACATGGC TAAAGAAGGT TTCAGAAAGA | 2160 |
| | AGTGGGGACA GAGCAGAACT TTCACCTTCA TATATTTGTA TGATCCTAAT GAATGCATAA | 2220 |
| | AATGTTAAGT TGATGGTGAT GAAATGTAAA TACTGTTTTT AACAACTATG ATTTGGAAAA | 2280 |
| | TAAATCAATG CTATAACTAT GTTGATAAAA GATTTAAAAA CAA | 2323 |
| SEQ ID NO: 659 | AGATCTGTTT GGTTCAGTTG CTGAGAAGCC TGACATACCA GGACTGCCTG AGACAAGCCA | 60 |
| CCR2 variant B | CAAGCTGAAC AGAGAAAGTG GATTGAACAA GGACGCATTT CCCCAGTACA TCCACAACAT | 120 |
| domain | GCTGTCCACA TCTCGTTCTC GGTTTATCAG AAATACCAAC GAGAGCGGTG AAGAAGTCAC | 180 |
| | CACCTTTTTT GATTATGATT ACGGTGCTCC CTGTCATAAA TTTGACGTGA AGCAAATTGG | 240 |
| | GGCCCAACTC CTGCCTCCGC TCTACTCGCT GGTGTTCATC TTTGGTTTTG TGGGCAACAT | 300 |
| | GCTGGTCGTC CTCASCTTAA TAAACTGCAA AAAGCTGAAG TGCTTGACTG ACATTTACCT | 360 |
| | GCTCAACCTG GCCATCTCTG ATCTGCTTTT TCTTATTACT CTCCCATTGT GGGCTCACTC | 420 |
| | TGCTGCAAAT GAGTGGGTCT TTGGGAATGC AATGTGCAAA TTATTCACAG GGCTGTATCA | 480 |
| | CATCGGTTAT TTTGGCGGAA TCTTCTTCAT CATCCTCCTG ACAATCGATA GATACCTGGC | 540 |
| | TATTGTCCAT GCTGTGTTTG CTTTAAAAGC CAGGACGGTC ACCTTTGGGG TGGTGACAAG | 600 |
| | TGTGATCACC TGGTTGGTGG CTGTGTTTGC TTCTGTCCCA GGAATCATCT TTACTAAATG | 660 |
| | CCAGAAAGAA GATTCTGTTT ATGTCTGTGG CCCTTATTTT CCACGAGGAT GGAATAATTT | 720 |
| | CCACACAATA ATGAGGAACA TTTTGGGGCT GGTCCTGCCG CTGCTCATCA TGGTCATCTG | 780 |
| | CTACTCGGGA ATCCTGAAAA CCCTGCTTCG GTGTCGAAAC GAGAAGAAGA GGCATAGGGC | 840 |
| | AGTGAGAGTC ATCTTCACCA TCATGATTGT TTACTTTCTC TTCTGGACTC CCTATAATAT | 900 |
| | TGTCATTCTC CTGAACACCT TCCAGGAATT CTTCGGCCTG AGTAACTGTG AAAGCACCAG | 960 |
| | TCAACTGGAC CAAGCCACGC AGGTGACAGA GACTCTTGGG ATGACTCACT GCTGCATCAA | 1020 |
| | TCCCATCATC TATGCCTTCG TTGGGGAGAA GTTCAGAAGG TATCTCTCGG TGTTCTTCCG | 1080 |
| | AAAGCACATC ACCAAGCGCT TCTGCAAACA ATGTCCAGTT TTCTACAGGG AGACAGTGGA | 1140 |
| | TGGAGTGACT TCAACAAACA CGCCTTCCAC TGGGGAGCAG GAAGTCTCGG CTGGTTTATA | 1200 |
| | AAACGAGGAG CAGTTTGATT GTTGTTTATA AAGGGAGATA ACAATCTGTA TATAACAACA | 1260 |
| | AACTTCAAGG GTTTGTTGAA CAATAGAAAC CTGTAAAGCA GGTGCCCAGG AACCTCAGGG | 1320 |
| | CTGTGTGTAC TAATACAGAC TATGTCACCC AATGCATATC CAACATGTGC TCAGGGAATA | 1380 |
| | ATCCAGAAAA ACTGTGGGTA GAGACTTTGA CTCTCCAGAA AGCTCATCTC AGCTCCTGAA | 1440 |
| | AAATGCCTCA TTACCTTGTG CTAATCCTCT TTTTCTAGTC TTCATAATTT CTTCACTCAA | 1500 |
| | TCTCTGATTC TGTCAATGTC TTGAAATCAA GGGCCAGCTG GAGGTGAAGA AGAGAATGTG | 1560 |
| | ACAGGCACAG ATGAATGGGA GTGAGGGATA GTGGGGTCAG GGCTGAGAGG AGAAGGAGGG | 1620 |
| | AGACATGAGC ATGGCTGAGC CTGGACAAAG ACAAAGGTGA GCAAAGGGCT CACGCATTCA | 1680 |
| | GCCAGGAGAT GATACTGGTC CTTAGCCCCA TCTGCCACGT GTATTTAACC TTGAAGGGTT | 1740 |
| | CACCAGGTCA GGGAGAGTTT GGGAACTGCA ATAACCTGGG AGTTTTGGTG GAGTCCGATG | 1800 |
| | ATTCTCTTTT GCATAAGTGC ATGACATATT TTTGCTTTAT TACAGTTTAT CTATGGCACC | 1860 |
| | CATGCACCTT ACATTTGAAA TCTATGAAAT ATCATGCTCC ATTGTTCAGA TGCTTCTTAG | 1920 |
| | GCCACATCCC CCTGECTAAA AATTCAGAAA ATTTTTGTTT ATAAAAGATG CATTATCTAT | 1980 |
| | GATATGCTAA TATATGTATA TGCAATATAT ATAGGCTCTT GCTTGATCTC TCCAGGAGGT | 2040 |
| | AGTGATTATG AGAAGGGGGT GGAGAATGAT GAGTTCCTTC ACCAGGAGCA AAGGACGGGG | 2100 |
| | ATCGTGTGGA ACCACTGCAG AACTATTTCC GAAATCAACT AAGTGGAGAG AGCCAGGAAG | 2160 |
| | GCTGCATCAG AACCCAGTAA AGCTTCTTGT CTGGATCTGA GCTGGTTTGT TTTGTGCTTG | 2220 |
| | CTTTTCCCTG CCTTGCCACT CCCCTCACTC TTCTCTTTTC CCCACAGCCT TTTTCACATA | 2280 |
| | GCTCTTGGCT GTAGGATTGC CCCACTCCAA AAACCAGTGT GTGGAGGTCC AGGAGTGAGA | 2340 |
| | CCAGGAAAGA ATGTGAAAGT GACTACACAA GGACTCCTCG ATGGTCGTGG AAAAGGAAAG | 2400 |
| | TCAATTGGCA GAGCCCCTGA AGCCAGTCTT CAGGACAAAG AAGGAGCCTA GAGACAGAAA | 2460 |
| | TGACAGATCT CTGCTTTGGA AATCACACGT CTGGCTTCAC AGATGTGTGA TTCACAGTGT | 2520 |
| | GAATCTTGGT GTCTACGTTA CCAGGCAGGA AGGCTGAGAG GAGAGAGACT CCAGCTGGGT | 2580 |
| | TGGAAAACAG TATTTTCCAA ACTACCTTCC AGTTCCTCAT TTTTGAATAC AGGCATAGAG | 2640 |
| | TTCAGACTTT TTTTAAATAG TAAAAATAAA ATTAAAGCTG AAAACTGCAA CTTGTAAATG | 2700 |
| | TGGTAAAGAG TTAGTTTGAG TTACTATCAT GTCAAACGTG AAAATGCTGT ATTAGTCACA | 2760 |
| | GAGATAATTC TAGCTTTGAG CTTAAGAATT TTGAGCAGGT GGTATGTTTG GGAGACTGCT | 2820 |
| | GAGTCAACCC AATAGTTGTT GATTGGCAGG AGTTGGAAGT GTGTGATCTG TGGGCACATT | 2880 |
| | AGCCTATGTG CATGCAGCAT CTAAGTAATG ATGTCGTTTG AATCACAGTA TACGCTCCAT | 2940 |
| | CGCTGTCATC TCAGCTGGAT CTCCATTCTC TCAGGCTTGC TGCCAAAAGC CTTTTGTGTT | 3000 |
| | TTGTTTTGTA TCATTATGAA GTCATGCGTT TAATCACATT CGAGTGTTTC AGTGCTTCGC | 3060 |
| | AGATGTCCTT GATGCTCATA TTGTTCCCTA TTTTGCCAGT GGGAACTCCT AAATCAAGTT | 3120 |
| | GGCTTCTAAT CAAAGCTTTT AAACCCTATT GGTAAAGAAT GGAAGGTGGA GAAGCTCCCT | 3180 |
| | GAAGTAAGCA AAGACTTTCC TCTTAGTCGA GCCAAGTTAA GAATGTTCTT ATGTTGCCCA | 3240 |
| | GTGTGTTTCT GATCTGATGC AAGCAAGAAA CACTGGGCTT CTAGAACCAG GCAACTTGGG | 3300 |
| | AACTAGACTC CCAAGCTGGA CTATGGCTCT ACTTTCAGGC CACATGGCTA AAGAAGGTTT | 3360 |
| | CAGAAAGAAG TGGGGACAGA GCAGAACTTT CACCTTCATA TATTTGTATG ATCCTAATGA | 3420 |
| | ATGCATAAAA TGTTAAGTTG ATGGTGATGA AATGTAAATA CTGTTTTTAA CAACTATGAT | 3480 |
| | TTGGAAAATA AATCAATGCT ATAACTATGT TGATAAAAGA TTTAAAAACA A | 3531 |
| SEQ ID NO: 660 | GGCATTGCCT CACAGACCTT CCTCAGAGCC GCTTTCAGAA AAGCAAGCTG CTTCTGGTTG | 60 |
| CCR4 domain | GGCCCAGACC TGCCTTGAGG AGCCTGTAGA GTTAAAAAAT GAACCCCACG GATATAGCAG | 120 |
| | ACACCACCCT CGATGAAAGC ATATACAGCA ATTACTATCT GTATGAAAGT ATCCCCAAGC | 180 |
| | CTTGCACCAA AGAAGGCATC AAGGCATTTG GGGAGCTCTT CCTGCCCCCA CTGTATTCCT | 240 |
| | TGGTTTTTGT ATTTGGTCTG CTTGGAAATT CTGTGGTGGT TCTGGTCCTG TTCAAATACA | 300 |
| | AGCGGCTCAG GTCCATGACT GATGTGTACC TGCTCAACCT TGCCATCTCG GATCTGCTCT | 360 |
| | TCGTGTTTTC CCTCCCTTTT TGGGGCTACT ATGCAGCAGA CCAGTGGGTT TTTGGGCTAG | 420 |
| | GTCTGTGCAA GATGATTTCC TGGATGTACT TGGTGGGCTT TTACAGTGGC ATATTCTTTG | 480 |
| | TCATGCTCAT GAGCATTGAT AGATACCTGG CAATTGTGCA CGCGGTGTTT TCCTTGAGGG | 540 |
| | CAAGGACCTT GACTTATGGG GTCATCACCA GTTTGGCTAC ATGGTCAGTG GCTGTGTTCG | 600 |
| | CCTCCCTTCC TGGCETTCTG TTCAGCACTT GTTATACTGA GCGCAACCAT ACCTACTGCA | 660 |
| | AAACCAAGTA CTCTCTCAAC TCCACGACGT GGAAGGTTCT CAGCTCCCTG GAAATCAACA | 720 |
| | TTCTCGGATT GGTGATCCCC TTAGGGATCA TGCTGTTTTG CTACTCCATG ATCATCAGGA | 780 |
| | CCTTGCAGCA TTGTAAAAAT GAGAAGAAGA ACAAGGCGGT GAAGATGATC TTTGCCGTGG | 840 |
| | TGGTCCTCTT CCTTGGGTTC TGGACACCTT ACAACATAGT GCTCTTCCTA GAGACCCTGG | 900 |
| | TGGAGCTAGA AGTCCTTCAG GACTGCACCT TTGAAAGATA CTTGGACTAT GCCATCCAGG | 960 |
| | CCACAGAAAC TCTGGCTTTT GTTCACTGCT GCCTTAATCC CATCATCTAC TTTTTTCTGG | 1020 |
| | GGGAGAAATT TCGCAAGTAC ATCCTACAGC TCTTCAAAAC CTGCAGGGGC CTTTTTGTGC | 1080 |
| | TCTGCCAATA CTGTGGGCTC CTCCAAATTT ACTCTGCTGA CACCCCCAGC TCATCTTACA | 1140 |
| | CGCAGTCCAC CATGGATCAT GATCTCCATG ATGCTCTGTA GAAAAATGAA ATGGTGAAAT | 1200 |
| | GCAGAGTCAA TGAACTTTCC ACATTCAGAG CTTACTTAAA ATTGTATTTT AGTAAGAGAT | 1260 |
| | TCCTGAGCCA GTGTCAGGAG GAAGGCTTAC ACCCACAGTG GAAAGACAGC TTCTCATCCT | 1320 |
| | GCAGGCAGCT TTTTCTCTCC CACTAGACAA GTCCAGCCTG GCAAGGGTTC ACCTGGGCTG | 1380 |
| | AGGCATCCTT CCTCACACCA GGCTTGCCTG CAGGCATGAG TCAGTCTGAT GAGAACTCTG | 1440 |
| | AGCAGTGCTT GAATGAAGTT GTAGGTAATA TTGCAAGGCA AAGACTATTC CCTTCTAACC | 1500 |
| | TGAACTGATG GGTTTCTCCA GAGGGAATTG CAGAGTACTG GCTGATGGAG TAAATCGCTA | 1560 |
| | CCTTTTGCTG TGGCAAATGG GCCCTCTAAT TAATTTCTTG CTTTTGCGGA ACAATATAGA | 1620 |
| | TAACTGTTTT TCTAATAACA TATCTCAGGC AAAGTATATT CCATTGAGCC AGATGTATGA | 1680 |
| | AGAAACAATT AGCGAAGTGA TGAAACCAGA TCTCAATTAT TTATTGTAAA GGATTATCTG | 1740 |
| | TTAATTGAAA CCAAACTTTT TATACTGATA TAAGGGTAAG GATATGAAGA CATTAGCCAA | 1800 |
| | GGTCTGCTTT CCAAACGTGA ACTACAAGGC ATTCAAAATC CAAACATATT TATGAAAATT | 1860 |
| | CAAACACAGT TTCTCACTTG TTTGTGGACA TGTTTTGTTC TAATTTTAAC AGAGGAATAT | 1920 |
| | TAAAAAATTT TAAATAGGCT GGGCACGGTG GCCTGTAATC CCAGCACTGT GGGAGGCCAA | 1980 |
| | GGTGGGCGGA TCACCTGAGG TCAGGAGTTC GAGACCAGCC TGGCCAACAT GGAGAAACCC | 2040 |
| | TGTCTCTACT AAAAAATACA AAATTAGCCA GGTGTGGTGG CGCATGCCTG TAATCCCAGC | 2100 |
| | TACTCAGGAG GCTGAGGCTG GAGAATAACT TGAATCCGGG AGGTGGAGGT TGCGGTGAGC | 2160 |
| | CGAGATCGCG CCATTGTACT CCAACCTGGG CAAAAAGAGC GAAACTCTGT CTCAAAAAAA | 2220 |
| | AAAAAAAAAA ATTAAATAAT ACATAGGCCA AGAATACATT TATTTGAGGT CATTTACTTG | 2280 |
| | TTTTTTTTTT TTTTTTTTTT TTTGAGATGG AATCTTGCTC TGTCACCCAG TCTGGAGTGC | 2340 |
| | AGTGGCGCGA TCTCGGCTCA CTGCAAGCTC TGCCTCACGG GTTCGCACCA TTCTCCTGCC | 2400 |
| | TCAGCCTCCC AAGTAGGTGG GACTACAGGC ACCTGCCCCC ATGCCTGGCT AATTTTTTGT | 2460 |
| | ATTTTCAGTA GAGASGGGGT TTCACCATGT TAGCAAGGAT GGTTTTAATC TCCTGACCTC | 2520 |
| | GTGATCCACC CGCCTCGGCC TCCCAAAGTG CTGGGATTAC AGGTGTGAGC CATCACGCCC | 2580 |
| | GGCCACTTGT TTATTTTTTA TTTTATTTTA TTTTATTTTT GAGATGGAGT CTCACTCTGT | 2640 |
| | CACCCAAGCT GGAGTGCAGT GGCACTCGGT TCACTGCAAA GTCTGCCTCC CAGGTTCAAG | 2700 |
| | CGATTCTCCT GCCTCAGCTT CTCAAGCAGC TGGGATTAGA GGTGTGCACC ACTACGCCAG | 2760 |
| | GCTAATTTTT GTATTTTTAG TAGAGATGGG GTTTCACCAT ATTGGCCAGG CTAGTCTTGA | 2820 |
| | ACTCCTGACC TCAGGTGATC TGCCTGCTTC AGCCTCCCAA AGTGCTGGGA TTACAGGCGT | 2880 |
| | GAGCCACCTC GCCCAGCCAA GGTCTTTTAC TTGTTTATAA ACAGTCTCTT CATAATTAAA | 2940 |
| | ATTAAGGATT AATAAAGTAT GACAATACCT CCTTAATCAT TTTGAAGTGC CTGCTATCAA | 3000 |
| | TTGAAATAAA AACAATCAAC TAAAA | 3025 |
| SEQ ID NO: 661 | AGCACACCAC CCAGTGTATG GGTGAAGGAG GCAGCAGTGT GGCCGGAGAG GAGAGCTGGG | 60 |
| CCR6 variant 1 | CTGGGAGCAC AGGAAGGTCC CCAGGACTCT GTGGTCATCA GTAAGAGAGG GCCCACGTGT | 120 |
| domain | ATATGCTGGT GAACAGAAAT GTCAACCTTT TCAAAGTCTG ACATTTAAGA GAAAAAACTG | 180 |
| | TGGCTGTTGG TTTGEGGAAC AGACAGCTCC TTCTTTATTG AGTCACCTCT ACTTTCCTGC | 240 |
| | TACCGCTGCC TGTGAGCTGA AGGGGCTGAA CCATACACTC CTTTTTCTAC AACCAGCTTG | 300 |
| | CATTTTTTCT GCCCACAATG AGCGGGGAAT CAATGAATTT CAGCGATGTT TTCGACTCCA | 360 |
| | GTGAAGATTA TTTTGTGTCA GTCAATACTT CATATTACTC AGTTGATTCT GAGATGTTAC | 420 |
| | TGTGCTCCTT GCAGGAGGTC AGGCAGTTCT CCAGGCTATT TGTACCGATT GCCTACTCCT | 480 |
| | TGATCTGTGT CTTTGGCCTC CTGGGGAATA TTCTGGTGGT GATCACCTTT GCTTTTTATA | 540 |
| | AGAAGGCCAG GTCTATGACA GACGTCTATC TCTTGAACAT GGCCATTGCA GACATCCTCT | 600 |
| | TTGTTCTTAC TCTCCCATTC TGGGCAGTGA GTCATGCCAC CGGTGCGTGG GTTTTCAGCA | 660 |
| | ATGCCACGTG CAAGETGCTA AAAGGCATCT ATGCCATCAA CTTTAACTGC GGGATGCTGC | 720 |
| | TCCTGACTTG CATTAGCATG GACCGGTACA TCGCCATTGT ACAGGCGACT AAGTCATTCC | 780 |
| | GGCTCCGATC CAGAACACTA CCGCGCAGCA AAATCATCTG CCTTGTTGTG TGGGGGCTGT | 840 |
| | CAGTCATCAT CTCCAGCTCA ACTTTTGTCT TCAACCAAAA ATACAACACC CAAGGCAGCG | 900 |
| | ATGTCTGTGA ACCCAAGTAC CAGACTGTCT CGGAGCCCAT CAGGTGGAAG CTGCTGATGT | 960 |
| | TGGGGCTTGA GCTACTCTTT GGTTTCTTTA TCCCTTTGAT GTTCATGATA TTTTGTTACA | 1020 |
| | CGTTCATTGT CAAAACCTTG GTGCAAGCTC AGAATTCTAA AAGGCACAAA GCCATCCGTG | 1080 |
| | TAATCATAGC TGTGGTGCTT GTGTTTCTGG CTTGTCAGAT TCCTCATAAC ATGGTCCTGC | 1140 |
| | TTGTGACGGC TGCAAATTTG GGTAAAATGA ACCGATCCTG CCAGAGCGAA AAGCTAATTG | 1200 |
| | GCTATACGAA AACTGTCACA GAAGTCCTGG CTTTCCTGCA CTGCTGCCTG AACCCTGTGC | 1260 |
| | TCTACGCTTT TATTGGGCAG AAGTTCAGAA ACTACTTTCT GAAGATCTTG AAGGACCTGT | 1320 |
| | GGTGTGTGAG AAGGAAGTAC AAGTCCTCAG GCTTCTCCTG TGCCGGGAGG TACTCAGAAA | 1380 |
| | ACATTTCTCG GCAGACCAGT GAGACCGCAG ATAACGACAA TGCGTCGTCC TTCACTATGT | 1440 |
| | GATAGAAAGC TGAGTCTCCC TAAGGCATGT GTGAAACATA CTCATAGATG TTATGCAAAA | 1500 |
| | AAAAGTCTAT GGCCAGGTAT GCATGGAAAA TGTGGGAATT AAGCAAAATC AAGCAAGCCT | 1560 |
| | CTCTCCTGCG GGACETAACG TGCTCATGGG CTGTGTGATC TCTTCAGGGT GGGGTGGTCT | 1620 |
| | CTGATAGGTA GCATTTTCCA GCACTTTGCA AGGAATGTTT TGTAGCTCTA GGGTATATAT | 1680 |
| | CCGCCTGGCA TTTCACAAAA CAGCCTTTGG GAAATGCTGA ATTAAAGTGA ATTGTTGACA | 1740 |
| | AATGTAAACA TTTTCAGAAA TATTCATGAA GCGGTCACAG ATCACAGTGT CTTTTGGTTA | 1800 |
| | CAGCACAAAA TGATGGCAGT GGTTTGAAAA ACTAAAACAG AAAAAAAAAT GGAAGCCAAC | 1860 |
| | ACATCACTCA TTTTAGGCAA ATGTTTAAAC ATTTTTATCT ATCAGAATGT TTATTGTTGC | 1920 |
| | TGGTTATAAG CAGCAGGATT GGCCGGCTAG TGTTTCCTCT CATTTCCCTT TGATACAGTC | 1980 |
| | AACAAGCCTG ACCCTGTAAA ATGGAGGTGG AAAGACAAGC TCAAGTGTTC ACAACCTGGA | 2040 |
| | AGTGCTTCGG GAAGAAGGGG ACAATGGCAG AACAGGTGTT GGTGACAATT GTCACCAATT | 2100 |
| | GGATAAAGCA GCTCAGGTTG TAGTGGGCCA TTAGGAAACT GTCGGTTTGC TTTGATTTCC | 2160 |
| | CTGGGAGCTG TTCTCTGTCG TGAGTGTCTC TTGTCTAAAC GTCCATTAAG CTGAGAGTGC | 2220 |
| | TATGAAGACA GGATCTAGAA TAATCTTGCT CACAGCTGTG CTCTGAGTGC CTAGCGGAGT | 2280 |
| | TCCAGCAAAC AAAASGGACT CAAGAGAGAT TTGATTAATG AATCGTAATG AAGTTGGGGT | 2340 |
| | TTATTGTACA GTTTAAAATG TTAGATGTTT TTAATTTTTT AAATAAATGG AATACTTTTT | 2400 |
| | TTTTTTTTTT AAAGAAAGCA ACTTTACTGA GACAATGTAG AAAGAAGTTT TGTTCCGTTT | 2460 |
| | CTTTAATGTG GTTGAAGAGC AATGTGTGGC TGAAGACTTT TGTTATGAGG AGCTGCAGAT | 2520 |
| | TAGCTAGGGG ACAGCTGGAA TTATGCTGGC TTCTGATAAT TATTTTAAAG GGGTCTGAAA | 2580 |
| | TTTGTGATGG AATCAGATTT TAACAGCTCT CTTCAATGAC ATAGAAAGTT CATGGAACTC | 2640 |
| | ATGTTTTTAA AGGGCTATGT AAATATATGA ACATTAGAAA AATAGCAACT TGTGTTACAA | 2700 |
| | AAATACAAAC ACATGTTAGG AAGGTACTGT CATGGGCTAG GCATGGTGGC TCACACCTGT | 2760 |
| | AATCCCAGCA TTTTGGGAAG CTAAGATGGG TGGATCACTT GAGGTCAGGA GTTTGAGACC | 2820 |
| | AGCCTGGCCA ACATGGCGAA ACCCCTCTCT ACTAAAAATA CAAAAATTTG CCAGGCGTGG | 2880 |
| | TGGCGGGTGC CTGTAATCCC AGCTACTTGG GAGGCTGAGG CAAGAGAATC GCTTGAACCC | 2940 |
| | AGGAGGCAGA GGTTGCAGTG AGCCGAGATC GTGCCATTGC ACTCCAGCCT GGGTGACAAA | 3000 |
| | GCGAGACTCC ATCTCAAAAA AAAAAAAAAA AAAAAAGGAA AGAACTGTCA TGTAAACATA | 3060 |
| | CCAACATGTT TAAACCTGAC AATGGTGTTA TTTGAAACTT TATATTGTTC TTGTAAGCTT | 3120 |
| | TAACTATATC TCTCTTTAAA ATGCAAAATA ATGTCTTAAG ATTCAAAGTC TGTATTTTTA | 3180 |
| | AAGCATGGCT TTGGCTTTGC AAAATAAAAA ATGTGTTTTG TACATGAA | 3228 |
| SEQ ID NO: 662 | GCTTTTTTCT TAATGACTGC TAGAAGCTGC ATCTTATTGA CAGATGGTCA TCACATTGGT | 60 |
| CCR6 variant 2 | GAGCTGGAGT CATCAGATTG TGGGGCCCGG AGTGAGGCTG AAGGGAGTGG ATCAGAGCAC | 120 |
| domain | TGCCTGAGAG TCACCTCTAC TTTCCTGCTA CCGCTGCCTG TGAGCTGAAG GGGCTGAACC | 180 |
| | ATACACTCCT TTTTCTACAA CCAGCTTGCA TTTTTTCTGC CCACAATGAG CGGGGAATCA | 240 |
| | ATGAATTTCA GCGATGTTTT CGACTCCAGT GAAGATTATT TTGTGTCAGT CAATACTTCA | 300 |
| | TATTACTCAG TTGASTCTGA GATGTTACTG TGCTCCTTGC AGGAGGTCAG GCAGTTCTCC | 360 |
| | AGGCTATTTG TACCGATTGC CTACTCCTTG ATCTGTGTCT TTGGCCTCCT GGGGAATATT | 420 |
| | CTGGTGGTGA TCACCTTTGC TTTTTATAAG AAGGCCAGGT CTATGACAGA CGTCTATCTC | 480 |
| | TTGAACATGG CCATTGCAGA CATCCTCTTT GTTCTTACTC TCCCATTCTG GGCAGTGAGT | 540 |
| | CATGCCACCG GTGCGTGGGT TTTCAGCAAT GCCACGTGCA AGTTGCTAAA AGGCATCTAT | 600 |
| | GCCATCAACT TTAACTGCGG GATGCTGCTC CTGACTTGCA TTAGCATGGA CCGGTACATC | 660 |
| | GCCATTGTAC AGGCGACTAA GTCATTCCGG CTCCGATCCA GAACACTACC GCGCAGCAAA | 720 |
| | ATCATCTGCC TTGTTGTGTG GGGGCTGTCA GTCATCATCT CCAGCTCAAC TTTTGTCTTC | 780 |
| | AACCAAAAAT ACAACACCCA AGGCAGCGAT GTCTGTGAAC CCAAGTACCA GACTGTCTCG | 840 |
| | GAGCCCATCA GGTGGAAGCT GCTGATGTTG GGGCTTGAGC TACTCTTTGG TTTCTTTATC | 900 |
| | CCTTTGATGT TCATGATATT TTGTTACACG TTCATTGTCA AAACCTTGGT GCAAGCTCAG | 960 |
| | AATTCTAAAA GGCACAAAGC CATCCGTGTA ATCATAGCTG TGGTGCTTGT GTTTCTGGCT | 1020 |
| | TGTCAGATTC CTCATAACAT GGTCCTGCTT GTGACGGCTG CAAATTTGGG TAAAATGAAC | 1080 |
| | CGATCCTGCC AGAGCGAAAA GCTAATTGGC TATACGAAAA CTGTCACAGA AGTCCTGGCT | 1140 |
| | TTCCTGCACT GCTGCCTGAA CCCTGTGCTC TACGCTTTTA TTGGGCAGAA GTTCAGAAAC | 1200 |
| | TACTTTCTGA AGATCTTGAA GGACCTGTGG TGTGTGAGAA GGAAGTACAA GTCCTCAGGC | 1260 |
| | TTCTCCTGTG CCGGGAGGTA CTCAGAAAAC ATTTCTCGGC AGACCAGTGA GACCGCAGAT | 1320 |
| | AACGACAATG CGTCGTCCTT CACTATGTGA TAGAAAGCTG AGTCTCCCTA AGGCATGTGT | 1380 |
| | GAAACATACT CATAGATGTT ATGCAAAAAA AAGTCTATGG CCAGGTATGC ATGGAAAATG | 1440 |
| | TGGGAATTAA GCAAAATCAA GCAAGCCTCT CTCCTGCGGG ACTTAACGTG CTCATGGGCT | 1500 |
| | GTGTGATCTC TTCAGGGTGG GGTGGTCTCT GATAGGTAGC ATTTTCCAGC ACTTTGCAAG | 1560 |
| | GAATGTTTTG TAGCTCTAGG GTATATATCC GCCTGGCATT TCACAAAACA GCCTTTGGGA | 1620 |
| | AATGCTGAAT TAAAGTGAAT TGTTGACAAA TGTAAACATT TTCAGAAATA TTCATGAAGC | 1680 |
| | GGTCACAGAT CACAGTGTCT TTTGGTTACA GCACAAAATG ATGGCAGTGG TTTGAAAAAC | 1740 |
| | TAAAACAGAA AAAAAAATGG AAGCCAACAC ATCACTCATT TTAGGCAAAT GTTTAAACAT | 1800 |
| | TTTTATCTAT CAGAATGTTT ATTGTTGCTG GTTATAAGCA GCAGGATTGG CCGGCTAGTG | 1860 |
| | TTTCCTCTCA TTTCCCTTTG ATACAGTCAA CAAGCCTGAC CCTGTAAAAT GGAGGTGGAA | 1920 |
| | AGACAAGCTC AAGTGTTCAC AACCTGGAAG TGCTTCGGGA AGAAGGGGAC AATGGCAGAA | 1980 |
| | CAGGTGTTGG TGACAATTGT CACCAATTGG ATAAAGCAGC TCAGGTTGTA GTGGGCCATT | 2040 |
| | AGGAAACTGT CGGTTTGCTT TGATTTCCCT GGGAGCTGTT CTCTGTCGTG AGTGTCTCTT | 2100 |
| | GTCTAAACGT CCATTAAGCT GAGAGTGCTA TGAAGACAGG ATCTAGAATA ATCTTGCTCA | 2160 |
| | CAGCTGTGCT CTGAGTGCCT AGCGGAGTTC CAGCAAACAA AATGGACTCA AGAGAGATTT | 2220 |
| | GATTAATGAA TCGTAATGAA GTTGGGGTTT ATTGTACAGT TTAAAATGTT AGATGTTTTT | 2280 |
| | AATTTTTTAA ATAAATGGAA TACTTTTTTT TTTTTTTTAA AGAAAGCAAC TTTACTGAGA | 2340 |
| | CAATGTAGAA AGAAGTTTTG TTCCGTTTCT TTAATGTGGT TGAAGAGCAA TGTGTGGCTG | 2400 |
| | AAGACTTTTG TTATGAGGAG CTGCAGATTA GCTAGGGGAC AGCTGGAATT ATGCTGGCTT | 2460 |
| | CTGATAATTA TTTTAAAGGG GTCTGAAATT TGTGATGGAA TCAGATTTTA ACAGCTCTCT | 2520 |
| | TCAATGACAT AGAAAGTTCA TGGAACTCAT GTTTTTAAAG GGCTATGTAA ATATATGAAC | 2580 |
| | ATTAGAAAAA TAGCAACTTG TGTTACAAAA ATACAAACAC ATGTTAGGAA GGTACTGTCA | 2640 |
| | TGGGCTAGGC ATGGTGGCTC ACACCTGTAA TCCCAGCATT TTGGGAAGCT AAGATGGGTG | 2700 |
| | GATCACTTGA GGTCAGGAGT TTGAGACCAG CCTGGCCAAC ATGGCGAAAC CCCTCTCTAC | 2760 |
| | TAAAAATACA AAAATTTGCC AGGCGTGGTG GCGGGTGCCT GTAATCCCAG CTACTTGGGA | 2820 |
| | GGCTGAGGCA AGAGAATCGC TTGAACCCAG GAGGCAGAGG TTGCAGTGAG CCGAGATCGT | 2880 |
| | GCCATTGCAC TCCAGCCTGG GTGACAAAGC GAGACTCCAT CTCAAAAAAA AAAAAAAAAA | 2940 |
| | AAAAGGAAAG AACTGTCATG TAAACATACC AACATGTTTA AACCTGACAA TGGTGTTATT | 3000 |
| | TGAAACTTTA TATTGTTCTT GTAAGCTTTA ACTATATCTC TCTTTAAAAT GCAAAATAAT | 3060 |
| | GTCTTAAGAT TCAAAGTCTG TATTTTTAAA GCATGGCTTT GGCTTTGCAA AATAAAAAAT | 3120 |
| | GTGTTTTGTA CATGAA | 3136 |
| SEQ ID NO: 663 | AGACAGGGGT AGTGCGAGGC CGGGCACAGC CTTCCTGTGT GGTTTTACCG CCCAGAGAGC | 60 |
| CCR7 variant 1 | GTCATGGACC TGGGGAAACC AATGAAAAGC GTGCTGGTGG TGGCTCTCCT TGTCATTTTC | 120 |
| domain | CAGGTATGCC TGTGTCAAGA TGAGGTCACG GACGATTACA TCGGAGACAA CACCACAGTG | 180 |
| | GACTACACTT TGTTCGAGTC TTTGTGCTCC AAGAAGGACG TGCGGAACTT TAAAGCCTGG | 240 |
| | TTCCTCCCTA TCATGTACTC CATCATTTGT TTCGTGGGCC TACTGGGCAA TGGGCTGGTC | 300 |
| | GTGTTGACCT ATATCTATTT CAAGAGGCTC AAGACCATGA CCGATACCTA CCTGCTCAAC | 360 |
| | CTGGCGGTGG CAGACATCCT CTTCCTCCTG ACCCTTCCCT TCTGGGCCTA CAGCGCGGCC | 420 |
| | AAGTCCTGGG TCTTCGGTGT CCACTTTTGC AAGCTCATCT TTGCCATCTA CAAGATGAGC | 480 |
| | TTCTTCAGTG GCATGCTCCT ACTTCTTTGC ATCAGCATTG ACCGCTACGT GGCCATCGTC | 540 |
| | CAGGCTGTCT CAGCTCACCG CCACCGTGCC CGCGTCCTTC TCATCAGCAA GCTGTCCTGT | 600 |
| | GTGGGCATCT GGATACTAGC CACAGTGCTC TCCATCCCAG AGCTCCTGTA CAGTGACCTC | 660 |
| | CAGAGGAGCA GCAGTGAGCA AGCGATGCGA TGCTCTCTCA TCACAGAGCA TGTGGAGGCC | 720 |
| | TTTATCACCA TCCAGGTGGC CCAGATGGTG ATCGGCTTTC TGGTCCCCCT GCTGGCCATG | 780 |
| | AGCTTCTGTT ACCTTGTCAT CATCCGCACC CTGCTCCAGG CACGCAACTT TGAGCGCAAC | 840 |
| | AAGGCCATCA AGGTGATCAT CGCTGTGGTC GTGGTCTTCA TAGTCTTCCA GCTGCCCTAC | 900 |
| | AATGGGGTGG TCCTGGCCCA GACGGTGGCC AACTTCAACA TCACCAGTAG CACCTGTGAG | 960 |
| | CTCAGTAAGC AACTCAACAT CGCCTACGAC GTCACCTACA GCCTGGCCTG CGTCCGCTGC | 1020 |
| | TGCGTCAACC CTTTCTTGTA CGCCTTCATC GGCGTCAAGT TCCGCAACGA TCTCTTCAAG | 1080 |
| | CTCTTCAAGG ACCTGGGCTG CCTCAGCCAG GAGCAGCTCC GGCAGTGGTC TTCCTGTCGG | 1140 |
| | CACATCCGGC GCTCCTCCAT GAGTGTGGAG GCCGAGACCA CCACCACCTT CTCCCCATAG | 1200 |
| | GCGACTCTTC TGCCTGGACT AGAGGGACCT CTCCCAGGGT CCCTGGGGTG GGGATAGGGA | 1260 |
| | GCAGATGCAA TGACTCAGGA CATCCCCCCG CCAAAAGCTG CTCAGGGAAA AGCAGCTCTC | 1320 |
| | CCCTCAGAGT GCAAGCCCCT GCTCCAGAAG ATAGCTTCAC CCCAATCCCA GCTACCTCAA | 1380 |
| | CCAATGCCAA AAAAAGACAG GGCTGATAAG CTAACACCAG ACAGACAACA CTGGGAAACA | 1440 |
| | GAGGCTATTG TCCCCTAAAC CAAAAACTGA AAGTGAAAGT CCAGAAACTG TTCCCACCTG | 1500 |
| | CTGGAGTGAA GGGGCCAAGG AGGGTGAGTG CAAGGGGCGT GGGAGTGGCC TGAAGAGTCC | 1560 |
| | TCTGAATGAA CCTTCTGGCC TCCCACAGAC TCAAATGCTC AGACCAGCTC TTCCGAAAAC | 1620 |
| | CAGGCCTTAT CTCCAAGACC AGAGATAGTG GGGAGACTTC TTGGCTTGGT GAGGAAAAGC | 1680 |
| | GGACATCAGC TGGTCAAACA AACTCTCTGA ACCCCTCCCT CCATCGTTTT CTTCACTGTC | 1740 |
| | CTCCAAGCCA GCGGGAATGG CAGCTGCCAC GCCGCCCTAA AAGCACACTC ATCCCCTCAC | 1800 |
| | TTGCCGCGTC GCCCTCCCAG GCTCTCAACA GGGGAGAGTG TGGTGTTTCC TGCAGGCCAG | 1860 |
| | GCCAGCTGCC TCCGCGTGAT CAAAGCCACA CTCTGGGCTC CAGAGTGGGG ATGACATGCA | 1920 |
| | CTCAGCTCTT GGCTCCACTG GGATGGGAGG AGAGGACAAG GGAAATGTCA GGGGCGGGGA | 1980 |
| | GGGTGACAGT GGCCGCCCAA GGCCCACGAG CTTGTTCTTT GTTCTTTGTC ACAGGGACTG | 2040 |
| | AAAACCTCTC CTCATGTTCT GCTTTCGATT CGTTAAGAGA GCAACATTTT ACCCACACAC | 2100 |
| | AGATAAAGTT TTCCCTTGAG GAAACAACAG CTTTAAAAGA AAAAGAAAAA AAAAGTCTTT | 2160 |
| | GGTAAATGGC AAA | 2173 |
| SEQ ID NO: 664 | AGACAGGGGT AGTGCGAGGC CGGGCACAGC CTTCCTGTGT GGTTTTACCG CCCAGAGAGC | 60 |
| CCR7 variant 2 | GTCATGGACC TGGGTATGCC TGTGTCAAGA TGAGGTCACG GACGATTACA TCGGAGACAA | 120 |
| domain | CACCACAGTG GACTACACTT TGTTCGAGTC TTTGTGCTCC AAGAAGGACG TGCGGAACTT | 180 |
| | TAAAGCCTGG TTCCCCCCTA TCATGTACTC CATCATTTGT TTCGTGGGCC TACTGGGCAA | 240 |
| | TGGGCTGGTC GTGTTGACCT ATATCTATTT CAAGAGGCTC AAGACCATGA CCGATACCTA | 300 |
| | CCTGCTCAAC CTGGCGGTGG CAGACATCCT CTTCCTCCTG ACCCTTCCCT TCTGGGCCTA | 360 |
| | CAGCGCGGCC AAGTCCTGGG TCTTCGGTGT CCACTTTTGC AAGCTCATCT TTGCCATCTA | 420 |
| | CAAGATGAGC TTCTTCAGTG GCATGCTCCT ACTTCTTTGC ATCAGCATTG ACCGCTACGT | 480 |
| | GGCCATCGTC CAGGCTGTCT CAGCTCACCG CCACCGTGCC CGCGTCCTTC TCATCAGCAA | 540 |
| | GCTGTCCTGT GTGGGCATCT GGATACTAGC CACAGTGCTC TCCATCCCAG AGCTCCTGTA | 600 |
| | CAGTGACCTC CAGAGGAGCA GCAGTGAGCA AGCGATGCGA TGCTCTCTCA TCACAGAGCA | 660 |
| | TGTGGAGGCC TTTATCACCA TCCAGGTGGC CCAGATGGTG ATCGGCTTTC TGGTCCCCCT | 720 |
| | GCTGGCCATG AGCTTCTGTT ACCTTGTCAT CATCCGCACC CTGCTCCAGG CACGCAACTT | 780 |
| | TGAGCGCAAC AAGGCCATCA AGGTGATCAT CGCTGTGGTC GTGGTCTTCA TAGTCTTCCA | 840 |
| | GCTGCCCTAC AATGGGGTGG TCCTGGCCCA GACGGTGGCC AACTTCAACA TCACCAGTAG | 900 |
| | CACCTGTGAG CTCAGTAAGC AACTCAACAT CGCCTACGAC GTCACCTACA GCCTGGCCTG | 960 |
| | CGTCCGCTGC TGCGTCAACC CTTTCTTGTA CGCCTTCATC GGCGTCAAGT TCCGCAACGA | 1020 |
| | TCTCTTCAAG CTCTTCAAGG ACCTGGGCTG CCTCAGCCAG GAGCAGCTCC GGCAGTGGTC | 1080 |
| | TTCCTGTCGG CACATCCGGC GCTCCTCCAT GAGTGTGGAG GCCGAGACCA CCACCACCTT | 1140 |
| | CTCCCCATAG GCGACTCTTC TGCCTGGACT AGAGGGACCT CTCCCAGGGT CCCTGGGGTG | 1200 |
| | GGGATAGGGA GCAGATGCAA TGACTCAGGA CATCCCCCCG CCAAAAGCTG CTCAGGGAAA | 1260 |
| | AGCAGCTCTC CCCTCAGAGT GCAAGCCCCT GCTCCAGAAG ATAGCTTCAC CCCAATCCCA | 1320 |
| | GCTACCTCAA CCAATGCCAA AAAAAGACAG GGCTGATAAG CTAACACCAG ACAGACAACA | 1380 |
| | CTGGGAAACA GAGGCTATTG TCCCCTAAAC CAAAAACTGA AAGTGAAAGT CCAGAAACTG | 1440 |
| | TTCCCACCTG CTGGAGTGAA GGGGCCAAGG AGGGTGAGTG CAAGGGGCGT GGGAGTGGCC | 1500 |
| | TGAAGAGTCC TCTGAATGAA CCTTCTGGCC TCCCACAGAC TCAAATGCTC AGACCAGCTC | 1560 |
| | TTCCGAAAAC CAGGCCTTAT CTCCAAGACC AGAGATAGTG GGGAGACTTC TTGGCTTGGT | 1620 |
| | GAGGAAAAGC GGACATCAGC TGGTCAAACA AACTCTCTGA ACCCCTCCCT CCATCGTTTT | 1680 |
| | CTTCACTGTC CTCCAAGCCA GCGGGAATGG CAGCTGCCAC GCCGCCCTAA AAGCACACTC | 1740 |
| | ATCCCCTCAC TTGCCGCGTC GCCCTCCCAG GCTCTCAACA GGGGAGAGTG TGGTGTTTCC | 1800 |
| | TGCAGGCCAG GCCAGCTGCC TCCGCGTGAT CAAAGCCACA CTCTGGGCTC CAGAGTGGGG | 1860 |
| | ATGACATGCA CTCAGCTCTT GGCTCCACTG GGATGGGAGG AGAGGACAAG GGAAATGTCA | 1920 |
| | GGGGGGGGGA GGGTGACAGT GGCCGCCCAA GGCCCACGAG CTTGTTCTTT GTTCTTTGTC | 1980 |
| | ACAGGGACTG AAAACCTCTC CTCATGTTCT GCTTTCGATT CGTTAAGAGA GCAACATTTT | 2040 |
| | ACCCACACAC AGATAAAGTT TTCCCTTGAG GAAACAACAG CTTTAAAAGA AAAAGAAAAA | 2100 |
| | AAAAGTCTTT GGTAAATGGC AAA | 2123 |
| SEQ ID NO: 665 | CTCTAGATGA GTCAGTGGAG GGCGGGTGGA GCGTTGAACC GTGAAGAGTG TGGTTGGGCG | 60 |
| CCR7 variant 3 | TAAACGTGGA CTTAAACTCA GGAGCTAAGG GGTAATTCAG TGAAAAAGGG GAATGAGCGG | 120 |
| domain | TGGGGAGCTC TGTTGCAACA GGGTCCAATC GCAGCAGGAC TACAAATGCC CGAGCGCAGG | 180 |
| | CTGGGAACGA GGGGACAGCG GCTGCCTGTC CCCAGAATAG AAAATGCAGC TAGGAAGCCC | 240 |
| | TCTTTGAGTG GACAGCGGAG GACTGGACTG CCAGGCCAAG CATCAGGGGC TTCATCCTCA | 300 |
| | GGGCCGGTTA GAGCCCCTGA GGATTTAGGA GGAAGGGAAA CCAATGAAAA GCGTGCTGGT | 360 |
| | GGTGGCTCTC CTTGTCATTT TCCAGGTATG CCTGTGTCAA GATGAGGTCA CGGACGATTA | 420 |
| | CATCGGAGAC AACACCACAG TGGACTACAC TTTGTTCGAG TCTTTGTGCT CCAAGAAGGA | 480 |
| | CGTGCGGAAC TTTAAAGCCT GGTTCCTCCC TATCATGTAC TCCATCATTT GTTTCGTGGG | 540 |
| | CCTACTGGGC AATGGGCTGG TCGTGTTGAC CTATATCTAT TTCAAGAGGC TCAAGACCAT | 600 |
| | GACCGATACC TACCTGCTCA ACCTGGCGGT GGCAGACATC CTCTTCCTCC TGACCCTTCC | 660 |
| | CTTCTGGGCC TACAGCGCGG CCAAGTCCTG GGTCTTCGGT GTCCACTTTT GCAAGCTCAT | 720 |
| | CTTTGCCATC TACAAGATGA GCTTCTTCAG TGGCATGCTC CTACTTCTTT GCATCAGCAT | 780 |
| | TGACCGCTAC GTGGCCATCG TCCAGGCTGT CTCAGCTCAC CGCCACCGTG CCCGCGTCCT | 840 |
| | TCTCATCAGC AAGCSGTCCT GTGTGGGCAT CTGGATACTA GCCACAGTGC TCTCCATCCC | 900 |
| | AGAGCTCCTG TACAGTGACC TCCAGAGGAG CAGCAGTGAG CAAGCGATGC GATGCTCTCT | 960 |
| | CATCACAGAG CATGTGGAGG CCTTTATCAC CATCCAGGTG GCCCAGATGG TGATCGGCTT | 1020 |
| | TCTGGTCCCC CTGCTGGCCA TGAGCTTCTG TTACCTTGTC ATCATCCGCA CCCTGCTCCA | 1080 |
| | GGCACGCAAC TTTGAGCGCA ACAAGGCCAT CAAGGTGATC ATCGCTGTGG TCGTGGTCTT | 1140 |
| | CATAGTCTTC CAGCTGCCCT ACAATGGGGT GGTCCTGGCC CAGACGGTGG CCAACTTCAA | 1200 |
| | CATCACCAGT AGCACCTGTG AGCTCAGTAA GCAACTCAAC ATCGCCTACG ACGTCACCTA | 1260 |
| | CAGCCTGGCC TGCGTCCGCT GCTGCGTCAA CCCTTTCTTG TACGCCTTCA TCGGCGTCAA | 1320 |
| | GTTCCGCAAC GATCTCTTCA AGCTCTTCAA GGACCTGGGC TGCCTCAGCC AGGAGCAGCT | 1380 |
| | CCGGCAGTGG TCTTCCTGTC GGCACATCCG GCGCTCCTCC ATGAGTGTGG AGGCCGAGAC | 1440 |
| | CACCACCACC TTCTCCCCAT AGGCGACTCT TCTGCCTGGA CTAGAGGGAC CTCTCCCAGG | 1500 |
| | GTCCCTGGGG TGGGGATAGG GAGCAGATGC AATGACTCAG GACATCCCCC CGCCAAAAGC | 1560 |
| | TGCTCAGGGA AAAGCAGCTC TCCCCTCAGA GTGCAAGCCC CTGCTCCAGA AGATAGCTTC | 1620 |
| | ACCCCAATCC CAGCTACCTC AACCAATGCC AAAAAAAGAC AGGGCTGATA AGCTAACACC | 1680 |
| | AGACAGACAA CACTGGGAAA CAGAGGCTAT TGTCCCCTAA ACCAAAAACT GAAAGTGAAA | 1740 |
| | GTCCAGAAAC TGTTCCCACC TGCTGGAGTG AAGGGGCCAA GGAGGGTGAG TGCAAGGGGC | 1800 |
| | GTGGGAGTGG CCTGAAGAGT CCTCTGAATG AACCTTCTGG CCTCCCACAG ACTCAAATGC | 1860 |
| | TCAGACCAGC TCTTCCGAAA ACCAGGCCTT ATCTCCAAGA CCAGAGATAG TGGGGAGACT | 1920 |
| | TCTTGGCTTG GTGAGGAAAA GCGGACATCA GCTGGTCAAA CAAACTCTCT GAACCCCTCC | 1980 |
| | CTCCATCGTT TTCTICACTG TCCTCCAAGC CAGCGGGAAT GGCAGCTGCC ACGCCGCCCT | 2040 |
| | AAAAGCACAC TCATCCCCTC ACTTGCCGCG TCGCCCTCCC AGGCTCTCAA CAGGGGAGAG | 2100 |
| | TGTGGTGTTT CCTGCAGGCC AGGCCAGCTG CCTCCGCGTG ATCAAAGCCA CACTCTGGGC | 2160 |
| | TCCAGAGTGG GGATGACATG CACTCAGCTC TTGGCTCCAC TGGGATGGGA GGAGAGGACA | 2220 |
| | AGGGAAATGT CAGGGGGGGG GAGGGTGACA GTGGCCGCCC AAGGCCCACG AGCTTGTTCT | 2280 |
| | TTGTTCTTTG TCACAGGGAC TGAAAACCTC TCCTCATGTT CTGCTTTCGA TTCGTTAAGA | 2340 |
| | GAGCAACATT TTACCCACAC ACAGATAAAG TTTTCCCTTG AGGAAACAAC AGCTTTAAAA | 2400 |
| | GAAAAAGAAA AAAAAAGTCT TTGGTAAATG GCAAA | 2435 |
| SEQ ID NO: 666 | CTCTAGATGA GTCAGTGGAG GGCGGGTGGA GCGTTGAACC GTGAAGAGTG TGGTTGGGCG | 60 |
| CCR7 variant 4 | TAAACGTGGA CTTAAACTCA GGAGCTAAGG GGGAAACCAA TGAAAAGCGT GCTGGTGGTG | 120 |
| domain | GCTCTCCTTG TCATTTTCCA GGTATGCCTG TGTCAAGATG AGGTCACGGA CGATTACATC | 180 |
| | GGAGACAACA CCACAGTGGA CTACACTTTG TTCGAGTCTT TGTGCTCCAA GAAGGACGTG | 240 |
| | CGGAACTTTA AAGCCTGGTT CCTCCCTATC ATGTACTCCA TCATTTGTTT CGTGGGCCTA | 300 |
| | CTGGGCAATG GGCTGGTCGT GTTGACCTAT ATCTATTTCA AGAGGCTCAA GACCATGACC | 360 |
| | GATACCTACC TGCTCAACCT GGCGGTGGCA GACATCCTCT TCCTCCTGAC CCTTCCCTTC | 420 |
| | TGGGCCTACA GCGCGGCCAA GTCCTGGGTC TTCGGTGTCC ACTTTTGCAA GCTCATCTTT | 480 |
| | GCCATCTACA AGATGAGCTT CTTCAGTGGC ATGCTCCTAC TTCTTTGCAT CAGCATTGAC | 540 |
| | CGCTACGTGG CCATCGTCCA GGCTGTCTCA GCTCACCGCC ACCGTGCCCG CGTCCTTCTC | 600 |
| | ATCAGCAAGC TGTCCTGTGT GGGCATCTGG ATACTAGCCA CAGTGCTCTC CATCCCAGAG | 660 |
| | CTCCTGTACA GTGACCTCCA GAGGAGCAGC AGTGAGCAAG CGATGCGATG CTCTCTCATC | 720 |
| | ACAGAGCATG TGGAGGCCTT TATCACCATC CAGGTGGCCC AGATGGTGAT CGGCTTTCTG | 780 |
| | GTCCCCCTGC TGGCCATGAG CTTCTGTTAC CTTGTCATCA TCCGCACCCT GCTCCAGGCA | 840 |
| | CGCAACTTTG AGCGCAACAA GGCCATCAAG GTGATCATCG CTGTGGTCGT GGTCTTCATA | 900 |
| | GTCTTCCAGC TGCCCTACAA TGGGGTGGTC CTGGCCCAGA CGGTGGCCAA CTTCAACATC | 960 |
| | ACCAGTAGCA CCTGTGAGCT CAGTAAGCAA CTCAACATCG CCTACGACGT CACCTACAGC | 1020 |
| | CTGGCCTGCG TCCGCTGCTG CGTCAACCCT TTCTTGTACG CCTTCATCGG CGTCAAGTTC | 1080 |
| | CGCAACGATC TCTTCAAGCT CTTCAAGGAC CTGGGCTGCC TCAGCCAGGA GCAGCTCCGG | 1140 |
| | CAGTGGTCTT CCTGTCGGCA CATCCGGCGC TCCTCCATGA GTGTGGAGGC CGAGACCACC | 1200 |
| | ACCACCTTCT CCCCATAGGC GACTCTTCTG CCTGGACTAG AGGGACCTCT CCCAGGGTCC | 1260 |
| | CTGGGGTGGG GATAGGGAGC AGATGCAATG ACTCAGGACA TCCCCCCGCC AAAAGCTGCT | 1320 |
| | CAGGGAAAAG CAGCTCTCCC CTCAGAGTGC AAGCCCCTGC TCCAGAAGAT AGCTTCACCC | 1380 |
| | CAATCCCAGC TACCTCAACC AATGCCAAAA AAAGACAGGG CTGATAAGCT AACACCAGAC | 1440 |
| | AGACAACACT GGGAAACAGA GGCTATTGTC CCCTAAACCA AAAACTGAAA GTGAAAGTCC | 1500 |
| | AGAAACTGTT CCCACCTGCT GGAGTGAAGG GGCCAAGGAG GGTGAGTGCA AGGGGCGTGG | 1560 |
| | GAGTGGCCTG AAGAGTCCTC TGAATGAACC TTCTGGCCTC CCACAGACTC AAATGCTCAG | 1620 |
| | ACCAGCTCTT CCGAAAACCA GGCCTTATCT CCAAGACCAG AGATAGTGGG GAGACTTCTT | 1680 |
| | GGCTTGGTGA GGAAAAGCGG ACATCAGCTG GTCAAACAAA CTCTCTGAAC CCCTCCCTCC | 1740 |
| | ATCGTTTTCT TCACTGTCCT CCAAGCCAGC GGGAATGGCA GCTGCCACGC CGCCCTAAAA | 1800 |
| | GCACACTCAT CCCCTCACTT GCCGCGTCGC CCTCCCAGGC TCTCAACAGG GGAGAGTGTG | 1860 |
| | GTGTTTCCTG CAGGCCAGGC CAGCTGCCTC CGCGTGATCA AAGCCACACT CTGGGCTCCA | 1920 |
| | GAGTGGGGAT GACATGCACT CAGCTCTTGG CTCCACTGGG ATGGGAGGAG AGGACAAGGG | 1980 |
| | AAATGTCAGG GGCGGGGAGG GTGACAGTGG CCGCCCAAGG CCCACGAGCT TGTTCTTTGT | 2040 |
| | TCTTTGTCAC AGGGACTGAA AACCTCTCCT CATGTTCTGC TTTCGATTCG TTAAGAGAGC | 2100 |
| | AACATTTTAC CCACACACAG ATAAAGTTTT CCCTTGAGGA AACAACAGCT TTAAAAGAAA | 2160 |
| | AAGAAAAAAA AAGTCTTTGG TAAATGGCAA AGAAAAAGAA AAAAAAAGTC TTTGGTAAAT | 2220 |
| | GGCAAA | 2226 |
| SEQ ID NO: 667 | AGGAGAAGGT GCCTTAAACA GGTTCCCACG CATTTCCTGG CGCTATTGAG CTTGGAGCTG | 60 |
| CCR7 variant 5 | CCAAGGGCCT GCCTTCACTT GTGGCATCGC AGTTACTGAC TCTCCAGTGG GCCAGGCCCT | 120 |
| domain | ACCTAGCTGG GACCTGAGGG TCAGGATACG GGAAGAGGGC TACTGCCGCC CTGACTTGTA | 180 |
| | GGGAAACCAA TGAAAAGCGT GCTGGTGGTG GCTCTCCTTG TCATTTTCCA GGTATGCCTG | 240 |
| | TGTCAAGATG AGGTCACGGA CGATTACATC GGAGACAACA CCACAGTGGA CTACACTTTG | 300 |
| | TTCGAGTCTT TGTGCTCCAA GAAGGACGTG CGGAACTTTA AAGCCTGGTT CCTCCCTATC | 360 |
| | ATGTACTCCA TCATTTGTTT CGTGGGCCTA CTGGGCAATG GGCTGGTCGT GTTGACCTAT | 420 |
| | ATCTATTTCA AGAGGCTCAA GACCATGACC GATACCTACC TGCTCAACCT GGCGGTGGCA | 480 |
| | GACATCCTCT TCCTCCTGAC CCTTCCCTTC TGGGCCTACA GCGCGGCCAA GTCCTGGGTC | 540 |
| | TTCGGTGTCC ACTTTTGCAA GCTCATCTTT GCCATCTACA AGATGAGCTT CTTCAGTGGC | 600 |
| | ATGCTCCTAC TTCTTTGCAT CAGCATTGAC CGCTACGTGG CCATCGTCCA GGCTGTCTCA | 660 |
| | GCTCACCGCC ACCGTGCCCG CGTCCTTCTC ATCAGCAAGC TGTCCTGTGT GGGCATCTGG | 720 |
| | ATACTAGCCA CAGTGCTCTC CATCCCAGAG CTCCTGTACA GTGACCTCCA GAGGAGCAGC | 780 |
| | AGTGAGCAAG CGATGCGATG CTCTCTCATC ACAGAGCATG TGGAGGCCTT TATCACCATC | 840 |
| | CAGGTGGCCC AGATGGTGAT CGGCTTTCTG GTCCCCCTGC TGGCCATGAG CTTCTGTTAC | 900 |
| | CTTGTCATCA TCCGCACCCT GCTCCAGGCA CGCAACTTTG AGCGCAACAA GGCCATCAAG | 960 |
| | GTGATCATCG CTGTGGTCGT GGTCTTCATA GTCTTCCAGC TGCCCTACAA TGGGGTGGTC | 1020 |
| | CTGGCCCAGA CGGTGGCCAA CTTCAACATC ACCAGTAGCA CCTGTGAGCT CAGTAAGCAA | 1080 |
| | CTCAACATCG CCTACGACGT CACCTACAGC CTGGCCTGCG TCCGCTGCTG CGTCAACCCT | 1140 |
| | TTCTTGTACG CCTTCATCGG CGTCAAGTTC CGCAACGATC TCTTCAAGCT CTTCAAGGAC | 1200 |
| | CTGGGCTGCC TCAGCCAGGA GCAGCTCCGG CAGTGGTCTT CCTGTCGGCA CATCCGGCGC | 1260 |
| | TCCTCCATGA GTGTGGAGGC CGAGACCACC ACCACCTTCT CCCCATAGGC GACTCTTCTG | 1320 |
| | CCTGGACTAG AGGGACCTCT CCCAGGGTCC CTGGGGTGGG GATAGGGAGC AGATGCAATG | 1380 |
| | ACTCAGGACA TCCCCCCGCC AAAAGCTGCT CAGGGAAAAG CAGCTCTCCC CTCAGAGTGC | 1440 |
| | AAGCCCCTGC TCCAGAAGAT AGCTTCACCC CAATCCCAGC TACCTCAACC AATGCCAAAA | 1500 |
| | AAAGACAGGG CTGATAAGCT AACACCAGAC AGACAACACT GGGAAACAGA GGCTATTGTC | 1560 |
| | CCCTAAACCA AAAACTGAAA GTGAAAGTCC AGAAACTGTT CCCACCTGCT GGAGTGAAGG | 1620 |
| | GGCCAAGGAG GGTGAGTGCA AGGGGCGTGG GAGTGGCCTG AAGAGTCCTC TGAATGAACC | 1680 |
| | TTCTGGCCTC CCACAGACTC AAATGCTCAG ACCAGCTCTT CCGAAAACCA GGCCTTATCT | 1740 |
| | CCAAGACCAG AGATAGTGGG GAGACTTCTT GGCTTGGTGA GGAAAAGCGG ACATCAGCTG | 1800 |
| | GTCAAACAAA CTCTCTGAAC CCCTCCCTCC ATCGTTTTCT TCACTGTCCT CCAAGCCAGC | 1860 |
| | GGGAATGGCA GCTGCCACGC CGCCCTAAAA GCACACTCAT CCCCTCACTT GCCGCGTCGC | 1920 |
| | CCTCCCAGGC TCTCAACAGG GGAGAGTGTG GTGTTTCCTG CAGGCCAGGC CAGCTGCCTC | 1980 |
| | CGCGTGATCA AAGCCACACT CTGGGCTCCA GAGTGGGGAT GACATGCACT CAGCTCTTGG | 2040 |
| | CTCCACTGGG ATGGGAGGAG AGGACAAGGG AAATGTCAGG GGCGGGGAGG GTGACAGTGG | 2100 |
| | CCGCCCAAGG CCCACGAGCT TGTTCTTTGT TCTTTGTCAC AGGGACTGAA AACCTCTCCT | 2160 |
| | CATGTTCTGC TTTCGATTCG TTAAGAGAGC AACATTTTAC CCACACACAG ATAAAGTTTT | 2220 |
| | CCCTTGAGGA AACAACAGCT TTAAAAGAAA AAGAAAAAAA AAGTCTTTGG TAAATGGCAA | 2280 |
| | A | 2281 |
| SEQ ID NO: 668 | GTAGTGGGAG GATACCTCCA GAGAGGCTGC TGCTCATTGA GCTGCACTCA CATGAGGATA | 60 |
| CCR8 domain | CAGACTTTGT GAAGAAGGAA TTGGCAACAC TGAAACCTCC AGAACAAAGG CTGTCACTAA | 120 |
| | GGTCCCGCTG CCTTGATGGA TTATACACTT GACCTCAGTG TGACAACAGT GACCGACTAC | 180 |
| | TACTACCCTG ATATCTTCTC AAGCCCCTGT GATGCGGAAC TTATTCAGAC AAATGGCAAG | 240 |
| | TTGCTCCTTG CTGTCTTTTA TTGCCTCCTG TTTGTATTCA GTCTTCTGGG AAACAGCCTG | 300 |
| | GTCATCCTGG TCCTTGTGGT CTGCAAGAAG CTGAGGAGCA TCACAGATGT ATACCTCTTG | 360 |
| | AACCTGGCCC TGTCTGACCT GCTTTTTGTC TTCTCCTTCC CCTTTCAGAC CTACTATCTG | 420 |
| | CTGGACCAGT GGGTGTTTGG GACTGTAATG TGCAAAGTGG TGTCTGGCTT TTATTACATT | 480 |
| | GGCTTCTACA GCAGCATGTT TTTCATCACC CTCATGAGTG TGGACAGGTA CCTGGCTGTT | 540 |
| | GTCCATGCCG TGTATGCCCT AAAGGTGAGG ACGATCAGGA TGGGCACAAC GCTGTGCCTG | 600 |
| | GCAGTATGGC TAACCGCCAT TATGGCTACC ATCCCATTGC TAGTGTTTTA CCAAGTGGCC | 660 |
| | TCTGAAGATG GTGTTCTACA GTGTTATTCA TTTTACAATC AACAGACTTT GAAGTGGAAG | 720 |
| | ATCTTCACCA ACTTCAAAAT GAACATTTTA GGCTTGTTGA TCCCATTCAC CATCTTTATG | 780 |
| | TTCTGCTACA TTAAAATCCT GCACCAGCTG AAGAGGTGTC AAAACCACAA CAAGACCAAG | 840 |
| | GCCATCAGGT TGGTGCTCAT TGTGGTCATT GCATCTTTAC TTTTCTGGGT CCCATTCAAC | 900 |
| | GTGGTTCTTT TCCTCACTTC CTTGCACAGT ATGCACATCT TGGATGGATG TAGCATAAGC | 960 |
| | CAACAGCTGA CTTATGCCAC CCATGTCACA GAAATCATTT CCTTTACTCA CTGCTGTGTG | 1020 |
| | AACCCTGTTA TCTATGCTTT TGTTGGGGAG AAGTTCAAGA AACACCTCTC AGAAATATTT | 1080 |
| | CAGAAAAGTT GCAGCCAAAT CTTCAACTAC CTAGGAAGAC AAATGCCTAG GGAGAGCTGT | 1140 |
| | GAAAAGTCAT CATCCTGCCA GCAGCACTCC TCCCGTTCCT CCAGCGTAGA CTACATTTTG | 1200 |
| | TGAGGATCAA TGAAGACTAA ATATAAAAAA CATTTTCTTG AATGGCATGC TAGTAGCAGT | 1260 |
| | GAGCAAAGGT GTGGGTGTGA AAGGTTTCCA AAAAAAGTTC AGCATGAAGG ATGCCATATA | 1320 |
| | TGTTGTTGCC AACACTTGGA ACACAATGAC TAAAGACATA GTTGTGCATG CCTGGCACAA | 1380 |
| | CATCAAGCCT GTGASTGTGT TTATTGATGA TGTTGAACAA GTGGTAACTT TAAAGGATTC | 1440 |
| | TGTATGCCAA GTGAAAAAAA AAGATGTCTG ACCTCCTTAC ATAT | 1484 |

In an embodiment, a chemokine receptor of the present invention includes an amino acid domain encoded by the nucleotide sequence of SEQ ID NO:646, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:646. In an embodiment, a chemokine receptor of the present invention includes an amino acid domain encoded by a nucleotide sequence at least 98% identical to SEQ ID NO:646. In an embodiment, a chemokine receptor of the present invention includes an amino acid domain encoded by a nucleotide sequence at least 97% identical to SEQ ID NO:646. In an embodiment, a chemokine receptor of the present invention includes an amino acid domain encoded by a nucleotide sequence at least 96% identical to SEQ ID NO:646. In an embodiment, a chemokine receptor of the present invention includes an amino acid domain encoded by a nucleotide sequence at least 95% identical to SEQ ID NO:646. In an embodiment, a chemokine receptor of the present invention includes an amino acid domain encoded by a nucleotide sequence at least 90% identical to SEQ ID NO:646. In an embodiment, a chemokine receptor of the present invention includes an amino acid domain encoded by a nucleotide sequence at least 85% identical to SEQ ID NO:646. In an embodiment, a chemokine receptor of the present invention includes an amino acid domain encoded by a nucleotide sequence at least 80% identical to SEQ ID NO:646. In an embodiment, including the foregoing embodiments, SEQ ID NO:646 is codon-optimized to improve protein expression.

In an embodiment, a chemokine receptor of the present invention includes an amino acid domain encoded by the nucleotide sequence of SEQ ID NO:647, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:647. In an embodiment, a chemokine receptor of the present invention includes an amino acid domain encoded by a nucleotide sequence at least 98% identical to SEQ ID NO:647. In an embodiment, a chemokine receptor of the present invention includes an amino acid domain encoded by a nucleotide sequence at least 97% identical to SEQ ID NO:647. In an embodiment, a chemokine receptor of the present invention includes an amino acid domain encoded by a nucleotide sequence at least 96% identical to SEQ ID NO:647. In an embodiment, a chemokine receptor of the present invention includes an amino acid domain encoded by a nucleotide sequence at least 95% identical to SEQ ID NO:647. In an embodiment, a chemokine receptor of the present invention includes an amino acid domain encoded by a nucleotide sequence at least 90% identical to SEQ ID NO:647. In an embodiment, a chemokine receptor of the present invention includes an amino acid domain encoded by a nucleotide sequence at least 85% identical to SEQ ID NO:647. In an embodiment, a chemokine receptor of the present invention includes an amino acid domain encoded by a nucleotide sequence at least 80% identical to SEQ ID NO:647. In an embodiment, including the foregoing embodiments, SEQ ID NO:647 is codon-optimized to improve protein expression.

In an embodiment, a chemokine receptor of the present invention includes an amino acid domain encoded by the nucleotide sequence of SEQ ID NO:648, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:648. In an embodiment, a chemokine receptor of the present invention includes an amino acid domain encoded by a nucleotide sequence at least 98% identical to SEQ ID NO:648. In an embodiment, a chemokine receptor of the present invention includes an amino acid domain encoded by a nucleotide sequence at least 97% identical to SEQ ID NO:648. In an embodiment, a chemokine receptor of the present invention includes an amino acid domain encoded by a nucleotide sequence at least 96% identical to SEQ ID NO:648. In an embodiment, a chemokine receptor of the present invention includes an amino acid domain encoded by a nucleotide sequence at least 95% identical to SEQ ID NO:648. In an embodiment, a chemokine receptor of the present invention includes an amino acid domain encoded by a nucleotide sequence at least 90% identical to SEQ ID NO:648. In an embodiment, a chemokine receptor of the present invention includes an amino acid domain encoded by a nucleotide sequence at least 85% identical to SEQ ID NO:648. In an embodiment, a chemokine receptor of the present invention includes an amino acid domain encoded by a nucleotide sequence at least 80% identical to SEQ ID NO:648. In an embodiment, including the foregoing embodiments, SEQ ID NO:648 is codon-optimized to improve protein expression.

In an embodiment, a chemokine receptor of the present invention includes an amino acid domain encoded by the nucleotide sequence of SEQ ID NO:649, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:649. In an embodiment, a chemokine receptor of the present invention includes an amino acid domain encoded by a nucleotide sequence at least 98% identical to SEQ ID NO:649. In an embodiment, a chemokine receptor of the present invention includes an amino acid domain encoded by a nucleotide sequence at least 97% identical to SEQ ID NO:649. In an embodiment, a chemokine receptor of the present invention includes an amino acid domain encoded by a nucleotide sequence at least 96% identical to SEQ ID NO:649. In an embodiment, a chemokine receptor of the present invention includes an amino acid domain encoded by a nucleotide sequence at least 95% identical to SEQ ID NO:649. In an embodiment, a chemokine receptor of the present invention includes an amino acid domain encoded by a nucleotide sequence at least 90% identical to SEQ ID NO:649. In an embodiment, a chemokine receptor of the present invention includes an amino acid domain encoded by a nucleotide sequence at least 85% identical to SEQ ID NO:649. In an embodiment, a chemokine receptor of the present invention includes an amino acid domain encoded by a nucleotide sequence at least 80% identical to SEQ ID NO:649. In an embodiment, including the foregoing embodiments, SEQ ID NO:649 is codon-optimized to improve protein expression.

In an embodiment, a chemokine receptor of the present invention includes an amino acid domain encoded by the nucleotide sequence of SEQ ID NO:650, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:650. In an embodiment, a chemokine receptor of the present invention includes an amino acid domain encoded by a nucleotide sequence at least 98% identical to SEQ ID NO:650. In an embodiment, a chemokine receptor of the present invention includes an amino acid domain encoded by a nucleotide sequence at least 97% identical to SEQ ID NO:650. In an embodiment, a chemokine receptor of the present invention includes an amino acid domain encoded by a nucleotide sequence at least 96% identical to SEQ ID NO:650. In an embodiment, a chemokine receptor of the present invention includes an amino acid domain encoded by a nucleotide sequence at least 95% identical to SEQ ID NO:650. In an embodiment, a chemokine receptor of the present invention includes an amino acid domain encoded by a nucleotide sequence at least 90% identical to SEQ ID NO:650. In an embodiment, a chemokine receptor of the present invention includes an amino acid domain encoded by a nucleotide sequence at least 85% identical to SEQ ID NO:650. In an embodiment, a chemokine receptor of the present invention includes an amino acid domain encoded by a nucleotide sequence at least 80% identical to SEQ ID NO:650. In an embodiment, including the foregoing embodiments, SEQ ID NO:650 is codon-optimized to improve protein expression.

In an embodiment, a chemokine receptor of the present invention includes an amino acid domain encoded by the nucleotide sequence of SEQ ID NO:651, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:651. In an embodiment, a chemokine receptor of the present invention includes an amino acid domain encoded by a nucleotide sequence at least 98% identical to SEQ ID NO:651. In an embodiment, a chemokine receptor of the present invention includes an amino acid domain encoded by a nucleotide sequence at least 97% identical to SEQ ID NO:651. In an embodiment, a chemokine receptor of the present invention includes an amino acid domain encoded by a nucleotide sequence at least 96% identical to SEQ ID NO:651. In an embodiment, a chemokine receptor of the present invention includes an amino acid domain encoded by a nucleotide sequence at least 95% identical to SEQ ID NO:651. In an embodiment, a chemokine receptor of the present invention includes an amino acid domain encoded by a nucleotide sequence at least 90% identical to SEQ ID NO:651. In an embodiment, a chemokine receptor of the present invention includes an amino acid domain encoded by a nucleotide sequence at least 85% identical to SEQ ID NO:651. In an embodiment, a chemokine receptor of the present invention includes an amino acid domain encoded by a nucleotide sequence at least 80% identical to SEQ ID NO:651. In an embodiment, including the foregoing embodiments, SEQ ID NO:651 is codon-optimized to improve protein expression.

In an embodiment, a chemokine receptor of the present invention includes an amino acid domain encoded by the nucleotide sequence of SEQ ID NO:652, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:652. In an embodiment, a chemokine receptor of the present invention includes an amino acid domain encoded by a nucleotide sequence at least 98% identical to SEQ ID NO:652. In an embodiment, a chemokine receptor of the present invention includes an amino acid domain encoded by a nucleotide sequence at least 97% identical to SEQ ID NO:652. In an embodiment, a chemokine receptor of the present invention includes an amino acid domain encoded by a nucleotide sequence at least 96% identical to SEQ ID NO:652. In an embodiment, a chemokine receptor of the present invention includes an amino acid domain encoded by a nucleotide sequence at least 95% identical to SEQ ID NO:652. In an embodiment, a chemokine receptor of the present invention includes an amino acid domain encoded by a nucleotide sequence at least 90% identical to SEQ ID NO:652. In an embodiment, a chemokine receptor of the present invention includes an amino acid domain encoded by a nucleotide sequence at least 85% identical to SEQ ID NO:652. In an embodiment, a chemokine receptor of the present invention includes an amino acid domain encoded by a nucleotide sequence at least 80% identical to SEQ ID NO:652. In an embodiment, including the foregoing embodiments, SEQ ID NO:652 is codon-optimized to improve protein expression.

In an embodiment, a chemokine receptor of the present invention includes an amino acid domain encoded by the nucleotide sequence of SEQ ID NO:653, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:653. In an embodiment, a chemokine receptor of the present invention includes an amino acid domain encoded by a nucleotide sequence at least 98% identical to SEQ ID NO:653. In an embodiment, a chemokine receptor of the present invention includes an amino acid domain encoded by a nucleotide sequence at least 97% identical to SEQ ID NO:653. In an embodiment, a chemokine receptor of the present invention includes an amino acid domain encoded by a nucleotide sequence at least 96% identical to SEQ ID NO:653. In an embodiment, a chemokine receptor of the present invention includes an amino acid domain encoded by a nucleotide sequence at least 95% identical to SEQ ID NO:653. In an embodiment, a chemokine receptor of the present invention includes an amino acid domain encoded by a nucleotide sequence at least 90% identical to SEQ ID NO:653. In an embodiment, a chemokine receptor of the present invention includes an amino acid domain encoded by a nucleotide sequence at least 85% identical to SEQ ID NO:653. In an embodiment, a chemokine receptor of the present invention includes an amino acid domain encoded by a nucleotide sequence at least 80% identical to SEQ ID NO:653. In an embodiment, including the foregoing embodiments, SEQ ID NO:653 is codon-optimized to improve protein expression.

In an embodiment, a chemokine receptor of the present invention includes an amino acid domain encoded by the nucleotide sequence of SEQ ID NO:654, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:654. In an embodiment, a chemokine receptor of the present invention includes an amino acid domain encoded by a nucleotide sequence at least 98% identical to SEQ ID NO:654. In an embodiment, a chemokine receptor of the present invention includes an amino acid domain encoded by a nucleotide sequence at least 97% identical to SEQ ID NO:654. In an embodiment, a chemokine receptor of the present invention includes an amino acid domain encoded by a nucleotide sequence at least 96% identical to SEQ ID NO:654. In an embodiment, a chemokine receptor of the present invention includes an amino acid domain encoded by a nucleotide sequence at least 95% identical to SEQ ID NO:654. In an embodiment, a chemokine receptor of the present invention includes an amino acid domain encoded by a nucleotide sequence at least 90% identical to SEQ ID NO:654. In an embodiment, a chemokine receptor of the present invention includes an amino acid domain encoded by a nucleotide sequence at least 85% identical to SEQ ID NO:654. In an embodiment, a chemokine receptor of the present invention includes an amino acid domain encoded by a nucleotide sequence at least 80% identical to SEQ ID NO:654. In an embodiment, including the foregoing embodiments, SEQ ID NO:654 is codon-optimized to improve protein expression.

In an embodiment, a chemokine receptor of the present invention includes an amino acid domain encoded by the nucleotide sequence of SEQ ID NO:655, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:655. In an embodiment, a chemokine receptor of the present invention includes an amino acid domain encoded by a nucleotide sequence at least 98% identical to SEQ ID NO:655. In an embodiment, a chemokine receptor of the present invention includes an amino acid domain encoded by a nucleotide sequence at least 97% identical to SEQ ID NO:655 In an embodiment, a chemokine receptor of the present invention includes an amino acid domain encoded by a nucleotide sequence at least 96% identical to SEQ ID NO:655. In an embodiment, a chemokine receptor of the present invention includes an amino acid domain encoded by a nucleotide sequence at least 95% identical to SEQ ID NO:655. In an embodiment, a chemokine receptor of the present invention includes an amino acid domain encoded by a nucleotide sequence at least 90% identical to SEQ ID NO:655. In an embodiment, a chemokine receptor of the present invention includes an amino acid domain encoded by a nucleotide sequence at least 85% identical to SEQ ID NO:655. In an embodiment, a chemokine receptor of the present invention includes an amino acid domain encoded by a nucleotide sequence at least 80% identical to SEQ ID NO:655. In an embodiment, including the foregoing embodiments, SEQ ID NO:655 is codon-optimized to improve protein expression.

In an embodiment, a chemokine receptor of the present invention includes an amino acid domain encoded by the nucleotide sequence of SEQ ID NO:656, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:656. In an embodiment, a chemokine receptor of the present invention includes an amino acid domain encoded by a nucleotide sequence at least 98% identical to SEQ ID NO:656. In an embodiment, a chemokine receptor of the present invention includes an amino acid domain encoded by a nucleotide sequence at least 97% identical to SEQ ID NO:656. In an embodiment, a chemokine receptor of the present invention includes an amino acid domain encoded by a nucleotide sequence at least 96% identical to SEQ ID NO:656. In an embodiment, a chemokine receptor of the present invention includes an amino acid domain encoded by a nucleotide sequence at least 95% identical to SEQ ID NO:656. In an embodiment, a chemokine receptor of the present invention includes an amino acid domain encoded by a nucleotide sequence at least 90% identical to SEQ ID NO:656. In an embodiment, a chemokine receptor of the present invention includes an amino acid domain encoded by a nucleotide sequence at least 85% identical to SEQ ID NO:656. In an embodiment, a chemokine receptor of the present invention includes an amino acid domain encoded by a nucleotide sequence at least 80% identical to SEQ ID NO:656. In an embodiment, including the foregoing embodiments, SEQ ID NO:656 is codon-optimized to improve protein expression.

In an embodiment, a chemokine receptor of the present invention includes an amino acid domain encoded by the nucleotide sequence of SEQ ID NO:657, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:657. In an embodiment, a chemokine receptor of the present invention includes an amino acid domain encoded by a nucleotide sequence at least 98% identical to SEQ ID NO:657. In an embodiment, a chemokine receptor of the present invention includes an amino acid domain encoded by a nucleotide sequence at least 97% identical to SEQ ID NO:657. In an embodiment, a chemokine receptor of the present invention includes an amino acid domain encoded by a nucleotide sequence at least 96% identical to SEQ ID NO:657. In an embodiment, a chemokine receptor of the present invention includes an amino acid domain encoded by a nucleotide sequence at least 95% identical to SEQ ID NO:657. In an embodiment, a chemokine receptor of the present invention includes an amino acid domain encoded by a nucleotide sequence at least 90% identical to SEQ ID NO:657. In an embodiment, a chemokine receptor of the present invention includes an amino acid domain encoded by a nucleotide sequence at least 85% identical to SEQ ID NO:657. In an embodiment, a chemokine receptor of the present invention includes an amino acid domain encoded by a nucleotide sequence at least 80% identical to SEQ ID NO:657. In an embodiment, including the foregoing embodiments, SEQ ID NO:657 is codon-optimized to improve protein expression.

In an embodiment, a chemokine receptor of the present invention includes an amino acid domain encoded by the nucleotide sequence of SEQ ID NO:658, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:658. In an embodiment, a chemokine receptor of the present invention includes an amino acid domain encoded by a nucleotide sequence at least 98% identical to SEQ ID NO:658. In an embodiment, a chemokine receptor of the present invention includes an amino acid domain encoded by a nucleotide sequence at least 97% identical to SEQ ID NO:658. In an embodiment, a chemokine receptor of the present invention includes an amino acid domain encoded by a nucleotide sequence at least 96% identical to SEQ ID NO:658. In an embodiment, a chemokine receptor of the present invention includes an amino acid domain encoded by a nucleotide sequence at least 95% identical to SEQ ID NO:658. In an embodiment, a chemokine receptor of the present invention includes an amino acid domain encoded by a nucleotide sequence at least 90% identical to SEQ ID NO:658. In an embodiment, a chemokine receptor of the present invention includes an amino acid domain encoded by a nucleotide sequence at least 85% identical to SEQ ID NO:658. In an embodiment, a chemokine receptor of the present invention includes an amino acid domain encoded by a nucleotide sequence at least 80% identical to SEQ ID NO:658. In an embodiment, including the foregoing embodiments, SEQ ID NO:658 is codon-optimized to improve protein expression.

In an embodiment, a chemokine receptor of the present invention includes an amino acid domain encoded by the nucleotide sequence of SEQ ID NO:659, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:659. In an embodiment, a chemokine receptor of the present invention includes an amino acid domain encoded by a nucleotide sequence at least 98% identical to SEQ ID NO:659. In an embodiment, a chemokine receptor of the present invention includes an amino acid domain encoded by a nucleotide sequence at least 97% identical to SEQ ID NO:659. In an embodiment, a chemokine receptor of the present invention includes an amino acid domain encoded by a nucleotide sequence at least 96% identical to SEQ ID NO:659. In an embodiment, a chemokine receptor of the present invention includes an amino acid domain encoded by a nucleotide sequence at least 95% identical to SEQ ID NO:659. In an embodiment, a chemokine receptor of the present invention includes an amino acid domain encoded by a nucleotide sequence at least 90% identical to SEQ ID NO:659. In an embodiment, a chemokine receptor of the present invention includes an amino acid domain encoded by a nucleotide sequence at least 85% identical to SEQ ID NO:659. In an embodiment, a chemokine receptor of the present invention includes an amino acid domain encoded by a nucleotide sequence at least 80% identical to SEQ ID NO:659. In an embodiment, including the foregoing embodiments, SEQ ID NO:659 is codon-optimized to improve protein expression.

In an embodiment, a chemokine receptor of the present invention includes an amino acid domain encoded by the nucleotide sequence of SEQ ID NO:660, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:660. In an embodiment, a chemokine receptor of the present invention includes an amino acid domain encoded by a nucleotide sequence at least 98% identical to SEQ ID NO:660. In an embodiment, a chemokine receptor of the present invention includes an amino acid domain encoded by a nucleotide sequence at least 97% identical to SEQ ID NO:660. In an embodiment, a chemokine receptor of the present invention includes an amino acid domain encoded by a nucleotide sequence at least 96% identical to SEQ ID NO:660. In an embodiment, a chemokine receptor of the present invention includes an amino acid domain encoded by a nucleotide sequence at least 95% identical to SEQ ID NO:660. In an embodiment, a chemokine receptor of the present invention includes an amino acid domain encoded by a nucleotide sequence at least 90% identical to SEQ ID NO:660. In an embodiment, a chemokine receptor of the present invention includes an amino acid domain encoded by a nucleotide sequence at least 85% identical to SEQ ID NO:660. In an embodiment, a chemokine receptor of the present invention includes an amino acid domain encoded by a nucleotide sequence at least 80% identical to SEQ ID NO:660. In an embodiment, including the foregoing embodiments, SEQ ID NO:660 is codon-optimized to improve protein expression.

In an embodiment, a chemokine receptor of the present invention includes an amino acid domain encoded by the nucleotide sequence of SEQ ID NO:661, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:661. In an embodiment, a chemokine receptor of the present invention includes an amino acid domain encoded by a nucleotide sequence at least 98% identical to SEQ ID NO:661. In an embodiment, a chemokine receptor of the present invention includes an amino acid domain encoded by a nucleotide sequence at least 97% identical to SEQ ID NO:661. In an embodiment, a chemokine receptor of the present invention includes an amino acid domain encoded by a nucleotide sequence at least 96% identical to SEQ ID NO:661. In an embodiment, a chemokine receptor of the present invention includes an amino acid domain encoded by a nucleotide sequence at least 95% identical to SEQ ID NO:661. In an embodiment, a chemokine receptor of the present invention includes an amino acid domain encoded by a nucleotide sequence at least 90% identical to SEQ ID NO:661. In an embodiment, a chemokine receptor of the present invention includes an amino acid domain encoded by a nucleotide sequence at least 85% identical to SEQ ID NO:661. In an embodiment, a chemokine receptor of the present invention includes an amino acid domain encoded by a nucleotide sequence at least 80% identical to SEQ ID NO:661. In an embodiment, including the foregoing embodiments, SEQ ID NO:661 is codon-optimized to improve protein expression.

In an embodiment, a chemokine receptor of the present invention includes an amino acid domain encoded by the nucleotide sequence of SEQ ID NO:662, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:662. In an embodiment, a chemokine receptor of the present invention includes an amino acid domain encoded by a nucleotide sequence at least 98% identical to SEQ ID NO:662. In an embodiment, a chemokine receptor of the present invention includes an amino acid domain encoded by a nucleotide sequence at least 97% identical to SEQ ID NO:662. In an embodiment, a chemokine receptor of the present invention includes an amino acid domain encoded by a nucleotide sequence at least 96% identical to SEQ ID NO:662. In an embodiment, a chemokine receptor of the present invention includes an amino acid domain encoded by a nucleotide sequence at least 95% identical to SEQ ID NO:662. In an embodiment, a chemokine receptor of the present invention includes an amino acid domain encoded by a nucleotide sequence at least 90% identical to SEQ ID NO:662. In an embodiment, a chemokine receptor of the present invention includes an amino acid domain encoded by a nucleotide sequence at least 85% identical to SEQ ID NO:662. In an embodiment, a chemokine receptor of the present invention includes an amino acid domain encoded by a nucleotide sequence at least 80% identical to SEQ ID NO:662. In an embodiment, including the foregoing embodiments, SEQ ID NO:662 is codon-optimized to improve protein expression.

In an embodiment, a chemokine receptor of the present invention includes an amino acid domain encoded by the nucleotide sequence of SEQ ID NO:663, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:663. In an embodiment, a chemokine receptor of the present invention includes an amino acid domain encoded by a nucleotide sequence at least 98% identical to SEQ ID NO:663. In an embodiment, a chemokine receptor of the present invention includes an amino acid domain encoded by a nucleotide sequence at least 97% identical to SEQ ID NO:663. In an embodiment, a chemokine receptor of the present invention includes an amino acid domain encoded by a nucleotide sequence at least 96% identical to SEQ ID NO:663. In an embodiment, a chemokine receptor of the present invention includes an amino acid domain encoded by a nucleotide sequence at least 95% identical to SEQ ID NO:663. In an embodiment, a chemokine receptor of the present invention includes an amino acid domain encoded by a nucleotide sequence at least 90% identical to SEQ ID NO:663. In an embodiment, a chemokine receptor of the present invention includes an amino acid domain encoded by a nucleotide sequence at least 85% identical to SEQ ID NO:663. In an embodiment, a chemokine receptor of the present invention includes an amino acid domain encoded by a nucleotide sequence at least 80% identical to SEQ ID NO:663. In an embodiment, including the foregoing embodiments, SEQ ID NO:663 is codon-optimized to improve protein expression.

In an embodiment, a chemokine receptor of the present invention includes an amino acid domain encoded by the nucleotide sequence of SEQ ID NO:664, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:664. In an embodiment, a chemokine receptor of the present invention includes an amino acid domain encoded by a nucleotide sequence at least 98% identical to SEQ ID NO:664. In an embodiment, a chemokine receptor of the present invention includes an amino acid domain encoded by a nucleotide sequence at least 97% identical to SEQ ID NO:664. In an embodiment, a chemokine receptor of the present invention includes an amino acid domain encoded by a nucleotide sequence at least 96% identical to SEQ ID NO:664. In an embodiment, a chemokine receptor of the present invention includes an amino acid domain encoded by a nucleotide sequence at least 95% identical to SEQ ID NO:664. In an embodiment, a chemokine receptor of the present invention includes an amino acid domain encoded by a nucleotide sequence at least 90% identical to SEQ ID NO:664. In an embodiment, a chemokine receptor of the present invention includes an amino acid domain encoded by a nucleotide sequence at least 85% identical to SEQ ID NO:664. In an embodiment, a chemokine receptor of the present invention includes an amino acid domain encoded by a nucleotide sequence at least 80% identical to SEQ ID NO:664. In an embodiment, including the foregoing embodiments, SEQ ID NO:664 is codon-optimized to improve protein expression.

In an embodiment, a chemokine receptor of the present invention includes an amino acid domain encoded by the nucleotide sequence of SEQ ID NO:665, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:665. In an embodiment, a chemokine receptor of the present invention includes an amino acid domain encoded by a nucleotide sequence at least 98% identical to SEQ ID NO:665. In an embodiment, a chemokine receptor of the present invention includes an amino acid domain encoded by a nucleotide sequence at least 97% identical to SEQ ID NO:665. In an embodiment, a chemokine receptor of the present invention includes an amino acid domain encoded by a nucleotide sequence at least 96% identical to SEQ ID NO:665. In an embodiment, a chemokine receptor of the present invention includes an amino acid domain encoded by a nucleotide sequence at least 95% identical to SEQ ID NO:665. In an embodiment, a chemokine receptor of the present invention includes an amino acid domain encoded by a nucleotide sequence at least 90% identical to SEQ ID NO:665. In an embodiment, a chemokine receptor of the present invention includes an amino acid domain encoded by a nucleotide sequence at least 85% identical to SEQ ID NO:665. In an embodiment, a chemokine receptor of the present invention includes an amino acid domain encoded by a nucleotide sequence at least 80% identical to SEQ ID NO:665. In an embodiment, including the foregoing embodiments, SEQ ID NO:665 is codon-optimized to improve protein expression.

In an embodiment, a chemokine receptor of the present invention includes an amino acid domain encoded by the nucleotide sequence of SEQ ID NO:666, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:666. In an embodiment, a chemokine receptor of the present invention includes an amino acid domain encoded by a nucleotide sequence at least 98% identical to SEQ ID NO:666. In an embodiment, a chemokine receptor of the present invention includes an amino acid domain encoded by a nucleotide sequence at least 97% identical to SEQ ID NO:666. In an embodiment, a chemokine receptor of the present invention includes an amino acid domain encoded by a nucleotide sequence at least 96% identical to SEQ ID NO:666. In an embodiment, a chemokine receptor of the present invention includes an amino acid domain encoded by a nucleotide sequence at least 95% identical to SEQ ID NO:666. In an embodiment, a chemokine receptor of the present invention includes an amino acid domain encoded by a nucleotide sequence at least 90% identical to SEQ ID NO:666. In an embodiment, a chemokine receptor of the present invention includes an amino acid domain encoded by a nucleotide sequence at least 85% identical to SEQ ID NO:666. In an embodiment, a chemokine receptor of the present invention includes an amino acid domain encoded by a nucleotide sequence at least 80% identical to SEQ ID NO:666. In an embodiment, including the foregoing embodiments, SEQ ID NO:666 is codon-optimized to improve protein expression.

In an embodiment, a chemokine receptor of the present invention includes an amino acid domain encoded by the nucleotide sequence of SEQ ID NO:667, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:667. In an embodiment, a chemokine receptor of the present invention includes an amino acid domain encoded by a nucleotide sequence at least 98% identical to SEQ ID NO:667. In an embodiment, a chemokine receptor of the present invention includes an amino acid domain encoded by a nucleotide sequence at least 97% identical to SEQ ID NO:667. In an embodiment, a chemokine receptor of the present invention includes an amino acid domain encoded by a nucleotide sequence at least 96% identical to SEQ ID NO:667. In an embodiment, a chemokine receptor of the present invention includes an amino acid domain encoded by a nucleotide sequence at least 95% identical to SEQ ID NO:667. In an embodiment, a chemokine receptor of the present invention includes an amino acid domain encoded by a nucleotide sequence at least 90% identical to SEQ ID NO:667. In an embodiment, a chemokine receptor of the present invention includes an amino acid domain encoded by a nucleotide sequence at least 85% identical to SEQ ID NO:667. In an embodiment, a chemokine receptor of the present invention includes an amino acid domain encoded by a nucleotide sequence at least 80% identical to SEQ ID NO:667. In an embodiment, including the foregoing embodiments, SEQ ID NO:667 is codon-optimized to improve protein expression.

In an embodiment, a chemokine receptor of the present invention includes an amino acid domain encoded by the nucleotide sequence of SEQ ID NO:668, or conservative amino acid substitutions thereof, or fragments, variants, or derivatives thereof. In an embodiment, a chemokine receptor of the present invention includes an amino acid domain encoded by a nucleotide sequence at least 99% identical to SEQ ID NO:668. In an embodiment, a chemokine receptor of the present invention includes an amino acid domain encoded by a nucleotide sequence at least 98% identical to SEQ ID NO:668. In an embodiment, a chemokine receptor of the present invention includes an amino acid domain encoded by a nucleotide sequence at least 97% identical to SEQ ID NO:668. In an embodiment, a chemokine receptor of the present invention includes an amino acid domain encoded by a nucleotide sequence at least 96% identical to SEQ ID NO:668. In an embodiment, a chemokine receptor of the present invention includes an amino acid domain encoded by a nucleotide sequence at least 95% identical to SEQ ID NO:668. In an embodiment, a chemokine receptor of the present invention includes an amino acid domain encoded by a nucleotide sequence at least 90% identical to SEQ ID NO:668. In an embodiment, a chemokine receptor of the present invention includes an amino acid domain encoded by a nucleotide sequence at least 85% identical to SEQ ID NO:668. In an embodiment, a chemokine receptor of the present invention includes an amino acid domain encoded by a nucleotide sequence at least 80% identical to SEQ ID NO:668. In an embodiment, including the foregoing embodiments, SEQ ID NO:668 is codon-optimized to improve protein expression.

### B. Gene Expression Methods for Chemokine Receptors

The gene expression methods described elsewhere herein or in the art, including but not limited to lentiviral, retroviral, and transposon-based systems, may be used to provide for stable expression of chemokine receptors in TILs, MILs, or PBLs, such as the exemplary systems described in Section VIII.D above, and including the promoters, self-cleaving peptides, linkers, regulatory elements or domains, and other vector components or domains.

Nucleotide sequences of vectors encoding exemplary CCRs of the present invention are provided in Table 66. In an embodiment, a nucleotide sequence in Table 66 is codon-optimized to improve protein expression. In an embodiment, a nucleotide sequence in Table 66 is further modified to include alternative promoter or regulatory domains, as described elsewhere herein. In an embodiment, a nucleotide sequence in Table 66 is used in a retroviral expression system. In an embodiment, a nucleotide sequence in Table 66 is used in a retroviral expression system using additional plasmids. Additional details are described in Hawley, et al., Gene Ther. 1994, 1, 136-38; the disclosures of which is incorporated by reference herein.

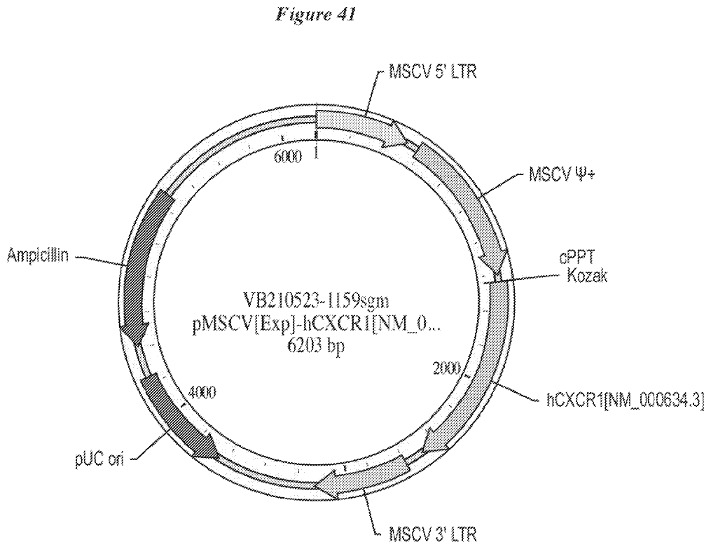

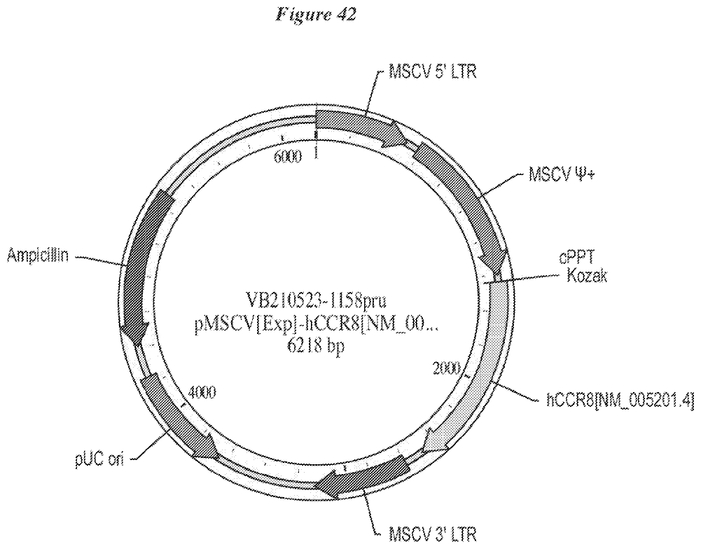

Exemplary vector designs for the vectors provided in Table 66 are provided in FIGS. 41 and 42. In an embodiment, a chemokine receptor encoded by the vector shown in FIG. 41 is used to genetically modify a TIL product of the present invention as described herein. In an embodiment, a chemokine receptor encoded by the vector shown in FIG. 41 is used to genetically modify a TIL product of the present invention as described herein. In an embodiment, a chemokine receptor encoded by the vector shown in FIG. 42 is used to genetically modify a TIL product of the present invention as described herein.

TABLE 66
Nucleotide sequences of exemplary vectors for expression of chemokine
receptors.

| Identifier | Sequence (One-Letter Nucleotide Symbols) | |
|---|---|---|
| SEQ ID NO: 669 | AATGAAAGAC CCCACCTGTA GGTTTGGCAA GCTAGCTTAA GTAACGCCAT TTTGCAAGGC | 60 |
| MSCV CXCR1 | ATGGAAAATA CATAACTGAG AATAGAGAAG TTCAGATCAA GGTTAGGAAC AGAGAGACAG | 120 |
| retroviral | CAGAATATGG GCCAAACAGG ATATCTGTGG TAAGCAGTTC CTGCCCCGGC TCAGGGCCAA | 180 |
| vector | GAACAGATGG TCCCCAGATG CGGTCCCGCC CTCAGCAGTT TCTAGAGAAC CATCAGATGT | 240 |
| | TTCCAGGGTG CCCCAAGGAC CTGAAATGAC CCTGTGCCTT ATTTGAACTA ACCAATCAGT | 300 |
| | TCGCTTCTCG CTTCTGTTCG CGCGCTTCTG CTCCCCGAGC TCAATAAAAG AGCCCACAAC | 360 |
| | CCCTCACTCG GCGCGCCAGT CCTCCGATAG ACTGCGTCGC CCGGGTACCC GTATTCCCAA | 420 |
| | TAAAGCCTCT TGCTGTTTGC ATCCGAATCG TGGACTCGCT GATCCTTGGG AGGGTCTCCT | 480 |
| | CAGATTGATT GACTGCCCAC CTCGGGGGTC TTTCATTTGG AGGTTCCACC GAGATTTGGA | 540 |
| | GACCCCTGCC CAGGGACCAC CGACCCCCCC GCCGGGAGGT AAGCTGGCCA GCGGTCGTTT | 600 |
| | CGTGTCTGTC TCTGTCTTTG TGCGTGTTTG TGCCGGCATC TAATGTTTGC GCCTGCGTCT | 660 |
| | GTACTAGTTA GCTAACTAGC TCTGTATCTG GCGGACCCGT GGTGGAACTG ACGAGTTCTG | 720 |
| | AACACCCGGC CGCAACCCTG GGAGACGTCC CAGGGACTTT GGGGGCCGTT TTTGTGGCCC | 780 |
| | GACCTGAGGA AGGGAGTCGA TGTGGAATCC GACCCCGTCA GGATATGTGG TTCTGGTAGG | 840 |
| | AGACGAGAAC CTAAAACAGT TCCCGCCTCC GTCTGAATTT TTGCTTTCGG TTTGGAACCG | 900 |
| | AAGCCGCGCG TCTTGTCTGC TGCAGCGCTG CAGCATCGTT CTGTGTTGTC TCTGTCTGAC | 960 |
| | TGTGTTTCTG TATTTGTCTG AAAATTAGGG CCAGACTGTT ACCACTCCCT TAAGTTTGAC | 1020 |
| | CTTAGGTCAC TGGAAAGATG TCGAGCGGAT CGCTCACAAC CAGTCGGTAG ATGTCAAGAA | 1080 |
| | GAGACGTTGG GTTACCTTCT GCTCTGCAGA ATGGCCAACC TTTAACGTCG GATGGCCGCG | 1140 |
| | AGACGGCACC TTTAACCGAG ACCTCATCAC CCAGGTTAAG ATCAAGGTCT TTTCACCTGG | 1200 |
| | CCCGCATGGA CACCCAGACC AGGTCCCCTA CATCGTGACC TGGGAAGCCT TGGCTTTTGA | 1260 |
| | CCCCCCTCCC TGGGTCAAGC CCTTTGTACA CCCTAAGCCT CCGCCTCCTC TTCCTCCATC | 1320 |
| | CGCCCCGTCT CTCCCCCTTG AACCTCCTCG TTCGACCCCG CCTCGATCCT CCCTTTATCC | 1380 |
| | AGCCCTCACT CCTTCTCTAG GCGCCGGAAT TCCAAGTTTG TACAAAAAAG CAGGCTGCCA | 1440 |
| | CCATGTCAAA TATTACAGAT CCACAGATGT GGGATTTTGA TGATCTAAAT TTCACTGGCA | 1500 |
| | TGCCACCTGC AGATGAAGAT TACAGCCCCT GTATGCTAGA AACTGAGACA CTCAACAAGT | 1560 |
| | ATGTTGTGAT CATCGCCTAT GCCCTAGTGT TCCTGCTGAG CCTGCTGGGA AACTCCCTGG | 1620 |
| | TGATGCTGGT CATCTTATAC AGCAGGGTCG GCCGCTCCGT CACTGATGTC TACCTGCTGA | 1680 |
| | ACCTGGCCTT GGCCGACCTA CTCTTTGCCC TGACCTTGCC CATCTGGGCC GCCTCCAAGG | 1740 |
| | TGAATGGCTG GATTTTTGGC ACATTCCTGT GCAAGGTGGT CTCACTCCTG AAGGAAGTCA | 1800 |
| | ACTTCTACAG TGGCATCCTG CTGTTGGCCT GCATCAGTGT GGACCGTTAC CTGGCCATTG | 1860 |
| | TCCATGCCAC ACGCACACTG ACCCAGAAGC GTCACTTGGT CAAGTTTGTT TGTCTTGGCT | 1920 |
| | GCTGGGGACT GTCTATGAAT CTGTCCCTGC CCTTCTTCCT TTTCCGCCAG GCTTACCATC | 1980 |
| | CAAACAATTC CAGTCCAGTT TGCTATGAGG TCCTGGGAAA TGACACAGCA AAATGGCGGA | 2040 |
| | TGGTGTTGCG GATCCTGCCT CACACCTTTG GCTTCATCGT GCCGCTGTTT GTCATGCTGT | 2100 |
| | TCTGCTATGG ATTCACCCTG CGTACACTGT TTAAGGCCCA CATGGGGCAG AAGCACCGAG | 2160 |
| | CCATGAGGGT CATCTTTGCT GTCGTCCTCA TCTTCCTGCT TTGCTGGCTG CCCTACAACC | 2220 |
| | TGGTCCTGCT GGCAGACACC CTCATGAGGA CCCAGGTGAT CCAGGAGAGC TGTGAGCGCC | 2280 |
| | GCAACAACAT CGGCCGGGCC CTGGATGCCA CTGAGATTCT GGGATTTCTC CATAGCTGCC | 2340 |
| | TCAACCCCAT CATCTACGCC TTCATCGGCC AAAATTTTCG CCATGGATTC CTCAAGATCC | 2400 |
| | TGGCTATGCA TGGCCTGGTC AGCAAGGAGT TCTTGGCACG TCATCGTGTT ACCTCCTACA | 2460 |
| | CTTCTTCGTC TGTCAATGTC TCTTCCAACC TCTGAACCCA GCTTTCTTGT ACAAAGTGGC | 2520 |
| | TCGAGAGATC CGTCGACCTG CAGCCAAGCT TATCGATAAA ATAAAAGATT TTATTTAGTC | 2580 |
| | TCCAGAAAAA GGGGGGAATG AAAGACCCCA CCTGTAGGTT TGGCAAGCTA GCTTAAGTAA | 2640 |
| | CGCCATTTTG CAAGGCATGG AAAATACATA ACTGAGAATA GAGAAGTTCA GATCAAGGTT | 2700 |
| | AGGAACAGAG AGACAGCAGA ATATGGGCCA AACAGGATAT CTGTGGTAAG CAGTTCCTGC | 2760 |
| | CCCGGCTCAG GGCCAAGAAC AGATGGTCCC CAGATGCGGT CCCGCCCTCA GCAGTTTCTA | 2820 |
| | GAGAACCATC AGATGTTTCC AGGGTGCCCC AAGGACCTGA AATGACCCTG TGCCTTATTT | 2880 |
| | GAACTAACCA ATCAGTTCGC TTCTCGCTTC TGTTCGCGCG CTTCTGCTCC CCGAGCTCAA | 2940 |
| | TAAAAGAGCC CACAACCCCT CACTCGGCGC GCCAGTCCTC CGATAGACTG CGTCGCCCGG | 3000 |
| | GTACCCGTGT ATCCAATAAA CCCTCTTGCA GTTGCATCCG ACTTGTGGTC TCGCTGTTCC | 3060 |
| | TTGGGAGGGT CTCCTCTGAG TGATTGACTA CCCGTCAGCG GGGGTCTTTC ATGGGTAACA | 3120 |
| | GTTTCTTGAA GTTGGAGAAC AACATTCTGA GGGTAGGAGT CGAATATTAA GTAATCCTGA | 3180 |
| | CTCAATTAGC CACTGTTTTG AATCCACATA CTCCAATACT CCTGAAATAG TTCATTATGG | 3240 |
| | ACAGCGCAGA AAGAGCTGGG GAGAATTGTG AAATTGTTAT CCGCTCACAA TTCCACACAA | 3300 |
| | CATACGAGCC GGAAGCATAA AGTGTAAAGC CTGGGGTGCC TAATGAGTGA GCTAACTCAC | 3360 |
| | ATTAATTGCG TTGCGCTCAC TGCCCGCTTT CCAGTCGGGA AACCTGTCGT GCCAGCTGCA | 3420 |
| | TTAATGAATC GGCCAACGCG CGGGGAGAGG CGGTTTGCGT ATTGGGCGCT CTTCCGCTTC | 3480 |
| | CTCGCTCACT GACTCGCTGC GCTCGGTCGT TCGGCTGCGG CGAGCGGTAT CAGCTCACTC | 3540 |
| | AAAGGCGGTA ATACGGTTAT CCACAGAATC AGGGGATAAC GCAGGAAAGA ACATGTGAGC | 3600 |
| | AAAAGGCCAG CAAAAGGCCA GGAACCGTAA AAAGGCCGCG TTGCTGGCGT TTTTCCATAG | 3660 |
| | GCTCCGCCCC CCTGACGAGC ATCACAAAAA TCGACGCTCA AGTCAGAGGT GGCGAAACCC | 3720 |
| | GACAGGACTA TAAAGATACC AGGCGTTTCC CCCTGGAAGC TCCCTCGTGC GCTCTCCTGT | 3780 |
| | TCCGACCCTG CCGCTTACCG GATACCTGTC CGCCTTTCTC CCTTCGGGAA GCGTGGCGCT | 3840 |
| | TTCTCATAGC TCACGCTGTA GGTATCTCAG TTCGGTGTAG GTCGTTCGCT CCAAGCTGGG | 3900 |
| | CTGTGTGCAC GAACCCCCCG TTCAGCCCGA CCGCTGCGCC TTATCCGGTA ACTATCGTCT | 3960 |
| | TGAGTCCAAC CCGGTAAGAC ACGACTTATC GCCACTGGCA GCAGCCACTG GTAACAGGAT | 4020 |
| | TAGCAGAGCG AGGTATGTAG GCGGTGCTAC AGAGTTCTTG AAGTGGTGGC CTAACTACGG | 4080 |
| | CTACACTAGA AGAACAGTAT TTGGTATCTG CGCTCTGCTG AAGCCAGTTA CCTTCGGAAA | 4140 |
| | AAGAGTTGGT AGCTCTTGAT CCGGCAAACA AACCACCGCT GGTAGCGGTG GTTTTTTTGT | 4200 |
| | TTGCAAGCAG CAGATTACGC GCAGAAAAAA AGGATCTCAA GAAGATCCTT TGATCTTTTC | 4260 |
| | TACGGGGTCT GACGCTCAGT GGAACGAAAA CTCACGTTAA GGGATTTTGG TCATGAGATT | 4320 |
| | ATCAAAAAGG ATCTTCACCT AGATCCTTTT AAATTAAAAA TGAAGTTTTA AATCAATCTA | 4380 |
| | AAGTATATAT GAGTAAACTT GGTCTGACAG TTACCAATGC TTAATCAGTG AGGCACCTAT | 4440 |
| | CTCAGCGATC TGTCTATTTC GTTCATCCAT AGTTGCCTGA CTCCCCGTCG TGTAGATAAC | 4500 |
| | TACGATACGG GAGGGCTTAC CATCTGGCCC CAGTGCTGCA ATGATACCGC GAGACCCACG | 4560 |
| | CTCACCGGCT CCAGATTTAT CAGCAATAAA CCAGCCAGCC GGAAGGGCCG AGCGCAGAAG | 4620 |
| | TGGTCCTGCA ACTTTATCCG CCTCCATCCA GTCTATTAAT TGTTGCCGGG AAGCTAGAGT | 4680 |
| | AAGTAGTTCG CCAGTTAATA GTTTGCGCAA CGTTGTTGCC ATTGCTACAG GCATCGTGGT | 4740 |
| | GTCACGCTCG TCGTTTGGTA TGGCTTCATT CAGCTCCGGT TCCCAACGAT CAAGGCGAGT | 4800 |
| | TACATGATCC CCCATGTTGT GCAAAAAAGC GGTTAGCTCC TTCGGTCCTC CGATCGTTGT | 4860 |
| | CAGAAGTAAG TTGGCCGCAG TGTTATCACT CATGGTTATG GCAGCACTGC ATAATTCTCT | 4920 |
| | TACTGTCATG CCATCCGTAA GATGCTTTTC TGTGACTGGT GAGTACTCAA CCAAGTCATT | 4980 |
| | CTGAGAATAG TGTATGCGGC GACCGAGTTG CTCTTGCCCG GCGTCAATAC GGGATAATAC | 5040 |
| | CGCGCCACAT AGCAGAACTT TAAAAGTGCT CATCATTGGA AAACGTTCTT CGGGGCGAAA | 5100 |
| | ACTCTCAAGG ATCTTACCGC TGTTGAGATC CAGTTCGATG TAACCCACTC GTGCACCCAA | 5160 |
| | CTGATCTTCA GCATCTTTTA CTTTCACCAG CGTTTCTGGG TGAGCAAAAA CAGGAAGGCA | 5220 |
| | AAATGCCGCA AAAAAGGGAA TAAGGGCGAC ACGGAAATGT TGAATACTCA TACTCTTCCT | 5280 |
| | TTTTCAATAT TATTGAAGCA TTTATCAGGG TTATTGTCTC ATGAGCGGAT ACATATTTGA | 5340 |
| | ATGTATTTAG AAAAATAAAC AAATAGGGGT TCCGCGCACA TTTCCCCGAA AAGTGCCACC | 5400 |
| | TGACGTCTAA GAAACCATTA TTATCATGAC ATTAACCTAT AAAAATAGGC GTATCACGAG | 5460 |
| | GCCCTTTCGT CTCGCGCGTT TCGGTGATGA CGGTGAAAAC CTCTGACACA TGCAGCTCCC | 5520 |
| | GGAGACGGTC ACAGCTTGTC TGTAAGCGGA TGCCGGGAGC AGACAAGCCC GTCAGGGCGC | 5580 |
| | GTCAGCGGGT GTTGGCGGGT GTCGGGGCTG GCTTAACTAT GCGGCATCAG AGCAGATTGT | 5640 |
| | ACTGAGAGTG CACCATATGC GGTGTGAAAT ACCGCACAGA TGCGTAAGGA GAAAATACCG | 5700 |
| | CATCAGGCGC CATTCGCCAT TCAGGCTGCG CAACTGTTGG GAAGGGCGAT CGGTGCGGGC | 5760 |
| | CTCTTCGCTA TTACGCCAGC TGGCGAAAGG GGGATGTGCT GCAAGGCGAT TAAGTTGGGT | 5820 |
| | AACGCCAGGG TTTTCCCAGT CACGACGTTG TAAAACGACG GCGCAAGGAA TGGTGCATGC | 5880 |
| | AAGGAGATGG CGCCCAACAG TCCCCCGGCC ACGGGGCCTG CCACCATACC CACGCCGAAA | 5940 |
| | CAAGCGCTCA TGAGCCCGAA GTGGCGAGCC CGATCTTCCC CATCGGTGAT GTCGGCGATA | 6000 |
| | TAGGCGCCAG CAACCGCACC TGTGGCGCCG GTGATGCCGG CCACGATGCG TCCGGCGTAG | 6060 |
| | AGGCGATTAG TCCAATTTGT TAAAGACAGG ATATCAGTGG TCCAGGCTCT AGTTTTGACT | 6120 |
| | CAACAATATC ACCAGCTGAA GCCTATAGAG TACGAGCCAT AGATAAAATA AAAGATTTTA | 6180 |
| | TTTAGTCTCC AGAAAAAGGG GGG | 6203 |
| SEQ ID NO: 670 | AATGAAAGAC CCCACCTGTA GGTTTGGCAA GCTAGCTTAA GTAACGCCAT TTTGCAAGGC | 60 |
| MSCV CCR8 | ATGGAAAATA CATAACTGAG AATAGAGAAG TTCAGATCAA GGTTAGGAAC AGAGAGACAG | 120 |
| retroviral | CAGAATATGG GCCAAACAGG ATATCTGTGG TAAGCAGTTC CTGCCCCGGC TCAGGGCCAA | 180 |
| vector | GAACAGATGG TCCCCAGATG CGGTCCCGCC CTCAGCAGTT TCTAGAGAAC CATCAGATGT | 240 |
| | TTCCAGGGTG CCCCAAGGAC CTGAAATGAC CCTGTGCCTT ATTTGAACTA ACCAATCAGT | 300 |
| | TCGCTTCTCG CTTCTGTTCG CGCGCTTCTG CTCCCCGAGC TCAATAAAAG AGCCCACAAC | 360 |
| | CCCTCACTCG GCGCGCCAGT CCTCCGATAG ACTGCGTCGC CCGGGTACCC GTATTCCCAA | 420 |
| | TAAAGCCTCT TGCTGTTTGC ATCCGAATCG TGGACTCGCT GATCCTTGGG AGGGTCTCCT | 480 |
| | CAGATTGATT GACTGCCCAC CTCGGGGGTC TTTCATTTGG AGGTTCCACC GAGATTTGGA | 540 |
| | GACCCCTGCC CAGGGACCAC CGACCCCCCC GCCGGGAGGT AAGCTGGCCA GCGGTCGTTT | 600 |
| | CGTGTCTGTC TCTGTCTTTG TGCGTGTTTG TGCCGGCATC TAATGTTTGC GCCTGCGTCT | 660 |
| | GTACTAGTTA GCTAACTAGC TCTGTATCTG GCGGACCCGT GGTGGAACTG ACGAGTTCTG | 720 |
| | AACACCCGGC CGCAACCCTG GGAGACGTCC CAGGGACTTT GGGGGCCGTT TTTGTGGCCC | 780 |
| | GACCTGAGGA AGGGAGTCGA TGTGGAATCC GACCCCGTCA GGATATGTGG TTCTGGTAGG | 840 |
| | AGACGAGAAC CTAAAACAGT TCCCGCCTCC GTCTGAATTT TTGCTTTCGG TTTGGAACCG | 900 |
| | AAGCCGCGCG TCTTGTCTGC TGCAGCGCTG CAGCATCGTT CTGTGTTGTC TCTGTCTGAC | 960 |
| | TGTGTTTCTG TATTTGTCTG AAAATTAGGG CCAGACTGTT ACCACTCCCT TAAGTTTGAC | 1020 |
| | CTTAGGTCAC TGGAAAGATG TCGAGCGGAT CGCTCACAAC CAGTCGGTAG ATGTCAAGAA | 1080 |
| | GAGACGTTGG GTTACCTTCT GCTCTGCAGA ATGGCCAACC TTTAACGTCG GATGGCCGCG | 1140 |
| | AGACGGCACC TTTAACCGAG ACCTCATCAC CCAGGTTAAG ATCAAGGTCT TTTCACCTGG | 1200 |
| | CCCGCATGGA CACCCAGACC AGGTCCCCTA CATCGTGACC TGGGAAGCCT TGGCTTTTGA | 1260 |
| | CCCCCCTCCC TGGGTCAAGC CCTTTGTACA CCCTAAGCCT CCGCCTCCTC TTCCTCCATC | 1320 |
| | CGCCCCGTCT CTCCCCCTTG AACCTCCTCG TTCGACCCCG CCTCGATCCT CCCTTTATCC | 1380 |
| | AGCCCTCACT CCTTCTCTAG GCGCCGGAAT TCCAAGTTTG TACAAAAAAG CAGGCTGCCA | 1440 |
| | CCATGGATTA TACACTTGAC CTCAGTGTGA CAACAGTGAC CGACTACTAC TACCCTGATA | 1500 |
| | TCTTCTCAAG CCCCTGTGAT GCGGAACTTA TTCAGACAAA TGGCAAGTTG CTCCTTGCTG | 1560 |
| | TCTTTTATTG CCTCCTGTTT GTATTCAGTC TTCTGGGAAA CAGCCTGGTC ATCCTGGTCC | 1620 |
| | TTGTGGTCTG CAAGAAGCTG AGGAGCATCA CAGATGTATA CCTCTTGAAC CTGGCCCTGT | 1680 |
| | CTGACCTGCT TTTTGTCTTC TCCTTCCCCT TTCAGACCTA CTATCTGCTG GACCAGTGGG | 1740 |
| | TGTTTGGGAC TGTAATGTGC AAAGTGGTGT CTGGCTTTTA TTACATTGGC TTCTACAGCA | 1800 |
| | GCATGTTTTT CATCACCCTC ATGAGTGTGG ACAGGTACCT GGCTGTTGTC CATGCCGTGT | 1860 |
| | ATGCCCTAAA GGTGAGGACG ATCAGGATGG GCACAACGCT GTGCCTGGCA GTATGGCTAA | 1920 |
| | CCGCCATTAT GGCTACCATC CCATTGCTAG TGTTTTACCA AGTGGCCTCT GAAGATGGTG | 1980 |
| | TTCTACAGTG TTATTCATTT TACAATCAAC AGACTTTGAA GTGGAAGATC TTCACCAACT | 2040 |
| | TCAAAATGAA CATTTTAGGC TTGTTGATCC CATTCACCAT CTTTATGTTC TGCTACATTA | 2100 |
| | AAATCCTGCA CCAGCTGAAG AGGTGTCAAA ACCACAACAA GACCAAGGCC ATCAGGTTGG | 2160 |
| | TGCTCATTGT GGTCATTGCA TCTTTACTTT TCTGGGTCCC ATTCAACGTG GTTCTTTTCC | 2220 |
| | TCACTTCCTT GCACAGTATG CACATCTTGG ATGGATGTAG CATAAGCCAA CAGCTGACTT | 2280 |
| | ATGCCACCCA TGTCACAGAA ATCATTTCCT TTACTCACTG CTGTGTGAAC CCTGTTATCT | 2340 |
| | ATGCTTTTGT TGGGGAGAAG TTCAAGAAAC ACCTCTCAGA AATATTTCAG AAAAGTTGCA | 2400 |
| | GCCAAATCTT CAACTACCTA GGAAGACAAA TGCCTAGGGA GAGCTGTGAA AAGTCATCAT | 2460 |
| | CCTGCCAGCA GCACTCCTCC CGTTCCTCCA GCGTAGACTA CATTTTGTGA ACCCAGCTTT | 2520 |
| | CTTGTACAAA GTGGCTCGAG AGATCCGTCG ACCTGCAGCC AAGCTTATCG ATAAAATAAA | 2580 |
| | AGATTTTATT TAGTCTCCAG AAAAAGGGGG GAATGAAAGA CCCCACCTGT AGGTTTGGCA | 2640 |
| | AGCTAGCTTA AGTAACGCCA TTTTGCAAGG CATGGAAAAT ACATAACTGA GAATAGAGAA | 2700 |
| | GTTCAGATCA AGGTTAGGAA CAGAGAGACA GCAGAATATG GGCCAAACAG GATATCTGTG | 2760 |
| | GTAAGCAGTT CCTGCCCCGG CTCAGGGCCA AGAACAGATG GTCCCCAGAT GCGGTCCCGC | 2820 |
| | CCTCAGCAGT TTCTAGAGAA CCATCAGATG TTTCCAGGGT GCCCCAAGGA CCTGAAATGA | 2880 |
| | CCCTGTGCCT TATTTGAACT AACCAATCAG TTCGCTTCTC GCTTCTGTTC GCGCGCTTCT | 2940 |
| | GCTCCCCGAG CTCAATAAAA GAGCCCACAA CCCCTCACTC GGCGCGCCAG TCCTCCGATA | 3000 |
| | GACTGCGTCG CCCGGGTACC CGTGTATCCA ATAAACCCTC TTGCAGTTGC ATCCGACTTG | 3060 |
| | TGGTCTCGCT GTTCCTTGGG AGGGTCTCCT CTGAGTGATT GACTACCCGT CAGCGGGGGT | 3120 |
| | CTTTCATGGG TAACAGTTTC TTGAAGTTGG AGAACAACAT TCTGAGGGTA GGAGTCGAAT | 3180 |
| | ATTAAGTAAT CCTGACTCAA TTAGCCACTG TTTTGAATCC ACATACTCCA ATACTCCTGA | 3240 |
| | AATAGTTCAT TATGGACAGC GCAGAAAGAG CTGGGGAGAA TTGTGAAATT GTTATCCGCT | 3300 |
| | CACAATTCCA CACAACATAC GAGCCGGAAG CATAAAGTGT AAAGCCTGGG GTGCCTAATG | 3360 |
| | AGTGAGCTAA CTCACATTAA TTGCGTTGCG CTCACTGCCC GCTTTCCAGT CGGGAAACCT | 3420 |
| | GTCGTGCCAG CTGCATTAAT GAATCGGCCA ACGCGCGGGG AGAGGCGGTT TGCGTATTGG | 3480 |
| | GCGCTCTTCC GCTTCCTCGC TCACTGACTC GCTGCGCTCG GTCGTTCGGC TGCGGCGAGC | 3540 |
| | GGTATCAGCT CACTCAAAGG CGGTAATACG GTTATCCACA GAATCAGGGG ATAACGCAGG | 3600 |
| | AAAGAACATG TGAGCAAAAG GCCAGCAAAA GGCCAGGAAC CGTAAAAAGG CCGCGTTGCT | 3660 |
| | GGCGTTTTTC CATAGGCTCC GCCCCCCTGA CGAGCATCAC AAAAATCGAC GCTCAAGTCA | 3720 |
| | GAGGTGGCGA AACCCGACAG GACTATAAAG ATACCAGGCG TTTCCCCCTG GAAGCTCCCT | 3780 |
| | CGTGCGCTCT CCTGTTCCGA CCCTGCCGCT TACCGGATAC CTGTCCGCCT TTCTCCCTTC | 3840 |
| | GGGAAGCGTG GCGCTTTCTC ATAGCTCACG CTGTAGGTAT CTCAGTTCGG TGTAGGTCGT | 3900 |
| | TCGCTCCAAG CTGGGCTGTG TGCACGAACC CCCCGTTCAG CCCGACCGCT GCGCCTTATC | 3960 |
| | CGGTAACTAT CGTCTTGAGT CCAACCCGGT AAGACACGAC TTATCGCCAC TGGCAGCAGC | 4020 |
| | CACTGGTAAC AGGASTAGCA GAGCGAGGTA TGTAGGCGGT GCTACAGAGT TCTTGAAGTG | 4080 |
| | GTGGCCTAAC TACGGCTACA CTAGAAGAAC AGTATTTGGT ATCTGCGCTC TGCTGAAGCC | 4140 |
| | AGTTACCTTC GGAAAAAGAG TTGGTAGCTC TTGATCCGGC AAACAAACCA CCGCTGGTAG | 4200 |
| | CGGTGGTTTT TTTGTTTGCA AGCAGCAGAT TACGCGCAGA AAAAAAGGAT CTCAAGAAGA | 4260 |
| | TCCTTTGATC TTTTCTACGG GGTCTGACGC TCAGTGGAAC GAAAACTCAC GTTAAGGGAT | 4320 |
| | TTTGGTCATG AGATTATCAA AAAGGATCTT CACCTAGATC CTTTTAAATT AAAAATGAAG | 4380 |
| | TTTTAAATCA ATCTAAAGTA TATATGAGTA AACTTGGTCT GACAGTTACC AATGCTTAAT | 4440 |
| | CAGTGAGGCA CCTATCTCAG CGATCTGTCT ATTTCGTTCA TCCATAGTTG CCTGACTCCC | 4500 |
| | CGTCGTGTAG ATAACTACGA TACGGGAGGG CTTACCATCT GGCCCCAGTG CTGCAATGAT | 4560 |
| | ACCGCGAGAC CCACGCTCAC CGGCTCCAGA TTTATCAGCA ATAAACCAGC CAGCCGGAAG | 4620 |
| | GGCCGAGCGC AGAAGTGGTC CTGCAACTTT ATCCGCCTCC ATCCAGTCTA TTAATTGTTG | 4680 |
| | CCGGGAAGCT AGAGTAAGTA GTTCGCCAGT TAATAGTTTG CGCAACGTTG TTGCCATTGC | 4740 |
| | TACAGGCATC GTGGTGTCAC GCTCGTCGTT TGGTATGGCT TCATTCAGCT CCGGTTCCCA | 4800 |
| | ACGATCAAGG CGAGTTACAT GATCCCCCAT GTTGTGCAAA AAAGCGGTTA GCTCCTTCGG | 4860 |
| | TCCTCCGATC GTTGTCAGAA GTAAGTTGGC CGCAGTGTTA TCACTCATGG TTATGGCAGC | 4920 |
| | ACTGCATAAT TCTCTTACTG TCATGCCATC CGTAAGATGC TTTTCTGTGA CTGGTGAGTA | 4980 |
| | CTCAACCAAG TCATTCTGAG AATAGTGTAT GCGGCGACCG AGTTGCTCTT GCCCGGCGTC | 5040 |
| | AATACGGGAT AATACCGCGC CACATAGCAG AACTTTAAAA GTGCTCATCA TTGGAAAACG | 5100 |
| | TTCTTCGGGG CGAAAACTCT CAAGGATCTT ACCGCTGTTG AGATCCAGTT CGATGTAACC | 5160 |
| | CACTCGTGCA CCCAACTGAT CTTCAGCATC TTTTACTTTC ACCAGCGTTT CTGGGTGAGC | 5220 |
| | AAAAACAGGA AGGCAAAATG CCGCAAAAAA GGGAATAAGG GCGACACGGA AATGTTGAAT | 5280 |
| | ACTCATACTC TTCCCTTTTC AATATTATTG AAGCATTTAT CAGGGTTATT GTCTCATGAG | 5340 |
| | CGGATACATA TTTGAATGTA TTTAGAAAAA TAAACAAATA GGGGTTCCGC GCACATTTCC | 5400 |
| | CCGAAAAGTG CCACCTGACG TCTAAGAAAC CATTATTATC ATGACATTAA CCTATAAAAA | 5460 |
| | TAGGCGTATC ACGAGGCCCT TTCGTCTCGC GCGTTTCGGT GATGACGGTG AAAACCTCTG | 5520 |
| | ACACATGCAG CTCCCGGAGA CGGTCACAGC TTGTCTGTAA GCGGATGCCG GGAGCAGACA | 5580 |
| | AGCCCGTCAG GGCGCGTCAG CGGGTGTTGG CGGGTGTCGG GGCTGGCTTA ACTATGCGGC | 5640 |
| | ATCAGAGCAG ATTGTACTGA GAGTGCACCA TATGCGGTGT GAAATACCGC ACAGATGCGT | 5700 |
| | AAGGAGAAAA TACCGCATCA GGCGCCATTC GCCATTCAGG CTGCGCAACT GTTGGGAAGG | 5760 |
| | GCGATCGGTG CGGGCCTCTT CGCTATTACG CCAGCTGGCG AAAGGGGGAT GTGCTGCAAG | 5820 |
| | GCGATTAAGT TGGGTAACGC CAGGGTTTTC CCAGTCACGA CGTTGTAAAA CGACGGCGCA | 5880 |
| | AGGAATGGTG CATGCAAGGA GATGGCGCCC AACAGTCCCC CGGCCACGGG GCCTGCCACC | 5940 |
| | ATACCCACGC CGAAACAAGC GCTCATGAGC CCGAAGTGGC GAGCCCGATC TTCCCCATCG | 6000 |
| | GTGATGTCGG CGATATAGGC GCCAGCAACC GCACCTGTGG CGCCGGTGAT GCCGGCCACG | 6060 |
| | ATGCGTCCGG CGTAGAGGCG ATTAGTCCAA TTTGTTAAAG ACAGGATATC AGTGGTCCAG | 6120 |
| | GCTCTAGTTT TGACTCAACA ATATCACCAG CTGAAGCCTA TAGAGTACGA GCCATAGATA | 6180 |
| | AAATAAAAGA TTTTATTTAG TCTCCAGAAA AAGGGGGG | 6218 |

In an embodiment, a chemokine receptor is encoded by a nucleotide sequence comprising SEQ ID NO:669. In an embodiment, a chemokine receptor is encoded by a nucleotide sequence comprising a region that is at least 99% identical to SEQ ID NO:669. In an embodiment, a chemokine receptor is encoded by a nucleotide sequence comprising a region that is at least 98% identical to SEQ ID NO:669. In an embodiment, a chemokine receptor is encoded by a nucleotide sequence comprising a region that is at least 97% identical to SEQ ID NO:669. In an embodiment, a chemokine receptor is encoded by a nucleotide sequence comprising a region that is at least 96% identical to SEQ ID NO:669. In an embodiment, a chemokine receptor is encoded by a nucleotide sequence comprising a region that is at least 95% identical to SEQ ID NO:669. In an embodiment, a chemokine receptor is encoded by a nucleotide sequence comprising a region that is at least 94% identical to SEQ ID NO:669. In an embodiment, a chemokine receptor is encoded by a nucleotide sequence comprising a region that is at least 93% identical to SEQ ID NO:669. In an embodiment, a chemokine receptor is encoded by a nucleotide sequence comprising a region that is at least 92% identical to SEQ ID NO:669. In an embodiment, a chemokine receptor is encoded by a nucleotide sequence comprising a region that is at least 91% identical to SEQ ID NO:669. In an embodiment, a chemokine receptor is encoded by a nucleotide sequence comprising a region that is at least 90% identical to SEQ ID NO:669. In an embodiment, a chemokine receptor is encoded by a nucleotide sequence comprising a region that is at least 85% identical to SEQ ID NO:669. In an embodiment, a chemokine receptor is encoded by a nucleotide sequence comprising a region that is at least 80% identical to SEQ ID NO:669.

In an embodiment, a chemokine receptor is encoded by a nucleotide sequence comprising SEQ ID NO:670. In an embodiment, a chemokine receptor is encoded by a nucleotide sequence comprising a region that is at least 99% identical to SEQ ID NO:670. In an embodiment, a chemokine receptor is encoded by a nucleotide sequence comprising a region that is at least 98% identical to SEQ ID NO:670. In an embodiment, a chemokine receptor is encoded by a nucleotide sequence comprising a region that is at least 97% identical to SEQ ID NO:670. In an embodiment, a chemokine receptor is encoded by a nucleotide sequence comprising a region that is at least 96% identical to SEQ ID NO:670. In an embodiment, a chemokine receptor is encoded by a nucleotide sequence comprising a region that is at least 95% identical to SEQ ID NO:670. In an embodiment, a chemokine receptor is encoded by a nucleotide sequence comprising a region that is at least 94% identical to SEQ ID NO:670. In an embodiment, a chemokine receptor is encoded by a nucleotide sequence comprising a region that is at least 93% identical to SEQ ID NO:670. In an embodiment, a chemokine receptor is encoded by a nucleotide sequence comprising a region that is at least 92% identical to SEQ ID NO:670. In an embodiment, a chemokine receptor is encoded by a nucleotide sequence comprising a region that is at least 91% identical to SEQ ID NO:670. In an embodiment, a chemokine receptor is encoded by a nucleotide sequence comprising a region that is at least 90% identical to SEQ ID NO:670. In an embodiment, a chemokine receptor is encoded by a nucleotide sequence comprising a region that is at least 85% identical to SEQ ID NO:670. In an embodiment, a chemokine receptor is encoded by a nucleotide sequence comprising a region that is at least 80% identical to SEQ ID NO:670.

In an embodiment, more than one chemokine receptor is encoded by multiple transgenes in a polycistronic vector. In an embodiment, at least one chemokine receptor and at least one CCR are encoded by multiple transgenes in a polycistronic vector. In an embodiment, at least two chemokine receptors and at least one CCR are encoded by multiple transgenes in a polycistronic vector. In an embodiment, at least one chemokine receptor and at least two CCRs are encoded by multiple transgenes in a polycistronic vector. In any of the foregoing embodiments, the CCRs and/or chemokine receptors are encoded by a bicistronic vector. Suitable polycistronic vectors are described herein and in Liu, et al., Scientific Reports 2017, 7(1), 2193, the disclosures of which are incorporated herein by reference. The IRES technique may also be employed in embodiments herein to achieve polycistronic vector designs.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Preparation of Media for Pre-Rep and Rep Processes

This example describes the procedure for the preparation of tissue culture media for use in protocols involving the culture of tumor infiltrating lymphocytes (TIL) derived from various solid tumors. This media can be used for preparation of any of the TILs described in the present application and other examples.

Preparation of CM1. Remove the following reagents from cold storage and warm them in a 37° C. water bath: (RPMI1640, Human AB serum, 200 mM L-glutamine). Prepare CM1 medium according to Table 67 below by adding each of the ingredients into the top section of a 0.2 μm filter unit appropriate to the volume to be filtered. Store at 4° C.

TABLE 67
Preparation of CM1.

| | Final | | Final | | Final | |
|---|---|---|---|---|---|---|
| Ingredient | concentration | | Volume 500 mL | | Volume IL | |
| RPMI1640 | NA | | 450 | mL | 900 | mL |
| Human AB serum, | 50 | mL | 100 | mL | | |
| heat-inactivated 10% | | | | | | |
| 200 mM L-glutamine | 2 | mM | 5 | mL | 10 | mL |
| 55 mM BME | 55 | μM | 0.5 | mL | 1 | mL |
| 50 mg/mL gentamicin | 50 | μg/mL | 0.5 | mL | 1 | mL |

sulfate

On the day of use, prewarm the required amount of CM1 in 37° C. water bath and add 6000 IU/mL IL-2.

Additional supplementation may be performed as needed according to Table 68.

TABLE 68
Additional supplementation of CM1, as needed.

| | Stock | | | Final | |
|---|---|---|---|---|---|
| Supplement | concentration | | Dilution | concentration | |
| GlutaMAX ™ | 200 | mM | 1:100 | 2 | mM |
| Penicillin/ | 10,000 U/mL | | 1:100 | 100 U/mL penicillin | |
| streptomycin | penicillin | | | 100 μg/mL | |
| | 10,000 μg/mL | | | streptomycin | |
| | streptomycin | | | | |
| Amphotericin B | 250 | μg/mL | 1:100 | 2.5 | μg/mL |

Preparation of CM2. Remove prepared CM1 from refrigerator or prepare fresh CM1 as per Section 7.3 above. Remove AIM-V® from refrigerator and prepare the amount of CM2 needed by mixing prepared CM1 with an equal volume of AIM-V® in a sterile media bottle. Add 3000 IU/mL IL-2 to CM2 medium on the day of usage. Make a sufficient amount of CM2 with 3000 IU/mL IL-2 on the day of usage. Label the CM2 media bottle and store at 4° C. until needed for tissue culture.

Preparation of CM3. Prepare CM3 on the day it was required for use. CM3 was the same as AIM-V® medium, supplemented with 3000 IU/mL IL-2 on the day of use. Prepare an amount of CM3 sufficient to experimental needs by adding IL-2 stock solution directly to the bottle or bag of AIM-V. Mix well by gentle shaking. Label bottle as “3000 IU/mL IL-2” immediately after adding to the AIM-V. If there was excess CM3, store it in labeled bottles at 4° C., with an expiration date of 7 days after preparation. Discard media supplemented with IL-2 after 7 days storage at 4° C.

Preparation of CM4. CM4 is the same as CM3, with the additional supplement of 2 mM GlutaMAX™ (final concentration). For every 1 L of CM3, add 10 mL of 200 mM GlutaMAX™. Prepare an amount of CM4 sufficient to experimental needs by adding IL-2 stock solution and GlutaMAX™ stock solution directly to the bottle or bag of AIM-V. Mix well by gentle shaking. Labeled bottle as “3000 IL/mL IL-2 and GlutaMAX” immediately after adding to the AIM-V. If there was excess CM4, store it in bottles at 4° C. labeled with the media name and its expiration date (7 days after preparation). Discard media supplemented with IL-2 after more than 7 days storage at 4° C.

### Example 2: Use of IL-2, IL-15, and IL-21 Cytokine Cocktail

This example describes the use of IL-2, IL-15, and IL-21 cytokines, which serve as additional T cell growth factors, in combination with the TIL process of any of the examples herein.

Using the processes described herein, TILs can be grown from tumors in presence of IL-2 in one arm of the experiment and, in place of IL-2, a combination of IL-2, IL-15, and IL-21 in another arm at the initiation of culture. At the completion of the pre-REP, cultures were assessed for expansion, phenotype, function (CD107a+ and IFN-γ) and TCR Vβ repertoire. IL-15 and IL-21 are described elsewhere herein and in Santegoets, et al., J. Transl. Med., 2013, 11, 37.

The results can show that enhanced TIL expansion (>20%), in both CD4⁺ and CD8⁺ cells in the IL-2, IL-15, and IL-21 treated conditions can observed relative to the IL-2 only conditions. There was a skewing towards a predominantly CD8⁺ population with a skewed TCR Vβ repertoire in the TILs obtained from the IL-2, IL-15, and IL-21 treated cultures relative to the IL-2 only cultures. IFN-γ and CD107a were elevated in the IL-2, IL-15, and IL-21 treated TILs, in comparison to TILs treated only IL-2.

### Example 3: Qualifying Individual Lots of Gamma-Irradiated Peripheral Mononuclear Cells

This Example describes an abbreviated procedure for qualifying individual lots of gamma-irradiated peripheral mononuclear cells (PBMCs, also known as mononuclear cells or MNCs) for use as allogeneic feeder cells in the exemplary methods described herein.

Each irradiated MNC feeder lot was prepared from an individual donor. Each lot or donor was screened individually for its ability to expand TIL in the REP in the presence of purified anti-CD3 (clone OKT3) antibody and interleukin-2 (IL-2). In addition, each lot of feeder cells was tested without the addition of TIL to verify that the received dose of gamma radiation was sufficient to render them replication incompetent.

Gamma-irradiated, growth-arrested MNC feeder cells are required for REP of TILs. Membrane receptors on the feeder MNCs bind to anti-CD3 (clone OKT3) antibody and crosslink to TILs in the REP flask, stimulating the TIL to expand. Feeder lots were prepared from the leukapheresis of whole blood taken from individual donors. The leukapheresis product was subjected to centrifugation over Ficoll-Hypaque, washed, irradiated, and cryopreserved under GMP conditions.

It is important that patients who received TIL therapy not be infused with viable feeder cells as this can result in graft-versus-host disease (GVHD). Feeder cells are therefore growth-arrested by dosing the cells with gamma-irradiation, resulting in double strand DNA breaks and the loss of cell viability of the MNC cells upon re-culture.

Feeder lots were evaluated on two criteria: (1) their ability to expand TILs in co-culture >100-fold and (2) their replication incompetency.

Feeder lots were tested in mini-REP format utilizing two primary pre-REP TIL lines grown in upright T25 tissue culture flasks. Feeder lots were tested against two distinct TIL lines, as each TIL line is unique in its ability to proliferate in response to activation in a REP. As a control, a lot of irradiated MNC feeder cells which has historically been shown to meet the criteria above was run alongside the test lots.

To ensure that all lots tested in a single experiment receive equivalent testing, sufficient stocks of the same pre-REP TIL lines were available to test all conditions and all feeder lots.

For each lot of feeder cells tested, there was a total of six T25 flasks: Pre-REP TIL line #1 (2 flasks); Pre-REP TIL line #2 (2 flasks); and feeder control (2 flasks). Flasks containing TIL lines #1 and #2 evaluated the ability of the feeder lot to expand TIL. The feeder control flasks evaluated the replication incompetence of the feeder lot.

### A. Experimental Protocol

Day −2/3, Thaw of TIL lines. Prepare CM2 medium and warm CM2 in 37° C. water bath. Prepare 40 mL of CM2 supplemented with 3000 IU/mL IL-2. Keep warm until use. Place 20 mL of pre-warmed CM2 without IL-2 into each of two 50 mL conical tubes labeled with names of the TIL lines used. Removed the two designated pre-REP TIL lines from LN2 storage and transferred the vials to the tissue culture room. Thawed vials by placing them inside a sealed zipper storage bag in a 37° C. water bath until a small amount of ice remains.

Using a sterile transfer pipet, the contents of each vial were immediately transferred into the 20 mL of CM2 in the prepared, labeled 50 mL conical tube. QS to 40 mL using CM2 without IL-2 to wash cells and centrifuged at 400×CF for 5 minutes. Aspirated the supernatant and resuspend in 5 mL warm CM2 supplemented with 3000 IU/mL IL-2.

A small aliquot (20 μL) was removed in duplicate for cell counting using an automated cell counter. The counts were recorded. While counting, the 50 mL conical tube with TIL cells was placed into a humidified 37° C., 5% CO₂ incubator, with the cap loosened to allow for gas exchange. The cell concentration was determined, and the TILs were diluted to 1×10⁶ cells/mL in CM2 supplemented with IL-2 at 3000 IU/mL.

Cultured in 2 mL/well of a 24-well tissue culture plate in as many wells as needed in a humidified 37° C. incubator until Day 0 of the mini-REP. The different TIL lines were cultured in separate 24-well tissue culture plates to avoid confusion and potential cross-contamination.

Day 0, initiate Mini-REP. Prepared enough CM2 medium for the number of feeder lots to be tested. (e.g., for testing 4 feeder lots at one time, prepared 800 mL of CM2 medium). Aliquoted a portion of the CM2 prepared above and supplemented it with 3000 IU/mL IL-2 for the culturing of the cells. (e.g., for testing 4 feeder lots at one time, prepare 500 mL of CM2 medium with 3000 IU/mL IL-2).

Working with each TIL line separately to prevent cross-contamination, the 24-well plate with TIL culture was removed from the incubator and transferred to the BSC.

Using a sterile transfer pipet or 100-1000 μL pipettor and tip, about 1 mL of medium was removed from each well of TILs to be used and placed in an unused well of the 24-well tissue culture plate.

Using a fresh sterile transfer pipet or 100-1000 μL pipettor and tip, the remaining medium was mixed with TILs in wells to resuspend the cells and then transferred the cell suspension to a 50 mL conical tube labeled with the TIL lot name and recorded the volume.

Washed the wells with the reserved media and transferred that volume to the same 50 mL conical tube. Spun the cells at 400×CF to collect the cell pellet. Aspirated off the media supernatant and resuspend the cell pellet in 2-5 mL of CM2 medium containing 3000 IU/mL IL-2, volume to be used based on the number of wells harvested and the size of the pellet—volume should be sufficient to ensure a concentration of >1.3×10⁶ cells/mL.

Using a serological pipet, the cell suspension was mixed thoroughly and the volume was recorded. Removed 200 μL for a cell count using an automated cell counter. While counting, placed the 50 mL conical tube with TIL cells into a humidified, 5% CO₂, 37° C. incubator, with the cap loosened to allow gas exchange. Recorded the counts.

Removed the 50 mL conical tube containing the TIL cells from the incubator and resuspend them cells at a concentration of 1.3×10⁶ cells/mL in warm CM2 supplemented with 3000 IU/mL IL-2. Returned the 50 mL conical tube to the incubator with a loosened cap.

The steps above were repeated for the second TIL line.

Just prior to plating the TIL into the T25 flasks for the experiment, TILs were diluted 1:10 for a final concentration of 1.3×10⁵ cells/mL as per below.

Prepare MACS GMP CD3 pure (OKT3) working solution. Took out stock solution of OKT3 (1 mg/mL) from 4° C. refrigerator and placed in BSC. A final concentration of 30 ng/mL OKT3 was used in the media of the mini-REP.

600 ng of OKT3 were needed for 20 mL in each T25 flask of the experiment; this was the equivalent of 60 μL of a 10 μg/mL solution for each 20 mL, or 360 μL for all 6 flasks tested for each feeder lot.

For each feeder lot tested, made 400 μL of a 1:100 dilution of 1 mg/mL OKT3 for a working concentration of 10 μg/mL (e.g., for testing 4 feeder lots at one time, make 1600 μL of a 1:100 dilution of 1 mg/mL OKT3: 16 μL of 1 mg/mL OKT3+1.584 mL of CM2 medium with 3000 IU/mL IL-2.)

Prepare T25 flasks. Labeled each flask and filled flask with the CM2 medium prior to preparing the feeder cells. Placed flasks into 37° C. humidified 5% CO₂ incubator to keep media warm while waiting to add the remaining components. Once feeder cells were prepared, the components will be added to the CM2 in each flask.

Further information is provided in Table 69.

TABLE 69
Solution information.

| | | | Volume in | |
|---|---|---|---|---|
| | Volume in co- | | control (feeder | |
| Component | culture flasks | | only) flasks | |
| CM2 + 3000 IU/mL IL-2 | 18 | mL | 19 | mL |
| MNC: 1.3 × 10⁷/mL in CM2 + 3000 | 1 | mL | 1 | mL |
| IU IL-2 | | | | |
| (final concentration 1.3 × 10⁷/flask) | | | | |
| OKT3: 10 μL/mL in CM2 = 3000 IU | 60 | μL | 60 | μL |

| IL-2 | | | |
|---|---|---|---|
| TIL: 1.3 × 10⁵/mL in CM2 with 3000 | 1 | mL | 0 |

IU of IL-2
(final concentration 1.3 × 10⁵/flask)

Prepare Feeder Cells. A minimum of 78×10⁶ feeder cells were needed per lot tested for this protocol. Each 1 mL vial frozen by SDBB had 100×10⁶ viable cells upon freezing. Assuming a 50% recovery upon thaw from liquid N2 storage, it was recommended to thaw at least two 1 mL vials of feeder cells per lot giving an estimated 100×10⁶ viable cells for each REP. Alternately, if supplied in 1.8 mL vials, only one vial provided enough feeder cells.

Before thawing feeder cells, approximately 50 mL of CM2 without IL-2 was pre-warmed for each feeder lot to be tested. The designated feeder lot vials were removed from LN2 storage, placed in zipper storage bag, and placed on ice. Vials were thawed inside closed zipper storage bag by immersing in a 37° C. water bath. Vials were removed from zipper bag, sprayed or wiped with 70% EtOH, and transferred to a BSC.

Using a transfer pipet, the contents of feeder vials were immediately transferred into 30 mL of warm CM2 in a 50 mL conical tube. The vial was washed with a small volume of CM2 to remove any residual cells in the vial, and centrifuged at 400×CF for 5 minutes. Aspirated the supernatant and resuspended in 4 mL warm CM2 plus 3000 IU/mL IL-2. Removed 200 μL for cell counting using the automated cell counter. Recorded the counts.

Resuspended cells at 1.3×10⁷ cells/mL in warm CM2 plus 3000 IU/mL IL-2. Diluted TIL cells from 1.3×10⁶ cells/mL to 1.3×10⁵ cells/mL.

Setup Co-Culture. Diluted TIL cells from 1.3×10⁶ cells/mL to 1.3×10⁵ cells/mL. Added 4.5 mL of CM2 medium to a 15 mL conical tube. Removed TIL cells from incubator and resuspended well using a 10 mL serological pipet. Removed 0.5 mL of cells from the 1.3×10⁶ cells/mL TIL suspension and added to the 4.5 mL of medium in the 15 mL conical tube. Returned TIL stock vial to incubator. Mixed well. Repeated for the second TIL line.

Transferred flasks with pre-warmed media for a single feeder lot from the incubator to the BSC. Mixed feeder cells by pipetting up and down several times with a 1 mL pipet tip and transferred 1 mL (1.3×10⁷ cells) to each flask for that feeder lot. Added 60 μL of OKT3 working stock (10 μg/mL) to each flask. Returned the two control flasks to the incubator.

Transferred 1 mL (1.3×10⁵) of each TIL lot to the correspondingly labeled T25 flask. Returned flasks to the incubator and incubate upright. Did not disturb until Day 5. This procedure was repeated for all feeder lots tested.

Day 5, Media change. Prepared CM2 with 3000 IU/mL IL-2. 10 mL is needed for each flask. With a 10 mL pipette, transferred 10 mL warm CM2 with 3000 IU/mL IL-2 to each flask. Returned flasks to the incubator and incubated upright until day 7. Repeated for all feeder lots tested.

Day 7, Harvest. Removed flasks from the incubator and transfer to the BSC, care as taken not to disturb the cell layer on the bottom of the flask. Without disturbing the cells growing on the bottom of the flasks, 10 mL of medium was removed from each test flask and 15 mL of medium from each of the control flasks.

Using a 10 mL serological pipet, the cells were resuspended in the remaining medium and mix well to break up any clumps of cells. After thoroughly mixing cell suspension by pipetting, removed 200 μL for cell counting. Counted the TIL using the appropriate standard operating procedure in conjunction with the automatic cell counter equipment. Recorded counts in day 7. This procedure was repeated for all feeder lots tested.

Feeder control flasks were evaluated for replication incompetence and flasks containing TILs were evaluated for fold expansion from day 0.

Day 7, Continuation of Feeder Control Flasks to Day 14. After completing the day 7 counts of the feeder control flasks, 15 mL of fresh CM2 medium containing 3000 IU/mL IL-2 was added to each of the control flasks. The control flasks were returned to the incubator and incubated in an upright position until day 14.

Day 14. Extended Non-proliferation of Feeder Control Flasks. Removed flasks from the incubator and transfer to the BSC, care was taken not to disturb the cell layer on the bottom of the flask. Without disturbing the cells growing on the bottom of the flasks, approximately 17 mL of medium was removed from each control flasks. Using a 5 mL serological pipet, the cells were resuspended in the remaining medium and mixed well to break up any clumps of cells. The volumes were recorded for each flask.

After thoroughly mixing the cell suspension by pipetting, 200 μL was removed for cell counting. The TILs were counted using the appropriate standard operating procedure in conjunction with the automatic cell counter equipment and the counts were recorded. This procedure was repeated for all feeder lots tested.

### B. Results and Acceptance Criteria Protocol

Results. The dose of gamma irradiation was sufficient to render the feeder cells replication incompetent. All lots were expected to meet the evaluation criteria and also demonstrated a reduction in the total viable number of feeder cells remaining on day 7 of the REP culture compared to day 0. All feeder lots were expected to meet the evaluation criteria of 100-fold expansion of TIL growth by day 7 of the REP culture. Day 14 counts of feeder control flasks were expected to continue the non-proliferative trend seen on day 7.

Acceptance Criteria. The following acceptance criteria were met for each replicate TIL line tested for each lot of feeder cells. Acceptance was two-fold, as follows in Table 70 below.

TABLE 70
Embodiments of acceptance criteria.

| Test | Acceptance criteria |
|---|---|
| Irradiation of MNC and | No growth observed at 7 and 14 days |
| Replication Incompetence | |
| TIL expansion | At least a 100-fold expansion of each |
| | TIL (minimum of 1.3 × 10⁷ viable |
| | cells) |

The dose of radiation was evaluated for its sufficiency to render the MNC feeder cells replication incompetent when cultured in the presence of 30 ng/mL OKT3 antibody and 3000 IU/mL IL-2. Replication incompetence was evaluated by total viable cell count (TVC) as determined by automated cell counting on day 7 and day 14 of the REP.

The acceptance criteria was “No Growth,” meaning the total viable cell number has not increased on day 7 and day 14 from the initial viable cell number put into culture on Day 0 of the REP.

The ability of the feeder cells to support TIL expansion was evaluated. TIL growth was measured in terms of fold expansion of viable cells from the onset of culture on day 0 of the REP to day 7 of the REP. On day 7, TIL cultures achieved a minimum of 100-fold expansion, (i.e., greater than 100 times the number of total viable TIL cells put into culture on REP day 0), as evaluated by automated cell counting.

Contingency Testing of MNC Feeder Lots that do not meet acceptance criteria. In the event that an MNC feeder lot did not meet the either of the acceptance criteria outlined above, the following steps will be taken to retest the lot to rule out simple experimenter error as its cause.

If there are two or more remaining satellite testing vials of the lot, then the lot was retested. If there were one or no remaining satellite testing vials of the lot, then the lot was failed according to the acceptance criteria listed above.

In order to be qualified, the lot in question and the control lot had to achieve the acceptance criteria above. Upon meeting these criteria, the lot is released for use.

### Example 4: Preparation of IL-2 Stock Solution

This Example describes the process of dissolving purified, lyophilized recombinant human interleukin-2 into stock samples suitable for use in further tissue culture protocols, including all of those described in the present application and Examples, including those that involve using rhIL-2.

Procedure. Prepare 0.2% Acetic Acid solution (HAc). Transfer 29 mL sterile water to a 50 mL conical tube. Add 1 mL 1 N acetic acid to the 50 mL conical tube. Mix well by inverting tube 2-3 times. Sterilize the HAc solution by filtration using a Steriflip filter.

Prepare 1% HSA in PBS. Add 4 mL of 25% HSA stock solution to 96 mL PBS in a 150 mL sterile filter unit. Filter solution and store at 4° C. For each vial of rhIL-2 prepared, fill out forms.

Prepare rhIL-2 stock solution (6×10⁶ IU/mL final concentration). Each lot of rhIL-2 was different and required information found in the manufacturer's Certificate of Analysis (COA), such as: 1) Mass of rhIL-2 per vial (mg), 2) Specific activity of rhIL-2 (IU/mg) and 3) Recommended 0.2% HAc reconstitution volume (mL).

The volume of 1% HSA required for rhIL-2 lot may be calculated by using the equation below:

$$( \frac{Vial Mass ( mg ) \times Biological Activity ( \frac{IU}{mg} )}{6 \times {10}^{6} \frac{IU}{mL}} ) − HAc vol ( mL ) = 1 \% HSA vol ( mL )$$

For example, according to CellGenix's rhIL-2 lot 10200121 COA, the specific activity for the 1 mg vial is 25×10⁶ IU/mg. It recommends reconstituting the rhIL-2 in 2 mL 0.2% HAc.

$$( \frac{1 mg \times 25 \times {10}^{6} \frac{IU}{mg}}{6 \times {10}^{6} \frac{IU}{mL}} ) − 2 mL = 2.167 mL HSA$$

Wiped rubber stopper of IL-2 vial with alcohol wipe. Using a 16G needle attached to a 3 mL syringe, injected recommended volume of 0.2% HAc into vial. Took care to not dislodge the stopper as the needle is withdrawn. Inverted vial 3 times and swirled until all powder is dissolved. Carefully removed the stopper and set aside on an alcohol wipe. Added the calculated volume of 1% HSA to the vial.

Storage of rhIL-2 solution. For short-term storage (<72 hours), stored vial at 4° C. For long-term storage (>72 hours), aliquoted vial into smaller volumes and stored in cryovials at −20° C. until ready to use. Avoided freeze/thaw cycles. Expired 6 months after date of preparation. Rh-IL-2 labels included vendor and catalog number, lot number, expiration date, operator initials, concentration and volume of aliquot.

### Example 5: Cryopreservation Process

This example describes a cryopreservation process method for TILs prepared with the procedures described herein using the CryoMed Controlled Rate Freezer, Model 7454 (Thermo Scientific).

The equipment used was as follows: aluminum cassette holder rack (compatible with CS750 freezer bags), cryostorage cassettes for 750 mL bags, low pressure (22 psi) liquid nitrogen tank, refrigerator, thermocouple sensor (ribbon type for bags), and CryoStore CS750 freezing bags (OriGen Scientific).

The freezing process provides for a 0.5° C. rate from nucleation to −20° C. and 1° C. per minute cooling rate to −80° C. end temperature. The program parameters are as follows: Step 1—wait at 4° C.; Step 2: 1.0° C./min (sample temperature) to −4° C.; Step 3: 20.0° C./min (chamber temperature) to −45° C.; Step 4: 10.0° C./min (chamber temperature) to −10.0° C.; Step 5: 0.5° C./min (chamber temperature) to −20° C.; and Step 6: 1.0° C./min (sample temperature) to −80° C.

### Example 6: Gen 2 and Gen 3 Exemplary Processes

This example demonstrates the Gen 2 and Gen 3 processes. Process Gen 2 and Gen 3 TILs are generally composed of autologous TIL derived from an individual patient through surgical resection of a tumor and then expanded ex vivo. The priming first expansion step of the Gen 3 process was a cell culture in the presence of interleukin-2 (IL-2) and the monoclonal antibody OKT3, which targets the T-cell co-receptor CD3 on a scaffold of irradiated peripheral blood mononuclear cells (PBMCs).

The manufacture of Gen 2 TIL products consists of two phases: 1) pre-Rapid Expansion (Pre-REP) and 2) Rapid Expansion Protocol (REP). During the Pre-REP resected tumors were cut up into ≤50 fragments 2-3 mm in each dimension which were cultured with serum-containing culture medium (RPMI 1640 media containing 10% HuSAB supplemented) and 6,000 IU/mL of Interleukin-2 (IL-2) for a period of 11 days. On day 11 TILs were harvested and introduced into the large-scale secondary REP expansion. The REP consists of activation of ≤200×10⁶ of the viable cells from pre-REP in a co-culture of 5×10⁹ irradiated allogeneic PBMCs feeder cells loaded with 150 μg of monoclonal anti-CD3 antibody (OKT3) in a 5 L volume of CM2 supplemented with 3000 IU/mL of rhIL-2 for 5 days. On day 16 the culture is volume reduced 90% and the cell fraction is split into multiple G-Rex-500 flasks at ≥1×10⁹ viable lymphocytes/flask and QS to 5 L with CM4. TILs are incubated for an additional 6 days. The REP is harvested on day 22, washed, formulated, and cryo-preserved prior to shipping at −150° C. to the clinical site for infusion.

The manufacture of Gen 3 TIL products consists of three phases: 1) Priming First Expansion Protocol, 2) Rapid Second Expansion Protocol (also referred to as rapid expansion phase or REP), and 3) Subculture Split. To effect the Priming First Expansion TIL propagation, resected tumor was cut up into ≤120 fragments 2-3 mm in each dimension. On day 0 of the Priming First Expansion, a feeder layer of approximately 2.5×10⁸ allogeneic irradiated PBMCs feeder cells loaded with OKT-3 was established on a surface area of approximately 100 cm² in each of 3 100 MCS vessels. The tumor fragments were distributed among and cultured in the 3 100 MCS vessels each with 500 mL serum-containing CM1 culture medium and 6,000 IU/mL of Interleukin-2 (IL-2) and 15 ug OKT-3 for a period of 7 days. On day 7, REP was initiated by incorporating an additional feeder cell layer of approximately 5×10⁸ allogeneic irradiated PBMCs feeder cells loaded with OKT-3 into the tumor fragmented culture phase in each of the three 100 MCS vessels and culturing with 500 mL CM2 culture medium and 6,000 IU/mL IL-2 and 30 μg OKT-3. The REP initiation was enhanced by activating the entire Priming First Expansion culture in the same vessel using closed system fluid transfer of OKT3 loaded feeder cells into the 100MCS vessel. For Gen 3, the TIL scale up or split involved process steps where the whole cell culture was scaled to a larger vessel through closed system fluid transfer and was transferred (from 100 M flask to a 500 M flask) and additional 4 L of CM4 media was added. The REP cells were harvested on day 16, washed, formulated, and cryo-preserved prior to shipping at −150° C. to the clinical site for infusion.

Overall, the Gen 3 process is a shorter, more scalable, and easily modifiable expansion platform, as summarized in Table 71.

TABLE 71
Comparison of Exemplary Gen 2 and Exemplary Gen 3 manufacturing processes.

| Step | Process (Gen 2) | Process (Gen 3) |
|---|---|---|
| Pre REP- | Up to 50 fragments, 1 G-Rex | Whole tumor up to 120 fragments |
| day 0 | 100MCS, 11 days | divided evenly among up to 3 flasks. 1 |
| | In 1 L of CM1 media + | flask: 1-60 fragments |
| | IL-2 (6000 IU/mL) | 2 flasks: 61-89 fragments |
| | | 3 flasks 90-120 fragments |
| | | 7 days in 500 mL of CMI media + |
| | | IL-2 (6000 IU/mL) |
| | | 2.5 × 10⁸ feeder cells/flask |
| | | 15 ug OKT-3/flask |
| REP | Direct to REP, Day 11, | Direct to REP, Day 7, all cells, same |
| Initiation | <200 × 10⁶ TIL | G-Rex 100MCS |
| | (1)G-Rex 500MCS in 5 L CM2 | Add 500 CM2 media |
| | media | IL-2 (6000 IU/mL) |
| | IL-2 (3000 IU/mL) | 5 × 10⁸ feeder cells/flask |
| | 5 × 10⁹ feeder cells | 30 ug OKT-3/flask |
| | 150 ug OKT-3 | |
| TIL | Volume reduce and split cell | Each G-Rex 100MCS (1 L) transfers |
| propagation | fraction in up to 5 G-Rex 500MCS | to 1 G-Rex 500MCS |
| or Scale up | 4.5 L CM4 media + IL-2 (3000 | Add 4 L CM4 media + IL-2 (3000 |
| | IU/mL) | IU/mL) |
| | ≥1 × 10⁹ TVC/flask | Scale up on day 9 to 11 |
| | Split day 16 | |
| Harvest | Harvest day 22, | Harvest day 16LOVO automated |
| | LOVO-automated cell washer | cell washer |
| Final | Cryopreserved Product | Cryopreserved product |
| formulation | 300 IU/mL IL2- CS10 in LN₂, | 300 IU/mL IL-2-CS10 in LN₂, |
| | multiple aliquots | multiple aliquots |
| Process | 22 days | 16 days |
| time | | |

On day 0, for both processes, the tumor was washed 3 times and the fragments were randomized and divided into two pools; one pool per process. For the Gen 2 Process, the fragments were transferred to one—GREX 100MCS flask with 1 L of CM1 media containing 6,000 IU/mL rhIL-2. For the Gen 3 Process, fragments were transferred to one G Rex 100MCS flask with 500 mL of CM1 containing 6,000 IU/mL rhIL-2, 15 ug OKT-3 and 2.5×10⁸ feeder cells. Seeding of TIL for Rep initiation day occurred on different days according to each process. For the Gen 2 Process, in which the G-Rex 100MCS flask was 90% volume reduced, collected cell suspension was transferred to a new G-Rex 500MCS to start REP initiation on day 11 in CM2 media containing IL-2 (3000 IU/mL), plus 5×10⁹ feeder cells and OKT-3 (30 ng/mL). Cells were expanded and split on day 16 into multiple G-Rex 500 MCS flasks with CM4 media with IL-2 (3000 IU/mL) per protocol. The culture was then harvested and cryopreserved on day 22 per protocol. For the Gen 3 process, the REP initiation occurred on day 7, in which the same G-Rex 100MCS used for REP initiation. Briefly, 500 mL of CM2 media containing IL-2 (6000 IU/mL) and 5×10⁸ feeder cells with 30 ug OKT-3 was added to each flask. On day 9-11 the culture was scaled up. The entire volume of the G-Rex100M (1 L) was transferred to a G-Rex 500MCS and 4 L of CM4 containing IL-2 (3000 IU/mL) was added. Flasks were incubated 5 days. Cultures were harvested and cryopreserved on Day 16.

Three different tumors were included in the comparison, two lung tumors (L4054 and L4055) and one melanoma tumor (M1085T).

CM1 (culture media 1), CM2 (culture media 2), and CM4 (culture media 4) media were prepared in advance and held at 4° C. for L4054 and L4055. CM1 and CM2 media were prepared without filtration to compare cell growth with and without filtration of media.

Media was warmed at 37° C. up to 24 hours in advance for L4055 tumor on REP initiation and scale-up.

Results. Gen 3 results fell within 30% of Gen 2 for total viable cells achieved. Gen 3 final product exhibited higher production of IFN-γ after restimulation. Gen 3 final product exhibited increased clonal diversity as measured by total unique CDR3 sequences present. Gen 3 final product exhibited longer mean telomere length.

Pre-REP and REP expansion on Gen 2 and Gen 3 processes followed the procedures described above. For each tumor, the two pools contained equal number of fragments. Due to the small size of tumors, the maximum number of fragments per flask was not achieved. Total pre-REP cells (TVC) were harvested and counted at day 11 for the Gen 2 process and at day 7 for the Gen 3 process. To compare the two pre-REP arms, the cell count was divided over the number of fragments provided in the culture in order to calculate an average of viable cells per fragment. As indicated in Table 72 below, the Gen 2 process consistently grew more cells per fragment compared to the Gen 3 Process. An extrapolated calculation of the number of TVC expected for Gen 3 process at day 11, which was calculated dividing the pre-REP TVC by 7 and then multiplying by 11.

TABLE 72
Pre-REP cell counts.

| | Tumor ID | | | | | |
|---|---|---|---|---|---|---|
| | L4054 | | L4055* | | M1085T | |
| Process | Gen 2 | Gen 3 | Gen 2 | Gen 3 | Gen 2 | Gen 3 |
| pre-REP TVC | 1.42E+08 | 4.32E+07 | 2.68E+07 | 1.38E+07 | 1.23E+07 | 3.50E+06 |
| Number of fragments | 21 | 21 | 24 | 24 | 16 | 16 |
| Average TVC per | 6.65E+06 | 2.06E+06 | 1.12E+06 | 5.75E+05 | 7.66E+05 | 2.18E+05 |
| fragment at pre-REP | | | | | | |
| Gen 3 extrapolated | N/A | 6.79E+07 | N/A | 2.17E+07 | N/A | 5.49E+06 |
| value at pre REP day 11 | | | | | | |

*L4055, unfiltered media.

For the Gen 2 and Gen 3 processes, TVC was counted per process condition and percent viable cells was generated for each day of the process. On harvest, day 22 (Gen 2) and day 16 (Gen 3) cells were collected and the TVC count was established. The TVC was then divided by the number of fragments provided on day 0, to calculate an average of viable cells per fragment. Fold expansion was calculated by dividing harvest TVC by over the REP initiation TVC. As exhibited in Table 73, comparing Gen 2 and the Gen 3 processes, fold expansions were similar for L4054; in the case of L4055, the fold expansion was higher for the Gen 2 process. Specifically, in this case, the media was warmed up 24 in advance of REP initiation day. A higher fold expansion was also observed in Gen 3 for M1085T. An extrapolated calculation of the number of TVC expected for Gen 3 process at day 22, which was calculated dividing the REP TVC by 16 and then multiply by 22.

TABLE 73
Total viable cell count and fold expansion on TIL final product.

| | Tumor ID | | | | | |
|---|---|---|---|---|---|---|
| | L4054 | | L4055 | | M1085T | |
| Process | Gen 2 | Gen 3 | Gen 2 | Gen 3 | Gen 2 | Gen 3 |
| # Fragments | 21 | 21 | 24 | 24 | 16 | 16 |
| TVC /fragment (at | 3.18E+09 | 8.77E+08 | 2.30E+09 | 3.65E+08 | 7.09E+08 | 4.80E+08 |
| Harvest) | | | | | | |
| REP initiation | 1.42E+08 | 4.32E+07 | 2.68E+07 | 1.38E+07 | 1.23E+07 | 3.50E+06 |
| Scale up | 3.36E+09 | 9.35E+08 | 3.49E+09 | 8.44E+08 | 1.99E+09 | 3.25E+08 |
| Harvest | 6.67E+10 | 1.84E+10 | 5.52E+10 | 8.76E+09 | 1.13E+10 | 7.68E+09 |
| Fold Expansion Harvest/ | 468.4 | 425.9 | 2056.8 | 634.6 | 925.0 | 2197.2 |
| REP initiation | | | | | | |
| Gen 3 extrapolated value | N/A | 2.53E+10 | N/A | 1.20E+10 | N/A | 1.06E+10 |
| at REP harvest day 22 | | | | | | |

* L4055, unfiltered media.

Upon harvest, the final TIL REP products were compared against release criteria for % viability, with results given in Table 74. All of the conditions for the Gen 2 and Gen 3 processes surpassed the 70% viability criterion and were comparable across processes and tumors.

TABLE 74
% Viability of REP (TIL Final Product).

| | Tumor ID | | | | | |
|---|---|---|---|---|---|---|
| | L4054 | | L4055 | | M1085T | |
| Process | Gen 2 | Gen 3 | Gen 2 | Gen 3 | Gen 2 | Gen 3 |
| REP initiation | 98.23% | 97.97% | 97.43% | 92.03% | 81.85% | 68.27% |
| Scale up | 94.00% | 93.57% | 90.50% | 95.93% | 78.55% | 71.15% |
| Harvest | 87.95% | 89.85% | 87.50% | 86.70% | 86.10% | 87.45% |

Due to the number of fragments per flask below the maximum required number, an estimated cell count at harvest day was calculated for each tumor, as shown in Table 75. The estimation was based on the expectation that clinical tumors were large enough to seed 2 or 3 flasks on day 0.

TABLE 75
Extrapolated estimate cell count calculation
to full scale 2 and 3 flask on Gen 3 process.

| | Tumor ID | | | | | |
|---|---|---|---|---|---|---|
| | L4054 | | L4055 | | M1085T | |
| Gen 3 Process | 2 flasks | 3 Flasks | 2 flasks | 3 Flasks | 2 flasks | 3 Flasks |
| Estimate Harvest | 3.68E+10 | 5.52E+10 | 1.75E+10 | 2.63E+10 | 1.54E+10 | 2.30E+10 |

Immunophenotyping—phenotypic marker comparisons on TIL final product. Three tumors L4054, L4055, and M1085T underwent TIL expansion in both the Gen 2 and Gen 3 processes. Upon harvest, the REP TIL final products were subjected to flow cytometry analysis to test purity, differentiation, and memory markers. For all the conditions the percentage of TCR a/b+ cells was over 90%.

TIL harvested from the Gen 3 process showed a higher expression of CD8 and CD28 compared to TIL harvested from the Gen 2 process. The Gen 2 process showed a higher percentage of CD4+.

TIL harvested from the Gen 3 process showed a higher expression on central memory compartments compared to TIL from the Gen 2 process.

Activation and exhaustion markers were analyzed in TIL from two, tumors L4054 and L4055 to compare the final TIL product by from the Gen 2 and Gen 3 TIL expansion processes. Activation and exhaustion markers were comparable between the Gen 2 and Gen 3 processes.

Interferon gamma secretion upon restimulation. On harvest day, day 22 for Gen 2 and day 16 for Gen 3, TIL underwent an overnight restimulation with coated anti-CD3 plates for L4054 and L4055. The restimulation on M1085T was performed using anti-CD3. CD28, and CD137 beads. Supernatant was collected after 24 hours of the restimulation in all conditions and the supernatant was frozen. IFNγ analysis by ELISA was assessed on the supernatant from both processes at the same time using the same ELISA plate. Higher production of IFNγ from the Gen 3 process was observed in the three tumors analyzed.

Measurement of IL-2 levels in culture media. To compare the IL-2 consumption between Gen 2 and Gen 3 process, cell supernatant was collected on REP initiation, scale up, and harvest day, on tumor L4054 and L4055. The quantity of IL-2 in cell culture supernatant was measured by Quantitate ELISA Kit from R&D. The general trend indicates that the IL-2 concentration remains higher in the Gen 3 process when compared to the Gen 2 process. This is likely due to the higher concentration of IL-2 on REP initiation (6000 IU/mL) for Gen 3 coupled with the carryover of the media throughout the process.

Metabolic substrate and metabolite analysis. The levels of metabolic substrates such as D-glucose and L-glutamine were measured as surrogates of overall media consumption. Their reciprocal metabolites, such lactic acid and ammonia, were measured. Glucose is a simple sugar in media that is utilized by mitochondria to produce energy in the form of ATP. When glucose is oxidized, lactic acid is produced (lactate is an ester of lactic acid). Lactate is strongly produced during the cells exponential growth phase. High levels of lactate have a negative impact on cell culture processes.

Spent media for L4054 and L4055 was collected at REP initiation, scale up, and harvest days for both process Gen 2 and Gen 3. The spent media collection was for Gen 2 on Day 11, day 16 and day 22; for Gen 3 was on day 7, day 11 and day 16. Supernatant was analyzed on a CEDEX Bio-analyzer for concentrations of glucose, lactic acid, glutamine, GlutaMax, and ammonia.

L-glutamine is an unstable essential amino acid required in cell culture media formulations. Glutamine contains an amine, and this amide structural group can transport and deliver nitrogen to cells. When L-glutamine oxidizes, a toxic ammonia by-product is produced by the cell. To counteract the degradation of L-glutamine the media for the Gen 2 and Gen 3 processes was supplemented with GlutaMax, which is more stable in aqueous solutions and does not spontaneously degrade. In the two tumor lines, the Gen 3 arm showed a decrease in L-glutamine and GlutaMax during the process and an increase in ammonia throughout the REP. In the Gen 2 arm a constant concentration of L-glutamine and GlutaMax, and a slight increase in the ammonia production was observed. The Gen 2 and Gen 3 processes were comparable at harvest day for ammonia and showed a slight difference in L-glutamine degradation.

Telomere repeats by Flow-FISH. Flow-FISH technology was used to measure the average length of the telomere repeat on L4054 and L4055 under Gen 2 and Gen 3 process. The determination of a relative telomere length (RTL) was calculated using Telomere PNA kit/FITC for flow cytometry analysis from DAKO. Gen 3 showed comparable telomere length to Gen 2.

CD3 Analysis. To determine the clonal diversity of the cell products generated in each process, TIL final product harvested for L4054 and L4055, were sampled and assayed for clonal diversity analysis through sequencing of the CDR3 portion of the T-cell receptors.

Table 76 shows a comparison between Gen 2 and Gen 3 of percentage shared unique CDR3 sequences on L4054 on TIL harvested cell product. 199 sequences are shared between Gen 3 and Gen 2 final product, corresponding to 97.07% of the top 80% of unique CDR3 sequences from Gen 2 shared with Gen 3 final product.

TABLE 76
Comparison of shared uCDR3 sequences between
Gen 2 and Gen 3 processes on L4054.

| # uCDR3 | All uCDR3's | | Top 80% uCDR3's | |
|---|---|---|---|---|
| (% Overlap) | Gen 2 | Gen 3 | Gen 2 | Gen 3 |
| Gen 2-L4054 | 8915 | 4355 (48.85%) | 205 | 199 (97.07%) |
| Gen 3-L4054 | — | 18130 | — | 223 |

Table 77 shows a comparison between Gen 2 and Gen 3 of percentage shared unique CDR3 sequences on L4055 on TIL harvested cell product. 1833 sequences are shared between Gen 3 and Gen 2 final product, corresponding to 99.45% of top 80% of unique CDR3 sequences from Gen 2 shared with Gen 3 final product.

TABLE 77
Comparison of shared uCDR3 sequences between
Gen 2 and Gen 3 processes on L4055.

| # uCDR3 | All uCDR3's | | Top 80% uCDR3's | |
|---|---|---|---|---|
| (% Overlap) | Gen 2 | Gen 3 | Gen 2 | Gen 3 |
| Gen 2-L4055 | 12996 | 6599 (50.77%) | 1843 | 1833 (99.45%) |
| Gen 3-L4055 | — | 27246 | — | 2616 |

CM1 and CM2 media was prepared in advanced without filtration and held at 4 degree C. until use for tumor L4055 to use on Gen 2 and Gen 3 process.

Media was warmed up at 37 degree C. for 24 hours in advance for tumor L4055 on REP initiation day for Gen 2 and Gen 3 process.

LDH was not measured in the supernatants collected on the processes.

M1085T TIL cell count was executed with K2 cellometer cell counter.

On tumor M1085T, samples were not available such as supernatant for metabolic analysis, TIL product for activation and exhaustion markers analysis, telomere length and CD3—TCR vb Analysis.

Conclusions. This example compares 3 independent donor tumors tissue in terms of functional quality attributes, plus extended phenotypic characterization and media consumption among Gen 2 and Gen 3 processes.

Gen 2 and Gen 3 pre-REP and REP expansion comparison were evaluated in terms of total viable cells generated and viability of the total nucleated cell population. TVC cell doses at harvest day was not comparable between Gen 2 (22 days) and Gen 3 (16 days). Gen 3 cell doses were lower than Gen 2 at around 40% of total viable cells collected at harvest.

An extrapolated cell number was calculated for Gen 3 process assuming the pre-REP harvest occurred at day 11 instead day 7 and REP Harvest at Day 22 instead day 16. In both cases, Gen 3 shows a closer number on TVC compared to the Gen 2 process, indicating that the early activation could allow an overall better performance on TIL growth.

In the case of extrapolated value for extra flasks (2 or 3) on Gen 3 process assuming a bigger size of tumor processed, and reaching the maximum number of fragments required per process as described. It was observed that a similar dose can be reachable on TVC at Day 16 Harvest for Gen 3 process compared to Gen 2 process at Day 22. This observation is important and indicates an early activation of the culture can allow better performance of TIL in less processing time.

Gen 2 and Gen 3 pre-REP and REP expansion comparison were evaluated in terms of total viable cells generated and viability of the total nucleated cell population. TVC cell doses at harvest day was not comparable between Gen 2 (22 days) and Gen 3 (16 days). Gen 3 cell doses were lower than Gen 2 at around 40% of total viable cells collected at harvest.

In terms of phenotypic characterization, a higher CD8+ and CD28+ expression was observed on three tumors on Gen 3 process compared to Gen 2 process. This data indicates the Gen 3 process has improved attributes of final TIL product compared to Gen 2.

Gen 3 process showed slightly higher central memory compartments compared to Gen 2 process.

Gen 2 and Gen 3 process showed comparable activation and exhaustion markers, despite the shorter duration of the Gen 3 process.

IFN gamma (IFNγ) production was 3 times higher on Gen 3 final product compared to Gen 2 in the three tumors analyzed. This data indicates the Gen 3 process generated a highly functional and more potent TIL product as compared to the Gen 2 process, possibly due to the higher expression of CD8 and CD28 expression on Gen 3. Phenotypic characterization suggested positive trends in Gen 3 toward CD8+, CD28+ expression on three tumors compared to Gen 2 process.

Telomere length on TIL final product between Gen 2 and Gen 3 were comparable.

Glucose and Lactate levels were comparable between Gen 2 and Gen 3 final product, suggesting the levels of nutrients on the media of Gen 3 process were not affected due to the non-execution of volume reduction removal in each day of the process and less volume media overall in the process, compared to Gen 2.

Overall Gen 3 process showed a reduction almost two times of the processing time compared to Gen 2 process, which would yield a substantial reduction on the cost of goods (COGs) for TIL product expanded by the Gen 3 process.

IL-2 consumption indicates a general trend of IL-2 consumption on Gen 2 process, and in Gen 3 process IL-2 was higher due to the non-removal of the old media.

The Gen 3 process showed a higher clonal diversity measured by CDR3 TCRab sequence analysis.

The addition of feeders and OKT-3 on day 0 of the pre-REP allowed an early activation of TIL and overall a better growth TIL performance using the Gen 3 process.

Table 78 describes various embodiments and outcomes for the Gen 3 process as compared to the current Gen 2 process.

TABLE 78
Exemplary Gen 2 and Gen 3 process features.

| Step | Process Gen 2 embodiment | Process Gen 3 embodiment |
|---|---|---|
| Pre REP- | ≤50 fragments | ≤240 fragments |
| day 0 | 1X G-Rex 100MCS | ≤60 fragments/flask |
| | 1 L media | ≤4 flasks |
| | IL-2 (6000 IU/mL) | ≤2 L media (500 mL/flask) |
| | 11 days | IL-2 (6000 IU/mL) |
| | | 2.5 × 10⁸ feeder cells/flask |
| | | 15 ug OKT3/flask |
| REP | Fresh TIL direct to REP | Fresh TIL direct to REP |
| Initiation | Day 11 | Day 7 |
| | ≤200e⁶ viable cells | Activate entire culture |
| | 5 × 10⁹ feeder cells | 5 × 10⁸ feeder cells |
| | G-Rex 500MCS | 30 μg OKT3/flask |
| | 5 L CM2 media + IL-2 (3000 | G-Rex 100MCS |
| | IU/mL) | 500 mL media + IL-2 (6000 |
| | 150 μg OKT3 | IU/mL) |
| TIL Sub- | ≤5 G-Rex 500MCS | ≤4 G-Rex 500MCS |
| culture or | ≤1 × 10 viable cells/flask | Scale up entire culture |
| Scale up | 5 L/flask | 4 L/flask |
| | Day 16 | Day 10-11 |
| Harvest | Harvest Day 22, | Harvest Day 16 |
| | LOVO automated cell washer | LOVO automated cell washer |
| | 2 wash cycles | 5 wash cycles |
| Final | Cryopreserved Product | Cryopreserved product |
| formulation | 300 IU/mL IL2- CS10 in LN₂, | 300 IU/mL IL-2-CS10 in LN₂, |
| | multiple aliquots | multiple aliquots |
| Process | 22 days | 16 days |
| time | | |

### Example 7: An Exemplary Embodiment of the Gen 3 Expansion Process at Day 0

Prepared tumor wash media. Media warmed prior to start. Added 5 mL of gentamicin (50 mg/mL) to the 500 mL bottle of HBSS. Added 5 mL of Tumor Wash Media to a 15 mL conical to be used for OKT3 dilution. Store at room temperature (RT).

Prepared feeder cell bags. Sterilely transferred feeder cells to feeder cell bags and stored at 37° C. until use or freeze. Counted feeder cells if at 37° C. Thawed and then counted feeder cells if frozen.

Optimal range for the feeder cell concentration is between 5×10⁴ and 5×10⁶ cells/mL. Prepared four conical tubes with 4.5 mL of AIM-V. Added 0.5 mL of cell fraction for each cell count.

If total viable feeder cell number was ≥1×10⁹ cells, proceeded to the next step to adjust the feeder cell concentration. Calculated the volume of feeder cells to remove from the first feeder cell bag in order to add 1×10⁹ cells to a second feeder cell bag.

Using the p1000 micropipette, transferred 900 μL of Tumor Wash Media to the OKT3 aliquot (100 μL). Using a syringe and sterile technique, drew up 0.6 mL of OKT3 and added into the second feeder cell bag. Adjusted media volume to a total volume of 2 L. Transferred the second feeder cells bag to the incubator.

OKT3 formulation details: OKT3 may be aliquoted and frozen in original stock concentration from the vial (1 mg/mL) in 100 μL aliquots. ˜10× aliquots per 1 mL vial. Stored at −80 C. Day 0: 15 μg/flask, i.e. 30 ng/mL in 500 mL-60 μL max˜1 aliquot.

Prepared tumor samples. Obtained 6-well plate and 100 mm petri dishes (4 total). Labeled the 6 well plate ‘Excess Tumor Pieces’. Labeled one each of the four 100 mm petri dishes as ‘Wash_01’, ‘Wash_02’, ‘Wash_03’, ‘Wash_04’, and ‘Holding’.

Added 5 mL of Tumor Wash Medium into all wells of the 6-well plate labelled Excess Tumor Pieces. Kept the Tumor Wash Medium available for further use in keeping the tumor hydrated during dissection.

Added 50 mL of Tumor Wash Medium to each 100 mm petri dish labelled Wash_01, Wash_02, Wash_03, and Holding. Using a marker, label each petri dish as Dissection 1 through Dissection 4. Incubated the tumor at ambient temperature in Wash_01 for ≥3 min. Incubated the tumor at ambient temperature in Wash_02 for ≥3 min. Incubated the tumor at ambient temperature in Wash_03 for ≥3 min. After washes were completed, moved tumor to the ‘Holding’ dish to ensure tissue stays hydrated.

While tumor incubations were in progress, transferred 10 mL of tumor shipping medium into a tube labelled Tumor Shipping Medium. Drew 10 mL of the Tumor Shipping Medium into a syringe and inoculated one each anaerobic and aerobic sterility bottle with 5 mL of tumor shipping medium.

Placed ruler under the petri dish lid for the entirety of the dissection process. Measured and recorded length of the tumor and the number of fragments. Dissected the tumor into four intermediate pieces or group into four groups of equivalent volume and conserving the tumor structure of each intermediate piece. Keep tumor pieces hydrated.

Transferred any intermediate tumor pieces not being actively dissected to the Holding dish to keep the tissue hydrated.

Dissected the tumor into 27 mm³ fragments (3×3×3 mm), using the ruler under the Dissection dish lid as a reference. Dissected intermediate fragment until 60 fragments were reached. Counted total number of final fragments and prepared G-Rex 100MCS flasks according to the number of final fragments generated (generally 60 fragments per flask).

Retained favorable tissue fragments in the conical tubes labeled as Fragments Tube 1 through Fragments Tube 4. Calculated the number of G-Rex 100MCS flasks to seed with feeder cell suspension according to the number of fragments tubes originated.

Removed feeder cells bag from the incubator and seed the G-Rex 100MCS. Label as D0 (Day 0).

Tumor fragment addition to culture in G-Rex 100 MCS. Under sterile conditions, unscrewed the cap of the G-Rex 100MCS labelled Tumor Fragments Culture (D0) 1 and the 50 mL conical tube labelled Fragments Tube. Swirled the opened Fragments Tube 1 and, at the same time, slightly lifted the cap of the G-Rex100MCS. Added the medium with the fragments to the G-Rex100MCS while being swirled. Recorded the number of fragments transferred into the G-Rex100MCS.

Once the fragments were located at the bottom of the GREX flask, drew 7 mL of media and created seven 1 mL aliquots-5 mL for extended characterization and 2 mL for sterility samples. Stored the 5 aliquots (final fragment culture supernatant) for extended characterization at −20° C. until needed.

Inoculated one anaerobic BacT/Alert bottle and one aerobic BacT/Alert bottle each with 1 mL of final fragment culture supernatant. Repeat for each flask sampled.

### Example 8: An Exemplary Embodiment of the Gen 3 Expansion Process at Day 7-8

Prepared feeder cell bags. Thawed feeder bags for 3-5 minutes in 37° C. water bath when frozen. Counted feeder cells if frozen.

Optimal range for the feeder cell concentration is between 5×10⁴ and 5×10⁶ cells/mL. Prepared four conical tubes with 4.5 mL of AIM-V. Added 0.5 mL of cell fraction for each cell count into a new cryovial tube. Mixed the samples well and proceeded with the cell count.

If total viable feeder cell number was ≥2×10⁹ cells, proceeded to the next step to adjust the feeder cell concentration. Calculated the volume of feeder cells to remove from the first feeder cell bag in order to add 2×10⁹ cells to the second feeder cell bag.

Using the p1000 micropipette, transfer 900 μL of HBSS to a 100 μL OKT3 aliquot. Mix by pipetting up and down 3 times. Prepared two aliquots.

OKT3 formulation details: OKT3 may be aliquoted and frozen in original stock concentration from the vial (1 mg/mL) in 100 μL aliquots. ˜10× aliquots per 1 mL vial. Stored at −80 C. Day 7/8: 30 μg/flask, i.e. 60 ng/mL in 500 mL-120 μl max˜2 aliquots.

Using a syringe and sterile technique, drew up 0.6 mL of OKT3 and added into the feeder cell bag, ensuring all added. Adjusted media volume to a total volume of 2 L. Repeated with second OKT3 aliquot and added to the feeder cell bag. Transferred the second feeder cells bag to the incubator.

Preparation of G-Rex100MCS flask with feeder cell suspension. Recorded the number of G-Rex 100MCS flasks to process according to the number of G-Rex flasks generated on Day 0. Removed G-Rex flask from incubator and removed second feeder cells bag from incubator.

Removal of supernatant prior to feeder cell suspension addition. Connected one 10 mL syringe to the G-Rex100 flask and drew up 5 mL of media. Created five 1 mL aliquots −5 mL for extended characterization and stored the 5 aliquots (final fragment culture supernatant) for extended characterization at −20° C. until requested by sponsor. Labeled and repeated for each G-Rex100 flask.

Prepare 5-20×1 mL samples for characterization, depending on number of flasks:

$$5 mL = 1 flask$$

$$10 mL = 2 flasks$$

$$15 mL = 3 flasks$$

$$20 mL = 4 flasks$$

Continued seeding feeder cells into the G-Rex100 MCS and repeated for each G-Rex100 MCS flask. Using sterile transfer methods, gravity transferred 500 mL of the second feeder cells bag by weight (assume 1 g=1 mL) into each G-Rex 100MCS flask and recorded amount. Labeled as Day 7 culture and repeated for each G-Rex100 flask. Transferred G-Rex 100MCS flasks to the incubator.

### Example 9: An Exemplary Embodiment of the Gen 3 Expansion Process at Day 10-11

Removed the first G-Rex 100MCS flask and using sterile conditions removed 7 mL of pre-process culture supernatant using a 10 mL syringe. Created seven 1 mL aliquots-5 mL for extended characterization and 2 mL for sterility samples.

Mixed the flask carefully and using anew 10 mL syringe remove 10 mL supernatant and transfer to a 15 mL tube labelled as D10/11 mycoplasma supernatant.

Mixed the flask carefully and using a new syringe removed the volume below according to how many flasks were to be processed:

$$1 flask = 40 mL$$

$$2 flask = 20 mL / flask$$

$$3 flask = 13.3 mL / flask$$

$$4 flask = 10 mL / flask$$

A total of 40 mL should be pulled from all flasks and pooled in a 50 mL conical tube labeled ‘Day 10/11 QC Sample’ and stored in the incubator until needed. Performed a cell count and allocated the cells.

Stored the 5 aliquots (pre-process culture supernatant) for extended characterization at ≤−20° C. until needed. Inoculated one anaerobic BacT/Alert bottle and one aerobic BacT/Alert bottle each with 1 mL of pre-process culture supernatant.

Continued with cell suspension transferred to the G-Rex 500MCS and repeated for each G-Rex 100MCS. Using sterile conditions, transferred the contents of each G-Rex 100MCS into a G-Rex 500MCS, monitoring about 100 mL of fluid transfer at a time. Stopped transfer when the volume of the G-Rex 100MCS was reduced to 500 mL.

During transfer step, used 10 mL syringe and drew 10 mL of cell suspension into the syringe from the G-Rex 100MCS. Followed the instructions according to the number of flasks in culture. If only 1 flask: Removed 20 mL total using two syringes. If 2 flasks: removed 10 mL per flask. If 3 flasks: removed 7 mL per flask. If 4 flasks: removed 5 mL per flask. Transferred the cell suspension to one common 50 mL conical tube. Keep in the incubator until the cell count step and QC sample. Total number of cells needed for QC was 20e6 cells: 4×0.5 mL cell counts (cell counts were undiluted first).

The quantities of cells needed for assays are as follows:
10×10⁶ cells minimum for potency assays, such as those described herein, or for an IFN-γ or granzyme B assay
1×10⁶ cells for mycoplasma
5×10⁶ cells for flow cytometry for CD3+/CD45+

Transferred the G-Rex 500MCS flasks to the incubator.

Prepared QC Samples. At least 15×10⁸ cells were needed for the assays in this embodiment. Assays included: Cell count and viability; Mycoplasma (1×10⁶ cells/average viable concentration;) flow (5×10⁶ cells/average viable concentration;) and IFN-g assay (5×10⁶ cells-1×10⁶ cells; 8-10×10⁶ cells are required for the IFN-γ assay.

Calculated the volume of cells fraction for cryopreservation at 10×10⁶ cells/mL and calculated the number of vials to prepare.

### Example 10: An Exemplary Embodiment of the Gen 3 Expansion Process at Day 16-17

Wash Buffer preparation (1% HSA Plasmalyte A). Transfer HSA and Plasmalyte to 5 L bag to make LOVO wash buffer. Using sterile conditions, transferred a total volume of 125 mL of 25% HSA to the 5 L bag. Stored at room temperature.

Removed and transferred 10 mL or 40 mL of wash buffer in the ‘IL-2 6×10⁴ IU/mL’ tube (10 mL if IL-2 was prepared in advance or 40 mL if IL-2 was prepared fresh).

Calculated volume of reconstituted IL-2 to add to Plasmalyte+1% HSA: volume of reconstituted IL-2=(Final concentration of IL-2×Final volume)/specific activity of the IL-2 (based on standard assay). The Final Concentration of IL-2 was 6×10⁴ IU/mL. The final volume was 40 mL.

Removed calculated initial volume of IL-2 needed of reconstituted IL-2 and transfer to the ‘IL-2 6×10⁴ IU/mL’ tube. Added 100 μp of IL-2 6×10⁶ IU/mL from the aliquot prepared in advance to the tube labelled ‘IL-2 6×10⁴ IU/mL’ containing 10 mL of LOVO wash buffer.

Removed about 4500 mL of supernatant from the G-Rex 500MCS flasks. Swirled the remaining supernatant and transferred cells to the Cell Collection Pool bag. Repeated with all G-Rex 500MCS flasks.

Removed 60 mL of supernatant and add to supernatant tubes for quality control assays, including mycoplasma detection. Stored at +2-8° C.

Cell collection. Counted cells. Prepare four 15 mL conical vials with 4.5 mL of AIM-V. These may be prepared in advance. Optimal range=is between 5×10⁴ and 5×10⁶ cells/mL. (1:10 dilution was recommended). For 1:10 dilution, to 4500 μL of AIM V prepared previously, add 500 μL of CF. Recorded dilution factor.

$$Calculated the TC ( Total Cells ) pre - LOVO ( live + dead ) = Average Total Cell Concentration ( TC conc pre LOVO ) ( live + dead ) \times Volume of Source bag$$

$$Calculated the TVC ( Total Viable Cells ) pre - LOVO ( live ) = Average Total Viable Cell Concentration ( TVC p re LOVO ) ( live ) \times Volume of LOVO Source bag$$

When the total cell (TC) number was >5×10⁹, remove 5×10⁸ cells to be cryopreserved as MDA retention samples. 5×10⁸÷avg TC concentration (step 14.44)=volume to remove.

When the total cell (TC) number was ≤5×10⁹, remove 4×10⁶ cells to be cryopreserved as MDA retention samples. 4×10⁶÷avg TC concentration=volume to remove.

Used an appropriately sized syringe to remove the required volume from the LOVO Source Bag. Retained in incubator until cryopreservation steps.

When the total cell number was determined, the number of cells to remove should allow retention of 150×10⁹ viable cells. Confirm TVC pre-LOVO 5×10⁸ or 4×10⁶ or not applicable. Calculated the volume of cells to remove.

Calculated the remaining Total Cells Remaining in Bag. Calculated the TC (Total Cells) pre-LOVO. [Avg. Total cell concentration×Remaining Volume=TC pre-LOVO Remaining]

According to the total number of cells remaining, the corresponding process in Table 79 is selected.

TABLE 79
Total number of cells.

| Total cells: | Retentate (mL) |
|---|---|
| 0 < Total cells ≤ 31 × 10⁹ | 115 |
| 31 × 10⁹ < Total cells ≤ 71 × 10⁹ | 165 |
| 71 × 10⁹ < Total Cells ≤ 110 × 10⁹ | 215 |
| 110 × 10⁹ < Total Cells ≤ 115 × 10⁹ | 265 |

Chose the volume of IL-2 to add corresponding to the used process. Volume calculated as: Retentate Volume×2×300 IU/mL=IU of IL-2 required. IU of IL-2 required/6×10⁴IU/mL=Volume of IL-2 to add Post LOVO bag. Recorded all volumes added. Obtained samples in cryovial for further analyses.

Mixed the cell product well. Sealed all bags for further processing, included cryopreservation when applicable.

Performed endotoxin, IFN-γ, sterility, and other assays as needed on cryovial samples obtained.

### Example 11: An Exemplary Gen 3 Process (Also Referred to as Gen 3.1)

This example describes further studies regarding the “Comparability between the Gen 2 and Gen 3 processes for TIL expansion”. The Gen 3 process was modified to include an activation step early in the process with the goal of increasing the final total viable cell (TVC) output to be comparable (or better) to that in Gen 2, while maintaining the phenotypic and functional profiles as previously seen.

The scope of this example involves assessment of TVC output through introduction of an activation step to the cultured tumor fragments on Day 0; demonstrating comparability in terms of functional and extended phenotypic characterization with the Gen 3 standard, as well as a control arm, across two independent patient tumors; and analysis of media consumption and metabolite production to confirm processing parameters were maintained at physiologic conditions.

All runs for this example were performed at full-scale platform using commercial donor tumor tissue as the starting material.

A Gen 3 embodiment was modified as a further embodiment and is referred to herein in this example as Gen 3.1.

In an embodiment, the Gen 3.1 TIL manufacturing process has four operator interventions:
1) Tumor Fragment Isolation and Activation: On Day 0 of the process the tumor was dissected and the final fragments generated awe˜3×3 mm each (up to 240 fragments total) and cultured in 1-4 G-Rex100MCS flasks. Each flask contained up to 60 fragments, 500 mL of CM1 or DM1 media, and supplemented with 6,000 IU rhIL-2, 15 μg OKT3, and 2.5×10⁸ irradiated allogeneic mononuclear cells. The culture was incubated at 37° C. for 6-8 days.
2) TIL Culture Reactivation: On Day 7-8 the culture was supplemented through slow addition of CM2 or DM1 media supplemented with 6,000 IU rhIL-2, 30 μg OKT3, and 5×10⁸ irradiated allogeneic mononuclear cells in both cases. Care was taken to not disturb the existing cells at the bottom of the flask. The culture was incubated at 37° C. for 3-4 days.
3) Culture Scale Up: Occurs on day 10-11. During the culture scale-up, the entire contents of the G-Rex100MCS was transferred to a G-Rex500MCS flask containing 4 L of CM4 or DM2 supplemented with 3,000 IU/mL of IL-2 in both cases. Flasks were incubated at 37° C. for 5-6 days until harvest.
4) Harvest/Wash/Formulate: On day 16-17 the flasks are volume reduced and pooled. Cells were concentrated and washed with PlasmaLyte A pH 7.4 containing 1% HSA. The washed cell suspension was formulated at a 1:1 ratio with CryoStor10 and supplemented with rhIL-2 to a final concentration of 300 IU/mL.

The DP was cryopreserved with a controlled rate freeze and stored in vapor phase liquid nitrogen. *Complete Standard TIL media 1, 2, or 4 (CM1, CM2, CM4) could be substituted for CTS™OpTmizer™ T-Cell serum free expansion Medium, referred to as Defined Medium (DM1 or DM2), as noted above.

Process description. On day 0, the tumor was washed 3 times, then fragmented in 3×3×3 final fragments. Once the whole tumor was fragmented, then the final fragments were randomized equally and divided into three pools. One randomized fragment pool was introduced to each arm, adding the same number of fragments per the three experimental matrices.

Tumor L4063 expansion was performed with Standard Media and tumor L4064 expansion was performed with Defined Media (CTS OpTmizer) for the entire TIL expansion process. Components of the media are described herein.

CM1 Complete Media 1: RPMI+Glutamine supplemented with 2 mM Glutamax, 10% Human AB Serum, Gentamicin (50 ug/mL), 2-Mercaptoethanol (55 uM). Final media formulation supplemented with 6000 IU/mL IL-2.

CM2 Complete Media 2: 50% CM1 medium+50% AIM-V medium. Final media formulation supplemented with 6000 IU/mL IL-2.

CM4 Complete Media 4: AIM-V supplemented with Glutamax (2 mM). Final media formulation supplemented with 3000 IU/mL IL-2.

CTS OpTmizer CTS™OpTmizer™ T-Cell Expansion Basal Medium supplemented with CTS™ OpTmizer™ T-Cell Expansion Supplement (26 mL/L).

DM1: CTS™OpTmizer™ T-Cell Expansion Basal Medium supplemented with CTS™ OpTmizer™ T-Cell Expansion Supplement (26 mL/L), and CTS™ Immune Cell SR (3%), with Glutamax (2 mM). Final formulation supplemented with 6,000 IU/mL of IL-2.

DM2: CTS™OpTmizer™ T-Cell Expansion Basal Medium supplemented with CTS™ OpTmizer™ T-Cell Expansion Supplement (26 mL/L), and CTS™ Immune Cell SR (3%), with Glutamax (2 mM). Final formulation supplemented with 3,000 IU/mL of IL-2.

All types of media used, i.e., Complete (CM) and Defined (DM) media, were prepared in advance, held at 4° C. degree until the day before use, and warmed at 37° C. in an incubator for up to 24 hours in advance prior to process day.

TIL culture reactivation occurred on Day 7 for both tumors. Scale-up occurred on day 10 for L4063 and day 11 for L4064. Both cultures were harvested and cryopreserved on Day 16.

Results Achieved. Cells counted and % viability for Gen 3.0 and Gen 3.1 processes were determined. Expansion in all the conditions followed details described in this example.

For each tumor, the fragments were divided into three pools of equal numbers. Due to the small size of the tumors, the maximum number of fragments per flask was not achieved. For the three different processes, the total viable cells and cell viability were assessed for each condition. Cell counts were determined as TVC on day 7 for reactivation, TVC on day 10 (L4064) or day 11 (L4063) for scale-up, and TVC at harvest on day 16/17.

Cell counts for Day 7 and Day 10/11 were taken FIO. Fold expansion was calculated by dividing the harvest day 16/17 TVC by the day 7 reactivation day TVC. To compare the three arms, the TVC on harvest day was divided by the number of fragments added in the culture on Day 0 in order to calculate an average of viable cells per fragment.

Cell counts and viability assays were performed for L4063 and L4064. The Gen 3.1-Test process yielded more cells per fragment than the Gen 3.0 Process on both tumors.

Total viable cell count and fold expansion: % Viability during the process. On reactivation, scale up and harvest the percent viability was performed on all conditions. On day 16/17 harvest, the final TVC were compared against release criteria for % viability. All of the conditions assessed surpassed the 70% viability criterion and were comparable across processes and tumors.

Immunophenotyping—Phenotypic characterization on TIL final product. The final products were subjected to flow cytometry analysis to test purity, differentiation, and memory markers. Percent populations were consistent for TCRα/β, CD4+ and CD8+ cells for all conditions.

Extended phenotypic analysis of REP TIL was performed. TIL product showed a higher percentage of CD4+ cells for Gen 3.1 conditions compared to Gen 3.0 on both tumors, and higher percentage of CD28+ cells from CD8+ population for Gen 3.0 compared to Gen 3.1 conditions on both conditions.

TIL harvested from the Gen 3.0 and Gen 3.1 processes showed comparable phenotypic markers as CD27 and CD56 expression on CD4+ and CD8+ cells, and a comparable CD28 expression on CD4+ gated cells population. Memory markers comparison on TIL final product:

Frozen samples of TIL harvested on day 16 were stained for analysis. TIL memory status was comparable between Gen 3.0 and Gen 3.1 processes. Activation and exhaustion markers comparison on TIL final product:

Activation and exhaustion markers were comparable between the Gen 3.0 and Gen 3.1 processes gated on CD4+ and CD8+ cells.

Interferon gamma secretion upon restimulation. Harvested TIL underwent an overnight restimulation with coated anti-CD3 plates for L4063 and L4064. Higher production of IFNγ from the Gen 3.1 process was observed in the two tumors analyzed compared to Gen 3.0 process.

Measurement of IL-2 levels in culture media. To compare the levels of IL-2 consumption between all of the conditions and processes, cell supernatants were collected at initiation of reactivation on Day 7, at scale-up Day 10 (L4064)/11 (L4063), and at harvest Day 16/17, and frozen. The supernatants were subsequently thawed and then analyzed. The quantity of IL-2 in cell culture supernatant was measured by the manufacturer protocol.

Overall Gen 3 and Gen 3.1 processes were comparable in terms of IL-2 consumption during the complete process assessed across same media conditions. IL-2 concentration (pg/mL) analysis on spent media collected for L4063 and L4064.

Metabolite analysis. Spent media supernatants was collected from L4063 and L4064 at reactivation initiation on day 7, scale-up on day 10 (L4064) or day 11 (L4063), and at harvest on days 16/17 for L4063 and L4064, for every condition. Supernatants were analyzed on a CEDEX Bio-analyzer for concentrations of glucose, lactate, glutamine, GlutaMax, and ammonia.

Defined media has a higher glucose concentration of 4.5 g/L compared to complete media (2 g/L). Overall, the concentration and consumption of glucose were comparable for Gen 3.0 and Gen 3.1 processes within each media type.

An increase in lactate was observed for both tumors, L4063 and L4064, for all test conditions. The increase in lactate was comparable between the Gen 3.0 and Gen 3.1 conditions and between the two media used for reactivation expansion (complete media for L4063 and defined media for L4064).

In the case of L4063, the standard basal media contained 2 mM L-glutamine and was supplemented with 2 mM GlutaMax to compensate for the natural degradation of L-glutamine in culture conditions to L-glutamate and ammonia.

For L4064 tumor, defined (serum free) media used did not contain L-glutamine on the basal media, and was supplemented only with GlutaMax to a final concentration of 2 mM. GlutaMax is a dipeptide of L-alanine and L-glutamine, is more stable than L-glutamine in aqueous solutions and does not spontaneously degrade into glutamate and ammonia. Instead, the dipeptide is gradually dissociated into the individual amino acids, thereby maintaining a lower but sufficient concentration of L-glutamine to sustain robust cell growth.

For L4063, the concentration of glutamine and GlutaMax slightly decreased on the scale-up day, but at harvest day showed an increase to similar or closer levels compared to reactivation day. For L4064, glutamine and GlutaMax concentration showed a slight degradation in a similar rate between different conditions, during the whole process.

As expected, ammonia concentrations were higher for L4063 (grown in standard media containing 2 mM glutamine+2 mM GlutaMax) than L4064 (grown in defined media containing 2 mM GlutaMax). Further, as expected, there was a gradual increase or accumulation of ammonia over the course of the culture. There were no differences in ammonia concentrations across the three different test conditions.

Telomere repeats by Flow—FISH. Flow-FISH technology was used to measure the average length of the telomere repeat on L4063 and L4064 under Gen 3 and Gen 3.1 processes. The determination of a relative telomere length (RTL) was calculated using Telomere PNA kit/FITC for flow cytometry analysis from DAKO. Telomere assay was performed. Telomere length in samples of L4063 and L4064 were compared to a control cell line (1301 leukemia). The control cell line is a tetraploid cell line having long stable telomeres that allows calculation of a relative telomere length. Gen 3 and Gen 3.1 processes assessed in both tumors showed comparable telomere length. TCR Vβ repertoire Analysis

To determine the clonal diversity of the cell products generated in each process, TIL final products were assayed for clonal diversity analysis through sequencing of the CDR3 portion of the T-cell receptors.

Three parameters were compared between the three conditions:
Diversity index of Unique CDR3 (uCDR3)
% shared uCDR3
For the top 80% of uCDR3:
   Compare the % shared uCDR3 copies
   Compare the frequency of unique clonotypes

Control and Gen 3.1 Test, percentage shared unique CDR3 sequences on L4063 on TIL harvested cell product for: 975 sequences are shared between Gen 3 and Gen 3.1 Test final product, equivalent to 88% of top 80% of unique CDR3 sequences from Gen 3 shared with Gen 3.1 Test final product.

Control and Gen 3.1 Test, percentage shared unique CDR3 sequences on L4064 on TIL harvested cell product for: 2163 sequences are shared between Gen 3 and Gen 3.1 Test final product, equivalent to 87% of top 80% of unique CDR3 sequences from Gen 3 shared with Gen 3.1 Test final product.

The number of unique CD3 sequences identified from 1×10⁶ cells collected on Harvest day 16, for the different processes. Gen 3.1 Test condition showed a slightly higher clonal diversity compared to Gen 3.0 based on the number of unique peptide CDRs within the sample.

The Shannon entropy diversity index is a more reliable and common metric for comparison, because Gen 3.1 conditions on both tumors showed slightly higher diversity than Gen 3 process, suggesting that TCR VP repertoire for Gen 3.1 Test condition is more polyclonal than the Gen 3.0 process.

Additionally, the TCR Vβ repertoire for Gen 3.1 Test condition showed more than 87% overlap with the corresponding repertoire for Gen 3.0 process on both tumor L4063 and L4064.

The value of IL-2 concentration on spent media for Gen 3.1 Test L4064 on reactivation day was below to the expected value (similar to Gen 3.1 control and Gen 3.0 condition).

The low value could be due to a pipetting error, but because of the minimal sample taken it was not possible to repeat the assay.

Spent media from scale up day 10/11 on sample L4064 was not collected, and not included in the analysis of IL-2 concentration and metabolite analysis on supernatant.

Conclusions. Gen 3.1 test condition including feeders and OKT-3 on Day 0 showed a higher TVC of cell doses at Harvest day 16 compared to Gen 3.0 and Gen 3.1 control. TVC on the final product for Gen 3.1 test condition was around 2.5 times higher than Gen 3.0.

Gen 3.1 test condition with the addition of OKT-3 and feeders on day 0, for both tumors L4063 and L4064, reached a maximum capacity of the flask at harvest. Under these conditions, if a maximum of 4 flasks on day 0 is initiated, the final cell dose could be between 80-100×10⁹ TILs.

All the quality attributes such as phenotypic characterization including purity, exhaustion, activation and memory markers on final TIL product were maintained and comparable between Gen 3.1 Test and Gen 3.0 process. Telomere length on TIL final product and IL-2 consumption on spent media were comparable between Gen 3.0 and Gen 3.1 processes.

IFN-γ production on final TIL product was 3 times higher on Gen 3.1 with feeder and OKT-3 addition on day 0, compared to Gen 3.0 in the two tumors analyzed, suggesting Gen 3.1 process generated a potent TIL product.

No differences observed in glucose or lactate levels across test conditions. No differences observed on glutamine and ammonia between Gen 3.0 and Gen 3.1 processes across media conditions. The low levels of glutamine on the media are not limiting cell growth and suggest the addition of GlutaMax only in media is sufficient to give the nutrients needed to make cells proliferate.

The scale up day for L4063 and L4064 was on day 11 and day 10 respectively and did not show major differences in terms of cell number reached on the harvest day of the process and metabolite consumption was comparable in both cases during the whole process. This observation suggests of Gen 3.0 optimized process can have flexibility on processing days, thereby facilitating flexibility in the manufacturing schedule.

Gen 3.1 process with feeder and OKT-3 addition on day 0 showed a higher clonal diversity measured by CDR3 TCRab sequence analysis compared to Gen 3.0.

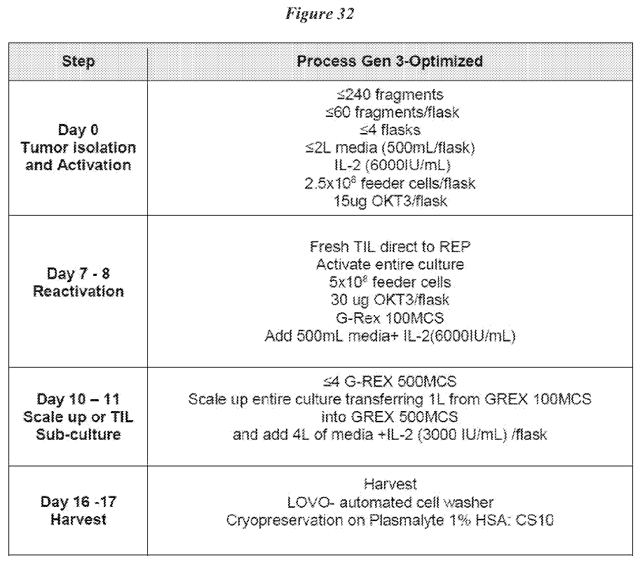

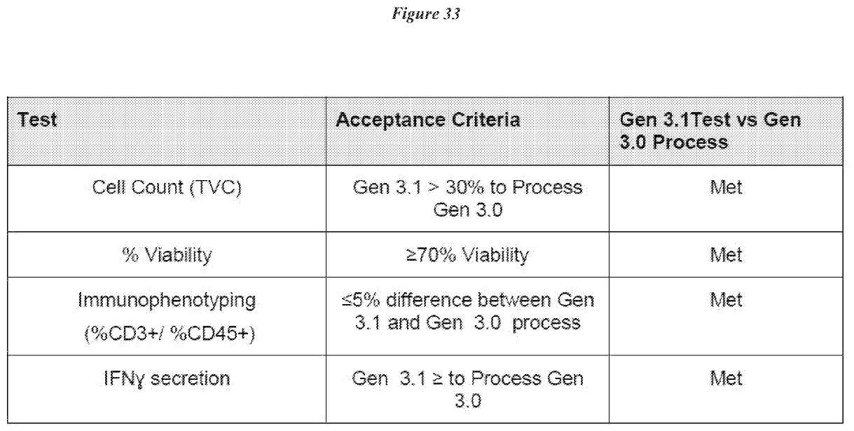

FIG. 32 describes an embodiment of the Gen 3 process (Gen 3 Optimized process). Standard media and CTS Optimizer serum free media can be used for Gen 3 Optimized process TIL expansion. In case of CTS Optimizer serum free media is recommended to increase the GlutaMax on the media to final concentration 4 mM.

Feasibility was established for all study conditions in all experiments. Across all the experiments and conditions and between the donor tumor tissue, all the experiments were performed utilizing the same lots of critical raw material such as IL-2, Human Serum-AB, allogeneic feeder cells, OKT-3.

Comparability was determined by the ability of any arm of the study to meet release criteria of the clinical product according to prior specifications for cryopreserved day 22 TIL products.

### Example 12: Tumor Expansion Processes with Defined Medium

The processes disclosed above, including the Gen 2 and Gen 3 processes, may be performed substituting the CM1 and CM2 media with a defined medium (e.g., CTS™ OpTmizer™ T-Cell Expansion SFM, ThermoFisher, including for example DM1 and DM2).

### Example 13: Exemplary Production of a Cryopreserved TIL Cell Therapy

This example describes an exemplary cGMP manufacture of TIL cell therapies in G-Rex flasks (or alternatively in gas-permeable bags) according to current Good Manufacturing Practices.

TABLE 80
Process Expansion Exemplary Plan.

| Estimated Day | | | | Estimated Total |
|---|---|---|---|---|
| (post-seed) | Activity | Target Criteria | Anticipated Vessels | Volume (mL) |
| 0 | Tumor | ≤50 desirable tumor fragments | G-Rex 100MCS 1 flask | ≤1000 |
| | Dissection | per G-Rex 100MCS | | |
| 11 | REP Seed | 5-200 × 10⁶viable cells | G-Rex 500MCS 1 flask | ≤5000 |
| | | per G-Rex 500MCS | | |
| 16 | REP Split | 1 × 10⁹viable cells | G-Rex 500MCS ≤5 | ≤25000 |
| | | per G-Rex 500MCS | flasks | |
| 22 | Harvest | Total available cells | 3-4 CS-750 bags | ≤530 |

TABLE 81
Flask Volumes.

| | Working |
|---|---|
| Flask Type | Volume/Flask (mL) |
| G-Rex 100MCS | 1000 |
| G-Rex 500MCS | 5000 |

Day 0 CM1 Media Preparation. In the BSC added reagents to RPMI 1640 Media bottle. Added the following reagents: Heat Inactivated Human AB Serum (100.0 mL); GlutaMax (10.0 mL); gentamicin sulfate, 50 mg/mL (1.0 mL); 2-mercaptoethanol (1.0 mL)

Removed unnecessary materials from BSC. Passed out media reagents from BSC, left gentamicin sulfate and HBSS in BSC for formulated wash media preparation.

Thawed IL-2 aliquot. Thawed one 1.1 mL IL-2 aliquot (6×106 IU/mL) (BR71424) until all ice had melted. Recorded IL-2 lot #and expiry

Transferred IL-2 stock solution to media. In the BSC, transferred 1.0 mL of IL-2 stock solution to the CM1 Day 0 media bottle prepared. Added CM1 day 0 media 1 bottle and IL-2 (6×106 IU/mL) 1.0 mL.

Passed G-Rex100MCS into BSC. Aseptically passed G-Rex100MCS (W3013130) into the BSC.

Pumped all complete CM1 day 0 media into G-Rex100MCS flask (tissue fragments conical or GRex100MCS).

Day 0 Tumor Wash Media Preparation. In the BSC, added 5.0 mL Gentamicin (W3009832 or W3012735) to 1×500 mL HBSS Media (W3013128) bottle. Added per bottle: HBSS (500.0 mL); gentamicin sulfate, 50 mg/mL (5.0 mL). Filtered HBSS containing gentamicin prepared through a 1 L 0.22-micron filter unit (W1218810).

Day 0 Tumor Processing. Obtained tumor specimen and transferred into suite at 2-8° C. immediately for processing.

Aliquoted tumor wash media. Tumor wash 1 is performed using 8″ forceps (W3009771). The tumor is removed from the specimen bottle and transferred to the “Wash 1” dish prepared. This is followed by tumor wash 2 and tumor wash 3.

Measured and assessed tumor. Assessed whether >30% of entire tumor area observed to be necrotic and/or fatty tissue. Clean up dissection if applicable. If tumor was large and >30% of tissue exterior was observed to be necrotic/fatty, performed “clean up dissection” by removing necrotic/fatty tissue while preserving tumor inner structure using a combination of scalpel and/or forceps.

Dissect tumor. Using a combination of scalpel and/or forceps, cut the tumor specimen into even, appropriately sized fragments (up to 6 intermediate fragments). Transferred intermediate tumor fragments. Dissected tumor fragments into pieces approximately 3×3×3 mm in size. Stored Intermediate Fragments to prevent drying.

Repeated intermediate fragment dissection. Determined number of pieces collected. If desirable tissue remains, selected additional favorable tumor pieces from the “favorable intermediate fragments” 6-well plate to fill the drops for a maximum of 50 pieces.

Prepared conical tube. Transferred tumor pieces to 50 mL conical tube. Prepared BSC for G-Rex100MCS. Removed G-Rex100MCS from incubator. Aseptically passed G-Rex100MCS flask into the BSC. Added tumor fragments to G-Rex100MCS flask. Evenly distributed pieces.

Incubated G-Rex100MCS at the following parameters: Incubated G-Rex flask: Temperature LED Display: 37.0±2.0° C.; CO₂ Percentage: 5.0±1.5% CO₂. Calculations: Time of incubation; lower limit=time of incubation+252 hours; upper limit=time of incubation+276 hours.

After process was complete, discarded any remaining warmed media and thawed aliquots of IL-2.

Day 11—Media Preparation. Monitored incubator. Incubator parameters: Temperature LED Display: 37.0±2.0° C.; CO₂ Percentage: 5.0±1.5% CO2.

Warmed 3×1000 mL RPMI 1640 Media (W3013112) bottles and 3×1000 mL AIM-V (W3009501) bottles in an incubator for ≥30 minutes. Removed RPMI 1640 Media from incubator. Prepared RPMI 1640 Media. Filter Media. Thawed 3×1.1 mL aliquots of IL-2 (6×106 IU/mL) (BR71424). Removed AIM-V Media from the incubator. Add IL-2 to AIM-V. Aseptically transferred a 10 L Labtainer Bag and a repeater pump transfer set into the BSC.

Prepared 10 L Labtainer media bag. Prepared Baxa pump. Prepared 10 L Labtainer media bag. Pumped media into 10 L Labtainer. Removed pumpmatic from Labtainer bag.

Mixed media. Gently massaged the bag to mix. Sample media per sample plan. Removed 20.0 mL of media and place in a 50 mL conical tube. Prepared cell count dilution tubes. In the BSC, added 4.5 mL of AIM-V Media that had been labelled with “For Cell Count Dilutions” and lot number to four 15 mL conical tubes. Transferred reagents from the BSC to 2-8° C. Prepared 1 L transfer pack. Outside of the BSC weld (per Process Note 5.11) a 1 L transfer pack to the transfer set attached to the “Complete CM2 Day 11 Media” bag prepared. Prepared feeder cell transfer pack. Incubated Complete CM2 Day 11 Media.

Day 11—TIL Harvest. Preprocessing table. Incubator parameters: Temperature LED display: 37.0±2.0° C.; CO₂ Percentage: 5.0±1.5% CO₂. Removed G-Rex100MCS from incubator. Prepared 300 mL Transfer Pack. Welded transfer packs to G-Rex100MCS.

Prepare flask for TIL Harvest and initiation of TIL Harvest. TIL Harvested. Using the GatheRex, transferred the cell suspension through the blood filter into the 300 mL transfer pack. Inspect membrane for adherent cells.

Rinsed flask membrane. Closed clamps on G-Rex100MCS. Ensured all clamps are closed. Heat sealed the TIL and the “Supernatant” transfer pack. Calculated volume of TIL suspension. Prepared Supernatant Transfer Pack for Sampling.

Pulled Bac-T Sample. In the BSC, draw up approximately 20.0 mL of supernatant from the 1 L “Supernatant” transfer pack and dispense into a sterile 50 mL conical tube.

Inoculated BacT per Sample Plan. Removed a 1.0 mL sample from the 50 mL conical labeled BacT prepared using an appropriately sized syringe and inoculated the anaerobic bottle.

Incubated TIL. Placed TIL transfer pack in incubator until needed. Performed cell counts and calculations. Determined the Average of Viable Cell Concentration and Viability of the cell counts performed. Viability÷2. Viable Cell Concentration÷2. Determined Upper and Lower Limit for counts. Lower Limit: Average of Viable Cell Concentration×0.9. Upper Limit: Average of Viable Cell Concentration×1.1. Confirmed both counts within acceptable limits. Determined an average viable cell concentration from all four counts performed.

Adjusted Volume of TIL Suspension: Calculate the adjusted volume of TIL suspension after removal of cell count samples. Total TIL Cell Volume (A). Volume of Cell Count Sample Removed (4.0 mL) (B) Adjusted Total TIL Cell Volume C=A−B.

Calculated Total Viable TIL Cells. Average Viable Cell Concentration*: Total Volume; Total Viable Cells: C=A×B.

Calculation for flow cytometry: if the Total Viable TIL Cell count from was ≥4.0×10⁷, calculated the volume to obtain 1.0×10⁷ cells for the flow cytometry sample.

Total viable cells required for flow cytometry: 1.0×10⁷ cells. Volume of cells required for flow cytometry: Viable cell concentration divided by 1.0×10⁷ cells A.

Calculated the volume of TIL suspension equal to 2.0×10⁸ viable cells. As needed, calculated the excess volume of TIL cells to remove and removed excess TIL and placed TIL in incubator as needed. Calculated total excess TIL removed, as needed.

Calculated amount of CS-10 media to add to excess TIL cells with the target cell concentration for freezing is 1.0×10⁸ cells/mL. Centrifuged excess TILs, as needed. Observed conical tube and added CS-10.

Filled vials. Aliquoted 1.0 mL cell suspension, into appropriately sized cryovials. Aliquoted residual volume into appropriately sized cryovial. If volume is ≤0.5 mL, add CS10 to vial until volume is 0.5 mL.

Calculated the volume of cells required to obtain 1×10⁷ cells for cryopreservation. Removed sample for cryopreservation. Placed TIL in incubator.

Cryopreservation of sample. Observed conical tube and added CS-10 slowly and record volume of 0.5 mL of CS10 added.

Day 11—Feeder Cells. Obtained feeder cells. Obtained 3 bags of feeder cells with at least two different lot numbers from LN2 freezer. Kept cells on dry ice until ready to thaw. Prepared water bath or cryotherm. Thawed feeder cells at 37.0±2.0° C. in the water bath or cytotherm for ˜3-5 minutes or until ice has just disappeared. Removed media from incubator. Pooled thawed feeder cells. Added feeder cells to transfer pack. Dispensed the feeder cells from the syringe into the transfer pack. Mixed pooled feeder cells and labeled transfer pack.

Calculated total volume of feeder cell suspension in transfer pack. Removed cell count samples. Using a separate 3 mL syringe for each sample, pulled 4×1.0 mL cell count samples from feeder cell suspension transfer pack using the needless injection port. Aliquoted each sample into the cryovials labeled. Performed cell counts and determine multiplication factors, elected protocols and entered multiplication factors. Determined the average of viable cell concentration and viability of the cell counts performed. Determined upper and lower limit for counts and confirm within limits.

Adjusted volume of feeder cell suspension. Calculated the adjusted volume of feeder cell suspension after removal of cell count samples. Calculated total viable feeder cells. Obtained additional feeder cells as needed. Thawed additional feeder cells as needed. Placed the 4th feeder cell bag into a zip top bag and thaw in a 37.0±2.0° C. water bath or cytotherm for ˜3-5 minutes and pooled additional feeder cells. Measured volume. Measured the volume of the feeder cells in the syringe and recorded below (B). Calculated the new total volume of feeder cells. Added feeder cells to transfer pack.

Prepared dilutions as needed, adding 4.5 mL of AIM-V Media to four 15 mL conical tubes. Prepared cell counts. Using a separate 3 mL syringe for each sample, removed 4×1.0 mL cell count samples from feeder cell suspension transfer pack, using the needless injection port. Performed cell counts and calculations. Determined an average viable cell concentration from all four counts performed. Adjusted volume of feeder cell suspension and calculated the adjusted volume of feeder cell suspension after removal of cell count samples. Total feeder cell volume minus 4.0 mL removed. Calculated the volume of feeder cell suspension that was required to obtain 5×10⁹ viable feeder cells. Calculated excess feeder cell volume. Calculated the volume of excess feeder cells to remove. Removed excess feeder cells.

Using a 1.0 mL syringe and 16G needle, drew up 0.15 mL of OKT3 and added OKT3. Heat sealed the feeder cell suspension transfer pack.

Day 11 G-Rex Fill and Seed Set up G-Rex500MCS. Removed “Complete CM2 Day 11 Media”, from incubator and pumped media into G-Rex500MCS. Pumped 4.5 L of media into the G-Rex500MCS, filling to the line marked on the flask. Heat sealed and incubated flask as needed. Welded the Feeder Cell suspension transfer pack to the G-Rex500MCS. Added feeder cells to G-Rex500MCS. Heat sealed. Welded the TIL suspension transfer pack to the flask. Added TILs to G-Rex500MCS. Heat sealed. Incubated G-Rex500MCS at 37.0±2.0° C., CO2 Percentage: 5.0±1.5% CO2.

Calculated incubation window. Performed calculations to determine the proper time to remove G-Rex500MCS from incubator on Day 16. Lower limit: Time of incubation+108 hours. Upper limit: Time of incubation+132 hours.

Day 11 Excess TIL Cryopreservation. Applicable: Froze Excess TIL Vials. Verified the CRF has been set up prior to freeze. Perform cryopreservation. Transferred vials from Controlled Rate Freezer to the appropriate storage. Upon completion of freeze, transfer vials from CRF to the appropriate storage container. Transferred vials to appropriate storage. Recorded storage location in LN2.

Day 16 Media Preparation. Pre-warmed AIM-V Media. Calculated time Media was warmed for media bags 1, 2, and 3. Ensured all bags have been warmed for a duration between 12 and 24 hours. Setup 10 L Labtainer for Supernatant. Attached the larger diameter end of a fluid pump transfer set to one of the female ports of a 10 L Labtainer bag using the Luer connectors. Setup 10 L Labtainer for Supernatant and label. Setup 10 L Labtainer for Supernatant. Ensure all clamps were closed prior to removing from the BSC. NOTE: Supernatant bag was used during TIL harvest, which may be performed concurrently with media preparation.

Thawed IL-2. Thawed 5×1.1 mL aliquots of IL-2 (6×10⁶ IU/mL) (BR71424) per bag of CTS AIM V media until all ice had melted. Aliquoted 100.0 mL GlutaMax. Added IL-2 to GlutaMax. Prepared CTS AIM V media bag for formulation. Prepared CTS AIM V media bag for formulation. Stage Baxa pump. Prepared to formulate media. Pumped GlutaMax+IL-2 into bag. Monitored parameters: Temperature LED Display: 37.0±2.0° C., CO₂ Percentage: 5.0±1.5% CO₂. Warmed Complete CM4 Day 16 Media. Prepared Dilutions.

Day 16 REP Spilt. Monitored Incubator parameters: Temperature LED display: 37.0±2.0° C., CO₂ Percentage: 5.0±1.5% C02. Removed G-Rex500MCS from the incubator. Prepared and labeled 1 L transfer pack as TIL suspension and weighed 1 L.

Volume Reduction of G-Rex500MCS. Transferred ˜4.5 L of culture supernatant from the G-Rex500MCS to the 10 L Labtainer.

Prepared flask for TIL harvest. After removal of the supernatant, closed all clamps to the red line.

Initiation of TIL Harvest. Vigorously tap flask and swirl media to release cells and ensure all cells have detached.

TIL Harvest. Released all clamps leading to the TIL suspension transfer pack. Using the GatheRex transferred the cell suspension into the TIL Suspension transfer pack. NOTE: Be sure to maintain the tilted edge until all cells and media are collected. Inspected membrane for adherent cells. Rinsed flask membrane. Closed clamps on G-Rex500MCS. Heat sealed the Transfer Pack containing the TIL. Heat sealed the 10 L Labtainer containing the supernatant. Recorded weight of Transfer Pack with cell suspension and calculate the volume suspension. Prepared transfer pack for sample removal. Removed testing samples from cell supernatant.

Sterility & BacT testing sampling. Removed a 1.0 mL sample from the 15 mL conical labeled BacT prepared. Removed Cell Count Samples. In the BSC, using separate 3 mL syringes for each sample, removed 4×1.0 mL cell count samples from TIL suspension transfer pack.

Removed mycoplasma samples. Using a 3 mL syringe, removed 1.0 mL from TIL suspension transfer pack and place into 15 mL conical labeled “Mycoplasma diluent”.

Prepared transfer pack for seeding. Placed TIL in incubator. Removed cell suspension from the BSC and place in incubator until needed. Performed cell counts and calculations. Diluted cell count samples initially by adding 0.5 mL of cell suspension into 4.5 mL of AIM-V media prepared which gave a 1:10 dilution. Determined the average of viable cell concentration and viability of the cell counts performed. Determined upper and lower limit for counts. Note: dilution may be adjusted according based off the expected concentration of cells. Determined an average viable cell concentration from all four counts performed. Adjusted volume of TIL suspension. Calculated the adjusted volume of TIL suspension after removal of cell count samples. Total TIL cell volume minus 5.0 mL removed for testing.

Calculated total viable TIL cells. Calculated the total number of flasks to seed. NOTE: The maximum number of G-Rex500MCS flasks to seed was five. If the calculated number of flasks to seed exceeded five, only five were seeded using the entire volume of cell suspension available.

Calculate number of flasks for subculture. Calculated the number of media bags required in addition to the bag prepared. Prepared one 10 L bag of “CM4 Day 16 Media” for every two G-Rex-500M flask needed as calculated. Proceeded to seed the first GREX-500M flask(s) while additional media is prepared and warmed. Prepared and warmed the calculated number of additional media bags determined. Filled G-Rex500MCS. Prepared to pump media and pumped 4.5 L of media into G-Rex500MCS. Heat Sealed. Repeated fill. Incubated flask. Calculated the target volume of TIL suspension to add to the new G-Rex500MCS flasks. If the calculated number of flasks exceeds five only five will be seeded, using the entire volume of cell suspension. Prepared flasks for seeding. Removed G-Rex500MCS from the incubator. Prepared G-Rex500MCS for pumping. Closed all clamps on except large filter line. Removed TIL from incubator. Prepared cell suspension for seeding. Sterile welded (per Process Note 5.11) “TIL Suspension” transfer pack to pump inlet line. Placed TIL suspension bag on a scale.

Seeded flask with TIL suspension. Pump the volume of TIL suspension calculated into flask. Heat sealed. Filled remaining flasks.

Monitored incubator. Incubator parameters: Temperature LED Display: 37.0±2.0° C., CO Percentage: 5.0±1.5% CO₂. Incubated Flasks.

Determined the time range to remove G-Rex500MCS from incubator on Day 22.

Day 22 Wash Buffer Preparation. Prepared 10 L Labtainer bag. In BSC, attach a 4″ plasma transfer set to a 10 L Labtainer bag via luer connection. Prepared 10 L Labtainer bag. Closed all clamps before transferring out of the BSC. NOTE: Prepared one 10 L Labtainer Bag for every two G-Rex500MCS flasks to be harvested. Pumped Plasmalyte into 3000 mL bag and removed air from 3000 mL Origen bag by reversing the pump and manipulating the position of the bag. Added human albumin 25% to 3000 mL Bag. Obtain a final volume of 120.0 mL of human albumin 25%.

Prepared IL-2 diluent. Using a 10 mL syringe, removed 5.0 mL of LOVO Wash Buffer using the needleless injection port on the LOVO Wash Buffer bag. Dispensed LOVO wash buffer into a 50 mL conical tube.

CRF blank bag LOVO wash buffer aliquoted. Using a 100 mL syringe, drew up 70.0 mL of LOVO Wash Buffer from the needleless injection port.

Thawed one 1.1 mL of IL-2 (6×106 IU/mL), until all ice has melted. Added 50 μL IL-2 stock (6×10⁶ IU/mL) to the 50 mL conical tube labeled “IL-2 Diluent.”

Cryopreservation preparation. Placed 5 cryo-cassettes at 2-8° C. to precondition them for final product cryopreservation.

Prepared cell count dilutions. In the BSC, added 4.5 mL of AIM-V Media that has been labelled with lot number and “For Cell Count Dilutions” to 4 separate 15 mL conical tubes. Prepared cell counts. Labeled 4 cryovials with vial number (1-4). Kept vials under BSC to be used.

Day 22 TIL Harvest. Monitored incubator. Incubator parameters: Temperature LED display: 37±2.0° C., CO₂ Percentage: 5%1.5%. Removed G-Rex500MCS Flasks from Incubator. Prepared TIL collection bag and labeled. Sealed off extra connections. Volume Reduction: Transferred ˜4.5 L of supernatant from the G-Rex500MCS to the Supernatant bag.

Prepared flask for TIL harvest. Initiated collection of TIL. Vigorously tap flask and swirl media to release cells. Ensure all cells have detached. Initiated collection of TIL. Released all clamps leading to the TIL suspension collection bag. Perform TIL harvest. Using the GatheRex, transferred the TIL suspension into the 3000 mL collection bag. Inspect membrane for adherent cells. Rinsed flask membrane. Closed clamps on G-Rex500MCS and ensured all clamps are closed. Transferred cell suspension into LOVO source bag. Closed all clamps. Heat Sealed. Removed 4×1.0 mL Cell Counts Samples

Performed Cell Counts. Performed cell counts and calculations utilizing NC-200 and Process Note 5.14. Diluted cell count samples initially by adding 0.5 mL of cell suspension into 4.5 mL of AIM-V media prepared. This gave a 1:10 dilution. Determined the average viability, viable cell concentration, and total nucleated cell concentration of the cell counts performed. Determined upper and lower limit for counts. Determined the average viability, viable cell concentration, and total nucleated cell concentration of the cell counts performed. Weighed LOVO source bag. Calculated total viable TIL Cells. Calculated total nucleated cells.

Prepared Mycoplasma Diluent. Removed 10.0 mL from one supernatant bag via luer sample port and placed in a 15 mL conical.

Performed “TIL G-Rex Harvest” protocol and determined the final product target volume. Loaded disposable kit. Removed filtrate bag. Entered filtrate capacity. Placed filtrate container on benchtop. Attached PlasmaLyte. Verified that the PlasmaLyte was attached and observed that the PlasmaLyte is moving. Attached Source container to tubing and verified Source container was attached. Confirmed PlasmaLyte was moving.

Final Formulation and Fill. Target volume/bag calculation. Calculated volume of CS-10 and LOVO wash buffer to formulate blank bag. Prepared CRF Blank.

Calculated the volume of IL-2 to add to the Final Product. Final IL-2 Concentration desired (IU/mL)—300 IU/mL. IL-2 working stock: 6×10⁴ IU/mL. Assembled connect apparatus. Sterile welded a 4S-4M60 to a CC2 cell connection. Sterile welded the CS750 cryobags to the harness prepared. Welded CS-10 bags to spikes of the 4S-4M60. Prepared TIL with IL-2. Using an appropriately sized syringe, removed amount of IL-2 determined from the “IL-2 6×10⁴” aliquot. Labeled formulated TIL Bag. Added the formulated TIL bag to the apparatus. Added CS10. Switched syringes. Drew ˜10 mL of air into a 100 mL syringe and replaced the 60 mL syringe on the apparatus. Added CS10. Prepared CS-750 bags. Dispensed cells.

Removed air from final product bags and take retain. Once the last final product bag was filled, closed all clamps. Drew 10 mL of air into a new 100 mL syringe and replace the syringe on the apparatus. Dispensed retain into a 50 mL conical tube and label tube as “Retain” and lot number. Repeat air removal step for each bag.

Prepared final product for cryopreservation, including visual inspection. Held the cryobags on cold pack or at 2-8° C. until cryopreservation.

Removed cell count sample. Using an appropriately sized pipette, remove 2.0 mL of retain and place in a 15 mL conical tube to be used for cell counts. Performed cell counts and calculations. NOTE: Diluted only one sample to appropriate dilution to verify dilution is sufficient. Diluted additional samples to appropriate dilution factor and proceed with counts. Determined the average of viable cell concentration and viability of the cell counts performed. Determined upper and lower limit for counts. NOTE: Dilution may be adjusted according based off the expected concentration of cells. Calculated IFN-γ. Heat sealed final product bags.

Samples are labeled and collected per the exemplary sample plan in Table 82.

TABLE 82
Sample plan.

| | | Sample | |
|---|---|---|---|
| | Number of | Volume to | |
| Sample | Containers | Add to Each | Container Type |
| *Mycoplasma | 1 | 1.0 mL | 15 mL Conical |
| Endotoxin | 2 | 1.0 mL | 2 mL Cryovial |
| Gram Stain | 1 | 1.0 mL | 2 mL Cryovial |
| IFN-γ | 1 | 1.0 mL | 2 mL Cryovial |
| Flow Cytometry | 1 | 1.0 mL | 2 mL Cryovial |
| Bac-T Sterility | 2 | 1.0 mL | Bac-T Bottle |
| QC Retain | 4 | 1.0 mL | 2 mL Cryovial |
| Satellite Vials | 10 | 0.5 mL | 2 mL Cryovial |

Sterility and BacT testing. Testing Sampling. In the BSC, remove a 1.0 mL sample from the retained cell suspension collected using an appropriately sized syringe and inoculate the anaerobic bottle. Repeat the above for the aerobic bottle.

Final Product Cryopreservation. Prepared controlled rate freezer (CRF). Verified the CRF had been set up and set up CRF probes. Placed final product and samples in CRF. Determined the time needed to reach 4° C.±1.5° C. and proceed with the CRF run. CRF completed and stored. Stopped the CRF after the completion of the run. Remove cassettes and vials from CRF. Transferred cassettes and vials to vapor phase LN2 for storage. Recorded storage location.

Post-processing and analysis of final drug product included the following tests: (Day 22) Determination of CD3+ cells on Day 22 REP by flow cytometry; (Day 22) Gram staining method (GMP); (Day 22) Bacterial endotoxin test by Gel Clot LAL Assay (GMP); (Day 16) BacT Sterility Assay (GMP); (Day 16) Mycoplasma DNA detection by TD-PCR (GMP); Acceptable appearance attributes; (Day 22) BacT sterility assay (GMP) (Day 22); (Day 22) IFN-gamma assay. Other potency assay as described herein are also employed to analyze TIL products.

### Example 14: A Phase 2, Multicenter Study of Autologous Tumor Infiltrating Lymphocytes in Patients with Solid Tumors

Overview of Study Design. This example describes a prospective, open-label, multi-cohort, non-randomized, multicenter Phase 2 study evaluating ACT using TIL in combination with pembrolizumab or TIL as a single therapy, using TILs prepared as described in the present application as well as in this example.

Objectives. The primary objective is to evaluate the efficacy of autologous TIL in combination with pembrolizumab in MM, HNSCC, or NSCLC patients or TIL as a single therapy in relapsed or refractory (r/r) NSCLC patients, who had previously progressed on or after treatment with CPIs, as determined by objective response rate (ORR), using the Response Evaluation Criteria in Solid Tumors (RECIST 1.1), as assessed by Investigator.

To characterize the safety profile of TIL in combination with pembrolizumab in MM, HNSCC, and NSCLC patients or TIL as a single therapy in r/r NSCLC patients as measured by the incidence of Grade ≥3 treatment-emergent adverse events (TEAEs).

The secondary objective is to further evaluate the efficacy of autologous TIL in combination with pembrolizumab in MM, HNSCC, and NSCLC patients or TIL as a single therapy in r/r NSCLC patients using complete response (CR) rate, duration of response (DOR), disease control rate (DCR), progression-free survival (PFS) using RECIST 1.1, as assessed by Investigator, and overall survival (OS).

The study includes the following cohorts:
Cohort 1A: TIL therapy in combination with pembrolizumab in patients with Stage IIIC or Stage IV unresectable or MM with ≤3 prior lines of systemic therapy excluding immunotherapy. If previously treated, patients must have had radiographically documented progression on or after most recent therapy.
Cohort 2A: TIL therapy in combination with pembrolizumab in patients with advanced, recurrent or metastatic HNSCC (e.g., Stages T1N1-N2B, T2-4N0-N2b) with 3 prior lines of systemic therapy, excluding immunotherapy. If previously treated, patients must have had radiographically documented progression on or after most recent therapy.
Cohort 3A: TIL therapy in combination with pembrolizumab in patients with locally advanced or metastatic (Stage III-IV) NSCLC with ≤3 prior lines of systemic therapy, excluding immunotherapy. If previously treated, patients must have had radiographically documented progression on or after most recent therapy.
Cohort 3B: TIL therapy as a single agent in patients Stage III or Stage IV NSCLC who have previously received systemic therapy with CPIs (e.g., anti-PD-1/anti-PD-L1) as part of ≤3 prior lines of systemic therapy. If previously treated, patients must have had radiographically documented progression on or after most recent therapy.

Patients in Cohorts 3A and 3B (NSCLC) with oncogene-driven tumors with available effective targeted therapy must have received at least one line of targeted therapy.

All patients received autologous cryopreserved TIL therapy (with or without pembrolizumab, depending on cohort assignment), preceded by a nonmyeloablative lymphodepletion (NMA-LD) preconditioning regimen consisting of cyclophosphamide and fludarabine. Following TIL infusion, up to 6 IV interleukin-2 (IL-2) doses maximum were administered.

The following general study periods took place in all 4 cohorts, unless specified otherwise.

Screening and Tumor Resection: Up to 4 weeks (28 days) from study entry; manufacturing of the TIL Product: approximately ≤22 days from tumor resection; and treatment period, as discussed below.

Treatment Period (Cohorts 1A, 2A, and 3A): up to 2 years, including NMA-LD (7 days), TIL infusion (1 day) followed by IL-2 administrations (1 to 4 days). Patients receive a single infusion of pembrolizumab after the completion of their tumor resection for TIL production and baseline scans but before the initiation of the NMA-LD regimen. The next dose of pembrolizumab will be no earlier than following the completion of IL-2 and continue Q3W±3 days thereafter for 2 years (24 months) or until disease progression or unacceptable toxicity, whichever occurs first. The end-of-treatment (EOT) visit occurred within 30 days after the last dose of pembrolizumab. The visit could be combined with end-of-assessment (EOA) visit if applicable (e.g., pembrolizumab discontinuation occurred at disease progression or at the start of new anticancer therapy).

Treatment Period (Cohort 3B): up to 12 days, including NMA-LD (7 days), TIL, infusion (1 day) followed by IL-2 administrations (1 to 4 days). The EOT visit occurred once a patient received the last dose of TL-2. The EOT visit was performed within 30 days after treatment discontinuation and it may be combined with any scheduled visit occurring within this interval during the assessment period.

Assessment Period: began after TIL infusion on Day 0 and ends upon disease progression, with the start of a new anticancer therapy, partial withdrawal of consent to study assessments, or 5 years (Month 60), whichever occurred first. An end-of assessment (EOA) visit occurred once a patient reached disease progression or started a new anticancer therapy.

The TIL autologous therapy with the TILs prepared as described herein was comprised of the following steps:
1) Tumor resection to provide the autologous tissue that serves as the source of the TIL cellular product;
2) TIL product produced at a central Good Manufacturing Practice (GMP) facility;
3) A 7-day NMA-LD preconditioning regimen;
4) Cohorts 1A, 2A, and 3A: Patients receive a single infusion of pembrolizumab after the completion of their tumor resection for TIL production and baseline scans but before the initiation of NMA-LD regimen. The next dose of pembrolizumab will be no earlier than following the completion of IL-2 and continue Q3W±3 days thereafter.
5) Infusion of the autologous TIL product (Day 0); and
6) IV IL-2 administrations for up to 6 doses maximum.

In Cohorts 1A, 2A, and 3A, the next dose of pembrolizumab was no earlier than following the completion of IL-2 and continue Q3W±3 days thereafter for ≤2 years (24 months), or until disease progression or unacceptable toxicity, whichever occurred first.

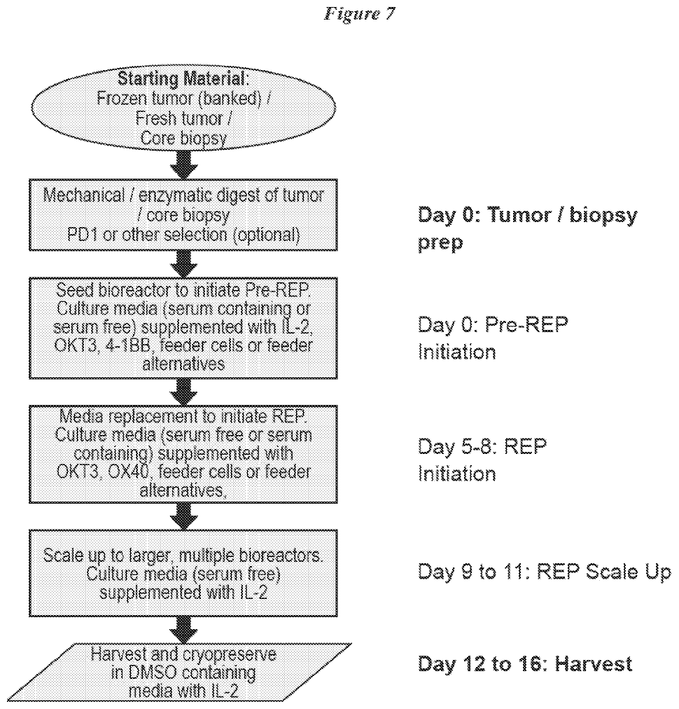

Flowcharts for Cohorts 1A, 2A, and 3A can be found in FIG. 7. The Flowchart for Cohort 3B can be found in FIG. 8. Patients were assigned to the appropriate cohort by tumor indication.

TIL Therapy+Pembrolizumab (Cohorts 1A, 2A, and 3A). Patients were screened and scheduled for surgery for tumor resection. Patients then had one or more tumor lesions resected, which were sent to a central manufacturing facility for TIL production.

Next, the NMA-LD regimen was imitated and consisted of 2 days of IV cyclophosphamide (60 mg/kg) with mesna (per site standard of care or USPI/SmPC) on Days −7 and Day −6 followed by 5 days of IV fludarabine (25 mg/m2: Day −5 through Day −1).

Patients in Cohorts 1A, 2A, and 3A received a single infusion of pembrolizumab after the completion of their tumor resection for TIL production and baseline scans and before the initiation of NMA-LD regimen. IL-2 administrations at a dose of 600,000 IU/kg IV begun as soon as 3 hours after, but no later than 24 hours after, completion of the TIL infusion on Day 0. Additional IL-2 administrations will be given approximately every 8 to 12 hours for up to 6 doses maximum. The second dose of pembrolizumab was no earlier than following the completion of IL-2. Patients should have recovered from all IL-2-related toxicities (Grade ≤2), prior to the second pembrolizumab administration. Pembrolizumab will continue Q3W±3 days thereafter for ≤2 years (24 months) or until disease progression or unacceptable toxicity, whichever occurred first.

TIL Therapy as a Single Agent (Cohort 3B). Patients were screened and scheduled for surgery for tumor resection. Patients then had one or more tumor lesions resected, which were sent to a central manufacturing facility for TIL production.

Next, the NMA-LD regimen consisted of 2 days of IV cyclophosphamide (60 mg/kg) with mesna (per site standard of care or USPI/SmPC) on Day −7 and Day −6 followed by 5 days of IV fludarabine (25 mg/m2: Day −5 through Day −1).

Infusion of the tumor-derived autologous TIL product occurred no sooner than 24 hours after last dose of fludarabine. IL-2 administrations at a dose of 600,000 IU/kg IV may have begun as soon as 3 hours after, but no later than 24 hours after, completion of the TIL infusion.

Additional IL-2 administrations were given approximately every 8 to 12 hours for up to 6 doses maximum.

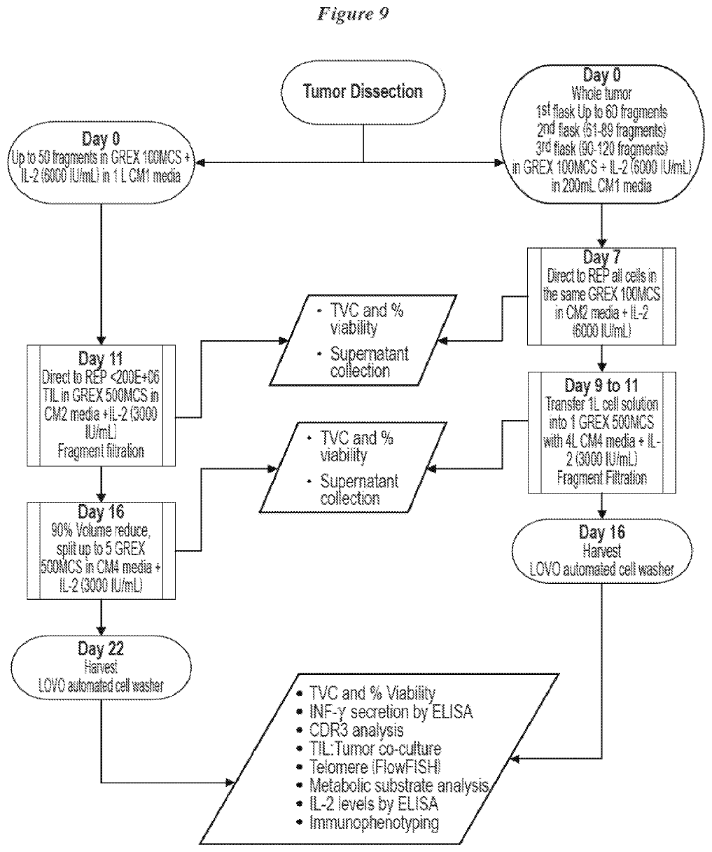

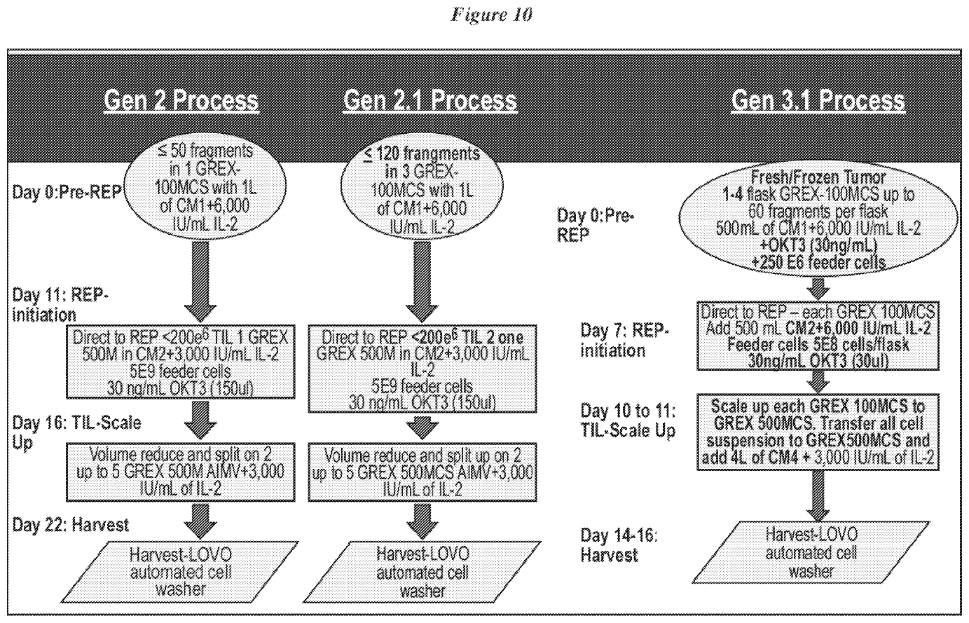

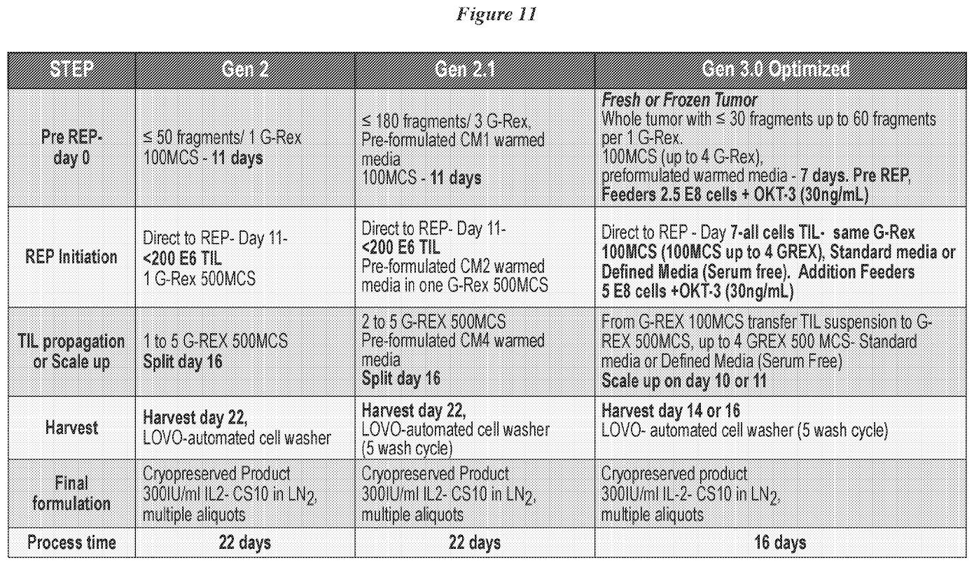

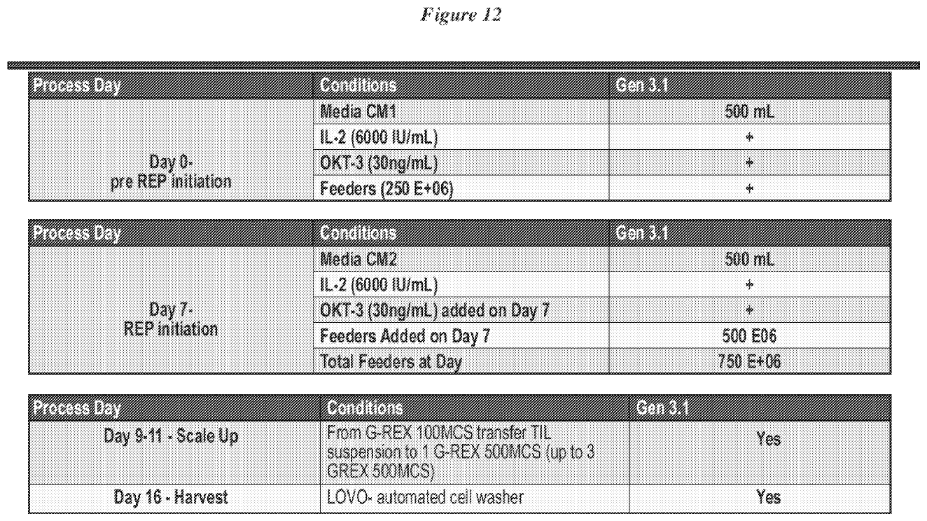

Production and Expansion of Tumor Infiltrating Lymphocytes. The TIL autologous cellular product was composed of viable cytotoxic T lymphocytes derived from a patient's tumor/lesion, which are manufactured ex vivo at a central GMP facility. An exemplary flow diagram depicting the TIL production process is provided in FIG. 9, for example.

The TIL manufacturing process begun at the clinical site after surgical excision of a primary or secondary metastatic tumor lesion(s) of ≥1.5 cm in diameter in each individual patient. Multiple tumor lesions from various anatomical locations can be excised to compile a total aggregate of tumor tissue; however, the aggregate should not exceed 4.0 cm in diameter, or 10 g in weight, due to the limited quantity of the biopreservation media present in the transport bottle.

Once the tumor lesion(s) was placed in the biopreservation transport bottle, it is shipped at 2° C. to 8° C. using an express courier to a central GMP manufacturing facility. Upon arrival, the tumor specimen(s) were dissected into fragments, which were then cultured in a pre-rapid expansion protocol (Pre-REP) with human recombinant IL-2 for ˜11 days.

These pre-REP cells were then further expanded using a rapid expansion protocol (REP) for 11 days in the presence of IL-2, OKT3 (a murine monoclonal antibody to human CD3, also known as [muromonab-CD3]) and irradiated allogeneic peripheral blood mononuclear cells (PBMC) as feeder cells.

The expanded cells were then harvested, washed, formulated, cryopreserved, and shipped to the clinical site via an express courier. The dosage form of the TIL cellular product was a cryopreserved autologous “live-cell suspension” that was ready for infusion into the patient from whom the TILs were derived. Patients were to receive the full dose of product that was manufactured and released, which contained between 1×10⁹ and 150×10⁹ viable cells per the product specification. Clinical experience indicated that objective tumor responses were achieved across this dose range, which has also been shown to be safe (Radvanyi, et al., Clin Cancer Res. 2012, 18, 6758-70). The full dose of product was provided in up to four infusion bags.

Preparation of Patients to Receive the TIL Cellular Product. The NMA-LD preconditioning regimen used in this study (i.e., 2 days of cyclophosphamide plus mesna, followed by 5 days of fludarabine) was based on the method developed and tested by the National Cancer Institute. Rosenberg, et al., Clin. Cancer Res. 2011, 17(13), 4550-7; Radvanyi, et al., Clin. Cancer Res. 2012, 18(24), 6758-70; Dudley, et al., J. Clin. Oncol. 2008, 26(32), 5233-9; Pilon-Thomas, et al., J. Immunother. 2012, 35(8), 615-20; Dudley, et al., J. Clin. Oncol. 2005, 23(10), 2346-57; and Dudley, et al., Science 2002, 298(5594), 850-4. Following the 7-day preconditioning regimen, the patient was infused with the TIL cellular product.

The TIL infusion was followed by the administration of IV IL-2 (600,000 IU/kg) every 8 to 12 hours, with the first dose administered between 3 and 24 hours after the completion of the TIL infusion and continuing for up to 6 doses maximum. Per institutional standards, the doses of IL-2 can be calculated on the basis of actual weight.

The selection of patient population for each cohort is as follows:
Cohort 1A: Patients had a confirmed diagnosis of unresectable MM (Stage IIIC or Stage IV, histologically confirmed as per American Joint Committee on Cancer [AJCC] staging system). Ocular melanoma patients were excluded. Patients must not have received prior immuno-oncology targeted agents. If BRAF-mutation positive, patient could have received prior BRAF/MEK targeted therapy.
Cohort 2A: Patients had advanced, recurrent and/or metastatic HNSCC and can be treatment naive; histologic diagnosis of the primary tumor is required via the pathology report. Patients must not have received prior immunotherapy regimens.
Cohort 3A: Patients had a confirmed diagnosis of Stage III or Stage IV NSCLC (squamous, adenocarcinoma, large cell carcinoma). Patients with oncogene-driven tumors with available effective targeted therapy had received at least one line of targeted therapy.
Cohort 3B: Patients had a confirmed diagnosis of Stage III or Stage IV NSCLC (squamous, adenocarcinoma, large cell carcinoma) and had previously received systemic therapy with CPIs (e.g., anti-PD-1/anti-PD-L1). Patients with oncogene-driven tumors with available effective targeted therapy had received at least one line of targeted therapy.

All patients had received up to 3 prior systemic anticancer therapies (see, inclusion criteria below), excluding immunotherapy for Cohorts 1A, 2A, and 3A. If previously treated, patients had radiographically confirmed progression on or after most recent therapy.

Inclusion Criteria. Patients must have met all of the following inclusion criteria for participation in the study:
1) All patients had a histologically or pathologically confirmed diagnosis of malignancy of their respective histologies:
   Unresectable or metastatic melanoma (Cohort 1A)
   Advanced, recurrent or metastatic squamous cell carcinoma of the head and neck (Cohort 2A)
   Stage III or Stage IV NSCLC (squamous, nonsquamous, adenocarcinoma, large cell carcinoma) (Cohorts 3A and 3B).
2) Cohorts 1A, 2A, and 3A only: Patients were immunotherapy naïve. If previously treated, patients had progressed on or after most recent therapy. Cohorts 1A, 2A, and 3A may have received up to 3 prior systemic anticancer therapies, specifically:
   In Cohort 1A: Patients with unresectable or metastatic melanoma (Stage IIIC or Stage IV); if BRAF mutation-positive, patients could have received a BRAF inhibitor.
   In Cohort 2A: Patients with unresectable or metastatic HNSCC. Those who had received initial chemo-radiotherapy were allowed.
   In Cohort 3A: Patients with Stage III or Stage IV NSCLC (squamous, nonsquamous, adenocarcinoma, or large cell carcinoma) and who were immunotherapy naïve and progressed after ≤3 lines of prior systemic therapy in the locally advanced or metastatic setting. Patients who received systemic therapy in the adjuvant or neoadjuvant setting, or as part of definitive chemoradiotherapy, were eligible and were considered to have had one line of therapy if the disease has progressed within 12 months of completion of prior systemic therapy. Patients with known oncogene drivers (e.g., EGFR, ALK, ROS) who had mutations that were sensitive to targeted therapies must had progressed after at least 1 line of targeted therapy.
3) Cohort 3B only: Patients with Stage III or Stage IV NSCLC (squamous, nonsquamous, adenocarcinoma, or large cell carcinoma) who had previously received systemic therapy with CPIs (e.g., anti-PD-1/anti-PD-L1) as part of ≤3 prior lines of systemic therapy.
   Patients had radiographically confirmed progression on or after most recent therapy.
   Patients who received systemic therapy in the adjuvant or neoadjuvant setting, or as part of definitive chemoradiotherapy, were eligible and were considered to have had 1 line of therapy if the disease had progressed within 12 months of completion of prior systemic therapy.
   Patients with known oncogene drivers (e.g., EGFR, ALK, ROS) who had mutations that are sensitive to targeted therapies must have progressed after at least 1 line of targeted therapy.
4) Patients had at least 1 resectable lesion (or aggregate lesions) of a minimum 1.5 cm in diameter post-resection for TIL investigational product production. It was encouraged that tumor tissue be obtained from multiple and diverse metastatic lesions, as long as the surgical resection did not pose additional risks to the patient.
   If the lesion considered for resection for TIL generation is within a previously irradiated field, the lesion must have demonstrated radiographic progression prior to resection.
   Patients must have an adequate histopathology specimen for protocol-required testing.
5) Patients had remaining measurable disease as defined by the standard RECIST 1.1 guidelines (see, for example, Eisenhauer, Eur. J Cancer 2009, 45, 228-247) following tumor resection for TIL manufacturing:
   Lesions in previously irradiated areas were not be selected as target lesions unless there had been demonstrated progression of disease in those lesions;
   Lesions that were partially resected for TIL generation that were still measurable per RECIST may be selected as nontarget lesions but could not serve as a target lesion for response assessment.
6) Patients were ≥18 years at the time of consent.
7) Patients had an Eastern Cooperative Oncology Group (ECOG) performance status of 0 or 1, and an estimated life expectancy of ≥3 months.
8) Patients of childbearing potential or those with partners of childbearing potential had to be willing to practice an approved method of highly effective birth control during treatment and continue for 12 months after receiving all protocol-related therapy (Note: Females of reproductive potential were to use effective contraception during treatment and for 12 months after their last dose of IL-2, or 4 months after their last dose of pembrolizumab whichever occurred later). Males could not donate sperm during the study or for 12 months after treatment discontinuation, whichever occurred later.
9) Patients had the following hematologic parameters:
   Absolute neutrophil count (ANC) ≥1000/mm3;
   Hemoglobin ≥29.0 g/dL;
   Platelet count ≥100,000/mm3.
10) Patients had adequate organ function:
   Serum alanine aminotransferase (ALT)/serum glutamic-pyruvic transaminase (SGPT) and aspartate aminotransferase (AST)/SGOT≤3 times the upper limit of normal (ULN), patients with liver metastasis ≤5 times ULN.
   An estimated creatinine clearance ≥40 mL/min using the Cockcroft Gault formula at Screening.
   Total bilirubin ≤2 mg/dL.
   Patients with Gilbert's Syndrome must have a total bilirubin ≤3 mg/dL.
11) Patients were seronegative for the human immunodeficiency virus (HIV1 and HIV2). Patients with positive serology for hepatitis B virus surface antigen (HBsAg), hepatitis B core antibody (anti HBc), or hepatitis C virus (anti-HCV) indicating acute or chronic infection were enrolled depending on the viral load based on polymerase chain reaction (PCR) and the local prevalence of certain viral exposures.
12) Patients had a washout period from prior anticancer therapy(ies) of a minimum duration, as detailed below prior to the first study treatment (i.e., start of NMA-LD or pembrolizumab):
   Targeted therapy: prior targeted therapy with an epidermal growth factor receptor (EGFR), MEK, BRAF, ALK, ROS1 or other-targeted agents (e.g., erlotinib, afatinib, dacomitinib, osimertinib, crizotinib, ceritinib, or lorlatinib) was allowed provided the washout is a minimum of 14 days prior to the start of treatment.
   Chemotherapy: adjuvant, neoadjuvant or definitive chemotherapy/chemoradiation was allowed provided the washout is a minimum of 21 days prior to the start of treatment.
   Immunotherapy for Cohort 3B only, prior checkpoint-targeted therapy with an anti-PD-1, other mAbs, or vaccines were allowed with a washout period of ≥21 days before the start of NMA-LD.
   Palliative radiation therapy: prior external beam radiation was allowed provided all radiation-related toxicities were resolved to Grade 1 or baseline, excluding alopecia, skin pigmentation change, or other clinically insignificant events, e.g., small area radiation dermatitis or rectal or urinary urgency.
   The tumor lesion(s) being assessed as target for response via RECIST 1.1 were outside of the radiation portal; however, if within the portal, they must have demonstrated progression (see Inclusion Criterion above).
   Surgery/pre-planned procedure: previous surgical procedure(s) was permitted provided that wound healing had occurred, all complications had resolved, and at least 14 days have elapsed (for major operative procedures) prior to the tumor resection.
13) Patients had recovered from all prior anticancer treatment-related adverse events (TRAEs) to Grade ≤1 (per Common Terminology Criteria for Adverse Events [CTCAE]), except for alopecia or vitiligo, prior to cohort assignment.
14) Patients with stable Grade ≥2 toxicity from prior anticancer therapy were considered on a case by case basis after consultation with the Medical Monitor.
15) Cohorts 1A, 2A, and 3A patients with irreversible toxicity not reasonably expected to be exacerbated by treatment with pembrolizumab were included only after consultation with the Medical Monitor. For patients in Cohort 3B only, patients with documented Grade ≥2 or higher diarrhea or colitis as a result of a previous treatment with immune checkpoint inhibitor CPI(s) must have been asymptomatic for at least 6 months or had a normal by visual assessment colonoscopy post-treatment prior to tumor resection.
16) Patients must have provided written authorization for use and disclosure of protected health information.

Exclusion Criteria. Patients who meet ANY of the following criteria were excluded from the study:
1) Patients with melanoma of uveal/ocular origin
2) Patients who had received an organ allograft or prior cell transfer therapy that included a nonmyeloablative or myeloablative chemotherapy regimen within the past 20 years. (Note: This criterion was applicable for patients undergoing retreatment with TIL, with the exception that they had a prior NMA-LD regimen with their prior TIL treatment.)
3) Patients with symptomatic and/or untreated brain metastases.
   Patients with definitively-treated brain metastases will be considered for enrollment after discussion with Medical Monitor; if, prior to the start of treatment the patient is clinically stable for ≥2 weeks, there are no new brain lesions via magnetic resonance imaging (MRI) post-treatment, and the patient does not require ongoing corticosteroid treatment.
4) Patients who are on a systemic steroid therapy within 21 days of enrollment.
5) Patients who are pregnant or breastfeeding.
6) Patients who had an active medical illness(es), which in the opinion of the Investigator, posed increased risks for study participation; such as systemic infections (e.g., syphilis or any other infection requiring antibiotics), coagulation disorders, or other active major medical illnesses of the cardiovascular, respiratory, or immune systems.
7) Patients may not have active or prior documented autoimmune or inflammatory disorders (including pneumonitis, inflammatory bowel disease (e.g., colitis or Crohn's disease), diverticulitis (with the exception of diverticulosis), systemic lupus erythematosus, sarcoidosis syndrome, or Wegener syndrome (granulomatosis with polyangiitis, Graves' disease, rheumatoid arthritis, hypophysitis, uveitis, etc.)). The following were exceptions to this criterion:
   Patients with vitiligo or alopecia.
   Patients with hypothyroidism (e.g., following Hashimoto syndrome) stable on
   hormone replacement.
   Any chronic skin condition that did not require systemic therapy.
   Patients with celiac disease controlled by diet alone.
8) Patients who had received a live or attenuated vaccination within 28 days prior to the start of treatment.
9) Patients who had any form of primary immunodeficiency (such as severe combined immunodeficiency disease [SCID] and acquired immune deficiency syndrome [AIDS]).
10) Patients with a history of hypersensitivity to any component of the study drugs. TILs were not administered to patients with a known hypersensitivity to any component of TIL product formulation including, but not limited to any of the following:
   NMA-LD (cyclophosphamide, mesna, and fludarabine)
   Proleukin®, aldesleukin, IL-2
   Antibiotics of the aminoglycoside group (i.e., streptomycin, gentamicin [excluding those who are skin-test negative for gentamicin hypersensitivity])
   Any component of the TIL product formulation including dimethyl sulfoxide [DMSO], HSA, IL-2, and dextran-40
   Pembrolizumab
11) Patients who had a left ventricular ejection fraction (LVEF) <45% or who are New York Heart Association Class II or higher. A cardiac stress test demonstrating any irreversible wall movement abnormality in any patients ≥60 years of age or in patients who have a history of ischemic heart disease, chest pain, or clinically significant atrial and/or ventricular arrhythmias.
   Patients with an abnormal cardiac stress test could be enrolled if they had adequate ejection fraction and cardiology clearance with approval of the Sponsor's Medical Monitor.
12) Patients who had obstructive or restrictive pulmonary disease and have a documented FEV1 (forced expiratory volume in 1 second) of ≤60% of predicted normal.
   If a patient was not able to perform reliable spirometry due to abnormal upper airway anatomy (i.e., tracheostomy), a 6-minute walk test was used to assess pulmonary function. Patients who were unable to walk a distance of at least 80% predicted for age and sex or demonstrates evidence of hypoxia at any point during the test (SpO2<90%) are excluded.
13) Patients who had another primary malignancy within the previous 3 years (except for those which did not require treatment or had been curatively treated greater than 1 year ago, and in the judgment of the Investigator, did not pose a significant risk of recurrence including, but not limited to, non-melanoma skin cancer, DCIS, LCIS, prostate cancer Gleason score ≤6 or bladder cancer).
14) Participation in another clinical study with an investigational product within 21 days of the initiation of treatment.

Study Endpoints and Planned Analyses. The primary and secondary endpoints were analyzed separately by cohort.

Primary Endpoints: The ORR was defined as the proportion of patients who achieved either a confirmed PR or CR as best response as assessed by Investigators per RECIST 1.1 among the efficacy analysis set.

Objective response was evaluated per each disease assessment and the ORR was expressed as a binomial proportion with the corresponding 2-sided 90% CI. The primary analysis for each cohort occurred when all treated patients per cohort have an opportunity to be followed for 12 months, unless progressed/expired or discontinued early from the assessment period.

The safety primary endpoint was measured by any Grade 3 or higher TEAE incidence rate within each cohort expressed as binomial proportions with the corresponding 2-sided 90% Cl.

Secondary Endpoints: The secondary efficacy endpoints were defined as follows:
CR rate as based on responders who achieved confirmed CR as assessed by Investigators. DCR was derived as the sum of the number of patients who achieved confirmed PR/CR or sustained SD (at least 6 weeks) divided by the number of patients in the efficacy analysis set×100%. The CR rate and DCR was summarized using a point estimate and its 2-sided 90% CI.
DOR was defined among patients who achieved objective response. It was measured from the first-time response (PR/CR) criteria are met until the first date that recurrent or progressive disease was objectively documented, or receipt of subsequent anticancer therapy or the patient dies (whichever is first recorded). Patients not experiencing PD or have not died prior to the time of data cut or the final database lock will have their event times censored on the last date that an adequate assessment of tumor status is made.
PFS was defined as the time (in months) from the time of lymphodepletion to PD, or death due to any cause, whichever event is earlier. Patients not experiencing PD or not having expired at the time of the data cut or the final database lock had their event times censored on the last date that an adequate assessment of tumor status is made.
OS was defined as the time (in months) from the time of lymphodepletion to death due to any cause. Patients not having expired by the time of data cut or the final database lock had their event times censored on the last date of their known survival status.
DOR, PFS, and OS was subjected to right censoring. The Kaplan-Meier method will be used to summarize the time-to-event efficacy endpoints. The baseline data for the tumor assessment was the last scan before the lymphodepletion for all cohorts.
The above efficacy parameters will be estimated for applicable cohort for subsets defined by baseline disease characteristics; BRAF status (Cohort 1A only), HPV status (Cohort 2A only), squamous or non-squamous lung disease (Cohorts 3A and 3B only), and anti-PD-L1 status.

### Example 15: A Phase 2, Multicenter Study of Autologous Tumor Infiltrating Lymphocytes in Patients with Locally Advanced or Metastatic Non-Small-Cell Lung Cancer

This example relates to treatment of patients with locally advanced, unresectable or metastatic non-small-cell lung cancer (NSCLC) without any actionable driver mutations who have disease progression on or following a single line of approved systemic therapy consisting of combined checkpoint inhibitor (CPI)+chemotherapy±bevacizumab (including bevacizumab (AVASTIN), a VEGFA inhibitor) and the cohorts for treatment are summarized below:
Cohort 1: Patients whose tumors did not express programmed cell death-ligand 1 (PD-L1) (tumor proportion score [TPS]<1%) prior to their CPI treatment.
Cohort 2: Patients whose tumors expressed PD-L1 (TPS≥1%) prior to their CPI treatment. Cohort 3: Patients whose tumors did not express PD-L1 (TPS<1%) prior to their CPI treatment and who are unable to safely undergo a surgical harvest for TIL generation due to at least one of the following:
   Unacceptable surgical risk, or
   Surgically approachable lesion was required for Response Evaluation Criteria in Solid Tumors (RECIST) v1.1 assessment.
Cohort 4: Retreatment cohort: Patients who had been previously treated with TIL-based immunotherapy in Cohort 1, 2 or 3 of this study.

Treatment will be given using autologous TIL-based immunotherapy derived from an individual patient's tumor for patient-directed therapy.

The TIL-based immunotherapy treatment regimen involved a course of the NMA-LD preparative regimen using cyclophosphamide and fludarabine for a total of 5 days prior to TIL-based immunotherapy infusion, and a limited course of IL-2 administration (up to six doses) following the TIL-based immunotherapy infusion. The NMA-LD preparative regimen and IL-2 were included in the regimen to support the engraftment, expansion, and activation of the transferred TILs.

Several preparative regimens had been used in conjunction with TIL therapies. NMA-LD preparative regimens included cyclophosphamide/fludarabine, total body irradiation (TBI), or the combination of both. The present exemplary study utilized the cy-flu regimen. The NMA-LD preparative regimen used in the current study was based on the method developed and tested by the National Cancer Institute (NCI), which involves 2 days of cyclophosphamide concomitant with 5 days of fludarabine in an effort to shorten the duration of the hospital stay of patients. Each patient would undergo an NMA-LD preparative regimen prior to infusion of TIL-based immunotherapy.

The therapy is a ready-to-infuse, autologous TIL-based immunotherapy. The TIL-based immunotherapy was composed of autologous TILs, which were obtained from an individual patient's tumor and expanded ex vivo through cell culture in the presence of the cytokine IL-2 and a murine monoclonal antibody (mAb) to human CD3 (OKT3).

The final drug product is a cryopreserved live-cell suspension that was formulated for IV infusion. The ex vivo expanded autologous TILs were formulated in CryoStor® CS10 cryopreservation medium/PlasmaLyte (final dimethyl sulfoxide [DMSO] concentration: 5%), with 0.5% human serum albumin (HSA) and 300 IU/mL (12 ng/mL) of IL-2. The formulated product was frozen at a controlled rate to <−150° C. in vapor phase liquid nitrogen, shipped in a cryoshipper to the appropriate clinical site, and thawed before use for infusion into the patient.

The manufacturing process began at the clinical site with the surgical resection or core biopsy of a tumor lesion containing viable tumor material. An aggregate of multiple separate lesion biopsies could also be resected from the patient and was encouraged if patient safety allows. The tumor specimen was placed in transport media and shipped by express courier at 2-8° C. to the Good Manufacturing Practices (GMP) manufacturing facility. Upon arrival at the GMP manufacturing facility, the tumor specimen was dissected into fragments, which are then activated (initial expansion step) to generate the minimum number of viable cells required for the rapid expansion protocol (REP) stage. The tumors could also be enzymatically dissociated, and TILs could be selected for expression of biomarkers prior to proceeding to the REP. The REP stage (second expansion step) further expands the cells in the presence of IL-2, OKT3, and irradiated allogeneic peripheral blood mononuclear cells (PBMC). The REP-expanded cells are then harvested, washed, and formulated in a blood transport/infusion bag for shipment by courier to the clinical site. A diagram of the manufacturing process for TIL-based immunotherapy is provided in FIGS. 34 and 35.

Each cryopreservation bag of the TIL-based immunotherapy final product was labeled with a patient-specific label. TIL-based immunotherapy was shipped from the manufacturing facility to clinical sites for administration to patients.

This example related to a prospective, open-label, multi-cohort, non-randomized, multicenter phase 2 study evaluating TIL-based immunotherapy in patients with locally advanced unresectable or metastatic NSCLC.

The following cohorts were studied:
Cohort 1: TIL-based immunotherapy as single-agent therapy in patients with Stage IV NSCLC whose tumors did not express PD-L1 (tumor proportion score [TPS]<1%) prior to their CPI treatment without a known actionable driver mutation, who had disease progression on or following a single line of approved systemic therapy consisting of combined CPI+chemotherapy±bevacizumab, who had at least one resectable lesion (or aggregate lesions) of a minimum 1.5 cm in diameter for TIL production and, following the resection, had at least one remaining measurable lesion, as defined by RECIST 1.1.
Cohort 2: TIL-based immunotherapy as single-agent therapy in patients with Stage IV NSCLC whose tumors expressed PD-L1 (TPS≥1%) prior to their CPI treatment, without any known actionable driver mutations, who had disease progression on or following a single line of approved systemic therapy consisting of combined CPI+chemotherapy±bevacizumab, and who had at least one resectable lesion (or aggregate lesions) of a minimum 1.5 cm in diameter for TIL production and, following the resection, had at least one remaining measurable lesion, as defined by RECIST 1.1.
Cohort 3: TIL-based immunotherapy as single-agent therapy in patients with Stage IV NSCLC whose tumors did not express PD-L1 (TPS<1%) prior to their CPI treatment, without any known actionable driver mutations, who had disease progression on or following a single line of approved systemic therapy consisting of combined CPI+chemotherapy±bevacizumab, and who were unable to safely undergo a surgical harvest for TIL generation due to at least one of the following: 1) unacceptable surgical risk, or 2) surgically approachable lesion is required for RECIST assessment.
Cohort 4: TIL-based immunotherapy single agent therapy as retreatment in patients who previously received TIL-based immunotherapy as part of their participation in Cohorts 1, 2 or 3.

For Cohorts 1, 2, 3, and 4, all patients received autologous cryopreserved TIL-based immunotherapy, preceded by a nonmyeloablative lymphodepletion (NMA-LD) preconditioning regimen consisting of cyclophosphamide and fludarabine. Following TIL-based immunotherapy infusion, up to 6 doses of IV IL-2 (such as aldesleukin or a biosimilar or variant thereof) were administered. Alternatively, descrescendo IL-2 or low-dose IL-2 may be used as set forth herein.

The autologous TIL therapy with TIL-based immunotherapy included the following general steps:
Tumor harvest to provide the autologous tissue that served as the source of the autologous TIL cellular product,
Production of autologous TIL-based immunotherapy investigational product (IP) at a central Good Manufacturing Practice (GMP) facility,
A 5-day nonmyeloablative lymphodepletion (NMA-LD) preconditioning regimen,
Infusion of the TIL-based immunotherapy product (Day 0), and
Administration of ≤6 doses IV IL-2.

The primary objectives are to evaluate the efficacy of TIL-based immunotherapy in patients with locally advanced unresectable or metastatic NSCLC without an actionable driver mutation who have disease progression on or following a single line of approved systemic therapy consisting of combined checkpoint inhibitor(s) (CPI[s])+chemotherapy±bevacizumab, as determined by objective response rate (ORR), using the Response Evaluation Criteria in Solid Tumors (RECIST 1.1), as assessed by the Independent Review Committee (IRC) (Cohorts 1 and 2) or by the Investigator Cohort 3 and Cohort 4).

The secondary objectives evaluated the efficacy of TIL-based immunotherapy as determined by ORR, using RECIST 1.1, and as assessed by the Investigator (Cohorts 1 and 2), further evaluated the efficacy of TIL-based immunotherapy using complete response (CR) rate; duration of response (DOR); disease control rate (DCR); progression-free survival (PFS) using RECIST 1.1, as assessed by the IRC (Cohorts 1 and 2) and Investigator (all cohorts); and overall survival (OS), and characterized the safety profile of TIL-based immunotherapy in NSCLC patients, as measured by the incidence of Grade ≥3 treatment-emergent adverse events (TEAEs). For Cohort 3 only, the efficiency of generating TIL-based immunotherapy from core biopsies is evaluated.

Exploratory Objectives: (1) Evaluated the persistence of TIL-based immunotherapy and to identify immune correlates that may affect response, outcome, and toxicity variables. (2) Assessed respective, indication-specific, health-related quality of life (HRQoL) parameters.

Primary Endpoint: ORR was assessed per RECIST 1.1 by the IRC (Cohorts 1 and 2) or by the Investigator (Cohorts 3 and 4).

Secondary Endpoints: (1) Incidence of severity, seriousness, relationship to study treatment, and characteristics of treatment-emergent adverse events (TEAEs), including serious AEs (SAEs), therapy-related AEs, and AEs leading to early discontinuation from treatment or withdrawal from the Assessment Period or death. (2) CR (complete response) rate, DOR (duration of response), DCR (disease control rate), and PFS (progression-free survival) as assessed by IRC per RECIST 1.1 (Cohorts 1 and 2). (3) ORR (objective response rate), CR rate, DOR, DCR, and PFS as assessed by the Investigator per RECIST 1.1 (all cohorts). (4) OS (overall survival). (5) Percentage successful TIL products generated from core biopsies (Cohort 3).

Exploratory Endpoints: In vivo persistence of the T cells comprising the TIL product was assessed by monitoring the presence of TIL product-specific T-cell receptor-beta complementarity determining region 3 (CDR3) sequences in the patient's blood over time. The CDR3 sequences present in the product and peripheral blood samples were identified using deep sequencing.

Exploratory endpoints aimed at identifying predictive and pharmacodynamic clinical biomarkers of the activity of TIL-based immunotherapy were also included:
Phenotypic and functional characteristics of TIL-based immunotherapy;
Immune profile of the tumor tissues;
Gene expression profiles of the TIL product, tumor tissues, and/or PBMCs;
Mutational landscape of the tumors;
Circulating immune factors; and
Immune composition of PBMC.

A HRQoL (health-related quality of life) as assessed per the European Organization for Research and Treatment of Cancer (EORTC) quality of life questionnaire (QLQ) C30 and QLQ LC13 was also included.

Study Design Details: A prospective, open-label, multi-cohort, non-randomized, multicenter phase 2 study evaluated adoptive cell therapy (ACT) with TIL-based immunotherapy.

All patients received TIL-based immunotherapy, consisting of these steps:
Tumor harvest provided the autologous tissue that serves as the source of the autologous TIL cellular product,
Production of autologous TIL-based immunotherapy investigational product (IP) at a central facility operating in accordance with Good Manufacturing Practices (GMP),
A 5-day nonmyeloablative lymphodepletion (NMA-LD) preconditioning regimen,
Infusion of the TIL-based immunotherapy product (Day 0), and
Administration of ≤6 doses IV IL-2.

The following general sequential periods will occur in all 4 cohorts, unless otherwise specified:
1. Screening Period: From informed consent form (ICF) signature to enrollment
2. Pre-treatment Period: From enrollment to initiation of preparative NMA-LD regimen.
3. Treatment Period: From initiation of preparative NMA-LD to End of Treatment (EOT) Visit. This consisted of 8 to 9 days of therapy, including NMA-LD (Days −5 to −1), TIL-based immunotherapy infusion (Day 0), followed by IL-2 administrations (Days 0 or 1 to 3 or 4). The EOT occurred approximately 30 days after Day 0.
4. Posttreatment Follow-up period, which is composed of:
   a. Posttreatment Efficacy Follow-up Period (TEFU): From EOT Visit to study completion (at 5 years [Month 60] after treatment) or the End of Efficacy Assessment (EOEA) Visit, which would be prompted by disease progression or start of a new anticancer therapy, whichever occurs first.
   b. Long-Term Follow-up Period (LTFU): From EOEA, as described above, to study completion (at 5 years [Month 60] after treatment).

Study participants (enrolled patients) will transition early to LTFU (e.g., at partial withdrawal of consent, or if is determined that they would not receive TIL-based immunotherapy for any reason). Early study withdrawal was prompted by either consent withdrawal, death, lost to follow-up, or study termination by Sponsor. A flowchart for the study design is presented in FIG. 36.

Patients will undergo a 5-day preconditioning NMA-LD regimen that was initiated prior to the planned TIL-based immunotherapy infusion on Day 0 (i.e., Days −5 through −1). The NMA LD regimen consisted of 2 days of intravenous (IV) cyclophosphamide (60 mg/kg) with mesna (per site standard of care or USPI/SmPC) on Days −5 and −4, and 5 days of fludarabine IV (25 mg/m2, Days −5 through −1).

IL-2 IV administrations at a dose of 600,000 IU/kg began as soon as 3 hours after, but no later than 24 hours after, completion of the TIL-based immunotherapy infusion on Day 0. Additional IL-2 doses were given approximately every 8 to 12 hours for up to 6 total doses.

TABLE 83
Treatment administration regimen.

| Treatment Administration | −5 | −4 | −3 | −2 | −1 | 0 | 1 | 2 | 3 | 4 |
|---|---|---|---|---|---|---|---|---|---|---|
| Cyclophosphamide 60 mg/kg | X | X | | | | | | | | |
| Mesna | X | X | | | | | | | | |
| Fludarabine 25 mg/m²/day | X | X | X | X | X | | | | | |
| TIL-based immunotherapy | | | | | | X | | | | |
| infusion | | | | | | | | | | |
| IL-2 600,000 IU/kg | | | | | | (X)^{a} | X | X | X | (X)^{a} |

^{a}() = If applicable.

Mesna Preparation: Mesna was administered to reduce the risk of hemorrhagic cystitis related to cyclophosphamide administration. Mesna was administered as a continuous or intermittent infusion as per local standards.

The total dose of mesna was not adjusted if the amount of cyclophosphamide is reduced. Dilute the volume of mesna injection or infusion per institutional standard.

Infusion of Cyclophosphamide and Mesna: Cyclophosphamide (60 mg/kg) in a total volume of 250 mL or 500 mL (e.g., 5% dextrose in water [D5W] or 0.9% sodium chloride [NaCl]). Mesna (15 mg/kg), if infused continuously, was infused over approximately 2 hours with cyclophosphamide (on Days −5 and −4), then at a rate of 3 mg/kg/hour for the remaining 22 hours in a suitable diluent over 24 hours starting concomitantly with each cyclophosphamide dose. The total dose administered was at least 1.3 times that of the dose of cyclophosphamide. Higher or continued doses of mesna could be administered for prevention of hemorrhagic cystitis.

Infusion of Fludarabine: Fludarabine (25 mg/m2) was to be given IV over approximately 30 minutes once daily for 5 consecutive days during Day −5 to Day −1.

Duration of Participation: Overall, the study participation time will be up to 5 years from treatment to completion.

### Selected Inclusion Criteria:

Had histologically or pathologically confirmed diagnosis of NSCLC (squamous, nonsquamous, adenocarcinoma, large cell, or mixed histologies), and must have documented PD-L1 expression status, as determined by the tumor proportion score (TPS) prior to the CPI treatment that they received (i.e., the historic TPS that informed the initial treatment choice) (TPS<1% for Cohorts 1 and 3, and TPS≥1% for Cohort 2).
Have received a single line of systemic therapy that included CPI and chemotherapy concurrently, with documented radiographic disease progression on or following this single line of systemic therapy.
Prior systemic therapy in the adjuvant or neoadjuvant setting, or as part of definitive chemoradiotherapy was not counted as a line of therapy if the disease had not progressed during or within 12 months of the completion of such therapy. Prior TIL treatment on this protocol did not count as a line of therapy for Cohort 4 (retreatment) patients.
Had documented exercise tolerance no less than 85% of their age-expected normal range and no signs or symptoms of ischemia or clinically significant arrhythmias.
Had Eastern Cooperative Oncology Group (ECOG) performance status of 0 or 1 and an estimated life expectancy of >6 months, in the investigator's opinion.
Cohorts 1 and 2: Must have had at least one resectable lesion (or aggregate lesions) of a minimum 1.5 cm in diameter for TIL production.
Cohort 3 only: Patients must have had a single RECIST 1.1 measurable lesion and no additional lesion available for surgical harvest, or be unable to safely undergo a surgical harvest for TIL generation, but able to safely have tumor harvest via radiology guided core biopsy sufficient for TIL generation.
Cohort 4: Followed either paradigm.
All Cohorts: If the lesion considered for harvest was within a previously irradiated field, the lesion must have demonstrated radiographic progression prior to harvest and the irradiation must have been completed at least 3 months prior to enrollment. Patients must have an adequate histopathology specimen for protocol-required testing.

Following tumor harvest for TIL manufacturing, all patients must have had at least one remaining measurable lesion, as defined by RECIST 1.1, with the following considerations:
Lesions in previously irradiated areas were not selected as target lesions unless there had been demonstrated progression in those lesions and the irradiation has been completed at least 3 months prior to enrollment.
Cohorts 1 and 2 only: Lesions that were surgically partially resected for TIL generation that were still measurable per RECIST v1.1 could be selected as nontarget lesions but could not serve as a target lesion for response assessment.
Cohort 3 only: If no other lesion was available for core biopsy for TIL generation, the single RECIST v1.1 measurable lesion may have served as both, the harvest site for the core biopsies, and the lesion for response monitoring.
Cohort 4: May follow either paradigm but must have had at least one RECIST v1.1 measurable lesion to follow for response.

The following efficacy parameters for TIL-based immunotherapy as a single therapy in patients with NSCLC were investigated in each cohort: ORR, CR, DOR, DCR, PFS, and OS.

The statistical analyses were based on the estimation of efficacy and safety parameters and will be performed by cohort. No formal statistical comparisons were applied between cohorts. The primary efficacy endpoint was ORR as assessed per RECIST v1.1 by the IRC (Cohorts 1 and 2) or by the Investigator (Cohorts 3 and 4). The ORR, CR rates, and the DCRs were summarized using point estimates and 2-sided 95% confidence limits based on the Clopper-Pearson exact method. Kaplan-Meier methods were used to summarize time-to-event efficacy endpoints, such as DOR, PFS, and OS. DOR analyses were performed for patients who achieve objective responses. The safety analyses were descriptive and based on the summarization of TEAEs, SAEs, and AEs leading to discontinuation from the study, vital signs, and clinical laboratory tests.

The total number of planned patients infused with TIL-based immunotherapy in Cohorts 1, 2 and 3 was approximately 95. For Cohort 1 and 2, approximately 40 patients were selected for each cohort. For each cohort, a Simon's two-stage design (Simon, 1989) with minimax was used to test the null hypothesis of ≤10% ORR against the alternative hypothesis of ORR>10%. In the first stage, twenty-five patients were accrued. If there are 2 or fewer patients responding to the therapy in these 25 patients, the cohort could be terminated. Otherwise, expansion into Stage 2 to a total of 40 patients occurred concurrently with the analysis of Stage 1. At the end of the second stage, if at least 7 patients respond to therapy among the total of 40 patients, the null hypothesis was rejected. This 2-stage design provided 70% power to reject the null hypothesis of 10% ORR based on an assumption of 20% ORR for TIL-based immunotherapy at a one-sided alpha level of 0.1. For Cohort 3, approximately 15 patients were planned, which provided an estimated ORR with a half-width 90% confidence interval (CI) of <0.23 by the Clopper-Pearson exact method. For Cohort 4, a retreatment cohort, patients who had been previously treated with TIL-based immunotherapy in Cohort 1, 2 or 3 of this study were enrolled.

### Example 16: Identification of Tumor Markers Suitable for Chimeric Costimulatory Receptor Design

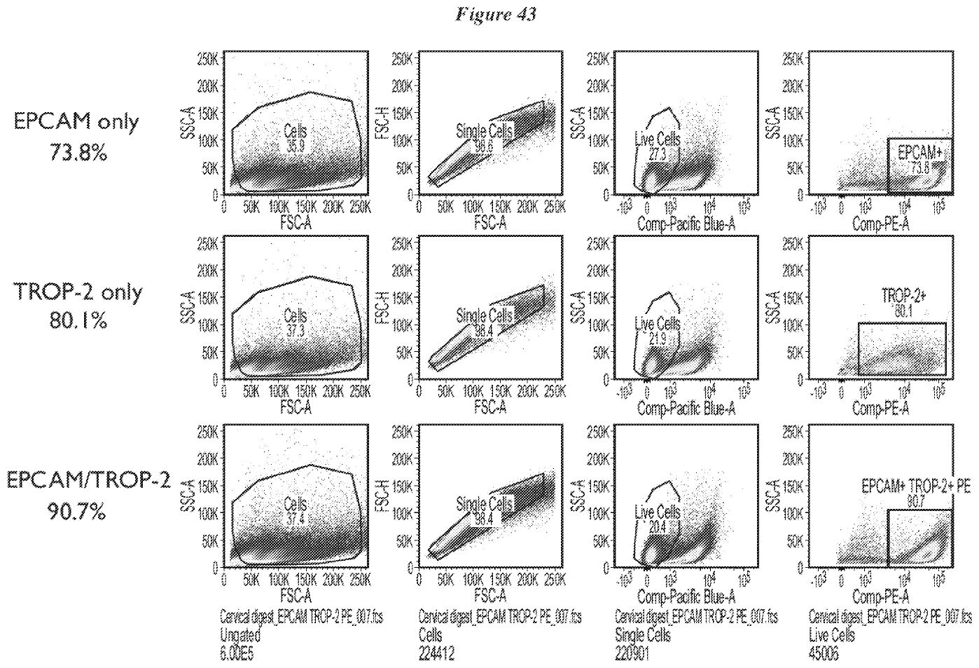

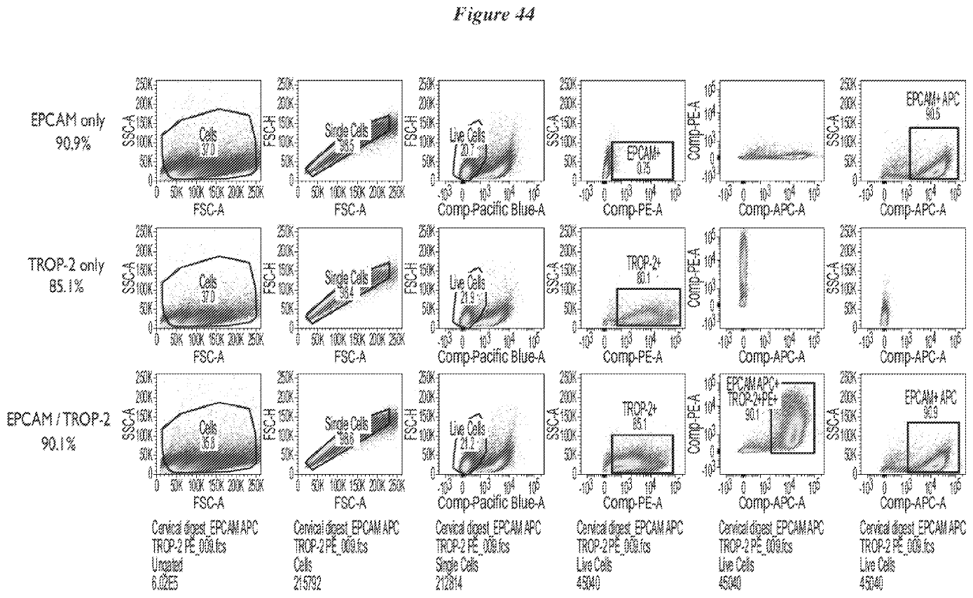

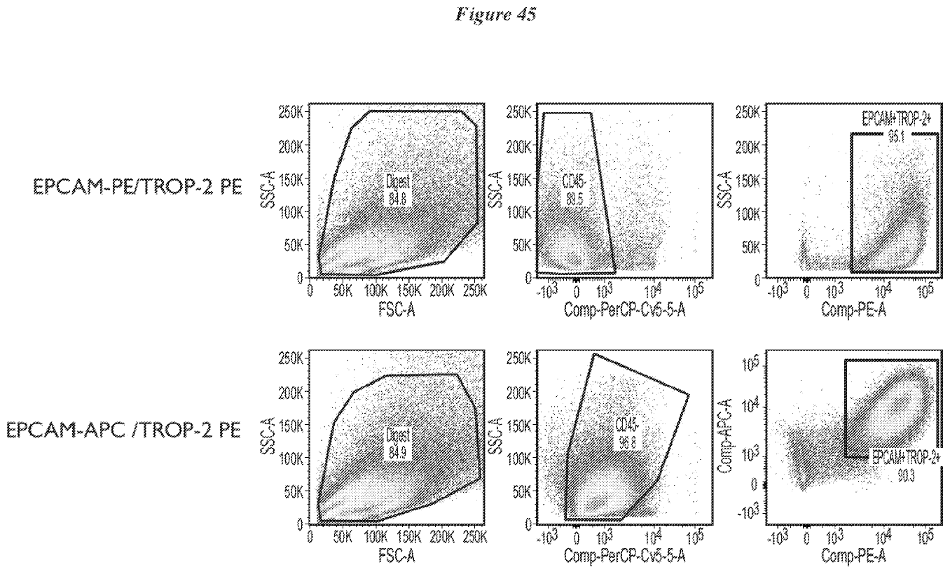

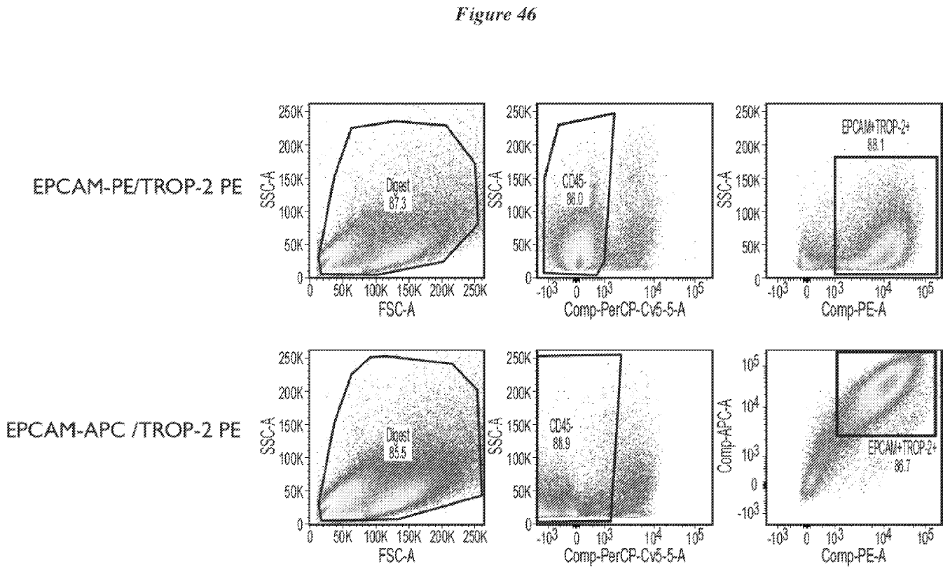

This example describes the identification of tumor markers suitable for use with CCRs and TILs modified to express CCRs. Flow cytometry analysis of tumor samples was used to measure the expression of EPCAM and TROP-2, two markers described elsewhere herein. Flow cytometry was performed using a BD Canto II system using antibodies against EPCAM, EPCAM-PE (BD, Cat. #566841) and APC (BD, Cat #566842), Clone 9C4, and an antibody against TROP-2, TROP-2 PE (BD, Cat #564837), Clone 162-46. FIG. 43 shows the flow cytometry analysis of a cervical cancer tumor (identification no. 1911271423) digest, showing high expression of EPCAM, TROP-2, and the combination of both markers. FIG. 44 similarly shows the flow cytometry analysis of a cervical cancer tumor digest, using EPCAM-APC instead of EPCAM-PE. FIG. 45 shows the flow cytometry analysis of EPCAM/TROP-2 expression on a head and neck squamous cell cancer tumor (identification no. H3103) digest. FIG. 46 shows EPCAM/TROP-2 expression on a non-small-cell lung cancer tumor (identification no. L4172) digest. Higher EPCAM and TROP-2 expression was consistently observed across multiple tumor types and replicates, showing suitability for use of a CCR targeted at these molecules in cancers described herein.

### Example 17: Preparation of TIL Products Modified with Chimeric Costimulatory Receptors

A lentiviral vector may be prepared to generate CCRs comprising an extracellular PD-1 binding domain (such as that set forth in SEQ ID NO:244, SEQ ID NO:245, or SEQ ID NO:246). Three versions of this CCR may be prepared, using the domains shown in FIG. 36, for the complete signaling domains of CD28 (YMNM+PRRP+PYAP motifs), the partial signaling domains (YMNM+PRRP motifs), and the YMNM signaling domain only (see SEQ ID NO:572).

A lentiviral vector may also be constructed to comprise the nucleotide sequences of SEQ ID NO:618, SEQ ID NO:619, or SEQ ID NO:620, corresponding to FIG. 38, FIG. 39, or FIG. 40, respectively, using cloning methods as further described herein and/or as known in the art.

For packaging, the lentiviral vectors described above may be co-transfected with a VSV-G envelope plasmid and Gag/Pol and Rev packaging plasmid into HEK293T packaging cells. After approximately two days of incubation, the supernatants are collected, centrifuged to remove debris, and filtered. Lentiviral particles are concentrated using polyethylene glycol and are further purified by sucrose gradient cushion ultracentifugation. A green fluorescence protein transgene may be added to assess transfection efficiency in research batches, before pilot and clinical batches are attempted. Transfection unit (TU) titers of at least 1×10⁹ TU/mL are desirable. Lentiviral products may be assayed for endotoxin levels and other release parameters, and subsequently used to transfect TILs, MILs, or PBLs. For example, the Gen 2 process may be modified as described elsewhere herein (for example, in FIGS. 2A, 2B, and 2C, prior to day 11 REP initiation) to transfect TILs between the pre-REP and the REP stages, optionally with a short rest period before, during, and/or after transfection, wherein the total duration of the pre-REP stage is about 3 to about 14 days, the total duration of the REP stage is about 3 to about 14 days, and a therapeutic population of TILs exceeding 10 cells is obtained, expressing the CCR, suitable for use in treatment of cancer in a patient from which the tumor was obtained.

The foregoing process may also be performed using retroviral and transposase expression systems as described elsewhere herein or as known in the art.

### Example 18: Chemokine Receptor Expression on TILs

To evaluate chemokine receptor expression on TILs, nine different TIL lines produced by the Gen 2 process were thawed and stained for characterization on two different days. PBMCs were used as controls. The TIL lots used and results are summarized in Table 84.

TABLE 84
Evaluation of chemokine receptor expression on nine TIL lots prepared from different
tumors (OV = ovarian; EP = breast; L = lung).

| Sample # | TIL Lots | Location | Live Cell Count | % Viability | Total Cell # | Vol. for 1 ml |
|---|---|---|---|---|---|---|
| 1 | OV8175 | T1B2R8P7 | 1.19 × 10⁷ | 90.3 | 11900000 | 84.03 |
| 2 | OV8178 | T1B3R3P2 | 8.18 × 10⁶ | 90.5 | 8180000 | 122.25 |
| 3 | OV8185 | T1B6R4P2 | 1.08 × 10⁷ | 88.1 | 10800000 | 92.59 |
| 4 | EP11143 | T1B5R7P3 | 7.41 × 10⁶ | 82.6 | 7410000 | 134.95 |
| 5 | EP11145 | T1B7R1P8 | 9.97 × 10⁶ | 87.6 | 9970000 | 100.30 |
| 6 | EP11147 | T4B3R7P6 | 9.47 × 10⁶ | 83.5 | 9470000 | 105.60 |
| 7 | L4231 | T1B3R6P8 | 9.54 × 10⁶ | 67.7 | 9540000 | 104.82 |
| 8 | L4233 | T1B4R6P9 | 7.58 × 10⁶ | 78.7 | 1.52 × 10⁷ | 131.93 |
| 9 | L4240 | T1B9R5P2 | 5.68 × 10⁶ | 81.9 | 5680000 | 176.06 |
| 10 | PBMC | T7B5R3P2 | 8.39 × 10⁶ | 93.3 | 16780000 | 119.19 |

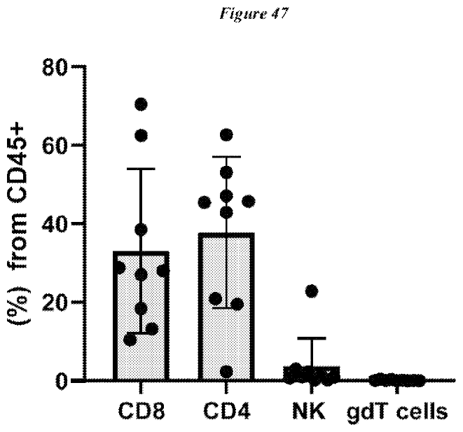

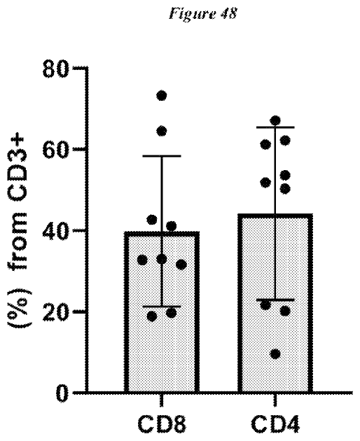

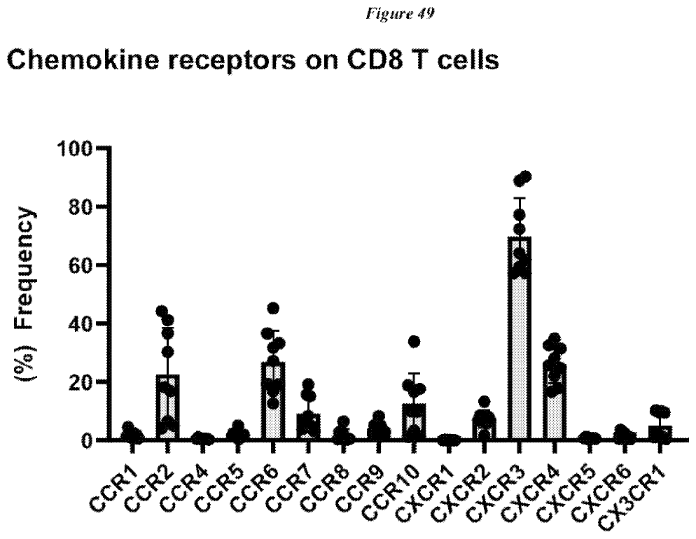

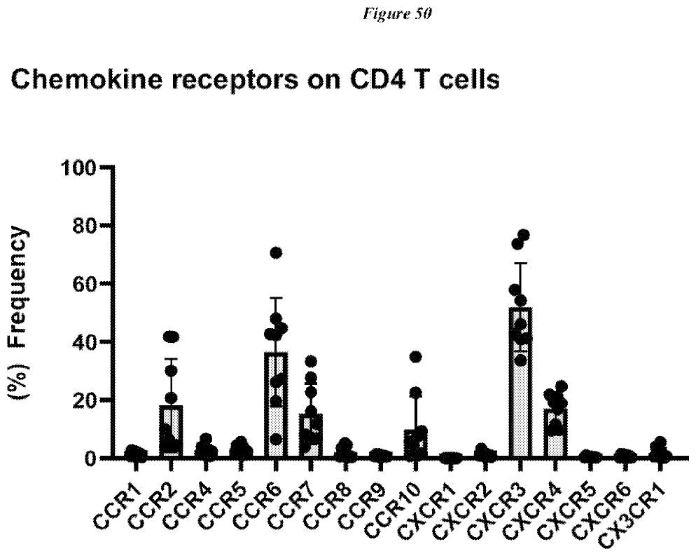

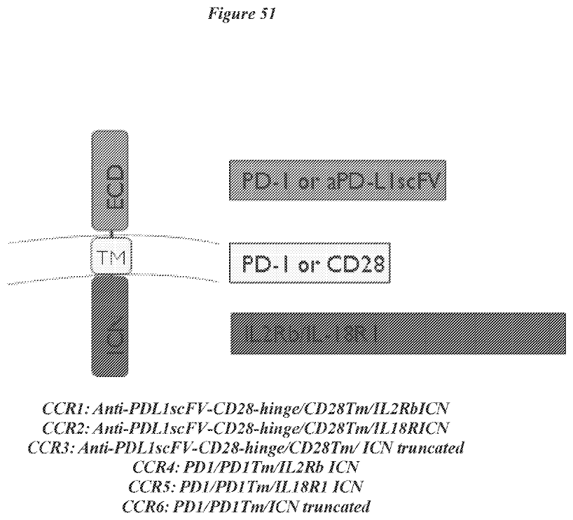

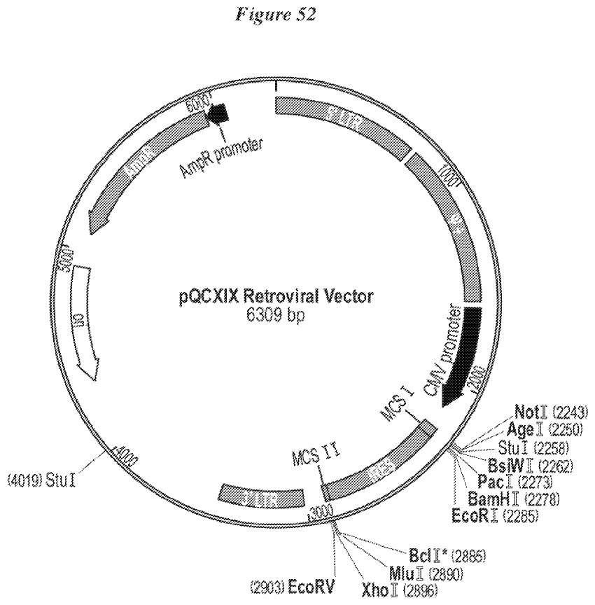

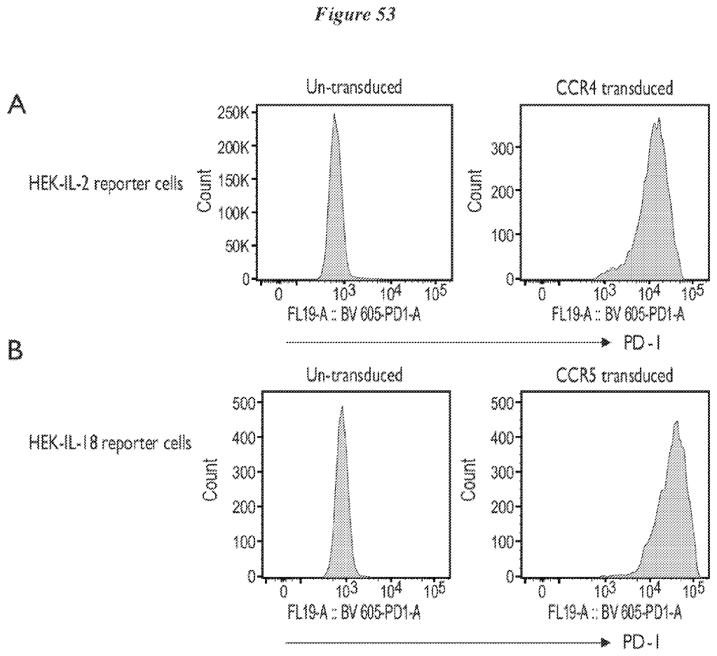

For flow cytometry analysis, a Bio-Rad ZE5 Cell Analyzer was used (Hercules, CA, USA). For surface staining of chemokine receptors, 1×10⁶ cells were plated in a 96-well V-bottom plate. Cells were then washed and stained with live/dead fixable blue dead cell stain kit from Life Technologies (Carlsbad, CA, USA) in the presence of human TruStain FcX Fc receptor blocking solution from BioLegend (San Diego, CA, USA) for 10 minutes at room temperature. Antibodies were then added to the cells and samples were incubated at 4° C. for 25 minutes. Samples were then washed twice, filtered and run on the ZE5 analyzer. Analysis was performed using FlowJo software. All gatings were based on fluorescence minus one (FMO) controls. Results are shown in FIGS. 47, 48, 49 and 50. Moderate levels of CXCR3, which binds to CXCL9/10/11, are observed. CXCR4 and CXCR5, which bind to CXCL12 and CXCL13, respectively, may be used for positioning of TILs in proximity to B cells and tertiary lymphoid structures. CXCR1 and CXCR2 bind CXCL8 amongst other ligands and may be used to co-opt trafficking of neutrophils. CCR2 binds MCP-1 and other ligands and may be used to co-opt trafficking of monocytes. CCR4 and CCR8 bind CCL17, CCL22 (CCR4) and CCL1 (CCR8), and may be used to co-opt trafficking of Tregs into tumors.

### Example 19: Preparation of TIL Products Modified with Chemokine Receptors

A VSV-G pseudotyped MSCV retroviral vector may be prepared to generate chemokine receptors comprising a CXCR1 or a CCR8 domain (such as those set forth in SEQ ID NO:627 and SEQ ID NO:632, respectively). For packaging, the vectors described above may be co-transfected with a VSV-G envelope plasmid and Gag/Pol and Rev packaging plasmid into HEK293T packaging cells. After approximately two days of incubation, the supernatants are collected, centrifuged to remove debris, and filtered. Viral particles are concentrated using polyethylene glycol and are further purified by sucrose gradient cushion ultracentifugation. A green fluorescence protein transgene may be added to assess transfection efficiency in research batches, before pilot and clinical batches are attempted. TU titers of at least 1×10⁷ TU/mL are desirable. Retroviral products may be assayed for endotoxin levels and other release parameters, and subsequently used to transfect TILs, MILs, or PBLs. For example, the Gen 2 process may be modified as described elsewhere herein (for example, in FIGS. 2A, 2B, and 2C, prior to day 11 REP initiation) to transfect TILs between the pre-REP and the REP stages, optionally with a short rest period before, during, and/or after transfection, wherein the total duration of the pre-REP stage is about 3 to about 14 days, the total duration of the REP stage is about 3 to about 14 days, and a therapeutic population of TILs exceeding 10⁸ cells is obtained, expressing one or more chemokine receptors based on the transgene(s), suitable for use in treatment of cancer in a patient from which the tumor was obtained.

Exemplary CCR constructs can be used with the TILs produced from the experiments described above. The sequences for the constructs labeled “CCR1” through “CCR6” (FIG. 51) were synthesized and inserted into the pQCXIX vector (FIG. 52) by insertion into MCS I, and eGFP was inserted into MCS II as an expression reporter. The commercially available pQCXIX vector is a self-inactivating lentiviral vector with gene expression driven by an CMV promoter. To make retrovirus, the pQCXIX vector was transfected into PT67 packing cells. Virus supernatant was collected from day 2-3 culture supernatants for transduction of HEK reporter cells. HEK reporter cells were transduced with retrovirus virus with each CCR construct. Expression of the constructs labeled “CCR4” and “CCR5” in HEK reporter cells was determined; the surface expression of the PD-1 extracellular domain in “CCR4” (FIG. 53, panel A) and “CCR5” (FIG. 53, panel B) was assayed in un-transduced (GFP−) and transduced (GFP+) HEK reporter cells by flow cytometry, with results shown in FIG. 53.

### Example 20: Biepitope Chimeric Costimulatory Receptors

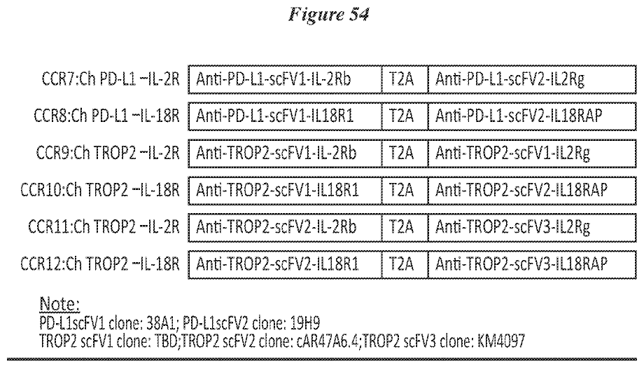

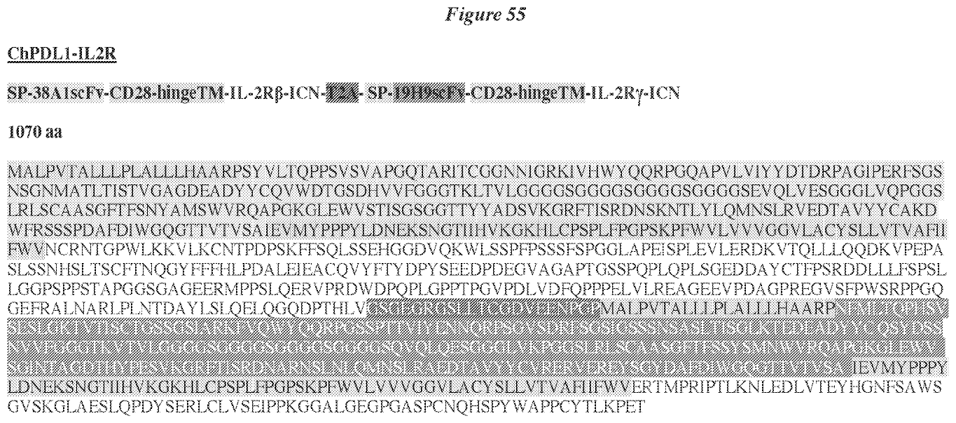

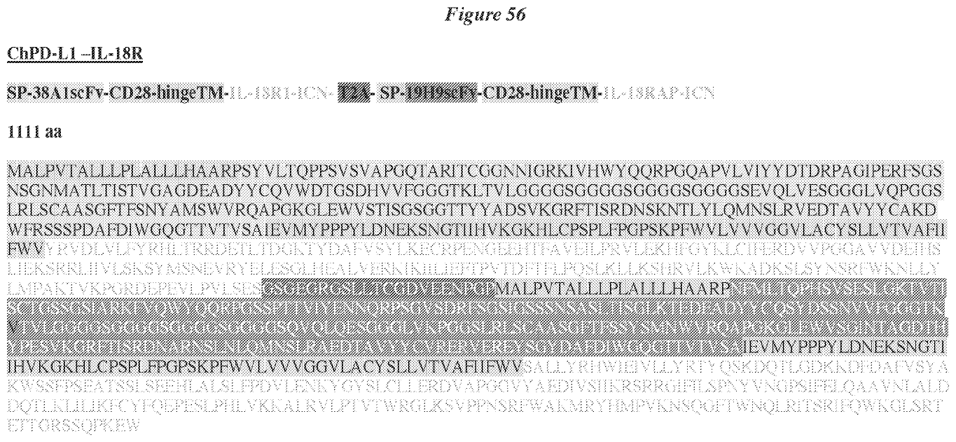

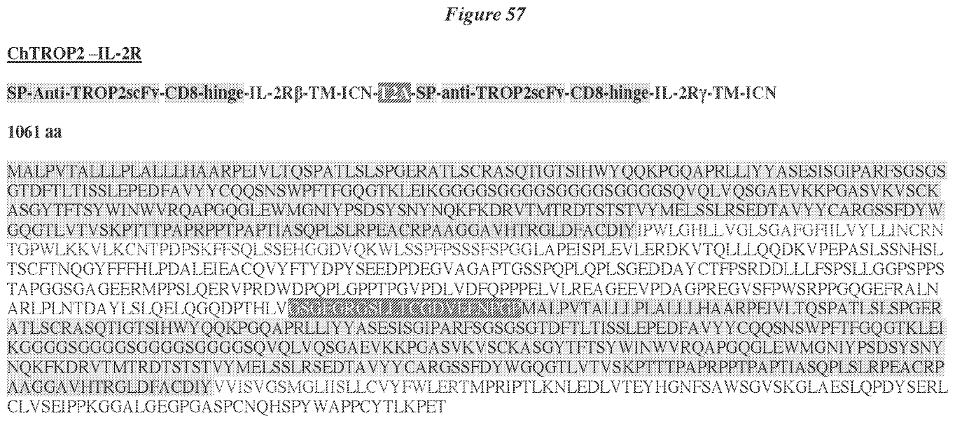

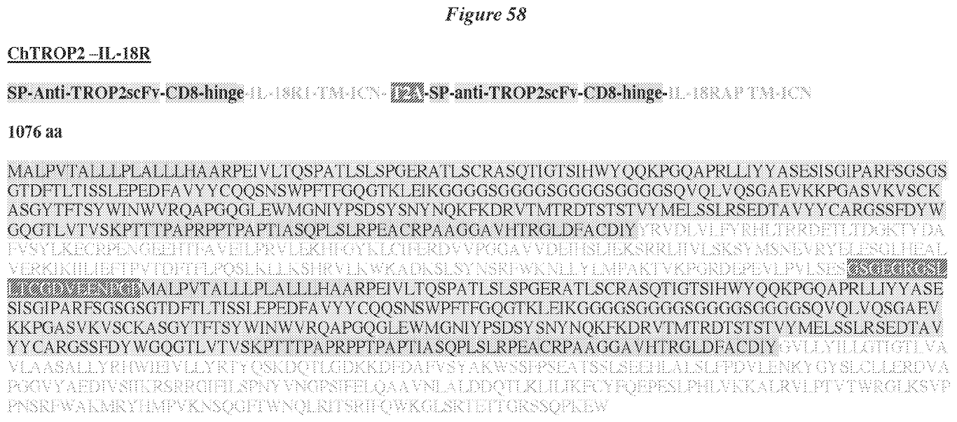

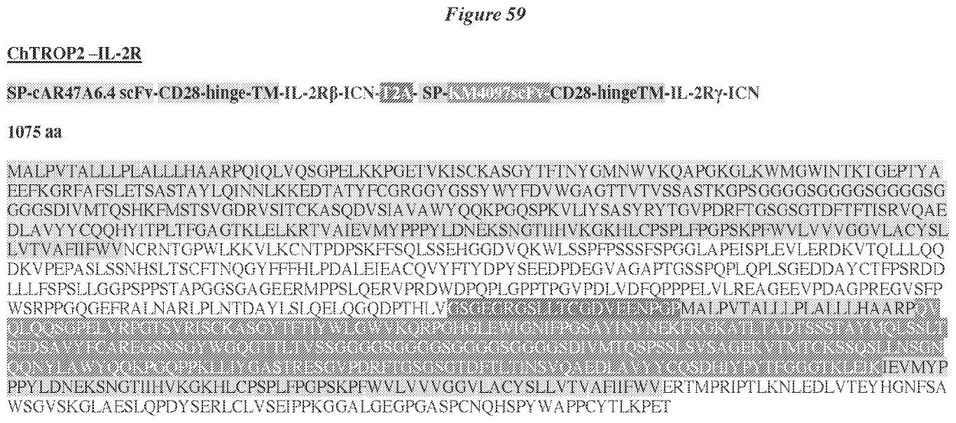

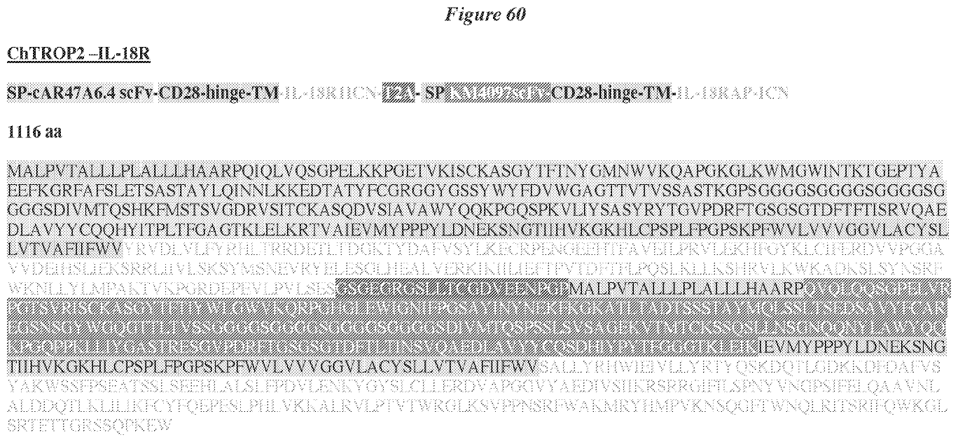

Using the methods described above, additional CCRs may be prepared. FIG. 54 shows constructs for biepitope CCRs, each designed to include two CCRs in bicistronic constructs separated by a T2A domain. The amino acid sequences with labeled domains for these CCRs are given in FIGS. 55 to 60 and Table 85. A CD8α leader peptide was used in all of the constructs. For the PD-L1 CCRs, the 19H9 and 38A1 scFv domains were selected to potentially target two epitopes of PD-L1 (SEQ ID NO:658 and SEQ ID NO:659).

For the anti-TROP-2 CCR constructs, two versions were designed. TROP-2 may potentially form a dimer, and while not being bound by theory, it is believed that one scFv (h6G11, SEQ ID NO:316) instead of two scFvs may be sufficient to pull distinct intracellular subunits closed to form a complex and transmit downstream activation signals. This feature was therefore included in the design of SEQ ID NO:660 and SEQ ID NO:661. However, if TROP-2 does not form a dimer, a two scFv strategy was included as well using clones cAR47A6.4 and KM4097 (SEQ ID NO:662 and SEQ ID NO:663), which do not compete in competition assays. Suitable, non-limiting embodiments of CCRs prepared according to this example and useful as CCR constructs of the present invention are set forth in Table 85.

TABLE 85
Amino acid sequences of exemplary biepitope CCRs.

| Identifier | Sequence (One-Letter Amino Acid Symbols) | |
|---|---|---|
| SEQ ID NO: 671 | MALPVTALLL PLALLLHAAR PSYVLTQPPS VSVAPGQTAR ITCGGNNIGR KIVHWYQQRP | 60 |
| SP-38A1scFV- | GQAPVLVIYY DTDRPAGIPE RFSGSNSGNM ATLTISTVGA GDEADYYCQV WDTGSDHVVF | 120 |
| CD28hinge-TM- | GGGTKLTVLG GGGSGGGGSG GGGSGGGGSE VQLVESGGGL VQPGGSLRLS CAASGFTFSN | 180 |
| IL-2Rb-ICN-T2A- | YAMSWVRQAP GKGLEWVSTI SGSGGTTYYA DSVKGRFTIS RDNSKNTLYL QMNSLRVEDT | 240 |
| SP 19H9scFv- | AVYYCAKDWF RSSSPDAFDI WGQGTTVTVS AIEVMYPPPY LDNEKSNGTI IHVKGKHLCP | 300 |
| CD28hingeTM-IL- | SPLFPGPSKP FWVLVVVGGV LACYSLLVTV AFIIFWVNCR NTGPWLKKVL KCNTPDPSKF | 360 |
| 2Rg-ICN | FSQLSSEHGG DVQKWLSSPF PSSSFSPGGL APEISPLEVL ERDKVTQLLL QQDKVPEPAS | 420 |
| | LSSNHSLTSC FTNQGYFFFH LPDALEIEAC QVYFTYDPYS EEDPDEGVAG APTGSSPQPL | 480 |
| | QPLSGEDDAY CTFPSRDDLL LFSPSLLGGP SPPSTAPGGS GAGEERMPPS LQERVPRDWD | 540 |
| | PQPLGPPTPG VPDLVDFQPP PELVLREAGE EVPDAGPREG VSFPWSRPPG QGEFRALNAR | 600 |
| | LPLNTDAYLS LQELQGQDPT HLVGSGEGRG SLLTCGDVEE NPGPMALPVT ALLLPLALLL | 660 |
| | HAARPNFMLT QPHSVSESLG KTVTISCTGS SGSIARKFVQ WYQQRPGSSP TTVIYENNQR | 720 |
| | PSGVSDRFSG SIGSSSNSAS LTISGLKTED EADYYCQSYD SSNVVFGGGT KVTVLGGGGS | 780 |
| | GGGGSGGGGS GGGGSQVQLQ ESGGGLVKPG GSLRLSCAAS GFTFSSYSMN WVRQAPGKGL | 840 |
| | EWVSGINTAG DTHYPESVKG RFTISRDNAR NSLNLQMNSL RAEDTAVYYC VRERVEREYS | 900 |
| | GYDAFDIWGQ GTTVTVSAIE VMYPPPYLDN EKSNGTIIHV KGKHLCPSPL FPGPSKPFWV | 960 |
| | LVVVGGVLAC YSLLVTVAFI IFWVERTMPR IPTLKNLEDL VTEYHGNFSA WSGVSKGLAE | 1020 |
| | SLQPDYSERL CLVSEIPPKG GALGEGPGAS PCNQHSPYWA PPCYTLKPET | 1070 |
| SEQ ID NO: 672 | MALPVTALLL PLALLLHAAR PSYVLTQPPS VSVAPGQTAR ITCGGNNIGR KIVHWYQQRP | 60 |
| SP-38A1scFv- | GQAPVLVIYY DTDRPAGIPE RFSGSNSGNM ATLTISTVGA GDEADYYCQV WDTGSDHVVF | 120 |
| CD28hinge-TM- | GGGTKLTVLG GGGSGGGGSG GGGSGGGGSE VQLVESGGGL VQPGGSLRLS CAASGFTFSN | 180 |
| IL-18R1-ICN- | YAMSWVRQAP GKGLEWVSTI SGSGGTTYYA DSVKGRFTIS RDNSKNTLYL QMNSLRVEDT | 240 |
| T2A-SP | AVYYCAKDWF RSSSPDAFDI WGQGTTVTVS AIEVMYPPPY LDNEKSNGTI IHVKGKHLCP | 300 |
| 19H9scFv- | SPLFPGPSKP FWVLVVVGGV LACYSLLVTV AFIIFWVYRV DLVLFYRHLT RRDETLTDGK | 360 |
| CD28hingeTM-IL- | TYDAFVSYLK ECRPENGEEH TFAVEILPRV LEKHFGYKLC IFERDVVPGG AVVDEIHSLI | 420 |
| 18RAP-ICN | EKSRRLIIVL SKSYMSNEVR YELESGLHEA LVERKIKIIL IEFTPVTDFT FLPQSLKLLK | 480 |
| | SHRVLKWKAD KSLSYNSRFW KNLLYLMPAK TVKPGRDEPE VLPVLSESGS GEGRGSLLTC | 540 |
| | GDVEENPGPM ALPVZALLLP LALLLHAARP NFMLTQPHSV SESLGKTVTI SCTGSSGSIA | 600 |
| | RKFVQWYQQR PGSSPTTVIY ENNQRPSGVS DRFSGSIGSS SNSASLTISG LKTEDEADYY | 660 |
| | CQSYDSSNVV FGGGTKVTVL GGGGSGGGGS GGGGSGGGGS QVQLQESGGG LVKPGGSLRL | 720 |
| | SCAASGFTFS SYSMNWVRQA PGKGLEWVSG INTAGDTHYP ESVKGRFTIS RDNARNSLNL | 780 |
| | QMNSLRAEDT AVYYCVRERV EREYSGYDAF DIWGQGTTVT VSAIEVMYPP PYLDNEKSNG | 840 |
| | TIIHVKGKHL CPSPLFPGPS KPFWVLVVVG GVLACYSLLV TVAFIIFWVS ALLYRHWIEI | 900 |
| | VLLYRTYQSK DQTLGDKKDF DAFVSYAKWS SFPSEATSSL SEEHLALSLF PDVLENKYGY | 960 |
| | SLCLLERDVA PGGVYAEDIV SIIKRSRRGI FILSPNYVNG PSIFELQAAV NLALDDQTLK | 1020 |
| | LILIKFCYFQ EPESLPHLVK KALRVLPTVT WRGLKSVPPN SRFWAKMRYH MPVKNSQGFT | 1080 |
| | WNQLRITSRI FQWKGLSRTE TTGRSSQPKE W | 1111 |
| SEQ ID NO: 673 | MALPVTALLL PLALLLHAAR PEIVLTQSPA TLSLSPGERA TLSCRASQTI GTSIHWYQQK | 60 |
| SP- | PGQAPRLLIY YASESISGIP ARFSGSGSGT DETLTISSLE PEDFAVYYCQ QSNSWPFTFG | 120 |
| Anti-TROP2scFv | QGTKLEIKGG GGSGGGGSGG GGSGGGGSQV QLVQSGAEVK KPGASVKVSC KASGYTFTSY | 180 |
| (h6G11)- | WINWVRQAPG QGLEWMGNIY PSDSYSNYNQ KFKDRVTMTR DTSTSTVYME LSSLRSEDTA | 240 |
| CD8hinge-IL- | VYYCARGSSF DYWGQGTLVT VSKPTTTPAP RPPTPAPTIA SQPLSLRPEA CRPAAGGAVH | 300 |
| 2Rb-TM-ICN- | TRGLDFACDI YIPWLGHLLV GLSGAFGFII LVYLLINCRN TGPWLKKVLK CNTPDPSKFF | 360 |
| T2A-SP- | SQLSSEHGGD VQKWLSSPFP SSSFSPGGLA PEISPLEVLE RDKVTQLLLQ QDKVPEPASL | 420 |
| anti-TROP2SCFV- | SSNHSLTSCF TNQGYFFFHL PDALEIEACQ VYFTYDPYSE EDPDEGVAGA PTGSSPQPLQ | 480 |
| CD8hinge-IL- | PLSGEDDAYC TFPSRDDLLL FSPSLLGGPS PPSTAPGGSG AGEERMPPSL QERVPRDWDP | 540 |
| 2Rg-TM-ICN | QPLGPPTPGV PDLVDFQPPP ELVLREAGEE VPDAGPREGV SFPWSRPPGQ GEFRALNARL | 600 |
| | PINTDAYLSL QELQGQDPTH LVGSGEGRGS LLTCGDVEEN PGPMALPVTA LLLPLALLLH | 660 |
| | AARPEIVLTQ SPATLSLSPG ERATLSCRAS QTIGTSIHWY QQKPGQAPRL LIYYASESIS | 720 |
| | GIPARFSGSG SGTDFTLTIS SLEPEDFAVY YCQQSNSWPF TFGQGTKLEI KGGGGSGGGG | 780 |
| | SGGGGSGGGG SQVQLVQSGA EVKKPGASVK VSCKASGYTF TSYWINWVRQ APGQGLEWMG | 840 |
| | NIYPSDSYSN YNQKFKDRVT MTRDTSTSTV YMELSSLRSE DTAVYYCARG SSFDYWGQGT | 900 |
| | LVTVSKPTTT PAPRPPTPAP TIASQPLSLR PEACRPAAGG AVHTRGLDFA CDIYVVISVG | 960 |
| | SMGLIISLLC VYFWLERTMP RIPTLKNLED LVTEYHGNFS AWSGVSKGLA ESLQPDYSER | 1020 |
| | LCLVSEIPPK GGALGEGPGA SPCNQHSPYW APPCYTLKPE T | 1061 |
| SEQ ID NO: 674 | MALPVTALLL PLALLLHAAR PEIVLTQSPA TLSLSPGERA TLSCRASQTI GTSIHWYQQK | 60 |
| SP- | PGQAPRLLIY YASESISGIP ARFSGSGSGT DFTLTISSLE PEDFAVYYCQ QSNSWPFTFG | 120 |
| Anti-TROP2scFv | QGTKLEIKGG GGSGGGGSGG GGSGGGGSQV QLVQSGAEVK KPGASVKVSC KASGYTFTSY | 180 |
| (h6G11)- | WINWVRQAPG QGLEWMGNIY PSDSYSNYNQ KFKDRVTMTR DTSTSTVYME LSSLRSEDTA | 240 |
| CD8hinge-IL- | VYYCARGSSF DYWGQGTLVT VSKPTTTPAP RPPTPAPTIA SQPLSLRPEA CRPAAGGAVH | 300 |
| 18R1-TM-ICN- | TRGLDFACDI YYRVDLVLFY RHLTRRDETL TDGKTYDAFV SYLKECRPEN GEEHTFAVEI | 360 |
| T2A-SP- | LPRVLEKHFG YKLCIFERDV VPGGAVVDEI HSLIEKSRRL IIVLSKSYMS NEVRYELESG | 420 |
| anti-TROP2scFv- | LHEALVERKI KIILIEFTPV TDFTFLPQSL KLLKSHRVLK WKADKSLSYN SRFWKNLLYL | 480 |
| CD8hinge-IL- | MPAKTVKPGR DEPEVLPVLS ESGSGEGRGS LLTCGDVEEN PGPMALPVTA LLLPLALLLH | 540 |
| 18RAP-TM-ICN | AARPEIVLTQ SPATLSLSPG ERATLSCRAS QTIGTSIHWY QQKPGQAPRL LIYYASESIS | 600 |
| | GIPARFSGSG SGTDFTLTIS SLEPEDFAVY YCQQSNSWPF TFGQGTKLEI KGGGGSGGGG | 660 |
| | SGGGGSGGGG SQVQLVQSGA EVKKPGASVK VSCKASGYTF TSYWINWVRQ APGQGLEWMG | 720 |
| | NIYPSDSYSN YNQKFKDRVT MTRDTSTSTV YMELSSLRSE DTAVYYCARG SSFDYWGQGT | 780 |
| | LVTVSKPTTT PAPRPPTPAP TIASQPLSLR PEACRPAAGG AVHTRGLDFA CDIYGVLLYI | 840 |
| | LIGTIGTLVA VLAASALLYR HWIEIVLLYR TYQSKDQTLG DKKDFDAFVS YAKWSSFPSE | 900 |
| | ATSSLSEEHL ALSLFPDVLE NKYGYSLCLL ERDVAPGGVY AEDIVSIIKR SRRGIFILSP | 960 |
| | NYVNGPSIFE LQAAVNLALD DQTLKLILIK FCYFQEPESL PHLVKKALRV LPTVTWRGLK | 1020 |
| | SVPPNSRFWA KMRYHMPVKN SQGFTWNQLR ITSRIFQWKG LSRTETTGRS SQPKEW | 1076 |
| SEQ ID NO: 675 | MALPVTALLL PLALLLHAAR PQIQLVQSGP ELKKPGETVK ISCKASGYTF TNYGMNWVKQ | 60 |
| SP-CAR47A6.4 | APGKGLKWMG WINTKTGEPT YAEEFKGRFA FSLETSASTA YLQINNLKKE DTATYFCGRG | 120 |
| scEv-CD28hinge- | GYGSSYWYFD VWGAGTTVTV SSASTKGPSG GGGSGGGGSG GGGSGGGGSD IVMTQSHKFM | 180 |
| TM-IL-2Rb-ICN- | STSVGDRVSI TCKASQDVSI AVAWYQQKPG QSPKVLIYSA SYRYTGVPDR FTGSGSGTDF | 240 |
| T2A-SP | TFTISRVQAE DLAVYYCQQH YITPLTFGAG TKLELKRTVA IEVMYPPPYL DNEKSNGTII | 300 |
| KM4097scFv- | HVKGKHLCPS PLFPGPSKPF WVLVVVGGVL ACYSLLVTVA FIIFWVNCRN TGPWLKKVLK | 360 |
| CD28hinge-TM- | CNTPDPSKFF SQLSSEHGGD VQKWLSSPFP SSSFSPGGLA PEISPLEVLE RDKVTQLLLQ | 420 |
| IL-2Rg-ICN | QDKVPEPASL SSNHSLTSCF TNQGYFFFHL PDALEIEACQ VYFTYDPYSE EDPDEGVAGA | 480 |
| | PTGSSPQPLQ PLSGEDDAYC TFPSRDDLLL FSPSLLGGPS PPSTAPGGSG AGEERMPPSL | 540 |
| | QERVPRDWDP QPLGPPTPGV PDLVDFQPPP ELVLREAGEE VPDAGPREGV SFPWSRPPGQ | 600 |
| | GEFRALNARL PLNTDAYLSL QELQGQDPTH LVGSGEGRGS LLTCGDVEEN PGPMALPVTA | 660 |
| | LLLPLALLLH AARPQVQLQQ SGPELVRPGT SVRISCKASG YTFTIYWLGW VKQRPGHGLE | 720 |
| | WIGNIFPGSA YINYNEKFKG KATLTADTSS STAYMQLSSL TSEDSAVYFC AREGSNSGYW | 780 |
| | GQGTTLTVSS GGGGSGGGGS GGGGSGGGGS DIVMTQSPSS LSVSAGEKVT MTCKSSQSLL | 840 |
| | NSGNQQNYLA WYQQKPGQPP KLLIYGASTR ESGVPDRFTG SGSGTDFTLT INSVQAEDLA | 900 |
| | VYYCQSDHIY PYTFGGGTKL EIKIEVMYPP PYLDNEKSNG TIIHVKGKHL CPSPLFPGPS | 960 |
| | KPFWVLVVVG GVLACYSLLV TVAFIIFWVE RTMPRIPTLK NLEDLVTEYH GNFSAWSGVS | 1020 |
| | KGLAESLQPD YSERLCLVSE IPPKGGALGE GPGASPCNQH SPYWAPPCYT LKPET | 1075 |
| SEQ ID NO: 676 | MALPVTALLL PLALLLHAAR PQIQLVQSGP ELKKPGETVK ISCKASGYTF TNYGMNWVKQ | 60 |
| SP-CAR47A6.4 | APGKGLKWMG WINTKTGEPT YAEEFKGRFA FSLETSASTA YLQINNLKKE DTATYFCGRG | 120 |
| scEv-CD28hinge- | GYGSSYWYFD VWGAGTTVTV SSASTKGPSG GGGSGGGGSG GGGSGGGGSD IVMTQSHKFM | 180 |
| TM-IL-18R1ICN- | STSVGDRVSI TCKASQDVSI AVAWYQQKPG QSPKVLIYSA SYRYTGVPDR FTGSGSGTDF | 240 |
| T2A-SP | TFTISRVQAE DLAVYYCQQH YITPLTFGAG TKLELKRTVA IEVMYPPPYL DNEKSNGTII | 300 |
| KM4097scFv- | HVKGKHLCPS PLFPGPSKPF WVLVVVGGVL ACYSLLVTVA FIIFWVYRVD LVLFYRHLTR | 360 |
| CD28hinge-TM- | RDETLTDGKT YDAFVSYLKE CRPENGEEHT FAVEILPRVL EKHFGYKLCI FERDVVPGGA | 420 |
| IL-18RAP-ICN | VVDEIHSLIE KSRRLIIVLS KSYMSNEVRY ELESGLHEAL VERKIKIILI EFTPVTDFTF | 480 |
| | LPQSLKLLKS HRVLKWKADK SLSYNSRFWK NLLYLMPAKT VKPGRDEPEV LPVLSESGSG | 540 |
| | EGRGSLLTCG DVEENPGPMA LPVTALLLPL ALLLHAARPQ VQLQQSGPEL VRPGTSVRIS | 600 |
| | CKASGYTFTI YWLGWVKQRP GHGLEWIGNI FPGSAYINYN EKFKGKATLT ADTSSSTAYM | 660 |
| | QLSSLTSEDS AVYFCAREGS NSGYWGQGTT LTVSSGGGGS GGGGSGGGGS GGGGSDIVMT | 720 |
| | QSPSSLSVSA GEKVEMTCKS SQSLLNSGNQ QNYLAWYQQK PGQPPKLLIY GASTRESGVP | 780 |
| | DRFTGSGSGT DFTLTINSVQ AEDLAVYYCQ SCHIYPYTFG GGTKLEIKIE VMYPPPYLDN | 840 |
| | EKSNGTIIHV KGKHLCPSPL FPGPSKPFWV LVVVGGVLAC YSLLVTVAFI IFWVSALLYR | 900 |
| | HWIEIVLLYR TYQSKDQTLG DKKDFDAFVS YAKWSSFPSE ATSSLSEEHL ALSLFPDVLE | 960 |
| | NKYGYSLCLL ERDVAPGGVY AEDIVSIIKR SRRGIFILSP NYVNGPSIFE LQAAVNLALD | 1020 |
| | DQTLKLILIK FCYFQEPESL PHLVKKALRV LPTVTWRGLK SVPPNSRFWA KMRYHMPVKN | 1080 |
| | SQGFTWNQLR ITSR_FQWKG LSRTETTGRS SQPKEW | 1116 |

The foregoing examples, which are also embodiments of the present invention, provide for the expression of the sequences of SEQ ID NO:671, SEQ ID NO:672, SEQ ID NO:673, SEQ ID NO:674, SEQ ID NO:675, or SEQ ID NO:676, or conservative amino acid substitutions thereof, or fragments, variants, or derivatives thereof, or an amino acid sequence that is at least 99% identical to the sequence given in SEQ ID NO:671, SEQ ID NO:672, SEQ ID NO:673, SEQ ID NO:674, SEQ ID NO:675, or SEQ ID NO:676, at least 98% identical to the sequence given in SEQ ID NO:671, SEQ ID NO:672, SEQ ID NO:673, SEQ ID NO:674, SEQ ID NO:675, or SEQ ID NO:676, at least 97% identical to the sequence given in SEQ ID NO:671, SEQ ID NO:672, SEQ ID NO:673, SEQ ID NO:674, SEQ ID NO:675, or SEQ ID NO:676, at least 96% identical to the sequence given in SEQ ID NO:671, SEQ ID NO:672, SEQ ID NO:673, SEQ ID NO:674, SEQ ID NO:675, or SEQ ID NO:676, at least 95% identical to the sequence given in SEQ ID NO:671, SEQ ID NO:672, SEQ ID NO:673, SEQ ID NO:674, SEQ ID NO:675, or SEQ ID NO:676, at least 90% identical to the sequence given in SEQ ID NO:671, SEQ ID NO:672, SEQ ID NO:673, SEQ ID NO:674, SEQ ID NO:675, or SEQ ID NO:676, at least 85% identical to the sequence given in SEQ ID NO:671, SEQ ID NO:672, SEQ ID NO:673, SEQ ID NO:674, SEQ ID NO:675, or SEQ ID NO:676, or at least 80% identical to the sequence given in SEQ ID NO:671, SEQ ID NO:672, SEQ ID NO:673, SEQ ID NO:674, SEQ ID NO:675, or SEQ ID NO:676.

In an embodiment, these constructs are encoded by the vectors of SEQ ID NO:621, SEQ ID NO:622, SEQ ID NO:623, SEQ ID NO:624, SEQ ID NO:625, and SEQ ID NO:626. In an embodiment, a CCR of the present invention comprises compositions comprising the foregoing biepitope CCRs expressed via one of the foregoing bicistronic constructs. The eGFP domains may be removed for use in the preparation of TILs for human therapy.

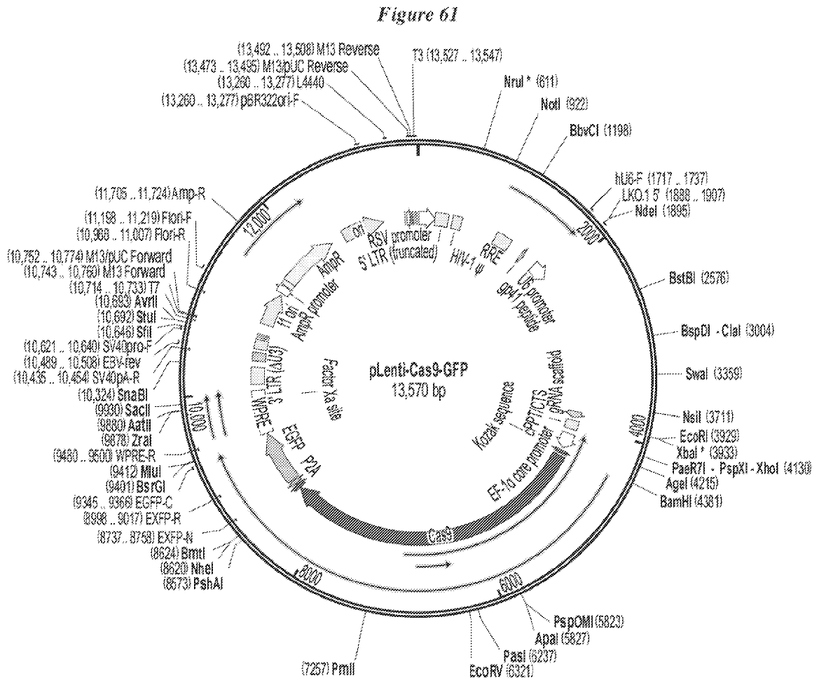

The CCR7 to CCR12 sequences (as shown in FIG. 54) were synthesized and inserted into the pLenti-virus vector (FIG. 61) by replacing the Cas9-GFP cassette for the gene of interest. The pLenti-virus vector is a self-inactivating lentiviral vector with gene expression driven by an EF-1α core promoter. To make lentivirus, pLenti vectors and helper vectors (VSV-G, Gag/Pol) were con-transfected into 293T cells. Virus supernatant was collected from day 2 to 3 culture supernatants for transduction of HEK reporter cells.

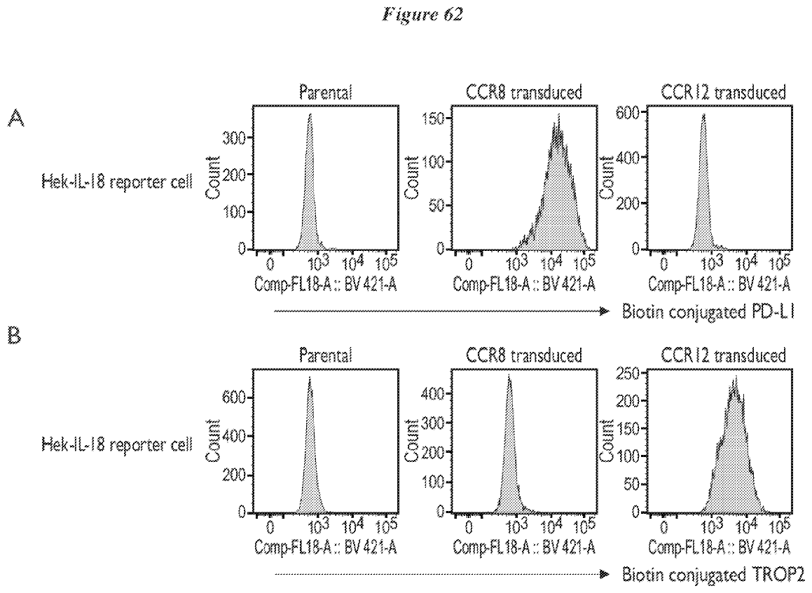

Exemplary results showing the expression of the biepitope CCR8 and CCR12 constructs in HEK reporter cells are presented in FIG. 62. HEK reporter cells were transduced with lentivirus with the indicated CCR8 and CCR12 constructs. In FIG. 62, the results for the HEK-IL-18 reporter cells transduced with CCR8 and incubated with biotin conjugated PD-L1 protein, shown in panel (A), and the results for the HEK-IL-18 reporter cells transduced with CCR12 and incubated with biotin conjugated TROP-2 protein, shown in panel (B), each after streptavidin-fluorescent staining, demonstrate expression of the desired CCRs.

Competitive binding of these antibodies was then studied in order to determine feasibility for a biepitope CCR construct. Characterization data for the anti-PD-L1 antibody clone 38A1-IgG4-HA and 19H-IgG4-Flag is given in Table 86.

TABLE 86
Characterization of anti-PD-L1 Ab clone
38A1-IgG4-HA and 19H-IgG4-Flag.

| | EC50 | Ab stock | MW | |
|---|---|---|---|---|
| 38A1-IgG4-HA | 0.4039 nM | 0.45 μg/μL | 54.162 | kDa |
| 19H9-IgG4-Flag | 0.1248 nM | 0.96 μg/μL | 55.481 | KDa |

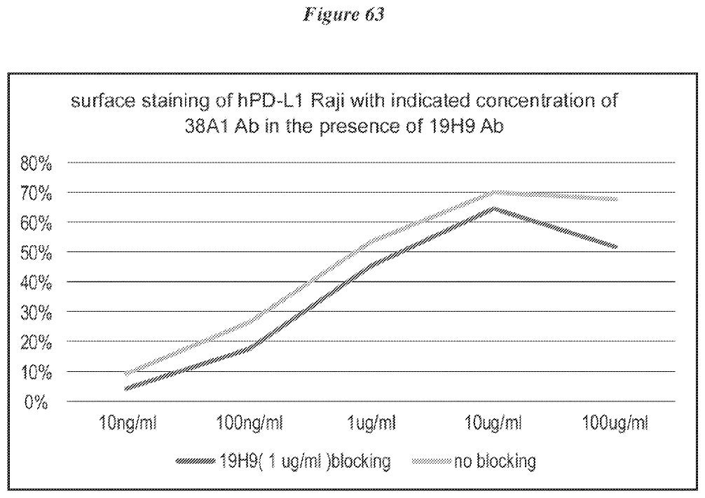

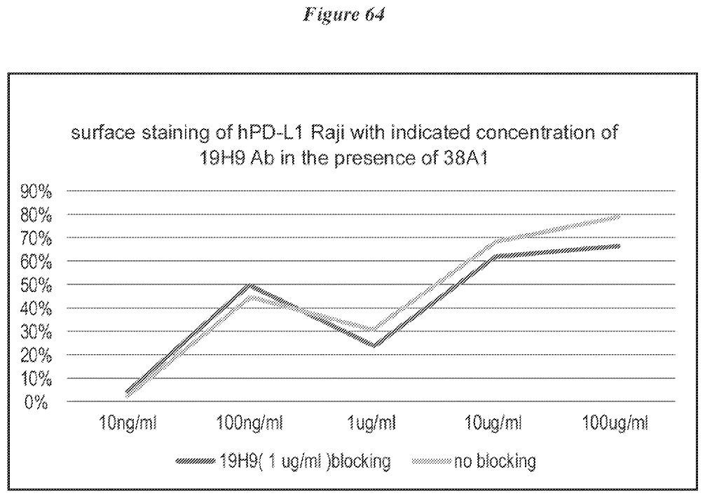

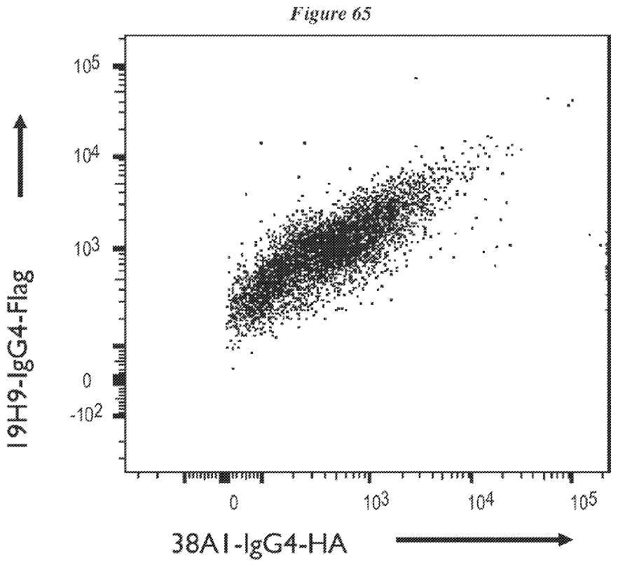

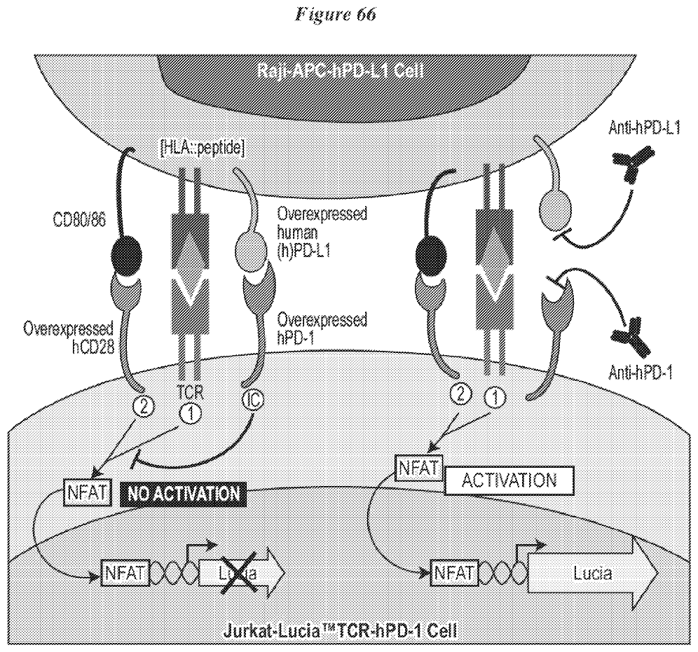

First, hPD-L1 Raji cells were incubated with indicated (in FIG. 63) concentrations of 38A1-IgG4-HA antibody targeting PD-L1 in the presence of competitive hPD-L1 binding antibody 19H9. After 2 hours of incubation, cells were washed and stained with anti-HA-APC for analysis. In FIG. 63, the x-axis shows the concentration of the titrated 38A1-IgG4-HA antibody and the y-axis shows the % PD-L1 positive staining cells of total hPD-L1 Raji cells. hPD-L1 Raji cells were then incubated with indicated (in FIG. 64) concentrations of 19H9-IgG4-Flag antibody targeting PD-L1 in the presence of competitive hPD-L1 binding antibody 19H9. After 2 hours of incubation, cells were washed and stained with anti-Flag-AF488. In FIG. 64, the x-axis shows the concentration of the titrated 19H9-IgG4-Flag antibody and the Y-axis shows % PD-L1 positive staining cells of total hPD-L1 Raji cells. The results indicate that 38A1 and 19H9 bind non-competitively to PD-L1. FIG. 65 shows co-stained flow cytometry data for the Raji cells with staining of each indicated antibody; wherein. hPDL-1-Raji cells were incubated with 19H9-IgG4-Flag Ab and 38A1-IgG4-HA Ab followed by incubation with fluorophore-conjugated secondary antibodies.

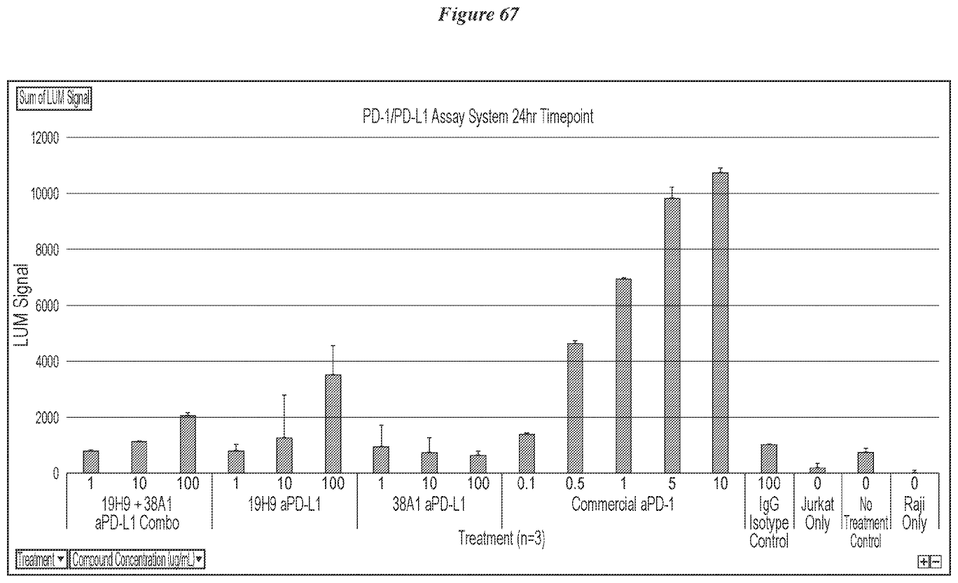

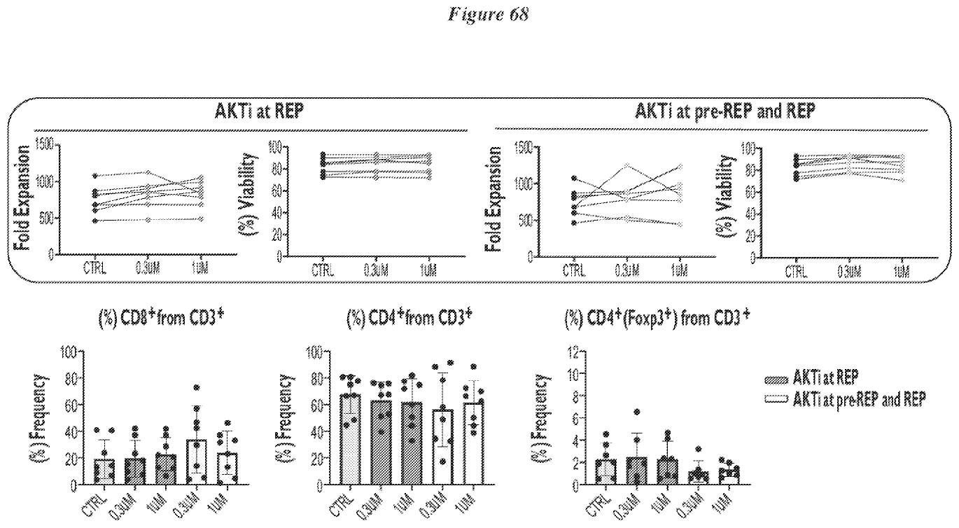

Additional experiments were performed to assess the blocking efficacy of the two PD-L1 antibodies using the approach depicted in FIG. 66. Jurkat-Lucia™ TCR-hPD-1 cells (2×10⁵ cells) and Raji-APC-hPD-L1 cell (2×10⁵ cells) were co-cultured in the presence of 19H9 and 38A1 anti-PD-L1 antibodies with the indicated concentrations shown on the x-axis of FIG. 67. 19H9 and 38A1 were added single or combined into co-culture system. After 24 hours, Lucia activity were quantified with QUANTI-Luc™ assay kit. Results are shown in FIG. 67.

In conclusion, the biepitope CCR constructs can be successfully expressed in HEK-blue reporter cells, and exhibit antigen specific binding capability. Surprisingly, there is no competitive binding of PD-L1 for the PD-L1 clones 38A1 and 19H9, indicating they bind different PD-L1 epitopes. These clones are suitable for biepitope CCR designs as shown in this example. Such designs can be capable of providing two subunits for IL-18R and IL-2R intracellular domains.

### Example 21: Use of Akt Inhibitors to Increase the CD39-CD69-Phenotype in TIL Products

A memory-progenitor stem-like (CD39⁻CD69⁻) phenotype was associated with complete regression and TIL persistence in a cohort of patients with metastatic melanoma (Krishna, et al., Science 2020, 370, 1328). Strategies aimed at expanding TIL with less differentiated and more stem-like attributes may result in improved persistence, functionality, and more effective tumor responses. Pharmacologic inhibition of AKT in TILs has been shown to induce transcriptional, metabolic, and functional properties characteristic of memory T cells. In this example, AKT inhibition during cx vivo TIL expansion was investigated to determine if it could increase the proportion of less differentiated, more stem-like cells with improved cytokine output and functionality.

Patient tumors from different indications were received, fragmented, and subjected to an expansion protocol for TIL manufacturing. Different doses (0.3 μM and 1 μM) of the pan-AKT inhibitor ipatasertib were added to the culture during ex vivo expansion. The expansion potential, as well as the phenotypic and functional characteristics were evaluated on the final TIL product. Eight tumors, including tumors from melanoma, NSCLC, head and neck cancer, ovarian cancer, and breast cancer, were used in combination with the Gen 2 process.

FIG. 68 shows that AKT inhibitor treatment maintains TIL expansion and viability without affecting T cell ratios. Expansion, viability and T cell distribution in control and AKT inhibitor-treated TILs are shown. TILs were left untreated or treated with increasing concentrations of the pan-AKT inhibitor ipatasertib. Treatment was added either during the pre-REP and REP (blue bars) or during the REP stage only (purple bars). Fold expansion and viability of TIL at the end of the 22-day expansion process are shown. Frequency of CD8⁺, CD4⁺ and CD4⁺ (Foxp3⁺) cells after the expansion process on cryopreserved cells are also shown.

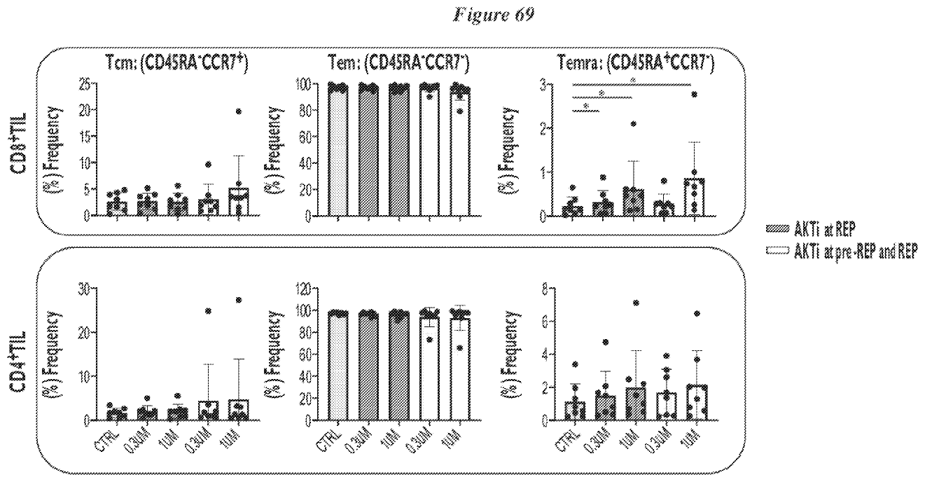

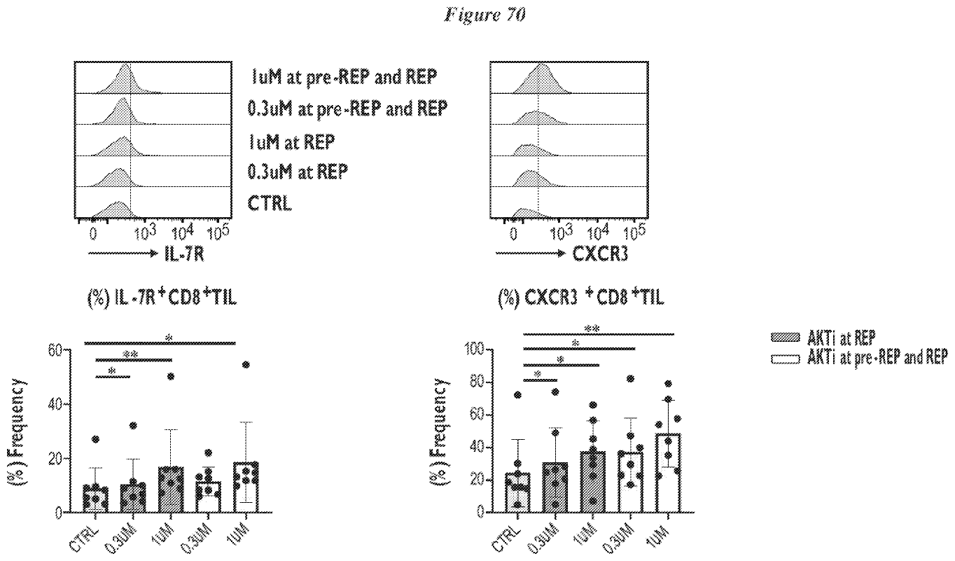

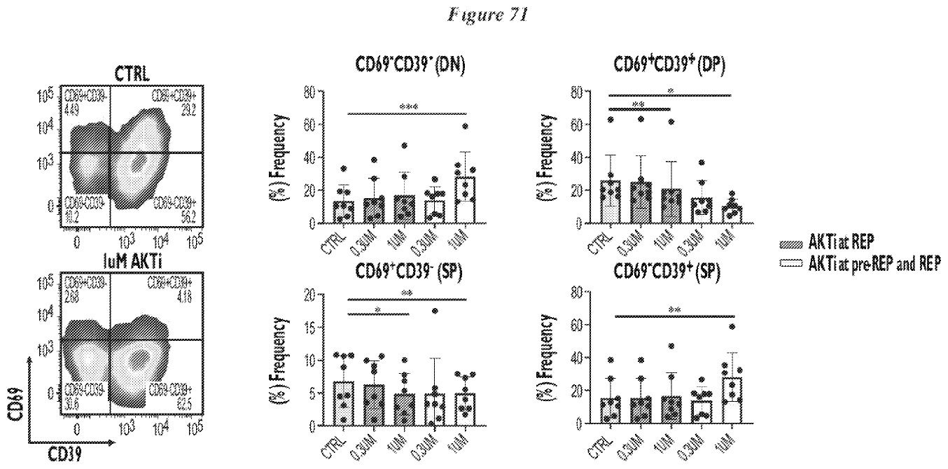

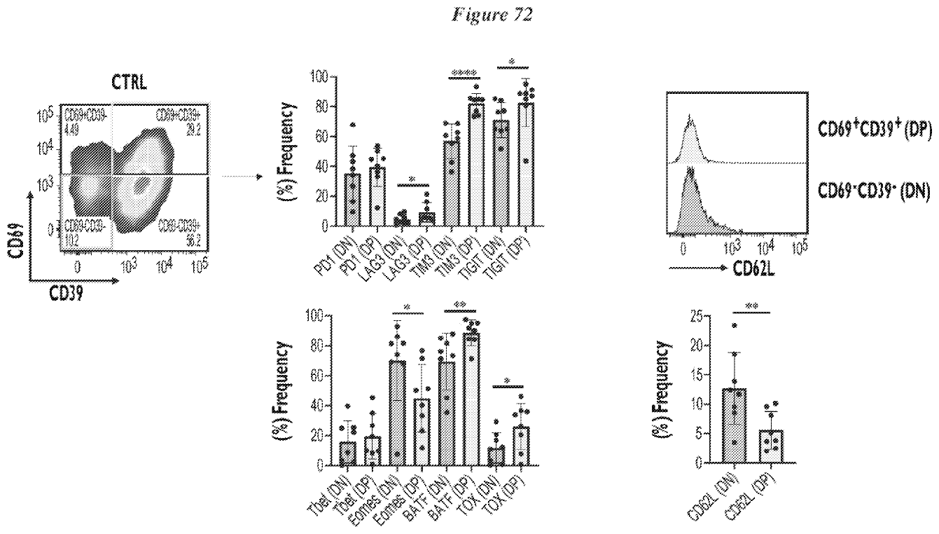

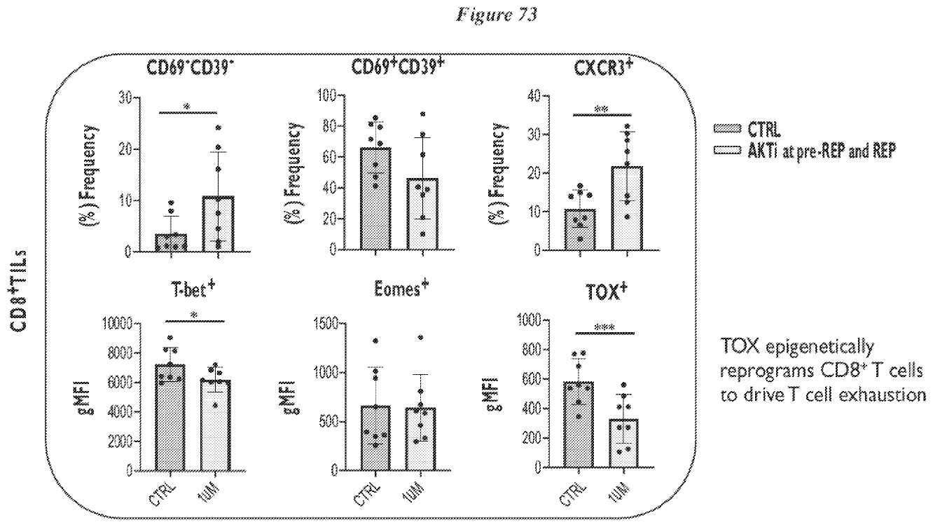

Additional results are shown in FIGS. 69 to 75. AKT inhibition is shown to induce an increase in the frequency of CD8⁺ TEMRA cells in FIG. 69 and an increase in IL-7R and CXCR3 expression on CD8⁺ TILs in FIG. 70. FIG. 71 demonstrates that AKT inhibition increases the frequency of less differentiated CD8⁺CD69⁻CD39⁻ T cells on both CD8⁺ and CD4⁺ TILs. FIG. 72 shows that CD8⁺CD69⁻CD39⁻ TILs are less differentiated and exhausted. AKT inhibitor-treated TILs also maintain a higher frequency of CD8⁺CD69⁻CD39⁻ T cells and lower TOX expression following stimulation, as shown in FIG. 73.

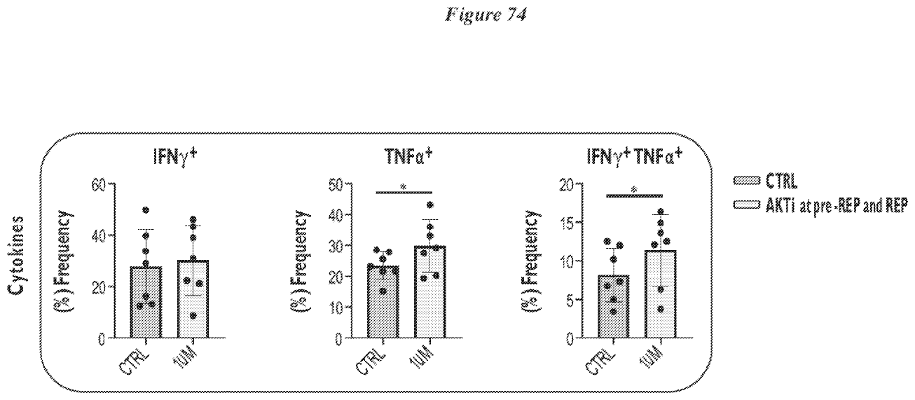

Cryopreserved control and TIL treated at both pre-REP and REP with 1 μM ipatasertib were stimulated overnight with anti-CD3/CD28 beads at a bead-to-cell ratio of 1:5. Results are shown in FIG. 74. AKT inhibitor-treated TILs prepared using the Gen 2 process were observed to maintain a higher cytokine output following stimulation.

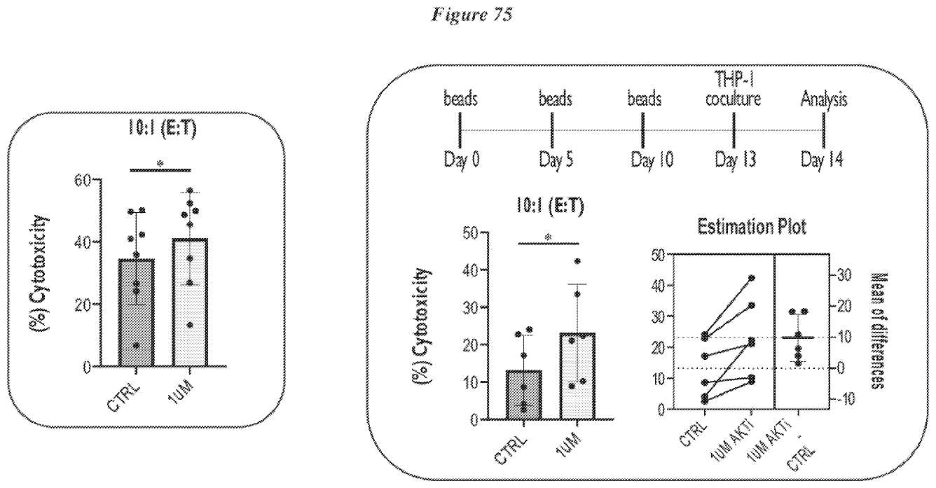

Cryopreserved control and TILs treated at both pre-REP and REP with 1 μM ipatasertib were cocultured for 24 hours with KILR® THP-1 cells at a 10:1 effector-to-target cell ratio to measure cytotoxicity in an allogeneic setting. Control and ipatasertib-treated TILs were stimulated every 5 days with anti-CD3/CD28 beads at a 1:1 bead-to-cell ratio. Three days after the third stimulation, cells were washed, beads were removed, and cells were cocultured at a 10:1 effector-to-target cell ratio with KILR THP-1 cells for 24 hours. As shown in FIG. 75, ipatasertib-treated TILs prepared using the Gen 2 process showed increased cytotoxicity in an allogeneic setting that is sustained after repeated stimulation.

Treatment with an AKT inhibitor at a 1 μM dose led to equivalent expansion and viability of TILs relative to controls but doubled the population of less differentiated CD39⁻ CD69⁻ cells. This effect was present even after re-stimulation and these cells showed reduced expression of CD38 as well as the transcription factors T-bet and TOX, suggesting a less differentiated and exhausted phenotype. Importantly, AKT inhibitor treatment led to an increase in the frequency of IFNγ⁺TNFα⁺CD8⁺ T cells, which translated into increased cytotoxicity. AKT inhibitor treatment during ex vivo TIL expansion augmented the proportion of less differentiated, more memory-like, functional TILs.

Temporally inhibiting AKT signaling during TIL expansion therefore represents an approach for improving the performance of TIL products and augmenting TIL persistence and therapeutic efficacy in the clinical setting, including in combination with CCRs and/or chemokine receptors. AKT inhibitor-treated TILs maintained higher frequencies of CD69⁻CD39⁻ cells with reduced TOX levels and increased cytokine output following stimulation. Increased cytotoxic capacity was observed with AKT inhibitor treated TIL in an allogeneic setting, which was sustained even after repeated TIL stimulation. AKT inhibitor-treated TILs may be further modified to express one or more CCRs or chemokine receptors as described elsewhere herein.

### Example 22: Use of Epigenetic Modifications to Improve Phenotype in TIL Products

In this example, the use of decitabine, a DNA hypomethylation agent, in culture as an epigenetic modifier of TIL products was explored. Decitabine can be combined with AKT inhibitors and the CCRs and chemokine receptors disclosed herein, as well as with other genetically modified TILs described herein. Patient tumors (N=8) from different tumor types (non-small cell lung, head and neck, ovarian, and breast cancers) were obtained from donors, fragmented, and subjected to a 22-day expansion protocol for TIL generation. Different doses (10 nM, 30 nM, and 100 nM) of decitabine were added to the culture during ex vivo expansion either during the pre-REP and REP stages or during REP only. The expansion potential as well as the phenotypic and functional characteristics of TILs were evaluated in the final TIL product.

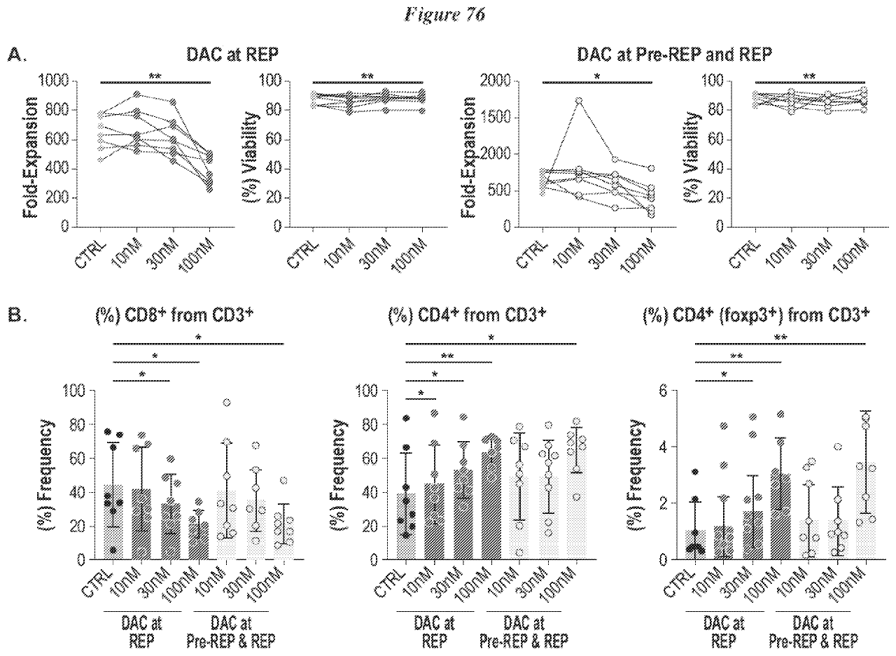

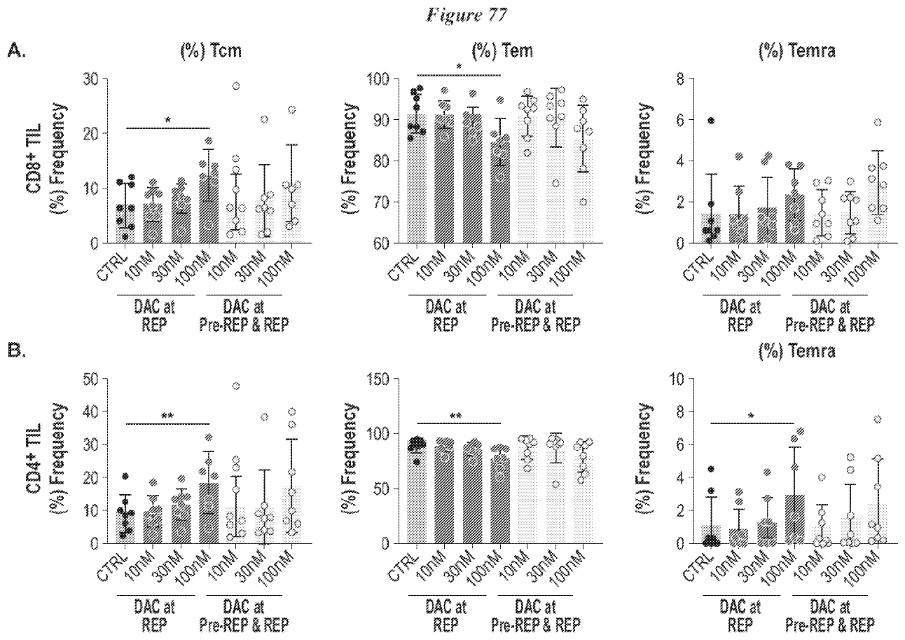

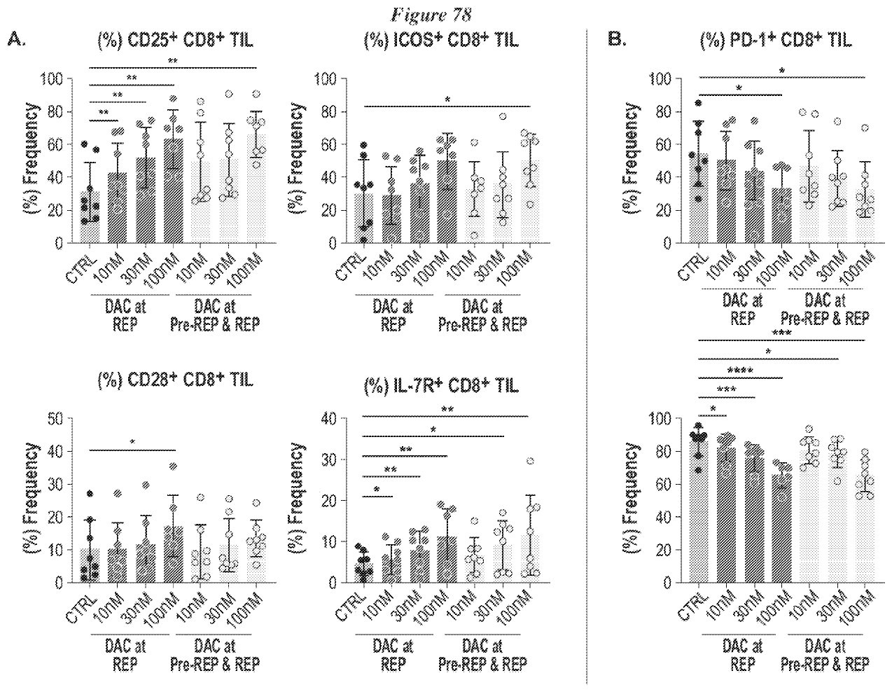

The results shown in FIG. 76 illustrate that decitabine treatment maintained TIL viability, but decreased expansion while increasing the CD4+/CD8+ T-cell ratio. Expansion, viability, and T-cell distribution in control TILs and decitabine-treated TILs are shown in FIG. 76. TILs were left untreated (CTRL, gray bars) or treated with increasing concentrations of decitabine. Treatment was added either during the REP stage only (blue bars) or during both pre-REP and REP (green bars). In FIG. 76, panel A shows fold-expansion and viability of TILs at the end of the 22-day expansion process, while panel B shows the frequency of CD8⁺, CD4⁺, and CD4+(Foxp3+) cells after the expansion process on cryopreserved cells (*P<0.05, **P<0.01). In FIG. 77, results are shown that demonstrate that decitabine treatment during the REP stage increased the frequency of T_{CM}-like cells in both CD8⁺ and CD4⁺ T cells. T-cell subsets in control TILs and decitabine-treated TILs are shown, with the frequency of T_{CM} (CD45RA⁻CCR7⁺), TEM (CD45RA⁻CCR7⁻), and T_{EMRA} (CD45⁺CCR7⁻) cells shown in panel A for CD8⁺ TILs and panel B for CD4⁺ TILs after expansion (*P<0.05, **P<0.01). FIG. 78 shows the expression of surface markers on decitabine-treated TILs. Decitabine treatment is observed to increase the frequency of co-stimulatory receptors while decreasing inhibitory receptor expression on CD8⁺ TILs. Panel A of FIG. 78 shows the expression of CD25, ICOS, CD28, and IL-7R on CD8⁺ TILs, while panel B shows expression of inhibitory receptors PD-1 and TIGIT on CD8⁺ TILs (*P<0.05, **P<0.01, ***P<0.001, ****P<0.0001). Similar results were observed for CD4⁺ TIL.

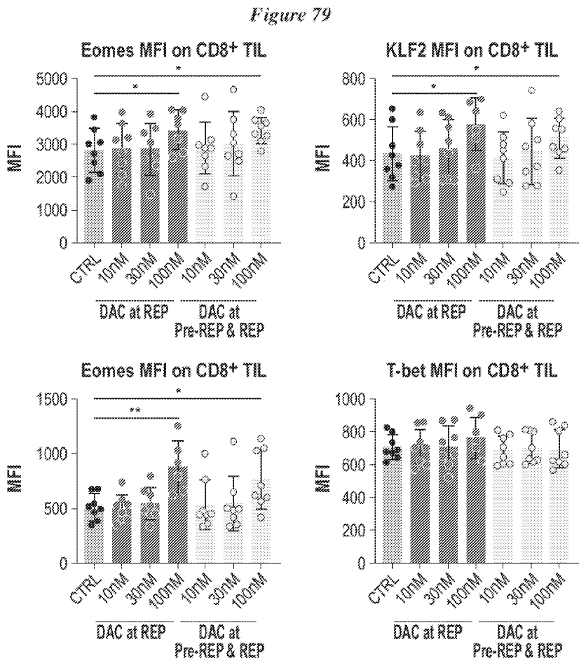

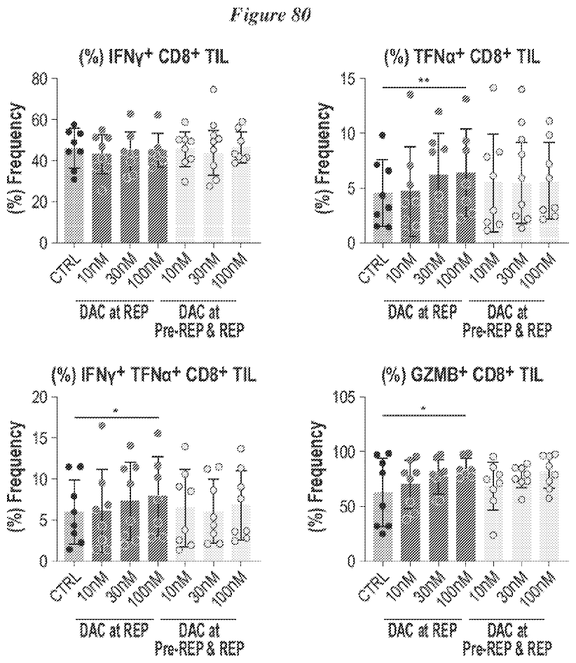

The expression of transcription factors in decitabine-treated TILs is shown in FIG. 79, which demonstrates that decitabine treatment increased the expression of memory-associated transcription factors. Control or decitabine-treated cryopreserved TILs were thawed and stained for flow cytometry analysis. Expression of Eomes, KLF2, BATF, and T-bet on CD8⁺ TILs are shown in FIG. 79 (*P<0.05, **P<0.01). Cytokine expression in control or decitabine-treated TILs following in vitro stimulation is presented in FIG. 80. It was found that decitabine treatment increased the frequency of TNF-α and granzyme B expressing CD8⁺ TILs following stimulation. Cryopreserved control and decitabine-treated TILs were stimulated overnight with anti-CD3/CD28 beads at a bead-to-cell ratio of 1:5. Expression levels of IFN-γ, TNF-α, and granzyme B on CD8⁺ TILs are shown in FIG. 80 (*P<0.05, **P<0.01).

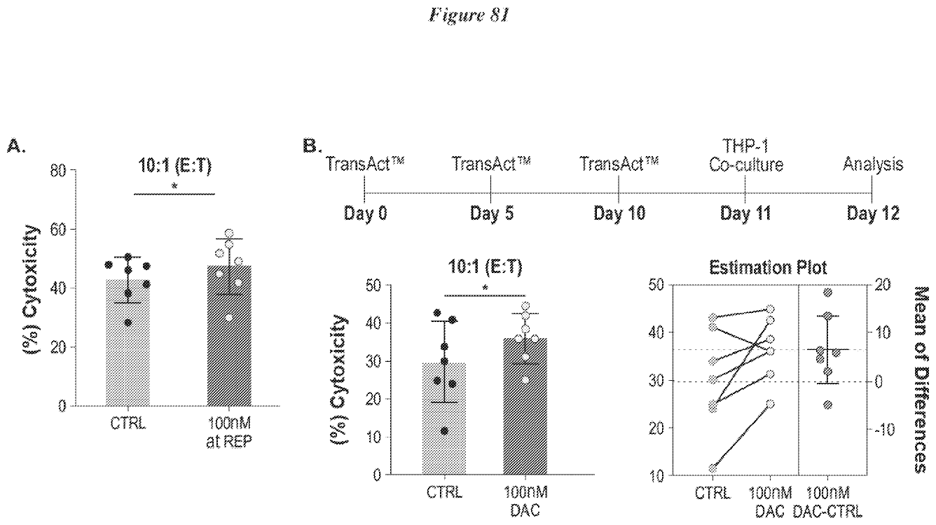

Cytotoxicity of control and decitabine-treated TILs was also assessed. FIG. 81 illustrates that decitabine-treated TIL showed increased cytotoxicity that was sustained after repeated stimulation. In panel A, cryopreserved control and TILs treated at REP with 100 nM decitabine were cocultured for 24 hours with KILR® THP-1 cells (Eurofins DiscoverX, Fremont, CA, USA) at a 10:1 E:T cell ratio to measure cytotoxicity in an allogeneic setting. In panel B, control TILs and decitabine-treated TILs were stimulated every 5 days with TransActtm (Miltenyi Biotec, Germany). One day after the third stimulation, cells were washed and cocultured at a 10:1 effector-to-target cell ratio with KILR THP-1 cells for 24 h to measure cytotoxicity. *P<0.05.

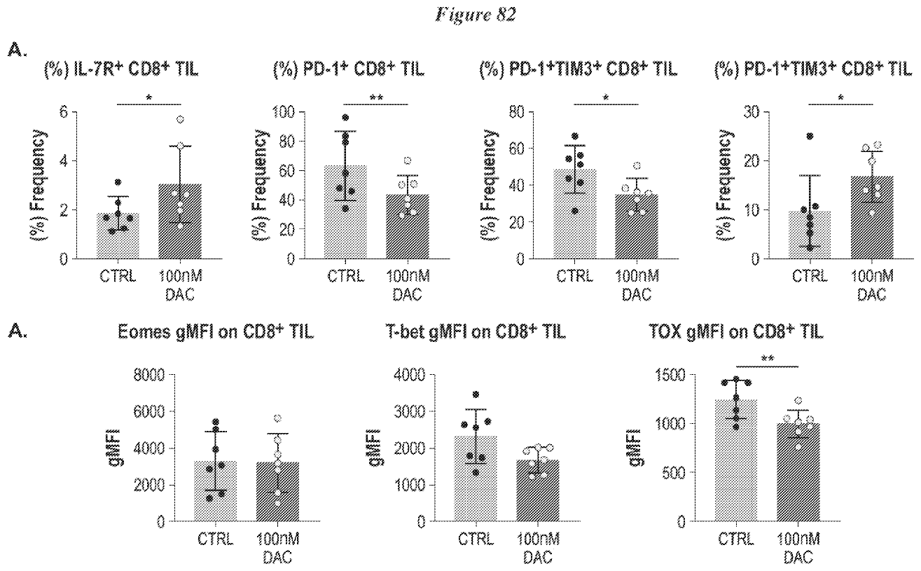

FIG. 82 illustrates that decitabine-treated TILs showed reduced inhibitory receptor expression and lower levels of TOX while having increased IL-7R expression after repeated stimulation. The phenotype of control- and decitabine-treated TILs are shown after repeated stimulation. Control TILs and decitabine-treated TILs were stimulated every 5 days with TransActtm (Miltenyi Biotec, Germany). One day after the third stimulation, cells were washed and stained for flow cytometry analysis. In FIG. 82, expression of IL-7R, PD-1, and TIM3 in TIL after repeated stimulation is shown in panel A, while expression levels of transcription factors in TIL after repeated stimulation are shown in panel B (*P<0.05, **P<0.01).

In conclusion, decitabine treatment during TIL expansion can shift the balance away from effector differentiation and toward a more memory-like phenotype. Decitabine treatment at 100 nM during only the REP stage increased the expression of costimulatory receptors while reducing inhibitory receptor expression. Decitabine treatment increased the frequency of TNFα⁺ and IFNγ⁺TNFα⁺CD8⁺ TILs while conferring increased killing activity, which was sustained even after repeated stimulation. Decitabine-treated TILs showed reduced TOX levels and lower frequency of PD1⁺ TIM3⁺CD8⁺ TILs following repeated stimulation. This provides evidence that inhibiting DNA methylation programs during TIL expansion modifying the epigenetic landscape of TILs to improve their therapeutic potential.

### Example 23: Biepitope TROP-2 and PD-L1 Chimeric Costimulatory Receptors

In this example, additional biepitope CCRs targeting TROP-2 and PD-L1 are prepared and tested, again using PD-L1 scFvs based on 38A1 and 19H-9 and TROP-2 scFvs based on cAR47A6.4 and KM4097, using procedures as described above. These CCRs are designated CCR7.2, CCR8.2, CCR11.2, and CCR12.2. Suitable, non-limiting embodiments of CCRs prepared according to this example and useful as CCR constructs of the present invention are set forth in Table 87.

TABLE 87
Amino acid sequences of exemplary biepitope CCRs designated CCR7.2,
CCR8.2, CCR11.2, and CCR12.2.

| Identifier | Sequence (One-Letter Amino Acid Symbols) | |
|---|---|---|
| SEQ ID NO: 677 | MALPVTALLL PLALLLHAAR PSYVLTQPPS VSVAPGQTAR ITCGGNNIGR KIVHWYQQRP | 60 |
| CCR7.2: chPD-L1- | GQAPVLVIYY DTDRPAGIPE RFSGSNSGNM ATLTISTVGA GDEADYYCQV WDTGSDHVVF | 120 |
| IL-2R | GGGTKLTVLG GGGSGGGGSG GGGSGGGGSE VQLVESGGGL VQPGGSLRLS CAASGFTFSN | 180 |
| | YAMSWVRQAP GKGLEWVSTI SGSGGTTYYA DSVKGRFTIS RDNSKNTLYL QMNSLRVEDT | 240 |
| | AVYYCAKDWF RSSSPDAFDI WGQGTTVTVS AFRTKPAALG KDTIPWLGHL LVGLSGAFGF | 300 |
| | IILVYLLINC RNTGPWLKKV LKCNTPDPSK FFSQLSSEHG GDVQKWLSSP FPSSSFSPGG | 360 |
| | LAPEISPLEV LERDKVTQLL LQQDKVPEPA SLSSNHSLTS CFTNQGYFFF HLPDALEIEA | 420 |
| | CQVYFTYDPY SEEDPDEGVA GAPTGSSPQP LQPLSGEDDA YCTFPSRDDL LLFSPSLLGG | 480 |
| | PSPPSTAPGG SGAGEERMPP SLQERVPRDW DPQPLGPPTP GVPDLVDFQP PPELVLREAG | 540 |
| | EEVPDAGPRE GVSFPWSRPP GQGEFRALNA RLPLNTDAYL SLQELQGQDP THLVGSGEGR | 600 |
| | GSLLTCGDVE ENPGPMALPV TALLLPLALL LHAARPNFML TQPHSVSESL GKTVTISCTG | 660 |
| | SSGSIARKFV QWYQQRPGSS PTTVIYENNQ RPSGVSDRFS GSIGSSSNSA SLTISGLKTE | 720 |
| | DEADYYCQSY DSSNVVFGGG TKVTVLGGGG SGGGGSGGGG SGGGGSQVQL QESGGGLVKP | 780 |
| | GGSIRLSCAA SGFTFSSYSM NWVRQAPGKG LEWVSGINTA GDTHYPESVK GRFTISRDNA | 840 |
| | RNSINLQMNS LRAEDTAVYY CVRERVEREY SGYDAFDIWG QGTTVTVSAS KENPFLFALE | 900 |
| | AVVISVGSMG LIISLLCVYF WLERTMPRIP TIKNLEDLVT EYHGNFSAWS GVSKGLAESL | 960 |
| | QPDYSERLCL VSEIPPKGGA LGEGPGASPC NQHSPYWAPP CYTLKPET | 1008 |
| SEQ ID NO: 678 | MALPVTALLL PLALLLHAAR PSYVLTQPPS VSVAPGQTAR ITCGGNNIGR KIVHWYQQRP | 60 |
| CCR8.2: chPD-L1- | GQAPVLVIYY DTDRPAGIPE RFSGSNSGNM ATLTISTVGA GDEADYYCQV WDTGSDHVVF | 120 |
| IL-18R | GGGTKLTVLG GGGSGGGGSG GGGSGGGGSE VQLVESGGGL VQPGGSLRLS CAASGFTFSN | 180 |
| | YAMSWVRQAP GKGLEWVSTI SGSGGTTYYA DSVKGRFTIS RDNSKNTLYL QMNSLRVEDT | 240 |
| | AVYYCAKDWF RSSSPDAFDI WGQGTTVTVS ADMADIPGHV FTRGMIIAVL ILVAVVCLVT | 300 |
| | VCVIYRVDLV LFYRHLTRRD ETLTDGKTYD AFVSYLKECR PENGEEHTFA VEILPRVLEK | 360 |
| | HFGYKLCIFE RDVVPGGAVV DEIHSLIEKS RRLIIVLSKS YMSNEVRYEL ESGLHEALVE | 420 |
| | RKIKIILIEF TPVTDFTFLP QSLKLLKSHR VLKWKADKSL SYNSRFWKNL LYLMPAKTVK | 480 |
| | PGRDEPEVLP VLSESGSGEG RGSLLTCGDV EENPGPMALP VTALLLPLAL LLHAARPNEM | 540 |
| | LTQPHSVSES LGKTVTISCT GSSGSIARKF VQWYQQRPGS SPTTVIYENN QRPSGVSDRF | 600 |
| | SGSIGSSSNS ASLTISGLKT EDEADYYCQS YDSSNVVEGG GTKVTVLGGG GSGGGGSGGG | 660 |
| | GSGGGGSQVQ LQESGGGLVK PGGSLRLSCA ASGFTFSSYS MNWVRQAPGK GLEWVSGINT | 720 |
| | AGDTHYPESV KGRFTISRDN ARNSLNLQMN SIRAEDTAVY YCVRERVERE YSGYDAFDIW | 780 |
| | GQGTTVTVSA NTTQSVQLKE KRGVVLLYIL LGTIGTLVAV LAASALLYRH WIEIVLLYRT | 840 |
| | YQSKDQTLGD KKDFDAFVSY AKWSSFPSEA TSSLSEEHLA LSLFPDVLEN KYGYSLCLLE | 900 |
| | RDVAPGGVYA EDIVSIIKRS RRGIFILSPN YVNGPSIFEL QAAVNLALDD QTLKLILIKF | 960 |
| | CYFQEPESLP HLVKKALRVL PTVTWRGLKS VPPNSRFWAK MRYHMPVKNS QGFTWNQLRI | 1020 |
| | TSRIFQWKGL SRTETTGRSS QPKEW | 1045 |
| SEQ ID NO: 679 | MALPVTALLL PLALLLHAAR PQIQLVQSGP ELKKPGETVK ISCKASGYTF TNYGMNWVKQ | 60 |
| CCR11.2: TROP2- | APGKGLKWMG WINTKTGEPT YAEEFKGRFA FSLETSASTA YLQINNLKKE DTATYFCGRG | 120 |
| IL-2R | GYGSSYWYFD VWGAGTTVTV SSASTKGPSG GGGSGGGGSG GGGSGGGGSD IVMTQSHKFM | 180 |
| | STSVGDRVSI TCKASQDVSI AVAWYQQKPG QSPKVLIYSA SYRYTGVPDR FTGSGSGTDF | 240 |
| | TFTISRVQAE DLAVYYCQQH YITPLTFGAG TKLELKRTVA FRTKPAALGK DTIPWLGHLL | 300 |
| | VGLSGAFGFI ILVYLLINCR NTGPWLKKVL KCNTPDPSKF FSQLSSEHGG DVQKWLSSPF | 360 |
| | PSSSFSPGGL APEISPLEVL ERDKVTQLLL QQDKVPEPAS LSSNHSLTSC FTNQGYFFFH | 420 |
| | LPDALEIEAC QVYFTYDPYS EEDPDEGVAG APTGSSPQPL QPLSGEDDAY CTFPSRDDLL | 480 |
| | LFSPSLLGGP SPPSTAPGGS GAGEERMPPS LQERVPRDWD PQPLGPPTPG VPDLVDFQPP | 540 |
| | PELVLREAGE EVPDAGPREG VSFPWSRPPG QGEFRALNAR LPINTDAYLS LQELQGQDPT | 600 |
| | HLVGSGEGRG SLLTCGDVEE NPGPMALPVT ALLLPLALLL HAARPQVQLQ QSGPELVRPG | 660 |
| | TSVRISCKAS GYTFTIYWLG WVKQRPGHGL EWIGNIFPGS AYINYNEKFK GKATLTADTS | 720 |
| | SSTAYMQLSS LTSEDSAVYF CAREGSNSGY WGQGTTLTVS SGGGGSGGGG SGGGGSGGGG | 780 |
| | SDIVMTQSPS SLSVSAGEKV TMTCKSSQSL LNSGNQQNYL AWYQQKPGQP PKLLIYGAST | 840 |
| | RESGVPDRFT GSGSGTDFTL TINSVQAEDL AVYYCQSDHI YPYTFGGGTK LEIKSKENPF | 900 |
| | LFALEAVVIS VGSMGLIISL LCVYFWLERT MPRIPTLKNL EDLVTEYHGN FSAWSGVSKG | 960 |
| | LAESLQPDYS ERLCLVSEIP PKGGALGEGP GASPCNQHSP YWAPPCYTLK PET | 1013 |
| SEQ ID NO: 680 | MALPVTALLL PLALLLHAAR PQIQLVQSGP ELKKPGETVK ISCKASGYTF TNYGMNWVKQ | 60 |
| CCR12.2: TROP2- | APGKGLKWMG WINTKTGEPT YAEEFKGRFA FSLETSASTA YLQINNLKKE DTATYFCGRG | 120 |
| IL-18R | GYGSSYWYFD VWGAGTTVTV SSASTKGPSG GGGSGGGGSG GGGSGGGGSD IVMTQSHKFM | 180 |
| | STSVGDRVSI TCKASQDVSI AVAWYQQKPG QSPKVLIYSA SYRYTGVPDR FTGSGSGTDF | 240 |
| | TFTISRVQAE DLAVYYCQQH YITPLTFGAG TKLELKRTVA DMADIPGHVF TRGMIIAVLI | 300 |
| | LVAVVCLVTV CVIYRVDLVL FYRHLTRRDE TLTDGKTYDA FVSYLKECRP ENGEEHTFAV | 360 |
| | EILPRVLEKH FGYKLCIFER DVVPGGAVVD EIHSLIEKSR RLIIVLSKSY MSNEVRYELE | 420 |
| | SGLHEALVER KIKIILIEFT PVTDFTFLPQ SLKLLKSHRV LKWKADKSLS YNSRFWKNLL | 480 |
| | YLMPAKTVKP GRDEPEVLPV LSESGSGEGR GSLLTCGDVE ENPGPMALPV TALLLPLALL | 540 |
| | LHAARPQVQL QQSGPELVRP GTSVRISCKA SGYTFTIYWL GWVKQRPGHG LEWIGNIFPG | 600 |
| | SAYINYNEKF KGKATLTADT SSSTAYMQLS SITSEDSAVY FCAREGSNSG YWGQGTTLTV | 660 |
| | SSGGGGSGGG GSGGGGSGGG GSDIVMTQSP SSLSVSAGEK VTMTCKSSQS LLNSGNQQNY | 720 |
| | LAWYQQKPGQ PPKLLIYGAS TRESGVPDRF TGSGSGTDFT LTINSVQAED LAVYYCQSDH | 780 |
| | IYPYTFGGGT KLEIKNTTQS VQLKEKRGVV LLYILLGTIG TLVAVLAASA LLYRHWIEIV | 840 |
| | LLYRTYQSKD QTLGDKKDFD AFVSYAKWSS FPSEATSSLS EEHLALSLFP DVLENKYGYS | 900 |
| | LCLLERDVAP GGVYAEDIVS IIKRSRRGIF ILSPNYVNGP SIFELQAAVN LALDDQTLKL | 960 |
| | ILIKFCYFQE PESLPHLVKK ALRVLPTVTW RGLKSVPPNS RFWAKMRYHM PVKNSQGFTW | 1020 |
| | NQLRITSRIF QWKGLSRTET TGRSSQPKEW | 1050 |

Suitable, non-limiting embodiments of nucleotides encoding the CCRs prepared according to this example and useful as CCR constructs of the present invention are set forth in Table 88.

TABLE 88
Nucleotide sequences of exemplary biepitope CCRs designated CCR7.2,
CCR8.2, CCR11.2, and CCR12.2.

| Identifier | Sequence (One-Letter Nucleotide Symbols) | |
|---|---|---|
| SEQ ID NO: 681 | ATGGCCTTGC CCGTCACTGC CTTGCTGCTG CCTCTGGCCT TACTGCTGCA CGCCGCCAGA | 60 |
| CCR7.2: chPD-L1- | CCTAGCTACG TGCTGACCCA GCCTCCTAGC GTGAGCGTGG CCCCTGGCCA GACCGCTAGA | 120 |
| IL-2R | ATCACCTGCG GCGGCAACAA CATCGGAAGA AAGATCGTGC ACTGGTATCA GCAGCGGCCT | 180 |
| | GGCCAAGCTC CTGTGCTGGT GATTTACTAC GACACCGACA GACCAGCCGG TATCCCCGAG | 240 |
| | CGGTTCAGCG GCTCTAACAG CGGAAATATG GCGACACTGA CCATCTCCAC CGTGGGCGCC | 300 |
| | GGCGATGAGG CCGACTACTA CTGCCAAGTG TGGGATACAG GCTCTGACCA CGTGGTGTTC | 360 |
| | GGCGGGGGCA CCAAGCTGAC CGTGCTGGGA GGCGGCGGCA GCGGCGGCGG CGGCAGCGGC | 420 |
| | GGCGGCGGCA GCGGCGGAGG AGGAAGCGAG GTGCAACTGG TCGAATCCGG CGGAGGGCTG | 480 |
| | GTACAGCCTG GTGGCTCCTT GCGGCTGAGC TGCGCCGCTT CTGGCTTCAC CTTCAGCAAT | 540 |
| | TACGCCATGA GCTGGGTTAG ACAGGCCCCT GGGAAGGGCC TGGAATGGGT GAGCACCATC | 600 |
| | TCAGGCAGCG GAGGCACAAC CTACTACGCC GATAGCGTGA AAGGCAGGTT CACAATCAGC | 660 |
| | AGAGATAATA GCAAAAACAC CCTGTACCTG CAAATGAACA GCCTGAGAGT GGAAGATACA | 720 |
| | GCCGTGTATT ACTGTGCCAA GGACTGGTTC AGAAGCTCCA GCCCTGACGC CTTTGACATT | 780 |
| | TGGGGCCAGG GGACCACCGT GACCGTGAGT GCTTTCCGGA CCAAGCCTGC CGCCCTGGGA | 840 |
| | AAAGACACCA TCCCTTGGCT GGGCCACCTG CTAGTGGGCC TTTCGGGAGC CTTCGGCTTC | 900 |
| | ATCATCCTGG TGTACCTGCT GATCAACTGC AGAAACACCG GCCCTTGGCT GAAAAAGGTG | 960 |
| | CTCAAGTGCA ACACACCTGA CCCTAGCAAG TTCTTCTCTC AGCTGAGCAG CGAGCACGGA | 1020 |
| | GGCGATGTGC AGAAATGGCT GAGCAGCCCC TTCCCTAGCA GCTCTTTCAG CCCTGGAGGC | 1080 |
| | CTGGCCCCTG AAATCTCTCC TCTGGAAGTG CTGGAGAGAG ACAAGGTGAC CCAGCTGCTG | 1140 |
| | CTGCAGCAAG ACAAGGTGCC CGAACCCGCC AGCCTGAGCA GCAACCACAG CCTGACCAGC | 1200 |
| | TGTTTTACAA ATCAGGGCTA CTTCTTCTTC CACCTGCCTG ATGCCCTGGA GATCGAGGCC | 1260 |
| | TGTCAGGTGT ACTTCACCTA CGATCCCTAC TCCGAGGAGG ACCCTGACGA GGGCGTGGCC | 1320 |
| | GGTGCTCCAA CAGGCAGCTC GCCTCAGCCT CTGCAGCCTC TCAGCGGCGA GGACGACGCT | 1380 |
| | TATTGCACCT TTCCTAGCAG AGACGACCTG CTGCTTTTCA GCCCCTCCCT GCTCGGCGGC | 1440 |
| | CCCTCCCCCC CCAGCACCGC CCCAGGCGGA AGCGGCGCCG GCGAGGAGCG GATGCCTCCT | 1500 |
| | TCTCTGCAGG AGAGAGTGCC TCGGGATTGG GACCCTCAGC CTCTGGGACC TCCTACCCCT | 1560 |
| | GGCGTGCCCG ACCTGGTGGA CTTCCAGCCC CCTCCCGAGC TGGTCCTGAG AGAAGCCGGC | 1620 |
| | GAGGAAGTGC CCGACGCCGG ACCTCGGGAA GGCGTGTCCT TCCCCTGGTC CCGGCCTCCA | 1680 |
| | GGCCAGGGCG AGTTCAGAGC GCTGAATGCC AGACTGCCAC TGAACACCGA CGCCTACCTG | 1740 |
| | AGCCTGCAGG AGCTGCAGGG CCAGGACCCG ACCCACCTGG TGGGCTCTGG CGAAGGCCGG | 1800 |
| | GGCAGCCTGC TGACCTGTGG CGATGTGGAA GAGAACCCTG GCCCTATGGC TCTCCCTGTT | 1860 |
| | ACAGCCCTGC TTCTGCCTCT GGCTCTCCTG CTGCATGCTG CGCGGCCCAA CTTTATGCTG | 1920 |
| | ACCCAGCCCC ACAGCGTCAG TGAAAGCCTG GGCAAAACCG TGACAATCAG CTGCACAGGC | 1980 |
| | AGCTCAGGAA GCATCGCCAG GAAGTTCGTG CAGTGGTATC AGCAACGGCC AGGATCATCT | 2040 |
| | CCTACAACCG TGATCTACGA GAACAACCAG AGACCTTCTG GCGTGAGTGA TAGATTCAGC | 2100 |
| | GGCTCTATCG GCAGTAGCTC TAATAGCGCC TCTCTGACAA TCAGCGGCCT GAAGACCGAG | 2160 |
| | GATGAGGCCG ATTACTATTG CCAGTCCTAC GACAGCAGCA ACGTGGTGTT CGGCGGCGGC | 2220 |
| | ACAAAGGTTA CCGTCCTGGG CGGCGGAGGC TCTGGCGGCG GCGGCAGCGG CGGCGGAGGC | 2280 |
| | TCTGGCGGCG GCGGTAGCCA GGTGCAACTG CAAGAATCCG GAGGCGGCCT GGTCAAGCCT | 2340 |
| | GGTGGGAGCC TGAGACTGAG CTGCGCCGCC AGCGGCTTCA CATTCTCCAG CTACAGCATG | 2400 |
| | AACTGGGTCC GGCAGGCCCC AGGCAAGGGC CTGGAGTGGG TCAGCGGAAT CAACACCGCC | 2460 |
| | GGCGACACAC ATTACCCTGA GAGCGTGAAG GGCAGATTTA CCATCAGCAG AGACAACGCC | 2520 |
| | CGGAACAGCC TGAACCTGCA GATGAATAGC CTGCGGGCCG AAGATACAGC CGTTTACTAC | 2580 |
| | TGCGTGAGAG AGAGAGTGGA ACGCGAGTAC AGCGGATATG ACGCCTTCGA CATCTGGGGC | 2640 |
| | CAGGGAACGA CCGTGACAGT GTCGGCCAGC AAGGAAAATC CTTTTCTGTT CGCCCTGGAG | 2700 |
| | GCCGTCGTAA TCAGCGTGGG AAGCATGGGC CTGATCATCA GCCTGCTGTG TGTGTACTTT | 2760 |
| | TGGCTGGAGC GTACCATGCC TAGAATCCCC ACCCTGAAGA ACCTGGAGGA CCTGGTGACA | 2820 |
| | GAATACCACG GCAACTTCAG CGCTTGGAGC GGTGTGTCTA AGGGACTGGC CGAGTCTCTG | 2880 |
| | CAGCCAGACT ACAGCGAGCG GCTGTGCCTG GTGAGCGAAA TCCCACCAAA GGGAGGGGCT | 2940 |
| | CTCGGCGAAG GCCCTGGCGC TTCCCCTTGT AACCAGCACT CTCCCTACTG GGCTCCTCCA | 3000 |
| | TGCTACACCC TGAAGCCTGA AACC | 3024 |
| SEQ ID NO: 682 | ATGGCCCTGC CAGTGACAGC CCTGCTTCTG CCTCTGGCTC TGCTGCTCCA CGCCGCCAGA | 60 |
| CCR8.2: chPD-L1- | CCCAGCTACG TGCTCACACA GCCTCCTTCT GTGAGCGTGG CCCCAGGACA GACAGCCAGA | 120 |
| IL-18R | ATCACCTGTG GCGGCAACAA CATCGGCAGA AAGATCGTGC ACTGGTATCA GCAAAGACCT | 180 |
| | GGCCAGGCCC CTGTGCTGGT GATCTACTAC GATACTGATC GGCCCGCCGG CATCCCTGAG | 240 |
| | CGGTTTTCTG GCTCTAACTC CGGAAATATG GCAACACTGA CAATCAGCAC AGTGGGCGCC | 300 |
| | GGAGACGAAG CTGACTACTA CTGCCAGGTG TGGGATACTG GCTCGGACCA CGTGGTTTTC | 360 |
| | GGCGGCGGCA CCAAGCTGAC CGTCCTGGGC GGCGGTGGTT CGGGCGGCGG CGGCTCTGGC | 420 |
| | GGCGGCGGCT CTGGCGGAGG CGGCAGCGAG GTGCAGCTGG TTGAAAGCGG CGGCGGTCTG | 480 |
| | GTGCAGCCTG GAGGCTCCTT ACGGCTGTCC TGCGCCGCCA GCGGCTTCAC TTTCAGCAAC | 540 |
| | TACGCCATGA GCTGGGTCCG GCAGGCCCCT GGAAAAGGCC TGGAATGGGT GAGCACCATC | 600 |
| | AGCGGAAGCG GCGGGACCAC CTATTACGCC GACAGCGTGA AGGGCAGATT TACCATCAGC | 660 |
| | AGGGACAATA GCAAGAATAC CCTGTACCTG CAGATGAACA GCCTGCGAGT GGAGGACACA | 720 |
| | GCCGTCTACT ACTGCGCCAA GGACTGGTTC AGAAGCAGCT CTCCTGACGC CTTCGACATC | 780 |
| | TGGGGCCAGG GCACCACCGT GACTGTCAGC GCCGACATGG CCGACATCCC TGGCCACGTG | 840 |
| | TTTACCAGAG GCATGATCAT CGCCGTGCTG ATTCTGGTGG CCGTGGTTTG TCTGGTGACC | 900 |
| | GTGTGCGTGA TCTACCGGGT GGACCTGGTG CTGTTCTACC GGCATCTGAC AAGAAGGGAC | 960 |
| | GAAACCTTGA CGGACGGCAA GACATACGAC GCATTCGTGT CTTACCTGAA GGAGTGCAGA | 1020 |
| | CCCGAGAACG GCGAAGAACA CACCTTTGCC GTGGAAATCC TGCCTAGAGT GCTGGAAAAG | 1080 |
| | CACTTCGGCT ACAAGCTGTG CATCTTCGAG AGAGATGTTG TGCCCGGCGG AGCTGTCGTG | 1140 |
| | GATGAGATAC ACAGTCTGAT CGAGAAAAGC AGAAGACTGA TTATCGTGCT CTCCAAGAGC | 1200 |
| | TATATGAGCA ACGAGGTGAG ATACGAGCTG GAGAGCGGTC TCCACGAGGC TCTGGTCGAA | 1260 |
| | CGGAAGATCA AGATTATTCT GATCGAGTTC ACCCCTGTTA CAGACTTCAC CTTCCTGCCT | 1320 |
| | CAGAGCCTGA AGTTACTGAA GAGTCACAGA GTGCTGAAGT GGAAAGCTGA TAAGAGCCTG | 1380 |
| | AGCTACAACA GCCGCTTCTG GAAGAATCTG CTCTACCTGA TGCCTGCCAA AACAGTGAAG | 1440 |
| | CCTGGCAGGG ATGAGCCTGA GGTGCTGCCC GTGCTGTCTG AAAGCGGCTC CGGGGAGGGC | 1500 |
| | AGAGGCTCCC TGCTGACCTG CGGCGACGTG GAAGAGAACC CCGGACCTAT GGCTCTGCCA | 1560 |
| | GTGACCGCTC TGCTGCTGCC TCTGGCCCTG CTGCTGCATG CCGCCAGGCC TAACTTCATG | 1620 |
| | CTGACCCAAC CTCACAGCGT GTCGGAGTCT CTGGGCAAGA CCGTGACAAT CAGCTGCACC | 1680 |
| | GGAAGCTCTG GCAGCATCGC CCGTAAATTC GTGCAGTGGT ATCAGCAGAG GCCGGGCTCA | 1740 |
| | TCCCCTACCA CCGTGATCTA CGAGAACAAC CAGCGGCCTA GCGGCGTGAG CGACAGATTC | 1800 |
| | AGCGGAAGCA TCGGAAGCAG CAGCAATAGC GCCTCACTGA CCATCAGCGG CCTGAAAACC | 1860 |
| | GAGGACGAAG CCGACTACTA CTGTCAGAGC TACGACTCCA GCAACGTGGT TTTCGGCGGC | 1920 |
| | GGAACAAAGG TGACCGTGCT GGGCGGCGGC GGATCTGGTG GCGGCGGCTC CGGCGGAGGC | 1980 |
| | GGCTCTGGGG GCGGCGGTAG CCAAGTGCAG CTGCAGGAGA GCGGAGGCGG CCTGGTGAAG | 2040 |
| | CCAGGCGGCT CCCTGAGACT GAGCTGTGCC GCTAGTGGCT TTACCTTTAG CAGTTACAGC | 2100 |
| | ATGAACTGGG TGAGACAGGC CCCCGGCAAG GGACTGGAAT GGGTGTCAGG CATCAACACC | 2160 |
| | GCTGGCGATA CACACTACCC CGAGAGCGTG AAAGGCAGAT TCACAATCAG CAGAGATAAC | 2220 |
| | GCCAGAAACA GCCTGAACCT GCAGATGAAT AGCTTGCGGG CCGAGGACAC CGCCGTGTAC | 2280 |
| | TACTGCGTGC GGGAACGGGT GGAAAGAGAG TACTCGGGAT ACGACGCCTT CGACATCTGG | 2340 |
| | GGACAGGGAA CCACAGTGAC AGTGTCCGCC AACACGACCC AGAGCGTACA ACTCAAAGAA | 2400 |
| | AAGCGGGGAG TGGTCCTGCT GTATATCCTG CTCGGCACTA TCGGCACCCT GGTGGCCGTC | 2460 |
| | CTGGCCGCCA GCGCCCTGCT GTATAGACAC TGGATTGAGA TCGTGCTCCT GTACAGAACC | 2520 |
| | TACCAGAGCA AAGACCAGAC GCTGGGCGAC AAGAAGGATT TCGACGCCTT TGTGAGCTAC | 2580 |
| | GCCAAATGGT CCTCCTTCCC TAGCGAGGCC ACATCTAGTC TGTCTGAGGA ACACCTGGCC | 2640 |
| | CTGTCCCTTT TCCCCGATGT GCTGGAGAAC AAGTACGGCT ACAGCCTGTG CCTGCTGGAA | 2700 |
| | CGGGATGTGG CTCCTGGAGG CGTGTACGCC GAAGACATCG TGTCTATCAT CAAAAGAAGC | 2760 |
| | CGGAGAGGCA TCTTCATCCT GTCTCCAAAC TACGTGAACG GCCCTAGCAT CTTCGAGCTG | 2820 |
| | CAAGCGGCTG TTAACTTGGC TCTGGACGAC CAGACCCTGA AGCTGATCCT GATCAAGTTC | 2880 |
| | TGCTACTTCC AGGAGCCTGA GTCCCTGCCC CACCTGGTGA AGAAGGCCCT CAGAGTGCTC | 2940 |
| | CCAACCGTTA CATGGGGGG CCTGAAGAGC GTGCCCCCCA ACTCCAGATT TTGGGCCAAG | 3000 |
| | ATGAGATACC ACATGCCTGT GAAGAATAGC CAAGGCTTCA CCTGGAACCA GCTGCGGATC | 3060 |
| | ACCAGCCGTA TCTTCCAGTG GAAGGGCCTG TCTAGGACCG AGACAACCGG CAGATCTAGC | 3120 |
| | CAGCCTAAGG AATGG | 3135 |
| SEQ ID NO: 683 | ATGGCCCTGC CTGTGACAGC GCTGCTGCTG CCACTGGCCC TGCTTCTGCA CGCCGCCAGA | 60 |
| CCR11.2: TROP2- | CCCCAGATCC AGCTGGTGCA GTCAGGCCCT GAACTGAAGA AGCCTGGAGA GACAGTGAAG | 120 |
| IL-2R | ATTAGCTGCA AGGCCTCGGG ATACACCTTC ACAAACTATG GCATGAACTG GGTCAAACAG | 180 |
| | GCCCCTGGCA AGGGCCTTAA ATGGATGGGC TGGATCAACA CAAAGACCGG CGAACCCACC | 240 |
| | TACGCCGAGG AGTTCAAGGG CAGATTCGCC TTTTCTCTGG AGACATCCGC CAGCACCGCC | 300 |
| | TACCTGCAGA TCAACAACCT GAAAAAGGAA GATACAGCCA CATACTTCTG CGGACGGGGC | 360 |
| | GGATACGGTA GCAGCTACTG GTACTTCGAC GTGTGGGGCG CCGGAACCAC AGTGACCGTT | 420 |
| | TCGTCTGCCA GCACAAAAGG CCCTTCCGGC GGCGGAGGCT CCGGCGGCGG GGGCAGTGGC | 480 |
| | GGCGGCGGCT CTGGCGGCGG AGGCAGCGAC ATCGTGATGA CCCAGAGCCA CAAGTTCATG | 540 |
| | AGCACCAGCG TGGGCGACCG GGTGTCGATC ACCTGCAAGG CCAGTCAGGA TGTGAGCATC | 600 |
| | GCCGTCGCCT GGTATCAGCA AAAGCCCGGC CAAAGCCCTA AGGTGCTGAT CTATAGCGCC | 660 |
| | TCCTACAGAT ACACCGGCGT TCCTGACAGA TTCACAGGGT CTGGCAGCGG CACCGATTTC | 720 |
| | ACCTTCACAA TCTCCAGAGT GCAGGCCGAG GACCTGGCCG TGTACTACTG CCAGCAGCAC | 780 |
| | TACATCACAC CTCTGACCTT TGGTGCTGGC ACAAAGCTGG AACTGAAAAG AACCGTGGCC | 840 |
| | TTCAGAACTA AGCCTGCTGC CCTCGGCAAG GACACCATCC CTTGGCTGGG CCACCTGCTG | 900 |
| | GTGGGCCTGA GCGGAGCTTT TGGCTTCATC ATCCTGGTCT ACCTGCTGAT TAACTGCAGA | 960 |
| | AACACCGGTC CTTGGCTGAA GAAAGTGCTG AAGTGCAACA CCCCCGACCC TAGCAAGTTC | 1020 |
| | TTCAGCCAGC TGAGCAGCGA GCACGGCGGG GACGTGCAAA AATGGCTGAG CAGCCCCTTC | 1080 |
| | CCATCTTCCA GCTTCAGCCC TGGCGGCCTG GCTCCTGAGA TCAGCCCCCT GGAAGTGCTG | 1140 |
| | GAACGGGATA AGGTGACCCA GCTGTTACTC CAGCAGGACA AGGTGCCCGA ACCTGCCAGC | 1200 |
| | CTGAGCAGCA ACCACAGCCT GACTAGCTGC TTCACCAACC AGGGCTACTT CTTCTTTCAC | 1260 |
| | CTGCCCGACG CCCTGGAGAT CGAGGCCTGC CAGGTGTACT TCACCTACGA CCCTTACAGC | 1320 |
| | GAGGAGGACC CTGACGAGGG CGTGGCCGGG GCTCCTACCG GCTCGTCTCC TCAGCCTTTG | 1380 |
| | CAGCCTCTGA GCGGAGAGGA TGACGCCTAT TGCACCTTTC CTAGCAGAGA TGATCTGCTG | 1440 |
| | CTCTTCAGCC CTTCTCTGCT GGGCGGACCA AGCCCACCTT CTACCGCACC TGGCGGCTCT | 1500 |
| | GGCGCAGGCG AAGAGCGGAT GCCTCCTTCT CTGCAGGAGA GAGTGCCCCG GGACTGGGAC | 1560 |
| | CCTCAGCCGC TGGGACCTCC TACCCCTGGC GTGCCGGATC TGGTGGACTT TCAGCCACCA | 1620 |
| | CCTGAGCTGG TGCTGAGAGA AGCCGGCGAA GAGGTGCCTG ACGCCGGCCC TAGGGAGGGC | 1680 |
| | GTGAGCTTCC CTTGGAGCCG GCCTCCTGGA CAAGGCGAGT TCCGCGCCCT GAACGCCAGA | 1740 |
| | CTGCCCCTGA ACACCGACGC CTACCTGAGC CTGCAGGAGC TGCAGGGCCA GGACCCCACC | 1800 |
| | CACCTGGTGG GCAGCGGAGA GGGAAGAGGC AGCCTGTTGA CATGTGGCGA TGTCGAGGAA | 1860 |
| | AACCCTGGGC CTATGGCCCT GCCAGTGACA GCACTACTCC TGCCTCTGGC CCTGCTGCTC | 1920 |
| | CACGCCGCCA GACCTCAGGT GCAGCTGCAG CAATCTGGCC CCGAGCTAGT GCGGCCTGGC | 1980 |
| | ACCAGCGTGC GGATTTCCTG CAAGGCCTCT GGCTACACCT TCACAATCTA TTGGCTGGGC | 2040 |
| | TGGGTGAAGC AGAGACCTGG ACATGGGCTG GAGTGGATAG GAAACATCTT CCCTGGCTCA | 2100 |
| | GCTTACATCA ACTACAACGA GAAGTTTAAG GGAAAAGCCA CCCTGACAGC CGACACCAGC | 2160 |
| | AGCAGCACCG CCTATATGCA GCTGTCTAGC CTGACATCTG AGGATAGCGC CGTTTACTTC | 2220 |
| | TGCGCCAGAG AAGGCTCCAA TTCTGGCTAC TGGGGCCAGG GCACAACCTT AACCGTGTCC | 2280 |
| | AGCGGAGGAG GCGGCTCTGG CGGCGGCGGT TCAGGCGGGG GCGGAAGCGG CGGTGGAGGC | 2340 |
| | TCTGACATTG TGATGACCCA GAGCCCCAGC AGCCTGAGCG TGTCTGCTGG CGAGAAGGTG | 2400 |
| | ACCATGACCT GTAAATCTAG CCAGAGCCTG CTGAACAGCG GCAACCAGCA GAACTACCTG | 2460 |
| | GCCTGGTATC AGCAGAAACC TGGCCAGCCC CCCAAGCTGC TGATCTACGG CGCCAGCACC | 2520 |
| | AGAGAAAGCG GCGTGCCTGA CAGATTCACT GGCAGCGGCA GCGGCACAGA CTTCACCCTG | 2580 |
| | ACCATCAATA GCGTGCAGGC CGAAGATCTG GCTGTGTACT ACTGTCAGAG CGACCACATC | 2640 |
| | TACCCTTACA CCTTCGGCGG AGGCACCAAG CTAGAAATCA AGAGCAAGGA AAATCCATTT | 2700 |
| | CTGTTTGCCC TGGAAGCCGT GGTGATCAGC GTGGGAAGCA TGGGCCTGAT CATCTCGCTG | 2760 |
| | CTGTGCGTGT ACTTCTGGCT GGAAAGGACC ATGCCCCGCA TCCCTACCCT CAAGAACCTG | 2820 |
| | GAGGACCTGG TCACAGAGTA CCACGGCAAT TTCAGCGCCT GGTCCGGCGT GTCCAAGGGC | 2880 |
| | CTGGCCGAGA GCCTGCAACC CGATTACAGT GAACGGCTGT GTCTGGTGTC TGAGATCCCC | 2940 |
| | CCGAAGGGAG GCGCCCTGGG AGAAGGCCCA GGCGCCAGCC CTTGTAATCA GCATAGCCCT | 3000 |
| | TACTGGGCTC CTCCATGTTA CACCCTGAAG CCCGAAACC | 3039 |
| SEQ ID NO: 684 | ATGGCCCTGC CTGTGACCGC GCTGCTGCTG CCTCTGGCCC TGCTGCTGCA TGCCGCCAGG | 60 |
| CCR12.2: TROP2- | CCACAGATCC AGCTGGTGCA GAGCGGACCT GAACTGAAGA AGCCTGGGGA AACCGTGAAG | 120 |
| IL-18R | ATCAGCTGCA AGGCCTCCGG CTACACCTTC ACAAACTACG GCATGAACTG GGTGAAGCAG | 180 |
| | GCCCCTGGAA AGGGCCTGAA ATGGATGGGC TGGATCAACA CCAAGACCGG CGAGCCTACC | 240 |
| | TACGCCGAAG AGTTCAAGGG CAGATTCGCC TTCAGCCTGG AGACCTCAGC CAGCACCGCC | 300 |
| | TACCTGCAGA TCAACAACCT GAAGAAGGAA GATACCGCCA CCTACTTCTG CGGCCGGGGC | 360 |
| | GGTTATGGCA GCAGCTACTG GTACTTCGAC GTGTGGGGCG CCGGCACAAC AGTGACAGTA | 420 |
| | TCCAGCGCCT CCACAAAAGG ACCTAGCGGC GGCGGCGGCA GCGGTGGTGG CGGAAGTGGC | 480 |
| | GGCGGCGGCA GCGGCGGCGG TGGAAGCGAC ATCGTGATGA CCCAGAGCCA TAAGTTCATG | 540 |
| | AGCACAAGCG TGGGCGATCG GGTGAGCATC ACCTGCAAGG CCTCTCAGGA CGTGAGCATC | 600 |
| | GCCGTGGCCT GGTATCAGCA GAAACCTGGG CAGTCCCCTA AGGTGTTGAT CTATTCTGCT | 660 |
| | TCCTACCGAT ACACAGGAGT GCCTGACCGG TTCACCGGCT CTGGCAGCGG CACCGATTTC | 720 |
| | ACCTTTACAA TCAGCAGAGT GCAGGCTGAG GACCTGGCCG TGTACTACTG TCAGCAGCAC | 780 |
| | TACATCACCC CTCTGACCTT TGGCGCCGGA ACTAAACTGG AACTGAAGCG GACCGTGGCC | 840 |
| | GATATGGCTG ATATACCCGG CCACGTGTTC ACTAGAGGCA TGATCATTGC CGTGTTGATC | 900 |
| | CTGGTGGCCG TGGTTTGCCT GGTGACCGTG TGCGTGATCT ACAGAGTGGA CCTGGTCCTG | 960 |
| | TTCTACAGAC ACCTGACCAG ACGCGACGAG ACCCTGACAG ACGGCAAAAC ATACGACGCC | 1020 |
| | TTCGTGTCCT ACCTGAAAGA GTGCAGACCT GAGAATGGCG AGGAACACAC CTTTGCCGTC | 1080 |
| | GAGATCCTGC CCAGAGTGCT GGAAAAGCAC TTCGGCTATA AGCTGTGTAT CTTCGAGCGG | 1140 |
| | GACGTGGTGC CAGGCGGCGC CGTGGTGGAC GAGATCCACA GCCTGATTGA GAAAAGCCGG | 1200 |
| | CGGCTGATCA TCGTGCTGAG CAAGTCGTAC ATGAGCAATG AAGTGCGGTA CGAGCTGGAA | 1260 |
| | AGCGGCCTGC ACGAGGCCCT GGTCGAGAGA AAGATTAAGA TCATCCTGAT CGAGTTTACA | 1320 |
| | CCCGTGACAG ACTTTACCTT CCTGCCCCAG AGCCTGAAAC TGCTGAAGAG CCATAGAGTG | 1380 |
| | CTGAAGTGGA AGGCTGATAA GTCTCTGAGC TACAACTCTC GCTTCTGGAA GAACCTGCTC | 1440 |
| | TACCTGATGC CTGCCAAGAC AGTTAAGCCC GGCAGAGACG AGCCCGAGGT GCTGCCTGTG | 1500 |
| | CTTTCCGAGA GCGGATCTGG CGAAGGCAGA GGTTCCCTGC TTACATGTGG CGACGTGGAG | 1560 |
| | GAAAATCCTG GCCCTATGGC CCTGCCTGTT ACCGCTCTGC TGCTGCCTCT GGCCCTGCTG | 1620 |
| | CTGCACGCCG CTAGACCTCA GGTGCAACTG CAACAGAGCG GCCCTGAACT GGTCAGACCC | 1680 |
| | GGAACCTCCG TGCGGATCAG TTGCAAGGCC AGCGGCTACA CCTTCACCAT CTACTGGCTG | 1740 |
| | GGATGGGTTA AGCAGAGACC TGGCCACGGC TTGGAGTGGA TCGGCAACAT CTTTCCCGGC | 1800 |
| | TCCGCCTATA TCAACTACAA CGAGAAATTC AAAGGGAAGG CGACCCTGAC AGCCGACACC | 1860 |
| | AGCAGTTCCA CCGCCTACAT GCAGCTGTCC AGCCTGACAT CTGAGGACAG CGCAGTGTAT | 1920 |
| | TTTTGCGCCA GAGAAGGCAG CAACAGCGGA TACTGGGGAC AGGGCACCAC ACTCACCGTG | 1980 |
| | AGCAGCGGAG GCGGCGGCAG CGGCGGCGGC GGAAGCGGCG GCGGCGGGAG CGGCGGCGGC | 2040 |
| | GGATCTGACA TCGTGATGAC CCAAAGCCCT AGCAGCCTGA GTGTGAGCGC CGGCGAGAAG | 2100 |
| | GTGACCATGA CCTGTAAAAG CAGCCAGTCG CTGCTGAACA GCGGCAATCA GCAGAACTAC | 2160 |
| | CTGGCATGGT ATCAGCAAAA GCCCGGCCAG CCTCCAAAGC TGCTTATCTA CGGTGCCAGC | 2220 |
| | ACCCGGGAGA GCGGCGTTCC TGATCGGTTC ACTGGAAGTG GCAGCGGAAC CGACTTCACA | 2280 |
| | CTGACGATCA ATAGCGTGCA GGCCGAGGAC CTGGCCGTGT ACTACTGCCA AAGCGACCAC | 2340 |
| | ATCTACCCCT ACACCTTCGG GGGGGGCACC AAGCTCGAAA TCAAGAACAC AACACAGTCT | 2400 |
| | GTGCAGCTGA AAGAGAAGAG AGGCGTGGTT CTGCTGTACA TCCTTCTTGG AACCATCGGC | 2460 |
| | ACCCTGGTGG CCGTGTTAGC CGCCTCTGCT CTGCTGTACC GGCACTGGAT CGAGATCGTG | 2520 |
| | CTGCTGTATA GAACATACCA GTCTAAGGAC CAGACCCTGG GCGACAAGAA AGATTTCGAT | 2580 |
| | GCCTTCGTGT CCTACGCTAA GTGGAGCAGC TTCCCTTCTG AAGCCACCTC TAGCCTGTCT | 2640 |
| | GAAGAGCACC TGGCCCTGAG CCTGTTTCCA GATGTGCTGG AGAACAAGTA CGGATACAGC | 2700 |
| | CTGTGCCTGC TGGAAAGAGA CGTGGCCCCT GGAGGCGTGT ACGCCGAAGA CATCGTCAGC | 2760 |
| | ATCATCAAGC GGAGCAGAAG AGGCATCTTC ATCCTGAGCC CAAACTACGT GAACGGCCCC | 2820 |
| | AGCATCTTCG AGCTGCAGGC CGCCGTGAAC CTGGCTCTTG ATGACCAAAC ACTGAAGCTG | 2880 |
| | ATCCTGATCA AGTTCTGCTA CTTTCAGGAG CCTGAATCCC TGCCGCACCT GGTGAAGAAG | 2940 |
| | GCCCTGAGAG TTCTGCCCAC CGTGACCTGG CGCGGACTGA AATCTGTTCC TCCTAATAGC | 3000 |
| | AGGTTCTGGG CCAAGATGAG ATACCACATG CCAGTGAAGA ACAGCCAGGG GTTCACCTGG | 3060 |
| | AACCAGCTGA GAATCACAAG CAGAATCTTC CAGTGGAAGG GCCTGTCTCG AACAGAGACC | 3120 |
| | ACCGGCAGAA GCAGCCAACC TAAGGAATGG | 3150 |

Vectors encoding the CCRs designated CCR7.2. CCR8.2, CCR11.2, and CCR12.2 were prepared, and are presented in Table 89.

TABLE 89
Nucleotide sequences of exemplary vectors encoding the biepitope CCRs
designated CCR7.2, CCR8.2, CCR11.2, and CCR12.2.

| Identifier | Sequence (One-Letter Nucleotide Symbols) | |
|---|---|---|
| SEQ ID NO:685 | GTCGACGGAT CGGGAGATCT CCCGATCCCC TATGGTGCAC TCTCAGTACA ATCTGCTCTG | 60 |
| CCR7.2: chPD-L1- | ATGCCGCATA GTTAAGCCAG TATCTGCTCC CTGCTTGTGT GTTGGAGGTC GCTGAGTAGT | 120 |
| IL-2R | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGASTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCT TGCCCGTCAC TGCCTTGCTG CTGCCTCTGG CCTTACTGCT | 2940 |
| | GCACGCCGCC AGACCTAGCT ACGTGCTGAC CCAGCCTCCT AGCGTGAGCG TGGCCCCTGG | 3000 |
| | CCAGACCGCT AGAATCACCT GCGGCGGCAA CAACATCGGA AGAAAGATCG TGCACTGGTA | 3060 |
| | TCAGCAGCGG CCTGGCCAAG CTCCTGTGCT GGTGATTTAC TACGACACCG ACAGACCAGC | 3120 |
| | CGGTATCCCC GAGCGGTTCA GCGGCTCTAA CAGCGGAAAT ATGGCGACAC TGACCATCTC | 3180 |
| | CACCGTGGGC GCCGGCGATG AGGCCGACTA CTACTGCCAA GTGTGGGATA CAGGCTCTGA | 3240 |
| | CCACGTGGTG TTCGGCGGGG GCACCAAGCT GACCGTGCTG GGAGGCGGCG GCAGCGGCGG | 3300 |
| | CGGCGGCAGC GGCGGCGGCG GCAGCGGCGG AGGAGGAAGC GAGGTGCAAC TGGTCGAATC | 3360 |
| | CGGCGGAGGG CTGGTACAGC CTGGTGGCTC CTTGCGGCTG AGCTGCGCCG CTTCTGGCTT | 3420 |
| | CACCTTCAGC AATTACGCCA TGAGCTGGGT TAGACAGGCC CCTGGGAAGG GCCTGGAATG | 3480 |
| | GGTGAGCACC ATCTCAGGCA GCGGAGGCAC AACCTACTAC GCCGATAGCG TGAAAGGCAG | 3540 |
| | GTTCACAATC AGCAGAGATA ATAGCAAAAA CACCCTGTAC CTGCAAATGA ACAGCCTGAG | 3600 |
| | AGTGGAAGAT ACAGCCGTGT ATTACTGTGC CAAGGACTGG TTCAGAAGCT CCAGCCCTGA | 3660 |
| | CGCCTTTGAC ATTTGGGGCC AGGGGACCAC CGTGACCGTG AGTGCTTTCC GGACCAAGCC | 3720 |
| | TGCCGCCCTG GGAAAAGACA CCATCCCTTG GCTGGGCCAC CTGCTAGTGG GCCTTTCGGG | 3780 |
| | AGCCTTCGGC TTCATCATCC TGGTGTACCT GCTGATCAAC TGCAGAAACA CCGGCCCTTG | 3840 |
| | GCTGAAAAAG GTGCTCAAGT GCAACACACC TGACCCTAGC AAGTTCTTCT CTCAGCTGAG | 3900 |
| | CAGCGAGCAC GGAGGCGATG TGCAGAAATG GCTGAGCAGC CCCTTCCCTA GCAGCTCTTT | 3960 |
| | CAGCCCTGGA GGCCTGGCCC CTGAAATCTC TCCTCTGGAA GTGCTGGAGA GAGACAAGGT | 4020 |
| | GACCCAGCTG CTGCTGCAGC AAGACAAGGT GCCCGAACCC GCCAGCCTGA GCAGCAACCA | 4080 |
| | CAGCCTGACC AGCTGTTTTA CAAATCAGGG CTACTTCTTC TTCCACCTGC CTGATGCCCT | 4140 |
| | GGAGATCGAG GCCTGTCAGG TGTACTTCAC CTACGATCCC TACTCCGAGG AGGACCCTGA | 4200 |
| | CGAGGGCGTG GCCGGTGCTC CAACAGGCAG CTCGCCTCAG CCTCTGCAGC CTCTCAGCGG | 4260 |
| | CGAGGACGAC GCTTATTGCA CCTTTCCTAG CAGAGACGAC CTGCTGCTTT TCAGCCCCTC | 4320 |
| | CCTGCTCGGC GGCCCCTCCC CCCCCAGCAC CGCCCCAGGC GGAAGCGGCG CCGGCGAGGA | 4380 |
| | GCGGATGCCT CCTTCTCTGC AGGAGAGAGT GCCTCGGGAT TGGGACCCTC AGCCTCTGGG | 4440 |
| | ACCTCCTACC CCTGGCGTGC CCGACCTGGT GGACTTCCAG CCCCCTCCCG AGCTGGTCCT | 4500 |
| | GAGAGAAGCC GGCGAGGAAG TGCCCGACGC CGGACCTCGG GAAGGCGTGT CCTTCCCCTG | 4560 |
| | GTCCCGGCCT CCAGGCCAGG GCGAGTTCAG AGCGCTGAAT GCCAGACTGC CACTGAACAC | 4620 |
| | CGACGCCTAC CTGAGCCTGC AGGAGCTGCA GGGCCAGGAC CCGACCCACC TGGTGGGCTC | 4680 |
| | TGGCGAAGGC CGGGGCAGCC TGCTGACCTG TGGCGATGTG GAAGAGAACC CTGGCCCTAT | 4740 |
| | GGCTCTCCCT GTTACAGCCC TGCTTCTGCC TCTGGCTCTC CTGCTGCATG CTGCGCGGCC | 4800 |
| | CAACTTTATG CTGACCCAGC CCCACAGCGT CAGTGAAAGC CTGGGCAAAA CCGTGACAAT | 4860 |
| | CAGCTGCACA GGCAGCTCAG GAAGCATCGC CAGGAAGTTC GTGCAGTGGT ATCAGCAACG | 4920 |
| | GCCAGGATCA TCTCCTACAA CCGTGATCTA CGAGAACAAC CAGAGACCTT CTGGCGTGAG | 4980 |
| | TGATAGATTC AGCGGCTCTA TCGGCAGTAG CTCTAATAGC GCCTCTCTGA CAATCAGCGG | 5040 |
| | CCTGAAGACC GAGGATGAGG CCGATTACTA TTGCCAGTCC TACGACAGCA GCAACGTGGT | 5100 |
| | GTTCGGCGGC GGCACAAAGG TTACCGTCCT GGGCGGCGGA GGCTCTGGCG GCGGCGGCAG | 5160 |
| | CGGCGGCGGA GGCTCTGGCG GCGGCGGTAG CCAGGTGCAA CTGCAAGAAT CCGGAGGCGG | 5220 |
| | CCTGGTCAAG CCTGGTGGGA GCCTGAGACT GAGCTGCGCC GCCAGCGGCT TCACATTCTC | 5280 |
| | CAGCTACAGC ATGAACTGGG TCCGGCAGGC CCCAGGCAAG GGCCTGGAGT GGGTCAGCGG | 5340 |
| | AATCAACACC GCCGGCGACA CACATTACCC TGAGAGCGTG AAGGGCAGAT TTACCATCAG | 5400 |
| | CAGAGACAAC GCCCGGAACA GCCTGAACCT GCAGATGAAT AGCCTGCGGG CCGAAGATAC | 5460 |
| | AGCCGTTTAC TACTGCGTGA GAGAGAGAGT GGAACGCGAG TACAGCGGAT ATGACGCCTT | 5520 |
| | CGACATCTGG GGCCAGGGAA CGACCGTGAC AGTGTCGGCC AGCAAGGAAA ATCCTTTTCT | 5580 |
| | GTTCGCCCTG GAGGCCGTCG TAATCAGCGT GGGAAGCATG GGCCTGATCA TCAGCCTGCT | 5640 |
| | GTGTGTGTAC TTTTGGCTGG AGCGTACCAT GCCTAGAATC CCCACCCTGA AGAACCTGGA | 5700 |
| | GGACCTGGTG ACAGAATACC ACGGCAACTT CAGCGCTTGG AGCGGTGTGT CTAAGGGACT | 5760 |
| | GGCCGAGTCT CTGCAGCCAG ACTACAGCGA GCGGCTGTGC CTGGTGAGCG AAATCCCACC | 5820 |
| | AAAGGGAGGG GCTCTCGGCG AAGGCCCTGG CGCTTCCCCT TGTAACCAGC ACTCTCCCTA | 5880 |
| | CTGGGCTCCT CCATGCTACA CCCTGAAGCC TGAAACCTGA GAATTCGATA TCAAGCTTAT | 5940 |
| | CGGTAATCAA CCTCTGGATT ACAAAATTTG TGAAAGATTG ACTGGTATTC TTAACTATGT | 6000 |
| | TGCTCCTTTT ACGCTATGTG GATACGCTGC TTTAATGCCT TTGTATCATG CTATTGCTTC | 6060 |
| | CCGTATGGCT TTCASTTTCT CCTCCTTGTA TAAATCCTGG TTGCTGTCTC TTTATGAGGA | 6120 |
| | GTTGTGGCCC GTTGTCAGGC AACGTGGCGT GGTGTGCACT GTGTTTGCTG ACGCAACCCC | 6180 |
| | CACTGGTTGG GGCATTGCCA CCACCTGTCA GCTCCTTTCC GGGACTTTCG CTTTCCCCCT | 6240 |
| | CCCTATTGCC ACGGCGGAAC TCATCGCCGC CTGCCTTGCC CGCTGCTGGA CAGGGGCTCG | 6300 |
| | GCTGTTGGGC ACTGACAATT CCGTGGTGTT GTCGGGGAAA TCATCGTCCT TTCCTTGGCT | 6360 |
| | GCTCGCCTGT GTTGCCACCT GGATTCTGCG CGGGACGTCC TTCTGCTACG TCCCTTCGGC | 6420 |
| | CCTCAATCCA GCGGACCTTC CTTCCCGCGG CCTGCTGCCG GCTCTGCGGC CTCTTCCGCG | 6480 |
| | TCTTCGCCTT CGCCCTCAGA CGAGTCGGAT CTCCCTTTGG GCCGCCTCCC CGCATCGATA | 6540 |
| | CCGTCGACCT CGAGACCTAG AAAAACATGG AGCAATCACA AGTAGCAATA CAGCAGCTAC | 6600 |
| | CAATGCTGAT TGTGCCTGGC TAGAAGCACA AGAGGAGGAG GAGGTGGGTT TTCCAGTCAC | 6660 |
| | ACCTCAGGTA CCTTTAAGAC CAATGACTTA CAAGGCAGCT GTAGATCTTA GCCACTTTTT | 6720 |
| | AAAAGAAAAG GGGGGACTGG AAGGGCTAAT TCACTCCCAA CGAAGACAAG ATATCCTTGA | 6780 |
| | TCTGTGGATC TACCACACAC AAGGCTACTT CCCTGATTGG CAGAACTACA CACCAGGGCC | 6840 |
| | AGGGATCAGA TATCCACTGA CCTTTGGATG GTGCTACAAG CTAGTACCAG TTGAGCAAGA | 6900 |
| | GAAGGTAGAA GAAGCCAATG AAGGAGAGAA CACCCGCTTG TTACACCCTG TGAGCCTGCA | 6960 |
| | TGGGATGGAT GACCCGGAGA GAGAAGTATT AGAGTGGAGG TTTGACAGCC GCCTAGCATT | 7020 |
| | TCATCACATG GCCCGAGAGC TGCATCCGGA CTGTACTGGG TCTCTCTGGT TAGACCAGAT | 7080 |
| | CTGAGCCTGG GAGCTCTCTG GCTAACTAGG GAACCCACTG CTTAAGCCTC AATAAAGCTT | 7140 |
| | GCCTTGAGTG CTTCAAGTAG TGTGTGCCCG TCTGTTGTGT GACTCTGGTA ACTAGAGATC | 7200 |
| | CCTCAGACCC TTTTAGTCAG TGTGGAAAAT CTCTAGCAGG GCCCGTTTAA ACCCGCTGAT | 7260 |
| | CAGCCTCGAC TGTGCCTTCT AGTTGCCAGC CATCTGTTGT TTGCCCCTCC CCCGTGCCTT | 7320 |
| | CCTTGACCCT GGAAGGTGCC ACTCCCACTG TCCTTTCCTA ATAAAATGAG GAAATTGCAT | 7380 |
| | CGCATTGTCT GAGTAGGTGT CATTCTATTC TGGGGGGTGG GGTGGGGCAG GACAGCAAGG | 7440 |
| | GGGAGGATTG GGAAGACAAT AGCAGGCATG CTGGGGATGC GGTGGGCTCT ATGGCTTCTG | 7500 |
| | AGGCGGAAAG AACCAGCTGG GGCTCTAGGG GGTATCCCCA CGCGCCCTGT AGCGGCGCAT | 7560 |
| | TAAGCGCGGC GGGTGTGGTG GTTACGCGCA GCGTGACCGC TACACTTGCC AGCGCCCTAG | 7620 |
| | CGCCCGCTCC TTTCGCTTTC TTCCCTTCCT TTCTCGCCAC GTTCGCCGGC TTTCCCCGTC | 7680 |
| | AAGCTCTAAA TCGGGGGCTC CCTTTAGGGT TCCGATTTAG TGCTTTACGG CACCTCGACC | 7740 |
| | CCAAAAAACT TGATTAGGGT GATGGTTCAC GTAGTGGGCC ATCGCCCTGA TAGACGGTTT | 7800 |
| | TTCGCCCTTT GACGTTGGAG TCCACGTTCT TTAATAGTGG ACTCTTGTTC CAAACTGGAA | 7860 |
| | CAACACTCAA CCCTATCTCG GTCTATTCTT TTGATTTATA AGGGATTTTG CCGATTTCGG | 7920 |
| | CCTATTGGTT AAAAAATGAG CTGATTTAAC AAAAATTTAA CGCGAATTAA TTCTGTGGAA | 7980 |
| | TGTGTGTCAG TTAGGGTGTG GAAAGTCCCC AGGCTCCCCA GCAGGCAGAA GTATGCAAAG | 8040 |
| | CATGCATCTC AATTAGTCAG CAACCAGGTG TGGAAAGTCC CCAGGCTCCC CAGCAGGCAG | 8100 |
| | AAGTATGCAA AGCATGCATC TCAATTAGTC AGCAACCATA GTCCCGCCCC TAACTCCGCC | 8160 |
| | CATCCCGCCC CTAACTCCGC CCAGTTCCGC CCATTCTCCG CCCCATGGCT GACTAATTTT | 8220 |
| | TTTTATTTAT GCAGAGGCCG AGGCCGCCTC TGCCTCTGAG CTATTCCAGA AGTAGTGAGG | 8280 |
| | AGGCTTTTTT GGAGGCCTAG GCTTTTGCAA AAAGCTCCCG GGAGCTTGTA TATCCATTTT | 8340 |
| | CGGATCTGAT CAGCACGTGT TGACAATTAA TCATCGGCAT AGTATATCGG CATAGTATAA | 8400 |
| | TACGACAAGG TGAGGAACTA AACCATGGCC AAGTTGACCA GTGCCGTTCC GGTGCTCACC | 8460 |
| | GCGCGCGACG TCGCCGGAGC GGTCGAGTTC TGGACCGACC GGCTCGGGTT CTCCCGGGAC | 8520 |
| | TTCGTGGAGG ACGACTTCGC CGGTGTGGTC CGGGACGACG TGACCCTGTT CATCAGCGCG | 8580 |
| | GTCCAGGACC AGGTGGTGCC GGACAACACC CTGGCCTGGG TGTGGGTGCG CGGCCTGGAC | 8640 |
| | GAGCTGTACG CCGAGTGGTC GGAGGTCGTG TCCACGAACT TCCGGGACGC CTCCGGGCCG | 8700 |
| | GCCATGACCG AGATCGGCGA GCAGCCGTGG GGGCGGGAGT TCGCCCTGCG CGACCCGGCC | 8760 |
| | GGCAACTGCG TGCACTTCGT GGCCGAGGAG CAGGACTGAC ACGTGCTACG AGATTTCGAT | 8820 |
| | TCCACCGCCG CCTTCTATGA AAGGTTGGGC TTCGGAATCG TTTTCCGGGA CGCCGGCTGG | 8880 |
| | ATGATCCTCC AGCGCGGGGA TCTCATGCTG GAGTTCTTCG CCCACCCCAA CTTGTTTATT | 8940 |
| | GCAGCTTATA ATGGTTACAA ATAAAGCAAT AGCATCACAA ATTTCACAAA TAAAGCATTT | 9000 |
| | TTTTCACTGC ATTCTAGTTG TGGTTTGTCC AAACTCATCA ATGTATCTTA TCATGTCTGT | 9060 |
| | ATACCGTCGA CCTCTAGCTA GAGCTTGGCG TAATCATGGT CATAGCTGTT TCCTGTGTGA | 9120 |
| | AATTGTTATC CGCTCACAAT TCCACACAAC ATACGAGCCG GAAGCATAAA GTGTAAAGCC | 9180 |
| | TGGGGTGCCT AATGAGTGAG CTAACTCACA TTAATTGCGT TGCGCTCACT GCCCGCTTTC | 9240 |
| | CAGTCGGGAA ACCTGTCGTG CCAGCTGCAT TAATGAATCG GCCAACGCGC GGGGAGAGGC | 9300 |
| | GGTTTGCGTA TTGGGCGCTC TTCCGCTTCC TCGCTCACTG ACTCGCTGCG CTCGGTCGTT | 9360 |
| | CGGCTGCGGC GAGCGGTATC AGCTCACTCA AAGGCGGTAA TACGGTTATC CACAGAATCA | 9420 |
| | GGGGATAACG CAGGAAAGAA CATGTGAGCA AAAGGCCAGC AAAAGGCCAG GAACCGTAAA | 9480 |
| | AAGGCCGCGT TGCTGGCGTT TTTCCATAGG CTCCGCCCCC CTGACGAGCA TCACAAAAAT | 9540 |
| | CGACGCTCAA GTCAGAGGTG GCGAAACCCG ACAGGACTAT AAAGATACCA GGCGTTTCCC | 9600 |
| | CCTGGAAGCT CCCTCGTGCG CTCTCCTGTT CCGACCCTGC CGCTTACCGG ATACCTGTCC | 9660 |
| | GCCTTTCTCC CTTCGGGAAG CGTGGCGCTT TCTCATAGCT CACGCTGTAG GTATCTCAGT | 9720 |
| | TCGGTGTAGG TCGTTCGCTC CAAGCTGGGC TGTGTGCACG AACCCCCCGT TCAGCCCGAC | 9780 |
| | CGCTGCGCCT TATCCGGTAA CTATCGTCTT GAGTCCAACC CGGTAAGACA CGACTTATCG | 9840 |
| | CCACTGGCAG CAGCCACTGG TAACAGGATT AGCAGAGCGA GGTATGTAGG CGGTGCTACA | 9900 |
| | GAGTTCTTGA AGTGGTGGCC TAACTACGGC TACACTAGAA GAACAGTATT TGGTATCTGC | 9960 |
| | GCTCTGCTGA AGCCAGTTAC CTTCGGAAAA AGAGTTGGTA GCTCTTGATC CGGCAAACAA | 10020 |
| | ACCACCGCTG GTAGCGGTGG TTTTTTTGTT TGCAAGCAGC AGATTACGCG CAGAAAAAAA | 10080 |
| | GGATCTCAAG AAGATCCTTT GATCTTTTCT ACGGGGTCTG ACGCTCAGTG GAACGAAAAC | 10140 |
| | TCACGTTAAG GGATTTTGGT CATGAGATTA TCAAAAAGGA TCTTCACCTA GATCCTTTTA | 10200 |
| | AATTAAAAAT GAAGTTTTAA ATCAATCTAA AGTATATATG AGTAAACTTG GTCTGACAGT | 10260 |
| | TACCAATGCT TAATCAGTGA GGCACCTATC TCAGCGATCT GTCTATTTCG TTCATCCATA | 10320 |
| | GTTGCCTGAC TCCCCGTCGT GTAGATAACT ACGATACGGG AGGGCTTACC ATCTGGCCCC | 10380 |
| | AGTGCTGCAA TGATACCGCG AGACCCACGC TCACCGGCTC CAGATTTATC AGCAATAAAC | 10440 |
| | CAGCCAGCCG GAAGGGCCGA GCGCAGAAGT GGTCCTGCAA CTTTATCCGC CTCCATCCAG | 10500 |
| | TCTATTAATT GTTGCCGGGA AGCTAGAGTA AGTAGTTCGC CAGTTAATAG TTTGCGCAAC | 10560 |
| | GTTGTTGCCA TTGCTACAGG CATCGTGGTG TCACGCTCGT CGTTTGGTAT GGCTTCATTC | 10620 |
| | AGCTCCGGTT CCCAACGATC AAGGCGAGTT ACATGATCCC CCATGTTGTG CAAAAAAGCG | 10680 |
| | GTTAGCTCCT TCGGTCCTCC GATCGTTGTC AGAAGTAAGT TGGCCGCAGT GTTATCACTC | 10740 |
| | ATGGTTATGG CAGCACTGCA TAATTCTCTT ACTGTCATGC CATCCGTAAG ATGCTTTTCT | 10800 |
| | GTGACTGGTG AGTACTCAAC CAAGTCATTC TGAGAATAGT GTATGCGGCG ACCGAGTTGC | 10860 |
| | TCTTGCCCGG CGTCAATACG GGATAATACC GCGCCACATA GCAGAACTTT AAAAGTGCTC | 10920 |
| | ATCATTGGAA AACGTTCTTC GGGGCGAAAA CTCTCAAGGA TCTTACCGCT GTTGAGATCC | 10980 |
| | AGTTCGATGT AACCCACTCG TGCACCCAAC TGATCTTCAG CATCTTTTAC TTTCACCAGC | 11040 |
| | GTTTCTGGGT GAGCAAAAAC AGGAAGGCAA AATGCCGCAA AAAAGGGAAT AAGGGCGACA | 11100 |
| | CGGAAATGTT GAATACTCAT ACTCTTCCTT TTTCAATATT ATTGAAGCAT TTATCAGGGT | 11160 |
| | TATTGTCTCA TGAGCGGATA CATATTTGAA TGTATTTAGA AAAATAAACA AATAGGGGTT | 11220 |
| | CCGCGCACAT TTCCCCGAAA AGTGCCACCT GAC | 11253 |
| SEQ ID NO: 686 | GTCGACGGAT CGGGAGATCT CCCGATCCCC TATGGTGCAC TCTCAGTACA ATCTGCTCTG | 60 |
| CCR8.2: chPD-L1- | ATGCCGCATA GTTAAGCCAG TATCTGCTCC CTGCTTGTGT GTTGGAGGTC GCTGAGTAGT | 120 |
| IL-18R | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGASTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TGCCAGTGAC AGCCCTGCTT CTGCCTCTGG CTCTGCTGCT | 2940 |
| | CCACGCCGCC AGACCCAGCT ACGTGCTCAC ACAGCCTCCT TCTGTGAGCG TGGCCCCAGG | 3000 |
| | ACAGACAGCC AGAATCACCT GTGGCGGCAA CAACATCGGC AGAAAGATCG TGCACTGGTA | 3060 |
| | TCAGCAAAGA CCTGGCCAGG CCCCTGTGCT GGTGATCTAC TACGATACTG ATCGGCCCGC | 3120 |
| | CGGCATCCCT GAGCGGTTTT CTGGCTCTAA CTCCGGAAAT ATGGCAACAC TGACAATCAG | 3180 |
| | CACAGTGGGC GCCGGAGACG AAGCTGACTA CTACTGCCAG GTGTGGGATA CTGGCTCGGA | 3240 |
| | CCACGTGGTT TTCGGCGGCG GCACCAAGCT GACCGTCCTG GGCGGCGGTG GTTCGGGCGG | 3300 |
| | CGGCGGCTCT GGCGGCGGCG GCTCTGGCGG AGGCGGCAGC GAGGTGCAGC TGGTTGAAAG | 3360 |
| | CGGCGGCGGT CTGGTGCAGC CTGGAGGCTC CTTACGGCTG TCCTGCGCCG CCAGCGGCTT | 3420 |
| | CACTTTCAGC AACTACGCCA TGAGCTGGGT CCGGCAGGCC CCTGGAAAAG GCCTGGAATG | 3480 |
| | GGTGAGCACC ATCAGCGGAA GCGGCGGGAC CACCTATTAC GCCGACAGCG TGAAGGGCAG | 3540 |
| | ATTTACCATC AGCAGGGACA ATAGCAAGAA TACCCTGTAC CTGCAGATGA ACAGCCTGCG | 3600 |
| | AGTGGAGGAC ACAGCCGTCT ACTACTGCGC CAAGGACTGG TTCAGAAGCA GCTCTCCTGA | 3660 |
| | CGCCTTCGAC ATCTGGGGCC AGGGCACCAC CGTGACTGTC AGCGCCGACA TGGCCGACAT | 3720 |
| | CCCTGGCCAC GTGTTTACCA GAGGCATGAT CATCGCCGTG CTGATTCTGG TGGCCGTGGT | 3780 |
| | TTGTCTGGTG ACCGTGTGCG TGATCTACCG GGTGGACCTG GTGCTGTTCT ACCGGCATCT | 3840 |
| | GACAAGAAGG GACGAAACCT TGACGGACGG CAAGACATAC GACGCATTCG TGTCTTACCT | 3900 |
| | GAAGGAGTGC AGACCCGAGA ACGGCGAAGA ACACACCTTT GCCGTGGAAA TCCTGCCTAG | 3960 |
| | AGTGCTGGAA AAGCACTTCG GCTACAAGCT GTGCATCTTC GAGAGAGATG TTGTGCCCGG | 4020 |
| | CGGAGCTGTC GTGGATGAGA TACACAGTCT GATCGAGAAA AGCAGAAGAC TGATTATCGT | 4080 |
| | GCTCTCCAAG AGCTATATGA GCAACGAGGT GAGATACGAG CTGGAGAGCG GTCTCCACGA | 4140 |
| | GGCTCTGGTC GAACGGAAGA TCAAGATTAT TCTGATCGAG TTCACCCCTG TTACAGACTT | 4200 |
| | CACCTTCCTG CCTCAGAGCC TGAAGTTACT GAAGAGTCAC AGAGTGCTGA AGTGGAAAGC | 4260 |
| | TGATAAGAGC CTGAGCTACA ACAGCCGCTT CTGGAAGAAT CTGCTCTACC TGATGCCTGC | 4320 |
| | CAAAACAGTG AAGCCTGGCA GGGATGAGCC TGAGGTGCTG CCCGTGCTGT CTGAAAGCGG | 4380 |
| | CTCCGGGGAG GGCAGAGGCT CCCTGCTGAC CTGCGGCGAC GTGGAAGAGA ACCCCGGACC | 4440 |
| | TATGGCTCTG CCAGTGACCG CTCTGCTGCT GCCTCTGGCC CTGCTGCTGC ATGCCGCCAG | 4500 |
| | GCCTAACTTC ATGCTGACCC AACCTCACAG CGTGTCGGAG TCTCTGGGCA AGACCGTGAC | 4560 |
| | AATCAGCTGC ACCGGAAGCT CTGGCAGCAT CGCCCGTAAA TTCGTGCAGT GGTATCAGCA | 4620 |
| | GAGGCCGGGC TCATCCCCTA CCACCGTGAT CTACGAGAAC AACCAGCGGC CTAGCGGCGT | 4680 |
| | GAGCGACAGA TTCAGCGGAA GCATCGGAAG CAGCAGCAAT AGCGCCTCAC TGACCATCAG | 4740 |
| | CGGCCTGAAA ACCGAGGACG AAGCCGACTA CTACTGTCAG AGCTACGACT CCAGCAACGT | 4800 |
| | GGTTTTCGGC GGCGGAACAA AGGTGACCGT GCTGGGCGGC GGCGGATCTG GTGGCGGCGG | 4860 |
| | CTCCGGCGGA GGCGGCTCTG GGGGCGGCGG TAGCCAAGTG CAGCTGCAGG AGAGCGGAGG | 4920 |
| | CGGCCTGGTG AAGCCAGGCG GCTCCCTGAG ACTGAGCTGT GCCGCTAGTG GCTTTACCTT | 4980 |
| | TAGCAGTTAC AGCATGAACT GGGTGAGACA GGCCCCCGGC AAGGGACTGG AATGGGTGTC | 5040 |
| | AGGCATCAAC ACCGCTGGCG ATACACACTA CCCCGAGAGC GTGAAAGGCA GATTCACAAT | 5100 |
| | CAGCAGAGAT AACGCCAGAA ACAGCCTGAA CCTGCAGATG AATAGCTTGC GGGCCGAGGA | 5160 |
| | CACCGCCGTG TACTACTGCG TGCGGGAACG GGTGGAAAGA GAGTACTCGG GATACGACGC | 5220 |
| | CTTCGACATC TGGGGACAGG GAACCACAGT GACAGTGTCC GCCAACACGA CCCAGAGCGT | 5280 |
| | ACAACTCAAA GAAAAGCGGG GAGTGGTCCT GCTGTATATC CTGCTCGGCA CTATCGGCAC | 5340 |
| | CCTGGTGGCC GTCCTGGCCG CCAGCGCCCT GCTGTATAGA CACTGGATTG AGATCGTGCT | 5400 |
| | CCTGTACAGA ACCTACCAGA GCAAAGACCA GACGCTGGGC GACAAGAAGG ATTTCGACGC | 5460 |
| | CTTTGTGAGC TACGCCAAAT GGTCCTCCTT CCCTAGCGAG GCCACATCTA GTCTGTCTGA | 5520 |
| | GGAACACCTG GCCCSGTCCC TTTTCCCCGA TGTGCTGGAG AACAAGTACG GCTACAGCCT | 5580 |
| | GTGCCTGCTG GAACGGGATG TGGCTCCTGG AGGCGTGTAC GCCGAAGACA TCGTGTCTAT | 5640 |
| | CATCAAAAGA AGCCGGAGAG GCATCTTCAT CCTGTCTCCA AACTACGTGA ACGGCCCTAG | 5700 |
| | CATCTTCGAG CTGCAAGCGG CTGTTAACTT GGCTCTGGAC GACCAGACCC TGAAGCTGAT | 5760 |
| | CCTGATCAAG TTCTGCTACT TCCAGGAGCC TGAGTCCCTG CCCCACCTGG TGAAGAAGGC | 5820 |
| | CCTCAGAGTG CTCCCAACCG TTACATGGCG GGGCCTGAAG AGCGTGCCCC CCAACTCCAG | 5880 |
| | ATTTTGGGCC AAGATGAGAT ACCACATGCC TGTGAAGAAT AGCCAAGGCT TCACCTGGAA | 5940 |
| | CCAGCTGCGG ATCACCAGCC GTATCTTCCA GTGGAAGGGC CTGTCTAGGA CCGAGACAAC | 6000 |
| | CGGCAGATCT AGCCAGCCTA AGGAATGGTG AGAATTCGAT ATCAAGCTTA TCGGTAATCA | 6060 |
| | ACCTCTGGAT TACAAAATTT GTGAAAGATT GACTGGTATT CTTAACTATG TTGCTCCTTT | 6120 |
| | TACGCTATGT GGATACGCTG CTTTAATGCC TTTGTATCAT GCTATTGCTT CCCGTATGGC | 6180 |
| | TTTCATTTTC TCCTCCTTGT ATAAATCCTG GTTGCTGTCT CTTTATGAGG AGTIGTGGCC | 6240 |
| | CGTTGTCAGG CAACGTGGCG TGGTGTGCAC TGTGTTTGCT GACGCAACCC CCACTGGTTG | 6300 |
| | GGGCATTGCC ACCACCTGTC AGCTCCTTTC CGGGACTTTC GCTTTCCCCC TCCCTATTGC | 6360 |
| | CACGGCGGAA CTCATCGCCG CCTGCCTTGC CCGCTGCTGG ACAGGGGCTC GGCTGTTGGG | 6420 |
| | CACTGACAAT TCCGTGGTGT TGTCGGGGAA ATCATCGTCC TTTCCTTGGC TGCTCGCCTG | 6480 |
| | TGTTGCCACC TGGASTCTGC GCGGGACGTC CTTCTGCTAC GTCCCTTCGG CCCTCAATCC | 6540 |
| | AGCGGACCTT CCTTCCCGCG GCCTGCTGCC GGCTCTGCGG CCTCTTCCGC GTCTTCGCCT | 6600 |
| | TCGCCCTCAG ACGAGTCGGA TCTCCCTTTG GGCCGCCTCC CCGCATCGAT ACCGTCGACC | 6660 |
| | TCGAGACCTA GAAAAACATG GAGCAATCAC AAGTAGCAAT ACAGCAGCTA CCAATGCTGA | 6720 |
| | TTGTGCCTGG CTAGAAGCAC AAGAGGAGGA GGAGGTGGGT TTTCCAGTCA CACCTCAGGT | 6780 |
| | ACCTTTAAGA CCAATGACTT ACAAGGCAGC TGTAGATCTT AGCCACTTTT TAAAAGAAAA | 6840 |
| | GGGGGGACTG GAAGGGCTAA TTCACTCCCA ACGAAGACAA GATATCCTTG ATCTGTGGAT | 6900 |
| | CTACCACACA CAAGGCTACT TCCCTGATTG GCAGAACTAC ACACCAGGGC CAGGGATCAG | 6960 |
| | ATATCCACTG ACCTTTGGAT GGTGCTACAA GCTAGTACCA GTTGAGCAAG AGAAGGTAGA | 7020 |
| | AGAAGCCAAT GAAGGAGAGA ACACCCGCTT GTTACACCCT GTGAGCCTGC ATGGGATGGA | 7080 |
| | TGACCCGGAG AGAGAAGTAT TAGAGTGGAG GTTTGACAGC CGCCTAGCAT TTCATCACAT | 7140 |
| | GGCCCGAGAG CTGCATCCGG ACTGTACTGG GTCTCTCTGG TTAGACCAGA TCTGAGCCTG | 7200 |
| | GGAGCTCTCT GGCTAACTAG GGAACCCACT GCTTAAGCCT CAATAAAGCT TGCCTTGAGT | 7260 |
| | GCTTCAAGTA GTGTGTGCCC GTCTGTTGTG TGACTCTGGT AACTAGAGAT CCCTCAGACC | 7320 |
| | CTTTTAGTCA GTGTGGAAAA TCTCTAGCAG GGCCCGTTTA AACCCGCTGA TCAGCCTCGA | 7380 |
| | CTGTGCCTTC TAGTTGCCAG CCATCTGTTG TTTGCCCCTC CCCCGTGCCT TCCTTGACCC | 7440 |
| | TGGAAGGTGC CACTCCCACT GTCCTTTCCT AATAAAATGA GGAAATTGCA TCGCATTGTC | 7500 |
| | TGAGTAGGTG TCATTCTATT CTGGGGGGTG GGGTGGGGCA GGACAGCAAG GGGGAGGATT | 7560 |
| | GGGAAGACAA TAGCAGGCAT GCTGGGGATG CGGTGGGCTC TATGGCTTCT GAGGCGGAAA | 7620 |
| | GAACCAGCTG GGGCTCTAGG GGGTATCCCC ACGCGCCCTG TAGCGGCGCA TTAAGCGCGG | 7680 |
| | CGGGTGTGGT GGTTACGCGC AGCGTGACCG CTACACTTGC CAGCGCCCTA GCGCCCGCTC | 7740 |
| | CTTTCGCTTT CTTCCCTTCC TTTCTCGCCA CGTTCGCCGG CTTTCCCCGT CAAGCTCTAA | 7800 |
| | ATCGGGGGCT CCCTTTAGGG TTCCGATTTA GTGCTTTACG GCACCTCGAC CCCAAAAAAC | 7860 |
| | TTGATTAGGG TGATGGTTCA CGTAGTGGGC CATCGCCCTG ATAGACGGTT TTTCGCCCTT | 7920 |
| | TGACGTTGGA GTCCACGTTC TTTAATAGTG GACTCTTGTT CCAAACTGGA ACAACACTCA | 7980 |
| | ACCCTATCTC GGTCTATTCT TTTGATTTAT AAGGGATTTT GCCGATTTCG GCCTATTGGT | 8040 |
| | TAAAAAATGA GCTGATTTAA CAAAAATTTA ACGCGAATTA ATTCTGTGGA ATGTGTGTCA | 8100 |
| | GTTAGGGTGT GGAAAGTCCC CAGGCTCCCC AGCAGGCAGA AGTATGCAAA GCATGCATCT | 8160 |
| | CAATTAGTCA GCAACCAGGT GTGGAAAGTC CCCAGGCTCC CCAGCAGGCA GAAGTATGCA | 8220 |
| | AAGCATGCAT CTCAATTAGT CAGCAACCAT AGTCCCGCCC CTAACTCCGC CCATCCCGCC | 8280 |
| | CCTAACTCCG CCCAGTTCCG CCCATTCTCC GCCCCATGGC TGACTAATTT TTTTTATTTA | 8340 |
| | TGCAGAGGCC GAGGCCGCCT CTGCCTCTGA GCTATTCCAG AAGTAGTGAG GAGGCTTTTT | 8400 |
| | TGGAGGCCTA GGCT-TTGCA AAAAGCTCCC GGGAGCTTGT ATATCCATTT TCGGATCTGA | 8460 |
| | TCAGCACGTG TTGACAATTA ATCATCGGCA TAGTATATCG GCATAGTATA ATACGACAAG | 8520 |
| | GTGAGGAACT AAACCATGGC CAAGTTGACC AGTGCCGTTC CGGTGCTCAC CGCGCGCGAC | 8580 |
| | GTCGCCGGAG CGGTCGAGTT CTGGACCGAC CGGCTCGGGT TCTCCCGGGA CTTCGTGGAG | 8640 |
| | GACGACTTCG CCGGTGTGGT CCGGGACGAC GTGACCCTGT TCATCAGCGC GGTCCAGGAC | 8700 |
| | CAGGTGGTGC CGGACAACAC CCTGGCCTGG GTGTGGGTGC GCGGCCTGGA CGAGCTGTAC | 8760 |
| | GCCGAGTGGT CGGAGGTCGT GTCCACGAAC TTCCGGGACG CCTCCGGGCC GGCCATGACC | 8820 |
| | GAGATCGGCG AGCAGCCGTG GGGGGGGGAG TTCGCCCTGC GCGACCCGGC CGGCAACTGC | 8880 |
| | GTGCACTTCG TGGCCGAGGA GCAGGACTGA CACGTGCTAC GAGATTTCGA TTCCACCGCC | 8940 |
| | GCCTTCTATG AAAGGTTGGG CTTCGGAATC GTTTTCCGGG ACGCCGGCTG GATGATCCTC | 9000 |
| | CAGCGCGGGG ATCTCATGCT GGAGTTCTTC GCCCACCCCA ACTTGTTTAT TGCAGCTTAT | 9060 |
| | AATGGTTACA AATAAAGCAA TAGCATCACA AATTTCACAA ATAAAGCATT TTTTTCACTG | 9120 |
| | CATTCTAGTT GTGGTTTGTC CAAACTCATC AATGTATCTT ATCATGTCTG TATACCGTCG | 9180 |
| | ACCTCTAGCT AGAGCTTGGC GTAATCATGG TCATAGCTGT TTCCTGTGTG AAATTGTTAT | 9240 |
| | CCGCTCACAA TTCCACACAA CATACGAGCC GGAAGCATAA AGTGTAAAGC CTGGGGTGCC | 9300 |
| | TAATGAGTGA GCTAACTCAC ATTAATTGCG TTGCGCTCAC TGCCCGCTTT CCAGTCGGGA | 9360 |
| | AACCTGTCGT GCCAGCTGCA TTAATGAATC GGCCAACGCG CGGGGAGAGG CGGTTTGCGT | 9420 |
| | ATTGGGCGCT CTTCCGCTTC CTCGCTCACT GACTCGCTGC GCTCGGTCGT TCGGCTGCGG | 9480 |
| | CGAGCGGTAT CAGCTCACTC AAAGGCGGTA ATACGGTTAT CCACAGAATC AGGGGATAAC | 9540 |
| | GCAGGAAAGA ACATGTGAGC AAAAGGCCAG CAAAAGGCCA GGAACCGTAA AAAGGCCGCG | 9600 |
| | TTGCTGGCGT TTTTCCATAG GCTCCGCCCC CCTGACGAGC ATCACAAAAA TCGACGCTCA | 9660 |
| | AGTCAGAGGT GGCGAAACCC GACAGGACTA TAAAGATACC AGGCGTTTCC CCCTGGAAGC | 9720 |
| | TCCCTCGTGC GCTCTCCTGT TCCGACCCTG CCGCTTACCG GATACCTGTC CGCCTTTCTC | 9780 |
| | CCTTCGGGAA GCGTGGCGCT TTCTCATAGC TCACGCTGTA GGTATCTCAG TTCGGTGTAG | 9840 |
| | GTCGTTCGCT CCAAGCTGGG CTGTGTGCAC GAACCCCCCG TTCAGCCCGA CCGCTGCGCC | 9900 |
| | TTATCCGGTA ACTASCGTCT TGAGTCCAAC CCGGTAAGAC ACGACTTATC GCCACTGGCA | 9960 |
| | GCAGCCACTG GTAACAGGAT TAGCAGAGCG AGGTATGTAG GCGGTGCTAC AGAGTTCTTG | 10020 |
| | AAGTGGTGGC CTAACTACGG CTACACTAGA AGAACAGTAT TTGGTATCTG CGCTCTGCTG | 10080 |
| | AAGCCAGTTA CCTTCGGAAA AAGAGTTGGT AGCTCTTGAT CCGGCAAACA AACCACCGCT | 10140 |
| | GGTAGCGGTG GTTTTTTTGT TTGCAAGCAG CAGATTACGC GCAGAAAAAA AGGATCTCAA | 10200 |
| | GAAGATCCTT TGATCTTTTC TACGGGGTCT GACGCTCAGT GGAACGAAAA CTCACGTTAA | 10260 |
| | GGGATTTTGG TCATGAGATT ATCAAAAAGG ATCTTCACCT AGATCCTTTT AAATTAAAAA | 10320 |
| | TGAAGTTTTA AATCAATCTA AAGTATATAT GAGTAAACTT GGTCTGACAG TTACCAATGC | 10380 |
| | TTAATCAGTG AGGCACCTAT CTCAGCGATC TGTCTATTTC GTTCATCCAT AGTTGCCTGA | 10440 |
| | CTCCCCGTCG TGTAGATAAC TACGATACGG GAGGGCTTAC CATCTGGCCC CAGTGCTGCA | 10500 |
| | ATGATACCGC GAGACCCACG CTCACCGGCT CCAGATTTAT CAGCAATAAA CCAGCCAGCC | 10560 |
| | GGAAGGGCCG AGCGCAGAAG TGGTCCTGCA ACTTTATCCG CCTCCATCCA GTCTATTAAT | 10620 |
| | TGTTGCCGGG AAGCTAGAGT AAGTAGTTCG CCAGTTAATA GTTTGCGCAA CGTTGTTGCC | 10680 |
| | ATTGCTACAG GCATCGTGGT GTCACGCTCG TCGTTTGGTA TGGCTTCATT CAGCTCCGGT | 10740 |
| | TCCCAACGAT CAAGGCGAGT TACATGATCC CCCATGTTGT GCAAAAAAGC GGTTAGCTCC | 10800 |
| | TTCGGTCCTC CGATCGTTGT CAGAAGTAAG TTGGCCGCAG TGTTATCACT CATGGTTATG | 10860 |
| | GCAGCACTGC ATAATTCTCT TACTGTCATG CCATCCGTAA GATGCTTTTC TGTGACTGGT | 10920 |
| | GAGTACTCAA CCAAGTCATT CTGAGAATAG TGTATGCGGC GACCGAGTTG CTCTTGCCCG | 10980 |
| | GCGTCAATAC GGGATAATAC CGCGCCACAT AGCAGAACTT TAAAAGTGCT CATCATTGGA | 11040 |
| | AAACGTTCTT CGGGGCGAAA ACTCTCAAGG ATCTTACCGC TGTTGAGATC CAGTTCGATG | 11100 |
| | TAACCCACTC GTGCACCCAA CTGATCTTCA GCATCTTTTA CTTTCACCAG CGTTTCTGGG | 11160 |
| | TGAGCAAAAA CAGGAAGGCA AAATGCCGCA AAAAAGGGAA TAAGGGCGAC ACGGAAATGT | 11220 |
| | TGAATACTCA TACTCTTCCT TTTTCAATAT TATTGAAGCA TTTATCAGGG TTATTGTCTC | 11280 |
| | ATGAGCGGAT ACATATTTGA ATGTATTTAG AAAAATAAAC AAATAGGGGT TCCGCGCACA | 11340 |
| | TTTCCCCGAA AAGTGCCACC TGAC | 11364 |
| SEQ ID NO: 687 | GTCGACGGAT CGGGAGATCT CCCGATCCCC TATGGTGCAC TCTCAGTACA ATCTGCTCTG | 60 |
| CCR11.2: TROP2- | ATGCCGCATA GTTAAGCCAG TATCTGCTCC CTGCTTGTGT GTTGGAGGTC GCTGAGTAGT | 120 |
| IL-2R | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGATTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TGCCTGTGAC AGCGCTGCTG CTGCCACTGG CCCTGCTTCT | 2940 |
| | GCACGCCGCC AGACCCCAGA TCCAGCTGGT GCAGTCAGGC CCTGAACTGA AGAAGCCTGG | 3000 |
| | AGAGACAGTG AAGATTAGCT GCAAGGCCTC GGGATACACC TTCACAAACT ATGGCATGAA | 3060 |
| | CTGGGTCAAA CAGGCCCCTG GCAAGGGCCT TAAATGGATG GGCTGGATCA ACACAAAGAC | 3120 |
| | CGGCGAACCC ACCTACGCCG AGGAGTTCAA GGGCAGATTC GCCTTTTCTC TGGAGACATC | 3180 |
| | CGCCAGCACC GCCTACCTGC AGATCAACAA CCTGAAAAAG GAAGATACAG CCACATACTT | 3240 |
| | CTGCGGACGG GGCGGATACG GTAGCAGCTA CTGGTACTTC GACGTGTGGG GCGCCGGAAC | 3300 |
| | CACAGTGACC GTTTCGTCTG CCAGCACAAA AGGCCCTTCC GGCGGCGGAG GCTCCGGCGG | 3360 |
| | CGGGGGCAGT GGCGGCGGCG GCTCTGGCGG CGGAGGCAGC GACATCGTGA TGACCCAGAG | 3420 |
| | CCACAAGTTC ATGAGCACCA GCGTGGGCGA CCGGGTGTCG ATCACCTGCA AGGCCAGTCA | 3480 |
| | GGATGTGAGC ATCGCCGTCG CCTGGTATCA GCAAAAGCCC GGCCAAAGCC CTAAGGTGCT | 3540 |
| | GATCTATAGC GCCTCCTACA GATACACCGG CGTTCCTGAC AGATTCACAG GGTCTGGCAG | 3600 |
| | CGGCACCGAT TTCACCTTCA CAATCTCCAG AGTGCAGGCC GAGGACCTGG CCGTGTACTA | 3660 |
| | CTGCCAGCAG CACTACATCA CACCTCTGAC CTTTGGTGCT GGCACAAAGC TGGAACTGAA | 3720 |
| | AAGAACCGTG GCCTTCAGAA CTAAGCCTGC TGCCCTCGGC AAGGACACCA TCCCTTGGCT | 3780 |
| | GGGCCACCTG CTGGTGGGCC TGAGCGGAGC TTTTGGCTTC ATCATCCTGG TCTACCTGCT | 3840 |
| | GATTAACTGC AGAAACACCG GTCCTTGGCT GAAGAAAGTG CTGAAGTGCA ACACCCCCGA | 3900 |
| | CCCTAGCAAG TTCTTCAGCC AGCTGAGCAG CGAGCACGGC GGGGACGTGC AAAAATGGCT | 3960 |
| | GAGCAGCCCC TTCCCATCTT CCAGCTTCAG CCCTGGCGGC CTGGCTCCTG AGATCAGCCC | 4020 |
| | CCTGGAAGTG CTGGAACGGG ATAAGGTGAC CCAGCTGTTA CTCCAGCAGG ACAAGGTGCC | 4080 |
| | CGAACCTGCC AGCCTGAGCA GCAACCACAG CCTGACTAGC TGCTTCACCA ACCAGGGCTA | 4140 |
| | CTTCTTCTTT CACCTGCCCG ACGCCCTGGA GATCGAGGCC TGCCAGGTGT ACTTCACCTA | 4200 |
| | CGACCCTTAC AGCGAGGAGG ACCCTGACGA GGGCGTGGCC GGGGCTCCTA CCGGCTCGTC | 4260 |
| | TCCTCAGCCT TTGCAGCCTC TGAGCGGAGA GGATGACGCC TATTGCACCT TTCCTAGCAG | 4320 |
| | AGATGATCTG CTGCTCTTCA GCCCTTCTCT GCTGGGCGGA CCAAGCCCAC CTTCTACCGC | 4380 |
| | ACCTGGCGGC TCTGGCGCAG GCGAAGAGCG GATGCCTCCT TCTCTGCAGG AGAGAGTGCC | 4440 |
| | CCGGGACTGG GACCCTCAGC CGCTGGGACC TCCTACCCCT GGCGTGCCGG ATCTGGTGGA | 4500 |
| | CTTTCAGCCA CCACCTGAGC TGGTGCTGAG AGAAGCCGGC GAAGAGGTGC CTGACGCCGG | 4560 |
| | CCCTAGGGAG GGCGTGAGCT TCCCTTGGAG CCGGCCTCCT GGACAAGGCG AGTTCCGCGC | 4620 |
| | CCTGAACGCC AGACTGCCCC TGAACACCGA CGCCTACCTG AGCCTGCAGG AGCTGCAGGG | 4680 |
| | CCAGGACCCC ACCCACCTGG TGGGCAGCGG AGAGGGAAGA GGCAGCCTGT TGACATGTGG | 4740 |
| | CGATGTCGAG GAAAACCCTG GGCCTATGGC CCTGCCAGTG ACAGCACTAC TCCTGCCTCT | 4800 |
| | GGCCCTGCTG CTCCACGCCG CCAGACCTCA GGTGCAGCTG CAGCAATCTG GCCCCGAGCT | 4860 |
| | AGTGCGGCCT GGCACCAGCG TGCGGATTTC CTGCAAGGCC TCTGGCTACA CCTTCACAAT | 4920 |
| | CTATTGGCTG GGCTGGGTGA AGCAGAGACC TGGACATGGG CTGGAGTGGA TAGGAAACAT | 4980 |
| | CTTCCCTGGC TCAGCTTACA TCAACTACAA CGAGAAGTTT AAGGGAAAAG CCACCCTGAC | 5040 |
| | AGCCGACACC AGCAGCAGCA CCGCCTATAT GCAGCTGTCT AGCCTGACAT CTGAGGATAG | 5100 |
| | CGCCGTTTAC TTCTGCGCCA GAGAAGGCTC CAATTCTGGC TACTGGGGCC AGGGCACAAC | 5160 |
| | CTTAACCGTG TCCAGCGGAG GAGGCGGCTC TGGCGGCGGC GGTTCAGGCG GGGGCGGAAG | 5220 |
| | CGGCGGTGGA GGCTCTGACA TTGTGATGAC CCAGAGCCCC AGCAGCCTGA GCGTGTCTGC | 5280 |
| | TGGCGAGAAG GTGACCATGA CCTGTAAATC TAGCCAGAGC CTGCTGAACA GCGGCAACCA | 5340 |
| | GCAGAACTAC CTGGCCTGGT ATCAGCAGAA ACCTGGCCAG CCCCCCAAGC TGCTGATCTA | 5400 |
| | CGGCGCCAGC ACCAGAGAAA GCGGCGTGCC TGACAGATTC ACTGGCAGCG GCAGCGGCAC | 5460 |
| | AGACTTCACC CTGACCATCA ATAGCGTGCA GGCCGAAGAT CTGGCTGTGT ACTACTGTCA | 5520 |
| | GAGCGACCAC ATCTACCCTT ACACCTTCGG CGGAGGCACC AAGCTAGAAA TCAAGAGCAA | 5580 |
| | GGAAAATCCA TTTCTGTTTG CCCTGGAAGC CGTGGTGATC AGCGTGGGAA GCATGGGCCT | 5640 |
| | GATCATCTCG CTGCTGTGCG TGTACTTCTG GCTGGAAAGG ACCATGCCCC GCATCCCTAC | 5700 |
| | CCTCAAGAAC CTGGAGGACC TGGTCACAGA GTACCACGGC AATTTCAGCG CCTGGTCCGG | 5760 |
| | CGTGTCCAAG GGCCTGGCCG AGAGCCTGCA ACCCGATTAC AGTGAACGGC TGTGTCTGGT | 5820 |
| | GTCTGAGATC CCCCCGAAGG GAGGCGCCCT GGGAGAAGGC CCAGGCGCCA GCCCTTGTAA | 5880 |
| | TCAGCATAGC CCTTACTGGG CTCCTCCATG TTACACCCTG AAGCCCGAAA CCTGAGAATT | 5940 |
| | CGATATCAAG CTTATCGGTA ATCAACCTCT GGATTACAAA ATTTGTGAAA GATTGACTGG | 6000 |
| | TATTCTTAAC TATGTTGCTC CTTTTACGCT ATGTGGATAC GCTGCTTTAA TGCCTTTGTA | 6060 |
| | TCATGCTATT GCTTCCCGTA TGGCTTTCAT TTTCTCCTCC TTGTATAAAT CCTGGTTGCT | 6120 |
| | GTCTCTTTAT GAGGAGTTGT GGCCCGTTGT CAGGCAACGT GGCGTGGTGT GCACTGTGTT | 6180 |
| | TGCTGACGCA ACCCCCACTG GTTGGGGCAT TGCCACCACC TGTCAGCTCC TTTCCGGGAC | 6240 |
| | TTTCGCTTTC CCCC CCCTA TTGCCACGGC GGAACTCATC GCCGCCTGCC TTGCCCGCTG | 6300 |
| | CTGGACAGGG GCTCGGCTGT TGGGCACTGA CAATTCCGTG GTGTTGTCGG GGAAATCATC | 6360 |
| | GTCCTTTCCT TGGCTGCTCG CCTGTGTTGC CACCTGGATT CTGCGCGGGA CGTCCTTCTG | 6420 |
| | CTACGTCCCT TCGGCCCTCA ATCCAGCGGA CCTTCCTTCC CGCGGCCTGC TGCCGGCTCT | 6480 |
| | GCGGCCTCTT CCGCGTCTTC GCCTTCGCCC TCAGACGAGT CGGATCTCCC TTTGGGCCGC | 6540 |
| | CTCCCCGCAT CGATACCGTC GACCTCGAGA CCTAGAAAAA CATGGAGCAA TCACAAGTAG | 6600 |
| | CAATACAGCA GCTACCAATG CTGATTGTGC CTGGCTAGAA GCACAAGAGG AGGAGGAGGT | 6660 |
| | GGGTTTTCCA GTCACACCTC AGGTACCTTT AAGACCAATG ACTTACAAGG CAGCTGTAGA | 6720 |
| | TCTTAGCCAC TTTTTAAAAG AAAAGGGGGG ACTGGAAGGG CTAATTCACT CCCAACGAAG | 6780 |
| | ACAAGATATC CTTGATCTGT GGATCTACCA CACACAAGGC TACTTCCCTG ATTGGCAGAA | 6840 |
| | CTACACACCA GGGCCAGGGA TCAGATATCC ACTGACCTTT GGATGGTGCT ACAAGCTAGT | 6900 |
| | ACCAGTTGAG CAAGAGAAGG TAGAAGAAGC CAATGAAGGA GAGAACACCC GCTTGTTACA | 6960 |
| | CCCTGTGAGC CTGCATGGGA TGGATGACCC GGAGAGAGAA GTATTAGAGT GGAGGTTTGA | 7020 |
| | CAGCCGCCTA GCATTTCATC ACATGGCCCG AGAGCTGCAT CCGGACTGTA CTGGGTCTCT | 7080 |
| | CTGGTTAGAC CAGASCTGAG CCTGGGAGCT CTCTGGCTAA CTAGGGAACC CACTGCTTAA | 7140 |
| | GCCTCAATAA AGCTTGCCTT GAGTGCTTCA AGTAGTGTGT GCCCGTCTGT TGTGTGACTC | 7200 |
| | TGGTAACTAG AGATCCCTCA GACCCTTTTA GTCAGTGTGG AAAATCTCTA GCAGGGCCCG | 7260 |
| | TTTAAACCCG CTGASCAGCC TCGACTGTGC CTTCTAGTTG CCAGCCATCT GTTGTTTGCC | 7320 |
| | CCTCCCCCGT GCCTTCCTTG ACCCTGGAAG GTGCCACTCC CACTGTCCTT TCCTAATAAA | 7380 |
| | ATGAGGAAAT TGCATCGCAT TGTCTGAGTA GGTGTCATTC TATTCTGGGG GGTGGGGTGG | 7440 |
| | GGCAGGACAG CAAGGGGGAG GATTGGGAAG ACAATAGCAG GCATGCTGGG GATGCGGTGG | 7500 |
| | GCTCTATGGC TTCTGAGGCG GAAAGAACCA GCTGGGGCTC TAGGGGGTAT CCCCACGCGC | 7560 |
| | CCTGTAGCGG CGCASTAAGC GCGGCGGGTG TGGTGGTTAC GCGCAGCGTG ACCGCTACAC | 7620 |
| | TTGCCAGCGC CCTAGCGCCC GCTCCTTTCG CTTTCTTCCC TTCCTTTCTC GCCACGTTCG | 7680 |
| | CCGGCTTTCC CCGTCAAGCT CTAAATCGGG GGCTCCCTTT AGGGTTCCGA TTTAGTGCTT | 7740 |
| | TACGGCACCT CGACCCCAAA AAACTTGATT AGGGTGATGG TTCACGTAGT GGGCCATCGC | 7800 |
| | CCTGATAGAC GGTTTTTCGC CCTTTGACGT TGGAGTCCAC GTTCTTTAAT AGTGGACTCT | 7860 |
| | TGTTCCAAAC TGGAACAACA CTCAACCCTA TCTCGGTCTA TTCTTTTGAT TTATAAGGGA | 7920 |
| | TTTTGCCGAT TTCGGCCTAT TGGTTAAAAA ATGAGCTGAT TTAACAAAAA TTTAACGCGA | 7980 |
| | ATTAATTCTG TGGAATGTGT GTCAGTTAGG GTGTGGAAAG TCCCCAGGCT CCCCAGCAGG | 8040 |
| | CAGAAGTATG CAAAGCATGC ATCTCAATTA GTCAGCAACC AGGTGTGGAA AGTCCCCAGG | 8100 |
| | CTCCCCAGCA GGCAGAAGTA TGCAAAGCAT GCATCTCAAT TAGTCAGCAA CCATAGTCCC | 8160 |
| | GCCCCTAACT CCGCCCATCC CGCCCCTAAC TCCGCCCAGT TCCGCCCATT CTCCGCCCCA | 8220 |
| | TGGCTGACTA ATTTTTTTTA TTTATGCAGA GGCCGAGGCC GCCTCTGCCT CTGAGCTATT | 8280 |
| | CCAGAAGTAG TGAGGAGGCT TTTTTGGAGG CCTAGGCTTT TGCAAAAAGC TCCCGGGAGC | 8340 |
| | TTGTATATCC ATTTTCGGAT CTGATCAGCA CGTGTTGACA ATTAATCATC GGCATAGTAT | 8400 |
| | ATCGGCATAG TATAATACGA CAAGGTGAGG AACTAAACCA TGGCCAAGTT GACCAGTGCC | 8460 |
| | GTTCCGGTGC TCACCGCGCG CGACGTCGCC GGAGCGGTCG AGTTCTGGAC CGACCGGCTC | 8520 |
| | GGGTTCTCCC GGGACTTCGT GGAGGACGAC TTCGCCGGTG TGGTCCGGGA CGACGTGACC | 8580 |
| | CTGTTCATCA GCGCGGTCCA GGACCAGGTG GTGCCGGACA ACACCCTGGC CTGGGTGTGG | 8640 |
| | GTGCGCGGCC TGGACGAGCT GTACGCCGAG TGGTCGGAGG TCGTGTCCAC GAACTTCCGG | 8700 |
| | GACGCCTCCG GGCCGGCCAT GACCGAGATC GGCGAGCAGC CGTGGGGGCG GGAGTTCGCC | 8760 |
| | CTGCGCGACC CGGCCGGCAA CTGCGTGCAC TTCGTGGCCG AGGAGCAGGA CTGACACGTG | 8820 |
| | CTACGAGATT TCGATTCCAC CGCCGCCTTC TATGAAAGGT TGGGCTTCGG AATCGTTTTC | 8880 |
| | CGGGACGCCG GCTGGATGAT CCTCCAGCGC GGGGATCTCA TGCTGGAGTT CTTCGCCCAC | 8940 |
| | CCCAACTTGT TTATTGCAGC TTATAATGGT TACAAATAAA GCAATAGCAT CACAAATTTC | 9000 |
| | ACAAATAAAG CATTTTTTTC ACTGCATTCT AGTIGTGGTT TGTCCAAACT CATCAATGTA | 9060 |
| | TCTTATCATG TCTGTATACC GTCGACCTCT AGCTAGAGCT TGGCGTAATC ATGGTCATAG | 9120 |
| | CTGTTTCCTG TGTGAAATTG TTATCCGCTC ACAATTCCAC ACAACATACG AGCCGGAAGC | 9180 |
| | ATAAAGTGTA AAGCCTGGGG TGCCTAATGA GTGAGCTAAC TCACATTAAT TGCGTTGCGC | 9240 |
| | TCACTGCCCG CTTTCCAGTC GGGAAACCTG TCGTGCCAGC TGCATTAATG AATCGGCCAA | 9300 |
| | CGCGCGGGGA GAGGCGGTTT GCGTATTGGG CGCTCTTCCG CTTCCTCGCT CACTGACTCG | 9360 |
| | CTGCGCTCGG TCGTTCGGCT GCGGCGAGCG GTATCAGCTC ACTCAAAGGC GGTAATACGG | 9420 |
| | TTATCCACAG AATCAGGGGA TAACGCAGGA AAGAACATGT GAGCAAAAGG CCAGCAAAAG | 9480 |
| | GCCAGGAACC GTAAAAAGGC CGCGTTGCTG GCGTTTTTCC ATAGGCTCCG CCCCCCTGAC | 9540 |
| | GAGCATCACA AAAATCGACG CTCAAGTCAG AGGTGGCGAA ACCCGACAGG ACTATAAAGA | 9600 |
| | TACCAGGCGT TTCCCCCTGG AAGCTCCCTC GTGCGCTCTC CTGTTCCGAC CCTGCCGCTT | 9660 |
| | ACCGGATACC TGTCCGCCTT TCTCCCTTCG GGAAGCGTGG CGCTTTCTCA TAGCTCACGC | 9720 |
| | TGTAGGTATC TCAGTTCGGT GTAGGTCGTT CGCTCCAAGC TGGGCTGTGT GCACGAACCC | 9780 |
| | CCCGTTCAGC CCGACCGCTG CGCCTTATCC GGTAACTATC GTCTTGAGTC CAACCCGGTA | 9840 |
| | AGACACGACT TATCGCCACT GGCAGCAGCC ACTGGTAACA GGATTAGCAG AGCGAGGTAT | 9900 |
| | GTAGGCGGTG CTACAGAGTT CTTGAAGTGG TGGCCTAACT ACGGCTACAC TAGAAGAACA | 9960 |
| | GTATTTGGTA TCTGCGCTCT GCTGAAGCCA GTTACCTTCG GAAAAAGAGT TGGTAGCTCT | 10020 |
| | TGATCCGGCA AACAAACCAC CGCTGGTAGC GGTGGTTTTT TTGTTTGCAA GCAGCAGATT | 10080 |
| | ACGCGCAGAA AAAAAGGATC TCAAGAAGAT CCTTTGATCT TTTCTACGGG GTCTGACGCT | 10140 |
| | CAGTGGAACG AAAACTCACG TTAAGGGATT TTGGTCATGA GATTATCAAA AAGGATCTTC | 10200 |
| | ACCTAGATCC TTTTAAATTA AAAATGAAGT TTTAAATCAA TCTAAAGTAT ATATGAGTAA | 10260 |
| | ACTTGGTCTG ACAGTTACCA ATGCTTAATC AGTGAGGCAC CTATCTCAGC GATCTGTCTA | 10320 |
| | TTTCGTTCAT CCATAGTTGC CTGACTCCCC GTCGTGTAGA TAACTACGAT ACGGGAGGGC | 10380 |
| | TTACCATCTG GCCCCAGTGC TGCAATGATA CCGCGAGACC CACGCTCACC GGCTCCAGAT | 10440 |
| | TTATCAGCAA TAAACCAGCC AGCCGGAAGG GCCGAGCGCA GAAGTGGTCC TGCAACTTTA | 10500 |
| | TCCGCCTCCA TCCAGTCTAT TAATTGTTGC CGGGAAGCTA GAGTAAGTAG TTCGCCAGTT | 10560 |
| | AATAGTTTGC GCAACGTTGT TGCCATTGCT ACAGGCATCG TGGTGTCACG CTCGTCGTTT | 10620 |
| | GGTATGGCTT CATTCAGCTC CGGTTCCCAA CGATCAAGGC GAGTTACATG ATCCCCCATG | 10680 |
| | TTGTGCAAAA AAGCGGTTAG CTCCTTCGGT CCTCCGATCG TTGTCAGAAG TAAGTTGGCC | 10740 |
| | GCAGTGTTAT CACTCATGGT TATGGCAGCA CTGCATAATT CTCTTACTGT CATGCCATCC | 10800 |
| | GTAAGATGCT TTTCTGTGAC TGGTGAGTAC TCAACCAAGT CATTCTGAGA ATAGTGTATG | 10860 |
| | CGGCGACCGA GTTGCTCTTG CCCGGCGTCA ATACGGGATA ATACCGCGCC ACATAGCAGA | 10920 |
| | ACTTTAAAAG TGCTCATCAT TGGAAAACGT TCTTCGGGGC GAAAACTCTC AAGGATCTTA | 10980 |
| | CCGCTGTTGA GATCCAGTTC GATGTAACCC ACTCGTGCAC CCAACTGATC TTCAGCATCT | 11040 |
| | TTTACTTTCA CCAGCGTTTC TGGGTGAGCA AAAACAGGAA GGCAAAATGC CGCAAAAAAG | 11100 |
| | GGAATAAGGG CGACACGGAA ATGTTGAATA CTCATACTCT TCCTTTTTCA ATATTATTGA | 11160 |
| | AGCATTTATC AGGGSTATTG TCTCATGAGC GGATACATAT TTGAATGTAT TTAGAAAAAT | 11220 |
| | AAACAAATAG GGGTTCCGCG CACATTTCCC CGAAAAGTGC CACCTGAC | 11268 |
| SEQ ID NO: 688 | GTCGACGGAT CGGGAGATCT CCCGATCCCC TATGGTGCAC TCTCAGTACA ATCTGCTCTG | 60 |
| CCR12.2: TROP2- | ATGCCGCATA GTTAAGCCAG TATCTGCTCC CTGCTTGTGT GTTGGAGGTC GCTGAGTAGT | 120 |
| IL-18R | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGATTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGCCC TGCCTGTGAC CGCGCTGCTG CTGCCTCTGG CCCTGCTGCT | 2940 |
| | GCATGCCGCC AGGCCACAGA TCCAGCTGGT GCAGAGCGGA CCTGAACTGA AGAAGCCTGG | 3000 |
| | GGAAACCGTG AAGATCAGCT GCAAGGCCTC CGGCTACACC TTCACAAACT ACGGCATGAA | 3060 |
| | CTGGGTGAAG CAGGCCCCTG GAAAGGGCCT GAAATGGATG GGCTGGATCA ACACCAAGAC | 3120 |
| | CGGCGAGCCT ACCTACGCCG AAGAGTTCAA GGGCAGATTC GCCTTCAGCC TGGAGACCTC | 3180 |
| | AGCCAGCACC GCCTACCTGC AGATCAACAA CCTGAAGAAG GAAGATACCG CCACCTACTT | 3240 |
| | CTGCGGCCGG GGCGGTTATG GCAGCAGCTA CTGGTACTTC GACGTGTGGG GCGCCGGCAC | 3300 |
| | AACAGTGACA GTATCCAGCG CCTCCACAAA AGGACCTAGC GGCGGCGGCG GCAGCGGTGG | 3360 |
| | TGGCGGAAGT GGCGGCGGCG GCAGCGGCGG CGGTGGAAGC GACATCGTGA TGACCCAGAG | 3420 |
| | CCATAAGTTC ATGAGCACAA GCGTGGGCGA TCGGGTGAGC ATCACCTGCA AGGCCTCTCA | 3480 |
| | GGACGTGAGC ATCGCCGTGG CCTGGTATCA GCAGAAACCT GGGCAGTCCC CTAAGGTGTT | 3540 |
| | GATCTATTCT GCTTCCTACC GATACACAGG AGTGCCTGAC CGGTTCACCG GCTCTGGCAG | 3600 |
| | CGGCACCGAT TTCACCTTTA CAATCAGCAG AGTGCAGGCT GAGGACCTGG CCGTGTACTA | 3660 |
| | CTGTCAGCAG CACTACATCA CCCCTCTGAC CTTTGGCGCC GGAACTAAAC TGGAACTGAA | 3720 |
| | GCGGACCGTG GCCGATATGG CTGATATACC CGGCCACGTG TTCACTAGAG GCATGATCAT | 3780 |
| | TGCCGTGTTG ATCCTGGTGG CCGTGGTTTG CCTGGTGACC GTGTGCGTGA TCTACAGAGT | 3840 |
| | GGACCTGGTC CTGTTCTACA GACACCTGAC CAGACGCGAC GAGACCCTGA CAGACGGCAA | 3900 |
| | AACATACGAC GCCTTCGTGT CCTACCTGAA AGAGTGCAGA CCTGAGAATG GCGAGGAACA | 3960 |
| | CACCTTTGCC GTCGAGATCC TGCCCAGAGT GCTGGAAAAG CACTTCGGCT ATAAGCTGTG | 4020 |
| | TATCTTCGAG CGGGACGTGG TGCCAGGCGG CGCCGTGGTG GACGAGATCC ACAGCCTGAT | 4080 |
| | TGAGAAAAGC CGGCGGCTGA TCATCGTGCT GAGCAAGTCG TACATGAGCA ATGAAGTGCG | 4140 |
| | GTACGAGCTG GAAAGCGGCC TGCACGAGGC CCTGGTCGAG AGAAAGATTA AGATCATCCT | 4200 |
| | GATCGAGTTT ACACCCGTGA CAGACTTTAC CTTCCTGCCC CAGAGCCTGA AACTGCTGAA | 4260 |
| | GAGCCATAGA GTGCTGAAGT GGAAGGCTGA TAAGTCTCTG AGCTACAACT CTCGCTTCTG | 4320 |
| | GAAGAACCTG CTCTACCTGA TGCCTGCCAA GACAGTTAAG CCCGGCAGAG ACGAGCCCGA | 4380 |
| | GGTGCTGCCT GTGCTTTCCG AGAGCGGATC TGGCGAAGGC AGAGGTTCCC TGCTTACATG | 4440 |
| | TGGCGACGTG GAGGAAAATC CTGGCCCTAT GGCCCTGCCT GTTACCGCTC TGCTGCTGCC | 4500 |
| | TCTGGCCCTG CTGCTGCACG CCGCTAGACC TCAGGTGCAA CTGCAACAGA GCGGCCCTGA | 4560 |
| | ACTGGTCAGA CCCGGAACCT CCGTGCGGAT CAGTTGCAAG GCCAGCGGCT ACACCTTCAC | 4620 |
| | CATCTACTGG CTGGGATGGG TTAAGCAGAG ACCTGGCCAC GGCTTGGAGT GGATCGGCAA | 4680 |
| | CATCTTTCCC GGCTCCGCCT ATATCAACTA CAACGAGAAA TTCAAAGGGA AGGCGACCCT | 4740 |
| | GACAGCCGAC ACCAGCAGTT CCACCGCCTA CATGCAGCTG TCCAGCCTGA CATCTGAGGA | 4800 |
| | CAGCGCAGTG TATT-TTGCG CCAGAGAAGG CAGCAACAGC GGATACTGGG GACAGGGCAC | 4860 |
| | CACACTCACC GTGAGCAGCG GAGGCGGCGG CAGCGGCGGC GGCGGAAGCG GCGGCGGCGG | 4920 |
| | GAGCGGCGGC GGCGGATCTG ACATCGTGAT GACCCAAAGC CCTAGCAGCC TGAGTGTGAG | 4980 |
| | CGCCGGCGAG AAGGTGACCA TGACCTGTAA AAGCAGCCAG TCGCTGCTGA ACAGCGGCAA | 5040 |
| | TCAGCAGAAC TACCTGGCAT GGTATCAGCA AAAGCCCGGC CAGCCTCCAA AGCTGCTTAT | 5100 |
| | CTACGGTGCC AGCACCCGGG AGAGCGGCGT TCCTGATCGG TTCACTGGAA GTGGCAGCGG | 5160 |
| | AACCGACTTC ACACTGACGA TCAATAGCGT GCAGGCCGAG GACCTGGCCG TGTACTACTG | 5220 |
| | CCAAAGCGAC CACASCTACC CCTACACCTT CGGGGGGGGC ACCAAGCTCG AAATCAAGAA | 5280 |
| | CACAACACAG TCTGTGCAGC TGAAAGAGAA GAGAGGCGTG GTTCTGCTGT ACATCCTTCT | 5340 |
| | TGGAACCATC GGCACCCTGG TGGCCGTGTT AGCCGCCTCT GCTCTGCTGT ACCGGCACTG | 5400 |
| | GATCGAGATC GTGCTGCTGT ATAGAACATA CCAGTCTAAG GACCAGACCC TGGGCGACAA | 5460 |
| | GAAAGATTTC GATGCCTTCG TGTCCTACGC TAAGTGGAGC AGCTTCCCTT CTGAAGCCAC | 5520 |
| | CTCTAGCCTG TCTGAAGAGC ACCTGGCCCT GAGCCTGTTT CCAGATGTGC TGGAGAACAA | 5580 |
| | GTACGGATAC AGCCTGTGCC TGCTGGAAAG AGACGTGGCC CCTGGAGGCG TGTACGCCGA | 5640 |
| | AGACATCGTC AGCATCATCA AGCGGAGCAG AAGAGGCATC TTCATCCTGA GCCCAAACTA | 5700 |
| | CGTGAACGGC CCCAGCATCT TCGAGCTGCA GGCCGCCGTG AACCTGGCTC TTGATGACCA | 5760 |
| | AACACTGAAG CTGATCCTGA TCAAGTTCTG CTACTTTCAG GAGCCTGAAT CCCTGCCGCA | 5820 |
| | CCTGGTGAAG AAGGCCCTGA GAGTTCTGCC CACCGTGACC TGGCGCGGAC TGAAATCTGT | 5880 |
| | TCCTCCTAAT AGCAGGTTCT GGGCCAAGAT GAGATACCAC ATGCCAGTGA AGAACAGCCA | 5940 |
| | GGGGTTCACC TGGAACCAGC TGAGAATCAC AAGCAGAATC TTCCAGTGGA AGGGCCTGTC | 6000 |
| | TCGAACAGAG ACCACCGGCA GAAGCAGCCA ACCTAAGGAA TGGTGAGAAT TCGATATCAA | 6060 |
| | GCTTATCGGT AATCAACCTC TGGATTACAA AATTTGTGAA AGATTGACTG GTATTCTTAA | 6120 |
| | CTATGTTGCT CCTTTTACGC TATGTGGATA CGCTGCTTTA ATGCCTTTGT ATCATGCTAT | 6180 |
| | TGCTTCCCGT ATGGCTTTCA TTTTCTCCTC CTTGTATAAA TCCTGGTTGC TGTCTCTTTA | 6240 |
| | TGAGGAGTTG TGGCCCGTTG TCAGGCAACG TGGCGTGGTG TGCACTGTGT TTGCTGACGC | 6300 |
| | AACCCCCACT GGTTGGGGCA TTGCCACCAC CTGTCAGCTC CTTTCCGGGA CTTTCGCTTT | 6360 |
| | CCCCCTCCCT ATTGCCACGG CGGAACTCAT CGCCGCCTGC CTTGCCCGCT GCTGGACAGG | 6420 |
| | GGCTCGGCTG TTGGGCACTG ACAATTCCGT GGTGTTGTCG GGGAAATCAT CGTCCTTTCC | 6480 |
| | TTGGCTGCTC GCCTGTGTTG CCACCTGGAT TCTGCGCGGG ACGTCCTTCT GCTACGTCCC | 6540 |
| | TTCGGCCCTC AATCCAGCGG ACCTTCCTTC CCGCGGCCTG CTGCCGGCTC TGCGGCCTCT | 6600 |
| | TCCGCGTCTT CGCCTTCGCC CTCAGACGAG TCGGATCTCC CTTTGGGCCG CCTCCCCGCA | 6660 |
| | TCGATACCGT CGACCTCGAG ACCTAGAAAA ACATGGAGCA ATCACAAGTA GCAATACAGC | 6720 |
| | AGCTACCAAT GCTGATTGTG CCTGGCTAGA AGCACAAGAG GAGGAGGAGG TGGGTTTTCC | 6780 |
| | AGTCACACCT CAGGTACCTT TAAGACCAAT GACTTACAAG GCAGCTGTAG ATCTTAGCCA | 6840 |
| | CTTTTTAAAA GAAAAGGGGG GACTGGAAGG GCTAATTCAC TCCCAACGAA GACAAGATAT | 6900 |
| | CCTTGATCTG TGGATCTACC ACACACAAGG CTACTTCCCT GATTGGCAGA ACTACACACC | 6960 |
| | AGGGCCAGGG ATCAGATATC CACTGACCTT TGGATGGTGC TACAAGCTAG TACCAGTTGA | 7020 |
| | GCAAGAGAAG GTAGAAGAAG CCAATGAAGG AGAGAACACC CGCTTGTTAC ACCCTGTGAG | 7080 |
| | CCTGCATGGG ATGGATGACC CGGAGAGAGA AGTATTAGAG TGGAGGTTTG ACAGCCGCCT | 7140 |
| | AGCATTTCAT CACATGGCCC GAGAGCTGCA TCCGGACTGT ACTGGGTCTC TCTGGTTAGA | 7200 |
| | CCAGATCTGA GCCTGGGAGC TCTCTGGCTA ACTAGGGAAC CCACTGCTTA AGCCTCAATA | 7260 |
| | AAGCTTGCCT TGAGTGCTTC AAGTAGTGTG TGCCCGTCTG TTGTGTGACT CTGGTAACTA | 7320 |
| | GAGATCCCTC AGACCCTTTT AGTCAGTGTG GAAAATCTCT AGCAGGGCCC GTTTAAACCC | 7380 |
| | GCTGATCAGC CTCGACTGTG CCTTCTAGTT GCCAGCCATC TGTTGTTTGC CCCTCCCCCG | 7440 |
| | TGCCTTCCTT GACCCTGGAA GGTGCCACTC CCACTGTCCT TTCCTAATAA AATGAGGAAA | 7500 |
| | TTGCATCGCA TTGTCTGAGT AGGTGTCATT CTATTCTGGG GGGTGGGGTG GGGCAGGACA | 7560 |
| | GCAAGGGGGA GGATTGGGAA GACAATAGCA GGCATGCTGG GGATGCGGTG GGCTCTATGG | 7620 |
| | CTTCTGAGGC GGAAAGAACC AGCTGGGGCT CTAGGGGGTA TCCCCACGCG CCCTGTAGCG | 7680 |
| | GCGCATTAAG CGCGGCGGGT GTGGTGGTTA CGCGCAGCGT GACCGCTACA CTTGCCAGCG | 7740 |
| | CCCTAGCGCC CGCTCCTTTC GCTTTCTTCC CTTCCTTTCT CGCCACGTTC GCCGGCTTTC | 7800 |
| | CCCGTCAAGC TCTAAATCGG GGGCTCCCTT TAGGGTTCCG ATTTAGTGCT TTACGGCACC | 7860 |
| | TCGACCCCAA AAAACTTGAT TAGGGTGATG GTTCACGTAG TGGGCCATCG CCCTGATAGA | 7920 |
| | CGGTTTTTCG CCCTTTGACG TTGGAGTCCA CGTTCTTTAA TAGTGGACTC TTGTTCCAAA | 7980 |
| | CTGGAACAAC ACTCAACCCT ATCTCGGTCT ATTCTTTTGA TTTATAAGGG ATTTTGCCGA | 8040 |
| | TTTCGGCCTA TTGGTTAAAA AATGAGCTGA TTTAACAAAA ATTTAACGCG AATTAATTCT | 8100 |
| | GTGGAATGTG TGTCAGTTAG GGTGTGGAAA GTCCCCAGGC TCCCCAGCAG GCAGAAGTAT | 8160 |
| | GCAAAGCATG CATCTCAATT AGTCAGCAAC CAGGTGTGGA AAGTCCCCAG GCTCCCCAGC | 8220 |
| | AGGCAGAAGT ATGCAAAGCA TGCATCTCAA TTAGTCAGCA ACCATAGTCC CGCCCCTAAC | 8280 |
| | TCCGCCCATC CCGCCCCTAA CTCCGCCCAG TTCCGCCCAT TCTCCGCCCC ATGGCTGACT | 8340 |
| | AATTTTTTTT ATTTATGCAG AGGCCGAGGC CGCCTCTGCC TCTGAGCTAT TCCAGAAGTA | 8400 |
| | GTGAGGAGGC TTTTTTGGAG GCCTAGGCTT TTGCAAAAAG CTCCCGGGAG CTTGTATATC | 8460 |
| | CATTTTCGGA TCTGATCAGC ACGTGTTGAC AATTAATCAT CGGCATAGTA TATCGGCATA | 8520 |
| | GTATAATACG ACAAGGTGAG GAACTAAACC ATGGCCAAGT TGACCAGTGC CGTTCCGGTG | 8580 |
| | CTCACCGCGC GCGACGTCGC CGGAGCGGTC GAGTTCTGGA CCGACCGGCT CGGGTTCTCC | 8640 |
| | CGGGACTTCG TGGAGGACGA CTTCGCCGGT GTGGTCCGGG ACGACGTGAC CCTGTTCATC | 8700 |
| | AGCGCGGTCC AGGACCAGGT GGTGCCGGAC AACACCCTGG CCTGGGTGTG GGTGCGCGGC | 8760 |
| | CTGGACGAGC TGTACGCCGA GTGGTCGGAG GTCGTGTCCA CGAACTTCCG GGACGCCTCC | 8820 |
| | GGGCCGGCCA TGACCGAGAT CGGCGAGCAG CCGTGGGGGC GGGAGTTCGC CCTGCGCGAC | 8880 |
| | CCGGCCGGCA ACTGCGTGCA CTTCGTGGCC GAGGAGCAGG ACTGACACGT GCTACGAGAT | 8940 |
| | TTCGATTCCA CCGCCGCCTT CTATGAAAGG TTGGGCTTCG GAATCGTTTT CCGGGACGCC | 9000 |
| | GGCTGGATGA TCCTCCAGCG CGGGGATCTC ATGCTGGAGT TCTTCGCCCA CCCCAACTTG | 9060 |
| | TTTATTGCAG CTTATAATGG TTACAAATAA AGCAATAGCA TCACAAATTT CACAAATAAA | 9120 |
| | GCATTTTTTT CACTGCATTC TAGTTGTGGT TTGTCCAAAC TCATCAATGT ATCTTATCAT | 9180 |
| | GTCTGTATAC CGTCGACCTC TAGCTAGAGC TTGGCGTAAT CATGGTCATA GCTGTTTCCT | 9240 |
| | GTGTGAAATT GTTATCCGCT CACAATTCCA CACAACATAC GAGCCGGAAG CATAAAGTGT | 9300 |
| | AAAGCCTGGG GTGCCTAATG AGTGAGCTAA CTCACATTAA TTGCGTTGCG CTCACTGCCC | 9360 |
| | GCTTTCCAGT CGGGAAACCT GTCGTGCCAG CTGCATTAAT GAATCGGCCA ACGCGCGGGG | 9420 |
| | AGAGGCGGTT TGCGTATTGG GCGCTCTTCC GCTTCCTCGC TCACTGACTC GCTGCGCTCG | 9480 |
| | GTCGTTCGGC TGCGGCGAGC GGTATCAGCT CACTCAAAGG CGGTAATACG GTTATCCACA | 9540 |
| | GAATCAGGGG ATAACGCAGG AAAGAACATG TGAGCAAAAG GCCAGCAAAA GGCCAGGAAC | 9600 |
| | CGTAAAAAGG CCGCGTTGCT GGCGTTTTTC CATAGGCTCC GCCCCCCTGA CGAGCATCAC | 9660 |
| | AAAAATCGAC GCTCAAGTCA GAGGTGGCGA AACCCGACAG GACTATAAAG ATACCAGGCG | 9720 |
| | TTTCCCCCTG GAAGCTCCCT CGTGCGCTCT CCTGTTCCGA CCCTGCCGCT TACCGGATAC | 9780 |
| | CTGTCCGCCT TTCTCCCTTC GGGAAGCGTG GCGCTTTCTC ATAGCTCACG CTGTAGGTAT | 9840 |
| | CTCAGTTCGG TGTAGGTCGT TCGCTCCAAG CTGGGCTGTG TGCACGAACC CCCCGTTCAG | 9900 |
| | CCCGACCGCT GCGCCTTATC CGGTAACTAT CGTCTTGAGT CCAACCCGGT AAGACACGAC | 9960 |
| | TTATCGCCAC TGGCAGCAGC CACTGGTAAC AGGATTAGCA GAGCGAGGTA TGTAGGCGGT | 10020 |
| | GCTACAGAGT TCTTGAAGTG GTGGCCTAAC TACGGCTACA CTAGAAGAAC AGTATTTGGT | 10080 |
| | ATCTGCGCTC TGCTGAAGCC AGTTACCTTC GGAAAAAGAG TTGGTAGCTC TTGATCCGGC | 10140 |
| | AAACAAACCA CCGCIGGTAG CGGTGGTTTT TTTGTTTGCA AGCAGCAGAT TACGCGCAGA | 10200 |
| | AAAAAAGGAT CTCAAGAAGA TCCTTTGATC TTTTCTACGG GGTCTGACGC TCAGTGGAAC | 10260 |
| | GAAAACTCAC GTTAAGGGAT TTTGGTCATG AGATTATCAA AAAGGATCTT CACCTAGATC | 10320 |
| | CTTTTAAATT AAAAATGAAG TTTTAAATCA ATCTAAAGTA TATATGAGTA AACTTGGTCT | 10380 |
| | GACAGTTACC AATGCTTAAT CAGTGAGGCA CCTATCTCAG CGATCTGTCT ATTTCGTTCA | 10440 |
| | TCCATAGTTG CCTGACTCCC CGTCGTGTAG ATAACTACGA TACGGGAGGG CTTACCATCT | 10500 |
| | GGCCCCAGTG CTGCAATGAT ACCGCGAGAC CCACGCTCAC CGGCTCCAGA TTTATCAGCA | 10560 |
| | ATAAACCAGC CAGCCGGAAG GGCCGAGCGC AGAAGTGGTC CTGCAACTTT ATCCGCCTCC | 10620 |
| | ATCCAGTCTA TTAATTGTTG CCGGGAAGCT AGAGTAAGTA GTTCGCCAGT TAATAGTTTG | 10680 |
| | CGCAACGTTG TTGCCATTGC TACAGGCATC GTGGTGTCAC GCTCGTCGTT TGGTATGGCT | 10740 |
| | TCATTCAGCT CCGGCTCCCA ACGATCAAGG CGAGTTACAT GATCCCCCAT GTTGTGCAAA | 10800 |
| | AAAGCGGTTA GCTCCTTCGG TCCTCCGATC GTTGTCAGAA GTAAGTTGGC CGCAGTGTTA | 10860 |
| | TCACTCATGG TTATGGCAGC ACTGCATAAT TCTCTTACTG TCATGCCATC CGTAAGATGC | 10920 |
| | TTTTCTGTGA CTGGTGAGTA CTCAACCAAG TCATTCTGAG AATAGTGTAT GCGGCGACCG | 10980 |
| | AGTTGCTCTT GCCCGGCGTC AATACGGGAT AATACCGCGC CACATAGCAG AACTTTAAAA | 11040 |
| | GTGCTCATCA TTGGAAAACG TTCTTCGGGG CGAAAACTCT CAAGGATCTT ACCGCTGTTG | 11100 |
| | AGATCCAGTT CGATGTAACC CACTCGTGCA CCCAACTGAT CTTCAGCATC TTTTACTTTC | 11160 |
| | ACCAGCGTTT CTGGGTGAGC AAAAACAGGA AGGCAAAATG CCGCAAAAAA GGGAATAAGG | 11220 |
| | GCGACACGGA AATGTTGAAT ACTCATACTC TTCCTTTTTC AATATTATTG AAGCATTTAT | 11280 |
| | CAGGGTTATT GTCTCATGAG CGGATACATA TTTGAATGTA TTTAGAAAAA TAAACAAATA | 11340 |
| | GGGGTTCCGC GCACATTTCC CCGAAAAGTG CCACCTGAC | 11379 |

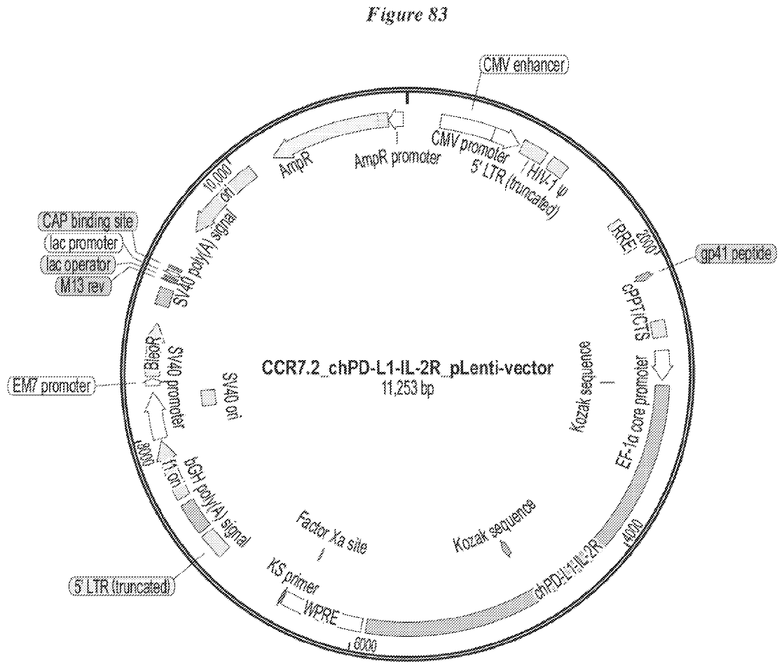

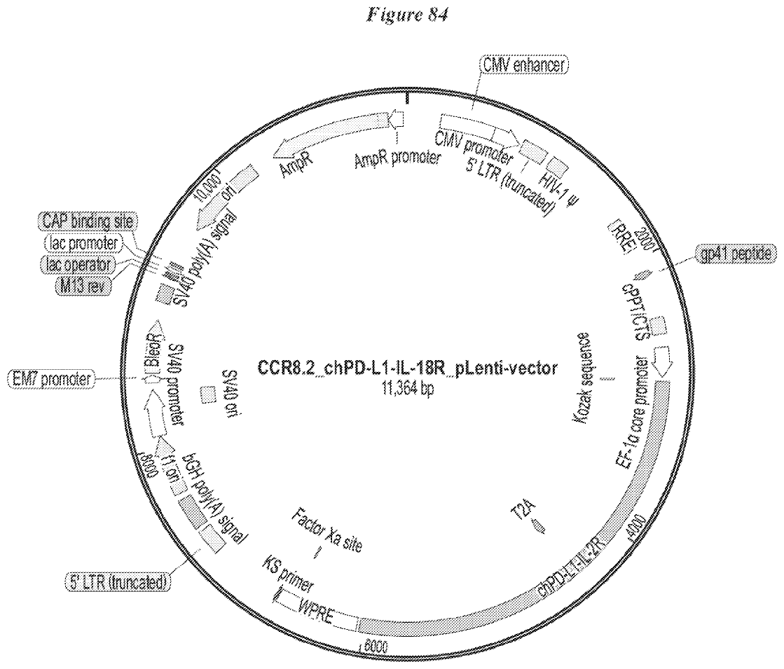

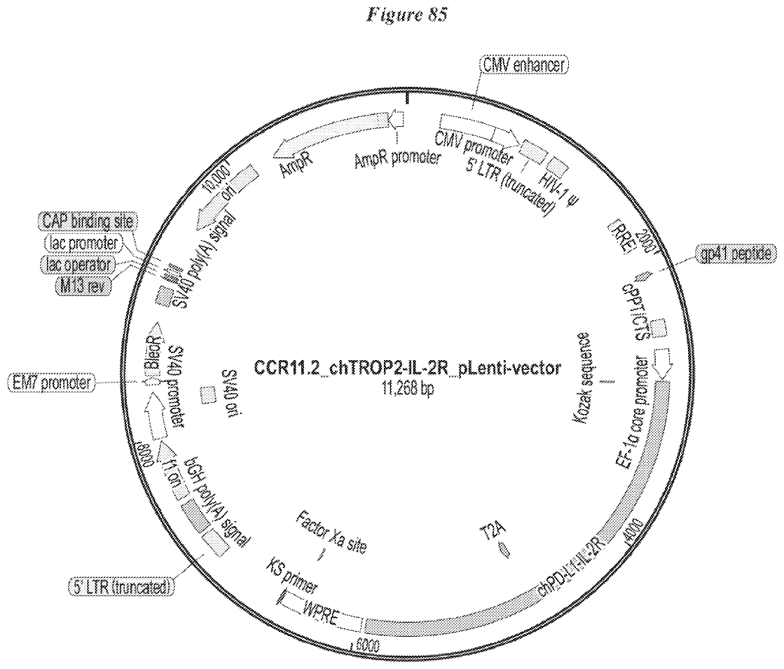

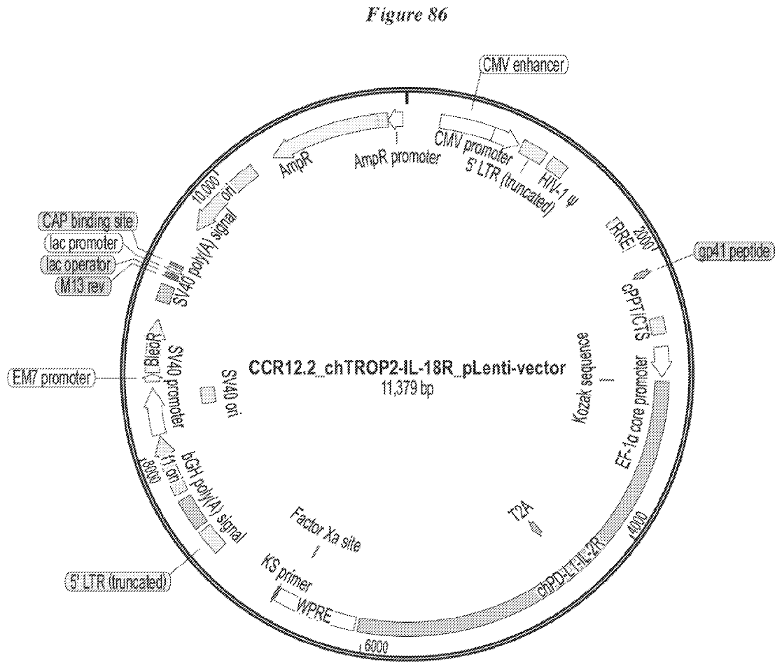

Vector maps for exemplary vectors encoding the biepitope CCRs designated CCR7.2, CCR8.2, CCR11.2, and CCR12.2, corresponding to SEQ ID NO:685 to SEQ ID NO:688, are presented in FIGS. 83 to 86.

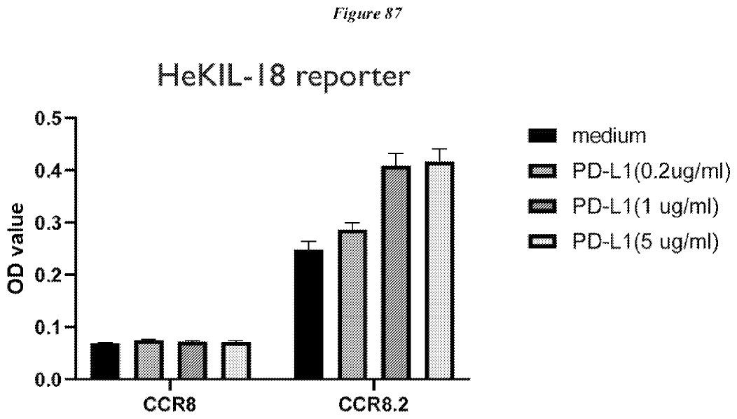

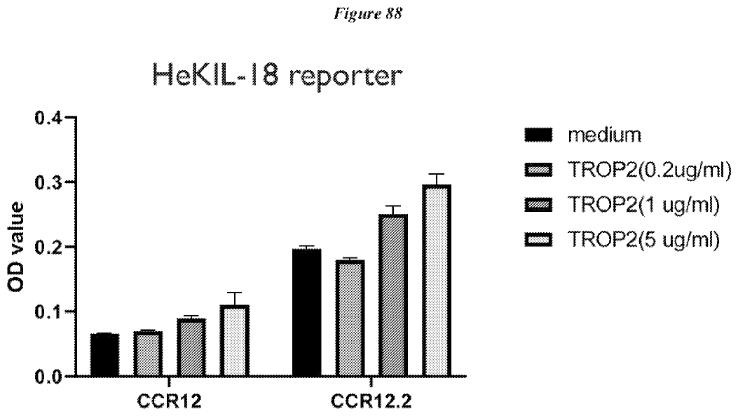

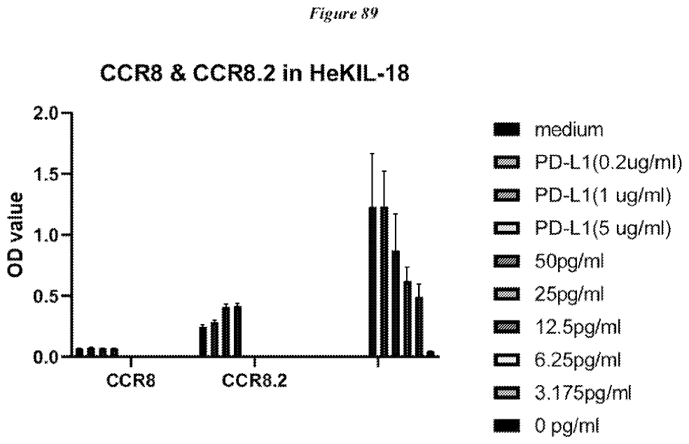

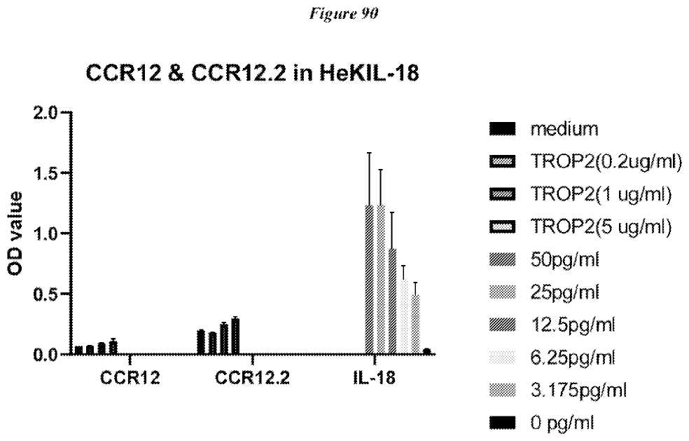

The biological function of the CCR constructs was tested with Hek-IL-18 SEAP reporter lines. CCR8 and CCR8.2 transduced HekIL-18 reporter cells (5×10⁴) were stimulated with the indicated concentration of PD-L1 (His tagged) in the presence of anti-His antibody (antibody:protein ratio=2:1; e.g., for 5 μg/mL PD-L1, add 10 μg/mL anti-His antibody). After 24 hours, supernatants were harvested, and SEAP level was determined with a spectrophotometer at 650 nm after adding Quanti-blue solution. Results are shown in FIG. 87. CCR12 and CCR12.2 transduced HekIL-18 reporter cells (5×10⁴) were stimulated with the indicated concentration of TROP-2 (His tagged) in the presence of anti-His antibody (antibody:protein ratio=2:1). After 24 hours, supernatants were harvested, and SEAP level was determined with a spectrophotometer at 650 nm after adding Quanti-blue solution. Results are shown in FIG. 88. The results for both sets of experiments are further compared to an IL-18 standard in FIG. 89 (for PD-L1) and FIG. 90 (for TROP-2), where it is shown that an OD value of 0.5 is equivalent to about 3.175 pg/mL IL-18 stimulation, demonstrating tailored activation.

Finally, expression of TROP-2 on cancer cell lines CaO-V3 and MCF-7 were assessed and quantified by flow with PE fluorescence quantitation kit, in comparison to isotype controls. TROP-2 was expressed on CaO-V3 at a level of approximately 800,000 TROP-2 molecules per cell, and on MCF-7 at a level of approximately 100,000 TROP-2 molecules per cell.

The foregoing examples are also embodiments of the present invention. Further embodiments of present invention include the sequences of SEQ ID NO:677, SEQ ID NO:678, SEQ ID NO:679, and SEQ ID NO:680, or conservative amino acid substitutions thereof, or fragments, variants, or derivatives thereof, or an amino acid sequence that is at least 99% identical to the sequence given in SEQ ID NO:677, SEQ ID NO:678, SEQ ID NO:679, or SEQ ID NO:680, at least 98% identical to the sequence given in SEQ ID NO:677, SEQ ID NO:678, SEQ ID NO:679, or SEQ ID NO:680, at least 97% identical to the sequence given in SEQ ID NO:677, SEQ ID NO:678, SEQ ID NO:679, or SEQ ID NO:680, at least 96% identical to the sequence given in SEQ ID NO:677, SEQ ID NO:678, SEQ ID NO:679, and SEQ ID NO:680, at least 95% identical to the sequence given in SEQ ID NO:677, SEQ ID NO:678, SEQ ID NO:679, and SEQ ID NO:680, at least 90% identical to the sequence given in SEQ ID NO:677, SEQ ID NO:678, SEQ ID NO:679, and SEQ ID NO:680, at least 85% identical to the sequence given in SEQ ID NO:677, SEQ ID NO:678, SEQ ID NO:679, and SEQ ID NO:680, or at least 80% identical to the sequence given in SEQ ID NO:677, SEQ ID NO:678, SEQ ID NO:679, and SEQ ID NO:680.

Further embodiments of present invention include the sequences of SEQ ID NO:681, SEQ ID NO:682, SEQ ID NO:683, and SEQ ID NO:684, or fragments, variants, or derivatives thereof, or a nucleotide sequence that is at least 99% identical to the sequence given in SEQ ID NO:681, SEQ ID NO:682, SEQ ID NO:683, and SEQ ID NO:684, at least 98% identical to the sequence given in SEQ ID NO:681, SEQ ID NO:682, SEQ ID NO:683, and SEQ ID NO:684, at least 97% identical to the sequence given in SEQ ID NO:681, SEQ ID NO:682, SEQ ID NO:683, and SEQ ID NO:684, at least 96% identical to the sequence given in SEQ ID NO:681, SEQ ID NO:682, SEQ ID NO:683, and SEQ ID NO:684, at least 95% identical to the sequence given in SEQ ID NO:681, SEQ ID NO:682, SEQ ID NO:683, and SEQ ID NO:684, at least 90% identical to the sequence given in SEQ ID NO:681, SEQ ID NO:682, SEQ ID NO:683, and SEQ ID NO:684, at least 85% identical to the sequence given in SEQ ID NO:681, SEQ ID NO:682, SEQ ID NO:683, and SEQ ID NO:684, or at least 80% identical to the sequence given in SEQ ID NO:681, SEQ ID NO:682, SEQ ID NO:683, and SEQ ID NO:684.

Further embodiments of present invention include the sequences of SEQ ID NO:685, SEQ ID NO:686, SEQ ID NO:687, and SEQ ID NO:688, or fragments, variants, or derivatives thereof, or a nucleotide sequence that is at least 99% identical to the sequence given in SEQ ID NO:685, SEQ ID NO:686, SEQ ID NO:687, and SEQ ID NO:688, at least 98% identical to the sequence given in SEQ ID NO:685, SEQ ID NO:686, SEQ ID NO:687, and SEQ ID NO:688, at least 97% identical to the sequence given in SEQ ID NO:685, SEQ ID NO:686, SEQ ID NO:687, and SEQ ID NO:688, at least 96% identical to the sequence given in SEQ ID NO:685, SEQ ID NO:686, SEQ ID NO:687, and SEQ ID NO:688, at least 95% identical to the sequence given in SEQ ID NO:685, SEQ ID NO:686, SEQ ID NO:687, and SEQ ID NO:688, at least 90% identical to the sequence given in SEQ ID NO:685, SEQ ID NO:686, SEQ ID NO:687, and SEQ ID NO:688, at least 85% identical to the sequence given in SEQ ID NO:685, SEQ ID NO:686, SEQ ID NO:687, and SEQ ID NO:688, or at least 80% identical to the sequence given in SEQ ID NO:685, SEQ ID NO:686, SEQ ID NO:687, and SEQ ID NO:688.

### Example 24: Chimeric Costimulatory Receptors with 4-1BB Intracellular Domains

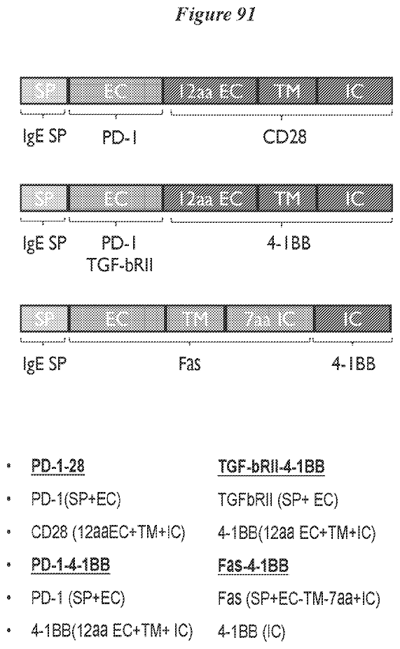

Using the methods described above, additional CCRs may be prepared. FIG. 91 shows constructs for CCRs designated CCR13, CCR14, CCR15, and CCR16. Lentivirus is prepared as described previously. Briefly, CCRs were cloned into the pLenti-IRES-EGFP plasmid. AfeI/EcoRI enzyme recognition sequences were added on the two sides of synthesized DNA sequences of the CCRs. Then, full DNA sequences were inserted into pLenti-IRES-EGFP viral vector (for CCR13-CCR16 as well as CCR17-CCR19 in Example 25). The pLenti viral vector is a self-inactivating lentiviral vector with gene expression driven by an EF-1α core promoter. To make lentivirus, pLenti vectors and helper vectors (VSV-G, Gag/Pol) were co-transfected into 293T cells. Virus supernatant was collected from day 2 or 3 culture supernatants and followed by ultracentrifugation to concentrate lentivirus for TIL transduction. Amino acid sequences for these CCRs are set forth in Table 90.

TABLE 90
Amino acid sequences of exemplary CCRs designated CCR13, CCR14, CCR15,
and CCR16.

| Identifier | Sequence (One-Letter Amino Acid Symbols) | |
|---|---|---|
| SEQ ID NO: 689 | MDWTWILFLV AAATRVHSQV TDINSKGLEL RKTVTTVETQ NLEGLHHDGQ FCHKPCPPGE | 60 |
| CCR13: ch Fas-4- | RKARDCTVNG DEPDCVPCQE GKEYTDKAHF SSKCRRCRLC DEGHGLEVEI NCTRTQNTKC | 120 |
| 1BB | RCKPNFFCNS TVCEHCDPCT KCEHGIIKEC TLTSNTKCKE EGSRSNLGWL CLLLLPIPLI | 180 |
| | VWVKRKEVQK KRGRKKLLYI FKQPFMRPVQ TTQEEDGCSC RFPEEEEGGC EL | 232 |
| SEQ ID NO: 690 | MDWTWILFLV AAATRVHSFL DSPDRPWNPP TESPALLVVT EGDNATFTCS FSNTSESFVL | 60 |
| CCR14: ch PD-1- | NWYRMSPSNQ TDKLAAFPED RSQPGQDCRF RVTQLPNGRD FHMSVVRARR NDSGTYLCGA | 120 |
| 4-1BB | ISLAPKAQIK ESLRAELRVT ERRAEVPTAH PAPAREPGHS PQIISFFLAL TSTALLFLLF | 180 |
| | FLTLRFSVVK RGRKKLLYIF KQPFMRPVQT TQEEDGCSCR FPEEEEGGCE L | 231 |
| SEQ ID NO: 691 | MDWTWILFLV AAATRVHSTI PPHVQKSVNN DMIVTDNNGA VKFPQLCKFC DVRFSTCDNQ | 60 |
| CCR15: ch | KSCMSNCSIT SICEKPQEVC VAVWRKNDEN ITLETVCHDP KLPYHDFILE DAASPKCIMK | 120 |
| TGFbRII-4-1BB | EKKKPGETFF MCSCSSDECN DNIIFSEEYN TSNPDLLLVI FQPAPAREPG HSPQIISFFL | 180 |
| | ALTSTALLFL LFFLTLRFSV VKRGRKKLLY IFKQPFMRPV QTTQEEDGCS CRFPEEEEGG | 240 |
| | CEL | 243 |
| SEQ ID NO: 692 | MDWTWILFLV AAATRVHSFL DSPDRPWNPP TESPALLVVT EGDNATFTCS FSNTSESFVL | 60 |
| CCR16: ch PD-1- | NWYRMSPSNQ TDKLAAFPED RSQPGQDCRF RVTQLPNGRD FHMSVVRARR NDSGTYLCGA | 120 |
| CD28 | ISLAPKAQIK ESLRAELRVT ERRAEVPTAH CPSPLFPGPS KPFWVLVVVG GVLACYSLLV | 180 |
| | TVAFIIFWVR SKRSRLLHSD YMNMTPRRPG PTRKHYQPYA PPRDFAAYRS | 230 |
| SEQ ID NO: 693 | QVTDINSKGL ELRKTVTTVE TQNLEGLHHD GQFCHKPCPP GERKARDCTV NGDEPDCVPC | 60 |
| FAS binding | QEGKEYTDKA HFSSKCRRCR LCDEGHGLEV EINCTRTQNT KCRCKPNFFC NSTVCEHCDP | 120 |
| domain | CTKCEHGIIK ECTLESNTKC KEEGSRSNLG WLCLLLLPIP LIVWVKRKEV QK | 172 |
| SEQ ID NO: 694 | STIPPHVQKS VNNDMIVTDN NGAVKFPQLC KFCDVRFSTC DNQKSCMSNC SITSICEKPQ | 60 |
| TGEbRII binding | EVCVAVWRKN DENISLETVC HDPKLPYHDF ILEDAASPKC IMKEKKKPGE TFFMCSCSSD | 120 |
| domain | ECNDNIIFSE EYNTSNPDLL LVIFQ | 145 |

Suitable, non-limiting embodiments of nucleotides encoding the CCRs prepared according to this example and useful as CCR constructs of the present invention are set forth in Table 91.

TABLE 91
Nucleotide sequences of exemplary CCRs designated CCR13, CCR14, CCR15,
and CCR16.

| Identifier | Sequence (One-Letter Nucleotide Symbols) | |
|---|---|---|
| SEQ ID NO: 695 | ATGGACTGGA CCTGGATTCT GTTCCTGGTG GCCGCCGCTA CAAGAGTGCA CAGCCAGGTG | 60 |
| CCR13: ch Fas-4- | ACCGACATCA ACTCCAAGGG CCTGGAACTG AGAAAGACCG TGACAACCGT TGAGACACAA | 120 |
| 1BB | AATCTGGAAG GCCTGCACCA CGACGGCCAG TTTTGCCACA AGCCTTGTCC TCCAGGCGAG | 180 |
| | CGGAAGGCCA GAGATTGCAC CGTGAACGGC GATGAGCCTG ACTGCGTGCC ATGTCAGGAG | 240 |
| | GGCAAGGAAT ACACCGATAA GGCCCACTTC AGCAGCAAGT GCAGGCGGTG CCGGCTGTGC | 300 |
| | GACGAGGGCC ACGGACTGGA AGTGGAAATC AACTGCACCA GAACACAGAA CACAAAGTGC | 360 |
| | AGATGCAAGC CCAACTTCTT CTGCAACAGC ACCGTGTGCG AGCATTGTGA CCCCTGCACA | 420 |
| | AAATGTGAAC ACGGCATCAT CAAGGAGTGC ACCCTGACCA GCAACACCAA GTGTAAAGAG | 480 |
| | GAAGGAAGCA GAAGCAATCT GGGCTGGCTG TGTCTGCTCC TGCTGCCTAT TCCTCTGATC | 540 |
| | GTGTGGGTCA AGAGAAAAGA GGTGCAGAAG AAGCGGGGCA GAAAGAAACT GCTGTACATC | 600 |
| | TTCAAGCAGC CTTTTATGCG GCCTGTGCAG ACCACTCAGG AGGAGGACGG CTGCAGCTGC | 660 |
| | AGATTCCCCG AGGAAGAGGA AGGCGGATGT GAGCTG | 696 |
| SEQ ID NO: 696 | ATGGACTGGA CCTGGATTCT GTTCCTGGTC GCCGCCGCCA CAAGAGTGCA TTCTTTTCTG | 60 |
| CCR14: ch PD-1- | GACAGCCCTG ATAGACCTTG GAACCCTCCA ACCTTTTCCC CCGCCCTGCT GGTGGTGACC | 120 |
| 4-1BB | GAGGGCGACA ACGCCACCTT CACCTGTAGC TTTTCCAATA CCAGCGAGAG CTTCGTGCTG | 180 |
| | AACTGGTACA GAATGTCTCC TAGCAACCAG ACCGACAAGC TGGCCGCTTT TCCTGAGGAC | 240 |
| | AGATCCCAGC CCGGCCAGGA CTGCAGATTC AGAGTGACCC AGCTGCCTAA CGGCAGAGAT | 300 |
| | TTCCACATGA GCGTGGTGAG AGCCAGAAGA AACGACAGCG GCACCTACCT GTGCGGAGCC | 360 |
| | ATCAGCCTGG CTCCTAAGGC CCAGATTAAG GAATCTCTGA GAGCCGAGCT GAGGGTGACA | 420 |
| | GAGAGAAGAG CTGAAGTGCC TACAGCCCAC CCCGCCCCAG CTCGCGAGCC TGGACACAGC | 480 |
| | CCTCAGATCA TCTCTTTCTT CCTGGCCCTG ACCAGCACCG CCCTGCTCTT CCTGCTCTTC | 540 |
| | TTCCTGACAC TGCGGTTCAG CGTTGTGAAA CGGGGCCGAA AAAAGCTGCT GTACATCTTC | 600 |
| | AAGCAGCCTT TCATGCGGCC CGTGCAAACA ACACAGGAGG AAGATGGCTG CAGCTGCCGG | 660 |
| | TTCCCCGAGG AAGAGGAAGG CGGCTGTGAA CTG | 693 |
| SEQ ID NO: 697 | ATGGACTGGA CCTGGATTCT GTTTCTGGTC GCCGCCGCTA CAAGAGTTCA CAGCACCATC | 60 |
| CCR15: ch | CCTCCTCATG TGCAAAAATC CGTGAACAAC GACATGATCG TGACCGACAA TAACGGCGCC | 120 |
| TGFbRII-4-1BB | GTCAAATTCC CCCAGCTGTG CAAGTTCTGC GACGTGCGGT TTTCTACATG TGATAACCAG | 180 |
| | AAGTCCTGCA TGAGCAACTG CAGCATCACA AGCATCTGCG AGAAACCTCA GGAGGTGTGC | 240 |
| | GTGGCCGTGT GGCGGAAGAA CGACGAGAAC ATCACCCTGG AAACCGTGTG TCACGACCCC | 300 |
| | AAGCTGCCTT ACCACGACTT CATCCTGGAA GATGCCGCCT CTCCAAAGTG CATCATGAAG | 360 |
| | GAAAAGAAAA AGCCCGGCGA GACCTTCTTC ATGTGCTCTT GTTCTAGCGA TGAGTGCAAT | 420 |
| | GATAACATCA TTTTCAGCGA GGAATACAAC ACCAGCAATC CCGACCTGCT GCTCGTGATC | 480 |
| | TTTCAGCCCG CCCCTGCTAG AGAGCCTGGA CACTCCCCTC AGATCATCAG CTTCTTCCTG | 540 |
| | GCCCTGACAA GCACAGCCCT GCTGTTTCTG CTGTTCTTCC TGACCCTGAG ATTCAGCGTG | 600 |
| | GTGAAGCGGG GAAGAAAGAA GCTGCTGTAC ATCTTCAAGC AGCCTTTCAT GCGCCCTGTG | 660 |
| | CAGACCACCC AGGAGGAGGA CGGCTGCAGC TGCAGATTCC CAGAGGAAGA GGAAGGCGGC | 720 |
| | TGTGAACTG | 729 |
| SEQ ID NO: 698 | ATGGACTGGA CCTGGATTCT GTTTCTGGTG GCCGCCGCTA CAAGAGTGCA CAGCTTCCTG | 60 |
| CCR16: ch PD-1- | GATTCTCCAG ACCGGCCTTG GAACCCCCCC ACCTTCTCCC CAGCCCTGCT GGTGGTGACA | 120 |
| CD28 | GAGGGCGACA ACGCCACCTT CACATGCAGC TTTTCTAATA CCAGCGAGAG CTTCGTGCTG | 180 |
| | AATTGGTACA GAATGTCCCC TAGCAACCAG ACCGACAAGC TGGCCGCTTT TCCTGAGGAC | 240 |
| | AGATCTCAGC CTGGACAGGA TTGCAGATTC AGAGTGACCC AGCTGCCTAA CGGCAGAGAC | 300 |
| | TTCCACATGA GCGTGGTCAG AGCCAGACGG AACGACAGCG GAACATATCT GTGCGGCGCC | 360 |
| | ATCAGCCTGG CCCCZAAGGC CCAAATCAAG GAAAGCCTGA GAGCTGAACT GAGGGTTACC | 420 |
| | GAGCGGCGGG CCGAAGTGCC CACAGCCCAC TGCCCTAGCC CTCTGTTCCC CGGCCCCAGC | 480 |
| | AAACCTTTCT GGGTCCTGGT GGTGGTGGGC GGCGTGCTGG CTTGTTACAG CCTCCTGGTG | 540 |
| | ACCGTGGCCT TCATCATCTT CTGGGTGCGG AGCAAGCGGA GTAGACTGCT GCATTCTGAT | 600 |
| | TACATGAACA TGACCCCTAG ACGGCCTGGC CCTACCAGAA AGCACTACCA GCCCTACGCC | 660 |
| | CCTCCAAGAG ATTTCGCCGC CTACCGCTCC | 690 |

Vectors encoding the CCRs designated CCR13, CCR14, CCR15, and CCR16 were prepared as described above. The full nucleotide sequences of these vectors are presented in Table 92.

TABLE 92
Nucleotide sequences of exemplary vectors encoding CCRs designated CCR13,
CCR14, CCR15, and CCR16.

| Identifier | Sequence (One-Letter Nucleotide Symbols) | |
|---|---|---|
| SEQ ID NO: 699 | GTCGACGGAT CGGGAGATCT CCCGATCCCC TATGGTGCAC TCTCAGTACA ATCTGCTCTG | 60 |
| CCR13: ch Fas-4- | ATGCCGCATA GTTAAGCCAG TATCTGCTCC CTGCTTGTGT GTTGGAGGTC GCTGAGTAGT | 120 |
| 1BB | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGASTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCAEGGACT GGACCTGGAT TCTGTTCCTG GTGGCCGCCG CTACAAGAGT | 2940 |
| | GCACAGCCAG GTGACCGACA TCAACTCCAA GGGCCTGGAA CTGAGAAAGA CCGTGACAAC | 3000 |
| | CGTTGAGACA CAAAATCTGG AAGGCCTGCA CCACGACGGC CAGTTTTGCC ACAAGCCTTG | 3060 |
| | TCCTCCAGGC GAGCGGAAGG CCAGAGATTG CACCGTGAAC GGCGATGAGC CTGACTGCGT | 3120 |
| | GCCATGTCAG GAGGGCAAGG AATACACCGA TAAGGCCCAC TTCAGCAGCA AGTGCAGGCG | 3180 |
| | GTGCCGGCTG TGCGACGAGG GCCACGGACT GGAAGTGGAA ATCAACTGCA CCAGAACACA | 3240 |
| | GAACACAAAG TGCAGATGCA AGCCCAACTT CTTCTGCAAC AGCACCGTGT GCGAGCATTG | 3300 |
| | TGACCCCTGC ACAAAATGTG AACACGGCAT CATCAAGGAG TGCACCCTGA CCAGCAACAC | 3360 |
| | CAAGTGTAAA GAGGAAGGAA GCAGAAGCAA TCTGGGCTGG CTGTGTCTGC TCCTGCTGCC | 3420 |
| | TATTCCTCTG ATCGTGTGGG TCAAGAGAAA AGAGGTGCAG AAGAAGCGGG GCAGAAAGAA | 3480 |
| | ACTGCTGTAC ATCTTCAAGC AGCCTTTTAT GCGGCCTGTG CAGACCACTC AGGAGGAGGA | 3540 |
| | CGGCTGCAGC TGCAGATTCC CCGAGGAAGA GGAAGGCGGA TGTGAGCTGT GAGAATTCCG | 3600 |
| | CCCCCCCCCC CCCCCCCCCT CTCCCTCCCC CCCCCCTAAC GTTACTGGCC GAAGCCGCTT | 3660 |
| | GGAATAAGGC CGGTGTGCGT TTGTCTATAT GTTATTTTCC ACCATATTGC CGTCTTTTGG | 3720 |
| | CAATGTGAGG GCCCGGAAAC CTGGCCCTGT CTTCTTGACG AGCATTCCTA GGGGTCTTTC | 3780 |
| | CCCTCTCGCC AAAGGAATGC AAGGTCTGTT GAATGTCGTG AAGGAAGCAG TTCCTCTGGA | 3840 |
| | AGCTTCTTGA AGACAAACAA CGTCTGTAGC GACCCTTTGC AGGCAGCGGA ACCCCCCACC | 3900 |
| | TGGCGACAGG TGCCTCTGCG GCCAAAAGCC ACGTGTATAA GATACACCTG CAAAGGCGGC | 3960 |
| | ACAACCCCAG TGCCACGTTG TGAGTTGGAT AGTIGTGGAA AGAGTCAAAT GGCTCTCCTC | 4020 |
| | AAGCGTATTC AACAAGGGGC TGAAGGATGC CCAGAAGGTA CCCCATTGTA TGGGATCTGA | 4080 |
| | TCTGGGGCCT CGGTGCACAT GCTTTACATG TGTTTAGTCG AGGTTAAAAA AACGTCTAGG | 4140 |
| | CCCCCCGAAC CACGGGGACG TGGTTTTCCT TTGAAAAACA CGATGATAAG CTTGATCACG | 4200 |
| | CGTGCCACCA TGAGCAAGGG CGAGGAGCTG TTCACCGGGG TGGTGCCCAT CCTGGTCGAG | 4260 |
| | CTGGACGGCG ACGTAAACGG CCACAAGTTC AGCGTGTCCG GCGAGGGCGA GGGCGATGCC | 4320 |
| | ACCTACGGCA AGCTGACCCT GAAGTTCATC TGCACCACCG GCAAGCTGCC CGTGCCCTGG | 4380 |
| | CCCACCCTCG TGACCACCCT GACCTACGGC GTGCAGTGCT TCAGCCGCTA CCCCGACCAC | 4440 |
| | ATGAAGCAGC ACGACTTCTT CAAGTCCGCC ATGCCCGAAG GCTACGTCCA GGAGCGCACC | 4500 |
| | ATCTTCTTCA AGGACGACGG CAACTACAAG ACCCGCGCCG AGGTGAAGTT CGAGGGCGAC | 4560 |
| | ACCCTGGTGA ACCGCATCGA GCTGAAGGGC ATCGACTTCA AGGAGGACGG CAACATCCTG | 4620 |
| | GGGCACAAGC TGGAGTACAA CTACAACAGC CACAACGTCT ATATCATGGC CGACAAGCAG | 4680 |
| | AAGAACGGCA TCAAGGTGAA CTTCAAGATC CGCCACAACA TCGAGGACGG CAGCGTGCAG | 4740 |
| | CTCGCCGACC ACTACCAGCA GAACACCCCC ATCGGCGACG GCCCCGTGCT GCTGCCCGAC | 4800 |
| | AACCACTACC TGAGCACCCA GTCCGCCCTG AGCAAAGACC CCAACGAGAA GCGCGATCAC | 4860 |
| | ATGGTCCTGC TGGAGTTCGT GACCGCCGCC GGGATCACTC ACGGCATGGA CGAGCTGTAC | 4920 |
| | AAGTGAGAAT TCGATATCAA GCTTATCGGT AATCAACCTC TGGATTACAA AATTTGTGAA | 4980 |
| | AGATTGACTG GTATTCTTAA CTATGTTGCT CCTTTTACGC TATGTGGATA CGCTGCTTTA | 5040 |
| | ATGCCTTTGT ATCATGCTAT TGCTTCCCGT ATGGCTTTCA TTTTCTCCTC CTTGTATAAA | 5100 |
| | TCCTGGTTGC TGTCTCTTTA TGAGGAGTTG TGGCCCGTTG TCAGGCAACG TGGCGTGGTG | 5160 |
| | TGCACTGTGT TTGCTGACGC AACCCCCACT GGTTGGGGCA TTGCCACCAC CTGTCAGCTC | 5220 |
| | CTTTCCGGGA CTTTCGCTTT CCCCCTCCCT ATTGCCACGG CGGAACTCAT CGCCGCCTGC | 5280 |
| | CTTGCCCGCT GCTGGACAGG GGCTCGGCTG TTGGGCACTG ACAATTCCGT GGTGTTGTCG | 5340 |
| | GGGAAATCAT CGTCCTTTCC TTGGCTGCTC GCCTGTGTTG CCACCTGGAT TCTGCGCGGG | 5400 |
| | ACGTCCTTCT GCTACGTCCC TTCGGCCCTC AATCCAGCGG ACCTTCCTTC CCGCGGCCTG | 5460 |
| | CTGCCGGCTC TGCGGCCTCT TCCGCGTCTT CGCCTTCGCC CTCAGACGAG TCGGATCTCC | 5520 |
| | CTTTGGGCCG CCTCCCCGCA TCGATACCGT CGACCTCGAG ACCTAGAAAA ACATGGAGCA | 5580 |
| | ATCACAAGTA GCAATACAGC AGCTACCAAT GCTGATTGTG CCTGGCTAGA AGCACAAGAG | 5640 |
| | GAGGAGGAGG TGGGTTTTCC AGTCACACCT CAGGTACCTT TAAGACCAAT GACTTACAAG | 5700 |
| | GCAGCTGTAG ATCTTAGCCA CTTTTTAAAA GAAAAGGGGG GACTGGAAGG GCTAATTCAC | 5760 |
| | TCCCAACGAA GACAAGATAT CCTTGATCTG TGGATCTACC ACACACAAGG CTACTTCCCT | 5820 |
| | GATTGGCAGA ACTACACACC AGGGCCAGGG ATCAGATATC CACTGACCTT TGGATGGTGC | 5880 |
| | TACAAGCTAG TACCAGTTGA GCAAGAGAAG GTAGAAGAAG CCAATGAAGG AGAGAACACC | 5940 |
| | CGCTTGTTAC ACCCTGTGAG CCTGCATGGG ATGGATGACC CGGAGAGAGA AGTATTAGAG | 6000 |
| | TGGAGGTTTG ACAGCCGCCT AGCATTTCAT CACATGGCCC GAGAGCTGCA TCCGGACTGT | 6060 |
| | ACTGGGTCTC TCTGGTTAGA CCAGATCTGA GCCTGGGAGC TCTCTGGCTA ACTAGGGAAC | 6120 |
| | CCACTGCTTA AGCCTCAATA AAGCTTGCCT TGAGTGCTTC AAGTAGTGTG TGCCCGTCTG | 6180 |
| | TTGTGTGACT CTGGTAACTA GAGATCCCTC AGACCCTTTT AGTCAGTGTG GAAAATCTCT | 6240 |
| | AGCAGGGCCC GTTTAAACCC GCTGATCAGC CTCGACTGTG CCTTCTAGTT GCCAGCCATC | 6300 |
| | TGTTGTTTGC CCCTCCCCCG TGCCTTCCTT GACCCTGGAA GGTGCCACTC CCACTGTCCT | 6360 |
| | TTCCTAATAA AATGAGGAAA TTGCATCGCA TTGTCTGAGT AGGTGTCATT CTATTCTGGG | 6420 |
| | GGGTGGGGTG GGGCAGGACA GCAAGGGGGA GGATTGGGAA GACAATAGCA GGCATGCTGG | 6480 |
| | GGATGCGGTG GGCTCTATGG CTTCTGAGGC GGAAAGAACC AGCTGGGGCT CTAGGGGGTA | 6540 |
| | TCCCCACGCG CCCTGTAGCG GCGCATTAAG CGCGGCGGGT GTGGTGGTTA CGCGCAGCGT | 6600 |
| | GACCGCTACA CTTGCCAGCG CCCTAGCGCC CGCTCCTTTC GCTTTCTTCC CTTCCTTTCT | 6660 |
| | CGCCACGTTC GCCGGCTTTC CCCGTCAAGC TCTAAATCGG GGGCTCCCTT TAGGGTTCCG | 6720 |
| | ATTTAGTGCT TTACGGCACC TCGACCCCAA AAAACTTGAT TAGGGTGATG GTTCACGTAG | 6780 |
| | TGGGCCATCG CCCTGATAGA CGGTTTTTCG CCCTTTGACG TTGGAGTCCA CGTTCTTTAA | 6840 |
| | TAGTGGACTC TTGTTCCAAA CTGGAACAAC ACTCAACCCT ATCTCGGTCT ATTCTTTTGA | 6900 |
| | TTTATAAGGG ATTTTGCCGA TTTCGGCCTA TTGGTTAAAA AATGAGCTGA TTTAACAAAA | 6960 |
| | ATTTAACGCG AATTAATTCT GTGGAATGTG TGTCAGTTAG GGTGTGGAAA GTCCCCAGGC | 7020 |
| | TCCCCAGCAG GCAGAAGTAT GCAAAGCATG CATCTCAATT AGTCAGCAAC CAGGTGTGGA | 7080 |
| | AAGTCCCCAG GCTCCCCAGC AGGCAGAAGT ATGCAAAGCA TGCATCTCAA TTAGTCAGCA | 7140 |
| | ACCATAGTCC CGCCCCTAAC TCCGCCCATC CCGCCCCTAA CTCCGCCCAG TTCCGCCCAT | 7200 |
| | TCTCCGCCCC ATGGCTGACT AATTTTTTTT ATTTATGCAG AGGCCGAGGC CGCCTCTGCC | 7260 |
| | TCTGAGCTAT TCCAGAAGTA GTGAGGAGGC TTTTTTGGAG GCCTAGGCTT TTGCAAAAAG | 7320 |
| | CTCCCGGGAG CTTGTATATC CATTTTCGGA TCTGATCAGC ACGTGTTGAC AATTAATCAT | 7380 |
| | CGGCATAGTA TATCGGCATA GTATAATACG ACAAGGTGAG GAACTAAACC ATGGCCAAGT | 7440 |
| | TGACCAGTGC CGTTCCGGTG CTCACCGCGC GCGACGTCGC CGGAGCGGTC GAGTTCTGGA | 7500 |
| | CCGACCGGCT CGGGTTCTCC CGGGACTTCG TGGAGGACGA CTTCGCCGGT GTGGTCCGGG | 7560 |
| | ACGACGTGAC CCTGETCATC AGCGCGGTCC AGGACCAGGT GGTGCCGGAC AACACCCTGG | 7620 |
| | CCTGGGTGTG GGTGCGCGGC CTGGACGAGC TGTACGCCGA GTGGTCGGAG GTCGTGTCCA | 7680 |
| | CGAACTTCCG GGACGCCTCC GGGCCGGCCA TGACCGAGAT CGGCGAGCAG CCGTGGGGGC | 7740 |
| | GGGAGTTCGC CCTGCGCGAC CCGGCCGGCA ACTGCGTGCA CTTCGTGGCC GAGGAGCAGG | 7800 |
| | ACTGACACGT GCTACGAGAT TTCGATTCCA CCGCCGCCTT CTATGAAAGG TTGGGCTTCG | 7860 |
| | GAATCGTTTT CCGGGACGCC GGCTGGATGA TCCTCCAGCG CGGGGATCTC ATGCTGGAGT | 7920 |
| | TCTTCGCCCA CCCCAACTTG TTTATTGCAG CTTATAATGG TTACAAATAA AGCAATAGCA | 7980 |
| | TCACAAATTT CACAAATAAA GCATTTTTTT CACTGCATTC TAGTTGTGGT TTGTCCAAAC | 8040 |
| | TCATCAATGT ATCTTATCAT GTCTGTATAC CGTCGACCTC TAGCTAGAGC TTGGCGTAAT | 8100 |
| | CATGGTCATA GCTGETTCCT GTGTGAAATT GTTATCCGCT CACAATTCCA CACAACATAC | 8160 |
| | GAGCCGGAAG CATAAAGTGT AAAGCCTGGG GTGCCTAATG AGTGAGCTAA CTCACATTAA | 8220 |
| | TTGCGTTGCG CTCACTGCCC GCTTTCCAGT CGGGAAACCT GTCGTGCCAG CTGCATTAAT | 8280 |
| | GAATCGGCCA ACGCGCGGGG AGAGGCGGTT TGCGTATTGG GCGCTCTTCC GCTTCCTCGC | 8340 |
| | TCACTGACTC GCTGCGCTCG GTCGTTCGGC TGCGGCGAGC GGTATCAGCT CACTCAAAGG | 8400 |
| | CGGTAATACG GTTATCCACA GAATCAGGGG ATAACGCAGG AAAGAACATG TGAGCAAAAG | 8460 |
| | GCCAGCAAAA GGCCAGGAAC CGTAAAAAGG CCGCGTTGCT GGCGTTTTTC CATAGGCTCC | 8520 |
| | GCCCCCCTGA CGAGCATCAC AAAAATCGAC GCTCAAGTCA GAGGTGGCGA AACCCGACAG | 8580 |
| | GACTATAAAG ATACCAGGCG TTTCCCCCTG GAAGCTCCCT CGTGCGCTCT CCTGTTCCGA | 8640 |
| | CCCTGCCGCT TACCGGATAC CTGTCCGCCT TTCTCCCTTC GGGAAGCGTG GCGCTTTCTC | 8700 |
| | ATAGCTCACG CTGTAGGTAT CTCAGTTCGG TGTAGGTCGT TCGCTCCAAG CTGGGCTGTG | 8760 |
| | TGCACGAACC CCCCGTTCAG CCCGACCGCT GCGCCTTATC CGGTAACTAT CGTCTTGAGT | 8820 |
| | CCAACCCGGT AAGACACGAC TTATCGCCAC TGGCAGCAGC CACTGGTAAC AGGATTAGCA | 8880 |
| | GAGCGAGGTA TGTAGGCGGT GCTACAGAGT TCTTGAAGTG GTGGCCTAAC TACGGCTACA | 8940 |
| | CTAGAAGAAC AGTATTTGGT ATCTGCGCTC TGCTGAAGCC AGTTACCTTC GGAAAAAGAG | 9000 |
| | TTGGTAGCTC TTGATCCGGC AAACAAACCA CCGCTGGTAG CGGTGGTTTT TTTGTTTGCA | 9060 |
| | AGCAGCAGAT TACGCGCAGA AAAAAAGGAT CTCAAGAAGA TCCTTTGATC TTTTCTACGG | 9120 |
| | GGTCTGACGC TCAGTGGAAC GAAAACTCAC GTTAAGGGAT TTTGGTCATG AGATTATCAA | 9180 |
| | AAAGGATCTT CACCTAGATC CTTTTAAATT AAAAATGAAG TTTTAAATCA ATCTAAAGTA | 9240 |
| | TATATGAGTA AACTTGGTCT GACAGTTACC AATGCTTAAT CAGTGAGGCA CCTATCTCAG | 9300 |
| | CGATCTGTCT ATTTCGTTCA TCCATAGTTG CCTGACTCCC CGTCGTGTAG ATAACTACGA | 9360 |
| | TACGGGAGGG CTTACCATCT GGCCCCAGTG CTGCAATGAT ACCGCGAGAC CCACGCTCAC | 9420 |
| | CGGCTCCAGA TTTATCAGCA ATAAACCAGC CAGCCGGAAG GGCCGAGCGC AGAAGTGGTC | 9480 |
| | CTGCAACTTT ATCCGCCTCC ATCCAGTCTA TTAATTGTTG CCGGGAAGCT AGAGTAAGTA | 9540 |
| | GTTCGCCAGT TAATAGTTTG CGCAACGTTG TTGCCATTGC TACAGGCATC GTGGTGTCAC | 9600 |
| | GCTCGTCGTT TGGTATGGCT TCATTCAGCT CCGGTTCCCA ACGATCAAGG CGAGTTACAT | 9660 |
| | GATCCCCCAT GTTGTGCAAA AAAGCGGTTA GCTCCTTCGG TCCTCCGATC GTTGTCAGAA | 9720 |
| | GTAAGTTGGC CGCAGTGTTA TCACTCATGG TTATGGCAGC ACTGCATAAT TCTCTTACTG | 9780 |
| | TCATGCCATC CGTAAGATGC TTTTCTGTGA CTGGTGAGTA CTCAACCAAG TCATTCTGAG | 9840 |
| | AATAGTGTAT GCGGCGACCG AGTTGCTCTT GCCCGGCGTC AATACGGGAT AATACCGCGC | 9900 |
| | CACATAGCAG AACTTTAAAA GTGCTCATCA TTGGAAAACG TTCTTCGGGG CGAAAACTCT | 9960 |
| | CAAGGATCTT ACCGCTGTTG AGATCCAGTT CGATGTAACC CACTCGTGCA CCCAACTGAT | 10020 |
| | CTTCAGCATC TTTTACTTTC ACCAGCGTTT CTGGGTGAGC AAAAACAGGA AGGCAAAATG | 10080 |
| | CCGCAAAAAA GGGAATAAGG GCGACACGGA AATGTTGAAT ACTCATACTC TTCCTTTTTC | 10140 |
| | AATATTATTG AAGCATTTAT CAGGGTTATT GTCTCATGAG CGGATACATA TTTGAATGTA | 10200 |
| | TTTAGAAAAA TAAACAAATA GGGGTTCCGC GCACATTTCC CCGAAAAGTG CCACCTGAC | 10259 |
| SEQ ID NO: 700 | GTCGACGGAT CGGGAGATCT CCCGATCCCC TATGGTGCAC TCTCAGTACA ATCTGCTCTG | 60 |
| CCR14: ch PD-1- | ATGCCGCATA GTTAAGCCAG TATCTGCTCC CTGCTTGTGT GTTGGAGGTC GCTGAGTAGT | 120 |
| 4-1BB | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGASTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCAEGGACT GGACCTGGAT TCTGTTCCTG GTCGCCGCCG CCACAAGAGT | 2940 |
| | GCATTCTTTT CTGGACAGCC CTGATAGACC TTGGAACCCT CCAACCTTTT CCCCCGCCCT | 3000 |
| | GCTGGTGGTG ACCGAGGGCG ACAACGCCAC CTTCACCTGT AGCTTTTCCA ATACCAGCGA | 3060 |
| | GAGCTTCGTG CTGAACTGGT ACAGAATGTC TCCTAGCAAC CAGACCGACA AGCTGGCCGC | 3120 |
| | TTTTCCTGAG GACAGATCCC AGCCCGGCCA GGACTGCAGA TTCAGAGTGA CCCAGCTGCC | 3180 |
| | TAACGGCAGA GATTTCCACA TGAGCGTGGT GAGAGCCAGA AGAAACGACA GCGGCACCTA | 3240 |
| | CCTGTGCGGA GCCASCAGCC TGGCTCCTAA GGCCCAGATT AAGGAATCTC TGAGAGCCGA | 3300 |
| | GCTGAGGGTG ACAGAGAGAA GAGCTGAAGT GCCTACAGCC CACCCCGCCC CAGCTCGCGA | 3360 |
| | GCCTGGACAC AGCCCTCAGA TCATCTCTTT CTTCCTGGCC CTGACCAGCA CCGCCCTGCT | 3420 |
| | CTTCCTGCTC TTCTTCCTGA CACTGCGGTT CAGCGTTGTG AAACGGGGCC GAAAAAAGCT | 3480 |
| | GCTGTACATC TTCAAGCAGC CTTTCATGCG GCCCGTGCAA ACAACACAGG AGGAAGATGG | 3540 |
| | CTGCAGCTGC CGGTTCCCCG AGGAAGAGGA AGGCGGCTGT GAACTGTGAG AATTCCGCCC | 3600 |
| | CCCCCCCCCC CCCCCCTCTC CCTCCCCCCC CCCTAACGTT ACTGGCCGAA GCCGCTTGGA | 3660 |
| | ATAAGGCCGG TGTGCGTTTG TCTATATGTT ATTTTCCACC ATATTGCCGT CTTTTGGCAA | 3720 |
| | TGTGAGGGCC CGGAAACCTG GCCCTGTCTT CTTGACGAGC ATTCCTAGGG GTCTTTCCCC | 3780 |
| | TCTCGCCAAA GGAATGCAAG GTCTGTTGAA TGTCGTGAAG GAAGCAGTTC CTCTGGAAGC | 3840 |
| | TTCTTGAAGA CAAACAACGT CTGTAGCGAC CCTTTGCAGG CAGCGGAACC CCCCACCTGG | 3900 |
| | CGACAGGTGC CTCTGCGGCC AAAAGCCACG TGTATAAGAT ACACCTGCAA AGGCGGCACA | 3960 |
| | ACCCCAGTGC CACGETGTGA GTTGGATAGT TGTGGAAAGA GTCAAATGGC TCTCCTCAAG | 4020 |
| | CGTATTCAAC AAGGGGCTGA AGGATGCCCA GAAGGTACCC CATTGTATGG GATCTGATCT | 4080 |
| | GGGGCCTCGG TGCACATGCT TTACATGTGT TTAGTCGAGG TTAAAAAAAC GTCTAGGCCC | 4140 |
| | CCCGAACCAC GGGGACGTGG TTTTCCTTTG AAAAACACGA TGATAAGCTT GATCACGCGT | 4200 |
| | GCCACCATGA GCAAGGGCGA GGAGCTGTTC ACCGGGGTGG TGCCCATCCT GGTCGAGCTG | 4260 |
| | GACGGCGACG TAAACGGCCA CAAGTTCAGC GTGTCCGGCG AGGGCGAGGG CGATGCCACC | 4320 |
| | TACGGCAAGC TGACCCTGAA GTTCATCTGC ACCACCGGCA AGCTGCCCGT GCCCTGGCCC | 4380 |
| | ACCCTCGTGA CCACCCTGAC CTACGGCGTG CAGTGCTTCA GCCGCTACCC CGACCACATG | 4440 |
| | AAGCAGCACG ACTTCTTCAA GTCCGCCATG CCCGAAGGCT ACGTCCAGGA GCGCACCATC | 4500 |
| | TTCTTCAAGG ACGACGGCAA CTACAAGACC CGCGCCGAGG TGAAGTTCGA GGGCGACACC | 4560 |
| | CTGGTGAACC GCATCGAGCT GAAGGGCATC GACTTCAAGG AGGACGGCAA CATCCTGGGG | 4620 |
| | CACAAGCTGG AGTACAACTA CAACAGCCAC AACGTCTATA TCATGGCCGA CAAGCAGAAG | 4680 |
| | AACGGCATCA AGGTGAACTT CAAGATCCGC CACAACATCG AGGACGGCAG CGTGCAGCTC | 4740 |
| | GCCGACCACT ACCAGCAGAA CACCCCCATC GGCGACGGCC CCGTGCTGCT GCCCGACAAC | 4800 |
| | CACTACCTGA GCACCCAGTC CGCCCTGAGC AAAGACCCCA ACGAGAAGCG CGATCACATG | 4860 |
| | GTCCTGCTGG AGTTCGTGAC CGCCGCCGGG ATCACTCACG GCATGGACGA GCTGTACAAG | 4920 |
| | TGAGAATTCG ATATCAAGCT TATCGGTAAT CAACCTCTGG ATTACAAAAT TTGTGAAAGA | 4980 |
| | TTGACTGGTA TTCTTAACTA TGTTGCTCCT TTTACGCTAT GTGGATACGC TGCTTTAATG | 5040 |
| | CCTTTGTATC ATGCTATTGC TTCCCGTATG GCTTTCATTT TCTCCTCCTT GTATAAATCC | 5100 |
| | TGGTTGCTGT CTCTTTATGA GGAGTTGTGG CCCGTTGTCA GGCAACGTGG CGTGGTGTGC | 5160 |
| | ACTGTGTTTG CTGACGCAAC CCCCACTGGT TGGGGCATTG CCACCACCTG TCAGCTCCTT | 5220 |
| | TCCGGGACTT TCGCTTTCCC CCTCCCTATT GCCACGGCGG AACTCATCGC CGCCTGCCTT | 5280 |
| | GCCCGCTGCT GGACAGGGGC TCGGCTGTTG GGCACTGACA ATTCCGTGGT GTTGTCGGGG | 5340 |
| | AAATCATCGT CCTTTCCTTG GCTGCTCGCC TGTGTTGCCA CCTGGATTCT GCGCGGGACG | 5400 |
| | TCCTTCTGCT ACGTCCCTTC GGCCCTCAAT CCAGCGGACC TTCCTTCCCG CGGCCTGCTG | 5460 |
| | CCGGCTCTGC GGCCTCTTCC GCGTCTTCGC CTTCGCCCTC AGACGAGTCG GATCTCCCTT | 5520 |
| | TGGGCCGCCT CCCCGCATCG ATACCGTCGA CCTCGAGACC TAGAAAAACA TGGAGCAATC | 5580 |
| | ACAAGTAGCA ATACAGCAGC TACCAATGCT GATTGTGCCT GGCTAGAAGC ACAAGAGGAG | 5640 |
| | GAGGAGGTGG GTTTTCCAGT CACACCTCAG GTACCTTTAA GACCAATGAC TTACAAGGCA | 5700 |
| | GCTGTAGATC TTAGCCACTT TTTAAAAGAA AAGGGGGGAC TGGAAGGGCT AATTCACTCC | 5760 |
| | CAACGAAGAC AAGATATCCT TGATCTGTGG ATCTACCACA CACAAGGCTA CTTCCCTGAT | 5820 |
| | TGGCAGAACT ACACACCAGG GCCAGGGATC AGATATCCAC TGACCTTTGG ATGGTGCTAC | 5880 |
| | AAGCTAGTAC CAGTTGAGCA AGAGAAGGTA GAAGAAGCCA ATGAAGGAGA GAACACCCGC | 5940 |
| | TTGTTACACC CTGTGAGCCT GCATGGGATG GATGACCCGG AGAGAGAAGT ATTAGAGTGG | 6000 |
| | AGGTTTGACA GCCGCCTAGC ATTTCATCAC ATGGCCCGAG AGCTGCATCC GGACTGTACT | 6060 |
| | GGGTCTCTCT GGTTAGACCA GATCTGAGCC TGGGAGCTCT CTGGCTAACT AGGGAACCCA | 6120 |
| | CTGCTTAAGC CTCAATAAAG CTTGCCTTGA GTGCTTCAAG TAGTGTGTGC CCGTCTGTTG | 6180 |
| | TGTGACTCTG GTAACTAGAG ATCCCTCAGA CCCTTTTAGT CAGTGTGGAA AATCTCTAGC | 6240 |
| | AGGGCCCGTT TAAACCCGCT GATCAGCCTC GACTGTGCCT TCTAGTTGCC AGCCATCTGT | 6300 |
| | TGTTTGCCCC TCCCCCGTGC CTTCCTTGAC CCTGGAAGGT GCCACTCCCA CTGTCCTTTC | 6360 |
| | CTAATAAAAT GAGGAAATTG CATCGCATTG TCTGAGTAGG TGTCATTCTA TTCTGGGGGG | 6420 |
| | TGGGGTGGGG CAGGACAGCA AGGGGGAGGA TTGGGAAGAC AATAGCAGGC ATGCTGGGGA | 6480 |
| | TGCGGTGGGC TCTATGGCTT CTGAGGCGGA AAGAACCAGC TGGGGCTCTA GGGGGTATCC | 6540 |
| | CCACGCGCCC TGTAGCGGCG CATTAAGCGC GGCGGGTGTG GTGGTTACGC GCAGCGTGAC | 6600 |
| | CGCTACACTT GCCAGCGCCC TAGCGCCCGC TCCTTTCGCT TTCTTCCCTT CCTTTCTCGC | 6660 |
| | CACGTTCGCC GGCTTTCCCC GTCAAGCTCT AAATCGGGGG CTCCCTTTAG GGTTCCGATT | 6720 |
| | TAGTGCTTTA CGGCACCTCG ACCCCAAAAA ACTTGATTAG GGTGATGGTT CACGTAGTGG | 6780 |
| | GCCATCGCCC TGATAGACGG TTTTTCGCCC TTTGACGTTG GAGTCCACGT TCTTTAATAG | 6840 |
| | TGGACTCTTG TTCCAAACTG GAACAACACT CAACCCTATC TCGGTCTATT CTTTTGATTT | 6900 |
| | ATAAGGGATT TTGCCGATTT CGGCCTATTG GTTAAAAAAT GAGCTGATTT AACAAAAATT | 6960 |
| | TAACGCGAAT TAATTCTGTG GAATGTGTGT CAGTTAGGGT GTGGAAAGTC CCCAGGCTCC | 7020 |
| | CCAGCAGGCA GAAGTATGCA AAGCATGCAT CTCAATTAGT CAGCAACCAG GTGTGGAAAG | 7080 |
| | TCCCCAGGCT CCCCAGCAGG CAGAAGTATG CAAAGCATGC ATCTCAATTA GTCAGCAACC | 7140 |
| | ATAGTCCCGC CCCTAACTCC GCCCATCCCG CCCCTAACTC CGCCCAGTTC CGCCCATTCT | 7200 |
| | CCGCCCCATG GCTGACTAAT TTTTTTTATT TATGCAGAGG CCGAGGCCGC CTCTGCCTCT | 7260 |
| | GAGCTATTCC AGAAGTAGTG AGGAGGCTTT TTTGGAGGCC TAGGCTTTTG CAAAAAGCTC | 7320 |
| | CCGGGAGCTT GTATATCCAT TTTCGGATCT GATCAGCACG TGTTGACAAT TAATCATCGG | 7380 |
| | CATAGTATAT CGGCATAGTA TAATACGACA AGGTGAGGAA CTAAACCATG GCCAAGTTGA | 7440 |
| | CCAGTGCCGT TCCGGTGCTC ACCGCGCGCG ACGTCGCCGG AGCGGTCGAG TTCTGGACCG | 7500 |
| | ACCGGCTCGG GTTCTCCCGG GACTTCGTGG AGGACGACTT CGCCGGTGTG GTCCGGGACG | 7560 |
| | ACGTGACCCT GTTCATCAGC GCGGTCCAGG ACCAGGTGGT GCCGGACAAC ACCCTGGCCT | 7620 |
| | GGGTGTGGGT GCGCGGCCTG GACGAGCTGT ACGCCGAGTG GTCGGAGGTC GTGTCCACGA | 7680 |
| | ACTTCCGGGA CGCCECCGGG CCGGCCATGA CCGAGATCGG CGAGCAGCCG TGGGGGCGGG | 7740 |
| | AGTTCGCCCT GCGCGACCCG GCCGGCAACT GCGTGCACTT CGTGGCCGAG GAGCAGGACT | 7800 |
| | GACACGTGCT ACGAGATTTC GATTCCACCG CCGCCTTCTA TGAAAGGTTG GGCTTCGGAA | 7860 |
| | TCGTTTTCCG GGACGCCGGC TGGATGATCC TCCAGCGCGG GGATCTCATG CTGGAGTTCT | 7920 |
| | TCGCCCACCC CAACTTGTTT ATTGCAGCTT ATAATGGTTA CAAATAAAGC AATAGCATCA | 7980 |
| | CAAATTTCAC AAATAAAGCA TTTTTTTCAC TGCATTCTAG TTGTGGTTTG TCCAAACTCA | 8040 |
| | TCAATGTATC TTATCATGTC TGTATACCGT CGACCTCTAG CTAGAGCTTG GCGTAATCAT | 8100 |
| | GGTCATAGCT GTTTCCTGTG TGAAATTGTT ATCCGCTCAC AATTCCACAC AACATACGAG | 8160 |
| | CCGGAAGCAT AAAGTGTAAA GCCTGGGGTG CCTAATGAGT GAGCTAACTC ACATTAATTG | 8220 |
| | CGTTGCGCTC ACTGCCCGCT TTCCAGTCGG GAAACCTGTC GTGCCAGCTG CATTAATGAA | 8280 |
| | TCGGCCAACG CGCGGGGAGA GGCGGTTTGC GTATTGGGCG CTCTTCCGCT TCCTCGCTCA | 8340 |
| | CTGACTCGCT GCGCTCGGTC GTTCGGCTGC GGCGAGCGGT ATCAGCTCAC TCAAAGGCGG | 8400 |
| | TAATACGGTT ATCCACAGAA TCAGGGGATA ACGCAGGAAA GAACATGTGA GCAAAAGGCC | 8460 |
| | AGCAAAAGGC CAGGAACCGT AAAAAGGCCG CGTTGCTGGC GTTTTTCCAT AGGCTCCGCC | 8520 |
| | CCCCTGACGA GCATCACAAA AATCGACGCT CAAGTCAGAG GTGGCGAAAC CCGACAGGAC | 8580 |
| | TATAAAGATA CCAGGCGTTT CCCCCTGGAA GCTCCCTCGT GCGCTCTCCT GTTCCGACCC | 8640 |
| | TGCCGCTTAC CGGATACCTG TCCGCCTTTC TCCCTTCGGG AAGCGTGGCG CTTTCTCATA | 8700 |
| | GCTCACGCTG TAGGTATCTC AGTTCGGTGT AGGTCGTTCG CTCCAAGCTG GGCTGTGTGC | 8760 |
| | ACGAACCCCC CGTTCAGCCC GACCGCTGCG CCTTATCCGG TAACTATCGT CTTGAGTCCA | 8820 |
| | ACCCGGTAAG ACACGACTTA TCGCCACTGG CAGCAGCCAC TGGTAACAGG ATTAGCAGAG | 8880 |
| | CGAGGTATGT AGGCGGTGCT ACAGAGTTCT TGAAGTGGTG GCCTAACTAC GGCTACACTA | 8940 |
| | GAAGAACAGT ATTTGGTATC TGCGCTCTGC TGAAGCCAGT TACCTTCGGA AAAAGAGTTG | 9000 |
| | GTAGCTCTTG ATCCGGCAAA CAAACCACCG CTGGTAGCGG TGGTTTTTTT GTTTGCAAGC | 9060 |
| | AGCAGATTAC GCGCAGAAAA AAAGGATCTC AAGAAGATCC TTTGATCTTT TCTACGGGGT | 9120 |
| | CTGACGCTCA GTGGAACGAA AACTCACGTT AAGGGATTTT GGTCATGAGA TTATCAAAAA | 9180 |
| | GGATCTTCAC CTAGATCCTT TTAAATTAAA AATGAAGTTT TAAATCAATC TAAAGTATAT | 9240 |
| | ATGAGTAAAC TTGGTCTGAC AGTTACCAAT GCTTAATCAG TGAGGCACCT ATCTCAGCGA | 9300 |
| | TCTGTCTATT TCGTTCATCC ATAGTTGCCT GACTCCCCGT CGTGTAGATA ACTACGATAC | 9360 |
| | GGGAGGGCTT ACCATCTGGC CCCAGTGCTG CAATGATACC GCGAGACCCA CGCTCACCGG | 9420 |
| | CTCCAGATTT ATCAGCAATA AACCAGCCAG CCGGAAGGGC CGAGCGCAGA AGTGGTCCTG | 9480 |
| | CAACTTTATC CGCCECCATC CAGTCTATTA ATTGTTGCCG GGAAGCTAGA GTAAGTAGTT | 9540 |
| | CGCCAGTTAA TAGTTTGCGC AACGTTGTTG CCATTGCTAC AGGCATCGTG GTGTCACGCT | 9600 |
| | CGTCGTTTGG TATGGCTTCA TTCAGCTCCG GTTCCCAACG ATCAAGGCGA GTTACATGAT | 9660 |
| | CCCCCATGTT GTGCAAAAAA GCGGTTAGCT CCTTCGGTCC TCCGATCGTT GTCAGAAGTA | 9720 |
| | AGTTGGCCGC AGTGTTATCA CTCATGGTTA TGGCAGCACT GCATAATTCT CTTACTGTCA | 9780 |
| | TGCCATCCGT AAGATGCTTT TCTGTGACTG GTGAGTACTC AACCAAGTCA TTCTGAGAAT | 9840 |
| | AGTGTATGCG GCGACCGAGT TGCTCTTGCC CGGCGTCAAT ACGGGATAAT ACCGCGCCAC | 9900 |
| | ATAGCAGAAC TTTAAAAGTG CTCATCATTG GAAAACGTTC TTCGGGGCGA AAACTCTCAA | 9960 |
| | GGATCTTACC GCTGETGAGA TCCAGTTCGA TGTAACCCAC TCGTGCACCC AACTGATCTT | 10020 |
| | CAGCATCTTT TACTTTCACC AGCGTTTCTG GGTGAGCAAA AACAGGAAGG CAAAATGCCG | 10080 |
| | CAAAAAAGGG AATAAGGGCG ACACGGAAAT GTTGAATACT CATACTCTTC CTTTTTCAAT | 10140 |
| | ATTATTGAAG CATTTATCAG GGTTATTGTC TCATGAGCGG ATACATATTT GAATGTATTT | 10200 |
| | AGAAAAATAA ACAAATAGGG GTTCCGCGCA CATTTCCCCG AAAAGTGCCA CCTGAC | 10256 |
| SEQ ID NO: 701 | GTCGACGGAT CGGGAGATCT CCCGATCCCC TATGGTGCAC TCTCAGTACA ATCTGCTCTG | 60 |
| CCR15: ch | ATGCCGCATA GTTAAGCCAG TATCTGCTCC CTGCTTGTGT GTTGGAGGTC GCTGAGTAGT | 120 |
| TGFbRII-4-1BB | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGATTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCAEGGACT GGACCTGGAT TCTGTTTCTG GTCGCCGCCG CTACAAGAGT | 2940 |
| | TCACAGCACC ATCCCTCCTC ATGTGCAAAA ATCCGTGAAC AACGACATGA TCGTGACCGA | 3000 |
| | CAATAACGGC GCCGTCAAAT TCCCCCAGCT GTGCAAGTTC TGCGACGTGC GGTTTTCTAC | 3060 |
| | ATGTGATAAC CAGAAGTCCT GCATGAGCAA CTGCAGCATC ACAAGCATCT GCGAGAAACC | 3120 |
| | TCAGGAGGTG TGCGTGGCCG TGTGGCGGAA GAACGACGAG AACATCACCC TGGAAACCGT | 3180 |
| | GTGTCACGAC CCCAAGCTGC CTTACCACGA CTTCATCCTG GAAGATGCCG CCTCTCCAAA | 3240 |
| | GTGCATCATG AAGGAAAAGA AAAAGCCCGG CGAGACCTTC TTCATGTGCT CTTGTTCTAG | 3300 |
| | CGATGAGTGC AATGATAACA TCATTTTCAG CGAGGAATAC AACACCAGCA ATCCCGACCT | 3360 |
| | GCTGCTCGTG ATCTTTCAGC CCGCCCCTGC TAGAGAGCCT GGACACTCCC CTCAGATCAT | 3420 |
| | CAGCTTCTTC CTGGCCCTGA CAAGCACAGC CCTGCTGTTT CTGCTGTTCT TCCTGACCCT | 3480 |
| | GAGATTCAGC GTGGTGAAGC GGGGAAGAAA GAAGCTGCTG TACATCTTCA AGCAGCCTTT | 3540 |
| | CATGCGCCCT GTGCAGACCA CCCAGGAGGA GGACGGCTGC AGCTGCAGAT TCCCAGAGGA | 3600 |
| | AGAGGAAGGC GGCTGTGAAC TGTGAGAATT CCGCCCCCCC CCCCCCCCCC CCTCTCCCTC | 3660 |
| | CCCCCCCCCT AACGTTACTG GCCGAAGCCG CTTGGAATAA GGCCGGTGTG CGTTTGTCTA | 3720 |
| | TATGTTATTT TCCACCATAT TGCCGTCTTT TGGCAATGTG AGGGCCCGGA AACCTGGCCC | 3780 |
| | TGTCTTCTTG ACGAGCATTC CTAGGGGTCT TTCCCCTCTC GCCAAAGGAA TGCAAGGTCT | 3840 |
| | GTTGAATGTC GTGAAGGAAG CAGTTCCTCT GGAAGCTTCT TGAAGACAAA CAACGTCTGT | 3900 |
| | AGCGACCCTT TGCAGGCAGC GGAACCCCCC ACCTGGCGAC AGGTGCCTCT GCGGCCAAAA | 3960 |
| | GCCACGTGTA TAAGATACAC CTGCAAAGGC GGCACAACCC CAGTGCCACG TTGTGAGTTG | 4020 |
| | GATAGTTGTG GAAAGAGTCA AATGGCTCTC CTCAAGCGTA TTCAACAAGG GGCTGAAGGA | 4080 |
| | TGCCCAGAAG GTACCCCATT GTATGGGATC TGATCTGGGG CCTCGGTGCA CATGCTTTAC | 4140 |
| | ATGTGTTTAG TCGAGGTTAA AAAAACGTCT AGGCCCCCCG AACCACGGGG ACGTGGTTTT | 4200 |
| | CCTTTGAAAA ACACGATGAT AAGCTTGATC ACGCGTGCCA CCATGAGCAA GGGCGAGGAG | 4260 |
| | CTGTTCACCG GGGTGGTGCC CATCCTGGTC GAGCTGGACG GCGACGTAAA CGGCCACAAG | 4320 |
| | TTCAGCGTGT CCGGCGAGGG CGAGGGCGAT GCCACCTACG GCAAGCTGAC CCTGAAGTTC | 4380 |
| | ATCTGCACCA CCGGCAAGCT GCCCGTGCCC TGGCCCACCC TCGTGACCAC CCTGACCTAC | 4440 |
| | GGCGTGCAGT GCTTCAGCCG CTACCCCGAC CACATGAAGC AGCACGACTT CTTCAAGTCC | 4500 |
| | GCCATGCCCG AAGGCTACGT CCAGGAGCGC ACCATCTTCT TCAAGGACGA CGGCAACTAC | 4560 |
| | AAGACCCGCG CCGAGGTGAA GTTCGAGGGC GACACCCTGG TGAACCGCAT CGAGCTGAAG | 4620 |
| | GGCATCGACT TCAAGGAGGA CGGCAACATC CTGGGGCACA AGCTGGAGTA CAACTACAAC | 4680 |
| | AGCCACAACG TCTATATCAT GGCCGACAAG CAGAAGAACG GCATCAAGGT GAACTTCAAG | 4740 |
| | ATCCGCCACA ACATCGAGGA CGGCAGCGTG CAGCTCGCCG ACCACTACCA GCAGAACACC | 4800 |
| | CCCATCGGCG ACGGCCCCGT GCTGCTGCCC GACAACCACT ACCTGAGCAC CCAGTCCGCC | 4860 |
| | CTGAGCAAAG ACCCCAACGA GAAGCGCGAT CACATGGTCC TGCTGGAGTT CGTGACCGCC | 4920 |
| | GCCGGGATCA CTCACGGCAT GGACGAGCTG TACAAGTGAG AATTCGATAT CAAGCTTATC | 4980 |
| | GGTAATCAAC CTCTGGATTA CAAAATTTGT GAAAGATTGA CTGGTATTCT TAACTATGTT | 5040 |
| | GCTCCTTTTA CGCTATGTGG ATACGCTGCT TTAATGCCTT TGTATCATGC TATTGCTTCC | 5100 |
| | CGTATGGCTT TCATTTTCTC CTCCTTGTAT AAATCCTGGT TGCTGTCTCT TTATGAGGAG | 5160 |
| | TTGTGGCCCG TTGTCAGGCA ACGTGGCGTG GTGTGCACTG TGTTTGCTGA CGCAACCCCC | 5220 |
| | ACTGGTTGGG GCATEGCCAC CACCTGTCAG CTCCTTTCCG GGACTTTCGC TTTCCCCCTC | 5280 |
| | CCTATTGCCA CGGCGGAACT CATCGCCGCC TGCCTTGCCC GCTGCTGGAC AGGGGCTCGG | 5340 |
| | CTGTTGGGCA CTGACAATTC CGTGGTGTTG TCGGGGAAAT CATCGTCCTT TCCTTGGCTG | 5400 |
| | CTCGCCTGTG TTGCCACCTG GATTCTGCGC GGGACGTCCT TCTGCTACGT CCCTTCGGCC | 5460 |
| | CTCAATCCAG CGGACCTTCC TTCCCGCGGC CTGCTGCCGG CTCTGCGGCC TCTTCCGCGT | 5520 |
| | CTTCGCCTTC GCCCTCAGAC GAGTCGGATC TCCCTTTGGG CCGCCTCCCC GCATCGATAC | 5580 |
| | CGTCGACCTC GAGACCTAGA AAAACATGGA GCAATCACAA GTAGCAATAC AGCAGCTACC | 5640 |
| | AATGCTGATT GTGCCTGGCT AGAAGCACAA GAGGAGGAGG AGGTGGGTTT TCCAGTCACA | 5700 |
| | CCTCAGGTAC CTTTAAGACC AATGACTTAC AAGGCAGCTG TAGATCTTAG CCACTTTTTA | 5760 |
| | AAAGAAAAGG GGGGACTGGA AGGGCTAATT CACTCCCAAC GAAGACAAGA TATCCTTGAT | 5820 |
| | CTGTGGATCT ACCACACACA AGGCTACTTC CCTGATTGGC AGAACTACAC ACCAGGGCCA | 5880 |
| | GGGATCAGAT ATCCACTGAC CTTTGGATGG TGCTACAAGC TAGTACCAGT TGAGCAAGAG | 5940 |
| | AAGGTAGAAG AAGCCAATGA AGGAGAGAAC ACCCGCTTGT TACACCCTGT GAGCCTGCAT | 6000 |
| | GGGATGGATG ACCCGGAGAG AGAAGTATTA GAGTGGAGGT TTGACAGCCG CCTAGCATTT | 6060 |
| | CATCACATGG CCCGAGAGCT GCATCCGGAC TGTACTGGGT CTCTCTGGTT AGACCAGATC | 6120 |
| | TGAGCCTGGG AGCTCTCTGG CTAACTAGGG AACCCACTGC TTAAGCCTCA ATAAAGCTTG | 6180 |
| | CCTTGAGTGC TTCAAGTAGT GTGTGCCCGT CTGTTGTGTG ACTCTGGTAA CTAGAGATCC | 6240 |
| | CTCAGACCCT TTTAGTCAGT GTGGAAAATC TCTAGCAGGG CCCGTTTAAA CCCGCTGATC | 6300 |
| | AGCCTCGACT GTGCCTTCTA GTTGCCAGCC ATCTGTTGTT TGCCCCTCCC CCGTGCCTTC | 6360 |
| | CTTGACCCTG GAAGGTGCCA CTCCCACTGT CCTTTCCTAA TAAAATGAGG AAATTGCATC | 6420 |
| | GCATTGTCTG AGTAGGTGTC ATTCTATTCT GGGGGGTGGG GTGGGGCAGG ACAGCAAGGG | 6480 |
| | GGAGGATTGG GAAGACAATA GCAGGCATGC TGGGGATGCG GTGGGCTCTA TGGCTTCTGA | 6540 |
| | GGCGGAAAGA ACCAGCTGGG GCTCTAGGGG GTATCCCCAC GCGCCCTGTA GCGGCGCATT | 6600 |
| | AAGCGCGGCG GGTGTGGTGG TTACGCGCAG CGTGACCGCT ACACTTGCCA GCGCCCTAGC | 6660 |
| | GCCCGCTCCT TTCGCTTTCT TCCCTTCCTT TCTCGCCACG TTCGCCGGCT TTCCCCGTCA | 6720 |
| | AGCTCTAAAT CGGGGGCTCC CTTTAGGGTT CCGATTTAGT GCTTTACGGC ACCTCGACCC | 6780 |
| | CAAAAAACTT GATTAGGGTG ATGGTTCACG TAGTGGGCCA TCGCCCTGAT AGACGGTTTT | 6840 |
| | TCGCCCTTTG ACGTTGGAGT CCACGTTCTT TAATAGTGGA CTCTTGTTCC AAACTGGAAC | 6900 |
| | AACACTCAAC CCTATCTCGG TCTATTCTTT TGATTTATAA GGGATTTTGC CGATTTCGGC | 6960 |
| | CTATTGGTTA AAAAATGAGC TGATTTAACA AAAATTTAAC GCGAATTAAT TCTGTGGAAT | 7020 |
| | GTGTGTCAGT TAGGGTGTGG AAAGTCCCCA GGCTCCCCAG CAGGCAGAAG TATGCAAAGC | 7080 |
| | ATGCATCTCA ATTAGTCAGC AACCAGGTGT GGAAAGTCCC CAGGCTCCCC AGCAGGCAGA | 7140 |
| | AGTATGCAAA GCATGCATCT CAATTAGTCA GCAACCATAG TCCCGCCCCT AACTCCGCCC | 7200 |
| | ATCCCGCCCC TAACTCCGCC CAGTTCCGCC CATTCTCCGC CCCATGGCTG ACTAATTTTT | 7260 |
| | TTTATTTATG CAGAGGCCGA GGCCGCCTCT GCCTCTGAGC TATTCCAGAA GTAGTGAGGA | 7320 |
| | GGCTTTTTTG GAGGCCTAGG CTTTTGCAAA AAGCTCCCGG GAGCTTGTAT ATCCATTTTC | 7380 |
| | GGATCTGATC AGCACGTGTT GACAATTAAT CATCGGCATA GTATATCGGC ATAGTATAAT | 7440 |
| | ACGACAAGGT GAGGAACTAA ACCATGGCCA AGTTGACCAG TGCCGTTCCG GTGCTCACCG | 7500 |
| | CGCGCGACGT CGCCGGAGCG GTCGAGTTCT GGACCGACCG GCTCGGGTTC TCCCGGGACT | 7560 |
| | TCGTGGAGGA CGACTTCGCC GGTGTGGTCC GGGACGACGT GACCCTGTTC ATCAGCGCGG | 7620 |
| | TCCAGGACCA GGTGGTGCCG GACAACACCC TGGCCTGGGT GTGGGTGCGC GGCCTGGACG | 7680 |
| | AGCTGTACGC CGAGTGGTCG GAGGTCGTGT CCACGAACTT CCGGGACGCC TCCGGGCCGG | 7740 |
| | CCATGACCGA GATCGGCGAG CAGCCGTGGG GGCGGGAGTT CGCCCTGCGC GACCCGGCCG | 7800 |
| | GCAACTGCGT GCACTTCGTG GCCGAGGAGC AGGACTGACA CGTGCTACGA GATTTCGATT | 7860 |
| | CCACCGCCGC CTTCTATGAA AGGTTGGGCT TCGGAATCGT TTTCCGGGAC GCCGGCTGGA | 7920 |
| | TGATCCTCCA GCGCGGGGAT CTCATGCTGG AGTTCTTCGC CCACCCCAAC TTGTTTATTG | 7980 |
| | CAGCTTATAA TGGTTACAAA TAAAGCAATA GCATCACAAA TTTCACAAAT AAAGCATTTT | 8040 |
| | TTTCACTGCA TTCTAGTTGT GGTTTGTCCA AACTCATCAA TGTATCTTAT CATGTCTGTA | 8100 |
| | TACCGTCGAC CTCTAGCTAG AGCTTGGCGT AATCATGGTC ATAGCTGTTT CCTGTGTGAA | 8160 |
| | ATTGTTATCC GCTCACAATT CCACACAACA TACGAGCCGG AAGCATAAAG TGTAAAGCCT | 8220 |
| | GGGGTGCCTA ATGAGTGAGC TAACTCACAT TAATTGCGTT GCGCTCACTG CCCGCTTTCC | 8280 |
| | AGTCGGGAAA CCTGTCGTGC CAGCTGCATT AATGAATCGG CCAACGCGCG GGGAGAGGCG | 8340 |
| | GTTTGCGTAT TGGGCGCTCT TCCGCTTCCT CGCTCACTGA CTCGCTGCGC TCGGTCGTTC | 8400 |
| | GGCTGCGGCG AGCGGTATCA GCTCACTCAA AGGCGGTAAT ACGGTTATCC ACAGAATCAG | 8460 |
| | GGGATAACGC AGGAAAGAAC ATGTGAGCAA AAGGCCAGCA AAAGGCCAGG AACCGTAAAA | 8520 |
| | AGGCCGCGTT GCTGGCGTTT TTCCATAGGC TCCGCCCCCC TGACGAGCAT CACAAAAATC | 8580 |
| | GACGCTCAAG TCAGAGGTGG CGAAACCCGA CAGGACTATA AAGATACCAG GCGTTTCCCC | 8640 |
| | CTGGAAGCTC CCTCGTGCGC TCTCCTGTTC CGACCCTGCC GCTTACCGGA TACCTGTCCG | 8700 |
| | CCTTTCTCCC TTCGGGAAGC GTGGCGCTTT CTCATAGCTC ACGCTGTAGG TATCTCAGTT | 8760 |
| | CGGTGTAGGT CGTTCGCTCC AAGCTGGGCT GTGTGCACGA ACCCCCCGTT CAGCCCGACC | 8820 |
| | GCTGCGCCTT ATCCGGTAAC TATCGTCTTG AGTCCAACCC GGTAAGACAC GACTTATCGC | 8880 |
| | CACTGGCAGC AGCCACTGGT AACAGGATTA GCAGAGCGAG GTATGTAGGC GGTGCTACAG | 8940 |
| | AGTTCTTGAA GTGGTGGCCT AACTACGGCT ACACTAGAAG AACAGTATTT GGTATCTGCG | 9000 |
| | CTCTGCTGAA GCCAGTTACC TTCGGAAAAA GAGTTGGTAG CTCTTGATCC GGCAAACAAA | 9060 |
| | CCACCGCTGG TAGCGGTGGT TTTTTTGTTT GCAAGCAGCA GATTACGCGC AGAAAAAAAG | 9120 |
| | GATCTCAAGA AGATCCTTTG ATCTTTTCTA CGGGGTCTGA CGCTCAGTGG AACGAAAACT | 9180 |
| | CACGTTAAGG GATTTTGGTC ATGAGATTAT CAAAAAGGAT CTTCACCTAG ATCCTTTTAA | 9240 |
| | ATTAAAAATG AAGTTTTAAA TCAATCTAAA GTATATATGA GTAAACTTGG TCTGACAGTT | 9300 |
| | ACCAATGCTT AATCAGTGAG GCACCTATCT CAGCGATCTG TCTATTTCGT TCATCCATAG | 9360 |
| | TTGCCTGACT CCCCGTCGTG TAGATAACTA CGATACGGGA GGGCTTACCA TCTGGCCCCA | 9420 |
| | GTGCTGCAAT GATACCGCGA GACCCACGCT CACCGGCTCC AGATTTATCA GCAATAAACC | 9480 |
| | AGCCAGCCGG AAGGGCCGAG CGCAGAAGTG GTCCTGCAAC TTTATCCGCC TCCATCCAGT | 9540 |
| | CTATTAATTG TTGCCGGGAA GCTAGAGTAA GTAGTTCGCC AGTTAATAGT TTGCGCAACG | 9600 |
| | TTGTTGCCAT TGCTACAGGC ATCGTGGTGT CACGCTCGTC GTTTGGTATG GCTTCATTCA | 9660 |
| | GCTCCGGTTC CCAACGATCA AGGCGAGTTA CATGATCCCC CATGTTGTGC AAAAAAGCGG | 9720 |
| | TTAGCTCCTT CGGTCCTCCG ATCGTTGTCA GAAGTAAGTT GGCCGCAGTG TTATCACTCA | 9780 |
| | TGGTTATGGC AGCACTGCAT AATTCTCTTA CTGTCATGCC ATCCGTAAGA TGCTTTTCTG | 9840 |
| | TGACTGGTGA GTACTCAACC AAGTCATTCT GAGAATAGTG TATGCGGCGA CCGAGTTGCT | 9900 |
| | CTTGCCCGGC GTCAATACGG GATAATACCG CGCCACATAG CAGAACTTTA AAAGTGCTCA | 9960 |
| | TCATTGGAAA ACGTTCTTCG GGGCGAAAAC TCTCAAGGAT CTTACCGCTG TTGAGATCCA | 10020 |
| | GTTCGATGTA ACCCACTCGT GCACCCAACT GATCTTCAGC ATCTTTTACT TTCACCAGCG | 10080 |
| | TTTCTGGGTG AGCAAAAACA GGAAGGCAAA ATGCCGCAAA AAAGGGAATA AGGGCGACAC | 10140 |
| | GGAAATGTTG AATACTCATA CTCTTCCTTT TTCAATATTA TTGAAGCATT TATCAGGGTT | 10200 |
| | ATTGTCTCAT GAGCGGATAC ATATTTGAAT GTATTTAGAA AAATAAACAA ATAGGGGTTC | 10260 |
| | CGCGCACATT TCCCCGAAAA GTGCCACCTG AC | 10292 |
| SEQ ID NO: 702 | GTCGACGGAT CGGGAGATCT CCCGATCCCC TATGGTGCAC TCTCAGTACA ATCTGCTCTG | 60 |
| CCR16: ch PD-1- | ATGCCGCATA GTTAAGCCAG TATCTGCTCC CTGCTTGTGT GTTGGAGGTC GCTGAGTAGT | 120 |
| CD28 | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGASTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCAEGGACT GGACCTGGAT TCTGTTTCTG GTGGCCGCCG CTACAAGAGT | 2940 |
| | GCACAGCTTC CTGGATTCTC CAGACCGGCC TTGGAACCCC CCCACCTTCT CCCCAGCCCT | 3000 |
| | GCTGGTGGTG ACAGAGGGCG ACAACGCCAC CTTCACATGC AGCTTTTCTA ATACCAGCGA | 3060 |
| | GAGCTTCGTG CTGAATTGGT ACAGAATGTC CCCTAGCAAC CAGACCGACA AGCTGGCCGC | 3120 |
| | TTTTCCTGAG GACAGATCTC AGCCTGGACA GGATTGCAGA TTCAGAGTGA CCCAGCTGCC | 3180 |
| | TAACGGCAGA GACTTCCACA TGAGCGTGGT CAGAGCCAGA CGGAACGACA GCGGAACATA | 3240 |
| | TCTGTGCGGC GCCASCAGCC TGGCCCCTAA GGCCCAAATC AAGGAAAGCC TGAGAGCTGA | 3300 |
| | ACTGAGGGTT ACCGAGCGGC GGGCCGAAGT GCCCACAGCC CACTGCCCTA GCCCTCTGTT | 3360 |
| | CCCCGGCCCC AGCAAACCTT TCTGGGTCCT GGTGGTGGTG GGCGGCGTGC TGGCTTGTTA | 3420 |
| | CAGCCTCCTG GTGACCGTGG CCTTCATCAT CTTCTGGGTG CGGAGCAAGC GGAGTAGACT | 3480 |
| | GCTGCATTCT GATTACATGA ACATGACCCC TAGACGGCCT GGCCCTACCA GAAAGCACTA | 3540 |
| | CCAGCCCTAC GCCCCTCCAA GAGATTTCGC CGCCTACCGC TCCTGAGAAT TCCGCCCCCC | 3600 |
| | CCCCCCCCCC CCCTCTCCCT CCCCCCCCCC TAACGTTACT GGCCGAAGCC GCTTGGAATA | 3660 |
| | AGGCCGGTGT GCGTETGTCT ATATGTTATT TTCCACCATA TTGCCGTCTT TTGGCAATGT | 3720 |
| | GAGGGCCCGG AAACCTGGCC CTGTCTTCTT GACGAGCATT CCTAGGGGTC TTTCCCCTCT | 3780 |
| | CGCCAAAGGA ATGCAAGGTC TGTTGAATGT CGTGAAGGAA GCAGTTCCTC TGGAAGCTTC | 3840 |
| | TTGAAGACAA ACAACGTCTG TAGCGACCCT TTGCAGGCAG CGGAACCCCC CACCTGGCGA | 3900 |
| | CAGGTGCCTC TGCGGCCAAA AGCCACGTGT ATAAGATACA CCTGCAAAGG CGGCACAACC | 3960 |
| | CCAGTGCCAC GTTGTGAGTT GGATAGTTGT GGAAAGAGTC AAATGGCTCT CCTCAAGCGT | 4020 |
| | ATTCAACAAG GGGCTGAAGG ATGCCCAGAA GGTACCCCAT TGTATGGGAT CTGATCTGGG | 4080 |
| | GCCTCGGTGC ACATGCTTTA CATGTGTTTA GTCGAGGTTA AAAAAACGTC TAGGCCCCCC | 4140 |
| | GAACCACGGG GACGTGGTTT TCCTTTGAAA AACACGATGA TAAGCTTGAT CACGCGTGCC | 4200 |
| | ACCATGAGCA AGGGCGAGGA GCTGTTCACC GGGGTGGTGC CCATCCTGGT CGAGCTGGAC | 4260 |
| | GGCGACGTAA ACGGCCACAA GTTCAGCGTG TCCGGCGAGG GCGAGGGCGA TGCCACCTAC | 4320 |
| | GGCAAGCTGA CCCTGAAGTT CATCTGCACC ACCGGCAAGC TGCCCGTGCC CTGGCCCACC | 4380 |
| | CTCGTGACCA CCCTGACCTA CGGCGTGCAG TGCTTCAGCC GCTACCCCGA CCACATGAAG | 4440 |
| | CAGCACGACT TCTTCAAGTC CGCCATGCCC GAAGGCTACG TCCAGGAGCG CACCATCTTC | 4500 |
| | TTCAAGGACG ACGGCAACTA CAAGACCCGC GCCGAGGTGA AGTTCGAGGG CGACACCCTG | 4560 |
| | GTGAACCGCA TCGAGCTGAA GGGCATCGAC TTCAAGGAGG ACGGCAACAT CCTGGGGCAC | 4620 |
| | AAGCTGGAGT ACAACTACAA CAGCCACAAC GTCTATATCA TGGCCGACAA GCAGAAGAAC | 4680 |
| | GGCATCAAGG TGAACTTCAA GATCCGCCAC AACATCGAGG ACGGCAGCGT GCAGCTCGCC | 4740 |
| | GACCACTACC AGCAGAACAC CCCCATCGGC GACGGCCCCG TGCTGCTGCC CGACAACCAC | 4800 |
| | TACCTGAGCA CCCAGTCCGC CCTGAGCAAA GACCCCAACG AGAAGCGCGA TCACATGGTC | 4860 |
| | CTGCTGGAGT TCGTGACCGC CGCCGGGATC ACTCACGGCA TGGACGAGCT GTACAAGTGA | 4920 |
| | GAATTCGATA TCAAGCTTAT CGGTAATCAA CCTCTGGATT ACAAAATTTG TGAAAGATTG | 4980 |
| | ACTGGTATTC TTAACTATGT TGCTCCTTTT ACGCTATGTG GATACGCTGC TTTAATGCCT | 5040 |
| | TTGTATCATG CTATTGCTTC CCGTATGGCT TTCATTTTCT CCTCCTTGTA TAAATCCTGG | 5100 |
| | TTGCTGTCTC TTTATGAGGA GTTGTGGCCC GTTGTCAGGC AACGTGGCGT GGTGTGCACT | 5160 |
| | GTGTTTGCTG ACGCAACCCC CACTGGTTGG GGCATTGCCA CCACCTGTCA GCTCCTTTCC | 5220 |
| | GGGACTTTCG CTTTCCCCCT CCCTATTGCC ACGGCGGAAC TCATCGCCGC CTGCCTTGCC | 5280 |
| | CGCTGCTGGA CAGGGGCTCG GCTGTTGGGC ACTGACAATT CCGTGGTGTT GTCGGGGAAA | 5340 |
| | TCATCGTCCT TTCCTTGGCT GCTCGCCTGT GTTGCCACCT GGATTCTGCG CGGGACGTCC | 5400 |
| | TTCTGCTACG TCCCTTCGGC CCTCAATCCA GCGGACCTTC CTTCCCGCGG CCTGCTGCCG | 5460 |
| | GCTCTGCGGC CTCTTCCGCG TCTTCGCCTT CGCCCTCAGA CGAGTCGGAT CTCCCTTTGG | 5520 |
| | GCCGCCTCCC CGCATCGATA CCGTCGACCT CGAGACCTAG AAAAACATGG AGCAATCACA | 5580 |
| | AGTAGCAATA CAGCAGCTAC CAATGCTGAT TGTGCCTGGC TAGAAGCACA AGAGGAGGAG | 5640 |
| | GAGGTGGGTT TTCCAGTCAC ACCTCAGGTA CCTTTAAGAC CAATGACTTA CAAGGCAGCT | 5700 |
| | GTAGATCTTA GCCACTTTTT AAAAGAAAAG GGGGGACTGG AAGGGCTAAT TCACTCCCAA | 5760 |
| | CGAAGACAAG ATATCCTTGA TCTGTGGATC TACCACACAC AAGGCTACTT CCCTGATTGG | 5820 |
| | CAGAACTACA CACCAGGGCC AGGGATCAGA TATCCACTGA CCTTTGGATG GTGCTACAAG | 5880 |
| | CTAGTACCAG TTGAGCAAGA GAAGGTAGAA GAAGCCAATG AAGGAGAGAA CACCCGCTTG | 5940 |
| | TTACACCCTG TGAGCCTGCA TGGGATGGAT GACCCGGAGA GAGAAGTATT AGAGTGGAGG | 6000 |
| | TTTGACAGCC GCCTAGCATT TCATCACATG GCCCGAGAGC TGCATCCGGA CTGTACTGGG | 6060 |
| | TCTCTCTGGT TAGACCAGAT CTGAGCCTGG GAGCTCTCTG GCTAACTAGG GAACCCACTG | 6120 |
| | CTTAAGCCTC AATAAAGCTT GCCTTGAGTG CTTCAAGTAG TGTGTGCCCG TCTGTTGTGT | 6180 |
| | GACTCTGGTA ACTAGAGATC CCTCAGACCC TTTTAGTCAG TGTGGAAAAT CTCTAGCAGG | 6240 |
| | GCCCGTTTAA ACCCGCTGAT CAGCCTCGAC TGTGCCTTCT AGTTGCCAGC CATCTGTTGT | 6300 |
| | TTGCCCCTCC CCCGTGCCTT CCTTGACCCT GGAAGGTGCC ACTCCCACTG TCCTTTCCTA | 6360 |
| | ATAAAATGAG GAAATTGCAT CGCATTGTCT GAGTAGGTGT CATTCTATTC TGGGGGGTGG | 6420 |
| | GGTGGGGCAG GACAGCAAGG GGGAGGATTG GGAAGACAAT AGCAGGCATG CTGGGGATGC | 6480 |
| | GGTGGGCTCT ATGGCTTCTG AGGCGGAAAG AACCAGCTGG GGCTCTAGGG GGTATCCCCA | 6540 |
| | CGCGCCCTGT AGCGGCGCAT TAAGCGCGGC GGGTGTGGTG GTTACGCGCA GCGTGACCGC | 6600 |
| | TACACTTGCC AGCGCCCTAG CGCCCGCTCC TTTCGCTTTC TTCCCTTCCT TTCTCGCCAC | 6660 |
| | GTTCGCCGGC TTTCCCCGTC AAGCTCTAAA TCGGGGGCTC CCTTTAGGGT TCCGATTTAG | 6720 |
| | TGCTTTACGG CACCTCGACC CCAAAAAACT TGATTAGGGT GATGGTTCAC GTAGTGGGCC | 6780 |
| | ATCGCCCTGA TAGACGGTTT TTCGCCCTTT GACGTTGGAG TCCACGTTCT TTAATAGTGG | 6840 |
| | ACTCTTGTTC CAAACTGGAA CAACACTCAA CCCTATCTCG GTCTATTCTT TTGATTTATA | 6900 |
| | AGGGATTTTG CCGASTTCGG CCTATTGGTT AAAAAATGAG CTGATTTAAC AAAAATTTAA | 6960 |
| | CGCGAATTAA TTCTGTGGAA TGTGTGTCAG TTAGGGTGTG GAAAGTCCCC AGGCTCCCCA | 7020 |
| | GCAGGCAGAA GTATGCAAAG CATGCATCTC AATTAGTCAG CAACCAGGTG TGGAAAGTCC | 7080 |
| | CCAGGCTCCC CAGCAGGCAG AAGTATGCAA AGCATGCATC TCAATTAGTC AGCAACCATA | 7140 |
| | GTCCCGCCCC TAACTCCGCC CATCCCGCCC CTAACTCCGC CCAGTTCCGC CCATTCTCCG | 7200 |
| | CCCCATGGCT GACTAATTTT TTTTATTTAT GCAGAGGCCG AGGCCGCCTC TGCCTCTGAG | 7260 |
| | CTATTCCAGA AGTAGTGAGG AGGCTTTTTT GGAGGCCTAG GCTTTTGCAA AAAGCTCCCG | 7320 |
| | GGAGCTTGTA TATCCATTTT CGGATCTGAT CAGCACGTGT TGACAATTAA TCATCGGCAT | 7380 |
| | AGTATATCGG CATAGTATAA TACGACAAGG TGAGGAACTA AACCATGGCC AAGTTGACCA | 7440 |
| | GTGCCGTTCC GGTGCTCACC GCGCGCGACG TCGCCGGAGC GGTCGAGTTC TGGACCGACC | 7500 |
| | GGCTCGGGTT CTCCCGGGAC TTCGTGGAGG ACGACTTCGC CGGTGTGGTC CGGGACGACG | 7560 |
| | TGACCCTGTT CATCAGCGCG GTCCAGGACC AGGTGGTGCC GGACAACACC CTGGCCTGGG | 7620 |
| | TGTGGGTGCG CGGCCTGGAC GAGCTGTACG CCGAGTGGTC GGAGGTCGTG TCCACGAACT | 7680 |
| | TCCGGGACGC CTCCGGGCCG GCCATGACCG AGATCGGCGA GCAGCCGTGG GGGCGGGAGT | 7740 |
| | TCGCCCTGCG CGACCCGGCC GGCAACTGCG TGCACTTCGT GGCCGAGGAG CAGGACTGAC | 7800 |
| | ACGTGCTACG AGATTTCGAT TCCACCGCCG CCTTCTATGA AAGGTTGGGC TTCGGAATCG | 7860 |
| | TTTTCCGGGA CGCCGGCTGG ATGATCCTCC AGCGCGGGGA TCTCATGCTG GAGTTCTTCG | 7920 |
| | CCCACCCCAA CTTGETTATT GCAGCTTATA ATGGTTACAA ATAAAGCAAT AGCATCACAA | 7980 |
| | ATTTCACAAA TAAAGCATTT TTTTCACTGC ATTCTAGTTG TGGTTTGTCC AAACTCATCA | 8040 |
| | ATGTATCTTA TCATGTCTGT ATACCGTCGA CCTCTAGCTA GAGCTTGGCG TAATCATGGT | 8100 |
| | CATAGCTGTT TCCTGTGTGA AATTGTTATC CGCTCACAAT TCCACACAAC ATACGAGCCG | 8160 |
| | GAAGCATAAA GTGTAAAGCC TGGGGTGCCT AATGAGTGAG CTAACTCACA TTAATTGCGT | 8220 |
| | TGCGCTCACT GCCCGCTTTC CAGTCGGGAA ACCTGTCGTG CCAGCTGCAT TAATGAATCG | 8280 |
| | GCCAACGCGC GGGGAGAGGC GGTTTGCGTA TTGGGCGCTC TTCCGCTTCC TCGCTCACTG | 8340 |
| | ACTCGCTGCG CTCGGTCGTT CGGCTGCGGC GAGCGGTATC AGCTCACTCA AAGGCGGTAA | 8400 |
| | TACGGTTATC CACAGAATCA GGGGATAACG CAGGAAAGAA CATGTGAGCA AAAGGCCAGC | 8460 |
| | AAAAGGCCAG GAACCGTAAA AAGGCCGCGT TGCTGGCGTT TTTCCATAGG CTCCGCCCCC | 8520 |
| | CTGACGAGCA TCACAAAAAT CGACGCTCAA GTCAGAGGTG GCGAAACCCG ACAGGACTAT | 8580 |
| | AAAGATACCA GGCGTTTCCC CCTGGAAGCT CCCTCGTGCG CTCTCCTGTT CCGACCCTGC | 8640 |
| | CGCTTACCGG ATACCTGTCC GCCTTTCTCC CTTCGGGAAG CGTGGCGCTT TCTCATAGCT | 8700 |
| | CACGCTGTAG GTATCTCAGT TCGGTGTAGG TCGTTCGCTC CAAGCTGGGC TGTGTGCACG | 8760 |
| | AACCCCCCGT TCAGCCCGAC CGCTGCGCCT TATCCGGTAA CTATCGTCTT GAGTCCAACC | 8820 |
| | CGGTAAGACA CGACTTATCG CCACTGGCAG CAGCCACTGG TAACAGGATT AGCAGAGCGA | 8880 |
| | GGTATGTAGG CGGTGCTACA GAGTTCTTGA AGTGGTGGCC TAACTACGGC TACACTAGAA | 8940 |
| | GAACAGTATT TGGTATCTGC GCTCTGCTGA AGCCAGTTAC CTTCGGAAAA AGAGTTGGTA | 9000 |
| | GCTCTTGATC CGGCAAACAA ACCACCGCTG GTAGCGGTGG TTTTTTTGTT TGCAAGCAGC | 9060 |
| | AGATTACGCG CAGAAAAAAA GGATCTCAAG AAGATCCTTT GATCTTTTCT ACGGGGTCTG | 9120 |
| | ACGCTCAGTG GAACGAAAAC TCACGTTAAG GGATTTTGGT CATGAGATTA TCAAAAAGGA | 9180 |
| | TCTTCACCTA GATCCTTTTA AATTAAAAAT GAAGTTTTAA ATCAATCTAA AGTATATATG | 9240 |
| | AGTAAACTTG GTCTGACAGT TACCAATGCT TAATCAGTGA GGCACCTATC TCAGCGATCT | 9300 |
| | GTCTATTTCG TTCASCCATA GTTGCCTGAC TCCCCGTCGT GTAGATAACT ACGATACGGG | 9360 |
| | AGGGCTTACC ATCTGGCCCC AGTGCTGCAA TGATACCGCG AGACCCACGC TCACCGGCTC | 9420 |
| | CAGATTTATC AGCAATAAAC CAGCCAGCCG GAAGGGCCGA GCGCAGAAGT GGTCCTGCAA | 9480 |
| | CTTTATCCGC CTCCATCCAG TCTATTAATT GTTGCCGGGA AGCTAGAGTA AGTAGTTCGC | 9540 |
| | CAGTTAATAG TTTGCGCAAC GTTGTTGCCA TTGCTACAGG CATCGTGGTG TCACGCTCGT | 9600 |
| | CGTTTGGTAT GGCTTCATTC AGCTCCGGTT CCCAACGATC AAGGCGAGTT ACATGATCCC | 9660 |
| | CCATGTTGTG CAAAAAAGCG GTTAGCTCCT TCGGTCCTCC GATCGTTGTC AGAAGTAAGT | 9720 |
| | TGGCCGCAGT GTTATCACTC ATGGTTATGG CAGCACTGCA TAATTCTCTT ACTGTCATGC | 9780 |
| | CATCCGTAAG ATGCTTTTCT GTGACTGGTG AGTACTCAAC CAAGTCATTC TGAGAATAGT | 9840 |
| | GTATGCGGCG ACCGAGTTGC TCTTGCCCGG CGTCAATACG GGATAATACC GCGCCACATA | 9900 |
| | GCAGAACTTT AAAAGTGCTC ATCATTGGAA AACGTTCTTC GGGGCGAAAA CTCTCAAGGA | 9960 |
| | TCTTACCGCT GTTGAGATCC AGTTCGATGT AACCCACTCG TGCACCCAAC TGATCTTCAG | 10020 |
| | CATCTTTTAC TTTCACCAGC GTTTCTGGGT GAGCAAAAAC AGGAAGGCAA AATGCCGCAA | 10080 |
| | AAAAGGGAAT AAGGGCGACA CGGAAATGTT GAATACTCAT ACTCTTCCTT TTTCAATATT | 10140 |
| | ATTGAAGCAT TTATCAGGGT TATTGTCTCA TGAGCGGATA CATATTTGAA TGTATTTAGA | 10200 |
| | AAAATAAACA AATAGGGGTT CCGCGCACAT TTCCCCGAAA AGTGCCACCT GAC | 10253 |

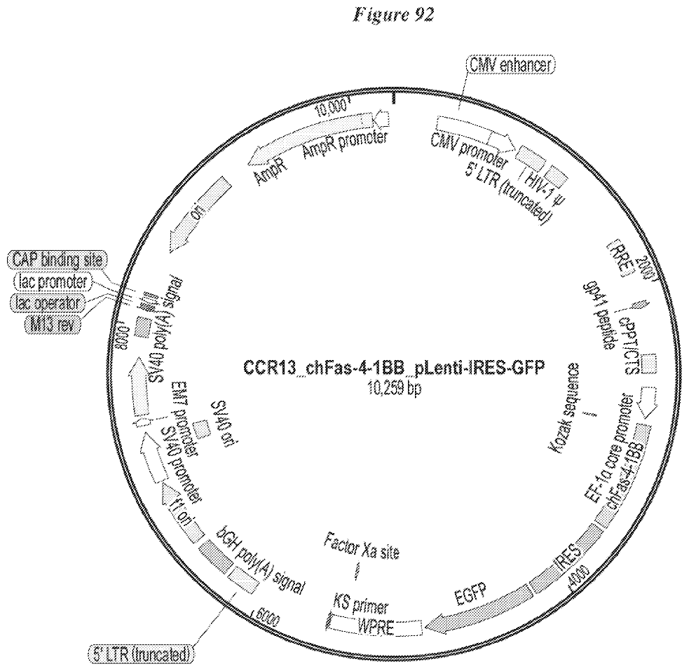

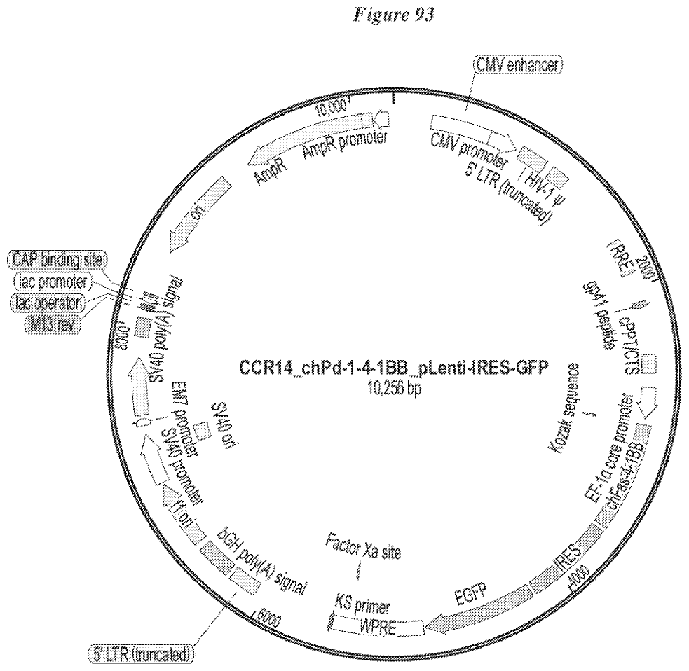

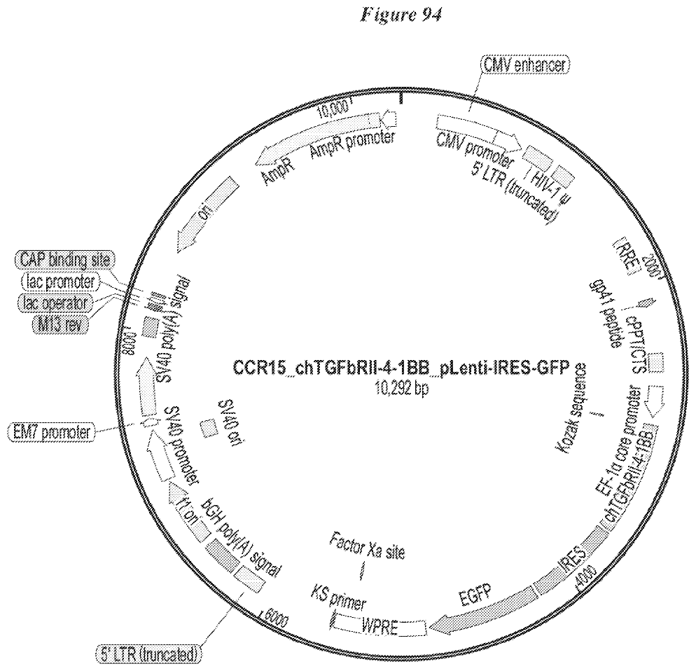

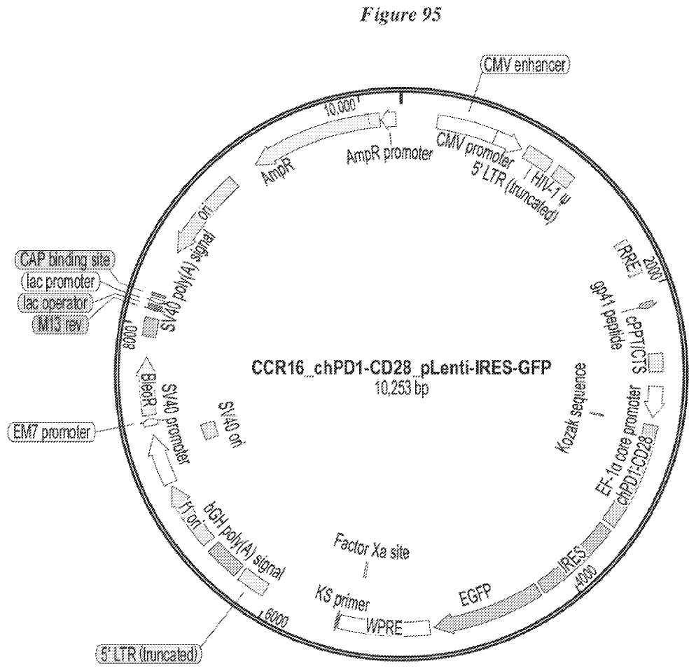

Vector maps for the exemplary vectors encoding the CCRs designated CCR13, CCR14, CCR15, and CCR16, corresponding to SEQ ID NO:699 to SEQ ID NO:702, are presented in FIGS. 92 to 95. These vectors were used for the preparation of the lentiviral batches in this example.

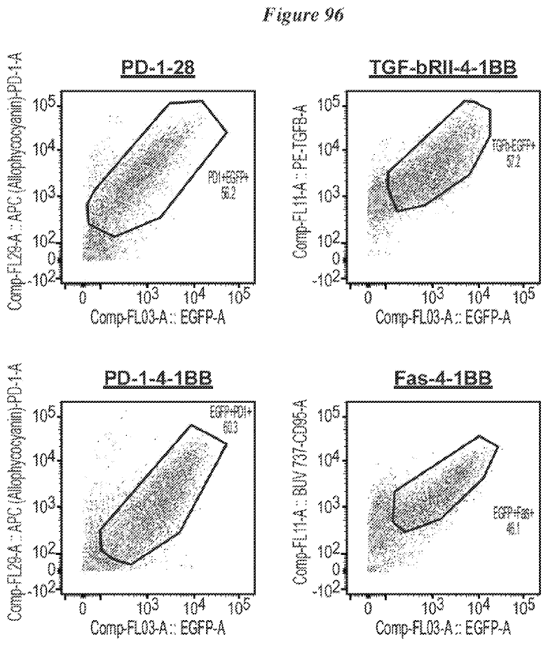

After preparation of the lentivirus, TILs were transduced using each lentivirus batch, followed by resting 2 days, then followed with an 11-day REP expansion process. Surface expression of each CCR construct was detected by flow cytometry. Results are shown in FIG. 96.

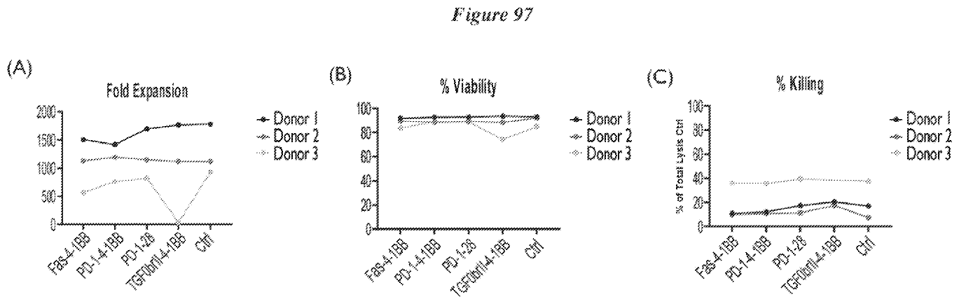

Expansion, viability and killing efficacy of REP TILs expressing CCRs was also assessed. Pre-REP TILs (N=3) were activated with Trans-ACT for 2 days, followed with gene transduction with lentiviral particles containing CCR construct including Fas-4-1BB, PD-1-4-1BB, TGF-bRII-4-1BB, PD-1-28, (i.e., CCR13, CCR14, CCR15, and CCR16) and control vehicle vector. Two days after gene transduction, 3×10⁴ pre-REP TILs were processed with an 11-day REP expansion. FIG. 97(A) shows fold expansion of CCR-expressing post-REP TILs, (B) shows viability, and (C) shows killing capability of CCR-expressing post-REP TILs evaluated by the KILR® cytotoxicity assay. Briefly, KILR® THP-1 target cells (1.25×10⁴) were co-cultured with CCR transduced TIL cells (1.25×10⁵, E:T ratio=10:1) in 96 well white plates with 100 μL CM2 culture medium containing 300 IU/mL IL-2. After 24 hours, 100 μL of KILR reagent was added into each well, followed with 30 mins incubation. Luminescence signal was determined to quantify dead cells. The percentage of killing was normalized based on control wells, which were added with cell lysis buffer.

The foregoing examples are also embodiments of the present invention. Further embodiments of present invention include the sequences of SEQ ID NO:689, SEQ ID NO:690, SEQ ID NO:691, SEQ ID NO:692, SEQ ID NO:693, and SEQ ID NO:694, or conservative amino acid substitutions thereof, or fragments, variants, or derivatives thereof, or an amino acid sequence that is at least 99% identical to the sequence given in SEQ ID NO:689, SEQ ID NO:690, SEQ ID NO:691, SEQ ID NO:692, SEQ ID NO:693, and SEQ ID NO:694, at least 98% identical to the sequence given in SEQ ID NO:689, SEQ ID NO:690, SEQ ID NO:691, SEQ ID NO:692, SEQ ID NO:693, and SEQ ID NO:694, at least 97% identical to the sequence given in SEQ ID NO:689, SEQ ID NO:690, SEQ ID NO:691, SEQ ID NO:692, SEQ ID NO:693, and SEQ ID NO:694, at least 96% identical to the sequence given in SEQ ID NO:689, SEQ ID NO:690, SEQ ID NO:691, SEQ ID NO:692, SEQ ID NO:693, and SEQ ID NO:694, at least 95% identical to the sequence given in SEQ ID NO:689, SEQ ID NO:690, SEQ ID NO:691, SEQ ID NO:692, SEQ ID NO:693, and SEQ ID NO:694, at least 90% identical to the sequence given in SEQ ID NO:689, SEQ ID NO:690, SEQ ID NO:691, SEQ ID NO:692, SEQ ID NO:693, and SEQ ID NO:694 at least 85% identical to the sequence given in SEQ ID NO:689, SEQ ID NO:690, SEQ ID NO:691, SEQ ID NO:692, SEQ ID NO:693, and SEQ ID NO:694, or at least 80% identical to the sequence given in SEQ ID NO:689, SEQ ID NO:690, SEQ ID NO:691, SEQ ID NO:692, SEQ ID NO:693, and SEQ ID NO:694.

Further embodiments of present invention include the sequences of SEQ ID NO:695, SEQ ID NO:696, SEQ ID NO:697, and SEQ ID NO:698, or fragments, variants, or derivatives thereof, or a nucleotide sequence that is at least 99% identical to the sequence given in SEQ ID NO:695, SEQ ID NO:696, SEQ ID NO:697, and SEQ ID NO:698, at least 98% identical to the sequence given in SEQ ID NO:695, SEQ ID NO:696, SEQ ID NO:697, and SEQ ID NO:698, at least 97% identical to the sequence given in SEQ ID NO:695, SEQ ID NO:696, SEQ ID NO:697, and SEQ ID NO:698, at least 96% identical to the sequence given in SEQ ID NO:695, SEQ ID NO:696, SEQ ID NO:697, and SEQ ID NO:698, at least 95% identical to the sequence given in SEQ ID NO:695, SEQ ID NO:696, SEQ ID NO:697, and SEQ ID NO:698, at least 90% identical to the sequence given in SEQ ID NO:695, SEQ ID NO:696, SEQ ID NO:697, and SEQ ID NO:698, at least 85% identical to the sequence given in SEQ ID NO:695, SEQ ID NO:696, SEQ ID NO:697, and SEQ ID NO:698, or at least 80% identical to the sequence given in SEQ ID NO:695, SEQ ID NO:696, SEQ ID NO:697, and SEQ ID NO:698.

Further embodiments of present invention include the sequences of SEQ ID NO:699, SEQ ID NO:700, SEQ ID NO:701, and SEQ ID NO:702, or fragments, variants, or derivatives thereof, or a nucleotide sequence that is at least 99% identical to the sequence given in SEQ ID NO:699, SEQ ID NO:700, SEQ ID NO:701, and SEQ ID NO:702, at least 98% identical to the sequence given in SEQ ID NO:699, SEQ ID NO:700, SEQ ID NO:701, and SEQ ID NO:702, at least 97% identical to the sequence given in SEQ ID NO:699, SEQ ID NO:700, SEQ ID NO:701, and SEQ ID NO:702, at least 96% identical to the sequence given in SEQ ID NO:699, SEQ ID NO:700, SEQ ID NO:701, and SEQ ID NO:702, at least 95% identical to the sequence given in SEQ ID NO:699, SEQ ID NO:700, SEQ ID NO:701, and SEQ ID NO:702, at least 90% identical to the sequence given in SEQ ID NO:699, SEQ ID NO:700, SEQ ID NO:701, and SEQ ID NO:702, at least 85% identical to the sequence given in SEQ ID NO:699, SEQ ID NO:700, SEQ ID NO:701, and SEQ ID NO:702, or at least 80% identical to the sequence given in SEQ ID NO:699, SEQ ID NO:700, SEQ ID NO:701, and SEQ ID NO:702.

### Example 25: Chimeric Costimulatory Receptors with LTBR Intracellular Domains

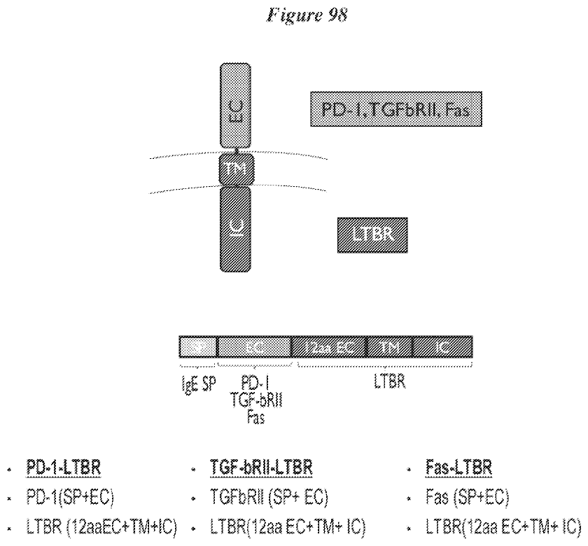

Using the methods described above, additional CCRs may be prepared. FIG. 98 shows constructs for CCRs designated CCR17, CCR18, and CCR19, using the LTBR intracellular domain. Amino acid sequences for these CCRs are set forth in Table 93.

TABLE 93
Amino acid sequences of exemplary CCRs designated CCR17, CCR18, and
CCR19.

| Identifier | Sequence (One-Letter Amino Acid Symbols) | |
|---|---|---|
| SEQ ID NO: 703 | MDWTWILFLV AAATRVHSQV TDINSKGLEL RKTVTTVETQ NLEGLHHDGQ FCHKPCPPGE | 60 |
| CCR17: ch Fas- | RKARDCTVNG DEPDCVPCQE GKEYTDKAHF SSKCRRCRLC DEGHGLEVEI NCTRTQNTKC | 120 |
| LTBR | RCKPNFFCNS TVCEHCDPCT KCEHGIIKEC TLTSNTKCKE EGSRSNPLPP EMSGTMLMLA | 180 |
| | VLLPLAFFLL LATVFSCIWK SHPSLCRKLG SILKRRPQGE GPNPVAGSWE PPKAHPYFPD | 240 |
| | LVQPLLPISG DVSPVSTGLP AAPVLEAGVP QQQSPLDLTR EPQLEPGEQS QVAHGTNGIH | 300 |
| | VTGGSMTITG NIYIYNGPVL GGPPGPGDLP ATPEPPYPIP EEGDPGPPGL STPHQEDGKA | 360 |
| | WHLAETEHCG ATPSNRGPRN QFITHD | 386 |
| SEQ ID NO: 704 | MDWTWILFLV AAATRVHSFL DSPDRPWNPP TFSPALLVVT EGDNATFTCS FSNTSESFVL | 60 |
| CCR18: ch PD-1- | NWYRMSPSNQ TDKLAAFPED RSQPGQDCRF RVTQLPNGRD FHMSVVRARR NDSGTYLCGA | 120 |
| LTBR | ISLAPKAQIK ESLRAELRVT ERRAEVPTAH PLPPEMSGTM LMLAVLLPLA FFLLLATVFS | 180 |
| | CIWKSHPSLC RKLGSLLKRR PQGEGPNPVA GSWEPPKAHP YFPDLVQPLL PISGDVSPVS | 240 |
| | TGLPAAPVLE AGVPQQQSPL DLTREPQLEP GEQSQVAHGT NGIHVTGGSM TITGNIYIYN | 300 |
| | GPVLGGPPGP GDLPATPEPP YPIPEEGDPG PPGLSTPHQE DGKAWHLAET EHCGATPSNR | 360 |
| | GPRNQFITHD | 370 |
| SEQ ID NO: 705 | MDWTWILFLV AAATRVHSTI PPHVQKSVNN DMIVTDNNGA VKFPQLCKFC DVRFSTCDNQ | 60 |
| CCR19: ch | KSCMSNCSIT SICEKPQEVC VAVWRKNDEN ITLETVCHDP KLPYHDFILE DAASPKCIMK | 120 |
| TGFBRII-LTBR | EKKKPGETFF MCSCSSDECN DNIIFSEEYN TSNPDLLLVI FQPLPPEMSG TMLMLAVLLP | 180 |
| | LAFFLLLATV FSCIWKSHPS LCRKLGSLLK RRPQGEGPNP VAGSWEPPKA HPYFPDLVQP | 240 |
| | LLPISGDVSP VSTGLPAAPV LEAGVPQQQS PLDLTREPQL EPGEQSQVAH GTNGIHVTGG | 300 |
| | SMTITGNIYI YNGPVLGGPP GPGDLPATPE PPYPIPEEGD PGPPGLSTPH QEDGKAWHLA | 360 |
| | ETEHCGATPS NRGPRNQFIT HD | 382 |

Suitable, non-limiting embodiments of nucleotides encoding the CCRs prepared according to this example and useful as CCR constructs of the present invention are set forth in Table 94.

TABLE 94
Nucleotide sequences of exemplary CCRs designated CCR17, CCR18, and
CCR19.

| Identifier | Sequence (One-Letter Nucleotide Symbols) | |
|---|---|---|
| SEQ ID NO: 706 | ATGGATTGGA CCTGGATCCT GTTCCTGGTG GCTGCGGCTA CACGGGTCCA CTCACAGGTC | 60 |
| CCR17: ch Fas- | ACCGACATTA ACTCGAAGGG CCTGGAGCTG CGCAAGACTG TGACGACCGT GGAGACCCAG | 120 |
| LTBR | AACCTGGAAG GTTTGCACCA CGATGGACAG TTTTGTCACA AGCCCTGCCC GCCTGGTGAG | 180 |
| | CGGAAGGCCC GCGACTGTAC CGTCAACGGA GACGAGCCCG ACTGCGTGCC TTGCCAGGAG | 240 |
| | GGCAAAGAGT ACACCGACAA GGCCCACTTT AGCTCCAAAT GCCGTAGGTG TCGCCTGTGC | 300 |
| | GACGAGGGCC ACGGGCTGGA GGTGGAGATC AACTGCACTC GCACCCAGAA CACGAAATGC | 360 |
| | AGATGTAAGC CTAACTTTTT CTGTAATTCC ACCGTTTGCG AGCACTGTGA TCCATGCACC | 420 |
| | AAGTGCGAGC ACGGTATCAT CAAGGAGTGC ACTCTGACCT CCAATACGAA GTGCAAGGAG | 480 |
| | GAGGGTTCCC GCAGCAATCC ACTGCCCCCC GAGATGTCTG GCACCATGCT GATGCTCGCG | 540 |
| | GTGCTGCTCC CTCTAGCGTT CTTCCTGCTG CTTGCCACCG TATTCAGCTG TATTTGGAAG | 600 |
| | AGCCACCCCT CACTGTGCCG TAAGCTCGGC TCTCTGCTGA AGCGCCGACC TCAGGGAGAG | 660 |
| | GGACCCAACC CTGTGGCTGG GAGTTGGGAG CCGCCCAAGG CCCATCCCTA TTTCCCGGAT | 720 |
| | CTTGTGCAGC CATTACTCCC CATCTCTGGT GACGTGTCCC CAGTGTCGAC CGGCCTTCCG | 780 |
| | GCCGCTCCGG TTCTAGAAGC AGGCGTGCCC CAACAGCAGT CCCCCTTGGA CCTGACTCGT | 840 |
| | GAGCCGCAGC TGGAGCCCGG GGAACAGTCC CAGGTGGCGC ATGGCACCAA CGGCATCCAT | 900 |
| | GTCACAGGCG GCTCCATGAC CATCACCGGG AACATCTACA TATACAACGG TCCGGTGCTG | 960 |
| | GGTGGGCCTC CCGGCCCTGG GGACCTTCCC GCAACTCCGG AACCACCCTA CCCAATCCCT | 1020 |
| | GAGGAGGGTG ACCCGGGACC CCCGGGCCTC TCTACCCCCC ACCAGGAGGA CGGCAAAGCC | 1080 |
| | TGGCACTTGG CCGAGACCGA GCACTGTGGC GCTACTCCTA GCAACCGCGG CCCGCGCAAC | 1140 |
| | CAGTTCATTA CTCATGAC | 1158 |
| SEQ ID NO: 707 | ATGGATTGGA CCTGGATCCT GTTTCTGGTG GCTGCTGCCA CGAGAGTGCA CTCTTTTCTC | 60 |
| CCR18: ch PD-1- | GATTCCCCCG ACCGCCCGTG GAACCCGCCC ACGTTCTCAC CGGCGCTGCT AGTAGTCACC | 120 |
| LTBR | GAGGGCGACA ACGCCACCTT CACCTGCTCG TTTTCGAACA CGTCCGAGTC CTTCGTCCTC | 180 |
| | AACTGGTACA GGATGAGCCC ATCCAACCAG ACTGACAAAC TGGCAGCGTT CCCGGAAGAC | 240 |
| | CGCTCCCAGC CTGGCCAGGA CTGCCGATTC CGCGTTACCC AGCTTCCCAA TGGCCGCGAC | 300 |
| | TTTCACATGT CCGTGGTCCG GGCTCGTCGC AACGACTCCG GTACCTACCT GTGCGGCGCC | 360 |
| | ATCTCTTTGG CACCTAAGGC CCAAATTAAA GAGAGCCTGC GCGCCGAGCT TCGGGTGACA | 420 |
| | GAGCGCCGTG CGGAGGTGCC CACTGCTCAC CCTTTGCCAC CCGAGATGTC AGGCACAATG | 480 |
| | CTGATGCTGG CTGTGCTGCT GCCACTGGCG TTCTTCCTGC TTCTAGCCAC CGTGTTCAGC | 540 |
| | TGTATTTGGA AGAGTCACCC CTCTCTGTGC CGCAAGCTGG GCTCTCTACT GAAGCGCAGG | 600 |
| | CCTCAGGGCG AGGGCCCTAA TCCAGTTGCC GGCAGTTGGG AGCCCCCCAA GGCACATCCC | 660 |
| | TATTTCCCCG ACTTGGTACA GCCGTTGCTC CCCATTTCTG GTGACGTGTC CCCGGTGTCC | 720 |
| | ACCGGTCTCC CCGCTGCCCC AGTCCTGGAG GCTGGGGTGC CTCAGCAGCA GAGCCCCCTG | 780 |
| | GACCTGACCC GTGAACCTCA GCTGGAGCCA GGAGAACAGA GCCAGGTGGC GCATGGAACC | 840 |
| | AACGGCATCC ACGTCACTGG AGGGTCCATG ACCATCACCG GCAACATCTA CATCTACAAC | 900 |
| | GGGCCAGTGC TGGGAGGTCC CCCAGGCCCT GGCGATCTGC CGGCTACCCC GGAGCCTCCT | 960 |
| | TACCCTATCC CCGAGGAGGG CGACCCGGGG CCGCCCGGCT TATCGACCCC TCACCAGGAG | 1020 |
| | GACGGCAAGG CCTGGCACCT CGCCGAGACT GAGCACTGTG GGGCCACCCC CAGCAACCGC | 1080 |
| | GGCCCGCGCA ATCAGTTCAT CACTCATGAC | 1110 |
| SEQ ID NO: 708 | ATGGATTGGA CCTGGATCCT ATTTCTGGTG GCGGCTGCGA CTCGGGTGCA CTCGACCATC | 60 |
| CCR19: ch | CCGCCGCACG TTCAGAAGTC CGTTAACAAC GACATGATCG TGACCGACAA CAATGGCGCC | 120 |
| TGFBRII-LTBR | GTGAAATTCC CCCAGCTGTG CAAGTTTTGC GACGTGCGCT TTTCAACCTG CGACAACCAG | 180 |
| | AAGTCATGCA TGAGCAACTG CTCTATTACC AGCATCTGCG AGAAGCCTCA GGAGGTGTGC | 240 |
| | GTGGCTGTCT GGCGTAAGAA CGACGAGAAC ATCACCCTGG AGACCGTCTG CCACGACCCG | 300 |
| | AAGTTACCCT ATCACGATTT CATACTGGAA GACGCAGCCT CCCCGAAGTG CATCATGAAG | 360 |
| | GAGAAAAAGA AGCCTGGGGA GACCTTCTTC ATGTGCTCCT GTTCCTCGGA CGAGTGTAAC | 420 |
| | GACAACATCA TCTTTAGCGA AGAGTACAAC ACGTCCAATC CAGACCTGCT CCTGGTAATC | 480 |
| | TTCCAGCCTC TGCCCCCCGA GATGTCTGGC ACCATGCTGA TGTTGGCCGT CCTCCTTCCG | 540 |
| | CTTGCGTTCT TCCTGCTTCT GGCCACCGTG TTCAGTTGTA TTTGGAAGAG CCACCCTTCT | 600 |
| | CTGTGCCGAA AGCTGGGCTC CTTGCTCAAA CGCAGGCCCC AGGGCGAGGG ACCCAATCCA | 660 |
| | GTGGCTGGCA GTTGGGAGCC GCCCAAGGCC CATCCCTACT TCCCCGACCT GGTGCAGCCT | 720 |
| | TTGTTACCAA TCTCTGGTGA TGTCAGCCCC GTGTCCACAG GCCTGCCTGC CGCGCCGGTG | 780 |
| | CTGGAGGCCG GGGTGCCTCA GCAGCAGTCG CCCCTAGACC TGACCCGCGA GCCCCAACTG | 840 |
| | GAGCCAGGAG AACAGAGCCA GGTCGCGCAT GGAACTAATG GCATCCACGT CACCGGTGGC | 900 |
| | TCCATGACGA TCACCGGCAA CATTTACATC TACAACGGTC CGGTGCTGGG CGGCCCTCCG | 960 |
| | GGGCCTGGTG ATCTGCCGGC CACGCCTGAG CCCCCCTACC CCATCCCGGA GGAGGGAGAT | 020 |
| | CCAGGGCCAC CTGGCCTCTC CACCCCCCAC CAGGAGGACG GCAAAGCTTG GCACCTCGCA | 1080 |
| | GAGACTGAGC ACTGTGGCGC TACTCCCTCT AACCGCGGTC CCCGCAACCA GTTCATTACT | 1140 |
| | CATGAC | 1146 |

Vectors encoding the CCRs designated CCR17, CCR18, and CCR19 were prepared as described above. The full nucleotide sequences of these vectors are presented in Table 95.

TABLE 95
Nucleotide sequences of exemplary vectors encoding CCRs designated CCR17,
CCR18, and CCR19.

| Identifier | Sequence (One-Letter Nucleotide Symbols) | |
|---|---|---|
| SEQ ID NO: 709 | GTCGACGGAT CGGGAGATCT CCCGATCCCC TATGGTGCAC TCTCAGTACA ATCTGCTCTG | 60 |
| CCR17: ch FAS- | ATGCCGCATA GTTAAGCCAG TATCTGCTCC CTGCTTGTGT GTTGGAGGTC GCTGAGTAGT | 120 |
| LTBR | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGASTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCATGGATT GGACCTGGAT CCTGTTTCTG GTGGCTGCTG CCACGAGAGT | 2940 |
| | GCACTCTTTT CTCGATTCCC CCGACCGCCC GTGGAACCCG CCCACGTTCT CACCGGCGCT | 3000 |
| | GCTAGTAGTC ACCGAGGGCG ACAACGCCAC CTTCACCTGC TCGTTTTCGA ACACGTCCGA | 3060 |
| | GTCCTTCGTC CTCAACTGGT ACAGGATGAG CCCATCCAAC CAGACTGACA AACTGGCAGC | 3120 |
| | GTTCCCGGAA GACCGCTCCC AGCCTGGCCA GGACTGCCGA TTCCGCGTTA CCCAGCTTCC | 3180 |
| | CAATGGCCGC GACTTTCACA TGTCCGTGGT CCGGGCTCGT CGCAACGACT CCGGTACCTA | 3240 |
| | CCTGTGCGGC GCCATCTCTT TGGCACCTAA GGCCCAAATT AAAGAGAGCC TGCGCGCCGA | 3300 |
| | GCTTCGGGTG ACAGAGCGCC GTGCGGAGGT GCCCACTGCT CACCCTTTGC CACCCGAGAT | 3360 |
| | GTCAGGCACA ATGCTGATGC TGGCTGTGCT GCTGCCACTG GCGTTCTTCC TGCTTCTAGC | 3420 |
| | CACCGTGTTC AGCTGTATTT GGAAGAGTCA CCCCTCTCTG TGCCGCAAGC TGGGCTCTCT | 3480 |
| | ACTGAAGCGC AGGCCTCAGG GCGAGGGCCC TAATCCAGTT GCCGGCAGTT GGGAGCCCCC | 3540 |
| | CAAGGCACAT CCCTATTTCC CCGACTTGGT ACAGCCGTTG CTCCCCATTT CTGGTGACGT | 3600 |
| | GTCCCCGGTG TCCACCGGTC TCCCCGCTGC CCCAGTCCTG GAGGCTGGGG TGCCTCAGCA | 3660 |
| | GCAGAGCCCC CTGGACCTGA CCCGTGAACC TCAGCTGGAG CCAGGAGAAC AGAGCCAGGT | 3720 |
| | GGCGCATGGA ACCAACGGCA TCCACGTCAC TGGAGGGTCC ATGACCATCA CCGGCAACAT | 3780 |
| | CTACATCTAC AACGGGCCAG TGCTGGGAGG TCCCCCAGGC CCTGGCGATC TGCCGGCTAC | 3840 |
| | CCCGGAGCCT CCTTACCCTA TCCCCGAGGA GGGCGACCCG GGGCCGCCCG GCTTATCGAC | 3900 |
| | CCCTCACCAG GAGGACGGCA AGGCCTGGCA CCTCGCCGAG ACTGAGCACT GTGGGGCCAC | 3960 |
| | CCCCAGCAAC CGCGGCCCGC GCAATCAGTT CATCACTCAT GACTGAGAAT TCCGCCCCCC | 4020 |
| | CCCCCCCCCC CCCTCTCCCT CCCCCCCCCC TAACGTTACT GGCCGAAGCC GCTTGGAATA | 4080 |
| | AGGCCGGTGT GCGTTTGTCT ATATGTTATT TTCCACCATA TTGCCGTCTT TTGGCAATGT | 4140 |
| | GAGGGCCCGG AAACCTGGCC CTGTCTTCTT GACGAGCATT CCTAGGGGTC TTTCCCCTCT | 4200 |
| | CGCCAAAGGA ATGCAAGGTC TGTTGAATGT CGTGAAGGAA GCAGTTCCTC TGGAAGCTTC | 4260 |
| | TTGAAGACAA ACAACGTCTG TAGCGACCCT TTGCAGGCAG CGGAACCCCC CACCTGGCGA | 4320 |
| | CAGGTGCCTC TGCGGCCAAA AGCCACGTGT ATAAGATACA CCTGCAAAGG CGGCACAACC | 4380 |
| | CCAGTGCCAC GTTGTGAGTT GGATAGTTGT GGAAAGAGTC AAATGGCTCT CCTCAAGCGT | 4440 |
| | ATTCAACAAG GGGCTGAAGG ATGCCCAGAA GGTACCCCAT TGTATGGGAT CTGATCTGGG | 4500 |
| | GCCTCGGTGC ACATGCTTTA CATGTGTTTA GTCGAGGTTA AAAAAACGTC TAGGCCCCCC | 4560 |
| | GAACCACGGG GACGTGGTTT TCCTTTGAAA AACACGATGA TAAGCTTGAT CACGCGTGCC | 4620 |
| | ACCATGAGCA AGGGCGAGGA GCTGTTCACC GGGGTGGTGC CCATCCTGGT CGAGCTGGAC | 4680 |
| | GGCGACGTAA ACGGCCACAA GTTCAGCGTG TCCGGCGAGG GCGAGGGCGA TGCCACCTAC | 4740 |
| | GGCAAGCTGA CCCTGAAGTT CATCTGCACC ACCGGCAAGC TGCCCGTGCC CTGGCCCACC | 4800 |
| | CTCGTGACCA CCCTGACCTA CGGCGTGCAG TGCTTCAGCC GCTACCCCGA CCACATGAAG | 4860 |
| | CAGCACGACT TCTTCAAGTC CGCCATGCCC GAAGGCTACG TCCAGGAGCG CACCATCTTC | 4920 |
| | TTCAAGGACG ACGGCAACTA CAAGACCCGC GCCGAGGTGA AGTTCGAGGG CGACACCCTG | 4980 |
| | GTGAACCGCA TCGAGCTGAA GGGCATCGAC TTCAAGGAGG ACGGCAACAT CCTGGGGCAC | 5040 |
| | AAGCTGGAGT ACAACTACAA CAGCCACAAC GTCTATATCA TGGCCGACAA GCAGAAGAAC | 5100 |
| | GGCATCAAGG TGAACTTCAA GATCCGCCAC AACATCGAGG ACGGCAGCGT GCAGCTCGCC | 5160 |
| | GACCACTACC AGCAGAACAC CCCCATCGGC GACGGCCCCG TGCTGCTGCC CGACAACCAC | 5220 |
| | TACCTGAGCA CCCAGTCCGC CCTGAGCAAA GACCCCAACG AGAAGCGCGA TCACATGGTC | 5280 |
| | CTGCTGGAGT TCGTGACCGC CGCCGGGATC ACTCACGGCA TGGACGAGCT GTACAAGTGA | 5340 |
| | GAATTCGATA TCAAGCTTAT CGGTAATCAA CCTCTGGATT ACAAAATTTG TGAAAGATTG | 5400 |
| | ACTGGTATTC TTAACTATGT TGCTCCTTTT ACGCTATGTG GATACGCTGC TTTAATGCCT | 5460 |
| | TTGTATCATG CTATTGCTTC CCGTATGGCT TTCATTTTCT CCTCCTTGTA TAAATCCTGG | 5520 |
| | TTGCTGTCTC TTTATGAGGA GTTGTGGCCC GTTGTCAGGC AACGTGGCGT GGTGTGCACT | 5580 |
| | GTGTTTGCTG ACGCAACCCC CACTGGTTGG GGCATTGCCA CCACCTGTCA GCTCCTTTCC | 5640 |
| | GGGACTTTCG CTTTCCCCCT CCCTATTGCC ACGGCGGAAC TCATCGCCGC CTGCCTTGCC | 5700 |
| | CGCTGCTGGA CAGGGGCTCG GCTGTTGGGC ACTGACAATT CCGTGGTGTT GTCGGGGAAA | 5760 |
| | TCATCGTCCT TTCCTTGGCT GCTCGCCTGT GTTGCCACCT GGATTCTGCG CGGGACGTCC | 5820 |
| | TTCTGCTACG TCCCTTCGGC CCTCAATCCA GCGGACCTTC CTTCCCGCGG CCTGCTGCCG | 5880 |
| | GCTCTGCGGC CTCTTCCGCG TCTTCGCCTT CGCCCTCAGA CGAGTCGGAT CTCCCTTTGG | 5940 |
| | GCCGCCTCCC CGCATCGATA CCGTCGACCT CGAGACCTAG AAAAACATGG AGCAATCACA | 6000 |
| | AGTAGCAATA CAGCAGCTAC CAATGCTGAT TGTGCCTGGC TAGAAGCACA AGAGGAGGAG | 6060 |
| | GAGGTGGGTT TTCCAGTCAC ACCTCAGGTA CCTTTAAGAC CAATGACTTA CAAGGCAGCT | 6120 |
| | GTAGATCTTA GCCACTTTTT AAAAGAAAAG GGGGGACTGG AAGGGCTAAT TCACTCCCAA | 6180 |
| | CGAAGACAAG ATATCCTTGA TCTGTGGATC TACCACACAC AAGGCTACTT CCCTGATTGG | 6240 |
| | CAGAACTACA CACCAGGGCC AGGGATCAGA TATCCACTGA CCTTTGGATG GTGCTACAAG | 6300 |
| | CTAGTACCAG TTGAGCAAGA GAAGGTAGAA GAAGCCAATG AAGGAGAGAA CACCCGCTTG | 6360 |
| | TTACACCCTG TGAGCCTGCA TGGGATGGAT GACCCGGAGA GAGAAGTATT AGAGTGGAGG | 6420 |
| | TTTGACAGCC GCCTAGCATT TCATCACATG GCCCGAGAGC TGCATCCGGA CTGTACTGGG | 6480 |
| | TCTCTCTGGT TAGACCAGAT CTGAGCCTGG GAGCTCTCTG GCTAACTAGG GAACCCACTG | 6540 |
| | CTTAAGCCTC AATAAAGCTT GCCTTGAGTG CTTCAAGTAG TGTGTGCCCG TCTGTTGTGT | 6600 |
| | GACTCTGGTA ACTAGAGATC CCTCAGACCC TTTTAGTCAG TGTGGAAAAT CTCTAGCAGG | 6660 |
| | GCCCGTTTAA ACCCGCTGAT CAGCCTCGAC TGTGCCTTCT AGTTGCCAGC CATCTGTTGT | 6720 |
| | TTGCCCCTCC CCCGTGCCTT CCTTGACCCT GGAAGGTGCC ACTCCCACTG TCCTTTCCTA | 6780 |
| | ATAAAATGAG GAAATTGCAT CGCATTGTCT GAGTAGGTGT CATTCTATTC TGGGGGGTGG | 6840 |
| | GGTGGGGCAG GACAGCAAGG GGGAGGATTG GGAAGACAAT AGCAGGCATG CTGGGGATGC | 6900 |
| | GGTGGGCTCT ATGGCTTCTG AGGCGGAAAG AACCAGCTGG GGCTCTAGGG GGTATCCCCA | 6960 |
| | CGCGCCCTGT AGCGGCGCAT TAAGCGCGGC GGGTGTGGTG GTTACGCGCA GCGTGACCGC | 7020 |
| | TACACTTGCC AGCGCCCTAG CGCCCGCTCC TTTCGCTTTC TTCCCTTCCT TTCTCGCCAC | 7080 |
| | GTTCGCCGGC TTTCCCCGTC AAGCTCTAAA TCGGGGGCTC CCTTTAGGGT TCCGATTTAG | 7140 |
| | TGCTTTACGG CACCTCGACC CCAAAAAACT TGATTAGGGT GATGGTTCAC GTAGTGGGCC | 7200 |
| | ATCGCCCTGA TAGACGGTTT TTCGCCCTTT GACGTTGGAG TCCACGTTCT TTAATAGTGG | 7260 |
| | ACTCTTGTTC CAAACTGGAA CAACACTCAA CCCTATCTCG GTCTATTCTT TTGATTTATA | 7320 |
| | AGGGATTTTG CCGATTTCGG CCTATTGGTT AAAAAATGAG CTGATTTAAC AAAAATTTAA | 7380 |
| | CGCGAATTAA TTCTGTGGAA TGTGTGTCAG TTAGGGTGTG GAAAGTCCCC AGGCTCCCCA | 7440 |
| | GCAGGCAGAA GTATGCAAAG CATGCATCTC AATTAGTCAG CAACCAGGTG TGGAAAGTCC | 7500 |
| | CCAGGCTCCC CAGCAGGCAG AAGTATGCAA AGCATGCATC TCAATTAGTC AGCAACCATA | 7560 |
| | GTCCCGCCCC TAACTCCGCC CATCCCGCCC CTAACTCCGC CCAGTTCCGC CCATTCTCCG | 7620 |
| | CCCCATGGCT GACTAATTTT TTTTATTTAT GCAGAGGCCG AGGCCGCCTC TGCCTCTGAG | 7680 |
| | CTATTCCAGA AGTAGTGAGG AGGCTTTTTT GGAGGCCTAG GCTTTTGCAA AAAGCTCCCG | 7740 |
| | GGAGCTTGTA TATCCATTTT CGGATCTGAT CAGCACGTGT TGACAATTAA TCATCGGCAT | 7800 |
| | AGTATATCGG CATAGTATAA TACGACAAGG TGAGGAACTA AACCATGGCC AAGTTGACCA | 7860 |
| | GTGCCGTTCC GGTGCTCACC GCGCGCGACG TCGCCGGAGC GGTCGAGTTC TGGACCGACC | 7920 |
| | GGCTCGGGTT CTCCCGGGAC TTCGTGGAGG ACGACTTCGC CGGTGTGGTC CGGGACGACG | 7980 |
| | TGACCCTGTT CATCAGCGCG GTCCAGGACC AGGTGGTGCC GGACAACACC CTGGCCTGGG | 8040 |
| | TGTGGGTGCG CGGCCTGGAC GAGCTGTACG CCGAGTGGTC GGAGGTCGTG TCCACGAACT | 8100 |
| | TCCGGGACGC CTCCGGGCCG GCCATGACCG AGATCGGCGA GCAGCCGTGG GGGCGGGAGT | 8160 |
| | TCGCCCTGCG CGACCCGGCC GGCAACTGCG TGCACTTCGT GGCCGAGGAG CAGGACTGAC | 8220 |
| | ACGTGCTACG AGATTTCGAT TCCACCGCCG CCTTCTATGA AAGGTTGGGC TTCGGAATCG | 8280 |
| | TTTTCCGGGA CGCCGGCTGG ATGATCCTCC AGCGCGGGGA TCTCATGCTG GAGTTCTTCG | 8340 |
| | CCCACCCCAA CTTGETTATT GCAGCTTATA ATGGTTACAA ATAAAGCAAT AGCATCACAA | 8400 |
| | ATTTCACAAA TAAAGCATTT TTTTCACTGC ATTCTAGTTG TGGTTTGTCC AAACTCATCA | 8460 |
| | ATGTATCTTA TCATGTCTGT ATACCGTCGA CCTCTAGCTA GAGCTTGGCG TAATCATGGT | 8520 |
| | CATAGCTGTT TCCTGTGTGA AATTGTTATC CGCTCACAAT TCCACACAAC ATACGAGCCG | 8580 |
| | GAAGCATAAA GTGTAAAGCC TGGGGTGCCT AATGAGTGAG CTAACTCACA TTAATTGCGT | 8640 |
| | TGCGCTCACT GCCCGCTTTC CAGTCGGGAA ACCTGTCGTG CCAGCTGCAT TAATGAATCG | 8700 |
| | GCCAACGCGC GGGGAGAGGC GGTTTGCGTA TTGGGCGCTC TTCCGCTTCC TCGCTCACTG | 8760 |
| | ACTCGCTGCG CTCGGTCGTT CGGCTGCGGC GAGCGGTATC AGCTCACTCA AAGGCGGTAA | 8820 |
| | TACGGTTATC CACAGAATCA GGGGATAACG CAGGAAAGAA CATGTGAGCA AAAGGCCAGC | 8880 |
| | AAAAGGCCAG GAACCGTAAA AAGGCCGCGT TGCTGGCGTT TTTCCATAGG CTCCGCCCCC | 8940 |
| | CTGACGAGCA TCACAAAAAT CGACGCTCAA GTCAGAGGTG GCGAAACCCG ACAGGACTAT | 9000 |
| | AAAGATACCA GGCGETTCCC CCTGGAAGCT CCCTCGTGCG CTCTCCTGTT CCGACCCTGC | 9060 |
| | CGCTTACCGG ATACCTGTCC GCCTTTCTCC CTTCGGGAAG CGTGGCGCTT TCTCATAGCT | 9120 |
| | CACGCTGTAG GTATCTCAGT TCGGTGTAGG TCGTTCGCTC CAAGCTGGGC TGTGTGCACG | 9180 |
| | AACCCCCCGT TCAGCCCGAC CGCTGCGCCT TATCCGGTAA CTATCGTCTT GAGTCCAACC | 9240 |
| | CGGTAAGACA CGACTTATCG CCACTGGCAG CAGCCACTGG TAACAGGATT AGCAGAGCGA | 9300 |
| | GGTATGTAGG CGGTGCTACA GAGTTCTTGA AGTGGTGGCC TAACTACGGC TACACTAGAA | 9360 |
| | GAACAGTATT TGGTATCTGC GCTCTGCTGA AGCCAGTTAC CTTCGGAAAA AGAGTTGGTA | 9420 |
| | GCTCTTGATC CGGCAAACAA ACCACCGCTG GTAGCGGTGG TTTTTTTGTT TGCAAGCAGC | 9480 |
| | AGATTACGCG CAGAAAAAAA GGATCTCAAG AAGATCCTTT GATCTTTTCT ACGGGGTCTG | 9540 |
| | ACGCTCAGTG GAACGAAAAC TCACGTTAAG GGATTTTGGT CATGAGATTA TCAAAAAGGA | 9600 |
| | TCTTCACCTA GATCCTTTTA AATTAAAAAT GAAGTTTTAA ATCAATCTAA AGTATATATG | 9660 |
| | AGTAAACTTG GTCTGACAGT TACCAATGCT TAATCAGTGA GGCACCTATC TCAGCGATCT | 9720 |
| | GTCTATTTCG TTCATCCATA GTTGCCTGAC TCCCCGTCGT GTAGATAACT ACGATACGGG | 9780 |
| | AGGGCTTACC ATCTGGCCCC AGTGCTGCAA TGATACCGCG AGACCCACGC TCACCGGCTC | 9840 |
| | CAGATTTATC AGCAATAAAC CAGCCAGCCG GAAGGGCCGA GCGCAGAAGT GGTCCTGCAA | 9900 |
| | CTTTATCCGC CTCCATCCAG TCTATTAATT GTTGCCGGGA AGCTAGAGTA AGTAGTTCGC | 9960 |
| | CAGTTAATAG TTTGCGCAAC GTTGTTGCCA TTGCTACAGG CATCGTGGTG TCACGCTCGT | 10020 |
| | CGTTTGGTAT GGCTTCATTC AGCTCCGGTT CCCAACGATC AAGGCGAGTT ACATGATCCC | 10080 |
| | CCATGTTGTG CAAAAAAGCG GTTAGCTCCT TCGGTCCTCC GATCGTTGTC AGAAGTAAGT | 10140 |
| | TGGCCGCAGT GTTATCACTC ATGGTTATGG CAGCACTGCA TAATTCTCTT ACTGTCATGC | 10200 |
| | CATCCGTAAG ATGCTTTTCT GTGACTGGTG AGTACTCAAC CAAGTCATTC TGAGAATAGT | 10260 |
| | GTATGCGGCG ACCGAGTTGC TCTTGCCCGG CGTCAATACG GGATAATACC GCGCCACATA | 10320 |
| | GCAGAACTTT AAAAGTGCTC ATCATTGGAA AACGTTCTTC GGGGCGAAAA CTCTCAAGGA | 10380 |
| | TCTTACCGCT GTTGAGATCC AGTTCGATGT AACCCACTCG TGCACCCAAC TGATCTTCAG | 10440 |
| | CATCTTTTAC TTTCACCAGC GTTTCTGGGT GAGCAAAAAC AGGAAGGCAA AATGCCGCAA | 10500 |
| | AAAAGGGAAT AAGGGCGACA CGGAAATGTT GAATACTCAT ACTCTTCCTT TTTCAATATT | 10560 |
| | ATTGAAGCAT TTATCAGGGT TATTGTCTCA TGAGCGGATA CATATTTGAA TGTATTTAGA | 10620 |
| | AAAATAAACA AATAGGGGTT CCGCGCACAT TTCCCCGAAA AGTGCCACCT GAC | 10673 |
| SEQ ID NO: 710 | GTCGACGGAT CGGGAGATCT CCCGATCCCC TATGGTGCAC TCTCAGTACA ATCTGCTCTG | 60 |
| CCR18: ch PD-1- | ATGCCGCATA GTTAAGCCAG TATCTGCTCC CTGCTTGTGT GTTGGAGGTC GCTGAGTAGT | 120 |
| LTBR | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGASTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCAEGGATT GGACCTGGAT CCTATTTCTG GTGGCGGCTG CGACTCGGGT | 2940 |
| | GCACTCGACC ATCCCGCCGC ACGTTCAGAA GTCCGTTAAC AACGACATGA TCGTGACCGA | 3000 |
| | CAACAATGGC GCCGTGAAAT TCCCCCAGCT GTGCAAGTTT TGCGACGTGC GCTTTTCAAC | 3060 |
| | CTGCGACAAC CAGAAGTCAT GCATGAGCAA CTGCTCTATT ACCAGCATCT GCGAGAAGCC | 3120 |
| | TCAGGAGGTG TGCGTGGCTG TCTGGCGTAA GAACGACGAG AACATCACCC TGGAGACCGT | 3180 |
| | CTGCCACGAC CCGAAGTTAC CCTATCACGA TTTCATACTG GAAGACGCAG CCTCCCCGAA | 3240 |
| | GTGCATCATG AAGGAGAAAA AGAAGCCTGG GGAGACCTTC TTCATGTGCT CCTGTTCCTC | 3300 |
| | GGACGAGTGT AACGACAACA TCATCTTTAG CGAAGAGTAC AACACGTCCA ATCCAGACCT | 3360 |
| | GCTCCTGGTA ATCTTCCAGC CTCTGCCCCC CGAGATGTCT GGCACCATGC TGATGTTGGC | 3420 |
| | CGTCCTCCTT CCGCTTGCGT TCTTCCTGCT TCTGGCCACC GTGTTCAGTT GTATTTGGAA | 3480 |
| | GAGCCACCCT TCTCTGTGCC GAAAGCTGGG CTCCTTGCTC AAACGCAGGC CCCAGGGCGA | 3540 |
| | GGGACCCAAT CCAGTGGCTG GCAGTTGGGA GCCGCCCAAG GCCCATCCCT ACTTCCCCGA | 3600 |
| | CCTGGTGCAG CCTTIGTTAC CAATCTCTGG TGATGTCAGC CCCGTGTCCA CAGGCCTGCC | 3660 |
| | TGCCGCGCCG GTGCTGGAGG CCGGGGTGCC TCAGCAGCAG TCGCCCCTAG ACCTGACCCG | 3720 |
| | CGAGCCCCAA CTGGAGCCAG GAGAACAGAG CCAGGTCGCG CATGGAACTA ATGGCATCCA | 3780 |
| | CGTCACCGGT GGCTCCATGA CGATCACCGG CAACATTTAC ATCTACAACG GTCCGGTGCT | 3840 |
| | GGGCGGCCCT CCGGGGCCTG GTGATCTGCC GGCCACGCCT GAGCCCCCCT ACCCCATCCC | 3900 |
| | GGAGGAGGGA GATCCAGGGC CACCTGGCCT CTCCACCCCC CACCAGGAGG ACGGCAAAGC | 3960 |
| | TTGGCACCTC GCAGAGACTG AGCACTGTGG CGCTACTCCC TCTAACCGCG GTCCCCGCAA | 4020 |
| | CCAGTTCATT ACTCATGACT GAGAATTCCG CCCCCCCCCC CCCCCCCCCT CTCCCTCCCC | 4080 |
| | CCCCCCTAAC GTTACTGGCC GAAGCCGCTT GGAATAAGGC CGGTGTGCGT TTGTCTATAT | 4140 |
| | GTTATTTTCC ACCATATTGC CGTCTTTTGG CAATGTGAGG GCCCGGAAAC CTGGCCCTGT | 4200 |
| | CTTCTTGACG AGCATTCCTA GGGGTCTTTC CCCTCTCGCC AAAGGAATGC AAGGTCTGTT | 4260 |
| | GAATGTCGTG AAGGAAGCAG TTCCTCTGGA AGCTTCTTGA AGACAAACAA CGTCTGTAGC | 4320 |
| | GACCCTTTGC AGGCAGCGGA ACCCCCCACC TGGCGACAGG TGCCTCTGCG GCCAAAAGCC | 4380 |
| | ACGTGTATAA GATACACCTG CAAAGGCGGC ACAACCCCAG TGCCACGTTG TGAGTTGGAT | 4440 |
| | AGTTGTGGAA AGAGTCAAAT GGCTCTCCTC AAGCGTATTC AACAAGGGGC TGAAGGATGC | 4500 |
| | CCAGAAGGTA CCCCATTGTA TGGGATCTGA TCTGGGGCCT CGGTGCACAT GCTTTACATG | 4560 |
| | TGTTTAGTCG AGGTTAAAAA AACGTCTAGG CCCCCCGAAC CACGGGGACG TGGTTTTCCT | 4620 |
| | TTGAAAAACA CGATGATAAG CTTGATCACG CGTGCCACCA TGAGCAAGGG CGAGGAGCTG | 4680 |
| | TTCACCGGGG TGGTGCCCAT CCTGGTCGAG CTGGACGGCG ACGTAAACGG CCACAAGTTC | 4740 |
| | AGCGTGTCCG GCGAGGGCGA GGGCGATGCC ACCTACGGCA AGCTGACCCT GAAGTTCATC | 4800 |
| | TGCACCACCG GCAAGCTGCC CGTGCCCTGG CCCACCCTCG TGACCACCCT GACCTACGGC | 4860 |
| | GTGCAGTGCT TCAGCCGCTA CCCCGACCAC ATGAAGCAGC ACGACTTCTT CAAGTCCGCC | 4920 |
| | ATGCCCGAAG GCTACGTCCA GGAGCGCACC ATCTTCTTCA AGGACGACGG CAACTACAAG | 4980 |
| | ACCCGCGCCG AGGTGAAGTT CGAGGGCGAC ACCCTGGTGA ACCGCATCGA GCTGAAGGGC | 5040 |
| | ATCGACTTCA AGGAGGACGG CAACATCCTG GGGCACAAGC TGGAGTACAA CTACAACAGC | 5100 |
| | CACAACGTCT ATATCATGGC CGACAAGCAG AAGAACGGCA TCAAGGTGAA CTTCAAGATC | 5160 |
| | CGCCACAACA TCGAGGACGG CAGCGTGCAG CTCGCCGACC ACTACCAGCA GAACACCCCC | 5220 |
| | ATCGGCGACG GCCCCGTGCT GCTGCCCGAC AACCACTACC TGAGCACCCA GTCCGCCCTG | 5280 |
| | AGCAAAGACC CCAACGAGAA GCGCGATCAC ATGGTCCTGC TGGAGTTCGT GACCGCCGCC | 5340 |
| | GGGATCACTC ACGGCATGGA CGAGCTGTAC AAGTGAGAAT TCGATATCAA GCTTATCGGT | 5400 |
| | AATCAACCTC TGGATTACAA AATTTGTGAA AGATTGACTG GTATTCTTAA CTATGTTGCT | 5460 |
| | CCTTTTACGC TATGEGGATA CGCTGCTTTA ATGCCTTTGT ATCATGCTAT TGCTTCCCGT | 5520 |
| | ATGGCTTTCA TTTTCTCCTC CTTGTATAAA TCCTGGTTGC TGTCTCTTTA TGAGGAGTTG | 5580 |
| | TGGCCCGTTG TCAGGCAACG TGGCGTGGTG TGCACTGTGT TTGCTGACGC AACCCCCACT | 5640 |
| | GGTTGGGGCA TTGCCACCAC CTGTCAGCTC CTTTCCGGGA CTTTCGCTTT CCCCCTCCCT | 5700 |
| | ATTGCCACGG CGGAACTCAT CGCCGCCTGC CTTGCCCGCT GCTGGACAGG GGCTCGGCTG | 5760 |
| | TTGGGCACTG ACAATTCCGT GGTGTTGTCG GGGAAATCAT CGTCCTTTCC TTGGCTGCTC | 5820 |
| | GCCTGTGTTG CCACCTGGAT TCTGCGCGGG ACGTCCTTCT GCTACGTCCC TTCGGCCCTC | 5880 |
| | AATCCAGCGG ACCTTCCTTC CCGCGGCCTG CTGCCGGCTC TGCGGCCTCT TCCGCGTCTT | 5940 |
| | CGCCTTCGCC CTCAGACGAG TCGGATCTCC CTTTGGGCCG CCTCCCCGCA TCGATACCGT | 6000 |
| | CGACCTCGAG ACCTAGAAAA ACATGGAGCA ATCACAAGTA GCAATACAGC AGCTACCAAT | 6060 |
| | GCTGATTGTG CCTGGCTAGA AGCACAAGAG GAGGAGGAGG TGGGTTTTCC AGTCACACCT | 6120 |
| | CAGGTACCTT TAAGACCAAT GACTTACAAG GCAGCTGTAG ATCTTAGCCA CTTTTTAAAA | 6180 |
| | GAAAAGGGGG GACTGGAAGG GCTAATTCAC TCCCAACGAA GACAAGATAT CCTTGATCTG | 6240 |
| | TGGATCTACC ACACACAAGG CTACTTCCCT GATTGGCAGA ACTACACACC AGGGCCAGGG | 6300 |
| | ATCAGATATC CACTGACCTT TGGATGGTGC TACAAGCTAG TACCAGTTGA GCAAGAGAAG | 6360 |
| | GTAGAAGAAG CCAATGAAGG AGAGAACACC CGCTTGTTAC ACCCTGTGAG CCTGCATGGG | 6420 |
| | ATGGATGACC CGGAGAGAGA AGTATTAGAG TGGAGGTTTG ACAGCCGCCT AGCATTTCAT | 6480 |
| | CACATGGCCC GAGAGCTGCA TCCGGACTGT ACTGGGTCTC TCTGGTTAGA CCAGATCTGA | 6540 |
| | GCCTGGGAGC TCTCTGGCTA ACTAGGGAAC CCACTGCTTA AGCCTCAATA AAGCTTGCCT | 6600 |
| | TGAGTGCTTC AAGTAGTGTG TGCCCGTCTG TTGTGTGACT CTGGTAACTA GAGATCCCTC | 6660 |
| | AGACCCTTTT AGTCAGTGTG GAAAATCTCT AGCAGGGCCC GTTTAAACCC GCTGATCAGC | 6720 |
| | CTCGACTGTG CCTTCTAGTT GCCAGCCATC TGTTGTTTGC CCCTCCCCCG TGCCTTCCTT | 6780 |
| | GACCCTGGAA GGTGCCACTC CCACTGTCCT TTCCTAATAA AATGAGGAAA TTGCATCGCA | 6840 |
| | TTGTCTGAGT AGGTGTCATT CTATTCTGGG GGGTGGGGTG GGGCAGGACA GCAAGGGGGA | 6900 |
| | GGATTGGGAA GACAATAGCA GGCATGCTGG GGATGCGGTG GGCTCTATGG CTTCTGAGGC | 6960 |
| | GGAAAGAACC AGCTGGGGCT CTAGGGGGTA TCCCCACGCG CCCTGTAGCG GCGCATTAAG | 7020 |
| | CGCGGCGGGT GTGGTGGTTA CGCGCAGCGT GACCGCTACA CTTGCCAGCG CCCTAGCGCC | 7080 |
| | CGCTCCTTTC GCTTTCTTCC CTTCCTTTCT CGCCACGTTC GCCGGCTTTC CCCGTCAAGC | 7140 |
| | TCTAAATCGG GGGCTCCCTT TAGGGTTCCG ATTTAGTGCT TTACGGCACC TCGACCCCAA | 7200 |
| | AAAACTTGAT TAGGGTGATG GTTCACGTAG TGGGCCATCG CCCTGATAGA CGGTTTTTCG | 7260 |
| | CCCTTTGACG TTGGAGTCCA CGTTCTTTAA TAGTGGACTC TTGTTCCAAA CTGGAACAAC | 7320 |
| | ACTCAACCCT ATCTCGGTCT ATTCTTTTGA TTTATAAGGG ATTTTGCCGA TTTCGGCCTA | 7380 |
| | TTGGTTAAAA AATGAGCTGA TTTAACAAAA ATTTAACGCG AATTAATTCT GTGGAATGTG | 7440 |
| | TGTCAGTTAG GGTGTGGAAA GTCCCCAGGC TCCCCAGCAG GCAGAAGTAT GCAAAGCATG | 7500 |
| | CATCTCAATT AGTCAGCAAC CAGGTGTGGA AAGTCCCCAG GCTCCCCAGC AGGCAGAAGT | 7560 |
| | ATGCAAAGCA TGCATCTCAA TTAGTCAGCA ACCATAGTCC CGCCCCTAAC TCCGCCCATC | 7620 |
| | CCGCCCCTAA CTCCGCCCAG TTCCGCCCAT TCTCCGCCCC ATGGCTGACT AATTTTTTTT | 7680 |
| | ATTTATGCAG AGGCCGAGGC CGCCTCTGCC TCTGAGCTAT TCCAGAAGTA GTGAGGAGGC | 7740 |
| | TTTTTTGGAG GCCTAGGCTT TTGCAAAAAG CTCCCGGGAG CTTGTATATC CATTTTCGGA | 7800 |
| | TCTGATCAGC ACGTGTTGAC AATTAATCAT CGGCATAGTA TATCGGCATA GTATAATACG | 7860 |
| | ACAAGGTGAG GAACTAAACC ATGGCCAAGT TGACCAGTGC CGTTCCGGTG CTCACCGCGC | 7920 |
| | GCGACGTCGC CGGAGCGGTC GAGTTCTGGA CCGACCGGCT CGGGTTCTCC CGGGACTTCG | 7980 |
| | TGGAGGACGA CTTCGCCGGT GTGGTCCGGG ACGACGTGAC CCTGTTCATC AGCGCGGTCC | 8040 |
| | AGGACCAGGT GGTGCCGGAC AACACCCTGG CCTGGGTGTG GGTGCGCGGC CTGGACGAGC | 8100 |
| | TGTACGCCGA GTGGTCGGAG GTCGTGTCCA CGAACTTCCG GGACGCCTCC GGGCCGGCCA | 8160 |
| | TGACCGAGAT CGGCGAGCAG CCGTGGGGGC GGGAGTTCGC CCTGCGCGAC CCGGCCGGCA | 8220 |
| | ACTGCGTGCA CTTCGTGGCC GAGGAGCAGG ACTGACACGT GCTACGAGAT TTCGATTCCA | 8280 |
| | CCGCCGCCTT CTATGAAAGG TTGGGCTTCG GAATCGTTTT CCGGGACGCC GGCTGGATGA | 8340 |
| | TCCTCCAGCG CGGGGATCTC ATGCTGGAGT TCTTCGCCCA CCCCAACTTG TTTATTGCAG | 8400 |
| | CTTATAATGG TTACAAATAA AGCAATAGCA TCACAAATTT CACAAATAAA GCATTTTTTT | 8460 |
| | CACTGCATTC TAGTTGTGGT TTGTCCAAAC TCATCAATGT ATCTTATCAT GTCTGTATAC | 8520 |
| | CGTCGACCTC TAGCTAGAGC TTGGCGTAAT CATGGTCATA GCTGTTTCCT GTGTGAAATT | 8580 |
| | GTTATCCGCT CACAATTCCA CACAACATAC GAGCCGGAAG CATAAAGTGT AAAGCCTGGG | 8640 |
| | GTGCCTAATG AGTGAGCTAA CTCACATTAA TTGCGTTGCG CTCACTGCCC GCTTTCCAGT | 8700 |
| | CGGGAAACCT GTCGTGCCAG CTGCATTAAT GAATCGGCCA ACGCGCGGGG AGAGGCGGTT | 8760 |
| | TGCGTATTGG GCGCTCTTCC GCTTCCTCGC TCACTGACTC GCTGCGCTCG GTCGTTCGGC | 8820 |
| | TGCGGCGAGC GGTATCAGCT CACTCAAAGG CGGTAATACG GTTATCCACA GAATCAGGGG | 8880 |
| | ATAACGCAGG AAAGAACATG TGAGCAAAAG GCCAGCAAAA GGCCAGGAAC CGTAAAAAGG | 8940 |
| | CCGCGTTGCT GGCGETTTTC CATAGGCTCC GCCCCCCTGA CGAGCATCAC AAAAATCGAC | 9000 |
| | GCTCAAGTCA GAGGTGGCGA AACCCGACAG GACTATAAAG ATACCAGGCG TTTCCCCCTG | 9060 |
| | GAAGCTCCCT CGTGCGCTCT CCTGTTCCGA CCCTGCCGCT TACCGGATAC CTGTCCGCCT | 9120 |
| | TTCTCCCTTC GGGAAGCGTG GCGCTTTCTC ATAGCTCACG CTGTAGGTAT CTCAGTTCGG | 9180 |
| | TGTAGGTCGT TCGCTCCAAG CTGGGCTGTG TGCACGAACC CCCCGTTCAG CCCGACCGCT | 9240 |
| | GCGCCTTATC CGGTAACTAT CGTCTTGAGT CCAACCCGGT AAGACACGAC TTATCGCCAC | 9300 |
| | TGGCAGCAGC CACTGGTAAC AGGATTAGCA GAGCGAGGTA TGTAGGCGGT GCTACAGAGT | 9360 |
| | TCTTGAAGTG GTGGCCTAAC TACGGCTACA CTAGAAGAAC AGTATTTGGT ATCTGCGCTC | 9420 |
| | TGCTGAAGCC AGTTACCTTC GGAAAAAGAG TTGGTAGCTC TTGATCCGGC AAACAAACCA | 9480 |
| | CCGCTGGTAG CGGTGGTTTT TTTGTTTGCA AGCAGCAGAT TACGCGCAGA AAAAAAGGAT | 9540 |
| | CTCAAGAAGA TCCTTTGATC TTTTCTACGG GGTCTGACGC TCAGTGGAAC GAAAACTCAC | 9600 |
| | GTTAAGGGAT TTTGGTCATG AGATTATCAA AAAGGATCTT CACCTAGATC CTTTTAAATT | 9660 |
| | AAAAATGAAG TTTTAAATCA ATCTAAAGTA TATATGAGTA AACTTGGTCT GACAGTTACC | 9720 |
| | AATGCTTAAT CAGTGAGGCA CCTATCTCAG CGATCTGTCT ATTTCGTTCA TCCATAGTTG | 9780 |
| | CCTGACTCCC CGTCGTGTAG ATAACTACGA TACGGGAGGG CTTACCATCT GGCCCCAGTG | 9840 |
| | CTGCAATGAT ACCGCGAGAC CCACGCTCAC CGGCTCCAGA TTTATCAGCA ATAAACCAGC | 9900 |
| | CAGCCGGAAG GGCCGAGCGC AGAAGTGGTC CTGCAACTTT ATCCGCCTCC ATCCAGTCTA | 9960 |
| | TTAATTGTTG CCGGGAAGCT AGAGTAAGTA GTTCGCCAGT TAATAGTTTG CGCAACGTTG | 10020 |
| | TTGCCATTGC TACAGGCATC GTGGTGTCAC GCTCGTCGTT TGGTATGGCT TCATTCAGCT | 10080 |
| | CCGGTTCCCA ACGATCAAGG CGAGTTACAT GATCCCCCAT GTTGTGCAAA AAAGCGGTTA | 10140 |
| | GCTCCTTCGG TCCTCCGATC GTTGTCAGAA GTAAGTTGGC CGCAGTGTTA TCACTCATGG | 10200 |
| | TTATGGCAGC ACTGCATAAT TCTCTTACTG TCATGCCATC CGTAAGATGC TTTTCTGTGA | 10260 |
| | CTGGTGAGTA CTCAACCAAG TCATTCTGAG AATAGTGTAT GCGGCGACCG AGTTGCTCTT | 10320 |
| | GCCCGGCGTC AATACGGGAT AATACCGCGC CACATAGCAG AACTTTAAAA GTGCTCATCA | 10380 |
| | TTGGAAAACG TTCTTCGGGG CGAAAACTCT CAAGGATCTT ACCGCTGTTG AGATCCAGTT | 10440 |
| | CGATGTAACC CACTCGTGCA CCCAACTGAT CTTCAGCATC TTTTACTTTC ACCAGCGTTT | 10500 |
| | CTGGGTGAGC AAAAACAGGA AGGCAAAATG CCGCAAAAAA GGGAATAAGG GCGACACGGA | 10560 |
| | AATGTTGAAT ACTCATACTC TTCCTTTTTC AATATTATTG AAGCATTTAT CAGGGTTATT | 10620 |
| | GTCTCATGAG CGGATACATA TTTGAATGTA TTTAGAAAAA TAAACAAATA GGGGTTCCGC | 10680 |
| | GCACATTTCC CCGAAAAGTG CCACCTGAC | 10709 |
| SEQ ID NO: 711 | GTCGACGGAT CGGGAGATCT CCCGATCCCC TATGGTGCAC TCTCAGTACA ATCTGCTCTG | 60 |
| CCR19: ch | ATGCCGCATA GTTAAGCCAG TATCTGCTCC CTGCTTGTGT GTTGGAGGTC GCTGAGTAGT | 120 |
| TGFBRII-LTBR | GCGCGAGCAA AATTTAAGCT ACAACAAGGC AAGGCTTGAC CGACAATTGC ATGAAGAATC | 180 |
| | TGCTTAGGGT TAGGCGTTTT GCGCTGCTTC GCGATGTACG GGCCAGATAT ACGCGTTGAC | 240 |
| | ATTGATTATT GACTAGTTAT TAATAGTAAT CAATTACGGG GTCATTAGTT CATAGCCCAT | 300 |
| | ATATGGAGTT CCGCGTTACA TAACTTACGG TAAATGGCCC GCCTGGCTGA CCGCCCAACG | 360 |
| | ACCCCCGCCC ATTGACGTCA ATAATGACGT ATGTTCCCAT AGTAACGCCA ATAGGGACTT | 420 |
| | TCCATTGACG TCAATGGGTG GAGTATTTAC GGTAAACTGC CCACTTGGCA GTACATCAAG | 480 |
| | TGTATCATAT GCCAAGTACG CCCCCTATTG ACGTCAATGA CGGTAAATGG CCCGCCTGGC | 540 |
| | ATTATGCCCA GTACATGACC TTATGGGACT TTCCTACTTG GCAGTACATC TACGTATTAG | 600 |
| | TCATCGCTAT TACCATGGTG ATGCGGTTTT GGCAGTACAT CAATGGGCGT GGATAGCGGT | 660 |
| | TTGACTCACG GGGATTTCCA AGTCTCCACC CCATTGACGT CAATGGGAGT TTGTTTTGGC | 720 |
| | ACCAAAATCA ACGGGACTTT CCAAAATGTC GTAACAACTC CGCCCCATTG ACGCAAATGG | 780 |
| | GCGGTAGGCG TGTACGGTGG GAGGTCTATA TAAGCAGCGC GTTTTGCCTG TACTGGGTCT | 840 |
| | CTCTGGTTAG ACCAGATCTG AGCCTGGGAG CTCTCTGGCT AACTAGGGAA CCCACTGCTT | 900 |
| | AAGCCTCAAT AAAGCTTGCC TTGAGTGCTT CAAGTAGTGT GTGCCCGTCT GTTGTGTGAC | 960 |
| | TCTGGTAACT AGAGATCCCT CAGACCCTTT TAGTCAGTGT GGAAAATCTC TAGCAGTGGC | 1020 |
| | GCCCGAACAG GGACTTGAAA GCGAAAGGGA AACCAGAGGA GCTCTCTCGA CGCAGGACTC | 1080 |
| | GGCTTGCTGA AGCGCGCACG GCAAGAGGCG AGGGGCGGCG ACTGGTGAGT ACGCCAAAAA | 1140 |
| | TTTTGACTAG CGGAGGCTAG AAGGAGAGAG ATGGGTGCGA GAGCGTCAGT ATTAAGCGGG | 1200 |
| | GGAGAATTAG ATCGCGATGG GAAAAAATTC GGTTAAGGCC AGGGGGAAAG AAAAAATATA | 1260 |
| | AATTAAAACA TATAGTATGG GCAAGCAGGG AGCTAGAACG ATTCGCAGTT AATCCTGGCC | 1320 |
| | TGTTAGAAAC ATCAGAAGGC TGTAGACAAA TACTGGGACA GCTACAACCA TCCCTTCAGA | 1380 |
| | CAGGATCAGA AGAACTTAGA TCATTATATA ATACAGTAGC AACCCTCTAT TGTGTGCATC | 1440 |
| | AAAGGATAGA GATAAAAGAC ACCAAGGAAG CTTTAGACAA GATAGAGGAA GAGCAAAACA | 1500 |
| | AAAGTAAGAC CACCGCACAG CAAGCGGCCG CTGATCTTCA GACCTGGAGG AGGAGATATG | 1560 |
| | AGGGACAATT GGAGAAGTGA ATTATATAAA TATAAAGTAG TAAAAATTGA ACCATTAGGA | 1620 |
| | GTAGCACCCA CCAAGGCAAA GAGAAGAGTG GTGCAGAGAG AAAAAAGAGC AGTGGGAATA | 1680 |
| | GGAGCTTTGT TCCTTGGGTT CTTGGGAGCA GCAGGAAGCA CTATGGGCGC AGCGTCAATG | 1740 |
| | ACGCTGACGG TACAGGCCAG ACAATTATTG TCTGGTATAG TGCAGCAGCA GAACAATTTG | 1800 |
| | CTGAGGGCTA TTGAGGCGCA ACAGCATCTG TTGCAACTCA CAGTCTGGGG CATCAAGCAG | 1860 |
| | CTCCAGGCAA GAATCCTGGC TGTGGAAAGA TACCTAAAGG ATCAACAGCT CCTGGGGATT | 1920 |
| | TGGGGTTGCT CTGGAAAACT CATTTGCACC ACTGCTGTGC CTTGGAATGC TAGTTGGAGT | 1980 |
| | AATAAATCTC TGGAACAGAT TTGGAATCAC ACGACCTGGA TGGAGTGGGA CAGAGAAATT | 2040 |
| | AACAATTACA CAAGCTTAAT ACACTCCTTA ATTGAAGAAT CGCAAAACCA GCAAGAAAAG | 2100 |
| | AATGAACAAG AATTATTGGA ATTAGATAAA TGGGCAAGTT TGTGGAATTG GTTTAACATA | 2160 |
| | ACAAATTGGC TGTGGTATAT AAAATTATTC ATAATGATAG TAGGAGGCTT GGTAGGTTTA | 2220 |
| | AGAATAGTTT TTGCTGTACT TTCTATAGTG AATAGAGTTA GGCAGGGATA TTCACCATTA | 2280 |
| | TCGTTTCAGA CCCACCTCCC AACCCCGAGG GGACCCGACA GGCCCGAAGG AATAGAAGAA | 2340 |
| | GAAGGTGGAG AGAGAGACAG AGACAGATCC ATTCGATTAG TGAACGGATC GGCACTGCGT | 2400 |
| | GCGCCAATTC TGCAGACAAA TGGCAGTATT CATCCACAAT TTTAAAAGAA AAGGGGGGAT | 2460 |
| | TGGGGGGTAC AGTGCAGGGG AAAGAATAGT AGACATAATA GCAACAGACA TACAAACTAA | 2520 |
| | AGAATTACAA AAACAAATTA CAAAAATTCA AAATTTTCGG GTTTATTACA GGGACAGCAG | 2580 |
| | AGATCCAGTT TGGTTAATTA GCTAGCTAGG TCTTGAAAGG AGTGGGAATT GGCTCCGGTG | 2640 |
| | CCCGTCAGTG GGCAGAGCGC ACATCGCCCA CAGTCCCCGA GAAGTTGGGG GGAGGGGTCG | 2700 |
| | GCAATTGATC CGGTGCCTAG AGAAGGTGGC GCGGGGTAAA CTGGGAAAGT GATGTCGTGT | 2760 |
| | ACTGGCTCCG CCTTTTTCCC GAGGGTGGGG GAGAACCGTA TATAAGTGCA GTAGTCGCCG | 2820 |
| | TGAACGTTCT TTTTCGCAAC GGGTTTGCCG CCAGAACACA GGACCGGTTC TAGAGCGCTT | 2880 |
| | TAATTAAGCC ACCAEGGATT GGACCTGGAT CCTGTTCCTG GTGGCTGCGG CTACACGGGT | 2940 |
| | CCACTCACAG GTCACCGACA TTAACTCGAA GGGCCTGGAG CTGCGCAAGA CTGTGACGAC | 3000 |
| | CGTGGAGACC CAGAACCTGG AAGGTTTGCA CCACGATGGA CAGTTTTGTC ACAAGCCCTG | 3060 |
| | CCCGCCTGGT GAGCGGAAGG CCCGCGACTG TACCGTCAAC GGAGACGAGC CCGACTGCGT | 3120 |
| | GCCTTGCCAG GAGGGCAAAG AGTACACCGA CAAGGCCCAC TTTAGCTCCA AATGCCGTAG | 3180 |
| | GTGTCGCCTG TGCGACGAGG GCCACGGGCT GGAGGTGGAG ATCAACTGCA CTCGCACCCA | 3240 |
| | GAACACGAAA TGCAGATGTA AGCCTAACTT TTTCTGTAAT TCCACCGTTT GCGAGCACTG | 3300 |
| | TGATCCATGC ACCAAGTGCG AGCACGGTAT CATCAAGGAG TGCACTCTGA CCTCCAATAC | 3360 |
| | GAAGTGCAAG GAGGAGGGTT CCCGCAGCAA TCCACTGCCC CCCGAGATGT CTGGCACCAT | 3420 |
| | GCTGATGCTC GCGGTGCTGC TCCCTCTAGC GTTCTTCCTG CTGCTTGCCA CCGTATTCAG | 3480 |
| | CTGTATTTGG AAGAGCCACC CCTCACTGTG CCGTAAGCTC GGCTCTCTGC TGAAGCGCCG | 3540 |
| | ACCTCAGGGA GAGGGACCCA ACCCTGTGGC TGGGAGTTGG GAGCCGCCCA AGGCCCATCC | 3600 |
| | CTATTTCCCG GATCTTGTGC AGCCATTACT CCCCATCTCT GGTGACGTGT CCCCAGTGTC | 3660 |
| | GACCGGCCTT CCGGCCGCTC CGGTTCTAGA AGCAGGCGTG CCCCAACAGC AGTCCCCCTT | 3720 |
| | GGACCTGACT CGTGAGCCGC AGCTGGAGCC CGGGGAACAG TCCCAGGTGG CGCATGGCAC | 3780 |
| | CAACGGCATC CATGTCACAG GCGGCTCCAT GACCATCACC GGGAACATCT ACATATACAA | 3840 |
| | CGGTCCGGTG CTGGGTGGGC CTCCCGGCCC TGGGGACCTT CCCGCAACTC CGGAACCACC | 3900 |
| | CTACCCAATC CCTGAGGAGG GTGACCCGGG ACCCCCGGGC CTCTCTACCC CCCACCAGGA | 3960 |
| | GGACGGCAAA GCCTGGCACT TGGCCGAGAC CGAGCACTGT GGCGCTACTC CTAGCAACCG | 4020 |
| | CGGCCCGCGC AACCAGTTCA TTACTCATGA CTGAGAATTC CGCCCCCCCC CCCCCCCCCC | 4080 |
| | CTCTCCCTCC CCCCCCCCTA ACGTTACTGG CCGAAGCCGC TTGGAATAAG GCCGGTGTGC | 4140 |
| | GTTTGTCTAT ATGTTATTTT CCACCATATT GCCGTCTTTT GGCAATGTGA GGGCCCGGAA | 4200 |
| | ACCTGGCCCT GTCTTCTTGA CGAGCATTCC TAGGGGTCTT TCCCCTCTCG CCAAAGGAAT | 4260 |
| | GCAAGGTCTG TTGAATGTCG TGAAGGAAGC AGTTCCTCTG GAAGCTTCTT GAAGACAAAC | 4320 |
| | AACGTCTGTA GCGACCCTTT GCAGGCAGCG GAACCCCCCA CCTGGCGACA GGTGCCTCTG | 4380 |
| | CGGCCAAAAG CCACGTGTAT AAGATACACC TGCAAAGGCG GCACAACCCC AGTGCCACGT | 4440 |
| | TGTGAGTTGG ATAGTTGTGG AAAGAGTCAA ATGGCTCTCC TCAAGCGTAT TCAACAAGGG | 4500 |
| | GCTGAAGGAT GCCCAGAAGG TACCCCATTG TATGGGATCT GATCTGGGGC CTCGGTGCAC | 4560 |
| | ATGCTTTACA TGTGETTAGT CGAGGTTAAA AAAACGTCTA GGCCCCCCGA ACCACGGGGA | 4620 |
| | CGTGGTTTTC CTTTGAAAAA CACGATGATA AGCTTGATCA CGCGTGCCAC CATGAGCAAG | 4680 |
| | GGCGAGGAGC TGTTCACCGG GGTGGTGCCC ATCCTGGTCG AGCTGGACGG CGACGTAAAC | 4740 |
| | GGCCACAAGT TCAGCGTGTC CGGCGAGGGC GAGGGCGATG CCACCTACGG CAAGCTGACC | 4800 |
| | CTGAAGTTCA TCTGCACCAC CGGCAAGCTG CCCGTGCCCT GGCCCACCCT CGTGACCACC | 4860 |
| | CTGACCTACG GCGTGCAGTG CTTCAGCCGC TACCCCGACC ACATGAAGCA GCACGACTTC | 4920 |
| | TTCAAGTCCG CCATGCCCGA AGGCTACGTC CAGGAGCGCA CCATCTTCTT CAAGGACGAC | 4980 |
| | GGCAACTACA AGACCCGCGC CGAGGTGAAG TTCGAGGGCG ACACCCTGGT GAACCGCATC | 5040 |
| | GAGCTGAAGG GCATCGACTT CAAGGAGGAC GGCAACATCC TGGGGCACAA GCTGGAGTAC | 5100 |
| | AACTACAACA GCCACAACGT CTATATCATG GCCGACAAGC AGAAGAACGG CATCAAGGTG | 5160 |
| | AACTTCAAGA TCCGCCACAA CATCGAGGAC GGCAGCGTGC AGCTCGCCGA CCACTACCAG | 5220 |
| | CAGAACACCC CCATCGGCGA CGGCCCCGTG CTGCTGCCCG ACAACCACTA CCTGAGCACC | 5280 |
| | CAGTCCGCCC TGAGCAAAGA CCCCAACGAG AAGCGCGATC ACATGGTCCT GCTGGAGTTC | 5340 |
| | GTGACCGCCG CCGGGATCAC TCACGGCATG GACGAGCTGT ACAAGTGAGA ATTCGATATC | 5400 |
| | AAGCTTATCG GTAASCAACC TCTGGATTAC AAAATTTGTG AAAGATTGAC TGGTATTCTT | 5460 |
| | AACTATGTTG CTCCTTTTAC GCTATGTGGA TACGCTGCTT TAATGCCTTT GTATCATGCT | 5520 |
| | ATTGCTTCCC GTATGGCTTT CATTTTCTCC TCCTTGTATA AATCCTGGTT GCTGTCTCTT | 5580 |
| | TATGAGGAGT TGTGGCCCGT TGTCAGGCAA CGTGGCGTGG TGTGCACTGT GTTTGCTGAC | 5640 |
| | GCAACCCCCA CTGGETGGGG CATTGCCACC ACCTGTCAGC TCCTTTCCGG GACTTTCGCT | 5700 |
| | TTCCCCCTCC CTATTGCCAC GGCGGAACTC ATCGCCGCCT GCCTTGCCCG CTGCTGGACA | 5760 |
| | GGGGCTCGGC TGTTGGGCAC TGACAATTCC GTGGTGTTGT CGGGGAAATC ATCGTCCTTT | 5820 |
| | CCTTGGCTGC TCGCCTGTGT TGCCACCTGG ATTCTGCGCG GGACGTCCTT CTGCTACGTC | 5880 |
| | CCTTCGGCCC TCAATCCAGC GGACCTTCCT TCCCGCGGCC TGCTGCCGGC TCTGCGGCCT | 5940 |
| | CTTCCGCGTC TTCGCCTTCG CCCTCAGACG AGTOGGATCT CCCTTTGGGC CGCCTCCCCG | 6000 |
| | CATCGATACC GTCGACCTCG AGACCTAGAA AAACATGGAG CAATCACAAG TAGCAATACA | 6060 |
| | GCAGCTACCA ATGCTGATTG TGCCTGGCTA GAAGCACAAG AGGAGGAGGA GGTGGGTTTT | 6120 |
| | CCAGTCACAC CTCAGGTACC TTTAAGACCA ATGACTTACA AGGCAGCTGT AGATCTTAGC | 6180 |
| | CACTTTTTAA AAGAAAAGGG GGGACTGGAA GGGCTAATTC ACTCCCAACG AAGACAAGAT | 6240 |
| | ATCCTTGATC TGTGGATCTA CCACACACAA GGCTACTTCC CTGATTGGCA GAACTACACA | 6300 |
| | CCAGGGCCAG GGATCAGATA TCCACTGACC TTTGGATGGT GCTACAAGCT AGTACCAGTT | 6360 |
| | GAGCAAGAGA AGGTAGAAGA AGCCAATGAA GGAGAGAACA CCCGCTTGTT ACACCCTGTG | 6420 |
| | AGCCTGCATG GGATGGATGA CCCGGAGAGA GAAGTATTAG AGTGGAGGTT TGACAGCCGC | 6480 |
| | CTAGCATTTC ATCACATGGC CCGAGAGCTG CATCCGGACT GTACTGGGTC TCTCTGGTTA | 6540 |
| | GACCAGATCT GAGCCTGGGA GCTCTCTGGC TAACTAGGGA ACCCACTGCT TAAGCCTCAA | 6600 |
| | TAAAGCTTGC CTTGAGTGCT TCAAGTAGTG TGTGCCCGTC TGTTGTGTGA CTCTGGTAAC | 6660 |
| | TAGAGATCCC TCAGACCCTT TTAGTCAGTG TGGAAAATCT CTAGCAGGGC CCGTTTAAAC | 6720 |
| | CCGCTGATCA GCCTCGACTG TGCCTTCTAG TTGCCAGCCA TCTGTTGTTT GCCCCTCCCC | 6780 |
| | CGTGCCTTCC TTGACCCTGG AAGGTGCCAC TCCCACTGTC CTTTCCTAAT AAAATGAGGA | 6840 |
| | AATTGCATCG CATTGTCTGA GTAGGTGTCA TTCTATTCTG GGGGGTGGGG TGGGGCAGGA | 6900 |
| | CAGCAAGGGG GAGGATTGGG AAGACAATAG CAGGCATGCT GGGGATGCGG TGGGCTCTAT | 6960 |
| | GGCTTCTGAG GCGGAAAGAA CCAGCTGGGG CTCTAGGGGG TATCCCCACG CGCCCTGTAG | 7020 |
| | CGGCGCATTA AGCGCGGCGG GTGTGGTGGT TACGCGCAGC GTGACCGCTA CACTTGCCAG | 7080 |
| | CGCCCTAGCG CCCGCTCCTT TCGCTTTCTT CCCTTCCTTT CTCGCCACGT TCGCCGGCTT | 7140 |
| | TCCCCGTCAA GCTCTAAATC GGGGGCTCCC TTTAGGGTTC CGATTTAGTG CTTTACGGCA | 7200 |
| | CCTCGACCCC AAAAAACTTG ATTAGGGTGA TGGTTCACGT AGTGGGCCAT CGCCCTGATA | 7260 |
| | GACGGTTTTT CGCCCTTTGA CGTTGGAGTC CACGTTCTTT AATAGTGGAC TCTTGTTCCA | 7320 |
| | AACTGGAACA ACACTCAACC CTATCTCGGT CTATTCTTTT GATTTATAAG GGATTTTGCC | 7380 |
| | GATTTCGGCC TATTGGTTAA AAAATGAGCT GATTTAACAA AAATTTAACG CGAATTAATT | 7440 |
| | CTGTGGAATG TGTGTCAGTT AGGGTGTGGA AAGTCCCCAG GCTCCCCAGC AGGCAGAAGT | 7500 |
| | ATGCAAAGCA TGCATCTCAA TTAGTCAGCA ACCAGGTGTG GAAAGTCCCC AGGCTCCCCA | 7560 |
| | GCAGGCAGAA GTATGCAAAG CATGCATCTC AATTAGTCAG CAACCATAGT CCCGCCCCTA | 7620 |
| | ACTCCGCCCA TCCCGCCCCT AACTCCGCCC AGTTCCGCCC ATTCTCCGCC CCATGGCTGA | 7680 |
| | CTAATTTTTT TTATTTATGC AGAGGCCGAG GCCGCCTCTG CCTCTGAGCT ATTCCAGAAG | 7740 |
| | TAGTGAGGAG GCTTTTTTGG AGGCCTAGGC TTTTGCAAAA AGCTCCCGGG AGCTTGTATA | 7800 |
| | TCCATTTTCG GATCTGATCA GCACGTGTTG ACAATTAATC ATCGGCATAG TATATCGGCA | 7860 |
| | TAGTATAATA CGACAAGGTG AGGAACTAAA CCATGGCCAA GTTGACCAGT GCCGTTCCGG | 7920 |
| | TGCTCACCGC GCGCGACGTC GCCGGAGCGG TCGAGTTCTG GACCGACCGG CTCGGGTTCT | 7980 |
| | CCCGGGACTT CGTGGAGGAC GACTTCGCCG GTGTGGTCCG GGACGACGTG ACCCTGTTCA | 8040 |
| | TCAGCGCGGT CCAGGACCAG GTGGTGCCGG ACAACACCCT GGCCTGGGTG TGGGTGCGCG | 8100 |
| | GCCTGGACGA GCTGTACGCC GAGTGGTCGG AGGTCGTGTC CACGAACTTC CGGGACGCCT | 8160 |
| | CCGGGCCGGC CATGACCGAG ATCGGCGAGC AGCCGTGGGG GCGGGAGTTC GCCCTGCGCG | 8220 |
| | ACCCGGCCGG CAACTGCGTG CACTTCGTGG CCGAGGAGCA GGACTGACAC GTGCTACGAG | 8280 |
| | ATTTCGATTC CACCGCCGCC TTCTATGAAA GGTTGGGCTT CGGAATCGTT TTCCGGGACG | 8340 |
| | CCGGCTGGAT GATCCTCCAG CGCGGGGATC TCATGCTGGA GTTCTTCGCC CACCCCAACT | 8400 |
| | TGTTTATTGC AGCTTATAAT GGTTACAAAT AAAGCAATAG CATCACAAAT TTCACAAATA | 8460 |
| | AAGCATTTTT TTCACTGCAT TCTAGTTGTG GTTTGTCCAA ACTCATCAAT GTATCTTATC | 8520 |
| | ATGTCTGTAT ACCGTCGACC TCTAGCTAGA GCTTGGCGTA ATCATGGTCA TAGCTGTTTC | 8580 |
| | CTGTGTGAAA TTGTTATCCG CTCACAATTC CACACAACAT ACGAGCCGGA AGCATAAAGT | 8640 |
| | GTAAAGCCTG GGGTGCCTAA TGAGTGAGCT AACTCACATT AATTGCGTTG CGCTCACTGC | 8700 |
| | CCGCTTTCCA GTCGGGAAAC CTGTCGTGCC AGCTGCATTA ATGAATCGGC CAACGCGCGG | 8760 |
| | GGAGAGGCGG TTTGCGTATT GGGCGCTCTT CCGCTTCCTC GCTCACTGAC TCGCTGCGCT | 8820 |
| | CGGTCGTTCG GCTGCGGCGA GCGGTATCAG CTCACTCAAA GGCGGTAATA CGGTTATCCA | 8880 |
| | CAGAATCAGG GGATAACGCA GGAAAGAACA TGTGAGCAAA AGGCCAGCAA AAGGCCAGGA | 8940 |
| | ACCGTAAAAA GGCCGCGTTG CTGGCGTTTT TCCATAGGCT CCGCCCCCCT GACGAGCATC | 9000 |
| | ACAAAAATCG ACGCTCAAGT CAGAGGTGGC GAAACCCGAC AGGACTATAA AGATACCAGG | 9060 |
| | CGTTTCCCCC TGGAAGCTCC CTCGTGCGCT CTCCTGTTCC GACCCTGCCG CTTACCGGAT | 9120 |
| | ACCTGTCCGC CTTTCTCCCT TCGGGAAGCG TGGCGCTTTC TCATAGCTCA CGCTGTAGGT | 9180 |
| | ATCTCAGTTC GGTGTAGGTC GTTCGCTCCA AGCTGGGCTG TGTGCACGAA CCCCCCGTTC | 9240 |
| | AGCCCGACCG CTGCGCCTTA TCCGGTAACT ATCGTCTTGA GTCCAACCCG GTAAGACACG | 9300 |
| | ACTTATCGCC ACTGGCAGCA GCCACTGGTA ACAGGATTAG CAGAGCGAGG TATGTAGGCG | 9360 |
| | GTGCTACAGA GTTCTTGAAG TGGTGGCCTA ACTACGGCTA CACTAGAAGA ACAGTATTTG | 9420 |
| | GTATCTGCGC TCTGCTGAAG CCAGTTACCT TCGGAAAAAG AGTTGGTAGC TCTTGATCCG | 9480 |
| | GCAAACAAAC CACCGCTGGT AGCGGTGGTT TTTTTGTTTG CAAGCAGCAG ATTACGCGCA | 9540 |
| | GAAAAAAAGG ATCTCAAGAA GATCCTTTGA TCTTTTCTAC GGGGTCTGAC GCTCAGTGGA | 9600 |
| | ACGAAAACTC ACGTTAAGGG ATTTTGGTCA TGAGATTATC AAAAAGGATC TTCACCTAGA | 9660 |
| | TCCTTTTAAA TTAAAAATGA AGTTTTAAAT CAATCTAAAG TATATATGAG TAAACTTGGT | 9720 |
| | CTGACAGTTA CCAATGCTTA ATCAGTGAGG CACCTATCTC AGCGATCTGT CTATTTCGTT | 9780 |
| | CATCCATAGT TGCCTGACTC CCCGTCGTGT AGATAACTAC GATACGGGAG GGCTTACCAT | 9840 |
| | CTGGCCCCAG TGCTGCAATG ATACCGCGAG ACCCACGCTC ACCGGCTCCA GATTTATCAG | 9900 |
| | CAATAAACCA GCCAGCCGGA AGGGCCGAGC GCAGAAGTGG TCCTGCAACT TTATCCGCCT | 9960 |
| | CCATCCAGTC TATTAATTGT TGCCGGGAAG CTAGAGTAAG TAGTTCGCCA GTTAATAGTT | 10020 |
| | TGCGCAACGT TGTTGCCATT GCTACAGGCA TCGTGGTGTC ACGCTCGTCG TTTGGTATGG | 10080 |
| | CTTCATTCAG CTCCGGTTCC CAACGATCAA GGCGAGTTAC ATGATCCCCC ATGTTGTGCA | 10140 |
| | AAAAAGCGGT TAGCTCCTTC GGTCCTCCGA TCGTTGTCAG AAGTAAGTTG GCCGCAGTGT | 10200 |
| | TATCACTCAT GGTTATGGCA GCACTGCATA ATTCTCTTAC TGTCATGCCA TCCGTAAGAT | 10260 |
| | GCTTTTCTGT GACTGGTGAG TACTCAACCA AGTCATTCTG AGAATAGTGT ATGCGGCGAC | 10320 |
| | CGAGTTGCTC TTGCCCGGCG TCAATACGGG ATAATACCGC GCCACATAGC AGAACTTTAA | 10380 |
| | AAGTGCTCAT CATTGGAAAA CGTTCTTCGG GGCGAAAACT CTCAAGGATC TTACCGCTGT | 10440 |
| | TGAGATCCAG TTCGATGTAA CCCACTCGTG CACCCAACTG ATCTTCAGCA TCTTTTACTT | 10500 |
| | TCACCAGCGT TTCTGGGTGA GCAAAAACAG GAAGGCAAAA TGCCGCAAAA AAGGGAATAA | 10560 |
| | GGGCGACACG GAAATGTTGA ATACTCATAC TCTTCCTTTT TCAATATTAT TGAAGCATTT | 10620 |
| | ATCAGGGTTA TTGTCTCATG AGCGGATACA TATTTGAATG TATTTAGAAA AATAAACAAA | 10680 |
| | TAGGGGTTCC GCGCACATTT CCCCGAAAAG TGCCACCTGA C | 10721 |

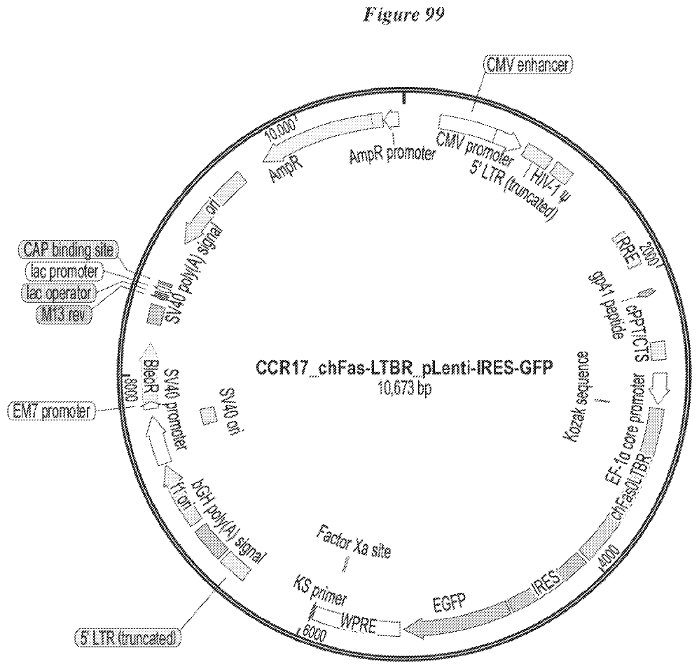

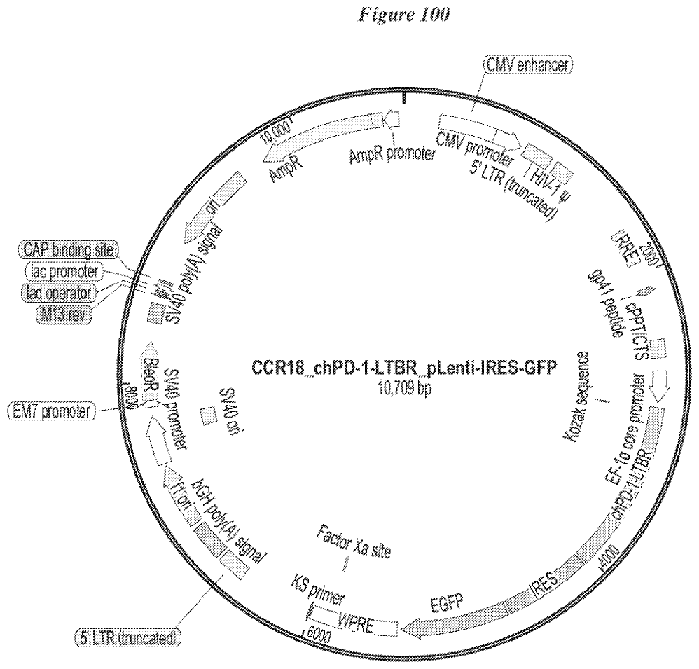

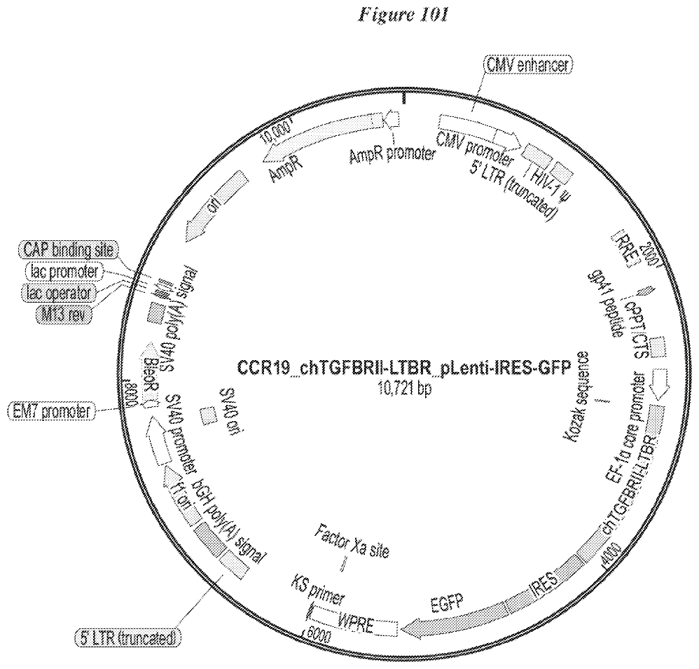

Vector maps for the exemplary vectors encoding the CCRs designated CCR17, CCR18, and CCR19, corresponding to SEQ ID NO:709 to SEQ ID NO:711, are presented in FIGS. 99 to 101. These vectors were used for the preparation of the lentiviral batches in this example.

The foregoing examples are also embodiments of the present invention. Further embodiments of present invention include the sequences of SEQ ID NO:703, SEQ ID NO:704, and SEQ ID NO:705, or conservative amino acid substitutions thereof, or fragments, variants, or derivatives thereof, or an amino acid sequence that is at least 99% identical to the sequence given in SEQ ID NO:703, SEQ ID NO:704, and SEQ ID NO:705, at least 98% identical to the sequence given in SEQ ID NO:703, SEQ ID NO:704, and SEQ ID NO:705, at least 97% identical to the sequence given in SEQ ID NO:703, SEQ ID NO:704, and SEQ ID NO:705, at least 96% identical to the sequence given in SEQ ID NO:703, SEQ ID NO:704, and SEQ ID NO:705, at least 95% identical to the sequence given in SEQ ID NO:703, SEQ ID NO:704, and SEQ ID NO:705, at least 90% identical to the sequence given in SEQ ID NO:703, SEQ ID NO:704, and SEQ ID NO:705, at least 85% identical to the sequence given in SEQ ID NO:703, SEQ ID NO:704, and SEQ ID NO:705, or at least 80% identical to the sequence given in SEQ ID NO:703, SEQ ID NO:704, and SEQ ID NO:705.

Further embodiments of present invention include the sequences of SEQ ID NO:706, SEQ ID NO:707, and SEQ ID NO:708, or fragments, variants, or derivatives thereof, or a nucleotide sequence that is at least 99% identical to the sequence given in SEQ ID NO:706, SEQ ID NO:707, and SEQ ID NO:708, at least 98% identical to the sequence given in SEQ ID NO:706, SEQ ID NO:707, and SEQ ID NO:708, at least 97% identical to the sequence given in SEQ ID NO:706, SEQ ID NO:707, and SEQ ID NO:708, at least 96% identical to the sequence given in SEQ ID NO:706, SEQ ID NO:707, and SEQ ID NO:708, at least 95% identical to the sequence given in SEQ ID NO:706, SEQ ID NO:707, and SEQ ID NO:708, at least 90% identical to the sequence given in SEQ ID NO:706, SEQ ID NO:707, and SEQ ID NO:708, at least 85% identical to the sequence given in SEQ ID NO:706, SEQ ID NO:707, and SEQ ID NO:708, or at least 80% identical to the sequence given in SEQ ID NO:706, SEQ ID NO:707, and SEQ ID NO:708.

Further embodiments of present invention include the sequences of SEQ ID NO:709, SEQ ID NO:710, and SEQ ID NO:711, or fragments, variants, or derivatives thereof, or a nucleotide sequence that is at least 99% identical to the sequence given in SEQ ID NO:709, SEQ ID NO:710, and SEQ ID NO:711, at least 98% identical to the sequence given in SEQ ID NO:709, SEQ ID NO:710, and SEQ ID NO:711, at least 97% identical to the sequence given in SEQ ID NO:709, SEQ ID NO:710, and SEQ ID NO:711, at least 96% identical to the sequence given in SEQ ID NO:709, SEQ ID NO:710, and SEQ ID NO:711, at least 95% identical to the sequence given in SEQ ID NO:709. SEQ ID NO:710, and SEQ ID NO:711, at least 90% identical to the sequence given in SEQ ID NO:709, SEQ ID NO:710, and SEQ ID NO:711, at least 85% identical to the sequence given in SEQ ID NO:709, SEQ ID NO:710, and SEQ ID NO:711, or at least 80% identical to the sequence given in SEQ ID NO:709, SEQ ID NO:710, and SEQ ID NO:711.

### Example 26: Chimeric Costimulatory Receptors with Anti-PD-L1 Extracellular Domains

Using the methods described above, additional CCRs may be prepared. These CCRs are designated CCR20, CCR21, CCR22, CCR23, CCR24, and CCR25 and use the anti-PD-L1 19H9 extracellular domain. Amino acid sequences for these CCRs are set forth in Table 96.

TABLE 96
Amino acid sequences of exemplary CCRs designated CCR20, CCR21, CCR22,
CCR23, CCR24, and CCR25.

| Identifier | Sequence (One-Letter Amino Acid Symbols) | |
|---|---|---|
| SEQ ID NO: 712 | MALPVTALLL PLALLLHAAR PNFMLTQPHS VSESLGKTVT ISCTGSSGSI ARKFVQWYQQ | 60 |
| CCR20: ch 19H9- | RPGSSPTTVI YENNQRPSGV SDRFSGSIGS SSNSASLTIS GLKTEDEADY YCQSYDSSNV | 120 |
| 4-1BB | VFGGGTKVTV LGGGGSGGGG SGGGGSGGGG SQVQLQESGG GLVKPGGSLR LSCAASGFTF | 180 |
| | SSYSMNWVRQ APGKGLEWVS GINTAGDTHY PESVKGRFTI SRDNARNSLN LQMNSLRAED | 240 |
| | TAVYYCVRER VEREYSGYDA FDIWGQGTTV TVSAPAPARE PGHSPQIISF FLALTSTALL | 300 |
| | FLLFFLTLRF SVVKRGRKKL LYIFKQPFMR PVQTTQEEDG CSCRFPEEEE GGCEL | 355 |
| SEQ ID NO: 713 | MALPVTALLL PLALLLHAAR PNFMLTQPHS VSESLGKTVT ISCTGSSGSI ARKFVQWYQQ | 60 |
| CCR21: ch 19H9- | RPGSSPTTVI YENNQRPSGV SDRFSGSIGS SSNSASLTIS GLKTEDEADY YCQSYDSSNV | 120 |
| LTBR | VFGGGTKVTV LGGGGSGGGG SGGGGSGGGG SQVQLQSSGG GLVKPGGSLR LSCAASGFTF | 180 |
| | SSYSMNWVRQ APGKGLEWVS GINTAGDTHY PESVKGRFTI SRDNARNSLN LQMNSLRAED | 240 |
| | TAVYYCVRER VEREYSGYDA FDIWGQGTTV TVSAPLPPEM SGTMLMLAVL LPLAFFLLLA | 300 |
| | TVFSCIWKSH PSLCRKLGSL LKRRPQGEGP NPVAGSWEPP KAHPYFPDLV QPLLPISGDV | 360 |
| | SPVSTGLPAA PVLEAGVPQQ QSPLDLTREP QLEPGEQSQV AHGTNGIHVT GGSMTITGNI | 420 |
| | YIYNGPVLGG PPGPGDLPAT PEPPYPIPEE GDPGPPGLST PHQEDGKAWH LAETEHCGAT | 480 |
| | PSNRGPRNQF ITHD | 494 |
| SEQ ID NO: 714 | MALPVTALLL PLALLLHAAR PNFMLTQPHS VSESLGKTVT ISCTGSSGSI ARKFVQWYQQ | 60 |
| CCR22: ch 19H9- | RPGSSPTTVI YENNQRPSGV SDRFSGSIGS SSNSASLTIS GLKTEDEADY YCQSYDSSNV | 120 |
| 4-1BB v2 | VFGGGTKVTV LGGGGSGGGG SGGGGSGGGG SQVQLQESGG GLVKPGGSLR LSCAASGFTF | 180 |
| | SSYSMNWVRQ APGKGLEWVS GINTAGDTHY PESVKGRFTI SRDNARNSLN LQMNSLRAED | 240 |
| | TAVYYCVRER VEREYSGYDA FDIWGQGTTV TVSAKPTTTP APRPPTPAPT IASQPLSLRP | 300 |
| | EACRPAAGGA VHTRGLDFAC DIYIWAPLAG TCGVLLLSLV ITLYKRGRKK LLYIFKQPFM | 360 |
| | RPVQTTQEED GCSCRFPEEE EGGCEL | 386 |
| SEQ ID NO: 715 | MALPVTALLL PLALLLHAAR PNFMLTQPHS VSESLGKTVT ISCTGSSGSI ARKFVQWYQQ | 60 |
| CCR23: ch 19H9- | RPGSSPTTVI YENNQRPSGV SDRFSGSIGS SSNSASLTIS GLKTEDEADY YCQSYDSSNV | 120 |
| LTBR v2 | VFGGGTKVTV LGGGGSGGGG SGGGGSGGGG SQVQLQESGG GLVKPGGSLR LSCAASGFTF | 180 |
| | SSYSMNWVRQ APGKGLEWVS GINTAGDTHY PESVKGRFTI SRDNARNSLN LQMNSLRAED | 240 |
| | TAVYYCVRER VEREYSGYDA FDIWGQGTTV TVSAKPTTTP APRPPTPAPT IASQPLSLRP | 300 |
| | EACRPAAGGA VHTRGLDFAC DIYIWAPLAG TCGVLLLSLV ITLYKSHPSL CRKLGSLLKR | 360 |
| | RPQGEGPNPV AGSWEPPKAH PYFPDLVQPL LPISGDVSPV STGLPAAPVL EAGVPQQQSP | 420 |
| | LDLTREPQLE PGEQSQVAHG TNGIHVTGGS MTITGNIYIY NGPVLGGPPG PGDLPATPEP | 480 |
| | PYPIPEEGDP GPPGLSTPHQ EDGKAWHLAE TEHCGATPSN RGPRNQFITH D | 531 |
| SEQ ID NO: 716 | MALPVTALLL PLALLLHAAR PNFMLTQPHS VSESLGKTVT ISCTGSSGSI ARKFVQWYQQ | 60 |
| CCR24: ch 19H9- | RPGSSPTTVI YENNQRPSGV SDRFSGSIGS SSNSASLTIS GLKTEDEADY YCQSYDSSNV | 120 |
| LTBR-4-1BB | VFGGGTKVTV LGGGGSGGGG SGGGGSGGGG SQVQLQESGG GLVKPGGSLR LSCAASGFTF | 180 |
| | SSYSMNWVRQ APGKGLEWVS GINTAGDTHY PESVKGRFTI SRDNARNSLN LQMNSLRAED | 240 |
| | TAVYYCVRER VEREYSGYDA FDIWGQGTTV TVSAKPTTTP APRPPTPAPT IASQPLSLRP | 300 |
| | EACRPAAGGA VHTRGLDFAC DIYIWAPLAG TCGVLLLSLV ITLYKSHPSL CRKLGSLLKR | 360 |
| | RPQGEGPNPV AGSWEPPKAH PYFPDLVQPL LPISGDVSPV STGLPAAPVL EAGVPQQQSP | 420 |
| | LDLTREPQLE PGEQSQVAHG TNGIHVTGGS MTITGNIYIY NGPVLGGPPG PGDLPATPEP | 480 |
| | PYPIPEEGDP GPPGLSTPHQ EDGKAWHLAE TEHCGATPSN RGPRNQFITH DKRGRKKLLY | 540 |
| | IFKQPFMRPV QTTQEEDGCS CRFPEEEEGG CEL | 573 |
| SEQ ID NO: 717 | MALPVTALLL PLALLLHAAR PNFMLTQPHS VSESLGKTVT ISCTGSSGSI ARKFVQWYQQ | 60 |
| CCR25: ch19H9-4- | RPGSSPTTVI YENNQRPSGV SDRFSGSIGS SSNSASLTIS GLKTEDEADY YCQSYDSSNV | 120 |
| 1BB-LTBR | VFGGGTKVTV LGGGGSGGGG SGGGGSGGGG SQVQLQESGG GLVKPGGSLR LSCAASGFTF | 180 |
| | SSYSMNWVRQ APGKGLEWVS GINTAGDTHY PESVKGRFTI SRDNARNSLN LQMNSLRAED | 240 |
| | TAVYYCVRER VEREYSGYDA FDIWGQGTTV TVSAKPTTTP APRPPTPAPT IASQPLSLRP | 300 |
| | EACRPAAGGA VHTRGLDFAC DIYIWAPLAG TCGVLLLSLV ITLYKRGRKK LLYIFKQPFM | 360 |
| | RPVQTTQEED GCSCRFPEEE EGGCELKSHP SLCRKLGSLL KRRPQGEGPN PVAGSWEPPK | 420 |
| | AHPYFPDLVQ PLLPISGDVS PVSTGLPAAP VLEAGVPQQQ SPLDLTREPQ LEPGEQSQVA | 480 |
| | HGTNGIHVTG GSMTITGNIY IYNGPVLGGP PGPGDLPATP EPPYPIPEEG DPGPPGLSTP | 540 |
| | HQEDGKAWHL AETEHCGATP SNRGPRNQFI THD | 573 |

Suitable, non-limiting embodiments of nucleotides encoding the CCRs prepared according to this example and useful as CCR constructs of the present invention are set forth in Table 97.

TABLE 97
Nucleotide sequences of exemplary CCRs designated CCR20, CCR21, CCR22,
CCR23, CCR24, and CCR25.

| Identifier | Sequence (One-Letter Nucleotide Symbols) | |
|---|---|---|
| SEQ ID NO: 718 | ATGGCGCTTC CGGTGACTGC TCTTCTCTTG CCCCTGGCTC TGCTGCTGCA CGCTGCCCGG | 60 |
| CCR20: ch 19H9- | CCCAACTTCA TGTTGACCCA GCCGCACTCC GTGTCGGAGA GCCTGGGCAA GACTGTCACG | 120 |
| 4-1BB | ATCTCATGCA CTGGATCTTC TGGGTCCATT GCTCGCAAAT TTGTGCAGTG GTACCAGCAG | 180 |
| | CGTCCCGGGT CTAGTCCGAC AACCGTGATC TACGAGAACA ACCAGAGACC ATCGGGCGTG | 240 |
| | TCCGACAGGT TTTCGGGATC TATCGGTAGC TCCTCCAACT CCGCTTCCCT CACCATTTCT | 300 |
| | GGTCTCAAGA CCGAGGACGA GGCTGATTAT TACTGCCAGA GCTACGACAG CAGCAACGTG | 360 |
| | GTGTTCGGTG GCGGCACCAA GGTCACTGTG CTGGGTGGAG GAGGCTCTGG CGGCGGAGGG | 420 |
| | TCCGGTGGCG GCGGCTCGGG GGGCGGCGGT TCGCAGGTCC AGCTGCAGGA GAGCGGTGGG | 480 |
| | GGGCTGGTGA AGCCTGGTGG TTCCCTACGG CTGTCTTGTG CCGCATCTGG CTTCACGTTC | 540 |
| | TCAAGCTATT CGATGAATTG GGTGCGCCAG GCCCCCGGCA AAGGACTGGA GTGGGTCTCC | 600 |
| | GGCATTAACA CGGCAGGTGA TACGCATTAC CCCGAGAGCG TGAAAGGCCG CTTCACCATC | 660 |
| | TCCCGCGACA ACGCGCGCAA CTCCCTCAAC CTGCAGATGA ACAGCCTTCG CGCCGAGGAC | 720 |
| | ACCGCCGTGT ACTACTGCGT GAGGGAGCGC GTGGAGCGCG AGTACTCCGG CTATGACGCC | 780 |
| | TTCGACATCT GGGGCCAGGG CACCACAGTC ACCGTATCTG CGCCGGCCCC TGCCCGAGAG | 840 |
| | CCAGGCCACT CCCCTCAGAT CATCTCCTTC TTCCTGGCCT TAACCTCCAC CGCGTTGCTG | 900 |
| | TTTCTGCTCT TTTTCCTGAC CCTGCGCTTC AGCGTTGTTA AGCGCGGCCG CAAGAAGCTG | 960 |
| | CTGTACATCT TCAAGCAACC CTTCATGCGT CCCGTGCAGA CTACCCAGGA GGAAGATGGG | 1020 |
| | TGCAGTTGTC GTTTTCCTGA AGAAGAGGAG GGGGGGTGCG AGCTG | 1065 |
| SEQ ID NO: 719 | ATGGCTCTGC CCGTCACTGC CCTCCTTCTT CCGCTCGCGC TGCTGCTGCA CGCTGCTCGG | 60 |
| CCR21: ch 19H9- | CCCAACTTTA TGCTAACCCA GCCGCACAGC GTCTCCGAGA GCCTGGGCAA AACCGTGACC | 120 |
| LTBR | ATCTCATGCA CTGGATCGAG TGGGTCCATC GCACGTAAAT TTGTGCAGTG GTACCAGCAG | 180 |
| | AGGCCCGGCA GCTCGCCAAC CACAGTGATT TACGAGAACA ACCAGCGTCC GTCCGGGGTT | 240 |
| | TCTGATCGTT TCTCGGGCTC TATCGGCTCC TCGAGCAACT CCGCCTCCCT GACGATCAGC | 300 |
| | GGACTCAAGA CCGAGGACGA GGCAGACTAT TACTGCCAGA GCTACGACTC TTCAAATGTG | 360 |
| | GTGTTCGGAG GAGGCACCAA GGTGACTGTC TTGGGTGGTG GGGGCTCCGG CGGCGGCGGC | 420 |
| | TCTGGAGGCG GCGGCTCTGG CGGCGGTGGC TCCCAGGTCC AGCTGCAGGA GAGCGGTGGG | 480 |
| | GGGCTGGTGA AGCCTGGCGG CTCGTTGCGC CTGTCATGCG CCGCTTCTGG CTTCACGTTT | 540 |
| | TCTTCGTACT CCATGAATTG GGTGCGCCAG GCCCCTGGTA AAGGTCTGGA GTGGGTCAGT | 600 |
| | GGAATTAACA CGGCCGGCGA CACCCACTAC CCTGAGTCTG TAAAGGGTCG CTTCACCATC | 660 |
| | TCTCGCGACA ACGCCAGAAA TTCCCTCAAC CTGCAGATGA ACTCCCTGCG CGCAGAGGAT | 720 |
| | ACTGCCGTGT ACTACTGCGT GAGGGAGCGC GTGGAGCGGG AGTACTCCGG CTACGACGCC | 780 |
| | TTCGACATCT GGGGCCAAGG CACCACCGTG ACTGTTTCCG CCCCCCTGCC TCCCGAGATG | 840 |
| | TCCGGGACGA TGCTGATGCT GGCTGTGCTG CTGCCGCTAG CGTTCTTCCT GTTGCTGGCG | 900 |
| | ACTGTCTTTA GCTGTATTTG GAAGTCCCAC CCCAGTTTGT GCCGAAAGCT GGGCTCCCTG | 960 |
| | CTGAAGCGCC GCCCTCAGGG GGAGGGCCCA AATCCAGTGG CGGGCAGTTG GGAGCCCCCA | 1020 |
| | AAGGCCCATC CCTATTTCCC CGACCTGGTG CAGCCGTTAC TGCCCATCTC CGGGGACGTG | 1080 |
| | TCCCCAGTTT CGACCGGTCT CCCTGCCGCA CCTGTCCTGG AGGCCGGCGT GCCTCAACAG | 1140 |
| | CAGAGCCCTT TGGACCTGAC CCGCGAACCT CAGCTTGAGC CAGGGGAACA GTCACAGGTA | 1200 |
| | GCGCATGGAA CCAACGGCAT CCACGTCACC GGAGGTTCTA TGACCATTAC AGGCAACATC | 1260 |
| | TACATATACA ACGGTCCGGT GCTAGGAGGT CCGCCAGGGC CGGGTGATCT CCCCGCCACC | 1320 |
| | CCAGAACCCC CCTATCCCAT CCCGGAGGAG GGCGACCCGG GCCCCCCCGG GCTGTCCACC | 1380 |
| | CCCCACCAGG AAGACGGCAA GGCGTGGCAC TTAGCCGAGA CTGAGCACTG TGGCGCCACA | 1440 |
| | CCTTCTAACC GCGGCCCGCG CAACCAGTTC ATCACCCATG ACTAA | 1485 |
| SEQ ID NO: 720 | ATGGCCTTGC CGGTGACTGC ACTCCTGCTG CCTCTGGCGC TGCTCTTACA CGCCGCACGT | 60 |
| CCR22: ch 19H9- | CCCAACTTTA TGCTGACCCA GCCGCATTCC GTGTCGGAGA GCCTGGGCAA AACTGTGACC | 120 |
| 4-1BB v2 | ATCTCGTGTA CGGGGTCGTC CGGGAGCATC GCTCGCAAAT TTGTGCAGTG GTACCAGCAG | 180 |
| | CGTCCGGGCT CTTCCCCCAC CACAGTAATT TACGAGAACA ACCAGCGGCC CTCTGGCGTT | 240 |
| | AGTGACAGGT TTTCAGGCTC TATCGGCAGC AGCAGCAACT CCGCCTCCCT AACTATCTCT | 300 |
| | GGTCTGAAGA CCGAGGACGA GGCGGACTAT TACTGTCAGA GCTATGATTC TTCCAATGTG | 360 |
| | GTGTTCGGAG GAGGGACTAA GGTGACGGTG CTGGGTGGTG GTGGGAGCGG AGGTGGCGGA | 420 |
| | TCCGGCGGGG GTGGCTCCGG TGGAGGCGGC TCCCAGGTCC AGTTGCAGGA GAGCGGCGGT | 480 |
| | GGCCTGGTGA AGCCCGGCGG CTCGTTGCGC CTGTCCTGTG CTGCTTCTGG CTTCACATTC | 540 |
| | AGTTCGTACT CCATGAACTG GGTCCGCCAG GCCCCCGGAA AAGGCCTGGA GTGGGTGTCC | 600 |
| | GGCATTAACA CGGCCGGGGA CACCCACTAC CCTGAATCTG TCAAGGGCCG CTTCACCATT | 660 |
| | TCCCGCGACA ACGCGAGGAA CTCCCTCAAC CTGCAGATGA ACTCCCTGCG CGCCGAGGAT | 720 |
| | ACCGCCGTGT ACTACTGCGT GCGAGAACGC GTGGAGCGCG AGTACTCAGG CTACGACGCA | 780 |
| | TTCGACATAT GGGGCCAGGG CACCACCGTC ACCGTGTCCG CCAAGCCAAC TACAACCCCC | 840 |
| | GCCCCGAGAC CCCCAACCCC TGCGCCAACC ATCGCTTCCC AACCCCTGTC TCTGCGGCCT | 900 |
| | GAGGCCTGCC GACCAGCGGC TGGCGGTGCT GTCCACACTC GCGGTTTGGA TTTCGCTTGC | 960 |
| | GACATCTACA TCTGGGCGCC TCTCGCCGGC ACCTGCGGGG TTCTTCTGCT GAGCCTTGTC | 1020 |
| | ATCACCCTTT ACAAGCGCGG ACGCAAGAAG CTGCTTTATA TCTTCAAGCA GCCCTTCATG | 1080 |
| | CGTCCTGTGC AGACGACCCA GGAGGAGGAC GGCTGTTCAT GCCGTTTCCC GGAAGAGGAG | 1140 |
| | GAGGGCGGGT GCGAGCTG | 1158 |
| SEQ ID NO: 721 | ATGGCACTGC CTGTGACGGC TCTGTTGCTG CCGCTCGCTC TTCTGCTACA CGCCGCTCGC | 60 |
| CCR23: ch 19H9- | CCCAACTTTA TGCTCACCCA GCCTCATTCC GTGTCGGAGT CATTAGGAAA AACCGTAACA | 120 |
| LTBR v2 | ATCTCTTGCA CTGGCTCATC TGGTTCCATT GCACGCAAAT TTGTGCAGTG GTACCAGCAG | 180 |
| | CGGCCAGGTA GCAGCCCAAC CACTGTCATC TACGAGAACA ACCAGCGTCC TTCGGGGGTG | 240 |
| | AGCGACAGGT TCTCGGGTTC TATCGGCTCG TCCTCCAACT CCGCTTCCCT GACAATTAGC | 300 |
| | GGGCTGAAGA CTGAGGACGA GGCGGACTAC TACTGTCAGA GCTACGATTC TTCCAACGTG | 360 |
| | GTGTTCGGCG GGGGCACCAA GGTGACTGTG CTGGGAGGAG GCGGATCTGG TGGCGGCGGT | 420 |
| | AGCGGTGGGG GCGGTTCAGG CGGTGGCGGC TCCCAGGTGC AGCTGCAGGA GTCTGGCGGA | 480 |
| | GGCCTCGTGA AGCCCGGTGG TTCTCTGAGA TTGAGTTGTG CCGCGTCGGG CTTCACCTTT | 540 |
| | AGCTCTTACT CCATGAATTG GGTCCGCCAG GCTCCCGGCA AGGGCCTTGA GTGGGTGAGC | 600 |
| | GGCATTAATA CGGCCGGGGA TACCCACTAC CCTGAGAGCG TTAAAGGCCG CTTCACCATC | 660 |
| | TCGCGAGACA ACGCGCGCAA CTCCCTCAAC CTGCAGATGA ACTCTCTGCG CGCTGAAGAC | 720 |
| | ACCGCCGTGT ACTACTGCGT GAGGGAGCGG GTGGAGCGTG AGTACTCCGG CTACGACGCC | 780 |
| | TTCGACATAT GGGGCCAAGG CACCACCGTC ACTGTCTCCG CCAAGCCCAC CACCACTCCC | 840 |
| | GCGCCACGCC CGCCTACACC CGCCCCCACG ATCGCCTCTC AGCCGCTGAG CCTCCGGCCG | 900 |
| | GAGGCCTGCC GTCCGGCCGC AGGCGGAGCC GTGCACACAC GCGGCTTGGA CTTCGCATGC | 960 |
| | GACATTTACA TCTGGGCCCC CCTGGCCGGC ACCTGCGGGG TGCTGCTTCT TTCGCTGGTG | 1020 |
| | ATCACCCTGT ACAAGAGCCA CCCTTCTCTG TGCCGCAAGC TGGGCTCCCT GTTAAAGCGC | 1080 |
| | CGTCCTCAGG GCGAGGGCCC CAATCCAGTG GCTGGGAGTT GGGAACCCCC CAAGGCGCAC | 1140 |
| | CCCTATTTCC CCGACCTGGT CCAGCCCCTC CTGCCCATCT CCGGAGACGT GTCCCCGGTA | 1200 |
| | TCCACCGGTC TGCCTGCTGC TCCGGTCCTG GAGGCCGGCG TGCCGCAACA GCAGAGCCCT | 1260 |
| | CTTGACCTGA CCCGCGAGCC ACAGCTCGAG CCCGGGGAAC AGTCCCAGGT CGCGCATGGA | 1320 |
| | ACCAACGGCA TCCACGTTAC CGGAGGTAGT ATGACTATCA CCGGCAACAT CTATATTTAC | 1380 |
| | AATGGCCCAG TTTTGGGTGG GCCCCCTGGC CCTGGGGATC TCCCAGCGAC GCCCGAACCG | 1440 |
| | CCCTATCCGA TCCCGGAGGA GGGCGATCCA GGACCCCCCG GCCTGTCCAC CCCTCACCAG | 1500 |
| | GAGGACGGCA AGGCCTGGCA CCTGGCCGAG ACCGAGCACT GTGGGGCCAC GCCGTCCAAC | 1560 |
| | CGCGGTCCCC GCAACCAGTT CATCACTCAT GAC | 1593 |
| SEQ ID NO: 722 | ATGGCCCTGC CCGTCACAGC ACTTCTCCTT CCCCTGGCGC TCCTGCTACA CGCCGCTCGC | 60 |
| CCR24: ch 19H9- | CCCAACTTTA TGCTCACCCA GCCTCATTCT GTTTCCGAGA GTTTGGGCAA AACCGTCACC | 120 |
| LTBR-4-1BB | ATTTCCTGCA CTGGTTCCTC CGGATCTATC GCGCGCAAAT TTGTCCAGTG GTACCAGCAG | 180 |
| | AGACCGGGCT CCAGCCCTAC CACCGTGATT TACGAGAACA ACCAGAGGCC CTCCGGTGTG | 240 |
| | AGCGATCGGT TTTCCGGTTC CATCGGTTCG AGTTCGAATA GCGCATCCCT GACTATCTCA | 300 |
| | GGGCTGAAGA CTGAAGACGA GGCGGACTAT TACTGCCAAA GCTACGATTC TTCCAACGTG | 360 |
| | GTGTTCGGCG GCGGGACCAA GGTGACCGTG TTGGGCGGCG GAGGATCTGG AGGCGGCGGT | 420 |
| | TCTGGGGGTG GGGGCAGCGG CGGTGGGGGC TCCCAGGTGC AGTTGCAGGA GAGCGGGGGC | 480 |
| | GGGCTTGTGA AGCCCGGCGG CTCGCTGCGC CTGTCATGTG CTGCTTCCGG CTTCACATTC | 540 |
| | TCGAGTTACT CCATGAACTG GGTGCGACAG GCCCCAGGCA AGGGCCTAGA GTGGGTCAGC | 600 |
| | GGAATTAACA CGGCCGGCGA CACACACTAC CCCGAGAGCG TTAAGGGACG CTTCACCATC | 660 |
| | TCTCGTGACA ACGCTCGCAA CTCCCTCAAC CTGCAGATGA ACTCCCTGCG AGCAGAGGAC | 720 |
| | ACCGCCGTGT ACTACTGCGT GCGGGAGCGC GTGGAGCGTG AGTACAGCGG ATACGACGCC | 780 |
| | TTCGACATAT GGGGCCAGGG CACAACCGTT ACCGTGTCCG CCAAGCCTAC GACCACTCCC | 840 |
| | GCTCCGCGGC CGCCTACCCC TGCCCCAACC ATCGCCAGCC AGCCACTTTC GCTGAGACCA | 900 |
| | GAAGCGTGCC GTCCGGCCGC AGGTGGCGCT GTCCACACTC GCGGCCTCGA CTTTGCCTGC | 960 |
| | GACATCTACA TTTGGGCGCC TTTGGCTGGC ACCTGCGGGG TGCTGCTGCT GAGCCTGGTG | 1020 |
| | ATCACCCTGT ACAAGAGTCA CCCCTCTCTG TGCCGCAAAC TGGGCTCCCT GCTCAAGCGC | 1080 |
| | CGCCCGCAAG GCGAGGGCCC CAATCCAGTG GCTGGCAGTT GGGAGCCACC CAAGGCTCAC | 1140 |
| | CCCTATTTCC CCGACCTGGT GCAGCCACTG CTGCCTATCT CCGGTGATGT ATCTCCGGTG | 1200 |
| | TCTACAGGTT TGCCTGCTGC CCCTGTCTTA GAGGCCGGCG TCCCTCAGCA GCAGAGCCCC | 1260 |
| | TTGGATCTGA CCAGGGAGCC CCAGCTTGAG CCAGGGGAAC AGTCACAGGT CGCGCATGGA | 1320 |
| | ACCAACGGCA TCCACGTAAC CGGCGGATCT ATGACGATTA CCGGCAACAT CTATATCTAC | 1380 |
| | AACGGACCCG TGCTGGGTGG GCCCCCGGGA CCCGGGGACC TGCCGGCCAC CCCCGAACCA | 1440 |
| | CCCTATCCCA TCCCTGAGGA GGGCGACCCG GGTCCCCCGG GCCTATCCAC CCCTCACCAG | 1500 |
| | GAGGATGGCA AGGCGTGGCA CCTGGCTGAG ACCGAGCACT GTGGCGCCAC GCCATCGAAC | 1560 |
| | CGCGGCCCGC GCAACCAGTT CATCACTCAT GACAAACGTG GCAGGAAGAA GCTGCTGTAC | 1620 |
| | ATCTTCAAGC AGCCGTTCAT GCGCCCTGTG CAGACGACCC AGGAGGAGGA CGGGTGTTCT | 1680 |
| | TGTCGCTTCC CGGAGGAGGA AGAGGGCGGG TGCGAGCTG | 1719 |
| SEQ ID NO: 723 | ATGGCCCTGC CCGTCACGGC CTTACTCCTG CCACTGGCGC TGCTGTTGCA CGCCGCGCGC | 60 |
| CCR25: ch 19H9- | CCTAACTTTA TGCTGACCCA GCCTCATAGC GTGTCCGAGA GCCTGGGTAA AACGGTCACC | 120 |
| 4-1BB-LTBR | ATCAGTTGCA CTGGCTCGTC TGGAAGCATC GCCCGCAAAT TTGTGCAGTG GTACCAGCAG | 180 |
| | CGCCCGGGCA GCTCGCCGAC CACAGTTATT TACGAGAACA ACCAGAGGCC CTCCGGCGTC | 240 |
| | TCCGACAGGT TTTCAGGCTC CATCGGTAGC TCCTCAAATT CCGCTTCCCT AACTATCTCT | 300 |
| | GGCCTGAAGA CTGAAGACGA GGCGGACTAT TACTGCCAGA GTTACGATTC TTCCAACGTG | 360 |
| | GTGTTCGGAG GTGGCACCAA GGTGACCGTG CTCGGGGGCG GTGGCTCGGG CGGGGGCGGT | 420 |
| | TCCGGTGGCG GCGGCTCTGG TGGGGGGGGC AGCCAGGTCC AATTGCAGGA GAGTGGGGGT | 480 |
| | GGCCTGGTCA AGCCCGGCGG CTCCCTCCGC CTGTCTTGCG CTGCTTCTGG CTTCACGTTC | 540 |
| | TCGTCCTACT CTATGAATTG GGTCCGCCAG GCCCCGGGCA AAGGCCTCGA GTGGGTGTCC | 600 |
| | GGAATTAACA CGGCCGGGGA CACCCACTAC CCCGAGTCCG TAAAGGGGCG ATTCACCATA | 660 |
| | TCACGCGACA ACGCTCGCAA CAGCCTCAAC CTGCAGATGA ACTCTCTGCG TGCCGAGGAC | 720 |
| | ACCGCCGTGT ACTACTGCGT GCGCGAGCGC GTGGAGCGGG AGTACTCCGG CTACGACGCC | 780 |
| | TTCGACATTT GGGGCCAGGG AACTACCGTC ACAGTCAGCG CCAAGCCTAC CACAACCCCC | 840 |
| | GCGCCTCGGC CCCCGACTCC TGCTCCCACC ATCGCTAGCC AGCCACTGTC CCTGCGCCCC | 900 |
| | GAGGCATGCC GACCAGCAGC AGGCGGCGCC GTGCACACAA GAGGATTGGA TTTTGCTTGC | 960 |
| | GACATCTACA TCTGGGCCCC GCTGGCGGGC ACCTGCGGGG TGCTACTGCT CTCGCTGGTG | 1020 |
| | ATTACCCTGT ACAAGCGTGG CCGCAAGAAG CTGCTTTACA TCTTCAAGCA GCCCTTCATG | 1080 |
| | CGCCCTGTGC AGACGACCCA GGAGGAGGAT GGATGTTCTT GTCGTTTCCC TGAAGAAGAG | 1140 |
| | GAGGGCGGGT GCGAGTTGAA ATCCCACCCC TCGCTGTGCC GCAAGCTGGG TAGCCTCCTA | 1200 |
| | AAGCGTCGCC CTCAGGGCGA GGGCCCTAAT CCAGTGGCTG GAAGTTGGGA GCCACCCAAG | 1260 |
| | GCGCACCCCT ATTTCCCCGA CTTGGTGCAG CCCCTGCTGC CCATCTCTGG TGATGTAAGC | 1320 |
| | CCGGTTTCCA CCGGCCTTCC TGCAGCGCCA GTTCTGGAGG CTGGCGTGCC ACAACAGCAG | 1380 |
| | TCGCCTCTCG ACCTGACTAG GGAGCCCCAG CTGGAGCCAG GGGAACAGTC ACAGGTGGCG | 1440 |
| | CATGGAACCA ACGGCATCCA CGTCACCGGT GGCTCCATGA CCATCACTGG CAACATCTAT | 1500 |
| | ATCTACAACG GCCCCGTGCT GGGCGGCCCA CCTGGACCGG GAGATCTGCC TGCCACCCCC | 1560 |
| | GAACCCCCAT ATCCGATCCC GGAGGAGGGT GACCCGGGAC CCCCGGGGCT TTCCACCCCG | 1620 |
| | CACCAGGAGG ACGGCAAGGC CTGGCACTTG GCCGAGACCG AGCACTGTGG TGCTACTCCC | 1680 |
| | TCTAACCGGG GTCCCCGCAA CCAGTTCATC ACGCATGAC | 1719 |

Vectors and procedures analogous to those described in Examples 25 and 26 may be employed for the preparation of TILs expressing CCR20 to CCR25.

The foregoing examples are also embodiments of the present invention. Further embodiments of present invention include the sequences of SEQ ID NO:712, SEQ ID NO:713, SEQ ID NO:714, SEQ ID NO:715, SEQ ID NO:716, and SEQ ID NO:717, or conservative amino acid substitutions thereof, or fragments, variants, or derivatives thereof, or an amino acid sequence that is at least 99% identical to the sequence given in SEQ ID NO:712, SEQ ID NO:713, SEQ ID NO:714, SEQ ID NO:715, SEQ ID NO:716, and SEQ ID NO:717, at least 98% identical to the sequence given in SEQ ID NO:712, SEQ ID NO:713, SEQ ID NO:714, SEQ ID NO:715, SEQ ID NO:716, and SEQ ID NO:717, at least 97% identical to the sequence given in SEQ ID NO:712, SEQ ID NO:713, SEQ ID NO:714, SEQ ID NO:715, SEQ ID NO:716, and SEQ ID NO:717, at least 96% identical to the sequence given in SEQ ID NO:712, SEQ ID NO:713, SEQ ID NO:714, SEQ ID NO:715, SEQ ID NO:716, and SEQ ID NO:717, at least 95% identical to the sequence given in SEQ ID NO:712, SEQ ID NO:713, SEQ ID NO:714, SEQ ID NO:715, SEQ ID NO:716, and SEQ ID NO:717, at least 90% identical to the sequence given in SEQ ID NO:712, SEQ ID NO:713, SEQ ID NO:714, SEQ ID NO:715, SEQ ID NO:716, and SEQ ID NO:717, at least 85% identical to the sequence given in SEQ ID NO:712, SEQ ID NO:713, SEQ ID NO:714, SEQ ID NO:715, SEQ ID NO:716, and SEQ ID NO:717, or at least 80% identical to the sequence given in SEQ ID NO:712, SEQ ID NO:713, SEQ ID NO:714, SEQ ID NO:715, SEQ ID NO:716, and SEQ ID NO:717.

Further embodiments of present invention include the sequences of SEQ ID NO:718, SEQ ID NO:719, SEQ ID NO:720, SEQ ID NO:721, SEQ ID NO:722, and SEQ ID NO:723, or fragments, variants, or derivatives thereof, or a nucleotide sequence that is at least 99% identical to the sequence given in SEQ ID NO:718, SEQ ID NO:719, SEQ ID NO:720, SEQ ID NO:721, SEQ ID NO:722, and SEQ ID NO:723, at least 98% identical to the sequence given in S SEQ ID NO:718, SEQ ID NO:719, SEQ ID NO:720, SEQ ID NO:721, SEQ ID NO:722, and SEQ ID NO:723, at least 97% identical to the sequence given in SEQ ID NO:718, SEQ ID NO:719, SEQ ID NO:720, SEQ ID NO:721, SEQ ID NO:722, and SEQ ID NO:723, at least 96% identical to the sequence given in SEQ ID NO:718, SEQ ID NO:719, SEQ ID NO:720, SEQ ID NO:721, SEQ ID NO:722, and SEQ ID NO:723, at least 95% identical to the sequence given in SEQ ID NO:718, SEQ ID NO:719, SEQ ID NO:720, SEQ ID NO:721, SEQ ID NO:722, and SEQ ID NO:723, at least 90% identical to the sequence given in SEQ ID NO:718, SEQ ID NO:719, SEQ ID NO:720, SEQ ID NO:721, SEQ ID NO:722, and SEQ ID NO:723, at least 85% identical to the sequence given in SEQ ID NO:718, SEQ ID NO:719, SEQ ID NO:720, SEQ ID NO:721, SEQ ID NO:722, and SEQ ID NO:723, or at least 80% identical to the sequence given in SEQ ID NO:718, SEQ ID NO:719, SEQ ID NO:720, SEQ ID NO:721, SEQ ID NO:722, and SEQ ID NO:723.

The examples set forth above are provided to give those of ordinary skill in the art a complete disclosure and description of how to make and use the embodiments of the compositions, processes, assays, systems, and methods of the invention, and are not intended to limit the scope of what the inventors regard as their invention. Modifications of the above-described modes for carrying out the invention that are obvious to persons of skill in the art are intended to be within the scope of the following claims. All patents, patent applications, and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chimeric costimulatory receptor (CCR), wherein the CCR comprises:
i. An extracellular domain,
ii. A hinge domain,
iii. A transmembrane domain, and
iv. At least one intracellular domain.

2. The method of claim 1, wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the CCR;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient.

3. The method of any one of claims 1 to 2, wherein the extracellular domain comprises an scFv binding domain.

4. The method of claim 3, wherein the scFv binding domain binds to a protein selected from the group consisting of DLL3, CD19, CD20, CD22, CD24, CD33, CD38, CD39, CD73, CD123, CD138, CD228, LRRC15, CEA, FRα, EPCAM, PD-L1, PSMA, gp100, MUC1, MCSP, EGFR, GD2, TROP-2, GPC3, MICA, MICB, VISTA, ULBP, HER2, MCM5, FAP, 5T4, LFA-1, B7-H3, IL-13Rα2, FAS, TGFβ, TGFβRII, and MUC16.

5. The method of any one of claims 1 to 2, wherein the extracellular domain is selected from the group consisting of a DDL3 domain, a PD-1 domain, a FAS domain, and a TGFβRII domain.

6. The method of any one of claims 1 to 5, wherein the intracellular domain is selected from the group consisting of CD28, CD134 (OX40), CD278 (ICOS), CD137 (4-1BB), CD27, CD40L, STAT3, IL-2Rβ, IL-2Rγ, IL-18R1, IL-18RAP, IL-7Rα, IL-12R1, IL-12R2, IL-15Rα, IL-21R, LTBR, and combinations thereof.

7. The method of any one of claims 1 to 6, wherein the transmembrane domain is selected from the group consisting of the transmembrane region of CD3α, CD3β, CDζ, CD3ε, CD4, CD5, CD8a, CD9, CD16, CD22, CD27, CD28, CD33, CD37, CD45, CD64, CD80, CD86, CD134, CD137, CD154, IgG1, IgG4, IgD, IL-2Rα, IL-2Rβ, IL-2Rγ, and CD40L.

8. The method of any one of claims 2 to 7, wherein step (d) further comprises genetically modifying TILs using a lentivirus to express the CCR.

9. The method of any one of claims 1 to 8, wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), SOCS1, ANKRD11, BCOR, and combinations thereof.

10. The method of any one of claims 2 to 9, wherein the cancer is a solid tumor cancer treated by administration of TILs.

11. The method of claim 10, wherein the cancer is selected from the group consisting of sarcoma, pancreatic cancer, liver cancer, glioblastoma, gastrointestinal cancer, melanoma, ovarian cancer, endometrial cancer, thyroid cancer, colorectal cancer, cervical cancer, lung cancer, non-small-cell lung cancer, small-cell lung cancer, mesothelioma, bladder cancer, breast cancer, cancer caused by human papilloma virus, head and neck cancer, renal cancer, and renal cell carcinoma, and wherein the patient is a human.

12. The method of claim 11, wherein the cancer is non-small-cell lung cancer, and wherein the patient has at least one of:
1. a predetermined tumor proportion score (TPS) of PD-L1 of <1%,
2. a tumor proportion score (TPS) of PD-L1 of 1%-49%, or
3. a predetermined absence of one or more driver mutations.

13. The method of claim 12, wherein the patient has a TPS of PD-L1 of <1%.

14. The method of any one of claims 10 to 13, wherein the patient has a cancer that is not indicated for treatment by an EGFR inhibitor, a BRAF inhibitor, an ALK inhibitor, a c-Ros inhibitor, a RET inhibitor, an ERBB2 inhibitor, BRCA inhibitor, a MAP2K1 inhibitor, PIK3CA inhibitor, CDKN2A inhibitor, a PTEN inhibitor, an UMD inhibitor, an NRAS inhibitor, a KRAS inhibitor, an NF1 inhibitor, MET inhibitor a TP53 inhibitor, a CREBBP inhibitor, a KMT2C inhibitor, a KMT2D mutation, an ARID1A mutation, a RB1 inhibitor, an ATM inhibitor, a SETD2 inhibitor, a FLT3 inhibitor, a PTPN11 inhibitor, a FGFR1 inhibitor, an EP300 inhibitor, a MYC inhibitor, an EZH2 inhibitor, a JAK2 inhibitor, a FBXW7 inhibitor, a CCND3 inhibitor, and a GNA11 inhibitor.

15. The method of any one of claims 10 to 14, wherein the patient has an absence of one or more driver mutations.

16. The method of claim 15, wherein the one or more driver mutations is selected from the group consisting of an EGFR mutation, an EGFR insertion, EGFR exon20, a KRAS mutation, a BRAF-mutation, a BRAF V600 mutation, an ALK-mutation, a c-ROS-mutation (ROS1-mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation.

17. The method of any one of claims 10 to 16, wherein the cancer is refractory or resistant to treatment with a chemotherapeutic agent or chemotherapeutic regimen.

18. The method of any one of claims 10 to 17, wherein the cancer is refractory or resistant to treatment with a VEGF-A inhibitor.

19. The method of claim 18, wherein the VEGF-A inhibitor is selected from the group consisting of bevacizumab, ranibizumab, icrucumab, and fragments, variants, and biosimilars thereof.

20. The method of any one of claims 10 to 19, wherein the cancer is refractory or resistant to treatment with a PD-1 inhibitor or PD-L1 inhibitor.

21. The method of claim 20, wherein the PD-1 or PD-L1 inhibitor is selected from the group consisting of nivolumab, pembrolizumab, cemiplimab, tislelizumab, sintilimab, toripalimab, dostarlimab, durvalumab, avelumab, atezolizumab, retifanlimab, and fragments, variants, and biosimilars thereof.

22. The method of any one of claims 10 to 21, wherein the cancer is refractory or resistant to treatment with a CTLA-4 inhibitor.

23. The method of claim 22, wherein the CTLA-4 inhibitor is selected from the group consisting of ipilimumab, tremelimumab, zalifrelimab, and fragments, variants, and biosimilars thereof.

24. The method of any one of claims 10 to 23, wherein the IL-2 is initially present at an initial concentration of between 1000 IU/mL and 6000 IU/mL in the first cell culture medium and in the second cell culture medium.

25. The method of any one of claims 10 to 24, wherein the OKT-3 antibody is initially present at an initial concentration of about 30 ng/mL in the second cell culture medium.

26. The method of any one of claims 10 to 25, wherein the first or second cell culture medium further comprises a cytokine selected from the group consisting of IL-4, IL-7, IL-15, IL-21, a 4-1BB agonist, an OX-40 agonist, an AKT inhibitor, and combinations thereof.

27. The method of any one of claims 10 to 26, wherein the second cell culture medium further comprises a cytokine selected from the group consisting of IL-4, IL-7, IL-15, IL-21, and combinations thereof.

28. The method of any one of claims 10 to 27, further comprising the step of treating the patient with a non-myeloablative lymphodepletion regimen prior to administering the third population of TILs to the patient.

29. The method of claim 28, wherein the non-myeloablative lymphodepletion regimen comprises the steps of administration of cyclophosphamide at a dose of 60 mg/m²/day for two days followed by administration of fludarabine at a dose of 25 mg/m²/day for five days.

30. The method of claim 28, wherein the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

31. The method of any one of claims 10 to 30, further comprising the step of treating the patient with an IL-2 regimen starting on the day after administration of the third population of TILs to the patient.

32. The method of any one of claims 10 to 31, further comprising the step of treating the patient with an IL-2 regimen starting on the same day as administration of the third population of TILs to the patient.

33. The method of any one of claims 31 to 32, wherein the IL-2 regimen is a high-dose IL-2 regimen comprising 600,000 or 720,000 IU/kg of aldesleukin, or a fragment, variant, or biosimilar thereof, administered as a 15-minute bolus intravenous infusion every eight hours until tolerance.

34. The methods of any one of claims 31 to 32, wherein the IL-2 regimen comprises administration of bempegaldesleukin, or a fragment, variant, or biosimilar thereof.

35. The methods of any one of claims 31 to 32, wherein the IL-2 regimen comprises administration of THOR-707, or a fragment, variant, or biosimilar thereof.

36. The methods of any one of claims 31 to 32, wherein the IL-2 regimen comprises administration of nemvaleukin alfa, or a fragment, variant, or biosimilar thereof.

37. The methods of any one of claims 31 to 32, wherein the IL-2 regimen comprises administration of an antibody comprising a heavy chain selected from the group consisting of SEQ ID NO:29 and SEQ ID NO:38 and a light chain selected from the group consisting of SEQ ID NO:37 and SEQ ID NO:39, or a fragment, variant, or biosimilar thereof.

38. The method of any one of claims 10 to 37, wherein a therapeutically effective population of TILs is administered and comprises from about 2×10⁹ to about 15×10¹⁰ TILs.

39. The method of any one of claims 10 to 38, wherein the first expansion is performed over a period of 11 days or less.

40. The method of any one of claims 10 to 39, wherein the second expansion is performed over a period of 11 days or less.

41. A composition comprising a tumor infiltrating lymphocyte (TIL), marrow infiltrating lymphocyte (MIL), or peripheral blood lymphocyte (PBL) genetically modified to express a chimeric costimulatory receptor (CCR), wherein the CCR comprises:
i. An extracellular domain,
ii. A hinge domain,
iii. A transmembrane domain, and
iv. At least one intracellular domain.

42. The composition of claim 41, wherein the extracellular domain comprises an scFv binding domain.

43. The composition of claim 42, wherein the scFv binding domain is selected from the group consisting of an anti-DLL3 domain, an anti-CD19 domain, an anti-CD20 domain, an anti-CD22 domain, an anti-CD24 domain, an anti-CD33 domain, an anti-CD38 domain, an anti-CD39 domain, an anti-CD73 domain, an anti-CD123 domain, an anti-CD138 domain, an anti-CD228 domain, an anti-LRRC15 domain, an anti-CEA domain, an anti-FRα domain, an anti-EPCAM domain, an anti-PD-L1 domain, an anti-PSMA domain, an anti-gp100 domain, an anti-MUC1 domain, an anti-MCSP domain, an anti-EGFR domain, an anti-GD2 domain, an anti-TROP-2 domain, an anti-GPC3 domain, an anti-MICA domain, an anti-MICB domain, an anti-VISTA domain, an anti-ULBP domain, an anti-HER2 domain, an anti-MCM5 domain, an anti-FAP domain, an anti-5T4 domain, an anti-LFA-1 domain, an anti-B7-H3 domain, and an anti-MUC16 domain.

44. The composition of claim 41, wherein the extracellular domain is a PD-1 domain, a FAS domain, or a TGFβRII domain.

45. The composition of any one of claims 41 to 44, wherein the intracellular domain is selected from the group consisting of a CD28 domain, a CD134 (OX40) domain, a CD278 (ICOS) domain, a CD137 (4-1BB) domain, a CD27 domain, a STAT3 domain, an IL-2Rβ domain, an IL-2Rγ domain, an IL-18R1 domain, an IL-18RAP domain, an IL-7Rα domain, an IL-12R1 domain, an IL-12R2 domain, an IL-15Rα domain, an IL-21R domain, and combinations thereof.

46. The composition of any one of claims 41 to 45, wherein the transmembrane domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, a IgG1 domain, a IgG4 domain, a IgD domain, a IL-2Rα domain, a IL-2Rβ domain, and a IL-2Rγ domain.

47. The composition of any one of claims 41 to 46, wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), and combinations thereof.

48. A composition comprising a chimeric costimulatory receptor (CCR), wherein the CCR comprises:
i. An extracellular protein domain,
ii. A hinge protein domain,
iii. A transmembrane protein domain, and
iv. At least one intracellular protein domain.

49. The composition of claim 48, wherein the extracellular protein domain comprises an scFv binding domain.

50. The composition of claim 49, wherein the scFv binding domain is selected from the group consisting of an anti-DLL3 domain, an anti-CD19 domain, an anti-CD20 domain, an anti-CD22 domain, an anti-CD24 domain, an anti-CD33 domain, an anti-CD38 domain, an anti-CD39 domain, an anti-CD73 domain, an anti-CD123 domain, an anti-CD138 domain, an anti-CD228 domain, an anti-LRRC15 domain, an anti-CEA domain, an anti-FRα domain, an anti-EPCAM domain, an anti-PD-L1 domain, an anti-PSMA domain, an anti-gp100 domain, an anti-MUC1 domain, an anti-MCSP domain, an anti-EGFR domain, an anti-GD2 domain, an anti-TROP-2 domain, an anti-GPC3 domain, an anti-MICA domain, an anti-MICB domain, an anti-VISTA domain, an anti-ULBP domain, an anti-HER2 domain, an anti-MCM5 domain, an anti-FAP domain, an anti-5T4 domain, an anti-LFA-1 domain, an anti-B7-H3 domain, an anti-IL-13Rα2 domain, an anti-FAS domain, an anti-TGFβRII domain, and an anti-MUC16 domain.

51. The composition of claim 48, wherein the extracellular protein domain is a PD-1 domain, a FAS domain, or a TGFβRII domain.

52. The composition of any one of claims 48 to 51, wherein the intracellular protein domain is selected from the group consisting of a CD28 domain, a CD134 (OX40) domain, a CD278 (ICOS) domain, a CD137 (4-1BB) domain, a CD27 domain, an IL-2Rβ domain, an IL-2Rγ domain, an IL-18R1 domain, an IL-18RAP domain, an IL-7Rα domain, an IL-12R1 domain, an IL-12R2 domain, an IL-15Rα domain, an IL-21R domain, and combinations thereof.

53. The composition of any one of claims 48 to 52, wherein the transmembrane protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain.

54. The composition of any one of claims 48 to 53, wherein the hinge protein domain is selected from the group consisting of a CD3α domain, a CD3β domain, a CDζ domain, a CD3ε domain, a CD4 domain, a CD5 domain, a CD8α domain, a CD9 domain, a CD16 domain, a CD22 domain, a CD27 domain, a CD28 domain, a CD33 domain, a CD37 domain, a CD45 domain, a CD64 domain, a CD80 domain, a CD86 domain, a CD134 domain, a CD137 domain, a CD154 domain, an IgG1 domain, an IgG4 domain, an IgD domain, an IL-2Rα domain, an IL-2Rβ domain, and an IL-2Rγ domain.

55. The composition of any one of claims 48 to 54, further comprising a tumor infiltrating lymphocyte.

56. The composition of any one of claims 48 to 54, further comprising a marrow infiltrating lymphocyte.

57. The composition of any one of claims 48 to 54, further comprising a peripheral blood lymphocyte.

58. A method of treating a cancer by administering a population of tumor infiltrating lymphocytes (TILs), marrow infiltrating lymphocytes (MILs), or peripheral blood lymphocytes (PBLs) to a patient in need thereof, wherein the TILs, MILs, or PBLs are genetically modified to express a chemokine receptor.

59. The method of claim 58, wherein the cancer is treated by administering a population of TILs, wherein the method comprises:
(a) obtaining and/or receiving a first population of TILs from a tumor resected from the patient by processing a tumor sample obtained from the patient into multiple tumor fragments or into a tumor digest;
(b) adding the first population of TILs into a closed system;
(c) performing a first expansion by culturing the first population of TILs in a first cell culture medium comprising IL-2 and optionally OKT-3 antibody and antigen presenting cells (APCs) to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) genetically modifying the second population of TILs to express the chemokine receptor;
(e) performing a second expansion of the second population of TILs in a second cell culture medium comprising IL-2, OKT-3 antibody, and APCs, to produce a third population of TILs, wherein the second expansion is performed for about 3-14 days to obtain the third population of TILs, wherein the third population of TILs is a therapeutic population of TILs, and wherein the second expansion is performed in a closed container providing a second gas-permeable surface area;
(f) harvesting a therapeutic population of TILs obtained from step (e);
(g) transferring the harvested TIL population from step (f) to an infusion bag, wherein the transfer from step (e) to (f) occurs without opening the system;
(h) cryopreserving the infusion bag comprising the harvested TIL population from step (f) using a cryopreservation process; and
(i) administering a therapeutically effective dosage of the third population of TILs from the infusion bag in step (g) to the patient.

60. The method of claim 59, wherein the chemokine receptor is a protein selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof.

61. The method of any one of claims 58 to 60, wherein step (d) further comprises genetically modifying TILs using a lentivirus or retrovirus to express the chemokine receptor.

62. The method of any one of claims 58 to 61, wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), SOCS1, ANKRD11, BCOR, and combinations thereof.

63. The method of any one of claims 58 to 62, wherein the cancer is a solid tumor cancer treated by administration of TILs.

64. The method of claim 63, wherein the cancer is selected from the group consisting of sarcoma, pancreatic cancer, liver cancer, glioblastoma, gastrointestinal cancer, melanoma, ovarian cancer, endometrial cancer, thyroid cancer, colorectal cancer, cervical cancer, lung cancer, non-small-cell lung cancer, small-cell lung cancer, mesothelioma, bladder cancer, breast cancer, cancer caused by human papilloma virus, head and neck cancer, renal cancer, and renal cell carcinoma, and wherein the patient is a human.

65. The method of claim 64, wherein the cancer is non-small-cell lung cancer, and wherein the patient has at least one of:
1. a predetermined tumor proportion score (TPS) of PD-L1 of <1%,
2. a tumor proportion score (TPS) of PD-L1 of 1%-49%, or
3. a predetermined absence of one or more driver mutations.

66. The method of claim 65, wherein the patient has a TPS of PD-L1 of <1%.

67. The method of any one of claims 63 to 66, wherein the patient has a cancer that is not indicated for treatment by an EGFR inhibitor, a BRAF inhibitor, an ALK inhibitor, a c-Ros inhibitor, a RET inhibitor, an ERBB2 inhibitor, BRCA inhibitor, a MAP2K1 inhibitor, PIK3CA inhibitor, CDKN2A inhibitor, a PTEN inhibitor, an UMD inhibitor, an NRAS inhibitor, a KRAS inhibitor, an NF1 inhibitor, MET inhibitor a TP53 inhibitor, a CREBBP inhibitor, a KMT2C inhibitor, a KMT2D mutation, an ARID1A mutation, a RB1 inhibitor, an ATM inhibitor, a SETD2 inhibitor, a FLT3 inhibitor, a PTPN11 inhibitor, a FGFR1 inhibitor, an EP300 inhibitor, a MYC inhibitor, an EZH2 inhibitor, a JAK2 inhibitor, a FBXW7 inhibitor, a CCND3 inhibitor, and a GNA11 inhibitor.

68. The method of any one of claims 63 to 66, wherein the patient has an absence of one or more driver mutations.

69. The method of claim 68, wherein the one or more driver mutations is selected from the group consisting of an EGFR mutation, an EGFR insertion, EGFR exon20, a KRAS mutation, a BRAF-mutation, a BRAF V600 mutation, an ALK-mutation, a c-ROS-mutation (ROS1-mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation.

70. The method of any one of claims 63 to 69, wherein the cancer is refractory or resistant to treatment with a chemotherapeutic agent or chemotherapeutic regimen.

71. The method of any one of claims 63 to 70, wherein the cancer is refractory or resistant to treatment with a VEGF-A inhibitor.

72. The method of claim 71, wherein the VEGF-A inhibitor is selected from the group consisting of bevacizumab, ranibizumab, icrucumab, and fragments, variants, and biosimilars thereof.

73. The method of any one of claims 63 to 72, wherein the cancer is refractory or resistant to treatment with a PD-1 inhibitor or PD-L1 inhibitor.

74. The method of claim 73, wherein the PD-1 or PD-L1 inhibitor is selected from the group consisting of nivolumab, pembrolizumab, cemiplimab, tislelizumab, sintilimab, toripalimab, dostarlimab, durvalumab, avelumab, atezolizumab, retifanlimab, and fragments, variants, and biosimilars thereof.

75. The method of any one of claims 63 to 74, wherein the cancer is refractory or resistant to treatment with a CTLA-4 inhibitor.

76. The method of claim 75, wherein the CTLA-4 inhibitor is selected from the group consisting of ipilimumab, tremelimumab, zalifrelimab, and fragments, variants, and biosimilars thereof.

77. The method of any one of claims 63 to 76, wherein the IL-2 is initially present at an initial concentration of between 1000 IU/mL and 6000 IU/mL in the first cell culture medium and in the second cell culture medium.

78. The method of any one of claims 63 to 77, wherein the OKT-3 antibody is initially present at an initial concentration of about 30 ng/mL in the second cell culture medium.

79. The method of any one of claims 63 to 78, wherein the first cell culture medium further comprises a cytokine selected from the group consisting of IL-4, IL-7, IL-15, IL-21, and combinations thereof.

80. The method of any one of claims 63 to 79, wherein the second cell culture medium further comprises a cytokine selected from the group consisting of IL-4, IL-7, IL-15, IL-21, and combinations thereof.

81. The method of any one of claims 63 to 80, further comprising the step of treating the patient with a non-myeloablative lymphodepletion regimen prior to administering the third population of TILs to the patient.

82. The method of claim 81, wherein the non-myeloablative lymphodepletion regimen comprises the steps of administration of cyclophosphamide at a dose of 60 mg/m²/day for two days followed by administration of fludarabine at a dose of 25 mg/m²/day for five days.

83. The method of claim 82, wherein the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

84. The method of any one of claims 63 to 83, further comprising the step of treating the patient with an IL-2 regimen starting on the day after administration of the third population of TILs to the patient.

85. The method of any one of claims 63 to 83, further comprising the step of treating the patient with an IL-2 regimen starting on the same day as administration of the third population of TILs to the patient.

86. The method of any one of claims 84 to 85, wherein the IL-2 regimen is a high-dose IL-2 regimen comprising 600,000 or 720,000 IU/kg of aldesleukin, or a fragment, variant, or biosimilar thereof, administered as a 15-minute bolus intravenous infusion every eight hours until tolerance.

87. The methods of any one of claims 84 to 85, wherein the IL-2 regimen comprises administration of bempegaldesleukin, or a fragment, variant, or biosimilar thereof.

88. The methods of any one of claims 84 to 85, wherein the IL-2 regimen comprises administration of THOR-707, or a fragment, variant, or biosimilar thereof.

89. The methods of any one of claims 84 to 85, wherein the IL-2 regimen comprises administration of nemvaleukin alfa, or a fragment, variant, or biosimilar thereof.

90. The methods of any one of claims 84 to 85, wherein the IL-2 regimen comprises administration of an antibody comprising a heavy chain selected from the group consisting of SEQ ID NO:29 and SEQ ID NO:38 and a light chain selected from the group consisting of SEQ ID NO:37 and SEQ ID NO:39, or a fragment, variant, or biosimilar thereof.

91. The method of any one of claims 63 to 90, wherein a therapeutically effective population of TILs is administered and comprises from about 2×10⁹ to about 15×10¹⁰ TILs.

92. The method of any one of claims 63 to 91, wherein the first expansion is performed over a period of 11 days or less.

93. The method of any one of claims 63 to 92, wherein the second expansion is performed over a period of 11 days or less.

94. A composition comprising a tumor infiltrating lymphocyte (TIL), marrow infiltrating lymphocyte (MIL), or peripheral blood lymphocyte (PBL) genetically modified to express a chemokine receptor.

95. The composition of claim 94, wherein the chemokine receptor is a protein selected from the group consisting of CXCR1, CXCR2, CXCR3, CXCR4, CXCR5, CXCR6, CXCR7 (ACKR3), CCR1, CCR2, CCR3, CCR4, CCR5, CCR6, CCR7, CCR8, CCR9, CCR10, CCR11, XCR1, CX3CR1, and combinations thereof.

96. The composition of any one of claims 94 to 95, wherein the TILs, MILs, or PBLs are further genetically modified to stably or transiently reduce the expression of a gene selected from the group consisting of PD-1, LAG-3, TIM-3, CTLA-4, TIGIT, CISH, TGFβR2, PKA, CBL-B, BAFF (BR3), and combinations thereof.

97. A composition comprising a chemokine receptor, wherein the composition further comprises a tumor infiltrating lymphocyte, a marrow infiltrating lymphocyte, or a peripheral blood lymphocyte.

98. A method or composition according to any of the preceding claims for use in combination with any of the preceding claims.
